(19) United States
(12) Patent Application Publication
(10) Pub. No.: US 2023/0220498 A1
(43) Pub. Date: Jul. 13, 2023

(54) Compositions and Methods for Detecting Respiratory Viruses Including Coronaviruses

(71) Applicant: THE TRUSTEES OF THE UNIVERSITY OF PENNSYLVANIA, Philadelphia, PA (US)

(72) Inventor: Erle S. Robertson, Wynnewood, PA (US)

(21) Appl. No.: 17/928,170

(22) PCT Filed: May 26, 2021
(86) PCT No.: PCT/US2021/034195

§ 371 Date: Nov. 28, 2022

Related U.S. Application Data
(60) Provisional application No. 63/032,048, filed on May 29, 2020.

(51) Int. Cl. C12Q 1/70

(52) U.S. Cl. CPC C12Q 1/701; C12Q 1/689

(57) ABSTRACT

The present invention includes compositions and methods for the detection of all known respiratory agents including Coronaviruses, and the recently discovered Sars-CoV-2, the agent of the CoVID Pandemic. The invention further includes detection of immune signatures of patients infected with a Coronavirus as well as other infectious agents.

### CROSS-REFERENCE TO RELATED APPLICATION

The present application is entitled to priority under 35 U.S.C. § 119(e) to U.S. Provisional Patent Application No. 63/032,048 filed May 29, 2020, which is hereby incorporated by reference in its entirety herein.

### BACKGROUND OF THE INVENTION

The ongoing Covid-19 pandemic caused by the novel SARS-CoV-2 virus is one of the deadliest picornaviruses to date and the most infectious of all human coronaviruses. This virus is estimated to be around 15-20 times more contagious than the influenza virus and is responsible for more deaths than SARS and MERS combined. The disease apparently has greater impact on individuals who are older with the mortality rate directly proportional with increase in age. This has been striking in individuals who also have other health complications and co-morbidities including diabetes, cardio-pulmonary diseases, or disease related to other major organ systems. It is also important to note that individuals with compromised immune systems are at greater risk for development of more rapid onset of Covid-19 disease. This provides a strong rationale for protection of patients who are HIV positive with a broad range of malignancies including AIDS defining and on-defining cancers. Patients who have cancers and are HIV positive should therefore be categorized as high risk for rapid onset of Covid-19 disease and increased mortality.

A need exists for compositions and methods for not only detecting SARS-Cov-2 in patients, but identifying those individuals who are at increased risk of mortality based on their associated coinfections. The present invention addressess this need.

### SUMMARY OF THE INVENTION

As described herein, the present invention relates to compositions and methods for the detection of all known respiratory agents including Coronaviruses, as well as immune signatures of patients infected with a Coronavirus.

In one aspect, the invention includes a composition comprising a PathoChip v5, wherein the PathoChip v5 comprises over 60,000 probes specific for a plurality of microbes, and a plurality of probes specific for Coronaviruses.

In certain embodiments, the Coronavirus probes comprise at least one nucleotide sequence selected from the group consisting of SEQ ID NOs: 1-37. In certain embodiments, the Coronavirus probes consist of the nucleotide sequences set forth in SEQ ID NOs: 1-37.

In another aspect, the invention includes a composition comprising a PathoChip comprising a plurality of probes specific for a plurality of respiratory pathogens, a plurality of probes specific for Coronaviruses, and a plurality of probes specific for immune markers of innate and adaptive responses to infections, inflammation, immune disorders, metabolism, and toxicological responses.

In certain embodiments, the plurality of probes specific for immune markers of innate and adaptive responses comprise at least one nucleotide sequence selected from the group consisting of SEQ ID NOs: 38-4,671. In certain embodiments, the plurality of probes specific for immune markers of innate and adaptive responses consist of the nucleotide sequences set forth in SEQ ID NOs: 38-4,671. In certain embodiments, the Coronavirus probes comprise at least one nucleotide sequence selected from the group consisting of SEQ ID NOs: 1-37. In certain embodiments, the Coronavirus probes consist of the nucleotide sequences set forth in SEQ ID NOs: 1-37.

In another aspect, the invention includes a composition comprising a plurality of nucleic acids, wherein the nucleic acids comprise at least one nucleotide sequence selected from the group consisting of SEQ ID NOs: 1-37.

In another aspect, the invention includes a composition comprising a plurality of nucleic acids, wherein the nucleic acids consist of the nucleotide sequences set forth in SEQ ID NOs: 1-37.

In another aspect, the invention includes a composition comprising a plurality of nucleic acids, wherein the nucleic acids comprise at least one nucleotide sequence selected from the group consisting of SEQ ID NOs: 38-4,671.

In another aspect, the invention includes a composition comprising a plurality of nucleic acids, wherein the nucleic acids consist of the nucleotide sequences set forth in SEQ ID NOs: 38-4,671.

In certain embodiments, the composition comprises a microarray. In certain embodiments, the microarray comprises a biochip, glass slide, bead, or paper.

In another aspect, the invention includes a composition comprising an Immunome ChIP, wherein the Immunome ChIP comprises a plurality of probes specific for immune markers of innate and adaptive responses to infections, inflammation, immune disorders, metabolism, and toxicological responses.

In certain embodiments, the plurality of probes comprise at least one nucleotide sequence selected from the group consisting of SEQ ID NOs: 38-4,671. In certain embodiments, the plurality of probes consist of the nucleotide sequences set forth in SEQ ID NOs: 38-4,671.

In another aspect, the invention includes a method of detecting a Coronavirus in a sample from a subject. The method comprises hybridizing a detectably-labeled nucleic acid from the sample to a PathoChip v5 array, wherein the PathoChip v5 comprises over 60,000 probes specific for a plurality of microbes, and a plurality of probes specific for Coronaviruses. When the nucleic acid from the sample hybridizes to at least three Coronavirus probes on the PathoChip v5, a Coronavirus is detected in the sample.

In another aspect, the invention includes a method of identifying a high-risk subject. The method comprises hybridizing a detectably-labeled nucleic acid from a sample from the subject to a PathoChip v5 array, wherein the PathoChip v5 comprises over 60,000 probes specific for a plurality of microbes, and a plurality of probes specific for Coronaviruses. When the nucleic acid from the sample hybridizes to at least three Coronavirus probes on the PathoChip v5 and at least three probes specific for a microbe, the subject is identified as high-risk.

In another aspect, the invention includes a method of detecting a Coronavirus in a sample from a subject. The method comprises hybridizing a detectably-labeled nucleic acid from the sample to a PathoChip array, wherein the PathoChip comprises a plurality of probes specific for a plurality of respiratory pathogens, a plurality of probes specific for Coronaviruses, and a plurality of probes specific for immune markers of innate and adaptive responses to infections, inflammation, immune disorders, metabolism, and toxicological responses. When the nucleic acid from the sample hybridizes to at least three Coronavirus probes on the PathoChip, a Coronavirus is detected in the sample.

In another aspect, the invention includes a method of identifying a high-risk subject. The method comprises hybridizing a detectably-labeled nucleic acid from a sample from the subject to a PathoChip array, wherein the PathoChip comprises a plurality of probes specific for a plurality of respiratory pathogens, a plurality of probes specific for Coronaviruses, and a plurality of probes specific for immune markers of innate and adaptive responses to infections, inflammation, immune disorders, metabolism, and toxicological responses. When the nucleic acid from the sample hybridizes to at least three Coronavirus probes on the PathoChip and at least three probes specific for a immune markers of innate and adaptive responses, the subject is identified as high-risk.

In certain embodiments, the Coronavirus probes comprise at least one nucleotide sequence selected from the group consisting of SEQ ID NOs: 1-37. In certain embodiments, the Coronavirus probes consist of the nucleotide sequences set forth in SEQ ID NOs: 1-37.

In certain embodiments, when a Coronavirus is detected in the sample from the subject or the subject is identified as high-risk, then the subject is administered a treatment for the Coronavirus.

In certain embodiments, the plurality of probes specific for immune markers of innate and adaptive responses comprises at least one nucleotide sequence selected from the group consisting of SEQ ID NOs: 38-4,671. In certain embodiments, the plurality of probes specific for immune markers of innate and adaptive responses consists of the nucleotide sequences set forth in SEQ ID NOs: 38-4,671.

In certain embodiments, the subject is human.

In certain embodiments, the detectably-labeled nucleic acid is labeled with a fluorophore, radioactive phosphate, biotin, or enzyme. In certain embodiments, the fluorophore is Cy3 or Cy5.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 illustrates the PathoChip and it's various versions. Version 5 (v5) is disclosed herein and comprises over 60,000 probes specific for viruses, helminths, protozoa, fungi, and bacteria (Banerjee et al. (2015) Sci Rep. 5:15162,10.1038/srep15162; Baldwin et al. (2014) MBio. 5, e01714-01714. doi: 01710.01128/mBio.01714-01714), in addition to 37 probes specific for Coronaviruses (SEQ ID NOs: 1-37).

FIG. 2 illustrates selection of Coronavirus (including SARS-CoV-2) probes.

FIGS. 3A-3B illustrate results of validation of the Coronavirus probes with RNA from a patient sample showing the strong hybridization of SARS-CoV-2 probes with viral genomic RNA to unique, conserved probes with mutations that were commonly seen in some SARS-CoV-2 genomic sequences. It also shows specificity of the probes to regions that were included as genomic sections that should not hybridize to the probes and some that have hybridization to probes demonstrating specificity. Panel B shows a box plot of signals for SARS-CoV-2 probe sets (unique, conserved and mutant probes).

FIG. 4 illustrates probe ID and signal intensity. The top left panel shows the signal obtained from human genomic nucleic acid with minimal detection of some probes. Specifically with different amounts of SARS-COV-2 RNA the entire probe set are detected with some having great intensities.

FIG. 5 illustrates nucleotide sequences of the Coronavirus probes selected.

FIG. 6 illustrates total immunome signatures that can be detected by the Immunome ChIP which includes areas of infections, immune disorders, metabolism, toxicology, and inflammatory response, which covers all know immune markers.

FIGS. 7A-7E illustrate cytokine chip symbols which are known to be associated with the different immune responses. The blue bars on the left show the responses that are unique or conserved across the 5 different types of immune responses and the total for each grouping shown.

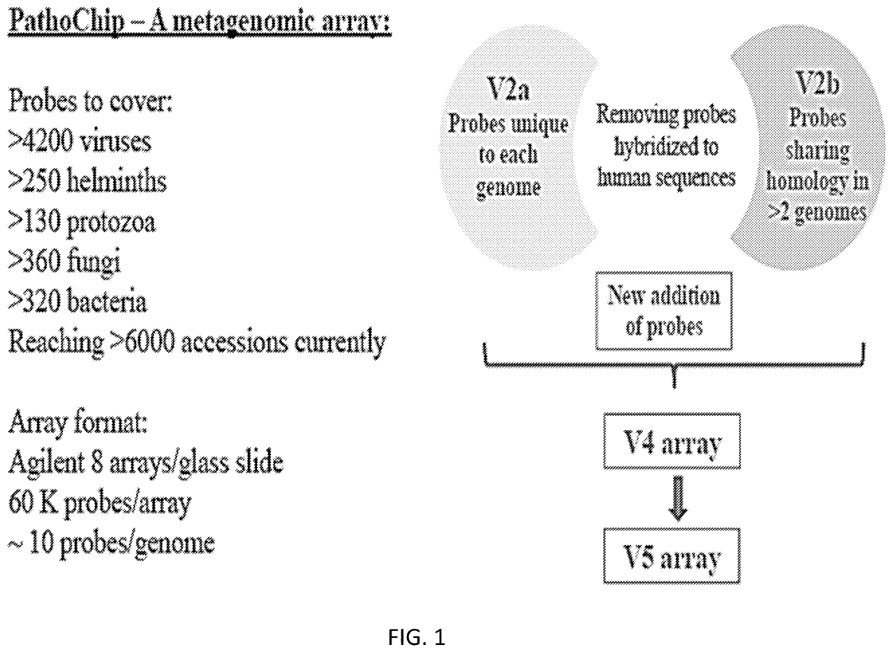

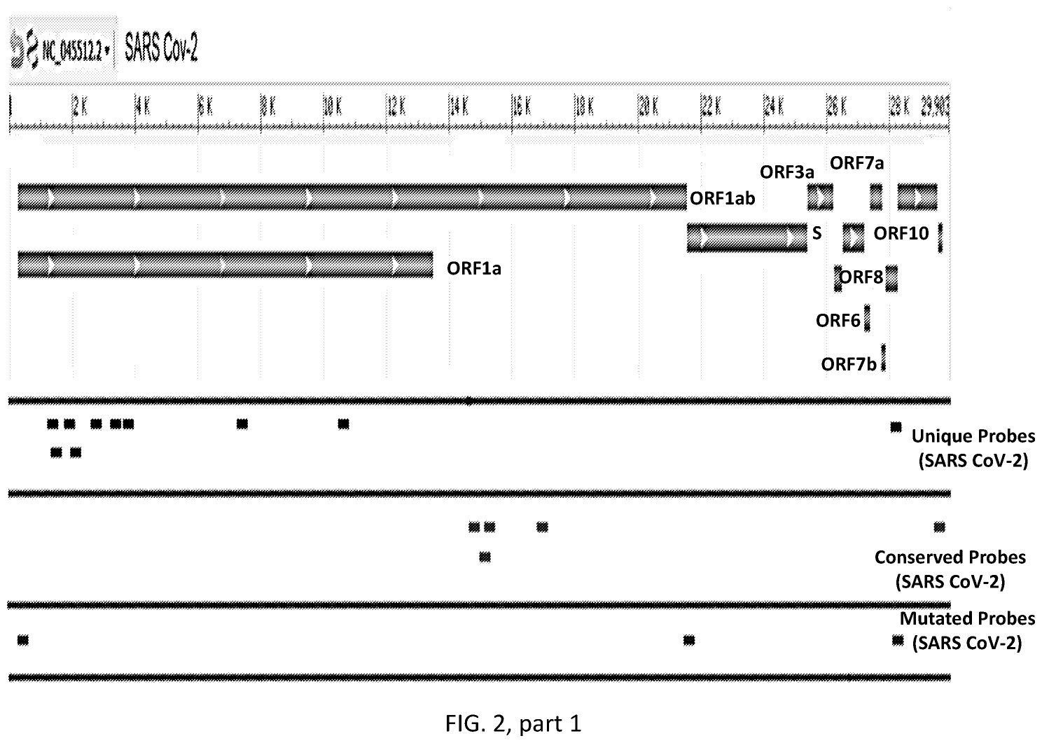

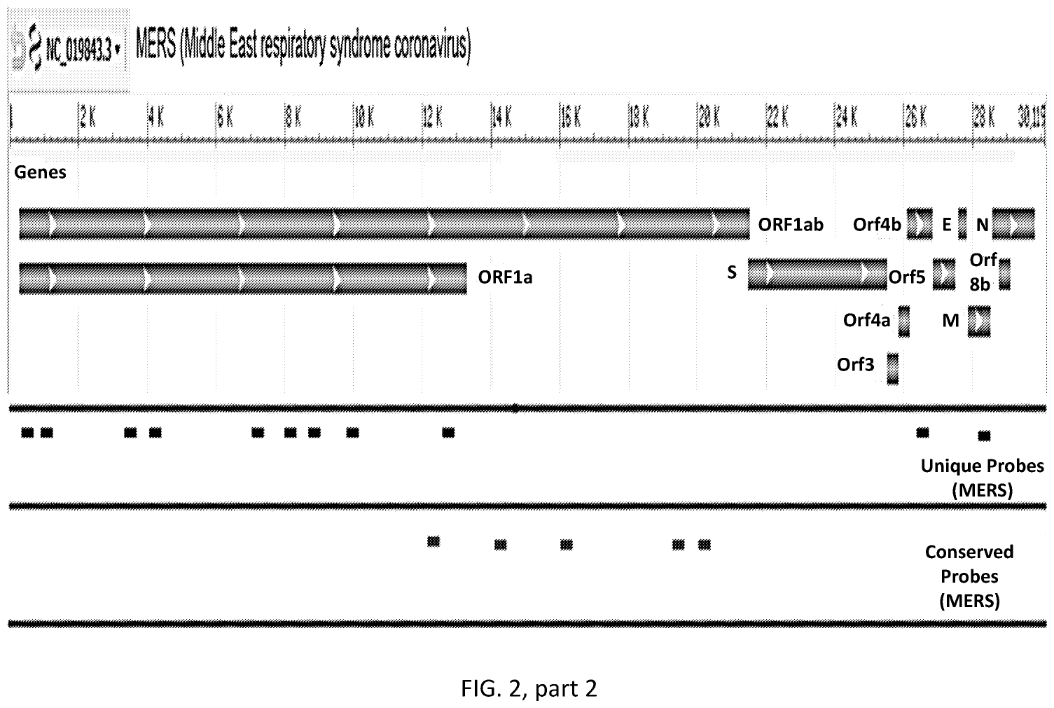

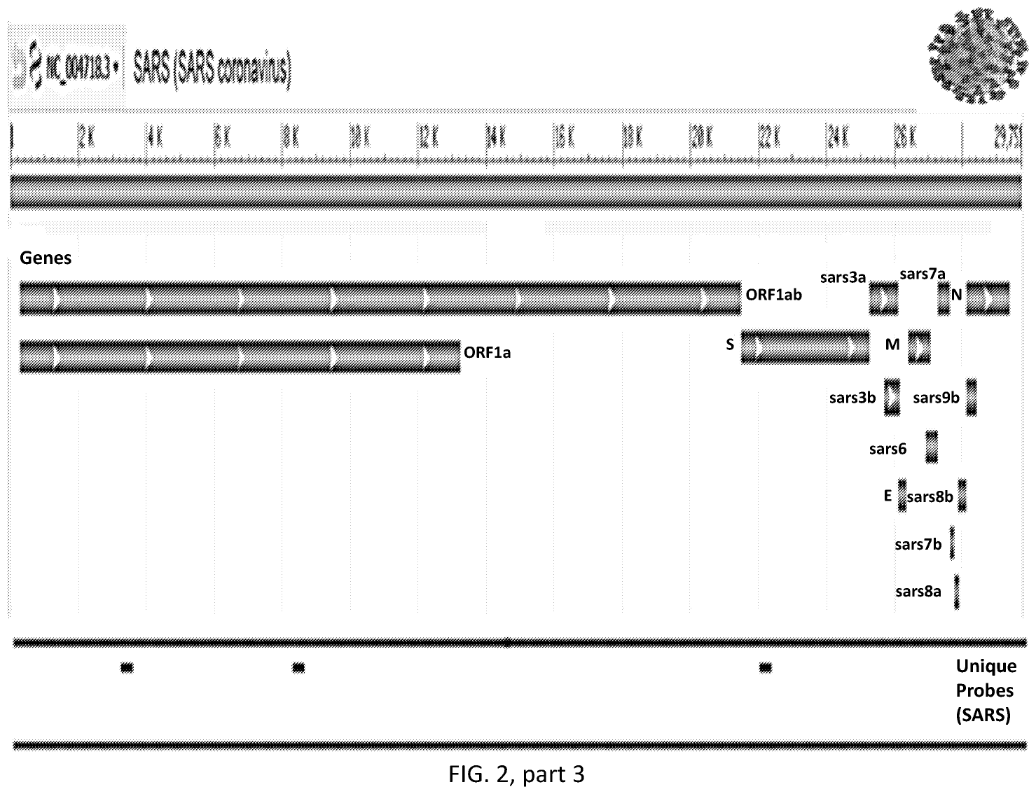

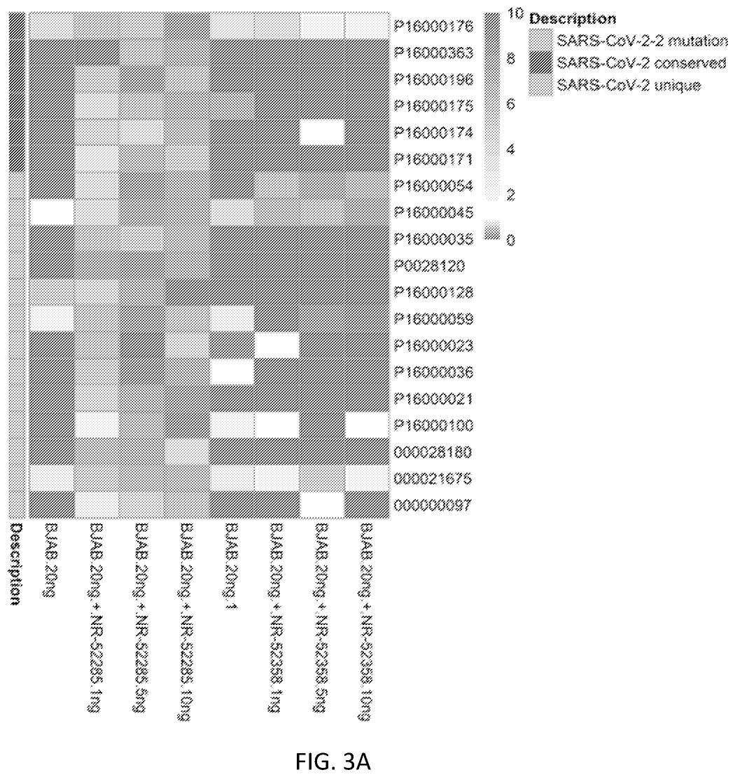

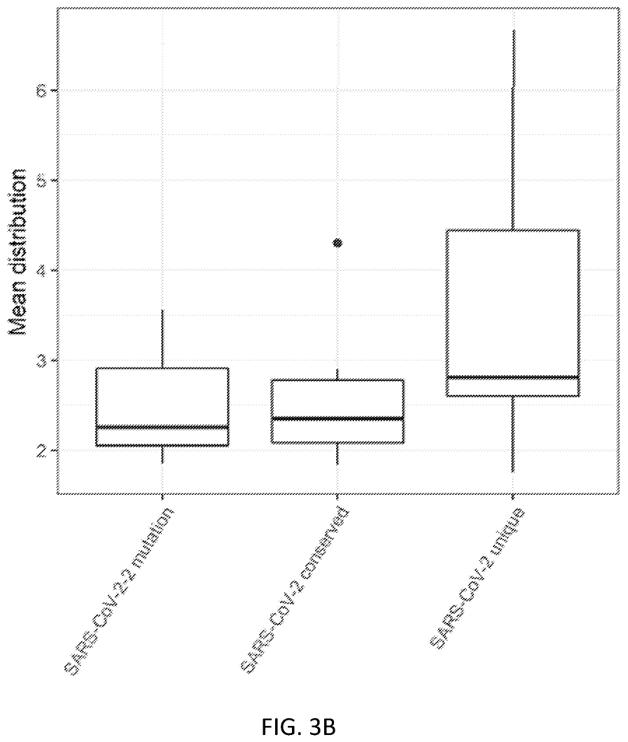

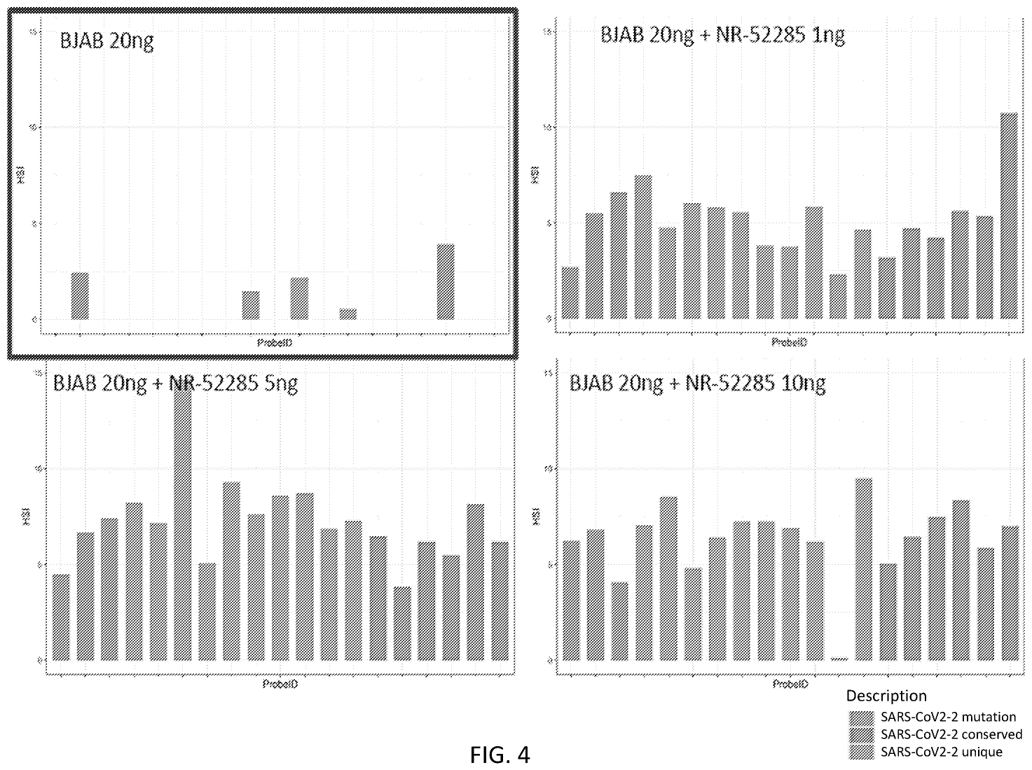

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a”, “an”, and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

A “biomarker” or “marker” as used herein generally refers to a nucleic acid molecule, clinical indicator, protein, or other analyte that is associated with a disease. In certain embodiments, a nucleic acid biomarker is indicative of the presence in a sample of a pathogenic organism, including but not limited to, viruses, viroids, bacteria, fungi, helminths, and protozoa. In various embodiments, a marker is differentially present in a biological sample obtained from a subject having or at risk of developing a disease (e.g., an infectious disease) relative to a reference. A marker is differentially present if the mean or median level of the biomarker present in the sample is statistically different from the level present in a reference. A reference level may be, for example, the level present in an environmental sample obtained from a clean or uncontaminated source. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease (e.g., an infectious disease), for determining the prognosis of the subject, for evaluating therapeutic efficacy, or for selecting a treatment regimen.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

“Coronaviruses” as used herein is meant a positive, single-stranded enveloped RNA viruse belonging to the Coronaviridae family, including but not limited to, SARS-CoV-2, SARS-CoV, MERS-CoV, 229E (alpha coronavirus), NL63 (alpha coronavirus), OC43 (beta coronavirus), HKU1 (beta coronavirus), 2019-nCoV, and and other common Coronaviruses associated with the common cold.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Hybridization” means hydrogen bonding, which may be Watson-Crick, Hoogsteen or reversed Hoogsteen hydrogen bonding, between complementary nucleobases. For example, adenine and thymine are complementary nucleotides that pair through the formation of hydrogen bonds.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker profile” is meant a characterization of the signal, level, expression or expression level of two or more markers (e.g., polynucleotides).

By the term “microbe” is meant any and all organisms classed within the commonly used term “microbiology,” including but not limited to, bacteria, viruses, fungi and parasites.

By the term “microarray” is meant a collection of nucleic acid probes immobilized on a substrate. As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring. Nucleic acid molecules useful in the methods of the invention include any nucleic acid molecule that specifically binds a target nucleic acid (e.g., a nucleic acid biomarker). Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By “reference” is meant a standard of comparison. As is apparent to one skilled in the art, an appropriate reference is where an element is changed in order to determine the effect of the element. In one embodiment, the level of a target nucleic acid molecule present in a sample may be compared to the level of the target nucleic acid molecule present in a clean or uncontaminated sample. For example, the level of a target nucleic acid molecule present in a sample may be compared to the level of the target nucleic acid molecule present in a corresponding healthy cell or tissue or in a diseased cell or tissue (e.g., a cell or tissue derived from a subject having a disease, disorder, or condition).

As used herein, the term “sample” includes a biologic sample such as any tissue, cell, fluid, or other material derived from an organism.

By “specifically binds” is meant a compound (e.g., nucleic acid probe or primer) that recognizes and binds a molecule (e.g., a nucleic acid biomarker), but which does not substantially recognize and bind other molecules in a sample, for example, a biological sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

By “target nucleic acid molecule” is meant a polynucleotide to be analyzed. Such polynucleotide may be a sense or antisense strand of the target sequence. The term “target nucleic acid molecule” also refers to amplicons of the original target sequence. In various embodiments, the target nucleic acid molecule is one or more nucleic acid biomarkers.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By the term “tumor tissue sample” is meant any sample from a tumor in a subject including any solid and non-solid tumor in the subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention features compositions and methods for detecting a Coronavirus, including SARS-CoV-2, in a subject. Detection of the Coronavirus is made in conjunction with detection of other co-indications or co-morbidities (e.g. bacterial, viral, or parasitic infections or cancer). A subject's immune response to the Cornavirus can also be determined using the methods disclosed herein.

In one embodiment, a diagnostic test was developed herein for accurately identifying the SARS-CoV-2 coronavirus recently identified as the causative agent of the Covid-19 disease. The system uses the microarray platform of 8 arrays of 60,000 probes for detection of all known viruses and other pathogenic bacteria, fungi and parasites. This covers over 6,000 accessions of microorganisms associated with diseases including all known respiratory pathogens. The technology was modified to include new virus SARS-CoV-2 probes and can detect these organisms in as rapid as 24 hours. At the same time probes were engineered bioinformatically for over 1720 immune markers to cover all innate and adaptive responses to infections, inflammatory, immune disorders, metabolism and toxicological responses. Unique probes were generated that would provide a snapshot of the immune response to SARS-CoV-2 which will allow for stratification of the patients who may be at greater risk for development of disease severity. The test at the same time covers all known respiratory pathogens including other coronaviruses from bats, and other mammals providing accurate and efficient detection of SARS-CoV-2 as well as potential for reflex of other respiratory pathogens and cytokines associated with the Covid-19 disease. Therefore the benefit of the test is in the sensitivity, accuracy by using multiple probes across the genome to avoid negative results if one probe fails in the assay screen. The overall screen can be used for research as well as clinical sample testing for detection of SARS-Cov2 coronavirus with high sensitivity and accuracy.

### Compositions

In one aspect, the invention includes a composition comprising a PathoChip v5, wherein the PathoChip v5 comprises over 60,000 probes specific for a plurality of microbes, and a plurality of probes specific for Coronaviruses. In certain embodiments, the Coronavirus probes comprise at least one nucleotide sequence selected from the group consisting of SEQ ID NOs: 1-37. In certain embodiments, the Coronavirus probes consist of the nucleotide sequences set forth in SEQ ID NOs: 1-37.

In another aspect, the invention includes a PathoChip that further comprises a plurality of additional probes specific for immune markers of innate and adaptive responses to infections, inflammation, immune disorders, metabolism, and toxicological responses. This version of the PathoChip is also referred to as the ImmunoChIP. In certain embodiments, the PathoChip (ImmunoChIP) comprises 15,000 probes which specific for all know respiratory pathogens including Coronaviruses, sexually transmitted agents, food borne pathogens and other common transmissible agents. In certain embodiments, the Coronavirus probes comprise at least one nucleotide sequence selected from the group consisting of SEQ ID NOs: 1-37. In certain embodiments, the Coronavirus probes consist of the nucleotide sequences set forth in SEQ ID NOs: 1-37. In certain embodiments, the plurality of additional probes are specific for 1,728 known immune markers, that are in response to biological or physical assaults to humans. In certain embodiments, the plurality of additional probes comprise at least one nucleotide sequence selected from the group consisting of SEQ ID NOs: 38-4,671. In certain embodiments, the plurality of additional probes consist of the nucleotide sequences set forth in SEQ ID NOs: 38-4,671.

In another aspect, the invention includes a composition comprising a plurality of nucleic acids, wherein the nucleic acids comprise at least one nucleotide sequence selected from the group consisting of SEQ ID NOs: 1-37.

In another aspect, the invention includes a composition comprising a plurality of nucleic acids, wherein the nucleic acids consist of the nucleotide sequences set forth in SEQ ID NOs: 1-37.

In another aspect, the invention includes a composition comprising a plurality of nucleic acids, wherein the nucleic acids comprise at least one nucleotide sequence selected from the group consisting of SEQ ID NOs: 38-4,671.

In another aspect, the invention includes a composition comprising a plurality of nucleic acids, wherein the nucleic acids consist of the nucleotide sequences set forth in SEQ ID NOs: 38-4,671.

In certain embodiments, the composition comprises a microarray. In certain embodiments, the microarray comprises a biochip, glass slide, bead, or paper.

In another aspect, the invention includes a composition comprising an Immunome ChIP, wherein the Immunome ChIP comprises a plurality of probes specific for immune markers of innate and adaptive responses to infections, inflammation, immune disorders, metabolism, and toxicological responses. In certain embodiments the plurality of probes comprise at least one nucleotide sequence selected from the group consisting of SEQ ID NOs: 38-4,671. In certain embodiments the plurality of probes consist of the nucleotide sequences set forth in SEQ ID NOs: 38-4,671.

Also provided are kits comprising the compositions disclosed herein or for practicing the invention methods, as described herein. For example, in some embodiments, the invention includes a kit comprising the PathoChip v5 or Immunome ChIP. Additional reagents that are required or desired in the protocol to be practiced with the kit components may be included with the kit. Additional reagents can include, but are not limited to, supplementary nucleic acids, carriers, and PCR amplification reagents (e.g., nucleotides, buffers, cations, etc.), and the like. The kit components may be present in separate containers, or one or more of the components may be present in the same container, where the containers may be storage containers and/or containers that are employed during the assay for which the kit is designed.

In addition to the above components, the kit may further include instructions for practicing the methods described herein. The instructions will generally include information about the use of the composition for the detection of Coronaviruses. In certain embodiments, the instructions include information about how to use the kit. In certain embodiments the instructions include information on how to analyze and interpret the data generated from use of the kit. In other embodiments, the instructions include at least one of the following: description of the therapeutic agent; dosage schedule and administration for treatment or prevention of Coronavirus or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Methods

The present invention also includes methods of detecting Coronaviruses, methods of determining a response (e.g. an immune response) to an infection (e.g. COVID-19 infection), and methods of determining whether a patient is high-risk.

In one aspect, the invention includes a method of detecting a Coronavirus in a sample from a subject. The method comprises hybridizing a detectably-labeled nucleic acid from the sample to a PathoChip v5 array, wherein the PathoChip v5 comprises over 60,000 probes specific for a plurality of microbes, and a plurality of probes specific for Coronaviruses. When the nucleic acid from the sample hybridizes to at least three Coronavirus probes on the PathoChip v5, a Coronavirus is detected in the sample.

Coronaviruses that can be detected with the compositions and methods disclosed herein include, but are not limited to, SARS-CoV-2, SARS-CoV, MERS-CoV, 229E (alpha coronavirus), NL63 (alpha coronavirus), OC43 (beta coronavirus), HKU1 (beta coronavirus), and 2019-nCoV.

In another aspect, the invention includes a method of detecting a Coronavirus in a sample from a subject. The method comprises hybridizing a detectably-labeled nucleic acid from the sample to a PathoChip array, wherein when the nucleic acid from the sample hybridizes to at least three Coronavirus probes on the PathoChip, a Coronavirus is detected in the sample. In certain embodiments, the PathoChip comprises a plurality of probes specific for all respiratory pathogens, including Coronaviruses, and immune markers of innate and adaptive responses to infections, inflammation, immune disorders, metabolism, and toxicological responses. In such embodiments, use of the PathoChip results in simultaneous measurement of the Coronavirus, and the subject's response (e.g. immune response) to the virus. This version of the PathoChip is also referred to as the ImmunoChIP.

In another aspect, the invention includes a method of identifying a high-risk subject comprising hybridizing a detectably-labeled nucleic acid from a sample from the subject to a PathoChip v5 array, wherein when the nucleic acid from the sample hybridizes to at least three Coronavirus probes on the PathoChip v5 and at least three probes specific for a microbe, the subject is identified as high-risk.

In another aspect, the invention includes a method of identifying a high-risk subject comprising hybridizing a detectably-labeled nucleic acid from a sample from the subject to a PathoChip array, wherein the PathoChip comprises a plurality of probes specific for a plurality of respiratory pathogens, a plurality of probes specific for Coronaviruses, and a plurality of probes specific for immune markers of innate and adaptive responses to infections, inflammation, immune disorders, metabolism, and toxicological responses. When the nucleic acid from the sample hybridizes to at least three Coronavirus probes on the PathoChip and at least three probes specific for a immune markers of innate and adaptive responses, the subject is identified as high-risk.

In certain embodiments, the Coronavirus probes comprise at least one nucleotide sequence selected from the group consisting of SEQ ID NOs: 1-37. In certain embodiments, the Coronavirus probes consist of the nucleotide sequences set forth in SEQ ID NOs: 1-37.

In certain embodiments, the plurality of probes specific for immune markers of innate and adaptive responses comprise at least one nucleotide sequence selected from the group consisting of SEQ ID NOs: 38-4,671. In certain embodiments, the plurality of probes specific for immune markers of innate and adaptive responses consist of the nucleotide sequences set forth in SEQ ID NOs: 38-4,671.

In certain embodiments, when a Coronavirus is detected in the sample from the subject, the subject is administered a treatment for the Coronavirus.

The detectably-labeled nucleic acid can be labeled with a fluorophore (e.g Cy3 or Cy5), radioactive phosphate, biotin, or enzyme.

In certain embodiments, the subject is a human.

### Sample Preparation

The invention provides a means for analyzing multiple types of nucleic acids present in a sample, including DNA and RNA. In various embodiments, sample preparation involves extracting a mixture of nucleic acid molecules (e.g., DNA and RNA). In other embodiments, sample preparation involves extracting a mixture of nucleic acids from multiple organisms, cell types, infectious agents, or any combination thereof. In one embodiment, sample preparation involves the workflow below.

A. Fragment genomic DNA
B. Convert total RNA to first strand cDNA by random-primed reverse transcriptase
C. Label genomic DNA with biotin or fluorescent dye by chemical or enzymatic incorporation
D. Label cDNA with biotin or fluorescent dye by chemical or enzymatic incorporation
E. Label a mixture of genomic DNA and cDNA in the same chemical or enzymatic reaction
F. Mix C+D and co-hybridize to microarray of probes
G. Hybridize E to microarray of probes
H. Amplify targeted genomic DNA
1. Use whole-genome amplification (GE GenomiPhi, Sigma WGA, NuGEN Ovation DNA) to non-specifically amplify genomic DNA
2. Use amplified products as input for steps C or E above.
   I. Amplify targeted total RNA
1. Use whole-transcriptome amplification (Sigma WTA, Ambion in vitro transcription, NuGEN Ovation RNA) to non-specifically amplify total RNA
2. Use amplified products as input.

The samples are hybridized to the microarray (e.g., PathoChip and ImmunoChIP), and the microarrays are washed at various stringencies. Microarrays are scanned for detection of fluorescence. Background correction and inter-array normalization algorithms are applied. Detection thresholds are applied. The results are analyzed for statistical significance.

### Nucleic Acid Amplification

Target nucleic acid sequences are optionally amplified before being detected. The term “amplified” defines the process of making multiple copies of the nucleic acid from a single or lower copy number of nucleic acid sequence molecule. The amplification of nucleic acid sequences is carried out in vitro by biochemical processes known to those of skill in the art. Prior to or concurrent with identification, the viral sample may be amplified by a variety of mechanisms, some of which may employ PCR. For example, primers for PCR may be designed to amplify regions of the sequence. For RNA viruses a first reverse transcriptase step may be used to generate double stranded DNA from the single stranded RNA. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675. The sample may be amplified on the array. See, for example, U.S. Pat. No. 6,300,070 and U.S. Ser. No. 09/513,300.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed PCR (CP-PCR) (U.S. Pat. No. 4,437,975), arbitrarily primed PCR (AP-PCR) (U.S. Pat. Nos. 5,413,909, 5,861,245) and nucleic acid based sequence amplification (NABSA) (see, U.S. Pat. Nos. 5,409,818, 5,554,517, and 6,063,603). Other amplification methods that may be used are described in, U.S. Pat. Nos. 5,242,794, 5,494,810, 4,988,617 and in U.S. Ser. No. 09/854,317.

Additional methods of sample preparation and techniques for reducing the complexity of a nucleic acid sample are described in Dong et al., Genome Research 11, 1418 (2001), in U.S. Pat. Nos. 6,361,947, 6,391,592 and U.S. Ser. Nos. 09/916,135, 09/920,491 (US Patent Application Publication 20030096235), Ser. No. 09/910,292 (US Patent Application Publication 20030082543), and Ser. No. 10/013,598.

### Detection of Biomarkers

The biomarkers of this invention can be detected by any suitable method. The methods described herein can be used individually or in combination for a more accurate detection of the biomarkers. Methods for conducting polynucleotide hybridization assays have been developed in the art. Hybridization assay procedures and conditions will vary depending on the application and are selected in accordance with the general binding methods known including those referred to in: Sambrook and Russell, Molecular Cloning: A Laboratory Manual (3^{rd} Ed. Cold Spring Harbor, N.Y, 2001); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623. A data analysis algorithm (E-predict) for interpreting the hybridization results from an array is publicly available (see Urisman, 2005, Genome Biol 6:R78).

In one embodiment, the hybridized nucleic acids are detected by visualization of one or more labels attached to, or incorporated within, the sample nucleic acids. The labels may be attached or incorporated by any of a number of means well known to those of skill in the art. In one embodiment, the label is simultaneously incorporated during the amplification step in the preparation of the sample nucleic acids. Thus, for example, PCR with labeled primers or labeled nucleotides will provide a labeled amplification product. In another embodiment, transcription amplification, as described above, using a labeled nucleotide (e.g. fluorescein-labeled UTP and/or CTP) incorporates a label into the transcribed nucleic acids. In another embodiment PCR amplification products are fragmented and labeled by terminal deoxytransferase and labeled dNTPs. Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase.

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies, magnetic beads (e.g., Dynabeads™); fluorescent dyes (e.g., Cy3, Cy5, fluorescein, texas red, rhodamine, green fluorescent protein, and the like); radiolabels (e.g., ³H, ¹²⁵I, ³⁵S, ⁴C, or ³²P); phosphorescent labels; enzymes (e.g., horse radish peroxidase, alkaline phosphatase and others commonly used in an ELISA); and colorimetric labels such as colloidal gold or colored glass or plastic (e.g., polystyrene, polypropylene, latex, etc.) beads. Patents teaching the use of such labels include U.S. Pat. Nos. 3,817,837, 3,850,752, 3,939,350, 3,996,345, 4,277,437, 4,275,149 and 4,366,241.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964).

### Detection by Microarray

In certain aspects of the invention, a sample is analyzed by means of a microarray. The nucleic acid molecules of the invention are useful as hybridizable array elements in a microarray. Microarrays generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. U.S. Pat. Nos. 5,800,992 and 6,040,138 describe methods for making arrays of nucleic acid probes that can be used to detect the presence of a nucleic acid containing a specific nucleotide sequence. Methods of forming high-density arrays of nucleic acids, peptides and other polymer sequences with a minimal number of synthetic steps are known. The nucleic acid array can be synthesized on a solid substrate by a variety of methods, including, but not limited to, light-directed chemical coupling, and mechanically directed coupling. For additional descriptions and methods relating to resequencing arrays see U.S. patent application Ser. Nos. 10/658,879, 60/417,190, 09/381,480, 60/409,396, and U.S. Pat. Nos. 5,861,242, 6,027,880, 5,837,832, 6,723,503.

By “hybridize” is meant pairing to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The microarray can be a biochip, or on a glass slide, bead, or paper.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure. Exemplary nucleic acid molecules useful in the invention include polynucleotides that specifically bind nucleic acid biomarkers to one or more pathogenic organisms, and fragments thereof.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell or population of cells isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection systems for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In certain embodiments, a scanner is used to determine the levels and patterns of fluorescence.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual,” fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed herein.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the exemplary embodiments of the present invention, and are not to be construed as limiting in any way the remainder of the disclosure.

The materials and methods employed in these experiments are now described.

PathoChip design: The details of PathoChip Arrays v1, v2, v3, and v4 have been previously described (Banerjee et al. (2015) Sci Rep. 5:15162., 10.1038/srep15162; Baldwin et al. (2014) MBio. 5, e01714-01714. doi: 01710.01128/mBio.01714-01714). The PathoChip comprises 60,000 probe sets of sequenced microorganisms from Genbank, which are manufactured as SurePrint glass slide microarrays (Agilent Technologies Inc.), containing 8 replicate arrays per slide. Each probe is a 60-nt DNA oligomer that targets multiple genomic regions of pathogenic viruses, prokaryotic, and eukaryotic microorganisms. Specifically, there are an average of 5-10 target-specific probes per accession, and accessions include approx. 4,200 viruses, 320 bacteria, 360 fungi, 130 protozoa, and 250 helminths. Probes to target regions are conserved between virus families and saturation probe sets are included for selected viral agents.

The PathoChip technology, combined with PCR and NGS, is also a valuable strategy for detecting and identifying pathogens in breast, oropharyngeal, and ovarian cancers (Banerjee et al. (2015) Sci Rep. 5:15162., 10.1038/srep15162; Banerjee et al. (2017) Sci Rep. 7, 4036. doi: 4010.1038/s41598-41017-03466-41596; Banerjee et al. (2017) Oncotarget 8, 36225-36245. doi: 36210.18632/oncotarget.16717; Baldwin et al. (2014) MBio. 5, e01714-01714. doi: 01710.01128/mBio.01714-01714). Probes and accession annotations are available in the Gene Expression Omnibus (http://wwwdotncbinlmdotnihdotgov/geo/).

Sample preparation and Microarray processing: DNA and RNA was extracted from samples (Banerjee et al. (2015) Sci Rep. 5:15162., 10.1038/srep15162; Banerjee et al. (2017) Sci Rep. 7, 4036. doi: 4010.1038/s41598-41017-03466-41596; Banerjee et al. (2017) Oncotarget 8, 36225-36245. doi: 36210.18632/oncotarget.16717; Baldwin et al. (2014) MBio. 5, e01714-01714. doi: 01710.01128/mBio.01714-01714) and used for screening. The quality of extracted nucleic acids was determined by agarose gel electrophoresis and the A260/280 ratio. The extracted RNA and DNA samples were subjected to whole genome and transcriptome amplification (referred here as WTA) using TransPlex Complete Whole Transcriptome Amplification Kit (Sigma-Aldrich, St. Louis, Mo.) using 50 ng each of RNA and DNA as input and manufacturers protocol. The WTA products were analyzed by agarose gel electrophoresis and showed a range of 200-400 bp amplicon sizes and no contamination in the non-template control used during WTA. Human reference RNA and DNA were also extracted and 15 ng of each were used for WTA. The WTA products were purified, (PCR purification kit, Qiagen, Germantown, Md., USA), and 2 μg of the amplified products was labelled with Cy3 and that from the human reference was labelled with Cy5 (SureTag labeling kit, Agilent Technologies, Santa Clara, Calif.) as per manufacturer's protocol. Human reference DNA and RNA was used to determine cross-hybridization of probes to human DNA. The labelled cDNA/DNAs were purified and the efficiencies of labeling were determined by measuring absorbance at 550 nm (for Cy3) and 650 nm (for Cy5). The labelled samples (Cy3 plus Cy5) were hybridized to the PathoChip as described previously (Banerjee et al. (2015) Sci Rep. 5:15162., 10.1038/srep15162; Banerjee et al. (2017) Sci Rep. 7, 4036. doi: 4010.1038/s41598-41017-03466-41596; Banerjee et al. (2017) Oncotarget 8, 36225-36245. doi: 36210.18632/oncotarget.16717; Baldwin et al. (2014) MBio. 5, e01714-01714. doi: 01710.01128/mBio.01714-01714). The hybridization cocktail (CGH blocking agent and hybridization buffer), was added to each of the labeled test sample (Cy3) mixed with reference (Cy5), denatured and hybridized to the arrays in 8-chamber gasket slides. The slides were incubated at 65° C. with rotation and washed, then scanned for visualization using an Agilent SureScan G4900DA array scanner.

Microarray Data Extraction and Statistical analysis: Microarray data were extracted and analyzed as described previously (Banerjee et al. (2015) Sci Rep. 5:15162., 10.1038/srep15162; Banerjee et al. (2017) Sci Rep. 7, 4036. doi: 4010.1038/s41598-41017-03466-41596; Banerjee et al. (2017) Oncotarget 8, 36225-36245. doi: 36210.18632/oncotarget.16717; Baldwin et al. (2014) MBio. 5, e01714-01714. doi: 01710.01128/mBio.01714-01714). The raw data from the microarray images were extracted using Agilent Feature Extraction software. The R program was used for normalization and data analyses. Scale factor was calculated using the signals of green and red channels for human probes. Scale factors are the sum of green/sum of red signal ratios of human probes. Scale factors were then used to obtain normalized signals for all other probes. For all probes except human probes, normalized signal is log 2 transformed of green signals/scale factors modified red signals (log 2 g−log 2 scale factor*r). On the normalized signals, t-test is applied to select probes significantly present in test samples (virus samples) by comparing test samples versus controls and to select probes significantly present in the test samples versus the controls. The significance cut-off was log 2 fold change >1 and adjusted p value (with multiple testing corrections)<0.05. Prevalence was calculated by counting the number of test cases with hybridization signal greater than the average signal of dark corner or negative control probes, and represented as a percentage.

Analysis at the individual probe level (both for specific and conserved probes), and at the accession (for viruses) or genera (for bacteria, fungi and parasite) level, taking into account all the probes per accession or genera, were performed. Microbial detections were ranked based on their total hybridization signal (sum of hybridization signal per accession or genera) and prevalence. Conserved probe analysis was also performed in which signals from individual probes targeting conserved sequences shared by two or more organisms in a family were measured. MAT Analysis assigned detection scores derived from multiple adjacent probes that are covered per sliding window.

Probe Capture and Next Generation Sequencing: Probe Capture was performed to vailidate PathoChip results, as previously described (Banerjee et al. (2015) Sci Rep. 5:15162., 10.1038/srep15162; Banerjee et al. (2017) Sci Rep. 7, 4036. doi: 4010.1038/s41598-41017-03466-41596; Banerjee et al. (2017) Oncotarget 8, 36225-36245. doi: 36210.18632/oncotarget.16717; Baldwin et al. (2014) MBio. 5, e01714-01714. doi: 01710.01128/mBio.01714-01714). Briefly, the WTA products of the samples were pooled together for hybridization with selected biotinylated probes that were identified for microbial signatures target samples by the PathoChip screen. The targeted sequences were then captured by Streptavidin coated magnetic beads and libraries were generated for NGS. The selected probes were synthesized as 5′-biotinylated DNA oligomers (Integrated DNA Technologies, Coralville, Iowa, USA), pooled together, and hybridized to WTA pools. The capture probe pool was added separately to each of the pooled WTAs (150 ng) in 6 separate reaction mixtures (Pr1-6) containing 3 M tetra-methyl ammonium chloride, 0.1% Sarkosyl, 50 mMTris-HCl, 4 mM EDTA, pH 8.0 (1×TMAC buffer). The reaction mixtures were denatured (100° C. for 10 mins) followed by a hybridization step (60° C. for 3 hours). Streptavidin Dynabeads (Life Technologies, Carlsbad, Calif., USA) were added with continuous mixing at room temperature for 2 hours, followed by three washes of the captured bead-probe-target complexes in 0.30 M NaCl plus 0.030 M sodium citrate buffer (2×SSC) and three washes with 0.1×SSC. Captured single-stranded target DNA was eluted in Tris-EDTA and used for library preparation using Nextera XT sample preparation kit (Illumina, San Diego, Calif., USA) followed by NGS (Banerjee et al. (2015) Sci Rep. 5:15162., 10.1038/srep15162; Banerjee et al. (2017) Sci Rep. 7, 4036. doi: 4010.1038/s41598-41017-03466-41596; Banerjee et al. (2017) Oncotarget 8, 36225-36245. doi: 36210.18632/oncotarget.16717; Baldwin et al. (2014) MBio. 5, e01714-01714. doi: 01710.01128/mBio.01714-01714). The 5 libraries were examined for quality control and submitted for NGS using an Illumina MiSeq instrument with paired-end 250-nt reads. Adapters and low-quality fragments of raw reads were first removed using the Trim Galore software (http://wwwdotbioinformaticsdotbabrahamdot acdotuk/projects/trim_galore/). The processed reads were then aligned to the metagenome and the human genome using Genomic Short-read Nucleotide Alignment Program (GSNAP) (Wu et al. (2010) Bioinformatics. 26, 873-881. doi: 810.1093/bioinformatics/btq1057. Epub February 2010) with default parameters. After alignment featureCounts (Liao et al. (2014) Bioinformatics. 30, 923-930. doi: 910.1093/bioinformatics/btt1656. Epub 213 November 1013) were employed to count how many reads aligned to each of the capture probe regions. The detailed results for these capture probes were visualized in IGV 71.

Microbial Fusion Detection: Microbial genomic insertion in somatic host chromosome was determined as previously described (Banerjee et al. (2015) Sci Rep. 5:15162., 10.1038/srep15162; Banerjee et al. (2017) Sci Rep. 7, 4036. doi: 4010.1038/s41598-41017-03466-41596). Prior to fusion detection, quality control of sequenced reads was performed. The Trim Galore software (http://wwwdotbioinformaticsdotbabrahamdot acdotuk/projects/trim_galore/) was employed for quality trimming of raw reads in order to remove adapters and low-quality fragments. Virus-Clip (Ho et al. (2015) Oncotarget. 6, 20959-20963) was used to identify the virus fusion sites in the human genome. Specifically, the virus genome was used as the primary read alignment target, and reads were first aligned to the PathoChip genome. Some mapped reads contained soft-clipped segments. Soft-clipped reads were extracted from the alignment and mapped (containing sequences of potential pathogen-integrated human loci) to the human genome. Utilizing this mapping information, the exact human and pathogen integration breakpoints at single-base resolution were identified. All the integration sites were then automatically annotated with the affected human genes and their corresponding gene regions.

Some of the host genes that supported viral genomic insertions by high sequence reads were subjected to Ingenuity Pathway Analysis (IPA) (Kramer et al. (2014) Bioinformatics. 30, 523-530. doi: 510.1093/bioinformatics/btt1703. Epub 213 December 1013) that helped to combine the host genes with knowledge extracted from the literature to predict likely outcomes. IPA software provided a statistical significance of the association of those genes with the disease outcome.

PCR validations of PathoChip detections and microbial genomic insertions: PCR primers from the conserved and/or specific regions of the microorganisms detected by PathoChip screen were used for detection validations. For the validation of microbial insertions, PCR primers were designed so that the fusion junction could be amplified, one primer being designed from the microbial sequence, while the other from the affected human gene sequence. The PCR amplification reaction mixtures for each reaction contained 200-400 ng of WTA product and 20 pm each of forward and reverse primers, 300 μM of dNTPs and 2.5U of LongAmp Taq DNA polymerase (NEB). DNA was denatured at 94° C. for 3 min, followed by 30 cycles of 94° C. for 30 s, different annealing temperature for different set of primers (generally 3 to 5 degree below melting temperature) for 30 s, and 65° C. for 30 s. The amplicons were gel extracted Sanger sequenced. The electropherograms were visualized using the BioEdit program (TA, H. (1999) Nucleic Acids Symposium Series 41, 95-98) and the sequences were subjected to NCBI BLAST for identification.

The results of the experiments are now described.

### Example 1: Technology Development for Identification of SARS-CoV-2, the Causative Agent of Covid-19

The PathoChIP v4 (Banerjee et al. (2015) Sci Rep. 5:15162., 10.1038/srep15162; Baldwin et al. (2014) MBio. 5, e01714-01714. doi: 01710.01128/mBio.01714-01714) was modified to include detection of Coronaviruses, including SARS-CoV-2. A bioinformatics approach was utilized to design unique and conserved probes specific for Coronaviruses. Briefly, genomic sequences in fasta format were downloaded from the NCBI website for 3 human coronaviruses (MERS, SARS and SARS-CoV-2). A pairwise alignment between SARS and SARS-CoV-2, and SARS and MERS, was then performed using stretcher (https://wwwdotebidotacdotuk/Tools/psa/emboss stretcher/) to identify the co-linear domains of the viral agents. This identified the percentage of conserved genome that maintains the Coronaviridae family. Furthermore, the regions that are unique to each of the virus were identified. The aligned sequences were then scanned using a sliding window of 60 nucleotides and the identity score computed for each window. This methodology was then accepted as stringent since regions that had greater than 42/60 nucleotides matching were considered conserved and regions less than 24/60 nucleotides matching were considered unique sequences. All segments were then concatenated into conserved and unique sequences. The unique sequence regions were blasted against the PathoChIP database of 60 synthetic chromosomes to ensure its specificity, which showed no significant hits. These regions were selected for probe design. During this design process the probes were then screened against the human genome, mouse genome and Arabidopsis thaliana genomes to determine if any cross hybridization occured with any known genomes. This enhanced the specificity of the probes selected by reducing the possibility of signals on the arrays due to sequences from other genomes.

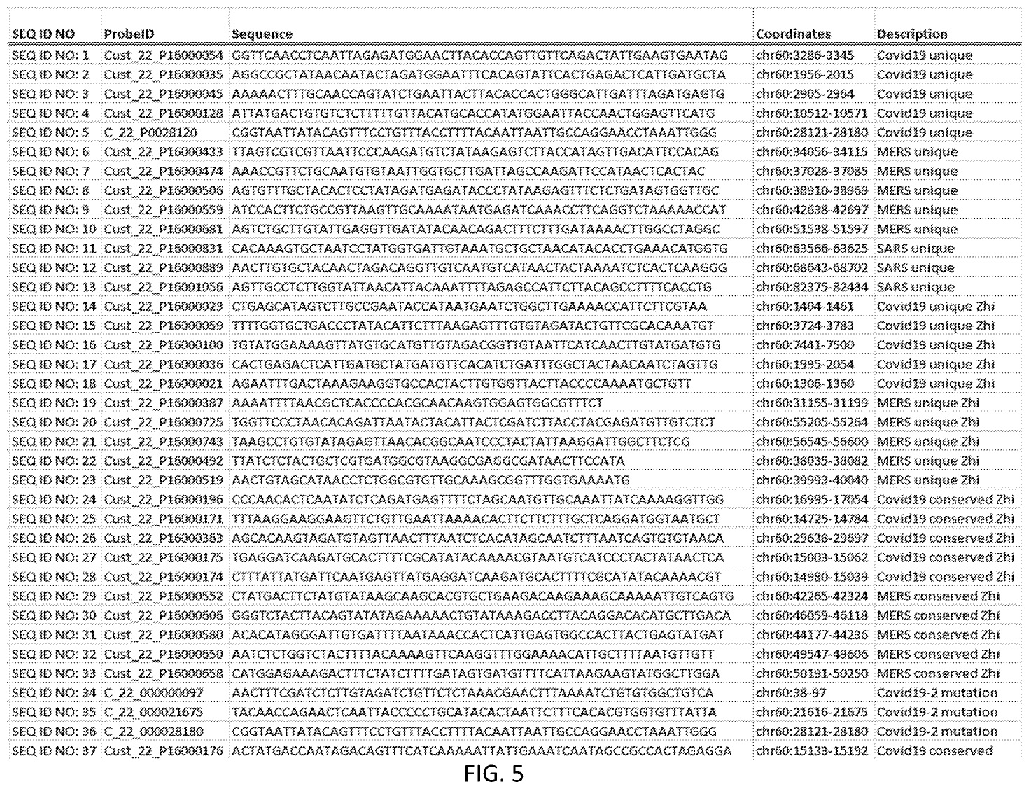

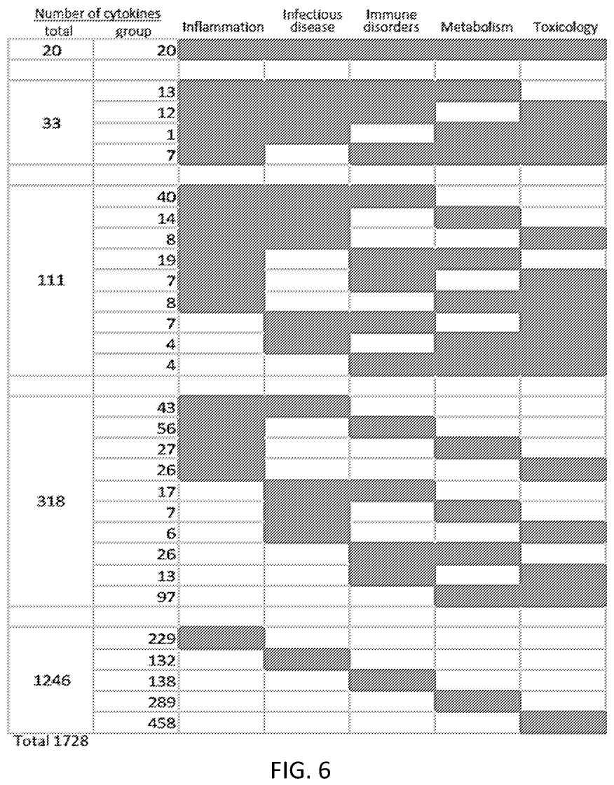

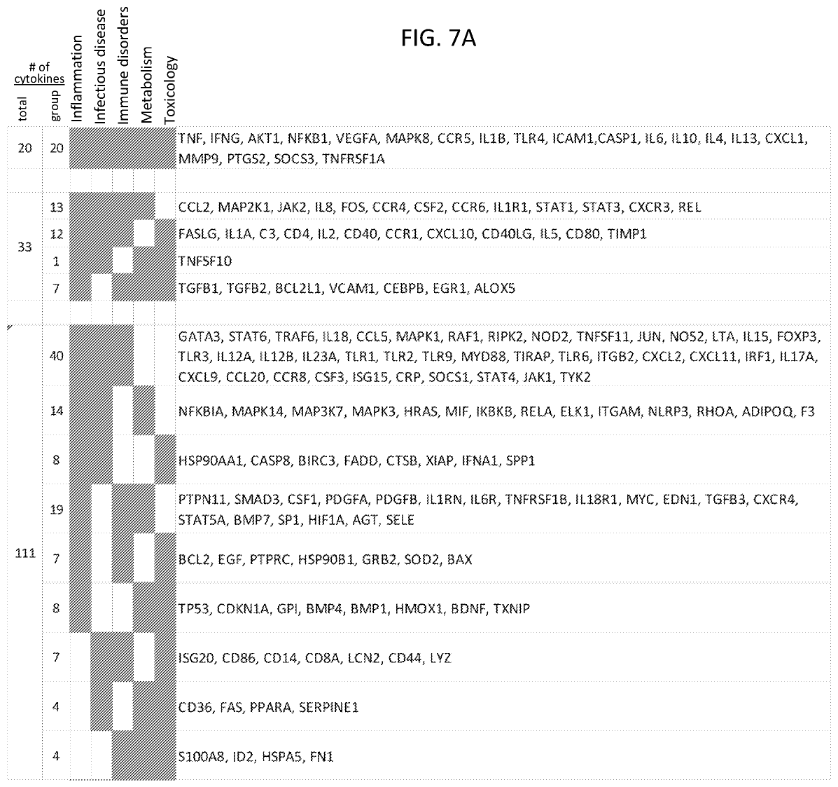

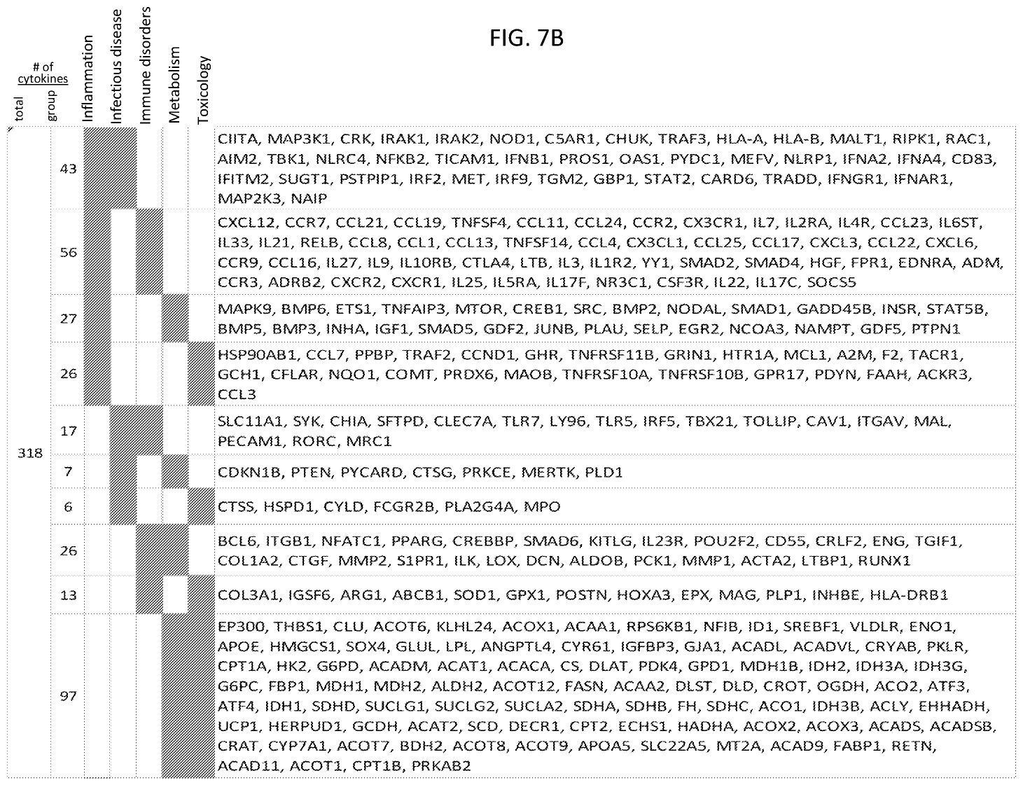

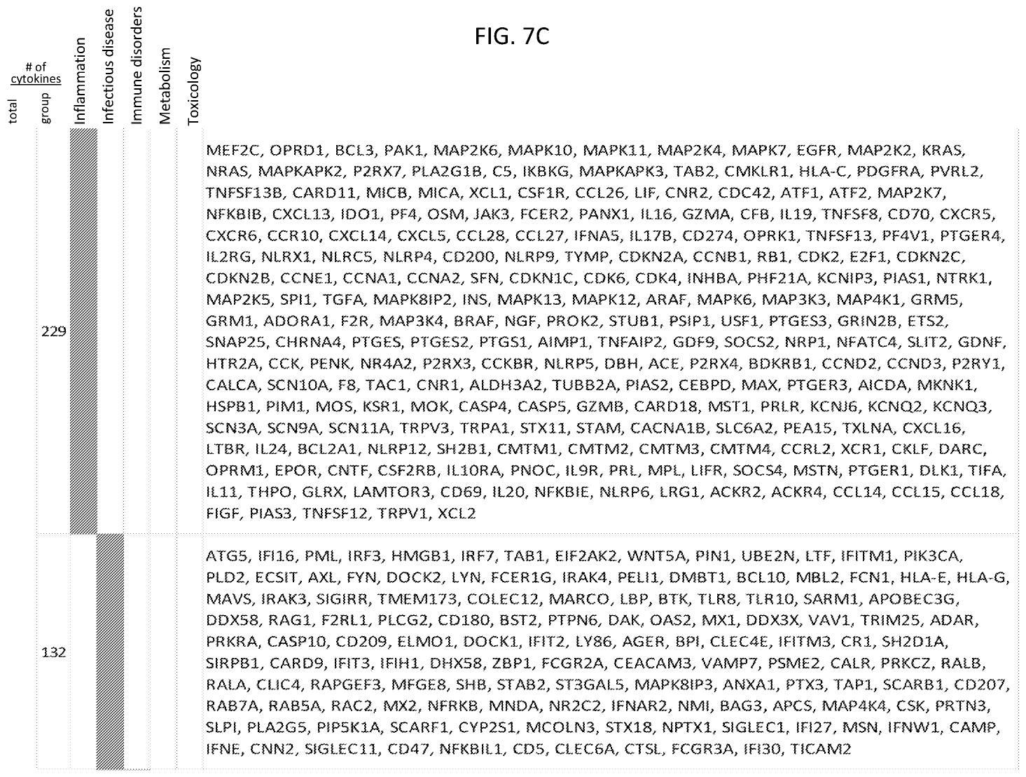

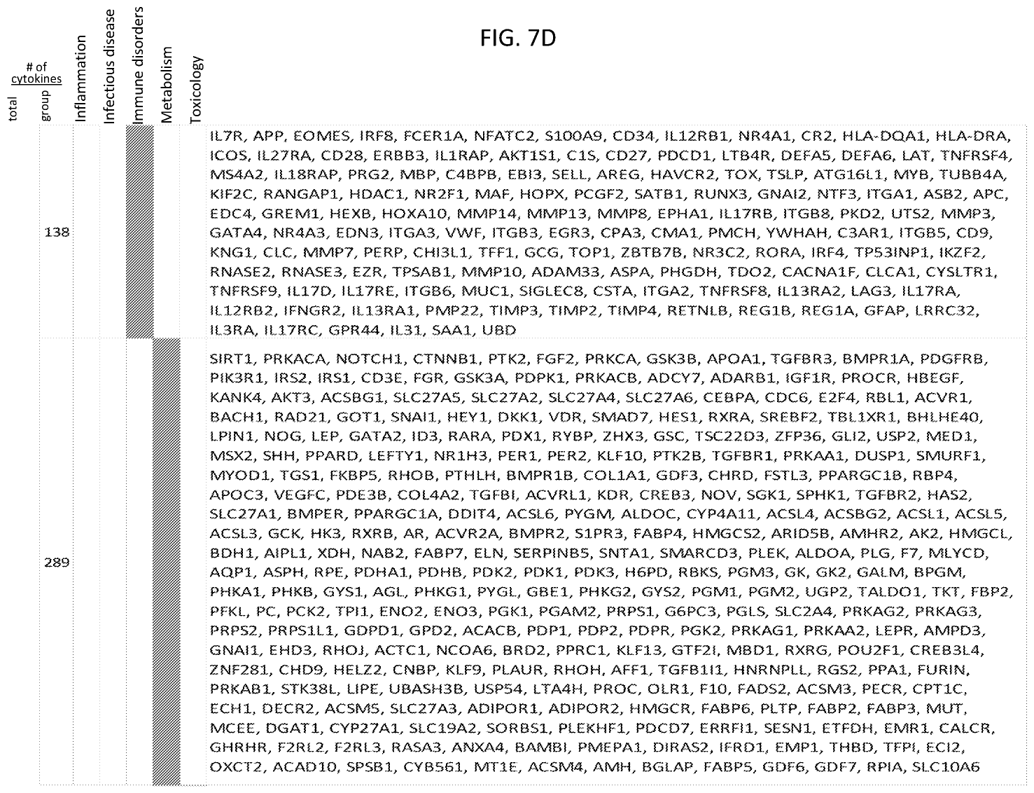

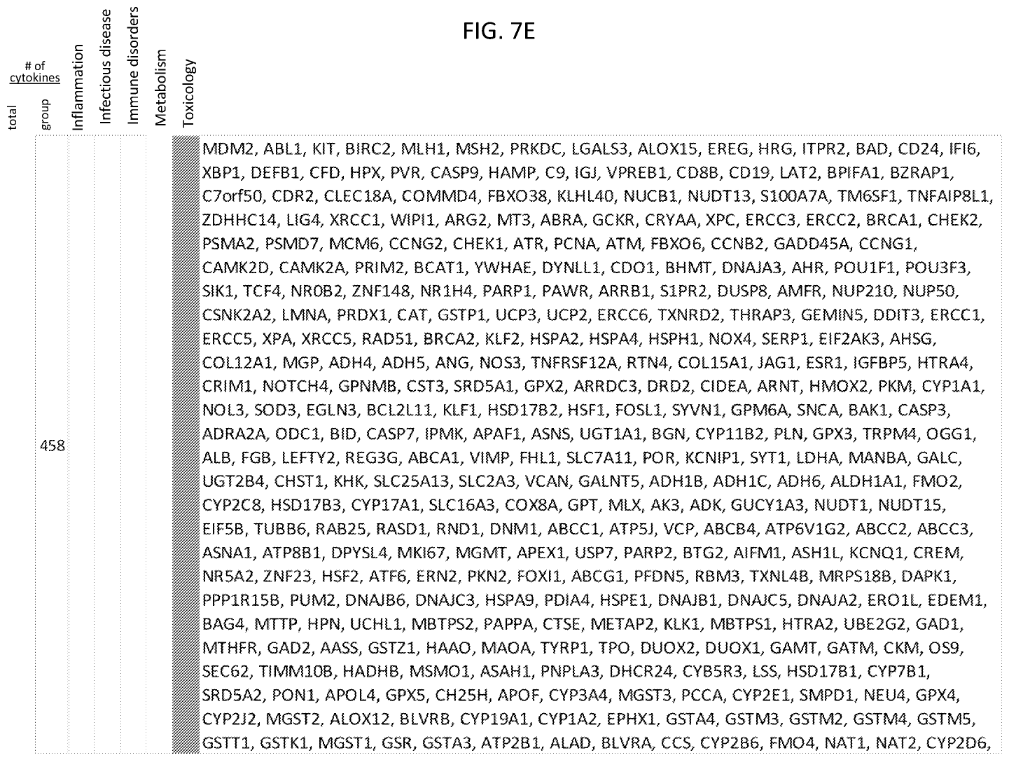

To design probes for SARS-CoV-2 and the other coronaviruses infecting humans, SARS and MERS, the selected regions were put through the Agilent CGH pipeline for probe design, which was used for the previous designs of PathoChIP v2, v3 and v4. Based on this pipeline a total of 1,147 probes were selected with probe score information on sequence homology with viral genomes, secondary structures and base composition for example. The resulting probes were also scored against the human genome to check for cross hybridization with human genome DNA to ensure that there was not any background on the array from genomic DNA. Based on this information for unique and conserved probes generated for these viruses and the strategy for selection of a minimum of 5 unique and 5 conserved probes for each pathogen, 8 unique probes for SARS-CoV-2 and 5 conserved probes were selected. Furthermore, 3 probes specifically located in SARS-CoV-2 mutation regions were also selected. Therefore, a total of 37 probes (FIG. 5, SEQ ID NOs: 1-37) were selected and added to the PathoChIP as a revised version, v5. This was in the same format as the previous v4 in an 8×60000 design format of glass slide.

### Example 2: Validation of the Assay for SARS-CoV-2

The probes were first tested for their ability to uniquely detect the SARS-CoV-2 virus. To validate the assay, samples were obtained from the NII/NIAID Biodefense and Emerging Infections (BEI) Research Resources Repository, who provided genomic RNA for SARS-CoV-2, synthetic RNA, or regions of the SARS-CoV-2 that can be used for testing specific probes or as a negative control if they belong to a region that does not contain the selected probes.

RNA and DNA was extracted from each sample. RNA was separated for reverse transcription and cDNA synthesis, then the DNA and RNA fractions were combined (50 ng each) and amplified. This was used for generation of increased pools of viral sequences. Nucleic acids were labeled with Cy5 and Cy3 fluorescent labels. Human genomic DNA was also labelled so that they both could be mixed and hybridized to an array containing SARS-CoV-2 specific probes. Samples were hybridized for 12 hours, washed, scanned, and then transferred to the informatics pipeline for extraction of the data and analyses for identification of the SARS-CoV-2 probes.

This rapid test for detection of SARS-Cov2 coronavirus has shown high sensitivity and accuracy.

### Example 3: The Immunome ChIP

Probes were engineered bioinformatically for over 1,720 immune markers to cover all innate and adaptive responses to infections, inflammatory, immune disorders, metabolism and toxicological responses (Table 1; SEQ ID NOs: 38-4671). Unique probes were generated that provide a snapshot of the immune response to SARS-CoV-2, which allows for stratification of the patients who may be at greater risk for development of disease severity. These probes can used in conjunction with the PathoChip (Immunome ChIP or ImmunoChIP), providing a test that concomitantly covers all known respiratory pathogens including other coronaviruses from bats, and other mammals, providing accurate and efficient detection of SARS-CoV-2, as well as other respiratory pathogens and cytokines associated with the Covid-19 disease. The benefits of this test include the sensitivity and accuracy from using multiple probes across the genome to avoid negative results if one probe fails in the assay screen. The overall screen can be used in a research lab setting as well as in a clinical lab.

The Immunome ChIP determines unique signature responses from each unique antigen, providing the ability to stratify patients based on disease severity due to immune response and co-infections.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

| SEQ ID | | |
|---|---|---|
| NO: | Molecule | Probe |
| SEQ ID | ref\|NM_001204402.11 | TTTCATCTTCCTTCT |
| NO: 38 | Homo sapiens phosphoribosyl | TTGGATCTATTTGGC |
| | pyrophosphate synthetase 1 (PRPS1), | CTCTCAAATGAACTG |
| | | AGATTCCTGTTAAAA |
| SEQ ID | GI 397938 Z22576 H. sapiens CD69 gene | GGGATGATCGTGTAT |
| NO: 39 | | TTATTTTTTTACTTC |
| | | CTCAGCTGTAGACAG |
| | | GTCCCTTTCGATGGT |
| SEQ ID | GI 401709925 NM_015953 | GAGTTTGAAAGAGCG |
| NO: 40 | Homo sapiens nitric oxide synthase | GGCTAAGCATATTGA |
| | interacting protein (NOSIP), transcript | ATGTGGTCTCACATT |
| | variant 1, mRNA | AAGAACAAAATCTGG |
| SEQ ID | ref\|NG_029387.2\| | CTCTATGACCTCATC |
| NO: 41 | Homo sapiens V-set pre-B cell | TAACCTTCATTAACT |
| | surrogate light chain 1 (VPREB1), | CCTTAAAGGCCCTAT |
| | | CTCCAAATACAGGCA |
| SEQ ID | GI 194353939 NG 008255 | GAAACACAGGGAGAG |
| NO: 42 | Homo sapiens solute carrier | AAACATGTTCCCAGT |
| | family 19 member 2 (SLC19A2), | GTTGATATTTGAAAG |
| | RefSeqGene on chromosome 1 | CTGATCTGTTTTGTC |
| SEQ ID | GI 283806588 NG_016341 | CATTAGGAAGGAAAG |
| NO: 43 | Homo sapiens cyclin dependent | GAACAGAAAATGAAC |
| | kinase inhibitor 1B (CDKN1B), | CAACCTGAGTGTTAG |
| | RefSeqGene on chromosome 12 | GAGACTTGCATCTAG |
| SEQ ID | GI 345842406 NM_005849 | GAAACGTTTTAATTT |
| NO: 44 | Homo sapiens immunoglobulin | TCAATGAAGTCACTG |
| | superfamily member 6 (IGSF6), | AAAATCCAACTCCAG |
| | mRNA | GAGCTATGGCAGTGT |
| SEQ ID | GI 312434026 NM_002995 | TTAGAGTCCAGCTTG |
| NO: 45 | Homo sapiens X-C motif chemokine | TAATGGTTCTTTACA |
| | ligand 1 (XCL1), mRNA | CATCAGTCACAAGTT |
| | | ACAGCTGTGACAATG |
| SEQ ID | GI 1216866539 NG_012872 | ATAGAAGAGTAGACT |
| NO: 46 | Homo sapiens gelsolin (GSN), | GCACAGTATCAGTAG |
| | RefSeqGene on chromosome 9 | TGTGACCTCCAGAGA |
| | | GTACTTAACCCTTTC |
| SEQ ID | GI 224922763 NG_011563 | GAATAAAATGGCTGG |
| NO: 47 | Homo sapiens hepcidin antimicrobial | TTCTTTTGTTTTCCA |
| | peptide (HAMP), RefSeqGene on | AACCAGAGTGTCTGT |
| | chromosome 19 | TGTCCTTTCTCTCTG |
| SEQ ID | GI 298919225 NG_023030 | GAAACATCTTGGAAA |
| NO: 48 | Homo sapiens heme oxygenase 1 | CTTCCAAAAAGACCA |
| | (HMOX1), RefSeqGene on | AAGTGCTCCACAAAA |
| | chromosome 22 | TGTACCCTCACTAAG |
| SEQ ID | GI 654825103 NG_029876 | CAAGTAGTTCCTACC |
| NO: 49 | Homo sapiens C-C motif chemokine | ACACCTGAGTTTATG |
| | ligand 23 (CCL23), RefSeqGene on | TCAATGAAGTGACTT |
| | chromosome 17 | TTGGAAAGCCTTAGG |
| SEQ ID | GI 307611978 NM_005409 | TACGCTATGGGATAC |
| NO: 50 | Homo sapiens C-X-C motif chemokine | TGGCAAAAGTGCACA |
| | ligand 11 (CXCL11), transcript | TATTTCATAACCAAA |
| | variant 1, mRNA | TTAGTAGCACCAGTC |
| SEQ ID | GI 29501801 AY260471 | CACAGACAACATTTA |
| NO: 51 | Homo sapiens BCL2-antagonist/killer 1 | TAGAACACATACTGT |
| | (BAK1) gene, complete cds | GTGCCAGGCTTTATG |
| | | CTGAGAGTTCATCAT |
| SEQ ID | GI 119703747 NM_001432 | CTCTCAGCTGATGTG |
| NO: 52 | Homo sapiens epiregulin (EREG), mRNA | TCCTGTACACAGTGG |
| | | GAAGATTTTAGTTCA |
| | | CACTTAGTCTAACTC |
| SEQ ID | GI 291463310 NM_002169 | CAACATCGAAATGAT |
| NO: 53 | Homo sapiens interferon alpha 5 | TCTCATTGACTAGTA |
| | (IFNA5), mRNA | CACCATTTCACACTT |
| | | CTTGAGTTCTGCCGT |
| SEQ ID | GI 149408121 NM_024119 | TAGACATTTTCATAT |
| NO: 54 | Homo sapiens DExH-box helicase 58 | GCACTGGATGGAGTT |
| | (DHX58), mRNA | AGGGAAACTGAGGCA |
| | | AAAGAATTTGCCATA |
| SEQ ID | GI 149408158 NM_005259 | AGAGGTTGGCTTTCA |
| NO: 55 | Homo sapiens myostatin (MSTN), | ATTCTGTAGCATACT |
| | mRNA | TGGAGAAACTGCATT |
| | | ATCTTAAAAGGCAGT |
| SEQ ID | GI 117606571 EF064760 | CTGATTCAGCGGATC |
| NO: 56 | Homo sapiens chemokine (C-C motif) | TAGAATGAGACACAA |
| | receptor 3 (CCR3) gene, complete cds | GAATGTGCTTAGCAT |
| | | CACTAGCAAGTTTTC |
| SEQ ID | GI 1036031503 AHO11685 | CAAGGGTAACCATTG |
| NO: 57 | Homo sapiens chromosome 1 AMPK | ACTTAGCAGTCTCAT |
| | beta-2 subunit (PRKAB2) gene, | TTTAGCTGTCATTTC |
| | complete cds | TGTCAAGACTGATGG |
| SEQ ID | GI 351542176 NG_030342 | GGAACGCTTCCAGCT |
| NO: 58 | Homo sapiens collectin subfamily | CTTTGAGACGGATAT |
| | member 12 (COLEC12), RefSeqGene | TGTGAACATCATTAG |
| | on chromosome 18 | CAATATCAGTTACAC |
| SEQ ID | GI 341572559 NG_029522 | CCTAACCTTCATTCT |
| NO: 59 | Homo sapiens tachykinin receptor 1 | TAGTTTTACACTAGC |
| | (TACR1), RefSeqGene on chromosome 2 | ATCGTGCTCATATGT |
| | | GCGACAAGCAAGAAG |
| SEQ ID | GI 1220516002 NM_023003 | TAGTGCCAACAGTAG |
| NO: 60 | Homo sapiens transmembrane 6 | TGAGCATGAAATGAA |
| | superfamily member 1 (TM6SF1), | ACTATTCAAAAGAGA |
| | transcript variant 1, mRNA | ATATGGCCTGTGCAT |
| SEQ ID | GI 1126528245 AF058925 | CTTTAGGGATCTAGC |
| NO: 61 | Homo sapiens Jak2 kinase mRNA, | TCTTCGAGTGGATCA |
| | complete cds | AATAAGGGATAACAT |
| | | GGCTGGATGAAAGAA |
| SEQ ID | GI 209870110 NM_013943 | CTTTAATTTGGAGAC |
| NO: 62 | Homo sapiens chloride intracellular | ACTCCACCACAAACT |
| | channel 4 (CLIC4), mRNA | TTTCACTTTAGAGGT |
| | | AGCTTGCCATCTCTC |
| SEQ ID | GI 300244555 NG_023331 | ATGGTCATTCTTCTC |
| NO: 63 | Homo sapiens matrix Gia protein | TTTTCTGCCTGAGAG |
| | (MGP), RefSeqGene on chromosome 12 | TAGGTGGAATAGGAG |
| | | ATCTGTATTACTCCA |
| SEQ ID | GI 401709984 NG_032897 | AATCAAGAGCCAAAG |
| NO: 64 | Homo sapiens platelet factor 4 | TCATTCTTTCTCAGA |
| | (PF4), RefSeqGene on chromosome 4 | GAAGGGAAGACTGTC |
| | | CATATGGATCTCTTC |
| SEQ ID | GI 217035112 NM_152672 | CTCATACTGGAGACT |
| NO: 65 | Homo sapiens solute carrier | TTTCTAATGACTGTG |
| | family 51 alpha subunit (SLC51A), | CTGACACGAATGTAC |
| | mRNA | TACCGAAGGAAAGAC |
| SEQ ID | GI 255958254 NG_012690 | GACAAGAGTTGTAAC |
| NO: 66 | Homo sapiens flavin containing | TTGGGCTCTGAAGAA |
| | monooxygenase 3 (FMO3), RefSeqGene | AGGAAACAAGGCAAT |
| | on chromosome 1 | TTATGGGGAAAATTG |
| SEQ ID | GI 669033292 NM_006798 | AATTTCCATATACCA |
| NO: 67 | Homo sapiens UDP | AACAAATCAGGGAGC |
| | glucuronosyltransferase family 2 | CACCGTAGGAGAGTA |
| | member A1 complex locus (UGT2A1), | GTGTGTTATGAGAAA |
| | transcript varian [symbol] | |
| SEQ ID | GI 24286757 AY163769 | TTCATAAGAAAGGCA |
| NO: 68 | Homo sapiens excision repair cross- | TTGGTATCACCAAAG |
| | complementing rodent repair deficiency, | CACTTTGCAAGAGAG |
| | complementation group 3 (xeroc [symbol] | GCTTAGGGTTATATG |
| SEQ ID | GI 238018066 NG_012196 | CTATACAACAACCAC |
| NO: 69 | Homo sapiens fibronectin 1 (FN1), | AATTACACTGATTGC |
| | RefSeqGene on chromosome 2 | ACTTCTGAGGGCAGA |
| | | AGAGACAACATGAAG |
| SEQ ID | GI 793956 L41915 | EATGCTATGTCAAGA |
| NO: 70 | Homo sapiens retinoblastoma | CTGTTGAAGAAGTAT |
| | susceptibility protein (RB1) | GATGTATTGTTTGCA |
| | gene from tumor GOS537, exon 4, | CTCCCAGCAAATTGG |
| | (complete) bases 4179 [symbol] | A |
| SEQ ID | GI 225735577 NG_011719 | GGTAAGGTGACCAAG |
| NO: 71 | Homo sapiens integrin subunit | AGCTCTAGTTTGCTT |
| | alpha M (ITGAM), RefSeqGene on | AACTCTGTCCCAGAT |
| | chromosome 16 | TTAGTACTGAAATCC |
| SEQ ID | GI 543583730 NM_006601 | TTTTACATCTCTTAG |
| NO: 72 | Homo sapiens prostaglandin E | CATGGAGGACGAAGA |
| | synthase 3 (PTGES3), transcript | AAGCTGTACATTGTT |
| | variant 1, mRNA | GCTTGAGAGTCTGTA |
| SEQ ID | GI 225903414 NG 011740 | CAATTTCCTGTCCAA |
| NO: 73 | Homo sapiens matrix metallopeptidase 1 | TCACAGATGGTCACA |
| | (MMP1), RefSeqGene on chromosome 11 | TGCTGCTTTCCTGAG |
| | | TTAACCTATTAACTC |
| SEQ ID | GI 342837710 NG_029639 | TTTAGAGAGTTTTGG |
| NO: 74 | Homo sapiens inhibitor of DNA binding 1, | TGTGTGTGGTAAGGG |
| | HLH protein (ID1), RefSeqGene on | GATGAGGAGTCTGGC |
| | chromosome 20 | TAATTAATTGATGAT |
| SEQ ID | GI 31096355 AY300794 | AGTGGAGGATTTAGG |
| NO: 75 | Homo sapiens PRKC, apoptosis, WT1, | ACTAAGGGATTGCTA |
| | regulator (PAWR) gene, complete cds | AGGAAGGAAAATAAG |
| | | GAGTCTTGCTCTTTG |
| SEQ ID | GI 343790995 NG_029773 | GAAAGAGCATATGAA |
| NO: 76 | Homo sapiens activating transcription | GGCAGTTAAGTAAGG |
| | factor 6 (ATF6), RefSeqGene on | CAGGAAAGAGCTTTA |
| | chromosome 1 | TTTGAGGCACTGAAA |
| SEQ ID | GI 22538815 NM_005623 | CCAAATATTTCAAAA |
| NO: 77 | Homo sapiens C-C motif chemokine | TCTGAAGCCATGAGC |
| | ligand 8 (CCL8), mRNA | CTTCATACATGGACT |
| | | GAGAGTCAGAGCTTG |
| SEQ ID | GI 1005261235 NM_000399 | AGGTTCTTCCTAAAA |
| NO: 78 | Homo sapiens early growth response 2 | CTTAGCCCATTCTAG |
| | (EGR2), transcript variant 1, mRNA | TCTCTCTTAGGTGAG |
| | | TTGACTATCAACCCA |
| SEQ ID | GI 40787172 AY513239 | AATTACCAATGGGAA |
| NO: 79 | Homo sapiens complement component 3 | AGCTAAGGTGAGTTC |
| | (C3) gene, complete cds | CAAGTTTGGCTTCAC |
| | | CAGAGAAAACTAATT |
| SEQ ID | GI 692314725 NM_002416 | ACCACTTTATTTCTT |
| NO: 80 | Homo sapiens C-X-C motif chemokine | TTTGTTCCCCTTTGC |
| | ligand 9 (CXCL9), mRNA | TTCATTCAAGTCAGC |
| | | TCTTCTCCATCCTAC |
| SEQ ID | GI 954169997 NM_002177 | TTTGCCATATAACAC |
| NO: 81 | Homo sapiens interferon omega 1 | TTGCACATGTGACTC |
| | (IFNW1), mRNA | TGGTCAATTCAAAAG |
| | | ACTCTTATTTCGGCT |
| SEQ ID | GI 41393574 NM_033214 | GTAGAGCTCTTTTTG |
| NO: 82 | Homo sapiens glycerol kinase 2 | GTACCATTGATTCAT |
| | (GK2), mRNA | GGCTTATCTGGAGTT |
| | | TGACAGGAGGAGTTA |
| SEQ ID | GI 313665837 NM_152230 | GTATTACAACTGTGA |
| NO: 83 | Homo sapiens inositol polyphosphate | ACCAAACGGATAAGG |
| | multikinase (IPMK), mRNA | CCCTAACCATTTTCA |
| | | GCATTCTCTTTGGAT |
| SEQ ID | GI 735661405 NM_002201 | CTTATTCTTCTCTCC |
| NO: 84 | Homo sapiens interferon stimulated | AAGCTGGGTTAACAG |
| | exonuclease gene 20 (ISG20), | TAGACAGGACCCATT |
| | transcript variant 1, mRNA | TCTGTGTGATGTTAG |
| SEQ ID | GI 219879808 NM_000922 | AAGTCATTTCCTCTC |
| NO: 85 | Homo sapiens phosphodiesterase | TACGGAGTATTAGTA |
| | 3B (PDE3B), mRNA | GCTTAATGGGTGCTT |
| | | TCTCAGGTTCCTGTA |
| SEQ ID | GI 209977034 NM_021995 | AGAAACGTGAGACTC |
| NO: 86 | Homo sapiens urotensin 2 (UTS2), | CTGATTGCTTCTGGA |
| | transcript variant 1, mRNA | AATACTGTGTCTGAA |
| | | GTGAAATAAGCATCT |
| SEQ ID | GI 1024846802 NG_046996 | TCAGATATGTTCTAT |
| NO: 87 | Homo sapiens mediator complex | GTGGAAGTGCAGTTA |
| | subunit 1 (MED1), RefSeqGene on | GATCCTGCAGGACAG |
| | chromosome 17 | CTTTGTGATGTAAAA |
| SEQ ID | GI 590122055 NM_021649 | AGTCCAGAATCTGCT |
| NO: 88 | Homo sapiens toll like receptor | ACAAGATGACTTTGG |
| | adaptor molecule 2 (TICAM2), mRNA | TATCAAACCCGGAAT |
| | | AATCTTTGCTGAGAT |
| SEQ ID | GI 32165625 AY323826 | TAGATTCAGGAGATT |
| NO: 89 | Homo sapiens interleukin 10 | GTATTTCCCTAGCAT |
| | receptor, beta (IL10RB) gene, | CCAACAGGCATGTCC |
| | complete cds | AAATGTCATCTCTTT |
| SEQ ID | GI 291045457 NG_016717 | GTTTCCTTTATTGCA |
| NO: 90 | Homo sapiens phosphoribosyl | GTCTTTGAGCTCTGT |
| | pyrophosphate synthetase 2 (PRPS2), | TTCCTATTCTGAAAG |
| | RefSeqGene on chromosome X | CATAATGCCCAGGAA |
| SEQ ID | GI 166158920 NM_013996 | AAAGAAAAAAAATCA |
| NO: 91 | Homo sapiens tachykinin precursor 1 | TTGCTTTGAAGCAGT |
| | (TAC1), transcript variant alpha, | TGTGTCAGCTACTGC |
| | mRNA | GGAAAAGGAAGGAAA |
| SEQ ID | GI 78482607 NM_021129 | TTTCACATGGTAGTT |
| NO: 92 | Homo sapiens pyrophosphatase | GAAGTACCACGCTGG |
| | (inorganic) 1 (PPA1), mRNA | TCTAATGCAAAAATG |
| | | GAGATTGCTACAAAG |
| SEQ ID | GI 212549700 NG_008949 | CTGTGGTGTTAGTTA |
| NO: 93 | Homo sapiens apolipoprotein C3 | CTTCTGAGAGGACAG |
| | (APOC3), RefSeqGene on chromosome 11 | CTTGCTGCCAGAGCT |
| | | CTATTTTTTATGTTA |
| SEQ ID | GI 340805888 NG_029485 | ATGAGAATATGCAGG |
| NO: 94 | Homo sapiens ATPase plasma | TCATACTGGGGAGGG |
| | membrane Ca2+ transporting 1 | GAATATCTGGTTGTA |
| | (ATP2B1), RefSeqGene on chromosome 12 | TAGTTCAGCTGAATA |
| SEQ ID | GI 1028224151 NG_008493 | CTGAAGACACTGGGT |
| NO: 95 | Homo sapiens estrogen receptor 1 | ACATCTGTTATGGTC |
| | (ESR1), RefSeqGene on chromosome 6 | CCTGTGTACACTCGG |
| | | ATATTCTAAATGAGA |
| SEQ ID | GI 442092991JQ924061 | CTTCGAGATGTGTGG |
| NO: 96 | Homo sapiens ATG5 (ATG5) mRNA, | TTTGGACGAATTCCA |
| | complete cds | ACTTGTTTCACGCTA |
| | | TATCAGGATGAGATA |
| SEQ ID | GI 261823945 NG_013238 | CTTTTGTTTATGAGT |
| NO: 97 | Homo sapiens interleukin 9 receptor | GTGCATGCAAGTGTG |
| | (IL9R), RefSeqGene on chromosome | ATGAGTGTGAAAGTG |
| | X and Y | TTCCTGTAGACATGT |
| SEQ ID | GI 295842487 NM_014470 | TCTGAACTCATCTCT |
| NO: 98 | Homo sapiens Rho family GTPase 1 | TCTACCTTCAAGAAG |
| | (RND1), mRNA | GAAAAGGCCAAAAGC |
| | | TGTTCCATTATGTGA |
| SEQ ID | GI 224809340 NG_011507 | ACACCAAATGTTACA |
| NO: 99 | Homo sapiens glutamate ionotropic | TTAGCACACACTCAT |
| | receptor NMDAtype subunit 1 (GRIN1), | GCACCTCAGCATCAC |
| | RefSeqGene on chromosome [symbol] | ACAATCAATTTCATA |
| SEQ ID | GI 68160936 NM_005082 | CAAATGTGCAGCTCA |
| NO: 100 | Homo sapiens tripartite motif | GGATAGGGAAGCATA |
| | containing 25 (TRIM25), mRNA | AGACCCTCTGTTTAA |
| | | AAGAGAGTCAAGTAG |
| SEQ ID | GI 589269250 NG_007475 | TCCTCTCCACTCTTT |
| NO: 101 | Homo sapiens aquaporin 1 | CAGTAACTGATTCAG |
| | (Colton blood group) (AQP1), | ACTTGATGAGTCTTT |
| | RefSeqGene on chromosome 7 | CCCCTTTCTCTCATC |
| SEQ ID | GI 299829203 NG_023197 | CAATATGCTACAAAG |
| NO: 102 | Homo sapiens parathyroid | GGCTTCACCACTCCT |
| | hormone like hormone (PTHLH), | AGAGAAGAAACTGTC |
| | RefSeqGene on chromosome 12 | CAATTTTCCTGAGAT |
| SEQ ID | GI 223972657 NG_009817 | ATTTTGTTTATTTCA |
| NO: 103 | Homo sapiens E1A binding protein | ATTGCTGAACTGGCT |
| | p300 (EP300), RefSeqGene on | GAGCTTTTACTCTCC |
| | chromosome 22 | CCACCTTAGGTTGTT |
| SEQ ID | GI 83699682 DQ314888 | GCATCTGTGTGAAAT |
| NO: 104 | Homo sapiens v-rel | CTCAGGATATAGTGT |
| | reticuloendotheliosis viral | CCACTAAACTTCAGT |
| | oncogene homolog (avian) (REL) | GCACTGCCTAATGAG |
| | gene, complete cds | |
| SEQ ID | GI 225735573 NG_011718 | GTATCAAACACACTC |
| NO: 105 | Homo sapiens alcohol | CTCTGGCTCCTAGCT |
| | dehydrogenase 1C (class 1), | CTCTCTGTTCCACTT |
| | gamma polypeptide (ADHIC), | TGTTTATCCAATTTC |
| | RefSeqGene on chromosome [symbol] | |
| SEQ ID | GI 359338962 NG_031836 | ATTTAACCTGTGCAA |
| NO: 106 | Homo sapiens Z-DNA binding | CAATCCCACAAGGTA |
| | protein 1 (ZBP1), RefSeqGene | AGTACTTCTATGACC |
| | on chromosome 20 | CCCTTTACAAAGACA |
| SEQ ID | GI 28932927 AY245248 | CTTTGAACTAGGCCA |
| NO: 107 | Homo sapiens BCL2-interacting | TTAGATTTCCGTGCC |
| | killer (apoptosis-inducing) | ACAGTCATCGTCATT |
| | (BIK) gene, complete cds | ATCGTCATCTACATC |
| SEQ ID | GI 1028908919 NG_050629 | GATTCTATATTGCAG |
| NO: 108 | Homo sapiens neurotrophin 3 (NTF3), | GACCACCAACTTGGA |
| | RefSeqGene on chromosome 12 | ATTTCAGCTCAGTTC |
| | | AGCAAACACTTTTGA |
| SEQ ID | GI 33946335 NM_002166 | TCAAGTGTGTTTATT |
| NO: 109 | Homo sapiens inhibitor of DNA | GAATGGTGATTGCCT |
| | binding 2, HLH protein (ID2), mRNA | GCTTTATTTCAGAGG |
| | | ACCAGTGCTTTGATT |
| SEQ ID | GI 315434256 NM_001926 | ATGACCTAGAAGGAA |
| NO: 110 | Homo sapiens defensin alpha 6 | ACTGTCGTGTGTCCT |
| | (DEFA6), mRNA | ATACATTGCCATCAA |
| | | CTTTGTTTCCTCATC |
| SEQ ID | GI 326537295 NG_028240 | GGCATTGAACAAAGC |
| NO: 111 | Homo sapiens cell division cycle 6 | TAAAAACTCCAGTGA |
| | (CDC6), RefSeqGene on chromosome 17 | TGCCAAACTAGAACC |
| | | AACAAATGTCCAAAC |
| SEQ ID | GI 117606551 EF064750 | CACAATTTGAGAAGC |
| NO: 112 | Homo sapiens CD80 molecule (CD80) | AAGGAATTAAGCTCT |
| | gene, complete cds | CATTTGGCTAGAGTG |
| | | ACCAGGTATCCTCTT |
| SEQ ID | GI 1064244211 NG_051562 | GAGATTCCTATTTTG |
| NO: 113 | Homo sapiens ChaC glutathione | AGGGTCAAGGTTGAA |
| | specific gamma- | CTTGTGTTACACAGG |
| | glutamylcyclotransferase 1 | ACATGGGATATAGCC |
| | (CHAC1), RefSeqGene on chro [symbol] | |
| SEQ ID | GI 33413399 NM_001984 | CCTGATAACTTCATA |
| NO: 114 | Homo sapiens esterase D (ESD), | GCTGCCTGTACAGAA |
| | mRNA | AAGAAAATCCCCGTT |
| | | GTTTTTCGATTGCAA |
| SEQ ID | GI 389565481 NM_002054 | TAGAAGAACTGGTAG |
| NO: 115 | Homo sapiens glucagon (GCG), | CCACAGTGGTGAAAT |
| | mRNA | TGGAAAGAGAACTTT |
| | | CTTCCTGAAACCTTT |
| SEQ ID | GI 296080750 NM_002704 | GGCAGGTGATGAATC |
| NO: 116 | Homo sapiens pro-platelet basic | TGCTGATTAATTTGT |
| | protein (PPBP), mRNA | TCTGTTTCTGCCAAA |
| | | CTTCTTTAACTCCCA |
| SEQ ID | GI 32187524 AY324387 | ATCCTAGGCAAGCAA |
| NO: 117 | Homo sapiens glutathione S-transferase | TGACACTACTGTATT |
| | pi (GSTP1) gene, complete cds | AGGATTCTCCAGAGA |
| | | AGCTAAGCCAGTAAG |
| SEQ ID | GI 685844874 NM_002988 | TTATCATCCTTTCCC |
| NO: 118 | Homo sapiens C-C motif chemokine | CTTTCCCTTCAACTC |
| | ligand 18 (CCL18), mRNA | TTCGTACATTCAATG |
| | | CATGGATCAATCAGT |
| SEQ ID | GI 224809244 NG_011449 | GGTAACTCCTCCTTA |
| NO: 119 | Homo sapiens 3-hydroxy-3- | CTCGATACTTCATCA |
| | methylglutaryl-CoA reductase (HMGCR), | GTACTGGTGACACAG |
| | RefSeqGene on chromosome 5 | GAACCTGAAATTGAA |
| SEQ ID | GI 293597503 NG_016969 | CTGTTAGCCAAAGAT |
| NO: 120 | Homo sapiens solute carrier family | CCAGGAATCCTTCTT |
| | 6 member 2 (SLC6A2), RefSeqGene on | TCAACACAAGATCTT |
| | chromosome 16 | AGGCAGCTGAAGTAA |
| SEQ ID | GI 387849236 NM_002613 | AACTCCTTTGAACTG |
| NO: 121 | Homo sapiens 3-phosphoinositide | GACTTACAGTTTTCC |
| | dependent protein kinase 1 (PDPK1), | GAAGATGAGAAGAGG |
| | transcript variant 1, mRNA | TTGTTGTTGGAGAAG |
| SEQ ID | GI 1403722 U60524 Human aryl | TGCACTGCTACATTG |
| NO: 122 | hydrocarbon receptor (AhR) gene, | TGGGATTTATTTCTG |
| | 3′ end of exon 11 and 3′ flanking | GATGATGTGCACATC |
| | region | TAAGGATATGGATGT |
| SEQ ID | GI 1168720186 NG_053029 | ATAACTGGTTTTTCA |
| NO: 123 | Homo sapiens solute carrier family | TCCTGGACATCTGAT |
| | 27 member 3 (SLC27A3), RefSeqGene | CTCTGTGATCCAAAG |
| | on chromosome 1 | CTTCTGAACCTCAAC |
| SEQ ID | GI 166706902 NM_002053 | AAAAATGTTGCAGGA |
| NO: 124 | Homo sapiens guanylate binding | AATGCAAAGAAAGAA |
| | protein 1 (GBP1), mRNA | TGAGCAGATGATGGA |
| | | ACAGAAGGAGAGGAG |
| SEQ ID | ref\|NG_033066.2\| | TTTTAACACCCTTAT |
| NO: 125 | Homo sapiens C-C motif chemokine | GAACTAGGGACTGTT |
| | ligand 4 (CCL4), RefSeqGene | GCACCGAGTTTCACA |
| | | GTTAAGGAAACAGAG |
| SEQ ID | GI 237858685 NG_012086 | CAGGATTCTTGTGTG |
| NO: 126 | Homo sapiens interleukin 4 receptor | GATTCGGTGAGGTAA |
| | (IL4R), RefSeqGene on chromosome 16 | TACTTCTAAAGCACC |
| | | TAGCAAAGTGTCAAG |
| SEQ ID | GI 83699682 DQ314888 | GAATGTTGTTCTCCC |
| NO: 127 | Homo sapiens v-rel reticuloendotheliosis | AGAAAAATGCATAGT |
| | viral oncogene homolog (avian) (REL) | TCACTCTTACATCCT |
| | gene, complete cds | TCAGGTGTCACTCCA |
| SEQ ID | GI 1159261125 NM_001349413 | GTATTTTCAGAGAGG |
| NO: 128 | Homo sapiens kelch like family member | AGAGCTTGGTGTTTT |
| | 24 (KLHL24), transcript variant 1, mRNA | TGTGGTGCCAAGTGG |
| | | TAAGATAATGTCTCT |
| SEQ ID | GI 307775406 NM_015364 | CTGTTTTCTTCCATA |
| NO: 129 | Homo sapiens lymphocyte antigen 96 | TTTACTGAAGCTCAG |
| | (LY96), transcript variant 1, mRNA | AAGCAGTATTGGGTC |
| | | TGCAACTCATCCGAT |
| SEQ ID | GI 375151559 NM_005651 | TTTTCCATTCTGGAG |
| NO: 130 | Homo sapiens tryptophan 2,3-dioxygenase | ACGATGACAGCCTTG |
| | (TDO2), mRNA | GACTTCAATGACTTC |
| | | AGAGAGTACTTATCT |
| SEQ ID | GI 299758475 NM_001190737 | AGCAGAATCTTGGAT |
| NO: 131 | Homo sapiens nuclear factor 1 B (NFIB), | TTGTGACTTGTAGCT |
| | transcript variant 1, mRNA | TCATAAGGACTCAAC |
| | | GAAAGAGATTGCACA |
| SEQ ID | GI 18141132 AF424815 | TTGGACTTAGATCAT |
| NO: 132 | Homo sapiens SH2 domain containing SOCS | TCCTGTGGGCATCGA |
| | box protein SOCS4 mRNA, complete cds | TTTTTAGGCCGATCT |
| | | CTTAAACAGAAACTG |
| SEQ ID | GI 323098322 NM_005624 | TGGTCAGTTCAAGGA |
| NO: 133 | Homo sapiens C-C motif chemokine ligand | TGCCCCTCCCAGGCT |
| | 25 (CCL25), transcript variant 1, mRNA | ATGCTTTTCTATAAC |
| | | TTTTAAATAAACCTT |
| SEQ ID | GI 401709987 NG_032900 | ATAATCTACGGACAT |
| NO: 134 | Homo sapiens prostaglandin-endoperoxide | CTTTGAGCCTCATTT |
| | synthase 1 (PTGS1), RefSeqGene on | TCCTCTTTGCACAAT |
| | chromosome 9 | GGGGATCATGATACC |
| SEQ ID | GI 429535871 NG_033022 | ATGGGTGTGAATATA |
| NO: 135 | Homo sapiens interleukin 12A (IL12A), | CTTAATCTGGTCAAG |
| | RefSeqGene on chromosome 3 | GAATGCCAGAATACC |
| | | CTTCAAAACAGCCAT |
| SEQ ID | GI 1036029829 AH003667 | CTGTGGGAACTTTAA |
| NO: 136 | Homo sapiens chromosome 1 leptin receptor | CTTTTGGGCTTTATT |
| | (LEPR) gene, complete cds | TTTCACAGCTCTCGT |
| | | GGCATTATCCTTCAG |
| SEQ ID | GI 315365058 HQ38416O | GTGCTCACATTTTAT |
| NO: 137 | Homo sapiens fatty acid binding protein 4, | ACCCGCTCTGTCTTA |
| | adipocyte (FABP4) gene, complete cds | GTGCGTTGCAAGAGA |
| | | GGAGTATATACAGTA |
| SEQ ID | GI 269954682 NG_015989 | CATGCACCACATCCT |
| NO: 138 | Homo sapiens C-C motif chemokine ligand | CACTAACGTTTGATG |
| | 26 (CCL26), RefSeqGene on chromosome 7 | TTTTCTGTCTTTTTC |
| | | CATTGTAGCCATCTG |
| SEQ ID | GI 432134281 NG_033037 | TTCTCCTTCCTCTTC |
| NO: 139 | Homo sapiens ancient ubiquitous protein 1 | TAGTTCCTGGCCATT |
| | (AUP1); and HtrA serine peptidase 2 | TTCTATCCAAGATGT |
| | (HTRA2), RefSeqGene on c [symbol] | GGTACAACCTCTTAC |
| SEQ ID | GI 239787846 NG_012306 | GTCATTGGAGGTGGG |
| NO: 140 | Homo sapiens interferon regulatory | TTGAGTTGGTTGATG |
| | factor 5 (IRF5), RefSeqGene on chromosome 7 | GGATTTCTCTAAAGA |
| | | TATTGAGAAACTGCT |
| SEQ ID | GI 225703119 NG_011705 | GATATCTGCACGGAT |
| NO: 141 | Homo sapiens tyrosinase related protein 1 | GACTTGATGGGATCC |
| | (TYRP1), RefSeqGene on chromosome 9 | AGAAGCAACTTTGAT |
| | | TCCACTCTAATAAGC |
| SEQ ID | GI 1036032792 AH002827 | AGGCTTGAGGTAGAG |
| NO: 142 | Homo sapiens hemopexin (HPX) gene, | ACTGGGACAAGCATA |
| | complete cds | TCCAACTCTGTATTT |
| | | ATTACCATCCTTTGT |
| SEQ ID | GI 206725542 NG_008631 | TTCCTCACAGGGTTG |
| NO: 143 | Homo sapiens patatin like phospholipase | ATATGAGGATGAAAC |
| | domain containing 3 (PNPLA3), RefSeqGene | AAGATGATAGATCAT |
| | on chromosome 22 | GGTGGCATGTAGTCT |
| SEQ ID | GI 1027250680 NG_047105 | CCTAGAGAATCATCT |
| NO: 144 | Homo sapiens slit guidance ligand 2 | CAAAGTCAAAGCTAT |
| | (SLIT2), RefSeqGene on chromosome 4 | GTCCAGAAGCAGGTC |
| | | AGGTTTCAGTATCCT |
| SEQ ID | GI 28178835 NM_005530 | AAGAATTGAGGCTGC |
| NO: 145 | Homo sapiens isocitrate dehydrogenase 3 | GTGTTTTGCTACAAT |
| | (NAD(+)) alpha (IDH3A), mRNA | TAAGGACGGAAAGAG |
| | | CTTGACAAAAGATTT |
| SEQ ID | GI 238018066 NG_012196 | CTTTAAACTGTTGTG |
| NO: 146 | Homo sapiens fibronectin 1 (FN1), | CCAGTGCTTAGGCTT |
| | RefSeqGene on chromosome 2 | TGGAAGTGGTCATTT |
| | | CAGATGTGATTCATC |
| SEQ ID | GI 269954730 NG_016009 | TCTCTCTCCCAAAAT |
| NO: 147 | Homo sapiens kininogen 1 (KNG1), | AAACTACTCCCTCTT |
| | RefSeqGene on chromosome 3 | TGGTGAAACAACTGA |
| | | AATCAGGAGGACAGA |
| SEQ ID | GI 1057867548 NM_020661 | AATATTTGGGAGAGA |
| NO: 148 | Homo sapiens activation induced | AGGATGGGGAAGCAT |
| | cytidine deaminase (AICDA), | TGCAAGGAAATTGTG |
| | transcript variant 1, mRNA | CTTTATCCAACAAAA |
| SEQ ID | GI 307611978 NM_005409 | AAGCTACCTTAAGAA |
| NO: 149 | Homo sapiens C-X-C motif | AGGCTGGTTACCATC |
| | chemokine ligand 11 (CXCL11), | GGAGTTTACAAAGTG |
| | transcript variant 1, mRNA | CTTTCACGTTCTTAC |
| SEQ ID | GI 46361513 AY594694 | ATGGGACATTTTTGC |
| NO: 150 | Homo sapiens interferon gamma | TACATAACCAGAGAT |
| | receptor 1 (IFNGR1) gene, | GTGCCAGTCCCTTTC |
| | complete cds | CTGGTATAAAAAGAG |
| SEQ ID | GI 747811824 NM_017594 | CAAGCAGGATTGTTC |
| NO: 151 | Homo sapiens DIRAS family GTPase 2 | TGTTCTAAAAAGCTG |
| | (DIRAS2), mRNA | TTGCACAGCATTCGC |
| | | AATGAGATCTTTAGT |
| SEQ ID | GI 1049010774 AH002843 | ACACTTCACAAACTG |
| NO: 152 | Homo sapiens interleukin 2 receptor | AGTGAATCCCAGCTA |
| | gamma chain (IL2RG) gene, complete cds | GAACTGAACTGGAAC |
| | | AACAGATTCTTGAAC |
| SEQ ID | GI 344313160 NG_029811 | CATGAACAAATCTCT |
| NO: 153 | Homo sapiens carbohydrate | TAACTTCTCTGAGCC |
| | sulfotransferase 1 (CHST1), | TCAGTCCCTCACCTG |
| | RefSeqGene on chromosome 11 | TGAAATTGATTCAGC |
| SEQ ID | GI 11342663 NM_002463 | AAAGACTTACCACTG |
| NO: 154 | Homo sapiens MX dynamin like | AACTCATCATGCATA |
| | GTPase 2 (MX2), mRNA | TCCAAAAATCGCTCC |
| | | CGTTGTTAGAAGGAC |
| SEQ ID | GI 27501939 AY208161 | TGAATCTTTTTGCCA |
| NO: 155 | Homo sapiens transforming growth | GCATTACATCCTGTG |
| | factor, beta 3 (TGFB3) gene, | GCTTTTTGAATTTGT |
| | complete cds | GGCCTTTTGAATCAC |
| SEQ ID | GI 209870107 NG_008768 | TGCTACTGTTGTAGA |
| NO: 156 | Homo sapiens propionyl-CoA | GAAGGGTAGGCCAGA |
| | carboxylase alpha subunit (PCCA), | GAATTTCTTTATGAG |
| | RefSeqGene on chromosome 13 | CAGCTCTTAGAAAGG |
| SEQ ID | GI 671744853 NG_034216 | AGGAATTCACACAGC |
| NO: 157 | Homo sapiens vascular endothelial | TAATCAATTCCATAA |
| | growth factor C (VEGFC), RefSeqGene | GGTGCAGTTGCTGGC |
| | on chromosome 4 | TTCTCTCTTCTTAAG |
| SEQ ID | GI 84043962 NM_015904 | CAGGGAAGACAAAAA |
| NO: 158 | Homo sapiens eukaryotic translation | TTCTAGATAAGCTCC |
| | initiation factor 5B (EIF5B), mRNA | GTCACACACATGTAC |
| | | AAGATGGTGAAGCAG |
| SEQ ID | GI 172072641 NM_005373 | TATCTGTCCCCTGCT |
| NO: 159 | Homo sapiens MPL proto-oncogene, | ACTAGATTGTGAGCT |
| | thrombopoietin receptor (MPL), mRNA | CCTTGAGGGAAAGGA |
| | | ACATGATTTATTTGT |
| SEQ ID | GI 15128210 AF402002 | GGTAAACTGGAATGA |
| NO: 160 | Homo sapiens interleukin 20 (IL20) | CCCACATCAATGGAA |
| | gene, complete cds | ACTAGTAAAGCGAGG |
| | | ATTTATTTGGACCCA |
| SEQ ID | GI 190570161 NG_008053 | CATGAGAGATCAGAG |
| NO: 161 | Homo sapiens acyl-CoA synthetase | GGCAGAAAGATAGAG |
| | long chain family member 4 (ACSL4), | AGTCTGCACACTTAT |
| | RefSeqGene on chromosome X | TACCTTTGCTCCTTT |
| SEQ ID | GI 343098512 NG_029658 | TAGAAGAAGAGCTGG |
| NO: 162 | Homo sapiens BTB domain and CNC | TAAAAAAGATAGAAC |
| | homolog 1 (BACH1), RefSeqGene on | CACAGACGATCCCCA |
| | chromosome 21 | GGTTCCTTTCTTGAA |
| SEQ ID | GI 1024846757 NG_047018 | GCTTAATGCTATGTC |
| NO: 163 | Homo sapiens histone deacetylase 1 | CAGAGATGATTTGCC |
| | (HDAC1), RefSeqGene on chromosome 1 | AGTGAAGCTAGATTT |
| | | GCCAGTGAAGAGAAT |
| SEQ ID | GI 341572559 NG_029522 | CAAAACCAGAATCTG |
| NO: 164 | Homo sapiens tachykinin receptor 1 | AACTGAGGGAGTCTG |
| | (TACR1), RefSeqGene on chromosome 2 | TCTTTGGAGCTCACT |
| | | CATTAGCTACTACAT |
| SEQ ID | GI 210032511 NG_008871 | CCTCTTACTTGTTCT |
| NO: 165 | Homo sapiens xanthine dehydrogenase | GTCTTTCTCTCTCTC |
| | (XDH), RefSeqGene on chromosome 2 | GCTTTAAAGGTGTCG |
| | | CTGCATTTTGGTAAC |
| SEQ ID | GI 430727969 NG_033033 | CTCTTTCCACTTAGC |
| NO: 166 | Homo sapiens ISG15 ubiquitin-like | ATAACGTTTTCAAGG |
| | modifier (ISG15), RefSeqGene on | TTCTTTCACATGAAT |
| | chromosome 1 | CAGTGCCTCAGTCCT |
| SEQ ID | GI 223890186 NG_009797 | GAAGGCAAGCACAGA |
| NO: 167 | Homo sapiens junctophilin 3 (JPH3), | TAAGTTGATCGTGGC |
| | RefSeqGene on chromosome 16 | TCAGTTCTTTGAAGG |
| | | AGATTAATAGGCTTT |
| SEQ ID | GI 1039673930 NG_050913 | CACAGATGGTGTTAT |
| NO: 168 | Homo sapiens nuclear receptor | TTCCTGTGTTAGACT |
| | subfamily 5 group A member 2 (NR5A2), | TCCTACTTACTGATA |
| | RefSeqGene on chromosome 1 | GCTGGGCTCACACTT |
| SEQ ID | GI 1073857156 NM_016930 | CAGATATTCATGAGG |
| NO: 169 | Homo sapiens syntaxin 18 (STX18), | ACCTGTTCAGAAGCA |
| | transcript variant 1, mRNA | ATTCAGCAACTACGA |
| | | ACAGAAGCTCACAAG |
| SEQ ID | GI 67515430 DQ088982 | GGGATTCTGCATCTC |
| NO: 170 | Homo sapiens glutathione peroxidase 6 | ATTTCACAGAGTGAC |
| | (olfactory) (GPX6) gene, complete cds | ATCCTCATGAGAACC |
| | | AAATCTTTGGAAGTT |
| SEQ ID | ref\|NG_021140.1\| | CTGAGCCACTGGATT |
| NO: 171 | Homo sapiens adiponectin, CIQ and collagen | TTCTCACCCTCAGAA |
| | domain containing | CTGTGAGCCAACAAA |
| | | TTTCTGTTCTTTATA |
| SEQ ID | GI 315506987 NG_028041 | GTTGCCAGGAGATTT |
| NO: 172 | Homo sapiens adenylate kinase 3 (AK3), | CACAGACTGGCAGTT |
| | RefSeqGene on chromosome 9 | ACTCATTTGTAATCA |
| | | ACAACCTTTTGCAAA |
| SEQ ID | GI 916259104 NG_042801 | TTAATCCCACTTTTA |
| NO: 173 | Homo sapiens bromodomain containing 2 | GGGTTGCCGAGGATT |
| | (BRD2), RefSeqGene on chromosome 6 | TTTCGAACGAGCAGA |
| | | AATGTATTGGTAACT |
| SEQ ID | GI 1140392578 NG_052831 | ATATTTATGGCTTTG |
| NO: 174 | Homo sapiens glutamate metabotropic | TTGAGTTTCCTTCCT |
| | receptor 5 (GRM5), RefSeqGene on | GTAGGGCTGTCTGTG |
| | chromosome 11 | TTCCTTTCACAAACT |
| SEQ ID | GI 261245113 NG_013061 | TTGACGTCATGAAAT |
| NO: 175 | Homo sapiens 3-hydroxymethyl-3-methylglutaryl- | TCAATGGCTCATCGC |
| | CoA lyase (HMGCL), RefSeqGene on chromosome 1 | CTTAGTCTCTAAGAA |
| | | AGCTTGTTGGCTAAA |
| SEQ ID | GI 90657247 DQ449938 | CTGCCAAATACAATT |
| NO: 176 | Homo sapiens Fas (TNFRSF6)-associated via | TTCGCAGTAGCCCTG |
| | death domain (FADD) gene, complete cds | TGGCTTAGATACTGT |
| | | TAGCTATCACTGTTC |
| SEQ ID | GI 440503012 JX512452 | TAATGATTACTGTCA |
| NO: 177 | Homo sapiens mitogen-activated protein | CGATGTGTGTGGTAC |
| | kinase 14 (MAPK14) gene, complete cds | AGTTTGACAAACCTG |
| | | GGTCCATATAGACCA |
| SEQ ID | GI 1025812271 NG_047045 | TGATTTATTCCATAT |
| NO: 178 | Homo sapiens activating transcription | CCCATCCCATTGTGT |
| | factor 2 (ATF2), RefSeqGene on chromosome 2 | CTCTGACCTCATCTG |
| | | CCACATTGTTTTGTC |
| SEQ ID | GI 821325145 NG_029706 | ATGGGCACTTGATCC |
| NO: 179 | Homo sapiens SH2B adaptor protein 1 | AATCAGCTACTCAGT |
| | (SH2B1), RefSeqGene on chromosome 16 | TCTGGAAATTTCATT |
| | | GGGAATCTCAGAAAA |
| SEQ ID | GI 254939589 NG_012569 | TAGTGGGTGGGAGAA |
| NO: 180 | Homo sapiens toll like receptor 7 (TLR7), | TTCCTTATTGCAATC |
| | RefSeqGene on chromosome X | ACACTCTACTTCTCC |
| | | ATCCAGAAAACTCTC |
| SEQ ID | GI 149274622 NM_032579 | AGAACTCAGTTTTGT |
| NO: 181 | Homo sapiens resistin like beta (RETNLB), | GACCATGACAGTAAT |
| | mRNA | GAAACCAGGGTCCCA |
| | | ACCAAGAAATCTAAC |
| SEQ ID | GI 238776831 NG_012261 | GAAGATAAGTCTCCA |
| NO: 182 | Homo sapiens aldehyde dehydrogenase 8 family | TTTGCCTTTTCGCAG |
| | member A1 (ALDH8A1), RefSeqGene on chromosome 6 | GGTTGCTTTTTGTTT |
| | | TTCTTTCTACCTGCT |
| SEQ ID | GI 38327038 NM_002154 | TATACTGATGCAGAA |
| NO: 183 | Homo sapiens heat shock protein family A | AGACGATCAGTGATG |
| | (Hsp70) member 4 (HSPA4), mRNA | GATGCAACACAGATT |
| | | GCTGGTCTTAATTGC |
| SEQ ID | GI 219555683 NM_176891 | TAGCCAACACAATAG |
| NO: 184 | Homo sapiens interferon epsilon (IFNE), mRNA | GGAAATGGAGAAAAT |
| | | GTACTCTGAACACCA |
| | | TGAAAAGGGAACCTG |
| SEQ ID | GI 633257761 NM_001143836 | ATGATGTTTCAAAGC |
| NO: 185 | Homo sapiens NADPH oxidase 4 (NOX4), | TGGTCTGCCATTCTA |
| | transcript variant NOX4B, mRNA | GATGAGCCTCCTTGC |
| | | TTATTTAAGTTCCAG |
| SEQ ID | GI 966209818 NM_014031 | CTGTTCCCTTACTTT |
| NO: 186 | Homo sapiens solute carrier family 27 member 6 | TGGGATGACTTCTGG |
| | (SLC27A6), transcript variant 1, mRNA | TTCGTGTTGAAGGTG |
| | | GTGCTCATTATAATT |
| SEQ ID | GI 152112977 EU012027 | TTTGTTTTGTTTGCT |
| NO: 187 | Homo sapiens high-mobility group box 1 | TGGCATAGCCCCTTC |
| | (HMGB1) gene, complete cds | AAGGAATTTAATCTC |
| | | TCGGCCATATTCTTG |
| SEQ ID | GI 342672042 NG_029626 | GGATTTTTAAGGGGT |
| NO: 188 | Homo sapiens synaptosome associated protein | AACCTGAGTTCTGTT |
| | 25 (SNAP25), RefSeqGene on chromosome 20 | TCTTTTCCCCCTTTT |
| | | CTAGGCTGATTCCAA |
| SEQ ID | GI 1024336756 NG_046908 | CTTTCTGACCTACTA |
| NO: 189 | Homo sapiens YY1 transcription factor (YY1), | CCTTTCCTCAAAGCT |
| | RefSeqGene on chromosome 14 | TTCAATCCTTTTGGG |
| | | CTTTCTCCACTTGAA |
| SEQ ID | GI 293629222 NG_017001 | ATTCTTTCAAGTCTG |
| NO: 190 | Homo sapiens hepatitis A virus cellular | AACAGGCCTCTTAGA |
| | receptor 1 (HAVCR1), RefSeqGene on | GTCTCTGCTCCTTGG |
| | chromosome 5 | AAGTGTAGAATTGAC |
| SEQ ID | GI 1024846756 NG_047011 | TTTTCCAGCATCTAG |
| NO: 191 | Homo sapiens mannose receptor C-type 1 | AGCTACATTCTATGA |
| | (MRC1), RefSeqGene on chromosome 10 | ACTCATCAGCTCATG |
| | | ACCCATTCCTCCTTC |
| SEQ ID | GI 908212430 NM_000412 | TATGATGTAGAAGCC |
| NO: 192 | Homo sapiens histidine rich glycoprotein | TTGGACTTGGAAAGC |
| | (HRG), mRNA | CCGAAAAACCTTGTC |
| | | ATAAACTGTGAAGTC |
| SEQ ID | GI 146045339 EF543149 | GCTCCAGTATTTATG |
| NO: 193 | Homo sapiens prostaglandin E synthase | TTGCCACTCTGTTGA |
| | (PTGES) gene, complete cds | ATGTACAGCCCATAA |
| | | CTTCCCTAACCAATT |
| SEQ ID | GI 308210781 NG_027799 | CTGTTGCAAGTCTTA |
| NO: 194 | Homo sapiens interferon related | AGTGGGGAATGAACT |
| | developmental regulator 1 (IFRD1), | TTGTGTTTTTAAGTT |
| | RefSeqGene on chromosome 7 | GGCAAAGACCAGTGT |
| SEQ ID | GI 345525590 NG_029936 | TTACATGCGCCATCT |
| NO: 195 | Homo sapiens mitogen-activated protein | CATCTAGTCTTCACA |
| | kinase 3 (MAPK3), RefSeqGene on | ACCACCCTCAAAAGA |
| | chromosome 16 | ACCATTATTTCATGG |
| SEQ ID | GI 256985185 NG_012797 | CHROMOSCTTTCCCT |
| NO: 196 | Homo sapiens membrane bound transcription | TAGCACTGCTAGATT |
| | factor peptidase, site 2 (MBTPS2), RefSeqGene on | GCCCTCCAAAAATGT |
| | chromoso [symbol] | CTTTTTGTAACTGAC |
| | | CCTCTTAC |
| SEQ ID | GI 254911084 NG_012516 | GTATAGGCCGGTCTA |
| NO: 197 | Homo sapiens moesin (MSN), RefSeqGene | AAGTGAGCTCTATGG |
| | on chromosome X | GCAGATCTACCCCTT |
| | | ACTTATTATTCCAGA |
| SEQ ID | GI 654825051 NG_029042 | TCAGTGCATAGAGAG |
| NO: 198 | Homo sapiens prolactin receptor (PRLR), | AAGGAGATAGTCAAA |
| | RefSeqGene on chromosome 5 | GAACCACTGTGGGGC |
| | | AGAAAAGGAAAATAT |
| SEQ ID | GI 189339276 NM_001204 | CCGGAAAAAGAAAGG |
| NO: 199 | Homo sapiens bone morphogenetic protein | CTAGAAAATACTCGC |
| | receptor type 2 (BMPR2), mRNA | ACTTCCTCAGAACCC |
| | | TCTTTCTTGTTAACG |
| SEQ ID | GI 378404896 NM_001256796 | ATTTATGATGCTAAA |
| NO: 200 | Homo sapiens purinergic receptor P2X 4 | ACAGATCCCTTCTGC |
| | (P2RX4), transcript variant 1, mRNA | CCCATATTCCGTCTT |
| | | GGCAAAATAGTGGAG |
| SEQ ID | GI 37577125 NM_194315 | AGGCTCCTAGGTTTA |
| NO: 201 | Homo sapiens ubiquitin conjugating enzyme | GCTTTTAAAAACCTG |
| | E2 J2 (UBE2J2), transcript variant 1, mRNA | AAAGGGGAAGCAAAA |
| | | ACCAAAATGTGTGAC |
| SEQ ID | GI 312434026 NM_002995 | AAATAATCCAGTCTT |
| NO: 202 | Homo sapiens X-C motif chemokine ligand 1 | CACTGGGTTCCCATT |
| | (XCL1), mRNA | CTGAGGGTCCACTAC |
| | | TCAAAAATTTGCTTC |
| SEQ ID | GI 325053739 NG_028167 | CCTGTAGTTCTAGTT |
| NO: 203 | Homo sapiens growth differentiation factor 3 | ACTTGGAGGCTGAGG |
| | (GDF3), RefSeqGene on chromosome 12 | CAGAAGATTCCCTTG |
| | | ATCCAGTAATTCAAG |
| SEQ ID | GI 169791020 NM_005328 | CAATGTGGTTTCACA |
| NO: 204 | Homo sapiens hyaluronan synthase 2 | AACATCACTTGTGGA |
| | (HAS2), mRNA | TGACCTACGAAGCGA |
| | | TTATCACTGGATTCT |
| SEQ ID | GI 3169541 AF043105 | GGTGATCTCATCTAG |
| NO: 205 | Homo sapiens glutathione S-transferase mu 3 | TCTCTGGTTTCAAAC |
| | (GSTM3) gene, complete cds | ACAGAGTGATCACTC |
| | | CTGTACTTGCTGTTT |
| SEQ ID | GI 324021724 NG_028154 | CTAAAGCTTCGCATA |
| NO: 206 | Homo sapiens fatty acid binding protein 5 | AATGATCTTTAGGGC |
| | (FABP5), RefSeqGene on chromosome 8 | AGACGTTAAAGGGCT |
| | | GGTACAACTACAACC |
| SEQ ID | GI 238814313 NG_012262 | TCTAGCTTACTTGTA |
| NO: 207 | Homo sapiens topoisomerase | CCAGTCACACTAGCC |
| | (DNA) 1 | TCCTTTGTTTCTTTT |
| | (TOP1), RefSeqGene on chromosome 20 | CCACACCAGATTCTT |
| SEQ ID | GI 260656026 NM_005518 | CGACCCTTCACCCTT |
| NO: 208 | Homo sapiens 3-hydroxy-3- | GACGATTTACAGTAC |
| | methylglutaryl-CoA synthase 2 | ATGATCTTTCATACA |
| | (HMGCS2), transcript variant 1, mRNA | CCCTTTTGCAAGATG |
| SEQ ID | GI 117606555 EF064752 | AACCAGGGGGAAGGT |
| NO: 209 | Homo sapiens CD5 molecule | GAGGGACTGTGGAAT |
| | (CD5) gene, complete cds | CTAACTTCCTTACTT |
| | | TACAGATTAAGAAAC |
| SEQ ID | GI 223972691 NG_009823 | AATCGCTCCTCTGGA |
| NO: 210 | Homo sapiens crystallin alpha A | ATTGTAACCTTGCAG |
| | (CRYAA), RefSeqGene on chromosome 21 | GGCAAAAGCTATCAT |
| | | TTCAAGATTTTTTGG |
| SEQ ID | GI 212549770 NG_008989 | GGATTCATGTTGCAG |
| NO: 211 | Homo sapiens matrix metallopeptidase 2 | ATATTGGTGATAGTG |
| | (MMP2), RefSeqGene on chromosome 16 | TGACATGAAGGCAGG |
| | | ATTGTGGTTAAGAGG |
| SEQ ID | GI 38202233 NM_052864 | GAATGAAGGCTGCAG |
| NO: 212 | Homo sapiens TRAP interacting protein | ATGGAAGTAAAATAG |
| | with forkhead associated domain | ATTATGCTGGATTAT |
| | (UFA), mRNA | CTGGGTGTGCCCAAT |
| SEQ ID | GI 148733545 EF636667 | ATCACACTGTGTTGC |
| NO: 213 | Homo sapiens caspase 4, apoptosis- | AACATCTGTACCTGA |
| | related cysteine peptidase | TGTTGCAAGACTAAC |
| | (CASP4) gene, complete cds | ATTTTTGCTACCTCT |
| SEQ ID | GI 54781374 AY795972 | ATGTTGCAGAATACT |
| NO: 214 | Homo sapiens matrix metalloproteinase 7 | CACTATTTCCAAATA |
| | (matrilysin, uterine) | GCCCAAAATGGACTT |
| | (MMP7) gene, complete cds | CCAAAGTGGTCACCT |
| SEQ ID | GI 915408 U30245 Human | AAAGCTTTACGGACA |
| NO: 215 | myelomonocytic specific protein | GTGATTCTGTCCTAA |
| | (MNDA) gene, 5′ flanking | ACAAGACAGTGACTC |
| | sequence and complete exon 1 | CAGGATTTCTGAAGA |
| SEQ ID | GI 335334987 NG_029076 | CTAGAAGCCTACAGC |
| NO: 216 | Homo sapiens C-X-C | CTTGGTTATTCCAGT |
| | motif chemokine receptor 3 | TCATCTTTTTAACAA |
| | (CXCR3), RefSeqGene on chromosome X | CCCTGCTGAGGAAAC |
| SEQ ID | GI 90577165 NM_032587 | AAGGAGGATTTGCTG |
| NO: 217 | Homo sapiens caspase recruitment | GCTGGAGTGGAGAAT |
| | domain family member 6 | TTGGAAATTCGAGAC |
| | (CARD6), mRNA | ATACAAACCATTAAT |
| SEQ ID | GI 217416377 NM_138394 | GATAATAATCTGGGA |
| NO: 218 | Homo sapiens heterogeneous nuclear | TCCATGGGTGGGCTT |
| | ribonucleoprotein L like | CAGAGAATCTGTGTA |
| | (HNRNPLL), transcript variant 1, mRNA | CCCCCTAAAATTGTA |
| SEQ ID | GI 187763976 EU518415 | ATCTGACTTTCACTT |
| NO: 219 | Homo sapiens serum/glucocorticoid | CTTGAAAGTGATCGG |
| | regulated kinase 1 isoform 2 | AAAGGGCAGTTTTGG |
| | (SGK1) mRNA, complete cds | AAAGGTTCTTCTAGC |
| SEQ ID | GI 1174251657 KY500414 | GGTAAGGACCTCCAA |
| NO: 220 | Homo sapiens LTB gene, complete cds | GACCTGAATAAGAGT |
| | | GTAAATAATCCGAAG |
| | | GTTCCAGTTCTGCTC |
| SEQ ID | GI 678246480 NG_034237 | CTTCACAAACCTACA |
| NO: 221 | Homo sapiens cytokine receptor like factor 2 | GGTTCTCGGAGCTCT |
| | (CRLF2), RefSeqGene on chromosome X and 7 | GGTGGAAGCTTCTCC |
| | | TATAAAAGTAATGAG |
| SEQ ID | GI 190358513 NG_008045 | TGTGTGCACATAAAT |
| NO: 222 | Homo sapiens dihydrolipoamide dehydrogenase | GCTCCTTATCTTGGT |
| | (DLD), RefSeqGene on chromosome 7 | CCCATGTCCTTCTTG |
| | | AGTTACCAATTTTGT |
| SEQ ID | GI 190358530 NG_008050 | CCATCTAAAGTCAGT |
| NO: 223 | Homo sapiens endothelin 3 | TATGGAGCTAGATGA |
| | (EDN3), RefSeqGene on chromosome 20 | GGGGCTGCCTGAATA |
| | | TTAAGGAAGAAGTGG |
| SEQ ID | GI 428673540 NM_006273 | GGACTTTATGAAGCA |
| NO: 224 | Homo sapiens C-C motif chemokine ligand 7 | CCTGGACAAGAAAAC |
| | (CCL7), mRNA | CCAAACTCCAAAGCT |
| | | TTGAACATTCATGAC |
| SEQ ID | GI 392055950 JQ867454 | CAATGTCAATAGCAG |
| NO: 225 | Homo sapiens ERBB receptor feedback inhibitor 1 | GAGTTGCTGCTCAGG |
| | (ERRFIl) gene, complete cds | AGATCAGAGTCCCAT |
| | | TAAAAACTGGATTTC |
| SEQ ID | GI 678246479 NG_034239 | TTGAGTGCTGAGCAG |
| NO: 226 | Homo sapiens lipopolysaccharide binding protein | GTAAATCCAAGAAGT |
| | (LBP), RefSeqGene on chromosome 20 | GATTTCCACAAGGTA |
| | | GAAAAAGTGGTGAAG |
| SEQ ID | GI 224809263 NG_011469 | GTTTATCTCCACAGT |
| NO: 227 | Homo sapiens microsomal | GCAGTTTTCTCAGTA |
| | triglyceride transfer protein | CCCATTCTTAGTTTG |
| | (MTTP), RefSeqGene on chromosome 4 | CATGCAGATGGACAA |
| SEQ ID | GI 974987499 NG_023044 | CACACTCTTCTTTGG |
| NO: 228 | Homo sapiens radixin | CATGTAACTTAGCAA |
| | (RDX), RefSeqGene on chromosome 11 | CTGGATCGATCAGTC |
| | | AAAGGGGAAAATGAA |
| SEQ ID | GI 344925862 NG_029825 | CACCTGCTCATCACA |
| NO: 229 | Homo sapiens ubiquitin specific peptidase 2 | TAAGTCAGAAGGGAC |
| | (USP2), RefSeqGene on chromosome 11 | AGATGTGGTCTTAAC |
| | | TCCTGAAATATCCAG |
| SEQ ID | GI 939106626 NG_029107 | CCTCTTTTATCTCTG |
| NO: 230 | Homo sapiens integrin subunit alpha 3 | CGTGTAAACCCCTAT |
| | (ITGA3), RefSeqGene on chromosome 17 | TTTGTTTTCAAGGTC |
| | | CCTGTCAGATGCTTC |
| SEQ ID | GI 41393574 NM_033214 | ACAAGCCAAAAACAC |
| NO: 231 | Homo sapiens glycerol kinase 2 | CTATGGAACAGGTTG |
| | (GK2), mRNA | CTTCTTACTGTGTAA |
| | | TACGGGTCGTAAATG |
| SEQ ID | GI 440503006 JX512449 | CCAGAAAACCTACTG |
| NO: 232 | Homo sapiens mitogen-activated protein kinase 1 | AAGACCTGAGGAATG |
| | (MAPK1) gene, complete cds | GAGGACTAGATTGAA |
| | | GAATTGGGAAGTCAC |
| SEQ ID | GI 326537295 NG_028240 | GTTTCATTCATCCTG |
| NO: 233 | Homo sapiens cell division cycle 6 | ACCACTGTATCCGTG |
| | (CDC6), RefSeqGene on chromosome 17 | TAATGTGATCTGAGC |
| | | TACAACAGGTGGTAT |
| SEQ ID | GI 397938 Z22576 H. sapiens CD69 gene | ATGTTTCTTCATGCT |
| NO: 234 | | CTGAGGACTGGGTTG |
| | | GCTACCAGAGGAAAT |
| | | GCTACTTTATTTCTA |
| SEQ ID | GI 210147537 NM_025106 | TTTAAAGATTTTCCT |
| NO: 235 | Homo sapiens splA/ryanodine receptor | CCCTCCTGTTTCAGG |
| | domain and SOCS box containing 1 | TGGGTCACATTCTGA |
| | (SPSB1), mRNA | TGAATGTTTCCCTTG |
| SEQ ID | GI 36030972 NM_181713 | GTCTGAGTTTTTAAA |
| NO: 236 | Homo sapiens UBX domain protein 2A | TAGCTAATACGACCG |
| | (UBXN2A), mRNA | GGTACAGTGGTTCAT |
| | | GCCTGTAATCCCAGA |
| SEQ ID | GI 3169541 AF043105 | TATGCTTTTGGGGCC |
| NO: 237 | Homo sapiens glutathione S-transferase mu 3 | TGGGAATTTGAGGTA |
| | (GSTM3) gene, complete cds | TTGGGACCAAGTCAT |
| | | TTCTTTAGATCTTTT |
| SEQ ID | GI 594591642 NG_034033 | GGTTAAATTAACTGT |
| NO: 238 | Homo sapiens phosphofructokinase, liver type | AGTCCTGATGGGACT |
| | (PFKL), RefSeqGene on chromosome 21 | GTCTCCATGTCTTTT |
| | | CCCTTCCCTTAACGT |
| SEQ ID | GI 270047500 NG_016132 | GAAATTCCCATGTAT |
| NO: 239 | Homo sapiens selectin L | TCTCCCAAGTATCTA |
| | (SELL), RefSeqGene on chromosome 1 | TTCACCAAGGCCACA |
| | | GCTCTAACCAAATGC |
| SEQ ID | GI 19923714 NM_138284 | TCAGAAGAATCCAAG |
| NO: 240 | Homo sapiens interleukin 17D | CAGGTATTGGCTTAG |
| | (IL17D), mRNA | TTGTAAGGGCTTTAG |
| | | GATCAGGCTGAATAT |
| SEQ ID | GI 31164 X51956 Human ENO2 | CCCGTGACCAATCTG |
| NO: 241 | gene for neuron specific | TTCTGTCTCAGATCC |
| | (gamma) enolase | TGATTAAAGTCATGG |
| | | GTTCTGAAAACTACT |
| SEQ ID | GI 184160977 NG_007553 | AAGAGTTAACAGTAG |
| NO: 242 | Homo sapiens actin, alpha, cardiac muscle 1 | TGCCCTGAGGTTAGT |
| | (ACTC1), RefSeqGene on chromosome 15 | TTCGGAGCACAATTA |
| | | TTGTTGGAGCTGATA |
| SEQ ID | GI 189339222 NG_007959 | GCCTGAAAACCAGCA |
| NO: 243 | Homo sapiens cytochrome P450 family 27 | CAGTATCATTTCCAC |
| | subfamily A member 1 | TGTATTCCACTCATC |
| | (CYP27A1), RefSeqGene on chromosome [symbol] | AAAGCAGTTACAGAG |
| SEQ ID | GI 628601932 NG_034128 | GGAAGCAAACTTCTA |
| NO: 244 | Homo sapiens guanylate cyclase 1 | GTACCCCTCCTTTCC |
| | soluble subunit alpha | TAAGCCTAGAAGCTT |
| | (GUCY1A3), RefSeqGene on chromosome 4 | ATGATGAGAAATGGG |
| SEQ ID | GI 176866166 NG_007524 | GAAACTTCAGTTGTA |
| NO: 245 | Homo sapiens KRAS proto-oncogene, GTPase | GCTGAGAGCAGGCCT |
| | (KRAS), RefSeqGene on chromosome 12 | GGAATCCCCATGCTT |
| | | ATACTTTTTATTTCC |
| SEQ ID | GI 193211405 NG_008118 | TAAGCACTTACGTGA |
| NO: 246 | Homo sapiens left-right determination factor 2 | GTAAATAATGCAGCT |
| | (LEFTY2), RefSeqGene on chromosome 1 | CAGATGCTGAGCTCT |
| | | AGTAGGAAATGCTGG |
| SEQ ID | GI 346644679 NG_029975 | TCAGAGATACAGGAA |
| NO: 247 | Homo sapiens transforming growth factor alpha | CACCAGTGAGTGTGT |
| | (TGFA), RefSeqGene on chromosome 2 | TTGCTTCAATCATCT |
| | | GGATTGTAAACTCCT |
| SEQ ID | GI 206597534 NM_001639 | CTATGAAATCAGAGG |
| NO: 248 | Homo sapiens amyloid P component, serum | ATATGTCATCATCAA |
| | (APCS), mRNA | ACCCTTGGTGTGGGT |
| | | CTGAGGTCTTGACTC |
| SEQ ID | GI 224809272 NG_011478 | AGTGTGTGAATATCA |
| NO: 249 | Homo sapiens uncoupling protein 2 | GGAAGATGACTGAAC |
| | (UCP2), RefSeqGene on chromosome 11 | GTCTTTGGGACTCCG |
| | | TTTCCTCATTGTAAA |
| SEQ ID | GI 167860125 NM_002639 | TTCTTTGGGATAGAG |
| NO: 250 | Homo sapiens serpin family B member 5 | AATGTTCCAGACATT |
| | (SERPINB5), mRNA | CTCGCTTCCCTGAAA |
| | | GACTGAAGAAAGTGT |
| SEQ ID | GI 429535873 NG_033024 | ATTGTGAAAATCAAA |
| NO: 251 | Homo sapiens colony stimulating factor 2 | CACTTGTCAGCCCCT |
| | (CSF2), RefSeqGene on chromosome 5 | CAAGAGCCTTTAGAT |
| | | TTCCTACTTCCACAC |
| SEQ ID | GI 563317866 NG_033843 | TTTTCCTTATAAGGA |
| NO: 252 | Homo sapiens DnaJ heat shock protein family | CACTAATCCCATGAT |
| | (Hsp40) member C6 | GGGGGCCCTACCCAC |
| | (DNAJC6), RefSeqGene on chromosome [symbol] | ATAACCTCATTTAAA |
| SEQ ID | GI 354725887 NG_030444 | GGGTAAAAAGCCTTA |
| NO: 253 | Homo sapiens hepatitis A virus cellular receptor 2 | AGTTCCCTTAGCTTC |
| | (HAVCR2), RefSeqGene on chromosome 5 | AGATTCCTGGGCTGT |
| | | AACATGTTGATGATG |
| SEQ ID | GI 359751452 NG_031884 | GTTAATGGGGAATGG |
| NO: 254 | Homo sapiens mitogen-activated | ATTACCATGAGGAAA |
| | protein kinase kinase kinase 1 | AATATGGGGCATTCA |
| | (MAP3K1), RefSeqGene on chromosome 5 [symbol] | GAAATGCTCTCAGGT |
| SEQ ID | GI 122937306 NM_001080454 | GCTGACTTTGAAGCC |
| NO: 255 | Homo sapiens acyl-CoA synthetase | ATAAATCGCTGTAAC |
| | medium chain family member 4 | AGGCCATTGCCTAAA |
| | (ACSM4), mRNA | AACTTTAACTTTGCT |
| SEQ ID | GI 260656026 NM_005518 | AACAGTGCTCATGGA |
| NO: 256 | Homo sapiens 3-hydroxy-3- | ACTCTTCCAGGATTC |
| | methylglutaryl-CoA synthase 2 | AGGCAATACTGATAT |
| | (HMGCS2), transcript variant 1, mRNA | TGAGGGCATAGATAC |
| SEQ ID | GI 527317415 NM_000304 | AGAAAGCAGACAAAG |
| NO: 257 | Homo sapiens peripheral myelin protein 22 | AAAAAAGAGCTAGCC |
| | (PMP22), transcript variant 1, mRNA | CAAAATCCCAAACTC |
| | | AAACCAAACCAAACA |
| SEQ ID | GI 56788327 AY851164 | AATCATCCAACTTCT |
| NO: 258 | Homo sapiens carboxylesterase 2 | ATGGGCCTCAGTTTC |
| | (intestine, liver) | AACACCCATAAAATA |
| | (CES2) gene, complete cds | GGCACAACAATCTCC |
| SEQ ID | GI 299758395 NG_011675 | ATAAAAAGCTGAGAT |
| NO: 259 | Homo sapiens glial cell | CTGGGGACAATGTGA |
| | derived neurotrophic factor | GAATCCTTAGCAGTA |
| | (GDNF), RefSeqGene on chromosome 5 | TGTTGCTGTTGTTGC |
| SEQ ID | GI 518828582 NM_001278579 | ATTACAACATCCTGC |
| NO: 260 | Homo sapiens activin A receptor type 2A | TCTATTCCTTGGTGC |
| | (ACVR2A), transcript variant 1, mRNA | CACTTATGTTAATTG |
| | | CGGGGATTGTCATTT |
| SEQ ID | GI 150010588 NM_003641 | CCTGCCCCTAGATAC |
| NO: 261 | Homo sapiens interferon induced | AGCAGTTTATACCCA |
| | transmembrane protein 1 | CACACCTGTCTACAG |
| | (IFITM1), mRNA | TGTCATTCAATAAAG |
| SEQ ID | GI 960513892 NM_001317781 | TTTGTGTTGCACGCA |
| NO: 262 | Homo sapiens sorting nexin 25 | TGCTTGAGGAACTCA |
| | (SNX25), mRNA | GATGATGAAGTAAGA |
| | | TTTCTACAAACGTGT |
| SEQ ID | GI 1049010852 AH002976 | CAGCTTTGTGTCAAT |
| NO: 263 | Homo sapiens S protein | TACCCTGGAGGTTAC |
| | (PROS1) gene, complete cds; and | ACTTGCTATTGTGAT |
| | PS-alpha gene, complete sequence | GGGAAGAAAGGATTC |
| SEQ ID | GI 194018739 NG_008192 | GCTAATCTTTCCTCC |
| NO: 264 | Homo sapiens CD36 molecule | AGGAAAGGGTTGTGA |
| | (CD36), RefSeqGene on chromosome 7 | TTTCTCCACTTTTCA |
| | | CTTCATGAACAACCA |
| SEQ ID | GI 257467496 NG_012839 | GTTGAAGTTGAGGTG |
| NO: 265 | Homo sapiens glutamate | AGAGATCTGACAGAA |
| | metabotropic receptor 1 | GAGCTGCCTCTTGCT |
| | (GRM1), RefSeqGene on chromosome 6 | AGATAGTATTTGTGC |
| SEQ ID | GI 327180724 NG_028256 | CCCCATTTTGGTGTT |
| NO: 266 | Homo sapiens mitogen- | GATATGGAAATTCCT |
| | activated protein kinase kinase 3 | AGGTCACTATGCAGA |
| | (MAP2K3), RefSeqGene on chromosome 17 | CAAGAAAACCAGGAC |
| SEQ ID | GI 1024846748 NG_046980 | TTTTGGACACCTTAG |
| NO: 267 | Homo sapiens synaptonemal complex protein 2 | CTATGTTAGTTACCA |
| | (SYCP2), RefSeqGene on chromosome 20 | GGGCAGAGGGGACTT |
| | | TGAATTATCTACTGC |
| SEQ ID | GI 1159261282 NG_052987 | CTTTGGACAGAACTA |
| NO: 268 | Homo sapiens zinc finger protein 148 | GCCAAAATGATGCCT |
| | (ZNF148), RefSeqGene on chromosome 3 | ATTTGAATAGCCCGA |
| | | GCCTTAACTTTGTGA |
| SEQ ID | GI 6272556 AF123887 | GAACTAAGGGCTTTA |
| NO: 269 | Homo sapiens EROIL | TCCAAAGTGTTACCA |
| | (EROIL) mRNA, | TTCTTCGAGCGCCCA |
| | | GATTTTCAACTCTTT |
| SEQ ID | GI 41349444 NM_014011 | TCTCATGTCTTTTCG |
| NO: 270 | Homo sapiens suppressor of cytokine signaling 5 | TTAATGTGCTCTGTA |
| | (SOCS5), transcript variant 1, mRNA | CCACTGGTGAGTGCT |
| | | CCATAGTTTCCTTAC |
| SEQ ID | GI 257467496 NG_012839 | AAGTCAGAGCTGGGA |
| NO: 271 | Homo sapiens glutamate metabotropic receptor 1 | TGAGGTGAAAGTGTT |
| | (GRM1), RefSeqGene on chromosome 6 | ATAGGAAGAAGAAAT |
| | | AGCATGTGCATAGGG |
| SEQ ID | GI 195234781 NG_008319 | GGTTGCTGTTTACTT |
| NO: 272 | Homo sapiens pyruvate carboxylase | TCCTCTTCCAGGAAC |
| | (PC), RefSeqGene on chromosome 11 | TGAAGCTGTTTCCCA |
| | | TTGGATTTCTTTACT |
| SEQ ID | GI 117606467 EF064708 | CTGAGGACATTATCA |
| NO: 273 | Homo sapiens tumor necrosis factor | AAAGGAGAGCAAGAA |
| | (ligand) family, member 10 | AGAGAAGAGAGAAAT |
| | (TNFSF10) gene, complete cds | GGGCTTGAGGTGAGT |
| SEQ ID | GI 208022628 NM_004887 | GAACTCAAGTTTGGT |
| NO: 274 | Homo sapiens C-X-C motif chemokine ligand 14 | TGCCAGAAAAATGTG |
| | (CXCL14), mRNA | CTTCATTCCCCCCTG |
| | | GTTAATTTTTACACA |
| SEQ ID | GI 387763637 NM_001098272 | CCCTTCTCTTGCATT |
| NO: 275 | Homo sapiens 3-hydroxy-3- | CCTCAAACATGCTGA |
| | methylglutaryl-CoA synthase 1 | GTTTATTCCAGCCTT |
| | (HMGCS1), transcript variant 1, mRNA | AAGCCTTTGATCTAC |
| SEQ ID | GI 71648775 DQ143945 | CAGGCATGTATGAAT |
| NO: 276 | Homo sapiens adrenomedullin | GAAAGAGTGACTCAA |
| | (ADM) gene, complete cds | GATTCCTGTCATGGA |
| | | ATTTCTGTGCCAGTG |
| SEQ ID | GI 117606501 EF064725 | TTTCCATGAGCTACA |
| NO: 277 | Homo sapiens interferon, beta 1, fibroblast | ACTTGCTTGGATTCC |
| | (IFNB1) gene, complete cds | TACAAAGAAGCAGCA |
| | | ATTTTCAGTGTCAGA |
| SEQ ID | GI 298104098 NG_021428 | CTTCAGGACAAAGAA |
| NO: 278 | Homo sapiens C-C motif chemokine receptor 2 | GGAGCCTAGAGACAG |
| | (CCR2), RefSeqGene on chromosome 3 | AAATGACAGATCTCT |
| | | GCTTTGGAAATCACA |
| SEQ ID | GI 345478675 NG_029883 | GCCTTAGAGACAAAA |
| NO: 279 | Homo sapiens death associated protein kinase 1 | AGAGCTCAAACTCAA |
| | (DAPK1), RefSeqGene on chromosome 9 | ACAGTCCAAGCATAG |
| | | CACAGATCCAATAGC |
| SEQ ID | GI 41872630 NM_004104 | CCCCGTTTCATTTTT |
| NO: 280 | Homo sapiens fatty acid synthase | ACACCAATTTGGTAA |
| | (FASN), mRNA | AAATGCTGCTCTCAG |
| | | CCTCCCACAATTAAA |
| SEQ ID | GI 698320876 NM_015995 | GCCACGTTTCATCTT |
| NO: 281 | Homo sapiens Kruppel like factor 13 | TCCTGGATCACTACA |
| | (KLF13), transcript variant 1, mRNA | GTGAAGTATTACAGT |
| | | TGTACAGTTCCCAGT |
| SEQ ID | GI 1212624021 NM_017523 | TAACCAAAACTGCAT |
| NO: 282 | Homo sapiens XIAP associated factor 1 | GATACTGGTACAAAA |
| | (XAF1), transcript variant 1, mRNA | GCATGGTGCTGGTAC |
| | | AAAAGCAGACACATA |
| SEQ ID | GI 68248543 DQ096732 | GGGACCAATGATTAC |
| NO: 283 | Homo sapiens glutathione peroxidase 7 | CTCATAGGGCTGTTG |
| | (GPX7) gene, complete cds | TGAGGATTAGGATGA |
| | | AATACCTGTGAAAGT |
| SEQ ID | GI 228008299 NG_011964 | TAAATGGAGTTGGAC |
| NO: 284 | Homo sapiens arginase 2 | AATGTGGACAGAATC |
| | (ARG2), RefSeqGene on chromosome 14 | CACTACCCAGGTTGG |
| | | GAATTAATCTGATGA |
| SEQ ID | GI 1122782504 NG_013302 | TGTCTGATGGAAATA |
| NO: 285 | Homo sapiens catenin beta 1 | GATGTTATTTGTGTA |
| | (CTNNB1), RefSeqGene on chromosome 3 | GTCTCATGGGTGGCC |
| | | TGGCACTGAGTAATT |
| SEQ ID | GI 209447065 NG_008724 | AAAAGAGCCTCAGAT |
| NO: 286 | Homo sapiens NLR family | GAGGTTACAGTTCTC |
| | apoptosis inhibitory protein | TTGAGACCAAGGTGT |
| | (NAIP), RefSeqGene on chromosome 5 | CTTGCATGTATCATC |
| SEQ ID | GI 353249887 NG_030361 | ATCTGTATCATCAGT |
| NO: 287 | Homo sapiens Spl transcription factor | GCCTCTTTGAAGGTG |
| | (SP1), RefSeqGene on chromosome 12 | GGAAACATTAGTGAA |
| | | AATTCTGTTGGTGCC |
| SEQ ID | GI 222080101 NM_022121 | CTGGATACAGTGTGC |
| NO: 288 | Homo sapiens PERP, TP53 apoptosis effector | AGTGCATTCTTCCTG |
| | (PERP), mRNA | AATTTTGCAATTCTG |
| | | GTATCTGGGTGTATT |
| SEQ ID | GI 371875636 NM_182919 | CAGAACTGGATACGG |
| NO: 289 | Homo sapiens toll like receptor | TGTTTACAATTCCAA |
| | adaptor molecule 1 | TCTCTCTATTTCTGG |
| | (TICAM1), mRNA | GTGAAGGGTCTTGGT |
| SEQ ID | GI 226061859 NG_011749 | AGTTCATTCTCACTC |
| NO: 290 | Homo sapiens peroxisome proliferator | TTCTCTCTTGGTTGA |
| | activated receptor gamma | GTCTTGCCTTTCTTC |
| | (PPARG), RefSeqGene on chromosome 3 | ACTCTGTTCCCTTTC |
| SEQ ID | GI 281332104 NG_016228 | CTCAGTAACTTGGTT |
| NO: 291 | Homo sapiens toll like receptor 1 | TTGCATTGACTTTCC |
| | (TLR1), RefSeqGene on chromosome 4 | ATTCTTCCTTGGTCA |
| | | CTCTTCTCATTCGGG |
| SEQ ID | GI 32815079 AY338491 | CTGCAGGCCAAAATG |
| NO: 292 | Homo sapiens cyclin B1 | CCTATGAAGAAGGTA |
| | (CCNB1) gene, complete cds | ACTCTCTTCCTGACC |
| | | TAACTTCTGTAAGAG |
| SEQ ID | GI 326806980 NG_028248 | AAGGGATGGCCAAAT |
| NO: 293 | Homo sapiens aryl hydrocarbon | ACCTGCAGAGATCAT |
| | receptor nuclear translocator | CACATTTTTACCTGT |
| | (ARNT), RefSeqGene on chromosome 1 | CTTACTGTAGTCGTT |
| SEQ ID | GI 939106626 NG_029107 | TGAGATCTCTAAGCC |
| NO: 294 | Homo sapiens integrin subunit alpha 3 | ACTCTCAGATCCACA |
| | (ITGA3), RefSeqGene on chromosome 17 | CTTTACAGCTTTCCA |
| | | AAAAGTGTTCCTGAC |
| SEQ ID | GI 340007370 NG_U29431 | TCAAGCTTCAGCCTA |
| NO: 295 | Homo sapiens ras-related C3 | CTAAATCTCTGTTTT |
| | botulinum toxin substrate 1 | TATGTTTTGTTTTCC |
| | (rho family, small G1P binding | AGGCCCTATGCCTGG |
| | protein Rac1) | |
| | (RAC [symbol] | |
| SEQ ID | GI 1027860277 NG_047185 | ACTCAGAAAAATAGC |
| NO: 296 | Homo sapiens NLR family member XI | TTGTGGGAGCCCAGT |
| | (NLRX1), RefSeqGene on chromosome 11 | GTTTGGACTCTATAA |
| | | GTAACCTTGGACTTT |
| SEQ ID | GI 1024336768 NG_046906 | GTTTTCTGCACTCAT |
| NO: 297 | Homo sapiens TANK binding kinase 1 | GTCGCTATGTATGTG |
| | (TBK1), RefSeqGene on chromosome 12 | CCCTTGGACTGTAAT |
| | | GCTTTTGACAGTTTA |
| SEQ ID | GI 209862906 NM_006475 | CGTGAGGAAGTTGCA |
| NO: 298 | Homo sapiens periostin | AGCCAACAAAAAAGT |
| | (POSTN), transcript variant 1, mRNA | TCAAGGATCTAGAAG |
| | | ACGATTAAGGGAAGG |
| SEQ ID | GI 293629222 NG_017001 | GGCTCTTGTTTCTTG |
| NO: 299 | Homo sapiens hepatitis A virus | TTTGACTTATGCTCA |
| | cellular receptor 1 | CTCTCATGTTGATTT |
| | (HAVCR1), RefSeqGene on chromosome 5 | CTGACTCCAGCCAAG |
| SEQ ID | GI 193290114 NG_008154 | TAGATGGGTTGCTCA |
| NO: 300 | Homo sapiens insulin receptor substrate 2 | TTAGTGCATATCACT |
| | (IRS2), RefSeqGene on chromosome 13 | GTGAGTTGACCAGGG |
| | | GTAATAGCATTTGAG |
| SEQ ID | GI 584593316 NG_033952 | ACAGTCTACTCACTT |
| NO: 301 | Homo sapiens mitogen-activated | GGTTGTTTTTTTCTG |
| | protein kinase kinase 4 | GTGGCCAGCCCCTAT |
| | (MAP2K4), RefSeqGene on chromosome 17 | CCTGAAGTTATATAG |
| SEQ ID | GI 300244554 NG_023330 | ATTGAAAACAAGAGA |
| NO: 302 | Homo sapiens SMAD family member 7 | CCAGCAAGGGTCCTT |
| | (SMAD7), RefSeqGene on chromosome 18 | GTGAATTATGATGGT |
| | | TTGGCGTGAGAATAG |
| SEQ ID | GI 32891798 AY341246 | GGGAGATCTCAAGTA |
| NO: 303 | Homo sapiens catechol-O-methyltransferase | TCCCAAAGGAGCCAC |
| | (COMT) gene, complete cds | TGATGTTCCAAATTA |
| | | TCCTTGGTAAAGTCA |
| SEQ ID | GI 22128580 AF533019 | AACTTCCTGGATTAT |
| NO: 304 | Homo sapiens growth arrest and | CCTCGCCAAGGACTT |
| | DNA-damage-inducible, beta | TGCAATATATTTTTC |
| | (GADD45B) gene, complete cds | CGCCTTTTCTGGAAG |
| SEQ ID | GI 37654535 AY434089 | CATTTCTCAGTTAAA |
| NO: 305 | Homo sapiens insulin-like growth | CTTTGTCAAACTGTG |
| | factor binding protein 1 | AACACATGGACTTTC |
| | (IGFBP1) gene, complete cds | AGGAGAGGAGCCCAC |
| SEQ ID | GI 395393980 NG_011966 | CTTCCACAACATCTG |
| NO: 306 | Homo sapiens mitogen-activated | TGCCCATCTACTCTG |
| | protein kinase kinase kinase 7 | CTTTCTTCTGTTACT |
| | (MAP3K7), RefSeqGene on chromosome 6 | CTGGCTAAACTATCC |
| SEQ ID | GI 209870110 NM_013943 | AAAAGCTAGCTAGTA |
| NO: 307 | Homo sapiens chloride intracellular channel 4 | AAAAGGACGACCCAG |
| | (CLIC4), mRNA | CAACATGCTTTAACC |
| | | CCATTGTATGTTTGT |
| SEQ ID | GI 148227403 NM_005292 | AAGCATTGCTTTATG |
| NO: 308 | Homo sapiens G protein-coupled receptor 18 | GCTTCTTGCCTTTAT |
| | (GPR18), transcript variant 1, mRNA | TAGTGCTGACAGATA |
| | | CATGGCCATTGTACA |
| SEQ ID | GI 315630394 NM_002611 | CTTCTGTGTATATAG |
| NO: 309 | Homo sapiens pyruvate dehydrogenase kinase 2 | TTAGTTTTATAACCC |
| | (PDK2), transcript variant 1, mRNA | TGAATGCCCCCACCC |
| | | TTCCCCTAAGCACAC |
| SEQ ID | GI 38202233 NM_052864 | ACCAGTTTTGAAGAT |
| NO: 310 | Homo sapiens TRAF interacting protein | GCTGACACAGAAGAG |
| | with forkhead associated domain | ACAGTAACTTGTCTC |
| | (TIFA), mRNA | CAGATGACGGTTTAC |
| SEQ ID | GI 117606531 EF064740 | GTAGATGCTTAAACC |
| NO: 311 | Homo sapiens Epstein-Barr virus induced gene 3 | TCAGCTTCCCTATCT |
| | (EBI3) gene, complete cds | GGAAAATGGGCTAGT |
| | | AGTCCTCACCTAGAT |
| SEQ ID | GI 193290117 NG_008157 | GAGGCTGATGAAGTG |
| NO: 312 | Homo sapiens hydroxysteroid | AATTGGGAGACAAAG |
| | 17-beta dehydrogenase 3 | GTGCTTAATTCTTTA |
| | (HSD17B3), RefSeqGene on chromosome 9 | GCCATCCTGTTGATG |
| SEQ ID | GI 237858771 NG_012125 | CTGTCAAGGTACTGT |
| NO: 313 | Homo sapiens selectin P | AACAGTGGTAATGTT |
| | (SELP), RefSeqGene on chromosome 1 | TTTCTGTGTGTCTCA |
| | | GCTCGTTAGAGGCAT |
| SEQ ID | GI 194294528 NG_008241 | TGCAGAGTGGGTTGA |
| NO: 314 | Homo sapiens succinate-CoA ligase | TACAATTTGACTGAA |
| | ADP-forming beta subunit | AGCCCCAAACCTCTA |
| | (SUCLA2), RefSeqGene on chromosome 13 | ATCACATTGTCAATC |
| SEQ ID | GI 94717603 DQ508420 | GTGATAAACCACATG |
| NO: 315 | Homo sapiens caspase 5, apoptosis- | CTAAAGAACAACGTG |
| | related cysteine peptidase | GCTGGACAAACATCT |
| | (CASP5) gene, complete cds | ATCCAGACCCTAGTA |
| SEQ ID | GI 47777672 AY623118 | CAATGACTTTGTTTT |
| NO: 316 | Homo sapiens hexokinase 2 | AAGGGGAAACTCTTG |
| | (HK2) gene, complete cds | AAGGAATGGGGCCTA |
| | | CTTGTTGCTATACCC |
| SEQ ID | GI 74418831 DQ185447 | GAAGTGCTCATGTGT |
| NO: 317 | Homo sapiens interferon, alpha l | GGAGAGAGTGTAGAC |
| | (IFNA1) gene, complete cds | AGAGAAAAGGACTTA |
| | | AGCAGGATTTTGAGG |
| SEQ ID | GI 219521814 NG_009215 | CCATACAGCTTGTTT |
| NO: 318 | Homo sapiens bone morphogenetic protein 4 | CTGTTGTTTGGTGCA |
| | (BMP4), RefSeqGene on chromosome 14 | TTAGGCCAAAAGACC |
| | | TAGGCCTAATAGATG |
| SEQ ID | GI 834400732 NG_042181 | GAAAAGTAAGGGACT |
| NO: 319 | Homo sapiens collagen type XII alpha 1 chain | GGAAGGGACTGTGCT |
| | (COL12A1), RefSeqGene on chromosome 6 | CTTCCATTCTCAAAT |
| | | TCTAATCTGTGTCCA |
| SEQ ID | GI 262331531 NG_013343 | CAGGGGATGGAGGTA |
| NO: 320 | Homo sapiens endothelin receptor type A | AAAAAAAAAAAAAAG |
| | (EDNRA), RefSeqGene on chromosome 4 | CAGAGTTGGGTAGTG |
| | | GAGACCCATCCTTAG |
| SEQ ID | GI 1020158924 NM_002895 | CATTCAGAGTTGTCT |
| NO: 321 | Homo sapiens RB transcriptional corepressor like 1 | CACCCCTTCTCGTGC |
| | (RBL1), transcript variant 1, mRNA | CTTTTCTCTCCTTGA |
| | | GCTTTATAAACAGAT |
| SEQ ID | GI 187423911 NM_003764 | GTTCTTTCCTAATTC |
| NO: 322 | Homo sapiens syntaxin 11 | TTCCCAAGCTGTGAG |
| | (STX11), mRNA | TCAGAAAGTCCTGGA |
| | | AGGAGTTGTAGGAAG |
| SEQ ID | GI 226958454 NG_011902 | CTTCGAAATTAAATG |
| NO: 323 | Homo sapiens CCHC-type zinc finger | CGGTTAGCATCATAT |
| | nucleic acid binding protein | AGTTCATTTTCCCCC |
| | (CNBP), RefSeqGene on chromosome 3 | TCCATGCTGCTGTGT |
| SEQ ID | GI 207440658 NG_008647 | TCCTTTTCACTTAGT |
| NO: 324 | Homo sapiens GTP cyclohydrolase 1 | GGAAGAGCAGTTGCA |
| | (GCH1), RefSeqGene on chromosome 14 | GACATGAAAGGTTTT |
| | | CAAGAAGCCTTTCTG |
| SEQ ID | GI 1028908925 NG_050621 | CTGGCAACCCTGAAA |
| NO: 325 | Homo sapiens glutathione peroxidase 4 | TAAACTCCCATTTCA |
| | (GPX4), RefSeqGene on chromosome 19 | GTGCACATGTTTGCA |
| | | ACAAACTTAAGGAAA |
| SEQ ID | GI 193290117 NG_008157 | CTTGTAGTTGTGAGT |
| NO: 326 | Homo sapiens hydroxysteroid 17-beta dehydrogenase 3 | ATGATCAGGGTGTAC |
| | (HSD17B3), RefSeqGene on chromosome 9 | TATAGGGTGAGAGGA |
| | | GTCCTAAGCAAGTGC |
| SEQ ID | GI 238018080 NG_012203 | CGCAGATTTGCCTTC |
| NO: 327 | Homo sapiens phospholipase A2 group IVA | CTGGATCTAAATGCA |
| | (PLA2G4A), RefSeqGene on chromosome 1 | CTGTGGAGTCAAAGA |
| | | TTAAGGTGTTTCTTT |
| SEQ ID | GI 1028916196 KU928257 | AATCTCTAACTTCCA |
| NO: 328 | Homo sapiens complement decay-accelerating factor | AGGTCCCACCAACAG |
| | (CD55) gene, CD55-203A allele, complete cds | TTCAGAAACCTACCA |
| | | CAGTAAATGTTCCAA |
| SEQ ID | GI 93009032 DQ464902 | AAGCTGAAGAGGAAG |
| NO: 329 | Homo sapiens nexilin | CAAGAAAACGTTTAG |
| | (NEXN) mRNA, complete cds | AAGAAGAGAAGCGTG |
| | | CTTTTGAAGAAGCAA |
| SEQ ID | GI 284172478 NM_052946 | TTCCAAATTCTAGGA |
| NO: 330 | Homo sapiens nitric oxide synthase trafficking | GACCCTAGAGATGAT |
| | (NOSTRIN), transcript variant 1, mRNA | CCAGTATAACCCCTG |
| | | GTGTCACAGAAACAG |
| SEQ ID | GI 356582493 NM_001244 | CGGGATACTTCTTTT |
| NO: 331 | Homo sapiens TNF superfamily member 8 | ATCTGCAGATGGCCT |
| | (TNFSF8), transcript variant 1, mRNA | AATACTTAGTTGTCC |
| | | AAGTCGCGATCAAGG |
| SEQ ID | GI 20147702 AF493905 | ACCCACTTCACATGT |
| NO: 332 | Homo sapiens guanine nucleotide | GCCACAGATACTAAG |
| | binding protein alpha il | AATGTGCAGTTTGTT |
| | (GNAIl) mRNA, complete cds | TTTGATGCTGTAACA |
| SEQ ID | GI 156071494 NM_002787 | ATCTGCAATGAAGCT |
| NO: 333 | Homo sapiens proteasome subunit alpha 2 | GGATTTAGGAGGCTT |
| | (PSMA2), mRNA | ACTCCAACTGAAGTT |
| | | AAGGATTACTTGGCT |
| SEQ ID | GI 41353192 AY486465 | GAGGGATATAGTATT |
| NO: 334 | Homo sapiens glutathione transferase kappa 1 | CACAAGTCTCCACAA |
| | (GSTK1) gene, complete cds | ACTCCAACACAAACC |
| | | TCACCAACACATCCA |
| SEQ ID | GI 359718928 NG_031876 | GAAACTTGCAAGGCT |
| NO: 335 | Homo sapiens biliverdin reductase A | GAAGAAAGAGAGGCA |
| | (BLVRA), RefSeqGene on chromosome 7 | GTTAGTCATAGGAGA |
| | | CAGTTCAGTCAGATG |
| SEQ ID | GI 345110544 NG_029851 | CTTATATCACTGGGA |
| NO: 336 | Homo sapiens phospholipase D1 | AACTAGAAGCAGTTT |
| | (PLD1), RefSeqGene on chromosome 3 | CCCAGCTCCCATATT |
| | | CTCTTGCATCCATCC |
| SEQ ID | GI 336088682 NG_029094 | CGGTTGGATCAGGAT |
| NO: 337 | Homo sapiens apoptotic | GAGAGTTTTTCCCAG |
| | peptidase activating factor 1 | AGGCTTCCACTTAAT |
| | (APAF1), RefSeqGene on chromosome 12 | ATTGAAGAGGCTAAA |
| SEQ ID | GI 746815960 NM_004929 | TGAAATTTACAGTTA |
| NO: 338 | Homo sapiens calbindin 1 | CTGCCTAGGAGTTAA |
| | (CALB1), mRNA | TGATCGTTCTGGGTC |
| | | AGCTTGAATGTCCCC |
| SEQ ID | GI 52352731 AY751746 | AGACACAGCCTGGAC |
| NO: 339 | Homo sapiens jun B proto-oncogene | CACTCCAGACAAGAC |
| | (JUNB) gene, complete cds | AATAAATAAACAAGC |
| | | AAACATGGTTCTTTG |
| SEQ ID | GI 336391122 NG_029141 | ATCTTTTCAGAATTG |
| NO: 340 | Homo sapiens interferon gamma | GTCTCCTGGAGGGGG |
| | inducible protein 16 | CATAATTGGGTAATT |
| | (IFI16), RefSeqGene on chromosome 1 | TTTCTGCCAAATGAG |
| SEQ ID | GI 336285236 NG_029146 | ACTTCTGTTTGTCTT |
| NO: 341 | Homo sapiens MCL1, BCL2 | ACGCTTCTCTCAGGG |
| | family apoptosis regulator | AAAAACATGCAGTCC |
| | (MCL1), RefSeqGene on chromosome 1 | TCTAGTGTTTCATGT |
| SEQ ID | GI 105990527 NM_012258 | GCTTTTTAAAGAACT |
| NO: 342 | Homo sapiens hes related family Bhlh | GATGTAGAATGAGGG |
| | transcription factor with YRPW motif 1 | AGGGGAAAGTTTAAA |
| | (HEY1), transcript variant 1, | ATCCCAGCTGGGCTG |
| SEQ ID | GI 260898757 NM_021571 | AGACATGTACAGCTT |
| NO: 343 | Homo sapiens caspase recruitment | ACTTTGGTGCTTTTA |
| | domain family member 18 | GCACTGAGAGTCTGC |
| | (CARD18), mRNA | TGTGATGGGAATAAA |
| SEQ ID | GI 332000013 NM_002733 | GTATACTTCCTTCAT |
| NO: 344 | Homo sapiens protein kinase AMP- | GAAGTCTCATCGCTG |
| | activated non-catalytic subunit gamma l | CTATGACCTGATTCC |
| | (PRKAG1), transcript variant 1 | CACAAGCTCCAAATT |
| SEQ ID | GI 194018737 NG_008190 | AAAGCCAGAAATTCC |
| NO: 345 | Homo sapiens acyl-CoA oxidase 1 | AGAGCATAAACATCT |
| | (ACOX1), RefSeqGene on chromosome 17 | CGTGACTGTCCTTTC |
| | | ATCTGTGGTAGGAAT |
| SEQ ID | GI 345441799 NG_029868 | GATTTTTCCTCCTTC |
| NO: 346 | Homo sapiens advanced glycosylation | AGAGATTCCTGGGAA |
| | end-product specific receptor | GCCAGAAATTGTAGA |
| | (AGER), RefSeqGene on chromosome | TTCTGCCTCTGAACT |
| SEQ ID | GI 821325145 NG_029706 | GGTAAGATAACCAAG |
| NO: 347 | Homo sapiens SH2B adaptor protein 1 | TGGGAGCCTCTAGAA |
| | (SH2B1), RefSeqGene on chromosome 16 | TGGCTCATGGGAAGT |
| | | GTGACATGAATATTG |
| SEQ ID | GI 156071494 NM_002787 | GTGCACAGAGCTCGA |
| NO: 348 | Homo sapiens proteasome subunit alpha 2 | AAACTAGCTCAACAA |
| | (PSMA2), mRNA | TACTATCTTGTGTAC |
| | | CAAGAACCCATTCCT |
| SEQ ID | GI 19718735 NM_003265 | ACTCCACCTCACTAT |
| NO: 349 | Homo sapiens toll like receptor 3 | CATGGGTTCCCAGTG |
| | (TLR3), mRNA | AGACTTTTTGATACA |
| | | TCATCTTGCAAAGAC |
| SEQ ID | GI 353703739 NG_030372 | GGGACTTAATAGACA |
| NO: 350 | Homo sapiens CD83 molecule | CATGTGCAAGAGGCT |
| | (CD83), RefSeqGene on chromosome 6 | AAAGGTGAAGCCAAA |
| | | AGTGGACAGAGATAT |
| SEQ ID | GI 195546896 NG_008330 | TCCACCTCCTTACAT |
| NO: 351 | Homo sapiens integrin subunit alpha 2 | ACTGACCTGTATTTC |
| | (ITGA2), RefSeqGene on chromosome 5 | AGAGAGCCTCTTATT |
| | | GCCATTCAGAGATCC |
| SEQ ID | GI 187473251 EU600171 | TTGGGATTTAATGCC |
| NO: 352 | Homo sapiens v-raf murine sarcoma | CTCAAATCGAGGAAC |
| | viral oncogene homolog B1 | ACAATGCACTAAAAG |
| | (BRAF) gene, complete cds | TCCTACACAGCAAAG |
| SEQ ID | GI 114731556 DQ866809 | ATATTCCAAGGATCC |
| NO: 353 | Homo sapiens endoglin | TTGAGTTCATGACAA |
| | (Osler-Rendu-Weber syndrome 1) | CAAAATCTCCCTGTT |
| | (ENG) gene, complete cds | AGCCTCTGCCTCTTC |
| SEQ ID | GI 238776813 NG_012250 | CTGAAAAGTGGAAAT |
| NO: 354 | Homo sapiens aldehyde dehydrogenase 2 family | GAACATAGTGCCTGT |
| | (mitochondrial) | CTTAGGGAGGTAATT |
| | (ALDH2), RefSeqGene on chromosome 12 | AGGGCTTAGCATGGT |
| SEQ ID | GI 224809296 NG_011498 | TTCCTCCAGAGTCCC |
| NO: 355 | Homo sapiens interleukin 23 receptor | TTATTCATGAGTTTT |
| | (IL23R), RefSeqGene on chromosome 1 | CACAGTAATCCTTTT |
| | | CACATGCACCTCAGG |
| SEQ ID | GI 1800224 U70065 Human JAK3 gene, complete cds | CACTTTAGGTAGGCT |
| NO: 356 | | GGGAACGTGATTATT |
| | | GATCCAGATTCACAT |
| | | GGGTTACAGGTTTGA |
| SEQ ID | GI 225903414 NG_011740 | CAGTGACAAACATAT |
| NO: 357 | Homo sapiens matrix metallopeptidase 1 | CCTTTCAAGACAGAA |
| | (MMP1), RefSeqGene on chromosome 11 | AGAGACAGGAGACAT |
| | | GAGTCTTTGCCGGAG |
| SEQ ID | GI 291045304 NG_016710 | GACACAAATACGAAA |
| NO: 358 | Homo sapiens ribose 5-phosphate isomerase A | GTGTTGGGGTTATTG |
| | (RPIA), RefSeqGene on chromosome 2 | GAGGTTCTTTGTGTA |
| | | GAATCTCTGGGGAGT |
| SEQ ID | GI 117606501 EF064725 | GGGCCTTCTCTATCC |
| NO: 359 | Homo sapiens interferon, beta 1, fibroblast | TTGTCTCTGTTGTTT |
| | (IFNB1) gene, complete cds | ATATCCTGCTGAAGT |
| | | GTGATTCCACTTGTG |
| SEQ ID | ref\|NG_008776.1\| | GATATGCTGTGGAAA |
| NO: 360 | Homo sapiens ciliary neurotrophic factor | TACTCCAACCCCTGT |
| | (CNTF), RefSeqGene on | GGAGAGTTTTGAATT |
| | | TAGGCTGAGAACTGA |
| SEQ ID | GI 18141132 AF424815 | CATATCATTCTGGAG |
| NO: 361 | Homo sapiens SH2 domain containing SOCS | GTGCACTTACTCTTC |
| | box protein SOCS4 mRNA, complete cds | TACAATGTGGCCGGA |
| | | TCATTTGTCATTCCT |
| SEQ ID | GI 13447389 AF361105 | CTCTGCTTCCAAACC |
| NO: 362 | Homo sapiens interleukin 9 | CTTTGCTGTTTGCAT |
| | (IL9) gene, complete cds | TCTGCCCCTTTCTTA |
| | | TTCTCTTTATACCAA |
| SEQ ID | GI 209977031 NG_008839 | TCATTTGTGTGGAAA |
| NO: 363 | Homo sapiens 24-dehydrocholesterol reductase | GTCAGAGGAAAAATG |
| | (DHCR24), RefSeqGene on chromosome 1 | ATCCAGTGGACACTT |
| | | GGGGATTATCTGTCA |
| SEQ ID | GI 336285236 NG_029146 | GCCCAAAACATAAAT |
| NO: 364 | Homo sapiens MCL1, BCL2 | GGGTGGTATGGACGC |
| | family apoptosis regulator | CGTATCACCTTACTA |
| | (MCL1), RefSeqGene on chromosome 1 | AACTGTGACATTTTG |
| SEQ ID | GI 373432598 NM_001511 | CAAATGTTCTCCAGT |
| NO: 365 | Homo sapiens C-X-C motif chemokine ligand 1 | CATTATGTTAATATT |
| | (CXCL1), transcript variant 1, mRNA | TCTGAGGAGCCTGCA |
| | | ACATGCCAGCCACTG |
| SEQ ID | GI 342349316 NG_029618 | TGCTTCAGTTAATTC |
| NO: 366 | Homo sapiens CD28 molecule | AGGTGTTGAAGGAGC |
| | (CD28), RefSeqGene on chromosome 2 | TTAGGTTTTAGAGGC |
| | | ACGTAGACTTGGTTC |
| SEQ ID | GI 19072753 AF475085 | TCATGCTTATGAGAA |
| NO: 367 | Homo sapiens mucolipin-3 | CAAAGGTACCAAGCA |
| | (MCOLN3) mRNA, complete cds | ATCTGCTATGGCAAT |
| | | CTGTCAGCACTTCTA |
| SEQ ID | GI 31096355 AY300794 | CCTTCCCTGCTTTGT |
| NO: 368 | Homo sapiens PRKC, apoptosis, WT1, regulator | AGAAATCTTCTTCAT |
| | (PAWR) gene, complete cds | TCTGCTCAGGCTCTG |
| | | ATTTCTTAGGTCAGG |
| SEQ ID | GI 300193072 NG_023297 | AGCAATTGTAACGGT |
| NO: 369 | Homo sapiens cyclin dependent kinase inhibitor 2B | TAACTGTAACGTTTT |
| | (CDKN2B), RefSeqGene on chromosome 9 | CTTTCACACACACAC |
| | | ACACACACACACACA |
| SEQ ID | GI 569026730 NG_012887 | TGTGCTCATGCCTAC |
| NO: 370 | Homo sapiens cystatin C | TTGCAGTGAAATTTT |
| | (CST3), RefSeqGene on chromosome 20 | GGAAAGTCCTTGATT |
| | | CTTTCCTTGTGTTCA |
| SEQ ID | GI 315365062 HQ384162 | GTGGAAGTGACTTGC |
| NO: 371 | Homo sapiens cell death-inducing | TTATGAGCTGTTGAC |
| | DFFA-like effector a | AAATGAAACATTGGG |
| | (CIDEA) gene, complete cds | AAACCTTGAGTCTCC |
| SEQ ID | GI 281182565 NG_016196 | ATGTTATTTTTAAGC |
| NO: 372 | Homo sapiens endothelin 1 | TCCCTTTCTTGCCAA |
| | (EDN1), RefSeqGene on chromosome 6 | TCCCTCACGGATCTT |
| | | TCTCCGATAGATGCA |
| SEQ ID | GI 269954706 NG_015999 | AGATATTGGCACCTG |
| NO: 373 | Homo sapiens enoyl-CoA hydratase and | TGAAATGAGATTTAA |
| | 3-hydroxyacyl CoA dehydrogenase | CCTGTCGCTGCTGCT |
| | (EHHADH), RefSeqGene on chrc [symbol] | TAGTGTCACTAACTC |
| SEQ ID | GI 224809296 NG_011498 | TGTCTTTCAATCCTC |
| NO: 374 | Homo sapiens interleukin 23 receptor | TGCTTATATCAGGCT |
| | (IL23R), RefSeqGene on chromosome 1 | TACCAATGTCTGTTG |
| | | GCTCAAAGCAAGCTA |
| SEQ ID | GI 259013237 NG_012931 | CTTCATAACCAGGCT |
| NO: 375 | Homo sapiens ubiquitin C-terminal hydrolase L1 | TCCTTCTGTGGGTTT |
| | (UCHL1), RefSeqGene on chromosome 4 | GGCAGTGGTTTTTGG |
| | | AAGAATCTAAAACTT |
| SEQ ID | GI 1049010967 AH003492 | ATGAGTTAGGCTTTG |
| NO: 376 | Homo sapiens beta-fibrinogen | CACTTGTAAGGAAGG |
| | (FGB) gene, complete cds | AGAAGCGTGCACAAC |
| | | CTCAAATAGCTAATA |
| SEQ ID | GI 189217842 NG_007929 | AGGATAGAGACATAT |
| NO: 377 | Homo sapiens cytochrome P450 | GGGAGTCCAGTAGAC |
| | family 2 subfamily B member 6 | ATCAATCAAACTGGA |
| | (CYP2B6), RefSeqGene on chromosome 19 [symbol] | CTCAGTTTGCACACA |
| SEQ ID | GI 594591639 NG_034041 | TTCCCTTTTCTGAGT |
| NO: 378 | Homo sapiens sorbin and SH3 domain containing 1 | CATTCTCTCTTGGCC |
| | (SORBS1), RefSeqGene on chromosome 10 | TCCTACATTCTGAAG |
| | | AGGTCATTTGCATTG |
| SEQ ID | GI 1021312320 NM_001323565 | CAGTGTGCCTACATC |
| NO: 379 | Homo sapiens cysteine dioxygenase type 1 | AATGATTCCATTGGC |
| | (CDO1), transcript variant 1, mRNA | TTACATCGAGTAGAG |
| | | AACATCAGCCATACG |
| SEQ ID | GI 207028588 NM_021970 | TCAATAAGTAGGGCA |
| NO: 380 | Homo sapiens late endosomal/lysosomal | GGCATTCTCTATGCC |
| | adaptor, MAPK and MTOR activator 3 | TGCTAGGGAAGTGTG |
| | (LAMTOR3), transcript vari [symbol] | ACTTGTTATACTCTT |
| SEQ ID | GI 209571510 NM_015000 | GTTCTTACGGCTCAA |
| NO: 381 | Homo sapiens serine/threonine kinase 38 like | AAGGACCAGACTTGG |
| | (STK38L), mRNA | CTTGGATGACTTTGA |
| | | GTCTCTGAAAGTTAT |
| SEQ ID | GI 816197633 NG_008177 | AAAATGGGTGCATTA |
| NO: 382 | Homo sapiens GATA binding protein 4 | CCCATCGTTGTGGTT |
| | (GATA4), RefSeqGene on chromosome 8 | TGGTAATGAGCTGAG |
| | | AACAAAGAAGAAACA |
| SEQ ID | GI 323510659 NG_028143 | GGAAGGGTAGGGTGG |
| NO: 383 | Homo sapiens interleukin 18 | AAAGGGTTAAGCATG |
| | (IL18), RefSeqGene on chromosome 11 | CTGTTACTGAACAAC |
| | | ATAATTAGAAGGGAA |
| SEQ ID | GI 257153325 NG_012807 | CCATACAGTGGGAGT |
| NO: 384 | Homo sapiens ST3 beta-galactoside | ATTTTTCCTTTTGGT |
| | alpha-2,3-sialyltransferase 5 | GGCGGGAATGGAGTG |
| | (ST3GAL5), RefSeqGene on chromosome 1 | AGATGGTTATAAAGA |
| | [symbol] | |
| SEQ ID | GI 6746626 AF218586 | GTACCTCTCAGCTCT |
| NO: 385 | Homo sapiens Cide-b | GAACCCCAGTGACTT |
| | (CIDEB) mRNA, complete cds | ACTCAGGTCAGTATC |
| | | TAATATAAGCTCGGA |
| SEQ ID | GI 82659110 NM_007172 | TCATCACTAGGAGCG |
| NO: 386 | Homo sapiens nucleoporin 50 | ACAAGTGCCTCATTT |
| | (NUP50), transcript variant 2, mRNA | AATTTCGGCAAGAAA |
| | | GTTGATAGCTCTGTT |
| SEQ ID | GI 2428675 AY163769 | ATGCGGGTTCTAGTT |
| NO: 387 | Homo sapiens excision repair cross- | TTTCCACATCCTTGA |
| | complementing rodent repair deficiency, | CAACAGTGGCAGTTA |
| | complementation group 3 | TCTGCTTTTTTGATT |
| | (xeroc [symbol] | |
| SEQ ID | GI 11691883 AB034990 | CTATTGGGTACACAA |
| NO: 388 | Homo sapiens HERPUD1 gene for | ATTGGTGAAGCAGGT |
| | stress protein Herp, complete cds | GTAGCCTATGGTTGC |
| | | ATCATATGGCAAAAG |
| SEQ ID | GI 238018080 NG_012203 | AATTAGTTGAAAACT |
| NO: 389 | Homo sapiens phospholipase A2 group IVA | AACACCGTGAGTTCT |
| | (PLA2G4A), RefSeqGene on chromosome 1 | TCAGTGAGGGGTGCA |
| | | GAGCTCATTTTAGCT |
| SEQ ID | GI 62953114 NM_012342 | GTCTTATCTGAACTA |
| NO: 390 | Homo sapiens BMP and activin membrane | CACTTACTGAACAGC |
| | bound inhibitor | TTGAAGGCCTTTTGA |
| | (BAMB1), mRNA | GTTCTGCTGGACAGG |
| SEQ ID | GI 119220595 NM_016260 | CAAGTGACAGATAGT |
| NO: 391 | Homo sapiens IKAROS family zinc finger 2 | AAGAAAAGCTTCTTG |
| | (IKZF2), transcript variant 1, mRNA | AGCAGGTGGAGGTCA |
| | | CTGAATCCCCTACTA |
| SEQ ID | GI 4885488 NM_005372 | CAGTTAAGTTTGGGA |
| NO: 392 | Homo sapiens MOS proto-oncogene, | AAGTGTCTCAAGTAC |
| | serine/threonine kinase | TCACTAGATGTTGTG |
| | (MOS), mRNA | AACGGCCTGCTCTTC |
| SEQ ID | GI 343790995 NG_029773 | AAGCAAGCCTCCTCA |
| NO: 393 | Homo sapiens activating transcription factor 6 | TTGTCTAGAGGGAGA |
| | (ATF6), RefSeqGene on chromosome 1 | TTTTATCTCATCCTT |
| | | CAAGGTTGCTTGTTG |
| SEQ ID | GI 194595503 NG_008308 | GAGCATTTGGAAGGG |
| NO: 394 | Homo sapiens gap junction protein alpha l | GATAGGGAAGCACAT |
| | (GJA1), RefSeqGene on chromosome 6 | AAAGATGATATGCTC |
| | | AGCTTTACTGTCGTT |
| SEQ ID | GI 1031698869 NG_050687 | CCTTTTCTCTTCCCC |
| NO: 395 | Homo sapiens HOP homeobox | GTCTTCTCTCAGCCA |
| | (HOPX), RefSeqGene on chromosome 4 | CATCTCTCTTATCCT |
| | | ATCTTGGTGAAGAAG |
| SEQ ID | GI 385275063 NG_032144 | GCCAATGCAAAGAGA |
| NO: 396 | Homo sapiens transient receptor potential | GAAACTGGATGAACC |
| | cation channel subfamily V member 3 | ATATCACATTCTCAG |
| | (TRPV3), RefSeqGene on [symbol] | CCTGTCTCTAAGGAG |
| SEQ ID | GI 153792589 NM_001017963 | GAGCTGTTAAGGTAC |
| NO: 397 | Homo sapiens heat shock protein 90 | TACACATCTGCCTCT |
| | alpha family class A member 1 | GGTGATGAGATGGTT |
| | (HSP90AA1), transcript variant 1, n [symbol] | TCTCTCAAGGACTAC |
| SEQ ID | GI 117606501 EF064725 | AGCAGTTTAAGTCCA |
| NO: 398 | Homo sapiens interferon, beta 1, fibroblast | GTTTCAAACCAAGCA |
| | (IFNB1) gene, complete cds | AAAAGCCAGGAAACA |
| | | TAGGAGAAAGGGAAG |
| SEQ ID | GI 237874277 NG_012191 | GACCTGTCCTGTGAA |
| NO: 399 | Homo sapiens BCL2 associated X, | ATCACACTCTGTCAT |
| | apoptosis regulator | TTCCACAATATCCTC |
| | (BAX), RefSeqGene on chromosome 19 | TTGGTCACACTGTTT |
| SEQ ID | GI 225579089 NG_011672 | CTCATTCAGAGTTTC |
| NO: 400 | Homo sapiens decorin | GAATGCACCAAAAGG |
| | (DCN), RefSeqGene on chromosome 12 | GTGTTTCCAAAACAT |
| | | GATATGGGAAGGAGG |
| SEQ ID | GI 224809324 NG_011504 | TTGCAGTTGACAAAC |
| NO: 401 | Homo sapiens NAD | TGAAATTATCAGGAT |
| | (P)H quinone dehydrogenase 1 | CTCTGAGAAAGGGAC |
| | (NQO1), RefSeqGene on chromosome 16 | TAAGCTGGGGATTGG |
| SEQ ID | GI 538917457 NM_005896 | GGAAGTTCTGGTGTC |
| NO: 402 | Homo sapiens isocitrate dehydrogenase | ATAGATATCCCGTTT |
| | (NADP | TGTGAGGTAGAGCTG |
| | (+)) 1, cytosolic | TGCATTAAACTTGCA |
| | (IDH1), transcript variant 1, mRNA | |
| SEQ ID | ref\|NG_047051.1\| | AAAAAAATTCACAGC |
| NO: 403 | Homo sapiens growth differentiation factor 9 | CTGGGCAACAGACAG |
| | (GDF9), RefSeqGene | AGTTTTGCAGTTACC |
| | | CATGAAAAAAATCCC |
| SEQ ID | GI 221139813 NG_009361 | ACAGAAGATGAGGGA |
| NO: 404 | Homo sapiens BCL2, apoptosis regulator | ATCTTTGGAAATTTA |
| | (BCL2), RefSeqGene on chromosome 18 | GGCGACAGAGGCCAG |
| | | ACATACCAATAAACG |
| SEQ ID | GI 209447066 NG_008725 | TTCTCGTGACTCTCC |
| NO: 405 | Homo sapiens paraoxonase 2 | TTTGTATCTAGAACA |
| | (PON2), RefSeqGene on chromosome 7 | CAGTGTCTTTCCCCA |
| | | AAACCTGGGTCATAT |
| SEQ ID | GI 29542 X06596 Human mRNA for | CTTTAAGAAAATGCA |
| NO: 406 | complement component Cls | AAGAAGTGAAAGTGG |
| | | AGAAACCCACAGCAG |
| | | ATGCAGAGGCCTATG |
| SEQ ID | GI 189181756 NM_001710 | GACTTGCTATACATT |
| NO: 407 | Homo sapiens complement factor B | GGCAAGGATCGCAAA |
| | (CFB), mRNA | AACCCAAGGGAGGAT |
| | | TATCTGGATGTCTAT |
| SEQ ID | GI 384367978 NM_021797 | GGGCATAAACCTGAC |
| NO: 408 | Homo sapiens chitinase, acidic | CTGGTCTATATTCCC |
| | (CHIA), transcript variant 2, mRNA | TAGAGTTCCAGTCTC |
| | | TTTTGCTTAGGACAT |
| SEQ ID | GI 83267865 NM_001037494 | CCTTGCTAAGGGAAC |
| NO: 409 | Homo sapiens dynein light chain LC8-type 1 | ATCTCGATGTTTGAA |
| | (DYNLL1), transcript variant 1, mRNA | CCTTTGTTGTGTTTT |
| | | GTACAGGGCATTCTC |
| SEQ ID | GI 237858596 NG_012143 | TTTTTCGTTTTTTTT |
| NO: 410 | Homo sapiens nuclear receptor | TABACAGAGTCTTBC |
| | subfamily 0 group B member 2 | TCTGTCTABAGCAAG |
| | (NROB2), RefSeqGene on chromosome 1 | GCAGGAGGGCAGTAA |
| SEQ ID | GI 343962600 NG_029791 | GTATTCTCTGCCCTC |
| NO: 411 | Homo sapiens TNF receptor superfamily member 1B | ATAGGACACTCTAGT |
| | (TNFRSF1B), RefSeqGene on chromosome 1 | AGAGCAGCTGGGTGC |
| | | TAATAACAGAAACAC |
| SEQ ID | GI 588480544 NG_029714 | GTTGGATGACTGGAT |
| NO: 412 | Homo sapiens tissue factor pathway inhibitor | AGTCAAAGGAAGACT |
| | (TFPI), RefSeqGene on chromosome 2 | GAAGCTAGCATATGT |
| | | CTCTCCACCACATCT |
| SEQ ID | GI 984290537 NM_001319196 | ACAGGGATGACCTGA |
| NO: 413 | Homo sapiens S100 calcium binding protein A8 | AGAAATTGCTAGAGA |
| | (S100A8), transcript variant 1, mRNA | CCGAGTGTCCTCAGT |
| | | ATATCAGGAAAAAGG |
| SEQ ID | GI 238776812 NG_012249 | CTACTCTGGGACACT |
| NO: 414 | Homo sapiens aldehyde | GACTTGCCAGAATCC |
| | dehydrogenase 1 family member A1 | TCTTGTATCCTTCTT |
| | (ALDH1A1), RefSeqGene on chromosome 9 | CATCCTGACTTTCAT |
| SEQ ID | GI 973353146 NG_016274 | TCTGGGAACCAGATG |
| NO: 415 | Homo sapiens hepatocyte growth factor | CAAGTAAGCTGAATG |
| | (HGF), RefSeqGene on chromosome 7 | AGAATTACTGCCGAA |
| | | ATCCAGATGATGATG |
| SEQ ID | GI 338827643 NG_029228 | TGGTTAAGAAGACCA |
| NO: 416 | Homo sapiens OS9, endoplasmic reticulum lectin | CTGGAGGAGTAGACA |
| | (OS9), RefSeqGene on chromosome 12 | GATTACACGGACAGG |
| | | AAAGAAGCAAAGAAT |
| SEQ ID | GI 332078493 NM_002559 | AGCTGAGAACTTCAC |
| NO: 417 | Homo sapiens purinergic receptor P2X 3 | TATTTTCATCAAGAA |
| | (P2RX3), mRNA | CAGCATCCGTTTCCC |
| | | CCTCTTCAACTTTGA |
| SEQ ID | GI 90657251 DQ451402 | CATTGAGGAGGTAGA |
| NO: 418 | Homo sapiens heat shock 70 kDa protein 1A | TTAGGGGCCTTTCCA |
| | (HSPA1A) gene, complete cds | AGATTGCTGTTTTTG |
| | | TTTTGGAGCTTCAAG |
| SEQ ID | GI 32187528 AY324876 | GTCCATTCGTTCACT |
| NO: 419 | Homo sapiens lactoperoxidase | CATCCGTTTATTCAG |
| | (LPO) gene, complete cds | TAAATAGACATTGAC |
| | | CACCCCATACCAGGC |
| SEQ ID | GI 340523104 NG_029469 | ATTCCAGGTGTTCTA |
| NO: 420 | Homo sapiens heat shock protein family A | AATCTTGCTAAGAAC |
| | (Hsp70) member 9 | CCGGTGGGAATAAAG |
| | (HSPA9), RefSeqGene on chromosome 5 | GTTGCATGTACAGAG |
| SEQ ID | GI 224809271 NG_011477 | TATGATCACTGTACC |
| NO: 421 | Homo sapiens TNF superfamily member 4 | ACAGACAGTGCTTGT |
| | (TNFSF4), RefSeqGene on chromosome 1 | GTCTCCAAATTCCAG |
| | | AAGTGTAGCTAGTGT |
| SEQ ID | GI 1020158924 NM_002895 | CTGACAGTGGGAGTC |
| NO: 422 | Homo sapiens RB transcriptional | TTCGAAGAGATATGC |
| | corepressor like 1 | AACCATTGTCTCCAA |
| | (RBL1), transcript variant 1, mRNA | TTTCTGTCCATGAAC |
| SEQ ID | GI 257743127 NG_012865 | GGACTCCCTCTTCAT |
| NO: 423 | Homo sapiens amylo-alpha-1, | ACCTCTTGGACTCTA |
| | 6-glucosidase, 4-alpha-glucanotransferase | ACATCCTGCTCCATA |
| | (AGL), RefSeqGene on chromoson [symbol] | CTACCTATTACCCTT |
| SEQ ID | GI 219521908 NG_009245 | ACTAAACTGGCAGCT |
| NO: 424 | Homo sapiens bone morphogenetic | TCACATTTTCTTGCT |
| | protein receptor type 1B | CATTCTTCTCTCTAT |
| | (BMPR1B), RefSeqGene on chromosome 4 | GCACACAAGGGGTAA |
| SEQ ID | GI 226491119 NG_011810 | CCTGGAGAATCCCTT |
| NO: 425 | Homo sapiens 1,4-alpha-glucan branching enzyme 1 | CTTTTGTAGCAGCTC |
| | (GBE1), RefSeqGene on chromosome 3 | TGTGTCTTCAAGGGC |
| | | ATCATTATGTTATCC |
| SEQ ID | GI 218749810 NG_009188 | TTTGGCAAAACTGAT |
| NO: 426 | Homo sapiens phosphoglycerate dehydrogenase | TGCATGGAGCACTTC |
| | (PHGDH), RefSeqGene on chromosome 1 | TGTTGAACAGGCAAT |
| | | TCAAGCATTTATGTG |
| SEQ ID | GI 225579099 NG_011677 | AAAGGTCTGGATTGG |
| NO: 427 | Homo sapiens pyruvate kinase, liver and RBC | AGAGACATTTGGGAG |
| | (PKLR), RefSeqGene on chromosome 1 | TTGGCTACATATAGA |
| | | TGGTATCTCAAGCCA |
| SEQ ID | GI 189909140 EU779708 | AATATCCTCTGAACT |
| NO: 428 | Homo sapiens protein tyrosine phosphatase-2 | GTGCAGATCCTACCT |
| | (PTPN11) mRNA, complete cds | CTGAAAGGTGGTTTC |
| | | ATGGACATCTCTCTG |
| SEQ ID | GI 325910867 NM_004874 | CCATTATCCTTATGG |
| NO: 429 | Homo sapiens BCL2 associated athanogene 4 | AGATGGTAATCGTAG |
| | (BAG4), transcript variant 1, mRNA | TGTTCCACAATCAGG |
| | | ACCGACTGTACGACC |
| SEQ ID | GI 157427676 NM_004911 | GAGAAATTGTTGGCG |
| NO: 430 | Homo sapiens protein disulfide | TAGATTTGGCTTCTG |
| | isomerase family A member 4 | GTATGTGTTTCTGAT |
| | (PDIA4), mRNA | TGCTTCCTGTTGAGC |
| SEQ ID | GI 22859017 AY148100 Homo sapiens | TGTGTCTGCTGTTGT |
| NO: 431 | colony stimulating factor 3 receptor | TATTATCATTAGCCA |
| | (granulocyte) | GACCAGGTTAAGCTC |
| | (CSF3R) gene, complete cds | TCTCTCTCACACACA |
| SEQ ID | GI 334848111 NG_029036 | TGACTATCAGAGTTT |
| NO: 432 | Homo sapiens promyelocytic leukemia | GGCTTGTGTTTGGCT |
| | (PML), RefSeqGene on chromosome 15 | GATGAGCAAACCTGG |
| | | AATGAATCAGTGATA |
| SEQ ID | GI 224809246 NG_011451 | CTAGGATGTGTTGAA |
| NO: 433 | Homo sapiens integrin subunit alpha X | GGGCACACTGTCTGC |
| | (ITGAX), RefSeqGene on chromosome 16 | ATATAGTGAGTGATT |
| | | GAAGAACATGTTGGG |
| SEQ ID | GI 227908772 NG_011947 | ATGGACCTTTAATTT |
| NO: 434 | Homo sapiens tryptophan hydroxylase 1 | GCAGACACCAACTAG |
| | (TPH1), RefSeqGene on chromosome 11 | TTTCAACATGCTCTG |
| | | CCTTACTCTCCAAGT |
| SEQ ID | GI 130509310 NM_022073 | CTGTGGTTGGCAGTA |
| NO: 435 | Homo sapiens egl-9 family | TGACCAGAATCATGG |
| | hypoxia inducible factor 3 | AATTTGCTAGAACTG |
| | (EGLN3), transcript variant 1, mRNA | TGGAAGCTTCTACTC |
| SEQ ID | GI 908212430 NM_000412 | CAAACATCCTCTAAA |
| NO: 436 | Homo sapiens histidine rich glycoprotein | GCCAGACAATCAGCC |
| | (HRG), mRNA | CTTTCCTCAATCAGT |
| | | CTCTGAATCATGTCC |
| SEQ ID | GI 31657141 NM_181501 | ATTTGAAATGGAAAT |
| NO: 437 | Homo sapiens integrin subunit alpha 1 | GTCTCAGACTGGCTT |
| | (ITGA1), mRNA | CAGTGCTCATTATTC |
| | | ACAGGACTGGGTCAT |
| SEQ ID | GI 164698262 AK314515 | CCAAGCCTTCCTGCT |
| NO: 438 | Homo sapiens cDNA, FLJ95334, | GTAGACACAAGCTAC |
| | Homo sapiens protein inhibitor | ATTAATACCTCCCTC |
| | of activated STAT, 1 | ATCCAAGACTATAGG |
| | (PIAS1), mRNA | |
| SEQ ID | GI 385198082 NM_005226 | TAGCCAGCTGGAGAT |
| NO: 439 | Homo sapiens sphingosine-1- | AATTTACCAGGGAAT |
| | phosphate receptor 3 | GTTTCAAAGCCCTAA |
| | (S1PR3), mRNA | CCTTGAAGTTTGGAA |
| SEQ ID | GI 84872755 DQ355026 | GAATGGGAGAAGTAT |
| NO: 440 | Homo sapiens caspase 8, apoptosis- | AGACACATCTCCACT |
| | related cysteine peptidase | CCACCTCAAGTGAAC |
| | (CASP8) gene, complete cds | TATCTGGCTTTGTGT |
| SEQ ID | GI 584458486 NG_012245 | TGTCAGCTGGAAGCT |
| NO: 441 | Homo sapiens N-acetyltransferase 1 | TTTTGATGTGCTACA |
| | (NAT1), RefSeqGene on chromosome 8 | GCAAAAGGCTAAGAA |
| | | ATGGCGTATTGAAAT |
| SEQ ID | GI 338827643 NG_029228 | TCCTTGACTTGTTTC |
| NO: 442 | Homo sapiens OS9, endoplasmic reticulum lectin | TGACTTTAAAGGGGA |
| | (OS9), RefSeqGene on chromosome 12 | TGCTCCTGAAGTTTC |
| | | ACTGTTAGCGTGATG |
| SEQ ID | GI 237858601 NG_012145 | GCTATTATACAGAAG |
| NO: 443 | Homo sapiens TNF receptor superfamily member 10b | GTGAGGCCAAAAATG |
| | (TNFRSF10B), RefSeqGene on chromosome 8 | CTGGAGACACTGCTC |
| | | CTGAATCTCAGTTTT |
| SEQ ID | GI 33333853 AF546700 | TGCTTAGTTACTGGT |
| NO: 444 | Homo sapiens androgen regulated a/b hydrolase II | TTTCTCCATTGCATT |
| | (ABHD2) mRNA, complete cds | GTTAGGCATGGTGAC |
| | | AAGTGACAGAGTTCT |
| SEQ ID | GI 189217867 NG_007938 | TGTATCCATGGCAAT |
| NO: 445 | Homo sapiens cytochrome P450 | TTGCTTTCCCCTCAC |
| | family 3 subfamily A member 5 | TGAACTGAGATCAGA |
| | (CYP3A5), RefSeqGene on chromosome 7 | ATGTTACTCTGTTGG |
| SEQ ID | GI 374349248 NG_032045 | TGAGGCAGATTAGAC |
| NO: 446 | Homo sapiens phospholipase A2 group V | TTAAGACTGTAAGGA |
| | (PLA2G5), RefSeqGene on chromosome 1 | GTGGTTAAGGACTAG |
| | | GGCAGAGCAAAACAC |
| SEQ ID | GI 893579206 NG_007504 | AGGTTAAAGAAAACA |
| NO: 447 | Homo sapiens sonic hedgehog | AGAAACACAATCAGA |
| | (SHH), RefSeqGene on chromosome 7 | CCACAGAAAAGCCAG |
| | | GCAGAAAAGGGTTCG |
| SEQ ID | GI 296434305 NG_021272 | ATAAGGAGCTGATTC |
| NO: 448 | Homo sapiens signal transducer | TGCTAGGAGCAAGGT |
| | and activator of transcription 6 | CTTATCTCCACGATA |
| | (STAT6), RefSeqGene on chromosome 12 | TTCCAAAAGGTCAGG |
| SEQ ID | GI 5821735 AF155233 | TTTATTGGCATCTTA |
| NO: 449 | Homo sapiens snail zinc finger protein | TTCGACTAAGGCTAC |
| | (SNAIl) gene, complete cds | CCATTTCTCTTCCTT |
| | | CGTGCACCAATTGCT |
| SEQ ID | GI 283806588 NG_016341 | AAAAAAGCAACAGAA |
| NO: 450 | Homo sapiens cyclin dependent kinase inhibitor 1B | ACCTATCCTCACTGC |
| | (CDKN1B), RefSeqGene on chromosome 12 | CCTCCCCAGTCTCTC |
| | | TTAAAGTTGGAATTT |
| SEQ ID | GI 1146072968 NG_011674 | CTGGCAAGACAGCAT |
| NO: 451 | Homo sapiens glycine amidinotransferase | AACTCTGCATCTGCT |
| | (GATM), RefSeqGene on chromosome 15 | GTAACCCAGTGTTAA |
| | | ATTTTCAAAGGCTTC |
| SEQ ID | GI 1059792660 AH007003 | AAGCAACCTTGCTCT |
| NO: 452 | Homo sapiens MET proto-oncogene, | ACCTTCCCTCTGATA |
| | intron 2, 3′ end | GGGACATTTAGTCAT |
| | | CTTTGCATGTCTACT |
| SEQ ID | GI 334883185 NG_029065 | AGTAAGCAAATGGGC |
| NO: 453 | Homo sapiens cAMP responsive element modulator | AAAAAATGAAATGTA |
| | (CREM), RefSeqGene on chromosome 10 | AATGTGCAACCCGGA |
| | | ATTGGCAACTCTGTG |
| SEQ ID | GI 665505917 NM_006145 | ATTCCCCAGACATCA |
| NO: 454 | Homo sapiens DnaJ heat shock protein family | AGAACCGTACTTGAG |
| | (Hsp40) member B1 | CAGGTTCTTCCAATA |
| | (DNAJB1), transcript variant 1, mRNA | TAGCTATCTGAGCTC |
| SEQ ID | GI 429535870 NG_033021 | CTTTGGAGATTAAGG |
| NO: 455 | Homo sapiens interleukin 17A | CCCCTCAGAGATCAA |
| | (IL17A), RefSeqGene on chromosome 6 | CAGACCAACATTTTT |
| | | CTCTTCCTCAAGCAA |
| SEQ ID | GI 342349297 NG_029616 | AATCACCCGTCATTC |
| NO: 456 | Homo sapiens protein kinase C zeta | GGAGGTTTGTGTATG |
| | (PRKCZ), RefSeqGene on chromosome 1 | GGGAAGATCAGCTCA |
| | | GCATTATTTTTACAA |
| SEQ ID | GI 130509310 NM_022073 | CTACTCGGCCAGCGG |
| NO: 457 | Homo sapiens egl-9 family | TTTACCTGATAGATT |
| | hypoxia inducible factor 3 | CGGTAATACTATCAA |
| | (EGLN3), transcript variant 1, mRNA | GAGAAGAGCCTAGGA |
| SEQ ID | GI 219555683 NM_176891 | TCTGTTCTTTGTGTT |
| NO: 458 | Homo sapiens interferon epsilon | CAGTCTCACAGAAAA |
| | (IFNE), mRNA | ACTGAGCAAACAAGG |
| | | AAGACCCTTGAACGA |
| SEQ ID | GI 211904170 NG_008937 | TGGGTCAAGAAGTAC |
| NO: 459 | Homo sapiens CD44 molecule | TTCTCCTTCATCTTC |
| | (Indian blood group) | CTTTGATGTCGGTAA |
| | (CD44), RefSeqGene on chromosome 11 | CTCATCCTTTCTGCA |
| SEQ ID | GI 270047497 NG_016131 | AGCACATGTGTGAGA |
| NO: 460 | Homo sapiens connective tissue growth factor | AAAGCAGAGAAACAA |
| | (CTGF), RefSeqGene on chromosome 6 | CAAGGTATCTGATGC |
| | | TTCTGAGAACACGAG |
| SEQ ID | GI 341865611 NG_029556 | TCCATCCTGAGAGGG |
| NO: 461 | Homo sapiens growth factor | ATTAATAGGACATGT |
| | receptor bound protein 2 | GTAGAAACAGACTTA |
| | (GRB2), RefSeqGene on chromosome 17 | CACCTCCTCTCTCCC |
| SEQ ID | GI 210032106 NG_008851 | CAGTAATAGACCTGA |
| NO: 462 | Homo sapiens interleukin 1 beta | AGCTGGAACCCATGT |
| | (ILIB), RefSeqGene on chromosome 2 | CTAATAGTGTCAGGT |
| | | CCAGTGTTCTTAGCC |
| SEQ ID | GI 82880650 NM_004051 | CTGTGGAAATACTTC |
| NO: 463 | Homo sapiens 3-hydroxybutyrate dehydrogenase 1 | ACATTTGCATTCCAG |
| | (BDHl), transcript variant 1, mRNA | GACTGTTTTGGATCC |
| | | ATTTGCCCGAGAATC |
| SEQ ID | GI 46488017 AY601851 | GAAGAGTCTCATACA |
| NO: 464 | Homo sapiens mutS homolog 2, | ACGGAAGAGGCGTTA |
| | colon cancer, nonpolyposis type 1 | TATGAGACACCAAAG |
| | (E. coli) | TCCACGTTGAGTCTT |
| | (MSH2) gene, complete cds | |
| SEQ ID | GI 9837432 AF287892 | GTTTTCCTGCACTTC |
| NO: 465 | Homo sapiens sialic acid binding | AGTCTATGTCTGTGT |
| | immunoglobulin-like lectin 8 long | GTCAGTCCCTCTCAC |
| | splice variant | TAGTCTCTTTCTGTG |
| | (Siglec8) gene, complete cds | |
| SEQ ID | GI 224809353 NG_011513 | TCTCTCGTCCTCCTC |
| NO: 466 | Homo sapiens ATP binding cassette | TCTTAGAAGCTTTGA |
| | subfamily B member 1 | TGTCATTCTGTTCCT |
| | (ABCB1), RefSeqGene on chromosome 7 | TCCGTGTCTCTCTAG |
| SEQ ID | GI 260593637 NG_013005 | GGCACTCAAAGTATT |
| NO: 467 | Homo sapiens calcitonin receptor | TGTCGACTGAAAAAA |
| | (CALCR), RefSeqGene on chromosome 7 | ACTGATGATGTTTGG |
| | | GCTGCTGACTACTGC |
| SEQ ID | GI 269954724 NG_016003 | CTGCATCTTCAATTT |
| NO: 468 | Homo sapiens interferon alpha and | TTGTTGTCCCTAGCC |
| | beta receptor subunit 2 | CTCCAAATGCTGGAA |
| | (IFNAR2), RefSeqGene on chromosome 21 | TAAGTGGAAATGGAC |
| SEQ ID | GI 167614492 NM_016583 | CTGAGACCTCTAAGA |
| NO: 469 | Homo sapiens BPI fold containing family A member 1 | AGTCCAGATACTAAG |
| | (BPIFA1), transcript variant 1, mRNA | AGCAAAGATGTTTCA |
| | | AACTGGGGGCCTCAT |
| SEQ ID | GI 119703747 NM_001432 | ATGTATCCCAGGAGA |
| NO: 470 | Homo sapiens epiregulin | GTCCAGTGATAACTG |
| | (EREG), mRNA | CACAGCTTTAGTTCA |
| | | GACAGAAGACAATCC |
| SEQ ID | GI 321267517 NG_028118 | TCAGTAAGGAGTGTC |
| NO: 471 | Homo sapiens arrestin beta 1 | TTGGGAATGCAGACT |
| | (ARRB1), RefSeqGene on chromosome 11 | GCTTGAGGACAAAAA |
| | | AACTAGGGTGAAAAG |
| SEQ ID | GI 209977070 NG_008850 | CATATCCTACAGAAG |
| NO: 472 | Homo sapiens interleukin l alpha | ACCAGGCTTCTCTCT |
| | (ILIA), RefSeqGene on chromosome 2 | GGTCCTTGGTAGAGG |
| | | GCTACTTTACTGTAA |
| SEQ ID | GI 297515495 NG_021405 | CACCCCTCTTCTTGC |
| NO: 473 | Homo sapiens MHC class 1 | TATCTCCTATACCAA |
| | polypeptide-related sequence B | TAAATACGAAGGGCT |
| | (MICB), RefSeqGene on chromosome 6 | GTGGAAGATCAGAGC |
| SEQ ID | GI 148298657 NM_002089 | AATGGCCAGTGAGAT |
| NO: 474 | Homo sapiens C-X-C motif chemokine ligand 2 | CAATGTGACGGCAGG |
| | (CXCL2), mRNA | GAAATGTATGTGTGT |
| | | CTATTTTGTAACTGT |
| SEQ ID | GI 209413721 NM_021068 | TATCCATCTCAAGTA |
| NO: 475 | Homo sapiens interferon alpha 4 | GCCTAGCAATATTTG |
| | (IFNA4), mRNA | CAACATCCCAATGGC |
| | | CCTGTCCTTTTCTTT |
| SEQ ID | GI 21105788 AF512686 | ACCTTTGTTGCCCAG |
| NO: 476 | Homo sapiens interleukin 12B | CAAAGCCAAAACACT |
| | (natural killer cell stimulatory | GAGTTCTGGCCAATG |
| | factor 2, cytotoxic lymphocyte | AGATATAAGAAAATT |
| | maturation factor | |
| SEQ ID | GI 1025608659 NG_047042 | ATCTTTGCAGCTACC |
| NO: 477 | Homo sapiens cell division cycle 42 | ATAATCACCCTCTCT |
| | (CDC42), RefSeqGene on chromosome 1 | CTCTCTTTGCCTGTG |
| | | CCTTCCATAGAATAA |
| SEQ ID | GI 343488501 NG_029726 | GCCACATTCTCTAAG |
| NO: 478 | Homo sapiens dynamin 1 | GAAGAACACAGCAGA |
| | (DNM1), RefSeqGene on chromosome 9 | ACAATAGCATCTGTT |
| | | TTGTTTCTGCATGGC |
| SEQ ID | GI 335334929 NG_012849 | TTTTAAGGGAAGCCA |
| NO: 479 | Homo sapiens phosphoinositide-3- | GTGGCAGATGGAGAG |
| | kinase regulatory subunit 1 | CCATCAGATGAAACT |
| | (PIK3R1), RefSeqGene on chromosome 5 | GTTATTAATCACACC |
| SEQ ID | GI 237874184 NG_012166 | ATCAGTCATTCAGGA |
| NO: 480 | Homo sapiens T-box 21 | GGGTCTTACTGAAAG |
| | (TBX21), RefSeqGene on chromosome 17 | CTCTCATGTTTCCCC |
| | | AAAAGTAGTGAAGGA |
| SEQ ID | ref\|NG_029387.2\| | CGATCTCTGTCACAA |
| NO: 481 | Homo sapiens V-set pre-B cell | CTGTTCAATTCTGTC |
| | surrogate light chain 1 | CTAGTAGCAAGAAAG |
| | (VPREB1), | CAGCCACAGACAAAC |
| SEQ ID | GI 219521908 NG_009245 | GACGCAGGTTGGCAA |
| NO: 482 | Homo sapiens bone morphogenetic | CTAGATGTGACGGGG |
| | protein receptor type 1B | CACAACAAGGTAGGA |
| | (BMPR1B), RefSeqGene on chromosome 4 | GGGGTTTTTTGGTGT |
| SEQ ID | GI 254826724 NG_012488 | ACCACAGCTTAACAA |
| NO: 483 | Homo sapiens prostaglandin E synthase 2 | CATAGATTCTGAAAT |
| | (PTGES2), RefSeqGene on chromosome 9 | CAGCGATCTGGGTTT |
| | | GAGGTGTGATTCACC |
| SEQ ID | GI 256600243 NG_012682 | GGACAGCTAGAAAAC |
| NO: 484 | Homo sapiens versican | ACAAAATCTTGTAGG |
| | (VCAN), RefSeqGene on chromosome 5 | TCATTGCACCTATCT |
| | | CAGCCATAGGTGCAG |
| SEQ ID | GI 68165954 DQ086801 | TTTCCATTCTACAGA |
| NO: 485 | Homo sapiens suppressor of | AGGGAAAACTGAGTA |
| | cytokine signaling 1 | AATCTGCCTGTGGCC |
| | (SOCS1) gene, complete cds | ATTCCTGTCAAAGAC |
| SEQ ID | GI 117606467 EF064708 | CAAGGATTGACAGCC |
| NO: 486 | Homo sapiens tumor necrosis factor | ATCATCAGAAGCTCG |
| | (ligand) family, member 10 | GAAGAAACAAGGAAG |
| | (TNFSF10) gene, complete cds | GATTGATTCTATGCA |
| SEQ ID | GI 83699688 DQ323999 | TCATGAAACGAGATT |
| NO: 487 | Homo sapiens TNF receptor-associated factor 4 | CAAAGAAAGGCTGCG |
| | (TRAF4) gene, complete cds | ACTTGCCCAGGGTCA |
| | | CATAGTTACTTAAAG |
| SEQ ID | GI 300244503 NG_023318 | GGCCAGACTGCCTGA |
| NO: 488 | Homo sapiens Fc fragment of IgG receptor lib | GTTCAAATCTAGTTC |
| | (FCGR2B), RefSeqGene on chromosome 1 | TACAGCATACCAACT |
| | | ATATGACCCAAGTGA |
| SEQ ID | GI 224809428 NG_011537 | ATGTTGTCCGTGGAA |
| NO: 489 | Homo sapiens myosin heavy chain 3 | ACAAACCAATTTCTA |
| | (MYH3), RefSeqGene on chromosome 17 | CAATGTCCGTGCAGT |
| | | CCTAAGCATTTTTCC |
| SEQ ID | GI 335334929 NG_012849 | ATTTTTCTCTCAGGC |
| NO: 490 | Homo sapiens phosphoinositide-3- | TAACCAAAACCATGT |
| | kinase regulatory subunit 1 | TGAAATGAAAACCAC |
| | (PIK3R1), RefSeqGene on chromosome 5 | CTTGGAACTGGCCTT |
| SEQ ID | GI 291045304 NG_016710 | CTAGGAGACCTTATG |
| NO: 491 | Homo sapiens ribose 5-phosphate isomerase A | TTCTGCTTGTAGGCT |
| | (RPIA), RefSeqGene on chromosome 2 | GAAGGTAGATGTTGC |
| | | TAATCACAGCACTGT |
| SEQ ID | GI 343962631 NM_002037 | CGAAAAGATGCTGAG |
| NO: 492 | Homo sapiens FYN proto-oncogene, | CGACAGCTATTGTCC |
| | Src family tyrosine kinase | TTTGGAAACCCAAGA |
| | (FYN), transcript variant 1, mRNA | GGTACCTTTCTTATC |
| SEQ ID | GI 807201065 NM_001008397 | GTGTCATACATATTG |
| NO: 493 | Homo sapiens glutathione peroxidase 8 | CCTTGAAAGAAAGGA |
| | (putative) | CATTCAGGTGTCCCT |
| | (GPX8), transcript variant 1, mRNA | TCGGGGTTCTGTAAC |
| SEQ ID | GI 297374833 NM_006184 | CCAACAGGTTTAACC |
| NO: 494 | Homo sapiens nucleobindin 1 | CCAAGACCTTCTTCA |
| | (NUCB1), mRNA | TACTGCATGATATCA |
| | | ACAGTGATGGTGTCC |
| SEQ ID | GI 209862906 NM_006475 | ATATCGTACAGCTGT |
| NO: 495 | Homo sapiens periostin | CTGCATTGAAAATTC |
| | (POSTN), transcript variant 1, mRNA | ATGCATGGAGAAAGG |
| | | GAGTAAGCAAGGGAG |
| SEQ ID | GI 31559177 AY309922 | TTAGTGGTATTCACT |
| NO: 496 | Homo sapiens BH3 interacting domain death agonist | GTGTATGTGACACAC |
| | (BID) gene, complete cds | CAGCATGGGTTAGTG |
| | | GTATTCCTCCATGAT |
| SEQ ID | GI 224282141 NG_009873 | CTCTTCCTCCCCTTC |
| NO: 497 | Homo sapiens CREB binding protein | TTAAACACGAACAGG |
| | (CREBBP), RefSeqGene on chromosome 16 | CTAAATTGTCCATTT |
| | | CTCCAGCAGTTTTGA |
| SEQ ID | GI 209870110 NM_013943 | TCTCACTCATTGCAC |
| NO: 498 | Homo sapiens chloride intracellular channel 4 | TTCCTGGAGTTAAAT |
| | (CLIC4), mRNA | TTTCCAACAGCCATG |
| | | TTGAGGAATAGCACT |
| SEQ ID | GI 156142193 NM_005880 | CTATGCCCATCTTAT |
| NO: 499 | Homo sapiens DnaJ heat shock protein family | AGTCTTGGAAGACAA |
| | (Hsp40) member A2 | AAGGCTGGTAGAGAC |
| | (DNAJA2), mRNA | AGTCTTCAGTGGCTT |
| SEQ ID | GI 1073857156 NM_016930 | ACCTCCAGGAACTGT |
| NO: 500 | Homo sapiens syntaxin 18 | AAACAGGAACACCTA |
| | (STX18), transcript variant 1, mRNA | TGTGTAACTGAAGGC |
| | | ATGAGACATCTGTAC |
| SEQ ID | GI 1002095861 NG_046750 | GATGTCATTCACAAG |
| NO: 501 | Homo sapiens cytochrome c oxidase subunit 8A | TATAAGCCTTGGAGG |
| | (COX8A), RefSeqGene on chromosome 11 | CGGAACATAACTCGA |
| | | AGAAGAGCCCTTTTG |
| SEQ ID | GI 584593316 NG_033952 | AGGAAGCAAGACGTA |
| NO: 502 | Homo sapiens mitogen-activated | AAGAATTTTCATCCC |
| | protein kinase kinase 4 | GTATCACAGTGTTTT |
| | (MAP2K4), RefSeqGene on chromosome 17 | TATTGCTCGCCCAGA |
| SEQ ID | GI 654825134 NG_008781 | CTCCTATGACCACTG |
| NO: 503 | Homo sapiens poliovirus receptor | TCTATGTAAACACCC |
| | (PVR), RefSeqGene on chromosome 19 | CACTTTTTCTACACT |
| | | GGTTCTCCTAGGCAC |
| SEQ ID | GI 323276689 NG_028137 | TCATTTTTCAGTGGA |
| NO: 504 | Homo sapiens secretory leukocyte | GGTAGCTGAAGTTCA |
| | peptidase inhibitor | AAGAGGTTCAGTGAT |
| | (SLPI), RefSeqGene on chromosome 20 | TTGCTTCCAACCTGT |
| SEQ ID | GI 187761339 NM_130767 | GTAGATATGTTTAAG |
| NO: 505 | Homo sapiens acyl-CoA thioesterase 12 | TTCCGGGGACCATCT |
| | (ACOT12), mRNA | ACAGTTGGAGATCGT |
| | | CTTGTCTTCACTGCC |
| SEQ ID | GI 1028908926 NG_050619 | CAAAGCAGGTTCCTG |
| NO: 506 | Homo sapiens fatty acid binding protein 7 | ATTCCCAGCTTCCCA |
| | (FABP7), RefSeqGene on chromosome 6 | ATTATGTAGAGATAA |
| | | CTGTTGCCTTAGCAG |
| SEQ ID | GI 221316601 NM_001143935 | GCCTTTACATCTTTT |
| NO: 507 | Homo sapiens carnitine O-octanoyltransferase | GGCAAAAAGCTAACC |
| | (CROT), transcript variant 1, mRNA | AAGAACAAGATGCTT |
| | | CACCCGGATACGTTT |
| SEQ ID | GI 966209818 NM_014031 | CATTTCTCGAGTGAA |
| NO: 508 | Homo sapiens solute carrier family 27 member 6 | TGCAAAAAATCCCTT |
| | (SLC27A6), transcript variant 1, mRNA | CTTTGGCTATGCTGG |
| | | GCCTTATAAGCACAC |
| SEQ ID | GI 166795291 NG_007376 | CATCGTATTCATCCT |
| NO: 509 | Homo sapiens amyloid beta precursor protein | GAGAAAGGGAAACCT |
| | (APP), RefSeqGene on chromosome 21 | AAAGACTTGGGAGAG |
| | | TGTTCTGGGAGGAAA |
| SEQ ID | GI 212549702 NG_008951 | AATGCCCCCCTTAGT |
| NO: 510 | Homo sapiens ATR serine/threonine kinase | TCTGGGAAAGGGGAA |
| | (ATR), RefSeqGene on chromosome 3 | GAAATAGTGTTACTA |
| | | GAACCTGATTGAGAG |
| SEQ ID | GI 224809308 NG_011502 | TAGTTACAGGAAGGT |
| NO: 511 | Homo sapiens cytotoxic T-lymphocyte | AATTTGGCATGCAGC |
| | associated protein 4 | CACTATTTTTGAGTT |
| | (CTLA4), RefSeqGene on chromosome 2 | GATGCAAGCCTCTCT |
| SEQ ID | GI 429535868 NG_033017 | TGGAAACACTGTCTT |
| NO: 512 | Homo sapiens membrane bound | CTCTTGCCGTTGTTT |
| | transcription factor peptidase, site 1 | TCCTCTCTCCAGTGT |
| | (MBTPS1), RefSeqGene on chromosc | TATATCAAGTGAGGA |
| SEQ ID | GI 480355034 KC485577 | AAAAATCAAAACCGC |
| NO: 513 | Homo sapiens bone morphogenenic protein-5 | AATAAATCCAGCTCT |
| | (BMP5) mRNA, complete cds | CATCAGGACTCCTCC |
| | | AGAATGTCCAGTGTT |
| SEQ ID | GI 2909349 Y12863 | GAACAGCAGATCAGG |
| NO: 514 | Homo sapiens mRNA for growth factor FIGF | GCTGCTTCTAGTTTG |
| | | GAGGAACTACTTCGA |
| | | ATTACTCACTCTGAG |
| SEQ ID | GI 727346118 NM_002728 | AAGATGAAACTCCCC |
| NO: 515 | Homo sapiens proteoglycan 2, pro | TTACTTCTGGCTCTT |
| | eosinophil major basic protein | CTATTTGGGGCAGTT |
| | (PRG2), transcript variant 1, mRNA | TCTGCTCTTCATCTA |
| SEQ ID | GI 998614224 NM_001320340 | TGTAAGTGACTTACT |
| NO: 516 | Homo sapiens small VCP interacting protein | TCCAGGGAATCATGC |
| | (SVIP), transcript variant 1, mRNA | CCTATTTCTACCAGC |
| | | AGGTCATACCCAAAT |
| SEQ ID | GI 767172199 NG_042051 | GTGGATGATAAAATC |
| NO: 517 | Homo sapiens kelch like family member 41 | TATGTAGTTGCAGGC |
| | (KLHL41), RefSeqGene on chromosome 2 | AAAGACCTTCAAACA |
| | | GAGGCTTCGCTGGAT |
| SEQ ID | GI 1024846756 NG_047011 | AAGATATTGGGGAGG |
| NO: 518 | Homo sapiens mannose receptor C-type 1 | GGAAGTATTTCGTAG |
| | (MRC1), RefSeqGene on chromosome 10 | TCCTATGATGGGGAC |
| | | TCTGACTTTTAGGGA |
| SEQ ID | GI 224809264 NG_011470 | ACTGTCCCACTACTC |
| NO: 519 | Homo sapiens nitric oxide synthase 2 | TTGGGACAATTTAAT |
| | (NOS2), RefSeqGene on chromosome 17 | GAGATCAGATGTGAA |
| | | GAGCACTCAACCCTT |
| SEQ ID | GI 506949355 NM_199229 | GAATGCTAACACTTA |
| NO: 520 | Homo sapiens ribulose-5-phosphate-3-epimerase | AGCACAGACTAGAGC |
| | (RPE), transcript variant 1, mRNA | TTGCTTGGGTTTCTT |
| | | CCTGCATTACAAGGT |
| SEQ ID | GI 28372408 AY228337 | TTCCAAGGCAACTGC |
| NO: 521 | Homo sapiens TNF receptor-associated factor 6 | CCTTCATACATCAAC |
| | (TRAF6) gene, complete cds | TGAGTGATACATCTC |
| | | TTCCCTCTTGTAGTT |
| SEQ ID | GI 75113052b NG_U4183U | GTCCCTCTTCCTGGA |
| NO: 522 | Homo sapiens DnaJ heat shock protein family | AGCTACTTTCCTAGC |
| | (Hsp40) member C3 | TGTCTGCATGGTGAG |
| | (DNAC3), RefSeqGene on chromosome | TTCCTCATTTTTTTC |
| SEQ ID | GI 970414659 NG_013325 | TGAGATGACAAACTC |
| NO: 523 | Homo sapiens mitogen-activated protein kinase 10 | TATCTAGCCTGGTTT |
| | (MAPK10), RefSeqGene on chromosome 4 | GAGCTCTGTGAATTG |
| | | CTTCAGGAGGTTCTT |
| SEQ ID | GI 340138935 NG_029446 | GGAAATGGAGATTCT |
| NO: 524 | Homo sapiens PYD and CARD domain containing | GGAAGGATAAAGGCC |
| | (PYCARD), RefSeqGene on chromosome 16 | AGTGAGAAGGCATGA |
| | | TAAACATCCATGAGC |
| SEQ ID | GI 167555126 NM_005582 | CTCAGCTAGAACTCC |
| NO: 525 | Homo sapiens CD180 molecule | TCGATTTGGCATTTA |
| | (CD180), mRNA | CCCGCTTACACATTA |
| | | ATGCTCCACAAAGTC |
| SEQ ID | GI 58802450 AY916056 | ACACAACCAAGGGAG |
| NO: 526 | Homo sapiens flavin containing monooxygenase 2 | AAAACTATTCTGTCA |
| | (FMO2) gene, complete cds | AAGAGACGGTGCCAA |
| | | AAGGCAAAAACAAAG |
| SEQ ID | GI 319803083 NG_011853 | GGGTTGAAGAGTAGA |
| NO: 527 | Homo sapiens galactosylceramidase | GGAGGGATTTGAGAG |
| | (GALC), RefSeqGene on chromosome 14 | CTATTGAGGATATCT |
| | | TGGTGACTGAGGGTA |
| SEQ ID | GI 350276269 NG_030323 | TGAGTAGGTGAAACA |
| NO: 528 | Homo sapiens sialic acid | GGAAATACTCAATTT |
| | binding ig like lectin 11 | CCAGATCCTCTTGTG |
| | (SIGLEC11), RefSeqGene on chromosome 19 | CATCCTCCTTGCTCT |
| SEQ ID | GI 574584717 NM_001289177 | TTGCAATAAGTGAAA |
| NO: 529 | Homo sapiens acyl-CoA synthetase | TGCTGCTCTAGGTAG |
| | bubblegum family member 2 | AAGCTCTCCCTGCTG |
| | (ACSBG2), transcript variant 1, mRNA | TTTTTAAGAAGCCAC |
| SEQ ID | GI 189909140 EU779708 | GCAAGTCTAAAGTGA |
| NO: 530 | Homo sapiens protein tyrosine phosphatase-2 | CCCATGTTATGATTC |
| | (PTPN11) mRNA, complete cds | GCTGTCAGGAACTGA |
| | | AATACGACGTTGGTG |
| SEQ ID | GI 406737 X75342 H. sapiens SHB mRNA | ATGACAGAATTTCAG |
| NO: 531 | | AGGCAGGAAAGTGTC |
| | | CGGTCCCAGCATAAA |
| | | GGTATCCAGTTATAT |
| SEQ ID | GI 117606555 EF064752 | ATGCACAGAAGACAT |
| NO: 532 | Homo sapiens CD5 molecule | CCCAGCTCATAAATG |
| | (CD5) gene, complete cds | TACCTTGCAAAGTCT |
| | | TATTTCCCACCCAAT |
| SEQ ID | GI 577019603 NG_033914 | TCAGACTTCTGAGGA |
| NO: 533 | Homo sapiens CD207 molecule | TGGGAGTAAGCCAGA |
| | (CD207), RefSeqGene on chromosome 2 | CAAGGTATCTAGTCA |
| | | GGAATAGGGAAGTTG |
| SEQ ID | GI 430727969 NG_033033 | ACAGCTCTCCCCACC |
| NO: 534 | Homo sapiens ISG15 ubiquitin-like modifier | CTTTGGGAAATTAAC |
| | (ISG15), RefSeqGene on chromosome 1 | TTCTAGGAAAAGAAA |
| | | AAGAGACACAGTTGC |
| SEQ ID | GI 346227162 NG_029971 | CACAAACCTTACAGA |
| NO: 535 | Homo sapiens RELA proto-oncogene, NF-kB subunit | AGGAATAGCAGAGCA |
| | (RELA), RefSeqGene on chromosome 11 | AGATCACATCTCCAC |
| | | CACTGTCCTCAGTTA |
| SEQ ID | GI 353249890 NG_030362 | GAGACAGAAGAATGC |
| NO: 536 | Homo sapiens secreted phosphoprotein 1 | CAGACCCCAGAGGAC |
| | (SPP1), RefSeqGene on chromosome 4 | CTCATGTTATGTAAG |
| | | AAATAGCAATCACCT |
| SEQ ID | GI 257467583 NG_012852 | GCCAATGCCCCTGAG |
| NO: 537 | Homo sapiens signal transducer | ATTAAATGCTGATCC |
| | and activator of transcription 4 | CCATTAGATCTACAC |
| | (STAT4), RefSeqGene on chromosome 2 | ATGTTCAGCTTTCTT |
| SEQ ID | GI 283837929 NG_016244 | CCTCCTCCTCAAAGC |
| NO: 538 | Homo sapiens toll like receptor 5 | CTCACATGAATCTTC |
| | (TLR5), RefSeqGene on chromosome 1 | TTTGAGGTACTCTCT |
| | | GATGCTTCACTCTTC |
| SEQ ID | GI 17059625 AJ420108 | CCCCAGGACTTTATC |
| NO: 539 | Homo sapiens mRNA for F-box only 32 | AACTTGTTCAAGTTC |
| | (FBXO32 gene) | TGAATCCCAGCACAT |
| | | GACAACACTTCAGAA |
| SEQ ID | GI 262331528 NG_013342 | ACTGTTACTGAACAT |
| NO: 540 | Homo sapiens dihydrolipoamide | ACCTTTCAGTTCTCT |
| | S-acetyltransferase | CCTTCTAGGATGCTG |
| | (DLAT), RefSeqGene on chromosome 11 | TTTGGGCTTTCCTGT |
| SEQ ID | GI 190358513 NG_008045 | GTGGTTGCCCCATAT |
| NO: 541 | Homo sapiens dihydrolipoamide dehydrogenase | TCTGCCTTCTGATCC |
| | (DLD), RefSeqGene on chromosome 7 | TTAACCAATAAAACT |
| | | TGGCTTTTGTTTCCC |
| SEQ ID | GI 574997036 NG_033896 | CTGCCTCTCTGAATC |
| NO: 542 | Homo sapiens tubulin beta 4A class IVa | TTTTCCGCAACTCGC |
| | (TUBB4A), RefSeqGene on chromosome 19 | CTCTTACTTCCATTT |
| | | TACAGGTGAGAAAAC |
| SEQ ID | GI 225579089 NG_011672 | CACCTCTTAATTTCC |
| NO: 543 | Homo sapiens decorin | TGCCTGTGTCTTGTG |
| | (DCN), RefSeqGene on chromosome 12 | TGGTTGCATCTACTT |
| | | CAGACCAAAAGACAA |
| SEQ ID | GI 117606581 EF064765 | TGGGATCCAGACTAC |
| NO: 544 | Homo sapiens chemokine | TTTAGCCCATTTAAC |
| | (C-C motif) ligand 21 | CCTTTGAAGTCAAGG |
| | (CCL21) gene, complete cds | AATAAGTCTCCTGGG |
| SEQ ID | GI 1800224 U70065 Human JAK3 gene, complete cds | CCAAATGGTTAAACC |
| NO: 545 | | CTGTCTCCACTGTAA |
| | | ATATGTAACTCAGCT |
| | | GGGCATGGTAATGGG |
| SEQ ID | GI 329665040 NG_028340 | CAAGTAGTGGGGGAC |
| NO: 546 | Homo sapiens granzyme B | TCCTTCACCCACTAG |
| | (GZMB), RefSeqGene on chromosome 14 | ACTGTGATATTTCTC |
| | | TCTGGAAAGAGAAGA |
| SEQ ID | GI 342307060 NG_029601 | TCTTTTACCTGCTGC |
| NO: 547 | Homo sapiens Pim-1 proto-oncogene, | TTCTCCAAAAATCTG |
| | serine/threonine kinase | CCTGGGTTTTGTTCC |
| | (PIM1), RefSeqGene on chromosome 6 | CTATTTTTCTCTCCT |
| SEQ ID | GI 291084831 NG_016755 | TCAGTGGTTTCCTTC |
| NO: 548 | Homo sapiens succinate-CoA ligase alpha subunit | TTGATCTCTCTTATT |
| | (SUCLG1), RefSeqGene on chromosome 2 | TTCTCCAACCACCCC |
| | | CCTTTACTCTTAGGC |
| SEQ ID | GI 1040975354 NM_001737 | CACCAGCCACGTGCA |
| NO: 549 | Homo sapiens complement C9 | CAGCAGTTGCTTTTA |
| | (C9), mRNA | CTAGTCTTAGCTCTA |
| | | CGATTTAAATCCATG |
| SEQ ID | GI 55775471 NM_004271 | AGATGTAATGAAGTC |
| NO: 550 | Homo sapiens lymphocyte antigen 86 | CCCGAATGTATCTGT |
| | (LY86), mRNA | TTCTAAGGAGCCTCT |
| | | TGGCAGTCCTTAAGC |
| SEQ ID | GI 374252456 NM_001252406 | GAATAATCCCCACTC |
| NO: 551 | Homo sapiens zinc finger and BTB domain containing 7B | TGCTCTTAGGATTGA |
| | (ZBTB7B), transcript variant 3, mRNA | ATCCACCCCCATTCT |
| | | GTACATAGCCTCTTC |
| SEQ ID | GI 21655314 AY124010 | TTGGAGGGTCTCTCA |
| NO: 552 | Homo sapiens interleukin 1 receptor, type II | TGGCTGTTACAACTT |
| | (IL1R2) gene, complete cds | TCATGTAGAAGGGCC |
| | | ACACATATAACTCAT |
| SEQ ID | ref\|NG_008776.1\| | ACACATAGTCGAAAT |
| NO: 553 | Homo sapiens ciliary neurotrophic factor | TATAAGTCCCAGGTT |
| | (CNTF), RefSeqGene on | TGTTAGGAAGCTAAA |
| | | GGAAGGAGAGGGGCC |
| SEQ ID | GI 227496937 NG_011931 | GCTGAACAGAGTAGT |
| NO: 554 | Homo sapiens cholinergic receptor | CACCCGCTTGCCATA |
| | nicotinic alpha 4 subunit | TGAGAATCTTTTAAT |
| | (CHRNA4), RefSeqGene on chromosome 20 | TTGTGCCTCAGAAGG |
| SEQ ID | GI 1143077144 NG_052854 | TAAAATGGCCTTCAA |
| NO: 555 | Homo sapiens gem nuclear organelle associated protein 5 | TTGTGGTTGTGCAGC |
| | (GEMIN5), RefSeqGene on chromosome 5 | AGAATCAGCTGGAGA |
| | | GTCTTTGGAAAAATG |
| SEQ ID | GI 207444685 NG_008651 | ATGACTGATTTAGCA |
| NO: 556 | Homo sapiens NLRfamily pyrin domain containing 12 | AAACCATAATTCATC |
| | (NLRP12), RefSeqGene on chromosome 19 | CTCTGAGGCTTGTGA |
| | | AGGGGCAGGATTCAC |
| SEQ ID | GI 260593658 NG_013018 | GTAGCAGACAATTAT |
| NO: 557 | Homo sapiens glycogen phosphorylase, muscle associated | TGATGTTTGCTAGTT |
| | (PYGM), RefSeqGene on chromosome 11 | ATGGAAGGCGGGGCA |
| | | GAATTAGTCACCGTG |
| SEQ ID | GI 350606382 NG_030335 | TGATGAAGTGAGAAA |
| NO: 558 | Homo sapiens SEL1L ERAD E3 ligase adaptor subunit | AGAGAGCCTCAGGAT |
| | (SELIL), RefSeqGene on chromosome 14 | TCATTAGGGTGTTTT |
| | | CAGGCTTACATCCCT |
| SEQ ID | GI 1216866445 NM_005302 | TTTCCCAGTCCAATA |
| NO: 559 | Homo sapiens G protein-coupled receptor 37 | CTTTTCAAAGGGGTT |
| | (GPR37), mRNA | TTTCCCTCCATTGTG |
| | | TGTATTCTGTGGGAT |
| SEQ ID | GI 32891806 AY340485 | CTGGGGGAGACAGGA |
| NO: 560 | Homo sapiens biliverdin reductase B | TTTTGCAATTTTAAA |
| | (flavin reductase | CAGGACAGTCAAGAA |
| | (NADPH)) | AAGCTGCATGGAAAG |
| | (BLVRB) gene, complete cds | |
| SEQ ID | GI 117606545 EF064747 | TCCCTCTTGGAGGAA |
| NO: 561 | Homo sapiens class II, major | AAATAGAAGTAAGTT |
| | histocompatibility complex, transactivator | GTCTCAGGTCAGATT |
| | (CIITA) gene, complete cds | GTCTGGGAAGCCAAG |
| SEQ ID | GI 37654535 AY434089 | ACTCTCATTATTTCC |
| NO: 562 | Homo sapiens insulin-like growth | ATTGCATTTATGTTT |
| | factor binding protein 1 | CCCAGTTCAGCAGCT |
| | (IGFBP1) gene, complete cds | GCAGTTCCCACTGTA |
| SEQ ID | GI 310703611 NG_027845 | GAGTGTCTGTGAACC |
| NO: 563 | Homo sapiens clusterin | AGGTCCGTTCTGTCC |
| | (CLU), RefSeqGene on chromosome 8 | ACTGAGCTGTACTTA |
| | | CGTCTAGATGTATAA |
| SEQ ID | GI 1150763505 NG_052952 | CTGCTAATGTGTATT |
| NO: 564 | Homo sapiens ezrin | TGAGCCTGAAATGCA |
| | (EZR), RefSeqGene on chromosome 6 | CATTGTCCCACCTTT |
| | | CCTCAATGGCAAAAT |
| SEQ ID | GI 293597500 NG_016966 | GGAATTCTGACCCTA |
| NO: 565 | Homo sapiens phosphoglucomutase 1 | AGTATCGTTCATGTT |
| | (PGM1), RefSeqGene on chromosome 1 | TCCTGACCCTTTTCT |
| | | GCCTCCTCATCTCTT |
| SEQ ID | GI 340805863 NG_029510 | AAATAGCTGAGGGTT |
| NO: 566 | Homo sapiens protein tyrosine kinase 2 beta | TAAAGCCACTTAGAC |
| | (PTK2B), RefSeqGene on chromosome 8 | TTCTAGCCAAGTCCT |
| | | GCTCCATCCTTACTG |
| SEQ ID | GI 1007399066 NG_029521 | AGCATACCTGAGACT |
| NO: 567 | Homo sapiens SGT1 homolog, MIS12 | AACCCTATGGTCTAA |
| | kinetochore complex assembly cochaperone | GAAGAACTTACGTTT |
| | (SUGT1), RefSeqGene c | GGAGTCCTGGGCTAA |
| SEQ ID | GI 1021312320 NM_001323565 | CATGATCCCCAGACC |
| NO: 568 | Homo sapiens cysteine dioxygenase type 1 | ACAACAAACCAAAAC |
| | (CD01), transcript variant 1, mRNA | ATAAACTCATGTCTG |
| | | TGTCCCATGGTCATA |
| SEQ ID | GI 572873826 NM_001130080 | ATTCTCAGGAACTCT |
| NO: 569 | Homo sapiens interferon alpha inducible protein 27 | CCTTCTTTGGGTCTG |
| | (IFI27), transcript variant 1, mRNA | GCTGAAGTTGAGGAT |
| | | CTCTTACTCTCTAGG |
| SEQ ID | GI 187423911 NM_003764 | TTGACAGAAGCCAAG |
| NO: 570 | Homo sapiens syntaxin 11 | TAAGGAACTGAAGTT |
| | (STX11), mRNA | GTATCTGACTGTAGG |
| | | GTGAATGTCTGAGGC |
| SEQ ID | GI 1036032362 AH002667 | GGGGTCGCAGATTGT |
| NO: 571 | Homo sapiens cytochrome P450 | GATAGACAGTCTTAC |
| | (CYP1A2) gene, complete cds | ATAGAGTGACATGGG |
| | | GTATAGAGGGGTATT |
| SEQ ID | GI 68248543 DQ096732 | AGTTTCAAATCAGAT |
| NO: 572 | Homo sapiens glutathione peroxidase 7 | GAAGGCGTGGATGCA |
| | (GPX7) gene, complete cds | CTTAGTAAACTGTAG |
| | | AGTGCTTTGCACCTG |
| SEQ ID | GI 262331531 NG_013343 | TTAAAGGACACTTGT |
| NO: 573 | Homo sapiens endothelin receptor type A | TGCTTCTCACGTGCA |
| | (EDNRA), RefSeqGene on chromosome 4 | GAAAACAAGGGCTGC |
| | | TTCTAGATAATCACC |
| SEQ ID | GI 582015216 NG_016002 | GGCTCTAGTGTCACC |
| NO: 574 | Homo sapiens G protein subunit alpha i2 | CTGTTGATTTCCTCT |
| | (GNAI2), RefSeqGene on chromosome 3 | GAAATGGTGAAGTTC |
| | | ATTTCCCTGTTTGTT |
| SEQ ID | GI 289802979 NG_016616 | CCTTACCATAATTCA |
| NO: 575 | Homo sapiens phosphorylase | GAGTCCAGAAGAGCA |
| | kinase catalytic subunit gamma 2 | CCTAAAGGAACTGGA |
| | (PHKG2), RefSeqGene on chromosome 16 | GATGTTGCACACTGA |
| SEQ ID | GI 167860125 NM_002639 | CTAGCAAATTCGGCT |
| NO: 576 | Homo sapiens serpin family B member 5 | TTTGCCGTTGATCTG |
| | (SERPINB5), mRNA | TTCAAACAACTATGT |
| | | GAAAAGGAGCCACTG |
| SEQ ID | GI 1657840 U73304 Human CB1 cannabinoid receptor | CCTCTGAGGATGATG |
| NO: 577 | (CNR1) gene, complete cds | TACTTGCCCTGACCA |
| | | TGCATTTTACCATCA |
| | | CACATGTTCAGAAAG |
| SEQ ID | GI 190570161 NG_008053 | TTGCCATGATACCAT |
| NO: 578 | Homo sapiens acyl-CoA synthetase | CGTGGAAATGAAAGA |
| | long chain family member 4 | CAACGTGAAAATCCT |
| | (ACSL4), RefSeqGene on chromosome X | GCGTCCTTGTGATAT |
| SEQ ID | GI 238018073 NG_012199 | GGTTCAAACAGTGTA |
| NO: 579 | Homo sapiens ketohexokinase | CAGGTTCTAGGGTCC |
| | (KHK), RefSeqGene on chromosome 2 | TGTCTAGCTTTATGA |
| | | ACAGGAGCAAATCCT |
| SEQ ID | GI 402232739 NM_024295 | GACTGTTAGGAGCAG |
| NO: 580 | Homo sapiens derlin 1 | TATTGAGTGGCTGTC |
| | (DERL1), transcript variant 1, mRNA | ACACTTTGAGGCAAC |
| | | TAAAAAGGCTTCAAA |
| SEQ ID | GI 16118554 AF398885 | GAGGTGCTTGAACTC |
| NO: 581 | Homo sapiens ELMOl mRNA, complete cds | GCTTTCTCCATCTTG |
| | | TATGACTCAAACTGC |
| | | CAACTGAACTTCATC |
| SEQ ID | GI 194018739 NG_008192 | CTGGCTCTTCTTAAC |
| NO: 582 | Homo sapiens CD36 molecule | TTTCTCTGCTCTCTC |
| | (CD36), RefSeqGene on chromosome 7 | CTTATTTGGATGGTA |
| | | GGTTTGACACAGGCT |
| SEQ ID | GI 170763484 NG_007481 | CAAATCGAGAGCTGT |
| NO: 583 | Homo sapiens complement C3b/C4b receptor 1 | TCTGCCTCATGACCC |
| | (Knops blood group) | ACTAATGAGAATAAG |
| | (CR1), RefSeqGene on chromosome 1 | TGGGATGTCAGAGAG |
| SEQ ID | GI 207438642 NG_008645 | AGGGAAGATGTTAGT |
| NO: 584 | Homo sapiens dopamine beta-hydroxylase | ATCTGTACCACCCTC |
| | (DBH), RefSeqGene on chromosome 9 | TTGGTTTGATCAGAA |
| | | AGGGCAATGCATGTA |
| SEQ ID | GI 299782605 NG_023187 | AGAGATTGTGTGACT |
| NO: 585 | Homo sapiens major histocompatibility | TGGTTTAAAAAAACA |
| | complex, class 1, B | CGGAACCCAACCATA |
| | (HLA-B), RefSeqGene on chromosome 6 | CGCTCTTTCCAGACA |
| SEQ ID | GI 332164677 NG_028910 | TTTGTGTCTATGCTA |
| NO: 586 | Homo sapiens nuclear receptor subfamily | CCAGAAGGAGGAGAG |
| | 4 group A member 3 | GATGACACTTCCTCT |
| | (NR4A3), RefSeqGene on chromosome 9 | CTGTTTCCCAGATTA |
| SEQ ID | GI 291084846 NG_016762 | GCTCACCTCCCAAGT |
| NO: 587 | Homo sapiens pyruvate dehydrogenase kinase 3 | TTCATGGCTGCAATT |
| | (PDK3), RefSeqGene on chromosome X | CTCTCTGATCTTCAT |
| | | CAAGGATCTTCATTG |
| SEQ ID | GI 117606559 EF064754 | TTATGAAGGATCCAA |
| NO: 588 | Homo sapiens CD40 molecule, TNF | GACTTTCATCTTTGA |
| | receptor superfamily member 5 | ATCCCCTACCCTAAA |
| | (CD40) gene, complete cds | GCCTGGCCTGATCAT |
| SEQ ID | GI 345441766 NG_029866 | AAGTGAGCTCATAAC |
| NO: 589 | Homo sapiens DnaJ heat shock protein family | CTGCCATGGGCAAGT |
| | (Hsp40) member A3 | TTTATATCAGGTAGT |
| | (DNAJA3), RefSeqGene on chromosome | GTACACAGGTGAGAG |
| SEQ ID | GI 110349787 NM_018489 | TTAAAATGGGAGGTG |
| NO: 590 | Homo sapiens ASH1 like histone | CTTCTGATGTATCTA |
| | lysine methyltransferase | CCGCTAAATCCCCAT |
| | (ASHIL), mRNA | TCAGTGCAGTAGGAG |
| SEQ ID | GI 95147553 NM_003956 | TGGATGGTGTTGACT |
| NO: 591 | Homo sapiens cholesterol 25-hydroxylase | TCTGACTCTAAAAGC |
| | (CH25H), mRNA | AATCAAACATGTTTC |
| | | TGCTGGACAGTGACC |
| SEQ ID | GI 60391308 D50017 | ACCTCTGCTGGACTC |
| NO: 592 | Homo sapiens PDGFRA gene for alpha- | ATGAATTCAGGTATT |
| | platelet-derived growth factor receptor, | TCTGGGAGGTTCTGG |
| | complete cds | GTTACTCTAGAGTAG |
| SEQ ID | GI 974987497 NG_011800 | TGTCCTGTATGTGGG |
| NO: 593 | Homo sapiens ceruloplasmin | AGTAGTGACAAAACA |
| | (CP), RefSeqGene on chromosome 3 | GCACATGGTGTGATA |
| | | GAATGAGATGGACTG |
| SEQ ID | GI 1150561155 NG_052944 | AATGCAGCTGAGAGA |
| NO: 594 | Homo sapiens TNF superfamily member 12 | TCACATGAAATGAGA |
| | (TNFSF12), RefSeqGene on chromosome 17 | ACAGAGAAATGATGG |
| | | CTGGGTTTGGTAACG |
| SEQ ID | GI 927669118 NM_001166693 | GGTTGATTCAATGCC |
| NO: 595 | Homo sapiens AF4/FMR2 family member 1 | TTGGGAACTATTTTT |
| | (AFF1), transcript variant 1, mRNA | GCACATTGGAAGCCT |
| | | CAAAAACAGTCCAGA |
| SEQ ID | GI 29542 X06596 Human mRNA for | GAATGGTAAAGTTGA |
| NO: 596 | complement component Cls | AGACCCAGAGAGCAC |
| | | TTTGTTTGGTTCTGT |
| | | CATCCGCTACACTTG |
| SEQ ID | GI 728797213 LN680437 | TTACACCTTTGATGG |
| NO: 597 | Homo sapiens mRNA for CD47 | AGCTCTAAACAAGTC |
| | | CACTGTCCCCACTGA |
| | | CTTTAGTAGTGCAAA |
| SEQ ID | GI 20147702 AF493905 | AATTATTGCTATCAT |
| NO: 598 | Homo sapiens guanine nucleotide | TAGGGCTATGGGGAG |
| | binding protein alpha il | GTTGAAGATAGACTT |
| | (GNAIl) mRNA, complete cds | TGGTGACTCAGCCCG |
| SEQ ID | GI 169790829 NM_001754 | GTCCAGTTCTTCCCC |
| NO: 599 | Homo sapiens runt related transcription factor 1 | CTTCACATAAAGGAA |
| | (RUNX1), transcript variant 1, mRNA | CATGGTTTGTCAGCC |
| | | TTTCTCCTGTTTATG |
| SEQ ID | GI 209977031 NG_008839 | CCCCAAAGCCTCTTC |
| NO: 600 | Homo sapiens 24-dehydrocholesterol reductase | TTCTTCAGGGAAAAC |
| | (DHCR24), RefSeqGene on chromosome 1 | ACCTTCAGTTCTTTT |
| | | AACTATTCCCATGGG |
| SEQ ID | GI 238028972 NG_011992 | AACTGGGACCTAGGA |
| NO: 601 | Homo sapiens nitric oxide synthase 3 | AAATGAGTCATCCTT |
| | (NOS3), RefSeqGene on chromosome 7 | GGTCATGCACATTTC |
| | | AAATGGTGGCTTAAT |
| SEQ ID | GI 545687789 NM_015901 | CAGACAGAAATCCAG |
| NO: 602 | Homo sapiens nudix hydrolase 13 | GTGAACTTGAGAGAA |
| | (NUDT13), transcript variant 1, mRNA | TTAGAGACAGCTGCC |
| | | TGGTTCAGTCATGAT |
| SEQ ID | GI 20303097 AF503166 | CAAAAGTGCAATGGA |
| NO: 603 | Homo sapiens xeroderma pigmentosum, | TTGCTTCTCATGAGC |
| | complementation group A | TTCCCGTTACTAGAA |
| | (XPA) gene, complete cds | GCGCTCAAATAGGAA |
| SEQ ID | GI 224809341 NG_011508 | CTGAATGAAAAGTGC |
| NO: 604 | Homo sapiens insulin like growth | TCCTAAAATCTTTCC |
| | factor binding protein 3 | TGTCCCCTTTTAAGA |
| | (IGFBP3), RefSeqGene on chromosome 7 | CTTCACTGGGCAGGG |
| SEQ ID | GI 1069415403 NG_032899 | AACTCCCATGTGCTA |
| NO: 605 | Homo sapiens protein C receptor | GGTAATGTATGAGGT |
| | (PROCR), RefSeqGene on chromosome 20 | GCCAGAGTTACCCAA |
| | | AAAACAAACAAAGCA |
| SEQ ID | GI 169790828 NM_002755 | CTTTCTTACCCAGAA |
| NO: 606 | Homo sapiens mitogen-activated | GCAGAAGGTGGGAGA |
| | protein kinase kinase 1 | ACTGAAGGATGACGA |
| | (MAP2K1), mRNA | CTTTGAGAAGATCAG |
| SEQ ID | GI 117606541 EF064745 | CTTCTAAGACCCTGG |
| NO: 607 | Homo sapiens chemokine | AAGACTCTTAGAAGT |
| | (C-X3-C motif) ligand 1 | CCTTGAGGTCCAGCC |
| | (CX3CL1) gene, complete cds | TAAAGGAGAATTCCT |
| SEQ ID | GI 1143077143 NG_052860 | CATCCTCGAGGTTTT |
| NO: 608 | Homo sapiens adiponectin receptor 1 | CATAGGCTTCCAGCT |
| | (ADIPOR1), RefSeqGene on chromosome 1 | TCCTGTTCTCCTTAG |
| | | CTCAATTGTTCACTA |
| SEQ ID | GI 347658933 NG_030027 | GTAAAGAGCAACACA |
| NO: 609 | Homo sapiens AT-rich interaction domain 5B | ATTCTTTGTAAAGCC |
| | (ARID5B), RefSeqGene on chromosome 10 | ATTGGCCCCACAGAA |
| | | AGCTCCATGGAAAAT |
| SEQ ID | GI 226492969 NG_011817 | TAGGAATTAGAGCCC |
| NO: 610 | Homo sapiens LIF receptor alpha | TCTCTGCAATATCAT |
| | (LIFR), RefSeqGene on chromosome 5 | CGTTGTTTTCCTGGC |
| | | ACAATGGGAGTGTAT |
| SEQ ID | GI 237858735 NG_012106 | CTTTATCTGTGGAAT |
| NO: 611 | Homo sapiens 8-oxoguanine DNA glycosylase | GGGATGGTAACTCAG |
| | (OGG1), RefSeqGene on chromosome 3 | CCCTCACAAGATGCT |
| | | ATGAGGAATGAGAGG |
| SEQ ID | GI 686223163 NG_034261 | CAGAGTTAGTGGTAC |
| NO: 612 | Homo sapiens primase | AGACTCTAGGGTTGT |
| | (DNA) subunit 2 | GGTTTCTTCACGGAA |
| | (PRIM2), RefSeqGene on chromosome 6 | CCTGTTTTAAGGCAT |
| SEQ ID | GI 305410826 NG_027551 | TGCCATCAAGGAGGC |
| NO: 613 | Homo sapiens transient receptor potential | CATCACCAGAAATTG |
| | cation channel subfamily M member 4 | ATTTTCTTCATGCTA |
| | (TRPM4), RefSeqGene or | CACATACTTCCATCC |
| SEQ ID | GI 307775406 NM_015364 | TCAACACCATGAATC |
| NO: 614 | Homo sapiens lymphocyte antigen 96 | TTCCAAAGCGCAAAG |
| | (LY96), transcript variant 1, mRNA | AAGTTATTTGCCGAG |
| | | GATCTGATGACGATT |
| SEQ ID | GI 375151559 NM_005651 | CAACATACCTGATTC |
| NO: 615 | Homo sapiens tryptophan 2,3-dioxygenase | CCCGACACTGGATAC |
| | (TD02), mRNA | CGAAGATGAACCCAA |
| | | CCATTCACAAATTTC |
| SEQ ID | GI 117606567 EF064758 | CCTGCCAAATATTCA |
| NO: 616 | Homo sapiens chemokine | TATACCTGTCCACTT |
| | (C-C motif) receptor 7 | GGGGTCATGAGGAGA |
| | (CCR7) gene, complete cds | TGCAAATGAAACGTG |
| SEQ ID | GI 410991940 NG_032977 | TTCAAGCTAGAACTC |
| NO: 617 | Homo sapiens interleukin 12 receptor subunit beta 2 | AAAACTCCATACTTG |
| | (IL12RB2), RefSeqGene on chromosome 1 | CCAGGTACACATGGT |
| | | CCTGTTTACCCATCT |
| SEQ ID | GI 238776809 NG_012246 | GATATTGGGACTGAG |
| NO: 618 | Homo sapiens N-acetyltransferase 2 | AGATAGGAGGCCAGG |
| | (NAT2), RefSeqGene on chromosome 8 | AGAAGGCCAAAGAAA |
| | | AACTGACTTCTGAAG |
| SEQ ID | GI 194018405 NG_008205 | ATGAAAGGATCGGAT |
| NO: 619 | Homo sapiens phosphoenolpyruvate carboxykinase 1 | GAAGGTCGTTGGTGA |
| | (PCK1), RefSeqGene on chromosome 20 | CTCACTGCTCTGACG |
| | | TTTTACTTGAACTAG |
| SEQ ID | GI 359279909 NM_020663 | GTCAAAGGCCATCTC |
| NO: 620 | Homo sapiens ras homolog family member J | ACATTTTACAAATCC |
| | (RHOJ), mRNA | CCAGCTCATGAACGT |
| | | GAAGCTGATAGGAAA |
| SEQ ID | GI 42519913 NM_012448 | GAGATTTTTGATGAA |
| NO: 621 | Homo sapiens signal transducer and | AGGTGTGCTCGCTCT |
| | activator of transcription 5B | CTGCGTTCTATCTTC |
| | (STAT5B), mRNA | TCTCTCCTCCTTGTT |
| SEQ ID | GI 48255965 NM_006759 | CTCTGACAATGAGTG |
| NO: 622 | Homo sapiens UDP-glucose pyrophosphorylase 2 | AAAAGCGGGAATTTC |
| | (UGP2), transcript variant 1, mRNA | CTACAGTGCCCTTGG |
| | | TTAAATTAGGCAGTT |
| SEQ ID | GI 117606505 EF064727 | TGGAGGAGAAGTGGA |
| NO: 623 | Homo sapiens inducible T-cell co-stimulator | TAGATTCTGTTGGAC |
| | (ICOS) gene, complete cds | ATAAAACAGAGGTGC |
| | | TGGAAATTAGGCGTT |
| SEQ ID | GI 269914179 NG_015979 | TCTCTCCTTTCATGG |
| NO: 624 | Homo sapiens nitric oxide synthase 1 adaptor protein | AATCTCCCTGTGCAA |
| | (NOSIAP), RefSeqGene on chromosome 1 | TAAAGGAAAGCTGAG |
| | | GACATTCTCCCATAC |
| SEQ ID | GI 557878734 NM_001995 | AAGGCATCACATTGC |
| NO: 625 | Homo sapiens acyl-CoA synthetase long chain family member 1 | ACCCTGAATTATTTT |
| | (ACSL1), transcript variant 1, mRNA | CTATCGACAATGGCC |
| | | TTCTGACTCCAACAA |
| SEQ ID | GI 68215223 NM_003965 | GTAGTCTTCCTTCTG |
| NO: 626 | Homo sapiens C-C motif chemokine receptor like 2 | ATGTGGGCGCCCTAC |
| | (CCRL2), transcript variant 1, mRNA | AATATTGCATTTTTC |
| | | CTGTCCACTTTCAAA |
| SEQ ID | GI 735367774 NM_000211 | AGGACTCCAGCAATG |
| NO: 627 | Homo sapiens integrin subunit beta 2 | TGGTCCATCTCATTA |
| | (ITGB2), transcript variant 1, mRNA | AGAATGCTTACAATA |
| | | AACTCTCCTCCAGGG |
| SEQ ID | GI 117606359 NM_015310 | GTACTGGACCAAGAT |
| NO: 628 | Homo sapiens pleckstrin and Sec7 domain containing 3 | GCTGTTTCTAGTTTT |
| | (PSD3), transcript variant 1, mRNA | TCAGTTCAGCAGGTG |
| | | GAAAAAGAGCTGGAC |
| SEQ ID | GI 960513892 NM_001317781 | AAAGAGCAAAGTCAG |
| NO: 629 | Homo sapiens sorting nexin 25 | GAAACAAAACAGAGA |
| | (SNX25), mRNA | GCACAGCAAAAGCTG |
| | | CTTGAAAACATTCCA |
| SEQ ID | GI 27979118 AY219866 | TCCTTATGCCTCAGA |
| NO: 630 | Homo sapiens caspase 3, apoptosis-related cysteine protease | GTGTCATTGGGAGGA |
| | (CASP3) gene, complete cds | CTCACGAAAATACGC |
| | | TTGTACTTTGAGTTC |
| SEQ ID | GI 32187528 AY324876 | GCAATAGCCCACAAT |
| NO: 631 | Homo sapiens lactoperoxidase | TCCTAAAGTCCTCAG |
| | (LPO) gene, complete cds | CCTGTACATCACCTG |
| | | AAAGAGGACATCTAA |
| SEQ ID | GI 263191400 NG_015828 | CAATCTGCACAGATG |
| NO: 632 | Homo sapiens glutamic-pyruvic transaminase | CTCTTCTGAAGTAGG |
| | (GPT), RefSeqGene on chromosome 8 | CATGATAGTCCCCAT |
| | | TTGATAGACGTGGAA |
| SEQ ID | GI 1015809840 NG_009004 | CACATTCCCCTTCTT |
| NO: 633 | Homo sapiens potassium voltage- | CCCACAGCCTCCTGT |
| | gated channel subfamily Q member 2 | GTAGGAAATCTTATG |
| | (KCNQ2), RefSeqGene on chromo [symbol] | GAAATTTCCCAAAAG |
| SEQ ID | GI 430768625 NG_033035 | AGATGTAGACTGGGG |
| NO: 634 | Homo sapiens kelch like family member 40 | AGGTTGACTGATAGC |
| | (KLHL40), RefSeqGene on chromosome 3 | CACCTACTTGAAGGT |
| | | TGAAGACAGTCAATA |
| SEQ ID | GI 1008909437 NG_023257 | CTGCATTCTATGTGC |
| NO: 635 | Homo sapiens lactotransferrin | AGGATGAAGAAAGAG |
| | (LTF), RefSeqGene on chromosome 3 | AAGGAGAAGAAAGGG |
| | | CAAAAGGCATGTACC |
| SEQ ID | GI 318064850 NG_028074 | CTCATGGGTCAATAC |
| NO: 636 | Homo sapiens ras related dexamethasone induced 1 | TACTGTTATTGTTGC |
| | (RASD1), RefSeqGene on chromosome 17 | TATTGTTATTACCAG |
| | | GCCCCGCCCTCCTAA |
| SEQ ID | GI 1030311320 NG_050664 | GGGCCTAAGAAGGAT |
| NO: 637 | Homo sapiens sirtuin 1 | GTGACATGTCCTATG |
| | (SIRT1), RefSeqGene on chromosome 10 | ATAAAGGACCAGGAG |
| | | TTTGGGATTTGAGAG |
| SEQ ID | GI 187761339 NM_130767 | TCAGTATCAAGGTCA |
| NO: 638 | Homo sapiens acyl-CoA thioesterase 12 | TGGTACAGGATATGC |
| | (ACOT12), mRNA | TCACTGGCATTGAGA |
| | | AGCTTGTTAGTGTGG |
| SEQ ID | GI 33413399 NM_001984 | GCAAAAAGCAATCAG |
| NO: 639 | Homo sapiens esterase D | CAATTGGACAGGAAA |
| | (ESD), mRNA | AGAATGGCATTGAAG |
| | | CAGATTTCCAGCAAC |
| SEQ ID | GI 42416963 AY534685 | GCTGGAGATTCTCAG |
| NO: 640 | Homo sapiens insulin-like growth | CCAGGGACTTGTGCA |
| | factor binding protein 5 | TCAGTGTGTATTTCC |
| | (IGFBP5) gene, complete cds | TATGTGTACAGTTAT |
| SEQ ID | GI 77744394 DQ230990 | TGCCAAGAGGAATGT |
| NO: 641 | Homo sapiens peroxiredoxin 6 | TAAGTTGATTGCCCT |
| | (PRDX6) gene, complete cds | TTCAATAGACAGTGT |
| | | TGAGGACCATCTTGC |
| SEQ ID | GI 224809245 NG_011450 | GTAGTTTGGGAACCC |
| NO: 642 | Homo sapiens interferon regulatory factor 1 | CTGACATATGACAAT |
| | (IRF1), RefSeqGene on chromosome 5 | GCCCCCTTAGATAAG |
| | | CTCAAGTTACAGGAA |
| SEQ ID | GI 584613557 NG_033959 | TTGTTTGAAGTGGAG |
| NO: 643 | Homo sapiens poly | AAGGATGGTGAGAAA |
| | (ADP-ribose) polymerase 2 | GAAGCCTTCAGAGAG |
| | (PARP2), RefSeqGene on chromosome 14 | GACCTTCATAACAGG |
| SEQ ID | GI 77702091 DQ217939 | GAAGACAGAAGGGCA |
| NO: 644 | Homo sapiens mannose-binding protein C | GTTTGTGGATCTGAC |
| | (MBL2) gene, complete cds | AGGAAATAGACTGAC |
| | | CTACACAAACTGGAA |
| SEQ ID | GI 225735572 NG_011717 | CCACAGTGATGGTTA |
| NO: 645 | Homo sapiens alpha-2-macroglobulin | AGAACGAGGACAGTC |
| | (A2M), RefSeqGene on chromosome 12 | TGGTCTTTGTCCAGA |
| | | CAGACAAATCAATCT |
| SEQ ID | GI 335334987 NG_029076 | ACAGTGGGAAGGCTG |
| NO: 646 | Homo sapiens C-X-C motif chemokine receptor 3 | AAAGTAAGACTGAAA |
| | (CXCR3), RefSeqGene on chromosome X | GGTAATTTGGGATCA |
| | | GGTGATGGTATTGGA |
| SEQ ID | ref\|NM_004420.2\|Homo sapiens dual | CTTCTGCTCCCGGCC |
| NO: 647 | specificity phosphatase 8 | CCTCCCTTTGTATTT |
| | (DUSP8), mRNA | GGAGACAATGTGTTG |
| | | TAATAAAGCTTAAAG |
| SEQ ID | GI 1042161084 NG_046982 | GGAAGAGAATGGATG |
| NO: 648 | Homo sapiens ficolin 1 | TTAAAAAATGCAAGG |
| | (FCN1), RefSeqGene on chromosome 9 | ATAGCCATTGGGTAG |
| | | AACGCCATACAGCCA |
| SEQ ID | GI 654825055 NG_028206 | TTTGGTGGAGAAGTG |
| NO: 649 | Homo sapiens prostaglandin-endoperoxide synthase 2 | GGTTTTCAAATCATC |
| | (PTGS2), RefSeqGene on chromosome 1 | AACACTGCCTCAATT |
| | | CAGTCTCTCATCTGC |
| SEQ ID | GI 341572552 NG_029517 | TAGTGCCATCAATGC |
| NO: 650 | Homo sapiens retinoid X receptor gamma | ACACAGGGACTTTTG |
| | (RXRG), RefSeqGene on chromosome 1 | GAGTCATTGTAGAGT |
| | | TATGCACTGTACAGA |
| SEQ ID | GI 325910849 NG_028199 | TGTGGCATGTTATCT |
| NO: 651 | Homo sapiens scavenger receptor class B member 1 | AAAGAGTAAAGGTAC |
| | (SCARB1), RefSeqGene on chromosome 12 | CATGCCTTTGCCTTT |
| | | CTGGGCCTTGAAATC |
| SEQ ID | GI 955654849 NM_001928 | AGCAATGAAGTCATC |
| NO: 652 | Homo sapiens complement factor D | CACTCCTGCATCTGG |
| | (CFD), transcript variant 1, mRNA | TTGGTCTTTATTGAG |
| | | CACCTACTATATGCA |
| SEQ ID | GI 50345287 AY682848 | ACCTTCTAATTATAG |
| NO: 653 | Homo sapiens receptor | CTTGGTCTTTCTGGT |
| | (TNFRSF)-interacting serine-threonine kinase 1 | TGCTAGCCCCCATTC |
| | (RIPK1) gene, complete cds | TGAAGTTGCCTAGTT |
| SEQ ID | GI 30039656 AY273786 | GAGAACCCAGGTCGT |
| NO: 654 | Homo sapiens tumor necrosis factor, | CTTTCTATTTTCAGG |
| | alpha-induced protein 2 | TCAGCTGATTAGCCA |
| | (TNFAIP2) gene, complete cds | CCTTAGTTCCATCTG |
| SEQ ID | GI 340805863 NG_029510 | TCTGTTTTTCCTGTC |
| NO: 655 | Homo sapiens protein tyrosine kinase 2 beta | TGATCCAGGAGCACA |
| | (PTK2B), RefSeqGene on chromosome 8 | CATTGCACTTAGCTG |
| | | TCATATCCCTTTAGT |
| SEQ ID | GI 506949355 NM_199229 | AAAGGATCTGGGAAG |
| NO: 656 | Homo sapiens ribulose-5-phosphate-3-epimerase | TGGTAGAATTTCTGG |
| | (RPE), transcript variant 1, mRNA | TCTGTACTTTTACAA |
| | | ATGGAGCCCTTGGGA |
| SEQ ID | GI 632794850 NM_002957 | GCCTGTCACTGCTCT |
| NO: 657 | Homo sapiens retinoid X receptor alpha | GCCTAAGAGATGTGT |
| | (RXRA), transcript variant 1, mRNA | TGTCACCCTCCTTAT |
| | | TTCTGTTACTACTTG |
| SEQ ID | GI 209447071 NM_005639 | GGCTGTTTCCCAGTA |
| NO: 658 | Homo sapiens synaptotagmin 1 | AAACTGGAATTCCTG |
| | (SYT1), transcript variant 1, mRNA | TCGTTACTGTTTCCT |
| | | TATCAAAGAAGGGGC |
| SEQ ID | GI 21397157 AF517525 | ACTGTATGAGGTAAG |
| NO: 659 | Homo sapiens cyclin D3 | GAGGTGGAAGTCAAT |
| | (CCND3) gene, complete cds | GGTCTAAAGCAGGGA |
| | | TCATTGAGCCTTTTC |
| SEQ ID | GI 94717615 DQ523828 | AGACATTCTGCTGGA |
| NO: 660 | Homo sapiens transglutaminase 2 | AGCTTGCACATATTA |
| | (C polypeptide, protein-glutamine- | GCTCAGTTAATCCTC |
| | gamma-glutamyltransferase) | ACAAGAACCCTCAGT |
| | (TGM2) ge | |
| SEQ ID | GI 237858769 NG_012123 | TCCATTTCTACTAAC |
| NO: 661 | Homo sapiens C-C motif chemokine ligand 2 | TGGAGTTGCAGAGGG |
| | (CCL2), RefSeqGene on chromosome 17 | AGAGAAGGGATTCTG |
| | | CTTCTACATTCCTGA |
| SEQ ID | GI 1078569903 NG_051800 | TGTTGCAGATCACAG |
| NO: 662 | Homo sapiens dedicator of cytokinesis 2 | GAAATCATGACCTAG |
| | (DOCK2), RefSeqGene on chromosome 5 | GCCATCAATGTTAGA |
| | | ATTCCAATTGCCTGG |
| SEQ ID | GI 237858739 NG_012110 | ATCGTGTGTTGGGGA |
| NO: 663 | Homo sapiens programmed cell death 1 | TTGTGTGTATATTGC |
| | (PDCD1), RefSeqGene on chromosome 2 | AGATTTTGTATGTGT |
| | | GTTGGGGATTGTGGT |
| SEQ ID | GI 647570744 NG_034141 | CCGGACTCTCCAAAG |
| NO: 664 | Homo sapiens STIP1 homology and | ATTTGGAAAACTTTA |
| | U-box containing protein 1 | CAAAACCAAGTGGAA |
| | (STUB1), RefSeqGene on chromosome 16 | TCAAGCGGATAGGCT |
| SEQ ID | GI 14594701 AY037115 | CTTACAGATCTCTTA |
| NO: 665 | Homo sapiens thymic stromal lymphopoietin | CACTCGTGGTGGGAA |
| | (TSLP) mRNA, complete cds | GAGTTTAGTGTGAAA |
| | | CTGGGGTGGAATTGG |
| SEQ ID | GI 94717638 DQ496106 | ACCAATTGCATTTTC |
| NO: 666 | Homo sapiens aconitase 1, soluble | TGTTTGCTGCTTAAC |
| | (ACO1) gene, complete cds | AGCTTCCATGGGAGA |
| | | AACGATCAACATGTG |
| SEQ ID | GI 325053737 NG_028166 | CAGATCCAGCCTGAT |
| NO: 667 | Homo sapiens aminoacyl tRNA synthetase | CTTCACACTAATGAT |
| | complex interacting multifunctional protein 1 | GAGTGTGTGGCTACA |
| | (AIMP1), RefSeqG | TACAAAGGAGTTCCC |
| SEQ ID | GI 306518675 NG_027688 | CTCAAAACATCAATG |
| NO: 668 | Homo sapiens CD4 molecule | CCTACCAGAGCCTGC |
| | (CD4), RefSeqGene on chromosome 12 | CAGGTAATGTAAATG |
| | | AGAAAAGCAGACTGG |
| SEQ ID | GI 345091009 NG_029843 | TATACTAAAGAGGTC |
| NO: 669 | Homo sapiens nuclear receptor | CTGTTGCCCCAAGCC |
| | subfamily 1 group H member 4 | CAATGAAAGAGTCTT |
| | (NR1H4), RefSeqGene on chromosome 12 | CAAGACGATCACTTG |
| SEQ ID | GI 1024336776 NG_029865 | CAGAACTGCAGATCC |
| NO: 670 | Homo sapiens protein inhibitor of activated STAT 2 | AGACTCCACGCTTGC |
| | (PIAS2), RefSeqGene on chromosome 18 | TTTGTGCTTAATTGT |
| | | ACATCTCATTCACAC |
| SEQ ID | GI 31657141 NM_181501 | GCAGCTTTTGAATGC |
| NO: 671 | Homo sapiens integrin subunit alpha 1 | TAAGGGTTCCAGTAT |
| | (ITGA1), mRNA | GTGACCCAAAGAAAG |
| | | AGTTGTCTCAACTCC |
| SEQ ID | GI 111161293 NM_005746 | TATCTTTACATAGGA |
| NO: 672 | Homo sapiens nicotinamide phosphoribosyltransferase | CGCCAGCAGGGAATT |
| | (NAMPT), mRNA | TTGTTACACTGGAGG |
| | | AAGGAAAAGGAGACC |
| SEQ ID | GI 902763272 NM_002674 | ACATTCAGGTTGGGG |
| NO: 673 | Homo sapiens pro-melanin concentrating hormone | AAAGGCTTTCAGAAG |
| | (PMCH), mRNA | GAAGACACTGCAGAA |
| | | AAATCAGTTATTGCT |
| SEQ ID | GI 41349444 NM_014011 | TACCCATGTTAGCAG |
| NO: 674 | Homo sapiens suppressor of cytokine signaling 5 | ACAGGGAGCTTGGAA |
| | (SOCS5), transcript variant 1, mRNA | AGTCCACACACAGAT |
| | | TGATTACATACACTG |
| SEQ ID | GI 32263626 AY325910 Homo sapiens | TTACTAGCTGGGTGA |
| NO: 675 | hydroxysteroid (17-beta) dehydrogenase 2 | CCTGGGTCAGGTCAC |
| | (HSD17B2) gene, complete cds | CTTACTTGTCATGTC |
| | | TCTATCTTCAAATGA |
| SEQ ID | GI 261599023 NG_013217 Homo sapiens | AGTGTTTGGAGGCTG |
| NO: 676 | apoptosis inducing factor mitochondria | AGGGTACAGAATAGA |
| | associated 1 (AIFM1), RefSeqGene on chromosome [symbol] | AATGTGTGAACTGCT |
| | | TGATTCAGAAGACCA |
| SEQ ID | GI 291045455 NG_016708 Homo sapiens MDM2 | AGGTTATGTAAAAAG |
| NO: 677 | proto-oncogene (MDM2), RefSeqGene on | GCTGGGGATGTGGGA |
| | chromosome 12 | GAGTTTGTTATCCAA |
| | | ATTGCATGGTGGAAA |
| SEQ ID | GI 224809262 NG_011468 Homo sapiens matrix | TGAGGAAACACTTCA |
| NO: 678 | metallopeptidase 9 (MMP9), RefSeqGene on | AAGTGGTAAGACATT |
| | chromosome 20 | TGCCCGAGGTCCTGA |
| | | AGGAAGAGAGTAAAG |
| SEQ ID | GI 1159261282 NG_052987 Homo sapiens zinc | AGTTGGTCTCCTTCC |
| NO: 679 | finger protein 148 (ZNF148), RefSeqGene | TTTCTCTTTCCTGCT |
| | on chromosome 3 | CCGTTGTAGTTTTCA |
| | | TCATTCTCTGCTCTG |
| SEQ ID | GI 539847481 NM_003837 Homo sapiens | CCATCGGAACCATCT |
| NO: 680 | fructose-bisphosphatase 2 (FBP2), mRNA | TTGCCATCTATAGAA |
| | | AGACCTCAGAGGATG |
| | | AGCCTTCTGAAAAGG |
| SEQ ID | GI 219842351 NM_002522 Homo sapiens | GTGAAAAGCAACATG |
| NO: 681 | neuronal pentraxin 1 (NPTX1), mRNA | GACATTTCCTTCTAG |
| | | TCCTTCCAAGGAGGG |
| | | GAGAGAAGTGTATGT |
| SEQ ID | GI 394582046 NM_003877 Homo sapiens | TAAAGTTATGGGCTG |
| NO: 682 | suppressor of cytokine signaling 2 | TACCTGCCCCCTTTG |
| | (SOCS2), transcript variant 1, mRNA | CAATTTGGAAAGCAT |
| | | GGTTTAGAAACTACA |
| SEQ ID | GI 746815979 NM_032525 Homo sapiens | GCTGCAGCTGGAGAG |
| NO: 683 | tubulin beta 6 class V (TUBB6), | AATCAACGTCTACTA |
| | transcript variant 1, mRNA | CAATGAGTCATCGTC |
| | | TCAGAAATATGTGCC |
| SEQ ID | GI 47419892 AY623660 Homo sapiens TNF | TGTAAATTATGCAAC |
| NO: 684 | receptor-associated factor 2 (TRAF2) | TTCAAAGCCTGCCCT |
| | gene, complete cds | GTTTTGCAGTGTATT |
| | | CCATCCTTGAGGAGT |
| SEQ ID | GI 342307072 NG_029607 Homo sapiens | GGTTTTTTGAGGGGG |
| NO: 685 | heat shock transcription factor 2 | AGCTCCCGATTGCTC |
| | (HSF2), RefSeqGene on chromosome 6 | CTGCTTAGAAGGCCC |
| | | TTGAACTAAAAATAG |
| SEQ ID | GI 312261284 NM_016441 Homo sapiens | ATTGCAGAACCAGAT |
| NO: 686 | cysteine rich transmembrane BMP regulator | GCAAGATTCAGTGGC |
| | 1 (CRIM1), mRNA | TTCTACAGCATGCAA |
| | | AAACAGAACCATCTA |
| SEQ ID | GI 332000013 NM_002733 Homo sapiens | TTCATGTCCAAGTCT |
| NO: 687 | protein kinase AMP-activated non- | CTGGAAGAGCTACAG |
| | catalytic subunit gamma l (PRKAG1), | ATTGGCACCTATGCC |
| | transcript variant 1 | AATATTGCTATGGTT |
| SEQ ID | GI 196259787 NM_001098531 Homo sapiens | GTCAGTGAACAGTTC |
| NO: 688 | Rap guanine nucleotide exchange | TCTTCCTTCATTTCT |
| | factor 3 (RAPGEF3), transcript | CCACCTGTAAGCCAG |
| | variant 1, mRNA | GTATGATAAACGCCA |
| SEQ ID | GI 18653445 AF479264 Homo sapiens ligase | GGTATGTAACCAGAC |
| NO: 689 | IV, DNA, ATP-dependent (LIG4) gene, | CATAGCTTTCCCTAG |
| | complete cds | TGTGCTTATCCAGGA |
| | | GTCAACTCTAGGTGT |
| SEQ ID | GI 1024336776 NG_029865 Homo sapiens | GCTGAAGTTGCGTTT |
| NO: 690 | protein inhibitor of activated STAT 2 | AGGAATCTCTCCTCT |
| | (PIAS2), RefSeqGene on chromosome 18 | TCTTATGGCTGATGC |
| | | TACCGTTTAGTCAGG |
| SEQ ID | GI 1000232109 NG_046737 Homo sapiens | TGAGGAAGCTGAGGA |
| NO: 691 | protein phosphatase 1 regulatory subunit | TGATGGTTTTGATAG |
| | 15B (PPP1R15B), RefSeqGene on chromosome 1 | TGATAGCTCACTGTC |
| | | AGACTCAGACCTTGA |
| SEQ ID | GI 384551644 NM_005428 Homo sapiens vav | GCATTTCTTGAAGCC |
| NO: 692 | guanine nucleotide exchange factor 1 | CCTGCAACGGTTCCT |
| | (VAV1), transcript variant 1, mRNA | GAAACCTCAAGACAT |
| | | TGAGATCATCTTTAT |
| SEQ ID | GI 1187633042 KY500729 Homo sapiens | TCCAAGATCTTAAAG |
| NO: 693 | isolate MCF UBD gene, complete cds | CCACGGAGAAGCCTC |
| | | TCATCTTATGGCATT |
| | | GACAAAGAGAAGACC |
| SEQ ID | GI 1078569903 NG_051800 Homo sapiens | TCTTGGTGAGATAGG |
| NO: 694 | dedicator of cytokinesis 2 (DOCK2), | ATGTATCCAAGTGTT |
| | RefSeqGene on chromosome 5 | CAATGGTAAGAGGAG |
| | | AGCCAAGGAGGCTTA |
| SEQ ID | GI 160358379 NG_007100 Homo sapiens | AAATGGGACAAGATG |
| NO: 695 | methylmalonyl-CoA mutase (MUT), | GCCATGACAGAGGAG |
| | RefSeqGene on chromosome 6 | CAAAAGTTATTGCTA |
| | | CAGGATTTGCTGATC |
| SEQ ID | GI 300796044 NG_023367 Homo sapiens | AGACATCTGTTTAAA |
| NO: 696 | platelet derived growth factor receptor | CCCCACAGTGTATGT |
| | beta (PDGFRB), RefSeqGene on | CTGTATGCATGTACC |
| | chromosome 5 | GGTGGGTACACAATT |
| SEQ ID | GI 11545840 NM_022120 Homo sapiens 3- | AGAAATACGAGAAAC |
| NO: 697 | oxoacid CoA-transferase 2 (OXCT2), | GAATTGAGCGCTTAA |
| | mRNA | CGATCCTGAAAGAGG |
| | | AAGATGGAGACGCTG |
| SEQ ID | GI 94557300 NM_006251 Homo sapiens | CTATCGATCTTGCCA |
| NO: 698 | protein kinase AMP-activated catalytic | AAGGAGTGATTCAGA |
| | subunit alpha 1 (PRKAA1), transcript | TGCTGAGGCTCAAGG |
| | variant 1, mRNA | AAAATCCTCAGAAGT |
| SEQ ID | GI 48928023 NM_003225 Homo sapiens | CCTGGTGCTTCTATC |
| NO: 699 | trefoil factor 1 (TFF1), mRNA | CTAATACCATCGACG |
| | | TCCCTCCAGAAGAGG |
| | | AGTGTGAATTTTAGA |
| SEQ ID | GI 237820672 NG_012057 Homo sapiens | AGAGACAAAACTAGA |
| NO: 700 | carboxylesterase 1 (CES1), | GCACAATTCTCCTGA |
| | RefSeqGene on chromosome 16 | TGTTGTCCCACAAAG |
| | | ATATCACCCCTGAGC |
| SEQ ID | GI 223972691 NG_009823 Homo sapiens | TCTTCTCCACGAGTT |
| NO: 701 | crystallin alpha A (CRYAA), | TTAACAAGCATGCGT |
| | RefSeqGene on chromosome 21 | ACTTTTGTAAGTGGT |
| | | GAGTGTAACGGAGGT |
| SEQ ID | GI 193290117 NG_008157 Homo sapiens | TCTCTACTCCATGTA |
| NO: 702 | hydroxysteroid 17-beta dehydrogenase 3 | CTCAGCTTCCAAGGT |
| | (HSD17B3), RefSeqGene on chromosome 9 | GAGTGATGTCATGAC |
| | | TTCCAAGTCTCTTCC |
| SEQ ID | GI 323422857 NM_001565 Homo sapiens | GTTCTGACCCTGCTT |
| NO: 703 | C-X-C motif chemokine ligand 10 | CAAATATTTCCCTCA |
| | (CXCL10), mRNA | CCTTTCCCATCTTCC |
| | | AAGGGTACTAAGGAA |
| SEQ ID | GI 1002635078 NM_006256 Homo sapiens | TACATCTGCCAACCT |
| NO: 704 | protein kinase N2 (PKN2), transcript | AGTTCACCTTGGTTT |
| | variant 1, mRNA | TGTGTCTGCTGTAGA |
| | | AGGGAACCATAACTT |
| SEQ ID | GI 66394595 DQ068066 Homo sapiens | TCTTAGGACTGTCTC |
| NO: 705 | baculoviral lAP repeat-containing 2 | ACATGTTACAGGACA |
| | (BIRC2) gene, complete cds | CCATGCTTGTTTCAC |
| | | ATCCACTAACTGCTG |
| SEQ ID | GI 74418831 DQ185447 Homo sapiens | GCACTTAGGTGGTCT |
| NO: 706 | interferon, alpha 1 (IFNA1) gene, | TTGAAACCTGTCAGG |
| | complete cds | TTTCACATCTCTGCT |
| | | TTGAGTGAAAAGCTC |
| SEQ ID | GI 315013553 NG_027995 Homo sapiens | CCACTGCCACCACTT |
| NO: 707 | cystatin A (CSTA), RefSeqGene on | TCTATGCAGCTACTA |
| | chromosome 3 | CTGTTCCAGAATCAA |
| | | AGAACTTTGCCAAAG |
| SEQ ID | GI 190358530 NG_008050 Homo sapiens | GACAGAGGGACTTGA |
| NO: 708 | endothelin 3 (EDN3), RefSeqGene on | GGCTGGAAGGCAGTT |
| | chromosome 20 | TGATTTGTAGCAAGA |
| | | AAACTTGGTACAAAG |
| SEQ ID | GI 224809340 NG_011507 Homo sapiens | AAGGAATCGATTGCC |
| NO: 709 | glutamate ionotropic receptor NMDAtype | CTAAAACATACCTGT |
| | subunit 1 (GRIN1), RefSeqGene on | TCCACTGTTGGCCAC |
| | chromosome [symbol] | TTTTGCAATAGACAA |
| SEQ ID | GI 211064491 NG_008915 Homo sapiens heat | TTCCTAACTCCATGA |
| NO: 710 | shock protein family D (Hsp60) member 1 | CAGCTCCTCAGTAGA |
| | (HSPD1), RefSeqGene on chromosome 2 | GCTTTCCTCCTCAGA |
| | | AGCAACTAGAACAAT |
| SEQ ID | GI 38327624 NM_004077 Homo sapiens | ATATACCTGTCAGCG |
| NO: 711 | citrate synthase (CS), mRNA | AGAGTTTGCTCTGAA |
| | | ACACCTGCCTAATGA |
| | | CCCCATGTTTAAGTT |
| SEQ ID | GI 195963400 NM_005359 Homo sapiens SMAD | CACCGTAAGACCTCA |
| NO: 712 | family member 4 (SMAD4), mRNA | TTCCATGTTTGTCCA |
| | | GTGCCTTTCAGTGCA |
| | | TTATCAAAGGGAATC |
| SEQ ID | GI 54781368 AY791292 Homo sapiens cyclin | TCTAGATGTCAGCCA |
| NO: 713 | GI (CCNG1) gene, complete cds | AAGGTCTGTGGTTTG |
| | | AGACTAATTGAGTCT |
| | | GCACACGATAATGGC |
| SEQ ID | GI 662840 L39370 Homo sapiens heat shock | CAATTAAAACTGTAA |
| NO: 714 | protein 27 (HSPB1) gene, complete cds | GGTGGGGAAGGAGGA |
| | | GGAAATAAGAGAAGC |
| | | ACCTGAGGCTTGAGT |
| SEQ ID | GI 345478654 NG_029871 Homo sapiens | GACAATCACTACTGG |
| NO: 715 | activating transcription factor 3 (ATF3), | TACTTGGCTAAATAT |
| | RefSeqGene on chromosome 1 | TGCAGCCACCTCTTC |
| | | AGGCTACACATGTCA |
| SEQ ID | GI 347446697 NG_030008 Homo sapiens colony | TCCTTTGGGGAAAGT |
| NO: 716 | stimulating factor 1 (CSF1), RefSeqGene | CTTAAAATTGCCACT |
| | on chromosome 1 | GTACAAATGAGGAAA |
| | | GAGAGGCCCAAAAGG |
| SEQ ID | GI 211064488 NG_008914 Homo sapiens heat | TGATTTCCACTGGAT |
| NO: 717 | shock protein family E (HsplO) member 1 | TAGCACCTTTGCTAA |
| | (HSPE1), RefSeqGene on chromosome 2 | TCTTCTCGAAGCCTT |
| | | CCTTGGCTATAGAGC |
| SEQ ID | GI 393533306 NG_032816 Homo sapiens 5- | TACTGAGAAACTCCA |
| NO: 718 | hydroxytryptamine receptor 1A (HTRIA), | ACCATCAGCCTTCAG |
| | RefSeqGene on chromosome 5 | TAATGATAGGGCTTC |
| | | AAGGTTCACAGCAAA |
| SEQ ID | GI 1041818081 NG_051000 Homo sapiens | GCTAAGTCGCCTTGA |
| NO: 719 | pentraxin 3 (PTX3), RefSeqGene on | GATGGACAATGGTGG |
| | chromosome 3 | AAACTTACTGAGCTT |
| | | TCCACCTCAAATTTG |
| SEQ ID | GI 1024336732 NM_006218 Homo sapiens | ACACTGCACTGTTAA |
| NO: 720 | phosphatidylinositol-4,5-bisphosphate 3- | TAACTCTCAGCAGGC |
| | kinase catalytic subunit alpha (PIK3CA), | AAAGACCGATTGCAT |
| | mRNA | AGGAATTGCACAATC |
| SEQ ID | GI 1073857156 NM_016930 Homo sapiens | CAGGAAGCTGAGATT |
| NO: 721 | syntaxin 18 (STX18), transcript variant | GACAGCATTCACCAG |
| | 1, mRNA | TTAGTTGTGGGGGCA |
| | | ACTGAAAATATCAAG |
| SEQ ID | GI 226532444 NG_011794 Homo sapiens brain | GCCCTTTGTGCATGT |
| NO: 722 | derived neurotrophic factor (BDNF), | TAGTTACAGCATGTA |
| | RefSeqGene on chromosome 11 | CCACATTGCACTGTA |
| | | TTGTTGGTTTTTGGG |
| SEQ ID | GI 260593637 NG_013005 Homo sapiens | AGAAAGGGGTCTTTG |
| NO: 723 | calcitonin receptor (CALCR), RefSeqGene | TGAACTGTACAGGCT |
| | on chromosome 7 | GTCTAGGATATTCAC |
| | | CAGATCACCTCATCT |
| SEQ ID | GI 291575141 NG_016810 Homo sapiens | TATCCTCTACATCCA |
| NO: 724 | nuclear receptor coactivator 3 (NCOA3), | ATATGCATGGGTCAC |
| | RefSeqGene on chromosome 20 | TGTTACAAGAGAAGC |
| | | ACCGGATTTTGCACA |
| SEQ ID | GI 332164677 NG_028910 Homo sapiens | GAAGGAAGGAAGGAG |
| NO: 725 | nuclear receptor subfamily 4 group A | GACACATCAGAGCTT |
| | member 3 (NR4A3), RefSeqGene on | TGGGACCTTACAGAT |
| | chromosome 9 | AATGAAGTTCAACCG |
| SEQ ID | GI 122937306 NM_001080454 Homo sapiens | CTGTGTTTTTGTGCA |
| NO: 726 | acyl-CoA synthetase medium chain family | TCGAATGGCACAGTT |
| | member 4 (ACSM4), mRNA | TGACACTGACACCTT |
| | | CCTAGACACACTTAC |
| SEQ ID | GI 68303552 NM_005165 Homo sapiens | GAGGCGTCATTTTCT |
| NO: 727 | aldolase, fructose-bisphosphate C | TCCATGAGACCCTCT |
| | (ALDOC), mRNA | ACCAGAAAGATGATA |
| | | ATGGTGTTCCCTTCG |
| SEQ ID | GI 538917457 NM_005896 Homo sapiens | CCAAGGATGCTGCAG |
| NO: 728 | isocitrate dehydrogenase (NADP(+)) 1, | AAGCTATAAAGAAGC |
| | cytosolic (IDH1), transcript variant 1, | ATAATGTTGGCGTCA |
| | mRNA | AATGTGCCACTATCA |
| SEQ ID | GI 21927971 AY113189 Homo sapiens OCT-1 | TACACTGGCAACTAT |
| NO: 729 | protein (POU2F1) mRNA, complete cds | TCAAGCTCTTGCTTC |
| | | TGGTGGCTCTCTTCC |
| | | AATAACATCACTTGA |
| SEQ ID | GI 593023777 KJ145927 Homo sapiens lamin | CTCACCTTCACTTCT |
| NO: 730 | A/C (LMNA) gene, complete cds | CAGGGCTTTGGTTTT |
| | | CCCATTCGAAAATGG |
| | | AGGCTGTTCTTAATC |
| SEQ ID | GI 209447054 NG_008716 Homo sapiens | AATTAGAGGGATTAG |
| NO: 731 | aminolevulinate dehydratase (ALAD), | ACCTGGCTTCGACAT |
| | RefSeqGene on chromosome 9 | CTGACTTGACTCCAA |
| | | CCTACCAAAGGATCT |
| SEQ ID | GI 1024336777 NG_046929 Homo sapiens | TTGTCATCTCTGGGG |
| NO: 732 | BTG anti-proliferation factor 2 | CTGTCATTTGCTATC |
| | (BTG2), RefSeqGene on chromosome 1 | CACCATACTTGTCTT |
| | | GTTAGACAAGAGCAG |
| SEQ ID | GI 297515495 NG_021405 Homo sapiens MHC | TTTCCATTGCAGGCG |
| NO: 733 | class 1 polypeptide-related sequence B | TGCCATTTTGTATTT |
| | (MICB), RefSeqGene on chromosome 6 | CTAGAAGCAGTATAC |
| | | AGGGGCTTCAGTTTC |
| SEQ ID | GI 238018099 NG_012213 Homo sapiens | CCTCTTTCTCTGATA |
| NO: 734 | superoxide dismutase 3 (SOD3), | ACACTGTCCTCTTTC |
| | RefSeqGene on chromosome 4 | TCTGATAACACCGTC |
| | | CTCCCTTCCAGATCC |
| SEQ ID | GI 53759124 NM_001295 Homo sapiens C-C | TTGTCAACAAAGTCA |
| NO: 735 | motif chemokine receptor 1 (CCR1), mRNA | CCCACTTCCCACTAT |
| | | TGCTTGCACAAACCA |
| | | ATTAAACCCAGTAGT |
| SEQ ID | GI 798958230 NG_042077 Homo sapiens | CGGTCACAATAGCAG |
| NO: 736 | enoyl-CoA hydratase, short chain 1 | TTTCGCTTTCCATTC |
| | (ECHS1), RefSeqGene on chromosome 10 | CATTTTATCCAGGGT |
| | | GGTTTTATCTCCACC |
| SEQ ID | GI 318064850 NG_028074 Homo sapiens ras | ATCACTGATGAGGAA |
| NO: 737 | related dexamethasone induced 1 | TCTACCAGAGTTAAA |
| | (RASD1), RefSeqGene on chromosome 17 | ATCTCAGCTTGATGA |
| | | GGGGAGCCTGTCAGA |
| SEQ ID | GI 310703611 NG_027845 Homo sapiens | GAGAAAAGTCACACA |
| NO: 738 | clusterin (CLU), RefSeqGene on | AGATCCACCAGGCCT |
| | chromosome 8 | CAGACCTCTGATTCC |
| | | ACAGTCTCATTTTAC |
| SEQ ID | GI 1024336774 NG_046918 Homo sapiens | TTTGTGGACAATTGC |
| NO: 739 | DExD/H-box helicase 58 (DDX58), | AATCCTGAATCCTGC |
| | RefSeqGene on chromosome 9 | ACATCTGCTTCACAG |
| | | TTTTCCCTTTACAGA |
| SEQ ID | GI 226693358 NG_011855 Homo sapiens | TCTTCAGCCTGACTG |
| NO: 740 | heat shock protein family A (Hsp70) | GAATTTACTGCCGGT |
| | member 1 like (HSPAlL), RefSeqGene | GAACCCCTAATAGAG |
| | on chromosome [symbol] | AATATTATCCCAGGA |
| SEQ ID | GI 170932506 NG_007486 Homo sapiens | TTTGGAGGGACACAA |
| NO: 741 | protein kinase AMP-activated non- | TTCAACCCATGACAT |
| | catalytic subunit gamma 2 (PRKAG2), | AGGACTATCAAGGAA |
| | RefSeqGene on chr [symbol] | TGGATATGTCCTGCC |
| SEQ ID | GI 90657251 DQ451402 Homo sapiens heat | TCCTGGGATAATATT |
| NO: 742 | shock 70 kDa protein 1A (HSPA1A) gene, | CTCTATTAGGGGTTC |
| | complete cds | ACCGGCAGTAAATTC |
| | | CAGTCAGGCTGAAGA |
| SEQ ID | GI 325053739 NG_028167 Homo sapiens | GCTCATAGTAGTCTA |
| NO: 743 | growth differentiation factor 3 (GDF3), | TTCCTTCTAGAGCAT |
| | RefSeqGene on chromosome 12 | GCACTCTTGTCTCAT |
| | | CCTCACACGTGCTGT |
| SEQ ID | GI 196259816 NG_008365 Homo sapiens | AGGACATGTAAGTGG |
| NO: 744 | steroid 5 alpha-reductase 2 (SRD5A2), | AGTTTTCCAGCAGGG |
| | RefSeqGene on chromosome 2 | AGAAAACGCAGAGCT |
| | | AAAGCTCATATCTTT |
| SEQ ID | GI 1150561157 NG_052945 Homo sapiens | ATTAATGGGGCTGCA |
| NO: 745 | succinate-CoA ligase GDP-forming beta | GGAGAAGTGAGAAAG |
| | subunit (SUCLG2), RefSeqGene on chromosome 3 | AGATCAGCTGTCATT |
| | | ATGGTCTTCCAAGTG |
| SEQ ID | GI 341865593 NG_029549 Homo sapiens | ACAGGTTTTGAAGCT |
| NO: 746 | TGF-beta activated kinase 1 (MAP3K7) | GAAAAGTCGATGTAG |
| | binding protein 1 (TAB1), RefSeqGene | AGTGGCAACTGACTT |
| | on chromosom | AAGGGACACAGAAGT |
| SEQ ID | GI 193083137 NM_005512 Homo sapiens | TTGGTATAGTGGGAA |
| NO: 747 | leucine rich repeat containing 32 | CATTTGCTTTGGAGA |
| | (LRRC32), transcript variant 1, mRNA | CAGATGAACTGGATT |
| | | CTGATCGTGACCCTG |
| SEQ ID | GI 157909838 NM_006252 Homo sapiens | CCTCCTACAAATGAT |
| NO: 748 | protein kinase AMP-activated catalytic | CATGTTGTTTGCATC |
| | subunit alpha 2 (PRKAA2), mRNA | TGCCTGATTGAGTTC |
| | | CCTTGGTCAAACCTC |
| SEQ ID | GI 88698111 DQ388429 Homo sapiens heat | AGTTTGCTTAAAGAG |
| NO: 749 | shock 70 kDa protein 1B (HSPA1B) gene, | GGCCACCTGCCAAAC |
| | complete cds | AGCTGTAGTATGTGA |
| | | TGTTAAAGAGAGCTA |
| SEQ ID | GI 117606493 EF064721 Homo sapiens | TTCTCCTTCCCTCCT |
| NO: 750 | interleukin 7 (IL7) gene, complete cds | CTTCCTTGTTCACGC |
| | | TATAGCAGAATGTGA |
| | | CATACTCCTTACCAG |
| SEQ ID | GI 34732733 AY388493 Homo sapiens | ACTCACCAGGACCTT |
| NO: 751 | microsomal glutathione S-transferase | CCACTTCCATTTGTT |
| | 3 (MGST3) gene, complete cds | AGATGTGAGGTCCTT |
| | | TTACTCAGCATGTTC |
| SEQ ID | GI 340523104 NG_029469 Homo sapiens | GACTCTACTTTGGCA |
| NO: 752 | heat shock protein family A (Hsp70) | TTGGAGTTAGTGTCA |
| | member 9 (HSPA9), RefSeqGene on | AAGGGAAGGAATCCA |
| | chromosome 5 | GAAACCACCAGTAGG |
| SEQ ID | GI 238776810 NG_012247 Homo sapiens | AAGTTTATTGCCAGG |
| NO: 753 | solute carrier family 25 member 13 | CAACTGAGTAAGGGG |
| | (SLC25A13), RefSeqGene on chromosome | AGAGGAGTCACACTC |
| | 7 | AAGTTTGACTTTCTC |
| SEQ ID | GI 375331875 NM_000895 Homo sapiens | GAACAAGGAAGAATG |
| NO: 754 | leukotriene A4 hydrolase (LTA4H), | AAGTTTACCCGGCCC |
| | transcript variant 1, mRNA | TTATTCAAGGATCTT |
| | | GCTGCCTTTGACAAA |
| SEQ ID | GI 221136810 NM_006931 Homo sapiens | CCTATGCCGAATGCC |
| NO: 755 | solute carrier family 2 member 3 | CTCAGGGGACTTGAA |
| | (SLC2A3), mRNA | TCTTTCCAATAAACC |
| | | AGGTTTAGACAGTAT |
| SEQ ID | GI 751368105 NM_145911 Homo sapiens | CTATATGTGTAGTGT |
| NO: 756 | zinc finger protein 23 (ZNF23), | GTGTGGGAAAGCATT |
| | transcript variant 1, MRNA | CAGGTTTAGCTTCCA |
| | | GCTCAGTCAGCATCA |
| SEQ ID | GI 1049010976 AH002711 Homo sapiens | ACTGTATAAAGAGGC |
| NO: 757 | platelet glycoprotein Illa (ITGB3) | CACGTCTACCTTCAC |
| | gene, complete cds | CAATATCACGTACCG |
| | | GGGCACTTAATGATA |
| SEQ ID | GI 379642970 NG_032137 Homo sapiens | CTCTTTAAAGGCTGA |
| NO: 758 | collagen type IV alpha 2 chain | GTCCTATTCCATTGT |
| | (COL4A2), RefSeqGene on chromosome 13 | GTGGGTGTCTACCCA |
| | | TTGTTGGACACTTGA |
| SEQ ID | GI 1024336768 NG_046906 Homo sapiens | CCACAAAACCAGGGA |
| NO: 759 | TANK binding kinase 1 (TBK1), | CTTTCATCTTAGCCT |
| | RefSeqGene on chromosome 12 | ACCGAGAACACCAAG |
| | | CAGAATTAGACAAGA |
| SEQ ID | GI 307775406 NM_015364 Homo sapiens | TGTTGTTGAAGCTAT |
| NO: 760 | lymphocyte antigen 96 (LY96), | TTCTGGGAGCCCAGA |
| | transcript variant 1, mRNA | AGAAATGCTCTTTTG |
| | | CTTGGAGTTTGTCAT |
| SEQ ID | GI 61744461 NM_003405 Homo sapiens | CGTTAAGCAGTACAT |
| NO: 761 | tyrosine 3-monooxygenase/tryptophan | CTTGTGCATGCAAAA |
| | 5-monooxygenase activation protein | ATGAATTCACCCCTC |
| | eta (YWHAH), [symbol] | CCACCTCTTTCTTCA |
| SEQ ID | GI 187473251 EU600171 Homo sapiens v- | TCTTACTAGGGAAAG |
| NO: 762 | raf murine sarcoma viral oncogene | CCATCAGTACTCTGT |
| | homolog B1 (BRAF) gene, complete cds | GGCTGATCAGCCTTT |
| | | TCTTGCATGAAACAA |
| SEQ ID | GI 237874281 NG_012193 Homo sapiens | AATCAGACCCAATCA |
| NO: 763 | cytochrome c oxidase subunit 6B1 | TGAGAGTATATCTGC |
| | (COX6B1), RefSeqGene on chromosome 19 | TGTGTCCCCTTGAAG |
| | | AGAAGACTCAGACCC |
| SEQ ID | GI 383209671 NG_032573 Homo sapiens | AAGTAACCTCTTACC |
| NO: 764 | DnaJ heat shock protein family | CAAAACTTCCAGGGA |
| | (Hsp40) member B6 (DNAJB6), | TGCTTTACATAATGC |
| | RefSeqGene on chromosome | CAGGCCTCTGAGAAA |
| SEQ ID | GI 983173900 NG_009770 Homo sapiens | AGGGATGGGTTATGA |
| NO: 765 | hexosaminidase subunit beta (HEXB), | AATCAAACTTGTCAG |
| | RefSeqGene on chromosome 5 | TTGGGATCCTTTATA |
| | | TCGGGACCCACACAG |
| SEQ ID | GI 237858685 NG_012086 Homo sapiens | GACTCCATCCTACTT |
| NO: 766 | interleukin 4 receptor (IL4R), | TTCTTCCCATGTATT |
| | RefSeqGene on chromosome 16 | CTTTCCTGCTTGTAC |
| | | CAGTTCACTGGGGTG |
| SEQ ID | GI 1008909437 NG_023257 Homo sapiens | ACATTTCCTGCTATT |
| NO: 767 | lactotransferrin (LTF), RefSeqGene | ACTCTCATGGAAAGG |
| | on chromosome 3 | CCATGATGCTGTCAA |
| | | CAATTGACCACAAGC |
| SEQ ID | GI 970414659 NG_013325 Homo sapiens | GGACATTAACTCTTC |
| NO: 768 | mitogen-activated protein kinase 10 | TAGTACTTCAGAATG |
| | (MAPK10), RefSeqGene on chromosome 4 | GGCTTGTTCAAGGGC |
| | | TGAGCAGGTTTCCTT |
| SEQ ID | GI 212549709 NG_008958 Homo sapiens | TAGAAGTAATTTTAA |
| NO: 769 | minichromosome maintenance complex | GGGCTGAAGCTGTGG |
| | component 6 (MCM6), RefSeqGene on | AATCAGCTCAAGCTG |
| | chromosom [symbol] | GTGACAAGTGTGACT |
| SEQ ID | GI 193211428 NG_008124 Homo sapiens | ATATCTATCTACCCG |
| NO: 770 | msh homeobox 2 (MSX2), RefSeqGene | ACTTCCTGGGCTGTG |
| | on chromosome 5 | TAACTCGGAAAGTGA |
| | | TCTCTCTAGACACCA |
| SEQ ID | GI 300388175 NG_011471 Homo sapiens | AAGTGACAGAAACCC |
| NO: 771 | purinergic receptor P2X 7 (P2RX7), | AACTTGCGTTAACCA |
| | RefSeqGene on chromosome 12 | CAAAGCCTCAAAGGG |
| | | GAATTTATTGGATCA |
| SEQ ID | GI 300244554 NG_023330 Homo sapiens | TAACTCATGCATTTG |
| NO: 772 | SMAD family member 7 (SMAD7), | AGCTCTTTTTTTGCT |
| | RefSeqGene on chromosome 18 | TTTGCTCTTTGCGCA |
| | | TGGGGGATGTAAAGG |
| SEQ ID | GI 78482607 NM_021129 Homo sapiens | TCTAGGCATATTGGC |
| NO: 773 | pyrophosphatase (inorganic) 1 (PPA1), | TATGATTGACGAAGG |
| | mRNA | GGAAACCGACTGGAA |
| | | AGTCATTGCCATTAA |
| SEQ ID | GI 662033938 NG_023295 Homo sapiens | GCCTCTGTCATGTCA |
| NO: 774 | acetyl-CoA carboxylase alpha (ACACA), | TCTGATTGTGCTTGC |
| | RefSeqGene on chromosome 17 | CCCATAGTTAGTGTA |
| | | CTGTGAAAAAGAGGA |
| SEQ ID | GI 226958454 NG_011902 Homo sapiens | TGGCCTGCTATGTGG |
| NO: 775 | CCHC-type zinc finger nucleic acid | AGAACGCTCTTTCCT |
| | binding protein (CNBP), RefSeqGene | TACTGTCTCACTTAA |
| | on chromosome 3 | TAGAACTCCTGTTCT |
| SEQ ID | GI 222144288 NG_009492 Homo sapiens | TTTGCCTCTGTAAGT |
| NO: 776 | insulin like growth factor 1 receptor | ATCTCCATATTCCTC |
| | (IGF1R), RefSeqGene on chromosome 15 | CATCCTTTCAGGTCC |
| | | CATTCAGTCCCTAGG |
| SEQ ID | GI 161086962 NG_007114 Homo sapiens | GGGGTCAGGTGCACT |
| NO: 777 | insulin (INS), RefSeqGene on | TTTTTAAAAAGAAGT |
| | chromosome 11 | TCTCTTGGTCACGTC |
| | | CTAAAAGTGACCAGC |
| SEQ ID | GI 237858728 NG_012105 Homo sapiens | GTGTCACCCCCTTTT |
| NO: 778 | ornithine decarboxylase 1 (ODC1), | ATGCAGTCAAATGTA |
| | RefSeqGene on chromosome 2 | ATGATAGCAAAGCCA |
| | | TCGTGAAGACCCTTG |
| SEQ ID | GI 289577082 NG_016598 Homo sapiens | TGAAAGCTATGGAAT |
| NO: 779 | phosphorylase kinase regulatory | TGCTCTTCAGACGTG |
| | subunit beta (PHKB), RefSeqGene on | TGCACAGGATTTTGG |
| | chromosome 16 | AAACAATTTCTGGGT |
| SEQ ID | GI 45243468 NM_002934 Homo sapiens | CTGTCCTAGTAACAA |
| NO: 780 | ribonuclease A family member 2 | AACTCGCAAAAATTG |
| | (RNASE2), mRNA | TCACCACAGTGGAAG |
| | | CCAGGTGCCTTTAAT |
| SEQ ID | GI 22122007 AY135686 Homo sapiens | TGACTAATTCCTTTA |
| NO: 781 | growth arrest and DNA-damage- | GCAGGGCAGATTAGA |
| | inducible, alpha (GADD45A) gene, | TAAAGCCAAATGAAT |
| | complete cds | TCCTGGCTCACCCCT |
| SEQ ID | GI 4588599 AF111163 Homo sapiens | GTTGAGGACCAGCAT |
| NO: 782 | pyrin (MEFV) gene, complete cds | TTAGACCTCTGAATC |
| | | CAGGGAAGACTTTTC |
| | | TTTGTGTAGCTCAGG |
| SEQ ID | GI 60391308 D50017 Homo sapiens | ATATTTATGTGGACC |
| NO: 783 | PDGFRA gene for alpha-platelet- | CGATGCAGCTGCCTT |
| | derived growth factor receptor, | ATGACTCAAGATGGG |
| | complete cds | AGTTTCCAAGAGATG |
| SEQ ID | GI 922304389 NG_042830 Homo sapiens | TGCTTTACTTCCTGA |
| NO: 784 | acyl-CoA dehydrogenase family member | TGTCAAAATGAGCTG |
| | 11 (ACAD11), RefSeqGene on | CATAGCTTCCCCTCC |
| | chromosome 3 | TTAGAATGGTCAGTC |
| SEQ ID | GI 261598995 NG_013210 Homo sapiens | ACTCCAGACCTACAA |
| NO: 785 | aspartate beta-hydroxylase (ASPH), | AATCAGAAACTCTGG |
| | RefSeqGene on chromosome 8 | GGTTAAGGTCGGCAT |
| | | CATGTCTTAACAAGC |
| SEQ ID | GI 219689119 NG_009261 Homo sapiens | ATATGTCTGCTCACA |
| NO: 786 | elastin (ELN), RefSeqGene on | GGTTCATCCTTAGGC |
| | chromosome 7 | AGGTGTATGCGTATG |
| | | TTTGGAGGAGGTATA |
| SEQ ID | GI 347658934 NG_030028 Homo sapiens | CCTGGGTATGTACAC |
| NO: 787 | mitochondrial antiviral signaling | CACAGAAAGCTATGT |
| | protein (MAVS), RefSeqGene on | CCACCGAGACATTGG |
| | chromosome 20 | CAAGAATGTTTCTAA |
| SEQ ID | GI 296434305 NG_021272 Homo sapiens | ATTGTTAGAAGAGGC |
| NO: 788 | signal transducer and activator of | TCTTCTGTGAGAAAG |
| | transcription 6 (STAT6), RefSeqGene | GAGCAGCAAATGCTA |
| | on chromosome 12 | ACAGCCTGTCTTCTT |
| SEQ ID | GI 225703119 NG_011705 Homo sapiens | CACAATTTGGCTCAT |
| NO: 789 | tyrosinase related protein 1 (TYRP1), | CTATTCCTGAATGGA |
| | RefSeqGene on chromosome 9 | ACAGGGGGACAAACC |
| | | CATTTGTCTCCAAAT |
| SEQ ID | GI 375331875 NM_000895 Homo sapiens | CACCATCCTTCCCTT |
| NO: 790 | leukotriene A4 hydrolase (LTA4H), | ATGGTGGCATGGAGA |
| | transcript variant 1, mRNA | ATCCTTGCCTTACTT |
| | | TTGTAACTCCTACTC |
| SEQ ID | GI 156616272 NM_002664 Homo sapiens | AGTGGAGAGCAACTC |
| NO: 791 | pleckstrin (PLEK), mRNA | AAATGGCAGGAAGAG |
| | | TGAGGAAGAGAACCT |
| | | TTTTGAGATCATCAC |
| SEQ ID | GI 38016923 NM_006253 Homo sapiens protein | GAAGTTTACTTATCT |
| NO: 792 | kinase AMP-activated non-catalytic subunit | GGGTCCTTCAACAAC |
| | beta 1 (PRKAB1), mRNA | TGGAGTAAACTTCCC |
| | | CTCACCAGAAGCCAC |
| SEQ ID | GI 117606677 EF078888 Homo sapiens Kruppel- | CCCAATGACTTTTTC |
| NO: 793 | like factor 2 (lung) (KLF2) gene, complete | ACATTAGCTCACATT |
| | cds | TCACCCCCTGGGTGA |
| | | GATTCAGAATTTCCC |
| SEQ ID | GI 584458490 NG_012645 Homo sapiens FK506 | TAAGCTGCTTAGGCT |
| NO: 794 | binding protein 5 (FKBP5), RefSeqGene on | TGAAACGTGCTGTGT |
| | chromosome 6 | TCTTTTACCAGCCTT |
| | | GATTTTACTCTTGCC |
| SEQ ID | GI 226693358 NG_011855 Homo sapiens heat | GCTTTAAGGATTGTA |
| NO: 795 | shock protein family A (Hsp70) member 1 | GTGGGCGTTTTCCAA |
| | like (HSPAlL), RefSeqGene on chromosome | GGAGGAGGGAATTAC |
| | [symbol] | TTCTAGGATGCTAGC |
| SEQ ID | GI 340523104 NG_029469 Homo sapiens heat | GAACATTGCAACGTT |
| NO: 796 | shock protein family A (Hsp70) member 9 | GGTTAGGGTTGAGGT |
| | (HSPA9), RefSeqGene on chromosome 5 | TGAAATACCTGGCAC |
| | | TGCCAGAAGATAATT |
| SEQ ID | GI 211064488 NG_008914 Homo sapiens heat | CATTGTTCTAGTTGC |
| NO: 797 | shock protein family E (HsplO) member 1 | TTCTGAGGAGGAAAG |
| | (HSPEl), RefSeqGene on chromosome 2 | CTCTACTGAGGAGCT |
| | | GTCATGGAGTTAGGA |
| SEQ ID | GI 224809245 NG_011450 Homo sapiens | CCCTTACAAACATAC |
| NO: 798 | interferon regulatory factor 1 (IRF1), | ACAGCACTCTGCAGG |
| | RefSeqGene on chromosome 5 | GCTCCAATCGAACAA |
| | | ATAGAAGACTGAGAA |
| SEQ ID | GI 345842418 NG_029946 Homo sapiens SMAD | TGCATTTGGACTTCC |
| NO: 799 | family member 2 (SMAD2), RefSeqGene on | AACCTCCACAGGACT |
| | chromosome 18 | CCATCACACATTACT |
| | | GTCACATCAGATTAC |
| SEQ ID | GI 576795860 NM_001549 Homo sapiens | GCAGGGAAGCTTTGC |
| NO: 800 | interferon induced protein with | ATGTTGCTCTAAGGT |
| | tetratricopeptide repeats 3 (IFIT3), | ACATTTTTAAAGAGT |
| | transcript variant 1, mR [symbol] | TGTTTTTTGGCCGGG |
| SEQ ID | GI 117606551 EF064750 Homo sapiens CD80 | GCCATATCCACACTT |
| NO: 801 | molecule (CD80) gene, complete cds | AGTTCTTTGCCAGAC |
| | | TTGTTCTGTGTTTCC |
| | | AGTAGCTAGAGGGTG |
| SEQ ID | GI 26000792 AY186092 Homo sapiens | CCCTCTGCCTGGAAT |
| NO: 802 | interleukin-1 receptor-associated | TTTCTTTTGGGCTTA |
| | kinase 4 (IRAK4) gene, complete cds | TCCTCATTCCCTTTC |
| | | ATTCATCCTTCCAAC |
| SEQ ID | GI 1047225988 NG_051241 Homo sapiens | CCAAAGGGAAAATGA |
| NO: 803 | arrestin domain containing 3 (ARRDC3), | AGGAAGTAAAACAGC |
| | RefSeqGene on chromosome 5 | TTGTGGCTAACTTGC |
| | | GTGGGGAATCCTTAT |
| SEQ ID | GI 209977070 NG_008850 Homo sapiens | ACAGAGAACTGGTCT |
| NO: 804 | interleukin l alpha (ILIA), RefSeqGene | CTCATTTGTCTCATC |
| | on chromosome 2 | CTGTTCTTTCTAGCA |
| | | GCCACGACTTTCCTA |
| SEQ ID | GI 209447066 NG_008725 Homo sapiens | GAGGGTAGAAGGCAA |
| NO: 805 | paraoxonase 2 (PON2), RefSeqGene on | ACCCTAACATTTTTT |
| | chromosome 7 | CCTGCTGTCACCCTC |
| | | CATCTTAGATGACTC |
| SEQ ID | GI 543173138 NM_018202 Homo sapiens | ACTTGTCCTCCTAGC |
| NO: 806 | transmembrane protein 57 (TMEM57), | AGATTTTGTCCTGGA |
| | transcript variant 1, mRNA | GTTCAGATTTGAATA |
| | | CCTGTGGCCATTCTG |
| SEQ ID | GI 270048011 GU143397 Homo sapiens | ATATCAAAAGAAGCA |
| NO: 807 | collagen, type III, alpha 1 (COL3A1) | TGGCCAACAGTGCCA |
| | gene, complete cds | GAAATGGCATCCAGT |
| | | GTCTTGTGTTTATGA |
| SEQ ID | GI 15128210 AF402002 Homo sapiens | GACACTCAATTTGGG |
| NO: 808 | interleukin 20 (IL20) gene, complete | AAGCTGTGTGATCGC |
| | cds | CACAAACCTTCAGGA |
| | | AATACGAAATGGATT |
| SEQ ID | GI 1048072306 NG_051267 Homo sapiens | GAGACCTAATAGAGA |
| NO: 809 | arsA arsenite transporter, ATP-binding, | TGCATGTTTATTGTG |
| | homolog 1 (bacterial) (ASNAl), RefSeqGene | GCAACTTTGGAAGGA |
| | on chr [symbol] | GGGTGTCTTGGGTGA |
| SEQ ID | GI 654825103 NG_029876 Homo sapiens C-C | TACTCTCCATCCCAG |
| NO: 810 | motif chemokine ligand 23 (CCL23), | ACGTGGTCATGGTAC |
| | RefSeqGene on chromosome 17 | TCATTTAAAAGCAGC |
| | | ACCAGAGTATAGACC |
| SEQ ID | GI 190358505 NG_008040 Homo sapiens | CTGGGACTTAGGCTT |
| NO: 811 | colony stimulating factor 2 receptor | CTGTTAATATGGCCC |
| | beta common subunit (CSF2RB), RefSeqGene | TTGGTTTCTTAGCTT |
| | on chrom [symbol] | TTGGGACTACCAAGT |
| SEQ ID | GI 344030205 NG_029797 Homo sapiens | GAGATGGGTGTACAC |
| NO: 812 | glutaredoxin (GLRX), RefSeqGene on | AATTTACAGGTAGAG |
| | chromosome 5 | TGAATGCAAAGGTCC |
| | | TGGGGTAGACGTTTG |
| SEQ ID | GI 1154491908 NG_052978 Homo sapiens | AAGCTTTTGCATTCA |
| NO: 813 | pyruvate kinase, muscle (PKM), | GGGGAATGACTTTGG |
| | | GTTTGGGTGAGGATT |
| | RefSeqGene on chromosome 15 | GTAAAGAGGCAATAT |
| SEQ ID | GI 31343534 NM_178818 Homo sapiens CKLF | GCTTTTTCCAGAATC |
| NO: 814 | like MARVEL transmembrane domain | GATTTTGCAAGCTTC |
| | containing 4 (CMTM4), transcript | AAGATTCTGTTCCCC |
| | variant 1, mRNA | TCTTCGCAGAAGTGA |
| SEQ ID | GI 197304767 NM_014674 Homo sapiens ER | TCTGAAATACTGGGT |
| NO: 815 | degradation enhancing alpha-mannosidase | AGAGAATGGCTGAGG |
| | like protein 1 (EDEM1), mRNA [symbol] | AGGAGATTACCAGAG |
| | | AAGTTGCTTTGCTCA |
| SEQ ID | GI 33867482 AY368173 Homo sapiens | CAGATTCCCTTATGG |
| NO: 816 | microsomal glutathione S-transferase 1 | CATATCATCTCCAGG |
| | (MGST1) gene, complete cds | TTAACTCTGGATCTG |
| | | GGTTCCTGCCTCTAT |
| SEQ ID | GI 292658840 NG_016861 Homo sapiens | CAGGACACCGATGCA |
| NO: 817 | C-X-C motif chemokine ligand 12 (CXCL12), | AACCAAAATAAACTG |
| | RefSeqGene on chromosome 10 | TTGCCTCTGAACACA |
| | | CAAAACAAAACCCTG |
| SEQ ID | GI 1068937310 NG_008847 Homo sapiens | AAACCTAAAGTTGTG |
| NO: 818 | glucokinase (GCK), RefSeqGene on | AATGGCGGAAGCACA |
| | chromosome 7 | AATTTCCCAAGTGGA |
| | | TCATGGAGAGTGATG |
| SEQ ID | GI 257467500 NG_012841 Homo sapiens | TGAAGGCTTGACTGG |
| NO: 819 | interleukin 13 receptor subunit alpha 1 | GGACAGAGCATCCAT |
| | (IL13RA1), RefSeqGene on chromosome X | TTCCAAGTTCACTCA |
| | | TATAGTTGTTGGAAA |
| SEQ ID | GI 1212890194 NG_032955 Homo sapiens | AGGATCAAAAGCCAA |
| NO: 820 | mitogen-activated protein kinase 8 | TCTGGAAAGTTGAGG |
| | interacting protein 2 (MAPK8IP2), | TCAAACTCTCTGTGA |
| | RefSeqGene on chro [symbol] | AATCTGGGAAGGGAT |
| SEQ ID | GI 34732733 AY388493 Homo sapiens | CTCGAACCTCTGTTA |
| NO: 821 | microsomal glutathione S-transferase | AAGCTGAAAGAAGAA |
| | 3 (MGST3) gene, complete cds | GTCAGTCTTCTGCTG |
| | | GGCACACTGTTAGTG |
| SEQ ID | GI 269914110 NG_015981 Homo sapiens | ATGGCAATGCAGCCA |
| NO: 822 | anti-Mullerian hormone receptor | TTGTGTGTCAACAGT |
| | type 2 (AMHR2), RefSeqGene on | TGTAGCAATACCTAT |
| | chromosome 12 | AGCATTTGGGACATT |
| SEQ ID | GI 340805888 NG_029485 Homo sapiens | TCTCCTGAGCTTTAG |
| NO: 823 | ATPase plasma membrane Ca2 | GTTTGAAAGCCCAAA |
| | transporting 1 (ATP2B1), RefSeqGene | ACACACAAAACCCAC |
| | on chromosome 12 | AGTGTTTAACTGGTT |
| SEQ ID | GI 193794846 NG_008174 Homo sapiens | ACCATTGAGGGTTTT |
| NO: 824 | fructose-bisphosphatase 1 (FBP1), | AATGTAGCCGGTCTT |
| | RefSeqGene on chromosome 9 | CTTCCCAGCTTCATA |
| | | ATTGACTCCACTTGT |
| SEQ ID | GI 211064491 NG_008915 Homo sapiens | CGCTCTATAGCCAAG |
| NO: 825 | heat shock protein family D (Hsp60) | GAAGGCTTCGAGAAG |
| | member 1 (HSPD1), RefSeqGene on | ATTAGCAAAGGTGCT |
| | chromosome 2 | AATCCAGTGGAAATC |
| SEQ ID | GI 514388123 NG_029053 Homo sapiens | AAACATCTTCAGTGC |
| NO: 826 | mitogen-activated protein kinase 8 | AATCCATTGTGTGTG |
| | (MAPK8), RefSeqGene on chromosome 10 | GTCTGGGATGACAAG |
| | | GGAAGAGATACTCTG |
| SEQ ID | GI 584458486 NG_012245 Homo sapiens | CGGCAGTGTCTGGAG |
| NO: 827 | N-acetyltransferase 1 (NAT1), | ACATTTTTGGTTGTT |
| | RefSeqGene on chromosome 8 | ACCACTAGGTAGTGC |
| | | TATTGGCATCTAGTG |
| SEQ ID | GI 224809324 NG_011504 Homo sapiens | CGAGTGTTCATAGGA |
| NO: 828 | NAD(P)H quinone dehydrogenase 1 | GAGTTTGCTTACACT |
| | (NQO1), RefSeqGene on chromosome 16 | TACGCTGCCATGTAT |
| | | GACAAAGGACCCTTC |
| SEQ ID | GI 257743484 NG_012879 Homo sapiens | GTCTCTCCTGGCTCT |
| NO: 829 | serpin family A member 3 (SERPINA3), | GAGTTCTATCTTCCA |
| | RefSeqGene on chromosome 14 | TTAAGGTTCTTCCTG |
| | | TTCCTCCCCTCATTC |
| SEQ ID | GI 4826562 Y17151 Homo sapiens Mrna | CCTGTAAAGTGCCTT |
| NO: 830 | for multidrug resistance protein 3 | ACAGGGTAACTGTGC |
| | (ABCC3) | TGAATGCTTTAGATG |
| | | AGGAAATGATCCCCA |
| SEQ ID | GI 211971007 NM_001136538 Homo sapiens | GGAACTTCTATATGG |
| NO: 831 | acyl-CoA dehydrogenase family member | CTTTTTCCTTTTTCC |
| | 10 (ACAD10), transcript variant 1, mRNA | GTGTGGCTGCAATCC |
| | | TACAGGGAGTCTACA |
| SEQ ID | GI 66933013 NM_020139 Homo sapiens | ATACAAGCCAGAGGA |
| NO: 832 | 3-hydroxybutyrate dehydrogenase 2 | AATCCTGAAGAGGCA |
| | (BDH2), mRNA | CGGAATGATTTCCTG |
| | | AAGAGACAAAAGACG |
| SEQ ID | GI 340745281 NG_029473 Homo sapiens | CAACATTGCACAATT |
| NO: 833 | heat shock protein family A (Hsp70) | CAAAATTCTAGGCAC |
| | member 8 (HSPA8), RefSeqGene on | CATTGTTTTGTGCAC |
| | chromosome 11 | CAGCTCAGTCACGTT |
| SEQ ID | GI 196259816 NG_008365 Homo sapiens | TTCTGCAATTTCTCC |
| NO: 834 | steroid 5 alpha-reductase 2 (SRD5A2), | CAATCTCACAGAGCT |
| | RefSeqGene on chromosome 2 | CTGCTATCCTGTAAC |
| | | AATCTGTTTACGCCC |
| SEQ ID | GI 41349436 NM_004329 Homo sapiens | ATTTATGTAACCAGT |
| NO: 835 | bone morphogenetic protein receptor | ATTTGCAACCCACAC |
| | type 1A (BMPR1A), mRNA | TGCCCCCTGTTGTCA |
| | | TAGGTCCGTTTTTTG |
| SEQ ID | GI 52694751 NM_001005340 Homo sapiens | ACTGCCAGATTAACA |
| NO: 836 | glycoprotein nmb (GPNMB), transcript | GATATGGCCACTTTC |
| | variant 1, mRNA | AAGCCACCATCACAA |
| | | TTGTAGAGGGAATCT |
| SEQ ID | GI 56157779 AY835629 Homo sapiens | GTGACTGCTGACAAA |
| NO: 837 | superoxide dismutase 1, soluble | GATGGTGTGGCCGAT |
| | (amyotrophic lateral sclerosis 1 | GTGTCTATTGAAGAT |
| | (adult)) (SOD1) gene, complete [symbol] | TCTGTGATCTCACTC |
| SEQ ID | GI 190194395 NG_008003 Homo sapiens | CAGAATACTGACGTC |
| NO: 838 | acyl-CoA dehydrogenase, short/ | TATAGGAGTGGGACC |
| | branched chain (ACADSB), RefSeqGene | CCTCCCTGGTGTCAC |
| | on chromosome 10 | TGCTGTAAAATTTTA |
| SEQ ID | GI 300193077 NG_023299 Homo sapiens | TCTGGTTTGAGACTG |
| NO: 839 | cAMP responsive element binding | GAACCACCAGGATTT |
| | protein 1 (CREB1), RefSeqGene on | TGATGCTGGTAAGGA |
| | chromosome 2 | TGTCTAAAATGGAGG |
| SEQ ID | GI 257153342 NG_012809 Homo sapiens | TTTTACGACCCTACA |
| NO: 840 | cysteinyl leukotriene receptor 1 | GGGTATCAAGATACT |
| | (CYSLTR1), RefSeqGene on chromosome X | GTGCAGCTCGCCAAC |
| | | AAGGATTAATTGCAA |
| SEQ ID | GI 1172222469 NG_053051 Homo sapiens | TTTTGCCACATTTTT |
| NO: 841 | heat shock protein family H (Hsp110) | TTTCCCCTGGTGAGC |
| | member 1 (HSPH1), RefSeqGene on | TGAGAAACTGATGGA |
| | chromosome 13 | CGAAGTGTTCTGATT |
| SEQ ID | GI 215490061 NG_009062 Homo sapiens | CCCCAGTACACAGAT |
| NO: 842 | nuclear receptor subfamily 3 group | TGTCTTCAGTATCTT |
| | C member 1 (NR3C1), RefSeqGene on chromosome 5 | CCCACTGACCCTCCA |
| | | GTACATATTCTGCAT |
| SEQ ID | GI 255652922 NG_012646 Homo sapiens | TCACTTTGGAGACAC |
| NO: 843 | transforming growth factor beta | CCCCTGTGTCTTCTA |
| | induced (TGFBI), RefSeqGene on | GATGTCAGACTTTCC |
| | chromosome 5 | AAATCTGTCTGCTAT |
| SEQ ID | GI 313569850 NM_144601 Homo sapiens | TCTGGGGGTGAGATG |
| NO: 844 | CKLF like MARVEL transmembrane domain | ACCATTCTGGGTCTA |
| | containing 3 (CMTM3), transcript | AGACTGTTTCAAAGA |
| | variant 1, mRN [symbol] | AGAGCTCATAGACTG |
| SEQ ID | GI 312261284 NM_016441 Homo sapiens | TTCCAAGCCAATCAA |
| NO: 845 | cysteine rich transmembrane BMP | TCAGCCAGTTCCTAG |
| | regulator 1 (CRIM1), mRNA | CAGAGTCAGCACATG |
| | | AACAAGATCTAAGTC |
| SEQ ID | GI 373432598 NM_001511 Homo sapiens | GGCCAATGAGATCAT |
| NO: 846 | C-X-C motif chemokine ligand 1 | TGTGAAGGCAGGGGA |
| | (CXCL1), transcript variant 1, | ATGTATGTGCACATC |
| | mRNA | TGTTTTGTAACTGTT |
| SEQ ID | GI 1005261235 NM_000399 Homo sapiens | ACTGAGCGAGGAGCA |
| NO: 847 | early growth response 2 (EGR2), | ATTGATTAATAGCTC |
| | transcript variant 1, mRNA | GGCGAGGGGACTCAC |
| | | TGACTGTTATAATAA |
| SEQ ID | GI 324072795 NM_005402 Homo sapiens | ATCCTTTGGTGAAAA |
| NO: 848 | RAS like proto-oncogene A (RALA), | CTGAGACACAAAATG |
| | mRNA | GCTGCAAATAAGCCC |
| SEQ ID | GI 570359552 NM_001288718 Homo sapiens | AAGGATGAGGTCTTC |
| NO: 849 | signal transducer and activator of | TCCAAGTACTACACT |
| | transcription 5A (STAT5A), transcript | CCTGTGCTGGCTAAA |
| | variant 1, mRNA | GCTGTTGATGGATAT |
| SEQ ID | GI 1024846757 NG_047018 Homo sapiens | TCTCTGAATTGCAGG |
| NO: 850 | histone deacetylase 1 (HDAC1), | GTCTTATTTCCCTCC |
| | RefSeqGene on chromosome 1 | AAGATGAGGCAAATT |
| | | CATGCCTTTCCTGTA |
| SEQ ID | GI 211904121 NG_008930 Homo sapiens | ACTGTTGAGTTCAGG |
| NO: 851 | cytochrome p450 oxidoreductase (POR), | ACACACCACGTGTAG |
| | RefSeqGene on chromosome 7 | CTGTGATCCAGGATA |
| | | AGAAGCTACTTCTAC |
| SEQ ID | GI 340805876 NG_029513 Homo sapiens | GCCAGCCCTACAAAA |
| NO: 852 | ribosomal protein 56 kinase B1 | ATAGGAGGTATAAGA |
| | (RPS6KB1), | ATGTTGCAGGCAGAA |
| | RefSeqGene on chromosome 17 | GAACAGCAAATGCAA |
| SEQ ID | GI 32815079 AY338491 Homo sapiens | TAACTATGTTGGACT |
| NO: 853 | cyclin B1 (CCNB1) gene, complete cds | ATGACATGGTGCACT |
| | | TTCCTCCTTCTCAAA |
| | | TTGCAGCAGGAGCTT |
| SEQ ID | GI 300193077 NG_023299 Homo sapiens | TTCCTGGAGGTTCTA |
| NO: 854 | cAMP responsive element binding | GGTGAAAATGTGTTT |
| | protein 1 (CREB1), RefSeqGene on | CTTTGCCATTTCTAG |
| | chromosome 2 | CTTCTAGAGGCCACC |
| SEQ ID | GI 262331544 NG_013350 Homo sapiens | CCTTGACAAGATCAA |
| NO: 855 | nuclear receptor subfamily 3 group C | GGCAACCTCAGAATA |
| | member 2 (NR3C2), RefSeqGene on | GAGGTTGCTGATGAG |
| | chromosome 4 | AACCTGGAATGAGTG |
| SEQ ID | GI 226693341 NG_011851 Homo sapiens | CTAGAGCGAAAAGCC |
| NO: 856 | synuclein alpha (SNCA), RefSeqGene on | TTAGGACCGCTTGTT |
| | chromosome 4 | TTAGACGGCTGGGGA |
| | | ATATTTATTCCTTGT |
| SEQ ID | GI 225703119 NG_011705 Homo sapiens | AGAATGAGTGCTCCT |
| NO: 857 | tyrosinase related protein 1 (TYRP1), | AAACTCCTCTCTCTG |
| | RefSeqGene on chromosome 9 | GGCTGTATCTTCTTC |
| | | CCCTTGCTACTTTTT |
| SEQ ID | GI 59853224 NM_001206 Homo sapiens | TCATGGAATGGTTCT |
| NO: 858 | Kruppel like factor 9 (KLF9), mRNA | GCTTATGATACTCTG |
| | | CACGCTGCCTTTTAG |
| | | TGAGTGAGGAGTTTG |
| SEQ ID | GI 669033292 NM_006798 Homo sapiens | TCTTATTGCCTCAGC |
| NO: 859 | UDP glucuronosyltransferase family 2 | CCTTGCCCAGATTCC |
| | member A1 complex locus (UGT2A1), | ACAGAAGGTTTTATG |
| | transcript variai [symbol] | GAGATACAAAGGAAA |
| SEQ ID | GI 66394595 DQ068066 Homo sapiens | AGCGTCAACTTCAAA |
| NO: 860 | baculoviral lAP repeat-containing 2 | AGTCGTTCGTTGGTT |
| | (BIRC2) gene, complete cds | GTCATACCACAAACA |
| | | TTTACTGCACGACAC |
| SEQ ID | GI 167887711 EF445033 Homo sapiens | AGTTCGTGATTCCCT |
| NO: 861 | signal transducing adapter molecule 1 | CCTCTTTTCTGAAAT |
| | (STAM) gene, complete cds, | GCTTTCTTCACTTGC |
| | alternatively spliced | TGGTGACCATTCTCT |
| SEQ ID | GI 190194387 NG_008002 Homo sapiens | TTGGGAAAGGGCCTT |
| NO: 862 | acyl-CoA dehydrogenase, long chain | TCCCAAGTAGCAGAA |
| | (ACADL), RefSeqGene on chromosome 2 | ACAAGATTTTCAAAG |
| | | ACTCATAGGTCAGGA |
| SEQ ID | GI 537544658 NG_033791 Homo sapiens | TGCTTTGAGAGTATG |
| NO: 863 | gremlin 1, DAN family BMP antagonist | TGTCCTCTGGGACAA |
| | (GREM1), RefSeqGene on chromosome 15 | GTGGAATGAAAGAGA |
| | | GGATAGAGGCAAAGA |
| SEQ ID | GI 334191656 NG_029012 Homo sapiens | TTCATGCTTCATTAA |
| NO: 864 | integrin subunit beta 1 (ITGB1), | GGCAGGCATTGCAAA |
| | RefSeqGene on chromosome 10 | CTAGTTCAGAGGAGA |
| | | GAATCCACGATGACA |
| SEQ ID | GI 188219645 NG_007730 Homo sapiens | AATTGGTAGGTTCGA |
| NO: 865 | protein tyrosine phosphatase, receptor | GGCACAGAAAGGTCA |
| | type C (PTPRC), RefSeqGene on | GGCATCTGTAGTATG |
| | chromosome 1 | GTGAGTAAGAACTGA |
| SEQ ID | GI 118722343 NM_030949 Homo sapiens | TAAGTGTCAAGGTGA |
| NO: 866 | protein phosphatase 1 regulatory | GAAAGCCTCACATTC |
| | inhibitor subunit 14C (PPP1R14C), mRNA | TCTCAAGACAGTTGC |
| | | TCTAGGAGCTGAGTT |
| SEQ ID | GI 423091980 JX512648 Homo sapiens | AAACAGGTTCTTGGA |
| NO: 867 | SH2 domain containing 1A (SH2D1A) | GTGCTGAGACAGCAC |
| | mRNA, complete cds | CTGGGGTACATAAAA |
| | | GATAATTCCGGAAAA |
| SEQ ID | GI 1028916196 KU928257 Homo sapiens | TTGTCCAGCACCACC |
| NO: 868 | complement decay-accelerating factor | ACAAATTGACAATGG |
| | (CD55) gene, CD55-203A allele, | AATAATTCAAGGGGA |
| | complete cds | ACGTGACCATTATGG |
| SEQ ID | GI 209977025 NG_008836 Homo sapiens | GCTTTTTCTACACTT |
| NO: 869 | angiotensinogen (AGT), RefSeqGene | ACACTTGCCTGGTAG |
| | on chromosome 1 | TTTTGGAGGGGGAGA |
| | | AAACATTGCAATCCG |
| SEQ ID | GI 340805888 NG_029485 Homo sapiens | GTTCCTAGTCACTCC |
| NO: 870 | ATPase plasma membrane Ca2 | ATGAGCAAGTCACTT |
| | transporting 1 (ATP2B1), RefSeqGene on | TATGTGCCTTGAAGC |
| | chromosome 12 | CTATTTCACCTTGGT |
| SEQ ID | GI 291575141 NG_016810 Homo sapiens | AGGCATGAAAACAGG |
| NO: 871 | nuclear receptor coactivator 3 (NCOA3), | AATGTTTGTTCTCAT |
| | RefSeqGene on chromosome 20 | TTAGGGTCATGGGTT |
| | | TTCAGGCTTGAGTGG |
| SEQ ID | GI 520260356 NM_021913 Homo sapiens | CCCTATACTGTAGTA |
| NO: 872 | AXL receptor tyrosine kinase (AXL), | TTCTTTGGGGTGCCC |
| | transcript variant 1, mRNA | CTCTCCTTCTTAGCT |
| | | ATCATTGCTTCCTCC |
| SEQ ID | GI 666335577 NM_006494 Homo sapiens | CCACAAGGTCACAGC |
| NO: 873 | ETS2 repressor factor (ERF), | AGGGGAGGGAGGGAC |
| | transcript variant 1, mRNA | AATTTTATAATGAAC |
| | | CAAAAATTCCATGTG |
| SEQ ID | GI 1020158924 NM_002895 Homo sapiens | GGACGGAGATATTTA |
| NO: 874 | RB transcriptional corepressor like 1 | CGAGAAAAAGAAGCA |
| | (RBL1), transcript variant 1, mRNA | GTCATTACTCCTGTT |
| | | GCATCAGCCACCCAA |
| SEQ ID | GI 2246432 U45984 Homo sapiens | GTAACCTGACACTCT |
| NO: 875 | CCR6 chemokine receptor (CMKBR6) gene, | CAGGAGACATTAAGA |
| | complete cds | TAGAAGGGGCTGTTC |
| | | TTCAGTGGTTCCCAT |
| SEQ ID | GI 224809428 NG_011537 Homo sapiens | GAGTGCAGAGACTGA |
| NO: 876 | myosin heavy chain 3 (MYH3), | GAAAGAGATGGCCAC |
| | RefSeqGene on chromosome 17 | CATGAAGGAAGAATT |
| | | CCAGAAAACCAAAGA |
| SEQ ID | GI 325652058 NM_005524 Homo sapiens | ACCTCTCTCCTTGGT |
| NO: 877 | hes family bHLH transcription factor 1 | CCTGGAACAGCGCTA |
| | (HES1), mRNA | CTGATCACCAAGTAG |
| | | CCACAAAATATAATA |
| SEQ ID | GI 599044742 NM_012234 Homo sapiens | GAGTTATCTAAGCAA |
| NO: 878 | RING1 and YYl binding protein (RYBP), | TCAGATGTTACAGGG |
| | mRNA | CCAGCCCTTTAGCTG |
| | | CTGTGGTGTATTCTG |
| SEQ ID | GI 221139857 NG_009364 Homo sapiens | CGGTGATGGATAAAA |
| NO: 879 | cyclin dependent kinase inhibitor 1A | TCACATCTGTGAAGC |
| | (CDKN1A), RefSeqGene on chromosome 6 | ATGGTGGGACACTCC |
| | | ATAATACCCCTCAAG |
| SEQ ID | GI 267844874 NG_015840 Homo sapiens | TGGCATGTCAGACAG |
| NO: 880 | interferon gamma (IFNG), RefSeqGene | AACTTGAATGTGTCA |
| | on chromosome 12 | GGTGACCCTGATGAA |
| | | AACATAGCATCTCAG |
| SEQ ID | GI 325651825 NG_028190 Homo sapiens | AGTCCTGACCTTTGT |
| NO: 881 | notch 4 (NOTCH4), RefSeqGene on | TGCGTTCCAGATAGA |
| | chromosome 6 | TACTTGCATCAGCCT |
| | | CCTAACTACAGTCTC |
| SEQ ID | GI 654825055 NG_028206 Homo sapiens | ATGAGTGTGGGATTT |
| NO: 882 | prostaglandin-endoperoxide synthase 2 | GACCAGTATAAGTGC |
| | (PTGS2), RefSeqGene on chromosome 1 | GATTGTACCCGGACA |
| | | GGATTCTATGGAGAA |
| SEQ ID | GI 1050193335 NG_027721 Homo sapiens | CTGCAGGAAAAGTGC |
| NO: 883 | transforming growth factor beta 2 | AGTCATTTCTGGAAA |
| | (TGFB2), RefSeqGene on chromosome 1 | GGGAAATGAGGGGGT |
| | | TTGTTCTCTTCATTC |
| SEQ ID | GI 493799665 NM_002994 Homo sapiens | CTGCGTTGCGTTTGT |
| NO: 884 | C-X-C motif chemokine ligand 5 | TTACAGACCACGCAA |
| | (CXCL5), mRNA | GGAGTTCATCCCAAA |
| | | ATGATCAGTAATCTG |
| SEQ ID | GI 119703747 NM_001432 Homo sapiens | CTCCTTCATCGAATG |
| NO: 885 | epiregulin (EREG), mRNA | CTAAACCTTTGAGTA |
| | | GAGTCTCCCTGGATC |
| | | ACATACCAGGTCAGG |
| SEQ ID | GI 1002341790 NM_000043 Homo sapiens | AAGTGTTAATGCCCA |
| NO: 886 | Fas cell surface death receptor (FAS), | AGTGACTGACATCAA |
| | transcript variant 1, mRNA | CTCCAAGGGATTGGA |
| | | ATTGAGGAAGACTGT |
| SEQ ID | GI 4455060 AF118265 Homo sapiens | AGTTAAAGCAGTGCT |
| NO: 887 | orphan G protein-coupled receptor | TCTCAAACCTTGATG |
| | GPR44 (GPR44) gene, complete cds | TGCCTGTGAATCACC |
| | | TAGGGGTCTTGTTAA |
| SEQ ID | GI 457232 L26524 Homo sapiens | ATTACCCTCCTCTTA |
| NO: 888 | PRKDC gene, complete sequence | CTTGGTGCTGTCTTA |
| | | CCTCATCAGTGAATG |
| | | ATGTCACCGTTCAGT |
| SEQ ID | GI 292658840 NG_016861 Homo sapiens | CCAGACACACATTAC |
| NO: 889 | C-X-C motif chemokine ligand 12 | CAAAATCTGGCAGGG |
| | (CXCL12), RefSeqGene on | TAGTAAAGTGCCTTG |
| | chromosome 10 | TTGTCCTTACCTCTT |
| SEQ ID | GI 189217843 NG_007930 Homo sapiens | TTTCTGGCCCAATTT |
| NO: 890 | cytochrome P450 family 2 subfamily | CTACCTACATTCCTC |
| | F member 1 (CYP2F1), RefSeqGene on | ACCCTAATTCCACTT |
| | chromosome 19 | GCCAAATCCTCTCAC |
| SEQ ID | GI 209977070 NG_008850 Homo sapiens | TGTTGAATACAGCAC |
| NO: 891 | interleukin l alpha (ILIA), RefSeqGene | CTACTGAAAGAACTT |
| | on chromosome 2 | TCATTCCCTGACCAT |
| | | GAACAGCCTCTCAGC |
| SEQ ID | GI 269954730 NG_016009 Homo sapiens | GTGCAACAGTGAGTG |
| NO: 892 | kininogen 1 (KNG1), RefSeqGene | AATGCAACCAATGTG |
| | on chromosome 3 | ACTGGAAGAAGTTGA |
| | | CTAGGTAGAGGGATC |
| SEQ ID | GI 291219867 NG_016781 Homo sapiens | TTCTAATGGTTGAGC |
| NO: 893 | pyruvate dehydrogenase alpha 1 | CTCAGAGTACATATT |
| | (PDHA1), RefSeqGene on chromosome X | TGGGGTGCGGTTTGG |
| | | TTTGCTTTGTAGAGT |
| SEQ ID | GI 401709987 NG_032900 Homo sapiens | AGTCCTTGGGGTTAA |
| NO: 894 | prostaglandin-endoperoxide synthase 1 | GTCTCCTGTCTTATG |
| | (PTGS1), RefSeqGene on chromosome 9 | GTCCAGAAACTCCTG |
| | | TTTCTCCTTAGTTGG |
| SEQ ID | GI 323276689 NG_028137 Homo sapiens | GAAGCTGTGAGGGAA |
| NO: 895 | secretory leukocyte peptidase | GATCTAGATATGAGG |
| | inhibitor (SLPI), RefSeqGene on | CCTCCTTCCTATGAA |
| | chromosome 20 | TACGTAGTGGGAGGA |
| SEQ ID | GI 1024846748 NG_046980 Homo sapiens | TTGGGGAAAACTTAG |
| NO: 896 | synaptonemal complex protein 2 (SYCP2), | AATACTGTCCCTTTC |
| | RefSeqGene on chromosome 20 | CTCCTGATCCTGAGG |
| | | TTTTGAGGTAAGGGA |
| SEQ ID | GI 695917236 NM_021010 Homo sapiens | GTCGCTGAGCTTCCT |
| NO: 897 | G | AGATAGAAACCAAAG |
| | | CAGTGCAAGATTCAG |
| | | TTCAAGGTCCTGAAA |
| SEQ ID | GI 14488405 AF386077 Homo sapiens | CTTCCATGATCTTCA |
| NO: 898 | interleukin 17B (IL17B) gene, | GTTTCCTCACGTGGA |
| | complete cds | ACATGGAGTTGATTA |
| | | CTGCCTTTCCTCCAG |
| SEQ ID | GI 368711279 NG_031983 Homo sapiens | CCAGAGTTCTTAACA |
| NO: 899 | heat shock protein family B (small) | CACATAAACTGCCTG |
| | member 6 (HSPB6), RefSeqGene on | GTCCTAAAAGAGTGC |
| | chromosome 19 | TGCAGAAACAGCTCT |
| SEQ ID | GI 237858686 NG_012087 Homo sapiens | GGGTCAATTCTCAAA |
| NO: 900 | interleukin 6 receptor (IL6R), | GGAAATGACATCACC |
| | RefSeqGene on chromosome 1 | TCATCTGAGATCCAA |
| | | AGGCCAAGTAGGGAC |
| SEQ ID | GI 1024846744 NG_047021 Homo sapiens | GATAAAACCATACGG |
| NO: 901 | nudix hydrolase 15 (NUDT15), | ATGTCAAAACTGTCT |
| | RefSeqGene on chromosome 13 | TCTTGTGCTGAGTCA |
| | | GTTCCTCGATGGGGG |
| SEQ ID | GI 300388175 NG_011471 Homo sapiens | TCGATCTGACCAGTT |
| NO: 902 | purinergic receptor P2X 7 (P2RX7), | TAGCAACCTCAGTGA |
| | RefSeqGene on chromosome 12 | AAAGAAAACACCTCT |
| | | TTGCCAGAAGGTTCA |
| SEQ ID | GI 238637280 NG_012233 Homo sapiens | CTGCATGTTACTGCC |
| NO: 903 | pyruvate dehyrogenase phosphatase | ACCACAAACATCTCT |
| | catalytic subunit 1 (PDP1), | GTTGTTCCTCATCGT |
| | RefSeqGene on chromosom [symbol] | ACATTCCTCAGAGTC |
| SEQ ID | GI 238018083 NG_012204 Homo sapiens | GTCTCAGGAAGTGGT |
| NO: 904 | peroxisome proliferator activated | TTAGGATGTTTCAGC |
| | receptor alpha (PPARA), | GAGAGCATGATACAG |
| | RefSeqGene on chromosome 22 | ACTAACCCAGGAAGA |
| SEQ ID | GI 341572562 NG_029524 Homo sapiens | CAACCTTTAGGATGG |
| NO: 905 | TNF superfamily member 13b (TNFSF13B), | ACAGGTTGCCCTTGT |
| | RefSeqGene on chromosome 13 | GATGTCAGCAAACCC |
| | | TTGACCAGTATATAA |
| SEQ ID | GI 126116577 NM_176823 Homo sapiens | GGTCGTCAAGAGAAC |
| NO: 906 | S100 calcium binding protein A7A | TGTGTCATTCCAAAC |
| | (S100A7A), mRNA | TCACCAAGGTGGCTT |
| | | ATAGAACAGAAGCAG |
| SEQ ID | GI 225579056 NM_003049 Homo sapiens | GTAAACTAGAGAGAG |
| NO: 907 | solute carrier family 10 member 1 | CAGCAAAAACACCAG |
| | (SLC10A1), mRNA | TCTTGCCTGAGTCTT |
| | | TCTCCAGCATTTCCA |
| SEQ ID | GI 269954683 NG_015990 Homo sapiens | AAAATGAGCTAGAAC |
| NO: 908 | C-C motif chemokine ligand 5 (CCL5), | AGGGAAAGCTGAGAA |
| | RefSeqGene on chromosome 17 | AACAGGAGTCCAGTT |
| | | TAGCGGCTACTGTTG |
| SEQ ID | GI 973353146 NG_016274 Homo sapiens | ATTCTTTCCCAAAGG |
| NO: 909 | hepatocyte growth factor (HGF), | TCCCTTCTTGAGATA |
| | RefSeqGene on chromosome 7 | CATGTGTTTCTCAGA |
| | | AGCCAGGCATGTCAA |
| SEQ ID | GI 193211405 NG_008118 Homo sapiens | GAGTGGGAAGACCTG |
| NO: 910 | left-right determination factor 2 | ACTTTATGGGGGTAT |
| | (LEFTY2), RefSeqGene on chromosome 1 | TGATTGTGGATCATC |
| | | GACTGTGTGTGTTTG |
| SEQ ID | GI 236459493 NG_012010 Homo sapiens | ATAGCTCATACACAC |
| NO: 911 | lymphotoxin alpha (LTA), RefSeqGene | CCAGAATGTTACTTA |
| | on chromosome 6 | CATGTCCCTCCATGT |
| | | TACACCAAGACCCCT |
| SEQ ID | GI 340007392 NG_029438 Homo sapiens | CAGGGAGGTGACGGG |
| NO: 912 | mitogen-activated protein kinase | TCTGTCATGCTTCTA |
| | kinase 7 (MAP2K7), RefSeqGene on | TCCCTGTTTCACCCT |
| | chromosome 19 | CTTTATTTTTTTTGA |
| SEQ ID | GI 291045457 NG_016717 Homo sapiens | GGCGTTAACAAGCAA |
| NO: 913 | phosphoribosyl pyrophosphate | TCAGGGTACAGGGTG |
| | synthetase 2 (PRPS2), RefSeqGene | TTCAGAATCAAGGAT |
| | on chromosome X | GAGCACAATAAGAGA |
| SEQ ID | GI 341823644 NG_029509 Homo sapiens | TCCAAGACCCAGGAG |
| NO: 914 | prostaglandin E receptor 3 (PTGER3), | CAGACTATAATTGAA |
| | RefSeqGene on chromosome 1 | GCCAGTTGTCTAATC |
| | | CCACTGAGAATCACA |
| SEQ ID | GI 209447083 NG_008732 Homo sapiens | CATTTGTTTGTACAA |
| NO: 915 | vascular endothelial growth factor A | GATCCGCAGACGTGT |
| | (VEGFA), RefSeqGene on chromosome 6 | AAATGTTCCTGCAAA |
| | | AACACAGACTCGCGT |
| SEQ ID | GI 406737 X75342 H. sapiens SHB mRNA | GAGGAGCAACCAGGG |
| NO: 916 | | TTTTATGCACATGAA |
| | | ACTGGCCAAAACCAA |
| | | AGAGAAATACGTTCT |
| SEQ ID | GI 374349244 NG_032042 Homo sapiens | CCCTCTTGAATGAGT |
| NO: 917 | annexin A5 (ANXA5), RefSeqGene on | CATTTAAAGTCACAT |
| | chromosome 4 | TTGCAGACAGTTTTG |
| | | GGTGGGCAGTGATGG |
| SEQ ID | GI 1047225988 NG_051241 Homo sapiens | TTGAATCAAGTTGAT |
| NO: 918 | arrestin domain containing 3 (ARRDC3), | GTGGGTTCCGAACTG |
| | RefSeqGene on chromosome 5 | TATCTCTTCCGGCTG |
| | | AGGACAGAGAAGTAT |
| SEQ ID | GI 927669118 NM_001166693 Homo sapiens | CCAAACTGAAGATGC |
| NO: 919 | AF4/FMR2 family member 1 (AFF1), | CTTCTCAGTCAGTTG |
| | transcript variant 1, mRNA | AGCAGACCTACTCCA |
| | | ATGAAGTCCATTGTG |
| SEQ ID | GI 14278713 AF377330 Homo sapiens | CAGCACTGTCTCAGT |
| NO: 920 | urokinase-type plasminogen activator | TTCACTTTCACATAG |
| | (PLAU) gene, complete cds | ATGTCCCTTTCTTGG |
| | | CCAGTTATCCCTTCC |
| SEQ ID | GI 306518675 NG_027688 Homo sapiens | TCTCAGAGCATCCAG |
| NO: 921 | CD4 molecule (CD4), RefSeqGene on | TTAGGGGTGTGCAAG |
| | chromosome 12 | TTTACTTTGTTACTG |
| | | GCTGATGTTGTGAAG |
| SEQ ID | GI 1013071127 NG_021208 Homo sapiens | TTCTTTCCATTCTGC |
| NO: 922 | opioid receptor mu1 (OPRM1), | ATCACAGCACAGAGC |
| | RefSeqGene on chromosome 6 | CATCCCATCTGCTCA |
| | | CATGATTAGAATCAT |
| SEQ ID | GI 908212430 NM_000412 Homo sapiens | GGTCCTCATAGATTT |
| NO: 923 | histidine rich glycoprotein (HRG), | CTTTGAGGATACTGA |
| | mRNA | GCGCTACAGAAAACA |
| | | AGCCAACAAAGCCCT |
| SEQ ID | GI 1060856491 NM_005526 Homo sapiens | TCCTTGAGAACATCA |
| NO: 924 | heat shock transcription factor 1 | AGAGGAAAGTGACCA |
| | (HSF1), mRNA | GTGTGTCCACCCTGA |
| | | AGAGTGAAGACATAA |
| SEQ ID | GI 1159261125 NM_001349413 Homo sapiens | AGAGTTTGACCTCAT |
| NO: 925 | kelch like family member 24 (KLHL24), | TAGTATGGTGCTTTG |
| | transcript variant 1, mRNA | GGTGAGGACCTCTTC |
| | | CTTGATTATCCCACT |
| SEQ ID | GI 543583730 NM_006601 Homo sapiens | GCATGAACATTTCCC |
| NO: 926 | prostaglandin E synthase 3 (PTGES3), | AGACTTTCATGAAGC |
| | transcript variant 1, mRNA | TGTTGAGGTCCTAGG |
| | | CAATTAATGCAGCAG |
| SEQ ID | GI 41324131 AY527817 Homo sapiens | AGAAAAATATTTCTC |
| NO: 927 | arachidonate 12-lipoxygenase (ALOX12) | AGGCCCCAAGCCCAA |
| | gene, complete cds | AGCTGTGCTAAACCA |
| | | ATTCCGAACAGATTT |
| SEQ ID | GI 67515420 DQ088846 Homo sapiens | GGCAAAGCTGAAATT |
| NO: 928 | myeloperoxidase (MPO) gene, complete | TGAAGCCAGATTAGC |
| | cds | CTAACCTCAAAGCCT |
| | | GTGCCTATGTTATGG |
| SEQ ID | GI 1036032775 AH003000 Homo sapiens | GCCACTGTTGGCATG |
| NO: 929 | steroid 5-alpha-reductase (SRD5A1) | TACAATGGCGATTAT |
| | gene, complete cds | GTTCTGTACCTGTAA |
| | | CGGCTATTTGCAAAG |
| SEQ ID | GI 570359607 NG_033871 Homo sapiens | TTGTACTACTTCCAC |
| NO: 930 | F-box protein 38 (FBX038), | CACTACATCATTCTT |
| | RefSeqGene on chromosome 5 | GAACATGACAGCTGG |
| | | TGCCTAGCATTGCAC |
| SEQ ID | GI 307133762 NG_027719 Homo sapiens | AGGGCTTTTAGGGAG |
| NO: 931 | glutathione-disulfide reductase | AGTGACTCATAGTCC |
| | (GSR), RefSeqGene on chromosome 8 | ATGAGCCTATCTTCA |
| | | CTTTGATTCACCCAG |
| SEQ ID | GI 171460912 NG_007496 Homo sapiens | GATCCAGACAGGACT |
| NO: 932 | jagged 1 (JAG1), RefSeqGene on | AGGTCAGCCCAGTTG |
| | chromosome 20 | TTGTCTGAACAGGCT |
| | | ATCCTATAGGAAATG |
| SEQ ID | GI 48772828 AY618665 Homo sapiens | AAACGACAAGAAGCT |
| NO: 933 | VIMP mRNA, complete cds | TTAGCAGCTGCTCGA |
| | | CTGAAAATGCAAGAA |
| | | GAACTAAATGCGCAA |
| SEQ ID | GI 210032246 NG_008862 Homo sapiens | ACAAAGGGAGGGGAA |
| NO: 934 | phosphoglycerate kinase 1 | TGGTTATCAGAATTC |
| | (PGK1), RefSeqGene on chromosome X | AGAGAGGAAAGTTGG |
| | | TGTGGAGAGAGCTAT |
| SEQ ID | GI 205277388 NG_008604 Homo sapiens | CCTTGGATGACAGGC |
| NO: 935 | polycystin 2, transient receptor | ACCATAACCCTTAAC |
| | potential cation channel (PKD2), | CCACTTGGTAATCTC |
| | RefSeqGene on chromoson | TCATTTTTGGTCTGT |
| SEQ ID | GI 156104863 NM_003501 Homo sapiens | GTCTGAAATCGAAGC |
| NO: 936 | acyl-CoA oxidase 3, pristanoyl (ACOX3), | TCTAGTGGGACTGGC |
| | transcript variant 1, mRNA | ACACATTCAGCCAAG |
| | | TCTAATGAAACGAAG |
| SEQ ID | GI 66933013 NM_020139 Homo sapiens | GGTTTTGGACCGGGC |
| NO: 937 | 3-hydroxybutyrate dehydrogenase 2 | ACTACACTACCGAAT |
| | (BDH2), mRNA | TATGGCTAACTTTGG |
| | | TTTGTGATCTAGACC |
| SEQ ID | GI 392996937 NM_001008387 Homo sapiens | CTGGAAGAGAATATT |
| NO: 938 | regenerating family member 3 gamma | CTCCCCAAACTGCCC |
| | (REG3G), transcript variant 1, mRNA | TACCTGACTACCTTG |
| | | TCATGATCCTCCTTC |
| SEQ ID | GI 1049010967 AH003492 Homo sapiens | GGCCCTTCTTCCCAC |
| NO: 939 | beta-fibrinogen (FGB) gene, complete | AGCAATAGTCCCCAA |
| | cds | TACGTAGATTTTTGC |
| | | TCTTCTGTATGTGAC |
| SEQ ID | GI 226530635 NG_011844 Homo sapiens | GAGTCCCTGCTTCAG |
| NO: 940 | adenosine deaminase, RNA specific | AATCTTATTCCTGAA |
| | (ADAR), RefSeqGene on chromosome 1 | AGGTTTCCATCTTTC |
| | | TCCCGTTGCTTCTGG |
| SEQ ID | GI 190358505 NG_008040 Homo sapiens | GAAACCAGAAATCAG |
| NO: 941 | colony stimulating factor 2 receptor | GTGCCGTGTCTTCAT |
| | beta common subunit (CSF2RB), | TCTGCATTTTCTTGA |
| | RefSeqGene on chrome | ACAACCCAGAGTTCC |
| SEQ ID | GI 261823945 NG_013238 Homo sapiens | AGTCAGTAGACAAAT |
| NO: 942 | interleukin 9 receptor (IL9R), | CACTGAAGTCCATGT |
| | RefSeqGene on chromosome X and Y | CTGGCAGTTCTCAAT |
| | | GTCATGGGGACTGTA |
| SEQ ID | GI 209413721 NM_021068 Homo sapiens | GCAGTGTGAAGAAGC |
| NO: 943 | interferon alpha 4 (IFNA4), mRNA | TTGGTGTATACCTGT |
| | | GCAGGCACTAGTCCT |
| | | TTACAGATGACAATG |
| SEQ ID | GI 264681561 NM_018441 Homo sapiens | GTTCCTGGGGACAAA |
| NO: 944 | peroxisomal trans-2-enoyl-CoA | GCTTCTTTGAAGGGT |
| | reductase (PECR), mRNA | CTTTTCAGAAAATCC |
| | | CCGCTAAACGAATTG |
| SEQ ID | GI 295842487 NM_014470 Homo sapiens | CTCCCTACTACGATA |
| NO: 945 | Rho family GTPase 1 (RND1), mRNA | ATGTCCGTCCACTCT |
| | | GCTACAGCGACTCGG |
| | | ATGCAGTATTACTAT |
| SEQ ID | GI 32263626 AY325910 Homo sapiens | ATCTAAGTCATATGG |
| NO: 946 | hydroxysteroid (17-beta) | GGAAGGGAGTTCTTT |
| | dehydrogenase 2 (HSD17B2) gene, | GCAGTGAGCTGTTTC |
| | complete cds | CCAGAACACAAAAGG |
| SEQ ID | GI 13447389 AF361105 Homo sapiens | TCCTGGTGGGAGAGG |
| NO: 947 | interleukin 9 (IL9) gene, | CAGATAAAAGATCTG |
| | complete cds | CAGAAAAACAGGAGT |
| | | TCAGGAGGATTAAGA |
| SEQ ID | GI 51988875 AY744675 Homo sapiens | GCAACAGTAAGTCAC |
| NO: 948 | matrix metalloproteinase 10 | AAGTTGACTTGGCTG |
| | (stromelysin 2) (MMP10) gene, | GCTCAGTGTATATCC |
| | complete cds | CAGTTTCTCTCCAAC |
| SEQ ID | GI 226530635 NG_011844 Homo sapiens | TTCTGCCTTTTAGCT |
| NO: 949 | adenosine deaminase, RNA specific | AAATTCCCCTTGATT |
| | (ADAR), RefSeqGene on chromosome 1 | GGCCCTTTCTCCAGA |
| | | TATTGAGCAGGGAAT |
| SEQ ID | GI 261598995 NG_013210 Homo sapiens | TACACACACAGACTG |
| NO: 950 | aspartate beta-hydroxylase (ASPH), | AAATTGTGGTAAGAG |
| | RefSeqGene on chromosome 8 | TTCTGAGGAAACAAA |
| | | GCAGGAGAGCATGCC |
| SEQ ID | GI 237874181 NG_012163 Homo sapiens | TGCAGGGTAAGAGGT |
| NO: 951 | HtrA serine peptidase 2 (HTRA2), | TGTACCACATCTTGG |
| | RefSeqGene on chromosome 2 | ATAGAAAATGGCCAG |
| | | GAACTAGAAGAGGAA |
| SEQ ID | GI 429535869 NG_033019 Homo sapiens | TTTGGAAAACTGTTC |
| NO: 952 | interleukin 5 (IL5), RefSeqGene on | TGGGCAACAGTTTGT |
| | chromosome 5 | GTTTGGTACCATCTG |
| | | TTCTTGACGCTCAAG |
| SEQ ID | GI 307133693 NG_027728 Homo sapiens | TTGAATGGAGTCATT |
| NO: 953 | interferon regulatory factor 4 | TTGGCTTCTTCACTG |
| | (IRF4), RefSeqGene on chromosome 6 | GCACCTCTCTGAATT |
| | | TCTAGGAATGTGCCT |
| SEQ ID | GI 209977032 NG_008779 Homo sapiens | CAGGAGCAAGCCAAA |
| NO: 954 | paraoxonase 1 (PON1), RefSeqGene on | TCCCCAGGATAGATG |
| | chromosome 7 | CATCCTTTTTTCTTC |
| | | AGACTTGGAGTGTAT |
| SEQ ID | GI 29542 X06596 Human mRNA for | ACATAGAAGTTCCTG |
| NO: 955 | complement component Cls | AAGGGTATGGGATTC |
| | | ACCTCTACTTCACCC |
| | | ATCTGGACATTGAGC |
| SEQ ID | GI 126116577 NM_176823 Homo sapiens | CACACTCACACCTCT |
| NO: 956 | S100 calcium binding protein A7A | ATTCACAGACCAGCA |
| | (S100A7A), mRNA | TCTCCTCTCCTTATC |
| | | AGGAACATTCCTTCC |
| SEQ ID | GI 915408 U30245 Human myelomonocytic | CAGTACAAAGGGTTT |
| NO: 957 | specific protein (MNDA) gene, | ATCAGGCCCCCTATC |
| | 5′ flanking sequence and complete | ATTCCAGAGTTTCAG |
| | exon 1 | TGCTGTTCCAGAATG |
| SEQ ID | GI 310703611 NG_027845 Homo sapiens | TCCATTGGTAATGTC |
| NO: 958 | clusterin (CLU), RefSeqGene on | TGGCATGAGTAGAGA |
| | chromosome 8 | GAGTGGAGTCATCAA |
| | | AAGGATGTAGGCCAG |
| SEQ ID | GI 259013222 NG_012922 Homo sapiens | AATCAGTGCTTATTT |
| NO: 959 | glycogen synthase kinase 3 beta | TAGGAAACAGGTTGC |
| | (GSK3B), RefSeqGene on chromosome 3 | CCCCCACAACTGGGG |
| | | TAAAAGAAGAGAGAA |
| SEQ ID | GI 257467500 NG_012841 Homo sapiens | GATGGAAGGACACCT |
| NO: 960 | interleukin 13 receptor subunit | GACTTTGGAAAGAGG |
| | alpha 1 (IL13RA1), RefSeqGene | GACATCATTCTTTCC |
| | on chromosome X | TGCCAGACATTTTCT |
| SEQ ID | GI 225543321 NG_011651 Homo sapiens | GCTATGAGGATTGCA |
| NO: 961 | NLRfamily pyrin domain containing 1 | GCAGTGTTTTTCTAT |
| | (NLRP1), RefSeqGene on chromosome 17 | TCTTCTCCTGTTCCC |
| | | ACGAGCTGTGATCTG |
| SEQ ID | GI 354459375 NG_030392 Homo sapiens | AAATACTGCCCACAG |
| NO: 962 | nuclear receptor subfamily 1 group H | GTTTTGGTCTTCTCC |
| | member 3 (NR1H3), RefSeqGene on | CATTTTCCTCTCCTT |
| | chromosome 11 | TTCCCTCTTTGTAGG |
| SEQ ID | GI 20514390 AF507942 Homo sapiens | AAGCGGCACTAGGGA |
| NO: 963 | cyclin-dependent kinase 4 (CDK4) | AAAAGAGGGAAGCAC |
| | gene, complete cds | AATAACTCCAAATGA |
| | | GTAGTCACAAGGTTT |
| SEQ ID | GI 21885466 AY128534 Homo sapiens | CATGGAACCTGGAGA |
| NO: 964 | cyclin-dependent kinase 6 (CDK6) | GGGGAGTTAGCTGAT |
| | gene, complete cds | GAGATTAATGTCTGA |
| | | TAGCCTCAAGCTCTG |
| SEQ ID | GI 85725379 DQ370420 Homo sapiens | CAACGGATACGTCAT |
| NO: 965 | metallothionein 2A (MT2A) gene, | CAGTAACCACCCCAC |
| | complete cds | GGGTTTACTGTGATG |
| | | CTGCACAATTATTAA |
| SEQ ID | GI 297139781 NG_021374 Homo sapiens | CTAAAATCAGAGGAA |
| NO: 966 | early growth response 1 (EGR1), | CAATGAGGTATCCCC |
| | RefSeqGene on chromosome 5 | TTGACCTGGTATGGT |
| | | TATCAGGCCCTGAGT |
| SEQ ID | GI 539847481 NM_003837 Homo sapiens | ACTGGATGTGCTATC |
| NO: 967 | fructose-bisphosphatase 2 (FBP2), | CAATTCCCTGGTGAT |
| | mRNA | CAACATGGTCCAATC |
| | | CTCCTATAGTACCTG |
| SEQ ID | GI 166706902 NM_002053 Homo sapiens | TCTTGGGAAATATTT |
| NO: 968 | guanylate binding protein 1 (GBP1), | GGGCATTGGTCTGGC |
| | mRNA | CAAGTCTACAATGTC |
| | | CCAATATCAAGGACA |
| SEQ ID | GI 164696344 AK313967 Homo sapiens | TGAAACATTGCTCGT |
| NO: 969 | cDNA, FU94614, Homo sapiens | GTTCACTTGCACATG |
| | interleukin 18 receptor 1 (IL18R1), | AGATTGAAACAACCA |
| | mRNA | CCAAAAGCTGGTACA |
| SEQ ID | GI 221136810 NM_006931 Homo sapiens | TGAAGACTTGAATTA |
| NO: 970 | solute carrier family 2 member 3 | GATTACAGCGATGGG |
| | (SLC2A3), mRNA | GACACAGAAGGTCAC |
| | | CCCAGCTCTGATATT |
| SEQ ID | GI 262205923 NM_013444 Homo sapiens | GATTCAGAGAAACCC |
| NO: 971 | ubiquilin 2 (UBQLN2), mRNA | AGAAATCAGTCACCT |
| | | GCTCAACAACCCAGA |
| | | CATAATGAGGCAGAC |
| SEQ ID | GI 1059792660 AH007003 Homo sapiens | AGAGAAGAGGCCAGA |
| NO: 972 | MET proto-oncogene, intron 2, | AATGGGAGTTTCTCA |
| | 3′ end | ACCATTTATGCGACA |
| | | AGTCTTCAGGTGTTG |
| SEQ ID | GI 335892864 NG_029105 Homo sapiens | CCTCTGCTAGCCGTC |
| NO: 973 | interleukin 1 receptor accessory | TTCCTCTGCTGATGA |
| | protein (ILIRAP), RefSeqGene on | GTGATTTCTGTTTGA |
| | chromosome 3 | TGCAAACTTTAGCTT |
| SEQ ID | GI 645912997 NG_034139 Homo sapiens | GTGCTTCGAGGTTTT |
| NO: 974 | MHC class 1 polypeptide-related | CTGATTTTGAAGAAT |
| | sequence A (MICA), RefSeqGene | CCCAGTCAGTCAGGT |
| | on chromosome 6 | TCTGGCGTAAAGTGA |
| SEQ ID | GI 110349787 NM_018489 Homo sapiens | GCTCACTCCCAAAAA |
| NO: 975 | ASH1 like histone lysine | AGATTTCTCGCCTCA |
| | methyltransferase (ASHIL), mRNA | TTACGTCCCAGACAA |
| | | CTACAAGAGGAATGG |
| SEQ ID | GI 1002341790 NM_000043 Homo sapiens | GAGTATTACTAGAGC |
| NO: 976 | Fas cell surface death receptor | TTTGCCACCTCTCCA |
| | (FAS), transcript variant 1, mRNA | TTTTTGCCTTGGTGC |
| | | TCATCTTAATGGCCT |
| SEQ ID | GI 193290109 NG_008150 Homo sapiens | AGCTTTTATTCATGC |
| NO: 977 | checkpoint kinase 2 (CHEK2), | TACTTGTCAGTCTCC |
| | RefSeqGene on chromosome 22 | CTCAGGGTTCCTTAG |
| | | GCTGTCCTTGAGTAC |
| SEQ ID | GI 1025608661 NG_047049 Homo sapiens | GGAGGAGCTGAGAGT |
| NO: 978 | fatty acid binding protein 3 | TGGCAAATCAATAGT |
| | (FABP3), RefSeqGene on chromosome 1 | TTAGCTATGTTGCTG |
| | | AACCTTCCTGGATGG |
| SEQ ID | GI 231570519 NG_012004 Homo sapiens | AAGGTCAATGGGACG |
| NO: 979 | kinase insert domain receptor | ATCACTTCAGTGAGC |
| | (KDR), RefSeqGene on chromosome 4 | AGATAGCTCTGTAAA |
| | | TTCCTCCATAGAGGC |
| SEQ ID | GI 1049010846 AH002874 Homo sapiens | CACATTTGCCTTTGA |
| NO: 980 | MC-CPA gene, complete sequence; | GCTCCGAGATAAAGG |
| | and carboxypeptidase A (CPA3) | CAAATTTGGTTTTCT |
| | gene, complete cds | CCTTCCAGAATCCCG |
| SEQ ID | GI 67515420 DQ088846 Homo sapiens | ATTCTGAGGTTGCAT |
| NO: 981 | myeloperoxidase (MPO) gene, | CCAATTTTAGCAGGA |
| | complete cds | AAGAGTGCTGTGATC |
| | | GTTAGCTGCATCAGA |
| SEQ ID | GI 1147489694 NG_016780 Homo sapiens | GCTAACAGGATTAGC |
| NO: 982 | acyl-CoA thioesterase 9 (ACOT9), | CCAGGTGCTGGTGGA |
| | RefSeqGene on chromosome X | AGAAAGGAGGAACTA |
| | | TTGAAAGGTTTGAAT |
| SEQ ID | GI 1031698869 NG_050687 Homo sapiens | GCATGAATTCAAGTA |
| NO: 983 | HOP homeobox (HOPX), RefSeqGene on | TAGCTGTCATCTCAG |
| | chromosome 4 | TCTTCCTGCCTCATC |
| | | AAGTCAAGTGCAGCG |
| SEQ ID | GI 240255451 NG_012330 Homo sapiens | CTACAGCCCTCTGGT |
| NO: 984 | MYB proto-oncogene, transcription | CAGCAGCACCAACTT |
| | factor (MYB), RefSeqGene on | TCTCACGGAGTTAAA |
| | chromosome 6 | TTAGTTTGGGTTATG |
| SEQ ID | GI 291084846 NG_016762 Homo sapiens | GAGGAGTCATGTGAA |
| NO: 985 | pyruvate dehydrogenase kinase 3 | GGAAAGCTACCTTTA |
| | (PDK3), RefSeqGene on chromosome X | GTCCTCCATCGCAAT |
| | | ACTGTCCAGTGTCTC |
| SEQ ID | GI 206725542 NG_008631 Homo sapiens | CTTCAGACAGTGCCA |
| NO: 986 | patatin like phospholipase domain | GTCCTTTTTCCCCTT |
| | containing 3 (PNPLA3), RefSeqGene | CTTTAAAAGCGATAT |
| | on chromosome 22 | GTGGATGGAGGAGTG |
| SEQ ID | GI 238018078 NG_012202 Homo sapiens | GACATTGAAAAAACA |
| NO: 987 | TNF receptor superfamily member | ATAAAGGCATGCAAA |
| | 11b (TNFRSF11B), RefSeqGene on | CCCAGTGACCAGATC |
| | chromosome 8 | CTGAAGCTGCTCAGT |
| SEQ ID | GI 219521851 NG_009233 Homo sapiens | TTTTTATGTCAGTGC |
| NO: 988 | tyrosine 3-monooxygenase/tryptophan | CTGATGCTGTGCCCT |
| | 5-monooxygenase activation protein | TTTCTTCCCTTGGTG |
| | epsilon (YWHA | TGATTGCTTTTACTG |
| SEQ ID | GI 45243506 NM_002935 Homo sapiens | AGTATACGCTGCCCT |
| NO: 989 | ribonuclease A family member 3 | CATAACAGAACTCTC |
| | (RNASE3), mRNA | AACAATTGTCATCGG |
| | | AGTAGATTCCGGGTG |
| SEQ ID | GI 27369416 AY166858 Homo sapiens | AAGTTCTCAGTAATG |
| NO: 990 | TGS1 mRNA, complete cds | AACCTCATGAGACAA |
| | | TGCCAATTGCTGCCT |
| | | TGGGACAAAAACAGC |
| SEQ ID | GI 1036032792 AH002827 Homo sapiens | GAGAGTACTTTTGAA |
| NO: 991 | hemopexin (HPX) gene, complete cds | TGCAGGGGGACAAAG |
| | | TCTGGGTATACCCTC |
| | | CTGAAAAGAAGGAGA |
| SEQ ID | GI 82583661 DQ297144 Homo sapiens | AGTTTGATCTCAAAA |
| NO: 992 | metallothionein 3 (growth inhibitory | TCTCCCCAGCTCACG |
| | factor (neurotrophic)) (MT3) gene, | GCAGTGTAATATGCA |
| | complete cds | TAGGGAGTAAGAGGT |
| SEQ ID | GI 1064244211 NG_051562 Homo sapiens | AAGCTACCAACAAAG |
| NO: 993 | ChaC glutathione specific gamma- | GAGGTGCTCATATGA |
| | glutamylcyclotransferase 1 (CHAC1), | ACGAAAGGGGACAGG |
| | RefSeqGene on chr [symbol] | ATAGAAGAGAAGAGG |
| SEQ ID | GI 238018116 NG_012218 Homo sapiens | TCTCACACCAGGATG |
| NO: 994 | hexose-6-phosphate dehydrogenase/glucose | ACTGGGTCTTAGCAC |
| | 1-dehydrogenase (H6PD), RefSeqGene | GACCTAATCGTCTTC |
| | on chro [symbol] | ATGATGGAAAGAAAG |
| SEQ ID | GI 254911080 NG_012514 Homo sapiens | CTTGTGAAAAACTCC |
| NO: 995 | interleukin 13 receptor subunit alpha 2 | GGTGTTCCTGGTAAC |
| | (IL13RA2), RefSeqGene on chromosome X | CACATAGTGTATAAA |
| | | GCCCCTCATTGCACT |
| SEQ ID | GI 908212454 NG_042284 Homo sapiens | TTTGGGGTTCCTCAG |
| NO: 996 | SMAD family member 1 (SMAD1), | CGCTATGAGGCTACT |
| | RefSeqGene on chromosome 4 | AGAAGCTCTGCTTAG |
| | | TTTTTAGCCTCTTAG |
| SEQ ID | GI 189458878 NM_139166 Homo sapiens | CCTGTCTTAGTACCA |
| NO: 997 | actin binding Rho activating protein | ACAAAGGAAGGCACA |
| | (ABRA), mRNA | CCCTCACTAAAAGTA |
| | | GAGCAGAGGTGAGAG |
| SEQ ID | GI 164695701 AK315511 Homo sapiens | GTGATGGTTTCTCGA |
| NO: 998 | cDNA, FLI96576, Homo sapiens | GCAGAAATTGACATG |
| | annexin A4 (ANXA4), mRNA | TTGGATATCCGGGCA |
| | | CACTTCAAGAGACTC |
| SEQ ID | GI 166795249 NM_006845 Homo sapiens | CAATAAACCCAGAAC |
| NO: 999 | kinesin family member 2C | TCTTACAGCTTCTTC |
| | (KIF2C), transcript variant 1, mRNA | CCTTACATCCGAAGG |
| | | ACAATCTGCCCTTGC |
| SEQ ID | GI 571330780 NM_001136022 Homo sapiens | CTTCAGCACCACCTC |
| NO: 1000 | nuclear factor of activated T-cells 4 | TAAGATCTCTGAGAG |
| | (NFATC4), transcript variant 1, mRNA | AGGGTGGATCAGCCT |
| | | CTGTGTAAACAAAAA |
| SEQ ID | GI 40288420 AY505111 Homo sapiens | GGCGATATGGAACAA |
| NO: 1001 | arachidonate 15-lipoxygenase (ALOX15) | CAGAGAGGAAATTCA |
| | gene, complete cds | TGGGATTAAGGGCAG |
| | | ATAGGCTTGGATGTG |
| SEQ ID | GI 1036032759 AH002916 Homo sapiens | CTTTATTGCTGATGA |
| NO: 1002 | ornithine aminotransferase (OAT) gene, | AATACAGACAGGATT |
| | complete cds | GGCCAGAACTGGTAG |
| | | ATGGCTGGCTGTTGA |
| SEQ ID | GI 223890186 NG_009797 Homo sapiens | AGAGATGCTTTTCCC |
| NO: 1003 | junctophilin 3 (JPH3), RefSeqGene on | GACGTGAATGGAATA |
| | chromosome 16 | TTCTGAGGATTAGGA |
| | | CGTGGACCTACCTTG |
| SEQ ID | GI 916539820 NG_042809 Homo sapiens | CTGGTAGTTCTGTAA |
| NO: 1004 | potassium voltage-gated channel | TTAAGCAAGTGACCA |
| | subfamily J member 12 (KCNJ12), | CCCCTCTGTGCCTCA |
| | RefSeqGene on chromos | GTTCCTTCATCTGTA |
| SEQ ID | GI 584613557 NG_033959 Homo sapiens | GAAAAAGATGCCTGT |
| NO: 1005 | poly(ADP-ribose) polymerase 2 (PARP2), | GGCTGGAGGAAAAGC |
| | RefSeqGene on chromosome 14 | TAATAAGGACAGGAC |
| | | AGAAGACAAGCAAGA |
| SEQ ID | GI 90657247 DQ449938 Homo sapiens | TCCTTCCCAGACCTG |
| NO: 1006 | Fas (TNFRSF6)-associated via death | TGTGCAGCATTTAAC |
| | domain (FADD) gene, complete cds | GTCATATGTGATAAT |
| | | GTGGGGAAAGATTGG |
| SEQ ID | GI 31559181 AY310878 Homo sapiens | GGGATCCTAGTAACT |
| NO: 1007 | glutathione peroxidase 3 (plasma) | GCTGGTTTCTAGGTG |
| | (GPX3) gene, complete cds | ACTAAACCTGGCAGA |
| | | GAGAATCATGGAACC |
| SEQ ID | GI 1024846757 NG_047018 Homo sapiens | TGTGCAAGTCACTTT |
| NO: 1008 | histone deacetylase 1 (HDAC1), | TCCCCTGTAGGCCTC |
| | RefSeqGene on chromosome 1 | GGTTTATTCTCCGTA |
| | | AAATCAGAAAGTTGG |
| SEQ ID | GI 342307067 NG_029605 Homo sapiens | TTACACCCAGCGTTT |
| NO: 1009 | interleukin 15 (IL15), RefSeqGene on | CTTTAATAGGAGTGG |
| | chromosome 4 | AGTAGGGTTGTTTAC |
| | | AAAGGCCCTGGTCTA |
| SEQ ID | GI 365777421 NG_031966 Homo sapiens | TATAAAGCCAATTCC |
| NO: 1010 | interleukin 21 (IL21), RefSeqGene on | CGCAGTTATTTATCC |
| | chromosome 4 | TGGACCACATATTGC |
| | | CAGACACAGCTCTGC |
| SEQ ID | GI 210032137 NG_008855 Homo sapiens | TTTCCAGGCTGTCTT |
| NO: 1011 | lipoprotein lipase (LPL), RefSeqGene | CTGAGTTGAACATGA |
| | on chromosome 8 | TGGCAAAGGAGAGCA |
| | | AAATAGCAGATGTCA |
| SEQ ID | GI 1140398720 NG_052834 Homo sapiens | ATTCCTTTGCTGCCT |
| NO: 1012 | TNF receptor superfamily member 9 | CTTATCTTCTCTCAT |
| | (TNFRSF9), RefSeqGene on chromosome 1 | TGATCAGCCTGTCTC |
| | | CATGTGTTCAATGCC |
| SEQ ID | GI 727346118 NM_002728 Homo sapiens | TTCTCCTCTGATCGC |
| NO: 1013 | proteoglycan 2, pro eosinophil major | GGATCCACTCTGCTT |
| | basic protein (PRG2), transcript | AGCCCTCATTGAAAC |
| | variant 1, mRNA | TTCTTCCTTATCATC |
| SEQ ID | GI 77744386 DQ230907 Homo sapiens | CCACTAGACTGAAAG |
| NO: 1014 | nudix (nucleoside diphosphate linked | AAGTTCCTGTTAATC |
| | moiety X)-type motif 1 (NUDT1) gene, | CAAAGGCCTGACCTG |
| | complete cds | AGGACCACAGGATAA |
| SEQ ID | ref \| NG_009824.2\| Homo sapiens | TATGGGCTCTACGAG |
| NO: 1015 | crystallin alpha B (CRYAB), | AATCTGTGAAACCCA |
| | RefSeqGene on chromosome | TCATTCTTGTGTTCA |
| | | GTTCTCTCACCCCAT |
| SEQ ID | GI 260763953 NG_013056 Homo sapiens | GATGAGGAAGAATAA |
| NO: 1016 | chitinase 3 like 1 (CHI3L1), | AGCAGTCAAAGAAGA |
| | RefSeqGene on chromosome 1 | GCAGTGTTAGGCTGC |
| | | AATTCTCCATCCGCA |
| SEQ ID | GI 260763953 NG_013056 Homo sapiens | GGAACGTAATCGTGT |
| NO: 1017 | chitinase 3 like 1 (CHI3L1), | CCCCTATCCTACTTC |
| | RefSeqGene on chromosome 1 | CCCTTCCTAATTCCA |
| | | CAGCTGCTCAATAAA |
| SEQ ID | GI 307133762 NG_027719 Homo sapiens | GGATGTATGTGGAAA |
| NO: 1018 | glutathione-disulfide reductase | AGCTCTTCTTACTCC |
| | (GSR), RefSeqGene on chromosome 8 | AGGTAAGGTCTCTTC |
| | | CCTTCTTGGTGAGGG |
| SEQ ID | GI 315013554 NG_027996 Homo sapiens | TAGGATTTGCATCAG |
| NO: 1019 | granzyme A (GZMA), RefSeqGene on | ATGTCTGCGGGTCTA |
| | chromosome 5 | CATAGAGTAAAAGCA |
| | | GGGGAAATGCTCTCA |
| SEQ ID | GI 297515475 NG_021404 Homo sapiens | GGAAAAGATTGGTTT |
| NO: 1020 | matrix metallopeptidase 13 (MMP13), | AGAGAAGGCCCTCCT |
| | RefSeqGene on chromosome 11 | ATCTGCAGAGTTAGC |
| | | TGATCCACTGGAGAG |
| SEQ ID | GI 190014585 NM_021144 Homo sapiens | CTTGATACAGACTCA |
| NO: 1021 | PC4 and SFRS1 interacting protein 1 | TTGGACTGAATTTCC |
| | (PSIP1), transcript variant 1, mRNA | CCCTTCCCCCCATTG |
| | | ATGGAAGAATGTTCC |
| SEQ ID | GI 189909140 EU779708 Homo sapiens | ATGGCAGTTTTTTGG |
| NO: 1022 | protein tyrosine phosphatase-2 | CAAGGCCTAGTAAAA |
| | (PTPN11) mRNA, complete cds | GTAACCCTGGAGACT |
| | | TCACACTTTCCGTTA |
| SEQ ID | GI 1209857163 NM_015317 Homo sapiens | AGCAGCAGCTCTTTC |
| NO: 1023 | pumilio RNA binding family member 2 | AGAGGACTAATGCAC |
| | (PUM2), transcript variant 1, mRNA | TAACAGTTCAACAGT |
| | | TAACTGCAGCTCAAC |
| SEQ ID | GI 1209857163 NM_015317 Homo sapiens | TATTTCGGCCCGTAT |
| NO: 1024 | pumilio RNA binding family member 2 | CGAACTTTTGTTCTC |
| | (PUM2), transcript variant 1, mRNA | AGTATCAGCCCATGG |
| | | TTTCAGGATCAAAGC |
| SEQ ID | GI 115432768 DQ995342 Homo sapiens | AAGACTGGTCGGTTT |
| NO: 1025 | ADAM metallopeptidase domain 33 | GCATATGAAAGGCAT |
| | (ADAM33) gene, complete cds | GAGTTGTTTCTGATC |
| | | TCCAGGAATCAAGCA |
| SEQ ID | GI 37675288 AY436640 Homo sapiens | GAGTTCAGGTGAAGA |
| NO: 1026 | breast cancer 2, early onset | TGTGAAGGTGAGAGA |
| | (BRCA2) gene, complete cds | AGAGGATGGGTAGAC |
| | | ATTTCCCTGGTGAAG |
| SEQ ID | GI 58802450 AY916056 Homo sapiens | CAGTGGTGGTCAGGA |
| NO: 1027 | flavin containing monooxygenase 2 | ATAGCAAAAACTAAA |
| | (FM02) gene, complete cds | CTCCTTCTGCAAGGA |
| | | CAGACCTAGGCAAAG |
| SEQ ID | GI 221139813 NG_009361 Homo sapiens | CACAAGAAGCAAAAC |
| NO: 1028 | BCL2, apoptosis regulator (BCL2), | GCTGGAGAGGGACAG |
| | RefSeqGene on chromosome 18 | ATGTCTACAGAAGAT |
| | | GTTTTGGGACAGTTT |
| SEQ ID | GI 197313719 NG_008421 Homo sapiens | CTGTCTAACTTGCTA |
| NO: 1029 | cytochrome P450 family 3 subfamily | TCTATGGCAGGACCT |
| | A member 4 (CYP3A4), RefSeqGene on | TTGAAGGGTTCACAG |
| | chromosome 7 | GAAGCAGCACAAATT |
| SEQ ID | GI 197313719 NG_008421 Homo sapiens | AACATAAAGACAGCT |
| NO: 1030 | cytochrome P450 family 3 subfamily | TGTCTGAGGCAACAT |
| | A member 4 (CYP3A4), RefSeqGene | ACAATCCACCAAAGT |
| | on chromosome 7 | CACCTGCTTGTCTGT |
| SEQ ID | GI 345525392 NG_029921 Homo sapiens | AGTGAGCCCCATTCT |
| NO: 1031 | major histocompatibility complex, | GTGACAATCAGTAGG |
| | class II, DR beta 1 (HLA-DRB1), | ACTCAAGACATGCTG |
| | RefSeqGene on chromoso [symbol] | TAATCCTACCAATGT |
| SEQ ID | GI 224809291 NG_011495 Homo sapiens | TTGTTCTTTCTCAGC |
| NO: 1032 | interferon induced with helicase C | CACCACAGCTATTCC |
| | domain 1 (IFIH1), RefSeqGene on | AGCAGCTCATTTCTT |
| | chromosome 2 | CACTTCACATCTGTT |
| SEQ ID | GI 974987494 NG_011716 Homo sapiens | TCACAGTAAAAATGC |
| NO: 1033 | transcription factor 4 (TCF4), | TCTTTCCCCAGGTAC |
| | RefSeqGene on chromosome 18 | AGGATATATGGCCAT |
| | | CTGGCCTGGATATCA |
| SEQ ID | GI 14594701 AY037115 Homo sapiens | GTCTGGAACAAGTGT |
| NO: 1034 | thymic stromal lymphopoietin (TSLP) | CACAATTACAAGGAT |
| | mRNA, complete cds | TGTGGCGTCGCTTCA |
| | | ATCGACCTTTACTGA |
| SEQ ID | GI 58258007 AY902236 Homo sapiens | CAATGCTCATGTGAC |
| NO: 1035 | flavin containing monooxygenase 5 | AGCACACTCAAGTCA |
| | (FMO5) gene, complete cds | CAGTGGCTCATGGTA |
| | | TGAATAGTCACTGTG |
| SEQ ID | GI 166706816 EU332867 Homo sapiens | TTTCCTGGGGAGTTG |
| NO: 1036 | protein kinase C, epsilon (PRKCE) | ATTAAAGTTACAGAT |
| | gene, complete cds | TTTCAAGCCTCACCC |
| | | AGTGGAATTGGGGAG |
| SEQ ID | GI 212549766 NG_008985 Homo sapiens | GAAGGTGGGGCAATT |
| NO: 1037 | N-acylsphingosine amidohydrolase | CTGACTGAGTAAAGA |
| | 1 (ASAH1), RefSeqGene on | AGGAACAGCCATTCT |
| | chromosome 8 | GTTTAGCACGAGAAG |
| SEQ ID | GI 223671920 NG_009776 Homo sapiens | CACCTCTGGCGTTAA |
| NO: 1038 | epoxide hydrolase 1 (EPHX1), | GTCCTGACTCCTACC |
| | RefSeqGene on chromosome 1 | TGTTTCACCATCACT |
| | | GCAGTTTTAAGAGTA |
| SEQ ID | GI 570359607 NG_033871 Homo sapiens | GGGCTCAGGGAAGAA |
| NO: 1039 | F-box protein 38 (FBX038), | GAGAAGTAAGGATTA |
| | RefSeqGene on chromosome 5 | TAAGTCTACAGTGGA |
| | | GGCTTCAGTTGTGGC |
| SEQ ID | GI 341865572 NG_029566 Homo sapiens | GAAGGAGCCTTCTGA |
| NO: 1040 | nucleolar protein 3 (NOL3), | AGCAAATATCAGAGT |
| | RefSeqGene on chromosome 16 | CACTCCTCTGCTTAA |
| | | AGCTCCTCTCACTCC |
| SEQ ID | GI 1047600468 NM_001718 Homo sapiens | GCAATGCTCACCTCT |
| NO: 1041 | bone morphogenetic protein 6 | TCTTTACCAGAACGG |
| | (BMP6), mRNA | TTCTTTGACCAGCAC |
| | | ATTAACTTCTGGACT |
| SEQ ID | GI 428673540 NM_006273 Homo sapiens | CAGTGCTGTAAAAAC |
| NO: 1042 | C-C motif chemokine ligand 7 | TGTGGGATGCTCCTC |
| | (CCL7), mRNA | CCTTCTCTACCTCAT |
| | | GGGGGTATTGTATAA |
| SEQ ID | GI 14518294 AF390905 Homo sapiens | TGTCCCACCGTTAAC |
| NO: 1043 | interleukin 19 (IL19) gene, | CACTCCCTGCTTAGA |
| | complete cds | CTGAACAGTTAATCT |
| | | TCTGCAGAGTAACTG |
| SEQ ID | GI 14278707 AF365976 Homo sapiens | ACATCCAATCCATAT |
| NO: 1044 | interleukin 3 (IL3) gene, | CAAGGACGGTGACTG |
| | complete cds | GAATGAATTCCGGAG |
| | | GAAACTGACGTTCTA |
| SEQ ID | GI 94717611 DQ520733 Homo sapiens | CCTTGAGTTGTTGCA |
| NO: 1045 | UDP glucuronosyltransferase 2 | AGAAAGAATACTCCA |
| | family, polypeptide B4 (UGT2B4) | AGTGTGGGTAGAGGG |
| | gene, complete cds | GACGCAATGTGTAAG |
| SEQ ID | GI 983173900 NG_009770 Homo sapiens | CTGGAAACCAGTGAA |
| NO: 1046 | hexosaminidase subunit beta (HEXB), | AATTTCTGAGCAGAG |
| | RefSeqGene on chromosome 5 | GAGTAACATGATGGG |
| | | AACCACACTTTGGGA |
| SEQ ID | GI 342307070 NG_029606 Homo sapiens | GTTAGCCTTTGGTAG |
| NO: 1047 | hypoxia inducible factor 1 alpha | ATTCATCTGGTTAGG |
| | subunit (HIF1A), RefSeqGene on | CTCTGATAGAGAGTT |
| | chromosome 14 | TCCCTTGAGGCCAGG |
| SEQ ID | GI 335334929 NG_012849 Homo sapiens | TGGAAAAGAATTGGG |
| NO: 1048 | phosphoinositide-3-kinase regulatory | GTAGGGGTGTGGCAC |
| | subunit 1 (PIK3R1), RefSeqGene on | ATGGCACTTAAGTTG |
| | chromosome 5 | AGGTTTATGTTAGAG |
| SEQ ID | GI 341823644 NG_029509 Homo sapiens | AATCAAGGAACCCAC |
| NO: 1049 | prostaglandin E receptor 3 (PTGER3), | ACAGAAGGTGATCTC |
| | RefSeqGene on chromosome 1 | CATTGCTAACTGACC |
| | | AGCAAATTACACAGG |
| SEQ ID | GI 1024336768 NG_046906 Homo sapiens | CTGGATCATTTATTG |
| NO: 1050 | TANK binding kinase 1 (TBK1), | CCTGTATTGCTGACC |
| | RefSeqGene on chromosome 12 | TTTAGTCCCCAGCTC |
| | | CTTCCAGAAGTTTGG |
| SEQ ID | GI 169646347 NM_001115152 Homo sapiens | CGTGTTCACCGTGAC |
| NO: 1051 | CD300 molecule like family member d | CATGGAGAATCTCAA |
| | (CD300LD), mRNA | AAGAGATGATGCTGA |
| | | CAGTTATTGGTGTGG |
| SEQ ID | GI 974005408 NM_015133 Homo sapiens | GATTCTCCTCAGGCT |
| NO: 1052 | mitogen-activated protein kinase 8 | TTGGCCCTGCAAGCA |
| | interacting protein 3 (MAPK8IP3), | AACCCACATATCTGC |
| | transcript variant 1, [symbol] | TCTGTATGTAATAAA |
| SEQ ID | GI 740086238 NM_017921 Homo sapiens | CCTTATCCACCACGC |
| NO: 1053 | NPL4 homolog, ubiquitin recognition | TATTATAGTTTCCGT |
| | factor (NPLOC4), transcript variant | TGTCCTCCACCAGCA |
| | 1, mRNA | TTTCCCTTACTCTGA |
| SEQ ID | GI 38505193 NM_000955 Homo sapiens | GTGCGCGGCGCAGAG |
| NO: 1054 | prostaglandin E receptor 1 (PTGER1), | CCTTTGGGAATAAAA |
| | mRNA | AGCCATTCTGCGAAA |
| | | AAAAAAAAAAAAAAA |
| SEQ ID | GI 394582046 NM_003877 Homo sapiens | AAAGGTCCAGGCTCC |
| NO: 1055 | suppressor of cytokine signaling 2 | AGTAGGAGAGAAAGA |
| | (SOCS2), transcript variant 1, mRNA | ACTCCTCATAGGAAT |
| | | ACTGAAGAAGTGGGA |
| SEQ ID | ref \| NG_034014.1\| Homo sapiens | TCAGGGAAAGTGCCT |
| NO: 1056 | cyclin dependent kinase 2 (CDK2), | ATTGTAAAAAGAACT |
| | RefSeqGene on | GAGGGTGCTATGGTG |
| | | TTGAGCTGAGAGAGA |
| SEQ ID | GI 340523111 NG_029470 Homo sapiens | AGACCCATTAAGTAT |
| NO: 1057 | enolase 1 (ENO1), RefSeqGene on | ATTAATGAGTTCCTG |
| | chromosome 1 | GCACCAGCCCTGTGC |
| | | ACTCAGGTAACTGAT |
| SEQ ID | GI 383087750 NG_015839 Homo sapiens | CGAATTATAATTCCT |
| NO: 1058 | ERCC excision repair 1, endonuclease | CAAGCCCTCTTTCCC |
| | non-catalytic subunit (ERCC1), | CTCCCATCCTTTGCC |
| | RefSeqGene on chromo [symbol] | CATGCTGTTCTTTAT |
| SEQ ID | GI 237858716 NG_012099 Homo sapiens | AGGGACAAGGGAAAA |
| NO: 1059 | macrophage migration inhibitory factor | ATCCACTTCCAAGTT |
| | (MIF), RefSeqGene on chromosome 22 | CAATCTGGTTGTGAG |
| | | CAGAATTCAGTTCCT |
| SEQ ID | GI 343403834 NG_029695 Homo sapiens | ACAGGGTAAGTTTTT |
| NO: 1060 | prefoldin subunit 5 (PFDN5), RefSeqGene | CCTGGGGCACCTCTT |
| | on chromosome 12 | GACCCTATCTCCATA |
| | | ATAAGGAACATGGAT |
| SEQ ID | GI 1018443413 NG_027814 Homo sapiens | ACAATACTTGAACCA |
| NO: 1061 | SATB homeobox 1 (SATB1), RefSeqGene | CCCTCCCCCTGTCAG |
| | on chromosome 3 | TAGATCTATGAATAA |
| | | GCCTTTGGAGCAACA |
| SEQ ID | GI 238814313 NG_012262 Homo sapiens | TTTTGGTACCCATTT |
| NO: 1062 | topoisomerase (DNA) 1 (TOP1), | GATACACTGTGGCTG |
| | RefSeqGene on chromosome 20 | GTAGGTAAGTAGGTT |
| | | GGTGTAAGTGGACCT |
| SEQ ID | GI 211971007 NM_001136538 Homo sapiens | GGCCCATTGGAACTA |
| NO: 1063 | acyl-CoA dehydrogenase family member 10 | CTTCAGTTTGATCAC |
| | (ACAD10), transcript variant 1, mRNA | GGGCAGTCAAATCCA |
| | | ACTTACTACATCAGG |
| SEQ ID | GI 38708325 NM_001896 Homo sapiens | TGCGACTGATAGATT |
| NO: 1064 | casein kinase 2 alpha 2 (CSNK2A2), | GGGGTCTGGCAGAAT |
| | mRNA | TCTATCATCCTGCTC |
| | | AGGAGTACAATGTTC |
| SEQ ID | GI 576795860 NM_001549 Homo sapiens | CGCTACTGCAACCTT |
| NO: 1065 | interferon induced protein with | CAGAAATATAATGGG |
| | tetratricopeptide repeats 3 | AAGTCTGAAGACACT |
| | (1 FITS), transcript variant 1, mR [symbol] | GCTGTGCAACATGGT |
| SEQ ID | GI 633257761 NM_001143836 Homo sapiens | TCATGGTGGATTCAA |
| NO: 1066 | NADPH oxidase 4 (NOX4), transcript | GAACTCCCTAGTGAG |
| | variant NOX4B, mRNA | GAGCTGAACTTGCTC |
| | | AATCTAAGGCTGATT |
| SEQ ID | GI 23510408 NM_003057 Homo sapiens | AAAGAATGGGAAGTT |
| NO: 1067 | solute carrier family 22 member 1 | GCCTCCTGCTGATTT |
| | (SLC22A1), transcript variant 1, mRNA | AAAGATGCTTTCCCT |
| | | CGAAGAGGATGTCAC |
| SEQ ID | GI 315360639 NM_012474 Homo sapiens | CTTCCTGATAGAAGA |
| NO: 1068 | uridine-cytidine kinase 2 (UCK2) | TGGGAAGGAGAGGGC |
| | , mRNA | AGAACTTTGCCTGCT |
| | | GTCATATCTGAAGAA |
| SEQ ID | GI 94717638 DQ496106 Homo sapiens | AAGTGAAAACACACA |
| NO: 1069 | aconitase 1, soluble (ACO1) gene, | GTATCTCTAAGGCCT |
| | complete cds | AGCTAAGGACCAGCA |
| | | CAGCATCACTTCTGT |
| SEQ ID | GI 87046146 DQ142911 Homo sapiens | TGCCTGATCTTTGCA |
| NO: 1070 | cogulation factor VII (F7) gene, | AAATCAGGAGCATTG |
| | complete cds | GATCAAAGCTATGTG |
| | | AAGATCCTGTGAGGT |
| SEQ ID | GI 77702091 DQ217939 Homo sapiens | CTCTTTGGATCACCA |
| NO: 1071 | mannose-binding protein C (MBL2) | GCTTTCAGCTCAGGG |
| | gene, complete cds | CCTGCCAATGAGTAA |
| | | ATGATAGTTAACAGG |
| SEQ ID | GI 14278706 AF378903 Homo sapiens | ACATAAAATCGCTCA |
| NO: 1072 | protein C (PROC) gene, complete cds | CTCTGTGCCTCACAT |
| | | CAAAGGGAGAAAATC |
| | | TGATTGTTCAGGGGG |
| SEQ ID | GI 1206876284 NG_033870 Homo sapiens | TACACTGAGGCTGCT |
| NO: 1073 | asparagine synthetase (glutamine- | TTAAGTTGCACATTG |
| | hydrolyzing) (ASNS), RefSeqGene | ATCTGAAGCCTTGGG |
| | on chromosome 7 | CTTCTGTTCCTATTC |
| SEQ ID | GI 654825103 NG_029876 Homo sapiens | GCTCTCTTGGTATCC |
| NO: 1074 | C-C motif chemokine ligand 23 (CCL23), | GTCTTTGTCTGGCAT |
| | RefSeqGene on chromosome 17 | CTAGGAAGAATCAGG |
| | | TCACATCAACCTGAA |
| SEQ ID | GI 190358507 NG_008042 Homo sapiens | TGAGTTAACGAGGCC |
| NO: 1075 | 2,4-dienoyl-CoA reductase 1 (DECR1), | TGTGGCAAGCTTTTG |
| | RefSeqGene on chromosome 8 | GTGTGGTACTCAGTT |
| | | ATTTTAATGTCTGCA |
| SEQ ID | GI 212549732 NG_008981 Homo sapiens | GTTGTAAATATTTTA |
| NO: 1076 | growth differentiation factor 6 | TGGGAAGGCAGCCCA |
| | (GDF6), RefSeqGene on chromosome 8 | GGAAAATCTGGTTAG |
| | | CTTTCCGCCTTGCAC |
| SEQ ID | GI 254911084 NG_012516 Homo sapiens | ACTCACATCTGTATG |
| NO: 1077 | moesin (MSN), RefSeqGene on | CAAATGCAGCATCCT |
| | chromosome X | CTCAACCATGCCTCT |
| | | TCTTTTCCTCAAGTT |
| SEQ ID | GI 224809269 NG_011475 Homo sapiens | TAATATTGACAGGAA |
| NO: 1078 | toll like receptor 4 (TLR4), | ACCCCATCCAGAGTT |
| | RefSeqGene on chromosome 9 | TAGCCCTGGGAGCCT |
| | | TTTCTGGACTATCAA |
| SEQ ID | GI 1010228574 NM_001321573 Homo sapiens | AGGGGTACCCAGATA |
| NO: 1079 | calcium/calmodulin dependent protein | GTCCTTGTTTTAATG |
| | kinase II delta (CAMK2D), transcript | ATGGGTTCTCTCACC |
| | variant 14, | ACTGTCTTGATGCTC |
| SEQ ID | GI 50234968 AY665769 Homo sapiens | GAAAGTGCTTTTCTG |
| NO: 1080 | chemokine-like factor (CKLF) gene, | CTAGAAAAGCAGCAG |
| | complete cds | CATATGTGAGGGTCT |
| | | AGGTGGAGAAAGAGC |
| SEQ ID | GI 440503006 JX512449 Homo sapiens | GGTGCCTCAGTTTTC |
| NO: 1081 | mitogen-activated protein kinase 1 | TCTGTAGCACCCTCA |
| | (MAPK1) gene, complete cds | AGAAGATATGCTGAG |
| | | AGTAATGAGGGTCAG |
| SEQ ID | GI 1049011036 AH002959 Homo sapiens | GCCTCCCCAGATCTT |
| NO: 1082 | RAF1 | AGTAAGCTATATAAG |
| | (RAF1) gene, complete cds | AACTGCCCCAAAGCA |
| | | ATGAAGAGGCTGGTA |
| SEQ ID | GI 344925847 NG_029819 Homo sapiens | CAATCCCAGCTGACA |
| NO: 1083 | prolactin | GCCAGTATCAACTAA |
| | (PRL), RefSeqGene on chromosome 6 | CAAACATGGTTTGTT |
| | | TAGCTCTAGCCACCA |
| SEQ ID | GI 149274622 NM_032579 Homo sapiens | TCTAGAGTACAGTCC |
| NO: 1084 | resistin like beta | CTCTCCTATAAGCAA |
| | (RETNLB), mRNA | GAAGCTCTCGTGTGC |
| | | TAGTGTCAAAAGCCA |
| SEQ ID | GI 94717638 DQ496106 Homo sapiens | CTGTAGCATGGGCTG |
| NO: 1085 | aconitase 1, soluble | CATGATGTTCTGATT |
| | (ACO1) gene, complete cds | TCTCCTACACAGTCT |
| | | CCATTCTCAGTCTGA |
| SEQ ID | GI 294489317 NG_017064 Homo sapiens | CCCTAGGAAGGGGGA |
| NO: 1086 | acyl-CoA dehydrogenase family member 9 | AACGCTACTGTGTTT |
| | (ACAD9), RefSeqGene on chromosome 3 | ACTCCTATTTTGTAG |
| | | ATGAGCACACTGAGG |
| SEQ ID | GI 190194409 NG_008004 Homo sapiens | GGGTTTTAGCCTTGG |
| NO: 1087 | activin A receptor type 1 | GCTGGAGAAGCCATA |
| | (ACVR1), RefSeqGene on chromosome 2 | GGGCTTGGTGAATAA |
| | | ATTACTTTTGCAATG |
| SEQ ID | GI 219842221 NG_009291 Homo sapiens | GAATGTGCTGATGAC |
| NO: 1088 | albumin | AGGGTAAAGAGTCGT |
| | (ALB), RefSeqGene on chromosome 4 | CGATATGCTTTTTGG |
| | | TAGCTTGCATGCTCA |
| SEQ ID | GI 1025608649 NG_047040 Homo sapiens | GAGAACAGTATAGCC |
| NO: 1089 | calcium/calmodulin dependent | CAGGATTGAAAGCCC |
| | protein kinase 11 alpha | AGCTCTGGAGTCTAA |
| | (CAMK2A), RefSeqGene on chromos [symbol] | CTGTCTGGGTAAAAA |
| SEQ ID | GI 324021728 NG_028155 Homo sapiens | GCAACTATTATAAGA |
| NO: 1090 | indoleamine 2,3-dioxygenase 1 | TGCTCTGAAAACTCT |
| | (IDO1), RefSeqGene on chromosome 8 | TCAGACACTGAGGGG |
| | | CACCAGAGGAGCAGA |
| SEQ ID | GI 514825583 NG_033271 Homo sapiens | GCTTTTCATGGTGGC |
| NO: 1091 | Kruppel like factor 10 | AGGTGTCCAGAAGTG |
| | (KLF10), RefSeqGene on chromosome 8 | GGAATGGAGGAAGAT |
| | | GACTGAAATATTCTC |
| SEQ ID | GI 686223163 NG_034261 Homo sapiens | GCCTGTTCCAGTTTT |
| NO: 1092 | primase | AGTACTGGAAGTCCC |
| | (DNA) subunit 2 | ACATCCTTAGAAACC |
| | (PRIM2), RefSeqGene on chromosome 6 | TCTCATTCCTGGACA |
| SEQ ID | GI 343488503 NG_029728 Homo sapiens | CCTTCACACAATCTC |
| NO: 1093 | protein kinase cAMP-activated | CAAGTCACCAACCAT |
| | catalytic subunit beta | GCTAGTTTCAGCATT |
| | (PRKACB), RefSeqGene on chromosom [symbol] | CCTGTTGGTAAAGGA |
| SEQ ID | GI 257153311 NG_012800 Homo sapiens | ATTCCATGCACAAAA |
| NO: 1094 | regulator of G protein signaling 2 | AGAGTGTAGCAGCAG |
| | (RGS2), RefSeqGene on chromosome 1 | GAACACACAACAATT |
| | | CCCTTACTCAGACCT |
| SEQ ID | GI 315365058 HQ384160 Homo sapiens | GAAAACCTTTCTCCC |
| NO: 1095 | fatty acid binding protein 4, adipocyte | TCAACCCCATGCATG |
| | (FABP4) gene, complete cds | ACAGTCCAATAAAGT |
| | | CAGAACTGGGGTTAG |
| SEQ ID | GI 57864635 AY882422 Homo sapiens | ACATCTTGAGTTCCC |
| NO: 1096 | flavin containing monooxygenase 4 | ATACAGGCTCTGCTA |
| | (FM04) gene, complete cds | CTTACTGTATGGTTC |
| | | TGGGCAAGTTGCTTA |
| SEQ ID | GI 14423570 AF387519 Homo sapiens | GAGGGAGAGCAATGT |
| NO: 1097 | interleukin 22 | TGTTCAGAGAAAGAT |
| | (IL22) gene, complete cds | CAACAGGAGGAGAAA |
| | | CTGTCAGAGCTGTCT |
| SEQ ID | GI 27805154 AY217549 Homo sapiens | GGACACCAGTGTATG |
| NO: 1098 | mutL homolog 1, colon cancer, | TTGGGATGCAAACAG |
| | nonpolyposis type 2 | GGAGGCTTATGACAT |
| | (E. coli) | CTAATGTGTTTTCCA |
| | (MLH1) gene, complete cds | |
| SEQ ID | GI 297139781 NG_021374 Homo sapiens | TAGGATTGGTATGTT |
| NO: 1099 | early growth response 1 | TTTCCCCATCTGCAC |
| | (EGR1), RefSeqGene on chromosome 5 | AGCTTTCCAGAAAAG |
| | | GGTCACTGGAGTTTT |
| SEQ ID | GI 908212454 NG_042284 Homo sapiens | CAGAGAAGAGGCTGA |
| NO: 1100 | SMAD family member 1 | ATTAAAAGAAGCTGA |
| | (SMAD1), RefSeqGene on chromosome 4 | AAAGGGGCAAAAGAG |
| | | GCATCCAGAGAAGGT |
| SEQ ID | GI 545687789 NM_015901 Homo sapiens | GAAGCATGGTTTGCT |
| NO: 1101 | nudix hydrolase 13 | CTGGATCTAGGTCTG |
| | (NUDT13), transcript variant 1, mRNA | GATAGCTCCTTTTCC |
| | | ATAAGTGCCTCCTTA |
| SEQ ID | GI 257743127 NG_012865 Homo sapiens | TTCCACGTTCAATGT |
| NO: 1102 | amylo-alpha-1, 6-glucosidase, | TGTCGTGTTGGTAGC |
| | 4-alpha-glucanotransferase | TTAAGATTGCCCATG |
| | (AGL), RefSeqGene on chromoso [symbol] | TCGGGATTATTTCCA |
| SEQ ID | GI 343168757 NG_029673 Homo sapiens | CTTCTGGTTGAGCAC |
| NO: 1103 | Fos proto-oncogene, AP-1 | TTGGTTTGCCTGGAT |
| | transcription factor subunit | GATGCTCTGGCCTTA |
| | (FOS), RefSeqGene on chromosome 1, [symbol] | CTCAATCTTTATCAT |
| SEQ ID | GI 168480137 NG_007294 Homo sapiens | GCACATGAGCAGGAC |
| NO: 1104 | hydroxyacyl-CoA dehydrogenase/ | TGAAAGTATTTGTTC |
| | 3-ketoacyl-CoA | TGTTCCACCTCTGTA |
| | thiolase/enoyl-CoA hydratase | CCTGGCAGAAATGTT |
| | (trifunctiona [symbol] | |
| SEQ ID | GI 432134283 NG_033039 Homo sapiens | AGTTCCCCTTCTCTA |
| NO: 1105 | lymphotoxin beta receptor | TAACCCAAGGGAAGT |
| | (LTBR), RefSeqGene on chromosome 12 | TTCTCTCATTCTGCC |
| | | TTTAGGAGCTGCATT |
| SEQ ID | GI 1154067879 NG_052976 Homo sapiens | GAGCACTATGCTGGC |
| NO: 1106 | malate dehydrogenase 2 | TGTATTTGGGGTACA |
| | (MDH2), RefSeqGene on chromosome 7 | TTTGCCATTTTATGG |
| | | GATCCTTGCAACAAC |
| SEQ ID | GI 215490061 NG_009062 Homo sapiens | TCAAAAAAGCGGCAT |
| NO: 1107 | nuclear receptor subfamily | GGAAATTCATTGGGC |
| | 3 group C member 1 | TCCATTCGATACCTC |
| | (NR3C1), RefSeqGene on chromosome 5 | GTGTTTAGAGATCGT |
| SEQ ID | GI 51479142 NM_001003703 Homo sapiens | ACCTGTTGATGCTAG |
| NO: 1108 | ATP synthase, H+ transporting, | TTCAGAGTATCAGCA |
| | mitochondrial Fo complex subunit F6 | AGAGCTGGAGAGGGA |
| | (ATP5J), transcript v [symbol] | GCTTTTTAAGCTCAA |
| SEQ ID | GI 84043946 NM_030661 Homo sapiens | GTGGCCAAACAAATC |
| NO: 1109 | homeobox A3 | TTCCCCTGGATGAAA |
| | (HOXA3), transcript variant 1, mRNA | GAGTCTCGACAAAAC |
| | | ACAAAGCAGAAAACC |
| SEQ ID | GI 62122910 NM_001014336 Homo sapiens | ATTTCTCAACAGTTT |
| NO: 1110 | interleukin 31 | TCAGAGTGCATGGAC |
| | (IL31), mRNA | CTCGCACTAAAATCA |
| | | TTGACCTCTGGAGCC |
| SEQ ID | GI 208431777 NM_001135638 Homo sapiens | AACCAGAGCGTGATG |
| NO: 1111 | phosphatidylinositol-4-phosphate | TCCTCATGCAAGATT |
| | 5-kinase type 1 alpha | TCTACGTGGTTGAGA |
| | (PIP5K1A), transcript variant 1, [symbol] | GTATCTTCTTTCCCA |
| SEQ ID | GI 149192859 NM_012192 Homo sapiens | TAAAAGTTGCTGACT |
| NO: 1112 | translocase of inner mitochondrial | AGGACCTTACCCACA |
| | membrane 10B | GGGTGGCACTCTGTC |
| | (TIMM10B), mRNA | CTTGGGAAATATAAA |
| SEQ ID | ref\|NG_009824.2\| Homo sapiens | GCCTCAGTTGGAATC |
| NO: 1113 | crystallin alpha B | TTGGTTTACTTGCTT |
| | (CRYAB), RefSeqGene on chromosome | ACCAGCCATGTGACC |
| | | TTAGGCAAGTTAGTT |
| SEQ ID | GI 208022628 NM_004887 Homo sapiens | CCTCCCCTCACCACA |
| NO: 1114 | C-X-C motif chemokine ligand 14 | TTGAGAAATCTCAGT |
| | (CXCL14), mRNA | GAGTCACCGAGTGGT |
| | | TCTGCATATTAATGA |
| SEQ ID | GI 545687789 NM_015901 Homo sapiens | GGAAGCATTAGTTTG |
| NO: 1115 | nudix hydrolase 13 | GATGTAGGCCCTTGT |
| | (NUDT13), transcript variant 1, mRNA | TCAGTCATTTTCTCT |
| | | AAGGCCTTGGAAGCA |
| SEQ ID | GI 1036032346 AH001409 Homo sapiens | AGCAGCCAGGATCAT |
| NO: 1116 | alcohol dehydrogenase 6 | TGGAGTGGATGTCAA |
| | (ADH6) gene, complete cds | CAAGGAGAAATTTAA |
| | | GAAGGCACAGGAATT |
| SEQ ID | GI 117606581 EF064765 Homo sapiens | GAAACAAAGATCTCT |
| NO: 1117 | chemokine | GATAAACATAACAAC |
| | (C-C motif) ligand 21 | ATAGCAGGCTGGGCG |
| | (CCL21) gene, complete cds | CAGTGGCTCATGTCT |
| SEQ ID | GI 21435968 AF518727 Homo sapiens | CTTCTGGATTGGTTA |
| NO: 1118 | cyclin El | ATGGAGGTGAGCTTG |
| | (CCNEl) gene, complete cds | AGTCTTCCTGTTCGC |
| | | TTCATGAAAGCACTG |
| SEQ ID | GI 4572463 AF123653 Homo sapiens | TCAAACGACAGAGGA |
| NO: 1119 | FEZI | GCTTTTCTGGAATTT |
| | (FEZl) gene, complete cds | CAAACCATTGCTCTT |
| | | AGTCCCAAGCTAGGC |
| SEQ ID | GI 291084732 NG_016743 Homo sapiens | ACCTATTTTCCTCGG |
| NO: 1120 | oxidized low density | GCTCATTTAACTGGG |
| | lipoprotein receptor 1 | AAAAGAGCCAAGAGA |
| | (OLR1), RefSeqGene on chromosome 12 | AGTGCTTGTCTTTGG |
| SEQ ID | GI 1026191100 NG_047066 Homo sapiens | TTCTGTCACTGTATA |
| NO: 1121 | proliferating cell nuclear antigen | AGTGATATAAGTGCA |
| | (PCNA), RefSeqGene on chromosome 20 | CCTCTAGCTGGACAG |
| | | CTCCTCAGGTCCTCC |
| SEQ ID | GI 225579099 NG_011677 Homo sapiens | TAAGAAGGGGAGACA |
| NO: 1122 | pyruvate kinase, liver and RBC | GAAGCCATGGAACAT |
| | (PKLR), RefSeqGene on chromosome 1 | AGGAGGAAAATGAGG |
| | | GTGAAAACTAGGAGC |
| SEQ ID | GI 209447082 NG_008731 Homo sapiens | ATTAGAGGTGCTGGA |
| NO: 1123 | vitamin D receptor | CCAAACTGTGCTCTA |
| | (VDR), RefSeqGene on chromosome 12 | CTCCCAGGAAGTGTG |
| | | CAGTCAATATATGAC |
| SEQ ID | GI 38327624 NM_004077 Homo sapiens | CATAGAACTCCGGAA |
| NO: 1124 | citrate synthase | TGACCAGCATGATAT |
| | (CS), mRNA | TTTCAGAGTCTTGTC |
| | | CCCGGGGTATTAGCA |
| SEQ ID | GI 387598072 NM_005277 Homo sapiens | ACATAAATGTGCCAA |
| NO: 1125 | glycoprotein M6A | CAGCTTGATGGTGGC |
| | (GPM6A), transcript variant 1, mRNA | GTTTTTGAAATGTAG |
| | | AACAGAGTGCTTGCA |
| SEQ ID | GI 1168024004 KY497357 Homo sapiens | CATCAGTTCAGAAGC |
| NO: 1126 | HLA-DRA | ATTTCCAGATCCTGG |
| | (HLA-DRA) gene, complete cds | CTCCTTTCAGGTTAG |
| | | GGTCAATTCATTGCA |
| SEQ ID | GI 457232 L26524 Homo sapiens | CGATGCATTTACAGA |
| NO: 1127 | PRKDC gene, complete sequence | GAACATGGCAGGAGA |
| | | GAATCAGCTGCTGGA |
| | | GAGGAGAAGACTTTA |
| SEQ ID | GI 14488407 AF386492 Homo sapiens | TTTCCCAAGCAGGAG |
| NO: 1128 | serine-cysteine proteinase | ACATCAGGATAATGG |
| | inhibitor clade E member 1 | GAACAGAAGACAGGA |
| | (SERPINEl) gene, complete cds | GGTTTATCCCATGAA |
| SEQ ID | GI 205361192 NG_008140 Homo sapiens | TTTCCCGCAGCAACT |
| NO: 1129 | aminoadipate-semialdehyde synthase | TATACCATTTGACTG |
| | (AASS), RefSeqGene on chromosome 7 | CTCCTTGAGCTGTGA |
| | | TAAGAGATGCCTTTG |
| SEQ ID | GI 315013553 NG_027995 Homo sapiens | TCCCCATGCCTCTTT |
| NO: 1130 | cystatin A | GCTGTTTGTGGAAAA |
| | (CSTA), RefSeqGene on chromosome 3 | TAAAGCATTCTATAG |
| | | GCGGAGCTAGTGAAC |
| SEQ ID | GI 678246479 NG_034239 Homo sapiens | CTGAGGAACACAACA |
| NO: 1131 | lipopolysaccharide binding protein | AAATGGTCTACTTTG |
| | (LBP), RefSeqGene on chromosome 20 | CCATCTCGGATTATG |
| | | TCTTCAACACGGCCA |
| SEQ ID | GI 224809324 NG_011504 Homo sapiens | TGGCTTATAAAGAAG |
| NO: 1132 | NAD(P)H quinone dehydrogenase 1 | GCCATCTGAGCCCAG |
| | (NQO1), RefSeqGene on chromosome 16 | ATATTGTGGCTGAAC |
| | | AAAAGAAGCTGGAAG |
| SEQ ID | GI 296010927 NM_172213 Homo sapiens | CTACAACCTCACAGA |
| NO: 1133 | CD8b molecule | AGCTGCTTAACCCAT |
| | (CD8B), transcript variant 2, mRNA | GGATCCTGAAAACAT |
| | | AGGCAAGAAGCACAG |
| SEQ ID | GI 299758475 NM_001190737 Homo sapiens | AACAGCACAGCGGGA |
| NO: 1134 | nuclear factor 1 B | TCCTAGCAGTGCAAG |
| | (NFIB), transcript variant 1, mRNA | TTCAAAAGTAAGTCC |
| | | AATCATTTCTGTGAT |
| SEQ ID | GI 1006833939 NM_003978 Homo sapiens | CACTCCTTCCTCATT |
| NO: 1135 | proline-serine-threonine | TTGCTGCTGATTCTA |
| | phosphatase interacting protein 1 | GCCCCAAACAAAACA |
| | (PSTPIP1), transcript variant 1, | GGTTGAGCTTTTTCC |
| SEQ ID | GI 48762927 NM_002881 Homo sapiens | GCCGAATTCAGGGAA |
| NO: 1136 | RAS like proto-oncogene B | CAGATTCTCCGTGTG |
| | (RALB), mRNA | AAGGCTGAAGAAGAT |
| | | AAAATTCCACTGCTC |
| SEQ ID | GI 190194387 NG_008002 Homo sapiens | CATTGAAGCTGGAAG |
| NO: 1137 | acyl-CoA dehydrogenase, long chain | CCATCATACCTTACT |
| | (ACADL), RefSeqGene on chromosome 2 | GCCTTGAAACCCCTA |
| | | GGACTCAGCTAAGTA |
| SEQ ID | GI 294610641 NG_017089 Homo sapiens | CTTTGGGCCACCATG |
| NO: 1138 | galectin 3 | TTATCTCTCTGAGCC |
| | (LGALS3), RefSeqGene on chromosome 14 | TCAGTTTCCTTATGG |
| SEQ ID | GI 257153325 NG_012807 Homo sapiens | AGGTTGTGGTTTCAC |
| NO: 1139 | ST3 beta-galactoside alpha-2,3- | TTGTGTAATCCTGAG |
| | sialyltransferase 5 (ST3GAL5), | AGCTTGACAGCTTCG |
| | RefSeqGene on chromosome [symbol] | AGGAGTGTCTAACAT |
| SEQ ID | GI 728797213 LN680437 Homo sapiens | CTTGTTGCTGGACTA |
| NO: 1140 | mRNA for CD47 | GTGATCACTGTCATT |
| | | GTCATTGTTGGAGCC |
| | | ATTCTTTTCGTCCCA |
| SEQ ID | GI 226958454 NG_011902 Homo sapiens | TCTTAAGTAGTCACC |
| NO: 1141 | CCHC-type zinc finger nucleic acid | TTTGTAACCCTTGCA |
| | binding protein (CNBP), RefSeqGene | ATGCTGAGCATGAGA |
| | on chromosome 3 | CTGAACACTGGAGGA |
| SEQ ID | GI 224809262 NG_011468 Homo sapiens | ATCTGAGAAATGATG |
| NO: 1142 | matrix metallopeptidase 9 (MMP9), | AGAGATGGGATGAAC |
| | RefSeqGene on chromosome 20 | TGCAGACCATCCATG |
| | | GGTCAAAGAACAGGA |
| SEQ ID | GI 170014708 NG_007458 Homo sapiens | GGCTTGAATTGGTTG |
| NO: 1143 | notch 1 (NOTCH1), RefSeqGene on chromosome 9 | TTTAAAGCTGGGTAA |
| | | ATGCCCCCTTGACAT |
| | | TCTGTTGACACTGTC |
| SEQ ID | GI 226437630 NG_011780 Homo sapiens | CTGTGTCTTTGCTCA |
| NO: 1144 | sphingomyelin phosphodiesterase 1 | TGTTGGCCCTCTGGA |
| | (SMPD1), RefSeqGene on chromosome 11 | AATGATTTCCCCCTT |
| | | TTTTTTAAGTGCTCC |
| SEQ ID | GI 166795249 NM_006845 Homo sapiens | CAATCTTTGAAGGTG |
| NO: 1145 | kinesin family member 2C (KIF2C), | GAAAAGCAACTTGTT |
| | transcript variant 1, mRNA | TTGCATATGGCCAGA |
| | | CAGGAAGTGGCAAGA |
| SEQ ID | GI 193083137 NM_005512 Homo sapiens | GGGAAACTGAGGCTT |
| NO: 1146 | leucine rich repeat containing 32 | AGGAAGAGAATGAAA |
| | (LRRC32), transcript variant 1, mRNA | CTTGTGGAGAGCTTA |
| | | TACAGAGCCTCTGGG |
| SEQ ID | GI 336020352 NM_004834 Homo sapiens | AAGAATATAAAAGGC |
| NO: 1147 | mitogen-activated protein kinase | AACTGCTGGCAGAGA |
| | kinase | GACAGAAGCGGATTG |
| | kinase kinase 4 (MAP4K4), transcript | AGCAGCAGAAAGAAC |
| | variant 1, Mr [symbol] | |
| SEQ ID | GI 2735714 AF004877 Homo sapiens | TGCGTGACTTTGGGC |
| NO: 1148 | pro-alpha 2(1) collagen (COL1A2) | AAGTCAAACGGCCTT |
| | gene, complete cds | TTCTAAAATGTGAGA |
| | | CAGAGATTAAAGGGA |
| SEQ ID | GI 237820664 NG_012051 Homo sapiens | ACTGTCTGTAAAATA |
| NO: 1149 | caveolin 1 (CAV1), RefSeqGene on | ATATTCAGTCTGGCC |
| | chromosome 7 | TCAACCTGTCTTTCC |
| | | AGCCTCGGTGACACA |
| SEQ ID | GI 648216382 NG_029573 Homo sapiens | TTGTTTCTCAAGACC |
| NO: 1150 | TNF receptor superfamily member 8 | AGTTTCCCTGATGGC |
| | (TNFRSF8), RefSeqGene on chromosome 1 | CTTGGCAGTTTTTGA |
| | | GTATTGATCAGGTGT |
| SEQ ID | GI 187761339 NM_130767 Homo sapiens | CTTCCTTACTTTGCT |
| NO: 1151 | acyl-CoA thioesterase 12 (ACOT12), mRNA | GGAAATCTTGGTGGC |
| | | TGGTCAAAATCCATT |
| | | GAAGAAACAGCAGCC |
| SEQ ID | GI 54144649 NM_002090 Homo sapiens | CCCTTACCAGAGCTG |
| NO: 1152 | C-X-C motif chemokine ligand 3 | AAAATGAAAAAGAGA |
| | (CXCL3), mRNA | ACAGCAGCTTTCTAG |
| | | GGACAGCTGGAAAGG |
| SEQ ID | GI 52854078 AY755659 Homo sapiens | ATGAATCCCATTTGC |
| NO: 1153 | small proline-rich protein 1A (SPRRIA) | CTATTGACCCTGCAG |
| | gene, complete cds | TTAGCATGCTGTCAC |
| | | CCTGAATCATAATCG |
| SEQ ID | GI 345478675 NG_029883 Homo sapiens | GGGAAACTCTGCTGT |
| NO: 1154 | death associated protein kinase 1 (DAPK1), | CAACTAAGCAAACTC |
| | RefSeqGene on chromosome 9 | TAAATTTAACATTTG |
| | | GGGGACCCCCTTGGA |
| SEQ ID | GI 982742415 NM_020530 Homo sapiens | CAGGTCACTTTACCT |
| NO: 1155 | oncostatin M (OSM), transcript variant 1, | CTGTGGACCTGTTTT |
| | mRNA | CTCTTTGTGAAGCTA |
| | | GGGAGTTAGAGGCTG |
| SEQ ID | GI 152112977 EU012027 Homo sapiens | GTATTTTAAACCCGG |
| NO: 1156 | high-mobility group box 1 (HMGB1) gene, | CTGGCTGGATCATGT |
| | complete cds | CACTTTCTTATAGAA |
| | | GCCCTGCCTTTCCAG |
| SEQ ID | ref \| NG_033066.2\| Homo sapiens | AAGTGAAGGGAAGGG |
| NO: 1157 | C-C motif chemokine ligand 4 (CCL4), | GAGTTGGGATGGCCT |
| | RefSeqGene | GTAAGATTAGAGTGT |
| | | GGATTTTGAGAAAGA |
| SEQ ID | GI 237858716 NG_012099 Homo sapiens | ATTTAGGCCACTTGG |
| NO: 1158 | macrophage migration inhibitory factor | TGCTCATAACACCTC |
| | (MIF), RefSeqGene on chromosome 22 | TATGTCAGGTGAACA |
| | | CTATTGTCATCCCCA |
| SEQ ID | GI 170014713 NG_007461 Homo sapiens | CAGTTCTTCCTTGGT |
| NO: 1159 | transforming growth factor beta receptor | CATGGCTTACGTAAC |
| | 1 (TGFBR1), RefSeqGene on chromosome 9 | ACAGAGTTCTCTGAA |
| | | CAGATGGACTGTGGT |
| SEQ ID | GI 359338962 NG_031836 Homo sapiens | CGTCCATCGAGACTC |
| NO: 1160 | Z-DNA binding protein 1 (ZBP1), | ATTTGGTCGTAGATG |
| | RefSeqGene on chromosome 20 | ATAGAAAGCTGAGAC |
| | | CTGCTTCAGCACAAA |
| SEQ ID | GI 182995 M68891 Human GATA-binding | TTGTTTGGAGAACAA |
| NO: 1161 | protein (GATA2) mRNA, complete cds | CAAGGACAACTTTAT |
| | | GTAGAGAAAAGGAGG |
| | | GGACGGGACAGACGA |
| SEQ ID | GI 313665837 NM_152230 Homo sapiens | TTCTCAGCCTTCTCT |
| NO: 1162 | inositol polyphosphate multikinase | GAGGGCACTTTGCAA |
| | (IPMK), mRNA | ATACCTGAGTCCAAA |
| | | CAGAAGTACCAACTA |
| SEQ ID | GI 190014585 NM_021144 Homo sapiens | ACTTTCAGACTGCTC |
| NO: 1163 | PC4 and SFRS1 interacting protein 1 | ACAGAAGGAATATGC |
| | (PSIP1), transcript variant 1, mRNA | TGAAAGGCCAACATG |
| | | AGAAAGAAGCAGCAG |
| SEQ ID | GI 209571510 NM_015000 Homo sapiens | TCTTGGCACTGGAAG |
| NO: 1164 | serine/threonine kinase 38 like | GGTAGTTCTGGAAAG |
| | (STK38L), mRNA | CTACTTTGTTGAGAG |
| | | TCTCATTCTTCCCTG |
| SEQ ID | GI 82583661 DQ297144 Homo sapiens | AATGGAAGCAAAGAA |
| NO: 1165 | metallothionein 3 (growth inhibitory | ACAGATGACTGGCTG |
| | factor (neurotrophic)) (MT3) gene, | ATGCTAGAGTTGGGG |
| | complete cds | AACTCTGAGATCTGA |
| SEQ ID | GI 395393980 NG_011966 Homo sapiens | CCACCTCCCAGAGTT |
| NO: 1166 | mitogen-activated protein kinase kinase | CTTAACAGATGTGAA |
| | kinase 7 (MAP3K7), RefSeqGene on | AGTGCCAGGGCACAC |
| | chromosome 6 | AGTAGATACTAATCC |
| SEQ ID | GI 256985178 NG_012796 Homo sapiens | CTTGCCCCTGCTCAA |
| NO: 1167 | glycogen phosphorylase L (PYGL), | TTATCATTGGCTCCT |
| | RefSeqGene on chromosome 14 | CTTGCCTGCCATAGA |
| | | ACTTAAGCAAGTATT |
| SEQ ID | GI 523498473 NM_006332 Homo sapiens | TTTTTCTGATCCAGA |
| NO: 1168 | IFI30, lysosomal thiol reductase | CCCTCGGCACCTGCT |
| | (IFI30), mRNA | ACTTACCAACTGGAA |
| | | AATTTTATGCATCCC |
| SEQ ID | GI 1209857163 NM_015317 Homo sapiens | AATGGTAGTGGTCGA |
| NO: 1169 | pumilio RNA binding family member 2 | TATATCTCTGCAGCA |
| | (PUM2), transcript variant 1, mRNA | CCTGGAGCAGAAGCA |
| | | AAATATCGAAGTGCT |
| SEQ ID | GI 42716353 AY549314 Homo sapiens | TGATTAGTAATGTCT |
| NO: 1170 | tyrosine kinase 2 (TYK2) gene, | GCCACAGGCACAAGA |
| | complete cds | TTGACAAGAGGAGGG |
| | | GTGTATGCTGTGTAT |
| SEQ ID | GI 224809230 NG_011435 Homo sapiens | TGCTGAAACAGGATT |
| NO: 1171 | alcohol dehydrogenase 1B (class 1), | ATGAGAGTGACAGAC |
| | beta polypeptide (ADHIB), RefSeqGene | ACAGGACTGAAGACA |
| | on chromosome | GGGAGAAAATGTGCA |
| SEQ ID | GI 1047225988 NG_051241 Homo sapiens | TATTCTGGGCACCAA |
| NO: 1172 | arrestin domain containing 3 (ARRDC3), | AAGCACATGTAACTG |
| | RefSeqGene on chromosome 5 | CATACTGGCAACAGT |
| | | GAGACAAGCTAACAA |
| SEQ ID | GI 209977039 NG_008841 Homo sapiens | CTATGTCTCTCCTGT |
| NO: 1173 | dopamine receptor D2 (DRD2), | GCTTCTCTTTCATTT |
| | RefSeqGene on chromosome 11 | CCCCCCTGATGTCTT |
| | | GTGAATTCTCCCCTT |
| SEQ ID | GI 942429606 NG_008852 Homo sapiens | AGATTTGAAAGCTAT |
| NO: 1174 | insulin receptor (INSR), | TCTTGCAGGCAGGTG |
| | RefSeqGene on chromosome 19 | GAGAATCTTAAGGAT |
| | | CTCTGACACCCCAGT |
| SEQ ID | GI 256985185 NG_012797 Homo sapiens | AAGACCTAACGCCTA |
| NO: 1175 | membrane bound transcription factor | CTCAGAGTATCTGCC |
| | peptidase, site 2 (MBTPS2), | TGGGAAATGGAGAGA |
| | RefSeqGene on chromosc [symbol] | CCTCAACTTTTAGGA |
| SEQ ID | GI 291084732 NG_016743 Homo sapiens | CCCGGCAACAAGCAG |
| NO: 1176 | oxidized low density lipoprotein | AAGAAGCTTCACAGG |
| | receptor 1 (OLR1), RefSeqGene on | AGTCAGAAAACGAAC |
| | chromosome 12 | TCAAGGAAATGATAG |
| SEQ ID | GI 241896851 NG_012345 Homo sapiens | GGATAATCATCTCTT |
| NO: 1177 | peroxisome proliferator activated | GTATGGGCTGTTTGT |
| | receptor delta (PPARD), RefSeqGene | GCATTGTAGGATGTT |
| | on chromosome 6 | GAGCAGCATCCCTGG |
| SEQ ID | GI 334358873 NG_029029 HOMO SAPIENS | CCAAAAGTACATAAA |
| NO: 1178 | STEROL REGULATORY ELEMENT BINDING | AGACAATGACCATCA |
| | TRANSCRIPTION FACTOR 1 (SREBF1), | CCCAGTGCCGGTGAT |
| | REFSEQGENE ON CHROMOSO [symbol] | GCTAGAAGGAAAGGG |
| SEQ ID | GI 1049017896 NM_002192 Homo sapiens | TGCAGAAACAGTGTG |
| NO: 1179 | inhibin beta A subunit (INHBA), mRNA | AGAAATTAAGTCCTG |
| | | GTTCACTGCGCAGTA |
| | | GCAAAGATGGTCAAG |
| SEQ ID | GI 206597516 NM_001859 Homo sapiens | GGCAGTTGACCCACT |
| NO: 1180 | solute carrier family 31 member 1 | GGAATTTCTATAAGG |
| | (SLC31A1), mRNA | CTGGTACCCTTCCCA |
| | | GAGTTACAGAATCTT |
| SEQ ID | GI 83699688 DQ323999 Homo sapiens | AGTCTGTCTTGAGAT |
| NO: 1181 | TNF receptor-associated factor 4 | CTGATTTTTTCCCCC |
| | (TRAF4) gene, complete cds | TTTACCTAGCTGTGC |
| | | CCCCTCTGGTTATTT |
| SEQ ID | GI 197245396 NG_008399 Homo sapiens | GCCAGATATGGAAGC |
| NO: 1182 | aspartoacylase (ASPA), RefSeqGene | AGAATTGCTGACACT |
| | on chromosome 17 | TTGTTATCCAAACCA |
| | | AGAGGCCTGTCTTCA |
| SEQ ID | GI 307611940 NG_027759 Homo sapiens | CAGGCAATAAAATGC |
| NO: 1183 | caspase recruitment domain family | CAGCCAGGACACTGA |
| | member 11 (CARD11), RefSeqGene | AGAACACAGCAATGA |
| | on chromosome 7 | AACAAATTCCATCCA |
| SEQ ID | GI 323510659 NG_028143 Homo sapiens | AAAAAATCTGAAACT |
| NO: 1184 | interleukin 18 (IL18), RefSeqGene | GGTCAAGAAAGAACA |
| | on chromosome 11 | AGGAAGACTTGCCAA |
| | | AGCAAGGCCGGTGTG |
| SEQ ID | GI 237858755 NG_012119 Homo sapiens | ATGACAGAAAGAACT |
| NO: 1185 | protein tyrosine phosphatase, | CCAGAAATCTGCAAA |
| | non-receptor type 1 (PTPN1), | GGGATCACCTTGAGT |
| | RefSeqGene on chromosome 2 [symbol] | CTGGCTGGATACAGT |
| SEQ ID | GI 55742773 NM_001007033 Homo sapiens | ACAAGAGTTGAAAGA |
| NO: 1186 | C-type lectin domain containing | CATGTTTTCTAGATG |
| | 6A (CLEC6A), transcript variant 1, | GCTCACTCACATGGC |
| | mRNA | TGGCAACTTGGTGTT |
| SEQ ID | GI 1216866445 NM_005302 Homo sapiens | CTGTTGCTGTGAGGA |
| NO: 1187 | G protein-coupled receptor 37 (GPR37), | ATGCATTCAGAAGTC |
| | mRNA | TTCAACGGTGACCAG |
| | | TGATGACAATGACAA |
| SEQ ID | GI 153082754 NM_001547 Homo sapiens | GACAACCAGTACCTT |
| NO: 1188 | interferon induced protein with | AAAGTCCTCCTGGCT |
| | tetratricopeptide repeats 2 (IFIT2), | CTGAAGCTTCATAAG |
| | mRNA | ATGCGTGAAGAAGGT |
| SEQ ID | GI 12584865 AF307860 Homo sapiens | AGTCAGAGGGCAGAC |
| NO: 1189 | CYR61 protein (CYR61) gene, complete cds | CCTGTGAATATAACT |
| | | CCAGAATCTACCAAA |
| | | ACGGGGAAAGTTTCC |
| SEQ ID | GI 1121393451 NG_012066 Homo sapiens | GCATAGGTACCCATT |
| NO: 1190 | Fc fragment of IgG receptor IIa | AAGCCCATTGTGTTC |
| | (FCGR2A), RefSeqGene on chromosome 1 | ACTGGTTTCTTCCTG |
| | | CATTTGTGAGTCACT |
| SEQ ID | GI 1032528811 NG_050748 Homo sapiens | GGGGCAACGTTCACA |
| NO: 1191 | solute carrier family 4 member 3 | ATATTTATCAGCCTG |
| | (SLC4A3), RefSeqGene on chromosome 2 | TAAGGTGTAGCTGAT |
| | | CCAATTGGAATGGCT |
| SEQ ID | GI 156142193 NM_005880 Homo sapiens | TTTTCTCTCACATTT |
| NO: 1192 | DnaJ heat shock protein family | TTGGTGGGGGATTGT |
| | (Hsp40) member A2 (DNAJA2), mRNA | TCGGCTTCATGGGCA |
| | | ATCAGAGTAGAAGTC |
| SEQ ID | GI 541862110 NM_017434 Homo sapiens | CACCCAAGGTTTTAA |
| NO: 1193 | dual oxidase 1 (DUOX1), transcript | GCACGTTCTTTCATC |
| | variant 1, mRNA | AGACCCTGGCCCAAT |
| | | ACCTATGTATGCAAT |
| SEQ ID | GI 372466583 NM_005239 Homo sapiens | AACCATCTTAATAGC |
| NO: 1194 | ETS proto-oncogene 2, transcription | ATGCTGCTCCTTTTT |
| | factor (ETS2), transcript | GCTCAGTGTCCACAG |
| | variant 1, mRNA | CAAGATGACGTGATT |
| SEQ ID | GI 4755084 AFO17178 Homo sapiens | CAGAACACTATAGGT |
| NO: 1195 | pro alpha 1(1) collagen (COL1A1) | CACCTCCTTGCCCCA |
| | gene, complete cds | CACTGGAAATCTCAA |
| | | GCTTATCCATGTCTT |
| SEQ ID | GI 321267517 NG_028118 Homo sapiens | CAGTGCTGACCAATC |
| NO: 1196 | arrestin beta 1 (ARRB1), RefSeqGene | AAATTCCTCTACTGG |
| | on chromosome 11 | GAATTTGGAAGTGGG |
| | | CCATTCAACCAGTTA |
| SEQ ID | GI 315360647 NG_028023 Homo sapiens | TCTCTTAACCTTTAC |
| NO: 1197 | conserved helix-loop-helix ubiquitous | CTCATACAAGACCAG |
| | kinase (CHUK), RefSeqGene on | CTTGTCACTGCTCCT |
| | chromosome 10 | TATGCCTCAAACCCG |
| SEQ ID | GI 38327038 NM_002154 Homo sapiens | TTCAAAGCCCAAACC |
| NO: 1198 | heat shock protein family A | CAAAGTGGAACCTCC |
| | (Hsp70) member 4 (HSPA4), mRNA | AAAAGAGGAACAAAA |
| | | AAATGCAGAGCAGAA |
| SEQ ID | GI 156523967 NM_001618 Homo sapiens | AGTTGCCCTTGGAAA |
| NO: 1199 | poly(ADP-ribose) polymerase 1 | CATGTATGAACTGAA |
| | (PARP1), mRNA | GCACGCTTCACATAT |
| | | CAGCAAGTTACCCAA |
| SEQ ID | GI 1060099004 NM_001330400 Homo sapiens | CATGTCTGGGTCGTC |
| NO: 1200 | sideroflexin 5 (SFXN5), transcript | TTTTGAAAAGCTCAC |
| | variant 1, mRNA | TTCAAGCTCACCCTG |
| | | CTCCTACCTATCATA |
| SEQ ID | GI 338221701 NG_029188 Homo sapiens | ACATCTCCTCCTACT |
| NO: 1201 | caspase 9 (CASP9), RefSeqGene on | GTATATTTATTCTTG |
| | chromosome 1 | TGGGGAATGAGTCCT |
| | | GCCCTACGGGGAGTT |
| SEQ ID | GI 193211405 NG_008118 Homo sapiens | ACCTTGAGTAAATGG |
| NO: 1202 | left-right determination factor 2 | CTTTAAAATCCTCTC |
| | (LEFTY2), RefSeqGene on chromosome 1 | TCCACCATGTGCTCA |
| | | TCTGGAACAGGGGTA |
| SEQ ID | GI 294489300 NG_017057 Homo sapiens | TCATCTACTCCCAAG |
| NO: 1203 | solute carrier family 27 member 4 | AACAGGAAAACTCGG |
| | (SLC27A4), RefSeqGene on chromosome 9 | AGAAAAACAGTCCCA |
| | | TCAGATGTCCCCTTG |
| SEQ ID | GI 81230486 NM_001037162 Homo sapiens | CTTCTTTCTTGAAGG |
| NO: 1204 | acyl-CoA thioesterase 6 (ACOT6), mRNA | GCATCACAGCCACTG |
| | | TACTTATCAATGCCT |
| | | GTGTAGCCAACACAG |
| SEQ ID | GI 31563435 NM_144673 Homo sapiens | AGATCAAGATTCGGA |
| NO: 1205 | CKLF like MARVEL transmembrane domain | GTTTGGGCTGCCTAA |
| | containing 2 (CMTM2), transcript | TAGCTGCAATGATAC |
| | variant 1, mRNA | TGTTGTCCTCACTCA |
| SEQ ID | GI 260763875 NM_182569 Homo sapiens | AATCAAAGACTATTG |
| NO: 1206 | glycerophosphodiester phosphodiesterase | GATACATTTGGCATT |
| | domain containing 1 (GDPD1), | GGGCTGAGTGTGGTG |
| | transcript variant | GCTCATGCCTGTAAT |
| SEQ ID | GI 94557300 NM_006251 Homo sapiens | AATTATAACCGAGGG |
| NO: 1207 | protein kinase AMP-activated catalytic | CTGGCGTTTTGGAAT |
| | subunit alpha 1 (PRKAA1), transcript | GCAATTTGCACAGGG |
| | variant 1, mRNA | ATTGGAACATGATTT |
| SEQ ID | GI 109809760 NM_014445 Homo sapiens | TCAACTGACCTTGGG |
| NO: 1208 | stress associated endoplasmic | CCAGCAGGTTTTTAA |
| | reticulum protein 1 (SERP1), mRNA | CTAAATTGTTACTTG |
| | | CCTTTCTCACCCAGT |
| SEQ ID | GI 335057570 NG_029067 Homo sapiens | TTCCCACATTGGCCT |
| NO: 1209 | fibroblast growth factor 2 (FGF2), | ACTTGCTACCCCTCA |
| | RefSeqGene on chromosome 4 | AATACTCTGGATATG |
| | | ATCCTGTTGTAAGGC |
| SEQ ID | GI 1147544507 NG_028250 Homo sapiens | GTCTCTGTAATACAG |
| NO: 1210 | PPARG coactivator 1 alpha | TGTCCCAGAGGATGC |
| | (PPARGCIA), RefSeqGene on | TCCACAGTGCCGATA |
| | chromosome 4 | AACTTGGGTTAAGTG |
| SEQ ID | GI 22122007 AY135686 Homo sapiens | CTGCTTTCTGTGGAA |
| NO: 1211 | growth arrest and DNA-damage- | AAGATTCTAACTTGG |
| | inducible, alpha (GADD45A) gene, | TTCCCTGCCATCCCT |
| | complete cds | GAATACAAACGGGTT |
| SEQ ID | GI 1015539575 AH009651 Homo sapiens | CTGAAGCCCAGAATA |
| NO: 1212 | xeroderma pigmentosum group C | GCTACATGATGCCTC |
| | protein (XPC) gene, complete cds | TAAGGTCTTTTCCTT |
| | | GGTGAGCCTGAGAAT |
| SEQ ID | GI 255652911 NG_012637 Homo sapiens | GGAGAAACCCTTGAA |
| NO: 1213 | C-C motif chemokine receptor 5 | AAGACATCAAGCACA |
| | (gene/pseudogene) (CCR5), | GAAGGAGGAGGAGGA |
| | RefSeqGene on chromosome 3 | GGTTTAGGTCAAGAA |
| SEQ ID | GI 255652919 NG_012643 Homo sapiens | AGGCGGTTTGCACAA |
| NO: 1214 | carnitine palmitoyltransferase | CCCTCTAGTGCGTCG |
| | 1B (CPT1B), RefSeqGene on | TTTCCTCTTCTGTGA |
| | chromosome 22 | AATACTTTATAGGAT |
| SEQ ID | GI 388490119 NG_032755 Homo sapiens | TCCCGATTTGACAAA |
| NO: 1215 | interferon induced transmembrane | TGCCAGGAAAAGGAA |
| | protein 3 (IFITM3), RefSeqGene | ACTGTTGAGAAACCG |
| | on chromosome 11 | AAACTACTGGGGAAA |
| SEQ ID | GI 224809277 NG_011481 Homo sapiens | ATCTTTAAAGGCCAT |
| NO: 1216 | interleukin 18 receptor accessory | TGCTGATCCTGCCTT |
| | protein (IL18RAP), RefSeqGene on | GTTCCTTGGTTGCAG |
| | chromosome 2 | TCCTTGTAAGAAGAG |
| SEQ ID | GI 358679296 NG_031810 Homo sapiens | CAATTCCTACTCTGC |
| NO: 1217 | interferon regulatory factor 3 | TATTGGAAAAACAGG |
| | (IRF3), RefSeqGene on chromosome 19 | GGAGCAGGGAAGAGA |
| | | AAGCATTTTCCCCAG |
| SEQ ID | GI 1027250680 NG_047105 Homo sapiens | TATGAATGGGAAAGG |
| NO: 1218 | slit guidance ligand 2 (SLIT2), | AGAAGGTGGCATAAG |
| | RefSeqGene on chromosome 4 | TTCAGACCCAGAGTG |
| | | TGCTACAGTTCTTGG |
| SEQ ID | GI 341865593 NG_029549 Homo sapiens | CCCCTGAGATAGGTG |
| NO: 1219 | TGF-beta activated kinase 1 (MAP3K7) | CTACTGTTATTCCTA |
| | binding protein 1 (TAB1), RefSeqGene | TTTGACAGACTGAGA |
| | on chromosorr | CACTGAGGTGGTCGT |
| SEQ ID | GI 1043261406 NG_051107 Homo sapiens | TGTGATGGGCACACA |
| NO: 1220 | vacuole membrane protein 1 (VMP1), | CACAAACACACACAC |
| | RefSeqGene on chromosome 17 | AGTGCCTTAGCCATG |
| | | AGTAATATAAGCAAA |
| SEQ ID | GI 974005372 NM_005944 Homo sapiens | ATCTAGTCCAGTTCC |
| NO: 1221 | CD200 molecule (CD200), transcript | TGCTTACTGCTTTGC |
| | variant 1, mRNA | TAATAGCTGGCCTTG |
| | | CTAGAATCCTTGGTT |
| SEQ ID | GI 523425747 NM_004599 Homo sapiens | GTCAATCATAAACTG |
| NO: 1222 | sterol regulatory element binding | CGCCAGGAGAACATG |
| | transcription factor 2 (SREBF2), | GTGCTGAAGCTGGCA |
| | transcript variant 1, mRN [symbol] | AATCAAAAGAACAAG |
| SEQ ID | GI 24371240 NM_024630 Homo sapiens | CGTCATTCTTCGTTC |
| NO: 1223 | zinc finger DHHC-type containing | ACAGCAAACAGGATT |
| | 14 (ZDHHC14), transcript variant | CCTAAATGCCCTTAA |
| | 1, mRNA | GGACAGTCCTGCAAG |
| SEQ ID | GI 326535636 GU997628 Homo sapiens | GGCTGATTCAGATGC |
| NO: 1224 | annexin A1 (ANXAl) gene, complete cds | CAGGGTAAGGAAGTG |
| | | CTTACAAAATACTGC |
| | | TGCAGTTCATCTTCC |
| SEQ ID | GI 52352731 AY751746 Homo sapiens | ATTTCTGGCAGACAT |
| NO: 1225 | jun B proto-oncogene (JUNB) gene, | GTCTCCATCTTCTAC |
| | complete cds | CTGGCATATTTTACC |
| | | TGCCTCAGTGTACCC |
| SEQ ID | GI 221554534 NG_009447 Homo sapiens | CATGTCCCATGTGTC |
| NO: 1226 | dual oxidase 2 (DUOX2), RefSeqGene | TACCCAGATGCCAGA |
| | on chromosome 15 | GTAGGGATGTCAAGA |
| | | TACATGCAGTCATTC |
| SEQ ID | GI 270309111 NG_016167 Homo sapiens | AGAGGGTTTTTCTGT |
| NO: 1227 | glycogen synthase 2 (GYS2), | TGACAGCTCTGGATT |
| | RefSeqGene on chromosome 12 | GTTTGGGTAGATACT |
| | | GACAGGGCAGATACC |
| SEQ ID | GI 618857519 NG_034119 Homo sapiens | AGTGTGCCAATTGGG |
| NO: 1228 | nuclear receptor subfamily 2 | ACCAGTATCAAAAGA |
| | group F member 1 (NR2F1), | GAGCCTTGAAGAAAG |
| | RefSeqGene on chromosome 5 | AAAGAAAGGGCTTCC |
| SEQ ID | GI 237858740 NG_012111 Homo sapiens | TTGTACCTAAGTCAG |
| NO: 1229 | platelet derived growth factor | AGTTCATATGGGTCC |
| | subunit B (PDGFB), RefSeqGene | ATCACTTTTTAGCTG |
| | on chromosome 22 | GCAGAGCAGGTTCCC |
| SEQ ID | GI 189339276 NM_001204 Homo sapiens | GGACAGTACTATCTG |
| NO: 1230 | bone morphogenetic protein | GCCAAACAACCAACA |
| | receptor type 2 (BMPR2), mRNA | TAGTGACACATAGGG |
| | | CCCAAGAAATGTTGC |
| SEQ ID | GI 63054827 NM_001915 Homo sapiens | ATGACTAAGGAGCTG |
| NO: 1231 | cytochrome b561 (CYB561), | AAGAAGGCCCCCAGA |
| | transcript variant 1, mRNA | AGCGGATTTGTGGTG |
| | | GGTTATTTTATTTTG |
| SEQ ID | GI 899148489 NM_001135690 Homo sapiens | TCCTGGCTTGCGTAA |
| NO: 1232 | proenkephalin (PENK), mRNA | TGGAATGTGAAGGTA |
| | | AACTGCCTTCTCTGA |
| | | AAATTTGGGAAACCT |
| SEQ ID | GI 82583657 DQ297142 Homo sapiens | TCCAGTTCACTGACA |
| NO: 1233 | peroxiredoxin 1 (PRDX1) gene, | AACATGGGGAAGGTA |
| | complete cds | AGCCATCTGTCTGAT |
| | | AACAGGACAACAGAG |
| SEQ ID | GI 209977039 NG_008841 Homo sapiens | CACATAACAGAATGG |
| NO: 1234 | dopamine receptor D2 (DRD2), | GTTGATTCCATGTTA |
| | RefSeqGene on chromosome 11 | TGGTCTGCCCATAAG |
| | | TTGGGGCTTATGGCA |
| SEQ ID | GI 268370133 NG_015859 Homo sapiens | GTCTTAAGTTTGAGG |
| NO: 1235 | GATA binding protein 3 (GATA3), | GATCTGAGATTTCCC |
| | RefSeqGene on chromosome 10 | AGATTCCCGGCTGCA |
| | | CAGAATTTTCTTGCG |
| SEQ ID | GI 211064491 NG_008915 Homo sapiens | TCCTTCTCAAGCTAA |
| NO: 1236 | heat shock protein family D (Hsp60) | GAAAGGAAAACGGAA |
| | member 1 (HSPD1), RefSeqGene on | TTTCCCAATTCCTCT |
| | chromosome 2 | GGCTGCAGCTTTCAG |
| SEQ ID | GI 345525588 NG_029933 Homo sapiens | CAAAAGCTCATCCAT |
| NO: 1237 | interleukin 16 (IL16), RefSeqGene | TTGCGTTCCATGATG |
| | on chromosome 15 | CTGGGATTTACACTT |
| | | GAGGCTTAGCTTTGC |
| SEQ ID | GI 224809271 NG_011477 Homo sapiens | ATATCAGACACTTTC |
| NO: 1238 | TNF superfamily member 4 (TNFSF4), | CACCTTTGTTTTTGG |
| | RefSeqGene on chromosome 1 | ACAGCCCAGTGCTAA |
| | | AAGCACTGAAGGAGG |
| SEQ ID | GI 442092991JQ924061 Homo sapiens | GGATGGGATTGCAAA |
| NO: 1239 | ATG5 (ATG5) mRNA, complete cds | ATGACAGATTTGACC |
| | | AGTTTTGGGCCATCA |
| | | ATCGGAAACTCATGG |
| SEQ ID | GI 41393574 NM_033214 Homo sapiens | CACAGATCCAGGCCA |
| NO: 1240 | glycerol kinase 2 (GK2), mRNA | CAGAAAGTGAAATTC |
| | | GTTATGCCACATGGA |
| | | AGAAAGCCGTAATGA |
| SEQ ID | GI 440502998 JX512445 Homo sapiens | ATTGGCATTCTTTGT |
| NO: 1241 | integrin, alpha V (ITGAV) gene, | CTTGTGGTTCCTTCC |
| | complete cds | CTTATCTTCAAAGCC |
| | | AGCAATAGCTACCGG |
| SEQ ID | GI 226509596 NG_011798 Homo sapiens | GAAACCTTGGATGGA |
| NO: 1242 | ATP binding cassette subfamily C | CCAGAGGGAATACCT |
| | member 2 (ABCC2), RefSeqGene on | GGGTTCTGAAAAACT |
| | chromosome 10 | GAGCATAGTCACTGA |
| SEQ ID | GI 298104098 NG_021428 Homo sapiens | AAAATGAGAATCTGG |
| NO: 1243 | C-C motif chemokine receptor 2 | TTTGCTGGGGAAGAT |
| | (CCR2), RefSeqGene on chromosome 3 | AAAGGGTGTCGGAAA |
| | | ATGGCTGTTGGGTAA |
| SEQ ID | GI 1068937316 NG_051332 Homo sapiens | GCCAGGATTTGAACC |
| NO: 1244 | pyruvate dehyrogenase phosphatase | AAGTCCAAAGTCCCA |
| | catalytic subunit 2 (PDP2), | ATACTGGGCTTTCAG |
| | RefSeqGene on chromoso [symbol] | GAAGGGAGAATTATG |
| SEQ ID | GI 215598755 NG_009072 Homo sapiens | GCCTGTTAGGGCTGG |
| NO: 1245 | von Willebrand factor (VWF), | AACAGATCTTTATTT |
| | RefSeqGene on chromosome 12 | GTCTGGAATGCTTAA |
| | | GACCTCTCTTCCCCT |
| SEQ ID | GI 90577165 NM_032587 Homo sapiens | ATGCCAATTCAAGAG |
| NO: 1246 | caspase recruitment domain family | CCTGGGACTCAATGT |
| | member 6 (CARD6), mRNA | GAGCTCAGCCAGAAT |
| | | CTTCAGAATCTCTAT |
| SEQ ID | GI 387763637 NM_001098272 Homo sapiens | GATGAAGGAGTAGGA |
| NO: 1247 | 3-hydroxy-3-methylglutaryl-CoA | CTTGTGCATTCAAAC |
| | synthase 1 (HMGCS1), transcript | ATAGCAACTGAGCAT |
| | variant 1, mRNA | ATTCCAAGCCCTGCC |
| SEQ ID | GI 117606677 EF078888 Homo sapiens | TCAGCAGTGCTGAGG |
| NO: 1248 | Kruppel-like factor 2 | TTGAGACATCGCCTC |
| | (lung) (KLF2) gene, complete cds | TGGTTCCTAAAAGAT |
| | | CATTGCTCTTATCAG |
| SEQ ID | GI 3041866 U94788 Human p53 | GTTTGTTTCTTTGCT |
| NO: 1249 | (TP53) gene, complete cds | GCCGTGTTCCAGTTG |
| | | CTTTATCTGTTCACT |
| | | TGTGCCCTGACTTTC |
| SEQ ID | GI 190358485 NG_008035 Homo sapiens | TTTCATCATAGTCAG |
| NO: 1250 | carnitine palmitoyltransferase 2 | TCCAGTCCTGGGAAA |
| | (CPT2), RefSeqGene on chromosome 1 | CAAAATGGCATTGTT |
| | | AGCCTCACAACGCAG |
| SEQ ID | GI 344925888 NG_029692 Homo sapiens | ATACCTCAAGAATGA |
| NO: 1251 | 2′-5′-oligoadenylate synthetase 2 | ACGCCGAGGAAACTT |
| | (OAS2), RefSeqGene on chromosome 12 | CCCCCAGTAAAAACA |
| | | TCACCATTGCTTTCC |
| SEQ ID | GI 209447076 NG_008726 Homo sapiens | AAGTTTGTGGCTAGG |
| NO: 1252 | paraoxonase 3 (PON3), RefSeqGene | GACATGCCAATCCCT |
| | on chromosome 7 | TCTCCAGATGTGCTG |
| | | ACTTAAGGAGAATAA |
| SEQ ID | GI 597709817 NM_032965 Homo sapiens | GGATTCACCACTGCT |
| NO: 1253 | C-C motif chemokine ligand 15 | AACACCTCCTGGTTG |
| | (CCL15), mRNA | GAACTACAGGAATAG |
| | | AACTGGAAAGGGAAA |
| SEQ ID | GI 109637772 NM_015247 Homo sapiens | GCAAAGAAACTGAAG |
| NO: 1254 | CYLD lysine 63 deubiquitinase | GCAGAGTCCTAACGT |
| | (CYLD), transcript variant 1, mRNA | TGCATCTTATTCGAG |
| | | CTGGCAGTTCTGTTC |
| SEQ ID | GI 257743477 NM_181657 Homo sapiens | GAAGAGGAGATCCCC |
| NO: 1255 | leukotriene B4 receptor (LTB4R), | AAGCTGTCTACAGAT |
| | transcript variant 1, mRNA | CCTTACTGGAAGGTA |
| | | TGGTGCACCAATTTG |
| SEQ ID | GI 68160936 NM_005082 Homo sapiens | GTGATTCAGGGTCAT |
| NO: 1256 | tripartite motif containing 25 | GGTGACCCTGATCCA |
| | (TRIM25), mRNA | GTTTGGGTGGAAATC |
| | | CTTCCTAAGTATCAT |
| SEQ ID | GI 51537346 AY728138 Homo sapiens | GAAAGTGCAGAGTAG |
| NO: 1257 | chemokine (C-X-C motif) receptor 4 | ATGAGAACCTTTAAC |
| | (CXCR4) gene, complete cds | CACCAACATTAGGGA |
| | | GGGGTCCCAGACAAA |
| SEQ ID | GI 343098512 NG_029658 Homo sapiens | TGATTGGCATAAATG |
| NO: 1258 | BTB domain and CNC homolog 1 | GATCGTGATTCATCT |
| | (BACH1), RefSeqGene on chromosome 21 | TAGGTTGTGGCTGGG |
| | | GTCTACTTCCTTGGA |
| SEQ ID | GI 906847382 NG_042271 Homo sapiens | TTCTGATTGGTCCAT |
| NO: 1259 | calcium voltage-gated channel subunit | ATAGCTGAGCTCTGA |
| | alphal B (CACNA1B), RefSeqGene on | TTGGCTGAGGCAAGT |
| | chromosome 9 | GAACTCTGATTGGTT |
| SEQ ID | GI 994323652 NG_032159 Homo sapiens | GACAGGGTCGCTAGA |
| NO: 1260 | CASP2 and RIPK1 domain containing | CTCGGCTGGAATTAT |
| | adaptor with death domain (CRADD), | ATGTTTCTGTTCATC |
| | RefSeqGene on cl | ACATGGAAGGCTTGA |
| SEQ ID | GI 966209932 NG_033919 Homo sapiens | GCCTAAGTTGTTTCC |
| NO: 1261 | dedicator of cytokinesis 1 (DOCK1), | AAGGTAGCAAGGTTT |
| | RefSeqGene on chromosome 10 | TCCCATACTTTGAGC |
| | | CCCTCCCCATTTATC |
| SEQ ID | GI 324021728 NG_028155 Homo sapiens | GTGACTAAGTACATC |
| NO: 1262 | indoleamine 2,3-dioxygenase 1 (IDO1), | CTGATTCCTGCAAGC |
| | RefSeqGene on chromosome 8 | CAGCAGCCAAAGGAG |
| | | AATAAGACCTCTGAA |
| SEQ ID | GI 224809341 NG_011508 Homo sapiens | TGATTCTGAGCCCCT |
| NO: 1263 | insulin like growth factor binding | GAGGCAAAACGCTTT |
| | protein 3 (IGFBP3), RefSeqGene on | GCAGGTAATAATTCA |
| | chromosome 7 | GTTTTCCCATCTGAG |
| SEQ ID | GI 300116282 NG_023271 Homo sapiens | ATCACGGACATTACT |
| NO: 1264 | regenerating family member 1 alpha | CTGCTGTCCTTTTTC |
| | (REGIA), RefSeqGene on chromosome 2 | TGACCCGTCCTCTTG |
| | | GAGGACTCAGTATAT |
| SEQ ID | GI 597709817 NM_032965 Homo sapiens | ATAATAATAAAGAGA |
| NO: 1265 | C-C motif chemokine ligand 15 (CCL15), | CAAAAGAGGCCAGCC |
| | mRNA | ACCCACCTCCAACAC |
| | | CTCCTGTGAGTTTCT |
| SEQ ID | GI 55742773 NM_001007033 Homo sapiens | ACCTAGGTGAGCCCA |
| NO: 1266 | C-type lectin domain containing 6A | ATCATTCTGCAGAGC |
| | (CLEC6A), transcript variant 1, mRNA | AATGTGCTTCAATAG |
| | | TCTTCTGGAAACCTA |
| SEQ ID | GI 1049010878 AH003215 Homo sapiens | CCTTTCGGCGAGATA |
| NO: 1267 | CD8 antigen (CD8A) gene, complete cds, | CGTCTAACCCTGTGC |
| | alternatively spliced | AACAGCCACTACATT |
| | | ACTTCAAACTGAGAT |
| SEQ ID | GI 12584865 AF307860 Homo sapiens | TCAGAGACACACTCC |
| NO: 1268 | CYR61 protein (CYR61) gene, complete | TCTTCCCCGTTCTAC |
| | cds | TCTTTCAACAGATAA |
| | | CTTGCCTCTCACCTT |
| SEQ ID | GI 32815083 AY340073 Homo sapiens | CAGGAGGGGACGTAG |
| NO: 1269 | glutamate decarboxylase 2 (pancreatic | TATAGGGAGCAGGAA |
| | islets and brain, 65 kDa) (GAD2) | GTAAATAAAGCACCA |
| | gene, complete cds | GCAACTCAACAACTG |
| SEQ ID | GI 166706816 EU332867 Homo sapiens | CCCTGGGAAATGCAT |
| NO: 1270 | protein kinase C, epsilon (PRKCE) | ATGAATGAGTTGTGT |
| | gene, complete cds | GGTTGAGGAAGGAGA |
| | | GGCAAAGTCAATGTT |
| SEQ ID | GI 365777421 NG_031966 Homo sapiens | ACCAGCACTCCAGGA |
| NO: 1271 | interleukin 21 (IL21), RefSeqGene on | AAGGCAATGCATTGG |
| | chromosome 4 | AATAAACAAGATGAA |
| | | GCAGTGAGACTTGAA |
| SEQ ID | GI 212549771 NG_008990 Homo sapiens | TGATGAGAAGCAGTT |
| NO: 1272 | TNF superfamily member 11 (TNFSF11), | GTAACTCTTGGTCCT |
| | RefSeqGene on chromosome 13 | TTGCTGTCATCTGTG |
| | | GTGAAGTAGGCAGAG |
| SEQ ID | GI 209862753 NM_004343 Homo sapiens | AAATGAAGGACAAAC |
| NO: 1273 | calreticulin (CALR), mRNA | AGGACGAGGAGCAGA |
| | | GGCTTAAGGAGGAGG |
| | | AAGAAGACAAGAAAC |
| SEQ ID | GI 166706902 NM_002053 Homo sapiens | CTCATTGAGAACACT |
| NO: 1274 | guanylate binding protein 1 (GBP1), | AATGGGCGACTGATG |
| | mRNA | GCGAATCCAGAAGCT |
| | | CTGAAGATCCTTTCT |
| SEQ ID | GI 387598072 NM_005277 Homo sapiens | AGAGCTTCATGACAT |
| NO: 1275 | glycoprotein M6A (GPM6A), transcript | CCACTCTACTCGCTC |
| | variant 1, mRNA | CAAAGAGCGGCTCAA |
| | | TGCATACACATAAAT |
| SEQ ID | GI 20530138 AF261937 Homo sapiens | AAGTGAATCAGTGAA |
| NO: 1276 | NFKBIE protein (NFKBIE) gene, | GTTGGCCAGATATGA |
| | complete cds | GACCATGGTGTGGTG |
| | | AGGCCTATAAGAACC |
| SEQ ID | GI 260593639 NG_013007 Homo sapiens | GCCCCTGAACTTTCA |
| NO: 1277 | C-reactive protein (CRP), | GCCGAATACATCTTT |
| | RefSeqGene on chromosome 1 | TCCAAAGGAGTGAAT |
| | | TCAGGCCCTTGTATC |
| SEQ ID | GI 1121393451 NG_012066 Homo sapiens | CGGAAATCCGAACGC |
| NO: 1278 | Fc fragment of IgG receptor Iia | CAGCAGATCTGAATG |
| | (FCGR2A), RefSeqGene on chromosome 1 | GAATGTTCTGGATTG |
| | | AAGAAAGTGGGAAAT |
| SEQ ID | GI 1068937310 NG_008847 Homo sapiens | GTGGAGTTTCGGGCT |
| NO: 1279 | glucokinase (GCK), RefSeqGene on | GGACGGTGATTGGCT |
| | chromosome 7 | GAAACGAAGAAGTGT |
| | | TTAGAAAATCTCTTG |
| SEQ ID | GI 342837710 NG_029639 Homo sapiens | TTCTCACAGTGATCA |
| NO: 1280 | inhibitor of DNA binding 1, HLH | TTGGGCCATCTAGAT |
| | protein (ID1), RefSeqGene on | CACCTGGGGAGGTTG |
| | chromosome 20 | TTAAAATGCAGATGA |
| SEQ ID | GI 263191773 NG_015830 Homo sapiens | GAACTGGGTGAAGAA |
| NO: 1281 | insulin receptor substrate 1 (IRS1), | TGTGTCTCAAGATGG |
| | RefSeqGene on chromosome 2 | AAAAATGAATGGCTG |
| | | GTGCTTTAGCCTCCA |
| SEQ ID | GI 1028630729 NG_050573 Homo sapiens | AACTAGTACTGATGG |
| NO: 1282 | NLR family pyrin domain containing 6 | TGAAACAGGTCCATC |
| | (NLRP6), RefSeqGene on chromosome 11 | CTACGTTGATGGCAA |
| | | CTCGGCAAATTAGCT |
| SEQ ID | GI 31317227 NM_004430 Homo sapiens | AAAGGCAGGCTTCAA |
| NO: 1283 | early growth response 3 (EGR3), | CCAGCCACTCCAGCT |
| | transcript variant 1, mRNA | TTGTGTCTTCTCTCA |
| | | ATTACATAGCAATTT |
| SEQ ID | GI 32479526 NM_000218 Homo sapiens | GACGGTTCCTACACA |
| NO: 1284 | potassium voltage-gated channel | GGACAGGGGTTCCTT |
| | subfamily Q member 1 (KCNQ1), | CTGGGCATTACATCG |
| | transcript variant 1, Mrn [symbol] | CATAGAAATCAATAA |
| SEQ ID | GI 71648785 DQ147772 Homo sapiens | GAAGACCACAGACCA |
| NO: 1285 | apolipoprotein B mRNA editing enzyme, | TGGACTAGTTCTGAA |
| | catalytic polypeptide-like 3G | TGGTTTACAGACCTG |
| | (APOBEC3G) gene, comp [symbol] | GCAAATTTTGGTGCC |
| SEQ ID | GI 6537133 AF102137 Homo sapiens | GAGAAAAACCATGTT |
| NO: 1286 | PERI gene, complete cds | TCCAGGGAAGAGGTA |
| | | TCTGCAAACGAGATA |
| | | AGGCCAAAGCCACAG |
| SEQ ID | GI 169234676 NG_007118 Homo sapiens | TAGAGTTCTTGAAGA |
| NO: 1287 | ATP binding cassette subfamily B | TTCTCTTGGCTGCTG |
| | member 4 (ABCB4), RefSeqGene on | GCTGTGGGTACTTTA |
| | chromosome 7 | GGGCATCATTTCCTT |
| SEQ ID | GI 237820664 NG_012051 Homo sapiens | AAATAGCAAGAAAGT |
| NO: 1288 | caveolin 1 (CAV1), RefSeqGene on | GTCTCTGGCCCAAAG |
| | chromosome 7 | AGGCACATTCAATGA |
| | | GCTTGAAGGACAGTG |
| SEQ ID | GI 1143077144 NG_052854 Homo sapiens | GCCCAGGTACTATTG |
| NO: 1289 | gem nuclear organelle associated | TGTCCTTGTCCCTGT |
| | protein 5 (GEMIN5), RefSeqGene | GGGTCCTTCACTGTG |
| | on chromosome 5 | TACTCTAACAAATTC |
| SEQ ID | GI 209529670 NG_008721 Homo sapiens | CAGCTCAATGGCAGT |
| NO: 1290 | LIF, interleukin 6 family cytokine | GCCAATGCCCTCTTT |
| | (LIF), RefSeqGene on chromosome 22 | ATTCTCTATGTAAGT |
| | | TACCCCTGGGATACT |
| SEQ ID | GI 238018099 NG_012213 Homo sapiens | TCCCTTTTACCATCC |
| NO: 1291 | superoxide dismutase 3 (SOD3), | TGGGCATGTTGGCCT |
| | RefSeqGene on chromosome 4 | TATGTTCTCATTCTT |
| | | GTCATTGCATGGATC |
| SEQ ID | GI 281332104 NG_016228 Homo sapiens | ACACTTGTAATCATG |
| NO: 1292 | toll like receptor 1 (TLR1), | TACACAACTGGGAGT |
| | RefSeqGene on chromosome 4 | AGTGGGGATGAGTCT |
| | | GTGGGGAAAAACAGG |
| SEQ ID | GI 283837929 NG_016244 Homo sapiens | GCAATTAAGTGCGAA |
| NO: 1293 | toll like receptor 5 (TLR5), | AGTATGTGGCAGCAT |
| | RefSeqGene on chromosome 1 | CTGCTAGCTCAAAGA |
| | | AGACAGCTAGTGCAG |
| SEQ ID | GI 31657141 NM_181501 Homo sapiens | CAAATGGTTACCCTG |
| NO: 1294 | integrin subunit alpha 1 (ITGA1), mRNA | TGCTGTACCCAACTG |
| | | GATTGTCATCTTCTG |
| | | AGAATGCAAACTGCA |
| SEQ ID | GI 54781374 AY795972 Homo sapiens | TTCCACACGTAGAAA |
| NO: 1295 | matrix metalloproteinase 7 | CCCACTGTGTTTCTC |
| | (matrilysin, uterine) (MMP7) | TGGGTGAAGCACTCT |
| | gene, complete cds | CTATGCAAGGTTTAA |
| SEQ ID | GI 351542176 NG_030342 Homo sapiens | GGAAGAGACAGGAAT |
| NO: 1296 | collectin subfamily member 12 | GGGAAGGAGGTAGTG |
| | (COLEC12), RefSeqGene on | GTAACCATGAACGTT |
| | chromosome 18 | GCTGGAATATTCTGG |
| SEQ ID | GI 190358485 NG_008035 Homo sapiens | GAGATACCTCAGTGC |
| NO: 1297 | carnitine palmitoyltransferase 2 | ACAGAAGCCTCTCTT |
| | (CPT2), RefSeqGene on chromosome 1 | GAATGATGGCCAGTT |
| | | CAGGTAAACACTGAG |
| SEQ ID | GI 189217842 NG_007929 Homo sapiens | GCCAAAGGTAGAAAC |
| NO: 1298 | cytochrome P450 family 2 subfamily | GTGATATGGCACCAT |
| | B member 6 (CYP2B6), RefSeqGene | TGAGAACCTATAGGA |
| | on chromosome 19 | AGTCCAGTGAGCCTT |
| SEQ ID | GI 289666799 NG_016606 Homo sapiens | AGCCCCAGAGTCTAT |
| NO: 1299 | glycerol-3-phosphate dehydrogenase | GAATCAGGTCACTGG |
| | 2 (GPD2), RefSeqGene on chromosome 2 | AGTACAGTTTCTGTC |
| | | AAGTTCTCAGGTGAT |
| SEQ ID | GI 698320876 NM_015995 Homo sapiens | GACACCCCTTCCTGC |
| NO: 1300 | Kruppel like factor 13 (KLF13), | CGCCTTACTCTGTAC |
| | transcript variant 1, mRNA | ATAGATTTGCACTCT |
| | | GGAGTTTTCTGTTAG |
| SEQ ID | GI 594140701 NM_002661 Homo sapiens | GCTGCTGTGTAAAAT |
| NO: 1301 | phospholipase C gamma 2 (PLCG2), mRNA | CCATGCGTGGCCAAA |
| | | GAGAAGTCAGGGGAT |
| | | TATGACATAAATGGT |
| SEQ ID | GI 189217848 NG_007931 Homo sapiens | CATGCTAGAAGCTCA |
| NO: 1302 | cytochrome P450 family 2 subfamily J | CTCTCTTCCAGAGTG |
| | member 2 (CYP2J2), RefSeqGene on | TGAAGCAGATTGAAA |
| | chromosome 1 | GGGGTGACAGGATTT |
| SEQ ID | GI 1135567463 NG_052785 Homo sapiens | GGGGTAAACCTGGAA |
| NO: 1303 | galanin and GMAP prepropeptide (GAL), | TTTCGCGAGCTATCC |
| | RefSeqGene on chromosome 11 | AAAAGCACATGCATT |
| | | GTTCTAAGTCCTCTG |
| SEQ ID | GI 292658838 NG_016860 Homo sapiens | ACTGTCCAGGGAAGA |
| NO: 1304 | pyruvate dehydrogenase (lipoamide) beta | CTAGACTCACTCATG |
| | (PDHB), RefSeqGene on chromosome 3 | AAGTGTTAAGTTCCC |
| | | CATCAGTAGAGCGTG |
| SEQ ID | GI 694879461 NG_031891 Homo sapiens | GTATTCCCTGCTTCA |
| NO: 1305 | sodium voltage-gated channel alpha | GCCAGTTTTCAGCTT |
| | subunit 10 (SCN10A), RefSeqGene on | TCTTAGGTCTGCTCT |
| | chromosome 3 | GTCAGTTACCACTCA |
| SEQ ID | GI 281332105 NG_016229 Homo sapiens | TCTGTGTTAGTCTGG |
| NO: 1306 | toll like receptor 2 (TLR2), | AGGGTGAACCTATTT |
| | RefSeqGene on chromosome 4 | TAATGCAGGAGGGCA |
| | | TAGACTTTTGGGTGG |
| SEQ ID | GI 62953114 NM_012342 Homo sapiens | AAGGTGAAATTCGAT |
| NO: 1307 | BMP and activin membrane bound | GCTACTGTGATGCTG |
| | inhibitor (BAMBI), mRNA | CCCACTGTGTAGCCA |
| | | CTGGTTATATGTGTA |
| SEQ ID | GI 323422857 NM_001565 Homo sapiens | GGAACCTCCAGTCTC |
| NO: 1308 | C-X-C motif chemokine ligand 10 | AGCACCATGAATCAA |
| | (CXCL10), mRNA | ACTGCCATTCTGATT |
| | | TGCTGCCTTATCTTT |
| SEQ ID | GI 2909349 Y12863 Homo sapiens | GACCAACCTTCCATT |
| NO: 1309 | mRNA for growth factor FIGF | CACACCAGCTAAGGA |
| | | GTCCCTGGTTCATTG |
| | | ATGGATGTCTTCTAG |
| SEQ ID | GI 169791020 NM_005328 Homo sapiens | AATCAGCCACTTATA |
| NO: 1310 | hyaluronan synthase 2 (HAS2), mRNA | TCTGGAAGAACAACT |
| | | TCCACGAAAAGGGTC |
| | | CCGGTGAGACAGATG |
| SEQ ID | GI 164695749 AK315559 Homo sapiens | TTCCTACTGGATGTT |
| NO: 1311 | cDNA, FLI96630, Homo sapiens | CTCTCAATAATTGTG |
| | methionyl aminopeptidase 2 (METAP2), mRNA | CTGCCCATTATACTC |
| | | CCAATGCCGGTGACA |
| SEQ ID | GI 32815083 AY340073 Homo sapiens | ATTGCAAAGTTGGGA |
| NO: 1312 | glutamate decarboxylase 2 (pancreatic | GGAGACAGCAGAAAA |
| | islets and brain, 65 kDa) (GAD2) gene, | GGTCATTGCAACCTG |
| | complete cds | TTTCTGCTCTACTTT |
| SEQ ID | GI 1101010097 NG_052015 Homo sapiens | GTCTGCATGTCTCTT |
| NO: 1313 | adenosine deaminase, RNA specific B1 | CCTTCCGGTACTCTG |
| | (ADARB1), RefSeqGene on chromosome 21 | TCTTATGAAATCTGG |
| | | CTGACTTGGTTTCCC |
| SEQ ID | GI 1078569903 NG_051800 Homo sapiens | CCCTCCCACATTTCT |
| NO: 1314 | dedicator of cytokinesis 2 (DOCK2), | GCTGTGTGCCAACTT |
| | RefSeqGene on chromosome 5 | GGAAACTTTCTTCAA |
| | | GATTCTCAACTGTCA |
| SEQ ID | GI 237858713 NG_012098 Homo sapiens | GACTCCTTAGCTTAG |
| NO: 1315 | KIT ligand (KITLG), RefSeqGene on | TGAGGCCATTGGTCC |
| | chromosome 12 | TTGTTTGAACTCTGT |
| | | CTTCCACACCTCAAT |
| SEQ ID | GI 176866166 NG_007524 Homo sapiens | CCTGAGCACCAGACA |
| NO: 1316 | KRAS proto-oncogene, GTPase (KRAS), | TGTATGTATATCAAG |
| | RefSeqGene on chromosome 12 | ATGCCTGCAGGTCAT |
| | | ATCCACCAGGACAAC |
| SEQ ID | GI 210032137 NG_008855 Homo sapiens | CCAGAGCGTCAGTAC |
| NO: 1317 | lipoprotein lipase (LPL), RefSeqGene | TGAGAGGACACTAAG |
| | on chromosome 8 | CATGTGACCTTCACT |
| | | ACTCCTGTTCTGAAT |
| SEQ ID | GI 1147623323 NG_029437 Homo sapiens | AATTTTCCATGCATT |
| NO: 1318 | mitogen-activated protein kinase | TCCATTCAGCGGATC |
| | kinase 6 (MAP2K6), RefSeqGene on | CGAGCCACAGTAAAT |
| | chromosome 17 | AGCCAGGAACAGAAA |
| SEQ ID | GI 269954733 NG_016014 Homo sapiens | TTATAAGAATCACGT |
| NO: 1319 | membrane spanning 4-domains A2 | GTCCAGAACACTCTG |
| | (MS4A2), RefSeqGene on chromosome 11 | ATTCACAGCCAAGGA |
| | | TCCAGAAGGCCAAGG |
| SEQ ID | GI 336088631 NG_029118 Homo sapiens | TCCTCTACCCAGGTC |
| NO: 1320 | RAR related orphan receptor C (RORC), | TCCGGTGTGCATTTG |
| | RefSeqGene on chromosome 1 | GGCGTCTTATCCTCA |
| | | TAAAGTATATAGCTT |
| SEQ ID | GI 241666450 NG_012339 Homo sapiens | TGCAGAGCGCACGTG |
| NO: 1321 | succinate dehydrogenase complex | TTGGATTCTGCCTGG |
| | flavoprotein subunit A (SDHA), | TAAGAGTTTTTCCCA |
| | RefSeqGene on chromoso | TATGATAGCAAAAAA |
| SEQ ID | GI 83699682 DQ314888 Homo sapiens | CAAGTGTAGGAGGAC |
| NO: 1322 | v-rel reticuloendotheliosis viral | TAACAGGCACTGTAG |
| | oncogene homolog (avian) (REL) | CAAACTAAAGAGCCC |
| | gene, complete cds | TGAATAAATGGGGCT |
| SEQ ID | GI 30039656 AY273786 Homo sapiens | GTCACATTCTGCTGT |
| NO: 1323 | tumor necrosis factor, alpha-induced | TGGGGAAACTGAGGC |
| | protein 2 (TNFAIP2) gene, complete cds | ACAGAAAATGGTAGG |
| | | GATTTCATTCACGAG |
| SEQ ID | GI 224809353 NG_011513 Homo sapiens | CAAAGTCCCTTGAGC |
| NO: 1324 | ATP binding cassette subfamily B | GCTTGGTATTTGGAT |
| | member 1 (ABCB1), RefSeqGene on | CTCTCATTACCACGC |
| | chromosome 7 | AATCCATGAGCATTC |
| SEQ ID | GI 291084732 NG_016743 Homo sapiens | TCAGGTGGCCAACTA |
| NO: 1325 | oxidized low density lipoprotein | TGGATTTCTTTCCTC |
| | receptor 1 (OLR1), RefSeqGene on | TCTGTGCCTCAATAA |
| | chromosome 12 | AATAGAGGCCAGGAC |
| SEQ ID | GI 307746866 NG_027761 Homo sapiens | GTTCAGAGTGAGAGA |
| NO: 1326 | heat shock protein family A (Hsp70) | GTTGGAATCTTCTTT |
| | member 5 (HSPA5), RefSeqGene on | CTCAGAAGCACACGT |
| | chromosome 9 | CACTGGCCATGGAAT |
| SEQ ID | GI 1000232109 NG_046737 Homo sapiens | TCCCCATTTCATGAG |
| NO: 1327 | protein phosphatase 1 regulatory subunit | AGGGGATGACTGTAC |
| | 15B (PPP1R15B), RefSeqGene on chromosome 1 | ACACGTCAGTGTGTA |
| | | TTAGGCCATCAAAAC |
| SEQ ID | GI 219879808 NM_000922 Homo sapiens | GTTGCCTTTCTGAGG |
| NO: 1328 | phosphodiesterase 3B (PDE3B), mRNA | GATTTCTGCTCAATG |
| | | CAATACACTGTTCAG |
| | | TGCTATTCTCCCAGC |
| SEQ ID | GI 115298685 NM_006099 Homo sapiens | TTCTTACATCCAGAG |
| NO: 1329 | protein inhibitor of activated STAT 3 | AGGTTCTGCCAGGAG |
| | (PIAS3), mRNA | CCAAATGTGATTATA |
| | | CCATACAGGTGCAGC |
| SEQ ID | GI 166706752 EU332835 Homo sapiens | ATTGCTTCAGTTCTA |
| NO: 1330 | v-akt murine thymoma viral oncogene | ACAGGCCGCAAAACT |
| | homolog l (AKT1) gene, complete cds | GATACCGCTTTTCGG |
| | | GAAATACACGAAACA |
| SEQ ID | GI 327412387 NG_028268 Homo sapiens | TTCTGCTGTATAGGC |
| NO: 1331 | ATP binding cassette subfamily C member 1 | AAAAGCAGAAGGTAA |
| | (ABCC1), RefSeqGene on chromosome 16 | GAATGTTCTGGGCAG |
| | | TGGGAAATGCATGTG |
| SEQ ID | GI 341865614 NG_029559 Homo sapiens | AATTTCTTTTCCCTG |
| NO: 1332 | chordin (CHRD), RefSeqGene on chromosome 3 | GACATCATACCTGCT |
| | | GCTGAAATCCAACAG |
| | | GATGCTTGCTTTCCT |
| SEQ ID | GI 224282141 NG_009873 Homo sapiens | TTATAGCCAGACTGG |
| NO: 1333 | CREB binding protein (CREBBP), RefSeqGene | ACTTGTGTATCTGCT |
| | on chromosome 16 | GTGATCACACACACC |
| | | CGTTTCACCATTCTG |
| SEQ ID | GI 683524074 NG_034246 Homo sapiens | CCTCTCCTGTAAAAT |
| NO: 1334 | lipase E, hormone sensitive type (LIPE), | GGGGATGCTGGCAAT |
| | RefSeqGene on chromosome 19 | AATAGAGATTTGAAC |
| | | CTAGGTGGTGTGGGT |
| SEQ ID | GI 256985185 NG_012797 Homo sapiens | CAGGGCTAAGGTAGG |
| NO: 1335 | membrane bound transcription factor | GAGCTCTGATGGTAG |
| | peptidase, site 2 (MBTPS2), RefSeqGene | CATTTGCTGGTTTCT |
| | on chromosc | GTGGTGTAAATACTT |
| SEQ ID | GI 341865593 NG_029549 Homo sapiens | TGCCTTCCCGGTATG |
| NO: 1336 | TGF-beta activated kinase 1 (MAP3K7) | CCCTATTTCTCTCTG |
| | binding protein 1 (TAB1), RefSeqGene | TGTGTAGTCTTTGCT |
| | on chromoso [symbol] | TAGCTGTTCACATTC |
| SEQ ID | GI 305410826 NG_027551 Homo sapiens | CTGAGGATGGGTCTG |
| NO: 1337 | transient receptor potential cation | AGGAATTCGAGACCA |
| | channel subfamily M member 4 (TRPM4), | TAGTTTTGAAGGCCC |
| | RefSeqGene or [symbol] | TTGTGAAGGGTAAAA |
| SEQ ID | GI 117606531 EF064740 Homo sapiens | GGATTTGTTTATTTC |
| NO: 1338 | Epstein-Barr virus induced gene 3 | CTGTCTTCCCCCTGA |
| | (EBI3) gene, complete cds | TGGATTGTGAGTTCC |
| | | ATGAGGACAGGGGTA |
| SEQ ID | GI 221139797 NG_009359 Homo sapiens | ACCTCAGTCTTCCCA |
| NO: 1339 | chloride voltage-gated channel Ka | TCTTTAAAATGGGGT |
| | (CLCNKA), RefSeqGene on chromosome 1 | GGTTACTGGGAAGGC |
| | | AGAGAAGGAAGAAAG |
| SEQ ID | GI 224809291 NG_011495 Homo sapiens | GACGTCTTGGATAAG |
| NO: 1340 | interferon induced with helicase C | TGCATGGAGGAGGAA |
| | domain 1 (IFIH1), RefSeqGene on | CTGTTGACAATTGAA |
| | chromosome 2 | GACAGAAACCGGGTA |
| SEQ ID | GI 342307076 NG_029608 Homo sapiens | GTTTTACAGATGTGA |
| NO: 1341 | phospholipase D2 (PLD2), | AAAAGCAGGCACTGA |
| | RefSeqGene on chromosome 17 | GAGGTAAGGTAATTT |
| | | GCCCAAGGTCACGCA |
| SEQ ID | GI 393537030 NM_000151 Homo sapiens | CCAGAAAGATTCAGA |
| NO: 1342 | glucose-6-phosphatase catalytic | GTTGGCCAAGAATGA |
| | subunit (G6PC), transcript | ACATTGGCTACCAGA |
| | variant 1, mRNA | CCACAAGTCAGCATG |
| SEQ ID | GI 662033918 NM_014625 Homo sapiens | TCCTTGTGCAAACCA |
| NO: 1343 | NPHS2, podocin (NPHS2), transcript | CTATGAAGCGTCTCC |
| | variant 1, mRNA | TAGCACATCGATCCC |
| | | TCACTGAAATTCTTC |
| SEQ ID | GI 156071494 NM_002787 Homo sapiens | CATGACTGCTTCTAG |
| NO: 1344 | proteasome subunit alpha 2 | AGCTGGGAGGATCTG |
| | (PSMA2), mRNA | CAGACTTTCACGGAT |
| | | TCATGGAACAAGAAA |
| SEQ ID | GI 57165045 AY864066 Homo sapiens | TGCAAAATCGAGGAC |
| NO: 1345 | cyclin B2 (CCNB2) gene, complete cds | ATTGATAACGAAGAT |
| | | TGGGAGAACCCTCAG |
| | | CTCTGCAGTGACTAC |
| SEQ ID | GI 1049010846 AH002874 Homo sapiens | GCTGGATTTTTGCAC |
| NO: 1346 | MC-CPA gene, complete sequence; | ATCCAAAGATACTGC |
| | and carboxypeptidase A (CPA3) | TCCCTGAAGCAGCCA |
| | gene, complete cds | CTTAACAAGTGAGAA |
| SEQ ID | GI 1049011196 AH002836 Homo sapiens | GACAACAAATGTAAG |
| NO: 1347 | 1g J-chain gene, complete cds; | TGTGCCCGGATTACT |
| | and IGJ gene, complete sequence | TCCAGGATCATCCGT |
| | | TCTTCCGAAGATCCT |
| SEQ ID | GI 342349316 NG_029618 Homo sapiens | TTGTGCCCTATTACT |
| NO: 1348 | CD28 molecule (CD28), RefSeqGene | TAACCTTGAGGGACA |
| | on chromosome 2 | TAGAGAGCATGAGAC |
| | | ACCAAGGGGCTTTTG |
| SEQ ID | GI 1025608659 NG_047042 Homo sapiens | TTCCTCACGTTACTC |
| NO: 1349 | cell division cycle 42 (CDC42), | ATATAATTACCGTGT |
| | RefSeqGene on chromosome 1 | GATCGTGTGGTGCTC |
| | | TGCTGCTTGTTGGTC |
| SEQ ID | GI 1028908925 NG_050621 Homo sapiens | TCAAAGTCCAATCAC |
| NO: 1350 | glutathione peroxidase 4 (GPX4), | GGTGACTCAGTTCCC |
| | RefSeqGene on chromosome 19 | AAGTATGAGATGAGT |
| | | GTTTCTGTTGCCACA |
| SEQ ID | GI 336285218 NG_029143 Homo sapiens | CCACGCTCACAACCC |
| NO: 1351 | mitogen-activated protein kinase | TCCAGTGATTTCCAT |
| | kinase 5 (MAP2K5), RefSeqGene on | CTCACTCCAAAGTAA |
| | chromosome 15 | AAGACCAAGTCCTTA |
| SEQ ID | GI 342307060 NG_029601 Homo sapiens | CAACGTGAAAACTGG |
| NO: 1352 | Pim-1 proto-oncogene, serine/threonine | TTAAATCAAGCGCAC |
| | kinase (PIM1), RefSeqGene on | CCTCCCACCCTCGTT |
| | chromosome 6 | TTAGATGAGGAATTT |
| SEQ ID | GI 588480539 NG_033255 Homo sapiens | TGAGAGGTATTTAAC |
| NO: 1353 | ras homolog family member H (RHOH), | ATGTCAGTGGGAAGG |
| | RefSeqGene on chromosome 4 | AATTTCTATTCACTG |
| | | CAGTGGGCTGTCCCC |
| SEQ ID | GI 33333853 AF546700 Homo sapiens | GGACTTCAATTTCTT |
| NO: 1354 | androgen regulated a/b hydrolase II | CACCTCTAAAATGGG |
| | (ABHD2) mRNA, complete cds | GGACTTGGACCAGGT |
| | | AGATTGCTGAGCTCA |
| SEQ ID | GI 21885466 AY128534 Homo sapiens | CATTGACTTTGTGGT |
| NO: 1355 | cyclin-dependent kinase 6 (CDK6) | GGCAACACAGTCAGT |
| | gene, complete cds | TCTAGACAGCTTTGC |
| | | TGGGAGGAAAATCAA |
| SEQ ID | GI 7637751 AF250378 Homo sapiens | TTGACCACGAGTCTT |
| NO: 1356 | E2F transcription factor 4 (E2F4) | CTTCTGTAGGTCTTG |
| | gene, complete cds | TGTCCCTGTCTCCTG |
| | | TGGTGACTAATTTCA |
| SEQ ID | GI 90657247 DQ449938 Homo sapiens | ATGTCTCCAGGGGCA |
| NO: 1357 | Fas (TNFRSF6)-associated via death | CCAAAGAATGCATTT |
| | domain (FADD) gene, complete cds | TCCACGTTTGTCCCT |
| | | AAACGTTAATGCATA |
| SEQ ID | GI 88698111 DQ388429 Homo sapiens | GTCAGGCCCTACCAT |
| NO: 1358 | heat shock 70 kDa protein 1B (HSPA1B) | TGAGGAGGTGGATTA |
| | gene, complete cds | GGGGCCTTTGTTCTT |
| | | TAGTATGTTTGTCTT |
| SEQ ID | GI 237874185 NG_012167 Homo sapiens | CCAGTTTCCCTGGTG |
| NO: 1359 | CD209 molecule (CD209), RefSeqGene | TAAATATTCCCATGG |
| | on chromosome 19 | TGGCTGATTTCAAGC |
| | | TGTCGTTGTGATGTC |
| SEQ ID | GI 301336145 NG_023427 Homo sapiens | TAAGCTGATGTTTGA |
| NO: 1360 | myocyte enhancer factor 2C (MEF2C), | TTTAATTGTGAGTTC |
| | RefSeqGene on chromosome 5 | CTGGGGCCTCCAGGC |
| | | CTCCTTCATCTGTAA |
| SEQ ID | GI 541862110 NM_017434 Homo sapiens | CATTTTGGGACATTC |
| NO: 1361 | dual oxidase 1 (DUOX1), transcript | TAATCCCTGAGCCCC |
| | variant 1, mRNA | TATTATTTTCATCAT |
| | | GGGCTTCTGCCTGGC |
| SEQ ID | GI 209977115 NM_153692 Homo sapiens | TGGTGATGTGATTGG |
| NO: 1362 | HtrA serine peptidase 4 (HTRA4), | CGTCAATTCATTGAG |
| | mRNA | GGTGACTGATGGAAT |
| | | CTCCTTTGCAATTCC |
| SEQ ID | GI 157909838 NM_006252 Homo sapiens | AGCTGGAACATTGTT |
| NO: 1363 | protein kinase AMP-activated | ACAGCAGGCTTTTTG |
| | catalytic subunit alpha 2 (PRKAA2), | TTGCTCATGGGCAGA |
| | mRNA | TAGAGGGAAAGAATC |
| SEQ ID | GI 27979118 AY219866 Homo sapiens | TGCCATGCTGAAACA |
| NO: 1364 | caspase 3, apoptosis-related cysteine | GTATGCCGACAAGCT |
| | protease (CASP3) gene, complete cds | TGAATTTATGCACAT |
| | | TCTTACCCGGGTTAA |
| SEQ ID | GI 56788327 AY851164 Homo sapiens | CATGGCTAGCCCAAG |
| NO: 1365 | carboxylesterase 2 (intestine, liver) | AATCCGACATTTCCC |
| | (CES2) gene, complete cds | CCATTTCACAGACGA |
| | | GCAAATTGAAGTTAG |
| SEQ ID | GI 345441766 NG_029866 Homo sapiens | TTTCCACATGGTTAG |
| NO: 1366 | DnaJ heat shock protein family (Hsp40) | TTGATCTGATGTTTC |
| | member A3 (DNAJA3), RefSeqGene on | TGCCAGGGGCATGAG |
| | chromosome | TTTTGGAGAGTCCTA |
| SEQ ID | GI 215820636 NG_009079 Homo sapiens | CCAACCATGCTTTAC |
| NO: 1367 | malonyl-CoA decarboxylase (MLYCD), | AGAGAGAGTTTCCTC |
| | RefSeqGene on chromosome 16 | ACCTTGGGGTGTTTT |
| | | CAAGTCTGTCACCTA |
| SEQ ID | GI 207444685 NG_008651 Homo sapiens | CTGGGAGCATGAGGG |
| NO: 1368 | NLRfamily pyrin domain containing 12 | TAGATAAGCTGGATG |
| | (NLRP12), RefSeqGene on chromosome 19 | GACAAATACGTTATG |
| | | CACTGGTGTATGCAA |
| SEQ ID | GI 1068937316 NG_051332 Homo sapiens | CCGTGTCTATTGGCA |
| NO: 1369 | pyruvate dehyrogenase phosphatase | GGAACTGCTTGATTT |
| | catalytic subunit 2 (PDP2), | GCACATGGAAATGGG |
| | RefSeqGene on chromosoi | ACTAAGCATTGAAGA |
| SEQ ID | GI 260898757 NM_021571 Homo sapiens | CATTCAGTGGGTGCA |
| NO: 1370 | caspase recruitment domain | GGCACAATAAATGCC |
| | family member 18 (CARD18), mRNA | TTGCTGGATTGCCTA |
| | | TTAGAGGATGAAGTT |
| SEQ ID | GI 325652102 NM_002811 Homo sapiens | GACCAGTGAAATTGG |
| NO: 1371 | proteasome 26S subunit, non-ATPase 7 | AGCAGAGGAAGCTGA |
| | (PSMD7), mRNA | GGAAGTTGGAGTTGA |
| | | ACACTTGTTACGAGA |
| SEQ ID | GI 385198082 NM_005226 Homo sapiens | CTTGTCCACTTTGGG |
| NO: 1372 | sphingosine-1-phosphate receptor 3 | CTCCAGAGTCTTTCA |
| | (S1PR3), mRNA | GATGTACTAAGCTGC |
| | | TGACATCATCCACTG |
| SEQ ID | GI 117606555 EF064752 Homo sapiens | TCTAACCCAAGAGTC |
| NO: 1373 | CD5 molecule (CD5) gene, complete cds | AGAGATGGGGCTGGT |
| | | CATGTTCCTTTGGTT |
| | | TGAATAACTCCCTTG |
| SEQ ID | GI 262331523 NG_013339 Homo sapiens | AGAATTAGGTGATAG |
| NO: 1374 | catalase (CAT), RefSeqGene on | AGGCTACATCCAGGA |
| | chromosome 11 | GCTGCCCATGGCAGG |
| | | ATTGTATCTCCTATC |
| SEQ ID | GI 339515631 NG_029366 Homo sapiens | ATTTCAAACGCCACT |
| NO: 1375 | coagulation factor III, tissue factor | TCCACCAGAAGGCCT |
| | (F3), RefSeqGene on chromosome 1 | TGCTACTGCACCAAC |
| | | TAGTTACTATCTCTT |
| SEQ ID | GI 319803083 NG_011853 Homo sapiens | TCATATTCACCTAGG |
| NO: 1376 | galactosylceramidase (GALC), | GCCATGTGCTTACCT |
| | RefSeqGene on chromosome 14 | AGTTAAATGCTTTTC |
| | | CTGCACCACTGGACA |
| SEQ ID | GI 161086967 NG_007121 Homo sapiens | CATTGACTCTGCAAA |
| NO: 1377 | hydroxyacyl-CoA dehydrogenase/ | ATACTGCCACTTAGC |
| | 3-ketoacyl-CoA thiolase/enoyl-CoA | TGGCACTGTGATGGA |
| | hydratase (trifunctiona [symbol] | GTCTTCAGTAGTCCA |
| SEQ ID | GI 341865570 NG_029565 Homo sapiens | TGATCAGCCTACTGC |
| NO: 1378 | interleukin 24 (IL24), RefSeqGene | CTCCTTTCCCAATAA |
| | on chromosome 1 | GGTCATGTTCTTCTC |
| | | TTCCTCTGCTCCTAT |
| SEQ ID | GI 937500889 NG_032896 Homo sapiens | TTCTCTTCCTCTGTC |
| NO: 1379 | purinergic receptor P2Y1 (P2RY1), | CCCCAAATACTGTTC |
| | RefSeqGene on chromosome 3 | AGGCACTATTTCTCC |
| | | TCCTGTACTTCCCTC |
| SEQ ID | GI 1005261235 NM_000399 Homo sapiens | AAGACAAGCAAAACT |
| NO: 1380 | early growth response 2 (EGR2), | GATGTGCACTTTATG |
| | transcript variant 1, mRNA | GCTTGGGACTGATTT |
| | | GGGGGACATTGTACA |
| SEQ ID | GI 118722343 NM_030949 Homo sapiens | AATAAACTTGTGGTT |
| NO: 1381 | protein phosphatase 1 regulatory | GTCCTTTAACTGGAG |
| | inhibitor subunit 14C (PPP1R14C), mRNA | GTCCCAGCACATGTG |
| | | TTTTCAAGAGGCCAC |
| SEQ ID | GI 109809760 NM_014445 Homo sapiens | GTTACTGTGGTTTCA |
| NO: 1382 | stress associated endoplasmic | CTAAAAGCTACCGTG |
| | reticulum protein 1 (SERP1), mRNA | GTGGAGTGAAGTCAG |
| | | TCAGGGAAGGTTTGT |
| SEQ ID | GI 154482086 EU048234 Homo sapiens | TGTGTGTACCAATGC |
| NO: 1383 | CCAAT/enhancer binding protein | GGGCTGGGGCATTTT |
| | (C/EBP), alpha (CEBPA) gene, | CGAGAAATTACACAT |
| | complete cds | TCCTTTTACACTCTG |
| SEQ ID | GI 292494880 NG_007148 Homo sapiens | CCTGTCTGACTCCTA |
| NO: 1384 | ATPase phospholipid transporting 8B1 | CATGTCCTGTAGGTC |
| | (ATP8B1), RefSeqGene on chromosome 18 | TCAGGTTAGACCTCA |
| | | ATTTCTGCAACACCT |
| SEQ ID | GI 334848115 NG_029039 Homo sapiens | GACCTTTGCTCTGAA |
| NO: 1385 | major histocompatibility complex, | TGACATGGGGAGTTA |
| | class 1, G (HLA-G), RefSeqGene on | GAGGACAGTTTTGGA |
| | chromosome 6 | AAGTGGGACATGGTA |
| SEQ ID | GI 514825583 NG_033271 Homo sapiens | CGCATCCGGGAAGGA |
| NO: 1386 | Kruppel like factor 10 (KLF10), | GTAGAATCAGCAATT |
| | RefSeqGene on chromosome 8 | CTAGTTTTGGATATG |
| | | TTGTCTGAGGAGCCC |
| SEQ ID | GI 359751452 NG_031884 Homo sapiens | GCTCCATCGATCCCT |
| NO: 1387 | mitogen-activated protein kinase | TCACATTTGTCTCCT |
| | kinase kinase 1 (MAP3K1), RefSeqGene | GGTTTACGAGATGTG |
| | on chromosome 5 | GCTCTTCGTTGTTTA |
| SEQ ID | GI 281332104 NG_016228 Homo sapiens | GCAATGATCAACAAA |
| NO: 1388 | toll like receptor 1 (TLR1), | GCCTGAGTGATCTTG |
| | RefSeqGene on chromosome 4 | GCCCTAACTCCTCTC |
| | | ATCTTCCCTAACTCC |
| SEQ ID | GI 209447071 NM_005639 Homo sapiens | AGAGCAAGAGAAATT |
| NO: 1389 | synaptotagmin 1 (SYT1), transcript | GGGTGATATCTGCTT |
| | variant 1, mRNA | CTCCCTTCGCTACGT |
| | | ACCTACTGCTGGTAA |
| SEQ ID | GI 1049010711 AH003036 Homo sapiens | CTAAGCATCCACTTC |
| NO: 1390 | Bruton agammaglobulinemia (BTK) | TTCAGGGACCCTCAA |
| | gene, complete cds | GGGGTGATACGTCAT |
| | | TATGTTGTGTGTTCC |
| SEQ ID | GI 46241839 AY588246 Homo sapiens | TTGATGAGGTGTCCC |
| NO: 1391 | epidermal growth factor receptor | TCATCCTCTCTTAGC |
| | (erythroblastic leukemia viral | TGAAAATCAGCAGCT |
| | (v-erb-b) oncogene homolog, a [symbol] | GCCTCAACTTCTTTT |
| | | C |
| SEQ ID | GI 190570161 NG_008053 Homo sapiens | CCTTACTCCCTCCTT |
| NO: 1392 | acyl-CoA synthetase long chain | CTGTTGTACCTAATG |
| | family member 4 (ACSL4), RefSeqGene | TCCATGAGTCCCGAG |
| | on chromosome X | TCTCTGCTTCAGTTT |
| SEQ ID | GI 344030205 NG_029797 Homo sapiens | TTTTCACTGAGTTGA |
| NO: 1393 | glutaredoxin (GLRX), RefSeqGene | AGCCACAGCATTTTC |
| | on chromosome 5 | TGAGAAATGGAGAGG |
| | | ACACCCGTTCTTTGC |
| SEQ ID | GI 222144288 NG_009492 Homo sapiens | CTGAAGTCTTGACTG |
| NO: 1394 | insulin like growth factor 1 receptor | ACTCAGTACCTGCCC |
| | (IGF1R), RefSeqGene on chromosome 15 | CTCCCTCCATCTCTG |
| | | AAATGTGGAGAATTA |
| SEQ ID | GI 270483830 NG_016181 Homo sapiens | TCAGGAGATAGCTTG |
| NO: 1395 | sphingosine-1-phosphate receptor 1 | AGAATGCAATTTTTA |
| | (SIPR1), RefSeqGene on chromosome 1 | GGGCCTCCTTGGAGA |
| | | GCAATGGGAAGAGAC |
| SEQ ID | GI 815930042 NM_001308319 Homo sapiens | AATCCATCCCCAAAA |
| NO: 1396 | chromodomain helicase DNA binding | CCAACCCAATGGTTT |
| | protein 9 (CHD9), transcript | GTTTCCAGATGTATC |
| | variant 1, mRNA | AGATGGCAGTCCAAT |
| SEQ ID | GI 181979 M29366 Human epidermal | ATCAGTATGGAGGAG |
| NO: 1397 | growth factor receptor (ERBB3) | TTTGTGTAGCCAGCT |
| | mRNA, complete cds | GTCCCCATAACTTTG |
| | | TGGTGGATCAAACAT |
| SEQ ID | GI 208879434 NM_004895 Homo sapiens | AAAAGAGATGAGCCG |
| NO: 1398 | NLRfamily pyrin domain containing 3 | AAGTGGGGTTCAGAT |
| | (NLRP3), transcript variant 1, mRNA | AATGCACGTGTTTCG |
| | | AATCCCACTGTGATA |
| SEQ ID | GI 23955951 AF549495 Homo sapiens | TGGAACAAGAAGAGA |
| NO: 1399 | cyclin G2 (CCNG2) gene, complete cds | GATTCCAACCTCGAG |
| | | AAAAAGGGCTGAGTT |
| | | TGATTGAGGCTACCC |
| SEQ ID | GI 117606491 EF064720 Homo sapiens | GGGGAAGGGAATGGA |
| NO: 1400 | interleukin 27 (IL27) gene, complete cds | ATAGGGTTAGGAATG |
| | | AGGTTGGAGATGGGA |
| | | TGGAAAGATGGAAAG |
| SEQ ID | GI 170560906 EU567146 Homo sapiens | CGCTTACCCCATGTT |
| NO: 1401 | solute carrier family 22 (organic anion | TCCCAAATGAGGAAT |
| | transporter), member 6 (SLC22A6) | CCAAGGCCAAGAAGA |
| | gene, complete cds | AGGGAAAGGATTTAC |
| SEQ ID | GI 389565477 NG_032771 Homo sapiens | GCATATACAGTGTTC |
| NO: 1402 | bone morphogenetic protein 7 (BMP7), | TCCAGCTGACTTATC |
| | RefSeqGene on chromosome 20 | TCGGAGACTGGTCCT |
| | | GTCAGCCGTTAAAGA |
| SEQ ID | GI 211904170 NG_008937 Homo sapiens | GGCAGAGGGATGAGA |
| NO: 1403 | CD44 molecule (Indian blood group) | ATCTTCGTAGACCAT |
| | (CD44), RefSeqGene on chromosome 11 | ACAAACCCAGCCCCA |
| | | CATTGTTATGTAAGG |
| SEQ ID | GI 334848119 NG_029043 Homo sapiens | GCAGATATTGGGAAA |
| NO: 1404 | Fc fragment of IgE receptor Ig (FCER1G), | GAGAAGGGGTAGTGA |
| | RefSeqGene on chromosome 1 | GTAATCATGAATGGC |
| | | GAGGGTCTTGAAGGC |
| SEQ ID | GI 325053739 NG_028167 Homo sapiens | AGCTAAGGATTGGAA |
| NO: 1405 | growth differentiation factor 3 (GDF3), | TGACAACCCCCGGAA |
| | RefSeqGene on chromosome 12 | AAATTTCGGGTTATT |
| | | CCTGGAGATACTGGT |
| SEQ ID | GI 255652922 NG_012646 Homo sapiens | TCTGAGGATAGCACT |
| NO: 1406 | transforming growth factor beta | GTGGTTCTGTGACAG |
| | induced (TGFBI), RefSeqGene on | ATAATGAAGGATTTG |
| | chromosome 5 | GAAGCAGGAATGCCC |
| SEQ ID | GI 1040975354 NM_001737 Homo sapiens | AAGCACACCTTTTGA |
| NO: 1407 | complement C9 (C9), mRNA | CAATGAGTTCTACAA |
| | | TGGACTCTGTAACCG |
| | | GGATCGGGATGGAAA |
| SEQ ID | GI 666637974 NM_005922 Homo sapiens | GAAGGAGCTCTTCCA |
| NO: 1408 | mitogen-activated protein kinase | TGAAGCCAGAGAAAG |
| | kinase kinase 4 (MAP3K4), | GGCTTCCAAAGCACT |
| | transcript variant 1, mRNA | TGGATTTGCTAAAAT |
| SEQ ID | GI 297374833 NM_006184 Homo sapiens | GCAGGATCCCAATGT |
| NO: 1409 | nucleobindin 1 (NUCB1), mRNA | ACAGGTGGATCATCT |
| | | GAATCTCCTGAAACA |
| | | GTTTGAACACCTGGA |
| SEQ ID | GI 195222745 NM_018290 Homo sapiens | TAATTCGTGGTCTTA |
| NO: 1410 | phosphoglucomutase 2 (PGM2), mRNA | CAGCCTTCCAAAATA |
| | | ACTCCAGTTGGGCAC |
| | | CCATGAGCTAGGATC |
| SEQ ID | GI 21914799 AY131997 Homo sapiens | TGAAGTAACCTGCCA |
| NO: 1411 | tumor necrosis factor receptor | ATTCCCAATCAAAAA |
| | superfamily, member 1A (TNFRSF1A) | CACATCCTTTCAACA |
| | gene, complete cds | TCTGCCCTGTGTCCA |
| SEQ ID | GI 163954918 NG_007084 Homo sapiens | TTCTCTCTCTCAGCT |
| NO: 1412 | apolipoprotein E (APOE), RefSeqGene | TTGTCTCTCTCTCTT |
| | on chromosome 19 | CCCTTCTGACTCAGT |
| | | CTCTCACACTCGTCC |
| SEQ ID | GI 345478654 NG_029871 Homo sapiens | TTAGTCTTTTCCCAC |
| NO: 1413 | activating transcription factor 3 | TTCTACTGCCTACAA |
| | (ATF3), RefSeqGene on chromosome 1 | TACCCAATCAGTTCC |
| | | TTCCCACCCCAATGC |
| SEQ ID | GI 1024846760 NG_046988 Homo sapiens | TCTGAAAGAGGAGAT |
| NO: 1414 | E2F transcription factor 1 (E2F1), | CTGCATAAATGAGAA |
| | RefSeqGene on chromosome 20 | AATCGGGAAAGGCGT |
| | | TCTGGAATGGGTGGT |
| SEQ ID | GI 284813593 NG_016440 Homo sapiens | GCATCTGCTCCAAAA |
| NO: 1415 | MAF bZIP transcription factor (MAF), | CTGCTTTGAGTTACT |
| | RefSeqGene on chromosome 16 | CAGAACTTCAACCTC |
| | | CCAATGCACTGAAGG |
| SEQ ID | GI 237858770 NG_012124 Homo sapiens | AGAGCAAGCAAGTGC |
| NO: 1416 | selectin E (SELE), RefSeqGene | AGTGTTTATTAACCA |
| | on chromosome 1 | AGGTGTGGTGTGGTA |
| | | TTGTGAACGAGCTTG |
| SEQ ID | GI 1147566 X80590 H. sapiens PHKGI mRNA | CAGGGCACCCCAACA |
| NO: 1417 | | TCATACAGCTGAAGG |
| | | ACACTTATGAGACCA |
| | | ACACTTTCTTCTTCT |
| SEQ ID | GI 80861465 NM_014331 Homo sapiens | GAGTCCATTTAGCCC |
| NO: 1418 | solute carrier family 7 member 11 | AGTGGGAAAGTCTTG |
| | (SLC7A11), mRNA | GAACTCAGGTTACCC |
| | | TTGAAGGATATGCTG |
| SEQ ID | GI 163954918 NG_007084 Homo sapiens | TCTTGCTGCTGAACC |
| NO: 1419 | apolipoprotein E (APOE), RefSeqGene | ACGGGTTTCTCCTCT |
| | on chromosome 19 | GAAACATCTGGGATT |
| | | TATAACAGGGCTTAG |
| SEQ ID | GI 299116025 NG_023038 Homo sapiens | GGCTTCCACGCAAGC |
| NO: 1420 | autophagy related 16 like 1 (ATG16L1), | ATAATCTAGCTTTGT |
| | RefSeqGene on chromosome 2 | GGTTCCTCATAGGAC |
| | | ACATGCAGAAAATGG |
| SEQ ID | GI 696221409 NG_041768 Homo sapiens | AAAACTCCGATCTGC |
| NO: 1421 | CD24 molecule (CD24), RefSeqGene on | CTTTGTTCCATGACA |
| | chromosome 6 | AAGGAGAGATTATCA |
| | | CGTGCCCTACTGGAC |
| SEQ ID | GI 225007613 NG_011612 Homo sapiens | GTAGAGACTGGTGTG |
| NO: 1422 | upstream transcription factor 1 (USF1), | AGGTTAGATGTATGC |
| | RefSeqGene on chromosome 1 | TGAAACCTCTGTGTG |
| | | GGGAAAGAAGGGAGA |
| SEQ ID | GI 974005408 NM_015133 Homo sapiens | GAATGGGCAAAGAAG |
| NO: 1423 | mitogen-activated protein kinase 8 | TGGGGAATCTGCTAC |
| | interacting protein 3 (MAPK8IP3), | TGGAAAACTCACAGC |
| | transcript variant 1, [symbol] | TTCTGGAAACCAAAA |
| SEQ ID | GI 1145587777 KX533498 Homo sapiens | AAGCTGCTTTTCACC |
| NO: 1424 | macrophage receptor (MARCO) | AAATTGCAATGGAGC |
| | mRNA, complete cds | CTTTCGAAATCAATG |
| | | TTCCAAAGCCCAAGA |
| SEQ ID | GI 1036032720 AH002608 Homo sapiens | CTTCACAGAAATGGG |
| NO: 1425 | amphiregulin (AREG) gene, complete cds | TGGGAAGGGGCTCTT |
| | | GATTGAGATAGAAGC |
| | | CCATCCTACATGAAG |
| SEQ ID | GI 306482660 NG_027673 Homo sapiens | GCAGACACAAAGACA |
| NO: 1426 | activating transcription factor 1 | GTTCATACCCTCTTG |
| | (ATF1), RefSeqGene on chromosome 12 | TAGGTTTTTCTCCAA |
| | | GGACCTCCAGCAGTT |
| SEQ ID | GI 906847382 NG_042271 Homo sapiens | TTTGCTCAGCCCAAC |
| NO: 1427 | calcium voltage-gated channel subunit | CCTAACCCTAACTCC |
| | alphal B (CACNA1B), RefSeqGene on | TACCAGCCAATTCAA |
| | chromosome 9 | GGTGATGATTAGATG |
| SEQ ID | GI 334085262 NG_029008 Homo sapiens | AGTGCTCTCCTTATT |
| NO: 1428 | CRK proto-oncogene, adaptor protein | CTCCTCCTGCAGGTC |
| | (CRK), RefSeqGene on chromosome 17 | GGTGAGCTGGTAAAG |
| | | GTTACGAAGATTAAT |
| SEQ ID | GI 1159261282 NG_052987 Homo sapiens | GCTTTCTGTTGCTAG |
| NO: 1429 | zinc finger protein 148 (ZNF148), | ATTGGTGGTTGGGAA |
| | RefSeqGene on chromosome 3 | ATGCCAGGTCTGGAT |
| | | TGTCTTCTTCTGTTG |
| SEQ ID | GI 30841527 AY277591 Homo sapiens | ATATGGAGCTCTCAT |
| NO: 1430 | GalNAc-T5 (GALNT5) mRNA, complete cds | TCAAGGTGTGGATGT |
| | | GTGGTGGTGAAATTG |
| | | AGATCATTCCCTGCT |
| SEQ ID | GI 27369416 AY166858 Homo sapiens | GGCAGGAACAGTTCT |
| NO: 1431 | TGS1 mRNA, complete cds | TTAGCTGTCGGCGTA |
| | | AATGACCAAGTTCAT |
| | | GACCTATCAAGGTCT |
| SEQ ID | GI 117606529 EF064739 Homo sapiens | CACAATTCCACCTGC |
| NO: 1432 | Fas ligand (TNF superfamily, | ATCTGCATGATCTCT |
| | member 6) (FASLG) gene, complete cds | TCCTTCCACCTGGTC |
| | | TCAGTGGAGAAATAG |
| SEQ ID | GI 27357272 AY195619 Homo sapiens | ACCAAGAGCAGAGAC |
| NO: 1433 | interleukin 10 receptor, alpha | AGAGAGCAAGGACCG |
| | (IL10RA) gene, complete cds | AAACTGCTTCTTCAT |
| | | GACACCAAAATACAA |
| SEQ ID | GI 14278713 AF377330 Homo sapiens | TGTCTGTGCTAGGAT |
| NO: 1434 | urokinase-type plasminogen activator | ATAGAACTTGGAGAA |
| | (PLAU) gene, complete cds | TGGAGCCTTGGGATG |
| | | GATTCCAGCCTAACT |
| SEQ ID | GI 1028630732 NG_050570 Homo sapiens | CTGTGGGGTCATGAT |
| NO: 1435 | carnitine palmitoyltransferase 1C | TGAGTTTGAATCCTG |
| | (CPT1C), RefSeqGene on chromosome 19 | ACTCCACCGCTTAGT |
| | | AGCTGTGTCATCTTG |
| SEQ ID | GI 283945491 NG_016362 Homo sapiens | ACAGCTTCAGCGATG |
| NO: 1436 | C-X3-C motif chemokine receptor 1 | TCAGATTTTTTATGT |
| | (CX3CR1), RefSeqGene on chromosome 3 | AAATGGTGCCTTCAC |
| | | ATGCTCTGGGGCATT |
| SEQ ID | GI 212549728 NG_008977 Homo sapiens | ATTTGGAAAAGGCTG |
| NO: 1437 | methylmalonyl-CoA epimerase (MCEE), | CAGCATTTTATAAGA |
| | RefSeqGene on chromosome 2 | ATATTCTGGGGGCCC |
| | | AGGTAAGTGAAGCGG |
| SEQ ID | GI 229577384 NG_011990 Homo sapiens | ACAACCAAGAACTTT |
| NO: 1438 | SMAD family member 3 (SMAD3), | TCCCCACCAGGAGTG |
| | RefSeqGene on chromosome 15 | GGTATATTTCAACTT |
| | | CTGTAATCCCTGCGT |
| SEQ ID | GI 227908772 NG_011947 Homo sapiens | TTTAAGCACATGGAC |
| NO: 1439 | tryptophan hydroxylase 1 (TPH1), | TGTGGAGCTGGACAG |
| | RefSeqGene on chromosome 11 | ACTAGATTTGAATTC |
| | | CGGCTCTGACACTTG |
| SEQ ID | GI 111161293 NM_005746 Homo sapiens | TGGTATTTGGCCATC |
| NO: 1440 | nicotinamide phosphoribosyltransferase | TCCATTCTGAGCACC |
| | (NAMPT), mRNA | AAACGGTTAACACGA |
| | | ATGTCCACTAGAACT |
| SEQ ID | GI 152112977 EU012027 Homo sapiens | GGTACAACACGCCTC |
| NO: 1441 | high-mobility group box 1 (HMGB1) | CTGAAAGGGTCTGAT |
| | gene, complete cds | AGCTTTCAGTAGCAG |
| | | TAAGACACTTGCATG |
| SEQ ID | GI 302128095 AB542701 Homo sapiens | GCTCACCATTGTATC |
| NO: 1442 | NOD2 gene for nucleotide-binding | TTCTTTTCCAGGTTG |
| | oligomerization domain containing 2, | TCCAATAACTGCATC |
| | | ACCTACCTAGGGGCA |
| SEQ ID | GI 262331523 NG_013339 Homo sapiens | TGTTCACTGGACCTG |
| NO: 1443 | catalase (CAT), RefSeqGene on | CATCTTAACTCTGAG |
| | chromosome 11 | GCTGGCATTGTTAAA |
| | | CACCTGTAGTACTGC |
| SEQ ID | GI 1024846796 NG_046983 Homo sapiens | ACCTCTAATTTGTTT |
| NO: 1444 | cAMP responsive element binding | CCTGATTCCCAGTAC |
| | protein 3 (CREB3), RefSeqGene on | CGGCTTGGACATTCA |
| | chromosome 9 | TCCCTTTCCTAGCAC |
| SEQ ID | GI 335892864 NG_029105 Homo sapiens | GTTGTTGACAAAAAC |
| NO: 1445 | interleukin 1 receptor accessory | GAAAAGAGCAACTGG |
| | protein (ILIRAP), RefSeqGene on | CTCTGGGCTAGTGGG |
| | chromosome 3 | GGAAGAAAGAAATCA |
| SEQ ID | GI 169808406 NG_007450 Homo sapiens | TTGTTCACCATTGCA |
| NO: 1446 | leptin (LEP), RefSeqGene on chromosome 7 | TCCTCGGTGCTGAGC |
| | | ACTGCGTCTGACATA |
| | | TTATTTAGAAGGTCA |
| SEQ ID | GI 289802979 NG_016616 Homo sapiens | GAGTTGGTGATAGCC |
| NO: 1447 | phosphorylase kinase catalytic subunit | TGAGCAAGGAAATCC |
| | gamma 2 (PHKG2), RefSeqGene | TTGTCTAGGACTTTG |
| | on chromosome 16 | TGTGTGCCTCATGAA |
| SEQ ID | GI 224809271 NG_011477 Homo sapiens | CAGAGCATTGCCCTT |
| NO: 1448 | TNF superfamily member 4 (TNFSF4), | AAGCACGAAGGGCAC |
| | RefSeqGene on chromosome 1 | TAATTAGCTATCACA |
| | | CCACGAAGAGTACAG |
| SEQ ID | GI 806607 M90696 Homo sapiens | ATGGAAACAAAGGCT |
| NO: 1449 | cathepsin S (CTSS) mRNA, complete | GCAATGGTGGCTTCA |
| | cds | TGACAACGGCTTTCC |
| | | AGTACATCATTGATA |
| SEQ ID | GI 476704 L26336 Homo sapiens | GGGGAAAACAAGCTG |
| NO: 1450 | heat shock protein (HSPA2) gene, | CTCGAGCTTTATTTC |
| | complete cds | TTCGGTGCAACCAAC |
| | | TCAGAATGAATTCCT |
| SEQ ID | GI 225735573 NG_011718 Homo sapiens | ACTTAGTTCCACCTG |
| NO: 1451 | alcohol dehydrogenase 1C (class 1), | CAGAGATTCTCATTT |
| | gamma polypeptide (ADHIC), | AATTGGCATGGGCTA |
| | RefSeqGene on chromosoi | TGACCTGGACTGTGG |
| SEQ ID | GI 238018066 NG_012196 Homo sapiens | CAGTATGTCTTCTCC |
| NO: 1452 | fibronectin 1 (FN1), RefSeqGene | TATCTCTATCTCCCC |
| | on chromosome 2 | TCCAAATTCTCCACC |
| | | CTCACTTGCAGCCTG |
| SEQ ID | GI 207440658 NG_008647 Homo sapiens | TTCAGTCCACACTTC |
| NO: 1453 | GTP cyclohydrolase 1 (GCH1), | TAGTAGGTTGAGTTC |
| | RefSeqGene on chromosome 14 | AGCCCAAGATTAAGG |
| | | CTCTGGTCTGGAGTC |
| SEQ ID | GI 584613557 NG_033959 Homo sapiens | AGCTGGCCTGTTACT |
| NO: 1454 | poly(ADP-ribose) polymerase 2 | CTTAGGGAACTATCT |
| | (PARP2), RefSeqGene on chromosome 14 | TATGTGTGGCATTTC |
| | | CTTTGCAGTTGGGAA |
| SEQ ID | GI 1000232109 NG_046737 Homo sapiens | CAGAAGATTGGCTTT |
| NO: 1455 | protein phosphatase 1 regulatory | GGGTTTCTAGTGATG |
| | subunit 15B (PPP1R15B), RefSeqGene | CTCTGTTGTCTCCAC |
| | on chromosome 1 | CAGATGGCGTTATCT |
| SEQ ID | GI 974987499 NG_023044 Homo sapiens | GTGACCCGTTATGTT |
| NO: 1456 | radixin (RDX), RefSeqGene on chromosome 11 | TTAGCTTAGTGCATG |
| | | CACTAGGAGGTCACC |
| | | ATCAGTGTTTGACCT |
| SEQ ID | GI 6272556 AF123887 Homo sapiens | GCTGATATGTGCCTG |
| NO: 1457 | EROIL (EROIL) mRNA, | TTTCTGGACAATGGA |
| | | GGCGAAAGAGTGGAA |
| | | TTTCATTCAAAGGCA |
| SEQ ID | GI 167614499 NM_002286 Homo sapiens | CACCTACAGAGATGG |
| NO: 1458 | lymphocyte activating 3 (LAG3), mRNA | CTTCAACGTCTCCAT |
| | | CATGTATAACCTCAC |
| | | TGTTCTGGGTCTGGA |
| SEQ ID | GI 570359552 NM_001288718 Homo sapiens | TTGGCTTCACTAGAA |
| NO: 1459 | signal transducer and activator of | TCCCCAGTTCTTCCC |
| | transcription 5A (STAT5A), | CTCTCTACAGTTTTG |
| | transcript variant 1, mRNA | TCTCTGAGGTCACAA |
| SEQ ID | GI 4455117 AF125158 Homo sapiens | CACATGTGGTGAAGT |
| NO: 1460 | zinc finger DNA binding protein 99 | CATAGTTAAAGGAGC |
| | (ZNF281) mRNA, complete cds | CACTAGTGCAGAACC |
| | | TGGGTCATCAAACCA |
| SEQ ID | GI 224809231 NG_011436 Homo sapiens | ATCACGGCAAGCCTT |
| NO: 1461 | alpha 2-HS glycoprotein (AHSG), | CTTTTAGGGTGTCAC |
| | RefSeqGene on chromosome 3 | CTCATCCCTTTAAGA |
| | | GCTGTCATCAAATCA |
| SEQ ID | GI 300068920 NG_023233 Homo sapiens | ACTTGGTTTCTTTGC |
| NO: 1462 | bone morphogenetic protein 2 (BMP2), | TCAGAGCCGTGTCCC |
| | RefSeqGene on chromosome 20 | ATTTAGGAAAGTACT |
| | | AGGAGTTTGGGGTTC |
| SEQ ID | GI 648216408 NG_034146 Homo sapiens | ACACCATTCCTCATA |
| NO: 1463 | phosphoglucomutase 3 (PGM3), | GTCTACAGGAAATCA |
| | RefSeqGene on chromosome 6 | GGTGGGGAAAGCAAA |
| | | GTCAGGAAATGGCAT |
| SEQ ID | GI 353409895 NG_030365 Homo sapiens | CTGCCAACACACTGT |
| NO: 1464 | POU class 2 homeobox 2 (POU2F2), | GTCATTTTGCCCAAG |
| | RefSeqGene on chromosome 19 | TTGCTTTCTGTCTCT |
| | | GGACTTCCTTTTACA |
| SEQ ID | GI 212549771 NG_008990 Homo sapiens | CTATGAATGTGAAGT |
| NO: 1465 | TNF superfamily member 11 (TNFSF11), | GAGCTTGGCCAACCT |
| | RefSeqGene on chromosome 13 | GCTCTTCCTCCAGAG |
| | | TGTTTCATATCAGCA |
| SEQ ID | GI 807201065 NM_001008397 Homo sapiens | TGTTAGCGAAGAAAA |
| NO: 1466 | glutathione peroxidase 8 (putative) | CAGAAGCCACCAACA |
| | (GPX8), transcript variant 1, mRNA | AGCCTATCTGTCCTT |
| | | TGCAATTTTGCTCCA |
| SEQ ID | GI 802084027 NM_006236 Homo sapiens | AGAGAGAGAGAGTCC |
| NO: 1467 | POU class 3 homeobox 3 (POU3F3), mRNA | CCTTTCCTTTCACTT |
| | | TCTCCCTCCAAAACA |
| | | GCTGCCTAAAGAGAT |
| SEQ ID | GI 24756896 L47234 Homo sapiens | CAGGTGAAAAAGATG |
| NO: 1468 | ERCC2 (ERCC2) and kinesin light chain | AGGCACAGAGAGGCT |
| | (KLC2) genes, complete cds, complete | GAGTAACTTGTCTTA |
| | sequence | GGTCACACAGCCAAG |
| SEQ ID | GI 117606495 EF064722 Homo sapiens | CCAATCCCCTTTTCA |
| NO: 1469 | interleukin 6 signal transducer (gpl30 | TCTCCCCTGAAATTC |
| | , oncostatin M receptor) (II6ST) gene, | TATTCGCTCCGCACT |
| | complete cds | TGTCTTCATTCTCGA |
| SEQ ID | GI 166706778 EU332848 Homo sapiens | TGTCTTCACTCCTGG |
| NO: 1470 | integrin, beta 5 (ITGB5) gene, | GAGGAGTCCATTTTT |
| | complete cds | GTTAATTTTCTCAGT |
| | | GACTGGGAAGGGCTG |
| SEQ ID | GI 33867482 AY368173 Homo sapiens | AAGGGAAAGGGCGTT |
| NO: 1471 | microsomal glutathione | CCTCAACTGAGAAGT |
| | S-transferase 1 (MGST1) gene | GAAGATTCTTCTCTG |
| | , complete cds | CTAGATAGTACGGGG |
| SEQ ID | GI 219521814 NG_009215 Homo sapiens | TTTCTCCATCATGAA |
| NO: 1472 | bone morphogenetic protein 4 (BMP4), | GGTGTAGATATGAGC |
| | RefSeqGene on chromosome 14 | GGTAGTATTGCTGTT |
| | | GCTGGTGGTGTGTGC |
| SEQ ID | GI 1146072968 NG_011674 Homo sapiens | TGACTCACAGGTCCT |
| NO: 1473 | glycine amidinotransferase (GATM), | GACAATTTGACTCTG |
| | RefSeqGene on chromosome 15 | GGGAAATACAGGTCC |
| | | TGACTCACAGATCAT |
| SEQ ID | GI 292658838 NG_016860 Homo sapiens | TGGAAGTTGCAAATT |
| NO: 1474 | pyruvate dehydrogenase (lipoamide | CCTAATTTGGCAGCC |
| | ) beta (PDHB), RefSeqGene on | TGCAGCTGTGATCCA |
| | chromosome 3 | TAGAAGGGAATGTTT |
| SEQ ID | GI 194018566 NG_008183 Homo sapiens | CAAAGGATTAGTCAT |
| NO: 1475 | pancreatic and duodenal homeobox 1 | AGGAAATGGTGGTGG |
| | (PDX1), RefSeqGene on chromosome 13 | GAGGAGGAGGTGGAA |
| | | GGCAAAGAAAAGGAA |
| SEQ ID | GI 181979 M29366 Human epidermal | TACGTAGCTTAGAGG |
| NO: 1476 | growth factor receptor (ERBB3) | CTACAGACTCTGCCT |
| | mRNA, complete cds | TTGATAACCCTGATT |
| | | ACTGGCATAGCAGGC |
| SEQ ID | GI 189491779 NM_006507 Homo sapiens | AGACCATCTCCTCCC |
| NO: 1477 | regenerating family member 1 | TAATGAGTTTGCATC |
| | beta (REGIB), mRNA | GCTGATCTTCAGTAC |
| | | CTTCACCTGTCTCAG |
| SEQ ID | GI 528078360 NM_003955 Homo sapiens | AGGGGATTCTACTCT |
| NO: 1478 | suppressor of cytokine signaling 3 | GTGCCTCCTGACTAT |
| | (SOCS3), mRNA | GTCTGGCTAAGAGAT |
| | | TCGCCTTAAATGCTC |
| SEQ ID | GI 514388123 NG_029053 Homo sapiens | TGAGAACCAGAACAC |
| NO: 1479 | mitogen-activated protein kinase 8 | CGAATGTAGTTTCAG |
| | (MAPK8), RefSeqGene on chromosome 10 | TCCAAAGGCCAGTAG |
| | | GCTCAAGATCTAGGA |
| SEQ ID | GI 342672042 NG_029626 Homo sapiens | GCCTTAGACTGACAG |
| NO: 1480 | synaptosome associated protein 25 | GGACACACTTTGTTC |
| | (SNAP25), RefSeqGene on chromosome 20 | TACTACCACTCATGG |
| | | ACTGTCTCTAGCCAC |
| SEQ ID | GI 1150561155 NG_052944 Homo sapiens | GAACGTGCTACCGGC |
| NO: 1481 | TNF superfamily member 12 (TNFSF12), | ACAGTGAGTGTTTTA |
| | RefSeqGene on chromosome 17 | AGCTGAAGGAATTTG |
| | | AGAAAACCGCAGAAG |
| SEQ ID | GI 95147553 NM_003956 Homo sapiens | GACCTGCATCACTCT |
| NO: 1482 | cholesterol 25-hydroxylase | CACTTTAACTGCAAC |
| | (CH25H), mRNA | TTCGCTCCGTACTTT |
| | | ACACACTGGGACAAA |
| SEQ ID | GI 169790828 NM_002755 Homo sapiens | CCATGGCAATTTTTG |
| NO: 1483 | mitogen-activated protein kinase | AGTTGTTGGATTACA |
| | kinase 1 (MAP2K1), mRNA | TAGTCAACGAGCCTC |
| | | CTCCAAAACTGCCCA |
| SEQ ID | GI 359279909 NM_020663 Homo sapiens | GGGGCAGACCCTTTT |
| NO: 1484 | ras homolog family member J (RHOJ), mRNA | CATAACTCCCTCAAG |
| | | TGTGTGTTACCTTTC |
| | | TTTACCAGCATGGTA |
| SEQ ID | GI 1036032346 AH001409 Homo sapiens | GAGAAGGTCGGACCA |
| NO: 1485 | alcohol dehydrogenase 6 (ADH6) | GCCTTCTGATCTACA |
| | gene, complete cds | GTCGCCTGTGTACCT |
| | | TTGTACTTTCTACAG |
| SEQ ID | GI 219521817 NG_009217 Homo sapiens | TTCCCAGGTACATTC |
| NO: 1486 | cathepsin B (CTSB), RefSeqGene | AAGCTCACAATACCT |
| | on chromosome 8 | TTCCAGCTGCTCCCT |
| | | GATTAGAAAACTGCA |
| SEQ ID | GI 345478700 NG_029900 Homo sapiens | AGGGATATCAGGATG |
| NO: 1487 | p21 | GGGTTTCTCGTGGGA |
| | (RAC1) activated kinase 1 | GGTTTGGTGATCCTG |
| | (PAK1), RefSeqGene on chromosome 11 | ATAGTTAAGTTCCAT |
| SEQ ID | GI 1041818081 NG_051000 Homo sapiens | TGGGATTGTACCTAC |
| NO: 1488 | pentraxin 3 | TGAAATACAGTAGGT |
| | (PTX3), RefSeqGene on chromosome 3 | AACCCTGTGAGGGTC |
| | | CTGGCTTTCATGAGG |
| SEQ ID | GI 109637772 NM_015247 Homo sapiens | AATGCATTAACCAGC |
| NO: 1489 | CYLD lysine 63 deubiquitinase | AAGTGTGGCCAAGGA |
| | (CYLD), transcript variant 1, mRNA | TAATGAAAAAGTGGG |
| | | AAAGGAAGGTCCTCC |
| SEQ ID | GI 256017191 NM_002977 Homo sapiens | TTATGGCTACACGAG |
| NO: 1490 | sodium voltage-gated channel alpha subunit 9 | CTTTGACACTTTCAG |
| | (SCN9A), mRNA | CTGGGCCTTCTTAGC |
| | | CTTGTTTAGGCTAAT |
| SEQ ID | GI 300068920 NG_023233 Homo sapiens | CTCAGATTGAACACT |
| NO: 1491 | bone morphogenetic protein 2 | TACCAGAGTGTCAGG |
| | (BMP2), RefSeqGene on chromosome 20 | GCTTCTGTACCCTGG |
| | | GTTAGAATCAGACCA |
| SEQ ID | GI 383209671 NG_032573 Homo sapiens | TTTCTGTGGTGTTTC |
| NO: 1492 | DnaJ heat shock protein family | CTCCCTTGTATTTTG |
| | (Hsp40) member B6 | TGGTGTTGGTCCCCC |
| | (DNAJB6), RefSeqGene on chromosome | TAACAGGTCTTTGTA |
| SEQ ID | GI 340523111 NG_029470 Homo sapiens | TGCACCAAGAACTCT |
| NO: 1493 | enolase 1 | GACCCATGGTTTCTT |
| | (ENO1), RefSeqGene on chromosome 1 | GCCCCAAAGCAGAAA |
| | | AAAAGTAACTGATGT |
| SEQ ID | GI 347800632 NG_030032 Homo sapiens | ATTTCGCCTATAGCA |
| NO: 1494 | HSPB1 associated protein 1 | TGCTGAACTGGAGTT |
| | (HSPBAP1), RefSeqGene on chromosome 3 | GCAGCAGATGTCAAG |
| | | GTAGAATCCATGGAG |
| SEQ ID | GI 1099522105 NG_052009 Homo sapiens | TCTTACTCATCATCT |
| NO: 1495 | salt inducible kinase 1 | TATGCTGTTACTGTC |
| | (SIK1), RefSeqGene on chromosome 21 | TCCGCAACTGGAACC |
| | | AACGGTCCTCGAGAG |
| SEQ ID | GI 345842418 NG_029946 Homo sapiens | GAAATTTTAGTTGGC |
| NO: 1496 | SMAD family member 2 | TTCTCCTTTGGCAAG |
| | (SMAD2), RefSeqGene on chromosome 18 | CTCAGTCCTTTGTCT |
| | | GTGTCCTGACTTGGC |
| SEQ ID | GI 388596695 NM_001227 Homo sapiens | GAAAATGGAGCCATG |
| NO: 1497 | caspase 7 | ACAAGAACAAAGCCA |
| | (CASP7), transcript variant a, mRNA | CTGACTGAGATGGAG |
| | | TGAGCTGAGACAGAT |
| SEQ ID | GI 306140488 NM_030956 Homo sapiens | TCCATTATGCTTCTC |
| NO: 1498 | toll like receptor 10 | CTCTCTGAGAATCCT |
| | (TLR10), transcript variant 1, mRNA | GACTTACCTCAACAA |
| | | CGGAGACATGGCACA |
| SEQ ID | GI 52854080 AY764389 Homo sapiens | TTCTGAGGCCTTCAC |
| NO: 1499 | baculoviral lAP repeat-containing 3 | TTTGGGTTTTCTGAG |
| | (BIRC3) gene, complete cds | CCCCAACATCTGCTA |
| | | GTGTTGTAAAGAGAA |
| SEQ ID | GI 350606382 NG_030335 Homo sapiens | GTTGTCTGAGTTTGT |
| NO: 1500 | SEL1L ERAD E3 ligase adaptor subunit | CTCACTACCACCCCA |
| | (SELIL), RefSeqGene on chromosome 14 | TACCCGACAGACACC |
| | | ATCTCAACTGTCTTA |
| SEQ ID | GI 325651826 NM_001443 Homo sapiens | TGACCGAACTCAACG |
| NO: 1501 | fatty acid binding protein 1 | GCGACATAATCACCA |
| | (FABP1), mRNA | ATACCATGACATTGG |
| | | GTGACATTGTCTTCA |
| SEQ ID | GI 393537030 NM_000151 Homo sapiens | TGCTTTTGTATGTTT |
| NO: 1502 | glucose-6-phosphatase catalytic subunit | CAATTAGGCTCTGAA |
| | (G6PC), transcript variant 1, mRNA | ATCTTGGGCAAAATG |
| | | ACAAGGGGAGGGCCA |
| SEQ ID | GI 1049017896 NM_002192 Homo sapiens | ATTCAGCAAGGCCCT |
| NO: 1503 | inhibin beta A subunit | CTTTCATTGTACAGG |
| | (INHBA), mRNA | GATCAAATTTCCTCC |
| | | TCTTTTTTGTGCCCC |
| SEQ ID | GI 193211454 NM_020651 Homo sapiens | ATCCTAAGGAGATTG |
| NO: 1504 | pel lino E3 ubiquitin protein ligase 1 | CCACAGTGAGACTTA |
| | (PELI1), mRNA | AGGAAATGTTTGGTT |
| | | GGCAGATGAGCACCA |
| SEQ ID | GI 50234968 AY665769 Homo sapiens | GGGGCAAAGGTAAAA |
| NO: 1505 | chemokine-like factor | CCAAGGAGACCAATT |
| | (CKLF) gene, complete cds | GCAGTAATCTGGGCA |
| | | AGAAATGATGATGGT |
| SEQ ID | GI 238018114 NG_012216 Homo sapiens | TGGCAACCAAGTTTC |
| NO: 1506 | B-cell CLL/lymphoma 10 | CAACATGTGAACTTT |
| | (BCL10), RefSeqGene on chromosome 1 | TGGCGGATACATTCG |
| | | TACCACAGCTAATGG |
| SEQ ID | GI 1025812269 NG_047054 Homo sapiens | AACAGGAAGGCAATC |
| NO: 1507 | glutathione peroxidase 5 | TCTGTTTTTTAAAAA |
| | (GPX5), RefSeqGene on chromosome 6 | GGCCTAGAGGAGGTA |
| | | CCAGTTGGCTGAGCT |
| SEQ ID | GI 336285236 NG_029146 Homo sapiens | AGACGATGTGAAATC |
| NO: 1508 | MCL1, BCL2 family apoptosis regulator | GTTGTCTCGAGTGAT |
| | (MCL1), RefSeqGene on chromosome 1 | GATCCATGTTTTCAG |
| | | CGACGGCGTAACAAA |
| SEQ ID | GI 374349248 NG_032045 Homo sapiens | GGGACCTGAGGACTG |
| NO: 1509 | phospholipase A2 group V | GGAAAGTGAAGTGTT |
| | (PLA2G5), RefSeqGene on chromosome 1 | TTGCTTGAGACCACA |
| | | CAGGTAATAAGGAAG |
| SEQ ID | GI 221316601 NM_001143935 Homo sapiens | CATGAAGGATGTTTG |
| NO: 1510 | carnitine O-octanoyltransferase | GTCACCCCGCCAGAG |
| | (CROT), transcript variant 1, mRNA | CTTCTCAGACAACTG |
| | | ACATATATCCACAAG |
| SEQ ID | GI 27368251 AY196903 Homo sapiens | TGCTTCATCAAAACC |
| NO: 1511 | interleukin 1 receptor antagonist | CCTTTACGTGGGGCT |
| | (ILIRN) gene, complete cds | TAGACACTCCTGTTG |
| | | TCTCTAGTGTCTAGT |
| SEQ ID | GI 237649059 NG_012020 Homo sapiens | ACTGATCCAGCAGTC |
| NO: 1512 | adrenoceptor alpha 2A | AAATGATGAACATGG |
| | (ADRA2A), RefSeqGene on chromosome 10 | TAGGTGGTGAATAAG |
| | | GCAGCTCCTTACAGC |
| SEQ ID | GI 374349244 NG_032042 Homo sapiens | GTCACTGCCTTTCAC |
| NO: 1513 | annexin A5 | AATCCTTCCATCCTT |
| | (ANXA5), RefSeqGene on chromosome 4 | CAATACTTGTGGGTG |
| | | TCACCTTCTCCATGA |
| SEQ ID | GI 1122782504 NG_013302 Homo sapiens | TTCTCTTCCCTTTTG |
| NO: 1514 | catenin beta 1 | GAGGGAGCGTTGTCT |
| | (CTNNB1), RefSeqGene on chromosome 3 | CCTACTTTGTATCTT |
| | | CCAGACATCTGTGGT |
| SEQ ID | GI 189458808 NG_007982 Homo sapiens | CTGCCATTAAATTCC |
| NO: 1515 | cytochrome P450 family 19 | TGTAAAGGAACTTCT |
| | subfamily A member 1 | CACTGAGGGTGCTCA |
| | (CYP19A1), RefSeqGene on chromosome | TTCTGTGTGTGGGGA |
| SEQ ID | GI 335057570 NG_029067 Homo sapiens | TCAGAGACTTCCTAA |
| NO: 1516 | fibroblast growth factor 2 | CAGAGGTACTAGACT |
| | (FGF2), RefSeqGene on chromosome 4 | GGCCTCCTGTTGACA |
| | | CTGAGAGGTTCAGTT |
| SEQ ID | GI 432134281 NG_033037 Homo sapiens | TATAGAGTTCACACA |
| NO: 1517 | ancient ubiquitous protein 1 | TGCTCAGTGTGGTAG |
| | (AUP1); and HtrA serine peptidase 2 | CTGGAGCCTGTTTTC |
| | (HTRA2), RefSeqGene on c [symbol] | CATAGGTTCCTGACT |
| SEQ ID | GI 942429606 NG_008852 Homo sapiens | ACCCGACAACTGTCC |
| NO: 1518 | insulin receptor | AGAGAGAGTGTAAGT |
| | (INSR), RefSeqGene on chromosome 19 | GTAGAAAGGGTTTAA |
| | | GGTGTGTGAGGTGTT |
| SEQ ID | GI 359751452 NG_031884 Homo sapiens | CTTAGGTTGGGCTAA |
| NO: 1519 | mitogen-activated protein | ACATTTTGGATTTTA |
| | kinase kinase kinase 1 | GTCTAAGCCCGGGGG |
| | (MAP3K1), RefSeqGene on chromosome 5 | GAACCACTAGAGCAT |
| SEQ ID | GI 584277161 NG_008225 Homo sapiens | AAGCCAGTAACGTGA |
| NO: 1520 | POU class 1 homeobox 1 | GATGATGTTGTCAGC |
| | (POU1F1), RefSeqGene on chromosome 3 | TAACCACATGCTGCA |
| | | AAGATCAATTTGCCC |
| SEQ ID | GI 746815960 NM_004929 Homo sapiens | AGGGAAGCTGTACCG |
| NO: 1521 | calbindin 1 | AACGGATCTTGCTCT |
| | (CALB1), mRNA | TATTCTCTGTGCTGG |
| | | GGATAACTAGAGTTG |
| SEQ ID | GI 966751447 NM_002581 Homo sapiens | AGCCCTTCATGGGAG |
| NO: 1522 | pappalysin 1 | ACAATTATTGTGATG |
| | (PAPPA), mRNA | CCATCAACAACCGAG |
| | | CCTTTTGCAACTATG |
| SEQ ID | GI 992699 U33447 Human putative | ATGCTGTTCGCCTCC |
| NO: 1523 | G-protein-coupled receptor | TTCTACCTTCTGGAT |
| | (GPR17) gene, complete cds | TTTATCCTGGCTTTA |
| | | GTTGGCAATACCCTG |
| SEQ ID | GI 350276269 NG_030323 Homo sapiens | CCAGTTGGGATCTTG |
| NO: 1524 | sialic acid binding ig like lectin 11 | GTATGAGAACTGACC |
| | (SIGLEC11), RefSeqGene on chromosome 19 | TTGAGTGTATGCCTG |
| | | AGCATTCACTGCATC |
| SEQ ID | GI 959241467 NG_046314 Homo sapiens | GAGCTATGGAAGATG |
| NO: 1525 | signal transducer and | GCACACATTCCCTTT |
| | activator of transcription 2 | CCTACCTCTTCTCCC |
| | (STAT2), RefSeqGene on chromosome 12 | TCTCCCATTACAGAA |
| SEQ ID | GI 229577394 NG_011993 Homo sapiens | AAAAAAAAAAAACAA |
| NO: 1526 | thymocyte selection associated | AGGAAGGTGAGCCCC |
| | high mobility group box | TCCCTTCATGGAGAC |
| | (TOX), RefSeqGene on chromosome [symbol] | TTCTTGGAAGCACCA |
| SEQ ID | GI 112293258 NM_001736 Homo sapiens | TGTTACCTTAGCTAA |
| NO: 1527 | complement C5a receptor 1 | CTAACTCTCCTCCAT |
| | (C5AR1), mRNA | GTTGCCTGTCTTTCC |
| | | CAGACTTGTCCCTCC |
| SEQ ID | GI 22538815 NM_005623 Homo sapiens | CCTGTCTGTAGTGTT |
| NO: 1528 | C-C motif chemokine ligand 8 | GTGGGGTCCTCCCAT |
| | (CCL8), mRNA | GGATCATCAAGGTGA |
| | | AACACTTTGGTATTC |
| SEQ ID | GI 747811824 NM_017594 Homo sapiens | CTGGTTGGACTTGGC |
| NO: 1529 | DIRAS family GTPase 2 | TTCCTGAAAACTCCC |
| | (DIRAS2), mRNA | ACTGATAGTGCCTGT |
| | | TAGATCTCCTGTTTG |
| SEQ ID | GI 169791020 NM_005328 Homo sapiens | ACCTTAGTTCCTCTA |
| NO: 1530 | hyaluronan synthase 2 | GGGGCTGTACAGTAT |
| | (HAS2), mRNA | TGTGGCATCAGATAA |
| | | TGCCACCAAAGGAGA |
| SEQ ID | GI 2304970 U82828 Homo sapiens | TAAGTATCAAGGAAT |
| NO: 1531 | ataxia telangiectasia | TGAGGCCTCCTGCCA |
| | (ATM) gene, complete cds | ACAACTGTTCAAGTG |
| | | TACCATCTTGGAGGC |
| SEQ ID | GI 31559177 AY309922 Homo sapiens | CCTGGTTTGTGAATG |
| NO: 1532 | BH3 interacting domain death agonist | AGCCTAGCTGATTCT |
| | (BID) gene, complete cds | GGCTTTTTCTCCTTT |
| | | CTTGCTCTAGGGGAT |
| SEQ ID | GI 20160210 AY094608 Homo sapiens | CTCTGCCTACATCTG |
| NO: 1533 | cyclin-dependent kinase inhibitor 2C | ACGTCCGGAGTCATT |
| | (CDKN2C) gene, complete cds | AACCAGGAATGTATG |
| | | TGTGTCTGTGTGTCT |
| SEQ ID | GI 662840 L39370 Homo sapiens | TCAAACGGGTCATTG |
| NO: 1534 | heat shock protein 27 | CCATTAATAGAGACC |
| | (HSPB1) gene, complete cds | TCAAACACCGCCTGC |
| | | TAAAAATACCCGACT |
| SEQ ID | GI 82583661 DQ297144 Homo sapiens | GTTCCCAAAGCTCTG |
| NO: 1535 | metallothionein 3 | GAAAATTGTGGAGGG |
| | (growth inhibitory factor | TCAGGAAGAGATCAG |
| | (neurotrophic)) | TTGGACTGAAGACAG |
| | (MT3) gene, complete cds | |
| SEQ ID | GI 1049010853 AH002961 Homo sapiens | TACGCCAGTACCGAA |
| NO: 1536 | N-ras oncogene | TGAAAAAACTCAACA |
| | (NRAS) gene, complete cds | GCAGTGATGATGGGA |
| | | CTCAGGGTTGTATGG |
| SEQ ID | GI 238776831 NG_012261 Homo sapiens | ATGTCCAGTGAAGCA |
| NO: 1537 | aldehyde dehydrogenase 8 family member A1 | GGTAGAACTACCTAG |
| | (ALDH8A1), RefSeqGene on chromosome 6 | AAGTAGGTTCTCAGC |
| | | CACTAGGCCCAGATA |
| SEQ ID | GI 1025608652 NG_047034 Homo sapiens | TTCTAGATCAGGCCC |
| NO: 1538 | autocrine motility factor receptor | CCATTGCCTGTTTCT |
| | (AMFR), RefSeqGene on chromosome 16 | TGAAGAACTTTCTAC |
| | | AGCAGCCAAGAGATG |
| SEQ ID | GI 336088682 NG_029094 Homo sapiens | TCCATAGGCTTCTCC |
| NO: 1539 | apoptotic peptidase activating factor 1 | TAATTCTAGGTGCAG |
| | (APAF1), RefSeqGene on chromosome 12 | CAGTGCATGTGACAC |
| | | TGAAGAACTTCCCTA |
| SEQ ID | GI 283837772 NG_016339 Homo sapiens | ACCTCCATTTTTCTC |
| NO: 1540 | A-Raf proto-oncogene, | CCCGCAACACCATAC |
| | serine/threonine kinase | CTTCCTTCTCCTTTT |
| | (ARAF), RefSeqGene on chromosome X | GCTATGACAATGTCT |
| SEQ ID | GI 343098512 NG_029658 Homo sapiens | TTCCTTCTTACCTCT |
| NO: 1541 | BTB domain and CNC homolog 1 | CTGGCTGTCTCTCCT |
| | (BACH1), RefSeqGene on chromosome 21 | GCCTTCTCTCTCTCT |
| | | CTCAACTGTCTCAAA |
| SEQ ID | GI 238018065 NG_012195 Homo sapiens | GGTTCAGAAGGAGAA |
| NO: 1542 | fatty acid amide hydrolase | AACAAAACAGATGGA |
| | (FAAH), RefSeqGene on chromosome 1 | GGAGGGGCAATGCCT |
| | | CCTAAGTGATTGGTA |
| SEQ ID | GI 1139349906 NG_052802 Homo sapiens | TTTACCCTGTCTCCT |
| NO: 1543 | NLR family pyrin domain containing 4 | TTCTTGAGGCTGATC |
| | (NLRP4), RefSeqGene on chromosome 19 | GATCACAGCCAGGCC |
| | | TCTCCATTCTATTTA |
| SEQ ID | GI 618857519 NG_034119 Homo sapiens | CGAGGTGTCTTGAAG |
| NO: 1544 | nuclear receptor | CAGAAATGTGACTTG |
| | subfamily 2 group F member 1 | GAACTGGACAGACGT |
| | (NR2F1), RefSeqGene on chromosome 5 | TTTCTCAGGAAAGGT |
| SEQ ID | GI 210032246 NG_008862 Homo sapiens | CCTTTGGAACGGCAT |
| NO: 1545 | phosphoglycerate kinase 1 | TAAGCAATCACTAAT |
| | (PGK1), RefSeqGene on chromosome X | GGATGCCCCTGGGAA |
| | | GTCGGTATAACCTTA |
| SEQ ID | GI 256600243 NG_012682 Homo sapiens | TGTTACTGCTGATGG |
| NO: 1546 | versican | ATTCCCAACAGTTTC |
| | (VCAN), RefSeqGene on chromosome 5 | AGTGATGCTGCCTCT |
| | | TCATTCAGAGCAGAA |
| SEQ ID | GI 161377466 NM_003914 Homo sapiens | AGACCCTGTATCTGG |
| NO: 1547 | cyclin A1 | CTGTCAACTTCCTGG |
| | (CCNA1), transcript variant 1, mRNA | ACAGGTTCCTTTCAT |
| | | GTATGTCTGTTCTGA |
| SEQ ID | GI 1135449812 NG_052668 Homo sapiens | TGCAAGAAGATGGTA |
| NO: 1548 | acyl-CoA oxidase 2 | AGATGATCCTTCTCT |
| | (ACOX2), RefSeqGene on chromosome 3 | GGAGAATCTGACCAG |
| | | CCCAAGGAGAAGGAG |
| SEQ ID | GI 255958254 NG_012690 Homo sapiens | AGGCTCTGGTAATGG |
| NO: 1549 | flavin containing monooxygenase 3 | CGACCATTTTCCTGG |
| | (FMO3), RefSeqGene on chromosome 1 | TAACTTAGGGTTATT |
| | | TGTAACTCCAGCAGC |
| SEQ ID | GI 257467500 NG_012841 Homo sapiens | CAGTTTCACCAGTTT |
| NO: 1550 | interleukin 13 receptor subunit alpha 1 | CTCCTCAGAGTGCAG |
| | (IL13RA1), RefSeqGene on chromosome X | CTTTGCCCTGGAAGG |
| | | GATTAAGGATTAGTT |
| SEQ ID | GI 1135449810 NG_052673 Homo sapiens | ACAGCAGTGTATGAA |
| NO: 1551 | 0-6-methylguanine-DNA methyltransferase | ATTGTTCCCATCAGC |
| | (MGMT), RefSeqGene on chromosome 10 | TCCAGCGCGTGAAAG |
| | | ACAATACGAACAGTA |
| SEQ ID | GI 648216408 NG_034146 Homo sapiens | TTCTGGGTTGTGCTA |
| NO: 1552 | phosphoglucomutase 3 | AACAACATAGGGAGG |
| | (PGM3), RefSeqGene on chromosome 6 | AAAGCTGGACCTGGA |
| | | GTCAAAGGAATTGAG |
| SEQ ID | GI 1027250665 NG_047132 Homo sapiens | CTCTCCTCACTTTTT |
| NO: 1553 | peroxisome proliferator-activated receptor | CCTCCAGCATCTCTT |
| | gamma, coactivator-related 1 | TTGGGAAGGCATGCT |
| | (PPRC1), | CCTGTAATCTGACAC |
| | RefSeq | |
| SEQ ID | GI 694879461 NG_031891 Homo sapiens | AACCTCCAAAAATGC |
| NO: 1554 | sodium voltage-gated channel | CAGTGAGAGAAGGCA |
| | alpha subunit 10 | TAGAATAAAGCCAAG |
| | (SCN10A), RefSeqGene on chromosome 3 | AGTGTCAGAGGGCTC |
| SEQ ID | GI 1050193335 NG_027721 Homo sapiens | TGAATGAAACTGCCC |
| NO: 1555 | transforming growth factor beta 2 | CTTGGCCTCTTCCCT |
| | (TGFB2), RefSeqGene on chromosome 1 | ACCATCTCTAAATTG |
| | | CCTTCTTTTCCTTCA |
| SEQ ID | GI 22538799 NM_005408 Homo sapiens | TCACACCCTGAAGAC |
| NO: 1556 | C-C motif chemokine ligand 13 | TTGAACTCTGCTACC |
| | (CCL13), mRNA | CCTACTGAAATCAAG |
| | | CTGGAGTACGTGAAA |
| SEQ ID | GI 325652102 NM_002811 Homo sapiens | CTACGTGGAAGTGAA |
| NO: 1557 | proteasome 26S subunit, non-ATPase 7 | TGGAGACTGATCTCA |
| | (PSMD7), mRNA | AATCTGAACTGCAGC |
| | | TTTCGCTGCTGTGAG |
| SEQ ID | GI 1049010711 AH003036 Homo sapiens | TGTGACCTGGCATGC |
| NO: 1558 | Bruton agammaglobulinemia | CTAGCTACTTTTTCT |
| | (BTK) gene, complete cds | AACTTGCCTAAAGGC |
| | | ACTCAATATGGACGG |
| SEQ ID | GI 28372408 AY228337 Homo sapiens | AGCTCTTCTGTTTGT |
| NO: 1559 | TNF receptor-associated factor 6 | AACACTCTTGCCACA |
| | (TRAF6) gene, complete cds | GCTAACCCCTTAACT |
| | | CTTCCACTTTGGCTC |
| SEQ ID | GI 28372408 AY228337 Homo sapiens | GAGAACACCCAGTCA |
| NO: 1560 | TNF receptor-associated factor 6 | CACATGAGAATGTTG |
| | (TRAF6) gene, complete cds | GCCCAGGCTGTTCAT |
| | | AGTTTGAGCGTTATA |
| SEQ ID | GI 1003701615 NG_008076 Homo sapiens | GTGTGGACCAGCTTT |
| NO: 1561 | growth differentiation factor 5 | GTCATTAACTTGCTG |
| | (GDF5), RefSeqGene on chromosome 20 | TGTGACCTTAGTCAA |
| | | GTCACTCCCCATCTC |
| SEQ ID | GI 974987490 NG_016453 Homo sapiens | CCCTTTCATCATCTT |
| NO: 1562 | transient receptor potential | ATTCTTCAGGGACAG |
| | cation channel subfamily M member 1 | CCATCAGGGTGGGAA |
| | (TRPM1), RefSeqGene or | TTTCAGACCATAGGC |
| SEQ ID | GI 440918690 NM_001272072 Homo sapiens | CGTGGTCTTTGAGCA |
| NO: 1563 | amine oxidase, copper containing 1 | GTTTCTTCACAACAA |
| | (AOC1), transcript variant 1, mRNA | CGAGAACATTGAAAA |
| | | TGAGGACCTGGTGGC |
| SEQ ID | GI 284172478 NM_052946 Homo sapiens | AACGAATGCTTAAAA |
| NO: 1564 | nitric oxide synthase trafficking | CGTACTCCAGCACCT |
| | (NOSTRIN), transcript variant 1, mRNA | CCTCCTTCTCTGATG |
| | | CAAAGAGCCAGAAAG |
| SEQ ID | GI 227499618 NM_003645 Homo sapiens | AAGCGGATTGAAGGC |
| NO: 1565 | solute carrier family 27 member 2 | AGATGATGTCATCTA |
| | (SLC27A2), transcript variant 1, mRNA | TATCACTCTGCCCTT |
| | | TTACCACAGTGCTGC |
| SEQ ID | GI 117606551 EF064750 Homo sapiens | AAGTTGAGGCTAGGA |
| NO: 1566 | CD80 molecule | TTCCTGCACTCAAAG |
| | (CD80) gene, complete cds | AGGTCTGCATATGTT |
| | | AGTTGCGGAAATGCA |
| SEQ ID | GI 27501923 AY204752 Homo sapiens | CATAATGTCCAGAAG |
| NO: 1567 | excision repair cross-complementing | AGAAGGGCTGTTCAC |
| | rodent repair deficiency, | CAGCATGTGCACAAA |
| | complementation group 6 | GTGATCTCAGGAGAG |
| | (ERCC | |
| SEQ ID | GI 163954923 NG_007095 Homo sapiens | GGAGCAGCCTCTCCT |
| NO: 1568 | aldehyde dehydrogenase 3 family member A2 | TGAATTGTTTTGTGA |
| | (ALDH3A2), RefSeqGene on chromosome 17 | TTTGAGTAAAACGGG |
| | | ACTCCCCCTGTTCTA |
| SEQ ID | GI 238018080 NG_012203 Homo sapiens | GTAGTCCTGGTGAAG |
| NO: 1569 | phospholipase A2 group IVA | AGAACAGCCTATCTG |
| | (PLA2G4A), RefSeqGene on chromosome 1 | AAGGCACTGGACTAG |
| | | AAAGGACAGAGCATG |
| SEQ ID | GI 254039651 NG_012413 Homo sapiens | GAAGTTTTTTGCTGC |
| NO: 1570 | vimentin | GTAGACAGTTTTATC |
| | (VIM), RefSeqGene on chromosome 10 | CCCCTCACCCCAAGG |
| | | TCAATTGCACGAATT |
| SEQ ID | GI 161377466 NM_003914 Homo sapiens | CCTCAGCAAGCAATT |
| NO: 1571 | cyclin A1 | AGGGAGAAGTACAAG |
| | (CCNA1), transcript variant 1, mRNA | GCTTCAAAGTACCTG |
| | | TGTGTGTCCCTCATG |
| SEQ ID | GI 735367774 NM_000211 Homo sapiens | TCGACCTGTACTATC |
| NO: 1572 | integrin subunit beta 2 | TGATGGACCTCTCCT |
| | (ITGB2), transcript variant 1, mRNA | ACTCCATGCTTGATG |
| | | ACCTCAGGAATGTCA |
| SEQ ID | GI 393662586 NM_002754 Homo sapiens | CATTCCCCCTAAATC |
| NO: 1573 | mitogen-activated protein kinase 13 | CTCCTGAGCTTTTAG |
| | (MAPK13), transcript variant 1, mRNA | GTAAGAGGCCGATGA |
| | | TTTCACAATCGGCAG |
| SEQ ID | GI 281485607 NM_003226 Homo sapiens | TCCCGGCAAGCGCTT |
| NO: 1574 | trefoil factor 3 | CTGCTGAAAGTTCAT |
| | (TFF3), mRNA | ATCTGGAGCCTGATG |
| | | TCTTAACGAATAAAG |
| SEQ ID | GI 66394595 DQ068066 Homo sapiens | TGCCTAATCACAGTG |
| NO: 1575 | baculoviral lAP repeat-containing 2 | GTTGCTGTCAAGGAG |
| | (BIRC2) gene, complete cds | TATATACACTAGCTG |
| | | GCGAGACTAGTGTGC |
| SEQ ID | GI 117606535 EF064742 Homo sapiens | TTTCACTGAGCACCT |
| NO: 1576 | chemokine | ACTGTGGGCCAGGTC |
| | (C-X-C motif) ligand 16 | ATAGTGTTGATGTCT |
| | (CXCL16) gene, complete cds | TCATCTGATTCCCTA |
| SEQ ID | GI 5821735 AF155233 Homo sapiens | GTACCCTACCTTCTG |
| NO: 1577 | snail zinc finger protein | TCACTTATCAACCCC |
| | (SNAIl) gene, complete cds | TATGAGTGGGGGGTG |
| | | AATAGCCCCATTTTA |
| SEQ ID | GI 30039656 AY273786 Homo sapiens | TGCCACCAACAATTA |
| NO: 1578 | tumor necrosis factor, | TGGAGAAGTCTGCTA |
| | alpha-induced protein 2 | GGTGTCAGCCTCTGT |
| | (TNFAIP2) gene, complete cds | TCTAGTTACCAGGAT |
| SEQ ID | GI 834400736 NG_042182 Homo sapiens | TTCGAAAACAGTGCT |
| NO: 1579 | eomesodermin | CTCCTATTTCTGCCC |
| | (EOMES), RefSeqGene on chromosome 3 | TCTAGAAAGCCCCAA |
| | | AGTGCTAATCACTGC |
| SEQ ID | GI 225543208 NG_011640 Homo sapiens | TATGTTAACATGCAT |
| NO: 1580 | interleukin 6 | GGGAATTCCCTGGGC |
| | (IL6), RefSeqGene on chromosome 7 | ATCTTCTTGTGGTGT |
| | | GGAGTCTGACTTAGC |
| SEQ ID | GI 359338962 NG_031836 Homo sapiens | TGCTACAGGGGAGGT |
| NO: 1581 | Z-DNA binding protein 1 | TATTGTAGTACCGTA |
| | (ZBP1), RefSeqGene on chromosome 20 | TTTGTTCTGAGACAC |
| | | GCTACTTTCCCCCAC |
| SEQ ID | GI 55770831 NM_001802 Homo sapiens | AGCTATACTTCTGGA |
| NO: 1582 | cerebellar degeneration related protein 2 | GAAGTAGGCCTGGAG |
| | (CDR2), mRNA | TTACTACAGTATGGG |
| | | GGAAAAGGGTCGAGT |
| SEQ ID | GI 1135449812 NG_052668 Homo sapiens | TGTGATTTGCAGTTC |
| NO: 1583 | acyl-CoA oxidase 2 | AAGAGTTCACCTGGC |
| | (ACOX2), RefSeqGene on chromosome 3 | AGCTGGGAGAACAAA |
| | | TAAGAGGAACACTGA |
| SEQ ID | GI 696221409 NG_041768 Homo sapiens | TCACTTTGGTACCTT |
| NO: 1584 | CD24 molecule | CACTGCCTTCTCATG |
| | (CD24), RefSeqGene on chromosome 6 | ATTGGCAGTGAAGAA |
| | | CAAAGCTCCACTGAA |
| SEQ ID | GI 507834151 NG_008376 Homo sapiens | 2TCACAACAGTCACCT |
| NO: 1585 | cytochrome P450 family 2 | CCCTTCATATATGAC |
| | subfamily D member 6 | ACCCCAAAACGGAAG |
| | (CYP2D6), RefSeqGene on chromosome 2 | ACAAATCATGGCGTC |
| SEQ ID | GI 893579206 NG_007504 Homo sapiens | ATGCAGGGAGAGCCA |
| NO: 1586 | sonic hedgehog | GCCTGCACTATAAAG |
| | (SHH), RefSeqGene on chromosome 7 | TCCCTGGGGAAGAAA |
| | | AATTAAATAACAGCT |
| SEQ ID | GI 6272556 AF123887 Homo sapiens | CAGTGGATGAATCTC |
| NO: 1587 | EROIL | TGAGTGAGGAAACAC |
| | (EROIL) mRNA, | AGAAGGCTGTTCTTC |
| | | AGTGGACCAAGCATG |
| SEQ ID | GI 57790279 AY865620 Homo sapiens | GTGACAGTGTTCCAA |
| NO: 1588 | signal transducer and activator | GTGAACAAGGCCTGT |
| | of transcription 1, 91 kDa | GAGTGCTAGCCTTGG |
| | (STAT1) gene, complete cds | AACATCTATAGCATC |
| SEQ ID | GI 1025608662 NG_047031 Homo sapiens | TGACCCTTGGTTAGG |
| NO: 1589 | ATP citrate lyase | GCACTTGGTTTGGAG |
| | (ACLY), RefSeqGene on chromosome 17 | CAGGTACTGTACATG |
| | | GAACTTTGTGTAAAA |
| SEQ ID | GI 166795291 NG_007376 Homo sapiens | TGATCTTTGGTCTGC |
| NO: 1590 | amyloid beta precursor protein | CTTCCTTCTCCTTCT |
| | (APP), RefSeqGene on chromosome 21 | CCAAGTCAAACCAGT |
| | | CACTCTTTCCTGAGA |
| SEQ ID | GI 300116194 NG_023256 Homo sapiens | GTTTCTTGCTCTCTC |
| NO: 1591 | linker for activation of T-cells | CCCATCTGTGTGTGT |
| | family member 2 | CTCTGACAGTCTGTG |
| | (LAT2), RefSeqGene on chromosome 7 | GCTCTCTGTATCTTT |
| SEQ ID | GI 327180724 NG_028256 Homo sapiens | GCAGCCTCTTGTGAC |
| NO: 1592 | mitogen-activated protein kinase kinase 3 | CCATGTCAAACATTT |
| | (MAP2K3), RefSeqGene on chromosome 17 | ATTCTGCTTTCTGCA |
| | | TGGTCATGGAGTTCC |
| SEQ ID | GI 340523097 NG_029467 Homo sapiens | TTTGTTCTTCTTGTC |
| NO: 1593 | protein tyrosine kinase 2 | AGGCTTACCCTATGC |
| | (PTK2), RefSeqGene on chromosome 8 | TGCTATAGATGGCTG |
| | | CAACCCGTGTTCAAT |
| SEQ ID | GI 402232739 NM_024295 Homo sapiens | GCCTTCCTTTATCGC |
| NO: 1594 | derlin 1 | TTTCAGATTTGGAGG |
| | (DERL1), transcript variant 1, mRNA | CCAATCACTGCCACC |
| | | TTTTATTTCCCTGTG |
| SEQ ID | GI 1041817991 NM_000569 Homo sapiens | TGGGGTGGAAAAGAA |
| NO: 1595 | Fc fragment of IgG receptor Illa | AGTACAGAACAAACC |
| | (FCGR3A), transcript variant 1, mRNA | CTGTGTCACTGTCCC |
| | | AAGTTGCTAAGTGAA |
| SEQ ID | GI 153792589 NM_001017963 Homo sapiens | CAAGTCTGTGAAGGA |
| NO: 1596 | heat shock protein 90 alpha | TCTGGTCATCTTGCT |
| | family class A member 1 | TTATGAAACTGCGCT |
| | (HSP90AA1), transcript variant 1, n | CCTGTCTTCTGGCTT |
| SEQ ID | GI 38016923 NM_006253 Homo sapiens | TTCTGAAAGGAATGT |
| NO: 1597 | protein kinase AMP-activated | AGAACTTGACAGCAG |
| | non-catalytic subunit beta 1 | CCTTCTGAGTCTGGG |
| | (PRKAB1), mRNA | TCAGGAAGATGTCCT |
| SEQ ID | GI 54112083 AY771996 Homo sapiens | CTTTCATGGTCCCCT |
| NO: 1598 | interleukin 12 receptor, beta 1 | TACCAATCCCCTATT |
| | (IL12RB1) gene, complete cds | CGTGGGCACTTAGAT |
| | | TGCTTCTGGTTTCTC |
| SEQ ID | GI 20303097 AF503166 Homo sapiens | CAACAGAGATGGGAG |
| NO: 1599 | xeroderma pigmentosum, | CTGAGTGCTAGAGTA |
| | complementation group A | GGTGCAGAAGTGGTA |
| | (XPA) gene, complete cds | GGTCAGCTACAAATT |
| SEQ ID | GI 942429606 NG_008852 Homo sapiens | TGACAAATGAAATAG |
| NO: 1600 | insulin receptor | CTCAGATAGGCCTGT |
| | (INSR), RefSeqGene on chromosome 19 | CAGCTAGGGCTCCAA |
| | | CACAGGATTGCAAAG |
| SEQ ID | GI 215820636 NG_009079 Homo sapiens | TAAGATGAGACACAC |
| NO: 1601 | malonyl-CoA decarboxylase | CCAGCAAAGGCTGTC |
| | (MLYCD), RefSeqGene on chromosome 16 | CTCATACAGAACTAC |
| | | CTCCAGGTGTCTTTT |
| SEQ ID | GI 195234781 NG_008319 Homo sapiens | GGTCGGGGAAAAGTG |
| NO: 1602 | pyruvate carboxylase | GAGAGACAAATAGAA |
| | (PC), RefSeqGene on chromosome 11 | AAGAAATAGGGAATT |
| | | GGGACAGGGCACCAT |
| SEQ ID | GI 14594701 AY037115 Homo sapiens | AAGTACCGAGTTCAA |
| NO: 1603 | thymic stromal lymphopoietin | CAACACCGTCTCTTG |
| | (TSLP) mRNA, complete cds | TAGCAATCGGCCACA |
| | | TTGCCTTACTGAAAT |
| SEQ ID | GI 117606571 EF064760 Homo sapiens | CTTCCAGCAGTGCTC |
| NO: 1604 | chemokine | TTGGATCTAGGAGGG |
| | (C-C motif) receptor 3 | AGGAGAAGGAAGGTA |
| | (CCR3) gene, complete cds | CACAGCAATTTAGAG |
| SEQ ID | GI 341865533 NG_029560 Homo sapiens | CAGGAGTCAGGTAGA |
| NO: 1605 | aquaporin 4 | GACGGATGACCTGAT |
| | (AQP4), RefSeqGene on chromosome 18 | TCTAAAACCTGGAGT |
| | | GGTGCATGTGATTGA |
| SEQ ID | GI 224586932 NG_011403 Homo sapiens | ATGACTGTCTGAAGG |
| NO: 1606 | coagulation factor VIII | CAGAAATATCCCAGA |
| | (F8), RefSeqGene on chromosome X | GTACAGGCAGAGACA |
| | | AATGAGGGAGGAAGG |
| SEQ ID | GI 224809244 NG_011449 Homo sapiens | CTCCTTTGAACTTTA |
| NO: 1607 | 3-hydroxy-3-methylglutaryl-CoA reductase | ATGTCCCAGGATTGT |
| | (HMGCR), RefSeqGene on chromosome 5 | AGTGCTGGAGCAGGA |
| | | TTAGACCCAGGCAAC |
| SEQ ID | GI 389565481 NM_002054 Homo sapiens | GTGATGTAAGTTCTT |
| NO: 1608 | glucagon | ATTTGGAAGGCCAAG |
| | (GCG), mRNA | CTGCCAAGGAATTCA |
| | | TTGCTTGGCTGGTGA |
| SEQ ID | GI 1027853651 NM_182828 Homo sapiens | TTTAGGGAGAGGAAG |
| NO: 1609 | growth differentiation factor 7 | GTGTTTGTGCTGATG |
| | (GDF7), mRNA | TTGCAGTTAGAGGCA |
| | | CGAAAATCAGGTAGC |
| SEQ ID | GI 387763637 NM_001098272 Homo sapiens | GAAAGTCTCTTTGCT |
| NO: 1610 | 3-hydroxy-3-methylglutaryl-CoA synthase 1 | CTATTTGCTGACATG |
| | (HMGCS1), transcript variant 1, mRNA | CTTCCTGTTGTGGTC |
| | | TGGCCAATGCCAAAT |
| SEQ ID | GI 117606571 EF064760 Homo sapiens | TGAGAGCCAGACCCC |
| NO: 1611 | chemokine | ATCTCCAGAGACTCT |
| | (C-C motif) receptor 3 | GATTTAATTGTTCAG |
| | (CCR3) gene, complete cds | GGTTATAAGCTGGGG |
| SEQ ID | GI 392055950 JQ867454 Homo sapiens | ACAGAAAAGCCAGAC |
| NO: 1612 | ERBB receptor feedback inhibitor 1 | TCAAAAACAAAAATG |
| | (ERRFIl) gene, complete cds | GATCTGGGTGGCCAC |
| | | GTGAAGCGTAAACAT |
| SEQ ID | GI 32263626 AY325910 Homo sapiens | AAAAAGGGCAGAGTG |
| NO: 1613 | hydroxysteroid | ACATGGCTGAGCTGT |
| | (17-beta) dehydrogenase 2 | TGGAAGCTCCATTGT |
| | (HSD17B2) gene, complete cds | TCTGTAGTGACTTAA |
| SEQ ID | GI 302128095 AB542701 Homo sapiens | TCTCCGAACCTCAGT |
| NO: 1614 | NOD2 gene for nucleotide-binding | TTTTCTATCTGTAAA |
| | oligomerization domain containing 2, | ATGGGGTGACGGGAG |
| | | AGAGGAATGGCAGAA |
| SEQ ID | GI 209447084 NG_008733 Homo sapiens | TGTAAAGTGTTTGCT |
| NO: 1615 | copper chaperone for superoxide dismutase | CTCTGGGAGTAAAGT |
| | (CCS), RefSeqGene on chromosome 11 | CCAACCAGCTCAGGG |
| | | TTGATGATTTGAGGC |
| SEQ ID | GI 19339222 NG_UU/959 Homo sapiens | CTGTGGATTCATGTT |
| NO: 1616 | cytocnrome P450 Tamlly 2/ subfamily | TAGCATGGAGACTGC |
| | a member 1 | CATGAACCTGCATGT |
| | (CYP2/A1), KeTseqsene on chromosome | TTTTTCCTGCTAGGC |
| | [symbol] | |
| SEQ ID | GI 221554534 NG_009447 Homo sapiens | AAGATGTATCTGGCC |
| NO: 1617 | dual oxidase 2 | AGAGGAAGAGGCATT |
| | (DUOX2), RefSeqGene on chromosome 15 | GTTTCTTTTACACAA |
| | | AGGACCTGCAGGGTT |
| SEQ ID | GI 195546896 NG_008330 Homo sapiens | CTGCTGTTTTGTAGC |
| NO: 1618 | integrin subunit alpha 2 | AAGTCAGAAAGCCTG |
| | (ITGA2), RefSeqGene on chromosome 5 | TTCTTTGTCCCACAG |
| | | GGAGATGTTACCTCA |
| SEQ ID | GI 1026191091 NG_047061 Homo sapiens | TCCTGAAAGATCAGG |
| NO: 1619 | marker of proliferation Ki-67 | TCTTTGCGAAGCATA |
| | (MKI67), RefSeqGene on chromosome 10 | TTGGCAAAGAAGGGG |
| | | CAACAAGAATGCAAG |
| SEQ ID | GI 209977115 NM_153692 Homo sapiens | AGCTCTTGACAGTGA |
| NO: 1620 | HtrA serine peptidase 4 | TTCCCTTTCCATGGC |
| | (HTRA4), mRNA | TGTTCTTCGGGGAAA |
| | | AGATAATTTGCTCCT |
| SEQ ID | GI 210147537 NM_025106 Homo sapiens | CGATCAGAATACTGG |
| NO: 1621 | splA/ryanodine receptor domain | AGACGAGACCACGAG |
| | and SOCS box containing 1 | ATTGATGAGTTTGCC |
| | (SPSB1), mRNA | TTGGGAGTCGGTAAG |
| SEQ ID | GI 371875636 NM_182919 Homo sapiens | CAAAAACCCCTGCTC |
| NO: 1622 | toll like receptor adaptor molecule 1 | TGTCAAAGACCAGAC |
| | (TICAM1), mRNA | GCCACTCCAACTTTC |
| | | TGTAGAAGATACCAC |
| SEQ ID | GI 62003164 AY974245 Homo sapiens | ACTGCCCTACCTCCT |
| NO: 1623 | alcohol dehydrogenase 4 | TCAGGCTGACTCCAC |
| | (class II), pi polypeptide | TTATCACTTCCTCTC |
| | (ADH4) gene, complete cds | AGAGATCTACTTCAG |
| SEQ ID | GI 39654020 AY496422 Homo sapiens | GCTTGGAGGTGGAGG |
| NO: 1624 | nuclear factor of kappa light | TGGAGTGGAGAGCAT |
| | polypeptide gene enhancer in | AAATTATATTTCTGC |
| | B-cells inhibitor, alpha | AGGTGAAACCAGAGC |
| | (NFKBIA) gen [symbol] | |
| SEQ ID | GI 1135449812 NG_052668 Homo sapiens | CTTGCCAGGGTTACT |
| NO: 1625 | acyl-CoA oxidase 2 | TCATTTAATCCACAC |
| | (ACOX2), RefSeqGene on chromosome 3 | AGCCCTGGGAGGGAG |
| | | ACTGACATTATTTCC |
| SEQ ID | GI 189409117 NG_007972 Homo sapiens | CATGATCAAGAGCAC |
| NO: 1626 | cytochrome P450 family 2 | CACTCTTAACACCCA |
| | subfamily C member 8 | TGTGCTCCACCCTCA |
| | (CYP2C8), RefSeqGene on chromosome 10 | CAATACACCATCATT |
| SEQ ID | GI 973353146 NG_016274 Homo sapiens | GGGAAGGCTGTTCTG |
| NO: 1627 | hepatocyte growth factor | TGGATGAGTTTAATT |
| | (HGF), RefSeqGene on chromosome 7 | CAGCCCCACAATCAC |
| | | TTCTGTACAGCTACC |
| SEQ ID | GI 343488503 NG_029728 Homo sapiens | GCAGTATTTAGGCAG |
| NO: 1628 | protein kinase cAMP-activated | TTCGGAAGTCTGTAC |
| | catalytic subunit beta | CAGCTTTAACTTTCC |
| | (PRKACB), RefSeqGene on chromosom [symbol] | ACTGGGCCCTCTTTC |
| SEQ ID | GI 975830227 NG_021386 Homo sapiens | CAAATCCCTTGCACT |
| NO: 1629 | TGF-beta activated kinase | TAGGACAACCAAAAC |
| | 1/MAP3K7 binding protein 2 | CAGAAGGCACAATGA |
| | (TAB2), RefSeqGene on chromosome [symbol] | GAAGCCAGCAGTTAC |
| SEQ ID | GI 82880650 NM_004051 Homo sapiens | CCATAGGATGCTTTT |
| NO: 1630 | 3-hydroxybutyrate dehydrogenase 1 | GTACCGTGGACCTTG |
| | (BDHl), transcript variant 1, mRNA | TTCTAGCAGGATCCT |
| | | TTCGGAATTGCACTT |
| SEQ ID | GI 38016940 NM_014365 Homo sapiens | CTTCACAAAGAAAAT |
| NO: 1631 | heat shock protein family B | CCAGCTTCCTGCAGA |
| | (small) member 8 | GGTGGATCCTGTGAC |
| | (HSPB8), mRNA | AGTATTTGCCTCACT |
| SEQ ID | GI 33946335 NM_002166 Homo sapiens | GCAGGCTTCTGAATT |
| NO: 1632 | inhibitor of DNA binding 2, HLH protein | CCCTTCTGAGTTAAT |
| | (ID2), mRNA | GTCAAATGACAGCAA |
| | | AGCACTGTGTGGCTG |
| SEQ ID | GI 163644319 NM_032873 Homo sapiens | TTGGCACATTTTAAT |
| NO: 1633 | ubiquitin associated and SH3 | GCATAAGCTGGGGGT |
| | domain containing B | TTCATTTTCCCAGGC |
| | (UBASH3B), mRNA | TCTCTTCACCATCAC |
| SEQ ID | GI 916259104 NG_042801 Homo sapiens | GAGGTTCATCTCATG |
| NO: 1634 | bromodomain containing 2 | GTTCTGAGGACAGTT |
| | (BRD2), RefSeqGene on chromosome 6 | GAGGAAAGATGGTGG |
| | | GGTCTGTTTGCATTC |
| SEQ ID | GI 584458490 NG_012645 Homo sapiens | TATGCATATGGCTCG |
| NO: 1635 | FK506 binding protein 5 | GCTGGCAGTCTCCCT |
| | (FKBP5), RefSeqGene on chromosome 6 | AAAATTCCCTCGAAT |
| | | GCAACTCTCTTTTTT |
| SEQ ID | GI 374349247 NG_032044 Homo sapiens | ACCCAATGTATTGTA |
| NO: 1636 | glutamic-oxaloacetic transaminase 1 | AAAATGAAATGTCAA |
| | (GOT1), RefSeqGene on chromosome 10 | AGGGAGGCTGCCGCG |
| | | GGTACTCAATGCAAG |
| SEQ ID | GI 270047500 NG_016132 Homo sapiens | ATGGAGAATAGGCTG |
| NO: 1637 | selectin L | TTTGTGTTAGTGAAG |
| | (SELL), RefSeqGene on chromosome 1 | TGTAGGACCTTGGTG |
| | | AGAGCAGTCTCAGGG |
| SEQ ID | GI 40794510 AY497547 Homo sapiens | ACTTCAAGAAGTTCA |
| NO: 1638 | chemokine-like receptor 1 | AGGTGGCCCTCTTCT |
| | (CMKLR1) mRNA, complete cds | CTCGCCTGGTCAATG |
| | | CTCTAAGTGAAGATA |
| SEQ ID | GI 208879434 NM_004895 Homo sapiens | ATCAGGCTGGAGCTG |
| NO: 1639 | NLR family pyrin domain containing 3 | CTGAAATGGATTGAA |
| | (NLRP3), transcript variant 1, mRNA | GTGAAAGCCAAAGCT |
| | | AAAAAGCTGCAGATC |
| SEQ ID | GI 219842351 NM_002522 Homo sapiens | GACAAGTTCCAGCTC |
| NO: 1640 | neuronal pentraxin 1 | ACATTCCCACTGCGG |
| | (NPTX1), mRNA | ACCAACTATATGTAT |
| | | GCCAAGGTGAAGAAG |
| SEQ ID | GI 257743127 NG_012865 Homo sapiens | CCTCACCTGAGAGCT |
| NO: 1641 | amylo-alpha-1, 6-glucosidase, | TGTTAGAAATGCAGA |
| | 4-alpha-glucanotransferase | TTCTTGGACCCTATG |
| | (AGL), RefSeqGene on chromosom [symbol] | CCAAACCTCCCAAAT |
| SEQ ID | GI 1025608649 NG_047040 Homo sapiens | GGCTATTATTGCCCT |
| NO: 1642 | calcium/calmodulin dependent | CTAACAGGAAGACGC |
| | protein kinase 11 alpha | AGAAAGGTGATGTGT |
| | (CAMK2A), RefSeqGene on chromos [symbol] | CAAGTTCAGCGTCAC |
| SEQ ID | GI 1028908925 NG_050621 Homo sapiens | CGAAGAGATCAAAGA |
| NO: 1643 | glutathione peroxidase 4 | GTTCGCCGCGGGCTA |
| | (GPX4), RefSeqGene on chromosome 19 | CAACGTCAAATTCGA |
| | | TATGTTCAGCAAGAT |
| SEQ ID | GI 1140392578 NG_052831 Homo sapiens | AATGCAGAAAAGGCA |
| NO: 1644 | glutamate metabotropic receptor 5 | CAGAAGGAACATCAG |
| | (GRM5), RefSeqGene on chromosome 11 | AGGAAAATGAGGGGG |
| | | AACAGGAACTCTGAG |
| SEQ ID | GI 307133693 NG_027728 Homo sapiens | GCAAGGGAGTCTCCT |
| NO: 1645 | interferon regulatory factor 4 | TTCTCAGCTTCATCG |
| | (IRF4), RefSeqGene on chromosome 6 | CTTCTACTGATTACA |
| | | TGGCTCAGCTCCTAT |
| SEQ ID | GI 325651825 NG_028190 Homo sapiens | CCTAACCACTTCTCT |
| NO: 1646 | notch 4 | GTCTCCGTTGCAATT |
| | (NOTCH4), RefSeqGene on chromosome 6 | TCTCCTTTTTCTCAA |
| | | GCTCCAGCCTAGTGC |
| SEQ ID | GI 1024336758 NG_046923 Homo sapiens | CCTCAAAGCTATGTC |
| NO: 1647 | zinc fingers and homeoboxes 3 | TTCCTGCCATGGAGT |
| | (ZHX3), RefSeqGene on chromosome 20 | GGATTCTCTCTCACT |
| | | GGTGTGTGTACTCTC |
| SEQ ID | GI 68303552 NM_005165 Homo sapiens | CAAATAGCTATGCAG |
| NO: 1648 | aldolase, fructose-bisphosphate C | AGCAGAGATGCCTTC |
| | (ALDOC), mRNA | ACCTGGCACCAACTT |
| | | GTCTTCCTTTCTCTC |
| SEQ ID | GI 82659110 NM_007172 Homo sapiens | CAGACGAGTTGCACA |
| NO: 1649 | nucleoporin 50 | AAATTTTACTGGAGA |
| | (NUP50), transcript variant 2, mRNA | AAAAGGATGCCTGAA |
| | | CACGCAAAGTCGGCT |
| SEQ ID | GI 67515430 DQ088982 Homo sapiens | GCCTTTGATTGGTAC |
| NO: 1650 | glutathione peroxidase 6 | AAGGGCTGTGTAGTG |
| | (olfactory) | TGACTGGCAATGTAG |
| | (GPX6) gene, complete cds | AAGAATGCTCCATCT |
| SEQ ID | GI 269784608 NG_015894 Homo sapiens | AGACCTGTTGGAGGC |
| NO: 1651 | apolipoprotein A5 | TATGAATGCAATCAA |
| | (APOA5), RefSeqGene on chromosome 11 | GGTGACAGACAACTG |
| | | GTGCAATGATGGTAG |
| SEQ ID | GI 224809245 NG_011450 Homo sapiens | TTGCCCTCCACTTTG |
| NO: 1652 | interferon regulatory factor 1 | CTTAACACTCAGTTA |
| | (IRF1), RefSeqGene on chromosome 5 | GGTTCCTTCCTCAGT |
| | | TTCTCCTACCTCCTT |
| SEQ ID | GI 1176461486 NG_030337 Homo sapiens | TAAAAGACCATCCCA |
| NO: 1653 | NLR family CARD domain containing 5 | ACTGTGCTCTACCAC |
| | (NLRC5), RefSeqGene on chromosome 16 | ACTTTGCTCTCCTGG |
| | | CTAAGACTCAGAGAC |
| SEQ ID | GI 205277388 NG_008604 Homo sapiens | TTTCCACAAATAATG |
| NO: 1654 | polycystin 2, transient receptor | CTTCAACTAGTACTT |
| | potential cation channel | GCGTGCCAGTGACTC |
| | (PKD2), RefSeqGene on chromosom [symbol] | CACGTCCCACTCATG |
| SEQ ID | GI 343403846 NG_029699 Homo sapiens | AGAAATAGTCGGAAC |
| NO: 1655 | protein kinase cAMP-activated | TATTGGTCGAAAAGG |
| | catalytic subunit alpha | AAGGCCAGTTTTTGG |
| | (PRKACA), RefSeqGene on chromosom [symbol] | TCTCAAACCCCAGCA |
| SEQ ID | GI 298566320 NM_001005353 Homo sapiens | GTGTGCCCATCTCTT |
| NO: 1656 | adenylate kinase 4 | TGGTAGGGAAGGAGA |
| | (AK4), transcript variant 1, mRNA | AAGGTAAGAATAGCC |
| | | ATCAGTGAGGAAGGA |
| SEQ ID | GI 49066030 AY654286 Homo sapiens | TCACTCAGTTTTTCC |
| NO: 1657 | interferon | TTTACTGGATCTCTG |
| | (alpha, beta and omega) receptor 1 | GGACACTGCTCTTTC |
| | (IFNAR1) gene, complete cds | CTGGTTCTCCTCCTG |
| SEQ ID | GI 54781360 AY789109 Homo sapiens | TCCTGCAAATGTGCA |
| NO: 1658 | interleukin 3 receptor, alpha | AACAGACATGGCTAC |
| | (low affinity) | ATAGAGGCATGCACA |
| | (IL3RA) gene, complete cds | CATATAGACCCATGT |
| SEQ ID | GI 350606343 NG_030328 Homo sapiens | GGCATTTCACCTCTT |
| NO: 1659 | neuropilin 1 | GTCACCTCCAAGTGT |
| | (NRP1), RefSeqGene on chromosome 10 | GTGGTTGAAATAGAG |
| | | GTATCCAACCGTGTT |
| SEQ ID | GI 238776808 NG_012244 Homo sapiens | CCTCACTAGAAACAC |
| NO: 1660 | SMAD family member 6 | TAGATGTGAGGGTTT |
| | (SMAD6), RefSeqGene on chromosome 15 | GGCAAGGGCGTGATA |
| | | GGGACTTACTAGCTG |
| SEQ ID | GI 61676924 NM_012242 Homo sapiens | GACCCAGGCTTGCAA |
| NO: 1661 | dickkopf WNT signaling pathway | AGTGACGGTCATTTT |
| | inhibitor 1 | CTCTTTCTTTCTCCC |
| | (DKK1), mRNA | TCTTGAGTCCTTCTG |
| SEQ ID | GI 1101010097 NG_052015 Homo sapiens | TGTTCCTCAGAAGTG |
| NO: 1662 | adenosine deaminase, RNA specific B1 | TCCAGGTCATGAACA |
| | (ADARB1), RefSeqGene on chromosome 21 | CTGGAAACACCGAGG |
| | | ATTGGTCTAAGCTTA |
| SEQ ID | GI 163954975 NG_007078 Homo sapiens | CACAGAATTGTATCA |
| NO: 1663 | electron transfer flavoprotein | CTGATGAGAGCTGTG |
| | dehydrogenase | GCGACAGAGTGCTCA |
| | (ETFDH), RefSeqGene on chromosome 4 | CCATGTCTCTTTTGC |
| SEQ ID | GI 343168757 NG_029673 Homo sapiens | AATGTCTTCGCACGT |
| NO: 1664 | Fos proto-oncogene, AP-1 | AGGTTTTGCACAGTG |
| | transcription factor subunit | TTTATCTGCTGGTGT |
| | (FOS), RefSeqGene on chromosome 1 [symbol] | CTCAGGGATTTGACA |
| SEQ ID | GI 194018566 NG_008183 Homo sapiens | GAGCAATCTGAGAAG |
| NO: 1665 | pancreatic and duodenal homeobox 1 | CGAATTCGTTTTTCA |
| | (PDX1), RefSeqGene on chromosome 13 | CCAACCGAAAGCAAT |
| | | TGAAGCTGTCTCCCC |
| SEQ ID | GI 38327038 NM_002154 Homo sapiens | ATGAGGAGCCAATGG |
| NO: 1666 | heat shock protein family A | AAACAGATCAGAATG |
| | (Hsp70) member 4 | CAAAGGAGGAAGAGA |
| | (HSPA4), mRNA | AGATGCAAGTGGACC |
| SEQ ID | GI 1036032720 AH002608 Homo sapiens | TGGAGTCACTGCCAA |
| NO: 1667 | amphiregulin | GTCATAGCCATAAAT |
| | (AREG) gene, complete cds | GATGAGTCGGTCCTC |
| | | TTTCCAGTGGATCAT |
| SEQ ID | GI 58802450 AY916056 Homo sapiens | AGGGCTTCAGTGAGG |
| NO: 1668 | flavin containing monooxygenase 2 | AGCTCAGGTCTGGAC |
| | (FMO2) gene, complete cds | TACTTGAACATGAGA |
| | | TGTCTGATGACTCTA |
| SEQ ID | GI 238776813 NG_012250 Homo sapiens | TCTTATCAGTGGAGA |
| NO: 1669 | aldehyde dehydrogenase 2 family | AGGGAGGATGGGTTG |
| | (mitochondrial) | TTGAAAATGCAGCCT |
| | (ALDH2), RefSeqGene on chromosome 12 | GGTTCAACAGTGAAG |
| SEQ ID | GI 292494880 NG_007148 Homo sapiens | GTGTGGTCCCCAAGA |
| NO: 1670 | ATPase phospholipid transporting 8B1 | CCTCCACTAACTTCA |
| | (ATP8B1), RefSeqGene on chromosome 18 | AGTTGTAGTGCAAAG |
| | | GAAGGAAAGCATTCA |
| SEQ ID | GI 1150561157 NG_052945 Homo sapiens | GTAGGTTACTTTCAG |
| NO: 1671 | succinate-CoA ligase GDP- | GGATGGTCTCTGGCC |
| | forming beta subunit | ACAAGTCCCTCAAGC |
| | (SUCLG2), RefSeqGene on chromosome 3 | TAGTTAGAACCTTCC |
| SEQ ID | GI 371877908 NG_031992 Homo sapiens | AATACTGGCTGACCA |
| NO: 1672 | Wnt family member 5A | CCTTGGTGAACTATT |
| | (WNT5A), RefSeqGene on chromosome 3 | GAGCTTGCCTAGGTT |
| | | GACTCATGAGTGAGA |
| SEQ ID | GI 1057867548 NM_020661 Homo sapiens | AGGACCTCCTAATGA |
| NO: 1673 | activation induced cytidine deaminase | GAGTATCTGGGTGAT |
| | (AICDA), transcript variant 1, mRNA | TGTGACCCCAAACCA |
| | | TCTCTCCAAAGCATT |
| SEQ ID | GI 1036029829 AH003667 Homo sapiens | GCAGTCTATGCTGTT |
| NO: 1674 | chromosome 1 leptin receptor | CAGGTGCGCTGTAAG |
| | (LEPR) gene, complete cds | AGGCTAGATGGACTG |
| | | GGATATTGGAGTAAT |
| SEQ ID | GI 336020352 NM_004834 Homo sapiens | CACTCTGACCTCTTG |
| NO: 1675 | mitogen-activated protein | TCAGTTTCCTGTTCC |
| | kinase kinase kinase kinase 4 | TGGTTCCATCTTTTT |
| | (MAP4K4), transcript variant 1, mR [symbol] | GAAAAAGGCCCTCCT |
| SEQ ID | GI 315013554 NG_027996 Homo sapiens | GCACACTGCATACTG |
| NO: 1676 | granzyme A | TTCCTCCAGGGACTG |
| | (GZMA), RefSeqGene on chromosome 5 | AGGTTGACTCTTCTT |
| | | AGAGTGAGACATTCC |
| SEQ ID | GI 388490119 NG_032755 Homo sapiens | ATAAAGTGTTCTCAA |
| NO: 1677 | interferon induced transmembrane protein 3 | GCTGGAAAGATTTAA |
| | (IFITM3), RefSeqGene on chromosome 11 | CAGATTGGCCCCTCG |
| | | GGGACCTGGACATTG |
| SEQ ID | GI 301336145 NG_023427 Homo sapiens | CAAAATCCTTATGGG |
| NO: 1678 | myocyte enhancer factor 2C | AGTATCACTTAGCAC |
| | (MEF2C), RefSeqGene on chromosome 5 | CCAGCCAGGAAGGAA |
| | | TCTCTCATCCCCTCA |
| SEQ ID | GI 1024846751 NG_047008 Homo sapiens | TTCCTCCAAACACCC |
| NO: 1679 | pyruvate dehydrogenase kinase 4 | CAGATCACCCTTAAC |
| | (PDK4), RefSeqGene on chromosome 7 | ATATCCACTGTCTGG |
| | | CACCATCACTACTAC |
| SEQ ID | GI 1140398720 NG_052834 Homo sapiens | CCGATTAAGAAACTG |
| NO: 1680 | TNF receptor superfamily member 9 | GGGGTGAGAGACTAA |
| | (TNFRSF9), RefSeqGene on chromosome 1 | GCAGGCAAGACAGAC |
| | | TGAACTAAGCTGTCA |
| SEQ ID | GI 1126528245 AF058925 Homo sapiens | CTGAAGCTCCTCTTC |
| NO: 1681 | Jak2 kinase mRNA, complete cds | TTGATGACTTTGTCA |
| | | TGTCTTACCTCTTTG |
| | | CTCAGTGGCGGCATG |
| SEQ ID | GI 15128210 AF402002 Homo sapiens | TGTGAAGGCTTTGGG |
| NO: 1682 | interleukin 20 | GGAACTAGACATTCT |
| | (IL20) gene, complete cds | TCTGCAATGGATGGA |
| | | GGAGACAGAATAGGA |
| SEQ ID | GI 685156953 NG_034254 Homo sapiens | AAAGGTGCTTTCTTC |
| NO: 1683 | cyclin D2 | TGGGAGATCTGCTTC |
| | (CCND2), RefSeqGene on chromosome 12 | CTTTAATAACGGTGC |
| | | AAGACCAGTGGTTCG |
| SEQ ID | GI 226509828 NG_011801 Homo sapiens | GACTGAGTGTCTGCT |
| NO: 1684 | carnitine palmitoyltransferase 1A | CACTGTTAGAGGGAC |
| | (CPT1A), RefSeqGene on chromosome 11 | AGGTGCAAAGTGAAT |
| | | TAGTCCATCCAGGAT |
| SEQ ID | GI 388490119 NG_032755 Homo sapiens | TTCAGGTTTTTTTCT |
| NO: 1685 | interferon induced transmembrane protein 3 | ATCTACTGGGAGACT |
| | (IFITM3), RefSeqGene on chromosome 11 | GCCTTTCCTTGGCGC |
| | | TGGTTGCTACCAATT |
| SEQ ID | GI 1142736558 NG_052842 Homo sapiens | CCTTTCCCATTGGCT |
| NO: 1686 | pyruvate dehydrogenase | CAGCTGTTCAGAAGG |
| | phosphatase regulatory subunit | AGAGCTGGACATCTC |
| | (PDPR), RefSeqGene on chromos [symbol] | TTGTTCTTTTCCATT |
| SEQ ID | GI 238018083 NG_012204 Homo sapiens | TGGCACTAACAGCGT |
| NO: 1687 | peroxisome proliferator | TCAAAGAACTTGCGT |
| | activated receptor alpha | GACAAGTGATGACTA |
| | (PPARA), RefSeqGene on chromosome 22 | ATGACACTGAGGGTG |
| SEQ ID | GI 189409110 NM_153000 Homo sapiens | TAGAGAAAAGTGCCT |
| NO: 1688 | APC down-regulated 1 | GGAGGGCTTTTAAGG |
| | (APCDD1), mRNA | AGTCACAGTGCCATC |
| | | ACATGCTCAAACATC |
| SEQ ID | GI 117606557 EF064753 Homo sapiens | ACCGGGTAAGTCCTA |
| NO: 1689 | CD40 ligand | CATGCTCACTGTTGG |
| | (TNF superfamily, member 5, | TTATCTCCCTGGGCT |
| | hyper-IgM syndrome) | CATGGTTTTAACTTA |
| | (CD40LG) gene, complete cds | |
| SEQ ID | GI 19909347 AF495471 Homo sapiens | GATCCTGGAGGATGC |
| NO: 1690 | thrombomodulin | CCAATTAGGGCCTAG |
| | (THBD) gene, complete cds | CCTTAATCAGGTCCT |
| | | CAGAGAATTTCTACC |
| SEQ ID | GI 47419892 AY623660 Homo sapiens | GATATGATTCAGCCT |
| NO: 1691 | TNF receptor-associated factor 2 | TAAAAAGGAAGGCGA |
| | (TRAF2) gene, complete cds | TTCGGACACACACAC |
| | | CCCAGGGATGAACTC |
| SEQ ID | GI 237874189 NG_012169 Homo sapiens | GAATGTGGTCCAGCC |
| NO: 1692 | angiopoietin like 4 | CTTTAGCCTCACTCA |
| | (ANGPTL4), RefSeqGene on chromosome 19 | GTACTTCCTTTGTCC |
| | | CAACTCTATCCCTAC |
| SEQ ID | GI 209977039 NG_008841 Homo sapiens | TGCACTATCTGGCCA |
| NO: 1693 | dopamine receptor D2 | CCCTCAGCCATGAAG |
| | (DRD2), RefSeqGene on chromosome 11 | ACATTATAGGTTGGA |
| | | TGGAAATTTTTCGCA |
| SEQ ID | GI 374253764 NG_032037 Homo sapiens | ATCAGGAGAAACAGA |
| NO: 1694 | SMAD family member 5 | TGCTGTTTAAGTCAG |
| | (SMAD5), RefSeqGene on chromosome 5 | GAAGAAATGAGTGTT |
| | | CGCTCCAAGGCCTGC |
| SEQ ID | GI 48255965 NM_006759 Homo sapiens | ATGTAAAGGGCGGGA |
| NO: 1695 | UDP-glucose pyrophosphorylase 2 | CACTCACTCAATATG |
| | (UGP2), transcript variant 1, mRNA | AAGGCAAACTGAGAC |
| | | TGGTGGAAATTGCTC |
| SEQ ID | GI 209977034 NM_021995 Homo sapiens | CCCTTCTACAGATAC |
| NO: 1696 | urotensin 2 | TGCCAGAGATGCTGG |
| | (UTS2), transcript variant 1, mRNA | GTGCAGAAAGAGGGG |
| | | ATATTCTCAGGAAAG |
| SEQ ID | GI 1049010976 AH002711 Homo sapiens | TTCTAAAGTCGAGCT |
| NO: 1697 | platelet glycoprotein Illa | GGAAGTGCGTGACCT |
| | (ITGB3) gene, complete cds | CCCTGAAGAGTTGTC |
| | | TCTATCCTTCAATGC |
| SEQ ID | GI 156151447 NG_006672 Homo sapiens | TAGAGGTGGCCATGA |
| NO: 1698 | actin, alpha 1, skeletal muscle | GTGCAAAAACCACCA |
| | (ACTA1), RefSeqGene on chromosome 1 | GCTTCAGGAGGAGGA |
| | | AAAAGTAGAAAATCC |
| SEQ ID | GI 298676536 NG_021477 Homo sapiens | AAAACTTCTGCACAC |
| NO: 1699 | glutamate decarboxylase 1 | AGAAGCATTCCAAAA |
| | (GAD1), RefSeqGene on chromosome 2 | AGTCTGTCTCCAGCC |
| | | TGTAGGAGCCAGAAT |
| SEQ ID | GI 342307070 NG_029606 Homo sapiens | AGGGTGAGACTGCGA |
| NO: 1700 | hypoxia inducible factor 1 alpha subunit | CAAAGACTTGAAAAA |
| | (HIF1A), RefSeqGene on chromosome 14 | GGTGGAGAAGCAAGC |
| | | CTTGTGGGTATTTAG |
| SEQ ID | GI 260763977 NG_013058 Homo sapiens | CATCGAGGGTATGAG |
| NO: 1701 | 5-hydroxytryptamine receptor 3A | AACATCCAGAATTTC |
| | (HTR3A), RefSeqGene on chromosome 11 | CCTCCCACCTGTTAT |
| | | ACCTCCTGGAAGCTG |
| SEQ ID | GI 335892866 NG_029106 Homo sapiens | TCCGCCCATAAACTC |
| NO: 1702 | interferon regulatory factor 7 | AGCCCCAACAACTTT |
| | (IRF7), RefSeqGene on chromosome 11 | AGTTTCAAAGTAAGA |
| | | TGGGAACATGGGGAA |
| SEQ ID | GI 325651825 NG_028190 Homo sapiens | GTGAGCACTGATGTG |
| NO: 1703 | notch 4 | GAAATTATTGGAAAA |
| | (NOTCH4), RefSeqGene on chromosome 6 | AGCCATGCCAAGCTC |
| | | ACAGTGGGAAGTGTC |
| SEQ ID | GI 440918690 NM_001272072 Homo sapiens | GTTTCTCATCGAGAT |
| NO: 1704 | amine oxidase, copper containing 1 | GCTGCTGCCCAAGAA |
| | (AOC1), transcript variant 1, mRNA | GTACCATGTGCTGAG |
| | | GTTTCTGGATAAAGG |
| SEQ ID | GI 545746351 NM_006228 Homo sapiens | CAGGAAGTTGGCCAA |
| NO: 1705 | prepronociceptin | TCAGAAGCGGTTCAG |
| | (PNOC), transcript variant 1, mRNA | TGAGTTTATGAGGCA |
| | | ATACTTGGTCCTGAG |
| SEQ ID | GI 542029075 NM_006440 Homo sapiens | CGAAGGTGCCTTGGA |
| NO: 1706 | thioredoxin reductase 2 | ATATGGAATCACAAG |
| | (TXNRD2), transcript variant 1, mRNA | TGATGACATCTTCTG |
| | | GCTGAAGGAATCCCC |
| SEQ ID | GI 198442852 NG_008474 Homo sapiens | ACCCTTGAAGAAAAG |
| NO: 1707 | aryl hydrocarbon receptor interacting | GGGTTTCAATTTTTG |
| | protein like 1 [symbol] | GATGATTAGGCTCAG |
| | (AIPL1), RefSeqGene on chromosome 1 | TAGTCCAGTGGGCCA |
| SEQ ID | GI 254039585 NG_012387 Homo sapiens | AGTGGCTATGGATTG |
| NO: 1708 | aldolase, fructose-bisphosphate B | CAACACATTGGTCTA |
| | (ALDOB), RefSeqGene on chromosome 9 | ATGAGGCCTGGAGAT |
| | | TGGTTCAGAAGCTTT |
| SEQ ID | GI 237820648 NG_012041 Homo sapiens | GAAGTCAGGGAAGGC |
| NO: 1709 | adenosine monophosphate deaminase 3 | CTCTCCAAGGAGATG |
| | (AMPD3), RefSeqGene on chromosome 11 | ACATCTTAGCGGATG |
| | | CTTCAGGGTATAAAG |
| SEQ ID | GI 334085198 NG_029002 Homo sapiens | ATCTGTCTCTCCCGC |
| NO: 1710 | BCL2 like 1 | CTGTAAAATGGAATA |
| | (BCL2L1), RefSeqGene on chromosome 20 | ATGATAGCCAGCTGC |
| | | CATAGCTTGACTCGA |
| SEQ ID | GI 1028908930 NG_050628 Homo sapiens | TTTGGTTGGGTATTC |
| NO: 1711 | nuclear factor kappa B subunit 1 | TGACCCAGCTCAGAG |
| | (NFKB1), RefSeqGene on chromosome 4 | TTTGTGCAAAGCACT |
| | | ATTTCCCAGTACCAG |
| SEQ ID | GI 542133069 NM_004335 Homo sapiens | CTTGAGGGAGAGATC |
| NO: 1712 | bone marrow stromal cell antigen 2 | ACTACATTAAACCAT |
| | (BST2), mRNA | AAGCTTCAGGACGCG |
| | | TCTGCAGAGGTGGAG |
| SEQ ID | GI 219555683 NM_176891 Homo sapiens | GCAGTACCAAAAAGG |
| NO: 1713 | interferon epsilon | ACACACTCTGGCCAT |
| | (IFNE), mRNA | TCTCCATGAGATGCT |
| | | TCAGCAGATCTTCAG |
| SEQ ID | GI 62865623 NM_198057 Homo sapiens | CAGCTTCACCTGACA |
| NO: 1714 | TSC22 domain family member 3 | ACGACTGTTCCTATG |
| | (TSC22D3), transcript variant 1, mRNA | AAGAAGCCACTTGTG |
| | | TTTTAAGCAGAGGCA |
| SEQ ID | GI 67515408 DQ080435 Homo sapiens | GATTCAGAGTGGGCA |
| NO: 1715 | calcitonin/calcitonin- | CATGTTGGGAGACAG |
| | related polypeptide, alpha | TAATGGGTTTGGGTG |
| | (CALCA) gene, complete cds | TGTGTAAATGAGTGT |
| SEQ ID | GI 194018568 NG_008185 Homo sapiens | CGTACTGCATTTGCC |
| NO: 1716 | lactate dehydrogenase A | CCTTGAGCCAGGTGG |
| | (LDHA), RefSeqGene on chromosome 11 | ATGTTTACCGTGTGT |
| | | TATATAACTTCCTGG |
| SEQ ID | GI 401709984 NG_032897 Homo sapiens | CTGCTTCCATTGTCA |
| NO: 1717 | platelet factor 4 | CATCTCCCCTCTCTC |
| | (PF4), RefSeqGene on chromosome 4 | CTCTGACCTTTCTGC |
| | | CTCTAACTCTACCTA |
| SEQ ID | GI 588480539 NG_033255 Homo sapiens | TGGACAGTTGTTCAG |
| NO: 1718 | ras homolog family member H | AGACCACGATCATGT |
| | (RHOH), RefSeqGene on chromosome 4 | GTCTGGTGTCTCACT |
| | | GTGGAATAGTGGTTT |
| SEQ ID | GI 341572552 NG_029517 Homo sapiens | CTGAAGATGGCCCTC |
| NO: 1719 | retinoid X receptor gamma | GCTGTGACATTTAAC |
| | (RXRG), RefSeqGene on chromosome 1 | AGCAACCTCAAAAGT |
| | | GAGAGACCCAAATCT |
| SEQ ID | GI 170014716 NG_007462 Homo sapiens | TTGTAGGTAAGAGCT |
| NO: 1720 | tumor necrosis factor | CTGAGGATGTGTCTT |
| | (TNF), RefSeqGene on chromosome 6 | GGAACTTGGAGGGCT |
| | | AGGATTTGGGGATTG |
| SEQ ID | GI 166158920 NM_013996 Homo sapiens | ATCCTCGTGGCCTTG |
| NO: 1721 | tachykinin precursor 1 | GCAGTCTTTTTTCTT |
| | (TAC1), transcript variant alpha, mRNA | GTCTCCACTCAGCTG |
| | | TTTGCAGAAGAAATA |
| SEQ ID | GI 1025608662 NG_047031 Homo sapiens | AGGCCCCTCCCTTAG |
| NO: 1722 | ATP citrate lyase | AGAATGTGTGTATGT |
| | (ACLY), RefSeqGene on chromosome 17 | GTGTGTTTTCAGGTT |
| | | CCTTGGCATGATTTC |
| SEQ ID | GI 190341054 NG_008010 Homo sapiens | GGTAAAGGAGTGGCA |
| NO: 1723 | aldolase, fructose-bisphosphate A | AGGGAGTCAGAGACA |
| | (ALDOA), RefSeqGene on chromosome 16 | AGAGATCTCTTTGGA |
| | | GAGGCATTCCAGAAG |
| SEQ ID | GI 345525419 NG_029928 Homo sapiens | GCTTGGAAAATGGAG |
| NO: 1724 | CD86 molecule | GCAGCACAGATGGTC |
| | (CD86), RefSeqGene on chromosome 3 | TCCTGCAGAAAGTCT |
| | | GGTGATAAAATGAGA |
| SEQ ID | GI 349585381 NG_030049 Homo sapiens | GGATACCAGCCCCTC |
| NO: 1725 | checkpoint kinase 1 | ATACATTGATAAATT |
| | (CHEK1), RefSeqGene on chromosome 11 | GGTACAAGGGATCAG |
| | | CTTTTCCCAGCCCAC |
| SEQ ID | GI 193794854 NG_008178 Homo sapiens | TACCATTTCCTTCCT |
| NO: 1726 | glycerol kinase | TGTAAGTCTGAAAGT |
| | (GK), RefSeqGene on chromosome X | TCTGGGCTCCAGTCT |
| | | CCCAAGTCCAGTGTT |
| SEQ ID | GI 296923725 NG_012838 Homo sapiens | TGCTTGTCAGGTGGT |
| NO: 1727 | glucose-6-phosphate isomerase | TGGCAGAATTCATTT |
| | (GPI), RefSeqGene on chromosome 19 | CTCTATGGTTGTGGA |
| | | ACTGAAGTCCAGCTT |
| SEQ ID | GI 1031698869 NG_050687 Homo sapiens | CCTGCCTAGCACCGT |
| NO: 1728 | HOP homeobox | GACAGGAATCAGTGC |
| | (HOPX), RefSeqGene on chromosome 4 | TATGATTTTTAGGTG |
| | | GGAAAGCACAGAATC |
| SEQ ID | GI 300244555 NG_023331 Homo sapiens | GCGAACGCTACGCCA |
| NO: 1729 | matrix Gia protein | TGGTTTATGGATACA |
| | (MGP), RefSeqGene on chromosome 12 | ATGCTGCCTATAATC |
| | | GCTACTTCAGGAAGC |
| SEQ ID | GI 388596695 NM_001227 Homo sapiens | TGCTAAGCCAGACCG |
| NO: 1730 | caspase 7 | GTCCTCGTTTGTACC |
| | (CASP7), transcript variant a, mRNA | GTCCCTCTTCAGTAA |
| | | GAAGAAGAAAAATGT |
| SEQ ID | GI 218083972 NM_134431 Homo sapiens | ATGGAGCATCATAGG |
| NO: 1731 | solute carrier organic anion | CCCAGAGAGACATGG |
| | transporter family member 1A2 | CTATGGACTTAGTAC |
| | (SLCO1A2), transcript variant 1, [symbol] | CTTGGCATCACTTTT |
| SEQ ID | GI 62361686 AY987960 Homo sapiens | CCTGGCTGATCCAGG |
| NO: 1732 | alcohol dehydrogenase 5 | GGATCTTGCTAGTAA |
| | (class III), chi polypeptide | CCTCTAACTGAAGCC |
| | (ADH5) gene, complete cds | TCATTCTAGCACATG |
| SEQ ID | GI 29825390 AY242132 Homo sapiens | TTGGATTCCAACCCT |
| NO: 1733 | cannabinoid receptor 2 | ATGAAGGATTACATG |
| | (CNR2) gene, complete cds | ATCCTGAGTGGTCCC |
| | | CAGAAGACAGCTGTT |
| SEQ ID | GI 27501923 AY204752 Homo sapiens | GATTTGGGGAAGTGG |
| NO: 1734 | excision repair cross-complementing | GCCATTTGTAAGGGT |
| | rodent repair deficiency, | TGGTCAGAGAAGACC |
| | complementation group 6 | TTTGATGAACTGACA |
| | (ERCC [symbol] | |
| SEQ ID | GI 994323652 NG_032159 Homo sapiens | TGATGCACCTGTTCC |
| NO: 1735 | CASP2 and RIPK1 domain containing | TCATGGTCTCCAGAC |
| | adaptor with death domain | TTTTCTGTGAAGTAC |
| | (CRADD), RefSeqGene on c [symbol] | AGCATGTAGTGTGGA |
| SEQ ID | GI 1154067877 NG_052975 Homo sapiens | TTTTCTCCTTCCTGG |
| NO: 1736 | C-X-C motif chemokine receptor 2 | GTACAGTGCTATTCT |
| | (CXCR2), RefSeqGene on chromosome 2 | GCCTAGAGGTGAGTT |
| | | TTTGCCTGTCTCACA |
| SEQ ID | GI 269954706 NG_015999 Homo sapiens | TGGCTGGCAATGGTC |
| NO: 1737 | enoyl-CoA hydratase and | TATGTTTACACCTTA |
| | 3-hydroxyacyl CoA dehydrogenase | GCAGAGAGAATTCAC |
| | (EHHADH), RefSeqGene on chr [symbol] | ACTCCTCTGTCCTCC |
| SEQ ID | GI 169790799 NG_007447 Homo sapiens | AGTTCACATCTCCGT |
| NO: 1738 | TGFB induced factor homeobox 1 | TTTTTCCTCCCCCTA |
| | (TGIF1), RefSeqGene on chromosome 18 | ATTCGAAAGAGGCTT |
| | | TCAATTGTCTCCGCC |
| SEQ ID | GI 57863305 NM_052872 Homo sapiens | GTGTAGGAACTTGGG |
| NO: 1739 | interleukin 17F | CTGCATCAATGCTCA |
| | (IL17F), mRNA | AGGAAAGGAAGACAT |
| | | CTCCATGAATTCCGT |
| SEQ ID | GI 82734235 NM_006084 Homo sapiens | TTCTGGGAAGAGAGC |
| NO: 1740 | interferon regulatory factor 9 | CATGGCTCCAGCCAT |
| | (IRF9), mRNA | ACTCCACAGAATCTT |
| | | ATCACAGTGAAGATG |
| SEQ ID | ref\|NG_OO9292.1\| Homo sapiens | CCTCTTGCCTTACTG |
| NO: 1741 | glutaryl-CoAdehydrogenase | AAAGCCCCCTTCTTT |
| | (GCDH), RefSeqGene on | CCCTAAGCCACAATT |
| | | TCCCAGTCTGTAAAG |
| SEQ ID | GI 343168757 NG_029673 Homo sapiens | TCAAGGACTTGAAAG |
| NO: 1742 | Fos proto-oncogene, AP-1 | CATCCATGTGTGGAC |
| | transcription factor subunit | TCAAGTCCTTACCTC |
| | (FOS), RefSeqGene on chromosome 1 [symbol] | TTCCGGAGATGTAGC |
| SEQ ID | GI 401709985 NG_032898 Homo sapiens | CATTCTTGACCCCAT |
| NO: 1743 | plasminogen activator, urokinase receptor | ATCCAACCCCAACTC |
| | (PLAUR), RefSeqGene on chromosome 19 | AACTTCACCTCCAAT |
| | | CCTATGCCCAACTTC |
| SEQ ID | GI 341572553 NG_029518 Homo sapiens | TTCAGTACCTGGACA |
| NO: 1744 | sestrin 1 | GCATTGGAGGATAAT |
| | (SESN1), RefSeqGene on chromosome 6 | GGTCAAGACCATGGG |
| | | CTGTGAAGTTAGTCA |
| SEQ ID | GI 48526521AY644470 Homo sapiens | GATGGGTTCAAATCC |
| NO: 1745 | interferon gamma receptor 2 | TGACTCTGCTTCCAC |
| | (interferon gamma transducer 1) | TTGGCTTCACGTCTT |
| | (IFNGR2) gene, complete cds | GAGCAATCTGCATAT |
| SEQ ID | GI 54781360 AY789109 Homo sapiens | TTTCTTTCAAACCAC |
| NO: 1746 | interleukin 3 receptor, alpha | AGGTCAGAGACAGAA |
| | (low affinity) | CCTCCTTCCAGCTAC |
| | (IL3RA) gene, complete cds | TCAATCCTGGAACGT |
| SEQ ID | GI 457232 L26524 Homo sapiens | GGATCATGGAAAAGT |
| NO: 1747 | PRKDC gene, complete sequence | CATGGGTTAACTGGA |
| | | AGACTTGCTCTGGCA |
| | | GATGGGACAAGGTTT |
| SEQ ID | GI 237820638 NG_012034 Homo sapiens | AAATGCTTCCTGTGA |
| NO: 1748 | ABL proto-oncogene 1, | ATGGAAGATGTGTTT |
| | non-receptor tyrosine kinase | CTGTGCCTCACTGTC |
| | (ABL1), RefSeqGene on chromosome 9 [symbol] | TTCACTGTGCTGGAT |
| SEQ ID | GI 261599023 NG_013217 Homo sapiens | TTTAGCAGCATCCCT |
| NO: 1749 | apoptosis inducing factor | GGCCATTACCCACTG |
| | mitochondria associated 1 | GATATGAGTAGCATC |
| | (AIFM1), RefSeqGene on chromosome [symbol] | CCTACCCAAGTTATT |
| SEQ ID | GI 339639570 NG_029386 Homo sapiens | GGGAATAACCCAGTC |
| NO: 1750 | complement component 4 binding protein beta | TGTGTCCACCTGGTC |
| | (C4BPB), RefSeqGene on chromosome 1 | ATCTGATTTCCTACT |
| | | GCTGCAGGATTCTAA |
| SEQ ID | GI 262331523 NG_013339 Homo sapiens | CTCAGCTTTACTCAA |
| NO: 1751 | catalase | AGCCTCCATGACTTA |
| | (CAT), RefSeqGene on chromosome 11 | GCAGGTTCAGGTTAG |
| | | GCGACCTTCTTAGGT |
| SEQ ID | GI 1028224151 NG_008493 Homo sapiens | AGCGAGGCAAAAGAG |
| NO: 1752 | estrogen receptor 1 | TAGAGATGTTTTAGT |
| | (ESR1), RefSeqGene on chromosome 6 | GAACTAGGCCATGAC |
| | | TTGGGGAGTCTCCAC |
| SEQ ID | GI 225735562 NG_011713 Homo sapiens | TGGTCTTCCAAGTTC |
| NO: 1753 | insulin like growth factor 1 | TCAGTTTCCTGATTT |
| | (IGF1), RefSeqGene on chromosome 12 | GAGTAGCAAGGAGAT |
| | | GGGTCTGGATTCGCT |
| SEQ ID | GI 1142736558 NG_052842 Homo sapiens | AACTCTGAAGAGCAA |
| NO: 1754 | pyruvate dehydrogenase | AAAGGGACAAACACT |
| | phosphatase regulatory subunit | AACTGCTGAGCTGGG |
| | (PDPR), RefSeqGene on chromos [symbol] | CCATTTGATCTCCTT |
| SEQ ID | GI 156523973 NM_001296 Homo sapiens | AGGGCCAGATTCATC |
| NO: 1755 | atypical chemokine receptor 2 | ATTTCAAAGTTACTT |
| | (ACKR2), mRNA | TCTATATGCGGCCGG |
| | | AACAGGGTGGTTGAC |
| SEQ ID | GI 190194362 NM_006426 Homo sapiens | TCGTGAATGACGACC |
| NO: 1756 | dihydropyrimidinase like 4 | AGTCCTTTTACGCTG |
| | (DPYSL4), mRNA | ATGTGCACGTGGAAG |
| | | ATGGCTTGATAAAAC |
| SEQ ID | GI 87046146 DQ142911 Homo sapiens | CTGGAAATCACGTGT |
| NO: 1757 | cogulation factor VII | GTGTAGGTGTGTGGC |
| | (F7) gene, complete cds | ATGTGTATAGGGGTG |
| | | TGTTTATGTGGTGTG |
| SEQ ID | GI 33867482 AY368173 Homo sapiens | CTAACGTGCCTAAGA |
| NO: 1758 | microsomal glutathione S-transferase 1 | TTGGAGGTGACGATA |
| | (MGST1) gene, complete cds | TCTCTGTGATGCTGG |
| | | GGGAGAAATCACAGG |
| SEQ ID | GI 171460912 NG_007496 Homo sapiens | CTCAATGGAAAACGA |
| NO: 1759 | jagged 1 | GCGGAAATAGTGTGG |
| | (JAG1), RefSeqGene on chromosome 20 | GAGGGTAGGAGAGGC |
| | | GATTGATTTGCATTT |
| SEQ ID | GI 1024846744 NG_047021 Homo sapiens | CTGTCCCAGGATAGG |
| NO: 1760 | nudix hydrolase 15 | CAAGTGCTCCTATAC |
| | (NUDT15), RefSeqGene on chromosome 13 | TGACTCTGGCAACAC |
| | | CTTACCAAAAGTCTT |
| SEQ ID | GI 1154491908 NG_052978 Homo sapiens | CCACCACTACAACAA |
| NO: 1761 | pyruvate kinase, muscle | TCAACACAGACTTCT |
| | (PKM), RefSeqGene on chromosome 15 | GTGACCAAATATGTG |
| | | GGGGATTTGCCCTCA |
| SEQ ID | GI 342349297 NG_029616 Homo sapiens | AGAACTTCGTCCAGC |
| NO: 1762 | protein kinase C zeta | TCTACTTTCTGGGGT |
| | (PRKCZ), RefSeqGene on chromosome 1 | CAAGATGAGACCAAG |
| | | CTGTTGGATCTTTGT |
| SEQ ID | GI 294459987 NG_017046 Homo sapiens | AGACAAAGGTGAGAC |
| NO: 1763 | spleen associated tyrosine kinase | TTCCTTTCATTCAAG |
| | (SYK), RefSeqGene on chromosome 9 | GGACATTCACAGAGC |
| | | AAAGTGCGTGGTCAC |
| SEQ ID | GI 480355034 KC485577 Homo sapiens | ATTTGGGCTTACAGC |
| NO: 1764 | bone morphogenenic protein-5 | TCTGTGCAGAAACAG |
| | (BMP5) mRNA, complete cds | GGGATGGACGCAGTA |
| | | TCAACGTAAAATCTG |
| SEQ ID | GI 22122007 AY135686 Homo sapiens | TTTTTGCCGGGAAAG |
| NO: 1765 | growth arrest and | TCGCTACATGGATCA |
| | DNA-damage-inducible, alpha | ATGGGTTCCAGTGAT |
| | (GADD45A) gene, complete cds | TAATCTCCCTGAACG |
| SEQ ID | GI 167887711 EF445033 Homo sapiens | CCACTCCTCCCCCTG |
| NO: 1766 | signal transducing adapter molecule 1 | CAATATTACTGTCTT |
| | (STAM) gene, complete cds, | TAGGTCTTTCCTCTT |
| | alternatively spliced | GGGCTGACATAGAGC |
| SEQ ID | GI 238018096 NG_012212 Homo sapiens | GTAGAGGAGAAGGGG |
| NO: 1767 | C-C motif chemokine ligand 11 | TGTTGCTTGGGCAAA |
| | (CCL11), RefSeqGene on chromosome 17 | ATCCAAGAAGTAGGA |
| | | ATGATCATGCTGCAT |
| SEQ ID | GI 262331528 NG_013342 Homo sapiens | GAAGCTTAGCATAAG |
| NO: 1768 | dihydrolipoamide S-acetyltransferase | GCAGGTGGTACTATG |
| | (DLAT), RefSeqGene on chromosome 11 | GATTTCCTCCTCTTG |
| | | AGGGACTGTGGTGTC |
| SEQ ID | GI 1028224165 NG_047209 Homo sapiens | GAGTCAATCACTACT |
| NO: 1769 | interleukin 33 | CTCAGCAAGCAGCCT |
| | (IL33), RefSeqGene on chromosome 9 | TAACTGGGAATCCTC |
| | | AAAGGGGCATTTGTG |
| SEQ ID | GI 319655745 NG_028087 Homo sapiens | ATAAATGGCTTACGT |
| NO: 1770 | toll like receptor 6 | AACTGCTGCCATCCA |
| | (TLR6), RefSeqGene on chromosome 4 | CACTGTCAAACCATG |
| | | ATGGGCAACCTAAGT |
| SEQ ID | GI 319655745 NG_028087 Homo sapiens | TTCCAGCCCTGAGAA |
| NO: 1771 | toll like receptor 6 | AGTTCTAAGGTGTAC |
| | (TLR6), RefSeqGene on chromosome 4 | AAAACAAAGGCAAGC |
| | | CCCTGAGTGTGAATC |
| SEQ ID | GI 70995318 NM_002506 Homo sapiens | AGCTTTCTATCCTGG |
| NO: 1772 | nerve growth factor | CCACACTGAGGTGCA |
| | (NGF), mRNA | TAGCGTAATGTCCAT |
| | | GTTGTTCTACACTCT |
| SEQ ID | GI 1049010774 AH002843 Homo sapiens | CAGCCTTCTCTGTGT |
| NO: 1773 | interleukin 2 receptor gamma chain | GTATTTCTGGCTGGA |
| | (IL2RG) gene, complete cds | ACGGTGAGATTTGGA |
| | | GAAGCCCAGAAAAAT |
| SEQ ID | GI 224809269 NG_011475 Homo sapiens | CCTGGACCTGTGATG |
| NO: 1774 | toll like receptor 4 | ATTAGGGCTGAATAA |
| | (TLR4), RefSeqGene on chromosome 9 | CGGTGTCTACTTGGG |
| | | TGTAAAAGCCAGGTA |
| SEQ ID | GI 68303552 NM_005165 Homo sapiens | GTGCCTATTGTGGAA |
| NO: 1775 | aldolase, fructose-bisphosphate C | CCTGAAATATTGCCT |
| | (ALDOC), mRNA | GATGGAGACCACGAC |
| | | CTCAAACGTTGTCAG |
| SEQ ID | GI 70995318 NM_002506 Homo sapiens | CGGGGCATTGACTCA |
| NO: 1776 | nerve growth factor | AAGCACTGGAACTCA |
| | (NGF), mRNA | TATTGTACCACGACT |
| | | CACACCTTTGTCAAG |
| SEQ ID | GI 2735714 AF004877 Homo sapiens | GGTATAGGAGCTCCA |
| NO: 1777 | pro-alpha 2(1) collagen | AGGACAAGAAACACG |
| | (COL1A2) gene, complete cds | TCTGGCTAGGAGAAA |
| | | CTATCAATGCTGGCA |
| SEQ ID | GI 32187524 AY324387 Homo sapiens | AGTAGGCAGCAAAGC |
| NO: 1778 | glutathione S-transferase pi | CAAAGAAAATAGCCT |
| | (GSTP1) gene, complete cds | AAGGCACAGCCACTA |
| | | AAAGGAACGTGATCA |
| SEQ ID | GI 40786798 AY518533 Homo sapiens | TAGTGTGTTGAAGGA |
| NO: 1779 | interleukin 17 receptor B | AAATGGGGACAGTGT |
| | (1L17RB) gene, complete cds | TGTCCAAACGTGCTG |
| | | GGCTACTTTGATGAA |
| SEQ ID | GI 3403164 AFO27148 Homo sapiens | GGATGGTGACACTAG |
| NO: 1780 | myogenic determining factor 3 | GGATGGACTGACTTG |
| | (MYOD1) gene, complete cds | AGACAGGTCTTAGAG |
| | | GTAGAATGGACAGCT |
| SEQ ID | GI 1025608652 NG_047034 Homo sapiens | TAAAGGGACTGCAGT |
| NO: 1781 | autocrine motility factor receptor | GTGAAGGTGACCTAC |
| | (AMFR), RefSeqGene on chromosome 16 | TGAATAAAGGTGGCT |
| | | CCCTTACCTGTGTTT |
| SEQ ID | GI 374349244 NG_032042 Homo sapiens | CCTGAAGGAGATTAA |
| NO: 1782 | annexin A5 | GAGTTGGAAGGCTGT |
| | (ANXA5), RefSeqGene on chromosome 4 | CAGCAACCCACATTT |
| | | TCACAGCTTGGAACT |
| SEQ ID | GI 365192584 NG_031951 Homo sapiens | TCCCAGGATAGGTGA |
| NO: 1783 | prostate transmembrane | AAGGGTTAAAATGAG |
| | protein, androgen induced 1 | TAAAGCAGTTGAGCA |
| | (PMEPA1), RefSeqGene on chromoso [symbol] | AACACCAACCCGAGC |
| SEQ ID | GI 229577394 NG_011993 Homo sapiens | AAGTTTGCTTTGATT |
| NO: 1784 | thymocyte selection associated | GGTGGGACCAGTCTT |
| | high mobility group box | GCCCTTGACCTTATC |
| | (TOX), RefSeqGene on chromosome [symbol] | TTAGAGGTTGAAGGC |
| SEQ ID | GI 75677324 NM_014238 Homo sapiens | CTGGGAGTCATGTTT |
| NO: 1785 | kinase suppressor of ras 1 | CTCTTCTCTCTCAGT |
| | (KSR1), mRNA | TCTACTTGGAGCAAG |
| | | AGCTTTCCTGGGCTG |
| SEQ ID | GI 156546893 NM_001101802 Homo sapiens | GAGCGAAAAAGAAGA |
| NO: 1786 | PHD finger protein 21A | ACAACAGCAAATCCG |
| | (PHF21A), transcript variant 1, mRNA | GTCTACAGTGGAGCA |
| | | GTCTTTGAGCCAGAG |
| SEQ ID | GI 14278707 AF365976 Homo sapiens | GATCTATACTGAATC |
| NO: 1787 | interleukin 3 | CCCCAGTCCTCGGGG |
| | (IL3) gene, complete cds | TCTTCTGGACCTTGT |
| | | CCATATCCTTAGGAC |
| SEQ ID | GI 324021724 NG_028154 Homo sapiens | AATGTTAACCCGGTT |
| NO: 1788 | fatty acid binding protein 5 | GCTCGCTGGTGAAAT |
| | (FABP5), RefSeqGene on chromosome 8 | AACTACCGTGACCCT |
| | | CTATTGATACCTGGT |
| SEQ ID | GI 224809429 NG_011538 Homo sapiens | GGTTCAGGCTGTTCT |
| NO: 1789 | potassium voltage-gated channel | TGCCTCTCCTTGGTG |
| | interacting protein 1 | TGTCAGAGTAGAGGA |
| | (KCNIP1), RefSeqGene on chromoso [symbol] | GAAATGACTTTCAGT |
| SEQ ID | GI 344925847 NG_029819 Homo sapiens | ACCTAACAGAGAAAG |
| NO: 1790 | prolactin | CAGATGTGGTCAGTT |
| | (PRL), RefSeqGene on chromosome 6 | CCTGCAACTTTTCTT |
| | | CCGCCTCTCTCTTCT |
| SEQ ID | GI 48772828 AY618665 Homo sapiens | TATGGCTGGTACATC |
| NO: 1791 | VIMP mRNA, complete cds | GTCTTCAGCTGCATC |
| | | CTTCTCTACGTGGTC |
| | | TTTCAGAAGCTTTCC |
| SEQ ID | GI 343488501 NG_029726 Homo sapiens | GAGGTATGGTCATTT |
| NO: 1792 | dynamin 1 | GTGCACAGCCACACA |
| | (DNM1), RefSeqGene on chromosome 9 | GCTCCTACATGACAG |
| | | CTCAGACATTCAATG |
| SEQ ID | GI 908212441 NG_042288 Homo sapiens | TGGGACAGTGGCTTG |
| NO: 1793 | methylsterol monooxygenase 1 | GAAGAGGTAATGGAG |
| | (MSM01), RefSeqGene on chromosome 4 | GCTAGGAAGGTGTTT |
| | | ATCAACAAAGGAAGA |
| SEQ ID | GI 341572553 NG_029518 Homo sapiens | GAGGCTTCACACAGG |
| NO: 1794 | sestrin 1 | GTTTGGCTAACTTGC |
| | (SESN1), RefSeqGene on chromosome 6 | CTTTGTTCGACTGTC |
| | | TTCTGTCATGTAAGG |
| SEQ ID | GI 194353939 NG_008255 Homo sapiens | AGTGAATGGCATCAA |
| NO: 1795 | solute carrier family 19 member 2 | GGTACAAAATGGTGG |
| | (SLC19A2), RefSeqGene on chromosome 1 | CATTGTTACTGACAC |
| | | CCCAGCTTCTAACCA |
| SEQ ID | GI 149408121 NM_024119 Homo sapiens | CCACACGCACAAGGA |
| NO: 1796 | DExH-box helicase 58 | CACCGTCTACAACGT |
| | (DHX58), mRNA | CATCATGAGCCAGTA |
| | | CCTAGAACTTAAACT |
| SEQ ID | GI 1036029697 AH005415 Homo sapiens | ACTTCTTTGCCTTCA |
| NO: 1797 | bradykinin B1 receptor | CTAACAGCTCCCTGA |
| | (BDKRB1) gene, complete cds | ATCCAGTAATTTATG |
| | | TCTTTGTGGGCCGGC |
| SEQ ID | GI 84872755 DQ355026 Homo sapiens | CAAGTGAGAGGCAGA |
| NO: 1798 | caspase 8, apoptosis- | GCTGGGATTTGAATC |
| | related cysteine peptidase | CAGATCTGCCTGATA |
| | (CASP8) gene, complete cds | CTGAAGTCTAGGCTG |
| SEQ ID | GI 3169541 AF043105 Homo sapiens | GGTACTGATGGACTT |
| NO: 1799 | glutathione S-transferase mu 3 | AACCGGAGTTCGGAA |
| | (GSTM3) gene, complete cds | ACCGTCCTGTGTACA |
| | | CATGGGAGTTTAGTG |
| SEQ ID | GI 14278707 AF365976 Homo sapiens | GCAAGACTGGAAATC |
| NO: 1800 | interleukin 3 | TGTCTCCTCACAAGA |
| | (IL3) gene, complete cds | CTGAGCTGCTAGTGG |
| | | AGGTTACCATGTCTG |
| SEQ ID | GI 1025812271 NG_047045 Homo sapiens | CTTCAACAACAGCTT |
| NO: 1801 | activating transcription factor 2 | TTAGTCAGCACTTCC |
| | (ATF2), RefSeqGene on chromosome 2 | CAGCCATGGAGACTC |
| | | CAGGATCTCCTCTTA |
| SEQ ID | GI 340523111 NG_029470 Homo sapiens | CCTTATCCAACCCTG |
| NO: 1802 | enolase 1 | AAGTTCCCTATCTGG |
| | (ENO1), RefSeqGene on chromosome 1 | CTGGAGACCCTCTTT |
| | | CACAAAGATCCCTTT |
| SEQ ID | GI 612339390 NG_034111 Homo sapiens | AACCCCTATGACCCC |
| NO: 1803 | goosecoid homeobox | TCGCCTTCCGTAACT |
| | (GSC), RefSeqGene on chromosome 14 | AAAATAGGCATGGTC |
| | | ATAGTGTCTGTTTCC |
| SEQ ID | GI 210032106 NG_008851 Homo sapiens | AAGCTCAAGGTTTCT |
| NO: 1804 | interleukin 1 beta | GACCTGTATCCCAAG |
| | (ILIB), RefSeqGene on chromosome 2 | TCCCAGAAGCAGAAT |
| | | GGACTAACTCAGAGC |
| SEQ ID | GI 209447063 NG_008722 Homo sapiens | AAAAACCAAGGGACA |
| NO: 1805 | lysyl oxidase | TCAGATTTCTTACCC |
| | (LOX), RefSeqGene on chromosome 5 | AGCCGACCAAGATAT |
| | | TCCTGGGAATGGCAC |
| SEQ ID | GI 874507491 NG_042218 Homo sapiens | CATGAACTGAGGGAC |
| NO: 1806 | surfactant protein D | CTCTATGACATTGGG |
| | (SFTPD), RefSeqGene on chromosome 10 | CAGGGCTTCAAAGGG |
| | | ATCAGATACAGTCTT |
| SEQ ID | GI 392050748 NM_001267774 Homo sapiens | GTAGTAGGCACTCAA |
| NO: 1807 | intraflagellar transport 20 | GAAATGTGTGTTGAA |
| | (IFT20), transcript variant 1, mRNA | TGAACGATGCCTGTG |
| | | ACAAGCAAGCGGACT |
| SEQ ID | GI 1041818013 NM_006065 Homo sapiens | AAGAAGGCCACTTCC |
| NO: 1808 | signal regulatory protein beta 1 | CACGGGTAACAACTG |
| | (SIRPB1), transcript variant 1, mRNA | TTTCAGAACTCACAA |
| | | AGAGAAACAACCTGG |
| SEQ ID | GI 187960074 NG_007666 Homo sapiens | GCAGGGTTTGCCTGA |
| NO: 1809 | HRas proto-oncogene, GTPase | CTGTAAAACGCCTTT |
| | (HRAS), RefSeqGene on chromosome 11 | TTTGTTTTGAGACGA |
| | | AGTAGCGCTCTGTTG |
| SEQ ID | GI 237874160 NG_012149 Homo sapiens | GGGTCCTTGAGATCT |
| NO: 1810 | isocitrate dehydrogenase 3 | CCTTGGCATCCACTC |
| | (NAD(+)) beta | CCATTTCCTATCTGG |
| | (IDH3B), RefSeqGene on chromosome 20 | GACTTCATTATCACC |
| SEQ ID | GI 55770831 NM_001802 Homo sapiens | TCAGAGCATCCATTT |
| NO: 1811 | cerebellar degeneration related protein 2 | GTGAATGGAGTTGAG |
| | (CDR2), mRNA | AAGCTGGTGCCAGAC |
| | | TCTCTGTATGTTCCT |
| SEQ ID | GI 190194395 NG_008003 Homo sapiens | TTCTTCCTCGTTTCT |
| NO: 1812 | acyl-CoA dehydrogenase, | GGTTTTCTCACCATT |
| | short/branched chain | CAGTCGCTCGCATTT |
| | (ACADSB), RefSeqGene on chromosome 10 | CTGGTTCCCTTTTCC |
| SEQ ID | GI 255652920 NG_012644 Homo sapiens | AATGAGGACGGTTAG |
| NO: 1813 | deleted in malignant brain tumors 1 | TCACCCTACAAGTGG |
| | (DMBT1), RefSeqGene on chromosome 10 | CAGATGCTGTGATTG |
| | | GTCCTTGTCATACTC |
| SEQ ID | GI 341823675 NG_029547 Homo sapiens | GTCACTTTGGCATCC |
| NO: 1814 | interleukin 5 receptor subunit alpha | TCTGATGAACTCACA |
| | (IL5RA), RefSeqGene on chromosome 3 | CATGCCTCAGTGCCT |
| | | CAGTGAAAAGAACAG |
| SEQ ID | GI 209977115 NM_153692 Homo sapiens | CATTTAGGAGGGTTT |
| NO: 1815 | HtrA serine peptidase 4 | GCAGGTCCAGAGTAA |
| | (HTRA4), mRNA | AGTCACTGAAGAGTG |
| | | GAAGCGAGGAAGGAA |
| SEQ ID | GI 111161293 NM_005746 Homo sapiens | CTGTTCCTGAGGGCT |
| NO: 1816 | nicotinamide phosphoribosyltransferase | TTGTCATTCCCAGAG |
| | (NAMPT), mRNA | GAAATGTTCTCTTCA |
| | | CGGTGGAAAACACAG |
| SEQ ID | GI 188219598 NM_004688 Homo sapiens | CAACGGGCAAAGAAT |
| NO: 1817 | N-myc and STAT interactor | GGAGGTGGAGAAGTA |
| | (NMI), mRNA | GATGTGGTCAAGTGT |
| | | TCTCTAGGTCAACCT |
| SEQ ID | GI 28932927 AY245248 Homo sapiens | CCAGTCATATGCTGT |
| NO: 1818 | BCL2-interacting killer | CTTTTTGCCCCAGAG |
| | (apoptosis-inducing) | GAGAAATGTCTGAAG |
| | (BIK) gene, complete cds | TAAGACCCCTCTCCA |
| SEQ ID | GI 46361513 AY594694 Homo sapiens | TCTGAGTTTTGACAT |
| NO: 1819 | interferon gamma receptor 1 | CTTGCAGCCTCCTCC |
| | (IFNGR1) gene, complete cds | ATTCTAACAAAATGC |
| | | CAGGGAAAGGCTCTG |
| SEQ ID | GI 77744386 DQ230907 Homo sapiens | AGGAAACAGATCATA |
| NO: 1820 | nudix | GAAGCCTGTGGTTTC |
| | (nucleoside diphosphate linked | ATCCTGTGTTCTCAG |
| | moiety X)-type motif 1 | AGAAGAGGCACCAGG |
| | (NUDT1) gene, complete cds | |
| SEQ ID | GI 32263624 AY325800 Homo sapiens | TTCTCTGAGCCACCA |
| NO: 1821 | stannin | ATTTCTGTATCTGCC |
| | (SNN) gene, complete cds | AAGAGGGAACAGTAA |
| | | TAGCTCAACTGCCCT |
| SEQ ID | GI 383087750 NG_015839 Homo sapiens | CTCCAGAATCGATCC |
| NO: 1822 | ERCC excision repair 1, | CTACTACCTCCTAGT |
| | endonuclease non-catalytic subunit | CCAAGTCACCATTGG |
| | (ERCC1), RefSeqGene on chromo [symbol] | ACTCAACTGGACCAT |
| SEQ ID | GI 226491119 NG_011810 Homo sapiens | TCAGATATGGTGAGG |
| NO: 1823 | 1,4-alpha-glucan branching enzyme 1 | GGAAGAACATGGTGA |
| | (GBE1), RefSeqGene on chromosome 3 | GGGAAAGACCATGAA |
| | | CCCATGGATAGCATC |
| SEQ ID | GI 224809344 NG_011510 Homo sapiens | CGGAAGTCACACACA |
| NO: 1824 | lanosterol synthase | GCATCCTCCCCCACC |
| | (LSS), RefSeqGene on chromosome 21 | ACAGTGGAGTTAAAC |
| | | TGAATTGAAAATCAG |
| SEQ ID | GI 645912997 NG_034139 Homo sapiens | TCCCCAGCACCATTT |
| NO: 1825 | MHC class 1 polypeptide- | TTGAAGAGACTCTCC |
| | related sequence A | TTTGCCAATGTGTGT |
| | (MICA), RefSeqGene on chromosome 6 | TCTTGGTACCTTTGT |
| SEQ ID | GI 209447065 NG_008724 Homo sapiens | TGGCACCACTCACAC |
| NO: 1826 | NLR family apoptosis inhibitory protein | ATGATCACATTATCA |
| | (N AIP), RefSeqGene on chromosome 5 | TGTGACTAGCACCTA |
| | | CTAGAGCCGCACATC |
| SEQ ID | GI 269914179 NG_015979 Homo sapiens | ATGTTAGTCAAGACC |
| NO: 1827 | nitric oxide synthase 1 adaptor protein | TAAGCAGAGGTGGAA |
| | (NOSIAP), RefSeqGene on chromosome 1 | GCACGAGCTTTTCTG |
| | | TCCTACATGCTCCTG |
| SEQ ID | GI 226693341 NG_011851 Homo sapiens | ATAAACACGATTTCC |
| NO: 1828 | synuclein alpha | CTCCATCACTAACTT |
| | (SNCA), RefSeqGene on chromosome 4 | CATCTGCAGGGCCTA |
| | | AGCCTCCTTCACAGT |
| SEQ ID | GI 215598755 NG_009072 Homo sapiens | TACTACACAGGGAAC |
| NO: 1829 | von Willebrand factor | TGGGATTCCTGGCTG |
| | (VWF), RefSeqGene on chromosome 12 | GCAGTAGGAGTTCTT |
| | | GAGTTTTATTGCCAA |
| SEQ ID | GI 206597534 NM_001639 Homo sapiens | TTGGATCAATGGGAC |
| NO: 1830 | amyloid P component, serum | ACCTTTGGTGAAAAA |
| | (APCS), mRNA | GGGTCTGCGACAGGG |
| | | TTACTTTGTAGAAGC |
| SEQ ID | GI 343962631 NM_002037 Homo sapiens | AACCCGCTAAACTTT |
| NO: 1831 | FYN proto-oncogene, Src | CTCATGCAACTAAAA |
| | family tyrosine kinase | TTTCTGGGGGAAATG |
| | (FYN), transcript variant 1, mRNA | AGGGTGGGGGTTGTA |
| SEQ ID | GI 45238846 NM_006142 Homo sapiens | GATTGAGGGAAAGCA |
| NO: 1832 | stratifin | TGTCTGCTGGGTGTG |
| | (SFN), mRNA | ACCATGTTTCCTCTC |
| | | AATAAAGTTCCCCTG |
| SEQ ID | GI 117606559 EF064754 Homo sapiens | CAGGCTCTGAGCCAA |
| NO: 1833 | CD40 molecule, TNF receptor | ACACTGCCTGTTTTC |
| | superfamily member 5 | TTTGTCTGATTTCTC |
| | (CD40) gene, complete cds | ACAACTCCCCCATTA |
| SEQ ID | GI 237649059 NG_012020 Homo sapiens | ACACGAGTATCTGCT |
| NO: 1834 | adrenoceptor alpha 2A | TTAGGAAGATTTCTG |
| | (ADRA2A), RefSeqGene on chromosome 10 | CAACACGGCAGGGTG |
| | | CAATTCAAACTCTGG |
| SEQ ID | GI 238018064 NG_012194 Homo sapiens | GATGTTTGAGCTAGG |
| NO: 1835 | cytochrome b5 reductase 3 | CTCTGAGGAATGAAT |
| | (CYB5R3), RefSeqGene on chromosome 22 | AGGAGTTTGCTGGTT |
| | | GGATGCATGCAGAGC |
| SEQ ID | GI 209447083 NG_008732 Homo sapiens | GGTCCATTTGAATCC |
| NO: 1836 | vascular endothelial growth factor A | AAAGTCCCTGTACTT |
| | (VEGFA), RefSeqGene on chromosome 6 | TCCACTGCCCTACCT |
| | | AGATGTCCCTGTACC |
| SEQ ID | GI 109637772 NM_015247 Homo sapiens | GGACCCCTGTTAGCA |
| NO: 1837 | CYLD lysine 63 deubiquitinase | GAGAGGACAGTCTCC |
| | (CYLD), transcript variant 1, mRNA | GGAATATTCTTTGGA |
| | | GTTGAATTGCTGGAA |
| SEQ ID | GI 372466583 NM_005239 Homo sapiens | GCCAACAGGCTTGGA |
| NO: 1838 | ETS proto-oncogene 2, | TTCCATTTCTCATGA |
| | transcription factor | CTCCGCCAACTGTGA |
| | (ETS2), transcript variant 1, mRNA | ATTGCCTTTGTTAAC |
| SEQ ID | GI 58530885 NM_001570 Homo sapiens | CGAAAGATTCTTCCA |
| NO: 1839 | interleukin 1 receptor associated kinase 2 | GGCAGAGTTGCAGAT |
| | (IRAK2), mRNA | TTGTCTTAGATGCTG |
| | | CCACCCCAATGTCTT |
| SEQ ID | GI 164692857 AK313058 Homo sapiens | TTCTCTACGCCTTTC |
| NO: 1840 | cDNA, FU93535, Homo sapiens | TTGATGAAAACTTCA |
| | opioid receptor, kappa 1 | AGCGGTGTTTCCGGG |
| | (OPRK1), mRNA | ACTTCTGCTTTCCAC |
| SEQ ID | GI 281485607 NM_003226 Homo sapiens | AGGATATTTTGAATC |
| NO: 1841 | trefoil factor 3 | CAGACTGGCACCCAC |
| | (TFF3), mRNA | GTCACAGGAAAGCCT |
| | | AGGAACACTGTAAGT |
| SEQ ID | GI 71648775 DQ143945 Homo sapiens | GTGGCTGTGAAGCTC |
| NO: 1842 | adrenomedullin | AAAGTAGACTGGGAT |
| | (ADM) gene, complete cds | GTGGCCGTGCTTGGT |
| | | AAACTGTAAAATGAT |
| SEQ ID | GI 22788696 AF511593 Homo sapiens | TCCCAGAGTCATCTG |
| NO: 1843 | cyclin D1 | ATTGGACAGGCATGG |
| | (PRAD1: parathyroid adenomatosis 1) | GTGCAAGGAAAATTA |
| | (CCND1) gene, complete cds | GGGTACTCAACCTAA |
| SEQ ID | GI 10799005 AF261108 Homo sapiens | TAGGGAGTGTGATCT |
| NO: 1844 | type XV collagen | TAGAGAGCAGAAGTG |
| | (COL15A1) gene, promoter | AAAAGGAAAGGGAGG |
| | sequence and exon 1, | CGAGGTAGGAGGAAG |
| SEQ ID | GI 170763484 NG_007481 Homo sapiens | ACACCTGAGTCTGTT |
| NO: 1845 | complement C3b/C4b receptor 1 | TGCTCTTCTGTAAAA |
| | (Knops blood group) | TGGGGACACTGGTAT |
| | (CR1), RefSeqGene on chromosome 1 [symbol] | CTCTGCATCGTGGAG |
| SEQ ID | GI 238018064 NG_012194 Homo sapiens | GTTGTCCACCCATTT |
| NO: 1846 | cytochrome b5 reductase 3 | TACAGATGGAGAAAG |
| | (CYB5R3), RefSeqGene on chromosome 22 | CAAGGTGATGTCTCT |
| | | TGCTAAGGCCACCAG |
| SEQ ID | GI 1015224512 NG_023402 Homo sapiens | GGAGACTTTGAGTTT |
| NO: 1847 | Janus kinase 1 | TCTGTGGGTGTAAGT |
| | (JAK1), RefSeqGene on chromosome 1 | TTTGCAAATGGTCCT |
| | | GGTAGCCCTGAAGCT |
| SEQ ID | GI 584458486 NG_012245 Homo sapiens | GTACTGGTGTTTAGG |
| NO: 1848 | N-acetyltransferase 1 | TTGTACTCTGGGGGA |
| | (NAT1), RefSeqGene on chromosome 8 | GATAGCTGCTAGAAG |
| | | TTGGCCCAATTGTGT |
| SEQ ID | GI 238776810 NG_012247 Homo sapiens | AGACTGTCAGTCATC |
| NO: 1849 | solute carrier family 25 member 13 | CTCCTGGAGAAGGCA |
| | (SLC25A13), RefSeqGene on chromosome 7 | TCCTAATTGGCCAGC |
| | | TCTATTTTTGGTAGC |
| SEQ ID | GI 49574519 NM_052972 Homo sapiens | GGGTCCGCTGCAATT |
| NO: 1850 | leucine rich alpha-2-glycoprotein 1 | AGAACGGCTACATCT |
| | (LRG1), mRNA | AGAAGGCAACAAATT |
| | | GCAAGTACTGGGAAA |
| SEQ ID | GI 27501935 AY207429 Homo sapiens | TTACACAATCCTCCT |
| NO: 1851 | interleukin 11 | GGACTCTTGAATACG |
| | (ILH) gene, complete cds | CCCTGTTTTACAGGC |
| | | GAGGGAAACTGAGTC |
| SEQ ID | GI 334085198 NG_029002 Homo sapiens | AACTTTAGCTGCCCA |
| NO: 1852 | BCL2 like 1 | TCAGAATCACCTGGA |
| | (BCL2L1), RefSeqGene on chromosome 20 | AGGTTTGTTAAACCT |
| | | CAGATTGCTGGGCTC |
| SEQ ID | GI 834400736 NG_042182 Homo sapiens | TGAGCCCTCAAAGAC |
| NO: 1853 | eomesodermin | CCAGACTTTTACCTT |
| | (EOMES), RefSeqGene on chromosome 3 | CTCAGAAACGCAATT |
| | | CATTGCAGTGACTGC |
| SEQ ID | GI 319803083 NG_011853 Homo sapiens | TGGAGGCTGTGGCTT |
| NO: 1854 | galactosylceramidase | TTTCCTTTTTCATCC |
| | (GALC), RefSeqGene on chromosome 14 | TCAGGTTGTTTTGGG |
| | | GAGACCTTAAGTCTG |
| SEQ ID | GI 176866166 NG_007524 Homo sapiens | TAGTACATGAGATAG |
| NO: 1855 | KRAS proto-oncogene, GTPase | GCTGTGGGTGGCAAG |
| | (KRAS), RefSeqGene on chromosome 12 | GTTTCTTGAGAAAGT |
| | | GAGGGATGCACAGTT |
| SEQ ID | GI 1154491908 NG_052978 Homo sapiens | ATTGTCGCACGTAAC |
| NO: 1856 | pyruvate kinase, muscle | ACACAGATAATCCCC |
| | (PKM), RefSeqGene on chromosome 15 | TTCCCCCATGTACCT |
| | | ACACAAAGCCATACT |
| SEQ ID | GI 342672042 NG_029626 Homo sapiens | GCAATGAGTCTTGCC |
| NO: 1857 | synaptosome associated protein 25 | TCACTCACAGTAGAG |
| | (SNAP25), RefSeqGene on chromosome 20 | GCACTAGCAGAAGAG |
| | | GCCTTCAGAATACAC |
| SEQ ID | GI 110349787 NM_018489 Homo sapiens | AGAACGATTTCGAGC |
| NO: 1858 | ASH1 like histone lysine | TGAGGAAAAAGGTTG |
| | methyltransferase | GGGAATCAGAACCAA |
| | (ASHIL), mRNA | AGAGCCCCTAAAAGC |
| SEQ ID | GI 20147702 AF493905 Homo sapiens | GGATTCATTGCTTCG |
| NO: 1859 | guanine nucleotide binding | AAGGAGTGACGGCGA |
| | protein alpha il | TCATCTTCTGTGTAG |
| | (GNAIl) mRNA, complete cds | CACTGAGTGACTACG |
| SEQ ID | GI 954169997 NM_002177 Homo sapiens | CCCTTTCTTGTGTCT |
| NO: 1860 | interferon omega 1 | CAAGGACAGAAGAGA |
| | (IFNW1), mRNA | CTTCAGGTTCCCCCA |
| | | GGAGATGGTAAAAGG |
| SEQ ID | GI 54112083 AY771996 Homo sapiens | CAACGGCTGGTTATT |
| NO: 1861 | interleukin 12 receptor, beta 1 | AAGCACTTATGGCAT |
| | (IL12RB1) gene, complete cds | ACCCAGCCCATTCCA |
| | | TCCACTCACATGATT |
| SEQ ID | GI 193290109 NG_008150 Homo sapiens | AGATTGATGTGATTA |
| NO: 1862 | checkpoint kinase 2 | CTTTTCGAACACTTG |
| | (CHEK2), RefSeqGene on chromosome 22 | TCATGCCAGTTGTCT |
| | | TGTGGACCCCGGCAG |
| SEQ ID | GI 340805836 NG_029500 Homo sapiens | ATCTCTGCTGCTTAA |
| NO: 1863 | fatty acid binding protein 6 | TAGCTGGACGACCTT |
| | (FABP6), RefSeqGene on chromosome 5 | AGACAGCCTTCCTAA |
| | | CCTCTCTGAGCTCAA |
| SEQ ID | GI 237858748 NG_012115 Homo sapiens | TAAGCCACTGAGAGC |
| NO: 1864 | phospholipid transfer protein | TGGGGTCTGTCTTTG |
| | (PLTP), RefSeqGene on chromosome 20 | TTAATGCATGCCAAA |
| | | GTGCTTTGGTAAAGT |
| SEQ ID | GI 75709215 NM_181866 Homo sapiens | AATTTTCATGACAAG |
| NO: 1865 | acyl-CoA thioesterase 7 | ATCAGAAAAGGCTGC |
| | (ACOT7), transcript variant hBACHd, mRNA | GTCATCACCATCTCG |
| | | GGACGCATGACCTTC |
| SEQ ID | GI 685844874 NM_002988 Homo sapiens | ACCAACAAAGAGCTC |
| NO: 1866 | C-C motif chemokine ligand 18 | TGCTGCCTCGTCTAT |
| | (CCL18), mRNA | ACCTCCTGGCAGATT |
| | | CCACAAAAGTTCATA |
| SEQ ID | GI 194733730 NM_002993 Homo sapiens | CTACATGGATTCCCT |
| NO: 1867 | C-X-C motif chemokine ligand 6 | ACTTTGAAGAGTGTG |
| | (CXCL6), mRNA | GGGGAAAGCCTACGC |
| | | TTCTCCCTGAAGTTT |
| SEQ ID | GI 345110626 NM_002001 Homo sapiens | AAGAGAACCAGGAAA |
| NO: 1868 | Fc fragment of IgE receptor la | GGCTTCAGACTTCTG |
| | (FCER1A), mRNA | AACCCACATCCTAAG |
| | | CCAAACCCCAAAAAC |
| SEQ ID | GI 378404896 NM_001256796 Homo sapiens | CTTCTAAACTTGGAT |
| NO: 1869 | purinergic receptor P2X 4 | TCCGGATCTGGGATG |
| | (P2RX4), transcript variant 1, mRNA | TGGCGGATTATGTGA |
| | | TACCAGCTCAGGAGG |
| SEQ ID | GI 289666799 NG_016606 Homo sapiens | AGTGCTTTCTAATAA |
| NO: 1870 | glycerol-3-phosphate dehydrogenase 2 | CTCGCATGGTTGTTG |
| | (GPD2), RefSeqGene on chromosome 2 | GACTGAGGTCTCTGT |
| | | TCCTCACCACCTGTT |
| SEQ ID | GI 238018116 NG_012218 Homo sapiens | ATAAACTTTCTAGCG |
| NO: 1871 | hexose-6-phosphate dehydrogenase/ | CATTTTGAGAGAGGG |
| | glucose 1-dehydrogenase | CTTTCTTGGGTGAGG |
| | (H6PD), RefSeqGene on chro [symbol] | GAGCATGGCAAAGTC |
| SEQ ID | GI 340745281 NG_029473 Homo sapiens | AAGATTATTCAGAAG |
| NO: 1872 | heat shock protein family A | GTCGGGGATGGAGCT |
| | (Hsp70) member 8 | AAGCTGCCACCTGGT |
| | (HSPA8), RefSeqGene on chromosome 11 | TAATTAAGGTCCCAA |
| SEQ ID | GI 345525590 NG_029936 Homo sapiens | ACCTCTCCAGGCTGA |
| NO: 1873 | mitogen-activated protein kinase 3 | AACCTCATCATTTTT |
| | (MAPK3), RefSeqGene on chromosome 16 | TTACTGCAGCCATGC |
| | | TGGACAAGTTATGCT |
| SEQ ID | GI 334883167 NG_029059 Homo sapiens | TCCAGGGATCCCTGG |
| NO: 1874 | mitogen-activated protein kinase 9 | TTCCTTTTATGGCGA |
| | (MAPK9), RefSeqGene on chromosome 5 | ATGACATTAGAGAAC |
| | | AAGATCTGGGCTCTG |
| SEQ ID | GI 170014716 NG_007462 Homo sapiens | CGGAACCCAAGCTTA |
| NO: 1875 | tumor necrosis factor | GAACTTTAAGCAACA |
| | (TNF), RefSeqGene on chromosome 6 | AGACCACCACTTCGA |
| | | AACCTGGGATTCAGG |
| SEQ ID | GI 209364548 NM_001912 Homo sapiens | AACATTTGATCACAG |
| NO: 1876 | cathepsin L | TTTAGAGGCACAGTG |
| | (CTSL), transcript variant 1, mRNA | GACCAAGTGGAAGGC |
| | | GATGCACAACAGATT |
| SEQ ID | GI 156104861 NM_181712 Homo sapiens | AGGAAGAGGAAATCA |
| NO: 1877 | KN motif and ankyrin repeat domains 4 | AAGCTAGGGAGCAAA |
| | (KANK4), transcript variant 1, mRNA | GAATTCGAGAGCTGG |
| | | AGTTCACTGTAGCCC |
| SEQ ID | GI 14518294 AF390905 Homo sapiens | TGACTCTACAGACAA |
| NO: 1878 | interleukin 19 | GCTCTATTCTTCCGT |
| | (IL19) gene, complete cds | GGCTGCTCCCACCTG |
| | | AGTTTACTCATCTGA |
| SEQ ID | GI 45505189 AY572796 Homo sapiens | TGAAATCAGGTGGAA |
| NO: 1879 | signal transducer and | ATGACATCTCGCCGC |
| | activator of transcription 3 | TTTCAGCATGCTCTA |
| | (acute-phase response factor) | TTGGTTGGAACAGTT |
| | (STAT3) gene, c [symbol] | |
| SEQ ID | GI 1024336747 NG_046907 Homo sapiens | CAGGGAAATTGAGGG |
| NO: 1880 | acetyl-CoA carboxylase beta | CATCATTGTCATGCG |
| | (ACACB), RefSeqGene on chromosome 12 | CCTTGCCATTCTACC |
| | | ATCTTCTTAAGTGGA |
| SEQ ID | GI 197116377 NG_008385 Homo sapiens | CCTAAAGGCTGCCTG |
| NO: 1881 | cytochrome P450 family | CATTCCTTGGCTCAT |
| | 2 subfamily C member 9 | GAACTCTTCCTTCTT |
| | (CYP2C9), RefSeqGene on chromosome 10 | TCACTCCAAACACTT |
| SEQ ID | GI 297515495 NG_021405 Homo sapiens | AGATGTCCCAATGAC |
| NO: 1882 | MHC class 1 polypeptide- | TAAGAATGTGCGCTG |
| | related sequence B | GGGACAAGCCAATTC |
| | (MICB), RefSeqGene on chromosome 6 | TCTTAGTAGAGGCTT |
| SEQ ID | GI 343887349 NG_029780 Homo sapiens | TGGTGATGAGTTAGT |
| NO: 1883 | X-ray repair cross complementing 5 | ATGGACTACTTGAAC |
| | (XRCC5), RefSeqGene on chromosome 2 | CTCAAAACTGGGCCT |
| | | CTCACCCAAAGCCAA |
| SEQ ID | GI 1047225539 NM_001201 Homo sapiens | TGTCTTTAGTGTCCT |
| NO: 1884 | bone morphogenetic protein 3 | CACTGGTAAAATGAA |
| | (BMP3), mRNA | GAGGCTGGCCATGAG |
| | | CTGGAAGGGTTAAGT |
| SEQ ID | GI 2304970 U82828 Homo sapiens | CCACGTTCCGCAGTC |
| NO: 1885 | ataxia telangiectasia | AAACCAGCCTCTTGA |
| | (ATM) gene, complete cds | TTGCTTCCTCTCTCC |
| | | TTATCCTTCTCATTA |
| SEQ ID | GI 1015635417 AH006868 Homo sapiens | AGATATTTGGCATGA |
| NO: 1886 | CD9 antigen | TCTTCAGTATGATCT |
| | (CD9) gene, complete cds | TGTGCTGTGCTATCC |
| | | GCAGGAACCGCGAGA |
| SEQ ID | GI 34732733 AY388493 Homo sapiens | GGCAGCCAAGAGGAA |
| NO: 1887 | microsomal glutathione S-transferase 3 | TTTGGGAAGTAGTGA |
| | (MGST3) gene, complete cds | AGGAGACCTTTACCC |
| | | CAGGAGTGTTCTATT |
| SEQ ID | ref \| NG_008776.1\| Homo sapiens | AAGAGGGTGATTTGG |
| NO: 1888 | ciliary neurotrophic factor | AAAAGGCTCGCAAAT |
| | (CNTF), RefSeqGene on | CAGCACAAACTTGAA |
| | | AGCCAAGCATGGAAT |
| SEQ ID | GI 359465544 NG_031854 Homo sapiens | AACAAGAGCAGTGAA |
| NO: 1889 | glutamate ionotropic | CCTGGAAGTGACGAA |
| | receptor NMDAtype subunit 2B | CGGGGGATAGATGTT |
| | (GRIN2B), RefSeqGene on chromoso [symbol] | GCTTTTTGTCATTGG |
| SEQ ID | GI 300116187 NG_023252 Homo sapiens | CAAAACTTTGAACAG |
| NO: 1890 | interleukin 4 | CCTCACAGAGCAGAA |
| | (IL4), RefSeqGene on chromosome 5 | GGTGAGTACCTATCT |
| | | GGCACCATCTCTCCA |
| SEQ ID | GI 193211428 NG_008124 Homo sapiens | CTAGCTCACCAGATC |
| NO: 1891 | msh homeobox 2 | GAGATCCTCACTAGA |
| | (MSX2), RefSeqGene on chromosome 5 | ACCCCCAAGTGTAGA |
| | | CACTGGACTTGTGAC |
| SEQ ID | GI 224809269 NG_011475 Homo sapiens | TCATCCATGAAGGTT |
| NO: 1892 | toll like receptor 4 | TCCATAAAAGCCGAA |
| | (TLR4), RefSeqGene on chromosome 9 | AGGTGATTGTTGTGG |
| | | TGTCCCAGCACTTCA |
| SEQ ID | GI 319655745 NG_028087 Homo sapiens | GACTGCAATACCCCA |
| NO: 1893 | toll like receptor 6 | GTCTCTGCCTCTGTT |
| | (TLR6), RefSeqGene on chromosome 4 | TGCAAATGCTCTGCT |
| | | TCTCTGTATCTCTCT |
| SEQ ID | GI 599044742 NM_012234 Homo sapiens | CACACATCCAGTCCT |
| NO: 1894 | RING1 and YYl binding protein | GACATGGAGCTTTTC |
| | (RYBP), mRNA | AGTGTTTGGAGACAT |
| | | TTCTCAATCCCCTGC |
| SEQ ID | GI 270048011 GU143397 Homo sapiens | TCCTTGCCTCCTCAC |
| NO: 1895 | collagen, type III, alpha 1 | CCTTTCTTTCCCCTG |
| | (COL3A1) gene, complete cds | AACTCTCAATCCTTC |
| | | TTCCTTTCTCATTCC |
| SEQ ID | GI 24079970 AF550128 Homo sapiens | GTACTTGTCTGTGTG |
| NO: 1896 | excision repair cross-complementing | TTTGGTGTCCTGAGA |
| | rodent repair deficiency, | GAGGTCAGCCAGGAA |
| | complementation group 5 | GCGACTTGGAAATAT |
| | (ERCC [symbol] | |
| SEQ ID | GI 32165625 AY323826 Homo sapiens | CAAATATCACAGATG |
| NO: 1897 | interleukin 10 receptor, beta | TGGCAGGTCCCTGAA |
| | (IL10RB) gene, complete cds | TCTCTACAGGGTGAG |
| | | TTTCTCACCCTCTGC |
| SEQ ID | GI 300068920 NG_023233 Homo sapiens | CTAACTTACCTCTGT |
| NO: 1898 | bone morphogenetic protein 2 | GACCTGTTTCTGCAA |
| | (BMP2), RefSeqGene on chromosome 20 | CCAGGGGTGTTGCAA |
| | | TGGATGCTTTTGTCT |
| SEQ ID | GI 389565477 NG_032771 Homo sapiens | TGCATAATCCCTGTC |
| NO: 1899 | bone morphogenetic protein 7 | TTCCTATACTCTGTC |
| | (BMP7), RefSeqGene on chromosome 20 | CCGCTGGGATGCCCT |
| | | TAACAATTTATGGGG |
| SEQ ID | GI 227496937 NG_011931 Homo sapiens | GATGTCTGGCTTCTT |
| NO: 1900 | cholinergic receptor | CATCCTATGACTTAG |
| | nicotinic alpha 4 subunit | AGCTTCATCCGTGCT |
| | (CHRNA4), RefSeqGene on chromosome 20 | GTTGTGTGTGTCCGT |
| SEQ ID | GI 300193077 NG_023299 Homo sapiens | GAGGAGCTGGTTGTT |
| NO: 1901 | cAMP responsive element binding protein 1 | AAAGAGCAAACTCAG |
| | (CREB1), RefSeqGene on chromosome 2 | GTAGGGAGGAAACCT |
| | | GGGACATAAGACCAG |
| SEQ ID | GI 678246480 NG_034237 Homo sapiens | CCCCAAACCGCATCT |
| NO: 1902 | cytokine receptor like factor 2 | CTAAACAGTTCCCAC |
| | (CRLF2), RefSeqGene on chromosome X and 7 | AATCACTTCTGCTTT |
| | | GCTTGTACGAAGGAG |
| SEQ ID | GI 315013553 NG_027995 Homo sapiens | TGTCCTGGGTTTGAA |
| NO: 1903 | cystatin A | CCCTTGTTCAATTTG |
| | (CSTA), RefSeqGene on chromosome 3 | TTCTATGGGAAGCTC |
| | | CTCCTCCTCTCTGAG |
| SEQ ID | GI 747813982 NG_007933 Homo sapiens | ACTTGAGTACAGGAA |
| NO: 1904 | cytochrome P450 | CTAGCTTGCAACACA |
| | family 2 subfamily S member 1 | GACTCTAAGCCCACT |
| | (CYP2S1), RefSeqGene on chromosome 19 | CCCATTTCTTCCACC |
| SEQ ID | GI 281182565 NG_016196 Homo sapiens | TCAGGTGTTTATCTC |
| NO: 1905 | endothelin 1 | CTCTGCATTTGTGTA |
| | (EDN1), RefSeqGene on chromosome 6 | ATCTCCCTGAAGGTG |
| | | GATAAGCCAAGGGCA |
| SEQ ID | GI 429535869 NG_033019 Homo sapiens | CATGCATGCTATAGT |
| NO: 1906 | interleukin 5 | TTCATGTGACCCCCA |
| | (IL5), RefSeqGene on chromosome 5 | CCAGCTTTGATGTAT |
| | | TACACCCTGCCCATT |
| SEQ ID | GI 1154067879 NG_052976 Homo sapiens | AGGCTAGGGTAGTTC |
| NO: 1907 | malate dehydrogenase 2 | CAGAGAAGGCAGTGG |
| | (MDH2), RefSeqGene on chromosome 7 | AGGTAACAGAACAGG |
| | | CCTTGTAAAACTTTG |
| SEQ ID | GI 1154067879 NG_052976 Homo sapiens | GAATCAGAAACGGTG |
| NO: 1908 | malate dehydrogenase 2 | ACATTTCTCTTGTGG |
| | (MDH2), RefSeqGene on chromosome 7 | GGTGTTTGTTCTAGG |
| | | CTACCTCGGACCTGA |
| SEQ ID | GI 160358379 NG_007100 Homo sapiens | AAGCTTTGGGTTTGC |
| NO: 1909 | methylmalonyl-CoA mutase | CAACTGTGAAAAGTG |
| | (MUT), RefSeqGene on chromosome 6 | CTCGAATTGCCAGGA |
| | | ACACACAAATCATCA |
| SEQ ID | GI 1024357482 NG_046924 Homo sapiens | CTGCTTCTGTTCCAG |
| NO: 1910 | NLR family pyrin domain containing 5 | GCTTGTTAGCTGGTG |
| | (NLRP5), RefSeqGene on chromosome 19 | GAGAAGCAGTTTATG |
| | | GGAAAAGTTGACGTC |
| SEQ ID | GI 302058260 NG_023447 Homo sapiens | AAGTGCAGTTGTGAG |
| NO: 1911 | resistin | TAGTTAGTTACAACC |
| | (RETN), RefSeqGene on chromosome 19 | AGATACACGCAGGGT |
| | | GCTTACCCCTTCCTG |
| SEQ ID | GI 325588345 HQ995528 Homo sapiens | CAGCCAGAGGACCAG |
| NO: 1912 | testicular nuclear receptor 4 | TCTGCAAGTGAGATA |
| | (NR2C2) mRNA, complete cds | ACTCGGGCATTTGAT |
| | | ACCTTAGCTAAAGCA |
| SEQ ID | GI 164690417 AK312618 Homo sapiens | AATTAGGGACTTATT |
| NO: 1913 | cDNA, FLI93000, Homo sapiens | TCAAATTTGGACATG |
| | RAB5A, member RAS oncogene family | GCTAGTCGAGGCGCA |
| | (RAB5A), mRNA | ACAAGACCCAACGGG |
| SEQ ID | GI 984290537 NM_001319196 Homo sapiens | TAACTTCCAGGAGTT |
| NO: 1914 | S100 calcium binding protein A8 | CCTCATTCTGGTGAT |
| | (S100A8), transcript variant 1, mRNA | AAAGATGGGCGTGGC |
| | | AGCCCACAAAAAAAG |
| SEQ ID | GI 224809429 NG_011538 Homo sapiens | CGGTATGGCTAGTGT |
| NO: 1915 | potassium voltage-gated channel | AGCACTTAACATTGC |
| | interacting protein 1 | TTGGTGCTTGGTGAC |
| | (KCNIP1), RefSeqGene on chromoso [symbol] | ATTCCCAAACGTTCA |
| SEQ ID | GI 874507491 NG_042218 Homo sapiens | GGAAGAAGGGGCCAG |
| NO: 1916 | surfactant protein D | CCTACTTTCCCTATA |
| | (SFTPD), RefSeqGene on chromosome 10 | AGGGATAAAAGTGGG |
| | | TCTCCAATGACCTTC |
| SEQ ID | GI 374253764 NG_032037 Homo sapiens | CACACTGGGACTGTT |
| NO: 1917 | SMAD family member 5 | TAGACATGCATGCCA |
| | (SMAD5), RefSeqGene on chromosome 5 | GTTCATCTGATGTGC |
| | | ACATCTTTGGGATGT |
| SEQ ID | GI 150010588 NM_003641 Homo sapiens | CGCAGAAAACCACAC |
| NO: 1918 | interferon induced | TTCTCAAACCTTCAC |
| | transmembrane protein 1 | TCAACACTTCCTTCC |
| | (IFITM1), mRNA | CCAAAGCCAGAAGAT |
| SEQ ID | GI 169234615 NM_002514 Homo sapiens | TTTGGGTAAACCAGC |
| NO: 1919 | nephroblastoma overexpressed | TCTGAACTTCCAAGC |
| | (NOV), mRNA | TCCAAATCCAAGGAA |
| | | ACATGCAGCTCTTCA |
| SEQ ID | GI 226530635 NG_011844 Homo sapiens | AGAGCCTTCCAACTG |
| NO: 1920 | adenosine deaminase, RNA specific | GAATGAATCCAGTGG |
| | (ADAR), RefSeqGene on chromosome 1 | GTTGTGTTTGTCCAT |
| | | CAAACTTTGACTCCC |
| SEQ ID | GI 363807289 NG_031933 Homo sapiens | TGAAGATTGGAGGAT |
| NO: 1921 | BMP binding endothelial regulator | GCTGAACCACAAACA |
| | (BMPER), RefSeqGene on chromosome 7 | CACAAGGAGAAGAGT |
| | | ACTTCCTCTGCACCA |
| SEQ ID | GI 345525419 NG_029928 Homo sapiens | ACTCCTTTTGGTTTA |
| NO: 1922 | CD86 molecule | TTCTTACCACCTTGC |
| | (CD86), RefSeqGene on chromosome 3 | TTCTGTGTTCCTTGG |
| | | GAATGCTGCTGTGCT |
| SEQ ID | GI 161086962 NG_007114 Homo sapiens | GAGCAAAGCCCTCTA |
| NO: 1923 | insulin | GCCTCATCCTCATAA |
| | (INS), RefSeqGene on chromosome 11 | AAGGTCTCATCATTT |
| | | TCCCTCCAGCCTCTT |
| SEQ ID | GI 237858724 NG_012101 Homo sapiens | CCAGCAACTACTCAC |
| NO: 1924 | matrix metallopeptidase 8 | TCCCTCAAGATGACA |
| | (MMP8), RefSeqGene on chromosome 11 | TCGATGGCATTCAGG |
| | | CCATCTATGGTAAGG |
| SEQ ID | GI 167614492 NM_016583 Homo sapiens | CTGGCTGTGAAGCTG |
| NO: 1925 | BPI fold containing family A member 1 | GACATCACTGCAGAA |
| | (BPIFA1), transcript variant 1, mRNA | ATCTTAGCTGTGAGA |
| | | GATAAGCAGGAGAGG |
| SEQ ID | GI 23110953 NM_001911 Homo sapiens | CGGACCATCCAGAAT |
| NO: 1926 | cathepsin G | GACATCATGTTATTG |
| | (CTSG), mRNA | CAGCTGAGCAGAAGA |
| | | GTCAGACGGAATCGA |
| SEQ ID | GI 50659096 NM_005218 Homo sapiens | GTGGTAACTTTCTCA |
| NO: 1927 | defensin beta 1 | CAGGCCTTGGCCACA |
| | (DEFB1), mRNA | GATCTGATCATTACA |
| | | ATTGCGTCAGCAGTG |
| SEQ ID | GI 118722343 NM_030949 Homo sapiens | CATGAGGAAACTGAG |
| NO: 1928 | protein phosphatase 1 | CCCTCCGCAGAAGAA |
| | regulatory inhibitor subunit 14C | GAGTGTATGATTCTG |
| | (PPP1R14C), mRNA | GAACAGGGTGAAACT |
| SEQ ID | GI 339639570 NG_029386 Homo sapiens | TATTTTCTTCTTCAG |
| NO: 1929 | complement component 4 | TGGGCCACTGTCCTG |
| | binding protein beta | ATCCTGTGCTGGTGA |
| | (C4BPB), RefSeqGene on chromosome 1 | ATGGAGAGTTCAGTT |
| SEQ ID | GI 291219938 NG_016779 Homo sapiens | AGCAAGGAACCCAGA |
| NO: 1930 | interleukin 2 | TTACCTCAAGGTGGC |
| | (IL2), RefSeqGene on chromosome 4 | ACTCTGGGTACTTTT |
| | | TCCTTCTGAGTGATT |
| SEQ ID | GI 584593316 NG_033952 Homo sapiens | GAACTACAACGTGAG |
| NO: 1931 | mitogen-activated protein kinase kinase 4 | CCTTTATTTTGCCTC |
| | (MAP2K4), RefSeqGene on chromosome 17 | GTCTGCAACTCCATC |
| | | CTTAGCCTTTGCGTT |
| SEQ ID | GI 297139734 NG_021330 Homo sapiens | GAGCGATGAGTTCTA |
| NO: 1932 | serum amyloid A1 | TGACTGGTAAAGAAA |
| | (SAA1), RefSeqGene on chromosome 11 | AGGGCCTGCTGGTTT |
| | | CCCTCAGGATCTCTG |
| SEQ ID | GI 298160932 NG_021435 Homo sapiens | CAGAGCACCATGTAC |
| NO: 1933 | vesicle associated membrane protein 7 | TGCCTTTAACTTTCC |
| | (VAMP7), RefSeqGene | TGTCATCCTGTTTGC |
| | on chromosome X and Y | CATGCTGCACTGTAA |
| SEQ ID | GI 16118554 AF398885 Homo sapiens | CCGCAGTAGTATAGA |
| NO: 1934 | ELMOl mRNA, complete cds | GCTGACCAAGATGCT |
| | | ATGTGAGATCTTGAA |
| | | AGTGGGCGAGTTGCC |
| SEQ ID | GI 392996937 NM_001008387 Homo sapiens | CTAGGGGACTACAGA |
| NO: 1935 | regenerating family member 3 gamma | AGGAAAAAGACAAGA |
| | (REG3G), transcript variant 1, mRNA | GGCAGTAGGATATCT |
| | | GTGTGTCCTCCCGCT |
| SEQ ID | GI 669033292 NM_006798 Homo sapiens | CATGGTGTCTCAGGA |
| NO: 1936 | UDP glucuronosyltransferase | GATCTGTGATGGCGT |
| | family 2 member A1 complex locus | TCTTAAAAACCAACA |
| | (UGT2A1), transcript varia [symbol] | GCTGATGGCAAAGCT |
| SEQ ID | GI 193794854 NG_008178 Homo sapiens | AGATGACACACAACT |
| NO: 1937 | glycerol kinase | CCTGCCCAAGTGATT |
| | (GK), RefSeqGene on chromosome X | TGTCTTCCGTTTGCA |
| | | CTACCTGGTCTAAGA |
| SEQ ID | GI 171460912 NG_007496 Homo sapiens | CAAAGAATTGAACCC |
| NO: 1938 | jagged 1 | CGATCCGGTTCACAG |
| | (JAG1), RefSeqGene on chromosome 20 | AAAAATAAACTGCTC |
| | | ACCCGTCTCCACCTG |
| SEQ ID | GI 169790843 NM_000239 Homo sapiens | CTTTGAAAGGTGTGA |
| NO: 1939 | lysozyme | GTTGGCCAGAACTCT |
| | (LYZ), mRNA | GAAAAGATTGGGAAT |
| | | GGATGGCTACAGGGG |
| SEQ ID | GI 740086238 NM_017921 Homo sapiens | CACCAGTCAGTTCTC |
| NO: 1940 | NPL4 homolog, ubiquitin | CCTTGAAGTAAACCT |
| | recognition factor | CAGTGCCTGAGACTT |
| | (NPLOC4), transcript variant 1, mRNA | TTCTACCAAGCCACA |
| SEQ ID | GI 1024336732 NM_006218 Homo sapiens | GCGAAATTCTCACAC |
| NO: 1941 | phosphatidylinositol-4,5-bisphosphate | TATTATGCAAATTCA |
| | 3-kinase catalytic subunit alpha | GTGCAAAGGCGGCTT |
| | (PIK3CA), mRNA | GAAAGGTGCACTGCA |
| SEQ ID | GI 365192584 NG_031951 Homo sapiens | CACACAAATACCCCA |
| NO: 1942 | prostate transmembrane | CAGCGTGAATATGCA |
| | protein, androgen induced 1 | CATGTACGTCCTGCA |
| | (PMEPA1), RefSeqGene on chromoso [symbol] | TATACATGCAACGCA |
| SEQ ID | GI 338968872 NG_029246 Homo sapiens | GTCTCCACTGGCCAA |
| NO: 1943 | RAR related orphan receptor A | GGTTGTGGTCCTCCT |
| | (RORA), RefSeqGene on chromosome 15 | TTCCAGTATTTTTCT |
| | | TGGATGACTACAGGA |
| SEQ ID | GI 190194362 NM_006426 Homo sapiens | CACCTAAGCTTCCAT |
| NO: 1944 | dihydropyrimidinase like 4 | GTAGCCCTCATCCAG |
| | (DPYSL4), mRNA | GGAAGTTTTGCGATC |
| | | CTTTAGGAAGACACT |
| SEQ ID | GI 58530885 NM_001570 Homo sapiens | ACACACAACAAAACA |
| NO: 1945 | interleukin 1 receptor | TCTGCTGTCCTGGGT |
| | associated kinase 2 | GGGAGGGAAACTTCA |
| | (IRAK2), mRNA | TTTCACTGGAATGAG |
| SEQ ID | GI 32891806 AY340485 Homo sapiens | AGACCCAGAGAGTGA |
| NO: 1946 | biliverdin reductase B | CTTGCCCAATATCAC |
| | (flavin reductase | ACAGCTGTTAACTGA |
| | (NADPH)) | CAGGACTGGGATTTG |
| | (BLVRB) gene, complete cds | |
| SEQ ID | GI 40288424 AY506357 Homo sapiens | CCACCTTGAGCCATC |
| NO: 1947 | truncated epidermal growth factor | ATGGGGGGAGTTGGA |
| | (beta-urogastrone) | CAGCATTTACTTGTC |
| | (EGF) gene, complete cds | AGTGAATTTGGTAAG |
| SEQ ID | GI 351542176 NG_030342 Homo sapiens | GAAGTGGGCCCATAA |
| NO: 1948 | collectin subfamily member 12 | AAGGCCCTCACATTT |
| | (COLEC12), RefSeqGene on chromosome 18 | TAGGAGTAAAACTGT |
| | | GGGCTAAGCACCTTT |
| SEQ ID | GI 197100280 NG_008374 Homo sapiens | GTGTGTTTCTCTGTA |
| NO: 1949 | cytochrome P450 family | CCTGCATTTGCACAT |
| | 11 subfamily B member 2 | GTGTAGTGTGTGCAC |
| | (CYP11B2), RefSeqGene on | GTGTCTGTGTGTGAA |
| | chromosome [symbol] | |
| SEQ ID | GI 223972657 NG_009817 Homo sapiens | CCTTCCTTGTTCCCT |
| NO: 1950 | E1A binding protein p300 | AGCCCCAATCTGGGA |
| | (EP300), RefSeqGene on chromosome 22 | TACATTCAATCAATC |
| | | TACAGGGTCTGCACA |
| SEQ ID | GI 293597500 NG_016966 Homo sapiens | CAGTGGCTAGCTAAG |
| NO: 1951 | phosphoglucomutase 1 | GATGGATATTTCAGG |
| | (PGM1), RefSeqGene on chromosome 1 | ATGGATCTGTGGAAT |
| | | GGTTACTGGGCCCAG |
| SEQ ID | GI 49066030 AY654286 Homo sapiens | TAGAATGGGACAAGA |
| NO: 1952 | interferon | TTAGGGTCAACCTCA |
| | (alpha, beta and omega) receptor 1 | GGCTGTCCTGTAGGC |
| | (IFNAR1) gene, complete cds | TTTTGGGGATTTTGT |
| SEQ ID | GI 440503012 JX512452 Homo sapiens | CTGAGGTAAGGGCCC |
| NO: 1953 | mitogen-activated protein kinase 14 | TGTGCTCCAGGAAAT |
| | (MAPK14) gene, complete cds | AGCTAAGAGACCAGT |
| | | ACAGCTAGAGCTAAA |
| SEQ ID | GI 212549766 NG_008985 Homo sapiens | ATTTTTCCTGACTGG |
| NO: 1954 | N-acylsphingosine amidohydrolase 1 | TTCCCAGAGTACAAA |
| | (ASAH1), RefSeqGene on chromosome 8 | GCACAGGACAACTTC |
| | | GCTAACTCACCCTGA |
| SEQ ID | GI 300193072 NG_023297 Homo sapiens | GTGTGTGACATTCCA |
| NO: 1955 | cyclin dependent kinase inhibitor 2B | GGGGTCGTTTGCTTT |
| | (CDKN2B), RefSeqGene on chromosome 9 | TCAGGGTTTTCTGAG |
| | | GGAAAGTGCATATGA |
| SEQ ID | GI 315360647 NG_028023 Homo sapiens | GACCTCTACAGCACC |
| NO: 1956 | conserved helix-loop-helix ubiquitous kinase | AGCATTTCCTGGAAG |
| | (CHUK), RefSeqGene on chromosome 10 | CTCTGCCCATAATTA |
| | | GTTTTGCAGAAGTGC |
| SEQ ID | GI 747813982 NG_007933 Homo sapiens | TTGAATCAGGGCTGG |
| NO: 1957 | cytochrome P450 family 2 | TGTCAGTTGGATCAG |
| | subfamily S member 1 | GGTGGTAGGGAAAGG |
| | (CYP2S1), RefSeqGene on chromosome 19 | AGGAAATGGATAAAT |
| SEQ ID | GI 284055256 NG_016403 Homo sapiens | TCTGACCTAAGAGCC |
| NO: 1958 | dystrophin related protein 2 | TGGAGCTGAAGGGCT |
| | (DRP2), RefSeqGene on chromosome X | CATCTCATCTCTGTA |
| | | TTTACTTCCTGGCTC |
| SEQ ID | GI 410991940 NG_032977 Homo sapiens | ATTGTTCAAGGAACA |
| NO: 1959 | interleukin 12 receptor subunit beta 2 | GTCTGAAGGCCAGTG |
| | (IL12RB2), RefSeqGene on chromosome 1 | TGAATGGAGCAGAGT |
| | | GAACCAGGCAAAAAT |
| SEQ ID | GI 212549709 NG_008958 Homo sapiens | TAGCAGAAGGTCAGC |
| NO: 1960 | minichromosome maintenance complex component 6 | TTTCACAGTTATTTC |
| | (MCM6), RefSeqGene on chromosom [symbol] | CCACTGAGAAGAGGC |
| | | GCTTTGATACTGCCA |
| SEQ ID | GI 312433967 NG_027936 Homo sapiens | CCGTACAATGTGGGC |
| NO: 1961 | pannexin 1 | ACCACCCATCTACAC |
| | (PANX1), RefSeqGene on chromosome 11 | CATTTCTCAAATTCT |
| | | CTGTGCATGCAGATC |
| SEQ ID | GI 237858748 NG_012115 Homo sapiens | GTCAGGAGGTCTTGA |
| NO: 1962 | phospholipid transfer protein | GAATTAGACTGGAGA |
| | (PLTP), RefSeqGene on chromosome 20 | GGGAATTTGGGCCTG |
| | | GTTCTTGGAAAACCT |
| SEQ ID | GI 747165391 NM_004590 Homo sapiens | TACTTAGCCAGTTTC |
| NO: 1963 | C-C motif chemokine ligand 16 | CAGCCTTCCAGATGA |
| | (CCL16), mRNA | TCTTTGCCCTCTGGG |
| | | TCTTGACCCATTAAG |
| SEQ ID | GI 974005372 NM_005944 Homo sapiens | ATTTGACACAAGAGA |
| NO: 1964 | CD200 molecule | AAAGCAGGAGGAAAA |
| | (CD200), transcript variant 1, mRNA | GGGGCCATTCTCCAA |
| | | AGGACCTGAAAGAGC |
| SEQ ID | GI 23110953 NM_001911 Homo sapiens | AGTCTTCACCAGGGT |
| NO: 1965 | cathepsin G | CTCAAGTTTCCTGCC |
| | (CTSG), mRNA | CTGGATAAGGACAAC |
| | | AATGAGAAGCTTCAA |
| SEQ ID | GI 440503012 JX512452 Homo sapiens | TGTTCCCTGCTCCCC |
| NO: 1966 | mitogen-activated protein kinase 14 | TTCCTCTTCTTGTGT |
| | (MAPK14) gene, complete cds | CTGTATCTCCAGCTA |
| | | CCATTTAGTCAGTCA |
| SEQ ID | GI 60391402 AB019488 Homo sapiens | GTATGCATTTGTGTG |
| NO: 1967 | NTRK1 gene for TRKA, complete cds | TGCACGCCTGTGTAA |
| | | GTGTGTATGCAGGTC |
| | | TGAACAGGTGCATAT |
| SEQ ID | GI 350606318 NG_009014 Homo sapiens | GTCAAAAAGACCGGC |
| NO: 1968 | androgen receptor | CCAATAGCTTCTCAG |
| | (AR), RefSeqGene on chromosome X | CGGGTATCCTCCAGA |
| | | GAGGTAAAGTGAAAT |
| SEQ ID | GI 329665040 NG_028340 Homo sapiens | CATACCTGCTAAAGA |
| NO: 1969 | granzyme B | AACTATCCTGATGGA |
| | (GZMB), RefSeqGene on chromosome 14 | TTTAGCAGCATGGCC |
| | | ATGAGGCATTGGCGG |
| SEQ ID | GI 257153317 NG_012804 Homo sapiens | TGAGAGGGCAAGGTC |
| NO: 1970 | mannosidase beta | TCTGGGAGGTAGGAG |
| | (MANBA), RefSeqGene on chromosome 4 | ATAAGAAGAGGATGG |
| | | ATTCACTTTGGGATG |
| SEQ ID | GI 237858770 NG_012124 Homo sapiens | CCTATACTGACCTGA |
| NO: 1971 | selectin E | GACAGAGGCAGCAGT |
| | (SELE), RefSeqGene on chromosome 1 | GATACCCACCTGAGA |
| | | GATCCTGTGTTTGAA |
| SEQ ID | GI 237858802 NG_012136 Homo sapiens | GACCACCATAAACTC |
| NO: 1972 | thrombopoietin | TGCTAGGGGATCTCT |
| | (THPO), RefSeqGene on chromosome 3 | AAAAAGGAGTCAGGC |
| | | TTATGGGGCTTTGCA |
| SEQ ID | GI 1036029829 AH003667 Homo sapiens | GAGGGTACTGAGGTA |
| NO: 1973 | chromosome 1 leptin receptor | ACCTATGAGGCCGAA |
| | (LEPR) gene, complete cds | AGCCAGAGACAACCC |
| | | TTTGTTAAATACGCC |
| SEQ ID | GI 520975386 NM_001278651 Homo sapiens | TGTCTCGAACCCTGG |
| NO: 1974 | Ran GTPase activating protein 1 | TCTAAAGTGATCTTC |
| | (RANGAP1), transcript variant 1, mRNA | CCGCCTAGCCTCCCA |
| | | AAATGTTGGAATTAC |
| SEQ ID | GI 313661479 NM_145725 Homo sapiens | AGCCCCCTTGCTTTT |
| NO: 1975 | TNF receptor associated factor 3 | GTATCTGTGAGGTGA |
| | (TRAF3), transcript variant 1, mRNA | ATGAGGGTCTGTCAC |
| | | CCAAATCTACTTCTC |
| SEQ ID | GI 117606567 EF064758 Homo sapiens | ATCGGAGACAACACC |
| NO: 1976 | chemokine | ACAGTGGACTACACT |
| | (C-C motif) receptor 7 | TTGTTCGAGTCTTTG |
| | (CCR7) gene, complete cds | TGCTCCAAGAAGGAC |
| SEQ ID | GI 117606505 EF064727 Homo sapiens | TTGAGGTTCGAATGA |
| NO: 1977 | inducible T-cell co-stimulator | TGTGCGATGTGTAAT |
| | (ICOS) gene, complete cds | GAGACAGAAGCCACT |
| | | GCATTTTTCCACCCC |
| SEQ ID | GI 38045925 AY449709 Homo sapiens | GGGCTGCATTTTATT |
| NO: 1978 | interleukin 7 receptor | GGGCCGGCAAGTAAC |
| | (IL7R) gene, complete cds | TTTAAATCCCAGCTC |
| | | ACACTCTACTGAGTG |
| SEQ ID | GI 254039585 NG_012387 Homo sapiens | CCCAGTTGAAGATGG |
| NO: 1979 | aldolase, fructose-bisphosphate B | GGAAACCAGGAAATG |
| | (ALDOB), RefSeqGene on chromosome 9 | TGCCATCAGATACTA |
| | | AGTGGAGTCAAGGGA |
| SEQ ID | GI 334085206 NG_029005 Homo sapiens | TGGCTGAACTGCTCT |
| NO: 1980 | CASP8 and FADD like apoptosis regulator | ACAGAGTGAGGCGAT |
| | (CFLAR), RefSeqGene on chromosome 2 | TTGACCTGCTCAAAC |
| | | GTATCTTGAAGATGG |
| SEQ ID | GI 1024846796 NG_046983 Homo sapiens | GAGGGCTTAATACCA |
| NO: 1981 | cAMP responsive element binding protein 3 | AGAGAACAAGTGAAG |
| | (CREB3), RefSeqGene on chromosome 9 | TGGGAGAAGCAGGGC |
| | | TAAACTTTCAGGGGA |
| SEQ ID | GI 577019604 NG_033916 Homo sapiens | AAATCACGTGGACCC |
| NO: 1982 | growth differentiation factor 2 | CTCTCATGACCTGAA |
| | (GDF2), RefSeqGene on chromosome 10 | AGGAAGCGTGGTCAT |
| | | TTATGATGTTCTGGA |
| SEQ ID | GI 342672038 NG_029624 Homo sapiens | CAAGACCAAATGGGA |
| NO: 1983 | NFKB inhibitor beta | TACCTGTCTCTTCCA |
| | (NFKBIB), RefSeqGene on chromosome 19 | GTCCCAGGACAAATG |
| | | TGGTTCAGAAGGGAT |
| SEQ ID | GI 105990527 NM_012258 Homo sapiens | ATCTGCCAGGACTGG |
| NO: 1984 | hes related family Bhlh | CAGTTCTGATTTAGT |
| | transcription factor with YRPW motif 1 | TAGGAGAGAGCCGCT |
| | (HEY1), transcript variant 1, | GATAGGTTAGGTCTC |
| SEQ ID | GI 1028916196 KU928257 Homo sapiens | GTACCTAATGCCCAG |
| NO: 1985 | complement decay-accelerating factor | CCAGCTTTGGAAGGC |
| | (CD55) gene, CD55-203A allele, | CGTACAAGTTTTCCC |
| | complete cds | GAGGATACTGTAATA |
| SEQ ID | GI 4588599 AF111163 Homo sapiens | TTACCCTTAGTCTCA |
| NO: 1986 | pyrin | GTTCCCACCAAGACA |
| | (MEFV) gene, complete cds | CAGAGCATTTCCTGT |
| | | GCCTTTTCCGCTATT |
| SEQ ID | GI 1048072306 NG_051267 Homo sapiens | TGTTTTAAGCAGGGC |
| NO: 1987 | arsA arsenite transporter, | AATGTCGTGGCCAAA |
| | ATP-binding, homolog 1 | TTCATGTTTGAAAAG |
| | (bacterial) | TACCCTTTGACGGCC |
| | (ASNAl), RefSeqGene on chr [symbol] | |
| SEQ ID | GI 334278891 NG_029019 Homo sapiens | TGTGAGTGGATACAT |
| NO: 1988 | CCAAT/enhancer binding protein beta | GTACAGCGTCCCACA |
| | (CEBPB), RefSeqGene on chromosome 20 | GTGCCTGGCACGCAA |
| | | GAAGTGTTATATAAG |
| SEQ ID | GI 291219938 NG_016779 Homo sapiens | AGCAGTCTACTAGTG |
| NO: 1989 | interleukin 2 | ACTTTACTGCCACAA |
| | (IL2), RefSeqGene on chromosome 4 | CCAGAAAAGCCAGCT |
| | | GATGTGTCAGACGTG |
| SEQ ID | GI 224809272 NG_011478 Homo sapiens | TGATGCCCAGAGAAG |
| NO: 1990 | uncoupling protein 2 | GGATACCATTTGCCT |
| | (UCP2), RefSeqGene on chromosome 11 | GAGCCTTCTCAGTAC |
| | | TGTTTAATCAGCCTG |
| SEQ ID | GI 112293258 NM_001736 Homo sapiens | AATTGTAAGTAATGA |
| NO: 1991 | complement C5a receptor 1 | TACAGAGGGATCTTG |
| | (C5AR1), mRNA | TGTACCCTTCACCCA |
| | | GCCTCCCCCAATGGC |
| SEQ ID | GI 1041817991 NM_000569 Homo sapiens | TTACTTCCTCCTGTC |
| NO: 1992 | Fc fragment of IgG receptor Illa | TAGTCGGTTTGGTCC |
| | (FCGR3A), transcript variant 1, mRNA | CTTTAGGGCTCCGGA |
| | | TATCTTTGGTGACTT |
| SEQ ID | GI 571330780 NM_001136022 Homo sapiens | ACTGGAGACGCGGCC |
| NO: 1993 | nuclear factor of activated T-cells 4 | TCTAAGAGAGGTTGA |
| | (NFATC4), transcript variant 1, mRNA | AACTGTGTGTGTGTG |
| | | GGAGAAAATGATAAC |
| SEQ ID | GI 21634442 AF274025 Homo sapiens | CTGAGGAAGATTATA |
| NO: 1994 | GTP-binding protein Rab25 | ACTTTGTCTTCAAGG |
| | mRNA, complete cds | TGGTGCTGATCGGCG |
| | | AATCAGGTGTGGGGA |
| SEQ ID | GI 256017191 NM_002977 Homo sapiens | AGGCCTGTTTCACAG |
| NO: 1995 | sodium voltage-gated channel | ATGGTTGTGTACGGA |
| | alpha subunit 9 | GGTTCTCATGCTGCC |
| | (SCN9A), mRNA | AAGTTAACATAGAGT |
| SEQ ID | GI 384551644 NM_005428 Homo sapiens | TTACCAGCAGAACTC |
| NO: 1996 | vav guanine nucleotide exchange factor 1 | TCTAAAGGATTGCTT |
| | (VAV1), transcript variant 1, mRNA | CAAGTCTCTGGACAC |
| | | CACCTTGCAGTTCCC |
| SEQ ID | GI 117606495 EF064722 Homo sapiens | CCACAACAGCATTCA |
| NO: 1997 | interleukin 6 signal transducer | GGACTGTAGAGGAGA |
| | (gpl30, oncostatin M receptor) | CCACCACTGCCTGTT |
| | (II6ST) gene, complete cds | TTCTGGAGAGTTTAG |
| SEQ ID | GI 83026500 DQ309025 Homo sapiens | TTTTTCTCCTATTAA |
| NO: 1998 | transforming growth factor | AGGGCCTTGAGGGAT |
| | beta 1 induced transcript 1 | GGAGCTCTATTCAGA |
| | (TGFBIIl) gene, complete cds | GTGCCAGCTGTGCTC |
| SEQ ID | GI 269914110 NG_015981 Homo sapiens | CACATTGGTTTCCTC |
| NO: 1999 | anti-Mullerian hormone receptor type 2 | CTGAGAAGAGTCAAC |
| | (AMHR2), RefSeqGene on chromosome 12 | AGAGTCCAGCATCTT |
| | | CTTCCAAGGTCAGGG |
| SEQ ID | GI 347658933 NG_030027 Homo sapiens | TTCTTTCTTCTCTTC |
| NO: 2000 | AT-rich interaction domain 5B | ATTTAGGGTCACACA |
| | (ARID5B), RefSeqGene on chromosome 10 | CACTCCGGGACCATG |
| | | GAGGGAGGTATCAGA |
| SEQ ID | GI 349732110 NG_030313 Homo sapiens | TCGAAGGAGGCACTC |
| NO: 2001 | dihydrolipoamide S-succinyltransferase | CACTTTTCACACTCA |
| | (DLST), RefSeqGene on chromosome 14 | GGAAAACTGGTGGTA |
| | | AAGAAGTTCTCCTGG |
| SEQ ID | GI 344217739 NG_029805 Homo sapiens | CATTCCCGAGCTCAT |
| NO: 2002 | DnaJ heat shock protein family | GTACCGCACTCAAGA |
| | (Hsp40) member C5 | TCAAGGATACACTGG |
| | (DNAJC5), RefSeqGene on chromosome [symbol] | ACATTGAAGGGTGAG |
| SEQ ID | GI 767172199 NG_042051 Homo sapiens | GGTGACTTAGATGTG |
| NO: 2003 | kelch like family member 41 | TGTTAAAGTTGAAGG |
| | (KLHL41), RefSeqGene on chromosome 2 | CCGCCGATCTAGAGA |
| | | AACGTGGCTGCCTAT |
| SEQ ID | GI 342187223 NG_029593 Homo sapiens | TTCCTCTTGTTTGTG |
| NO: 2004 | LYN proto-oncogene, | TGATCCTTGTGACAT |
| | Src family tyrosine kinase | CTGACTTGGGTGACA |
| | (LYN), RefSeqGene on chromosome 8 | TGCTTCCTCTGTGGA |
| SEQ ID | GI 237858739 NG_012110 Homo sapiens | ATGGAATCCCATAGA |
| NO: 2005 | programmed cell death 1 | AACACCCCAAATCTA |
| | (PDCD1), RefSeqGene on chromosome 2 | ACTTGATCCAGGACC |
| | | AGCCAGTGGTCACTT |
| SEQ ID | GI 1018443413 NG_027814 Homo sapiens | GTAGAGATTTTTTGA |
| NO: 2006 | SATB homeobox 1 | GTTTCAGTCTCCACT |
| | (SATB1), RefSeqGene on chromosome 3 | TGGGAAGGTTCCTTG |
| | | GCCCCTTCTCTCCCA |
| SEQ ID | GI 22035651 NM_014349 Homo sapiens | CAGGCATCTTCCTTG |
| NO: 2007 | apolipoprotein L3 | CACTGGATGTGGTCA |
| | (APOL3), transcript | ACCTTGTATACGAGT |
| | variant alpha/a, mRNA | CAAAGCACTTGCATG |
| SEQ ID | GI 2909349 Y12863 Homo sapiens | GCGAGTGTGTCTGTA |
| NO: 2008 | mRNA for growth factor FIGF | AAACACCATGTCCCA |
| | | AAGATCTAATCCAGC |
| | | ACCCCAAAAACTGCA |
| SEQ ID | GI 378404896 NM_001256796 Homo sapiens | GAGGAGGGAGAAATG |
| NO: 2009 | purinergic receptor P2X 4 | GCCACCACATCACCC |
| | (P2RX4), transcript variant 1, mRNA | CAGAGAAATTTCTGG |
| | | AATCTGATTGAGTCT |
| SEQ ID | GI 998614224 NM_001320340 Homo sapiens | GGGAGCTCTTTAGTC |
| NO: 2010 | small VCP interacting protein | GTCTAGGAGATGAAG |
| | (SVIP), transcript variant 1, mRNA | AGAAAAGAGCAAAGC |
| | | TTGCAGAGGCTGCAG |
| SEQ ID | GI 115432768 DQ995342 Homo sapiens | AATGATCATAGTGTC |
| NO: 2011 | ADAM metallopeptidase domain 33 | CGCTTCAGGGTGGTT |
| | (ADAM33) gene, complete cds | TATGAGGCTTAAAGG |
| | | GAAGAAGCTCAGGCA |
| SEQ ID | GI 87046146 DQ142911 Homo sapiens | CACATCTCATATGCA |
| NO: 2012 | cogulation factor VII | CAAGACACCTCACAC |
| | (F7) gene, complete cds | TCAGGACACCTCATG |
| | | CTCAAAGAAGCCTCA |
| SEQ ID | GI 114731580 DQ993361 Homo sapiens | GTGAGTGTGATTCTA |
| NO: 2013 | glutathione S-transferase A3 | GAAGAGGATGCATTC |
| | (GSTA3) gene, complete cds | CATGGATCCAGCACC |
| | | CTGACAGAGGTTTCC |
| SEQ ID | GI 345525419 NG_029928 Homo sapiens | TTGCCACCACTATAA |
| NO: 2014 | CD86 molecule | CCTCATCATGGCTCA |
| | (CD86), RefSeqGene on chromosome 3 | CCTTCCTGCTGCTTC |
| | | TCAACTACTGTTCTG |
| SEQ ID | GI 270289740 NG_015895 Homo sapiens | CTCTGGGTGGCTGAT |
| NO: 2015 | four and a half LIM domains 1 | TAGCTGAGGGTAACC |
| | (FHL1), RefSeqGene on chromosome X | TTCATATAGCTGGCA |
| | | TGGGATTTCCAGATG |
| SEQ ID | GI 226693358 NG_011855 Homo sapiens | GGAGTCTCATGCACC |
| NO: 2016 | heat shock protein family A | ACCTATCACACTCCC |
| | (Hsp70) member 1 like | TCACATCCTGTTTCT |
| | (HSPAlL), RefSeqGene on chromosome [symbol] | GACTTTGGAATGGAC |
| SEQ ID | GI 224809277 NG_011481 Homo sapiens | ACAATGGAGTGGGAT |
| NO: 2017 | interleukin 18 receptor accessory protein | TGAGACTGTGGTTTA |
| | (IL18RAP), RefSeqGene on chromosome 2 | GAGCCTTTGATTTCC |
| | | TGGACTGGACTGACG |
| SEQ ID | GI 237858713 NG_012098 Homo sapiens | AAAACTGGCAGCAGC |
| NO: 2018 | KIT ligand | AAGTACAAAGGGCTT |
| | (KITLG), RefSeqGene on chromosome 12 | GAGGCAGGAATTACC |
| | | TTCCAGGTATTTCAG |
| SEQ ID | GI 226493744 NG_011821 Homo sapiens | AAATCACTGCCACCA |
| NO: 2019 | nuclear receptor subfamily 4 group A member 2 | CTTCTCTCCCCAGCT |
| | (NR4A2), RefSeqGene on chromosome 2 | TCAGTACCTTTATGG |
| | | ACAACTACAGCACAG |
| SEQ ID | GI 1027250682 NG_047124 Homo sapiens | AGGACCTCTATTCTG |
| NO: 2020 | solute carrier family 27 member 5 | TAGGGGCTAAGGTGG |
| | (SLC27A5), RefSeqGene on chromosome 19 | GCTGCATATATAACC |
| | | AGCACTTGGTTCTCT |
| SEQ ID | GI 425905339 NM_080725 Homo sapiens | CCAGTTCCCACCTCT |
| NO: 2021 | sulfiredoxin 1 | GTGACAATAGGATCA |
| | (SRXN1), mRNA | TGGCCTTACCCTTGA |
| | | AGCATTACCGAGAAG |
| SEQ ID | GI 117606531 EF064740 Homo sapiens | CGGGTGCATTGAATA |
| NO: 2022 | Epstein-Barr virus induced gene 3 | AGAGCAGTCCAGCAA |
| | (EBI3) gene, complete cds | TCTGATTCCTAGGCC |
| | | TCTACCGAAAAGGAT |
| SEQ ID | GI 39654020 AY496422 Homo sapiens | AGAGAGTGAGGATGA |
| NO: 2023 | nuclear factor of kappa light polypeptide | GGAGAGCTATGACAC |
| | gene enhancer in B-cells inhibitor, alpha | AGAGTCAGAGTTCAC |
| | (NFKBIA) gen | GGAGTTCACAGAGGA |
| SEQ ID | GI 7417326 AF237581 Homo sapiens | GTGCTCACAGTTACA |
| NO: 2024 | migration inhibitory factor- | CAGGACGACAGGGTC |
| | related protein 14 variant P | AAGAATTTGCTCAAT |
| | (S100A9) gene, complete cds | TGAACACCTGCCATT |
| SEQ ID | GI 662236863 NG_034192 Homo sapiens | GAATGAAGGTGTTTC |
| NO: 2025 | diacylglycerol O-acyltransferase 1 | TAGAGTCAGAGTCCT |
| | (DGAT1), RefSeqGene on chromosome 8 | CAGTGAACCCTGGCA |
| | | TCCTGAGATCCAGGA |
| SEQ ID | GI 1024846743 NG_047006 Homo sapiens | GGAGCTCACTGCCCC |
| NO: 2026 | hexokinase 3 | TGAAGGCAACAGTTG |
| | (HK3), RefSeqGene on chromosome 5 | CATTCTTAGACACCT |
| | | GTGATCAATGAAAAG |
| SEQ ID | GI 654825051 NG_029042 Homo sapiens | CTTTTGGAAAGAGAG |
| NO: 2027 | prolactin receptor | GGCCTAAACAGGATC |
| | (PRLR), RefSeqGene on chromosome 5 | AGCATTGATGCCAAG |
| | | CAACACAAGGGAGAA |
| SEQ ID | GI 237858601 NG_012145 Homo sapiens | CAGTAATTCCATGAG |
| NO: 2028 | TNF receptor superfamily member 10b | CACAGGCCCATTATA |
| | (TNFRSF10B), RefSeqGene on chromosome 8 | GTGGGCGCTTCATTT |
| | | GCCATCAAGTGAGTC |
| SEQ ID | GI 260763875 NM_182569 Homo sapiens | CAGGTTTGAACCACT |
| NO: 2029 | glycerophosphodiester phosphodiesterase | GCACCCGGCCAGTAA |
| | domain containing 1 | AAGAAATTTTGAAGG |
| | (GDPD1), transcript variant [symbol] | CCATTGCAGCTATTT |
| SEQ ID | GI 209413750 NM_006038 Homo sapiens | GATGGTCTCCTTTGA |
| NO: 2030 | spermatogenesis associated 2 | GCTCTTTCTGGCCAA |
| | (SPATA2), transcript variant 1, mRNA | AGTCGAGTGTGAGCA |
| | | GATGCTAGAAATCCA |
| SEQ ID | GI 117606569 EF064759 Homo sapiens | TTCTCCAGAGGGAAT |
| NO: 2031 | chemokine | TGCAGAGTACTGGCT |
| | (C-C motif) receptor 4 | GATGGAGTAAATCGC |
| | (CCR4) gene, complete cds | TACCTTTTGCTGTGG |
| SEQ ID | GI 74418831 DQ185447 Homo sapiens | GCCCAAGGTTCAGAG |
| NO: 2032 | interferon, alpha 1 | TCACCCATCTCAGCA |
| | (IFNA1) gene, complete cds | AGCCCAGAAGTATCT |
| | | GCAATATCTACGATG |
| SEQ ID | GI 46488017 AY601851 Homo sapiens | TAAACAGTGATTGTG |
| NO: 2033 | mutS homolog 2, colon | ACCAGGGACATAGAA |
| | cancer, nonpolyposis type 1 | TGGGATGATTGGCCC |
| | (E. coli) | GAGGTAACCATGGTT |
| | (MSH2) gene, complete cds | |
| SEQ ID | GI 654825051 NG_029042 Homo sapiens | CTCCCAGGGGAATTT |
| NO: 2034 | prolactin receptor | GGCAATGTCTGGAGA |
| | (PRLR), RefSeqGene on chromosome 5 | CACTTTTTATTGTCA |
| | | TGACTGGGGAGTTGC |
| SEQ ID | GI 237858776 NG_012127 Homo sapiens | AGGCGAAGGAGATAG |
| NO: 2035 | solute carrier family 2 member 4 | AAGGAGAGGCAGGAG |
| | (SLC2A4), RefSeqGene on chromosome 17 | AGATAGGTAAGGCCA |
| | | GAGATCGGATAAGAG |
| SEQ ID | GI 224967082 NG_011581 Homo sapiens | GAAGGTCTGAGACAC |
| NO: 2036 | thyroid peroxidase | CCATCGGCCAACCCT |
| | (TPO), RefSeqGene on chromosome 2 | AACTCATCCTGTCTT |
| | | CTCTCTCTAGTTCAC |
| SEQ ID | GI 299758401 NM_000143 Homo sapiens | GTCTGGGAGAATTGA |
| NO: 2037 | fumarate hydratase | TCTTGCCTGAAAATG |
| | (FH), mRNA | AACCAGGAAGCAGTA |
| | | TCATGCCAGGCAAGG |
| SEQ ID | GI 1002635078 NM_006256 Homo sapiens | CATGTTTCCAAACCA |
| NO: 2038 | protein kinase N2 | AAGAGCATGTTTGCT |
| | (PKN2), transcript variant 1, mRNA | TTGTAATGGAATATG |
| | | CTGCCGGTGGGGACC |
| SEQ ID | GI 21927971 AY113189 Homo sapiens | TCACTCAGGGTGATG |
| NO: 2039 | OCT-1 protein | TTGGGCTCGCTATGG |
| | (POU2F1) mRNA, complete cds | GGAAACTATATGGAA |
| | | ATGACTTCAGCCAAA |
| SEQ ID | GI 256017191 NM_002977 Homo sapiens | AAGAAGGGCCAACTA |
| NO: 2040 | sodium voltage-gated | AATTAGGATGGCCAG |
| | channel alpha subunit 9 | GGAATTGGCCTGGGT |
| | (SCN9A), mRNA | TTAGGACCTATGTAT |
| SEQ ID | GI 1049011036 AH002959 Homo sapiens | CAGTCGGATGTCTAC |
| NO: 2041 | RAF1 | TCCTATGGCATCGTA |
| | (RAF1) gene, complete cds | TTGTATGAACTGATC |
| | | ACGGGGGAGCTTCCT |
| SEQ ID | GI 237820683 NG_012068 Homo sapiens | AAATATAGCGAAATC |
| NO: 2042 | forkhead box 11 | TCTCCACTGCCTTCT |
| | (FOXIl), RefSeqGene on chromosome 5 | GTCCACCAACATTTA |
| | | GTGCCAGCCTAGGCA |
| SEQ ID | GI 211064488 NG_008914 Homo sapiens | AAATCCAAGTGACCA |
| NO: 2043 | heat shock protein family E | GGGAAGTTAAAAGTA |
| | (HsplO) member 1 | TTCCTGACGGTGCAA |
| | (HSPE1), RefSeqGene on chromosome 2 | AAAGCCGCTCAGCAA |
| SEQ ID | GI 225735577 NG_011719 Homo sapiens | GTCATCACGGATGGA |
| NO: 2044 | integrin subunit alpha M | GAAAAGTTTGGCGAT |
| | (ITGAM), RefSeqGene on chromosome 16 | CCCTTGGGATATGAG |
| | | GATGTCATCCCTGAG |
| SEQ ID | GI 757813200 NG_012843 Homo sapiens | GTCCTGGGTCATTCT |
| NO: 2045 | lipin 1 | CTTCCAAACAGGAGA |
| | (LPIN1), RefSeqGene on chromosome 2 | TCCATCATTGTCACG |
| | | GCTCTGCAGTGTAAT |
| SEQ ID | GI 908212454 NG_042284 Homo sapiens | TTCTGTGAATCCCGG |
| NO: 2046 | SMAD family member 1 | AGTCTGCAAATGAGT |
| | (SMAD1), RefSeqGene on chromosome 4 | TATAGAAAGCTGTTG |
| | | AGGCCTGAGGTGCTT |
| SEQ ID | GI 298160932 NG_021435 Homo sapiens | TAGTAGGCTTGCAGC |
| NO: 2047 | vesicle associated membrane protein 7 | CCCAGTTGGTTAGCA |
| | (VAMP7), RefSeqGene on chromosome X and Y | TGACTAACAGTCCAC |
| | | AGAATGAGTTGGATG |
| SEQ ID | GI 189339276 NM_001204 Homo sapiens | TAATGGCAGTAACAG |
| NO: 2048 | bone morphogenetic protein receptor type 2 | GGCAGTTCATTCCAA |
| | (BMPR2), mRNA | ATCCAGCACTGCTGT |
| | | TTACCTTGCAGAAGG |
| SEQ ID | GI 558472802 NM_001042600 Homo sapiens | GGCACTGAAGATGGT |
| NO: 2049 | mitogen-activated protein | GAAGATGGAGCCTGA |
| | kinase kinase kinase kinase 1 | TGATGATGTCTCCAC |
| | (MAP4K1), transcript variant 1, | CCTTCAGAAGGAAAT |
| SEQ ID | GI 19072753 AF475085 Homo sapiens | TTACTTAGGTTACTG |
| NO: 2050 | mucolipin-3 | CTTCTGTGGATGGAT |
| | (MCOLN3) mRNA, complete cds | CGTGCTGGGGCCTTA |
| | | CCATGACAAGTTTCG |
| SEQ ID | GI 570359552 NM_001288718 Homo sapiens | TTTCACTCCGGAGTG |
| NO: 2051 | signal transducer and | AGAAGCTTTGCCCTC |
| | activator of transcription 5A | CTAAGAGAGAGAGAC |
| | (STAT5A), transcript variant 1, mRNA [symbol] | AGAGAGACAGAGAGA |
| SEQ ID | GI 269846911 NM_001167942 Homo sapiens | CTTCAAGAACGTACA |
| NO: 2052 | TNF alpha induced protein 8 like 1 | TGGATCCTGATTTTC |
| | (TNFAIP8L1), transcript variant 1, mRNA | TCACGAAGTCCCGAT |
| | | GGAACCCTGTGCTGT |
| SEQ ID | GI 577019603 NG_033914 Homo sapiens | CAGGCCCCAGACTGT |
| NO: 2053 | CD207 molecule | GTAAATGTCTGAGTG |
| | (CD207), RefSeqGene on chromosome 2 | GCATGATCAGCTATC |
| | | AAGTCTCCAAGTCAC |
| SEQ ID | GI 818213488 NG_042142 Homo sapiens | TTTTGTGCCTGTTTG |
| NO: 2054 | inositol 1,4,5-trisphosphate receptor type 2 | CCTTTGCTCATCCTG |
| | (ITPR2), RefSeqGene on chromosome 12 | TGTTATCTGCCTGGA |
| | | ATGTCTTCCCTCATT |
| SEQ ID | GI 285026427 NG_016458 Homo sapiens | TGTTCTTCCTCCTTT |
| NO: 2055 | protein kinase AMP-activated | CCCCAGGTGTGTTTC |
| | non-catalytic subunit gamma 3 | CCTCACCTCCAATTT |
| | (PRKAG3), RefSeqGene on chr [symbol] | CCTCTTTTCAGAAGT |
| SEQ ID | GI 345842421 NG_029949 Homo sapiens | AAAGAAACTGCCCTT |
| NO: 2056 | TNF superfamily member 13 | GTTGGGCCCTCAAAG |
| | (TNFSF13), RefSeqGene on chromosome 17 | GACAGCCCTGGTGGT |
| | | ATTAAAATAAAGACG |
| SEQ ID | GI 219521851 NG_009233 Homo sapiens | ACTCATTGCTCTCAA |
| NO: 2057 | tyrosine 3-monooxygenase/tryptophan | ATTACCTGTCTGTCT |
| | 5-monooxygenase activation protein epsilon | TCACTGCTCCTCTCA |
| | (YWHA [symbol] | GGAGACCCCTATGCT |
| SEQ ID | GI 164698262 AK314515 Homo sapiens | CCTACAAAAAATGGC |
| NO: 2058 | cDNA, FLI95334, Homo sapiens | GTGGAACCAAAGCGA |
| | protein inhibitor of activated STAT, 1 | CCCAGCCGACCAATT |
| | (PIAS1), mRNA | AATATCACCTCACTT |
| SEQ ID | GI 2304970 U82828 Homo sapiens | AAATGGTGAGCAGAG |
| NO: 2059 | ataxia telangiectasia | GACTCATTTCCCAAA |
| | (ATM) gene, complete cds | GACAGCTGGAACAGA |
| | | AATAGGCACTCCAGA |
| SEQ ID | GI 148733545 EF636667 Homo sapiens | CTGTTGTCTTTGATC |
| NO: 2060 | caspase 4, apoptosis- | CACTCCAGGTGGGGA |
| | related cysteine peptidase | GGGACGACCTCAGAA |
| | (CASP4) gene, complete cds | CAGTAATTCACAATG |
| SEQ ID | GI 57165045 AY864066 Homo sapiens | ATCGTATGGGACCTT |
| NO: 2061 | cyclin B2 | TGGCACTTGATTTAG |
| | (CCNB2) gene, complete cds | TCTGGTCTCAGGGCC |
| | | AGAATCATCTTGCAT |
| SEQ ID | GI 83026426 DQ301957 Homo sapiens | AGGCAGACATCAGCT |
| NO: 2062 | dual specificity phosphatase 1 | CCTGGTTCAACGAGG |
| | (DUSP1) gene, complete cds | CCATTGACTTCATAG |
| | | GTAAATGGAATGGAT |
| SEQ ID | GI 1036032759 AH002916 Homo sapiens | AGGTTTGCTGTACTT |
| NO: 2063 | ornithine aminotransferase | AGTCGCGGAGTTCAT |
| | (OAT) gene, complete cds | TCTTCAGTGGCTTCT |
| | | GCTACATCTGTTGCA |
| SEQ ID | GI 21105786 AF512504 Homo sapiens | GAGACTGGAGTATGT |
| NO: 2064 | X-ray repair complementing defective | GATGATGCCACACAC |
| | repair in Chinese hamster cells 1 | TAAAGGTGTGCTTCT |
| | (XRCC1) gene, complete | TGCCCAGGTAACTGT |
| SEQ ID | GI 189571639 NG_007991 Homo sapiens | TGATGACACTGGGTG |
| NO: 2065 | acyl-CoA dehydrogenase, | ACTCTGATCATCCAG |
| | C-2 to C-3 short chain | AATAACCCTCACATC |
| | (ACADS), RefSeqGene on chromosome 12 | TCCAGACGCTTCACT |
| SEQ ID | GI 184160977 NG_007553 Homo sapiens | GGGAAGAATTGATTG |
| NO: 2066 | actin, alpha, cardiac muscle 1 | TTGGATGGCTCCCAC |
| | (ACTC1), RefSeqGene on chromosome 15 | CTTCCAGCTCAGGAG |
| | | TTTTGATTTGACAGG |
| SEQ ID | GI 289666799 NG_016606 Homo sapiens | CTGTCAGCTGAAGTA |
| NO: 2067 | glycerol-3-phosphate dehydrogenase 2 | GGATGGCTTTAGCTC |
| | (GPD2), RefSeqGene on chromosome 2 | AGGGGGCTACTCAAC |
| | | ATTTGCTGTCATCTT |
| SEQ ID | GI 291084780 NG_016747 Homo sapiens | CAGGAACTGCCTTAT |
| NO: 2068 | PPARG coactivator 1 beta | TAACCCAGTCCCTCA |
| | (PPARGC1B), RefSeqGene on chromosome 5 | TGAAAATGGAGGTGA |
| | | AATTGGCCTGCTGTG |
| SEQ ID | GI 908673232 NG_042289 Homo sapiens | CTCTTACCAGATTTC |
| NO: 2069 | sodium voltage-gated channel | AATGGAGATGCTGGA |
| | alpha subunit 3 | GGATTCCTCTGGAAG |
| | (SCN3A), RefSeqGene on chromosome 2 | GCAAAGAGCCGTGAG |
| SEQ ID | GI 1027250680 NG_047105 Homo sapiens | AGGCAATTTGATTTA |
| NO: 2070 | slit guidance ligand 2 | GATGAGGTAATGGGC |
| | (SLIT2), RefSeqGene on chromosome 4 | CAGGGGCTCTCATGA |
| | | TGGGATCGATGTCTT |
| SEQ ID | GI 197313788 NM_001423 Homo sapiens | TTCCTGGTTAAGCAG |
| NO: 2071 | epithelial membrane protein 1 | GCATTGCTTTGCCCT |
| | (EMP1), mRNA | GGAGCAGCTATTTTA |
| | | AGCCATCTCAGATTC |
| SEQ ID | GI 38016940 NM_014365 Homo sapiens | GTTCTGCTTCTCCCT |
| NO: 2072 | heat shock protein family B | GCAGAAAAGCAGCAT |
| | (small) member 8 | TTTCGGAAGCTGAAG |
| | (HSPB8), mRNA | AATAAGCTAGCCCAG |
| SEQ ID | GI 93009032 DQ464902 Homo sapiens | CCAAAACTTGGCAAG |
| NO: 2073 | nexilin | GGTGATGTAAAGGAT |
| | (NEXN) mRNA, complete cds | AAGTTTGAAGCCATG |
| | | CAGAGAGCCAGGGAA |
| SEQ ID | GI 21927971 AY113189 Homo sapiens | CAACAACTGCAACAG |
| NO: 2074 | OCT-1 protein | CTTCAACAGCAGAAT |
| | (POU2F1) mRNA, complete cds | CTCAACCTGCAACAG |
| | | TTTGTGTTGGTGCAT |
| SEQ ID | GI 375151559 NM_005651 Homo sapiens | AAAGACACTTCTGGA |
| NO: 2075 | tryptophan 2,3-dioxygenase | ATTAGTGGAGGCATG |
| | (TD02), mRNA | GCTGGAAAGAACTCC |
| | | AGGTTTAGAGCCACA |
| SEQ ID | GI 1036032812 AH005421 Homo sapiens | TGAAATCTGAAGACC |
| NO: 2076 | CD19 | TCGAGCAGATGATGC |
| | (CD19) gene, complete cds | CAACCTCTGGAGCAA |
| | | TGTTGCTTAGGATGT |
| SEQ ID | GI 267844874 NG_015840 Homo sapiens | TCTGTATGTCATGCT |
| NO: 2077 | interferon gamma | TCTTCCGTAGGTTTG |
| | (IFNG), RefSeqGene on chromosome 12 | GCTTGCATGATGTCA |
| | | GGAGTTCCGGAAGTC |
| SEQ ID | GI 194018568 NG_008185 Homo sapiens | GAATGCAGTGCAGGG |
| NO: 2078 | lactate dehydrogenase A | TTGGATTAACAAGGT |
| | (LDHA), RefSeqGene on chromosome 11 | GGAAAGAATTCGAGG |
| | | GTTCCACCAAGTAGC |
| SEQ ID | GI 342307076 NG_029608 Homo sapiens | AGGGTATAGGCTTAA |
| NO: 2079 | phospholipase D2 | GGTCAGGAAGACCTG |
| | (PLD2), RefSeqGene on chromosome 17 | GACCCAAATCCTGGA |
| | | CTCCTCAATGAGAAG |
| SEQ ID | GI 364023822 NM_130898 Homo sapiens | GTCTCTTGACTCTTT |
| NO: 2080 | cAMP responsive element | CCGCCTTTGTTTACA |
| | binding protein 3 like 4 | ACCCTGCCATGATCT |
| | (CREB3L4), transcript variant 1, mRNA | CCCTCTTGCAAAAGC |
| SEQ ID | GI 227430281 NM_182983 Homo sapiens | CCCATAATCAGCAAT |
| NO: 2081 | hepsin | GATGTCTGCAATGGC |
| | (HPN), transcript variant 1, mRNA | GCTGACTTCTATGGA |
| | | AACCAGATCAAGCCC |
| SEQ ID | GI 117606585 EF064767 Homo sapiens | AGAAGGTCCATGTGG |
| NO: 2082 | chemokine | CTGGGACAAAATGAT |
| | (C-C motif) ligand 17 | ATAGTGGGTGATGGA |
| | (CCL17) gene, complete cds | GCTGGAGGAAAAAGT |
| SEQ ID | GI 219842221 NG_009291 Homo sapiens | TGTGTGTTGCATGAG |
| NO: 2083 | albumin | AAAACGCCAGTAAGT |
| | (ALB), RefSeqGene on chromosome 4 | GACAGAGTCACCAAA |
| | | TGCTGCACAGAATCC |
| SEQ ID | GI 798958230 NG_042077 Homo sapiens | GATGTGTGATATCAT |
| NO: 2084 | enoyl-CoA hydratase, short chain 1 | CTATGCCGGTGAGAA |
| | (ECHS1), RefSeqGene on chromosome 10 | GGCCCAGTTTGCACA |
| | | GCCGGAGATCTTAAT |
| SEQ ID | GI 336285267 NG_029154 Homo sapiens | TGGAGCCTAAGGTTT |
| NO: 2085 | interferon alpha 2 | AGGCTCACCCATTTC |
| | (IFNA2), RefSeqGene on chromosome 9 | AACCAGTCTAGCAGC |
| | | ATCTGCAACATCTAC |
| SEQ ID | GI 684179348 NG_034249 Homo sapiens | CCCACTTTACAGAGG |
| NO: 2086 | transmembrane protein 173 | AGGACACTGAGGCTC |
| | (TMEM173), RefSeqGene on chromosome 5 | AGAGACACCGTAGGA |
| | | AAGTGGACGATTTAA |
| SEQ ID | GI 217416377 NM_138394 Homo sapiens | TGGCTATGGATCCCA |
| NO: 2087 | heterogeneous nuclear | TGGTCCATTATTGCC |
| | ribonucleoprotein L like | TTTACCAAGTCGTTA |
| | (HNRNPLL), transcript variant 1, mRNA | CAGAATGGGCTCTCG |
| SEQ ID | GI 187763976 EU518415 Homo sapiens | ATGCTGTATGGCCTG |
| NO: 2088 | serum/glucocorticoid | CCGCCTTTTTATAGC |
| | regulated kinase 1 isoform 2 | CGAAACACAGCTGAA |
| | (SGK1) mRNA, complete cds | ATGTACGACAACATT |
| SEQ ID | GI 41349444 NM_014011 Homo sapiens | GTAAACTCTCCGGTC |
| NO: 2089 | suppressor of cytokine signaling 5 | CCCAAAGGTTGTTAA |
| | (SOCS5), transcript variant 1, mRNA | CTAGGTCCGCTTTCA |
| | | TGTGCATCAGACAGT |
| SEQ ID | GI 356460958 NG_030484 Homo sapiens | AAAACAGAAAGAACC |
| NO: 2090 | adenosine kinase | CAGTGGCCCAACTGA |
| | (ADK), RefSeqGene on chromosome 10 | ATGGAGACAAAGCCA |
| | | AGGTGGCTAGAATTT |
| SEQ ID | GI 259013220 NG_012921 Homo sapiens | TTCTGCTAGGCTAGC |
| NO: 2091 | bisphosphoglycerate mutase | AAAAGGGAGAATAGC |
| | (BPGM), RefSeqGene on chromosome 7 | CTAGGTCCGTGGATG |
| | | ATGTTTTGATGCACG |
| SEQ ID | GI 379642970 NG_032137 Homo sapiens | TGACATTCAGAAAGG |
| NO: 2092 | collagen type IV alpha 2 chain | CTGACCCAGCTTTGG |
| | (COL4A2), RefSeqGene on chromosome 13 | AACTCTGCTTGGTTT |
| | | GTTTGTTCGTTCCTT |
| SEQ ID | GI 336391122 NG_029141 Homo sapiens | TATTTGACATCCTGG |
| NO: 2093 | interferon gamma inducible protein 16 | GGTTACAGCACGCTA |
| | (IFI16), RefSeqGene on chromosome 1 | AAGGGCACCCACTGT |
| | | CCTTAACAGAGAAAA |
| SEQ ID | GI 224809429 NG_011538 Homo sapiens | CACCTCTACATCCTC |
| NO: 2094 | potassium voltage-gated | TGCATTCTTGGCCAG |
| | channel interacting protein 1 | GCTCTGAGAGGGCAT |
| | (KCNIP1), RefSeqGene on chromoso [symbol] | TGGTAAATGTTAACT |
| SEQ ID | GI 269954733 NG_016014 Homo sapiens | AACCACCAGCACCTT |
| NO: 2095 | membrane spanning 4-domains A2 | GTGCTCATGTACTTG |
| | (MS4A2), RefSeqGene on chromosome 11 | CCCATGTCTGATGGA |
| | | AGAAACATACTCTCT |
| SEQ ID | GI 338968872 NG_029246 Homo sapiens | TTGAAGCCCAGGCGG |
| NO: 2096 | RAR related orphan receptor A | AGACTTCAGATTCCT |
| | (RORA), RefSeqGene on chromosome 15 | TTGCATGTAGAAGTG |
| | | TTGGCCAAAAATAGC |
| SEQ ID | GI 959241467 NG_046314 Homo sapiens | AGAATAGAATAGTAC |
| NO: 2097 | signal transducer and | CTGGAGGTGGAAAGG |
| | activator of transcription 2 | GAGACGGGAACAAAT |
| | (STAT2), RefSeqGene on chromosome 12 | GTGGGGAAAGGAGGA |
| SEQ ID | GI 343432618 NG_029713 Homo sapiens | GAGGCGTGTCCCTGG |
| NO: 2098 | synoviolin 1 | CAGAGAGAGAATGTG |
| | (SYVN1), RefSeqGene on chromosome 11 | TTCAGTGACAACAGC |
| | | TCATATTTGTTGAGT |
| SEQ ID | GI 117606583 EF064766 Homo sapiens | CTCTCAGGATCAGCC |
| NO: 2099 | chemokine | TCATTGCTTTCCATA |
| | (C-C motif) ligand 19 | GTTCAGCGACACTAA |
| | (CCL19) gene, complete cds | GCTTGGGTCAGTTCC |
| SEQ ID | GI 66841738 DQ075211 Homo sapiens | CGTTGICACTGGAGT |
| NO: 2100 | c-src tyrosine kinase | GTGTGTGCCGTGACA |
| | (CSK) gene, complete cds | CCTTTTTTCATTTAA |
| | | TACTGCGTCTGIGGG |
| SEQ ID | GI 21914799 AY131997 Homo sapiens | TAGAGTTCCCATCAC |
| NO: 2101 | tumor necrosis factor receptor | TCCACACTGTCCAGT |
| | superfamily, member 1A | GACAACTCCCAACAT |
| | (TNFRSF1A) gene, complete cds | GGAAGATCTGCTAGT |
| SEQ ID | GI 966209932 NG_033919 Homo sapiens | TTACACAGCCAACTG |
| NO: 2102 | dedicator of cytokinesis 1 | GTCAGTGGTACAGCC |
| | (DOCK1), RefSeqGene on chromosome 10 | TGGATTTCATCTCTG |
| | | GGTGTTTATTCCTGG |
| SEQ ID | GI 224809291 NG_011495 Homo sapiens | CAATGCAGCCCCATA |
| NO: 2103 | interferon induced with helicase C domain 1 | TGATTCAGCAGAAGT |
| | (IFIH1), RefSeqGene on chromosome 2 | GGGTAGCAGTTGGGT |
| | | TTGATTAGTCTTGGC |
| SEQ ID | GI 1028224162 NG_047195 Homo sapiens | CCAAGAAGCCTCCTG |
| NO: 2104 | transducin beta like 1 X-linked receptor 1 | GGCTAAGCCTGAATT |
| | (TBL1XR1), RefSeqGene on chromosome 3 | TTGTCTCTTGTCTAC |
| | | CCTGCACTACAATCA |
| SEQ ID | GI 384367978 NM_021797 Homo sapiens | ATGTCATGAACTACT |
| NO: 2105 | chitinase, acidic | GGAAGGACAATGGAG |
| | (CHIA), transcript variant 2, mRNA | CACCAGCTGAGAAGC |
| | | TCATCGTTGGATTCC |
| SEQ ID | GI 164692857 AK313058 Homo sapiens | CATCTCTGCAATAGT |
| NO: 2106 | cDNA, FU93535, Homo sapiens | CCTTGGAGGCACCAA |
| | opioid receptor, kappa 1 | AGTCAGGGAAGACGT |
| | (OPRK1), mRNA | CGATGTCATTGAGTG |
| SEQ ID | GI 187763976 EU518415 Homo sapiens | AATCATGCCAACATC |
| NO: 2107 | serum/glucocorticoid | CTGACCAAGCCCGAT |
| | regulated kinase 1 isoform 2 | CCAAGAACCTTCTGG |
| | (SGK1) mRNA, complete cds | ACTAATGATGATCCA |
| SEQ ID | GI 206597516 NM_001859 Homo sapiens | GCTATCCTGTTGGCC |
| NO: 2108 | solute carrier family 31 member 1 | TGTGTTGTGGTTTTG |
| | (SLC31A1), mRNA | AAGTTTGTACTTTCT |
| | | CTGTGGGTGCCAGTT |
| SEQ ID | GI 476704 L26336 Homo sapiens | CTGGCTCGACCGAAA |
| NO: 2109 | heat shock protein | CCAGATGGCAGAGAA |
| | (HSPA2) gene, complete cds | AGATGAGTATGAACA |
| | | CAAGCAGAAAGAGCT |
| SEQ ID | GI 209447058 NG_008718 Homo sapiens | TTGTATGTCATCCTT |
| NO: 2110 | apurinic/apyrimidinic | CAGGCCTCAACCAAC |
| | endodeoxyribonuclease 1 | TACTTGCCCTCTACC |
| | (APEX1), RefSeqGene on chromosome 14 | TGCTGAAGTTTCTGC |
| SEQ ID | GI 237874281 NG_012193 Homo sapiens | GGAGGCAGCCCACCA |
| NO: 2111 | cytochrome c oxidase subunit 6B1 | GTTATTTTTATGCCG |
| | (COX6B1), RefSeqGene on chromosome 19 | TTTAACCGTAGGAAG |
| | | TCACTTAGACACCCT |
| SEQ ID | GI 694879461 NG_031891 Homo sapiens | AACAGAGGGCGCATT |
| NO: 2112 | sodium voltage-gated | TGGAGAGGTAATGAG |
| | channel alpha subunit 10 | CTGGAGTTCATGTGT |
| | (SCN10A), RefSeqGene on chromosome 3 | TCACACTCATGGTAA |
| SEQ ID | GI 346644679 NG_029975 Homo sapiens | ATTCACTCGAAGAAA |
| NO: 2113 | transforming growth factor alpha | GCTTCCCAGAGCAGG |
| | (TGFA), RefSeqGene on chromosome 2 | TGTTAGGGTTAGACC |
| | | AGTTAGGGGGAGTAG |
| SEQ ID | GI 574289185 NG_033890 Homo sapiens | TGCCTCACATTCAGC |
| NO: 2114 | transient receptor potential | AGGTTCAAAACCAAA |
| | cation channel subfamily A member 1 | CTTCAATGCTTTCAC |
| | (TRPAl), RefSeqGene on [symbol] | GTTTCACCCTCTCCT |
| SEQ ID | GI 116734711 NM_000020 Homo sapiens | GGATGAACAGTCCTG |
| NO: 2115 | activin A receptor like type 1 | GTTCCGGGAGACTGA |
| | (ACVRL1), transcript variant 1, mRNA | GATCTATAACACAGT |
| | | GTTGCTCAGACACGA |
| SEQ ID | GI 294489317 NG_017064 Homo sapiens | ATTAGAGTTGGCCAG |
| NO: 2116 | acyl-CoA dehydrogenase family member 9 | TCACTACAACTTGCC |
| | (ACAD9), RefSeqGene on chromosome 3 | TCCCTTACGCTTTTT |
| | | CTCGAGTTGGTCACC |
| SEQ ID | GI 224451013 NG_009888 Homo sapiens | CCATGTCCTCAGGGT |
| NO: 2117 | acetyl-CoA acetyltransferase 1 | CTAGAGTGGGGGCAA |
| | (ACAT1), RefSeqGene on chromosome 11 | TTTCTTATAACCTCA |
| | | ACATTCAGGGTTGGG |
| SEQ ID | GI 198442852 NG_008474 Homo sapiens | ;TCTGGGCTGTAGTTT |
| NO: 2118 | aryl hydrocarbon receptor | CCCCATCCGTAAAAT |
| | interacting protein like 1 | GGGGATAATAGTGCC |
| | (AIPL1), RefSeqGene on chromosome 1 | TGCCTTGAATTGTCA |
| SEQ ID | GI 685156953 NG_034254 Homo sapiens | TACCATTAGGGTGAC |
| NO: 2119 | cyclin D2 | GCTCGTGTGCATCTT |
| | (CCND2), RefSeqGene on chromosome 12 | CTGTGGCTGATGGGT |
| | | AAATTAAACAGGACG |
| SEQ ID | GI 237858686 NG_012087 Homo sapiens | CTCACTGTCCCTTTC |
| NO: 2120 | interleukin 6 receptor | AGGACCAAAATACTG |
| | (IL6R), RefSeqGene on chromosome 1 | GGTGTATCCATCTGC |
| | | AGGTACCAATGGCTT |
| SEQ ID | GI 1024846802 NG_046996 Homo sapiens | GCATTAAAGCCAAAG |
| NO: 2121 | mediator complex subunit 1 | TGACTTTGCAGAAAC |
| | (MED1), RefSeqGene on chromosome 17 | CTGGGGAAAGTAGTG |
| | | GAGAAGGGCTTAGGC |
| SEQ ID | GI 254039688 NG_012344 Homo sapiens | TCTCAGAAACCACCC |
| NO: 2122 | NFKB inhibitor like 1 | CCATCCCACAGAAAT |
| | (NFKBIL1), RefSeqGene on chromosome 6 | ATCCCAACACCAAAG |
| | | AGATCAACACAGTCC |
| SEQ ID | GI 237858596 NG_012143 Homo sapiens | CAACTCAGGCACACC |
| NO: 2123 | nuclear receptor | TACACAGACGCATAA |
| | subfamily 0 group B member 2 | AACAGACTCTCCAAC |
| | (NROB2), RefSeqGene on chromosome 1 | CACCCACTTATAGCC |
| SEQ ID | GI 345110626 NM_002001 Homo sapiens | GAGTCCATGAAGAAG |
| NO: 2124 | Fc fragment of IgE receptor 1a | ATGGCTCCTGCCATG |
| | (FCER1A), mRNA | GAATCCCCTACTCTA |
| | | CTGTGTGTAGCCTTA |
| SEQ ID | GI 52694751 NM_001005340 Homo sapiens | TACACACTCATGAAC |
| NO: 2125 | glycoprotein nmb | TCCTGATGGAACAAT |
| | (GPNMB), transcript variant 1, mRNA | AACAGGCCCAAGCCT |
| | | GTGGTATGATGTGCA |
| SEQ ID | GI 57165045 AY864066 Homo sapiens | ACTTCCGCCCACCCA |
| NO: 2126 | cyclin B2 | CTACAAACGTCGAGA |
| | (CCNB2) gene, complete cds | GAAATTCGTAGATGA |
| | | GGCCTTGTCAAAATT |
| SEQ ID | GI 32891808 AY341028 Homo sapiens | CTCACCAAGTTTTTC |
| NO: 2127 | microsomal glutathione S-transferase 2 | ATGAGGACGATCCGT |
| | (MGST2) gene, complete cds | CAGCATCTGTTCTGG |
| | | TCTCTGGTTTCCCAT |
| SEQ ID | GI 212549702 NG_008951 Homo sapiens | CAGGGCTCTCCAGAA |
| NO: 2128 | ATR serine/threonine kinase | CACACAAATGGAAAC |
| | (ATR), RefSeqGene on chromosome 3 | TTGCAGGCATTTTTA |
| | | ACAGGCCCCAAATTG |
| SEQ ID | GI 221139813 NG_009361 Homo sapiens | GAAATAGCCACCATG |
| NO: 2129 | BCL2, apoptosis regulator | TAGTGGGTTCTCACC |
| | (BCL2), RefSeqGene on chromosome 18 | ACATGACAGACACGG |
| | | TGCTGCTATAAGCTT |
| SEQ ID | GI 281182565 NG_016196 Homo sapiens | GAAACCAGAGAACTC |
| NO: 2130 | endothelin 1 | TAGGTGGAATGTGGT |
| | (EDN1), RefSeqGene on chromosome 6 | AACTTTTGCCAGGGC |
| | | AGAGGGAACACCTAC |
| SEQ ID | GI 257467496 NG_012839 Homo sapiens | CAGAGTCTTCCTGAT |
| NO: 2131 | glutamate metabotropic receptor 1 | GAGGCATTTCCTGTT |
| | (GRM1), RefSeqGene on chromosome 6 | CGCCTGAGAGCAATT |
| | | CCTCACAGAAGATGG |
| SEQ ID | GI 270309111 NG_016167 Homo sapiens | GAAACAGTATGTGGG |
| NO: 2132 | glycogen synthase 2 | GTCAGACTGCATGGA |
| | (GYS2), RefSeqGene on chromosome 12 | TTCTGCTTCGACTTA |
| | | CCAGCTGTGTGTTCT |
| SEQ ID | GI 187960074 NG_007666 Homo sapiens | AGAGCTCTGGACAAG |
| NO: 2133 | HRas proto-oncogene, GTPase | CAGGCAGATCATAAG |
| | (HRAS), RefSeqGene on chromosome 11 | GACAGAGAGCTTACT |
| | | GTGCTTCTACCAACT |
| SEQ ID | GI 335892866 NG_029106 Homo sapiens | CTGCTTGGGCAAAGG |
| NO: 2134 | interferon regulatory factor 7 | GGAACATTCTCTGGG |
| | (IRF7), RefSeqGene on chromosome 11 | TCAGATTTGTGGTTT |
| | | TAGAAGCTGTTCTGG |
| SEQ ID | GI 212549771 NG_008990 Homo sapiens | GGCTGAGGTTTTTAA |
| NO: 2135 | TNF superfamily member 11 | GGGGATTAGGGAGAG |
| | (TNFSF11), RefSeqGene on chromosome 13 | CGAGGGGTTAGAAAA |
| | | TTGGGGTCATTGAGT |
| SEQ ID | GI 1013417860 NG_046847 Homo sapiens | TGGAACTGTGGTTGC |
| NO: 2136 | ubiquitin specific peptidase 7 | CGTTGAAAGACCCAG |
| | (USP7), RefSeqGene on chromosome 16 | CATGTAATATCTTTC |
| | | TGATGGCAAAGTGCG |
| SEQ ID | GI 49574531 NM_019884 Homo sapiens | CGAGAGCTGCAGATC |
| NO: 2137 | glycogen synthase kinase 3 alpha | ATGCGTAAGCTGGAC |
| | (GSK3A), mRNA | CACTGCAATATTGTG |
| | | AGGCTGAGATACTTT |
| SEQ ID | GI 18141132 AF424815 Homo sapiens | ACCCATGTTACTGGG |
| NO: 2138 | SH2 domain containing SOCS box | GAGTGATGGATAAAT |
| | protein SOCS4 mRNA, complete cds | ACGCAGCCGAAGCAC |
| | | TACTGGAAGGAAAAC |
| SEQ ID | GI 345525588 NG_029933 Homo sapiens | AGGAGAGATGTGTGG |
| NO: 2139 | interleukin 16 | GATGCTCTTTGCGGG |
| | (IL16), RefSeqGene on chromosome 15 | GCAGATGGATTAAAT |
| | | GCCACCTTATGATTA |
| SEQ ID | GI 405778361 NG_032938 Homo sapiens | GTGTACACTGCCAGG |
| NO: 2140 | semaphorin 3B | GAGGAGCCACTGTTA |
| | (SEMA3B), RefSeqGene on chromosome 3 | TTCCGACTTTACACA |
| | | TGAGCAAACTGAGTT |
| SEQ ID | GI 594591639 NG_034041 Homo sapiens | AGCAACCACAGTGAT |
| NO: 2141 | sorbin and SH3 domain containing 1 | CATCTTAACGTTGGT |
| | (SORBS1), RefSeqGene on chromosome 10 | CCAAAACCCTCTAGT |
| | | GGCTTCTCATCTCGC |
| SEQ ID | GI 388490278 NG_032761 Homo sapiens | ATGATCTTCTTCTGC |
| NO: 2142 | TNF alpha induced protein 3 | CAGCTCAACTGAAAC |
| | (TNFAIP3), RefSeqGene on chromosome 6 | CCAGGAGGTTTCCTG |
| | | AAGGTGAAACCAAGA |
| SEQ ID | GI 343887349 NG_029780 Homo sapiens | TAAAGTCTCATGCCT |
| NO: 2143 | X-ray repair cross complementing 5 | CCAGGCGTTTGTGCA |
| | (XRCC5), RefSeqGene on chromosome 2 | TCCCATTTTCTCTGC |
| | | AGAGTAGAATCTCCC |
| SEQ ID | GI 22035651 NM_014349 Homo sapiens | CTCAGAAAAGAAACG |
| NO: 2144 | apolipoprotein L3 | CTTTACTGAAGAGGC |
| | (APOL3), transcript variant | CACCAAATACTTCCG |
| | alpha/a, mRNA | GGAGAGAGTCAGCCC |
| SEQ ID | GI 1010228574 NM_001321573 Homo sapiens | GAGCTTATGATTTTC |
| NO: 2145 | calcium/calmodulin dependent | CATCACCAGAATGGG |
| | protein kinase II delta | ACACGGTGACTCCTG |
| | (CAMK2D), transcript variant 14, [symbol] | AAGCCAAAGACCTCA |
| SEQ ID | GI 296010927 NM_172213 Homo sapiens | CCCTGCATACATAAA |
| NO: 2146 | CD8b molecule | GGTGCAAACCAACAA |
| | (CD8B), transcript variant 2, mRNA | GATGGTGATGCTGTC |
| | | CTGCGAGGCTAAAAT |
| SEQ ID | GI 387598072 NM_005277 Homo sapiens | TGTGGCAGATGTGTG |
| NO: 2147 | glycoprotein M6A | AGCGCTTGGTTCATT |
| | (GPM6A), transcript variant 1, mRNA | ATGCTGACATATCTT |
| | | TTCATGTTGGCCTGG |
| SEQ ID | GI 544709854 NM_001039845 Homo sapiens | TGCTCATGAGTCTGT |
| NO: 2148 | malate dehydrogenase 1B | CAGAGTCATCGTGGG |
| | (MDH1B), transcript variant 1, mRNA | AGGGAGAACCTTTGT |
| | | AAACCTGAAGACAGT |
| SEQ ID | GI 899148489 NM_001135690 Homo sapiens | CCTCCCTCCATCCTC |
| NO: 2149 | proenkephalin | CAGTGGGAAACTGTT |
| | (PENK), mRNA | GATGGTGTTTTATTG |
| | | TCATGTGTTGCTTGC |
| SEQ ID | GI 315360639 NM_012474 Homo sapiens | CGTTCGTCAAGCCTG |
| NO: 2150 | uridine-cytidine kinase 2 | CCTTTGAGGAATTCT |
| | (UCK2), mRNA | GCTTGCCAACAAAGA |
| | | AGTATGCTGATGTGA |
| SEQ ID | GI 1147489694 NG_016780 Homo sapiens | TATCTTGTTTTATAG |
| NO: 2151 | acyl-CoA thioesterase 9 | GCTTTGTGCCTTGGG |
| | (ACOT9), RefSeqGene on chromosome X | CAAAGGGCAGCTTAC |
| | | TCCTGGAAGAGGACT |
| SEQ ID | GI 343790959 NG_029764 Homo sapiens | CAGTCTGACAACATT |
| NO: 2152 | AKT serine/threonine kinase 3 | CCCTCTCTCTCTTCA |
| | (AKT3), RefSeqGene on chromosome 1 | TTCTCTGCATTGGCA |
| | | CCTTGACTTGCTGGT |
| SEQ ID | GI 291219938 NG_016779 Homo sapiens | TGAACAAAATCTGGT |
| NO: 2153 | interleukin 2 | GCCAGAAAGAGCTTG |
| | (IL2), RefSeqGene on chromosome 4 | TGCCAGGGTGAATCC |
| | | AAGCCCAGAAAATAA |
| SEQ ID | GI 975830227 NG_021386 Homo sapiens | AGTTGCCATGACCAG |
| NO: 2154 | TGF-beta activated kinase | AGCTTACTGAGATTA |
| | 1/MAP3K7 binding protein 2 | TGTGGTGAGGTCACC |
| | (TAB2), RefSeqGene on chromosome | TGAGTCAGGAACAGA |
| SEQ ID | GI 397938 Z22576 H. sapiens | CTGTGGGCTTTTATT |
| NO: 2155 | CD69 gene | GCCATGAGCTAGAAG |
| | | CATCACAGGTTGACC |
| | | AATAACCATGCCCAA |
| SEQ ID | GI 189181756 NM_001710 Homo sapiens | TTCCTTTGTACTGGA |
| NO: 2156 | complement factor B | GGAGTGAGTCCCTAT |
| | (CFB), mRNA | GCTGACCCCAATACT |
| | | TGCAGAGGTGATTCT |
| SEQ ID | ref \| NM_033229.2\| Homo sapiens | GTCTGCAGGTGTGAG |
| NO: 2157 | tripartite motif containing 15 | ATGAAGACTTTTGTG |
| | (TRIM15), mRNA | AGTCCTGAGGCCATT |
| | | TCTCCTGACCTTGTC |
| SEQ ID | GI 212549766 NG_008985 Homo sapiens | TGCCTGCGTATGGCT |
| NO: 2158 | N-acylsphingosine amidohydrolase 1 | ATAAATACTAAGGGG |
| | (ASAH1), RefSeqGene on chromosome 8 | AGTGTCAGGAGATGA |
| | | GGCTGGGATGGTAAG |
| SEQ ID | GI 326537295 NG_028240 Homo sapiens | TGTCAGGAAGAGGTA |
| NO: 2159 | cell division cycle 6 | TCCAGGCCAGCTGGG |
| | (CDC6), RefSeqGene on chromosome 17 | AAGGACATGATGAGG |
| | | AAATTGGAAAAACAT |
| SEQ ID | GI 344313160 NG_029811 Homo sapiens | ACACATTACCATCCA |
| NO: 2160 | carbohydrate sulfotransferase 1 | GTGTTCACAACACCC |
| | (CHST1), RefSeqGene on chromosome 11 | CTACAGGGTGAGGAT |
| | | CAGTACCTCTGTCTT |
| SEQ ID | GI 325995139 NG_008854 Homo sapiens | TAAGTCACTCAGTCT |
| NO: 2161 | potassium voltage-gated | CCTTCATCCAGGGTT |
| | channel subfamily Q member 3 | GGGACAGAGTCAGAC |
| | (KCNQ3), RefSeqGene on chromos [symbol] | TGGCTGATGCATTAG |
| SEQ ID | GI 1028630729 NG_050573 Homo sapiens | GTTTTGGTATCAGTG |
| NO: 2162 | NLR family pyrin domain containing 6 | TCACAGGCCTCAGAG |
| | (NLRP6), RefSeqGene on chromosome 11 | AATGAGTTGGGAAGC |
| | | ATTCCCTTGAGAGCT |
| SEQ ID | GI 1099522105 NG_052009 Homo sapiens | GTCATTCATCCTGTC |
| NO: 2163 | salt inducible kinase 1 | AATCAAGCGAGTGGG |
| | (SIK1), RefSeqGene on chromosome 21 | AATGGGGTGTGCATT |
| | | CTCTATTACCCAGGT |
| SEQ ID | GI 294459987 NG_017046 Homo sapiens | AGCAGTTGCATCCTT |
| NO: 2164 | spleen associated tyrosine kinase | CATGGCCATGACTTT |
| | (SYK), RefSeqGene on chromosome 9 | TGCTCTCTCCTTCCT |
| | | TTCTGGGAGATGTAT |
| SEQ ID | GI 1024336762 NG_046896 Homo sapiens | CGCACTTAAGAGTCC |
| NO: 2165 | TNF receptor superfamily member 4 | TGGGATCTGAATGAG |
| | (TNFRSF4), RefSeqGene on chromosome 1 | AATGACCACAGCGAC |
| | | TTCATGTAACGTGGC |
| SEQ ID | GI 32140476 AY316305 Homo sapiens | TGGTGGGAAGATTCA |
| NO: 2166 | glutathione transferase zeta 1 | TGAGTGCTAATCACT |
| | (maleylacetoacetate isomerase) | GGCGAGTCTTTCCTG |
| | (GSTZ1) gene, complete cds | GGTTCCACTTTGAAA |
| SEQ ID | GI 379991110 NG_032143 Homo sapiens | GACAGGGACTGTGGC |
| NO: 2167 | aconitase 2 | TTATTTATTCCTCTC |
| | (ACO2), RefSeqGene on chromosome 22 | TAGCTCTGGGTTTGG |
| | | GACCAGGTCAGTACA |
| SEQ ID | GI 184160977 NG_007553 Homo sapiens | TTCCTTTATTCACTT |
| NO: 2168 | actin, alpha, cardiac muscle 1 | TGGCAGCAGGGGATC |
| | (ACTC1), RefSeqGene on chromosome 15 | CTTTTCTGGCCTGAA |
| | | GACCAGAGTCTGAAT |
| SEQ ID | GI 696221409 NG_041768 Homo sapiens | CATCTGCTTTCATGC |
| NO: 2169 | CD24 molecule | CCCTGAATTTCTTTG |
| | (CD24), RefSeqGene on chromosome 6 | AGACCCTTCAAGTGC |
| | | ATTCCGAGGTGGTTC |
| SEQ ID | GI 343098522 NG_029664 Homo sapiens | TAGCCACTTCTGACT |
| NO: 2170 | cathepsin E | CAAGCTGCCCTTCTA |
| | (CTSE), RefSeqGene on chromosome 1 | GGTTTAGACCACTCT |
| | | CCCCAGTGAAAGATC |
| SEQ ID | GI 342307067 NG_029605 Homo sapiens | CAATGGGATACAAAA |
| NO: 2171 | interleukin 15 | GTTGGCAACACATTC |
| | (IL15), RefSeqGene on chromosome 4 | TTCCCCTTTTCTCTG |
| | | CCTGGGTGTGACCTC |
| SEQ ID | GI 1028224165 NG_047209 Homo sapiens | GAACCTGTACAGGAG |
| NO: 2172 | interleukin 33 | CACAGGAAAGAGTCA |
| | (IL33), RefSeqGene on chromosome 9 | AGGAGGAAAATGCTG |
| | | GCTTTCACATACCCT |
| SEQ ID | GI 916539820 NG_042809 Homo sapiens | TGCTAGCATGTATCA |
| NO: 2173 | potassium voltage-gated | TGTGTGTGGTGTGCA |
| | channel subfamily J member 12 | TAGCACATAGCACTG |
| | (KCNJ12), RefSeqGene on chromos [symbol] | TATGCTGGACCTGTT |
| SEQ ID | GI 480355034 KC485577 Homo sapiens | CACCTGGAAAACAAG |
| NO: 2174 | bone morphogenenic protein-5 | CGTCCTCTGCACCTC |
| | (BMP5) mRNA, complete cds | TCTTTATGCTGGATC |
| | | TCTACAATGCCATGA |
| SEQ ID | GI 45643118 NM_006117 Homo sapiens | CTGATAGCACTTTTC |
| NO: 2175 | enoyl-CoA delta isomerase 2 | AGAAAGAAGTCTGGA |
| | (ECI2), transcript variant 1, mRNA | CCAGGCTGAAGGCAT |
| | | TTGCAAAGCTTCCCC |
| SEQ ID | GI 229608964 NM_014600 Homo sapiens | TATGCATATCTTGAC |
| NO: 2176 | EH domain containing 3 | ATTGCTCTGTAGGTG |
| | (EHD3), mRNA | AGAGAGGACCATGAC |
| | | GCCCATGTTTGCAGC |
| SEQ ID | GI 21397157 AF517525 Homo sapiens | GCCAATTAAAGCCAG |
| NO: 2177 | cyclin D3 | GCCCTGATTGCTTTA |
| | (CCND3) gene, complete cds | AGGAAGGGCTGATGT |
| | | ATTTCATCCCACCTT |
| SEQ ID | GI 1168024004 KY497357 Homo sapiens | CCATAAGTGGAGTCC |
| NO: 2178 | HLA-DRA | CTGTGCTAGGATTTT |
| | (HLA-DRA) gene, complete cds | TCATCATAGCTGTGC |
| | | TGATGAGCGCTCAGG |
| SEQ ID | GI 1025608661 NG_047049 Homo sapiens | ATCCTACACTGAAAG |
| NO: 2179 | fatty acid binding protein 3 | CTCTTGGGTCTGGCT |
| | (FABP3), RefSeqGene on chromosome 1 | GTTCTCTGTGAAATG |
| | | CTGTAGTCTCTCCCT |
| SEQ ID | GI 207440658 NG_008647 Homo sapiens | CCCCTGTTGCGAAGG |
| NO: 2180 | GTP cyclohydrolase 1 | AAGATCCCTTATCTC |
| | (GCH1), RefSeqGene on chromosome 14 | TTCATCTGTCACTAA |
| | | TGATGGTGGCTTGCG |
| SEQ ID | GI 628601932 NG_034128 Homo sapiens | TCCACAAAAATTGCT |
| NO: 2181 | guanylate cyclase 1 soluble subunit alpha | CTCCCACTTGGTCCT |
| | (GUCY1A3), RefSeqGene on chromosome 4 | GTCCTAAGGCCTAAA |
| | | TCTCCTTTGGGTTGA |
| SEQ ID | GI 342307067 NG_029605 Homo sapiens | GCCATGTGAGTTTGG |
| NO: 2182 | interleukin 15 | ACACTGGGGTCTTGG |
| | (IL15), RefSeqGene on chromosome 4 | TCATTATGACATGCC |
| | | TATGCTCTAGGTAGC |
| SEQ ID | GI 325995139 NG_008854 Homo sapiens | ACCAAGTCTAAGGCA |
| NO: 2183 | potassium voltage-gated | GGGAGGATAAGAAAC |
| | channel subfamily Q member 3 | ATTGTCCCATTGAAC |
| | (KCNQ3), RefSeqGene on chromos [symbol] | AGCTGAGGAAGCAGG |
| SEQ ID | GI 908212441 NG_042288 Homo sapiens | TTGGAAACTCAGGCA |
| NO: 2184 | methylsterol monooxygenase 1 | GGGTTTCAGTGTTGC |
| | (MSM01), RefSeqGene on chromosome 4 | AGTCTCGAGAATCCT |
| | | TCTTCAGGAAACCTC |
| SEQ ID | GI 344925888 NG_029692 Homo sapiens | AAGCAAAAAGCCACA |
| NO: 2185 | 2′-5′-oligoadenylate synthetase 2 | GGTCATTAGGAAGTG |
| | (OAS2), RefSeqGene on chromosome 12 | ATGCTCCAACTGGGC |
| | | ATGGAAAAGGAGTTT |
| SEQ ID | GI 224967082 NG_011581 Homo sapiens | GTGGTTGCTGTTAAC |
| NO: 2186 | thyroid peroxidase | ACTATTGCATGACTC |
| | (TPO), RefSeqGene on chromosome 2 | CTGACCATAGGCTCT |
| | | GCCACTGACACTTCA |
| SEQ ID | GI 874507494 NG_042223 Homo sapiens | TTGGCCTAAGAGGCT |
| NO: 2187 | tubulin beta 2A class Ila | ACTCACAGACATCTG |
| | (TUBB2A), RefSeqGene on chromosome 6 | GCTTTAGTTTCCACC |
| | | TCCGATACAGACCAA |
| SEQ ID | GI 1047225539 NM_001201 Homo sapiens | ATTGAACCTCGGAAT |
| NO: 2188 | bone morphogenetic protein 3 | TGCGCCAGGAGATAC |
| | (BMP3), mRNA | CTCAAGGTAGACTTT |
| | | GCAGATATTGGCTGG |
| SEQ ID | GI 1049017896 NM_002192 Homo sapiens | TGGAAGGAGGGCAGA |
| NO: 2189 | inhibin beta A subunit | AATGAATGAACTTAT |
| | (INHBA), mRNA | GGAGCAGACCTCGGA |
| | | GATCATCACGTTTGC |
| SEQ ID | GI 91984778 NM_002214 Homo sapiens | GACAACTGAGTCACG |
| NO: 2190 | integrin subunit beta 8 | CACAACTCAACATGG |
| | (ITGB8), mRNA | AGCCTAACTGATTAT |
| | | CAGCTCAGATCCCGC |
| SEQ ID | GI 1002635078 NM_006256 Homo sapiens | CCCATTTCCAATCAG |
| NO: 2191 | protein kinase N2 | TCATGGGACCAGAAG |
| | (PKN2), transcript variant 1, mRNA | TTTACACTGGAACTG |
| | | GACAGGTCACGTGAA |
| SEQ ID | GI 32891806 AY340485 Homo sapiens | AGGACCATGGATGGG |
| NO: 2192 | biliverdin reductase B | AGACAGAGACCAGTG |
| | (flavin reductase | CAATGCCTGCATTAA |
| | (NADPH)) | CCCTTTAGTTGTTAG |
| | (BLVRB) gene, complete cds | |
| SEQ ID | GI 52854078 AY755659 Homo sapiens | TGCTCTGTCTCTTAC |
| NO: 2193 | small proline-rich protein 1A | CCTGGGGAGTTCATG |
| | (SPRRIA) gene, complete cds | ATCTGTAGAGGAAAA |
| | | GTGGGCTTCCTCATC |
| SEQ ID | GI 224809231 NG_011436 Homo sapiens | TAAACACAATTCACC |
| NO: 2194 | alpha 2-HS glycoprotein | TCCTCACACACTAGA |
| | (AHSG), RefSeqGene on chromosome 3 | CAGGAAACCAACGCA |
| | | GCTTGAAGCCAGTGA |
| SEQ ID | GI 1028630729 NG_050573 Homo sapiens | AAAACTTCCTAGTAT |
| NO: 2195 | NLR family pyrin domain containing 6 | GGCCCTACAGCTCTG |
| | (NLRP6), RefSeqGene on chromosome 11 | GCCCAGCCCTTCTTC |
| | | CCATTTTACTTTCTG |
| SEQ ID | GI 401709985 NG_032898 Homo sapiens | AAAAGGGAGTTCCCA |
| NO: 2196 | plasminogen activator, urokinase receptor | ACCTGCAAACAAATC |
| | (PLAUR), RefSeqGene on chromosome 19 | TCCCTCAAGTCATTG |
| | | GCAAGCTCTACATGC |
| SEQ ID | GI 365192584 NG_031951 Homo sapiens | AACTCTCCTCATATT |
| NO: 2197 | prostate transmembrane | TAAGGTCTCTGCTGA |
| | protein, androgen induced 1 | AAAGCCACCTCCTCC |
| | (PMEPA1), RefSeqGene on chromoso [symbol] | GAAAGGCTGTCCCTA |
| SEQ ID | GI 384367978 NM_021797 Homo sapiens | ATGGTTTCTACTCCT |
| NO: 2198 | chitinase, acidic | GAGAACCGCCAGACT |
| | (CHIA), transcript variant 2, mRNA | TTCATCACCTCAGTC |
| | | ATCAAATTCCTGCGC |
| SEQ ID | GI 208022628 NM_004887 Homo sapiens | CCTGGAACGAGAAGC |
| NO: 2199 | C-X-C motif chemokine ligand 14 | GCAGGGTCTACGAAG |
| | (CXCL14), mRNA | AATAGGGTGAAAAAC |
| | | CTCAGAAGGGAAAAC |
| SEQ ID | GI 194733730 NM_002993 Homo sapiens | AGCTGCGTTGCACTT |
| NO: 2200 | C-X-C motif chemokine ligand 6 | GTTTACGCGTTACGC |
| | (CXCL6), mRNA | TGAGAGTAAACCCCA |
| | | AAACGATTGGTAAAC |
| SEQ ID | GI 399567818 NM_003299 Homo sapiens | CCTGCTGCATGTCAC |
| NO: 2201 | heat shock protein 90 beta family member 1 | AGACACCGGTGTAGG |
| | (HSP90B1), mRNA | AATGACCAGAGAAGA |
| | | GTTGGTTAAAAACCT |
| SEQ ID | GI 124028520 NM_001080547 Homo sapiens | CCCTATGACACGGAT |
| NO: 2202 | Spi-1 proto-oncogene | CTATACCAACGCCAA |
| | (SPIl), transcript variant 1, mRNA | ACGCACGAGTATTAC |
| | | CCCTATCTCAGCAGT |
| SEQ ID | GI 374252456 NM_001252406 Homo sapiens | GGGGTCCCTGTCCCC |
| NO: 2203 | zinc finger and BTB domain containing 7B | AGCTGCTCCTGGGGA |
| | (ZBTB7B), transcript variant 3, mRNA | GTAGAAGCAATAATG |
| | | TATTTCTAATTTGTG |
| SEQ ID | GI 23955951 AF549495 Homo sapiens | AGTGTATTTAGTCGA |
| NO: 2204 | cyclin G2 | GACCTCTCTGCTGAG |
| | (CCNG2) gene, complete cds | CTTGCAACCTGTTTA |
| | | TTCACATGGCCTGCC |
| SEQ ID | GI 262331531 NG_013343 Homo sapiens | CCATAAGGACAGCAT |
| NO: 2205 | endothelin receptor type A | GAACTGACCACCCTT |
| | (EDNRA), RefSeqGene on chromosome 4 | AGAAGCACTCCTCGG |
| | | TACTCCCATAATCCT |
| SEQ ID | GI 239787846 NG_012306 Homo sapiens | ATCTTGGTCAGCTGA |
| NO: 2206 | interferon regulatory factor 5 | TAGTATCCGGCTACA |
| | (IRF5), RefSeqGene on chromosome 7 | GATCTCAAACCCAGA |
| | | CCTCAAAGACCGCAT |
| SEQ ID | GI 334883167 NG_029059 Homo sapiens | ACAAGTCACAGTTTC |
| NO: 2207 | mitogen-activated protein kinase 9 | TCAAGTGAGTTTGCA |
| | (MAPK9), RefSeqGene on chromosome 5 | GGCCGAAGTCCCCAG |
| | | GCAAAAGTCAAATTC |
| SEQ ID | GI 300116221 NG_023260 Homo sapiens | ATGTGGTCCCCAGTC |
| NO: 2208 | oxoglutarate dehydrogenase | ATCTCTGCTTCTAGA |
| | (OGDH), RefSeqGene on chromosome 7 | ATGTATGGAGCTCTC |
| | | AGTCCTGCTGGAAAA |
| SEQ ID | GI 401709985 NG_032898 Homo sapiens | CTTAGAACACATCTT |
| NO: 2209 | plasminogen activator, urokinase receptor | CCCCAAGAGTTCACC |
| | (PLAUR), RefSeqGene on chromosome 19 | CTGCTGGCAACTTCT |
| | | CATCCATTAGGCCTC |
| SEQ ID | GI 975830227 NG_021386 Homo sapiens | TGATGGACAACTTCA |
| NO: 2210 | TGF-beta activated kinase | AGGTGGCCAGTCCAA |
| | 1/MAP3K7 binding protein 2 | TAGTGAACTATTTCA |
| | (TAB2), RefSeqGene on chromosome [symbol] | GCAGGAGCCACAGAC |
| SEQ ID | GI 666335577 NM_006494 Homo sapiens | ACAAGCGCATTCTGC |
| NO: 2211 | ETS2 repressor factor | ACAAGACCAAGGGGA |
| | (ERF), transcript variant 1, mRNA | AACGGTTCACCTACA |
| | | AGTTCAATTTCAACA |
| SEQ ID | GI 167466188 NM_014009 Homo sapiens | TTAGAAGAGAGAGGT |
| NO: 2212 | forkhead box P3 | CTGCGGCTTCCACAC |
| | (FOXP3), transcript variant 1, mRNA | CGTACAGCGTGGTTT |
| | | TTCTTCTCGGTATAA |
| SEQ ID | GI 167234418 NM_005119 Homo sapiens | GTTTCGTTCCATTTT |
| NO: 2213 | thyroid hormone receptor | CCAGCACATACAATC |
| | associated protein 3 | AGCTCAGTCTCAGCG |
| | (THRAP3), mRNA | TAGCCCCTCAGAACT |
| SEQ ID | GI 284520137 NG_016421 Homo sapiens | CAACCAGAAAAGCAT |
| NO: 2214 | adrenoceptor beta 2 | TGGCCTTCTCTGCTT |
| | (ADRB2), RefSeqGene on chromosome 5 | CCTGTCAACTCAATG |
| | | ATGGGATGTTTGGGT |
| SEQ ID | GI 300244503 NG_023318 Homo sapiens | TTGCCAAGTACTTGC |
| NO: 2215 | Fc fragment of IgG receptor lib | TGCTCCTTCCCATGG |
| | (FCGR2B), RefSeqGene on chromosome 1 | AATACCTTTTCCCTC |
| | | TCAGCCAAAATCCTA |
| SEQ ID | GI 336088631 NG_029118 Homo sapiens | TTCTTGCCCTGGGGA |
| NO: 2216 | RAR related orphan receptor C | GAAAAAAAACTCAAA |
| | (RORC), RefSeqGene on chromosome 1 | CTATCCATGCTTAGG |
| | | GCAGGGGAGAGGTAG |
| SEQ ID | GI 260898757 NM_021571 Homo sapiens | ATGGGTTTGCACTAA |
| NO: 2217 | caspase recruitment domain | GAGAGAAGATGGAAC |
| | family member 18 | TCTGGAGCACTTCAG |
| | (CARD18), mRNA | AGACTTCCCAGAGCT |
| SEQ ID | GI 221316601 NM_001143935 Homo sapiens | AAGATCAAAAACTTA |
| NO: 2218 | carnitine O-octanoyltransferase | GCCGGGTATGGTGGT |
| | (CROT), transcript variant 1, mRNA | GCATGTGTGCCTGTA |
| | | GTCCAAGCTACTTGA |
| SEQ ID | GI 544709854 NM_001039845 Homo sapiens | TTGGATCGTGGAGGA |
| NO: 2219 | malate dehydrogenase 1B | AAGGGTTTGCTTTTG |
| | (MDH1B), transcript variant 1, mRNA | GGAGGATATAATGAG |
| | | TTCCTGGAGCATGCT |
| SEQ ID | GI 45934429 AY585238 Homo sapiens | AGTCTTTAGGGATAG |
| NO: 2220 | creatine kinase, muscle | GCCTTTTGGAGAACT |
| | (CKM) gene, complete cds | CAGTCCCAGGAAGGA |
| | | ACAGGAAAGAGGTGT |
| SEQ ID | GI 336176054 NG_029124 Homo sapiens | GTCACACAAGAAGGG |
| NO: 2221 | caspase 1 | AGGAGAGAAAAGCCA |
| | (CASP1), RefSeqGene on chromosome 11 | TGGCCGGTGAGTCTT |
| | | TTACTGTTCCAAGAA |
| SEQ ID | GI 533112518 NG_007159 Homo sapiens | GAGAACACAGTTCAT |
| NO: 2222 | chloride voltage-gated channel 5 | CATTCCCATTCAGCC |
| | (CLCN5), RefSeqGene on chromosome X | TGCCTTCATGTGTTC |
| | | AAGGGGCTGGAAAAG |
| SEQ ID | GI 190358513 NG_008045 Homo sapiens | CTGCTGGACTGAAGT |
| NO: 2223 | dihydrolipoamide dehydrogenase | TTGGAGGTGGATCTG |
| | (DLD), RefSeqGene on chromosome 7 | AGAAGGGGAGTGACA |
| | | CTGAGTTGCTAGTAT |
| SEQ ID | GI 237858687 NG_012088 Homo sapiens | TTGGGGAAACTAAGG |
| NO: 2224 | interleukin 10 | CCCAGAGACCTAAGG |
| | (IL10), RefSeqGene on chromosome 1 | TGACTGCCTAAGTTA |
| | | GCAAGGAGAAGTCTT |
| SEQ ID | GI 1027860277 NG_047185 Homo sapiens | CATCCCTATGCTCAA |
| NO: 2225 | NLR family member XI | CAAAGTTGGGTCAAG |
| | (NLRX1), RefSeqGene on chromosome 11 | CAGGTTAAGACGGCA |
| | | CATAGGTCACTGTGA |
| SEQ ID | GI 343488502 NG_029727 Homo sapiens | ATCCCAATTCCTGTC |
| NO: 2226 | platelet derived growth factor subunit A | AGGGTACTGCAACAT |
| | (PDGFA), RefSeqGene on chromosome 7 | GGCCAATGTGGCAAA |
| | | TGTGATTCTGGAAAT |
| SEQ ID | GI 574289185 NG_033890 Homo sapiens | CTGGACGGGCAACTG |
| NO: 2227 | transient receptor potential | CAGTCCAGGGAAACC |
| | cation channel subfamily A member 1 | TACTCAAAAAGTATC |
| | (TRPAl), RefSeqGene on [symbol] | TGTATCATCTGGCAC |
| SEQ ID | GI 14423572 AF388025 Homo sapiens | CTGCACCAGGCTTGT |
| NO: 2228 | colony stimulating factor 3 | CATTTACCATCTTCC |
| | (granulocyte) | ATAAACTTGAGCCCA |
| | (CSF3) gene, complete cds | TCCATCAAGTTGCCC |
| SEQ ID | GI 117606505 EF064727 Homo sapiens | ATACCATGGAAGGAA |
| NO: 2229 | inducible T-cell co-stimulator | CAAGTGCAAAGAGCC |
| | (ICOS) gene, complete cds | TGTGGCAGGAAGGAA |
| | | TGAGGTAAGGTTAGG |
| SEQ ID | GI 189339218 NG_007955 Homo sapiens | CTTACCCAAACTCTT |
| NO: 2230 | cytochrome P450 family 17 | CACAGCTGGGTTTCC |
| | subfamily A member 1 | CACGCTCTTCTTCCA |
| | (CYP17A1), RefSeqGene | ACCAACTGCAAATTC |
| | on chromosome [symbol] | |
| SEQ ID | GI 343488501 NG_029726 Homo sapiens | TTCATCCAACAAATC |
| NO: 2231 | dynamin 1 | CACTGGGCCGGAAAA |
| | (DNM1), RefSeqGene on chromosome 9 | TTTAAAGCACCTACC |
| | | CTATGCCAAGCAGTG |
| SEQ ID | GI 300244503 NG_023318 Homo sapiens | GCTTTTGACCACACA |
| NO: 2232 | Fc fragment of IgG receptor lib | GACCATGATGGGGAA |
| | (FCGR2B), RefSeqGene on chromosome 1 | GTACTGCATCTGATC |
| | | AGCAAGAGAGACAGG |
| SEQ ID | GI 162329557 NG_007161 Homo sapiens | TGAAAAGGCCCCCAA |
| NO: 2233 | MYC proto-oncogene, bHLH transcription factor | GGTAGTTATCCTTAA |
| | (MYC), RefSeqGene on chromosome 8 | AAAAGCCACAGCATA |
| | | CATCCTGTCCGTCCA |
| SEQ ID | GI 343488502 NG_029727 Homo sapiens | GTCATTGGTAAAGCA |
| NO: 2234 | platelet derived growth factor subunit A | GCTAATGCAAACGTG |
| | (PDGFA), RefSeqGene on chromosome 7 | GCTATCACAAGGCAT |
| | | CGGAAAATGGGAGCT |
| SEQ ID | GI 86278451 DQ374395 Homo sapiens | CCTTCAGAGAGAGGT |
| NO: 2235 | UDP glucuronosyltransferase 1 | GACTGTCCAGGACCT |
| | family, polypeptide A1 | ATTGAGCTCTGCATC |
| | (UGT1A1) gene, complete cds | TGTCTGGCTGTTTAG |
| SEQ ID | GI 401709984 NG_032897 Homo sapiens | AAACTTGACAAGACC |
| NO: 2236 | platelet factor 4 | AGCTCCTGAGACATG |
| | (PF4), RefSeqGene on chromosome 4 | GCACAGTGTTGACAC |
| | | CATGCTTATACCTCC |
| SEQ ID | GI 206725542 NG_008631 Homo sapiens | CTTTTTTGTATCCCA |
| NO: 2237 | patatin like phospholipase | AGGTGGCCTGTGGAC |
| | domain containing 3 | ACCCACATGGACAAA |
| | (PNPLA3), RefSeqGene on chromosome 22 | TGTTTACACTGGGAC |
| SEQ ID | GI 1147544507 NG_028250 Homo sapiens | ACCTGAGGGCTCTGA |
| NO: 2238 | PPARG coactivator 1 alpha | ATGGTCTGGGGTTTT |
| | (PPARGCIA), RefSeqGene on chromosome 4 | AAAGTAGCTACCCTT |
| | | GCGTCACAAAATGAG |
| SEQ ID | GI 899148489 NM_001135690 Homo sapiens | CAAGAGATATGGGGG |
| NO: 2239 | proenkephalin | CTTCATGAGAGGCTT |
| | (PENK), mRNA | AAAGAGAAGCCCCCA |
| | | ACTGGAAGATGAAGC |
| SEQ ID | GI 110735399 NM_001031680 Homo sapiens | CATTTGTGGAGAGAA |
| NO: 2240 | runt related transcription factor 3 | CAGGAGGGCCAGATA |
| | (RUNX3), transcript variant 1, mRNA | GATAGGTCCTAGCAG |
| | | AAGGCATTGAGGTGA |
| SEQ ID | GI 51988875 AY744675 Homo sapiens | ATCACTGGGCCATAT |
| NO: 2241 | matrix metalloproteinase 10 | ACTCAGACACTAAAA |
| | (stromelysin 2) | GACATCAGGAGAGGC |
| | (MMP10) gene, complete cds | CAGCTGAGGTCCAAA |
| SEQ ID | GI 338827643 NG_029228 Homo sapiens | ATCCATCTCTCCTAC |
| NO: 2242 | OS9, endoplasmic reticulum lectin | CAGAGTGTCCACCTT |
| | (OS9), RefSeqGene on chromosome 12 | AGGATGGTGCTCTCT |
| | | GCAGGCGAATATTTG |
| SEQ ID | GI 313747578 NM_015162 Homo sapiens | AAGTCTGTTAGATCT |
| NO: 2243 | acyl-CoA synthetase bubblegum family member 1 | CCAGGTCAGGGCACA |
| | (ACSBG1), transcript variant 1, mRNA | GCACTGGCTCTACAT |
| | | TTACCAGGTCTTACG |
| SEQ ID | GI 1084352529 NM_000755 Homo sapiens | CTCAGCTTGGTTCCT |
| NO: 2244 | carnitine O-acetyltransferase | GCCCCCGCCCTGCTG |
| | (CRAT), transcript variant 1, mRNA | TATGATATTAATGTG |
| | | GAAGGTCATCAATAA |
| SEQ ID | GI 544709854 NM_001039845 Homo sapiens | TCCATCAGCCATACC |
| NO: 2245 | malate dehydrogenase 1B | TAAGACCTGCACCTA |
| | (MDH1B), transcript variant 1, mRNA | AGTGGCCATGTTTGC |
| | | TCAAATGTCCTCATC |
| SEQ ID | GI 594140701 NM_002661 Homo sapiens | CGAAATGCTGGGCTT |
| NO: 2246 | phospholipase C gamma 2 | AGTATGCATGTACTG |
| | (PLCG2), mRNA | CTGAAAAGCAGGGCA |
| | | GAACAAATCAGGCTC |
| SEQ ID | GI 1036031503 AHO11685 Homo sapiens | TTGCATTTCTGGACT |
| NO: 2247 | chromosome 1 AMPK beta-2 subunit | GAACCAGTCTTACCT |
| | (PRKAB2) gene, complete cds | GAGACTGGAAGGCTG |
| | | ATTTGCTTTGAGGCT |
| SEQ ID | GI 21914605 AY130302 Homo sapiens | ACGAGGCTCTGAGGG |
| NO: 2248 | truncated recombination activating gene 1 | AGCTGATGGATCTTT |
| | (RAG1) gene, complete cds | ACCTGAAGATGAAAC |
| | | CAGTATGGCGATCAT |
| SEQ ID | GI 389565477 NG_032771 Homo sapiens | GAGCATGGTTCAGCT |
| NO: 2249 | bone morphogenetic protein 7 | CAGAGCTGTCCTTAT |
| | (BMP7), RefSeqGene on chromosome 20 | TGAATCGTTGCCACA |
| | | GCTAAGAGGTCACTG |
| SEQ ID | GI 341865607 NG_029555 Homo sapiens | CAGAACCCTCTAACC |
| NO: 2250 | ETS proto-oncogene 1, transcription factor | TTGGGCAGCTCCTTA |
| | (ETS1), RefSeqGene on chromosome 11 | CCTCTTCCTGTCTGA |
| | | GCCATGTTTGATTTG |
| SEQ ID | GI 537544658 NG_033791 Homo sapiens | TCAGTCCTGCTCCTT |
| NO: 2251 | gremlin 1, DAN family BMP antagonist | CTGCAAGCCCAAGAA |
| | (GREM1), RefSeqGene on chromosome 15 | ATTCACTACCATGAT |
| | | GGTCACACTCAACTG |
| SEQ ID | GI 340007392 NG_029438 Homo sapiens | CCTGGGAACACTGAC |
| NO: 2252 | mitogen-activated protein kinase kinase 7 | CATATCTAGTCTCCC |
| | (MAP2K7), RefSeqGene on chromosome 19 | GGCTTTTTCCCCAAG |
| | | ATCCTGGTCTCTAAA |
| SEQ ID | GI 194018566 NG_008183 Homo sapiens | TCCTTCTACCTCCCC |
| NO: 2253 | pancreatic and duodenal homeobox 1 | TTCTCATCCCTCCTC |
| | (PDX1), RefSeqGene on chromosome 13 | TTCCTCTTCTCTAGC |
| | | TGCACACTTCACTAC |
| SEQ ID | GI 328751683 NG_028290 Homo sapiens | ATGAAGATACTGTGT |
| NO: 2254 | acyl-CoA synthetase long | GGCCCAAACGAATAC |
| | chain family member 6 | CAGTTTCCTTTAGCC |
| | (ACSL6), RefSeqGene on chromosome 5 | CCAGAGGCTGACAAG |
| SEQ ID | GI 237874281 NG_012193 Homo sapiens | TTCTTCATGGTGCCC |
| NO: 2255 | cytochrome c oxidase subunit 6B1 | CCAAAAGAATTGCCA |
| | (COX6B1), RefSeqGene on chromosome 19 | CACAATAACAGCCCA |
| | | GAATATCCCCAACAG |
| SEQ ID | GI 341823644 NG_029509 Homo sapiens | TGATCATGCCTGGGT |
| NO: 2256 | prostaglandin E receptor 3 | GGCTGTGCTCAAGAC |
| | (PTGER3), RefSeqGene on chromosome 1 | TTTCACTTTAAGGTC |
| | | TGAACCAGTAAGTGG |
| SEQ ID | GI 254826724 NG_012488 Homo sapiens | GAGAGAAACAGATGA |
| NO: 2257 | prostaglandin E synthase 2 | CCAGGAACCGCCTTG |
| | (PTGES2), RefSeqGene on chromosome 9 | GTTCACATAACTGAC |
| | | AAGTTCAGGGAACGG |
| SEQ ID | GI 237858776 NG_012127 Homo sapiens | GACCCAGCTTTAGAG |
| NO: 2258 | solute carrier family 2 member 4 | TCCAGGGAGAGCTGA |
| | (SLC2A4), RefSeqGene on chromosome 17 | CCGTCATAAGAACTG |
| | | AGAGGCCATAACATT |
| SEQ ID | GI 38016940 NM_014365 Homo sapiens | TGTCAGTTTATGATC |
| NO: 2259 | heat shock protein family B | TGACCTAGGTCCCCC |
| | (small) member 8 | CTTTCTTCTGTCCCC |
| | (HSPB8), mRNA | TGTGTTTAAGTCGGG |
| SEQ ID | GI 1019399995 LT222231 Homo sapiens | GTGGCTCAGGTGTTT |
| NO: 2260 | IncRNAfor PRTN3 gene | CTGAACAACTACGAC |
| | | GCGGAGAACAAACTG |
| | | AACGACATTCTCCTC |
| SEQ ID | GI 39725959 NM_175852 Homo sapiens | GTACAGATGAAGTCA |
| NO: 2261 | taxilin alpha | GTGGCTTGTCTGTGA |
| | (TXLNA), mRNA | GCTGAAGAGTCTTGA |
| | | GAGGGGCTGTCATCT |
| SEQ ID | GI 14278706 AF378903 Homo sapiens | AGCTTCTGGAATTGT |
| NO: 2262 | protein C | TGCTGTTGTGCCCTT |
| | (PROC) gene, complete cds | TCCATTCTTTTGTTA |
| | | TGATGCAGCTCCCCT |
| SEQ ID | GI 56157779 AY35b29 Homo sapiens | GCTTGCATAGTTCCT |
| NO: 2263 | superoxide dismutase 1, soluble | TCTTTTAAGCGCCTG |
| | (amyotrophic lateral sclerosis 1 | ACTTCGTTTTGTTTG |
| | (adult)) | GTGCCCGTTGTACCT |
| | (SUD1) gene, complete | |
| SEQ ID | GI 19909347 AF495471 Homo sapiens | GGTGCAAGTGGACCT |
| NO: 2264 | thrombomodulin | GTGTAGGACGTCCAG |
| | (THBD) gene, complete cds | TGAAACATTGTGTTG |
| | | GTGAATCAGCTAGAA |
| SEQ ID | GI 326806980 NG_028248 Homo sapiens | CTACATCACAGAACT |
| NO: 2265 | aryl hydrocarbon receptor | GTCAGATATGGTACC |
| | nuclear translocator | CACCTGTAGTGCCCT |
| | (ARNT), RefSeqGene on chromosome 1 | GGCTCGAAAACCAGA |
| SEQ ID | GI 345478675 NG_029883 Homo sapiens | TCGTTGTCATCTGGT |
| NO: 2266 | death associated protein kinase 1 | AGAGTCATGCCCATC |
| | (DAPK1), RefSeqGene on chromosome 9 | ACGTAACAGCTGCCA |
| | | ATGCATATTTCTGCA |
| SEQ ID | GI 306922359 NG_027701 Homo sapiens | TGCTTAGAGAGGTTA |
| NO: 2267 | retinoic acid receptor alpha | AGTGACTTGCGTAAG |
| | (RARA), RefSeqGene on chromosome 17 | GTCACCCAGAGTGGC |
| | | AGAGCTGAGATTTGA |
| SEQ ID | GI 594591639 NG_034041 Homo sapiens | GCCAGCAGTGGTCTT |
| NO: 2268 | sorbin and SH3 domain containing 1 | TATGCTGGCTAGTTT |
| | (SORBS1), RefSeqGene on chromosome 10 | TGAGAATGACAGTCT |
| | | GGAGACTGATCCAGC |
| SEQ ID | GI 189491779 NM_006507 Homo sapiens | TGAGACCTGGGTTGA |
| NO: 2269 | regenerating family member 1 beta | TGCAGATCTCTATTG |
| | (REGIB), mRNA | CCAGAACATGAATTC |
| | | AGGCAACCTGGTGTC |
| SEQ ID | GI 343790959 NG_029764 Homo sapiens | GGTGGGAAACTAGGG |
| NO: 2270 | AKT serine/threonine kinase 3 | AGTTGCTGGTATAAA |
| | (AKT3), RefSeqGene on chromosome 1 | GCCTGGAGTCCAAAT |
| | | GTCAGAGAGCCTAGA |
| SEQ ID | GI 212549700 NG_008949 Homo sapiens | ATTAGCTGGCATAGC |
| NO: 2271 | apolipoprotein C3 | AGAGGTGTTCATAAA |
| | (APOC3), RefSeqGene on chromosome 11 | CATTCTTAGTCCCCA |
| | | GAACCGGCTTTGGGG |
| SEQ ID | GI 298104098 NG_021428 Homo sapiens | CACTCATGCCCACCC |
| NO: 2272 | C-C motif chemokine receptor 2 | AGGGTAAGTCTAATC |
| | (CCR2), RefSeqGene on chromosome 3 | ATGTTCACCAATCAG |
| | | AACCTCTGCCACATC |
| SEQ ID | GI 237820700 NG_012083 Homo sapiens | TTTGCAATCAGGTGA |
| NO: 2273 | intercellular adhesion molecule 1 | GACCTCCTCTGATGG |
| | (ICAM1), RefSeqGene on chromosome 19 | GGAAAATGACACGGT |
| | | GAGTGACAAAGGATG |
| SEQ ID | GI 570700862 NG_033874 Homo sapiens | AAGGAGAAACGCTAC |
| NO: 2274 | nuclear factor kappa B subunit 2 | TGACCAGAGAGAGGT |
| | (NFKB2), RefSeqGene on chromosome 10 | CCAGTCATGTACTCC |
| | | GTGCTTGGTGAATTT |
| SEQ ID | GI 291219867 NG_016781 Homo sapiens | GCAATCCCACTAGGA |
| NO: 2275 | pyruvate dehydrogenase alpha 1 | CACAACACAATGGGT |
| | (PDHA1), RefSeqGene on chromosome X | TCCTGAGCCCTTTGA |
| | | TCCTCTGAATAGAGC |
| SEQ ID | GI 654825134 NG_008781 Homo sapiens | CAGGAGCTGAGGATA |
| NO: 2276 | poliovirus receptor | AAGGGCCCGATCTTT |
| | (PVR), RefSeqGene on chromosome 19 | CTTTGGGCAAGGTTA |
| | | AGCCTTTACTGCATA |
| SEQ ID | GI 1027250682 NG_047124 Homo sapiens | AAGGCAAAAAGACAC |
| NO: 2277 | solute carrier family 27 member 5 | CATAGGTTACCCAGC |
| | (SLC27A5), RefSeqGene on chromosome 19 | ACCCTGTGTTCTCAC |
| | | TTTTGAGAAGGGACA |
| SEQ ID | GI 387849236 NM_002613 Homo sapiens | CAGGCTTCCTCCTCC |
| NO: 2278 | 3-phosphoinositide dependent | TCATCAGCTATTTTA |
| | protein kinase 1 | CCCATCTCAGAACGT |
| | (PDPK1), transcript variant 1, mRNA | CCTGTGTCTCCATGT |
| SEQ ID | GI 1212624021 NM_017523 Homo sapiens | AGGTTCCAGTTTTGT |
| NO: 2279 | XIAP associated factor 1 | CTGAAAATTCTGATT |
| | (XAF1), transcript variant 1, mRNA | AAGCCTCTGGGCCCT |
| | | ACAGCCTGGAGAACC |
| SEQ ID | GI 1657840 U73304 Human CB1 | ATACTTTGCATTGTC |
| NO: 2280 | cannabinoid receptor | TGTCTGCACACCTTG |
| | (CNR1) gene, complete cds | AATCTGCCTGCTGGC |
| | | TCCCTTACTTTACCT |
| SEQ ID | GI 14423570 AF387519 Homo sapiens | GAGCACATAGAAAAA |
| NO: 2281 | interleukin 22 | ATTCCCAGCAAGCCT |
| | (IL22) gene, complete cds | TTGTCTTTTCCTGGC |
| | | AGTCAGCCCCTCTTT |
| SEQ ID | GI 32891808 AY341028 Homo sapiens | AGCATAGGCCCCCAT |
| NO: 2282 | microsomal glutathione S-transferase 2 | AATGGGATCTGTGCC |
| | (MGST2) gene, complete cds | CTTATAAGAAGTGAC |
| | | CAGGAAGCTTACTCT |
| SEQ ID | GI 1154067877 NG_052975 Homo sapiens | TGAAGAAGGAGGCTG |
| NO: 2283 | C-X-C motif chemokine receptor 2 | ACTGGGAAATGTCAA |
| | (CXCR2), RefSeqGene on chromosome 2 | ATCTGGGCTCCCCTC |
| | | TCTACATCCTTTGTA |
| SEQ ID | GI 1025608661 NG_047049 Homo sapiens | GGCCTGGAGTTTAGA |
| NO: 2284 | fatty acid binding protein 3 | ATCAGGGCTCTTTCC |
| | (FABP3), RefSeqGene on chromosome 1 | AAAGACACTGCATCT |
| | | GAAGATGTCAGAGGC |
| SEQ ID | GI 214010161 NG_009030 Homo sapiens | GAGTCCAGGACTGAG |
| NO: 2285 | GLI family zinc finger 2 | GTTCGGAAATCCTGG |
| | (GLI2), RefSeqGene on chromosome 2 | GTTTTTTCACCCCAT |
| | | TACTGGATGACTTGG |
| SEQ ID | GI 323668281 NG_023034 Homo sapiens | TTCCCAAGAAACTGT |
| NO: 2286 | vascular cell adhesion molecule 1 | GCTTGGCCGGCTGCT |
| | (VCAM1), RefSeqGene on chromosome 1 | TTCAATTATGCTGGT |
| | | TGTATGTGCATAGTT |
| SEQ ID | GI 7407497 AF243044 Homo sapiens | GGCATGAGATGCCCT |
| NO: 2287 | ECSIT | TTGAGTGTACAGCAA |
| | (ECSIT) mRNA, complete cds | ATCAATGTTTTCCTG |
| | | CTTGGGGCTCTCTTC |
| SEQ ID | GI 20530138 AF261937 Homo sapiens | AGGCTGCTCACTTAG |
| NO: 2288 | NFKBIE protein | GAAACCATATGCCAG |
| | (NFKBIE) gene, complete cds | ACACTGAGCTAGAAA |
| | | CTGAGGAGACAGACC |
| SEQ ID | GI 237649059 NG_012020 Homo sapiens | GAGGGAAATTAAGGT |
| NO: 2289 | adrenoceptor alpha 2A | GAAATGGCTCCCACA |
| | (ADRA2A), RefSeqGene on chromosome 10 | GGGATCTTGGCTTAA |
| | | AGAGCTAACCCCACA |
| SEQ ID | GI 577019604 NG_033916 Homo sapiens | CCCTGGCAAGGAAAT |
| NO: 2290 | growth differentiation factor 2 | GAGAAAGTATCCTAA |
| | (GDF2), RefSeqGene on chromosome 10 | TTAGGGGGCAATAAC |
| | | CCCCTGGAGAAGCAC |
| SEQ ID | GI 341823675 NG_029547 Homo sapiens | GTGTAGGATGATCTC |
| NO: 2291 | interleukin 5 receptor subunit alpha | AGGTGACAGACAGCA |
| | (IL5RA), RefSeqGene on chromosome 3 | GCAATTTATCACACC |
| | | CAGAAACGCATCCCC |
| SEQ ID | GI 237858721 NG_012100 Homo sapiens | GCTGCAAGGGGTGAG |
| NO: 2292 | matrix metallopeptidase 3 | GACACCAGCATGAAC |
| | (MMP3), RefSeqGene on chromosome 11 | CTTGTTCAGGTAATT |
| | | AACACTAACTGACCT |
| SEQ ID | GI 608785172 KJ534783 Homo sapiens | TCCTTAAGGAGGAAA |
| NO: 2293 | clone BZRAPl_iso-A_adult-A09 | ACCAGATGCTGAGGA |
| | benzodiazapine receptor peripheral- | AGAGCAGCTTCCCTG |
| | associated protein l isofo [symbol] | AGACAGAAGAGAAGG |
| SEQ ID | GI 153082751 NM_002199 Homo sapiens | CTTCTCTTTAACTCA |
| NO: 2294 | interferon regulatory factor 2 | GGACTCCAGCCCATT |
| | (IRF2), mRNA | GGTAGACGTGTGTTT |
| | | CTAGAGCCTGCTGGA |
| SEQ ID | GI 187423911 NM_003764 Homo sapiens | TTTTTCTACTAGCTA |
| NO: 2295 | syntaxin 11 | CTGAGGCTGCCACAT |
| | (STX11), mRNA | CTGGATGGAACTGAG |
| | | TGGAGGGGGAAAAGA |
| SEQ ID | GI 48772828 AY618665 Homo sapiens | CTTCATCTGTCCTGA |
| NO: 2296 | VIMP mRNA, complete cds | AACGGAAATCGGACA |
| | | GAAAGCCTTTGCGGG |
| | | GAGGAGGTTATAACC |
| SEQ ID | GI 170014713 NG_007461 Homo sapiens | TCTAGAAGCCACTCA |
| NO: 2297 | transforming growth factor beta receptor 1 | AATTCTTTGGCTCGT |
| | (TGFBR1), RefSeqGene on chromosome 9 | GGTTCCCTCCTCCAT |
| | | CATCAAAACCAGCAG |
| SEQ ID | GI 224809355 NG_011515 Homo sapiens | GACCCAGTGAAGTTT |
| NO: 2298 | uncoupling protein 3 | AAGGGCAGGACACCT |
| | (UCP3), RefSeqGene on chromosome 11 | CTTAAGTTGGGGCTC |
| | | TTCACTCACTTGTTG |
| SEQ ID | GI 193211454 NM_020651 Homo sapiens | GGTCCCAGATCCCAC |
| NO: 2299 | pel lino E3 ubiquitin protein ligase 1 | TTCCTCATGGTACTC |
| | (PELIl), mRNA | ATACTTTTCATGCAG |
| | | CCTGTCCCTTTTGTG |
| SEQ ID | GI 21634442 AF274025 Homo sapiens | TATCAGACTGTTCCC |
| NO: 2300 | GTP-binding protein Rab25 | TGTTCACAGCACCCT |
| | mRNA, complete cds | CAGGGTCTTAAGGTC |
| | | TTCATGCCCTATCAC |
| SEQ ID | GI 1036032362 AH002667 Homo sapiens | GTTTCAAGGCTTAGT |
| NO: 2301 | cytochrome P450 | GTGTGTTGTGGTGTG |
| | (CYP1A2) gene, complete cds | CGTCAGTAGCTCTTG |
| | | GTGCTTAGTCTGGAA |
| SEQ ID | GI 50234986 AY675319 Homo sapiens | ATGCAGTCTAGGTAC |
| NO: 2302 | transforming growth factor, beta receptor II | CACAAGTAGTGTTCT |
| | (70/80 kDa) | CTTGTGCAAGCACAG |
| | (TGFBR2) gene, complete cds | CTTCCAGCTGTGTTG |
| SEQ ID | GI 353703739 NG_030372 Homo sapiens | CTATTTGAAGACAGA |
| NO: 2303 | CD83 molecule | CACAGGGATTGGAGT |
| | (CD83), RefSeqGene on chromosome 6 | GATGTGTCTACAAGC |
| | | CAAGCAACGCCGAGG |
| SEQ ID | GI 212549732 NG_008981 Homo sapiens | ACTTGAGAGGCAGAC |
| NO: 2304 | growth differentiation factor 6 | ATACAGAGCAAGACA |
| | (GDF6), RefSeqGene on chromosome 8 | CAGGGCAGGCCAAAA |
| | | AAGACCACTTACATC |
| SEQ ID | GI 237858686 NG_012087 Homo sapiens | TATATCCAAGAAAGA |
| NO: 2305 | interleukin 6 receptor | AACCTGCTGCTCCCC |
| | (IL6R), RefSeqGene on chromosome 1 | TCTCTGCTGAGGGAA |
| | | GAGCAAGAGGAACTT |
| SEQ ID | GI 240255451 NG_012330 Homo sapiens | GGCCTTGGGCAAGTT |
| NO: 2306 | MYB proto-oncogene, transcription factor | ACCTCCATTTTCTGT |
| | (MYB), RefSeqGene on chromosome 6 | CTCCATTCTCGAGCC |
| | | TGTCTAGAGAGAGAA |
| SEQ ID | GI 4826562 Y17151 Homo sapiens | ACTGGGGTGAAGTTT |
| NO: 2307 | mRNA for multidrug resistance protein 3 | CGTACTGGGATCATG |
| | (ABCC3) | GGTGTCATCTACAGG |
| | | AAGGCTCTGGTTATC |
| SEQ ID | GI 574584717 NM_001289177 Homo sapiens | TAGCTACCTCCCACT |
| NO: 2308 | acyl-CoA synthetase | CAGCCATATTGCAGC |
| | bubblegum family member 2 | ACAGATGATGGACAT |
| | (ACSBG2), transcript variant 1, mRNA | CTGGGTACCCATAAA |
| SEQ ID | GI 874507471 NM_005928 Homo sapiens | CTGTCCAGTTTGTGG |
| NO: 2309 | milkfat globule-EGF factor 8 protein | CATCCTACAAGGTTG |
| | (MFGE8), transcript variant 1, mRNA | CCTACAGTAATGACA |
| | | GTGCGAACTGGACTG |
| SEQ ID | GI 110735399 NM_001031680 Homo sapiens | CACTCTGCATACGCT |
| NO: 2310 | runt related transcription factor 3 | TCTGTTGAGAATTTG |
| | (RUNX3), transcript variant 1, mRNA | TGGCTAGACATTCCT |
| | | GTGGGACCGGGAATC |
| SEQ ID | GI 356460958 NG_030484 Homo sapiens | GCATTTTATATTTTC |
| NO: 2311 | adenosine kinase | CTAGGCTGAGGCTTC |
| | (ADK), RefSeqGene on chromosome 10 | CCAGGCTGTTGCTTT |
| | | TTTTCCCCCCACCTT |
| SEQ ID | GI 260763953 NG_013056 Homo sapiens | TTGCCAAGAGTCACA |
| NO: 2312 | chitinase 3 like 1 | CAGCAAGTTAACAGT |
| | (CHI3L1), RefSeqGene on chromosome 1 | GGATCTCCCAATTCC |
| | | CTGCCAATGCTCTAT |
| SEQ ID | GI 343403846 NG_029699 Homo sapiens | CAACTATAACTCCAG |
| NO: 2313 | protein kinase cAMP-activated | TGATGACTTGCATAC |
| | catalytic subunit alpha | AAGGCGTGGGCTCAG |
| | (PRKACA), RefSeqGene on chromoso [symbol] | AGGGTTTGGGGAAAA |
| SEQ ID | GI 300116282 NG_023271 Homo sapiens | CTGTTTCTGTTCTCT |
| NO: 2314 | regenerating family member 1 alpha | CCTGCTTAGCTCCAG |
| | (REGIA), RefSeqGene on chromosome 2 | GGATGGAACTGGGAC |
| | | TGGGATAGAGGAAAG |
| SEQ ID | GI 194294528 NG_008241 Homo sapiens | AGGAGCTGCAAGGAG |
| NO: 2315 | succinate-CoA ligase | AATGTTGTGGTTAGA |
| | ADP-forming beta subunit | TTGGAGTGAGGAAGG |
| | (SUCLA2), RefSeqGene on chromosome 13 | GGTAGGGTACTGAAA |
| SEQ ID | GI 164690417 AK312618 Homo sapiens | ACAGCTGGTCAAGAA |
| NO: 2316 | cDNA, FLI93000, Homo sapiens | CGATACCATAGCCTA |
| | RAB5A, member RAS oncogene family | GCACCAATGTACTAC |
| | (RAB5A), mRNA | AGAGGAGCACAAGCA |
| SEQ ID | GI 263191773 NG_015830 Homo sapiens | CCTCCCCAATTTTTG |
| NO: 2317 | insulin receptor substrate 1 | CAAAGTAGGAACTGG |
| | (IRS1), RefSeqGene on chromosome 2 | AGTTTTCTCGTTGCT |
| | | CCAAAGCTGTGAGGC |
| SEQ ID | GI 1039673930 NG_050913 Homo sapiens | CTGTACACCATCTCG |
| NO: 2318 | nuclear receptor subfamily 5 group A member 2 | TAATCGCGAAAGTTA |
| | (NR5A2), RefSeqGene on chromosome 1 | TCACCATGTTGGAAA |
| | | AGCCCACGCCCTTCT |
| SEQ ID | GI 211904121 NG_008930 Homo sapiens | AAGAAGCTGTGAGTG |
| NO: 2319 | cytochrome p450 oxidoreductase | CAGGTGTTAGAGACA |
| | (POR), RefSeqGene on chromosome 7 | GGCAGCATGAAGAGG |
| | | AGGGAATAGAGTCAG |
| SEQ ID | GI 1147592400 NG_029037 Homo sapiens | TTGAAAAATGTCAAC |
| NO: 2320 | reticulon 4 | TTGGGATGTGTCTTG |
| | (RTN4), RefSeqGene on chromosome 2 | GAGGAGGGACTGACA |
| | | AGGCCTGCTGATCTC |
| SEQ ID | GI 172072641 NM_005373 Homo sapiens | AGGACCCTTTCTCCA |
| NO: 2321 | MPL proto-oncogene, thrombopoietin receptor | CAGGCAGGCTCATTT |
| | (MPL), mRNA | CACTAAGCTCCTCCT |
| | | TTACTTTCTCTCTCC |
| SEQ ID | GI 156616272 NM_002664 Homo sapiens | AGAGGGGCTGTTAGC |
| NO: 2322 | pleckstrin | ATTGCGCAGCATCTT |
| | (PLEK), mRNA | CAGTTCTCCAGTAAA |
| | | TGATATTGCGTTCGT |
| SEQ ID | GI 269914182 NM_178859 Homo sapiens | AAGTGACAGCTGTTA |
| NO: 2323 | solute carrier family 51 beta subunit | CTGCCTGGGAGAGCT |
| | (SLC51B), mRNA | CAAGTGCAAAGACTA |
| | | TCCTGTTCTCCCATA |
| SEQ ID | GI 315434201 NM_020429 Homo sapiens | ATGCGACCGAAAGAC |
| NO: 2324 | SMAD specific E3 ubiquitin protein ligase 1 | TTGAAAAAACGGCTG |
| | (SMURF1), transcript variant 1, mRNA | ATGGTGAAATTCCGT |
| | | GGGGAAGAAGGTTTG |
| SEQ ID | GI 60391402 AB019488 Homo sapiens | CTGGAGACCCGGTGG |
| NO: 2325 | NTRK1 gene for TRKA, complete cds | AGAAGAAGGACGAAA |
| | | CACCTTTTGGGGTGA |
| | | GATAGGAAGTAGAAG |
| SEQ ID | GI 301500676 NG_023437 Homo sapiens | GTGATTCTCAGCTGA |
| NO: 2326 | apolipoprotein L4 | AATAGATTGGTTCCT |
| | (APOL4), RefSeqGene on chromosome 22 | CCACAAAGCATCGTG |
| | | GCACTTCCCTGGTGA |
| SEQ ID | GI 237858602 NG_012146 Homo sapiens | GAGAATGGAGTCTGA |
| NO: 2327 | period circadian clock 2 | GGTGAGCACTTTACT |
| | (PER2), RefSeqGene on chromosome 2 | GGGAATTGCAGTCCC |
| | | AGGGTAGCAGTAGAA |
| SEQ ID | GI 597709817 NM_032965 Homo sapiens | AGCATTCTGACCTCT |
| NO: 2328 | C-C motif chemokine ligand 15 | GCCCTGGGACTTCCT |
| | (CCL15), mRNA | GGATCCTCCTCTTCT |
| | | TATAAATACAAGGGC |
| SEQ ID | GI 1041817991 NM_000569 Homo sapiens | CATCATCCTCAGGCC |
| NO: 2329 | Fc fragment of IgG receptor Illa | TCTCTACAAGCAGCA |
| | (FCGR3A), transcript variant 1, mRNA | GGAAACATAGAACTC |
| | | AGAGCCAGATCCCTT |
| SEQ ID | GI 156105692 NM_001037335 Homo sapiens | GCGGCCCACATCATG |
| NO: 2330 | helicase with zinc finger 2 | GTGAAGGAGTACATG |
| | (HELZ2), transcript variant 1, mRNA | ATTCAGTTTAATAGG |
| | | CTCGTGGCTGAGTTC |
| SEQ ID | GI 30526191 AY275469 Homo sapiens | GACACCATCTTCCAC |
| NO: 2331 | chemokine C motif receptor 1 | AAGGTGCTTTCTTCG |
| | (XCR1) gene, complete cds | GGCTGTGATTATTCC |
| | | GAACTCACGTGGTAC |
| SEQ ID | GI 308210781 NG_027799 Homo sapiens | ACCTTAGCCACTCCA |
| NO: 2332 | interferon related developmental regulator 1 | CAGCCACTTTCAGGG |
| | (IFRD1), RefSeqGene on chromosome 7 | ATCAGTGTGGAGAAA |
| | | GATTCATAGTCAGGT |
| SEQ ID | GI 300116221 NG_023260 Homo sapiens | AGAAGCTCATAACAG |
| NO: 2333 | oxoglutarate dehydrogenase | TGGTGAACATTGGAG |
| | (OGDH), RefSeqGene on chromosome 7 | AGGGCAGCTGAGACC |
| | | TCAACCTTGCAAAAT |
| SEQ ID | GI 114155149 NM_020311 Homo sapiens | CATTCTCTCTTTCTC |
| NO: 2334 | atypical chemokine receptor 3 | TTGATGACGCAGCTG |
| | (ACKR3), mRNA | TCATTTGGCTGTGCG |
| | | TGCTGACAGTTTTGC |
| SEQ ID | GI 261337164 NM_206943 Homo sapiens | TCTGCGCAAATGGTG |
| NO: 2335 | latent transforming growth | ATTGTTCCAACCTTG |
| | factor beta binding protein 1 | AAGGCTCCTACATGT |
| | (LTBP1), transcript variant 1, mRN [symbol] | GTTCATGCCACAAAG |
| SEQ ID | GI 163644319 NM_032873 Homo sapiens | CCCCATACACCAGTG |
| NO: 2336 | ubiquitin associated and SH3 | TTGCTGGCTGAAGAT |
| | domain containing B | ACCCGCTTAAGGATG |
| | (UBASH3B), mRNA | CTAACATTTACCCTG |
| SEQ ID | GI 117606543 EF064746 Homo sapiens | AATCCATTCCTCCTG |
| NO: 2337 | complement component | TTAGGCCTCTGCCTA |
| | (3d/Epstein Barr virus) receptor 2 | AAATACTTGTACCCA |
| | (CR2) gene, complete cds | GCAATTTGCCTGGCT |
| SEQ ID | GI 37953284 AY438977 Homo sapiens | CCAAGAAGCTGGACA |
| NO: 2338 | phospholipase A2, group IB | GCTGTAAATTTCTGC |
| | (pancreas) | TGGACAACCCGTACA |
| | (PLA2G1B) gene, complete cds | CCCACACCTATTCAT |
| SEQ ID | GI 341865606 NG_029554 Homo sapiens | TAGACTAAACAAAAT |
| NO: 2339 | Fc fragment of IgE receptor II | CAGCAAATGGCCCCT |
| | (FCER2), RefSeqGene on chromosome 19 | GAGTGCCACCAAGTC |
| | | CCAGATGCTATCCTG |
| SEQ ID | GI 298919225 NG_023030 Homo sapiens | TTACATGCCACTGTT |
| NO: 2340 | heme oxygenase 1 | GGGGCTCAGAAAGTG |
| | (HMOX1), RefSeqGene on chromosome 22 | ATACCAGGACAACTG |
| | | GTGCTTTGACATGAT |
| SEQ ID | GI 1024336778 NG_046901 Homo sapiens | CTTAGCTCAGTCTCA |
| NO: 2341 | NLR family pyrin domain containing 9 | TTAGGCAGAACTGAG |
| | (NLRP9), RefSeqGene on chromosome 19 | CCACATCTCCCCACA |
| | | GATCCAACACAGACA |
| SEQ ID | GI 1154345445 NG_033859 Homo sapiens | GTCAGCATCCTCACC |
| NO: 2342 | sodium voltage-gated | ATCACCATGAAGGGT |
| | channel alpha subunit 11 | AAGTTCCACATCCCA |
| | (SCN11A), RefSeqGene on chromosome 3 | ATCCAAGGGAAAGTC |
| SEQ ID | GI 238776810 NG_012247 Homo sapiens | ATGGGGAAGTACTCT |
| NO: 2343 | solute carrier family 25 member 13 | CTTCCCTTGGGGTGG |
| | (SLC25A13), RefSeqGene on chromosome 7 | CTGGGACTATGTGAC |
| | | TTTTGAGTAACAGTT |
| SEQ ID | GI 1032528811 NG_050748 Homo sapiens | CCATACCCTGCTCTA |
| NO: 2344 | solute carrier family 4 member 3 | TTCCAGAAACTCTCC |
| | (SLC4A3), RefSeqGene on chromosome 2 | AGCCTGCCAAGTTAG |
| | | GTTTTCCAAGGCAAA |
| SEQ ID | GI 345842418 NG_029946 Homo sapiens | GCAAGTAAGTTTTGC |
| NO: 2345 | SMAD family member 2 | TCCACAAATCCCCCA |
| | (SMAD2), RefSeqGene on chromosome 18 | ACTCAGCAGCACATG |
| | | TCTTTTGCTCTACCC |
| SEQ ID | GI 90577165 NM_032587 Homo sapiens | CATCCCATGTGCAAG |
| NO: 2346 | caspase recruitment domain family member 6 | AGCTCTCAGTTCAAA |
| | (CARD6), mRNA | TCCGATCAGTCCAAC |
| | | CCATCCACAGTCAAA |
| SEQ ID | GI 436408720 NM_014226 Homo sapiens | TGGAGTAAATGAACT |
| NO: 2347 | MOK protein kinase | GGACCAAATCTCAAA |
| | (MOK), transcript variant 1, mRNA | AATCCACGATGTCAT |
| | | CGGCACACCCGCTCA |
| SEQ ID | GI 219802033 NM_001143835 Homo sapiens | TACGACATTGGACGA |
| NO: 2348 | nuclear factor related to | AAGCTGTGGATCTAC |
| | kappaB binding protein | CTGCATCGTGACCGG |
| | (NFRKB), transcript variant 1, mRNA | AGTGAAGAAGAGTTT |
| SEQ ID | GI 227499618 NM_003645 Homo sapiens | GCTGGTGTCGCCAGA |
| NO: 2349 | solute carrier family 27 member 2 | ACTACAAGCAGCTGT |
| | (SLC27A2), transcript variant 1, mRNA | CGAAGAGATACTGCC |
| | | AAGCCTTAAAAAAGA |
| SEQ ID | GI 166158920 NM_013996 Homo sapiens | ATGGGCAAACGGGAT |
| NO: 2350 | tachykinin precursor 1 | GCTGATTCCTCAATT |
| | (TAC1), transcript variant alpha, mRNA | GAAAAACAAGTGGCC |
| | | CTGTTAAAGGCTCTT |
| SEQ ID | GI 259013227 NG_012923 Homo sapiens | GCGTACCCTTGAGAC |
| NO: 2351 | glycogen synthase 1 | AAGGGGTCTTTGGAA |
| | (GYS1), RefSeqGene on chromosome 19 | ACTATGTCCAGGACC |
| | | TGGGAACATGAAAAC |
| SEQ ID | GI 221136810 NM_006931 Homo sapiens | TGGGTTCTGATATTG |
| NO: 2352 | solute carrier family 2 member 3 | CCGCACTAGGGGATA |
| | (SLC2A3), mRNA | TAGGAGAGGAAAAGT |
| | | AAGGTGCAGTTCCCC |
| SEQ ID | GI 39725959 NM_175852 Homo sapiens | TGATGAAGCAGCAAG |
| NO: 2353 | taxilin alpha | AGACCCACCTGAAGC |
| | (TXLNA), mRNA | AACAGCTTGCCCTAT |
| | | ACACAGAGAAGTTTG |
| SEQ ID | GI 197245401 NG_008401 Homo sapiens | GGAACTTTCTTCACC |
| NO: 2354 | glial fibrillary acidic protein | ACTTTCCCCTCCCTT |
| | (GFAP), RefSeqGene on chromosome 17 | TTCCATCTTCCTTCA |
| | | GATAGACATGCGCCC |
| SEQ ID | GI 1050193335 NG_027721 Homo sapiens | CCATAACTTGGATAG |
| NO: 2355 | transforming growth factor beta 2 | TTGCTGAGCCAGCCA |
| | (TGFB2), RefSeqGene on chromosome 1 | GATATAACAAGAGCC |
| | | ACGTGCTTTCTGGGG |
| SEQ ID | GI 518828582 NM_001278579 Homo sapiens | AAATGTTGCTCTGGG |
| NO: 2356 | activin A receptor type 2A | AGACTTACTGCATTG |
| | (ACVR2A), transcript variant 1, mRNA | CCGACAGCACAGATG |
| | | TGAAGGACATGAGAC |
| SEQ ID | GI 167614492 NM_016583 Homo sapiens | ATGAGGTTCTCAGAG |
| NO: 2357 | BPI fold containing family A member 1 | GCTTGGACATCACCC |
| | (BPIFA1), transcript variant 1, mRNA | TGGTGCATGACATTG |
| | | TTAACATGCTGATCC |
| SEQ ID | GI 345110626 NM_002001 Homo sapiens | AGTTGAAGACAGTGG |
| NO: 2358 | Fc fragment of IgE receptor la | AACCTACTACTGTAC |
| | (FCERIA), mRNA | GGGCAAAGTGTGGCA |
| | | GCTGGACTATGAGTC |
| SEQ ID | GI 83026426 DQ301957 Homo sapiens | CAAACACATGACATA |
| NO: 2359 | dual specificity phosphatase 1 | AGGCAGGTGTGTGCT |
| | (DUSP1) gene, complete cds | ACCTGCCCCTAGAGA |
| | | AACTCTTGAGCAAGG |
| SEQ ID | GI 190358485 NG_008035 Homo sapiens | CATCATACTCAGATC |
| NO: 2360 | carnitine palmitoyltransferase 2 | CAGGGTCCACCCAAA |
| | (CPT2), RefSeqGene on chromosome 1 | TTTCTCTTCTCCTCC |
| | | TCACCACTGGCTACC |
| SEQ ID | GI 190358507 NG_008042 Homo sapiens | CATCCAGGACCTGGT |
| NO: 2361 | 2,4-dienoyl-CoA reductase 1 | TTAACTAGTCCTCAT |
| | (DECR1), RefSeqGene on chromosome 8 | TCTGGTCCTGTGATG |
| | | GAACTCTGCCTCCAG |
| SEQ ID | GI 260593660 NG_013020 Homo sapiens | AAGAAGTACCTCCCA |
| NO: 2362 | eosinophil peroxidase | GCTCCAGCTGCTTCA |
| | (EPX), RefSeqGene on chromosome 17 | TGTCTCTCCAGAACT |
| | | CTGTTTCCTGACAAA |
| SEQ ID | GI 429535870 NG_033021 Homo sapiens | GTAAGCACATTCTCC |
| NO: 2363 | interleukin 17A | CCTCCTTCTTCCTTG |
| | (IL17A), RefSeqGene on chromosome 6 | CCAGAAAGCAACAAC |
| | | AAGGAATCCTCCACT |
| SEQ ID | GI 225903414 NG_011740 Homo sapiens | CCACAACATGGTGCT |
| NO: 2364 | matrix metallopeptidase 1 | TCCCTCTTCAAAGAA |
| | (MMP1), RefSeqGene on chromosome 11 | GAATCCTAGAACTCC |
| | | CTCTGCAGCATGTGG |
| SEQ ID | GI 648216382 NG_029573 Homo sapiens | AGGAAGTCCCTGAAT |
| NO: 2365 | TNF receptor superfamily member 8 | TTCCTGAACCAAGTT |
| | (TNFRSF8), RefSeqGene on chromosome 1 | GGGACCTTGTGTCTT |
| | | GCTCAGCATCACTTT |
| SEQ ID | GI 197313788 NM_001423 Homo sapiens | GTTCTGGCAAGAGCA |
| NO: 2366 | epithelial membrane protein 1 | GATACTGTCTTTGTG |
| | (EMP1), mRNA | CTGAATACGCTAAGC |
| | | CTGGAAGCCATCCTG |
| SEQ ID | GI 157909838 NM_006252 Homo sapiens | ATAGCAGACAGCCCC |
| NO: 2367 | protein kinase AMP-activated | AAAGCAAGATGTCCA |
| | catalytic subunit alpha 2 | TTGGATGCACTGAAT |
| | (PRKAA2), mRNA | ACGACTAAGCCCAAA |
| SEQ ID | GI 68160936 NM_005082 Homo sapiens | CTGGAGAAAGCATCA |
| NO: 2368 | tripartite motif containing 25 | AAACTGCGAGGAATC |
| | (TRIM25), mRNA | TCAACAAAGCCAGTC |
| | | TACATCCCCGAGGTG |
| SEQ ID | GI 31164 X51956 Human ENO2 gene | TTCTAAGTTGGACGT |
| NO: 2369 | for neuron specific | CTCCGGCGTGCATTT |
| | (gamma) enolase | TATCCTAGAGCGTCC |
| | | CTTTTGGTTTGCATT |
| SEQ ID | GI 922304389 NG_042830 Homo sapiens | TCAGGGCTTTTGTGC |
| NO: 2370 | acyl-CoA dehydrogenase family member 11 | TGGCTCTTCTGTCTG |
| | (ACAD11), RefSeqGene on chromosome 3 | CTGGAATGTGCTCTG |
| | | TCTTAGTAATTGCAA |
| SEQ ID | GI 345478654 NG_029871 Homo sapiens | GGCTTCTGGCGATGC |
| NO: 2371 | activating transcription factor 3 | CATAGTGGCTTAAGG |
| | (ATF3), RefSeqGene on chromosome 1 | ATGTCAGAGCTGAGG |
| | | TTCCACCATTTTATT |
| SEQ ID | GI 834400736 NG_042182 Homo sapiens | AGTCTAAGGAGGGTG |
| NO: 2372 | eomesodermin | CTGCCTGACATGTTC |
| | (EOMES), RefSeqGene on chromosome 3 | ACTTAAGCCACTGCT |
| | | TTGGCTTTCTTTTCT |
| SEQ ID | GI 401782607 NG_032906 Homo sapiens | GGTGCATGTTAATAT |
| NO: 2373 | coagulation factor II thrombin receptor | GGACAGTGCTGGTGT |
| | (F2R), RefSeqGene on chromosome 5 | AGACAGAAAGGCAGG |
| | | TGGATGAACTTGGCT |
| SEQ ID | GI 1024846802 NG_046996 Homo sapiens | GTGCCAACAGTGTTG |
| NO: 2374 | mediator complex subunit 1 | ATCTTCCTGCCTGTT |
| | (MED1), RefSeqGene on chromosome 17 | TCTTCTTGAAATTTC |
| | | CCCAGCCAATCCCAG |
| SEQ ID | GI 332164677 NG_028910 Homo sapiens | TCTACTTGGACACAA |
| NO: 2375 | nuclear receptor subfamily 4 group A member 3 | ACTCCTGGGGCAGTA |
| | (NR4A3), RefSeqGene on chromosome 9 | GTTCTCAATTGGGCT |
| | | ACTCCACTTTGGAGT |
| SEQ ID | GI 117606489 EF064719 Homo sapiens | CAGGTCTTTGCACCC |
| NO: 2376 | interleukin 27 receptor, alpha | TGTGTTTCCTCTTCC |
| | (IL27RA) gene, complete cds | TGGAATGCTGTTCTT |
| | | CTAGCTACTGCATGG |
| SEQ ID | GI 662033938 NG_023295 Homo sapiens | TACCTGACCCTCTCT |
| NO: 2377 | acetyl-CoA carboxylase alpha | GGGAAGGATGGAGGA |
| | (ACACA), RefSeqGene on chromosome 17 | GAGTCTAAGAAGCCT |
| | | TGTGCAGTAAGTGAT |
| SEQ ID | GI 163954923 NG_007095 Homo sapiens | GCTCAGTAGTGTACA |
| NO: 2378 | aldehyde dehydrogenase 3 family member A2 | GCAAGGAAGGCACTC |
| | (ALDH3A2), RefSeqGene on chromosome 17 | TGGCTGAAACTCACT |
| | | AAGTGGGAGAATCCT |
| SEQ ID | GI 344313160 NG_029811 Homo sapiens | CAGTATCAACTTCAT |
| NO: 2379 | carbohydrate sulfotransferase 1 | AGAGCTGGCATGAGG |
| | (CHST1), RefSeqGene on chromosome 11 | GTGCGATGAGCTGAT |
| | | GGCTGTGAAAGAAGC |
| SEQ ID | GI 219521823 NG_009222 Homo sapiens | TGAATATCAAAGCCC |
| NO: 2380 | ELK1, ETS transcription factor | ATATCCACATGTTGC |
| | (ELK1), RefSeqGene on chromosome X | TAGAGGTGAGAGCAG |
| | | CTCACCCCACTACCA |
| SEQ ID | GI 312433967 NG_027936 Homo sapiens | AATTTAAGTCTGGAC |
| NO: 2381 | pannexin 1 | TTCCCAGTCTGTCAC |
| | (PANX1), RefSeqGene on chromosome 11 | CCCAGGATGTCCTTT |
| | | ATCTGACCCCAGCTT |
| SEQ ID | GI 289577082 NG_016598 Homo sapiens | AGGCCATTATCTTGC |
| NO: 2382 | phosphorylase kinase regulatory subunit beta | TCATACAAGGTCCTT |
| | (PHKB), RefSeqGene on chromosome 16 | TCCCTTTCTGCCTGG |
| | | TCCTAAGCTGTAGGA |
| SEQ ID | GI 915408 U30245 Human | ATAGACATCTCTACC |
| NO: 2383 | myelomonocytic specific protein | AAGATCTCCTGCTCC |
| | (MNDA) gene, 5′ flanking | TGCCACTCTGTGAAC |
| | sequence and complete exon 1 | AGGAAAGGAGGGAGA |
| SEQ ID | GI 189571639 NG_007991 Homo sapiens | GCCCTCGTGGTGAAG |
| NO: 2384 | acyl-CoA dehydrogenase, | GCAAAAGACCTTAAT |
| | C-2 to C-3 short chain | CCAGAGAAGCACGTT |
| | (ACADS), RefSeqGene on chromosome 12 | CATCCCTGAGAAATT |
| SEQ ID | GI 338221701 NG_029188 Homo sapiens | CTAGAAAACCTTACC |
| NO: 2385 | caspase 9 | CCAGTGGTGCTCAGA |
| | (CASP9), RefSeqGene on chromosome 1 | CCAGAGATTCGCAAA |
| | | CCAGAGGTTCTCAGA |
| SEQ ID | GI 329665040 NG_028340 Homo sapiens | GTAAGGATTGGCCTT |
| NO: 2386 | granzyme B | GGCCACTTCGAAAAG |
| | (GZMB), RefSeqGene on chromosome 14 | GGACTAGCTGTGGTT |
| | | CAGTGAAAGGCTAAT |
| SEQ ID | GI 1016841222 NG_008723 Homo sapiens | TTTCCGCTGCATCAC |
| NO: 2387 | monoamine oxidase B | AGGCTCCTCATTCTC |
| | (MAOB), RefSeqGene on chromosome X | CACATTGCAGATAAT |
| | | CACGTTTAGGACTGC |
| SEQ ID | GI 315360651 NG_028027 Homo sapiens | AAGACACTCGATACA |
| NO: 2388 | prodynorphin | TATTTAGGCCTCAGG |
| | (PDYN), RefSeqGene on chromosome 20 | ATGGTTTGTGGCGTG |
| | | GTCAGCAGACTGACT |
| SEQ ID | GI 45643118 NM_006117 Homo sapiens | GACAGGGCAACATTT |
| NO: 2389 | enoyl-CoA delta isomerase 2 | CATACACCATTTAGT |
| | (ECI2), transcript variant 1, mRNA | CACCTAGGCCAAAGT |
| | | CCGGAAGGATGCTCC |
| SEQ ID | GI 75677324 NM_014238 Homo sapiens | TGGAGTGAAGTGCAA |
| NO: 2390 | kinase suppressor of ras 1 | GCATTGCAGGTTGAA |
| | (KSR1), mRNA | GTGTCACAACAAATG |
| | | TACCAAAGAAGCCCC |
| SEQ ID | GI 29501801 AY260471 Homo sapiens | CTAACGATGTGACCT |
| NO: 2391 | BCL2-antagonist/killer 1 | CGGGCACTCAACTCT |
| | (BAK1) gene, complete cds | TTGTGCCTCACTTTT |
| | | CCTGCCTGTAAAGTG |
| SEQ ID | GI 1174251657 KY500414 Homo sapiens | TCTTTGCTGACTCTC |
| NO: 2392 | LTB gene, complete cds | TTTCCATGTTCCTGT |
| | | GATGTTGTGCTTGTG |
| | | TCCCGACATAAGCCC |
| SEQ ID | GI 37953284 AY438977 Homo sapiens | ACCGCAACTCTCTGA |
| NO: 2393 | phospholipase A2, group IB | ACCTGTCTCTTTATC |
| | (pancreas) | TGCAAGGTGCACGAC |
| | (PLA2G1B) gene, complete cds | TGATGGGACTATTCA |
| SEQ ID | GI 1049011036 AH002959 Homo sapiens | GTTCTCAGCACAGAT |
| NO: 2394 | RAF1 | ATTCTACACCTCACG |
| | (RAF1) gene, complete cds | CCTTCACCTTGAACA |
| | | CCTCCAGTCCCTCAT |
| SEQ ID | GI 533112518 NG_007159 Homo sapiens | TCTGTCAGTTAGCAC |
| NO: 2395 | chloride voltage-gated channel 5 | CCTGGTTCCACTGGT |
| | (CLCN5), RefSeqGene on chromosome X | CATGAAAGTGAGTTG |
| | | AGTCAGGAACTTCCA |
| SEQ ID | GI 270289740 NG_015895 Homo sapiens | TGAGACCAAGTTTGC |
| NO: 2396 | four and a half LIM domains 1 | CAAGCATTGCGTGAA |
| | (FHL1), RefSeqGene on chromosome X | GTGCAACAAGGTATG |
| | | CTTTCAAGGGAGTTC |
| SEQ ID | GI 298676536 NG_021477 Homo sapiens | CAGGTCACTTCCAGA |
| NO: 2397 | glutamate decarboxylase 1 | GCACTTCTGACCATT |
| | (GAD1), RefSeqGene on chromosome 2 | TGAAAAGGCCCTGCT |
| | | TTGTGAAATTTGGCA |
| SEQ ID | GI 226493744 NG_011821 Homo sapiens | GTAGAAGCTGCTTCC |
| NO: 2398 | nuclear receptor subfamily | ACTCAGAACCCTTGT |
| | 4 group A member 2 | CCTTGGGCAAATGAA |
| | (NR4A2), RefSeqGene on chromosome 2 | GTCCAACGAGTTTCA |
| SEQ ID | GI 300796511 NG_023374 Homo sapiens | GGGCCATAAGATGAG |
| NO: 2399 | retinoid X receptor beta | GAGATTCTGAGGCAG |
| | (RXRB), RefSeqGene on chromosome 6 | GCAAGTCAGCTTTCA |
| | | GGAAAGTTGTTCCTG |
| SEQ ID | GI 241666450 NG_012339 Homo sapiens | AAAAGGCTGCATGCC |
| NO: 2400 | succinate dehydrogenase | TGTGGTTCTGTTGAT |
| | complex flavoprotein subunit A | ACGACATTCTGGAAA |
| | (SDHA), RefSeqGene on chromoso [symbol] | AGGCACATCTAGGGA |
| SEQ ID | GI 237858771 NG_012125 Homo sapiens | AGCTGTGTGGTCTCC |
| NO: 2401 | selectin P | ATGCAGATCTGTCAG |
| | (SELP), RefSeqGene on chromosome 1 | CTGAGGTTGGTGTTA |
| | | GTGAATACTACAGGG |
| SEQ ID | GI 323668281 NG_023034 Homo sapiens | GCTATTGTGTAATGG |
| NO: 2402 | vascular cell adhesion molecule 1 | AGGCCATACTTGGCG |
| | (VCAM1), RefSeqGene on chromosome 1 | GTGGAGTGCAAACAG |
| | | GACTGCAAACAAATG |
| SEQ ID | GI 31563435 NM_144673 Homo sapiens | AGCAAAGGGAAAGAA |
| NO: 2403 | CKLF like MARVEL transmembrane | ATGACTTGGAGGAGG |
| | domain containing 2 | CTCCTGGTGTCTGAA |
| | (CMTM2), transcript variant 1, mRNA [symbol] | ACGGCAGTGTATTTT |
| SEQ ID | GI 183300 M63509 Human | TTCATCGCTTATGAT |
| NO: 2404 | glutathione transferase M2 | GTCCTTGAGAGAAAC |
| | (GSTM2) mRNA, complete cds | CAAGTATTTGAGCCC |
| | | AGCTGCCTGGATGCC |
| SEQ ID | GI 937628170 KP290484 Homo sapiens | AGACTCATCTCAGTG |
| NO: 2405 | OR10J3 | ACCTACACTCATCAC |
| | (OR10J3) gene, complete cds | TCCCCTACTGAACCC |
| | | TGTTGTGTACAGCCT |
| SEQ ID | GI 1139695586 NG_052809 Homo sapiens | TATTGGAGATACGAG |
| NO: 2406 | B-cell CLL/lymphoma 3 | GACCTGGAAACTTGG |
| | (BCL3), RefSeqGene on chromosome 19 | GAGTTCAAGAACAGA |
| | | GGGCCCACAAGGATC |
| SEQ ID | GI 207438642 NG_008645 Homo sapiens | GCTGAAGCAGCCTCC |
| NO: 2407 | dopamine beta-hydroxylase | AGACCTGCTGTCATG |
| | (DBH), RefSeqGene on chromosome 9 | GGTATTTAAGGACCT |
| | | AAATGTTGCTGTCAG |
| SEQ ID | GI 354725887 NG_030444 Homo sapiens | TCAGTTTCAGGTACT |
| NO: 2408 | hepatitis A virus cellular receptor 2 | CAGCAAGATCTTTAC |
| | (HAVCR2), RefSeqGene on chromosome 5 | AAGGGGCTCCCTCAT |
| | | CCTGGAATGCTCCTT |
| SEQ ID | GI 225735562 NG_011713 Homo sapiens | GCTGACTTCATCTCT |
| NO: 2409 | insulin like growth factor 1 | TCAGCAGCCTTCCTT |
| | (IGF1), RefSeqGene on chromosome 12 | CTGGTTTTACAGCCC |
| | | ACACCACAGATTCAG |
| SEQ ID | GI 974987499 NG_023044 Homo sapiens | CGCAAGTCACACACC |
| NO: 2410 | radixin | TGGTTCCCATCCTTG |
| | (RDX), RefSeqGene on chromosome 11 | AGGAATTTGCTATCT |
| | | AAAGGCCCTTGAGTT |
| SEQ ID | GI 164692348 AK314740 Homo sapiens | CAGCCAGGTACTAAA |
| NO: 2411 | cDNA, FLI95602, Homo sapiens | GGAGCTGCTGGGACA |
| | COMM domain containing 4 | GGGGATTGATTATGA |
| | (COMMD4), mRNA | GAAGATCCTGAAGCT |
| SEQ ID | GI 83367074 NM_175617 Homo sapiens | GTGCCTGATGTGGGA |
| NO: 2412 | metallothionein IE | ACAGCTCTTCTCCCA |
| | (MT1E), mRNA | GATGTAAATAGAACA |
| | | ACCTGCACAACCTGG |
| SEQ ID | GI 116069823 EF050529 Homo sapiens | ACTGAAGCGTGGATC |
| NO: 2413 | caspase 10, apoptosis-related | CCTTTTGCTGTATTT |
| | cysteine peptidase | TCTGGTCTGGGGAAT |
| | (CASP10) gene, complete cds | ATGGACTTGCTCAGC |
| SEQ ID | GI 594591631 NG_034042 Homo sapiens | AGGGAAAATAGCTTT |
| NO: 2414 | mitogen-activated protein kinase 11 | CCTTTCCCTCCTGGT |
| | (MAPK11), RefSeqGene on chromosome 22 | GAAGGAAAAAGAGGC |
| | | CACTTTAACCCAGGG |
| SEQ ID | GI 237858721 NG_012100 Homo sapiens | GTATAGCCCCCTGCC |
| NO: 2415 | matrix metallopeptidase 3 | AAGTGGCTATCAACT |
| | (MMP3), RefSeqGene on chromosome 11 | GTGTGGGCATCTTCA |
| | | GTCATAGGGATCTTA |
| SEQ ID | GI 300116221 NG_023260 Homo sapiens | GGGCGCCTTGTTTTG |
| NO: 2416 | oxoglutarate dehydrogenase | CTGCAATATGAACTT |
| | (OGDH), RefSeqGene on chromosome 7 | AAAAGCCAAAGATCC |
| | | AGCATTGCCCCTTTG |
| SEQ ID | GI 1013071127 NG_021208 Homo sapiens | AGCCCACGTGCTAAC |
| NO: 2417 | opioid receptor mu l | AGGAATTGAAAGAAG |
| | (OPRM1), RefSeqGene on chromosome 6 | TGCCATCTTTAACTG |
| | | GGTAGGGCTGACAAG |
| SEQ ID | GI 937500889 NG_032896 Homo sapiens | TATGCCACGTATCAG |
| NO: 2418 | purinergic receptor P2Y1 | GTGACAAGAGGTCTA |
| | (P2RY1), RefSeqGene on chromosome 3 | GCAAGTCTCAACAGT |
| | | TGTGTGGACCCCATT |
| SEQ ID | GI 195927007 NM_052813 Homo sapiens | CACCCCCATGCACAC |
| NO: 2419 | caspase recruitment domain family member 9 | GCCATCTGTGTAACT |
| | (CARD9), transcript variant 1, mRNA | TCAGGATCTGTTCTG |
| | | TTTCACCATGTAACA |
| SEQ ID | GI 954169997 NM_002177 Homo sapiens | TCTTCAGAAAACCTA |
| NO: 2420 | interferon omega 1 | GGGTCCAAGGTTAAG |
| | (IFNW1), mRNA | CCATATCCCAGCTCA |
| | | GTAAAGCCAGGAGCA |
| SEQ ID | GI 193211454 NM_020651 Homo sapiens | ATCTTTCTTGGGGAG |
| NO: 2421 | pellino E3 ubiquitin protein ligase 1 | AAGGCTGCCAAATGG |
| | (PELIl), mRNA | AAGACATCAGATGGA |
| | | CAGATGGATGGCTTG |
| SEQ ID | GI 928192547 NM_006472 Homo sapiens | CTCCCTGCTATATGG |
| NO: 2422 | thioredoxin interacting protein | ATGTCATTCCTGAAG |
| | (TXNIP), transcript variant 1, mRNA | ATCACCGATTGGAGA |
| | | GCCCAACCACTCCTC |
| SEQ ID | GI 678246480 NG_034237 Homo sapiens | TTTTCGTGCCTGAAC |
| NO: 2423 | cytokine receptor like factor 2 | GTTGTCACATAAACC |
| | (CRLF2), RefSeqGene on chromosome X and 7 | CCAAGGCAGCACGTC |
| | | CAAAATGCTGTAAAA |
| SEQ ID | GI 210032106 NG_008851 Homo sapiens | ACAGCTTCACCTCTT |
| NO: 2424 | interleukin 1 beta | TCCTTTCCTTCCACA |
| | (ILIB), RefSeqGene on chromosome 2 | TTGATCAAGTTGTTC |
| | | CGCTCCTGTGGATGG |
| SEQ ID | GI 237858724 NG_012101 Homo sapiens | CCAAGCCAAGGAATT |
| NO: 2425 | matrix metallopeptidase 8 | CCAAGGACTGTAAGC |
| | (MMP8), RefSeqGene on chromosome 11 | CACCAGTAGAAGCTT |
| | | TGAAGAGGCAAGGAA |
| SEQ ID | GI 237858755 NG_012119 Homo sapiens | ACTGGCTAAGACTTC |
| NO: 2426 | protein tyrosine phosphatase, | AGACAGGCTTGTGTC |
| | non-receptor type 1 | CAGACTCTTGAGAAA |
| | (PTPN1), RefSeqGene on | GCTCTGTTGGCTTCT |
| | chromosome 2 [symbol] | |
| SEQ ID | GI 237858758 NG_012120 Homo sapiens | TGTGCTGTCAGCTGT |
| NO: 2427 | RAD51 recombinase | CATTGGCAGAGAGAA |
| | (RAD51), RefSeqGene on chromosome 15 | GGGTGTGGTGGTGTT |
| | | ATGGTATAAGAGACA |
| SEQ ID | GI 1018191682 NG_012337 Homo sapiens | TCATCCTCAGCATGT |
| NO: 2428 | succinate dehydrogenase complex subunit D | TGGCCTGTCACTTTT |
| | (SDHD), RefSeqGene on chromosome 11 | AGGCTCGTCAATTTG |
| | | TGGTCACAGTACAGC |
| SEQ ID | GI 325910867 NM_004874 Homo sapiens | GACCCAGCCTTGCAT |
| NO: 2429 | BCL2 associated athanogene 4 | TTAGCTATCATGCCT |
| | (BAG4), transcript variant 1, mRNA | ACGTCCTGTCTTCCA |
| | | GTCTGTGACAGTGTA |
| SEQ ID | GI 1047600468 NM_001718 Homo sapiens | TCTCCCCTCGTCAGC |
| NO: 2430 | bone morphogenetic protein 6 | GACACCACAAAGAGT |
| | (BMP6), mRNA | TCAAGTTCAACTTAT |
| | | CCCAGATTCCTGAGG |
| SEQ ID | GI 998614224 NM_001320340 Homo sapiens | CAAAACCACATGGGT |
| NO: 2431 | small VCP interacting protein | CGCAGGAGCTAAGGG |
| | (SVIP), transcript variant 1, mRNA | GTGGTACCCAAATGT |
| | | TAGGAACAGTGTTAG |
| SEQ ID | GI 163954920 NG_007086 Homo sapiens | CATGTGAAAACACAG |
| NO: 2432 | arginase 1 | AGACACAGGGAGCGT |
| | (ARG1), RefSeqGene on chromosome 6 | GCCAAGTGAAGATGG |
| | | AGGATTGGAGTGATG |
| SEQ ID | GI 374088072 NG_032029 Homo sapiens | TTGGAGTCTTGTCTC |
| NO: 2433 | MAP kinase interacting | CCATCCCAGCACATT |
| | serine/threonine kinase 1 | GAGGATGTAGTTGGT |
| | (MKNK1), RefSeqGene on chromosome 1 [symbol] | GAATGGACTCTTCCA |
| SEQ ID | GI 1027250665 NG_047132 Homo sapiens | TAGCTGAGATTGGAA |
| NO: 2434 | peroxisome proliferator-activated | TCCATGCCTTCTGGT |
| | receptor gamma, coactivator-related 1 | ATTCAGATCCTTCTC |
| | (PPRC1), RefSeq [symbol] | GCTGCCCCATTAGCT |
| SEQ ID | GI 340523103 NG_029468 Homo sapiens | ATGAACAAGCCTCAC |
| NO: 2435 | SWI/SNF related, matrix associated, | AAGCCCCATCTCTTT |
| | actin dependent regulator of | CTCAATCCCACTTAC |
| | chromatin, subfamily d, m [symbol] | GTTTGCCTGGTTCTC |
| SEQ ID | GI 55953136 NM_002163 Homo sapiens | TAGGGTTTGGGAGAC |
| NO: 2436 | interferon regulatory factor 8 | AGCTCATCCAATCTC |
| | (IRF8), mRNA | CCAAGTCTCATGGTG |
| | | GATTTGTGACTGTGA |
| SEQ ID | GI 26000792 AY186092 Homo sapiens | GTTCGTGTGAGATCT |
| NO: 2437 | interleukin-1 receptor-associated kinase 4 | GGTGGCTTAAAAGAG |
| | (IRAK4) gene, complete cds | TCTGCTCCCCCTTTG |
| | | CCTTCCATTGTGATT |
| SEQ ID | GI 94717611 DQ520733 Homo sapiens | AGCCAATGTCTGAAG |
| NO: 2438 | UDP glucuronosyltransferase | TAGGCAGATGAAAGA |
| | 2 family, polypeptide B4 | GAAAGGGTGGAGATG |
| | (UGT2B4) gene, complete cds | GATCCTGCTCTGAGG |
| SEQ ID | ref \| NG_021140.1\| Homo sapiens | GTGCCCAGGCACCAA |
| NO: 2439 | adiponectin, CIQ and | GCTCAGGCTCAGTTA |
| | collagen domain containing | GGAAGAAAACTTGGC |
| | | AACAGGATAATAGAG |
| SEQ ID | GI 270289769 NG_015961 Homo sapiens | GCTGAAAACGTTTCA |
| NO: 2440 | biglycan | TCACGTCCACTCCTC |
| | (BGN), RefSeqGene on chromosome X | CCAGCAACAGAGGAG |
| | | AACGGATTTCATTGT |
| SEQ ID | GI 359718928 NG_031876 Homo sapiens | GTTGCTTGGGTAGTT |
| NO: 2441 | biliverdin reductase A | CAGGACTGACAGGAC |
| | (BLVRA), RefSeqGene on chromosome 7 | TGGACACCAGGTCTC |
| | | CTGTTTATCCTTGAA |
| SEQ ID | GI 401709981 NG_032894 Homo sapiens | AATGAAATCCAGGAC |
| NO: 2442 | F2R like thrombin or trypsin receptor 3 | ACGCCACTGACTTGC |
| | (F2RL3), RefSeqGene on chromosome 19 | GCCTGTCTGCCAATT |
| | | TTTCCCCATTTAAAA |
| SEQ ID | GI 1042161084 NG_046982 Homo sapiens | GACAATGATGTGAGT |
| NO: 2443 | ficolin 1 | TCTTCGAATTGTGCT |
| | (FCN1), RefSeqGene on chromosome 9 | GAGAAGTTCCAAGGA |
| | | GCCTGGTGGTACGCC |
| SEQ ID | GI 430768625 NG_033035 Homo sapiens | GTAGACTCCAGAAGT |
| NO: 2444 | kelch like family member 40 | TCCAACACTGCTGTC |
| | (KLHL40), RefSeqGene on chromosome 3 | CATCAGCCCTTCGCC |
| | | AAAATATGGATGCTG |
| SEQ ID | GI 815930042 NM_001308319 Homo sapiens | AGATTTACCAGTCTG |
| NO: 2445 | chromodomain helicase | GTGGGAATGTGGCCC |
| | DNA binding protein 9 | TCATGATAGGGATTT |
| | (CHD9), transcript variant 1, mRNA | GCTTATTGGTGCTGC |
| SEQ ID | GI 17059625 AJ420108 Homo sapiens | AGAAGAGCGGCAGTT |
| NO: 2446 | mRNA for F-box only 32 | TCGTGAGCGACCTCA |
| | (FBXO32 gene) | GCAGTTACTGCAACA |
| | | AGGAGGTATACAATA |
| SEQ ID | GI 345441799 NG_029868 Homo sapiens | GCAACCTCTACCATG |
| NO: 2447 | advanced glycosylation | CCAGCCAACTTAGTT |
| | end-product specific receptor | AGCCTTCCTGCTTGT |
| | (AGER), RefSeqGene on chromosome | CTGATCTTCCAACAC |
| SEQ ID | GI 321267517 NG_028118 Homo sapiens | GTAGCAGACAGTGGG |
| NO: 2448 | arrestin beta 1 | GGTGGCTATGATGAC |
| | (ARRB1), RefSeqGene on chromosome 11 | TATCCCTGCTTTCCA |
| | | GACAAGGAGAATGAG |
| SEQ ID | GI 226532444 NG_011794 Homo sapiens | ATGGATGGACAAAAA |
| NO: 2449 | brain derived neurotrophic factor | GTGGCAGTTTGTGGG |
| | (BDNF), RefSeqGene on chromosome 11 | CAATTTGAGCAGTCC |
| | | TTTCATTTGGCGAGA |
| SEQ ID | GI 379642970 NG_032137 Homo sapiens | GAAGACTAATTGAAC |
| NO: 2450 | collagen type IV alpha 2 chain | CCTGCCCCTCCCCTG |
| | (COL4A2), RefSeqGene on chromosome 13 | ACCTCCACTCAGATA |
| | | GCTGATTTAAGCCTC |
| SEQ ID | GI 563317866 NG_033843 Homo sapiens | TTAGGCAGGCTCCCA |
| NO: 2451 | DnaJ heat shock protein family | GTCTGCACAACCTTT |
| | (Hsp40) member C6 | TTGCTGTGTGTCGGA |
| | (DNAJC6), RefSeqGene on chromosome | ATATGTATAACTGGC |
| SEQ ID | GI 223671920 NG_009776 Homo sapiens | GACTGTGAGAGTCCA |
| NO: 2452 | epoxide hydrolase 1 | GTGGAAGGAGGATTC |
| | (EPHX1), RefSeqGene on chromosome 1 | TTTGTCTAAGCGGCA |
| | | GAGAGGGGAAATGAC |
| SEQ ID | GI 261245113 NG_013061 Homo sapiens | ATTAGTTTCCTTTGA |
| NO: 2453 | 3-hydroxymethyl-3- | GGTTTGCTTCTCGTG |
| | methylglutaryl-CoA lyase | GGCTGGTCCAGAATG |
| | (HMGCL), RefSeqGene on chromosome 1 | GAGATCCATTGGCTG |
| SEQ ID | GI 336088631 NG_029118 Homo sapiens | CTTCAAACACCCATT |
| NO: 2454 | RAR related orphan receptor C | GCAGCCACACTTGCT |
| | (RORC), RefSeqGene on chromosome 1 | TCCTTGCTATTCCTC |
| | | GAACACATCAAACCC |
| SEQ ID | GI 226437630 NG_011780 Homo sapiens | TCTCTTGCGACATGT |
| NO: 2455 | sphingomyelin phosphodiesterase 1 | GGATTACCATACCCT |
| | (SMPD1), RefSeqGene on chromosome 11 | TCTTCTTTCCCTCCA |
| | | GCTCACTTTTCCCCT |
| SEQ ID | GI 334358873 NG_029029 Homo sapiens | AACTGGTGTAGTGGG |
| NO: 2456 | sterol regulatory element | TGGTAAACCCAATGC |
| | binding transcription factor 1 | CCACAGGGGCCTTAT |
| | (SREBF1), RefSeqGene on chromoso [symbol] | TACATTGATTTCCTC |
| SEQ ID | GI 927669118 NM_001166693 Homo sapiens | GCCTATACTTCTCTA |
| NO: 2457 | AF4/FMR2 family member 1 | CCTAAGAGGGCAGGA |
| | (AFF1), transcript variant 1, mRNA | GGGAAACCCTACAGC |
| | | TCCTTGTGAGCCTAT |
| SEQ ID | GI 189491779 NM_006507 Homo sapiens | AAGCCACCTCAAGTG |
| NO: 2458 | regenerating family member 1 beta | GACAAGGCACTTACC |
| | (REGIB), mRNA | AACAGAGATTGCTGA |
| | | TTTGCTCCTTAAGCA |
| SEQ ID | GI 146045339 EF543149 Homo sapiens | TGTGCTTATTGTGAC |
| NO: 2459 | prostaglandin E synthase | CTCAGGACCTTTGCA |
| | (PTGES) gene, complete cds | CACACTGGTCTTCCT |
| | | TCCTGGAATGCTTTT |
| SEQ ID | GI 1032528813 NG_050736 Homo sapiens | TGTCTTGTGGTATTC |
| NO: 2460 | complement C3a receptor 1 | AAGCCAATCTGGTGT |
| | (C3AR1), RefSeqGene on chromosome 12 | CAGAATCATCGCAAT |
| | | GTAGGGATGGCCTGC |
| SEQ ID | GI 307611940 NG_027759 Homo sapiens | AGAACGAGCTCATCA |
| NO: 2461 | caspase recruitment domain | GGGCGAGAGACAGGA |
| | family member 11 | TGAACATGAAGGTGA |
| | (CARD11), RefSeqGene on chromosome 7 | CAGGTATTGCATTCA |
| SEQ ID | GI 974987493 NG_012830 Homo sapiens | ACAGAAGTGGCTTTG |
| NO: 2462 | DEAD-box helicase 3, X-linked | GCAAATTTGAACGTG |
| | (DDX3X), RefSeqGene on chromosome X | GTGGAAACAGTCGCT |
| | | GGTGTGACAAATCAG |
| SEQ ID | GI 807045929 NG_029892 Homo sapiens | GTTTAGTCCTTACAT |
| NO: 2463 | potassium voltage-gated | CACACAGCTAACAAA |
| | channel subfamily J member 6 | GGTTGGCAGCCACAG |
| | (KCNJ6), RefSeqGene on chromoso [symbol] | AGCTGCTCACTGTCC |
| SEQ ID | GI 270047500 NG_016132 Homo sapiens | CTATTTAACTGGCTT |
| NO: 2464 | selectin L | CAGCCTCCCCACCTT |
| | (SELL), RefSeqGene on chromosome 1 | CTTCAGCCACCTCTC |
| | | TTTTTCAGTTGGCTG |
| SEQ ID | GI 523498696 NM_002981 Homo sapiens | CAGTTGGATGGGTTC |
| NO: 2465 | C-C motif chemokine ligand 1 | AGAGGCACAGAAAAA |
| | (CCL1), mRNA | TGCTGAGGCACTGCC |
| | | CGTCAAAAAGAAAAT |
| SEQ ID | GI 194018480 NM_001945 Homo sapiens | GGGAAAGACTTCCAT |
| NO: 2466 | heparin binding EGF like growth factor | CTAGTCACAAAGACT |
| | (HBEGF), mRNA | CCTTCGTCCCCAGTT |
| | | GCCGTCTAGGATTGG |
| SEQ ID | GI 119220595 NM_016260 Homo sapiens | ATGGAGAAGGAGAAC |
| NO: 2467 | IKAROS family zinc finger 2 | AGATTAGGGCCTTCA |
| | (IKZF2), transcript variant 1, mRNA | AGTGTGAGCACTGCC |
| | | GAGTCCTTTTCCTAG |
| SEQ ID | GI 257743477 NM_181657 Homo sapiens | AGTTCTTGCTGCGGT |
| NO: 2468 | leukotriene B4 receptor | TTCTGCCCATTTTTT |
| | (LTB4R), transcript variant 1, mRNA | TCATATCCTCTGACA |
| | | GCTGCGAGGTCATCT |
| SEQ ID | GI 257743477 NM_181657 Homo sapiens | CTTCCCTGTCCTCTG |
| NO: 2469 | leukotriene B4 receptor | CTCTGTGGTACTTCC |
| | (LTB4R), transcript variant 1, mRNA | TCTCTGGCTGATTTA |
| | | GCAAACAGCACCTAG |
| SEQ ID | GI 52854080 AY764389 Homo sapiens | AGCACTACAGGCAGA |
| NO: 2470 | baculoviral lAP repeat-containing 3 | ATTGACACTAGAGCC |
| | (BIRC3) gene, complete cds | ATCAGTTTGTGACTT |
| | | CTGGGCTAGAGTGCA |
| SEQ ID | GI 1015539575 AH009651 Homo sapiens | TTCCCTCCAGAGGGA |
| NO: 2471 | xeroderma pigmentosum group C protein | ACGGAGCCAATGAAA |
| | (XPC) gene, complete cds | CTGGCATCGTAATGC |
| | | TGGATTAAAACGTTC |
| SEQ ID | GI 357197116 NG_030591 Homo sapiens | CAGTGAACTATTTCT |
| NO: 2472 | cholecystokinin B receptor | ACACAGTGGGAACTC |
| | (CCKBR), RefSeqGene on chromosome 11 | TGACAAGGGCTGACC |
| | | TGCCTCTCACACACA |
| SEQ ID | GI 209977031 NG_008839 Homo sapiens | CCTGAACTCTGACAT |
| NO: 2473 | 24-dehydrocholesterol reductase | TTCTTCTTTCCCAGG |
| | (DHCR24), RefSeqGene on chromosome 1 | CTTTCCCTTCCCCCC |
| | | TATAGGTCTGGTTCA |
| SEQ ID | GI 268370133 NG_015859 Homo sapiens | CTTGGGAGTTGTGTT |
| NO: 2474 | GATA binding protein 3 | TGAAAACAACCTCTT |
| | (GATA3), RefSeqGene on chromosome 10 | GTGGCCCTGGACTTG |
| | | GTGAAAAAGGCAACT |
| SEQ ID | GI 345110544 NG_029851 Homo sapiens | TGGTCTTGAGCGAAG |
| NO: 2475 | phospholipase D1 | GGTAGGCGAGGGCTG |
| | (PLD1), RefSeqGene on chromosome 3 | ACTTACATTTTCCAA |
| | | GGATCATTCTTCAGA |
| SEQ ID | GI 1030311320 NG_050664 Homo sapiens | AATTTTGTTATGTTG |
| NO: 2476 | sirtuin 1 | TGCGGGCTGGAGTGT |
| | (SIRT1), RefSeqGene on chromosome 10 | AGTGCCCATCCATTG |
| | | GCACGATCATAGAGT |
| SEQ ID | GI 545746351 NM_006228 Homo sapiens | TAGGGGCAGGTGAAA |
| NO: 2477 | prepronociceptin | GGAATAGGAAATTGA |
| | (PNOC), transcript variant 1, mRNA | ACCTGGGGTTTTGAC |
| | | TTGCCACTGCCATAA |
| SEQ ID | GI 324072795 NM_005402 Homo sapiens | CACACACTGACCACA |
| NO: 2478 | RAS like proto-oncogene A | CATTAGGCTGTGTCA |
| | (RALA), mRNA | CCATTGTGTGGTGTA |
| | | CCTGCTGGAAGAATT |
| SEQ ID | GI 1143077143 NG_052860 Homo sapiens | TCAAGAGCATCTTCC |
| NO: 2479 | adiponectin receptor 1 | GCATTCATACAGAAA |
| | (ADIPOR1), RefSeqGene on chromosome 1 | CTGGCAACATCTGGA |
| | | CCCATCTGCTTGGTG |
| SEQ ID | GI 532691848 NG_009095 Homo sapiens | CTGCTCTAACGTTTT |
| NO: 2480 | calcium voltage-gated | TCCCCAACCCTGTCT |
| | channel subunit alphal F | CCGATTTCAATGCCA |
| | (CACNA1F), RefSeqGene on chromosome X | TACTATTCCCAGCCC |
| SEQ ID | GI 224809264 NG_011470 Homo sapiens | TGCCTGTTGTAGTAG |
| NO: 2481 | nitric oxide synthase 2 | CAGCTCTTCTTGCTC |
| | (NOS2), RefSeqGene on chromosome 17 | TCAAAGGTGTCTCGG |
| | | TTGGGACTGTACATT |
| SEQ ID | GI 308522736 NG_027815 Homo sapiens | GTCTCTGAGGTTGGG |
| NO: 2482 | transketolase | TGTGCCACATCCTGT |
| | (TKT), RefSeqGene on chromosome 3 | ATCCTCAGTTTATCT |
| | | GTTTCGGTCTCAGGG |
| SEQ ID | GI 1084352529 NM_000755 Homo sapiens | ATCACCGTGATGGTG |
| NO: 2483 | carnitine O-acetyltransferase | TTCCACCATTTTGGA |
| | (CRAT), transcript variant 1, mRNA | AAAGACTTCCCCAAG |
| | | TCGGAGAAGCTAAGC |
| SEQ ID | GI 91984778 NM_002214 Homo sapiens | CCATAGCCTGTATGA |
| NO: 2484 | integrin subunit beta 8 | ACAGTGTTCCCTGGA |
| | (ITGB8), mRNA | GTTCTCCAGTGCTCA |
| | | GCTTGAGACCTTGAT |
| SEQ ID | GI 1114655579 NM_002969 Homo sapiens | CGTTGACCGCACACT |
| NO: 2485 | mitogen-activated protein kinase 12 | GGATGAATGGAAGCG |
| | (MAPK12), transcript variant 1, mRNA | TGTTACTTACAAAGA |
| | | GGTGCTCAGCTTCAA |
| SEQ ID | GI 115298685 NM_006099 Homo sapiens | GGCACATTCCTTTGG |
| NO: 2486 | protein inhibitor of activated STAT 3 | GTATGTTAACCTAGG |
| | (PIAS3), mRNA | CAGTGGGAGGCAAAT |
| | | GGGATGGTATGTGAG |
| SEQ ID | GI 40807361 NM_004637 Homo sapiens | CAAACACAGTTACAC |
| NO: 2487 | RAB7A, member RAS oncogene family | CCCACATATCTCTCA |
| | (RAB7A), mRNA | CACACACACACACAC |
| | | GCACACACACACACA |
| SEQ ID | GI 319890247 NM_003256 Homo sapiens | AAGCCAACAGCCAGA |
| NO: 2488 | TIMP metallopeptidase inhibitor 4 | AGCAGTATCTCTTGA |
| | (TIMP4), mRNA | CTGGTCAGGTCCTCA |
| | | GTGATGGAAAAGTCT |
| SEQ ID | GI 1049010846 AH002874 Homo sapiens | GAAATCAGCTGCTCA |
| NO: 2489 | MC-CPA gene, complete sequence; | TCAAGATAAGGGCTG |
| | and carboxypeptidase A | AGGCATAAAACTGCC |
| | (CPA3) gene, complete cds | AGAGGGTCTCAAGGC |
| SEQ ID | GI 224809230 NG_011435 Homo sapiens | AGTCCACCTGCAGAG |
| NO: 2490 | alcohol dehydrogenase 1B | ATTCCTCATTTAATT |
| | (class 1), beta polypeptide | GACATGGGCTGTGAC |
| | (ADHIB), RefSeqGene on chromosome [symbol] | CTGGACTGTGGGATA |
| SEQ ID | GI 315506987 NG_028041 Homo sapiens | GCTGGCCCTTCATGA |
| NO: 2491 | adenylate kinase 3 | GCTGAAAAATCTCAC |
| | (AK3), RefSeqGene on chromosome 9 | CCAGTATAGCTGGCT |
| | | GTTGGATGGTAAGTG |
| SEQ ID | GI 270047497 NG_016131 Homo sapiens | CCAGACTTCCTCCTA |
| NO: 2492 | connective tissue growth factor | TAAGTCTGACAGCTC |
| | (CTGF), RefSeqGene on chromosome 6 | CGTGTATGCATTGCG |
| | | TATGGGTCTTGGCTC |
| SEQ ID | GI 1033837679 NG_008338 Homo sapiens | GTGTTCAGAGATACT |
| NO: 2493 | cytochrome P450family 7 subfamily B member 1 | GGAAAGGTGGGGAAC |
| | (CYP7B1), RefSeqGene on chromosome 8 [symbol] | ACAGACAGAGGAACT |
| | | GAGAAGCATCAGCCA |
| SEQ ID | GI 344217739 NG_029805 Homo sapiens | TGTGAGACTGGAACA |
| NO: 2494 | DnaJ heat shock protein family | AGCTTGGCAATTCAA |
| | (Hsp40) member C5 | GACCGGCATTATGGC |
| | (DNAJC5), RefSeqGene on chromosome [symbol] | AGTGAGTTATGAGGG |
| SEQ ID | GI 237874181 NG_012163 Homo sapiens | AACCTAGTCAGGAAC |
| NO: 2495 | HtrA serine peptidase 2 | CTATGGAAAACAGGC |
| | (HTRA2), RefSeqGene on chromosome 2 | TCCAGCTACCACACT |
| | | GAGCATGTGTGAACT |
| SEQ ID | GI 335892866 NG_029106 Homo sapiens | CGTCAAGACAAGCCT |
| NO: 2496 | interferon regulatory factor 7 | TGTGCAAATGGGACT |
| | (IRF7), RefSeqGene on chromosome 11 | CAGTATCTTTGGGCC |
| | | ACTGGACTCATTCAT |
| SEQ ID | GI 970414659 NG_013325 Homo sapiens | GGAGAAATGGCCTAG |
| NO: 2497 | mitogen-activated protein kinase 10 | ATTCTCCCCTCTGTC |
| | (MAPK10), RefSeqGene on chromosome 4 | TCCTGGTGATATCTC |
| | | CCTCCTATTGGCTGA |
| SEQ ID | GI 341572562 NG_029524 Homo sapiens | TACACCGACCTGTTA |
| NO: 2498 | TNF superfamily member 13b | GGCTGTGGCACAGTC |
| | (TNFSF13B), RefSeqGene on chromosome 13 | AACATGGGAGTTGTA |
| | | GACAAGCTACTTGAG |
| SEQ ID | GI 238814317 NG_012266 Homo sapiens | CCATCCCGCACCCTG |
| NO: 2499 | X-box binding protein 1 | TGCTTTCTCTACTCA |
| | (XBP1), RefSeqGene on chromosome 22 | GTTACTCACTACACC |
| | | CTGCTGGACTAAAAG |
| SEQ ID | GI 30841527 AY277591 Homo sapiens | TCACAATAACCTCCC |
| NO: 2500 | GalNAc-T5 | AACCACCAGTGTCAT |
| | (GALNT5) mRNA, complete cds | CATGTGCTTTGTGGA |
| | | TGAAGTGTGGTCCAC |
| SEQ ID | GI 325588345 HQ995528 Homo sapiens | AAGGAGTGTGAGGAA |
| NO: 2501 | testicular nuclear receptor 4 | AAATTTGACCTACAG |
| | (NR2C2) mRNA, complete cds | CTGCCGGAGCAACCA |
| | | AGACTGCATCATCAA |
| SEQ ID | GI 543173138 NM_018202 Homo sapiens | TAATGTCAGCCCTCT |
| NO: 2502 | transmembrane protein 57 | CAGCCATGCAAGACA |
| | (TMEM57), transcript variant 1, mRNA | AAACACAGCACCTGG |
| | | AGAACAGCTTAAGTG |
| SEQ ID | GI 23955951 AF549495 Homo sapiens | GAAAAATGTGGGGAA |
| NO: 2503 | cyclin G2 | TCTTCTGCCAGTAGG |
| | (CCNG2) gene, complete cds | ATTGCTGTCAGCTGC |
| | | TGGGTCAGTTCTCAG |
| SEQ ID | GI 4755084 AFO17178 Homo sapiens | GATGTCAAGGCTGTT |
| NO: 2504 | pro alpha 1(1) collagen | CCACCCCCTCCACAG |
| | (COL1A1) gene, complete cds | GTTCTTACCTTCTAC |
| | | CTCTTTCCTGCTTTA |
| SEQ ID | GI 45238842 AY563103 Homo sapiens | TCTTCTCAGCACTGT |
| NO: 2505 | interleukin 2 receptor, alpha | GCTGAGGAAGTCTTT |
| | (IL2RA) gene, complete cds | AAACCTTCCAGTAGG |
| | | ACATGGGATCCCTGC |
| SEQ ID | GI 352962130 NG_030351 Homo sapiens | GCTGTAGGAAAAATG |
| NO: 2506 | eukaryotic translation initiation | CTATCTGAGTCACTG |
| | factor 2 alpha kinase 2 | CCGTCCTCAAGGCAG |
| | (EIF2AK2), RefSeqGene on chromoso [symbol] | AAAGCAATGGCAGAA |
| SEQ ID | GI 323510659 NG_028143 Homo sapiens | TTCACATTGCTTCTT |
| NO: 2507 | interleukin 18 | CTTGGTGGAAGCTCT |
| | (IL18), RefSeqGene on chromosome 11 | TCCTCTGGCCTTGAA |
| | | AATGCCTGCTTCTCT |
| SEQ ID | GI 1152003149 NG_016599 Homo sapiens | CAATTTGTAAAGCAG |
| NO: 2508 | phosphorylase kinase | GGGTGTCACTCCAAA |
| | regulatory subunit alpha 1 | AGAAGTGGGCCACTG |
| | (PHKA1), RefSeqGene on chromosome X | TTCTCACCACTAGCT |
| SEQ ID | GI 291045304 NG_016710 Homo sapiens | GTTGACCATTTTAAC |
| NO: 2509 | ribose 5-phosphate isomerase A | AAACCGGCAGAATTC |
| | (RPIA), RefSeqGene on chromosome 2 | CTGAGCCTCGTGTTG |
| | | TGAATTGGGGCACAG |
| SEQ ID | GI 402232739 NM_024295 Homo sapiens | TAAACCATGGGGGAT |
| NO: 2510 | derlin 1 | GCAACCCCTTTGCGT |
| | (DERL1), transcript variant 1, mRNA | TTCATATGTAGGCCT |
| | | ACTGGCTTTGTGTAG |
| SEQ ID | GI 514052655 NM_001278549 Homo sapiens | AACACCATGCCAACA |
| NO: 2511 | pyruvate dehydrogenase kinase 1 | GAGGTGTTTACCCCC |
| | (PDK1), transcript variant 1, mRNA | CTATTCAAGTTCATG |
| | | TCACGCTGGGTAATG |
| SEQ ID | GI 1168024004 KY497357 Homo sapiens | CACTCATCCCTACTC |
| NO: 2512 | HLA-DRA | GCCATCATTCTTTCA |
| | (HLA-DRA) gene, complete cds | TTCCTCTTGGCCCAA |
| | | TCTCTCTCCACCACT |
| SEQ ID | GI 1036030212 AH006319 Homo sapiens | TCCGCCGTTTGCTGT |
| NO: 2513 | succinate dehydrogenase subunit C | TTGGGAGTGCGATAC |
| | (SDHC) gene, complete cds | AAAATGAATATGTAC |
| | | GCAGGTGGAACGTAG |
| SEQ ID | GI 1036032775 AH003000 Homo sapiens | ATACGTAACTGCAGC |
| NO: 2514 | steroid 5-alpha-reductase | CAACTATTTTGGAGA |
| | (SRD5A1) gene, complete cds | AATCATGGAGTGGTG |
| | | TGGCTATGCCCTGGC |
| SEQ ID | GI 994323650 NG_011530 Homo sapiens | GTCCAAGCTCAGTCA |
| NO: 2515 | 2′-5′-oligoadenylate synthetase 1 | GCAGAAGAGATAAAA |
| | (OAS1), RefSeqGene on chromosome 12 | GCAAACAGGTCTGGG |
| | | AGGCAGTTCTGTTGC |
| SEQ ID | GI 194018405 NG_008205 Homo sapiens | TTTGCCCAAGTTAGG |
| NO: 2516 | phosphoenolpyruvate carboxykinase 1 | GTGCATCCTTCCCAT |
| | (PCK1), RefSeqGene on chromosome 20 | GAACTTTGACTGTGA |
| | | CCTTTGACTATGGGG |
| SEQ ID | GI 574997036 NG_033896 Homo sapiens | CCAACTTGCAAATGT |
| NO: 2517 | tubulin beta 4A class IVa | GAAATTCAGACACAG |
| | (TUBB4A), RefSeqGene on chromosome 19 | AGAGGAGCACCAGGG |
| | | CAAGGTCATACACCT |
| SEQ ID | GI 38708325 NM_001896 Homo sapiens | CTACGAGGCTCACGT |
| NO: 2518 | casein kinase 2 alpha 2 | CCCGAGCTGGGGTAA |
| | (CSNK2A2), mRNA | TCAAGATGATTACCA |
| | | ACTGGTTCGAAAACT |
| SEQ ID | GI 665505917 NM_006145 Homo sapiens | TTGTGGGTCACACTC |
| NO: 2519 | DnaJ heat shock protein family | TTTACATTTCTGTAA |
| | (Hsp40) member B1 | GGCAATCTTGGCACA |
| | (DNAJB1), transcript variant 1, mRNA | CGTGGGGCTTACCAG |
| SEQ ID | GI 588282795 NM_002168 Homo sapiens | TATTATCTGGCAGTT |
| NO: 2520 | isocitrate dehydrogenase | CATCAAGGAGAAGCT |
| | (NADP(+)) 2, mitochondrial | CATCCTGCCCCACGT |
| | (IDH2), transcript variant 1, mRNA | GGACATCCAGCTAAA |
| SEQ ID | GI 225579058 NM_197965 Homo sapiens | CTTTCCTTTGATTGG |
| NO: 2521 | solute carrier family 10 member 6 | CCATGTCACGGGTTT |
| | (SLC10A6), mRNA | TCTGCTGGCACTTTT |
| | | TACCCACCAGTCTTG |
| SEQ ID | GI 10280531 AB038161 Homo sapiens | TGGCCGGGTGACCAT |
| NO: 2522 | ABCG1 gene for ABC transporter | GTTAAGGCCATTAAA |
| | (ATP-binding cassette, sub-family G | GTCATGCTTGGAGAA |
| | (WHITE), member 1), com [symbol] | ACCACATAGATGCTT |
| SEQ ID | GI 22788696 AF511593 Homo sapiens | CTGCTTAAAGTCAGA |
| NO: 2523 | cyclin D1 | AACAGGTTGAAGGAG |
| | (PRAD1: parathyroid adenomatosis 1) | GTGGAGGCGTGGGAA |
| | (CCND1) gene, complete cds | AGAGTCTAGGAAGGT |
| SEQ ID | GI 42416963 AY534685 Homo sapiens | CTTCCCTCTAAGAAC |
| NO: 2524 | insulin-like growth factor binding protein 5 | CCCACACACAAGGAC |
| | (IGFBP5) gene, complete cds | ACTTTAGGCCACCAT |
| | | GCAAGGTTTTCAGAC |
| SEQ ID | GI 1036032766 AH002941 Homo sapiens | GAAGTGTCAGTCTTG |
| NO: 2525 | plasminogen | GTCATCTATGACACC |
| | (PLG) gene, complete cds | ACACCGGCACCAGAA |
| | | GACCCCAGAAAACTA |
| SEQ ID | GI 238018096 NG_012212 Homo sapiens | TGCCACATGTGCAAC |
| NO: 2526 | C-C motif chemokine ligand 11 | TCAGGCATTCTTGGA |
| | (CCL11), RefSeqGene on chromosome 17 | AACCATCCCTGCGGT |
| | | AGCATATCTTTACTT |
| SEQ ID | GI 284813593 NG_016440 Homo sapiens | CATGGCACACAGCTG |
| NO: 2527 | MAF bZIP transcription factor | TCTCGGTAGCTGCAT |
| | (MAF), RefSeqGene on chromosome 16 | TCCCAGCATGTCCTG |
| | | GTCTAAATATCTAGA |
| SEQ ID | GI 340805863 NG_029510 Homo sapiens | GCTCCTTTGGTGCTG |
| NO: 2528 | protein tyrosine kinase 2 beta | AACTGCACTTGGCAA |
| | (PTK2B), RefSeqGene on chromosome 8 | TAGCCCAGAATGATG |
| | | ACTGTTTTGCAGTTT |
| SEQ ID | GI 22788696 AF511593 Homo sapiens | TCTAAAGGTGAAGGG |
| NO: 2529 | cyclin D1 | ACGTCTACACCCCCA |
| | (PRAD1: parathyroid adenomatosis 1) | ACAAAACCAATTAGG |
| | (CCND1) gene, complete cds | AACCTTCGGTGGTCT |
| SEQ ID | GI 21914605 AY130302 Homo sapiens | ATGGGCTGTCTGGAC |
| NO: 2530 | truncated recombination activating gene 1 | TATCATCCTCTGTGG |
| | (RAG1) gene, complete cds | ATGATTACCCAGTGG |
| | | ACACCATTGCAAAGA |
| SEQ ID | GI 1143077143 NG_052860 Homo sapiens | CTTCCCATTTCTGGA |
| NO: 2531 | adiponectin receptor 1 | CATCCCACTATCCCC |
| | (ADIPOR1), RefSeqGene on chromosome 1 | TCATCGTCAAAATCA |
| | | AAGCCTTGCCCCTTT |
| SEQ ID | GI 1025812271 NG_047045 Homo sapiens | TATTCAGCAGGCAGT |
| NO: 2532 | activating transcription factor 2 | ACCTTCACCAACCTC |
| | (ATF2), RefSeqGene on chromosome 2 | AAGTACTGTAATCAC |
| | | CCAGGCACCATCCTC |
| SEQ ID | GI 601984942 NG_034078 Homo sapiens | TCTGGAGATCTCCAG |
| NO: 2533 | myelin associated glycoprotein | GGGCATCACGGTGAC |
| | (MAG), RefSeqGene on chromosome 19 | AAAGATAATGATCAG |
| | | CACATCATCACTGCA |
| SEQ ID | GI 237858721 NG_012100 Homo sapiens | CATGCAGCTTTCCCT |
| NO: 2534 | matrix metallopeptidase 3 | CGTGGACTTTAGACC |
| | (MMP3), RefSeqGene on chromosome 11 | CATTGGGAAAATGTT |
| | | CTGAGAACCCAGACC |
| SEQ ID | GI 237858735 NG_012106 Homo sapiens | GTAGAACCTTCTGCC |
| NO: 2535 | 8-oxoguanine DNA glycosylase | GCTCCAAGCACCTTC |
| | (OGG1), RefSeqGene on chromosome 3 | TGAGAAATAATGGAT |
| | | CCCCAAAAGCCACTG |
| SEQ ID | GI 238018085 NG_012206 Homo sapiens | ATGACCCACGTGTTT |
| NO: 2536 | protein kinase C alpha | CCCATTTGTGCTTGT |
| | (PRKCA), RefSeqGene on chromosome 17 | TATTTAGAAAGCCAA |
| | | ACTTGGCCCTGCTGG |
| SEQ ID | GI 325910849 NG_028199 Homo sapiens | CACACCTGCCTTGAT |
| NO: 2537 | scavenger receptor class B member 1 | GTGTGACGACGTGAT |
| | (SCARB1), RefSeqGene on chromosome 12 | GATGTGTATGCAGAC |
| | | ACCTGTGTTGATGTG |
| SEQ ID | GI 61676924 NM_012242 Homo sapiens | CGGTCATCAGACTGT |
| NO: 2538 | dickkopf WNT signaling pathway inhibitor 1 | GCCTCAGGATTGTGT |
| | (DKK1), mRNA | TGTGCTAGACACTTC |
| | | TGGTCCAAGATCTGT |
| SEQ ID | GI 359279909 NM_020663 Homo sapiens | TCACCACTTCCCTAA |
| NO: 2539 | ras homolog family member J | TGGGCCACAGGAAGT |
| | (RHOJ), mRNA | AAGTTGATCTTGATG |
| | | GGGAGATCACGTCAC |
| SEQ ID | GI 218083972 NM_134431 Homo sapiens | GGGAGATGCTCGAAG |
| NO: 2540 | solute carrier organic anion | ACCTCAGAATATACA |
| | transporter family member 1A2 | CACCCTTAAGGTTCA |
| | (SLCO1A2), transcript variant 1, [symbol] | AGGGCTGGGGTTGAG |
| SEQ ID | GI 117606489 EF064719 Homo sapiens | ATTTTTTTGACAGGC |
| NO: 2541 | interleukin 27 receptor, alpha | CCATGTTCTGGAAAC |
| | (IL27RA) gene, complete cds | CCCCTTGGTCCTGGG |
| | | CAAATACAAATGGTT |
| SEQ ID | GI 1800224 U70065 Human | TTTTTTTCAAAGGCA |
| NO: 2542 | JAK3 gene, complete cds | TTGGGGTTAACAGGA |
| | | TGGGGTGGTGTTTGA |
| | | GAGGGGGAGGACATA |
| SEQ ID | GI 42716353 AY549314 Homo sapiens | CAAAGTCGAGGAGTG |
| NO: 2543 | tyrosine kinase 2 | GTTATGGTGTGGCAC |
| | (TYK2) gene, complete cds | AGAAGAAGGGTCAGA |
| | | GGACGGTCTTTGTTA |
| SEQ ID | GI 189217843 NG_007930 Homo sapiens | GGGACTTACAGGGAG |
| NO: 2544 | cytochrome P450 family 2 | ATCAGGGAGAGAAGC |
| | subfamily F member 1 | AGAGAGAAGGAGAAA |
| | (CYP2F1), RefSeqGene on chromosome 19 | GCCCAGAGAGCATAA |
| SEQ ID | GI 1025812269 NG_047054 Homo sapiens | CATTCCTCTTCAGGA |
| NO: 2545 | glutathione peroxidase 5 | ATTGCCTCAGCCTAA |
| | (GPX5), RefSeqGene on chromosome 6 | GTTTTTCCCCTTTCC |
| | | AGGGCAGCCTGTATC |
| SEQ ID | GI 293597500 NG_016966 Homo sapiens | GAAAGAGCCTGATCT |
| NO: 2546 | phosphoglucomutase 1 | GGAAGGGGAGTTCTT |
| | (PGM1), RefSeqGene on chromosome 1 | CCACCAGATGTACCT |
| | | CTTCTTGGGCTCAAG |
| SEQ ID | GI 746817461 NG_008863 Homo sapiens | GCACAATGCTTAAGG |
| NO: 2547 | proteolipid protein 1 | CAGTGTTCCAGTGCT |
| | (PLP1), RefSeqGene on chromosome X | GGTGTAGGCTGACCA |
| | | ACTTGTTTTGGTTTT |
| SEQ ID | GI 209977032 NG_008779 Homo sapiens | CTCTTGGTCCAGTCC |
| NO: 2548 | paraoxonase 1 | TTTCTCTCCACATTG |
| | (PON1), RefSeqGene on chromosome 7 | CTGGATTACTGCTGT |
| | | AGCCTCAGCTCATGT |
| SEQ ID | GI 215422398 NG_009053 Homo sapiens | CCAGAAGATACCCTG |
| NO: 2549 | protein activator of interferon | GCTGTTAAACTAGCC |
| | induced protein kinase EIF2AK2 | CTGCTGGGCAAATGA |
| | (PRKRA), RefSeqGene on chro [symbol] | AAAGGCACCTACATT |
| SEQ ID | GI 323422857 NM_001565 Homo sapiens | CTGCCATCCTCCCAA |
| NO: 2550 | C-X-C motif chemokine ligand 10 | GGGGCCCAAATTCTT |
| | (CXCL10), mRNA | TCAGTGGCTACCTAC |
| | | ATACAATTCCAAACA |
| SEQ ID | GI 28178835 NM_005530 Homo sapiens | TAGCAGCCGGTCACC |
| NO: 2551 | isocitrate dehydrogenase 3 | CATCTATGAATTTAC |
| | (NAD(+)) alpha | TGCTGCGCAAAACAT |
| | (IDH3A), mRNA | TTGACCTTTACGCGA |
| SEQ ID | GI 594140701 NM_002661 Homo sapiens | TTGATGACACCATGC |
| NO: 2552 | phospholipase C gamma 2 | GTGAAACTGCTGAGC |
| | (PLCG2), mRNA | CTTTCTTGTTTGTGG |
| | | ATGAGTTCCTCACGT |
| SEQ ID | GI 187473261 EU595874 Homo sapiens | CTCTGAGTCATATCA |
| NO: 2553 | carboxylesterase 3 | TTCTCCTGCTCGAAA |
| | (brain) | GCTCCTGGTGACTCC |
| | (CES3) gene, complete cds | CTGTCTCCCTGAAGA |
| SEQ ID | GI 14488407 AF386492 Homo sapiens | CTCTCTCAGTCCCTC |
| NO: 2554 | serine-cysteine proteinase | TGCATCAAGACACTT |
| | inhibitor clade E member 1 | ACCCATTTCCCACCT |
| | (SERPINEl) gene, complete cds | CATGCCTGCTAACTT |
| SEQ ID | GI 237858689 NG_012090 Homo sapiens | CCAAAGCCCACACAC |
| NO: 2555 | interleukin 13 | TTAACCACCCTGCCT |
| | (IL13), RefSeqGene on chromosome 5 | ATTTCTGTCCAAATG |
| | | TCACCTGTCCTGAAT |
| SEQ ID | GI 296010831 NG_021194 Homo sapiens | TCACTGTTGGTTCGT |
| NO: 2556 | interleukin 1 receptor associated kinase 3 | GTCTTCAGGTAGGTC |
| | (IRAK3), RefSeqGene on chromosome 12 | CAGGAAGTGGGCTCA |
| | | GAAGCACAGATTTAC |
| SEQ ID | GI 296010831 NG_021194 Homo sapiens | GCACTGTGATCTTGG |
| NO: 2557 | interleukin 1 receptor associated kinase 3 | GCAAGTCACTTAATC |
| | (IRAK3), RefSeqGene on chromosome 12 | CCCTGAGGCTCAGTC |
| | | TCTTACCTCATCTGG |
| SEQ ID | GI 210032137 NG_008855 Homo sapiens | GTAGACGTCTTACAC |
| NO: 2558 | lipoprotein lipase | ACATTCACCAGAGGG |
| | (LPL), RefSeqGene on chromosome 8 | TCCCCTGGTCGAAGC |
| | | ATTGGAATCCAGAAA |
| SEQ ID | GI 218749810 NG_009188 Homo sapiens | TAACTTGACTGCTGG |
| NO: 2559 | phosphoglycerate dehydrogenase | GCATTTTTGGACTGT |
| | (PHGDH), RefSeqGene on chromosome 1 | TTGGGAGGGCTCAGC |
| | | TTTCTTGTCTGTCTT |
| SEQ ID | GI 294489300 NG_017057 Homo sapiens | ACAGTAAACCTGGGC |
| NO: 2560 | solute carrier family 27 member 4 | CACTCCAGGGTTACT |
| | (SLC27A4), RefSeqGene on chromosome 9 | GTGAAGGTGACATGA |
| | | GCTGATGTGTATCAG |
| SEQ ID | GI 323276689 NG_028137 Homo sapiens | TCAGATGGACACTGA |
| NO: 2561 | secretory leukocyte peptidase inhibitor | GATGGCCTCTTTAAT |
| | (SLPI), RefSeqGene on chromosome 20 | CAACCAACTTCCCAG |
| | | GCCAATCTCTTCCCT |
| SEQ ID | GI 68215223 NM_003965 Homo sapiens | GCTGGCACCAGAGGA |
| NO: 2562 | C-C motif chemokine receptor like 2 | TGAATATGATGTCCT |
| | (CCRL2), transcript variant 1, mRNA | CATAGAAGGTGAACT |
| | | GGAGAGCGATGAGGC |
| SEQ ID | GI 1128611453 NM_019058 Homo sapiens | CGCATGAATGTAAGA |
| NO: 2563 | DNA damage inducible transcript 4 | GTAGGAAGGGGTGGG |
| | (DDIT4), mRNA | TGTCAGGGATCACTT |
| | | GGGATCTTTGACACT |
| SEQ ID | GI 105990527 NM_012258 Homo sapiens | TTTGAGAAGCAGGGA |
| NO: 2564 | hes related family Bhlh | TCTGCTAAGCTAGAA |
| | transcription factor with YRPW motif 1 | AAAGCCGAGATCCTG |
| | (HEY1), transcript variant 1, | CAGATGACCGTGGAT |
| SEQ ID | GI 392996937 NM_001008387 Homo sapiens | GCTATGCCTTGTTTT |
| NO: 2565 | regenerating family member 3 gamma | TGTCACCAAAATCCT |
| | (REG3G), transcript variant 1, mRNA | GGATGGATGCAGATC |
| | | TGGCTTGCCAGAAGC |
| SEQ ID | GI 2735714 AF004877 Homo sapiens | GCCATCCTTAAGAGG |
| NO: 2566 | pro-alpha 2(1) collagen | ACTGAAGCAGGTTAT |
| | (COL1A2) gene, complete cds | AGAGGAATCGCAGCT |
| | | GTGCACTCCCACTAC |
| SEQ ID | GI 50234988 AY675320 Homo sapiens | CTGCATGAAGCACCA |
| NO: 2567 | RAD21 homolog | AGCAAGCCCTACCTA |
| | (S. pombe) | TTCACAAAGCAGGAA |
| | (RAD21) gene, complete cds | AGAGGGAAGGAAGAG |
| SEQ ID | GI 166235119 NG_007149 Homo sapiens | CCAATACTGTCGAGT |
| NO: 2568 | B-cell CLL/lymphoma 6 | GCCATTCTGCCTTTT |
| | (BCL6), RefSeqGene on chromosome 3 | GAAGACAAGTGCACC |
| | | TTCTTTCCCTTCCCA |
| SEQ ID | GI 953768433 NG_016863 Homo sapiens | CAGATGCGATCGTGT |
| NO: 2569 | delta like non-canonical Notch ligand 1 | GTGTGTGGGGACTGC |
| | (DLK1), RefSeqGene on chromosome 14 | GTATATGTTTTTGTC |
| | | AGTGTGTGTGACCAC |
| SEQ ID | GI 214010161 NG_009030 Homo sapiens | GACAACTAAGGCCCT |
| NO: 2570 | GLI family zinc finger 2 | GTAGGTTGATTCCAG |
| | (GLI2), RefSeqGene on chromosome 2 | AATACACTATCCAGG |
| | | TCCAGGGTGCAGTGG |
| SEQ ID | GI 237874160 NG_012149 Homo sapiens | TTGGACATTTGTTCC |
| NO: 2571 | isocitrate dehydrogenase 3 | TTCCCATTGGGAGCC |
| | (NAD(+)) beta | CAGGAGTTGAGAGAT |
| | (IDH3B), RefSeqGene on chromosome 20 | CATGTCAGCTGTCTA |
| SEQ ID | GI 21435968 AF518727 Homo sapiens | AAGTGGATGGTTCCA |
| NO: 2572 | cyclin El | TTTGCCATGGTTATA |
| | (CCNEl) gene, complete cds | AGGGAGACGGGGAGC |
| | | TCAAAACTGAAGCAC |
| SEQ ID | GI 67515430 DQ088982 Homo sapiens | TACCCTGTCTCCCAC |
| NO: 2573 | glutathione peroxidase 6 | CTGCAGGAATGTCTA |
| | (olfactory) | ACAAAGCATCCATCT |
| | (GPX6) gene, complete cds | TCCTCCTTCTTTGCT |
| SEQ ID | GI 225735572 NG_011717 Homo sapiens | CTCCTGATTGCAGAG |
| NO: 2574 | alpha-2-macroglobulin | CTCCATCTACTCCTG |
| | (A2M), RefSeqGene on chromosome 12 | GAGCTAGGTGATTTA |
| | | GGAGCCAGACCCTTA |
| SEQ ID | GI 339515631 NG_029366 Homo sapiens | TTAGGCCCAGCGAGA |
| NO: 2575 | coagulation factor III, tissue factor | GAGAACAGTTTCTCA |
| | (F3), RefSeqGene on chromosome 1 | AGGTAGGAATGGTGA |
| | | AAAGAAGGGGTGGAC |
| SEQ ID | GI 1135567463 NG_052785 Homo sapiens | ACAGGTAAGATGACA |
| NO: 2576 | galanin and GMAP prepropeptide | CTGTCATGATCATCA |
| | (GAL), RefSeqGene on chromosome 11 | TTCGTCCTGTGCCCT |
| | | GTGGACATCAACCCA |
| SEQ ID | GI 1216866539 NG_012872 Homo sapiens | GTCCTCATCTAGAAC |
| NO: 2577 | gelsolin | CAAAGGAGAGGGGCT |
| | (GSN), RefSeqGene on chromosome 9 | CCAAAAGAATCACCA |
| | | GTGAGGATTGGGTGT |
| SEQ ID | GI 818213488 NG_042142 Homo sapiens | CAAAAGCCCTTCCTC |
| NO: 2578 | inositol 1,4,5-trisphosphate receptor type 2 | ATCTGAGCTGGTCTA |
| | (ITPR2), RefSeqGene on chromosome 12 | GTCTGACCTTTTCCC |
| | | ATCACCCTCCATCTC |
| SEQ ID | GI 226061859 NG_011749 Homo sapiens | GACGTTCACAGCTGA |
| NO: 2579 | peroxisome proliferator | ATCTCAGTGAACAGA |
| | activated receptor gamma | ATAGCAGTCCCTGGA |
| | (PPARG), RefSeqGene on chromosome 3 [symbol] | TGGCAGTCTGCCTAA |
| SEQ ID | GI 294489300 NG_017057 Homo sapiens | GAGATTGAAAAAGTC |
| NO: 2580 | solute carrier family 27 member 4 | GAGCTCTTGCCAGGA |
| | (SLC27A4), RefSeqGene on chromosome 9 | GAAGTGCCAAAGCCA |
| | | CATAGCAGAGGTGTG |
| SEQ ID | GI 674651085 NG_012882 Homo sapiens | CCAAGCCATTCTTTG |
| NO: 2581 | toll like receptor 8 | ACTGCTGACAGGCCT |
| | (TLR8), RefSeqGene on chromosome X | TCCTTCATAACAAGG |
| | | TGTTCCACAGTCCAT |
| SEQ ID | GI 323362950 NM_138621 Homo sapiens | ATGAGTTGTGACAAA |
| NO: 2582 | BCL2 like 11 | TCAACACAAACCCCA |
| | (BCL2L11), transcript variant 1, mRNA | AGTCCTCCTTGCCAG |
| | | GCCTTCAACCACTAT |
| SEQ ID | GI 1040975354 NM_001737 Homo sapiens | AAGCTTCTCCGAGGA |
| NO: 2583 | complement C9 | ACCGTGATTGATGTG |
| | (C9), mRNA | ACTGACTTTGTCAAC |
| | | TGGGCCTCTTCCATA |
| SEQ ID | GI 40807361 NM_004637 Homo sapiens | ATCCAGGCCAGTCCC |
| NO: 2584 | RAB7A, member RAS oncogene family | CGAGATCCTGAAAAC |
| | (RAB7A), mRNA | TTCCCATTTGTTGTG |
| | | TTGGGAAACAAGATT |
| SEQ ID | GI 632794850 NM_002957 Homo sapiens | TACGATTGTCTCTGA |
| NO: 2585 | retinoid X receptor alpha | AACATCGTGGCCTCA |
| | (RXRA), transcript variant 1, mRNA | GGTGCCAGGGTTTGA |
| | | TGGACAGTAGCATTA |
| SEQ ID | GI 1024846747 NG_047015 Homo sapiens | TGGGGCAAACACTCA |
| NO: 2586 | bone gamma-carboxyglutamate protein | TATTCCTGAGGGATA |
| | (BGLAP), RefSeqGene on chromosome 1 | CAGGTCCCCAAATTT |
| | | ATGGCTCCTGGATCT |
| SEQ ID | GI 299782537 NG_023178 Homo sapiens | TACATGGTCTAACCC |
| NO: 2587 | CD14 molecule | CTGCATACCTCTCCA |
| | (CD14), RefSeqGene on chromosome 5 | ATCTGGACTCACCTC |
| | | TTCTGGGACCTTCTC |
| SEQ ID | GI 807045929 NG_029892 Homo sapiens | TCCATCATCACATCA |
| NO: 2588 | potassium voltage-gated | CCCTTTTTCTTTTCT |
| | channel subfamily J member 6 | GTCCCTCCCGCCCTA |
| | (KCNJ6), RefSeqGene on chromosor [symbol] | CCCTTATAAGGACCC |
| SEQ ID | GI 162329557 NG_007161 Homo sapiens | CCTGAGGGAGAGTGA |
| NO: 2589 | MYC proto-oncogene, bHLH transcription factor | GGTTAAGTTCTGAAC |
| | (MYC), RefSeqGene on chromosome 8 | CCACAGGCATTATAT |
| | | CTGCCTGGGGTTCAC |
| SEQ ID | GI 353249887 NG_030361 Homo sapiens | ATCTTGTTACCCGGG |
| NO: 2590 | Spl transcription factor | TCCGGTCATGAGCAT |
| | (SP1), RefSeqGene on chromosome 12 | AGCAACCCAACAGTT |
| | | TTTCCAACATTACCC |
| SEQ ID | GI 338797748 NM_006743 Homo sapiens | CATCCAACTGATGCA |
| NO: 2591 | RNA binding motif | CATTTGTCAAGAACC |
| | (RNP1, RRM) protein 3 | CACTGAGGCTAGAAA |
| | (RBM3), mRNA | CCCCCCAAACACTCA |
| SEQ ID | GI 4455117 AF125158 Homo sapiens | GAAGCCAAGTGCATC |
| NO: 2592 | zinc finger DNA binding protein 99 | TTCCAAACCTTCTTT |
| | (ZNF281) mRNA, complete cds | GGTTGGAGATGGAGA |
| | | AGGTGCCATCCTCTC |
| SEQ ID | GI 189217843 NG_007930 Homo sapiens | ATGGCAAATTCTCTC |
| NO: 2593 | cytochrome P450 family 2 subfamily F member 1 | CCACCTTCCTCACCT |
| | (CYP2F1), RefSeqGene on chromosome 19 [symbol] | CCAGATCCCACCACT |
| | | TTCCTGAATTGTACT |
| SEQ ID | GI 342837712 NG_029640 Homo sapiens | CTTGTGTCTGGGCCA |
| NO: 2594 | mitogen-activated protein | TTTCTCTCACTGTGT |
| | kinase-activated protein kinase 2 | AGGAAAGAGTGGTTC |
| | (MAPKAPK2), RefSeqGene on c [symbol] | CTGTCTTCTCCCTGG |
| SEQ ID | GI 342837712 NG_029640 Homo sapiens | GTGCAAGTCTGGAAG |
| NO: 2595 | mitogen-activated protein | TGTGATGTGAACCTA |
| | kinase-activated protein kinase 2 | GCCCTTCTTGCACAA |
| | (MAPKAPK2), RefSeqGene on c [symbol] | ACTCTCCTTTGCCTT |
| SEQ ID | GI 341572552 NG_029517 Homo sapiens | GCCTAATGCTGTGAT |
| NO: 2596 | retinoid X receptor gamma | ATATGCTGACTTGCA |
| | (RXRG), RefSeqGene on chromosome 1 [symbol] | CAGTGACCCCAAGAT |
| | | CTTCCTGTTGGCAGC |
| SEQ ID | GI 193290120 NG_008160 Homo sapiens | GCTGTGATGTATGCT |
| NO: 2597 | transaldolase 1 | GCTGCTCTGAACACA |
| | (TALD01), RefSeqGene on chromosome 11 [symbol] | TCTGCTGCTATGAAC |
| | | ACATCTGCTGCTCTG |
| SEQ ID | GI 19718735 NM_003265 Homo sapiens | ACGCAAACCCTGGTG |
| NO: 2598 | toll like receptor 3 | GTCCCATTTATTTCC |
| | (TLR3), mRNA [symbol] | TAAAGGGTCTGTCTC |
| | | ACCTCCACATCCTTA |
| SEQ ID | GI 117606575 EF064762 Homo sapiens | TCAGTTAAAATCAAG |
| NO: 2599 | chemokine | TGGGGCATGGGGGAT |
| | (C-C motif) ligand 27 | GGAGGTATGGGGAGG |
| | (CCL27) gene, complete cds [symbol] | TAGTTCCAGAATCAG |
| SEQ ID | GI 39654020 AY496422 Homo sapiens | GGGCCACTGAGCACT |
| NO: 2600 | nuclear factor of kappa light polypeptide | ATTCACCCTCACCTT |
| | gene enhancer in B-cells inhibitor, alpha | TTACTTCACATCAGC |
| | (NFKBIA) gen [symbol] | CCACATCCTAGAGAG |
| SEQ ID | ref\|NG_052974.1\| Homo sapiens | CTTCCATGTCAGTGC |
| NO: 2601 | cyclin A2 | TGAGAGGAAAACTTC |
| | (CCNA2), RefSeqGene on chromosome 4 [symbol] | AGCTTGTGGGCACTG |
| | | CTGCTATGCTGTTAG |
| SEQ ID | GI 224809230 NG_011435 Homo sapiens | GATGCCCTGTGACCC |
| NO: 2602 | alcohol dehydrogenase 1B | CTAGAGAGTGACAGG |
| | (class 1), beta polypeptide | GTTACCTTTGGACAT |
| | (ADHIB), RefSeqGene on chromosome [symbol] | TCCAGATGTTGCTAC |
| SEQ ID | GI 1007399066 NG 029521 Homo sapiens | TCCAAAACAGTAGGA |
| NO: 2603 | SGT1 homolog, MIS12 kinetochore | ATTCAGCTCCTTCCT |
| | complex assembly cochaperone | TCTCGTCCTTTGTTG |
| | (SUGT1), RefSeqGene c [symbol] | TGGGGTGTAGGGGAG |
| SEQ ID | GI 308080224 NG_027757 Homo sapiens | ATTTGGGGTAACCTC |
| NO: 2604 | transforming growth factor beta receptor 3 | AGAGTAGATGTGGTG |
| | (TGFBR3), RefSeqGene on chromosome 1 | TTGGGGTCAGGTGTG |
| | | GAATGAATGTGGTCT |
| SEQ ID | GI 554506569 NM_001114 Homo sapiens | TTCATAACCGGAAGA |
| NO: 2605 | adenylate cyclase 7 | CTTAACCGGTGGCCT |
| | (ADCY7), transcript variant 1, mRNA | CATCACCAGAGCATC |
| | | GCCAGGATTTCTAAT |
| SEQ ID | GI 169646347 NM_001115152 Homo sapiens | CTCATCCTCCCAGGT |
| NO: 2606 | CD300 molecule like family member d | TACTCCATTGCCGCT |
| | (CD300LD), mRNA | AAAATCACTGGTCCA |
| | | ACAACAGTGAATGGC |
| SEQ ID | GI 50659096 NM_005218 Homo sapiens | GAGGTTGTGCAATCC |
| NO: 2607 | defensin beta 1 | ACCAGTCTTATAAAT |
| | (DEFB1), mRNA | ACAGTGACGCTCCAG |
| | | CCTCTGGAAGCCTCT |
| SEQ ID | GI 182995 M68891 Human GATA-binding protein | AAGAAGGAAGGGATC |
| NO: 2608 | (GATA2) mRNA, complete cds | CAGACTCGGAACCGG |
| | | AAGATGTCCAACAAG |
| | | TCCAAGAAGAGCAAG |
| SEQ ID | GI 1139695586 NG 052809 Homo sapiens | GATTGTTGCCCTGTG |
| NO: 2609 | B-cell CLL/lymphoma 3 | ACTGCCTCACATGGC |
| | (BCL3), RefSeqGene on chromosome 19 | GTGAGACAGTTTTAC |
| | | TTAGTGGTTCCATGG |
| SEQ ID | GI 221554534 NG_009447 Homo sapiens | TCTCCTATGGTTTCC |
| NO: 2610 | dual oxidase 2 | TCTCATCCCTCGGCA |
| | (DUOX2), RefSeqGene on chromosome 15 [symbol] | CCTCTTGTTTTCCTT |
| | | TTTCCTGGGTTCCTT |
| SEQ ID | GI 723428434 NG_041793 Homo sapiens | TGTATGTTCTCTGTT |
| NO: 2611 | inhibitor of nuclear factor | GGGAACCCCTCTGTT |
| | kappa B kinase subunit beta | CACAGCCTCTTAAAA |
| | (IKBKB), RefSeqGene on chromosom [symbol] | GAAGCAGCTGGGCTA |
| SEQ ID | GI 1026191091 NG_047061 Homo sapiens | TGGTGTCTGGCATAG |
| NO: 2612 | marker of proliferation Ki-67 | GGGGTGTGATCAATA |
| | (MKI67), RefSeqGene on chromosome 10 [symbol] | CCTAGCATTCTGGCA |
| | | AGGGTCAGTAGAAAC |
| SEQ ID | GI 238018085 NG_012206 Homo sapiens | TCAGGTGCAGCAACC |
| NO: 2613 | protein kinase C alpha | TGACTGATGTTTTTA |
| | (PRKCA), RefSeqGene on chromosome 17 [symbol] | CAAATCACCTTTTGG |
| | | TGGCCTCCTGTCACT |
| SEQ ID | GI 584277093 NM_001675 Homo sapiens | GATTCCCTGGCCAAG |
| NO: 2614 | activating transcription factor 4 | GAGATCCAGTACCTG |
| | (ATF4), transcript variant 1, mRNA [symbol] | AAAGATTTGATAGAA |
| | | GAGGTCCGCAAGGCA |
| SEQ ID | GI 47777672 AY623118 Homo sapiens | GGGGTGGAAGGAAAG |
| NO: 2615 | hexokinase 2 | GGGGGGTGATAGAGA |
| | (HK2) gene, complete cds [symbol] | ACTCTTTTGTCCCCA |
| | | CAGTTATGCTAATGA |
| SEQ ID | GI 62632868 AY995114 Homo sapiens | GAGCTACCTTTTCCT |
| NO: 2616 | tumor necrosis factor receptor 1- | TCCAGTTCAGCCCCA |
| | associated death domain protein | AGGGAAGGGAAAGTA |
| | (TRADD) gene, complete cds [symbol] | GTGCCTCTTTCCTAT |
| SEQ ID | GI 294610641 NG_017089 Homo sapiens | AAGTGCCCTGAGGCC |
| NO: 2617 | galectin 3 | TGACTCAACTCTTCA |
| | (LGALS3), RefSeqGene on chromosome 14 | CTTGTCACCCAGATG |
| | | TGTTAACACCATGTA |
| SEQ ID | GI 340805855 NG_029505 Homo sapiens | CTGTTGCTTCCTTTC |
| NO: 2618 | methyl-CpG binding domain protein 1 | CACTTTTCTTCTTCC |
| | (MBD1), RefSeqGene on chromosome 18 | CTCTTCCTCCCCACA |
| | | GCACAGAGAATTGCC |
| SEQ ID | GI 162329557 NG_007161 Homo sapiens | TACTGCTACGGAGGA |
| NO: 2619 | MYC proto-oncogene, bHLH transcription factor | GCAGCAGAGAAAGGG |
| | (MYC), RefSeqGene on chromosome 8 | AGAGGGTTTGAGAGG |
| | | GAGCAAAAGAAAATG |
| SEQ ID | GI 209447065 NG_008724 Homo sapiens | GCCCAGTGGTTTCAA |
| NO: 2620 | NLR family apoptosis inhibitory protein | GAGGCAAAGAATCTG |
| | (N AIP), RefSeqGene on chromosome 5 | AATGAGCAGCTGAGA |
| | | GCAGCTTATACCAGC |
| SEQ ID | GI 195222745 NM_018290 Homo sapiens | TGACCTTGTTCCTCC |
| NO: 2621 | phosphoglucomutase 2 | TGAGGCTGTTCCTGA |
| | (PGM2), mRNA | ACAGAAAGATCCGGA |
| | | TCCTGAGTTTCCAAC |
| SEQ ID | GI 1015637181 AH000040 Homo sapiens | GTAATCAGCACAGTG |
| NO: 2622 | hematopoietic stem cell antigen | TTCACCACCCCAGCC |
| | (CD34) gene, complete cds | AACGTTTCAACTCCA |
| | | GAGACAACCTTGAAG |
| SEQ ID | GI 114731556 DQ866809 Homo sapiens | ACACACTGACAAATC |
| NO: 2623 | endoglin | AAAACAGCACACCTG |
| | (Osler-Rendu-Weber syndrome 1) | GGCATGTATGGTCTG |
| | (ENG) gene, complete cds [symbol] | GTTCTCCAAGGACAC |
| SEQ ID | GI 357197116 NG_030591 Homo sapiens | TCAATGCTTCAGCCC |
| NO: 2627 | cholecystokinin B receptor | CTATAAACTCAGGGC |
| | (CCKBR), RefSeqGene on chromosome 11 [symbol] | TGGCTGAACCAGGTC |
| | | CTCTCCAAAAGATTA |
| SEQ ID | GI 315013554 NG_027996 Homo sapiens | TCCCTCTCCTTCCCC |
| NO: 2628 | granzyme A | ACTACGTTCCTCTGC |
| | (GZMA), RefSeqGene on chromosome 5 [symbol] | CATTTAACATCGGCA |
| | | TGTCTGAAATGTCTG |
| SEQ ID | GI 237858685 NG_012086 Homo sapiens | TTGGGGAAGGGTCAC |
| NO: 2629 | interleukin 4 receptor | TTCTAAGCTCATACA |
| | (IL4R), RefSeqGene on chromosome 16 [symbol] | ATATTGGTGACATTC |
| | | AGTCCCTGGCAGGCT |
| SEQ ID | GI 227908850 NG_011958 Homo sapiens | CCGAGAAGGAATCTC |
| NO: 2630 | noggin | AGGAAGTTTGTTTAC |
| | (NOG), RefSeqGene on chromosome 17 [symbol] | ACTGGGGGCCTGGAG |
| | | ACCTTCGGGAAAATA |
| SEQ ID | GI 241896851 NG_012345 Homo sapiens | CAGTCCTTCTCATCT |
| NO: 2631 | peroxisome proliferator activated receptor delta | CACCCAGGATCACCT |
| | (PPARD), RefSeqGene onchromosome 6 [symbol] | GGGAGTGACCAAAGT |
| | | AGGCCAAATGTGATT |
| SEQ ID | GI 343432629 NG_029716 Homo sapiens | CCCATCATGGTGCCA |
| NO: 2632 | transient receptor potential | TTTAGGAAGCATCTG |
| | cation channel subfamily V member 1 | AGTGCTCACCATTTA |
| | (TRPV1),RefSeqGene on [symbol] | GCCACATCGAGCATT |
| SEQ ID | GI 1151099595 NG_052968 Homo sapiens | AAGACTCTGAAACGA |
| NO: 2633 | ubiquitin like modifier activating enzyme 5 | GAAGGTGTTTGTGCA |
| | (UBA5), RefSeqGene on chromosome 3 [symbol] | GCCAGTCTTCCTACC |
| | | ACTATGGGTGTGGTT |
| SEQ ID | GI 308522770 NM_005469 Homo sapiens | ATTGACCAGTATTTA |
| NO: 2634 | acyl-CoA thioesterase 8 | AGGGACCCTAACCTC |
| | (ACOT8), mRNA [symbol] | CAAAAGAGGTACCCA |
| | | TTGGCGCTCAACCGA |
| SEQ ID | GI 156546893 NM_001101802 Homo sapiens | GTTTTGGGAGTCAGC |
| NO: 2635 | PHD finger protein 21A | TAGTATGGAGTACGA |
| | (PHF21A), transcript variant 1, mRNA [symbol] | GATGCAACCCAGCCC |
| | | CCAAACCTACATTCT |
| SEQ ID | GI 306140488 NM_030956 Homo sapiens | ACTTCTGCTGTCTCC |
| NO: 2636 | toll like receptor 10 | ACTTTGATCTGCCCT |
| | (TLR10), transcript variant 1, mRN [symbol] | GGTATCTCAGGATGC |
| | | TAGGTCAATGCACAC |
| | | A |
| SEQ ID | GI 164695782 AK315592 Homo sapiens | CAGGCGTGGACTCTT |
| NO: 2637 | cDNA, FLI96670, Homo sapiens | TCTATCTCGAGATCT |
| | toll interacting protein | TCGATGAGAGAGCCT |
| | (TOLLIP), mRNA [symbol] | TCTCCATGGACGACC |
| SEQ ID | GI 90657251 DQ451402 Homo sapiens | CAGCCGCTAGCATCC |
| NO: 2638 | heat shock 70 kDa protein 1A | TAGAAGTAATTCCCT |
| | (HSPA1A) gene, complete cds [symbol] | CCTCCTTGGAAAACG |
| | | CCCACTACAATCCTT |
| SEQ ID | GI 338221701 NG_029188 Homo sapiens | CTGGGCCTTAGACTC |
| NO: 2639 | caspase 9 | CAGAGTACTGCATGG |
| | (CASP9), RefSeqGene on chromosome 1 [symbol] | GAATGTTTCCCAAGG |
| | | TAGGTTTCATGGTGA |
| SEQ ID | GI 307746866 NG_027761 Homo sapiens | TCAATGTTGCCGCAG |
| NO: 2640 | heat shock protein family A | GATTACAAGCCCTGC |
| | (Hsp70) member 5 | TGAAAGAGATGCATG |
| | (HSPA5), RefSeqGene on chromosome 9 [symbol] | ATGCAACCTCTGTAG |
| SEQ ID | GI 939106626 NG_029107 Homo sapiens | TCTTAAAGACACGGT |
| NO: 2641 | integrin subunit alpha 3 | TGTGGTCAGCGGCTT |
| | (ITGA3), RefSeqGene on chromosome 17 | CAGTCCTAGCTCTGC |
| | | CTCCCATAAAATTCA |
| SEQ ID | GI 207444685 NG_008651 Homo sapiens | ACCCGTGATTCTGAA |
| NO: 2642 | NLRfamily pyrin domain containing 12 | TCAGCAGGTCTTAGG |
| | (NLRP12), RefSeqGene on chromosome 19 | CAACAGGAATCTGCA |
| | | TTTTGATGTGGCTGG |
| SEQ ID | GI 293597503 NG_016969 Homo sapiens | TTCCAAGAACATGTC |
| NO: 2643 | solute carrier family 6 member 2 | CCAAGCAGACAAGCC |
| | (SLC6A2), RefSeqGene on chromosome 16 | CCAGTGATGTACAAG |
| | | TAGCTCTTCCAGTCA |
| SEQ ID | GI 196259816 NG_008365 Homo sapiens | CTCTCCACTAGGGCT |
| NO: 2644 | steroid 5 alpha-reductase 2 | TCTCTGATACTACCC |
| | (SRD5A2), RefSeqGene on chromosome 2 | AGGCCAGCAGGAGGA |
| | | TGTGGAGTTAAGAAA |
| SEQ ID | GI 42794755 NM_016234 Homo sapiens | TCCAAGGGGATATTC |
| NO: 2645 | acyl-CoA synthetase long chain family member 5 | GGTTGCTGGCTGACG |
| | (ACSL5), transcript variant 1, mRNA | ACATGAAGACTTTGA |
| | | AGCCCACATTGTTTC |
| SEQ ID | GI 172072641 NM_005373 Homo sapiens | AACAAATCCAGGACT |
| NO: 2646 | MPL proto-oncogene, thrombopoietin receptor | ACAGACCCCACAGTT |
| | (MPL), mRNA | CTCTCGCTGCCACTT |
| | | CAAGTCACGAAATGA |
| SEQ ID | GI 633257761 NM_001143836 Homo sapiens | CCACCAGATGTTGGG |
| NO: 2647 | NADPH oxidase 4 | GCTAGGATTGTGTCT |
| | (NOX4), transcript variant NOX4B, mRNA | AAGCAGAGCCTCAGC |
| | | ATCTGTTCTTAACCT |
| SEQ ID | GI 756761760 NM_152901 Homo sapiens | CGCCTCCTCCAGCCC |
| NO: 2648 | pyrin domain containing 1 | CTGCACCTGTCATTT |
| | (PYDC1), mRNA | ATTCTTCCACTGCCC |
| | | AATAAATATTCATGG |
| SEQ ID | GI 1036029697 AH005415 Homo sapiens | TTGCGTCCCGCTGAG |
| NO: 2649 | bradykinin B1 receptor | ATGTCACTAGCAGAA |
| | (BDKRB1) gene, complete cds | AAAGGGAACACAAGC |
| | | AAAAGGCAAGATTCT |
| SEQ ID | GI 117606559 EF064754 Homo sapiens | TTCACCCTGGAGCCC |
| NO: 2650 | CD40 molecule, TNF receptor | ATCCAGTCTCCCAAC |
| | superfamily member 5 | TTGTATTAAAGACAG |
| | (CD40) gene, complete cds | AGGCAGAAGTTTGGT |
| SEQ ID | GI 117606557 EF064753 Homo sapiens | TGGAAGACAGCAATC |
| NO: 2651 | CD40 ligand | ATGGTGGCCAAGCCT |
| | (TNF superfamily, member 5, | TGACTGAGCTTTTTC |
| | hyper-IgM syndrome) | TTCTCCAGGCTAAAG |
| | (CD40LG) gene, complete cds [symbol] | |
| SEQ ID | GI 20160210 AY094608 Homo sapiens | CATTCTAGACACCCT |
| NO: 2652 | cyclin-dependent kinase inhibitor 2C | CCCCAACCCCACATC |
| | (CDKN2C) gene, complete cds | CCCAAAATCCAAGCC |
| | | ACTTCTAAGTAGTGA |
| SEQ ID | GI 237820664 NG_012051 Homo sapiens | GTCCTCACACTGGGT |
| NO: 2653 | caveolin 1 | GAGCAGCTTATGGTG |
| | (CAV1), RefSeqGene on chromosome 7 | ATTCCAGACATGATC |
| | | TCTGTTGGGAGTGAC |
| SEQ ID | GI 315360648 NG_028024 Homo sapiens | AGAAGAGCATTCCTG |
| NO: 2654 | glucokinase regulator | TTAGGTCATTCACCA |
| | (GCKR), RefSeqGene on chromosome 2 | AGTCCCAGAAGGGGG |
| | | ACTGGGCATGATATG |
| SEQ ID | GI 302058260 NG_023447 Homo sapiens | ACCATGCCTAGCAAG |
| NO: 2655 | resistin | AGGCATTAATTTTGT |
| | (RETN), RefSeqGene on chromosome 19 | CATGTTTGCATCAGC |
| | | CACCCTTGCATGCAA |
| SEQ ID | GI 45243468 NM_002934 Homo sapiens | AGAGATCAACGACGA |
| NO: 2656 | ribonuclease A family member 2 | GACCCTCCACAGTAT |
| | (RNASE2), mRNA | CCGGTGGTTCCAGTT |
| | | CACCTGGATAGAATC |
| SEQ ID | GI 24079970 AF550128 Homo sapiens | GTTCTAAGCAGAAGC |
| NO: 2657 | excision repair cross- | CCTGTTGCCTTTCTG |
| | complementing rodent repair deficiency, | ACCTAGCTTTGGAAG |
| | complementation group 5 | TTGTGCAGCATCCTT |
| | (ERCC [symbol] | |
| SEQ ID | GI 270047497 NG_016131 Homo sapiens | CCAGGGAGAATAAAG |
| NO: 2658 | connective tissue growth factor | CCTATTCTTTCCTTG |
| | (CTGF), RefSeqGene on chromosome 6 | TCCTGACCTCACTCC |
| | | CTCAGGCTGCATGTT |
| SEQ ID | GI 226492969 NG_011817 Homo sapiens | GAGTGGATTGGTGGG |
| NO: 2659 | LIF receptor alpha | GAGCAAGACTGGAAA |
| | (LIFR), RefSeqGene on chromosome 5 | CAAGAGGACCACTTC |
| | | AGAGGCTCTTATAGC |
| SEQ ID | GI 608785172 KJ534783 Homo sapiens | GAATCCAGAAGCAGA |
| NO: 2660 | clone BZRAPl_iso-A_ | GCTTCCGCTGACAGC |
| | adult-A09 benzodiazapine | TGGCGAGTACATCTA |
| | receptor peripheral- | CATCTATGGCAACAT |
| | associated protein | |
| | 1 isofo [symbol] | |
| SEQ ID | GI 1145587777 KX533498 Homo sapiens | CTACAGTGGTACCTG |
| NO: 2661 | macrophage receptor | GGGGACAATTTGCGA |
| | (MARCO) mRNA, complete cds | TGACGAGTGGCAAAA |
| | | TTCTGATGCCATTGT |
| SEQ ID | GI 22859017 AY148100 Homo sapiens | GGAGCAAGCCGCTGG |
| NO: 2662 | colony stimulating factor 3 receptor | ATATCTGGAGGGAAA |
| | (granulocyte) | ACATTTCAAGCAGAA |
| | (CSF3R) gene, complete cds | GTAACAACCAGTGCA |
| SEQ ID | GI 363807289 NG_031933 Homo sapiens | TGGTGTTACAACAAC |
| NO: 2663 | BMP binding endothelial regulator | TGGACCTCCAATGGG |
| | (BMPER), RefSeqGene on chromosome 7 | GTGATTAGTCCTTTA |
| | | CCTTGGCGTCACTCA |
| SEQ ID | GI 225735577 NG_011719 Homo sapiens | GATGGTTTTCTGGTG |
| NO: 2664 | integrin subunit alpha M | TCCCTTTAGGTCCCA |
| | (ITGAM), RefSeqGene on chromosome 16 | GCCAGTACTGAGAGT |
| | | CAAGGCAATCATGGA |
| SEQ ID | GI 238018085 NG_012206 Homo sapiens | GTTCCTTGGAGCAAT |
| NO: 2665 | protein kinase C alpha | TCTCAGAGCTAGGCT |
| | (PRKCA), RefSeqGene on chromosome 17 | GCTTAAGGCATATGA |
| | | GGGCTCAGGACTCAG |
| SEQ ID | GI 229577394 NG_011993 Homo sapiens | ACTTAGCACACTAGG |
| NO: 2666 | thymocyte selection associated | AGGTGGAATCCTATG |
| | high mobility group box | CCCAGAGTGATGAGA |
| | (TOX), RefSeqGene on chromosome [symbol] | TGGGGCTAGCTAGTG |
| SEQ ID | GI 356461041 NM_014329 Homo sapiens | CTGCATTGGGATTTT |
| NO: 2667 | enhancer of mRNA decapping 4 | GGCCAAGGAGGTGGA |
| | (EDC4), mRNA | GATTGTGGCTAGCAG |
| | | TGACTCTAGCATTTC |
| SEQ ID | GI 261337164 NM_206943 Homo sapiens | ACCTGCGATTGCTTT |
| NO: 2668 | latent transforming growth | GATGGGTATCACTTG |
| | factor beta binding protein 1 | GATACGGCCAAGATG |
| | (LTBP1), transcript variant 1, mRN [symbol] | ACCTGTGTCGATGTA |
| SEQ ID | GI 4885488 NM_005372 Homo sapiens | GGGCGTGACGCCTAA |
| NO: 2669 | MOS proto-oncogene, serine/ | AGCCGACATTTATTC |
| | threonine kinase | CTTTGCCATCACTCT |
| | (MOS), mRNA | CTGGCAAATGACTAC |
| SEQ ID | GI 166064064 NM_002831 Homo sapiens | CATGACACAACCGAA |
| NO: 2670 | protein tyrosine phosphatase, | TACAAACTCCGTACC |
| | non-receptor type 6 | TTACAGGTCTCCCCG |
| | (PTPN6), transcript variant 1, mRNA | CTGGACAATGGAGAC |
| SEQ ID | GI 81301989 DQ217942 Homo sapiens | TAGAGGAATGAGGTC |
| NO: 2671 | EGF-like module containing | TCCCACTGTCACCAA |
| | mucin-like hormone receptor-like 1 | ACAGAACTAGGTCCA |
| | (EMR1) gene, complete cds | TTTGCCCATGTGCAA |
| SEQ ID | GI 219521817 NG_009217 Homo sapiens | GAGTGATTGAGATTT |
| NO: 2672 | cathepsin B | GCGTTGTTGTGGAGA |
| | (CTSB), RefSeqGene on chromosome 8 | GAAAGGCGCCTGTTG |
| | | CACAATGGAGTGAGA |
| SEQ ID | GI 219689119 NG_009261 Homo sapiens | GCAGAGGAAATGTCA |
| NO: 2673 | elastin | ACCCACCTGCAATCC |
| | (ELN), RefSeqGene on chromosome 7 | TGCATTCAGGACCAA |
| | | CTGTCACTTCCATAC |
| SEQ ID | GI 353409895 NG_030365 Homo sapiens | TTCGACTCCGGGTGT |
| NO: 2674 | POU class 2 homeobox 2 | GTGAGTTTGTATACT |
| | (POU2F2), RefSeqGene on chromosome 19 | TTTTACGTGTGTCTG |
| | | GCTGGCTGTGTGTCA |
| SEQ ID | GI 1069415403 NG_032899 Homo sapiens | CAGAGATTACCAAGC |
| NO: 2675 | protein C receptor | ATGGTTCCATAGCCT |
| | (PROCR), RefSeqGene on chromosome 20 | AGAGGAGTCCAGTGT |
| | | GGCCTGTGTGTGTTT |
| SEQ ID | GI 341572562 NG_029524 Homo sapiens | GTAGTCCCCCCATGT |
| NO: 2676 | TNF superfamily member 13b | GCCTACTCACTGATG |
| | (TNFSF13B), RefSeqGene on chromosome 13 | GCTACTGTCTTCTCT |
| | | CTGTCCCTTTCATCT |
| SEQ ID | GI 344925862 NG_029825 Homo sapiens | GGGTGGGTATTCTGT |
| NO: 2677 | ubiquitin specific peptidase 2 | TGTGGGCAAGCTTTA |
| | (USP2), RefSeqGene on chromosome 11 | ACATTTAGTTCCGAG |
| | | TGCGCAACATGGAGA |
| SEQ ID | GI 209447082 NG_008731 Homo sapiens | GGCACCTCTTGTCGG |
| NO: 2678 | vitamin D receptor | GTCATCACGATCTGT |
| | (VDR), RefSeqGene on chromosome 12 | ACCCATATCTTCACC |
| | | CAAGGACTGTTTGTC |
| SEQ ID | GI 254039651 NG_012413 Homo sapiens | GTGGCTGCCAAGAAC |
| NO: 2679 | vimentin | CTGCAGGAGGCAGAA |
| | (VIM), RefSeqGene on chromosome 10 | GAATGGTACAAATCC |
| | | AAGGTAGGAAACAAA |
| SEQ ID | GI 928192547 NM_006472 Homo sapiens | GAGTACCTGCGCTAT |
| NO: 2680 | thioredoxin interacting protein | GAAGACACGCTTCTT |
| | (TXNIP), transcript variant 1, mRNA | CTGGAAGACCAGCCA |
| | | ACAGGTGAGAATGAG |
| SEQ ID | GI 94717603 DQ508420 Homo sapiens | ATGTGTCTTATTAGC |
| NO: 2681 | caspase 5, apoptosis- | CAGCAATGTGGCTCA |
| | related cysteine peptidase | GGCTCGTCCTCATTT |
| | (CASP5) gene, complete cds | AGGTTCAGGTGTCCA |
| SEQ ID | GI 21885466 AY128534 Homo sapiens | AAATGCCAAAAGTGC |
| NO: 2682 | cyclin-dependent kinase 6 | CCAGATTGAGAAACC |
| | (CDK6) gene, complete cds | CCTTAACATAGTTAT |
| | | GCCTGGCTACCCCCG |
| SEQ ID | GI 45238842 AY563103 Homo sapiens | GTACAGGAAACTCTA |
| NO: 2683 | interleukin 2 receptor, alpha | GCCACTCGTCCTGGG |
| | (IL2RA) gene, complete cds | ACAACCAATGTCAAT |
| | | GCACAAGCTCTGGTA |
| SEQ ID | GI 198442852 NG_008474 Homo sapiens | GGAAGGGCTTTTGGG |
| NO: 2684 | aryl hydrocarbon receptor | AATTTTCTGGGGCCC |
| | interacting protein like 1 | TAAATGTTGAGTGAG |
| | (AIPL1), RefSeqGene on chromosome 17 [symbol] | AAGAAAGGGAGTCCG |
| SEQ ID | GI 297307113 NG_021395 Homo sapiens | CAGGAGCATGGCCTA |
| NO: 2685 | erythropoietin receptor | GCACTGTGCAAACAC |
| | (EPOR), RefSeqGene on chromosome 19 | TCAACTGCTGGTCGT |
| | | TGCAAGGATTATTAT |
| SEQ ID | GI 374349247 NG_032044 Homo sapiens | GGACTTCTGAACAGA |
| NO: 2686 | glutamic-oxaloacetic transaminase 1 | CAGTCATAAGGAAGG |
| | (GOT1), RefSeqGene on chromosome 10 | CAGGAGGCAGTGAGA |
| | | ACACCAGGCTCTTTC |
| SEQ ID | GI 226493744 NG_011821 Homo sapiens | CCCAACCTAAACTGA |
| NO: 2687 | nuclear receptor subfamily | ATGTGTTTTTAATGG |
| | 4 group A member 2 | CAATGAGGGAGGGGG |
| | (NR4A2), RefSeqGene on chromosome 2 | AAGCGTGCAGAGTGA |
| SEQ ID | GI 1154345445 NG_033859 Homo sapiens | TGATGCCCTGCTTTG |
| NO: 2688 | sodium voltage-gated | GTTTTGTCTATGCCA |
| | channel alpha subunit 11 | AAAGTTCCTGCTATA |
| | (SCN11A), RefSeqGene on chromosome 3 | CCCACAGTGGTGGTC |
| SEQ ID | GI 442092991JQ924061 Homo sapiens | CTGTTTCGTCCTGTG |
| NO: 2689 | ATG5 | GCTGCAGATGGACAG |
| | (ATG5) mRNA, complete cds | TTGCACACACTAGGA |
| | | GATCTCCTCAAAGAA |
| SEQ ID | GI 323362950 NM_138621 Homo sapiens | CTTGGGCTTTGCCGC |
| NO: 2690 | BCL2 like 11 | CCTTATGATGAAGTG |
| | (BCL2L11), transcript variant 1, mRNA | TGTGTTTGATGCCAG |
| | | TGAGAAACTCAGTCT |
| SEQ ID | GI 23110953 NM_001911 Homo sapiens | TGCGAGAAGACTTTG |
| NO: 2691 | cathepsin G | TGCTGACAGCAGCTC |
| | (CTSG), mRNA | ATTGCTGGGGAAGCA |
| | | ATATAAATGTCACCC |
| SEQ ID | GI 164696344 AK313967 Homo sapiens | GACACCAAAAGCTTC |
| NO: 2692 | cDNA, FLI94614, Homo sapiens | ATCTTGGTGAGAAAA |
| | interleukin 18 receptor 1 | GCAGACATGGCTGAT |
| | (IL18R1), mRNA | ATCCCAGGCCACGTC |
| SEQ ID | GI 164690417 AK312618 Homo sapiens | ACCACAAAATCCAGG |
| NO: 2693 | cDNA, FLI93000, Homo sapiens | AGCAAATTCTGCCAG |
| | RAB5A, member RAS oncogene family | AGGAAGAGGAGTAGA |
| | (RAB5A), mRNA | CCTTACCGAACCCAC |
| SEQ ID | GI 40288424 AY506357 Homo sapiens | GCTGCGCCCCAAGTC |
| NO: 2694 | truncated epidermal growth factor | AAAGTAATAGAGCCA |
| | (beta-urogastrone) | CAGTTTCACCATCAG |
| | (EGF) gene, complete cds | GCCATTTTCTGATGA |
| SEQ ID | GI 1059792660 AH007003 Homo sapiens | AAGCCTGGGAGGTAG |
| NO: 2695 | MET proto-oncogene, intron 2, 3′ end | ATCTCAAAAGTTCTT |
| | | TCTACCCACACTGCT |
| | | TCTCCATCACGCCAC |
| SEQ ID | GI 7417326 AF237581 Homo sapiens | GCAAGAGGAAGGTAT |
| NO: 2696 | migration inhibitory factor- | CATCAGAATTGCCCT |
| | related protein 14 variant P | TAACCAGGAACCAGA |
| | (S100A9) gene, complete cds | ATAGCTGGGTCCCCT |
| SEQ ID | GI 1206876284 NG_033870 Homo sapiens | TCTATGATCCACCAC |
| NO: 2697 | asparagine synthetase | ATTCCGTTTCCCCTG |
| | (glutamine-hydrolyzing) | AAGGGTGATCAAGGA |
| | (ASNS), RefSeqGene on chromosome 7 | CACATGCAGAGATGG |
| SEQ ID | GI 930697465 NM_005564 Homo sapiens | CCTGGATTAACGAGT |
| NO: 2698 | lipocalin 2 | TACCTCGTCCGAGTG |
| | (LCN2), mRNA | GTGAGCACCAACTAC |
| | | AACCAGCATGCTATG |
| SEQ ID | GI 315630394 NM_002611 Homo sapiens | GATGCCTACGACATG |
| NO: 2699 | pyruvate dehydrogenase kinase 2 | GCTAAGCTCCTGTGT |
| | (PDK2), transcript variant 1, mRNA | GACAAGTATTACATG |
| | | GCCTCACCTGACCTG |
| SEQ ID | GI 746815979 NM_032525 Homo sapiens | CCAACTCAGATCCTA |
| NO: 2700 | tubulin beta 6 class V | CAACACGCAAGTTCC |
| | (TUBB6), transcript variant 1, mRNA | TTCTTGAACCCTGGT |
| | | GCCTCCTACCCTATG |
| SEQ ID | GI 62003164 AY974245 Homo sapiens | TTCCACAGGTAGGGG |
| NO: 2701 | alcohol dehydrogenase 4 | TGTCTTCCAGTCCTG |
| | (class II), pi polypeptide | GGTGAAGAAAACATT |
| | (ADH4) gene, complete cds | TATGCATAGCACCCT |
| SEQ ID | GI 88698111 DQ388429 Homo sapiens | GAGAAATTGCAGGTG |
| NO: 2702 | heat shock 70 kDa protein 1B | CCCGTCTTGATTAGA |
| | (HSPA1B) gene, complete cds | GTGGGGCTTGTTTCA |
| | | GGGAAAAGTCGGATG |
| SEQ ID | GI 298676536 NG_021477 Homo sapiens | CCTCCAGTTCAAGGA |
| NO: 2703 | glutamate decarboxylase 1 | CTTTGGGGCCTCTGT |
| | (GAD1), RefSeqGene on chromosome 2 | CTGAGATGCTTAGAA |
| | | ACAGCTCAGACTGTG |
| SEQ ID | GI 237858602 NG_012146 Homo sapiens | ATGAGGCCTGAAGAG |
| NO: 2704 | period circadian clock 2 | AGAGCCTCAGACAAG |
| | (PER2), RefSeqGene on chromosome 2 | AAGGAGGAAATCAAA |
| | | GAATAACGCCAGGCC |
| SEQ ID | GI 14278710 AF374726 Homo sapiens | TTGATGGAGGTGAAA |
| NO: 2705 | coagulation factor 11 receptor-like 2 | ACGGGGGCCTCTGAA |
| | (F2RL2) gene, complete cds | GGAAACAAAGAGGCA |
| | | GTGAATTTGCTTTCT |
| SEQ ID | GI 375331875 NM_000895 Homo sapiens | CCTGAACAGACTTCT |
| NO: 2706 | leukotriene A4 hydrolase | GGGAAGGAACACCCA |
| | (LTA4H), transcript variant 1, mRNA | TATCTCTTTAGTCAG |
| | | TGCCAGGCCATCCAC |
| SEQ ID | GI 187473251 EU600171 Homo sapiens | GAGGGGTTTAAGGCT |
| NO: 2707 | v-raf murine sarcoma viral | CCGCGGGTCATTCTC |
| | oncogene homolog B1 | ATTTACAGCTGTTGC |
| | (BRAF) gene, complete cds | TCAGTGCCATTATTG |
| SEQ ID | GI 14518294 AF390905 Homo sapiens | TTGGCTTAATGCCTG |
| NO: 2708 | interleukin 19 | CCACGTGGTAAGGTC |
| | (IL19) gene, complete cds | TTGACATACACCTGT |
| | | CTATTCCCTGGGAAT |
| SEQ ID | GI 223718000 NG_009785 Homo sapiens | TCCAAAATGGGAACT |
| NO: 2709 | guanidinoacetate N-methyltransferase | AGTGGCCTGTGTTCT |
| | (GAMT), RefSeqGene on chromosome 19 | TTTTCTGGTGTGATT |
| | | CTTTGGCTCCACCCG |
| SEQ ID | GI 1027860277 NG_047185 Homo sapiens | CAGATGCACCTGACT |
| NO: 2710 | NLR family member XI | TGCTGCTATTAAAAA |
| | (NLRX1), RefSeqGene on chromosome 11 | GCCGTGTGCCTTCTA |
| | | CCAATTGTGGCCTCT |
| SEQ ID | GI 345091009 NG_029843 Homo sapiens | TGACCGGCGTGAGTG |
| NO: 2711 | nuclear receptor subfamily 1 | ATAGATGAAGTTAGA |
| | group H member 4 | GCTATGTTGGGATGG |
| | (NR1H4), RefSeqGene on chromosome 12 | GGTAGGATTCAGGGT |
| SEQ ID | GI 1024336776 NG_029865 Homo sapiens | GCCCATTGTCGAAGC |
| NO: 2712 | protein inhibitor of activated STAT 2 | CATAGTTGTGGAAGC |
| | (PIAS2), RefSeqGene on chromosome 18 | AGTCAAAGGTTGGAT |
| | | TTATTCAGGGTGGGT |
| SEQ ID | GI 170014713 NG_007461 Homo sapiens | GAGGCCTAGTGGGAG |
| NO: 2713 | transforming growth factor beta receptor 1 | AAGACTGATTCACTT |
| | (TGFBR1), RefSeqGene on chromosome 9 | GTTCTGGGTTTGAGC |
| | | CCTGTAAACTGCTGT |
| SEQ ID | GI 166064064 NM_002831 Homo sapiens | ATGAGAACCTGCACA |
| NO: 2714 | protein tyrosine phosphatase, | CTAAGAACAAGAGGG |
| | non-receptor type 6 | AGGAGAAAGTGAAGA |
| | (PTPN6), transcript variant 1, mRNA | AGCAGCGGTCAGCAG |
| SEQ ID | GI 28932927 AY245248 Homo sapiens | AGGTCTTTCGGACCC |
| NO: 2715 | BCL2-interacting killer | ATGAGACCCCGTGTC |
| | (apoptosis-inducing) | ATTGCTTTACCTCCC |
| | (BIK) gene, complete cds | TGAGTCTCAATTTTT |
| SEQ ID | GI 350606318 NG_009014 Homo sapiens | TGTCCTCACCACTTA |
| NO: 2716 | androgen receptor | AGTGGGTGTCAAATT |
| | (AR), RefSeqGene on chromosome X | TCCTTCCCTTTCTGC |
| | | ACACGCTGCACAATC |
| SEQ ID | GI 237820672 NG_012057 Homo sapiens | CTTTAGGACAGGAAG |
| NO: 2717 | carboxylesterase 1 | CCATGCATTAGACCG |
| | (CES1), RefSeqGene on chromosome 16 | CAGGAAGAACCCACT |
| | | GAAGTCAGGCTCTAC |
| SEQ ID | GI 226509828 NG_011801 Homo sapiens | ATTGTATGGAAGGAC |
| NO: 2718 | carnitine palmitoyltransferase 1A | CACAGTTTATCCACC |
| | (CPT1A), RefSeqGene on chromosome 11 | TGTTCATCCGCTGTT |
| | | GGGGACGTATCCAGG |
| SEQ ID | GI 1150763505 NG_052952 Homo sapiens | TGGGGTTGCTTTTAG |
| NO: 2719 | ezrin | ATGTTCCAGTGACTG |
| | (EZR), RefSeqGene on chromosome 6 | GGGACACTCAGTCAT |
| | | TGGATGGCTTAAGGA |
| SEQ ID | GI 224809239 NG_011444 Homo sapiens | GTTTCAAGCAGAGGA |
| NO: 2720 | fatty acid binding protein 2 | AACACCCTGGAGCAA |
| | (FABP2), RefSeqGene on chromosome 4 | AGACACCAAGGCAGT |
| | | AGCAAACCTGGTAAA |
| SEQ ID | GI 336285267 NG_029154 Homo sapiens | GAGCTACCACACCCC |
| NO: 2721 | interferon alpha 2 | AGAGGGTGGTATGGT |
| | (IFNA2), RefSeqGene on chromosome 9 | ATGTTGTCTGTTGTG |
| | | AGCTGTGTGAATCAG |
| SEQ ID | GI 1135449810 NG_052673 Homo sapiens | CTGAGACCATCCTGA |
| NO: 2722 | 0-6-methylguanine-DNA methyltransferase | TTGGGATTTGGTCAG |
| | (MGMT), RefSeqGene on chromosome 10 | TGGGAACTCAAGTCA |
| | | GTATTGTCCTGGGGG |
| SEQ ID | GI 237858724 NG_012101 Homo sapiens | CCACGGCTTCACTGG |
| NO: 2723 | matrix metallopeptidase 8 | GTATAATTCTGTGCT |
| | (MMP8), RefSeqGene on chromosome 11 | TAGCGGACGCTGTTC |
| | | CTTCTACTGGATCAC |
| SEQ ID | GI 226061859 NG_011749 Homo sapiens | TCCACAGTGCTCTGT |
| NO: 2724 | peroxisome proliferator | AATCAATTCCAGGAC |
| | activated receptor gamma | CGTGTGGATGGAGAC |
| | (PPARG), RefSeqGene on chromosome 3 [symbol] | CGGAAAGGTTTCTTT |
| SEQ ID | GI 356461041 NM_014329 Homo sapiens | GTGTGCTCATCAAGC |
| NO: 2725 | enhancer of mRNA decapping 4 | TCTTTTGTGTGCATA |
| | (EDC4), mRNA | CTAAGGCACTGCAAG |
| | | ATGTGCAGATCCGCT |
| SEQ ID | GI 115387093 NM_003000 Homo sapiens | CCACACCATCATGAA |
| NO: 2726 | succinate dehydrogenase | CTGCACAAGGACCTG |
| | complex iron sulfur subunit B | TCCTAAGGGTCTGAA |
| | (SDHB), mRNA | TCCAGGGAAAGCTAT |
| SEQ ID | GI 26000792 AY186092 Homo sapiens | ATTTTTTGCTCCTGC |
| NO: 2727 | interleukin-1 receptor- | GAGTCTTTTGCTCCC |
| | associated kinase 4 | AGGTAAACTGATTGT |
| | (IRAK4) gene, complete cds | GACCAGGGTGTCCAC |
| SEQ ID | GI 315360648 NG_028024 Homo sapiens | GGATATGTGTTTAGA |
| NO: 2728 | glucokinase regulator | GGTGAGGATGTGGCC |
| | (GCKR), RefSeqGene on chromosome 2 | CGGATGGAGAATACT |
| | | GAGAGCAGGGAGACT |
| SEQ ID | GI 238776809 NG_012246 Homo sapiens | GCAGTGGCAATGTCC |
| NO: 2729 | N-acetyltransferase 2 | CAGATTTCCCTGGAA |
| | (NAT2), RefSeqGene on chromosome 8 | GTTCAAACCAGGTGT |
| | | TCCACTGAACTTTCT |
| SEQ ID | GI 237874184 NG_012166 Homo sapiens | AGACCCATCACCACT |
| NO: 2730 | T-box 21 | CTCGGTTCCTTCCCA |
| | (TBX21), RefSeqGene on chromosome 17 | GCAAAGTGTCATGTA |
| | | AATCAGGGGCTATTT |
| SEQ ID | GI 378786656 NG_032107 Homo sapiens | TGTGAACTGAGAACC |
| NO: 2731 | TNF receptor superfamily member 10a | TCACTTCTCTGACCT |
| | (TNFRSF10A), RefSeqGene on chromosome 8 | CACCTTCCATCTCCC |
| | | TTAATCAGACGCCCA |
| SEQ ID | GI 238814313 NG_012262 Homo sapiens | TGCCTCATCTTCTGG |
| NO: 2732 | topoisomerase | CCAGGGATTGATCCT |
| | (DNA) 1 | GAGATGGGAAGAGTA |
| | (TOP1), RefSeqGene on chromosome 20 | TGAGAAGAGACACCA |
| SEQ ID | GI 974005372 NM_005944 Homo sapiens | CAGCGAGAACCATGG |
| NO: 2733 | CD200 molecule | GGTGGTGATCCAGCC |
| | (CD200), transcript variant 1, mRNA | TGCCTATAAGGACAA |
| | | GATAAACATTACCCA |
| SEQ ID | GI 57863305 NM_052872 Homo sapiens | AGCILGGAAAATLLL |
| NO: 2734 | interleukin 17F | LAAAGTAGGALATAC |
| | (IL17F), mRNA | TTTTTTCCAAAAGCC |
| | | IGAGAGTTGCCCGCC |
| SEQ ID | GI 149408158 NM_005259 Homo sapiens | CTCCGGGAACTGATT |
| NO: 2735 | myostatin | GATCAGTATGATGTC |
| | (MSTN), mRNA | CAGAGGGATGACAGC |
| | | AGCGATGGCTCTTTG |
| SEQ ID | GI 523498705 NM_024923 Homo sapiens | TAGCATCAGGGTCTA |
| NO: 2736 | nucleoporin 210 | TGATCGGGACATCCA |
| | (NUP210), mRNA | CCATCGAAGTGATTG |
| | | CACAAGAGCCCTTTG |
| SEQ ID | GI 156523967 NM_001618 Homo sapiens | TGACTATGGCCAGGA |
| NO: 2737 | poly(ADP-ribose) polymerase 1 | TGAAGAGGCAGTGAA |
| | (PARP1), mRNA | GAAGCTGACAGTAAA |
| | | TCCTGGCACCAAGTC |
| SEQ ID | GI 315630394 NM_002611 Homo sapiens | CTGATGACCAGGTTC |
| NO: 2738 | pyruvate dehydrogenase kinase 2 | TGTCTCTATGGAAGT |
| | (PDK2), transcript variant 1, mRNA | CACTGCGGTGATAGG |
| | | TCTGTGATGGTCCCT |
| SEQ ID | GI 88698227 DQ400449 Homo sapiens | AGTCTCCATGGCAAC |
| NO: 2739 | complement component 5 | CAGTCATCAAGGCTC |
| | (C5) gene, complete cds | CAGCAACCTTTTCAT |
| | | AGACACTCTCCACTC |
| SEQ ID | GI 40786798 AY518533 Homo sapiens | GTTCTTTTGATATTG |
| NO: 2740 | interleukin 17 receptor B | GACAATGCCCCCAGC |
| | (IL17RB) gene, complete cds | CACCCAGAGCCCCAT |
| | | GAGTTCAACACTAAA |
| SEQ ID | GI 589269250 NG_007475 Homo sapiens | AGCAGGTTGGTGGCA |
| NO: 2741 | aquaporin 1 | GAATTGGGATTTGAA |
| | (Colton blood group) | CCTATTCTGCTTGGC |
| | (AQP1), RefSeqGene on chromosome 7 | TTCAAAGATCAGGCT |
| SEQ ID | GI 259013220 NG_012921 Homo sapiens | CGCACGTCCAAGGAA |
| NO: 2742 | bisphosphoglycerate mutase | AAGCCAGGTACCAAA |
| | (BPGM), RefSeqGene on chromosome 7 | CTGAAACACTGGATG |
| | | TAGTCTGCATCTTGC |
| SEQ ID | GI 347800632 NG_030032 Homo sapiens | CTGCTGCTGCAATTT |
| NO: 2743 | HSPB1 associated protein 1 | CTACCTGCTCTAGGG |
| | (HSPBAP1), RefSeqGene on chromosome 3 | GTTTTGGACAGCTGT |
| | | TTTCTCTGGCAGTTC |
| SEQ ID | GI 1028908919 NG_050629 Homo sapiens | TGTGGGATTTTGTGT |
| NO: 2744 | neurotrophin 3 | TGGTGAGGACTGGAG |
| | (NTF3), RefSeqGene on chromosome 12 | TGTAGCTGGACTTAG |
| | | AGGGTTTGGTTGTGT |
| SEQ ID | GI 374349248 NG_032045 Homo sapiens | AACAGACAGACACTG |
| NO: 2745 | phospholipase A2 group V | GGTTAGAAACTTTTC |
| | (PLA2G5), RefSeqGene on chromosome 1 | GGTGCTTGCACGGGT |
| | | GGATTTCACTTTGCA |
| SEQ ID | GI 153082751 NM_002199 Homo sapiens | CATCCCGCTCTTTTC |
| NO: 2746 | interferon regulatory factor 2 | CTACTTTTCCTTCCC |
| | (IRF2), mRNA | TCAAAGCTTCCATTC |
| | | CACATCCGGAGGAGA |
| SEQ ID | GI 1036030581 AH008227 Homo sapiens | CCATAACCAATGTCA |
| NO: 2747 | chromosome 1 PEA15 protein | GCAACTCAGCTTTGG |
| | (PEA15) gene, complete cds | TTCCAGGTCACTAGT |
| | | CTGGTGGAGTAGGGG |
| SEQ ID | GI 153791376 NM_024310 Homo sapiens | GTCTCATGCCTTTTT |
| NO: 2748 | pleckstrin homology and | GTCCTGTCCTCAGCT |
| | FYVE domain containing 1 | CTCCGTGTGGTCAGC |
| | (PLEKHF1), mRNA | GAAACCATTGTTTTC |
| SEQ ID | GI 45243468 NM_002934 Homo sapiens | AGTTTACCTGGGCTC |
| NO: 2749 | ribonuclease A family member 2 | AATGGTTTGAAACCC |
| | (RNASE2), mRNA | AGCACATCAATATGA |
| | | CCTCCCAGCAATGCA |
| SEQ ID | GI 62003164 AY974245 Homo sapiens | AGTGAGATCACTGAT |
| NO: 2750 | alcohol dehydrogenase 4 | GGCAAGTTTGGACAA |
| | (class II), pi polypeptide | TTTCCCCATTTTTCC |
| | (ADH4) gene, complete cds | CAAGCTGACCAGGCT |
| SEQ ID | GI 1049010865 AH002678 Homo sapiens | AGTCTCTCTCTTATA |
| NO: 2751 | isolate Patient with P-450(C21) | AAGCCCTCCTTTTTC |
| | deficiency membrane protein | ATCCTAGCATTGGGA |
| | (CD3E) gene, complete cds | GCAATGGCCCCAGGG |
| SEQ ID | GI 2789671 AF040714 Homo sapiens | GCACAAGAAATGTCA |
| NO: 2752 | homeobox protein A10 | GCCAGAAAGGGCTAT |
| | (HOXA10) gene, complete cds | CTGCTCCCTTCGCCA |
| | | AATTATCCCACAACA |
| SEQ ID | GI 117606495 EF064722 Homo sapiens | CTTCTCAAGATCCGA |
| NO: 2753 | interleukin 6 signal transducer | GTCTACCCAGCCCTT |
| | (gpl30, oncostatin M receptor) | GTTAGATTCAGAGGA |
| | (II6ST) gene, complete cds | GCGGCCAGAAGATCT |
| SEQ ID | GI 1024846743 NG_047006 Homo sapiens | CAGCTGGGTGCATAG |
| NO: 2754 | hexokinase 3 | TGAGGGCTGGAGCAA |
| | (HK3), RefSeqGene on chromosome 5 | TGTTTTGGGAAGGAT |
| | | GACTGACTGAAAAAA |
| SEQ ID | GI 1028224162 NG_047195 Homo sapiens | GAACACCTTGGAACC |
| NO: 2755 | transducin beta | AGGGATCCCATAGTG |
| | like 1 X-linked receptor 1 | GAATCTCAGTGGGGC |
| | (TBL1XR1), RefSeqGene on chromosome 3 | TTATATTCCTGCTGG |
| SEQ ID | GI 371877908 NG_031992 Homo sapiens | ACTTGTATCAGGACC |
| NO: 2756 | Wnt family member 5A | ACATGCAGTACATCG |
| | (WNT5A), RefSeqGene on chromosome 3 | GAGAAGGCGCGAAGA |
| | | CAGGCATCAAAGAAT |
| SEQ ID | GI 125661056 NM_005195 Homo sapiens | GACATAGGAGCGCAA |
| NO: 2757 | CCAAT/enhancer binding protein delta | AGAAGCTACAGCCTG |
| | (CEBPD), mRNA | GACTTACCACCACTA |
| | | AACTGCGAGAGAAGC |
| SEQ ID | GI 82659110 NM_007172 Homo sapiens | AGGTCTTTTCCAAGC |
| NO: 2758 | nucleoporin 50 | AGGCACTCAGAACAC |
| | (NUP50), transcript variant 2, mRNA | AGGTCACCGTATGTT |
| | | CTCAGCCAGTAACAA |
| SEQ ID | GI 167234418 NM_005119 Homo sapiens | AGGCCCTATTATTTC |
| NO: 2759 | thyroid hormone receptor | CGTGGGCGTAACAGA |
| | associated protein 3 | GGCTTTTATCCATGG |
| | (THRAP3), mRNA | GGCCAATATAACCGA |
| SEQ ID | GI 81301989 DQ217942 Homo sapiens | TTTGTTGGGACAGGG |
| NO: 2760 | EGF-like module containing | TTAACTGTAAGGTCA |
| | mucin-like hormone receptor-like 1 | GGACTCTGAGACAAG |
| | (EMR1) gene, complete cds | CGCTGACTCCTCCTA |
| SEQ ID | GI 300116194 NG_023256 Homo sapiens | ACGTGAGCTCTCTGG |
| NO: 2761 | linker for activation of | CCTCAGTTTCTCGAT |
| | T-cells family member 2 | TTGTGAAATGCAGTG |
| | (LAT2), RefSeqGene on chromosome 7 | AACCTCGTCCTCAAA |
| SEQ ID | GI 238814317 NG_012266 Homo sapiens | GGGACGGAGGGTTGA |
| NO: 2762 | X-box binding protein 1 | TGTCATAGCAAGATT |
| | (XBP1), RefSeqGene on chromosome 22 | GCAGCCTTCACTAGA |
| | | TTAATGAGGCCAGGT |
| SEQ ID | GI 229608964 NM_014600 Homo sapiens | TTTTGCAGTTGAGGA |
| NO: 2763 | EH domain containing 3 | GGCTGAGTAGCCTCA |
| | (EHD3), mRNA | GAAGGGTTTAGGCGA |
| | | CCTTCTGAAACTCAC |
| SEQ ID | GI 213972644 NM_002748 Homo sapiens | TTTACATTGTTCAGG |
| NO: 2764 | mitogen-activated protein kinase 6 | AGTACATGGAGACAG |
| | (MAPK6), mRNA | ACTTGGCTAATGTGC |
| | | TGGAGCAGGGCCCTT |
| SEQ ID | GI 1147566 X80590 H. sapiens | CTAGGGGGCAGGTGG |
| NO: 2765 | PHKGI mRNA | GGAGGGGAAGCCATG |
| | | GAAATACAAGTCAAA |
| | | GGGGTAAAAAAAAAA |
| SEQ ID | GI 1049010946 AH002554 Homo sapiens | ATAGAATCCTGTGAA |
| NO: 2766 | actin prepeptide | GCAGCTCCAGCTATG |
| | (ACTA2) gene, complete cds | TGTGAAGAAGAGGAC |
| | | AGCACTGCCTTGGTG |
| SEQ ID | GI 255652911 NG_012637 Homo sapiens | TCAGCTGAGCTAAGG |
| NO: 2767 | C-C motif chemokine receptor 5 | GGCTCCCACCAGGTT |
| | (gene/pseudogene) | TGAGCCTATCTAATG |
| | (CCR5), RefSeqGene on chromosome 3 | AATCCATGAGGTAGA |
| SEQ ID | GI 255652920 NG_012644 Homo sapiens | GTGAGACCCAGAAGA |
| NO: 2768 | deleted in malignant brain tumors 1 | CTGTGGCCAAAGCTA |
| | (DMBT1), RefSeqGene on chromosome 10 | TTATGGACCCATGGT |
| | | CTCCGTGGAATCATC |
| SEQ ID | GI 336285218 NG_029143 Homo sapiens | TTGATGGAGGACTTT |
| NO: 2769 | mitogen-activated protein kinase kinase 5 | GAGGTGAGCTTCATG |
| | (MAP2K5), RefSeqGene on chromosome 15 | GAGACAATGGTGGCA |
| | | TTCAAGCTGGGTCTA |
| SEQ ID | GI 1024357482 NG_046924 Homo sapiens | CCTTAGGCCGTCCAG |
| NO: 2770 | NLR family pyrin domain containing 5 | TCATCTTTCTCTGGG |
| | (NLRP5), RefSeqGene on chromosome 19 | GCTTGATTGATCAGT |
| | | TCCCACTCTGACAAC |
| SEQ ID | GI 1147592400 NG_029037 Homo sapiens | AGATTCCACCACCAA |
| NO: 2771 | reticulon 4 | ACCTCTGTGACCACA |
| | (RTN4), RefSeqGene on chromosome 2 | ACCTCCTCTATGTTC |
| | | ACTACAGCCACTTGC |
| SEQ ID | GI 257467583 NG_012852 Homo sapiens | ACCCCCGAGCAGATC |
| NO: 2772 | signal transducer and activator | TTTACCAGGCCTGTA |
| | of transcription 4 | CATGGATCCCCAACT |
| | (STAT4), RefSeqGene on chromosome 2 | GTTTTGAGTATTGAC |
| SEQ ID | GI 674651085 NG_012882 Homo sapiens | CCTAGTTCTGCCTGC |
| NO: 2773 | toll like receptor 8 | AATGGCACCAGCTCA |
| | (TLR8), RefSeqGene on chromosome X | ACCACCTGGTACATA |
| | | AGACACAAAAGCAAG |
| SEQ ID | GI 51479142 NM_001003703 Homo sapiens | CTCAAAGAGCGCATG |
| NO: 2774 | ATP synthase, H+ transporting, | CGTACAGAGCAAGGT |
| | mitochondrial Fo complex subunit F6 | TTCAAGGAGGGGAAA |
| | (ATP5J), transcript [symbol] | AAGAACGGGTCAAAT |
| SEQ ID | GI 31343534 NM_178818 Homo sapiens | CCTGCACATGAGGAT |
| NO: 2775 | CKLF like MARVEL transmembrane domain containing 4 | CCCCCAGATCAACTG |
| | (CMTM4), transcript variant 1, mRNA [symbol] | GAATCTGACAGATTT |
| | | GGTCAACACTGGACT |
| SEQ ID | GI 695917236 NM_021010 Homo sapiens | GAAATGGACTCTCTG |
| NO: 2776 | defensin alpha 5 | CTCTTAGAACCTCAG |
| | (DEFA5), mRNA | GTTCTCAGGCAAGAG |
| | | CCACCTGCTATTGCC |
| SEQ ID | GI 590122055 NM_021649 Homo sapiens | AGGGATGGGGTGGAG |
| NO: 2777 | toll like receptor adaptor molecule 2 | AATGGGTCATGTCAT |
| | (TICAM2), mRNA | GTAATGAGAAAAGCC |
| | | CTCTTCGGGATCATG |
| SEQ ID | GI 315365062 HQ384162 Homo sapiens | CCTAAGCTGAGAGTT |
| NO: 2778 | cell death-inducing DFFA-like effector a | CGGTTTTCAGAGATC |
| | (CIDEA) gene, complete cds | TGGGGGAGTAGCATC |
| | | AATGGAGGGGTTTGC |
| SEQ ID | GI 10799005 AF261108 Homo sapiens | GGCATCTCCTACTAC |
| NO: 2779 | type XV collagen | CTGAGCTGTTTTAGG |
| | (COL15A1) gene, promoter sequence and exon 1, | CAGGGAACAAAGCAC |
| | | AGATAAGCCCTCCTG |
| SEQ ID | GI 117606543 EF064746 Homo sapiens | TCAGGTAGCTCCGAG |
| NO: 2780 | complement component | TGGGGAGATGACCAA |
| | (3d/Epstein Barr virus) receptor 2 | GTCATGCCACCTAAA |
| | (CR2) gene, complete cds | ACATGTCACCAAAAA |
| SEQ ID | GI 57864635 AY882422 Homo sapiens | CAGGACACTTGCTGC |
| NO: 2781 | flavin containing monooxygenase 4 | CACCTCTTACCTGGT |
| | (FMO4) gene, complete cds | CTCCCTCCTTCCACT |
| | | CTGTTCTACAATCTA |
| SEQ ID | GI 342187237 NG_029217 Homo sapiens | CTCACTGTGCTGAAG |
| NO: 2782 | major histocompatibility complex, class 1, A | CCCAAAGGGAAACAT |
| | (HLA-A), RefSeqGene on chromosome 6 | CCCTCATCCAAGCCA |
| | | AACCAGAAGCAATAT |
| SEQ ID | GI 1147623323 NG_029437 Homo sapiens | TCCTTTGCAGTCTCT |
| NO: 2783 | mitogen-activated protein kinase kinase 6 | CCATGTCTGCATCAC |
| | (MAP2K6), RefSeqGene on chromosome 17 | TGGGGACTGATTCTG |
| | | AGGTTTCAGTGGTTG |
| SEQ ID | GI 270483830 NG_016181 Homo sapiens | CTACACGGGAAAGCT |
| NO: 2784 | sphingosine-1-phosphate receptor 1 | GAATATCAGCGCGGA |
| | (SIPR1), RefSeqGene on chromosome 1 | CAAGGAGAACAGCAT |
| | | TAAACTGACCTCGGT |
| SEQ ID | GI 40794510 AY497547 Homo sapiens | AGATGAAGAAGACAG |
| NO: 2785 | chemokine-like receptor 1 | TGAACATGGTCTGGT |
| | (CMKLR1) mRNA, complete cds | TCCTCAACCTGGCAG |
| | | TGGCAGATTTCCTGT |
| SEQ ID | GI 10798850 AF084559 Homo sapiens | TTCCTCAACTTCATC |
| NO: 2786 | fatty acid desaturase 2 | AGGTTCCTGGAGAGC |
| | (FADS2) mRNA, complete cds | CACTGGTTTGTGTGG |
| | | GTCACACAGATGAAT |
| SEQ ID | GI 209413750 NM_006038 Homo sapiens | CTCAACCAATGGCTG |
| NO: 2787 | spermatogenesis associated 2 | CTTTGTTGTCTGACA |
| | (SPATA2), transcript variant 1, mRNA | AGGGAGAAGGTGGCA |
| | | CTTCCGTTCAGATTC |
| SEQ ID | GI 46091142 AB110934 Homo sapiens | TTCTATGCTCCATAC |
| NO: 2788 | MRPS18B gene for MRPS18B protein, | ACAGGTTAGCCCATC |
| | complete cds, cell_line: LKT3 | ATCCCTGCACCACCA |
| | | GAGAGCTTTTCCTTG |
| SEQ ID | GI 5821735 AF155233 Homo sapiens | CAGGAATGCATATGG |
| NO: 2789 | snail zinc finger protein | GAAGCACCTGGCAAA |
| | (SNAIl) gene, complete cds | GTGCCGGTACCTGCT |
| | | AAACTCTCACAAAAA |
| SEQ ID | GI 212549702 NG_008951 Homo sapiens | CACTGTCAACTGCAG |
| NO: 2790 | ATR serine/threonine kinase | CCCAGCAGGAAGGAA |
| | (ATR), RefSeqGene on chromosome 3 | ACGAGCAGGTATAAA |
| | | GACCTTAACCTCACT |
| SEQ ID | GI 237858602 NG_012146 Homo sapiens | TGCCTAAGTCCAGCC |
| NO: 2791 | period circadian clock 2 | AAGGTTTAAGGAGAA |
| | (PER2), RefSeqGene on chromosome 2 | AGGAATTAGGCCCCA |
| | | CCTTTTGAGGAAGGA |
| SEQ ID | GI 237858805 NG_012139 Homo sapiens | CCAACTTCTTGCCCT |
| NO: 2792 | uncoupling protein 1 | ACCTCTCTCTTTCTT |
| | (UCP1), RefSeqGene on chromosome 4 | CCCCATCACCCAATT |
| | | TCCCAGTCAGTCAGG |
| SEQ ID | GI 75709215 NM_181866 Homo sapiens | TGCAGGTCAACGTGA |
| NO: 2793 | acyl-CoA thioesterase 7 | TGTCCGAAAACATCC |
| | (ACOT7), transcript variant hBACHd, mRNA | TCACAGGTGCCAAAA |
| | | AGCTGACCAATAAGG |
| SEQ ID | GI 927028873 NM_004836 Homo sapiens | TCTACAGCGTACCCA |
| NO: 2794 | eukaryotic translation initiation | AAAAACTGTTAGAGC |
| | factor 2 alpha kinase 3 | TGTCGGACCTCGCAG |
| | (EIF2AK3), transcript variant 1, mRN [symbol] | TGGCAATGAGAAGTG |
| SEQ ID | GI 261337164 NM_206943 Homo sapiens | GTACTTTCCAGCCCA |
| NO: 2795 | latent transforming growth | GAAGGGGATTTCAGG |
| | factor beta binding protein 1 | AGAGCAGTCCACTGA |
| | (LTBP1), transcript variant 1, mRN [symbol] | AGGTTCTTTCCCTTT |
| SEQ ID | GI 523498705 NM_024923 Homo sapiens | GAAAACATCCTTCTG |
| NO: 2796 | nucleoporin 210 | AACCCGGCCTATGAC |
| | (NUP210), mRNA | GTCTACCTGATGGTG |
| | | GGAACCTCCATTCAC |
| SEQ ID | GI 209862906 NM_006475 Homo sapiens | AGAGACGGTCACTTC |
| NO: 2797 | periostin | ACACTCTTTGCTCCC |
| | (POSTN), transcript variant 1, mRNA | ACCAATGAGGCTTTT |
| | | GAGAAACTTCCACGA |
| SEQ ID | GI 324072795 NM_005402 Homo sapiens | TTGTGTTTCTTGGGC |
| NO: 2798 | RAS like proto-oncogene A | AGTCTTTCTTGAAAT |
| | (RALA), mRNA | TGAAGAGGTGAAATG |
| | | GGGGTGGGGAGTGGG |
| SEQ ID | GI 984290537 NM_001319196 Homo sapiens | TAAAAAGGAGCTGCC |
| NO: 2799 | S100 calcium binding protein A8 | TCTCAGCCCTGCATG |
| | (S100A8), transcript variant 1, mRNA | TCTCTTGTCAGCTGT |
| | | CTTTCAGAAGACCTG |
| SEQ ID | GI 1220516002 NM_023003 Homo sapiens | TGTTGGTTGTGTGTC |
| NO: 2800 | transmembrane 6 superfamily member 1 | TCCTCCTGGCAACTG |
| | (TM6SF1), transcript variant 1, mRNA | GATTTTGCCTGTTCA |
| | | GAGGTTTGATTGCTT |
| SEQ ID | GI 117606573 EF064761 Homo sapiens | AAAGAGTGAATATGT |
| NO: 2801 | chemokine | GTCGCATCCAGAGAG |
| | (C-C motif) ligand 28 | CTGATGGGGATTGTG |
| | (CCL28) gene, complete cds | ACTTGGCTGCTGTCA |
| SEQ ID | GI 189217848 NG_007931 Homo sapiens | GGAGCAAGGTGGTGG |
| NO: 2802 | cytochrome P450 family 2 | GATAAGACTGAATCC |
| | subfamily J member 2 | ACTGTATTTGCTCCG |
| | (CYP2J2), RefSeqGene on chromosome 1 | ATGGGGACCATTAGG |
| SEQ ID | GI 1028224151 NG_008493 Homo sapiens | GGAAGGTCTCCATGG |
| NO: 2803 | estrogen receptor 1 | AGAAGGTGTGCTTTG |
| | (ESR1), RefSeqGene on chromosome 6 | AGATGAACACTGAGG |
| | | AAGTGGTGGGATCAC |
| SEQ ID | GI 1003701615 NG_008076 Homo sapiens | CGGACACTCCTCTTG |
| NO: 2804 | growth differentiation factor 5 | CTGATGCCACCATCT |
| | (GDF5), RefSeqGene on chromosome 20 | GGTTGAATTGTGTCA |
| | | ATCTCCCTAGTTGGA |
| SEQ ID | GI 160358379 NG_007100 Homo sapiens | TATCCTCAAATGCAA |
| NO: 2805 | methylmalonyl-CoA mutase | ACTGCGGAGTCTGCC |
| | (MUT), RefSeqGene on chromosome 6 | ACAAACGCGTTGGCT |
| | | TTAAAACTCATCCCT |
| SEQ ID | GI 237858758 NG_012120 Homo sapiens | GAACAGAGCATGCAA |
| NO: 2806 | RAD51 recombinase | TCCATCCCCATGATC |
| | (RAD51), RefSeqGene on chromosome 15 | TTACCCTGGGAGCTT |
| | | GTCCACTATCCTGTT |
| SEQ ID | GI 257153311 NG_012800 Homo sapiens | AAGGGAGCCCAGAAA |
| NO: 2807 | regulator of G protein signaling 2 | TGGAGGACATTTCAT |
| | (RGS2), RefSeqGene on chromosome 1 | TCTTTTTCCTGAGGG |
| | | GAAGGACTGTGACCT |
| SEQ ID | GI 1018443413 NG_027814 Homo sapiens | GTGCGAGCTGTTACG |
| NO: 2808 | SATB homeobox 1 | CTGGAAAGAAGATCC |
| | (SATB1), RefSeqGene on chromosome 3 | TTCTCCAGAAAACAG |
| | | AACCCTGTGGGAGAA |
| SEQ ID | GI 340523103 NG_029468 Homo sapiens | GCAGGGGCTGTTGTA |
| NO: 2809 | SWI/SNF related, matrix associated, | GAATCCCTCTCAACT |
| | actin dependent regulator of | TGTCTGTGTCTTAAA |
| | chromatin, subfamily d, m [symbol] | CCCGGAGTACCAACC |
| SEQ ID | GI 81230486 NM_001037162 Homo sapiens | GGACATGGTTTTGCT |
| NO: 2810 | acyl-CoA thioesterase 6 | GTGCTTGCCCTGGCT |
| | (ACOT6), mRNA | TATTTCAGATTTGAA |
| | | GACCTCCCCGAAGAT |
| SEQ ID | GI 38505219 NM_017888 Homo sapiens | CCACTCAGCCCTGCT |
| NO: 2811 | acyl-CoA synthetase medium | CTGCTCAGCTGAAGC |
| | chain family member 5 | AGAAAACAGAGACCT |
| | (ACSM5), transcript variant 1, mRNA | TTTGCATTACTTTGG |
| SEQ ID | GI 114731556 DQ866809 Homo sapiens | TGTAATCTCAAAGAA |
| NO: 2812 | endoglin | CAGGAGACCTGAGGG |
| | (Osler-Rendu-Weber syndrome 1) | AAGAGAGGCGGACAA |
| | (ENG) gene, complete cds | AGGACACACACTCCT |
| SEQ ID | GI 300116187 NG_023252 Homo sapiens | GTCCTTCTCAAAACA |
| NO: 2813 | interleukin 4 | CCTAAACTTGGGAGA |
| | (IL4), RefSeqGene on chromosome 5 | ACATTGTCCCCCAGT |
| | | GCTGGGGTAGGAGAG |
| SEQ ID | GI 340805855 NG_029505 Homo sapiens | TAGGGGATCAGAATG |
| NO: 2814 | methyl-CpG binding domain protein 1 | GTGACAGTTAGGGAT |
| | (MBD1), RefSeqGene on chromosome 18 | GGCATGATCCCGGGA |
| | | CAAGAACAGAGAGAA |
| SEQ ID | GI 353409895 NG_030365 Homo sapiens | TCCAGGGAACTGTGG |
| NO: 2815 | POU class 2 homeobox 2 | TCTTACATGGGGGAT |
| | (POU2F2), RefSeqGene on chromosome 19 | ACAAGCACCCCTAGA |
| | | TACACACCGACATAG |
| SEQ ID | GI 343488503 NG_029728 Homo sapiens | CCACATCTCTTCCAC |
| NO: 2816 | protein kinase cAMP-activated | CTTCTAGAGGCCACC |
| | catalytic subunit beta | TGCTTTCCTTGACTC |
| | (PRKACB), RefSeqGene on chromosom [symbol] | ATGATGCCATTCCTC |
| SEQ ID | GI 393662586 NM_002754 Homo sapiens | TCAAGCAGAGGACAG |
| NO: 2817 | mitogen-activated protein kinase 13 | AAGGGTCCTTCTCCT |
| | (MAPK13), transcript variant 1, mRNA | TATGTGGGAAATGGG |
| | | CCTAGTAGATGCAGA |
| SEQ ID | GI 53759150 NM_005063 Homo sapiens | GGGGCCTGAGTGGAG |
| NO: 2818 | stearoyl-CoA desaturase | GATTATCAGTATCAC |
| | (SCD), mRNA | GATTTGCAGGATTCC |
| | | CTTCTGGGCTTCATT |
| SEQ ID | GI 543173138 NM_018202 Homo sapiens | AACTGGTGGGTAGCT |
| NO: 2819 | transmembrane protein 57 | GGGCTGTCCGTTACC |
| | (TMEM57), transcript variant 1, mRNA | ACCACAGCTACATCT |
| | | TCTGTTTCTTTGAAT |
| SEQ ID | GI 226509596 NG_011798 Homo sapiens | TTGGACAGGCATAGC |
| NO: 2820 | ATP binding cassette subfamily C member 2 | TGCTTGGAGATGTCT |
| | (ABCC2), RefSeqGene on chromosome 10 | CTCAGCAGCAGCTGG |
| | | GTCTTTCTTTTTACT |
| SEQ ID | GI 341865533 NG_029560 Homo sapiens | CACAGCGGAATTTCT |
| NO: 2821 | aquaporin 4 | GGCCATGCTTATTTT |
| | (AQP4), RefSeqGene on chromosome 18 | TGTTCTCCTCAGCCT |
| | | GGGATCCACCATCAA |
| SEQ ID | GI 197209823 NG_008387 Homo sapiens | AAACGTATGTTGCAA |
| NO: 2822 | interleukin 1 receptor | TCTCGGGCTCTACTT |
| | associated kinase 1 | GTGGACTTTGTTGCA |
| | (IRAK1), RefSeqGene on chromosome X | CCGAAAGCCTTGAGC |
| SEQ ID | GI 194353939 NG_008255 Homo sapiens | ATGCCTTCTCGCTAT |
| NO: 2823 | solute carrier family 19 member 2 | GCTGCTATCTATAAT |
| | (SLC19A2), RefSeqGene on chromosome 1 | GGTGGCGTGGAGGCC |
| | | GTTTCAACCTTACTG |
| SEQ ID | GI 336455046 NG_029166 Homo sapiens | AAGAAGGATTAGCAG |
| NO: 2824 | SRY-box 4 | CCAGGTCTACTATGA |
| | (SOX4), RefSeqGene on chromosome 6 | ATCCGGAGGCCCCAT |
| | | TCTCTTCTGCTACCA |
| SEQ ID | GI 156523973 NM_001296 Homo sapiens | GACCAAATTTTGGTC |
| NO: 2825 | atypical chemokine receptor 2 | TGGTGGGAACAGATG |
| | (ACKR2), mRNA | GGAACCAGCTCAATT |
| | | GGGTGTCCACTCAAA |
| SEQ ID | GI 1084352529 NM_000755 Homo sapiens | GGGTCAGCTCCGATT |
| NO: 2826 | carnitine O-acetyltransferase | TGCTGCCAAACTCAT |
| | (CRAT), transcript variant 1, mRNA | TGAGGGTGTGTTGGA |
| | | TTTCAAGGTCATGAT |
| SEQ ID | GI 394582046 NM_003877 Homo sapiens | AAAAGTAGGTCACAT |
| NO: 2827 | suppressor of cytokine signaling 2 | TTCAAGTAGGTCATA |
| | (SOCS2), transcript variant 1, mRNA | TTTCGGGGGCGGGTG |
| | | CGCAGACAAGGAGAT |
| SEQ ID | GI 523425747 NM_004599 Homo sapiens | AGCTATAGAGTCCTT |
| NO: 2828 | sterol regulatory element | GGTGAAACCTCAGGC |
| | binding transcription factor 2 | CAAGAAGAAGGCTGG |
| | (SREBF2), transcript variant 1, mRN [symbol] | AGACCAGGAAGAAGA |
| SEQ ID | GI 15021772 AF400075 Homo sapiens | ACTGCCTCATAGAAA |
| NO: 2829 | coagulation factor II | CGAACTCAGCCGAGA |
| | (thrombin) receptor-like 1 | TCACAACCTTCTCAG |
| | (F2RL1) gene, complete cds | GGTACAGGTGAAGGA |
| SEQ ID | GI 182739 L10820 Human | CACCTTTCGGATCAG |
| NO: 2830 | N-formyl peptide receptor | TGATGAGTCTCCACG |
| | (FPR1) gene, complete cds and Alu repeats | TGAAGACACTGGTTC |
| | | TGCATGGTGCATAGG |
| SEQ ID | GI 300069049 NG_023225 Homo sapiens | GTGGGCCTACTGTCT |
| NO: 2831 | acetyl-CoA acyltransferase 1 | TCCCTGATATGGTCA |
| | (ACAA1), RefSeqGene on chromosome 3 | TTCTGTCTCCTTGAA |
| | | GTGCTAGCCCTACCT |
| SEQ ID | GI 974987497 NG_011800 Homo sapiens | GCTCTAAGAACAGCA |
| NO: 2832 | ceruloplasmin | AAAGGATGGATGGAG |
| | (CP), RefSeqGene on chromosome 3 | CAGGGTGAGCAGAAG |
| | | GTGTGGAAAGCTAGA |
| SEQ ID | GI 339409221 NG_029376 Homo sapiens | ACATTCTTACAAGCA |
| NO: 2833 | interleukin 17 receptor C | ATGCCTCCAAAAGCA |
| | (IL17RC), RefSeqGene on chromosome 3 | AAGGAGGATGGGGCA |
| | | ATCTCCTTGATCCCC |
| SEQ ID | GI 19072753 AF475085 Homo sapiens | TACTTCTTGGGACTT |
| NO: 2834 | mucolipin-3 | CTACCATGCTCGTGT |
| | (MCOLN3) mRNA, complete cds | GGCTTGGAGTCATCC |
| | | GATACCTCGGTTTCT |
| SEQ ID | GI 327412387 NG_028268 Homo sapiens | TGTTGATGCCAAGGC |
| NO: 2835 | ATP binding cassette subfamily C member 1 | TGAAGAAGACTGGAT |
| | (ABCC1), RefSeqGene on chromosome 16 | TTGCAGGCAAAAGAC |
| | | TGTCATTTGAGGCCT |
| SEQ ID | GI 532691848 NG_009095 Homo sapiens | TCATTGGCTCCATCA |
| NO: 2836 | calcium voltage-gated | CCTCCATTGGGTACT |
| | channel subunit alphal F | ACATGGTTCTGTCAC |
| | (CACNA1F), RefSeqGene on chromosome X | CTCAACTGACAACGC |
| SEQ ID | GI 190358505 NG_008040 Homo sapiens | AGTAACAGACAGATG |
| NO: 2837 | colony stimulating factor 2 | GATGTCCCGAGGCTT |
| | receptor beta common subunit | GGGGAAGTAACACGC |
| | (CSF2RB), RefSeqGene on chrome [symbol] | AGATGATTTGCACAA |
| SEQ ID | GI 291045457 NG_016717 Homo sapiens | TACATCATCCAGAGC |
| NO: 2838 | phosphoribosyl pyrophosphate synthetase 2 | GGCTGCGGGGAAATT |
| | (PRPS2), RefSeqGene on chromosome X | AACGACAACCTGATG |
| | | GAACTCCTCATCATG |
| SEQ ID | GI 226246635 NG_011759 Homo sapiens | ACACATTCAGACCCC |
| NO: 2839 | transporter 1, ATP binding | AGATCCTCCTCCACA |
| | cassette subfamily B member | AATTCAGAGACCCAA |
| | (TAP1), RefSeqGene on chromoso [symbol] | GCACCCACCAAATAG |
| SEQ ID | GI 756140821 NM_014358 Homo sapiens | TGGACTGTTGCTGGG |
| NO: 2840 | C-type lectin domain family 4 member E | ATCCCCATCCTATTT |
| | (CLEC4E), mRNA | CTCAGTGCCTGTTTC |
| | | ATCACCAGATGTGTT |
| SEQ ID | GI 815890844 NM_001836 Homo sapiens | TAAGACGGAACTTTG |
| NO: 2841 | chymase 1 | TGCTGACGGCTGCTC |
| | (CMAl), transcript variant 1, mRNA | ATTGTGCAGGAAGGT |
| | | CTATAACAGTCACCC |
| SEQ ID | GI 209915567 NM_018438 Homo sapiens | CCTGCGCAACCCGTG |
| NO: 2842 | F-box protein 6 | TGCTGAAGAGGATAT |
| | (FBX06), mRNA | GTTTGCATGGCAAAT |
| | | TGATTTCAATGGTGG |
| SEQ ID | GI 260166681 NM_138387 Homo sapiens | CACCGAGTGGCTCAA |
| NO: 2843 | glucose-6-phosphatase | CCTCATCTTCAAGTG |
| | catalytic subunit 3 | GTTTCTTTTTGGAGA |
| | (G6PC3), transcript variant 1, mRNA | CAGGCCCTTTTGGTG |
| SEQ ID | GI 225579056 NM_003049 Homo sapiens | GTGTTCATGTTGTTC |
| NO: 2844 | solute carrier family 10 member 1 | TTCATCATGCTCTCG |
| | (SLC10A1), mRNA | CTGGGCTGCACCATG |
| | | GAGTTCAGCAAGATC |
| SEQ ID | GI 115432768 DQ995342 Homo sapiens | GTCTGAACGATTTTA |
| NO: 2845 | ADAM metallopeptidase domain 33 | AACCATTGGCTACAC |
| | (ADAM33) gene, complete cds | CCCTGCCCCTCCTAG |
| | | AGAGCCCTCTTTGTT |
| SEQ ID | GI 66841738 DQ075211 Homo sapiens | AGGGGACCCACCTGA |
| NO: 2846 | c-src tyrosine kinase | GGTCCCACAGATATA |
| | (CSK) gene, complete cds | ATTTGGTGCCAGGAC |
| | | TAGAATCTGAGTCTG |
| SEQ ID | GI 18653447 AF478696 Homo sapiens | AACTAGTGGGTTCAG |
| NO: 2847 | coagulation factor II | GAGCTCGAGCTAGAA |
| | (thrombin) | GTGGTGAGAAGGGTT |
| | (F2) gene, complete cds | TGGATTTGGGGTCTA |
| SEQ ID | GI 54781374 AY795972 Homo sapiens | AGCACAGTTGTATAT |
| NO: 2848 | matrix metalloproteinase 7 | AGAGTGGCCACTAAT |
| | (matrilysin, uterine) | CCAGCCACACAGCAG |
| | (MMP7) gene, complete cds | CATTTCCATCTGCCT |
| SEQ ID | GI 571026712 NG_033872 Homo sapiens | TCTTCTAGGCAGCCA |
| NO: 2849 | cytochrome cl | TCAGACACGTTCAAT |
| | (CYC1), RefSeqGene on chromosome 8 | AACGGAGGTTCTGTG |
| | | CGAATGAGGGCATTT |
| SEQ ID | GI 299829203 NG_023197 Homo sapiens | TTTCTGACAGCATGA |
| NO: 2850 | parathyroid hormone like hormone | TTGGAAACGCAGGCC |
| | (PTHLH), RefSeqGene on chromosome 12 | CTTTTCCTTTCCGCA |
| | | CATATGGGCTAAGTC |
| SEQ ID | GI 237858758 NG_012120 Homo sapiens | TCCCTGTCTTCCTGA |
| NO: 2851 | RAD51 recombinase | AGCTGAAGCTATGTT |
| | (RAD51), RefSeqGene on chromosome 15 | CGCCATTAATGCAGA |
| | | TGGAGTGGGAGATGC |
| SEQ ID | GI 1024846748 NG_046980 Homo sapiens | TCCCCCGCATCCTTT |
| NO: 2852 | synaptonemal complex protein 2 | AATCCCTTCAGTATG |
| | (SYCP2), RefSeqGene on chromosome 20 | CCACCTTGCAAAATC |
| | | TCCAAGCCTAGGTAA |
| SEQ ID | GI 692314725 NM_002416 Homo sapiens | CGGTGAAGTACTAAG |
| NO: 2853 | C-X-C motif chemokine ligand 9 | CGCTAGAGGAAGCAG |
| | (CXCL9), mRNA | CCAAGTCGGTTAGTG |
| | | GAAGCATGATTGGTG |
| SEQ ID | GI 31317227 NM_004430 Homo sapiens | TGCTCTCTGCCACAG |
| NO: 2854 | early growth response 3 | CTTGCAGCATGGTCT |
| | (EGR3), transcript variant 1, mRNA | TGACTGAATGTACTG |
| | | TTCCTGTTAGCGTTA |
| SEQ ID | GI 238018064 NG_012194 Homo sapiens | GCCCTTCTCTTTCCC |
| NO: 2855 | cytochrome b5 reductase 3 | AAGACAGTAATCCTA |
| | (CYB5R3), RefSeqGene on chromosome 22 | GAGAGTGCAGGGCAG |
| | | GAAGTAGTCCCAAGA |
| SEQ ID | GI 1172222469 NG_053051 Homo sapiens | AGGCGGATTGTTGTT |
| NO: 2856 | heat shock protein family H | AGCGACCACGGAAGC |
| | (HspllO) member 1 | AGTCCTATGAAAGAT |
| | (HSPH1), RefSeqGene on chromosome 13 | TAGCCCACCTCTTTT |
| SEQ ID | GI 224809341 NG_011508 Homo sapiens | TTGATCAGGTCAGCA |
| NO: 2857 | insulin like growth factor | ATGTGCACCTATGTG |
| | binding protein 3 | ACTGGTCCTTGCCTT |
| | (IGFBP3), RefSeqGene on chromosome 7 | TTTTGGGTTGTGTCC |
| SEQ ID | GI 209447063 NG_008722 Homo sapiens | ACATTTGAATTGAGC |
| NO: 2858 | lysyl oxidase | CTTGAAGTCTGGGGA |
| | (LOX), RefSeqGene on chromosome 5 | ATGCACCGGGGAGCA |
| | | TTAAGGAAAGGAAGC |
| SEQ ID | GI 601984942 NG_034078 Homo sapiens | TGGGGTCCTGACTAG |
| NO: 2859 | myelin associated glycoprotein | GTGGTTTTCACCCAA |
| | (MAG), RefSeqGene on chromosome 19 | AAAGGTATTAGGGGT |
| | | TACATTGTGTGCCCT |
| SEQ ID | GI 1147399622 NG_012448 Homo sapiens | AGCATGTTTAAACAG |
| NO: 2860 | nodal growth differentiation factor | GGACCACCTACACAC |
| | (NODAL), RefSeqGene on chromosome 10 | TAGGCCACATGGTGA |
| | | TTTGGTAGACCTGGG |
| SEQ ID | GI 237858771 NG_012125 Homo sapiens | GACCTTCAGGACAAT |
| NO: 2861 | selectin P | GGACAGCAGTAACTC |
| | (SELP), RefSeqGene on chromosome 1 | CAGCATGCAGAGGTA |
| | | AGGTGAAAAGGAGCA |
| SEQ ID | GI 257467583 NG_012852 Homo sapiens | TCACTTCTATTTCAC |
| NO: 2862 | signal transducer and | TACGCCACTGCTCAA |
| | activator of transcription 4 | AAACCTGCAGTTACC |
| | (STAT4), RefSeqGene on chromosome 2 | CCCAGTCACGCTTCA |
| SEQ ID | GI 38708325 NM_001896 Homo sapiens | CGAATGTAAAAGGCT |
| NO: 2863 | casein kinase 2 alpha 2 | CACTTGCCTTTGGCT |
| | (CSNK2A2), mRNA | TCCTGTTGACTTCTT |
| | | CCCGACCCAGAAAGC |
| SEQ ID | GI 735661405 NM_002201 Homo sapiens | TCCGCCCCTGACTTC |
| NO: 2864 | interferon stimulated exonuclease gene 20 | ACTTGATAACAAACT |
| | (ISG20), transcript variant 1, mRNA | AGAAACTGAAACAGG |
| | | GTCGGGATGCCGATG |
| SEQ ID | GI 164695749 AK315559 Homo sapiens | ACGAAAGAAGAAGAA |
| NO: 2865 | cDNA, FLI96630, Homo sapiens | GAGCAAAGGGCCTTC |
| | methionyl aminopeptidase 2 | TGCAGCAGGGGAACA |
| | (METAP2), mRNA | GGAACCTGATAAAGA |
| SEQ ID | GI 219842351 NM_002522 Homo sapiens | GCCACCTGTTCTCAA |
| NO: 2866 | neuronal pentraxin 1 | AAGCATGTTGGGGAC |
| | (NPTX1), mRNA | CAGGAGGTAAAACTG |
| | | GCCATTTATGGTGAA |
| SEQ ID | GI 115387093 NM_003000 Homo sapiens | CCCCGTATCAAGAAA |
| NO: 2867 | succinate dehydrogenase | TTTGCCATCTATCGA |
| | complex iron sulfur subunit B | TGGGACCCAGACAAG |
| | (SDHB), mRNA | GCTGGAGACAAACCT |
| SEQ ID | GI 685156953 NG_034254 Homo sapiens | CAAGTCATTGTTGAC |
| NO: 2868 | cyclin D2 | ATTGCTAGGGGACGA |
| | (CCND2), RefSeqGene on chromosome 12 | GGGGTGCCTATGCAG |
| | | CCAGCTTATAAGCTA |
| SEQ ID | GI 334191656 NG_029012 Homo sapiens | AGCATAAGAATCACC |
| NO: 2869 | integrin subunit beta 1 | TGGAGTCCAGGCCCC |
| | (ITGB1), RefSeqGene on chromosome 10 | ATGCTCAGGGATTGT |
| | | GGATTTTAAGCCTAG |
| SEQ ID | GI 169808406 NG_007450 Homo sapiens | TGCTGGCTAAGCTTG |
| NO: 2870 | leptin | TCATAAGTTCAGTGT |
| | (LEP), RefSeqGene on chromosome 7 | GACACCCTTTCCCGT |
| | | GATGGGCTGATTACA |
| SEQ ID | GI 1024846809 NG_046989 Homo sapiens | CGGCAGGATCCTGGC |
| NO: 2871 | matrix metallopeptidase 14 | TTCAGCTGGATCACA |
| | (MMP14), RefSeqGene on chromosome 14 | CCTCTTACCACATTC |
| | | CAAGTTCCTAGTTTC |
| SEQ ID | GI 224809428 NG_011537 Homo sapiens | CCTCATTTGAACGCG |
| NO: 2872 | myosin heavy chain 3 | ATCAGCATGGGTGCC |
| | (MYH3), RefSeqGene on chromosome 17 | TTTCTGGTCCACTCA |
| | | ACACCTTTGATCTTG |
| SEQ ID | GI 345842421 NG_029949 Homo sapiens | TGAAAGGGAAAGAAC |
| NO: 2873 | TNF superfamily member 13 | CAAAAAGCACCTGAG |
| | (TNFSF13), RefSeqGene on chromosome 17 | AGTGCCAAGAAGTCC |
| | | TGACCGACACACTCT |
| SEQ ID | GI 4572463 AF123653 Homo sapiens | ACCTGACCGTGCTGG |
| NO: 2874 | FEZI | AGAACCCCATAACCT |
| | (FEZl) gene, complete cds | CTGCATCTATTAGTG |
| | | GGAAAGCAGCTTTTC |
| SEQ ID | GI 953768433 NG_016863 Homo sapiens | ACGATTGCTTTCGTT |
| NO: 2875 | delta like non-canonical Notch ligand 1 | CGTTAATTCTCACAC |
| | (DLK1), RefSeqGene on chromosome 14 | ACCACATCCGACTCG |
| | | CACTCACATCCAGCT |
| SEQ ID | GI 170014708 NG_007458 Homo sapiens | TGGTAAACGTCAGTC |
| NO: 2876 | notch 1 | TCGTCCCTGGTTTCT |
| | (NOTCH1), RefSeqGene on chromosome 9 | GACGTAATTACCTCC |
| | | CGTGTTACCAGGACG |
| SEQ ID | GI 194018405 NG_008205 Homo sapiens | TTCCTGGTCTACCAC |
| NO: 2877 | phosphoenolpyruvate carboxykinase 1 | TGGACCACATGACTC |
| | (PCK1), RefSeqGene on chromosome 20 | CAAAACCAACACTGT |
| | | GACTGACGTCCTCTA |
| SEQ ID | GI 671744853 NG_034216 Homo sapiens | TCTGTATCTTGTTTC |
| NO: 2878 | vascular endothelial growth factor C | TATTCTGGCCCTGGC |
| | (VEGFC), RefSeqGene on chromosome 4 | TTGTGTTCTGCCTTT |
| | | CCAAAGCCCTCTCCA |
| SEQ ID | GI 116069823 EF050529 Homo sapiens | AGCTGGAGAAGTCCA |
| NO: 2879 | caspase 10, apoptosis- | GCTCAGCCTCAGATG |
| | related cysteine peptidase | TTTTTGAACATCTCT |
| | (CASP10) gene, complete cds | TGGCAGAGGATCTGC |
| SEQ ID | GI 117606585 EF064767 Homo sapiens | CCAGAGTCCTGGGGA |
| NO: 2880 | chemokine | TGGCCCAGTCTGAGT |
| | (C-C motif) ligand 17 | TCTTCATGGAAAATT |
| | (CCL17) gene, complete cds | GTCCTCTGAATCCAC |
| SEQ ID | GI 2246432 U45984 Homo sapiens | CTGGAAGTGCTTCGG |
| NO: 2881 | CCR6 chemokine receptor | AAAGAAGGGGACAAT |
| | (CMKBR6) gene, complete cds | GGCAGAACAGGTGTT |
| | | GGTGACAATTGTCAC |
| SEQ ID | GI 117606537 EF064743 Homo sapiens | AAGCAGCAGAGTGCT |
| NO: 2882 | chemokine | TGATGTCATTGGAGC |
| | (C-X-C motif) ligand 13 | TTGGAGAAGGGGAGA |
| | (B-cell chemoattractant) | CAGGAAGACAAGAAG |
| | (CXCL13) gene, complete cds | |
| SEQ ID | GI 12584865 AF307860 Homo sapiens | TCTTGAGGAGCATTA |
| NO: 2883 | CYR61 protein | AGGTATTTCGAAACT |
| | (CYR61) gene, complete cds | GCCAAGGGTGCTGGT |
| | | GCGGATGGACACTAA |
| SEQ ID | GI 237858769 NG_012123 Homo sapiens | AGTAGAGACATGCTT |
| NO: 2884 | C-C motif chemokine ligand 2 | CCAACACTCAGAAGC |
| | (CCL2), RefSeqGene on chromosome 17 | CTATGTGAACACTCA |
| | | GCCAGCAAAGCTGGG |
| SEQ ID | GI 324021724 NG_028154 Homo sapiens | TGCTTGGGTCCTTCA |
| NO: 2885 | fatty acid binding protein 5 | GAGAAGTGCACAAAA |
| | (FABP5), RefSeqGene on chromosome 8 | TCAGCTGCATGCTGG |
| | | TTTTACAGCTCTGTG |
| SEQ ID | GI 224809344 NG_011510 Homo sapiens | TCAGTTGTTGGTCTT |
| NO: 2886 | lanosterol synthase | TGAGCTCCTGTTAGT |
| | (LSS), RefSeqGene on chromosome 21 | CCAGCCGTCTCTCGT |
| | | GGTCTCTTTTCTCCT |
| SEQ ID | GI 225007577 NG_011607 Homo sapiens | GTTTGGTGCTCTCTG |
| NO: 2887 | MER proto-oncogene, tyrosine kinase | TGGGTTTAAGGATTT |
| | (MERTK), RefSeqGene on chromosome 2 | TTCTTCCTTCCAAGG |
| | | ATGGGCATGGAGGGC |
| SEQ ID | GI 225543321 NG_011651 Homo sapiens | CTACAACCCCCAGCT |
| NO: 2888 | NLRfamily pyrin domain containing 1 | TACAGATGAGAAAAC |
| | (NLRP1), RefSeqGene on chromosome 17 | TGAGGCTCATAGGGG |
| | | CTAATGGCCAGCATC |
| SEQ ID | GI 153792589 NM_001017963 Homo sapiens | ACCAAGAAAAGGCCC |
| NO: 2889 | heat shock protein 90 alpha family | AAGTGTTTCTCTGGC |
| | class A member 1 | ATCTGATGGTGTCTG |
| | (HSP90AA1), transcript variant 1, [symbol] | GATCCACCACTCTAC |
| SEQ ID | GI 295842487 NM_014470 Homo sapiens | AGCCAATGAGAAGGA |
| NO: 2890 | Rho family GTPase 1 | AACCTCATCTTTGCA |
| | (RND1), mRNA | TAGCCCATGCCTCAT |
| | | GGAGAGGTGACATCA |
| SEQ ID | GI 385198082 NM_005226 Homo sapiens | TACTCCAAGAAGTAC |
| NO: 2891 | sphingosine-1-phosphate receptor 3 | ATTGCCTTCTGCATC |
| | (S1PR3), mRNA | AGCATCTTCACGGCC |
| | | ATCCTGGTGACCATC |
| SEQ ID | GI 1168024008 KY497359 Homo sapiens | AGTTTCCCGTTCCTC |
| NO: 2892 | HLA-E | TCGTAACCTGGTCAT |
| | (HLA-E) gene, complete cds | GTGTCCTTCTTCCTG |
| | | GATACTCATGACGCA |
| SEQ ID | GI 27501939 AY208161 Homo sapiens | GTGGTCCAGAGGAAG |
| NO: 2893 | transforming growth factor, beta 3 | CTGGAGGTAAATGTA |
| | (TGFB3) gene, complete cds | GATTCCCTGGTCCCA |
| | | CCCACCCTTATAGAA |
| SEQ ID | GI 56157779 AY835629 Homo sapiens | TTTTGAGGACACAGG |
| NO: 2894 | superoxide dismutase 1, soluble | CCTAGAGCAGTTAAG |
| | (amyotrophic lateral sclerosis 1 | CAGCTTGCTGGAGGT |
| | (adult)) | TCACTGGCTAGAAAG |
| | (SOD1) gene, [symbol] complete [symbol] | |
| SEQ ID | GI 1024846791 NG_047016 Homo sapiens | TTTCTGCTTCAGTCA |
| NO: 2895 | bactericidal/permeability- | CATCTTTGGGTGCAA |
| | increasing protein | TTAGCACTTCCCCAT |
| | (BPI), RefSeqGene on chromosome 20 | GACTCTCCCAGCTCT |
| SEQ ID | GI 350606343 NG_030328 Homo sapiens | TGTCTGTGAGACAGG |
| NO: 2896 | neuropilin 1 | AGATGGGTCATATGG |
| | (NRP1), RefSeqGene on chromosome 10 | GCTGGGTCCCGATAT |
| | | GTGAATGGCTCATTA |
| SEQ ID | GI 42794751 NM_004457 Homo sapiens | TAAGGGGAGTGTTAG |
| NO: 2897 | acyl-CoA synthetase | GAGCAGCCAGGACTG |
| | long chain family member 3 | TGTAGTGTGTGTTTG |
| | (ACSL3), transcript variant 1, mRNA | GTTGCATCACAAACA |
| SEQ ID | GI 345199309 NM_002167 Homo sapiens | CAGGTCACTGTAGCG |
| NO: 2898 | inhibitor of DNA binding 3, HLH protein | GGACTTCTTTTGGTT |
| | (ID3), mRNA | TTCTTTCTCTTTGGG |
| | | GCACCTCTGGACTCA |
| SEQ ID | GI 55953136 NM_002163 Homo sapiens | ACCAGCCACTTGGAA |
| NO: 2899 | interferon regulatory factor 8 | GACGAGGTTACGCTG |
| | (IRF8), mRNA | TGCTTTGAATAAGAG |
| | | CCCAGATTTTGAGGA |
| SEQ ID | GI 356582493 NM_001244 Homo sapiens | GAGAGACAATGATGA |
| NO: 2900 | TNF superfamily member 8 | CCCTGGAAGTGGGGA |
| | (TNFSF8), transcript variant 1, mRNA | GGAGGACTTCTGAGA |
| | | AACAGCAACCTCTCT |
| SEQ ID | GI 1150273800 NG_052917 Homo sapiens | GAGATAAAGGGAGAG |
| NO: 2901 | adenosine A1 receptor | AGTGGTCAGAGATAA |
| | (ADORAl), RefSeqGene on chromosome 1 | TGGGTCACTGGCCCA |
| | | GGTACAGCATGGAGG |
| SEQ ID | GI 1048072306 NG_051267 Homo sapiens | TCTAGTCTGGGCCCC |
| NO: 2902 | arsA arsenite transporter, | CCAAGTAGAAATAGG |
| | ATP-binding, homolog 1 | GGTATCTTTGGGTTG |
| | (bacterial) | GGTAACCATCTCTGG |
| | (ASNAl), RefSeqGene on chr [symbol] | |
| SEQ ID | GI 292658840 NG_016861 Homo sapiens | GCATACTTGCCTCTT |
| NO: 2903 | C-X-C motif chemokine ligand 12 | CTGGACAGCTGATCC |
| | (CXCL12), RefSeqGene on chromosome 10 | TTAGCAGGGAAAGGT |
| | | GCTCCCTTTTCTCTG |
| SEQ ID | GI 224809263 NG_011469 Homo sapiens | GAGGCTCCTCATGCA |
| NO: 2904 | microsomal triglyceride transfer protein | ACTATCTTTATTTCC |
| | (MTTP), RefSeqGene on chromosome 4 | CTGTCGCCTCTCAGC |
| | | ACTGCAGCTTCATGT |
| SEQ ID | GI 238637280 NG_012233 Homo sapiens | TTGGTGGCTACAAGG |
| NO: 2905 | pyruvate dehyrogenase | TGACTCTGGGACAGA |
| | phosphatase catalytic subunit 1 | TGCATGGCCTTTTAA |
| | (PDP1), RefSeqGene on chromosom [symbol] | CAGAAAGGAGAACCA |
| SEQ ID | GI 893579206 NG_007504 Homo sapiens | TCTTCCACGAGGTAG |
| NO: 2906 | sonic hedgehog | TGCTGGGACTGTGAG |
| | (SHH), RefSeqGene on chromosome 7 | GGAAATTCTCATTGA |
| | | GAGTGCAGACTCCAT |
| SEQ ID | GI 50659096 NM_005218 Homo sapiens | GCCAAGTGCTGCAAG |
| NO: 2907 | defensin beta 1 | TGAGCTGGGAGTGAC |
| | (DEFB1), mRNA | CAGAAGAAATGACGC |
| | | AGAAGTGAAATGAAC |
| SEQ ID | GI 183300 M63509 Human | TCAGCTACCCACTAT |
| NO: 2908 | glutathione transferase M2 | CCTTCGTGAACATCC |
| | (GSTM2) mRNA, complete cds | CCTCCCATCATTACC |
| | | CTTCCCTGCACTAAA |
| SEQ ID | GI 15021772 AF400075 Homo sapiens | AGCTGTTTGTCTGTA |
| NO: 2909 | coagulation factor II | ACTGAGTGGGGGGAT |
| | (thrombin) receptor-like 1 | GCTGCCTTGAGCATC |
| | (F2RL1) gene, complete cds | TGTGAGTGTGTATTT |
| SEQ ID | GI 345441799 NG_029868 Homo sapiens | CATCAACATCCTCCT |
| NO: 2910 | advanced glycosylation | CACTGCTCTTCTCCC |
| | end-product specific receptor | TCCTCTGTAGACGAA |
| | (AGER), RefSeqGene on chromosome | AGCGCCGTAACTTCA |
| SEQ ID | GI 237858740 NG_012111 Homo sapiens | GAACGTTTGTTTTCC |
| NO: 2911 | platelet derived growth factor subunit B | CCTCTGTAAAATGGG |
| | (PDGFB), RefSeqGene on chromosome 22 | GGTTAGATTGGCCAT |
| | | GGGAGATGCTGGCTG |
| SEQ ID | GI 216548280 NG_009104 Homo sapiens | CCATAGAGAAGTGAC |
| NO: 2912 | retinol binding protein 4 | TGGCCTAGGGGCACA |
| | (RBP4), RefSeqGene on chromosome 10 | GGGTGAATTTTTGGC |
| | | ACTTAGTGACACCTC |
| SEQ ID | GI 219802033 NM_001143835 Homo sapiens | TGAACTCATCTTAGC |
| NO: 2913 | nuclear factor related to | CTTGTTCAGTGGGGA |
| | kappaB binding protein | GAACTTCCGCTTTGG |
| | (NFRKB), transcript variant 1, mRNA | AAACCCTCTGCACAT |
| SEQ ID | GI 21914799 AY131997 Homo sapiens | GTGGAGCTGACATCT |
| NO: 2914 | tumor necrosis factor | GAAGTCACTGCCTGT |
| | receptor superfamily, member 1A | CAGGGTAGCTATAAA |
| | (TNFRSF1A) gene, complete cds | GGAGGGAAGCAGAGT |
| SEQ ID | GI 383209671 NG_032573 Homo sapiens | GCTGCTTGTTGCCTG |
| NO: 2915 | DnaJ heat shock protein family | CTCTGTCTTGTGTGC |
| | (Hsp40) member B6 | TGTGACTGATGGGCA |
| | (DNAJB6), RefSeqGene on chromosome | GGTTTTTAGTTACTG |
| SEQ ID | GI 757813200 NG_012843 Homo sapiens | CCCCACAACTGTGAC |
| NO: 2916 | lipin 1 | AGCAGCTTCCCATGA |
| | (LPIN1), RefSeqGene on chromosome 2 | TGCCTTTTTCATCCT |
| | | CAAAGCAGATCTGTG |
| SEQ ID | GI 281332105 NG_016229 Homo sapiens | TGCAAGGGCAGCTCA |
| NO: 2917 | toll like receptor 2 | GGATCTTTAAACTCC |
| | (TLR2), RefSeqGene on chromosome 4 | ATTCCCTCAGGGCTC |
| | | ACAGAAGCTGTAAAA |
| SEQ ID | GI 428673540 NM_006273 Homo sapiens | CAAACCAGAAACCTC |
| NO: 2918 | C-C motif chemokine ligand 7 | CAATTCTCATGTGGA |
| | (CCL7), mRNA | AGCCCATGCCCTCAC |
| | | CCTCCAACATGAAAG |
| SEQ ID | GI 567316248 NM_022128 Homo sapiens | CTTGGAAGACATGCT |
| NO: 2919 | ribokinase | CAACAGATCCAATTT |
| | (RBKS), transcript variant 1, mRNA | CATTGCAGCAGTCAG |
| | | TGTCCAGGCTGCAGG |
| SEQ ID | GI 148733545 EF636667 Homo sapiens | CAGGCTCTGGTTCTC |
| NO: 2920 | caspase 4, apoptosis- | AGACTGTTCTCACAA |
| | related cysteine peptidase | TGCAGATCACCAATC |
| | (CASP4) gene, complete cds | ACTCAGGGTTGGCTT |
| SEQ ID | GI 662840 L39370 Homo sapiens | CGCCCTGCTCCGTTC |
| NO: 2921 | heat shock protein 27 | CTCCCAAAACTCTGA |
| | (HSPB1) gene, complete cds | ATCGAAGAACTTTCC |
| | | GGAAGTTTCTGAGAG |
| SEQ ID | GI 374349247 NG_032044 Homo sapiens | TCATTCGTCTTATTT |
| NO: 2922 | glutamic-oxaloacetic transaminase 1 | CTGTTCCTCTGGGAC |
| | (GOT1), RefSeqGene on chromosome 10 | AAGAGGAGTGGTGGG |
| | | CCTTCTTCCACTCCT |
| SEQ ID | GI 1024846788 NG_047009 Homo sapiens | CAGCAAGGATGATTT |
| NO: 2923 | platelet and endothelial | CTTACTTACTCTGTC |
| | cell adhesion molecule 1 | AGCCCAGCACCTGGC |
| | (PECAM1), RefSeqGene on chromosome | ACAGGTTCTCGACAC |
| SEQ ID | GI 342307092 NM_001716 Homo sapiens | CCCTAAGACGTCCCT |
| NO: 2924 | C-X-C motif chemokine receptor 5 | TTTTTCTCTGAGTAT |
| | (CXCR5), transcript variant 1, mRNA | CTCCTCGCAAGCTGG |
| | | GTAATCGATGGGGGA |
| SEQ ID | GI 32479526 NM_000218 Homo sapiens | CGGGCCACCATTAAG |
| NO: 2925 | potassium voltage-gated | GTCATTCGACGCATG |
| | channel subfamily Q member 1 | CAGTACTTTGTGGCC |
| | (KCNQ1), transcript variant 1, mRN [symbol] | AAGAAGAAATTCCAG |
| SEQ ID | GI 1049010878 AH003215 Homo sapiens | CCCTGCTTTCAAGCC |
| NO: 2926 | CD8 antigen | TCTTAGCGCACCTGG |
| | (CD8A) gene, complete cds, | AAGCGACTTAGATAC |
| | alternatively spliced | TGTTCCCATTTCACA |
| SEQ ID | GI 47419892 AY623660 Homo sapiens | GTTTCATTCAGTGTG |
| NO: 2927 | TNF receptor-associated factor 2 | AGAGTGAAGAGGCCA |
| | (TRAF2) gene, complete cds | ACGGGGCAGGTCATG |
| | | TAACCTCTGTGTCAG |
| SEQ ID | GI 347658933 NG_030027 Homo sapiens | TTCTGCTGGGGACCA |
| NO: 2928 | AT-rich interaction domain 5B | GAAAGGAGGGACAGC |
| | (ARID5B), RefSeqGene on chromosome 10 | AGGGTATAGTGAAAG |
| | | AGTGCTAAGCTTACA |
| SEQ ID | GI 225007577 NG_011607 Homo sapiens | AGAGAGAACAGCCAG |
| NO: 2929 | MER proto-oncogene, tyrosine kinase | TGCAAACCTTTGGGC |
| | (MERTK), RefSeqGene on chromosome 2 | GGAGGAGTGCTTGGT |
| | | ATATGTGAGCAATAG |
| SEQ ID | GI 334848111 NG_029036 Homo sapiens | CAATCTCAGAATCCA |
| NO: 2930 | promyelocytic leukemia | GTCCGCGTGATGATG |
| | (PML), RefSeqGene on chromosome 15 | CTCTCCTCACTGGAC |
| | | TGAGTAGGGGTCAAT |
| SEQ ID | GI 1025608669 NG_047000 Homo sapiens | GGTCCATACAGGCAC |
| NO: 2931 | prostaglandin E receptor 4 | TCAGAGGCCAAGTCA |
| | (PTGER4), RefSeqGene on chromosome 5 | CCTTCTATCTAGTGG |
| | | CTCTGCCATATACCC |
| SEQ ID | GI 632794850 NM_002957 Homo sapiens | TTGCTGTTTATCGAT |
| NO: 2932 | retinoid X receptor alpha | GCTGGTTTTCAGAAT |
| | (RXRA), transcript variant 1, mRNA | TCCTGTGTGGCCCTC |
| | | CTGTCTGGAGTGACA |
| SEQ ID | GI 116069823 EF050529 Homo sapiens | TGCATGTTTCTGAGC |
| NO: 2933 | caspase 10, apoptosis- | TCATCTTCCCAGCTC |
| | related cysteine peptidase | TCTCCAGGAGCTCCT |
| | (CASP10) gene, complete cds | ACTCCTGAATTTCCA |
| SEQ ID | GI 1036032812 AH005421 Homo sapiens | TTCAAATACTGCCCA |
| NO: 2934 | CD19 | ACCTCAAAGCCCAGC |
| | (CD19) gene, complete cds | TCCAAAGCTACCTCC |
| | | TCTGTGAAGAACTCC |
| SEQ ID | GI 4455060 AF118265 Homo sapiens | ATCCAAGTTTCAGAG |
| NO: 2935 | orphan G protein-coupled receptor GPR44 | GCAGAAAAAGGCTCG |
| | (GPR44) gene, complete cds | GGAAGGTTAAATGAC |
| | | TTGCCCAAGCACAGC |
| SEQ ID | GI 146045339 EF543149 Homo sapiens | GTTCTGTCTGAACGC |
| NO: 2936 | prostaglandin E synthase | AGAGAGTTTGTTTCG |
| | (PTGES) gene, complete cds | TCCCCTTTTCCAGTT |
| | | TCTAGAGGCCCCTGG |
| SEQ ID | GI 27501939 AY208161 Homo sapiens | TTCAGAAGGGTGTGG |
| NO: 2937 | transforming growth factor, beta 3 | AATTTGAGCAGAGGC |
| | (TGFB3) gene, complete cds | TTCAAGGATGAGGTT |
| | | TGGGGATGAAGGGAT |
| SEQ ID | GI 284520137 NG_016421 Homo sapiens | AAGGAGGGCACCTAA |
| NO: 2938 | adrenoceptor beta 2 | AGTACTTGACAGCGA |
| | (ADRB2), RefSeqGene on chromosome 5 | GTGTGCTGAGGAAAT |
| | | CAGCAGCTGTTGAAG |
| SEQ ID | GI 189339222 NG_007959 Homo sapiens | AGCACACCTTGATCA |
| NO: 2939 | cytochrome P450 family | GTGCATAAAGCCTCT |
| | 27 subfamily A member 1 | GATCCTCCTTAGAGA |
| | (CYP27A1), RefSeqGene on chromosome [symbol] | AGGAGGGACTTGGGT |
| SEQ ID | GI 574290111 NG_033893 Homo sapiens | TGTAAATTCTTTGGT |
| NO: 2940 | MALTI paracaspase | GGCATGGAACATCTG |
| | (MALT1), RefSeqGene on chromosome 18 | AGCCCTAAGGATGGC |
| | | AGACCTTCCAGGAGG |
| SEQ ID | GI 1024846756 NG_047011 Homo sapiens | AAGCCAGCAGAGGCA |
| NO: 2941 | mannose receptor C-type 1 | GGTGTCTGGATCCAG |
| | (MRC1), RefSeqGene on chromosome 10 | GCTTTACTGAGTTCC |
| | | AATTCCAAGAAATCA |
| SEQ ID | GI 1147399622 NG_012448 Homo sapiens | GCAAGTTATCTTGGA |
| NO: 2942 | nodal growth differentiation factor | AGGGGGACTGGAAGA |
| | (NODAL), RefSeqGene on chromosome 10 | ACAGTAATTCGGAGT |
| | | CTGGGCTTGGCAGTT |
| SEQ ID | GI 241896851 NG_012345 Homo sapiens | CTGAGATCCAGACTG |
| NO: 2943 | peroxisome proliferator | GTTAGAAGCCTAACA |
| | activated receptor delta | TGACCTGGCGTGTGA |
| | (PPARD), RefSeqGene on chromosome 6 | GACCTTGCACTCTCT |
| SEQ ID | GI 294459987 NG_017046 Homo sapiens | AGGAGCTCCAAGTTA |
| NO: 2944 | spleen associated tyrosine kinase | GGCAGGAAAGCCCCA |
| | (SYK), RefSeqGene on chromosome 9 | AGCTTACATTTGGAT |
| | | GGGTTGACTATGAGG |
| SEQ ID | GI 219521851 NG_009233 Homo sapiens | TGCTGAAAAGCATGG |
| NO: 2945 | tyrosine 3-monooxygenase/tryptophan | TGCTGGTAACAGTTC |
| | 5-monooxygenase activation | AACAATCCGTGGCTG |
| | protein epsilon | CTCATTCTTGCCTAC |
| | (YWHA [symbol] | |
| SEQ ID | GI 411147377 NM_014339 Homo sapiens | GTTTGACGCCCAGAG |
| NO: 2946 | interleukin 17 receptor A | TACAGGATACCACAA |
| | (IL17RA), transcript variant 1, mRNA | TGCACTCTTCCTGCG |
| | | TAGAGCACATGTTCC |
| SEQ ID | GI 336285452 NM_006221 Homo sapiens | CTGATCAACGGCTAC |
| NO: 2947 | peptidylprolyl cis/trans isomerase, | ATCCAGAAGATCAAG |
| | NIMA-interacting 1 | TCGGGAGAGGAGGAC |
| | (PIN1), transcript variant 1, mRNA | TTTGAGTCTCTGGCC |
| SEQ ID | GI 727346118 NM_002728 Homo sapiens | GAACACCTGATTACT |
| NO: 2948 | proteoglycan 2, pro eosinophil | GGGCACCTGCATAGT |
| | major basic protein | GCTGCCAGGACCTTT |
| | (PRG2), transcript variant 1, mRNA | CAAGGTTGTAGGTAG |
| SEQ ID | GI 1041818013 NM_006065 Homo sapiens | AATGGAAATGTGTCC |
| NO: 2949 | signal regulatory protein beta 1 | CGGACAGAAACAGCT |
| | (SIRPB1), transcript variant 1, mRNA | TCGACCCTCATAGAG |
| | | AACAAGGATGGCACC |
| SEQ ID | GI 269846911 NM_001167942 Homo sapiens | TAGACAGATGGGTGA |
| NO: 2950 | TNF alpha induced protein 8 like 1 | CCTGTCTCCTTTGAG |
| | (TNFAIP8L1), transcript variant 1, mRNA | AGGATGCTGAGGCAT |
| | | CTGTAGCAGCTGTTT |
| SEQ ID | ref \| NG_034014.1\| Homo sapiens | TGTTTGGGATGACCA |
| NO: 2951 | cyclin dependent kinase 2 | GGATCCCAAGCCTCC |
| | (CDK2), RefSeqGene on | TGCTGCCACAATGTT |
| | | TATAAAGGCCAAATG |
| SEQ ID | GI 219842221 NG_009291 Homo sapiens | GGACTCTTTCATCAT |
| NO: 2952 | albumin | GGAAGATCTGGGGGC |
| | (ALB), RefSeqGene on chromosome 4 | AGAGGGACTTAAAAG |
| | | GTGGGACAAGCCAAA |
| SEQ ID | GI 261823945 NG_013238 Homo sapiens | GCCCCTCCCTGGGTG |
| NO: 2953 | interleukin 9 receptor | AGGACTTTAGTATTA |
| | (IL9R), RefSeqGene on chromosome X and Y | TCATAAGACCAGGGT |
| | | CATTCTCCTGGCCTT |
| SEQ ID | GI 343403834 NG_029695 Homo sapiens | CCCTGGCATGCCTGG |
| NO: 2954 | prefoldin subunit 5 | ACCATTAACCCTTAG |
| | (PFDN5), RefSeqGene on chromosome 12 | CTCCCTTCTGTTCTT |
| | | CTCTTGTAACCAAGG |
| SEQ ID | GI 257743484 NG_012879 Homo sapiens | AAGCCTCAGCATTCA |
| NO: 2955 | serpin family A member 3 | TGAAAATCCACTACT |
| | (SERPINA3), RefSeqGene on chromosome 14 | CCAGACAGACGGCTT |
| | | TGGAATCCACCAGCT |
| SEQ ID | GI 1099522105 NG_052009 Homo sapiens | TTTTGCLLGGCAGCC |
| NO: 2956 | salt inducible kinase 1 | TCTCAGGTTGGALTT |
| | (SIK1), RefSeqGene on chromosome 21 | CTCAGGATTTGCCAT |
| | | TTGTTTTAATCCCTG |
| SEQ ID | GI 685844874 NM_002988 Homo sapiens | CCACCCTGGAGGCTA |
| NO: 2957 | C-C motif chemokine ligand 18 | CCTCTTCTAAGAGTC |
| | (CCL18), mRNA | CCATCTGCTATGCCC |
| | | AGCCACATTAACTAA |
| SEQ ID | GI 224494751 NM_138801 Homo sapiens | GAGAGCCTTGCCTTT |
| NO: 2958 | galactose mutarotase | CACAGCACAGTGAGG |
| | (GALM), mRNA | ATGACATTGCACCAG |
| | | GGACAGAGATTGTAT |
| SEQ ID | GI 55775471 NM_004271 Homo sapiens | TAGCGACAGCGGCTT |
| NO: 2959 | lymphocyte antigen 86 | GGAAGTGCTCTACCA |
| | (LY86), mRNA | GAGTTGCGATCCATT |
| | | ACAAGATTTTGGCTT |
| SEQ ID | GI 332078493 NM_002559 Homo sapiens | GCGGCCTTTACTTCT |
| NO: 2960 | purinergic receptor P2X 3 | GTGGGAGTGGGAACT |
| | (P2RX3), mRNA | GTTCTCTGTGACATC |
| | | ATCCTGCTCAACTTC |
| SEQ ID | GI 238776812 NG_012249 Homo sapiens | CCCGGGGATTCAGTA |
| NO: 2961 | aldehyde dehydrogenase 1 family member A1 | GATCTGAGGTGGGGT |
| | (ALDH1A1), RefSeqGene on chromosome 9 | CAAAGAAGCACATTT |
| | | GGCTACCAAGTGATG |
| SEQ ID | GI 237874185 NG_012167 Homo sapiens | ATCCACGTGGAACCT |
| NO: 2962 | CD209 molecule | AAGTGCTCAGACCGA |
| | (CD209), RefSeqGene on chromosome 19 | GGAACAGGGACTGAG |
| | | TTAAGAAGTGGACAC |
| SEQ ID | GI 1146072968 NG_011674 Homo sapiens | CAGGCACTGTGCTAG |
| NO: 2963 | glycine amidinotransferase | GGATATACTGGTGGG |
| | (GATM), RefSeqGene on chromosome 15 | TAAAACAGACTCCCT |
| | | GTCCTTAAGGAGCCA |
| SEQ ID | GI 429535871 NG_033022 Homo sapiens | CATCTGATGATGGCC |
| NO: 2964 | interleukin 12A | TTTGTGCCCCATCAT |
| | (IL12A), RefSeqGene on chromosome 3 | CATATGGCGGAAGGT |
| | | GAGAGAGAGAGCAAG |
| SEQ ID | GI 195546896 NG_008330 Homo sapiens | GCTGCATTCTGCACA |
| NO: 2965 | integrin subunit alpha 2 | GATGTGGTGCACTTG |
| | (ITGA2), RefSeqGene on chromosome 5 | CTCTGAGACATTTGA |
| | | GTCCCAAAGCTTAGC |
| SEQ ID | GI 1025608653 NG_047027 Homo sapiens | AACAGTGGGTGCCAA |
| NO: 2966 | Jun proto-oncogene, AP-1 | CTCATGCTAACGCAG |
| | transcription factor subunit | CAGTTGCAAACATTT |
| | (JUN), RefSeqGene on chromosome | TGAAGAGAGACCGTC |
| SEQ ID | GI 401709987 NG_032900 Homo sapiens | CCTTGCACCATGTCC |
| NO: 2967 | prostaglandin-endoperoxide synthase 1 | CTGCTTGGGTCTCAA |
| | (PTGS1), RefSeqGene on chromosome 9 | GAGCACTACTGTGAC |
| | | GGTTTTCCATCATAT |
| SEQ ID | GI 238018078 NG_012202 Homo sapiens | CAGTGGGAAGTTCTG |
| NO: 2968 | TNF receptor superfamily member 11b | CTGGGGAAAACTTCA |
| | (TNFRSF11B), RefSeqGene on chromosome 8 | GGACTCCTACCTACC |
| | | AGGATGTGACTCACT |
| SEQ ID | GI 42794755 NM_016234 Homo sapiens | GCAAGCAAGATGCCC |
| NO: 2969 | acyl-CoA synthetase long chain family member 5 | ACACAACAGGCTTAT |
| | (ACSL5), transcript variant 1, mRNA | TTTCTGTGAAGGAAC |
| | | CAACTGATCTCCCCC |
| SEQ ID | GI 36030972 NM_181713 Homo sapiens | CAAGGATCTCAAAGA |
| NO: 2970 | UBX domain protein 2A | AGTCCTCCGTTTTCC |
| | (UBXN2A), mRNA | CTGGCAACAGCTCTT |
| | | CCTGTCCTCAGGTTG |
| SEQ ID | GI 1024336747 NG_046907 Homo sapiens | AGATGTTTTGGTTGT |
| NO: 2971 | acetyl-CoA carboxylase beta | CCCAGCTGATGGAGG |
| | (ACACB), RefSeqGene on chromosome 12 | TAGATGCTACTGGCA |
| | | TCAAACAGGCAGAGG |
| SEQ ID | GI 334085206 NG_029005 Homo sapiens | CAGGAAACGGATTGA |
| NO: 2972 | CASP8 and FADD like apoptosis regulator | TGGGCTGTGATGGAG |
| | (CFLAR), RefSeqGene on chromosome 2 | GGCTTCAGTCTTATC |
| | | CAAGAGGCTTGGAAT |
| SEQ ID | GI 570359607 NG_033871 Homo sapiens | AGTATCAGGAGCCCT |
| NO: 2973 | F-box protein 38 | GAATTCTAGTTTCAC |
| | (FBX038), RefSeqGene on chromosome 5 | CCCTTCCACCTACAC |
| | | ACCGTGTGACCTTGT |
| SEQ ID | GI 296923725 NG_012838 Homo sapiens | AGTTGTGCTGACTCA |
| NO: 2974 | glucose-6-phosphate isomerase | GTCTTAGGCCTGGGG |
| | (GPI), RefSeqGene on chromosome 19 | ACAGTGGGTTAGTCA |
| | | CAAGTTGGTAGAACT |
| SEQ ID | GI 342837712 NG_029640 Homo sapiens | GGGCAGAGAAGAGTT |
| NO: 2975 | mitogen-activated protein kinase- | GTTCTTTGCAGGGAC |
| | activated protein kinase 2 | TGCAGCTTTTCTTGG |
| | (MAPKAPK2), RefSeqGene on ch [symbol] | TAATGGTTTGAGGCC |
| SEQ ID | GI 570700862 NG_033874 Homo sapiens | GAGAAAGGCAGTGTT |
| NO: 2976 | nuclear factor kappa B subunit 2 | CAGGTGTCCATGTCC |
| | (NFKB2), RefSeqGene on chromosome 10 | CCACCCAACTCTGGA |
| | | GGTAAATGACATGTC |
| SEQ ID | GI 974987494 NG_011716 Homo sapiens | AGTTCCAAGCAGGCC |
| NO: 2977 | transcription factor 4 | TCTTGCACTCCCTCC |
| | (TCF4), RefSeqGene on chromosome 18 | TTTCTTAACCCTTCC |
| | | GTAACTTTCCAACAG |
| SEQ ID | GI 238018078 NG_012202 Homo sapiens | TGGAGACATATAACT |
| NO: 2978 | TNF receptor superfamily member 11b | TGAACACTTGGCCCT |
| | (TNFRSF11B), RefSeqGene on chromosome 8 | GATGGGGAAGCAGCT |
| | | CTGCAGGGACTTTTT |
| SEQ ID | GI 61744441 NM_003262 Homo sapiens | ACACAACAGCCTTTT |
| NO: 2979 | SEC62 homolog, preprotein | TACCCTTAGGAACAG |
| | translocation factor | CTTTTTGCCCTTAGC |
| | (SEC62), mRNA | ACTATGCTTGGGGGC |
| SEQ ID | GI 46241839 AY588246 Homo sapiens | GAGCTCACATTCATA |
| NO: 2980 | epidermal growth factor receptor | GGTGCCGCCAGCCTT |
| | (erythroblastic leukemia viral | CGTGCATCTTCTTGC |
| | (v-erb-b) oncogene homolog, a [symbol] | ATCATCTCTAAGGAG |
| SEQ ID | GI 1024846791 NG_047016 Homo sapiens | AAATGGAAGGCACAA |
| NO: 2981 | bactericidal/permeability- | AAGAGATTCTTGTGC |
| | increasing protein | GTTTCCATGCTTGCG |
| | (BPI), RefSeqGene on chromosome 20 | GTTTGTTGGGTGTGC |
| SEQ ID | GI 1024846743 NG_047006 Homo sapiens | GAGCTCAGACCTGAG |
| NO: 2982 | hexokinase 3 | GAGAGTGACTAGCTT |
| | (HK3), RefSeqGene on chromosome 5 | CTCTGTGTCCCAGGT |
| | | AAGCACTTCGGTCTA |
| SEQ ID | GI 1024357482 NG_046924 Homo sapiens | ACCCAGAAGCTCAAT |
| NO: 2983 | NLR family pyrin domain containing 5 | GCACACCAAAGTCTC |
| | (NLRP5), RefSeqGene on chromosome 19 | ACATGGTTAAATTGA |
| | | GGCAAGGTGGGCTTG |
| SEQ ID | GI 1036032720 AH002608 Homo sapiens | CAGGGAGTGAGATTT |
| NO: 2984 | amphiregulin | CCCCTGTGAGTGAAA |
| | (AREG) gene, complete cds | TGCCTTCTAGTAGTG |
| | | AACCGTCCTCGGGAG |
| SEQ ID | GI 341865611 NG_029556 Homo sapiens | ATAGGCCAAGTCAGA |
| NO: 2985 | growth factor receptor bound protein 2 | TGAGGGCAGGGAATT |
| | (GRB2), RefSeqGene on chromosome 17 | GACCTTTGGATTCAG |
| | | CAACAGGAGCTGATT |
| SEQ ID | GI 231570519 NG_012004 Homo sapiens | CACCTCCTTCCTTCC |
| NO: 2986 | kinase insert domain receptor | TAACCTCACTTCTTA |
| | (KDR), RefSeqGene on chromosome 4 | CTCCCCTTTGCTCAC |
| | | TCTTACCTGGCTGCT |
| SEQ ID | GI 1068937316 NG_051332 Homo sapiens | ACCACACTCTCAGGG |
| NO: 2987 | pyruvate dehyrogenase | CTCCAAGTGGCTTTT |
| | phosphatase catalytic subunit 2 | TCTGGTTGTCTCCCA |
| | (PDP2), RefSeqGene on chromoso [symbol] | TTTTTCTCTGTCTCC |
| SEQ ID | GI 389565481 NM_002054 Homo sapiens | CTGGCAACGTTCCCT |
| NO: 2988 | glucagon | TCAAGACACAGAGGA |
| | (GCG), mRNA | GAAATCCAGATCATT |
| | | CTCAGCTTCCCAGGC |
| SEQ ID | GI 207028588 NM_021970 Homo sapiens | AGTTCAGTACATATG |
| NO: 2989 | late endosomal/lysosomal | CATTGAGCACCTGTG |
| | adaptor, MAPK and MTOR activator 3 | TGCCAGTCAGCCGAG |
| | (LAMTOR3), transcript vari [symbol] | CTATCACATTGCACA |
| SEQ ID | GI 219879808 NM_000922 Homo sapiens | ATAGTGGGTCCCCTG |
| NO: 2990 | phosphodiesterase 3B | TGTAACTCCTATGAT |
| | (PDE3B), mRNA | GCTGCTGGTTTGCTA |
| | | CCAGGTCAGTGGTTA |
| SEQ ID | GI 22001412 AF531102 Homo sapiens | CCCACTCACCTCCTT |
| NO: 2991 | interleukin 1 receptor, type 1 | ATCCAACTCTCACAC |
| | (IL1R1) gene, complete cds | ACCCAAACCAATGTT |
| | | ACCCCAGCCTCAATC |
| SEQ ID | GI 86278451 DQ374395 Homo sapiens | GGAGAAAAGACACCC |
| NO: 2992 | UDP glucuronosyltransferase 1 | CAGACTTATCCCAAT |
| | family, polypeptide A1 | GAGGAACACGGCTGT |
| | (UGT1A1) gene, complete cds | TGGGTGCTTGAGTAC |
| SEQ ID | GI 189217842 NG_007929 Homo sapiens | TGTCAGAGAACAACA |
| NO: 2993 | cytochrome P450 family 2 | TGGAGGAATGAGTGG |
| | subfamily B member 6 | GAGGAGAAGTCAGAG |
| | (CYP2B6), RefSeqGene on | CGGTAATAGGGCCAC |
| | chromosome 19 [symbol] | |
| SEQ ID | GI 761631399 NG_042041 Homo sapiens | ATGACTCCAACAGCT |
| NO: 2994 | integrin subunit beta 6 | TTGATTTCTGTTCTG |
| | (ITGB6), RefSeqGene on chromosome 2 | CTTCAGGGGCAAAGG |
| | | TCCAGATGACCCTCA |
| SEQ ID | GI 1212890194 NG_032955 Homo sapiens | AGGTCGCTGCAGGCA |
| NO: 2995 | mitogen-activated protein kinase | TACCTTGGAGATTTT |
| | 8 interacting protein 2 | GCAGGTTTGGTTCTA |
| | (MAPK8IP2), RefSeqGene on chro [symbol] | GACCACCACACTAAG |
| SEQ ID | GI 225579099 NG_011677 Homo sapiens | TTGCCCCTTCCCCAT |
| NO: 2996 | pyruvate kinase, liver and RBC | CTCCATTTCACACAG |
| | (PKLR), RefSeqGene on chromosome 1 | GTCCTGAAAGTCTGT |
| | | GTCCAATTATGCACT |
| SEQ ID | GI 91984778 NM_002214 Homo sapiens | TTGCTGCTGGTGATG |
| NO: 2997 | integrin subunit beta 8 | ACAGATCAGACGTCT |
| | (ITGB8), mRNA | CATCTCGCTCTTGAT |
| | | AGCAAATTGGCAGGC |
| SEQ ID | GI 52854078 AY755659 Homo sapiens | GCCCCCAACACATGG |
| NO: 2998 | small proline-rich protein 1A | AGCCAAGCACTTGAA |
| | (SPRRIA) gene, complete cds | CACTCACACCATACC |
| | | CACAAACACATATTC |
| SEQ ID | GI 257153342 NG_012809 Homo sapiens | GTCCGTGGAGATCAT |
| NO: 2999 | cysteinyl leukotriene receptor 1 | GAGGTCTCCTGCAGC |
| | (CYSLTR1), RefSeqGene on chromosome X | TAAGATTCCAAATGT |
| | | CCATGGCCCAAATGT |
| SEQ ID | GI 334883167 NG_029059 Homo sapiens | GTTAGACATCAAGGT |
| NO: 3000 | mitogen-activated protein kinase 9 | GGAACTGGGAATAGG |
| | (MAPK9), RefSeqGene on chromosome 5 | GAGGCAGAGGAATGA |
| | | GCCTGGAGTTCAGGA |
| SEQ ID | GI 116734711 NM_000020 Homo sapiens | CCAGCATTGTGCAAG |
| NO: 3001 | activin A receptor like type 1 | GCTCGGAAGAGAACC |
| | (ACVRL1), transcript variant 1, mRNA | AGGAAGTGAAACTGG |
| | | GTGAAAACAGAAAGC |
| SEQ ID | GI 541862110 NM_017434 Homo sapiens | TCCTGGACATCCTGG |
| NO: 3002 | dual oxidase 1 | TGGTCTTCATGAAAG |
| | (DUOX1), transcript variant 1, mRNA | GCTCTCCTGAGGAAA |
| | | AGTCTCGCCTTATGT |
| SEQ ID | GI 260763875 NM_182569 Homo sapiens | AGAAGCAGCGATTCC |
| NO: 3003 | Glycerophosphodiester | TCAGTAAACACATCT |
| | phosphodiesterase domain containing 1 | CTCACCGCGGAGGTG |
| | (GDPD1), transcript variant [symbol] | CTGGAGAAAATTTGG |
| SEQ ID | GI 315360639 NM_012474 Homo sapiens | CCTTCACACTAATGA |
| NO: 3004 | uridine-cytidine kinase 2 | GCCTTCTACTGACCC |
| | (UCK2), mRNA | AAGTTTCTTTTTCTC |
| | | GGGCAGCCTGGGACC |
| SEQ ID | GI 84872755 DQ355026 Homo sapiens | GGCTGGCTGTGCCCT |
| NO: 3005 | caspase 8, apoptosis-related | TCCGTCCTTCATTAG |
| | cysteine peptidase | ACGTTTGCTCTTGTC |
| | (CASP8) gene, complete cds | TTAGATGCTCAGATG |
| SEQ ID | GI 117606581 EF064765 Homo sapiens | TAGTTTGAGATAAGT |
| NO: 3006 | chemokine | GGGGCTGGGTGCCAG |
| | (C-C motif) ligand 21 | CAAATCCAGCTTTCA |
| | (CCL21) gene, complete cds | GGAAGGAAAAGGAGT |
| SEQ ID | GI 20336662 AF503510 Homo sapiens | AACAGCTTGCAGACT |
| NO: 3007 | coagulation factor X | CCAGTTTCGAAATCC |
| | (F10) gene, complete cds | TCTCTTTGCAGATGG |
| | | CGACCAGTGTGAGAC |
| SEQ ID | GI 1028630732 NG_050570 Homo sapiens | GCTGCCATCATTCCA |
| NO: 3008 | carnitine palmitoyltransferase 1C | GCAATCAGCAACTCC |
| | (CPT1C), RefSeqGene on chromosome 19 | ATGATGCCACACCCC |
| | | ACTATACACAACAAC |
| SEQ ID | GI 189217848 NG_007931 Homo sapiens | CGGAGGGAAGAGAGT |
| NO: 3009 | cytochrome P450 family 2 | CAGAGATCTGCCTAT |
| | subfamily J member 2 | ATCTCCTCCAAGGGG |
| | (CYP2J2), RefSeqGene on chromosome 1 | AAGTGCACCTCCTTA |
| SEQ ID | GI 401782607 NG_032906 Homo sapiens | TGATTGCGCATTACT |
| NO: 3010 | coagulation factor II thrombin receptor | CATTCCTTTCTCACA |
| | (F2R), RefSeqGene on chromosome 5 | CTTCCACCACAGAGG |
| | | CTGCCTACTTTGCCT |
| SEQ ID | GI 224586932 NG_011403 Homo sapiens | GTTCACCTGCAGCTT |
| NO: 3011 | coagulation factor VIII | CTTGTTGCTGGGGTA |
| | (F8), RefSeqGene on chromosome X | ACTTTACCCTTGTAG |
| | | GCAGCTCTAGTGGAC |
| SEQ ID | GI 368711279 NG_031983 Homo sapiens | TCTTCATGTCACCTT |
| NO: 3012 | heat shock protein family B | CTTGACAGCATTTGC |
| | (small) member 6 | CAGGCATCCAGCAGG |
| | (HSPB6), RefSeqGene on chromosome 19 | CGCTTAATAAATGGC |
| SEQ ID | GI 270483830 NG_016181 Homo sapiens | AAGCTCCTAAAGGGT |
| NO: 3013 | sphingosine-1-phosphate receptor 1 | TCATTTGGCCCCTCC |
| | (SIPR1), RefSeqGene on chromosome 1 | TCAAAGACTAATGTC |
| | | CCCATGTGAAAGCGT |
| SEQ ID | GI 30841527 AY277591 Homo sapiens | TGGCATTTATCTTTG |
| NO: 3014 | GalNAc-T5 | TAGCTTCTGTCATCT |
| | (GALNT5) mRNA, complete cds | GGCTCCTCTTTGACA |
| | | TGGCAGCTCTCCGCC |
| SEQ ID | GI 327412387 NG_028268 Homo sapiens | CACTCCCTGAAAACT |
| NO: 3015 | ATP binding cassette | CACTCTTGCTTTCAG |
| | subfamily C member 1 | ATTTGAGGGCCACTT |
| | (ABCC1), RefSeqGene on chromosome 16 | CCACCTCACTAGGCC |
| SEQ ID | GI 1025608659 NG_047042 Homo sapiens | ATTGGCCAAGCCTGG |
| NO: 3016 | cell division cycle 42 | GTCACGTGTCCATTC |
| | (CDC42), RefSeqGene on chromosome 1 | CCGCAACTAGAGATA |
| | | GTGTCATCTCTAGAG |
| SEQ ID | GI 268370133 NG_015859 Homo sapiens | AGGAGGTAGAAAGGA |
| NO: 3017 | GATA binding protein 3 | ACGGAAAAGTGCTCC |
| | (GATA3), RefSeqGene on chromosome 10 | AGGTGTGTATCTCAG |
| | | CTTCCTGTGCTCTGT |
| SEQ ID | GI 742670592 NG_013347 Homo sapiens | CTGCACCTCCCTCAG |
| NO: 3018 | glutamate-ammonia ligase | TGAGGACTGTAGGAA |
| | (GLUL), RefSeqGene on chromosome 1 | TTTCAGTATTTTAGA |
| | | GATGGTGGCCCCGGA |
| SEQ ID | GI 297515475 NG_021404 Homo sapiens | GCCTGGTTTGTCACA |
| NO: 3019 | matrix metallopeptidase 13 | GCTGAGATACAGCTG |
| | (MMP13), RefSeqGene on chromosome 11 | CACCATGGCAAACTG |
| | | GTGAAGCAACATTAG |
| SEQ ID | GI 431822400 NM_001271969 Homo sapiens | TTTTCCCTCTCCTGT |
| NO: 3020 | heat shock protein 90 alpha | CCTTGTGTTGAAGGC |
| | family class B member 1 | AGTAAACTAAGGGTG |
| | (HSP90AB1), transcript | TCAAGCCCCATTCCC |
| | variant 1, [symbol] | |
| SEQ ID | GI 20379137 AF498982 Homo sapiens | TGCAAAGAGTCCTGC |
| NO: 3021 | 5-hydroxytryptamine receptor 2A | AATGAGGATGTCATT |
| | (HTR2A) mRNA, complete cds | GGGGCCCTGCTCAAT |
| | | GTGTTTGTTTGGATC |
| SEQ ID | GI 599044742 NM_012234 Homo sapiens | TGCACCTTCAGAAAC |
| NO: 3022 | RING1 and YYl binding protein | AGTGCTGAAGCCTTT |
| | (RYBP), mRNA | AAATGCAGCATCTGC |
| | | GATGTGAGGAAAGGC |
| SEQ ID | GI 58258007 AY902236 Homo sapiens | GCCTTACCTGCTCTG |
| NO: 3023 | flavin containing monooxygenase 5 | GGGAATTGCAGGTTG |
| | (FMO5) gene, complete cds | CCACATAACAAAGCA |
| | | TTTCTGTGTGGGTAC |
| SEQ ID | GI 14488405 AF386077 Homo sapiens | TCTACATGTCCACAG |
| NO: 3024 | interleukin 17B | GTCCAGTTCACCCAC |
| | (IL17B) gene, complete cds | TTCCTTGTCCCGTAT |
| | | TTAGCAGATGGGGAA |
| SEQ ID | GI 38045925 AY449709 Homo sapiens | GGCAAGTGCGGCTGT |
| NO: 3025 | interleukin 7 receptor | CCTATGTCTCCTTTT |
| | (IL7R) gene, complete cds | CCAGAAGGATGAAGA |
| | | TGTCTTTGGGACTGG |
| SEQ ID | GI 161086962 NG_007114 Homo sapiens | CCCCAGAGACCTTGT |
| NO: 3026 | insulin | TCAGGAAAGGGAATG |
| | (INS), RefSeqGene on chromosome 11 | AGAACATTCCAGCAA |
| | | TTTTCCCCCCACCTA |
| SEQ ID | GI 342307076 NG_029608 Homo sapiens | AGATGACATTTAGTC |
| NO: 3027 | phospholipase D2 | TAAGTCTGTGGCAAG |
| | (PLD2), RefSeqGene on chromosome 17 | CAGCAAAGCAGGGCA |
| | | TATGTGGCAGAGCCT |
| SEQ ID | GI 429535866 NG_033016 Homo sapiens | TGGAAAGTAGAAGGG |
| NO: 3028 | receptor interacting | GAGCGAGCTATGCAG |
| | serine/threonine kinase 2 | AGAAATCACATGGTG |
| | (RIPK2), RefSeqGene on chromosome 8 | AGACAGGATGCAGGA |
| SEQ ID | GI 1154491910 NG_052982 Homo sapiens | ACTTGTTGGCCTTGT |
| NO: 3029 | ubiquitin conjugating enzyme E2 G2 | GTTCTTTCCGACCCA |
| | (UBE2G2), RefSeqGene on chromosome 21 | AGACATGGCTTAGTA |
| | | AGTGAGCAGTGTGGT |
| SEQ ID | GI 112293258 NM_001736 Homo sapiens | CCCCTGATTATGGGC |
| NO: 3030 | complement C5a receptor 1 | ACTATGATGACAAGG |
| | (C5AR1), mRNA | ATACCCTGGACCTCA |
| | | ACACCCCTGTGGATA |
| SEQ ID | GI 572873826 NM_001130080 Homo sapiens | AGCCATCCTGACCCA |
| NO: 3031 | interferon alpha inducible protein 27 | GCGAGGAGCCAACTA |
| | (IFI27), transcript variant 1, mRNA | TCCCAAATATACCTG |
| | | GGGTGAAATATACCA |
| SEQ ID | GI 523498473 NM_006332 Homo sapiens | TGAAGAAGTCCAATG |
| NO: 3032 | IFI30, lysosomal thiol reductase | CACCGCTTGTCAATG |
| | (IFI30), mRNA | TGACCCTCTACTATG |
| | | AAGCACTGTGCGGTG |
| SEQ ID | GI 32891808 AY341028 Homo sapiens | GGAAGCTTCAATATC |
| NO: 3033 | microsomal glutathione S-transferase 2 | AAAGGAGCAGGGGAT |
| | (MGST2) gene, complete cds | TGTTCAAAGAGGGCT |
| | | CTCGCCAGGGAATGA |
| SEQ ID | GI 169234676 NG_007118 Homo sapiens | CTGGAATAACCACAG |
| NO: 3034 | ATP binding cassette | GAATGTTGGTTGGCT |
| | subfamily B member 4 | TGGAATGGATGAAGG |
| | (ABCB4), RefSeqGene on chromosome 7 | GAAATGGAACCGGGC |
| SEQ ID | GI 219521908 NG_009245 Homo sapiens | ATAGGCTGGATCCTG |
| NO: 3035 | bone morphogenetic protein | CTCACTGCATCTTAT |
| | receptor type 1B | TACTCCGGTTTCCCC |
| | (BMPR1B), RefSeqGene on chromosome 4 | TTTGCTTTCAGCAGC |
| SEQ ID | GI 916259104 NG_042801 Homo sapiens | AAATGGGAAGCCAGT |
| NO: 3036 | bromodomain containing 2 | TCTGCAAATTGGCCT |
| | (BRD2), RefSeqGene on chromosome 6 | TTCTGTTCTGTGAAG |
| | | TGCCATCTGTCGGTG |
| SEQ ID | GI 357197116 NG_030591 Homo sapiens | GGGTAGACTTATCAA |
| NO: 3037 | cholecystokinin B receptor | GGCACTTGTGGATGT |
| | (CCKBR), RefSeqGene on chromosome 11 | ACCCCAGCAGGTGCT |
| | | ATCTCCCATCCTTCT |
| SEQ ID | GI 291575141 NG_016810 Homo sapiens | AGTTAACCGAGAGAG |
| NO: 3038 | nuclear receptor coactivator 3 | CGAGCATCTACCTCA |
| | (NCOA3), RefSeqGene on chromosome 20 | CTGGCAGGATCTGGG |
| | | ATTCCCAAATGGAAT |
| SEQ ID | GI 215598755 NG_009072 Homo sapiens | TCTTTCCCCAGCCCT |
| NO: 3039 | von Willebrand factor | TCCAGTGATTAAAAG |
| | (VWF), RefSeqGene on chromosome 12 | CCATGCAGACGGATA |
| | | AGCAGTCTGTGATGG |
| SEQ ID | GI 284172478 NM_052946 Homo sapiens | GGGCAAACCTGGAAA |
| NO: 3040 | nitric oxide synthase trafficking | TTAGCTATGCCAAAG |
| | (NOSTRIN), transcript variant 1, mRNA | GACTTCAGAAACTGG |
| | | CAAGCAAGCTGAGCA |
| SEQ ID | GI 748585188 NM_004040 Homo sapiens | CTGTACAGAGAATAC |
| NO: 3041 | ras homolog family member B | ACCTGCCCCTGTATA |
| | (RHOB), mRNA | TCLTTTTTTCCCCTC |
| | | CCCTLLCTCCCAGTG |
| SEQ ID | GI 32187524 AY324387 Homo sapiens | TGGAGGACCTCCGCT |
| NO: 3042 | glutathione S-transferase pi | GCAAATACATCTCCC |
| | (GSTP1) gene, complete cds | TCATCTACACCAACT |
| | | ATGTGAGCATCTGCA |
| SEQ ID | GI 27501935 AY207429 Homo sapiens | ACTCCAAAGACCCTG |
| NO: 3043 | interleukin 11 | GATCTCAAATTCCAA |
| | (ILH) gene, complete cds | CTTCTAGCTCTGAGA |
| | | CTCCAGCCCTCACCC |
| SEQ ID | GI 21914605 AY130302 Homo sapiens | CAAATGCGAAAGAGT |
| NO: 3044 | truncated recombination | GGGAGATGTAAGAGG |
| | activating gene 1 | AGCGTCGAATGAGGG |
| | (RAG1) gene, complete cds | CAGCACAGAACAGTG |
| SEQ ID | GI 228008299 NG_011964 Homo sapiens | TAAGGTCAGTGGGCC |
| NO: 3045 | arginase 2 | AAAACGAAAAGAAAG |
| | (ARG2), RefSeqGene on chromosome 14 | GTGAATGGCTTGCAG |
| | | GGTTTTGCTGGAGTT |
| SEQ ID | GI 283945491 NG_016362 Homo sapiens | CTTGAAGGATGAGCT |
| NO: 3046 | C-X3-C motif chemokine receptor 1 | GACATCTCGCTCAGG |
| | (CX3CR1), RefSeqGene on chromosome 3 | GCCAAATAACCGACT |
| | | TGCTTACTGCTTGCT |
| SEQ ID | GI 193794846 NG_008174 Homo sapiens | GAGAGGTGTCGGTGG |
| NO: 3047 | fructose-bisphosphatase 1 | GCCTTTTTTCTTTTC |
| | (FBP1), RefSeqGene on chromosome 9 | AACTCTTGTGTCAAC |
| | | GTCAAGCCCCTCCTA |
| SEQ ID | GI 594591631 NG_034042 Homo sapiens | TCCATCAGAAGCTGT |
| NO: 3048 | mitogen-activated protein kinase 11 | AGGTGGGTCATTCTA |
| | (MAPK11), RefSeqGene on chromosome 22 | GCCATCATGCCAGCA |
| | | CAGATCTGTCTTCCT |
| SEQ ID | GI 1024336758 NG_046923 Homo sapiens | CAGTGCAGGCACCCT |
| NO: 3049 | zinc fingers and homeoboxes 3 | GGGAGCTAAATGTTA |
| | (ZHX3), RefSeqGene on chromosome 20 | CTGTATGTACCAGCA |
| | | GGGCTTTGTGAGAAA |
| SEQ ID | GI 1134787083 NM_004833 Homo sapiens | CTTCTGTGGCTGCTA |
| NO: 3050 | absent in melanoma 2 | GTGAGAACCCAAACC |
| | (AIM2), transcript variant 1, mRNA | AGCTCAGCCAATTAG |
| | | AGCTCCAGTTGTCAC |
| SEQ ID | GI 10798850 AF084559 Homo sapiens | AGGGTGTCCTGAGGT |
| NO: 3051 | fatty acid desaturase 2 | CCAAGATTCTGGAGC |
| | (FADS2) mRNA, complete cds | AATCTGACCCTTCTC |
| | | CAAAGGCTCTGTTAT |
| SEQ ID | GI 296841050 NM_138733 Homo sapiens | AATTGTGAAAGCCAC |
| NO: 3052 | phosphoglycerate kinase 2 | TTCCAAGGGCTGCAT |
| | (PGK2), mRNA | CACTGTTATAGGGGG |
| | | TGGAGACACTGCTAC |
| SEQ ID | GI 109809760 NM_014445 Homo sapiens | GCCCCCGAAGAGAAG |
| NO: 3053 | stress associated | GCGTCTGTAGGACCC |
| | endoplasmic reticulum protein 1 | TGGTTATTGGCTCTC |
| | (SERP1), mRNA | TTCATTTTTGTTGTC |
| SEQ ID | GI 27357272 AY195619 Homo sapiens | GAATCCTTCAGGGCA |
| NO: 3054 | interleukin 10 receptor, alpha | TGGTTTGGAAGGCCA |
| | (IL10RA) gene, complete cds | GGTAGGGATTCGCAG |
| | | AAACCTAGACACATC |
| SEQ ID | GI 13447389 AF361105 Homo sapiens | CAGACTTACAAGCCT |
| NO: 3055 | interleukin 9 | TGAAGCCGGGAAGCA |
| | (IL9) gene, complete cds | CCCGTTGAAAAGAAC |
| | | ATTCAGAGCCGACTA |
| SEQ ID | GI 342672038 NG_029624 Homo sapiens | TGAAATCCTTTTGGA |
| NO: 3056 | NFKB inhibitor beta | TGGTTTTCCCATCCC |
| | (NFKBIB), RefSeqGene on chromosome 19 | CACAGACTCACAGGC |
| | | ATGGCTGATCCGTAT |
| SEQ ID | GI 574957093 NM_001252 Homo sapiens | CACCAACCTCACTGG |
| NO: 3057 | CD70 molecule | GACACTTTTGCCTTC |
| | (CD70), transcript variant 1, mRNA | CCGAAACACTGATGA |
| | | GACCTTCTTTGGAGT |
| SEQ ID | GI 130509310 NM_022073 Homo sapiens | AACAAGAATTGGGAT |
| NO: 3058 | egl-9 family hypoxia inducible factor 3 | GCCAAGCTACATGGT |
| | (EGLN3), transcript variant 1, mRNA | GGGATCCTGCGGATA |
| | | TTTCCAGAGGGGAAA |
| SEQ ID | GI 194018480 NM_001945 Homo sapiens | CTTGGTAACTGATTA |
| NO: 3059 | heparin binding EGF like growth factor | CCTGCCAATTGCTAC |
| | (HBEGF), mRNA | CGAGAAGGTTGGAGG |
| | | TGGGGAAGGCTTTGT |
| SEQ ID | GI 666637974 NM_005922 Homo sapiens | GGAGTACTGCGATGA |
| NO: 3060 | mitogen-activated protein | GGGGACTTTAGAAGA |
| | kinase kinase kinase 4 | GGTGTCAAGGCTGGG |
| | (MAP3K4), transcript variant 1, mRNA | ACTTCAGGAACATGT |
| SEQ ID | GI 11342663 NM_002463 Homo sapiens | AGAGAAGAATCCTTA |
| NO: 3061 | MX dynamin like GTPase 2 | AGGAGAGAATTTACC |
| | (MX2), mRNA | GGCTCACTCAGGCGC |
| | | GACACGCACTCTGTC |
| SEQ ID | GI 42794610 NM_012088 Homo sapiens | CCCTCACACTACCTG |
| NO: 3062 | 6-phosphogluconolactonase | TCCTGAATGCAGCAC |
| | (PGLS), mRNA | GAACTGTCATCTTTG |
| | | TGGCAACTGGAGAAG |
| SEQ ID | GI 27368251 AY196903 Homo sapiens | TGGTGGCAGCCAGTA |
| NO: 3063 | interleukin 1 receptor antagonist | GGCTGGTCTGCTCCT |
| | (ILIRN) gene, complete cds | TCCTCTCTATTTCAT |
| | | ATGTGTATGAAGGCA |
| SEQ ID | GI 209447058 NG_008718 Homo sapiens | AGCTTGAGTCAGGAC |
| NO: 3064 | apurinic/apyrimidinic | CCTTAATTAAGATCC |
| | endodeoxyribonuclease 1 | TCAATTGGCTGGAGG |
| | (APEX1), RefSeqGene on chromosome 14 | GCAGATCTCGCGAGT |
| SEQ ID | GI 341865533 NG_029560 Homo sapiens | GCCAGAAAACAGGGA |
| NO: 3065 | aquaporin 4 | AAGAGGCTGAGAAAC |
| | (AQP4), RefSeqGene on chromosome 18 | CGTGGTAACCAAGTT |
| | | TTGCTGGCACTTTGT |
| SEQ ID | GI 269954682 NG_015989 Homo sapiens | TCTCTGGAATTGTTT |
| NO: 3066 | C-C motif chemokine ligand 26 | TCAGGGCCGTCTCAG |
| | (CCL26), RefSeqGene on chromosome 7 | TCTCATAAAAGGGGA |
| | | TCAGGCAGGAGGAGT |
| SEQ ID | GI 345441766 NG_029866 Homo sapiens | GTTACTTGTCCTGAT |
| NO: 3067 | DnaJ heat shock protein family | CACTCAGCCTCACTC |
| | (Hsp40) member A3 | AGCTGTTCCCTCACT |
| | (DNAJA3), RefSeqGene on chromosome [symbol] | CAGCCAGGATGTCTT |
| SEQ ID | GI 723428434 NG_041793 Homo sapiens | AGTAGGACCCAGGCA |
| NO: 3068 | inhibitor of nuclear factor | GTAGGTTTTACTGAG |
| | kappa B kinase subunit beta | GGAACGGACATCCTG |
| | (IKBKB), RefSeqGene on chromosome [symbol] | TTGCCTTTCTCACTC |
| SEQ ID | GI 215820636 NG_009079 Homo sapiens | CCACAACACAGAGAT |
| NO: 3069 | malonyl-CoA decarboxylase | GGGCTTGATCTGTCG |
| | (MLYCD), RefSeqGene on chromosome 16 | CACATTGGAGGCCTG |
| | | GGATTTATCTTCTCC |
| SEQ ID | GI 289802979 NG_016616 Homo sapiens | GGTTTCCAGGGGGCG |
| NO: 3070 | phosphorylase kinase | TGTGACTTGTCAAAG |
| | catalytic subunit gamma 2 | ACTAAGGATTTACAA |
| | (PHKG2), RefSeqGene on chromosome 16 | GGGGACAGGCAGAAA |
| SEQ ID | GI 340138935 NG_029446 Homo sapiens | TGAATCCTATCCTTA |
| NO: 3071 | PYD and CARD domain containing | GGAATCACCAAACAA |
| | (PYCARD), RefSeqGene on chromosome 16 | CCACTTTCCAACCTC |
| | | ACTGCCCCACCAGGC |
| SEQ ID | GI 332800978 NM_001206950 Homo sapiens | AGGAGGTACAGAAGC |
| NO: 3072 | solute carrier family 16 member 3 | CGGCAACGCTTGCTA |
| | (SLC16A3), transcript variant 1, mRNA | TTTATTTTACAAACT |
| | | GGACTGGCTCAGGCA |
| SEQ ID | GI 209977034 NM_021995 Homo sapiens | ATGCTCTGCGTCACT |
| NO: 3073 | urotensin 2 | TCTGCTCGGACTCAT |
| | (UTS2), transcript variant 1, mRNA | AAATCCACGTCTCTT |
| | | TGCTTTGGCCACTTC |
| SEQ ID | GI 114731580 DQ993361 Homo sapiens | AGAAAAGAGTGCTCC |
| NO: 3074 | glutathione S-transferase A3 | AAATAGGAGAAACAG |
| | (GSTA3) gene, complete cds | CATGCAGAGCCTTGG |
| | | GCTAGGGAGAAAGGC |
| SEQ ID | GI 410991940 NG_032977 Homo sapiens | AGGAGGGAAAGAAGG |
| NO: 3075 | interleukin 12 receptor subunit beta 2 | ACATGAGGCAATTCT |
| | (IL12RB2), RefSeqGene on chromosome 1 | TCAGAGCCGAGCTGT |
| | | CGTTAAGAGGGTCTT |
| SEQ ID | GI 335892864 NG_029105 Homo sapiens | ACACCCTGAAACAAG |
| NO: 3076 | interleukin 1 receptor accessory protein | TCCGAGTCAGGCCAG |
| | (ILIRAP), RefSeqGene on chromosome 3 | ACAAGCATCCCCTGA |
| | | AAAATACTCCCTTTG |
| SEQ ID | GI 33333853 AF546700 Homo sapiens | TCGCCACATCCTTAT |
| NO: 3077 | androgen regulated a/b hydrolase II | GGGCACCGGAAGTTC |
| | (ABHD2) mRNA, complete cds | ATCACTATGTCTGAT |
| | | GGAGCCACTTCTACA |
| SEQ ID | GI 31321969 AY114157 Homo sapiens | CCAGTTGCAGGTGGC |
| NO: 3078 | inhibitor of kappaB kinase gamma | CTATCACCAGCTCTT |
| | (IKBKG) mRNA, complete cds | CCAAGAATACGACAA |
| | | CCACATCAAGAGCAG |
| SEQ ID | GI 1036029324 AHO13155 Homo sapiens | AATGTCTTTTCCAGG |
| NO: 3079 | chromosome 19 poliovirus | TGGCTTTTTTGGAAT |
| | receptor related 2 | CAGGATGCTGCGAAG |
| | (PVRL2) gene, | CTGCCTACGTTGCAT |
| SEQ ID | GI 1168024008 KY497359 Homo sapiens | CAGCCCCACACCAAG |
| NO: 3080 | HLA-E | AGTTTTTGGAGGTCT |
| | (HLA-E) gene, complete cds | GACTCCAGCTTTTCT |
| | | CAGTCACTCAGCATC |
| SEQ ID | GI 302129699 NG_012063 Homo sapiens | TTTCATTTCATTCCT |
| NO: 3081 | enolase 3 | TGGTCGGGGAGGGGG |
| | (ENO3), RefSeqGene on chromosome 17 | GATAAGGCTACAAAG |
| | | GGGAGAGGAGAGAAT |
| SEQ ID | GI 194595503 NG_008308 Homo sapiens | ACTAGTCCCTGGGCT |
| NO: 3082 | gap junction protein alpha 1 | GTCATCAAATGTACC |
| | (GJA1), RefSeqGene on chromosome 6 | CATGATACCGTGGTA |
| | | CCATCTAGCTGGTGC |
| SEQ ID | GI 237858739 NG_012110 Homo sapiens | AGGTCCCATGGAAAA |
| NO: 3083 | programmed cell death 1 | TGACCACAGGCAATG |
| | (PDCD1), RefSeqGene on chromosome 2 | AGAAAGCCATAGAGT |
| | | GCTGCCTGTGAGATC |
| SEQ ID | GI 300796511 NG_023374 Homo sapiens | GATGGGCTGTCTCCC |
| NO: 3084 | retinoid X receptor beta | CATTTCTAACCTGAG |
| | (RXRB), RefSeqGene on chromosome 6 | TGGTTCCTCCTCTGA |
| | | TTCCATGGACCTTCT |
| SEQ ID | GI 226693341 NG_011851 Homo sapiens | CCTGACCTTACTTTC |
| NO: 3085 | synuclein alpha | CTCCCTCCCTGTTTC |
| | (SNCA), RefSeqGene on chromosome 4 | CTCTTTGTGGTGTTT |
| | | CCACTGGCCAAACTC |
| SEQ ID | GI 260656026 NM_005518 Homo sapiens | TAGAACACAGCTTCC |
| NO: 3086 | 3-hydroxy-3-methylglutaryl-CoA synthase 2 | CCCAGCCCACTTCCC |
| | (HMGCS2), transcript variant 1, mRNA | TGTTCTAAAGACACC |
| | | ACATAGACAGAGAGC |
| SEQ ID | GI 1049010946 AH002554 Homo sapiens | TGTTGAACCTTTTTA |
| NO: 3087 | actin prepeptide | GGGGGTGATGGTGGG |
| | (ACTA2) gene, complete cds | AATGGGACAAAAAGA |
| | | CAGCTACGTGGGTCA |
| SEQ ID | GI 167887711 EF445033 Homo sapiens | AGGCATGCTGATTCT |
| NO: 3088 | signal transducing adapter molecule 1 | CAGTCCAGTGTTCTT |
| | (STAM) gene, complete cds, | TTCGCTGCCCGAGTC |
| | alternatively spliced | TGCCTTCTTTGTTAG |
| SEQ ID | GI 1150273800 NG_052917 Homo sapiens | CATTTCAGGAAATGA |
| NO: 3089 | adenosine A1 receptor | ACAGGAAGTGCAAAG |
| | (ADORAl), RefSeqGene on chromosome 1 | CCCCTGAGGTAGGAG |
| | | AGTGCCCCAGGTATG |
| SEQ ID | GI 269784608 NG_015894 Homo sapiens | TTGAGACATATGTAT |
| NO: 3090 | apolipoprotein A5 | CCAACTGCCAGAACA |
| | (APOA5), RefSeqGene on chromosome 11 | TCTCCAGTGGACAGC |
| | | CTTTGGGCACAAGGC |
| SEQ ID | GI 906847382 NG_042271 Homo sapiens | GGTTTTGTGCTGCTT |
| NO: 3091 | calcium voltage- | TCTGTCTTTTTTGGA |
| | gated channel subunit alphal B | AGTCGGGAGGGGCAC |
| | (CACNA1B), RefSeqGene on chromosome 9 | ACAAACCAGAAGGAA |
| SEQ ID | GI 224809239 NG_011444 Homo sapiens | GCTGCCTAGAGGCTG |
| NO: 3092 | fatty acid binding protein 2 | ACTCAACTGAAATCA |
| | (FABP2), RefSeqGene on chromosome 4 | TGGCGTTTGACAGCA |
| | | CTTGGAAGGTAGACC |
| SEQ ID | GI 1216866539 NG_012872 Homo sapiens | TGTAAACTGGGAGTG |
| NO: 3093 | gelsolin | GCGGTACCTCCCTTG |
| | (GSN), RefSeqGene on chromosome 9 | CAGATACACTCACCT |
| | | TGAAAGCTGCAAAAC |
| SEQ ID | GI 345091009 NG_029843 Homo sapiens | GGCAAGTGGTGTGGT |
| NO: 3094 | nuclear receptor subfamily 1 | TCTAGCCGTGAAGAC |
| | group H member 4 | ACAAAAGCTTCCAAA |
| | (NR1H4), RefSeqGene on chromosome 12 | ACCAAAGTCAGGCAG |
| SEQ ID | GI 206725550 NM_004958 Homo sapiens | CCAAGAGTGGTGCAG |
| NO: 3095 | mechanistic target of rapamycin | GAAGTACATGAAATC |
| | (MTOR), mRNA | AGGGAATGTCAAGGA |
| | | CCTCACCCAAGCCTG |
| SEQ ID | GI 31559181 AY310878 Homo sapiens | AATCAGCCTCTCCTT |
| NO: 3096 | glutathione peroxidase 3 | GGAGTCAGTCTAAAT |
| | (plasma) | GGATGTGAAGCCACT |
| | (GPX3) gene, complete cds | TCGTCCAAGTCCCCT |
| SEQ ID | GI 51988875 AY744675 Homo sapiens | GTCAGGACTGAAAAG |
| NO: 3097 | matrix metalloproteinase 10 | GGCTCATATTCTAGA |
| | (stromelysin 2) | AGGGGCAGCAGAGAT |
| | (MMP10) gene, complete cds | GAAGCCTTTGGCAGC |
| SEQ ID | GI 284520137 NG_016421 Homo sapiens | ATTTTCCTAAGCATC |
| NO: 3098 | adrenoceptor beta 2 | CAGCTATTGGTGTGA |
| | (ADRB2), RefSeqGene on chromosome 5 | GGGCATGCTCTGGCA |
| | | CCATGGAAGCTTACT |
| SEQ ID | GI 237858769 NG_012123 Homo sapiens | AGACCCAGAATCTGG |
| NO: 3099 | C-C motif chemokine ligand 2 | TCTGTGCTTCATTCA |
| | (CCL2), RefSeqGene on chromosome 17 | CCTTAGCTTCCAGAG |
| | | ACGGTGACTCTGCAG |
| SEQ ID | GI 297307113 NG_021395 Homo sapiens | GCAGGGCTGGGATGC |
| NO: 3100 | erythropoietin receptor | ATGCCAGACTAGACC |
| | (EPOR), RefSeqGene on chromosome 19 | CAGACTCTTCGTTAA |
| | | CATTTTCTGCTCTTG |
| SEQ ID | GI 381140032 NG_032168 Homo sapiens | AGTTGCTGGTTGGGA |
| NO: 3101 | glycerol-3-phosphate dehydrogenase 1 | TTATTGGGCTTCACC |
| | (GPD1), RefSeqGene on chromosome 12 | ACTGGATCTTAAGAG |
| | | CCCTTTCCCGACTTG |
| SEQ ID | GI 1063759771 NG_051548 Homo sapiens | ATAGTTTCACTGCCC |
| NO: 3102 | mitogen-activated protein | CCTGGGTTCTCCCAA |
| | kinase-activated protein kinase 3 | CGCTGCTCCTTACCT |
| | (MAPKAPK3), RefSeqGene on [symbol] | AGAAGGAAAGAATCT |
| SEQ ID | GI 291219867 NG_016781 Homo sapiens | TTGCTGCTGCCTATT |
| NO: 3103 | pyruvate dehydrogenase alpha 1 | GTAGATCTGGGAAGG |
| | (PDHA1), RefSeqGene on chromosome X | TAAGGCTCTAAAGCC |
| | | CTCTGGGCTAGTGAC |
| SEQ ID | GI 345842406 NM_005849 Homo sapiens | CTGCTCAGCAAGGAA |
| NO: 3104 | immunoglobulin superfamily member 6 | CTGCGGAGCTTCCTG |
| | (IGSF6), mRNA | ACAGCTCTTGTATCA |
| | | CTGCTCTCTGTCTAT |
| SEQ ID | GI 411147377 NM_014339 Homo sapiens | CCAATGAACGTTTGT |
| NO: 3105 | interleukin 17 receptor A | GCGTCAGGTTTGAGT |
| | (IL17RA), transcript variant 1, mRNA | TTCTGTCCAAACTGA |
| | | GGCATCACCACAGGC |
| SEQ ID | GI 1150273800 NG_052917 Homo sapiens | GGTACCTGGAAGCCA |
| NO: 3106 | adenosine A1 receptor | AGTGTAGACAGTCTA |
| | (ADORAl), RefSeqGene on chromosome 1 | CCAAGGAGGGTGGAC |
| | | AAATGGGTGAAGTGA |
| SEQ ID | GI 269954733 NG_016014 Homo sapiens | TCTTAATGATGCCCC |
| NO: 3107 | membrane spanning 4-domains A2 | TGTGTCTAGGGTATG |
| | (MS4A2), RefSeqGene on chromosome 11 | CCAGGTCCTGTCTGC |
| | | CATTGCCTGAGATAG |
| SEQ ID | GI 38261972 NM_024551 Homo sapiens | CCCCGGAATGTACAG |
| NO: 3108 | adiponectin receptor 2 | TGTCTTGGTGCACCA |
| | (ADIPOR2), mRNA | AGATGCCTTCTAAAG |
| | | GCTGACATACCTTGG |
| SEQ ID | GI 197304767 NM_014674 Homo sapiens | ACCCAGACCATGCCA |
| NO: 3109 | ER degradation enhancing | AAGTCCAGTCTGAAA |
| | alpha-mannosidase like protein 1 | TGAAAGGGGACAGAA |
| | (EDEM1), mRNA | GTCTTGCTGTCCATG |
| SEQ ID | GI 523498705 NM_024923 Homo sapiens | TCCTTGAGATGGCAG |
| NO: 3110 | nucleoporin 210 | CAGGAGTCATATTTT |
| | (NUP210), mRNA | CTGTGTGTGTGTTTC |
| | | GGAAAGCCGCTGTGT |
| SEQ ID | GI 1036032346 AH001409 Homo sapiens | GGGTGTATTGGCTTG |
| NO: 3111 | alcohol dehydrogenase 6 | GGAGTAGAAGAAGGG |
| | (ADH6) gene, complete cds | TCGTCCCTGCTGCAG |
| | | AAGCTGTATACTGAT |
| SEQ ID | GI 189409117 NG_007972 Homo sapiens | AATCATTCCTGCCCA |
| NO: 3112 | cytochrome P450 family | CCAGTATGCCGTGTC |
| | 2 subfamily C member 8 | AATTTACTATTCCCA |
| | (CYP2C8), RefSeqGene | TGGCAACACCCAGGA |
| | on chromosome 10 [symbol] | |
| SEQ ID | GI 197116377 NG_008385 Homo sapiens | GGGGCAATCTAGGAT |
| NO: 3113 | cytochrome P450 family 2 | AATACAGCAGCCCCA |
| | subfamily C member 9 | AAGCAGCAGAGAGTC |
| | (CYP2C9), RefSeqGene on | AAGTGCCTTCCTTTT |
| | chromosome 10 [symbol] | |
| SEQ ID | GI 347658934 NG_030028 Homo sapiens | CCCCAGAGGAAGTAA |
| NO: 3114 | mitochondrial antiviral signaling protein | GGTCTTCATCATCCA |
| | (MAVS), RefSeqGene on chromosome 20 | GTGGCCTGGACTCAA |
| | | CTCCAGATGTCAGTG |
| SEQ ID | GI 1026191100 NG_047066 Homo sapiens | GCAAGGAGGCTTTTC |
| NO: 3115 | proliferating cell nuclear antigen | CTCCCGGTAGGCTTG |
| | (PCNA), RefSeqGene on chromosome 20 | ATTTGCATTTCACTT |
| | | TCACTTTCGTGGCTG |
| SEQ ID | GI 218749810 NG_009188 Homo sapiens | CTCTTCAGCATCCTT |
| NO: 3116 | phosphoglycerate dehydrogenase | GAACCACTTGCTGGT |
| | (PHGDH), RefSeqGene on chromosome 1 | GGTACTTACATGGTT |
| | | ACATGTGGCCGGGAC |
| SEQ ID | GI 429535866 NG_033016 Homo sapiens | ACAGGGATGACAGTG |
| NO: 3117 | receptor interacting | TGGGTAGACCAAGGC |
| | serine/threonine kinase 2 | AGACAGACTTCATAG |
| | (RIPK2), RefSeqGene on chromosome 8 | GACATTGCACCAGAA |
| SEQ ID | GI 356461041 NM_014329 Homo sapiens | GCTGTGGAGGGCAGC |
| NO: 3118 | enhancer of mRNA decapping 4 | ATGAAAGAGAACATC |
| | (EDC4), mRNA | TCCAAGCTGCTCAAG |
| | | TCCAAGAACTTGACT |
| SEQ ID | GI 62122910 NM_001014336 Homo sapiens | TACAGTCCCTCTCGA |
| NO: 3119 | interleukin 31 | AGATGCTTTTGAAAG |
| | (IL31), mRNA | ATGTGGAGGAAGAGA |
| | | AGGGCGTGCTCGTGT |
| SEQ ID | GI 315434201 NM_020429 Homo sapiens | AATCCCCCATTCCAA |
| NO: 3120 | SMAD specific E3 ubiquitin | AAGTCCAGGCCAAAT |
| | protein ligase 1 | GGTGGGCTTAGCCTC |
| | (SMURF1), transcript variant 1, mRNA | TTTGAAAAGTTCTGC |
| SEQ ID | GI 1049010967 AH003492 Homo sapiens | AGGTTGTAGGGTTTC |
| NO: 3121 | beta-fibrinogen | TTCAGTGCCCGTGGT |
| | (FGB) gene, complete cds | CATCGACCCCTTGAC |
| | | AAGAAGAGAGAAGAG |
| SEQ ID | GI 14423570 AF387519 Homo sapiens | AGGGAAACACTTGCA |
| NO: 3122 | interleukin 22 | TCTCAAGGTGGAAAG |
| | (IL22) gene, complete cds | GATAGAGGTGGTGTT |
| | | TTGTGGGCTCCTGTG |
| SEQ ID | GI 169234676 NG_007118 Homo sapiens | TCTGCCTGGTTTCTG |
| NO: 3123 | ATP binding cassette | ACCCTTTGTCTCTTT |
| | subfamily B member 4 | CGGTTCTCTTGCTTG |
| | (ABCB4), RefSeqGene on chromosome 7 | CCCTGTGTGTTTGAT |
| SEQ ID | GI 353703739 NG_030372 Homo sapiens | TGCCTGTCTGTTACA |
| NO: 3124 | CD83 molecule | CTGGAGGAGAGAAGA |
| | (CD83), RefSeqGene on chromosome 6 | ATGAGCCTACGCTGA |
| | | AGATGGCATCCTGTG |
| SEQ ID | GI 1025608669 NG_047000 Homo sapiens | CGTGGTCAGACAACT |
| NO: 3125 | prostaglandin E receptor 4 | GAAAATCTGGAGAAG |
| | (PTGER4), RefSeqGene on chromosome 5 | GTCCATTCCAGGCAA |
| | | AGGGAAGAACCTGGG |
| SEQ ID | GI 312261284 NM_016441 Homo sapiens | ACAAGGCTGCACCTT |
| NO: 3126 | cysteine rich transmembrane | GAACTGTCCCTTCGG |
| | BMP regulator 1 | TTTCCTTACTGATGC |
| | (CRIM1), mRNA | CCAAAACTGTGAGAT |
| SEQ ID | GI 325651826 NM_001443 Homo sapiens | AGCTCTATTGCCACC |
| NO: 3127 | fatty acid binding protein 1 | ATGAGTTTCTCCGGC |
| | (FABP1), mRNA | AAGTACCAACTGCAG |
| | | AGCCAGGAAAACTTT |
| SEQ ID | GI 1036031503 AHO11685 Homo sapiens | AGAGATCTCTTCGAT |
| NO: 3128 | chromosome 1 AMPK beta-2 subunit | TTCTTTCAGGCCTGA |
| | (PRKAB2) gene, complete cds | GGCTTTTGTGAAATG |
| | | CGTCAGCCCCCTGTG |
| SEQ ID | GI 1220516002 NM_023003 Homo sapiens | TGGGCACATGATCTG |
| NO: 3129 | transmembrane 6 superfamily member 1 | CTACTGGGATGGCTC |
| | (TM6SF1), transcript variant 1, mRNA | TGCTCATTATCTGAT |
| | | GTACCTGGTGATGGT |
| SEQ ID | GI 124001557 NM_152586 Homo sapiens | CACACTACCAGAAAG |
| NO: 3130 | ubiquitin specific peptidase 54 | AAGCAAGGGCCTTCA |
| | (USP54), transcript variant 1, mRNA | GGTTCCTCACACTCA |
| | | GTCCTGGAGTGATCT |
| SEQ ID | GI 21105788 AF512686 Homo sapiens | TCCTTCAATGCATGG |
| NO: 3131 | interleukin 12B | CCCATAACCATTAAA |
| | (natural killer cell stimulatory | GCAGAGGTCTTCTCA |
| | factor 2, cytotoxic lymphocyte | CTGTGCGGTCCCATC |
| | maturation factor [symbol] | |
| SEQ ID | GI 577019603 NG_033914 Homo sapiens | AAGGAGGATCAAGAC |
| NO: 3132 | CD207 molecule | CCGTAATCCGGCATA |
| | (CD207), RefSeqGene on chromosome 2 | ACACCCTGGGGTTTT |
| | | CCACTCTTTCAGAGA |
| SEQ ID | GI 1042161084 NG_046982 Homo sapiens | TGCTCAGCTACTGGC |
| NO: 3133 | ficolin 1 | ACGACTTCTAAGCTC |
| | (FCN1), RefSeqGene on chromosome 9 | TGTGTCCCTCCACCT |
| | | TCTCACCTGAAAATT |
| SEQ ID | GI 345525392 NG_029921 Homo sapiens | AGGGGGAAGTGAGAA |
| NO: 3134 | major histocompatibility | CGTGACAGAGAAGCA |
| | complex, class II, DR beta 1 | AGTGCTGGGTTTGGT |
| | (HLA-DRB1), RefSeqGene on chromoso [symbol] | GGAGTTGATATTTGC |
| SEQ ID | GI 338827642 NG_029226 Homo sapiens | TCCATCTACCTACTC |
| NO: 3135 | nuclear factor of activated T-cells 1 | TCCATCTACCCATCA |
| | (NFATC1), RefSeqGene on chromosome 18 | TCCCTTCACCCCCCG |
| | | TCCATCATGATCCAT |
| SEQ ID | GI 318064850 NG_028074 Homo sapiens | AAAGCCACTGAAATT |
| NO: 3136 | ras related dexamethasone induced 1 | TGATCTCCTCCTTCA |
| | (RASD1), RefSeqGene on chromosome 17 | CACATTCCCAGGTCC |
| | | CCAATATGCCACCCA |
| SEQ ID | GI 83267865 NM_001037494 Homo sapiens | AAGGAATTTGACAAG |
| NO: 3137 | dynein light chain LC8-type 1 | AAGTACAATCCCACC |
| | (DYNLL1), transcript variant 1, mRNA | TGGCATTGCATCGTG |
| | | GGGAGGAACTTCGGT |
| SEQ ID | GI 20379137 AF498982 Homo sapiens | CTTTGGGCTACAGGA |
| NO: 3138 | 5-hydroxytryptamine receptor 2A | CGATTCGAAGGTCTT |
| | (HTR2A) mRNA, complete cds | TAAGGAGGGGAGTTG |
| | | CTTACTCGCCGATGA |
| SEQ ID | GI 169790829 NM_001754 Homo sapiens | GCGTGGTAAAAGAAA |
| NO: 3139 | runt related transcription factor 1 | TCATTGAGTCCCCCG |
| | (RUNX1), transcript variant 1, mRNA | CCTTCAGAAGAGGGT |
| | | GCATTTTCAGGAGGA |
| SEQ ID | GI 18765742 NM_003098 Homo sapiens | AACTTCAGCGAGGCC |
| NO: 3140 | syntrophin alpha 1 | AAACACATGTCCTTG |
| | (SNTA1), mRNA | AAGATGGCATATGTC |
| | | TCGAAGAGGTGCACC |
| SEQ ID | GI 72255578 NM_021103 Homo sapiens | CCCCCCCCCAATCGG |
| NO: 3141 | thymosin beta 10 | ACTGCCAAATTCTCC |
| | (TMSB10), mRNA | GGTTTGCCCCGGGAT |
| | | ATTATAGAAAATTAT |
| SEQ ID | GI 170560906 EU567146 Homo sapiens | GGGAGATGATCATGG |
| NO: 3142 | solute carrier family 22 | GAGGAACCATGTGAA |
| | (organic anion transporter), member 6 | AGGAGTTGGGGGGAA |
| | (SLC22A6) gene, complete cds | GGCAATTTAAGGGTT |
| SEQ ID | GI 569026730 NG_012887 Homo sapiens | ATTTACAGCCAAGGA |
| NO: 3143 | cystatin C | ACATGGCGTGGAGGG |
| | (CST3), RefSeqGene on chromosome 20 | GTGGTGTTAGCAGGT |
| | | GGAAAGTTACTAAGC |
| SEQ ID | GI 374088072 NG_032029 Homo sapiens | TTTGGAGTGTTTCCC |
| NO: 3144 | MAP kinase interacting | TGATCCAGAGCTGGG |
| | serine/threonine kinase 1 | AGGTCCAGAACAGAG |
| | (MKNK1), RefSeqGene on chromosome 1 | GGTTAAGGATGGAAA |
| SEQ ID | GI 209447066 NG_008725 Homo sapiens | GTGCAACACATATGA |
| NO: 3145 | paraoxonase 2 | GCATGAAGGGGCCCA |
| | (PON2), RefSeqGene on chromosome 7 | TGCCAAATGTATAGT |
| | | GCAGCAGGAGCAAAG |
| SEQ ID | GI 302058260 NG_023447 Homo sapiens | CCGCCACCAAAATGT |
| NO: 3146 | resistin | AGATTTTCCTGAAAA |
| | (RETN), RefSeqGene on chromosome 19 | CCCTTACAGGGAAGA |
| | | CCACCACCCTCGAGG |
| SEQ ID | GI 1154345445 NG_033859 Homo sapiens | ATCCTTGCTCCAAGA |
| NO: 3147 | sodium voltage-gated | GACTCAGTACAAACC |
| | channel alpha subunit 11 | TCAAACACAAAGCTG |
| | (SCN11A), RefSeqGene on chromosome 3 | TGCTCTGCATGGCCA |
| SEQ ID | GI 212549746 NG_008982 Homo sapiens | GTTTGTCCTTGTAGG |
| NO: 3148 | solute carrier family 22 member 5 | CATGGGCCAGATCTC |
| | (SLC22A5), RefSeqGene on chromosome 5 | CAACTATGTGGCAGC |
| | | ATTTGTCCTGGGTAT |
| SEQ ID | GI 254039651 NG_012413 Homo sapiens | ACTTTGCCGTTGAAG |
| NO: 3149 | vimentin | CTGCTAACTACCAAG |
| | (VIM), RefSeqGene on chromosome 10 | ACACTATTGGCCGCC |
| | | TGCAGGATGAGATTC |
| SEQ ID | GI 66933013 NM_020139 Homo sapiens | TAGGGCCCACTTCCT |
| NO: 3150 | 3-hydroxybutyrate dehydrogenase 2 | GGTTTGCAGATGGTC |
| | (BDH2), mRNA | CTCTTCTTGCTGTAT |
| | | CCTCACATAGCAAGA |
| SEQ ID | GI 874507471 NM_005928 Homo sapiens | TCCTCACTGTCCTGT |
| NO: 3151 | milk fat globule-EGF factor 8 protein | TTTCTTAGGCACTGA |
| | (MFGE8), transcript variant 1, mRNA | GGGATCTGAGTAGGT |
| | | CTGGGATGGACAGGA |
| SEQ ID | GI 195963400 NM_005359 Homo sapiens | TCCCCTAAATTCAGA |
| NO: 3152 | SMAD family member 4 | GGAATGCAGCTATGC |
| | (SMAD4), mRNA | CAGAAGCCAGAGAAG |
| | | AGCCACTCGTAGCTT |
| SEQ ID | GI 193788644 NG_008170 Homo sapiens | CATTGGCAACCCAGC |
| NO: 3153 | branched chain amino acid transaminase 1 | ACCCAGTTAAATTCC |
| | (BCAT1), RefSeqGene on chromosome 12 | CCTTGTCACTGTTTC |
| | | CACATTGTCCCCATC |
| SEQ ID | GI 1024846747 NG_047015 Homo sapiens | AGCTGGAGCCCAGCT |
| NO: 3154 | bone gamma-carboxyglutamate protein | CTGCTTGAACCTATT |
| | (BGLAP), RefSeqGene on chromosome 1 | TTAGGTCTCTGATCC |
| | | CCGCTTCCTCTTTAG |
| SEQ ID | GI 747813982 NG_007933 Homo sapiens | GAGATGTCGAGGAGC |
| NO: 3155 | cytochrome P450 family | CAGAAGAGGTTCAGG |
| | 2 subfamily S member 1 | AGTTGCTGATTGCAG |
| | (CYP2S1), RefSeqGene | TATGAGGCACAGTGA |
| | on chromosome 19 [symbol] | |
| SEQ ID | GI 291084846 NG_016762 Homo sapiens | AAACATCCAGCTTCT |
| NO: 3156 | pyruvate dehydrogenase kinase 3 | TCCTGCTCACTGACA |
| | (PDK3), RefSeqGene on chromosome X | ACTGCCACCAATTAT |
| | | CGCTCCACTTCCAGG |
| SEQ ID | GI 315434256 NM_001926 Homo sapiens | GCAAGCTCAAGTCTT |
| NO: 3157 | defensin alpha 6 | AGAGCTTTGGGCTCA |
| | (DEFA6), mRNA | ACAAGGGCTTTCACT |
| | | TGCCATTGCAGAAGG |
| SEQ ID | GI 545746351 NM_006228 Homo sapiens | CATGAAAGTCCTGCT |
| NO: 3158 | prepronociceptin | TTGTGACCTGCTGCT |
| | (PNOC), transcript variant 1, mRNA | GCTCAGTCTCTTCTC |
| | | CAGTGTGTTCAGCAG |
| SEQ ID | GI 8133099 AF266756 Homo sapiens | GGCAGCTTCCTTGAA |
| NO: 3159 | sphingosine kinase | CCATTATGCTGGCTA |
| | (SPHK1) mRNA, complete cds | TGAGCAGGTCACCAA |
| | | TGAAGACCTCCTGAC |
| SEQ ID | GI 350606318 NG_009014 Homo sapiens | CCTGCCTGATCTGTG |
| NO: 3160 | androgen receptor | GAGATGAAGCTTCTG |
| | (AR), RefSeqGene on chromosome X | GGTGTCACTATGGAG |
| | | CTCTCACATGTGGAA |
| SEQ ID | GI 224809308 NG_011502 Homo sapiens | CCAGGAGACCCACAG |
| NO: 3161 | cytotoxic T-lymphocyte | GTATGACCTTCTAGG |
| | associated protein 4 | AAGCTCCAGTTCGAT |
| | (CTLA4), RefSeqGene on chromosome 2 | GGGCCCAATTCTTAC |
| SEQ ID | GI 1043261406 NG_051107 Homo sapiens | AAACTGTTTGAGCAG |
| NO: 3162 | vacuole membrane protein 1 | GAGTGAGTCACAGTG |
| | (VMP1), RefSeqGene on chromosome 17 | AAACCAGCTTCCCTG |
| | | TAACCACAGGGCTTG |
| SEQ ID | GI 211971007 NM_001136538 Homo sapiens | GTCAGGGTGGACTCA |
| NO: 3163 | acyl-CoA dehydrogenase family member 10 | ATCTTTCTGGTTCTC |
| | (ACAD10), transcript variant 1, mRNA | CACAGAAGACGTCTC |
| | | TGCAAGAAGCCTGGA |
| SEQ ID | GI 169234615 NM_002514 Homo sapiens | GTGATGGTCATTGGG |
| NO: 3164 | nephroblastoma overexpressed | ACCTGCACCTGTCAC |
| | (NOV), mRNA | ACCAACTGTCCTAAG |
| | | AACAATGAGGCCTTC |
| SEQ ID | GI 45934429 AY585238 Homo sapiens | CGCAGTTGCAGTCAG |
| NO: 3165 | creatine kinase, muscle | ACACAGACAAGGAGA |
| | (CKM) gene, complete cds | GTGGGAGAAAGGGAG |
| | | ACAGGAAGAGAAACA |
| SEQ ID | GI 7637751 AF250378 Homo sapiens | TACCTTTAATGGCTC |
| NO: 3166 | E2F transcription factor 4 | CTTGAGGGCCACCAG |
| | (E2F4) gene, complete cds | ATAAGATCTGAACTC |
| | | CTGAGCCTGGCATGT |
| SEQ ID | GI 308522736 NG_027815 Homo sapiens | AGGGTAGACAGGCAA |
| NO: 3167 | transketolase | TAACCACCTAATGTC |
| | (TKT), RefSeqGene on chromosome 3 | AGGTGGCAAGCATGA |
| | | GTGAGGGGAAGAAGA |
| SEQ ID | GI 574289185 NG_033890 Homo sapiens | CTACCCTCAAGTTCT |
| NO: 3168 | transient receptor potential | ACAGGTTCACCAGAT |
| | cation channel subfamily A member 1 | GCCCCAGGAGGTCCT |
| | (TRPAl), RefSeqGene on [symbol] | AGATGCATTGATGGG |
| SEQ ID | GI 88999590 NM_197947 Homo sapiens | TCATGCTCAACAATG |
| NO: 3169 | C-type lectin domain containing 7A | CCATTGAAGTGCACG |
| | (CLEC7A), transcript variant 1, mRNA | GTGTGTTGCCACGAT |
| | | TTGACCCTCAACTTC |
| SEQ ID | GI 225579058 NM_197965 Homo sapiens | ACCTGCCCAGCCATT |
| NO: 3170 | solute carrier family 10 member 6 | GTTGCCGATGTTCAT |
| | (SLC10A6), mRNA | TTGTAATGCTGCCTT |
| | | AAGGAGATGAGGAGA |
| SEQ ID | GI 315365058 HQ38416O Homo sapiens | TTTTGGGAAGCCCTC |
| NO: 3171 | fatty acid binding protein 4, adipocyte | AGGACACTTACACAG |
| | (FABP4) gene, complete cds | GCTTGTGAATCAATC |
| | | ACATGCTCAGCAGGT |
| SEQ ID | GI 8101110 AF268380 Homo sapiens | CCACCCCCACTGTGT |
| NO: 3172 | melanoma-associated delayed | TTGGCGCTTAATAGG |
| | early response protein | TTGGAGAAGAGGACC |
| | (NAB2) gene, complete cds | TGAGCAACTGAAATC |
| SEQ ID | GI 37953284 AY438977 Homo sapiens | AAAACAGGGTCCACC |
| NO: 3173 | phospholipase A2, group IB | TTGGCTCAGTCCTCT |
| | (pancreas) | CCTTTCATCCCTCTC |
| | (PLA2G1B) gene, complete cds | CAGGCAAAAACAAAG |
| SEQ ID | GI 255652920 NG_012644 Homo sapiens | TCACAGGTGCTTCCC |
| NO: 3174 | deleted in malignant brain tumors 1 | CAAACTTGAGCCTTC |
| | (DMBT1), RefSeqGene on chromosome 10 | ATAAACCCAGGCAGA |
| | | ACTGCTTCTTTGACT |
| SEQ ID | GI 257743484 NG_012879 Homo sapiens | GTCTGTGGAAGCTGA |
| NO: 3175 | serpin family A member 3 | GGGAGAGCACCAAAT |
| | (SERPINA3), RefSeqGene on chromosome 14 | TCAGTTTGGGAAGTC |
| | | AGGGAAGGCTTCTTG |
| SEQ ID | GI 215277008 NM_000851 Homo sapiens | CTTAGTGGATGGGTG |
| NO: 3176 | glutathione S-transferase mu 5 | TGTGTGTGTGTGTGT |
| | (GSTM5), mRNA | TGGGGGTGGTGATCA |
| | | GGCAGGTTCATAAAT |
| SEQ ID | GI 59853224 NM_001206 Homo sapiens | TTGCTGGGACTGTAG |
| NO: 3177 | Kruppel like factor 9 | CGTTTGCAGGAAGTC |
| | (KLF9), mRNA | ACTTAACTGTTTGGG |
| | | AGCTGGAAAACCGAA |
| SEQ ID | GI 1159261125 NM_001349413 Homo sapiens | AATGAAATGATGTCC |
| NO: 3178 | kelch like family member 24 | CCAAGGACTAGGCCA |
| | (KLHL24), transcript variant 1, mRNA | CGCAGGTCCACTGGC |
| | | TATTCTGAGGTGATA |
| SEQ ID | GI 207028588 NM_021970 Homo sapiens | ACAATGCTCCAGAGC |
| NO: 3179 | late endosomal/lysosomal adaptor, | ATGCTTTGCGACCTG |
| | MAPK and MTOR activator 3 | GTTTCTTATCCACTT |
| | (LAMTOR3), transcript vari [symbol] | TTGCCCTTGCAACAG |
| SEQ ID | GI 299758475 NM_001190737 Homo sapiens | AATCCCGACCAGAAG |
| NO: 3180 | nuclear factor 1 B | GGTAAGATTAGGAGA |
| | (NFIB), transcript variant 1, mRNA | ATCGACTGCCTGCGA |
| | | CAGGCAGACAAAGTC |
| SEQ ID | GI 13430063 AF253322 Homo sapiens | GAACACGTGACTTCT |
| NO: 3181 | cytochrome P450 | CACAAGGCCTTGATT |
| | (CYP1A1) and cytochrome P450 | TCTTCATTCACAGCC |
| | (CYP1A2) genes, complete cds | CGGGAGGGGATAAGC |
| SEQ ID | GI 117606493 EF064721 Homo sapiens | AATACACAGAGATAC |
| NO: 3182 | interleukin 7 | GGGAGTCTTGGCAGA |
| | (IL7) gene, complete cds | GAAGCTGCTCAGGCA |
| | | CATGTTGGAGAACCA |
| SEQ ID | GI 226492652 NG_011814 Homo sapiens | CCGTCTTTTTTCTCC |
| NO: 3183 | C-X-C motif chemokine receptor 1 | TGTGTCCCCAGTGAC |
| | (CXCR1), RefSeqGene on chromosome 2 | TATTTTCTGCCCTTG |
| | | GGGCTGATTTTCCCT |
| SEQ ID | GI 594591642 NG_034033 Homo sapiens | AAAGGGGAGAAATTA |
| NO: 3184 | phosphofructokinase, liver type | GGGTTTTCCGGAACC |
| | (PFKL), RefSeqGene on chromosome 21 | CCCACACATTTGTGG |
| | | TTGGGAAAGACTCCT |
| SEQ ID | GI 298160932 NG_021435 Homo sapiens | AATTTTCCATCCTCT |
| NO: 3185 | vesicle associated membrane protein 7 | GACCCCCTCACTCTG |
| | (VAMP7), RefSeqGene on chromosome X and Y | TTCATTGGCCACAAA |
| | | TCCCCACTGGTCTTG |
| SEQ ID | GI 574957130 NM_001815 Homo sapiens | GCTTCTACTTGTCCA |
| NO: 3186 | carcinoembryonic antigen | CAATCTGCCCCAGCA |
| | related cell adhesion molecule 3 | TCTTTTTGGCTACAG |
| | (CEACAM3), transcript variant 1 [symbol] | CTGGTACAAAGGGGA |
| SEQ ID | GI 221316649 NM_005232 Homo sapiens | TGGGTGGAGTGAACA |
| NO: 3187 | EPH receptor A1 | GCAACAGATACTGAA |
| | (EPHAl), mRNA | TGGGACACCCCTGTA |
| | | CATGTACCAGGACTG |
| SEQ ID | GI 385137136 NM_012340 Homo sapiens | CTTTTGAAGGCAAGG |
| NO: 3188 | nuclear factor of activated T-cells 2 | AGTTCTCAGAGCAAC |
| | (NFATC2), transcript variant 1, mRNA | CAAAGGAACGTGACC |
| | | CAAGAGCCCAGCTTA |
| SEQ ID | GI 769468216 NM_003246 Homo sapiens | TGTGGACCAGAGAGA |
| NO: 3189 | thrombospondin 1 | CACTGATATGGATGG |
| | (THBS1), mRNA | GGTTGGAGATCAGTG |
| | | TGACAATTGCCCCTT |
| SEQ ID | GI 32815083 AY340073 Homo sapiens | GTGAGACCTGAGCAC |
| NO: 3190 | glutamate decarboxylase 2 | ATCACCTGACTTCCC |
| | (pancreatic islets and brain, 65 kDa) | TGACTGTTTCCCAGT |
| | (GAD2) gene, complete cds | TTCGCAACAGCAATT |
| SEQ ID | GI 190194409 NG_008004 Homo sapiens | TAATGGGTAGTGGGG |
| NO: 3191 | activin A receptor type 1 | ACTGAGGAACTATGG |
| | (ACVR1), RefSeqGene on chromosome 2 | AATGATGGTGAGAGC |
| | | CAAGGGGGCAATGAA |
| SEQ ID | GI 662236863 NG_034192 Homo sapiens | GTAGGATTGTCTGCA |
| NO: 3192 | diacylglycerol O-acyltransferase 1 | GTGAGCTTTGGGAAG |
| | (DGAT1), RefSeqGene on chromosome 8 | CACCACCCTTTCCCG |
| | | ACTCCTGTTTTCATC |
| SEQ ID | GI 1024336756 NG_046908 Homo sapiens | TTCACCTGGGGCTCT |
| NO: 3193 | YYl transcription factor | GCCAGACGGTTACCA |
| | (YYl), RefSeqGene on chromosome 14 | CATTACCAGGACTCT |
| | | TGGTACAAATGAGAA |
| SEQ ID | GI 342307092 NM_001716 Homo sapiens | AGGGCTCACCGTGGC |
| NO: 3194 | C-X-C motif chemokine receptor 5 | TAAGAGTGTCCTAGG |
| | (CXCR5), transcript variant 1, mRNA | AGTATCCTCATTTGG |
| | | GGTAGCTAGAGGAAC |
| SEQ ID | GI 188497624 NM_012205 Homo sapiens | CATCATCCAGGAGTT |
| NO: 3195 | 3-hydroxyanthranilate 3,4-dioxygenase | CTTCAGCTCTGAGCA |
| | (HAAQ), mRNA | GTACAGAACAGGAAA |
| | | GCCCATCCCTGACCA |
| SEQ ID | GI 38016923 NM_006253 Homo sapiens | ACTGAGGCCACACAT |
| NO: 3196 | protein kinase AMP-activated | CATTTCCAGTCATCT |
| | non-catalytic subunit beta 1 | GTGTGTTTTTAAGGC |
| | (PRKAB1), mRNA | CAGCCACTTGTCCCT |
| SEQ ID | GI 21397157 AF517525 Homo sapiens | AGTCTCTTGGCCCTA |
| NO: 3197 | cyclin D3 | GTTGTGTTCCCATAC |
| | (CCND3) gene, complete cds | TAGGGAGTGTCTTGT |
| | | CTATTCCCGGGACCT |
| SEQ ID | GI 51537346 AY728138 Homo sapiens | ATCTGTGACCGCTTC |
| NO: 3198 | chemokine | TACCCCAATGACTTG |
| | (C-X-C motif) receptor 4 | TGGGTGGTTGTGTTC |
| | (CXCR4) gene, complete cds | CAGTTTCAGCACATC |
| SEQ ID | GI 32140476 AY316305 Homo sapiens | ACGCAGTCTTCTCCA |
| NO: 3199 | glutathione transferase zeta 1 | CTAGTGTTGACCCAC |
| | (maleylacetoacetate isomerase) | CCAAGTTTTTTTTAC |
| | (GSTZ1) gene, complete cds | CCTCAGCTTGCCAGG |
| SEQ ID | GI 238776831 NG_012261 Homo sapiens | AAGCATCCAGTGGAA |
| NO: 3200 | aldehyde dehydrogenase 8 family member A1 | TCCCACCTGGTGTTT |
| | (ALDH8A1), RefSeqGene on chromosome 6 | ACTGTGGGCAGAGGT |
| | | GAATCTGTGGTTTTT |
| SEQ ID | GI 297515475 NG_021404 Homo sapiens | TGAGAGGCTCCGAGA |
| NO: 3201 | matrix metallopeptidase 13 | AATGCAGTCTTTCTT |
| | (MMP13), RefSeqGene on chromosome 11 | CGGCTTAGAGGTGAC |
| | | TGGCAAACTTGACGA |
| SEQ ID | GI 291084780 NG_016747 Homo sapiens | TGCCTAATGCTTGTA |
| NO: 3202 | PPARG coactivator 1 beta | TGTGTGGAAGGCAAG |
| | (PPARGC1B), RefSeqGene on chromosome 5 | GTGCTCACTGTTCTG |
| | | GAGGCAGCCTATTTC |
| SEQ ID | GI 299829203 NG_023197 Homo sapiens | TCAGGAAACTAACAA |
| NO: 3203 | parathyroid hormone like hormone | GGTGGAGACGTACAA |
| | (PTHLH), RefSeqGene on chromosome 12 | AGAGCAGCCGCTCAA |
| | | GACACCTGGGAAGAA |
| SEQ ID | GI 229577384 NG_011990 Homo sapiens | GAGGAATTGTGGGAG |
| NO: 3204 | SMAD family member 3 | GAAAAGCACTGGGCA |
| | (SMAD3), RefSeqGene on chromosome 15 | GAAGGTCTCAGTCAA |
| | | ATGCACCTGCAGTTA |
| SEQ ID | GI 17059625 AJ420108 Homo sapiens | TCTGTGCTGGTCGGG |
| NO: 3205 | mRNA for F-box only 32 | AACATTAACATGTGG |
| | (FBXO32 gene) | GTGTATCGGATGGAG |
| | | ACGATTCTCCACTGG |
| SEQ ID | GI 206597516 NM_001859 Homo sapiens | GGCGTGGCCTTATCG |
| NO: 3206 | solute carrier family 31 member 1 | ATTGCAGTGGGAAGT |
| | (SLC31A1), mRNA | TGTTGAAGACTTGAA |
| | | GACGTGATTCCTGCT |
| SEQ ID | GI 117606493 EF064721 Homo sapiens | CAAAGACGTGCCCAC |
| NO: 3207 | interleukin 7 | TGGGTGAATGGGTAT |
| | (IL7) gene, complete cds | GTCGACATGGTCCTA |
| | | GTCTGAGTGTAGGTG |
| SEQ ID | GI 237820638 NG_012034 Homo sapiens | AAAGAATTTCAGGCC |
| NO: 3208 | ABL proto-oncogene 1, non- | GAGGGAACCAGGATG |
| | receptor tyrosine kinase | CAGAAACCTGAGGCA |
| | (ABL1), RefSeqGene on chromosome 9 | GAGCTTGCTAGTTTC |
| SEQ ID | GI 238018065 NG_012195 Homo sapiens | TGTGCCAAAGAATCT |
| NO: 3209 | fatty acid amide hydrolase | CACGATTGCTGCCTA |
| | (FAAH), RefSeqGene on chromosome 1 | TTGGAATGCGAAAAC |
| | | GCAAGCGTGACAACC |
| SEQ ID | GI 307133693 NG_027728 Homo sapiens | TGTCAACTGGCCCTG |
| NO: 3210 | interferon regulatory factor 4 | TGTAAAGCCACGTCA |
| | (IRF4), RefSeqGene on chromosome 6 | CTGTTTCTGTAGAGA |
| | | GCATGACACGTCAGA |
| SEQ ID | GI 692314725 NM_002416 Homo sapiens | TAGTGAGAAAGGGTC |
| NO: 3211 | C-X-C motif chemokine ligand 9 | GCTGTTCCTGCATCA |
| | (CXCL9), mRNA | GCACCAACCAAGGGA |
| | | CTATCCACCTACAAT |
| SEQ ID | GI 299758401 NM_000143 Homo sapiens | GTGTGACAGAACGCA |
| NO: 3212 | fumarate hydratase | TGCCAACCCCAGTTA |
| | (FH), mRNA | TTAAAGCTTTTGGCA |
| | | TCTTGAAGCGAGCGG |
| SEQ ID | GI 153082754 NM_001547 Homo sapiens | AACAAGCAGATGAAG |
| NO: 3213 | interferon induced protein with | ACTCTGAGAGGGGTT |
| | tetratricopeptide repeats 2 | TGGAGTCTGGAAGCC |
| | (IFIT2), mRNA | TCATCCCTTCAGCAT |
| SEQ ID | GI 117606541 EF064745 Homo sapiens | TTCAAATGCCTCTTC |
| NO: 3214 | chemokine | CCTCCTAGGCAGGAT |
| | (C-X3-C motif) ligand 1 | GACTCAGTACCTTTC |
| | (CX3CL1) gene, complete cds | AGCAACTGCTCTCGG |
| SEQ ID | GI 7417326 AF237581 Homo sapiens | AGTCACAGGGTCATG |
| NO: 3215 | migration inhibitory factor- | GAGGGAATAAACTGG |
| | related protein 14 variant P | AGAGCGTTTGGTATG |
| | (S100A9) gene, complete cds | TGCTCAGTGTCTGCT |
| SEQ ID | GI 751130526 NG_041830 Homo sapiens | AGTTCCTCTTGAGGT |
| NO: 3216 | DnaJ heat shock protein family | GTTTGACTGTTGGCC |
| | (Hsp40) member C3 | AAAGTCTTCCCTTGG |
| | (DNAJC3), RefSeqGene on chromosome | TTGCTTCTCACACGG |
| SEQ ID | GI 350606382 NG_030335 Homo sapiens | CGGTTTGAGAAAAGA |
| NO: 3217 | SEL1L ERAD E3 ligase adaptor subunit | AAGGGCAGGCACACC |
| | (SELIL), RefSeqGene on chromosome 14 | AAGTCACTGCTCTGC |
| | | TACTCAGTTGGAACA |
| SEQ ID | GI 305410826 NG_027551 Homo sapiens | TTTCCAGTGTCCCTG |
| NO: 3218 | transient receptor potential | GGTCACTCTCTTGGT |
| | cation channel subfamily M member 4 | CTCCTTTGAGCCTTT |
| | (TRPM4), RefSeqGene on [symbol] | TGTACTCTCGCCTTC |
| SEQ ID | GI 1013417860 NG_046847 Homo sapiens | GGAGGTCATTCATGC |
| NO: 3219 | ubiquitin specific peptidase 7 | CCGTCAAGGTTGTTG |
| | (USP7), RefSeqGene on chromosome 16 | TGAGACTGGAATCGC |
| | | CAGGCACATCTAAAG |
| SEQ ID | GI 371875636 NM_182919 Homo sapiens | GCCTTCGACATTCTA |
| NO: 3220 | toll like receptor adaptor molecule 1 | GGTGCAGCAGGCCAG |
| | (TICAM1), mRNA | GACAAGCTCTTGTAT |
| | | CTGAAGCACAAACTG |
| SEQ ID | GI 256220780 NG_012741 Homo sapiens | GTCACGTACACTGCC |
| NO: 3221 | very low density lipoprotein receptor | AGGTAGCATAGGTTG |
| | (VLDLR), RefSeqGene on chromosome 9 | GCACAGCTTCTAAGG |
| | | AGTGGAGCTGATGAA |
| SEQ ID | GI 806607 M90696 Homo sapiens | TGCACTCATACGATC |
| NO: 3222 | cathepsin S | TGGGCATGAACCACC |
| | (CTSS) mRNA, complete cds | TGGGAGACATGACCA |
| | | GTGAAGAAGTGATGT |
| SEQ ID | GI 55953136 NM_002163 Homo sapiens | GGCCCGCATCATGAT |
| NO: 3223 | interferon regulatory factor 8 | TAAAGAATGTGGATC |
| | (IRF8), mRNA | CCTCTGTCTGGGGTG |
| | | GGATGCCTTACTTTG |
| SEQ ID | GI 166235119 NG_007149 Homo sapiens | GCTTTCTGGCTTCAC |
| NO: 3224 | B-cell CLL/lymphoma 6 | GAACAATTTCTCTCT |
| | (BCL6), RefSeqGene on chromosome 3 | CCTCGGCTCATGGGG |
| | | CAGTATGAAGGCATG |
| SEQ ID | GI 363807289 NG_031933 Homo sapiens | GATTTGGACATGTGG |
| NO: 3225 | BMP binding endothelial regulator | GCCCTGCACATTCAT |
| | (BMPER), RefSeqGene on chromosome 7 | ACAGCCTTCTGTGCT |
| | | TGGGACTAGAAGTCC |
| SEQ ID | GI 226492652 NG_011814 Homo sapiens | GAGCTGAGGATTTCT |
| NO: 3226 | C-X-C motif chemokine receptor 1 | GCTGCCCGCTTCCTC |
| | (CXCR1), RefSeqGene on chromosome 2 | TCTTGTGACCACCAC |
| | | TCATACATGGAGTTA |
| SEQ ID | GI 223718000 NG_009785 Homo sapiens | TCTTGCCTGGCCACT |
| NO: 3227 | guanidinoacetate N-methyltransferase | CTTGCAAATCCAACT |
| | (GAMT), RefSeqGene on chromosome 19 | TTCGGATAGAGGGAG |
| | | GGGGAGAGAAAAGTA |
| SEQ ID | GI 354725887 NG_030444 Homo sapiens | GTCAGGAACTCTAGC |
| NO: 3228 | hepatitis A virus cellular receptor 2 | AGTTGAGCCCAGCAA |
| | (HAVCR2), RefSeqGene on chromosome 5 | TCTGTGTTTTCGCAA |
| | | GGCTTCCCAGTGATT |
| SEQ ID | GI 237858687 NG_012088 Homo sapiens | CATTCTATCCATCCA |
| NO: 3229 | interleukin 10 | TAGGTGCCTGCCTTC |
| | (IL10), RefSeqGene on chromosome 1 | TCACTTCCCTCTCTT |
| | | CATGGGCTCTTGCCT |
| SEQ ID | GI 430768625 NG_033035 Homo sapiens | TGAATGGCACCACCA |
| NO: 3230 | kelch like family member 40 | TCCATTCAGTTGCTC |
| | (KLHL40), RefSeqGene on chromosome 3 | AGGGTATAAGCCTTG |
| | | GTGCCATCTTGGATT |
| SEQ ID | GI 574290111 NG_033893 Homo sapiens | GTCGTGATACAGCCC |
| NO: 3231 | MALTI paracaspase | AGATTCACAGGTGGG |
| | (MALT1), RefSeqGene on chromosome 18 | GGTGACTGTACTACA |
| | | CAAGGGCAAGGATAC |
| SEQ ID | GI 209870107 NG_008768 Homo sapiens | AGTGGAAGCAGCAGT |
| NO: 3232 | propionyl-CoA carboxylase alpha subunit | GGTGATCTGTAGTGG |
| | (PCCA), RefSeqGene on chromosome 13 | TGTCTACAGTGTGCT |
| | | GGTTCTGGTTGAGTT |
| SEQ ID | GI 215422398 NG_009053 Homo sapiens | AAGTCTGTTGTTTGC |
| NO: 3233 | protein activator of interferon | TGTCCCTACTCCTCA |
| | induced protein kinase EIF2AK2 | TGTGTTCTTGGCCAG |
| | (PRKRA), RefSeqGene on chro [symbol] | CTGTGGGTTTGGAAG |
| SEQ ID | GI 117606535 EF064742 Homo sapiens | GTTGGATCCGTGGAT |
| NO: 3234 | chemokine | ATGCTAGGAGCGGAG |
| | (C-X-C motif) ligand 16 | AAGAGGATTTTTCAG |
| | (CXCL16) gene, complete cds | GGCTGAGGATGGGAG |
| SEQ ID | GI 50234988 AY675320 Homo sapiens | TATTGGCTAACACCG |
| NO: 3235 | RAD21 homolog | CAACTCTCAAAGACC |
| | (S. pombe) | CTCGCAAGAGAATCA |
| | (RAD21) gene, complete cds | CCTTTGCGCTTGCTC |
| SEQ ID | GI 757813200 NG_012843 Homo sapiens | CTTCAGGGGALTGCT |
| NO: 3236 | lipin 1 | CCTATTTTTGTTGCT |
| | (LPIN1), RefSeqGene on chromosome 2 | CCTTAAAGCCTTACT |
| | | GTGGTTCCCCAGGGC |
| SEQ ID | GI 227908850 NG_011958 Homo sapiens | TCATGGTCCTTGAAA |
| NO: 3237 | noggin | GGTAGAGTGTCTCAC |
| | (NOG), RefSeqGene on chromosome 17 | CCAAGGTCGCACAGC |
| | | TGGTAGATTGCAAGT |
| SEQ ID | GI 1024846751 NG_047008 Homo sapiens | CGGGATCAAAGTGGG |
| NO: 3238 | pyruvate dehydrogenase kinase 4 | TCTACACCAGTGCTG |
| | (PDK4), RefSeqGene on chromosome 7 | CTTCCTGAATGTTTG |
| | | TGTGTGAACCCTTGT |
| SEQ ID | GI 340523097 NG_029467 Homo sapiens | TTCCCCACCCCAGAG |
| NO: 3239 | protein tyrosine kinase 2 | CACAAGGTTTAAAGA |
| | (PTK2), RefSeqGene on chromosome 8 | CTAGAACGTGGACTC |
| | | TTTTCACCCTCAGCT |
| SEQ ID | GI 37675281 NM_020664 Homo sapiens | AGGTCCGCCCCTCTG |
| NO: 3240 | 2,4-dienoyl-CoA reductase 2 | TCTCTGCACCACCTG |
| | (DECR2), mRNA | TTTGCATAAACACAC |
| | | TTTGCTACAATCTTG |
| SEQ ID | GI 194018480 NM_001945 Homo sapiens | CACACCAAACAAGGA |
| NO: 3241 | heparin binding EGF like growth factor | GGAGCACGGGAAAAG |
| | (HBEGF), mRNA | AAAGAAGAAAGGCAA |
| | | GGGGCTAGGGAAGAA |
| SEQ ID | GI 523425747 NM_004599 Homo sapiens | GTGTTGTGTGTATGT |
| NO: 3242 | sterol regulatory element | CCTGTGCCTTTTCTC |
| | binding transcription factor 2 | ACTATTGTTTGGGTG |
| | (SREBF2), transcript variant 1, | TGGGAGGGGGTGGTT |
| | mRN [symbol] | |
| SEQ ID | GI 40787172 AY513239 Homo sapiens | CCAGGCTCCATCCAT |
| NO: 3243 | complement component 3 | TGATAGGAGCCTTGC |
| | (C3) gene, complete cds | TCTATTGTTCTACAG |
| | | GGCCTTTGCACATGC |
| SEQ ID | GI 57790279 AY865620 Homo sapiens | CAGCATTTCCTCACC |
| NO: 3244 | signal transducer and activator | CGTTTCACATGAGCA |
| | of transcription 1, 91 kDa | GATTTGACACGTTGC |
| | (STAT1) gene, complete cds | TCTGTCCCCAGACTT |
| SEQ ID | GI 270289740 NG_015895 Homo sapiens | ATTCCCTGCCCTACA |
| NO: 3245 | four and a half LIM domains 1 | ACCCAGTGGGCCCTC |
| | (FHL1), RefSeqGene on chromosome X | TTTTGACCCTTGCAT |
| | | TTGTGGTATCATTAG |
| SEQ ID | GI 678246479 NG_034239 Homo sapiens | CTTCTCAGGATGCCA |
| NO: 3246 | lipopolysaccharide binding protein | CCGGGGGTCATTAGA |
| | (LBP), RefSeqGene on chromosome 20 | TTCATGGGTTCTGAT |
| | | GCAGACTGCAAAACC |
| SEQ ID | GI 1027250682 NG_047124 Homo sapiens | AGGGCCTTGCAAGGT |
| NO: 3247 | solute carrier family 27 member 5 | CCTGGTCATATCTAT |
| | (SLC27A5), RefSeqGene on chromosome 19 | TCTCACTGCCATTTG |
| | | TCTGTCCGTCTTCCA |
| SEQ ID | GI 292658763 NM_014143 Homo sapiens | GGCCCAAGCACTGAA |
| NO: 3248 | CD274 molecule | AATGGAACCTGGCGA |
| | (CD274), transcript variant 1, mRNA | AAGCAGAGGAGGAGA |
| | | ATGAAGAAAGATGGA |
| SEQ ID | GI 269914182 NM_178859 Homo sapiens | GCTGTGGTGGTCATT |
| NO: 3249 | solute carrier family 51 beta subunit | ATAAGCATGGTCCTC |
| | (SLC51B), mRNA | CTGGGAAGAAGCATC |
| | | CAGGCAAGCAGAAAA |
| SEQ ID | GI 343488502 NG_029727 Homo sapiens | GTCGTAGGGAGGGTT |
| NO: 3250 | platelet derived growth factor subunit A | GTAAACTGGTTGAGG |
| | (PDGFA), RefSeqGene on chromosome 7 | TCAGTTCTCATTGGG |
| | | TAAGCATCCCTCCGG |
| SEQ ID | GI 221316649 NM_005232 Homo sapiens | CTGGCTGCCAGAAAC |
| NO: 3251 | EPH receptor A1 | ATCTTGGTGAATCAA |
| | (EPHAl), mRNA | AACCTGTGCTGCAAG |
| | | GTGTCTGACTTTGGC |
| SEQ ID | GI 523498473 NM_006332 Homo sapiens | GAGGAGTGCAAATTC |
| NO: 3252 | IFI30, lysosomal thiol reductase | AACAAGGTGGAGGCC |
| | (IFI30), mRNA | TGCGTGTTGGATGAA |
| | | CTTGACATGGAGCTA |
| SEQ ID | GI 24430203 NM_153480 Homo sapiens | GTCCAGAAAATTGTG |
| NO: 3253 | interleukin 17 receptor E | TCTGGGGGCCACACT |
| | (IL17RE), transcript variant 1, mRNA | GTAGAGCTGCCTTAT |
| | | GAATTCCTTCTGCCC |
| SEQ ID | GI 53759150 NM_005063 Homo sapiens | TTGTGCGCAAACACC |
| NO: 3254 | stearoyl-CoA desaturase | CAGCTGTCAAAGAGA |
| | (SCD), mRNA | AGGGGAGTACGCTAG |
| | | ACTTGTCTGACCTAG |
| SEQ ID | GI 960513892 NM_001317781 Homo sapiens | GATTGGTGGTGTGAA |
| NO: 3255 | sorting nexin 25 | AACCTTGGCATGTGG |
| | (SNX25), mRNA | AAAGCCTCCATCACC |
| | | AGTGGAGAGGTTACA |
| SEQ ID | GI 8248053 AF199441 Homo sapiens | TTGGGAGGCTGTTAG |
| NO: 3256 | gastrointestinal glutathione peroxidase | GAGGATTACAGTCAG |
| | (GPX2) gene, complete cds | GAAGCATCAATAGCA |
| | | CTGTGGGTACTGGGC |
| SEQ ID | GI 238776813 NG_012250 Homo sapiens | ACCTACAGATCACAG |
| NO: 3257 | aldehyde dehydrogenase 2 family | GGAAGCAGGAAGATC |
| | (mitochondrial) | TAACGGCTTCTCTGT |
| | (ALDH2), RefSeqGene on chromosome 12 [symbol] | CCCCCTTACAGGTCA |
| SEQ ID | GI 193794846 NG_008174 Homo sapiens | ATCATTTGGGGATCT |
| NO: 3258 | fructose-bisphosphatase 1 | TGGTAAGATGTGTGC |
| | (FBP1), RefSeqGene on chromosome 9 | CCTCCTTCCATAGCT |
| | | CTGGGACAGGTCCTG |
| SEQ ID | GI 255958254 NG_012690 Homo sapiens | AATAAGAAATGCCAG |
| NO: 3259 | flavin containing monooxygenase 3 | CATCCACCAGAAGCC |
| | (FMO3), RefSeqGene on chromosome 1 | AGAAGCGGCAAGGAA |
| | | TGGATTCTCTCCTGG |
| SEQ ID | GI 345525392 NG_029921 Homo sapiens | TTCTGCCCCAGCTTT |
| NO: 3260 | major histocompatibility complex, | GCAGGATGAAAAGCT |
| | class II, DR beta 1 | TTCCCTCCTGGCTGT |
| | (HLA-DRB1), RefSeqGene on | TATTCTTCCACAAGA |
| | chromoso [symbol] | |
| SEQ ID | GI 343403846 NG_029699 Homo sapiens | CTCTGTACAGTGGGA |
| NO: 3261 | protein kinase cAMP-activated | ATAACAGCAGCTCCC |
| | catalytic subunit alpha | ACCCCACAGGGTGAG |
| | (PRKACA), RefSeqGene on chromoson | AACGAAGCATAGAAA |
| SEQ ID | GI 300244554 NG_023330 Homo sapiens | AGAGTGTTCACATGA |
| NO: 3262 | SMAD family member 7 | CTGAACCTAGGGGAG |
| | (SMAD7), RefSeqGene on chromosome 18 | GCCAGTTGTGACTTG |
| | | TGGCTGTCAGAAGTC |
| SEQ ID | GI 255652922 NG_012646 Homo sapiens | GTTGAGTGTTAGATG |
| NO: 3263 | transforming growth factor beta induced | CAGAGAAGAGACACC |
| | (TGFBI), RefSeqGene on chromosome 5 | CCCCACCTCCCCGCA |
| | | TCACTTACAGGAATT |
| SEQ ID | GI 209447082 NG_008731 Homo sapiens | ACAAAGAAGAGGGAG |
| NO: 3264 | vitamin D receptor | ATGACAAATGGGTAG |
| | (VDR), RefSeqGene on chromosome 12 | GGCTGGGGAAGGGGA |
| | | ATCATAGCTTGGTGA |
| SEQ ID | GI 222080101 NM_022121 Homo sapiens | GAGAGTGATTGGAGG |
| NO: 3265 | PERP, TP53 apoptosis effector | TCTCCTTGCCTTGGC |
| | (PERP), mRNA | TGCTGTGTTCCAGAT |
| | | CATCTCCCTGGTAAT |
| SEQ ID | GI 339515664 NM_003943 Homo sapiens | GCATCTTTAGATGGA |
| NO: 3266 | starch binding domain 1 | TGGTCCTCAGTCCAG |
| | (STBD1), mRNA | TCCTTCCATTTTACA |
| | | GGCGAGGAGGCGGAA |
| SEQ ID | GI 21435968 AF518727 Homo sapiens | CTTGCTCGCACAGCT |
| NO: 3267 | cyclin El | GTTGGCAGGAGCTGT |
| | (CCNEl) gene, complete cds | CATGGATAATTTGCA |
| | | TGCCCAATTCTTTGT |
| SEQ ID | GI 9837432 AF287892 Homo sapiens | GTCCTCTAGGACCGT |
| NO: 3268 | sialic acid binding immunoglobulin- | GTGTGATTTCACTGC |
| | like lectin 8 long splice variant | ACAGATGGACTCTGA |
| | (Siglec8) gene, complete cds | CTTTGTGGCATCCCT |
| SEQ ID | GI 255652911 NG_012637 Homo sapiens | TAAGAGTGCAGGCTT |
| NO: 3269 | C-C motif chemokine receptor 5 | CCCGCATTCAAAATC |
| | (gene/pseudogene) | GGTTGCTTACTAGCT |
| | (CCR5), RefSeqGene on chromosome 3 | GTGTGGCTTTGAGCA |
| SEQ ID | GI 239787264 NG_012303 Homo sapiens | CTCATCTTCCTCCTC |
| NO: 3270 | colony stimulating factor 1 receptor | TCAGTTCCCTTCTGG |
| | (CSF1R), RefSeqGene on chromosome 5 | TCCTTTCTGGCCAGG |
| | | TCTCTGTCTTCCTCT |
| SEQ ID | GI 299758395 NG_011675 Homo sapiens | ATGGGTGTTTGGAAG |
| NO: 3271 | glial cell derived neurotrophic factor | GGAAGGGAAAAGGAA |
| | (GDNF), RefSeqGene on chromosome 5 | AGGGGGACATACTAG |
| | | GGAGTAGCATGGGAA |
| SEQ ID | GI 296010831 NG_021194 Homo sapiens | TGCTTTGATCTTGAG |
| NO: 3272 | interleukin 1 receptor associated kinase 3 | GCAGCCCCCTGTCAA |
| | (IRAK3), RefSeqGene on chromosome 12 | CTTGTAGTGAATTGT |
| | | GCCCTGGTTCTTCAG |
| SEQ ID | GI 1007399066 NG_029521 Homo sapiens | AGGCCATTCCCTATG |
| NO: 3273 | SGT1 homolog, MIS12 kinetochore | CTCTGGAGACCTACC |
| | complex assembly cochaperone | CCTTCACTCTTGCAA |
| | (SUGT1), RefSeqGene < | ATGCTTCTACCCTTC |
| SEQ ID | GI 542133069 NM_004335 Homo sapiens | TGTAAGCTTCTGCTG |
| NO: 3274 | bone marrow stromal cell antigen 2 | GGGATAGGAATTCTG |
| | (BST2), mRNA | GTGCTCCTGATCATC |
| | | GTGATTCTGGGGGTG |
| SEQ ID | GI 514052655 NM_001278549 Homo sapiens | GAAAACAGTTGGCCT |
| NO: 3275 | pyruvate dehydrogenase kinase 1 | GTAATCACAGAACCA |
| | (PDK1), transcript variant 1, mRNA | GCCACCCTTCGGCGT |
| | | CTACATGCATTGTCT |
| SEQ ID | GI 328751683 NG_028290 Homo sapiens | TGGAGAAAAACTTCA |
| NO: 3276 | acyl-CoA synthetase long | TGATGCAGAAATGTG |
| | chain family member 6 | CTATTGCCTGGCTGG |
| | (ACSL6), RefSeqGene on chromosome 5 | GGAGGGCAGACATCA |
| SEQ ID | GI 269784608 NG_015894 Homo sapiens | GGAGTGAAAATGAGG |
| NO: 3277 | apolipoprotein A5 | GCTGTGTACTTGAGG |
| | (APOA5), RefSeqGene on chromosome 11 | CTGGGATTGGGGAAG |
| | | GTAGTGATGAGGACA |
| SEQ ID | GI 1064244211 NG_051562 Homo sapiens | TTCTTGTCCCTCTGT |
| NO: 3278 | ChaC glutathione specific gamma- | CTGTCTGTCTATCTA |
| | glutamylcyclotransferase 1 | CTTGTCTGTCTGGGC |
| | (CHAC1), RefSeqGene on chro [symbol] | CACTCCTGCCTGTGT |
| SEQ ID | GI 225637493 NG_011688 Homo sapiens | ATGCACATTACAGGC |
| NO: 3279 | growth hormone receptor | ATGGTAAAAAGAGGC |
| | (GHR), RefSeqGene on chromosome 5 | TCTGGGAAGCATGTT |
| | | CGAGCTGCTCTGCTC |
| SEQ ID | GI 818213488 NG_042142 Homo sapiens | TTCCAGCATACGGCA |
| NO: 3280 | inositol 1,4,5-trisphosphate | TTCCTTTTCTCTCTT |
| | receptor type 2 | CACACCATGCTTCTC |
| | (ITPR2), RefSeqGene on chromosome 12 | AGAAAGGCCTTGGCC |
| SEQ ID | GI 308199420 NG_027788 Homo sapiens | TGTAAAGTAGCGTGC |
| NO: 3281 | MX dynamin like GTPase 1 | ATACAGGGATGGAAG |
| | (MX1), RefSeqGene on chromosome 21 | GGAATGTGGATGCCA |
| | | TTGTGCCAGCAAGTT |
| SEQ ID | GI 194394200 NG_008275 Homo sapiens | CCCTCCTAGCTGAGT |
| NO: 3282 | prokineticin 2 | AACCTCAGTCCAGGA |
| | (PROK2), RefSeqGene on chromosome 3 | CCTTTGCTTCACCTC |
| | | AAAAGGAAGCCACTT |
| SEQ ID | GI 346644679 NG_029975 Homo sapiens | GGCCACTTTCTCTTA |
| NO: 3283 | transforming growth factor alpha | TCCTACCCACCAGCC |
| | (TGFA), RefSeqGene on chromosome 2 | TGGCCCTCCTTTCTA |
| | | CAGCTGGTTCTTAAT |
| SEQ ID | GI 224809368 NG_011523 Homo sapiens | AGCAACTCTGAGCAG |
| NO: 3284 | TIR domain containing adaptor protein | TAGCCTCTTCCCCAT |
| | (TIRAP), RefSeqGene on chromosome 11 | TTAGCGACAATCTAG |
| | | GATTTCTTGGGGCCA |
| SEQ ID | GI 671744853 NG_034216 Homo sapiens | TGGTGATCAGTCAAC |
| NO: 3285 | vascular endothelial growth factor C | CCAGCCTTACTTTCC |
| | (VEGFC), RefSeqGene on chromosome 4 | TGGAGTGTTCATAGT |
| | | CTGCAGAGCACCAGG |
| SEQ ID | GI 81230486 NM_001037162 Homo sapiens | CTGTGCACGCTGTTT |
| NO: 3286 | acyl-CoA thioesterase 6 | TGGGTGAGGCAATAT |
| | (ACOT6), mRNA | TCTATGGAGGTGAGC |
| | | CAAAGGCTCACTCAA |
| SEQ ID | GI 169790843 NM_000239 Homo sapiens | GGCGTGAGCCACTGC |
| NO: 3287 | lysozyme | GCCCGGCCACATTCA |
| | (LYZ), mRNA | GTTCTTATCAAAGAA |
| | | ATAACCCAGACTTAA |
| SEQ ID | GI 1147566 X80590 H. sapiens | GGAAACCAGAAAGAT |
| NO: 3288 | PHKGI mRNA | CATGCGAGCTCTGCT |
| | | GGAGGTGATCTGCAC |
| | | CTTGCACAAACTCAA |
| SEQ ID | GI 1015224512 NG_023402 Homo sapiens | GCAAGCACTGCCTAT |
| NO: 3289 | Janus kinase 1 | GGACTGGCTAGTGCT |
| | (JAK1), RefSeqGene on chromosome 1 | GTTGGGTGCCCAAGT |
| | | GTAAAAAGGAGTATT |
| SEQ ID | GI 237858777 NG_012128 Homo sapiens | TAGGATTGCACAGTG |
| NO: 3290 | solute carrier family 11 member 1 | AGTGACAAGCCATGA |
| | (SLC11A1), RefSeqGene on chromosome 2 | TGCTGCTGTCTCCTC |
| | | TCTGGACCCATCATT |
| SEQ ID | GI 323098322 NM_005624 Homo sapiens | GAAATAAGGTTTTTG |
| NO: 3291 | C-C motif chemokine ligand 25 | CAAAGCTCCACCACA |
| | (CCL25), transcript variant 1, mRNA | ACACGCAGACCTTCC |
| | | AAGCAGGCCCTCATG |
| SEQ ID | GI 117606541 EF064745 Homo sapiens | GTCTACACTGTGCTG |
| NO: 3292 | chemokine | AGTCTGAAGGCTGGG |
| | (C-X3-C motif) ligand 1 | TTCTGATCCTAGTTC |
| | (CX3CL1) gene, complete cds | CACCATCAAGCCACC |
| SEQ ID | GI 294489317 NG_017064 Homo sapiens | AGGAGTCGAGGAGGA |
| NO: 3293 | acyl-CoA dehydrogenase family member 9 | GAGGAAGCCATCACG |
| | (ACAD9), RefSeqGene on chromosome 3 | TTCACCTGACCTAGT |
| | | TTTCTGGGAGAAGTT |
| SEQ ID | GI 225543278 NG_011648 Homo sapiens | GCCCTGAATGTGTGA |
| NO: 3294 | angiotensin 1 converting enzyme | AGTAGGCGGTGAGGA |
| | (ACE), RefSeqGene on chromosome 17 | CAGTAAGGGATGGTA |
| | | GGGAGTAAGGCAAAG |
| SEQ ID | GI 334085262 NG_029008 Homo sapiens | GAAATAGTGGTAGCA |
| NO: 3295 | CRK proto-oncogene, adaptor protein | GAAAGCCTTCAGTGA |
| | (CRK), RefSeqGene on chromosome 17 | CCAGCCCAACCCAGT |
| | | TTAAGGCGATGCCTG |
| SEQ ID | GI 582015216 NG_016002 Homo sapiens | ATGGGCCTGTGTGGA |
| NO: 3296 | G protein subunit alpha i2 | CAGCTTCACAAAAAA |
| | (GNAI2), RefSeqGene on chromosome 3 | AGGAGACAGTGTCCA |
| | | CCATTGGCTCCATAA |
| SEQ ID | GI 291045455 NG_016708 Homo sapiens | CTCAGAGGATTGGTT |
| NO: 3297 | MDM2 proto-oncogene | TGACCAGGCATGTCA |
| | (MDM2), RefSeqGene on chromosome 12 | TTTAGCCCTCAAGAA |
| | | AACTGGCCCTCCCAC |
| SEQ ID | GI 116006947 NM_001285 Homo sapiens | ACTGAGCAGATGGGC |
| NO: 3298 | chloride channel accessory 1 | AACTGTGGAGAGAAG |
| | (CLCA1), mRNA | GGTGAAAGGATCCAC |
| | | CTCACTCCTGATTTC |
| SEQ ID | GI 399567818 NM_003299 Homo sapiens | TTGTCAAGGGTGTGG |
| NO: 3299 | heat shock protein 90 beta | TGGACTCAGATGATC |
| | family member 1 | TCCCCTTGAATGTTT |
| | (HSP90B1), mRNA | CCCGCGAGACTCTTC |
| SEQ ID | GI 188219598 NM_004688 Homo sapiens | TATGACAGACAGTCC |
| NO: 3300 | N-myc and STAT interactor | GGGAGTGCAGTCATC |
| | (NMI), mRNA | ACGTTTGTGGAGATT |
| | | GGAGTGGCTGACAAG |
| SEQ ID | GI 117606569 EF064759 Homo sapiens | CAACCTCAGCACCAT |
| NO: 3301 | chemokine | TGATCTTCTTGGTCA |
| | (C-C motif) receptor 4 | GACAACTCTTTGCTG |
| | (CCR4) gene, complete cds | TGGTGGGCTGTCCTG |
| SEQ ID | GI 224809308 NG_011502 Homo sapiens | CAGAAGCCACCCTGA |
| NO: 3302 | cytotoxic T-lymphocyte | CAGGGAACTAATGAG |
| | associated protein 4 | ACATTTTCTACAGGG |
| | (CTLA4), RefSeqGene on chromosome 2 | GTCTGTCCCAGGTGG |
| SEQ ID | GI 343098522 NG_029664 Homo sapiens | CCAAAGGCCAAGGTC |
| NO: 3303 | cathepsin E | CAAACTGAACTCAGT |
| | (CTSE), RefSeqGene on chromosome 1 | GCTGAGCTCATGAGC |
| | | TGTTCCTCAAACTCC |
| SEQ ID | GI 393533306 NG_032816 Homo sapiens | CATTCTGCTGTACCA |
| NO: 3304 | 5-hydroxytryptamine receptor 1A | AGGGTCTGCGGAAAC |
| | (HTRIA), RefSeqGene on chromosome 5 | CACCTTTTTGCCCTT |
| | | TGAATTGTGCAGCAT |
| SEQ ID | GI 1016841222 NG_008723 Homo sapiens | TGTAGCCCATTTTCC |
| NO: 3305 | monoamine oxidase B | TGTGAAAGGCCTTGA |
| | (MAOB), RefSeqGene on chromosome X | CTTGGATTGAAGCTG |
| | | GGCTCCTCCAACTTC |
| SEQ ID | GI 340007370 NG_029431 Homo sapiens | GTTCTCAGATGCGTA |
| NO: 3306 | ras-related C3 botulinum | AAGCAGAACAGCCTC |
| | toxin substrate 1 | CCGAATGAAGCGTTG |
| | (rho family, small GTP | CCATTGAACTCACCA |
| | binding protein Rael) | |
| | (RAC [symbol] | |
| SEQ ID | GI 959241467 NG_046314 Homo sapiens | ACTGGGGTAGCTGGC |
| NO: 3307 | signal transducer and | CTACCAGGTAGCTCA |
| | activator of transcription 2 | GATGGTGATTGAGTT |
| | (STAT2), RefSeqGene on chromosome 12 | TGCTGAGAAACCATC |
| SEQ ID | GI 1024336756 NG_046908 Homo sapiens | AGTGGGAGATTGACT |
| NO: 3308 | YYl transcription factor | GCCAAAGAGGAAGTA |
| | (YYl), RefSeqGene on chromosome 14 | GGAGAGGTGTGAGAG |
| | | GTTGGAGTCCAGTGA |
| SEQ ID | GI 748585173 NM_198580 Homo sapiens | CTTCTGGGACGACTG |
| NO: 3309 | solute carrier family 27 member 1 | CATCAAGTACAACTG |
| | (SLC27A1), mRNA | CACGGTGGTTCAGTA |
| | | CATCGGGGAGATCTG |
| SEQ ID | GI 1049010711 AH003036 Homo sapiens | AAGGAGCTGGGGACT |
| NO: 3310 | Bruton agammaglobulinemia | GGACAATTTGGGGTA |
| | (BTK) gene, complete cds | GTGAAGTATGGGAAA |
| | | TGGAGAGGCCAGTAC |
| SEQ ID | GI 56788327 AY851164 Homo sapiens | ATCTGGGGTTAATGA |
| NO: 3311 | carboxylesterase 2 | ACCCAGGCTTTGATA |
| | (intestine, liver) | CAGGGGGTTCGATTG |
| | (CES2) gene, complete cds | CAAAGATTGGGAGGC |
| SEQ ID | GI 14488405 AF386077 Homo sapiens | AGATTTCAGGAGGGA |
| NO: 3312 | interleukin 17B | ATGGGCCAGGCCACC |
| | (IL17B) gene, complete cds | GTCTACTTCTGGATA |
| | | CACCCACCATTTTAT |
| SEQ ID | GI 269308199 NG_015892 Homo sapiens | CTCCAGCTTTCCCCC |
| NO: 3313 | bassoon presynaptic cytomatrix protein | AGGGATTTGCAGCCT |
| | (BSN), RefSeqGene on chromosome 3 | CATTTTCCCCATCCA |
| | | TAGTCCATTTTGCTA |
| SEQ ID | GI 1024846763 NG_046978 Homo sapiens | TGGCAGACAATGAGA |
| NO: 3314 | Charcot-Leyden crystal galectin | AGTGGCCATGCTATG |
| | (CLC), RefSeqGene on chromosome 19 | GCTATTCTGTGAGAG |
| | | TAGAGGCATGGGGTT |
| SEQ ID | GI 334085262 NG_029008 Homo sapiens | CTTCCTTTCCCGCCC |
| NO: 3315 | CRK proto-oncogene, adaptor protein | TTCTATTTCCTGCCT |
| | (CRK), RefSeqGene on chromosome 17 | TTGGCTGTTTGTGGT |
| | | TGGAAGGAGAGGAAA |
| SEQ ID | GI 1024846760 NG_046988 Homo sapiens | TGAGCTATGAATGCC |
| NO: 3316 | E2F transcription factor 1 | CCTCTCTGAGCCTCT |
| | (E2F1), RefSeqGene on chromosome 20 | TCCTTCCTGCACCAT |
| | | GTGGATAAACAAGCT |
| SEQ ID | GI 339515631 NG_029366 Homo sapiens | AGCTCCCGCTTTAAC |
| NO: 3317 | coagulation factor III, tissue factor | TCTGGGGAAAGGGCT |
| | (F3), RefSeqGene on chromosome 1 | TCCCAGTGAGTTGCG |
| | | ACCTTCAATATGATA |
| SEQ ID | GI 291084780 NG_016747 Homo sapiens | CGCAGAACCTCTTTC |
| NO: 3318 | PPARG coactivator 1 beta | CTCATCTGCAAGGTG |
| | (PPARGC1B), RefSeqGene on chromosome 5 | GAGATAACAACAGTA |
| | | CCCACCTCACAGGCT |
| SEQ ID | GI 238018099 NG_012213 Homo sapiens | AGCCAGGTGACTGCA |
| NO: 3319 | superoxide dismutase 3 | TCTTGCCTAGGCACA |
| | (SOD3), RefSeqGene on chromosome 4 | GAAGAGTAGGGTTGG |
| | | AATGTGACGTTGTTA |
| SEQ ID | GI 574957093 NM_001252 Homo sapiens | CTGGACAAGGGGCAG |
| NO: 3320 | CD70 molecule | CTACGTATCCATCGT |
| | (CD70), transcript variant 1, mRNA | GATGGCATCTACATG |
| | | GTACACATCCAGGTG |
| SEQ ID | GI 164695749 AK315559 Homo sapiens | GCTGGAAAAACAGTG |
| NO: 3321 | cDNA, FLI96630, Homo sapiens | CCGATTGTGAAAGGA |
| | methionyl aminopeptidase 2 | GGGGAGGCAACAAGA |
| | (METAP2), mRNA | ATGGAGGAAGGAGAA |
| SEQ ID | GI 209571510 NM_015000 Homo sapiens | AACTGCACTGCCTAC |
| NO: 3322 | serine/threonine kinase 38 like | ATGCGTATTAAGGTC |
| | (STK38L), mRNA | CATTCTGCCTGTGTG |
| | | TGCTGTGGCTTTGAA |
| SEQ ID | GI 22128580 AF533019 Homo sapiens | TCAGTTTCCCCTTTT |
| NO: 3323 | growth arrest and DNA-damage- | GCATAATGCACATAA |
| | inducible, beta | GCATCCCCCCTGCTT |
| | (GADD45B) gene, complete cds | CCCAGGTCTTGGATT |
| SEQ ID | GI 14488404 AF385628 Homo sapiens | TTTCAGAGACAGCAG |
| NO: 3324 | interleukin 8 | AGCACACAAGCTTCT |
| | (IL8) gene, complete cds | AGGACAAGAGCCAGG |
| | | AAGAAACCACCGGAA |
| SEQ ID | GI 77744394 DQ230990 Homo sapiens | CATCTGGCAAGAAAT |
| NO: 3325 | peroxiredoxin 6 | ACCTCCGCTACACAC |
| | (PRDX6) gene, complete cds | CCCAGCCTTAAGTCT |
| | | CTTGGAGAAGCTGGT |
| SEQ ID | GI 224809296 NG_011498 Homo sapiens | TAGACCAGGCTGCCA |
| NO: 3326 | interleukin 23 receptor | GGAAAAGAAAAGAGC |
| | (IL23R), RefSeqGene on chromosome 1 | AGAGAGAAGTCATTG |
| | | GTGACATCCCACCCA |
| SEQ ID | GI 315360651 NG_028027 Homo sapiens | CAAGAGCCCAAGAGC |
| NO: 3327 | prodynorphin | CCCATGATACTGTTG |
| | (PDYN), RefSeqGene on chromosome 20 | TCCCCACTGTACAGA |
| | | TGAGCAATCAGAGGT |
| SEQ ID | GI 237858755 NG_012119 Homo sapiens | TTTTGAGTATCCAGG |
| NO: 3328 | protein tyrosine phosphatase, | AGGGCTGGGGACTTT |
| | non-receptor type 1 | GGAGTAGTTGGTGAT |
| | (PTPN1), RefSeqGene on | TCCCTCTGCCACATT |
| | chromosome 2 [symbol] | |
| SEQ ID | GI 187171274 NM_030662 Homo sapiens | CGGTGTGTTCACCCC |
| NO: 3329 | mitogen-activated | CGACTTCCAGGAGTT |
| | protein kinase kinase 2 | TGTCAATAAATGCCT |
| | (MAP2K2), mRNA | CATCAAGAACCCAGC |
| SEQ ID | GI 115387093 NM_003000 Homo sapiens | TGTGGCTCTTGTGCA |
| NO: 3330 | succinate dehydrogenase complex iron | ATGAACATCAATGGA |
| | sulfur subunit B | GGCAACACTCTAGCT |
| | (SDHB), Mrna | TGCACCCGAAGGATT |
| SEQ ID | GI 928192547 NM_006472 Homo sapiens | TGCCCAACCCTGTGA |
| NO: 3331 | thioredoxin interacting protein | TCAGAACCTCCAAAT |
| | (TXNIP), transcript variant 1, mRNA | ACTGCCATGAGAAAC |
| | | TAGAGGGCAGGTCTT |
| SEQ ID | GI 1212624021 NM_017523 Homo sapiens | TTGCCTGCAAGAAAC |
| NO: 3332 | XIAP associated factor 1 | GAAACTCAACCGAAA |
| | (XAF1), transcript variant 1, mRNA | GCCTGCAGAGAGCAG |
| | | AACATGGAAGGAGAC |
| SEQ ID | GI 24079970 AF550128 Homo sapiens | AGCAGCATGTGAGAA |
| NO: 3333 | excision repair cross-complementing | GAACCTGGAGGCAGG |
| | rodent repair deficiency, | AAGGAGCTTAGTGAT |
| | complementation group 5 | TTCTGGGTTTGAGGA |
| | (ERCC [symbol] | |
| SEQ ID | GI 27501935 AY207429 Homo sapiens | CCGTCTCCCTGTCTC |
| NO: 3334 | interleukin 11 | TGTCTCAGTCTGTCC |
| | (ILH) gene, complete cds | TTCACTCTGTGTGTG |
| | | TGTGTGTGTGTCTCT |
| SEQ ID | GI 937628170 KP290484 Homo sapiens | ACTTCTTCCTGTGCA |
| NO: 3335 | OR10J3 | TGCTATCCATCTCTG |
| | (OR10J3) gene, complete cds | AGACCTGCTACACTG |
| | | TGGCCATCATTCCCC |
| SEQ ID | GI 83699688 DQ323999 Homo sapiens | CATCTCTTAACTCAG |
| NO: 3336 | TNF receptor-associated factor 4 | CACCTCTGACATTTC |
| | (TRAF4) gene, complete cds | CAGAGCCATGAGGGT |
| | | ATGTGCCCCCAGGAG |
| SEQ ID | GI 219689119 NG_009261 Homo sapiens | CATCAGACTATTCCC |
| NO: 3337 | elastin | AGGAGGGGGTAGGAA |
| | (ELN), RefSeqGene on chromosome 7 | TCCTGGGTTTGAGTC |
| | | CCAGCTCAGACACTA |
| SEQ ID | GI 1121393451 NG_012066 Homo sapiens | GGTTCAGCATGGGCA |
| NO: 3338 | Fc fragment of IgG receptor Ila | GTTCCCCCATTTTAG |
| | (FCGR2A), RefSeqGene on chromosome 1 | TGGGGTCTGGGATTT |
| | | GCTTCTTAGATCCAC |
| SEQ ID | GI 334848111 NG_029036 Homo sapiens | GCTTGCTCATTCTAG |
| NO: 3339 | promyelocytic leukemia | AGGCAGCTGCTCCCA |
| | (PML), RefSeqGene on chromosome 15 | ACTCTGGACTGCTCT |
| | | GACTTAGAAAGTGCT |
| SEQ ID | GI 343432618 NG_029713 Homo sapiens | TTGGCCCTTGAGTCC |
| NO: 3340 | synoviolin 1 | AGTCACCCTGTCTGA |
| | (SYVN1), RefSeqGene on chromosome 11 | GTTCATTCAGCATGT |
| | | TCACTTGTGTTTGCC |
| SEQ ID | GI 8248053 AF199441 Homo sapiens | TCAGTGGCTTGGTGG |
| NO: 3341 | gastrointestinal glutathione peroxidase | GGACTCTTTCTTATT |
| | (GPX2) gene, complete cds | GGTTGCTTCTAGGAA |
| | | CTCCACCCAGCTTCA |
| SEQ ID | GI 306816 M96233 Human glutathione | GAGTCTTCTGCCCAA |
| NO: 3342 | transferase class mu number 4 | TTCCTCTCACTCCTG |
| | (GSTM4) gene, complete cds | GTTGTCTACACAGCC |
| | | CCTGCATGATGTTCT |
| SEQ ID | GI 14488404 AF385628 Homo sapiens | TAAATCTGGCAACCC |
| NO: 3343 | interleukin 8 | TAGTCTGCTAGCCAG |
| | (IL8) gene, complete cds | GATCCACAAGTCCTT |
| | | GTTCCACTGTGCCTT |
| SEQ ID | GI 334085206 NG_029005 Homo sapiens | GCTCCACTTCTATGG |
| NO: 3344 | CASP8 and FADD like apoptosis regulator | TGGTCACTCACATGA |
| | (CFLAR), RefSeqGene on chromosome 2 | CTGAGAAGTTGGTGC |
| | | TAGCTGTTGGCAGGA |
| SEQ ID | GI 742670592 NG_013347 Homo sapiens | CTGTGCCCTTGCCCC |
| NO: 3345 | glutamate-ammonia ligase | TTGCTCTTATCTTGG |
| | (GLUL), RefSeqGene on chromosome 1 | TCCGTGTATCTGGGA |
| | | GTTCTTCCACCTTAT |
| SEQ ID | GI 342307070 NG_029606 Homo sapiens | GTTCCGCAAGCCCTG |
| NO: 3346 | hypoxia inducible factor 1 alpha subunit | AAAGCGCAAGTCCTC |
| | (HIF1A), RefSeqGene on chromosome 14 | AAAGCACAGTTACAG |
| | | TATTCCAGCAGACTC |
| SEQ ID | GI 1024336778 NG_046901 Homo sapiens | CTAACCCGGCCATTG |
| NO: 3347 | NLR family pyrin domain containing 9 | GAAGCATAACCCAGC |
| | (NLRP9), RefSeqGene on chromosome 19 | TTGTAAGAGCAAGTG |
| | | TGGTTCAGCCTCAAA |
| SEQ ID | GI 1041818081 NG_051000 Homo sapiens | CAGGTCACATTGTTC |
| NO: 3348 | pentraxin 3 | CTGAGGGAGGAATCC |
| | (PTX3), RefSeqGene on chromosome 3 | TGCAGATTGGCCAAG |
| | | AAAAGAATGGCTGCT |
| SEQ ID | GI 364023822 NM_130898 Homo sapiens | AGGCCTTATCTCCAT |
| NO: 3349 | cAMP responsive element | CCAGCTAGATCAGTG |
| | binding protein 3 like 4 | GAGCCCAGCATTTAT |
| | (CREB3L4), transcript variant 1, mRNA | GGTGCCTGATTCCTG |
| SEQ ID | GI 735661405 NM_002201 Homo sapiens | CCCTATTCCTGGTGC |
| NO: 3350 | interferon stimulated exonuclease gene 20 | ATTTATGCCCAGAGA |
| | (ISG20), transcript variant 1, mRNA | GGTGGCATTATTTCC |
| | | TGGGGTGGCATTCAG |
| SEQ ID | GI 61744461 NM_003405 Homo sapiens | AGATCGAAATCTCCT |
| NO: 3351 | tyrosine 3-monooxygenase/tryptophan 5- | CTCTGTGGCCTACAA |
| | monooxygenase activation protein eta | GAATGTGGTTGGTGC |
| | (YWHAH), [symbol] | CAGGCGATCTTCCTG |
| SEQ ID | GI 117606529 EF064739 Homo sapiens | CACAGAACTCCCGTT |
| NO: 3352 | Fas ligand | GTGTGGCAATGCTCA |
| | (TNF superfamily, member 6) | GTGGGAGTAAGGAGA |
| | (FASLG) gene, complete cds | TAAGGCTGGACAGAT |
| SEQ ID | GI 834400732 NG_042181 Homo sapiens | GCCACCAGCCTCCAT |
| NO: 3353 | collagen type XII alpha 1 chain | CTGGATTCCCCTTTC |
| | (COL12A1), RefSeqGene on chromosome 6 | TTTTACTGGGGACTG |
| | | GAAATTCCAAGCAAT |
| SEQ ID | GI 1154067877 NG_052975 Homo sapiens | ATTCTATGCTGGGTA |
| NO: 3354 | C-X-C motif chemokine receptor 2 | CTTGCTAAACTCGGG |
| | (CXCR2), RefSeqGene on chromosome 2 | ACCAAGCACGACTCA |
| | | ACTGCTCTGGCTGAG |
| SEQ ID | GI 189217849 NG_007932 Homo sapiens | GGCCACCCCATGCAA |
| NO: 3355 | cytochrome P450 family | ATGATTGGTCTTCTC |
| | 4 subfamily A member 11 | TCTGTTTCCAACCTG |
| | (CYP4A11), RefSeqGene on chromosome [symbol] | CACCACAGGTATCAT |
| SEQ ID | GI 189458878 NM_139166 Homo sapiens | GAAATATAGCCCAGT |
| NO: 3356 | actin binding Rho activating protein | GGGCAACTTGAAAGG |
| | (ABRA), mRNA | GAGATGGCAGCAGTG |
| | | GGCTGATGAACACAT |
| SEQ ID | GI 325652058 NM_005524 Homo sapiens | AACTCCCCAACCCAC |
| NO: 3357 | hes family bHLH transcription factor 1 | CTCTCTTCCCTCCGG |
| | (HES1), mRNA | ACTCTAAACAGGAAC |
| | | TTGAATACTGGGAGA |
| SEQ ID | GI 20379137 AF498982 Homo sapiens | CGACTCTGAAAATCG |
| NO: 3358 | 5-hydroxytryptamine receptor 2A | AACCAACCTTTCCTG |
| | (HTR2A) mRNA, complete cds | TGAAGGGTGCCTCTC |
| | | ACCGTCGTGTCTCTC |
| SEQ ID | GI 45934429 AY585238 Homo sapiens | AGTACCCCGACCTCA |
| NO: 3359 | creatine kinase, muscle | GCAAACATAACAACC |
| | (CKM) gene, complete cds | ACATGGCCAAGGTAC |
| | | TGACCCTTGAACTCT |
| SEQ ID | GI 57790271 AY878121 Homo sapiens | CTCCCCTTTACTCTC |
| NO: 3360 | glutathione S-transferase A4 | TGAGTCTCACTCTCG |
| | (GSTA4) gene, complete cds | CATTCTTCCCTCACT |
| | | CCCCCAGTTCAGTCC |
| SEQ ID | GI 254911080 NG_012514 Homo sapiens | AATGGATTTGGAGAG |
| NO: 3361 | interleukin 13 receptor subunit alpha 2 | AGGGGTCAACTCTCA |
| | (IL13RA2), RefSeqGene on chromosome X | CTCCCCCAGTCCACT |
| | | GTTTTGGACACAACA |
| SEQ ID | GI 169790799 NG_007447 Homo sapiens | GCTCTTGGACTCCAG |
| NO: 3362 | TGFB induced factor homeobox 1 | ACACTGACTTCAACT |
| | (TGIF1), RefSeqGene on chromosome 18 | TACCACTTTCCGCCC |
| | | CGATTAGAAAGGCCT |
| SEQ ID | GI 554506569 NM_001114 Homo sapiens | GTTCCCTGAGGTGCC |
| NO: 3363 | adenylate cyclase 7 | AGGCAGGCAACTTTA |
| | (ADCY7), transcript variant 1, mRNA | GCACATGATGAAAAC |
| | | AGACTTCCACCTCAG |
| SEQ ID | GI 206597534 NM_001639 Homo sapiens | CTCATCCTGGTCACT |
| NO: 3364 | amyloid P component, serum | GCTTCTGCTATAACA |
| | (APCS), mRNA | GCCCTAGGCCAGGAA |
| | | TATGAACAAGCCGCT |
| SEQ ID | GI 167555126 NM_005582 Homo sapiens | CTCCAAGCATTAGGT |
| NO: 3365 | CD180 molecule | AAACCCACCAAGCAA |
| | (CD180), mRNA | TCCTAGCCTGTGATG |
| | | GCGTTTGACGTCAGC |
| SEQ ID | GI 735367774 NM_000211 Homo sapiens | GTGGAACAATGATAA |
| NO: 3366 | integrin subunit beta 2 | TCCCCTTTTCAAGAG |
| | (ITGB2), transcript variant 1, mRNA | CGCCACCACGACGGT |
| | | CATGAACCCCAAGTT |
| SEQ ID | GI 385137136 NM_012340 Homo sapiens | CTCACCCCTCTGGCC |
| NO: 3367 | nuclear factor of activated T-cells 2 | TCTACAGCCCTCAGA |
| | (NFATC2), transcript variant 1, mRNA | GGAGAGAAAACCAAT |
| | | TCTCCAACAAACAGG |
| SEQ ID | GI 214832245 NM_017853 Homo sapiens | ACTCCAGGAATAAGA |
| NO: 3368 | thioredoxin like 4B | CAAAAGGAGAGGAGG |
| | (TXNL4B), transcript variant 1, mRNA | AGGCCATCACCTCTG |
| | | GCCTCTTCCATCGTA |
| SEQ ID | GI 751368105 NM_145911 Homo sapiens | GTGTCATACCATCAC |
| NO: 3369 | zinc finger protein 23 | TGGAGAGCAGCCCTC |
| | (ZNF23), transcript variant 1, mRNA | TGGGTGTACAGGATT |
| | | GGGGAAATCCATCAG |
| SEQ ID | GI 261599023 NG_013217 Homo sapiens | TTGAATGCCCAGTGA |
| NO: 3370 | apoptosis inducing factor | AACAGAAACGGTGCA |
| | mitochondria associated 1 | GGGCAAGTAAGGAGT |
| | (AIFM1), RefSeqGene on chromosome [symbol] | CGGTTGGTTGTGTAC |
| SEQ ID | GI 307133697 NG_027730 Homo sapiens | GGTATGTTGTTCACT |
| NO: 3371 | C-C motif chemokine ligand 3 | CTCCAGCTCACTCTA |
| | (CCL3), RefSeqGene on chromosome 17 | GGTCTCCCAGGAGCT |
| | | CTGTCCCTTGGATGT |
| SEQ ID | GI 1002095861 NG_046750 Homo sapiens | GCCAGCCTTCTCTGG |
| NO: 3372 | cytochrome c oxidase subunit 8A | TCTTCCTTTTTGGTG |
| | (COX8A), RefSeqGene on chromosome 11 | GACTTCATTTCTTAG |
| | | GTGCTGGTGCCCTTC |
| SEQ ID | GI 197116369 NG_008383 Homo sapiens | TAACTATGGGTTGGC |
| NO: 3373 | cytochrome P450 family | AACATGTTCCTGGAT |
| | 2 subfamily E member 1 | GTGTTTGCTGGCACA |
| | (CYP2E1), RefSeqGene on | GTGACAGGTGCTAGC |
| | chromosome 10 [symbol] | |
| SEQ ID | GI 937500889 NG_032896 Homo sapiens | CTCCTTCCAAGCCAA |
| NO: 3374 | purinergic receptor P2Y1 | AAGAAGCCTCTGGGA |
| | (P2RY1), RefSeqGene on chromosome 3 | AGGTGTGCCAATGGT |
| | | GAAAACGTACAGAGG |
| SEQ ID | GI 1024846788 NG_047009 Homo sapiens | GGGCCATGAAGAATG |
| NO: 3375 | platelet and endothelial | AATGGAGTGATGGCA |
| | cell adhesion molecule 1 | GCATTGAGGGAGAAA |
| | (PECAM1), RefSeqGene on chromosome | GATCAGCAGGAGCAC |
| SEQ ID | GI 307611978 NM_005409 Homo sapiens | TTCAAGGCTTCCCCA |
| NO: 3376 | C-X-C motif chemokine ligand 11 | TGTTCAAAAGAGGAC |
| | (CXCL11), transcript variant 1, mRNA | GCTGTCTTTGCATAG |
| | | GCCCTGGGGTAAAAG |
| SEQ ID | GI 167234418 NM_005119 Homo sapiens | CCTATTTCCTGATTT |
| NO: 3377 | thyroid hormone receptor associated | CTTCTGGGCTGGACT |
| | protein 3 | TCCCCGTTCTCCACC |
| | (THRAP3), mRNA | AGCAGCTCCAGTATC |
| SEQ ID | GI 326535636 GU997628 Homo sapiens | GTGATAATACAGAAA |
| NO: 3378 | annexin A1 | GGTGGGGCTGGTGTA |
| | (ANXAl) gene, complete cds | GGGGTGAAGTCAGGA |
| | | TGCTTTGGGAGAGCA |
| SEQ ID | GI 68248543 DQ096732 Homo sapiens | CGAGTGCTAGCTGAC |
| NO: 3379 | glutathione peroxidase 7 | TGAGCCAAAAATTGA |
| | (GPX7) gene, complete cds | GCCCAGGTCTCCCTT |
| | | TGTACCCCCTCTTAT |
| SEQ ID | GI 974987497 NG_011800 Homo sapiens | CTGGGCATGGTTACG |
| NO: 3380 | ceruloplasmin | GTGCTCTGATCAACA |
| | (CP), RefSeqGene on chromosome 3 | TCTTTCCATGCACAA |
| | | GATTGGGGCTGAGTT |
| SEQ ID | GI 299782605 NG_023187 Homo sapiens | GATTCCCCACTCCCC |
| NO: 3381 | major histocompatibility | TGAGTTTCACTTCTT |
| | complex, class 1, B | CTCCCAACTTGTGTC |
| | (HLA-B), RefSeqGene on chromosome 6 | GGGTCCTTCTTCCAG |
| SEQ ID | GI 189409110 NM_153000 Homo sapiens | GTTTGAAGTTTCTGC |
| NO: 3382 | APC down-regulated 1 | TTTGAACTCCGTCCA |
| | (APCDD1), mRNA | GCCTGATCCCTGGCC |
| | | TGAGCAACTTCACAA |
| SEQ ID | GI 1027853651 NM_182828 Homo sapiens | AGACCTTTGGCTGCC |
| NO: 3383 | growth differentiation factor 7 | AGAATCTAGAACCAT |
| | (GDF7), mRNA | GGCTGACCAAAGCGA |
| | | GACTTCCGAGTAAGG |
| SEQ ID | GI 338797748 NM_006743 Homo sapiens | AACTGCCATGTCCTC |
| NO: 3384 | RNA binding motif | TGAAGAAGGAAAGCT |
| | (RNP1, RRM) protein 3 | CTTCGTGGGAGGGCT |
| | (RBM3), mRNA | CAACTTTAACACCGA |
| SEQ ID | GI 392055950 JQ867454 Homo sapiens | ATGGCCACTGCTTTG |
| NO: 3385 | ERBB receptor feedback inhibitor 1 | CAGAAAATGGTCCAT |
| | (ERRFIl) gene, complete cds | CTCAAAAGTCCAGCT |
| | | TGCCCCCTCTTCTTA |
| SEQ ID | GI 225735572 NG_011717 Homo sapiens | TGCCATAGCTGGCAG |
| NO: 3386 | alpha-2-macroglobulin | AAACACAGAAGCACT |
| | (A2M), RefSeqGene on chromosome 12 | ACCAAAATCCACATG |
| | | CTGGTCACTGTGAGC |
| SEQ ID | GI 223972657 NG_009817 Homo sapiens | ATGGTGCGTTTTTGC |
| NO: 3387 | E1A binding protein p300 | CTGGTTCTTGAACTC |
| | (EP300), RefSeqGene on chromosome 22 | GGAGAAGAGAGACTA |
| | | GGCTTCTCCTGCTCC |
| SEQ ID | GI 1657840 U73304 Human | TCCCACACATTGATG |
| NO: 3388 | CB1 cannabinoid receptor | AAACCTACCTGATGT |
| | (CNR1) gene, complete cds | TCTGGATCGGGGTCA |
| | | CCAGCGTACTGCTTC |
| SEQ ID | GI 593023777 KJ145927 Homo sapiens | CCTCTCAGCTTCCTT |
| NO: 3389 | lamin A/C | CCAGTTCTTGTGTTC |
| | (LMNA) gene, complete cds | TGTGACCCCTTTTCC |
| | | TCATCTCTGCCTGCT |
| SEQ ID | GI 224809232 NG_011437 Homo sapiens | ACCAGACATACATGT |
| NO: 3390 | arachidonate 5-lipoxygenase | GTTTTTGGCCCAAAG |
| | (ALOX5), RefSeqGene on chromosome 10 | CAGGGGTCTTTAGTT |
| | | GGAATGCCTCCTCCC |
| SEQ ID | GI 197245396 NG_008399 Homo sapiens | GGCAAACGTACATGC |
| NO: 3391 | aspartoacylase | TTCCAAGGCCAAGGA |
| | (ASPA), RefSeqGene on chromosome 17 | GGAAACACAGTATTT |
| | | AGCCACCGGGAACTT |
| SEQ ID | GI 269954683 NG_015990 Homo sapiens | GCAGTACTTAATGGA |
| NO: 3392 | C-C motif chemokine ligand 5 | CAGAGCACTGAAGTG |
| | (CCL5), RefSeqGene on chromosome 17 | GGAGTGAGGGCAACT |
| | | GGGTTCTGATCCCAA |
| SEQ ID | GI 816197633 NG_008177 Homo sapiens | CCCTTGCCTTTGAAA |
| NO: 3393 | GATA binding protein 4 | AACGCTTCATTCTCT |
| | (GATA4), RefSeqGene on chromosome 8 | GCTAGCTAGGCATTC |
| | | TGGGATGGGTGGGTC |
| SEQ ID | GI 577019604 NG_033916 Homo sapiens | AGAACTTTGCCTGTA |
| NO: 3394 | growth differentiation factor 2 | CTGTAGCTCAAGCCA |
| | (GDF2), RefSeqGene on chromosome 10 | CGCTAAAGCCAAACC |
| | | GTTGCCAACCAAGAA |
| SEQ ID | GI 212549732 NG_008981 Homo sapiens | GCGTCAGCAGCTTGC |
| NO: 3395 | growth differentiation factor 6 | AAAGCTCAGATCTCA |
| | (GDF6), RefSeqGene on chromosome 8 | ACAGAGGCCTGACTC |
| | | AGGATGAGTATCTCC |
| SEQ ID | GI 197245401 NG_008401 Homo sapiens | TCCCTCCATAGGCTG |
| NO: 3396 | glial fibrillary acidic protein | TTCTAAGGGGAGCCT |
| | (GFAP), RefSeqGene on chromosome 17 | TGGTACCAAAGCCAC |
| | | TAGATGGGATCCTAA |
| SEQ ID | GI 1135449810 NG_052673 Homo sapiens | CCTAACGGAAAGCAC |
| NO: 3397 | 0-6-methylguanine-DNA methyltransferase | AGAAAACAAAACCAC |
| | (MGMT), RefSeqGene on chromosome 10 | CAACTCTGTGTTCGC |
| | | CGAGGAAGGCAGGAA |
| SEQ ID | GI 345478700 NG_029900 Homo sapiens | GGGCTGCAGAGATGT |
| NO: 3398 | p21 | CCCCCTCAACACTGT |
| | (RAC1) activated kinase 1 | AGAAGAGAGGAGAAC |
| | (PAK1), RefSeqGene on chromosome 11 | TTGGGAAGTAGACCT |
| SEQ ID | GI 378786656 NG_032107 Homo sapiens | CCATCGGCTTCCCTG |
| NO: 3399 | TNF receptor superfamily member 10a | GGTCTTAGGTTTTTG |
| | (TNFRSF10A), RefSeqGene on chromosome 8 | GGCTGGATTAAGTCC |
| | | TGCTTCCAGCATTTC |
| SEQ ID | GI 815930042 NM_001308319 Homo sapiens | CTATGTTCCCCATGC |
| NO: 3400 | chromodomain helicase | TGCTGTCAGGAATGG |
| | DNA binding protein 9 | CTGGATTACCAAATC |
| | (CHD9), transcript variant 1, mRNA | TGTTGGGCATGGGAG |
| SEQ ID | GI 4455117 AF125158 Homo sapiens | GAGTGTGGTTTCGGC |
| NO: 3401 | zinc finger DNA binding protein 99 | CAATCTGTTACCTCA |
| | (ZNF281) mRNA, complete cds | GTGTTGCCATCTTCA |
| | | TTGCCAAAGCCTCCT |
| SEQ ID | GI 117606545 EF064747 Homo sapiens | GTGACCAGTGTGGGC |
| NO: 3402 | class 11, major histocompatibility | AAATGCTGGAGGTTC |
| | complex, transactivator | TCAGATTTGAAGCTA |
| | (CIITA) gene, complete cds | ATGGACCCGTTTGCT |
| SEQ ID | GI 166706816 EU332867 Homo sapiens | GTTACTGGATCACAA |
| NO: 3403 | protein kinase C, epsilon | GCAGCCAGGGACAAT |
| | (PRKCE) gene, complete cds | GTGCAGTTGTTGGTC |
| | | CTGGGTCTTTGACAG |
| SEQ ID | GI 312433967 NG_027936 Homo sapiens | ATGGACCACGCGTTT |
| NO: 3404 | pannexin 1 | GGCAGGTACAGACTA |
| | (PANX1), RefSeqGene on chromosome 11 | TTAATTCTTCCCTCC |
| | | ATCTGGTGAGGGCAG |
| SEQ ID | GI 343432618 NG_029713 Homo sapiens | TTCCCAGGCATGAGA |
| NO: 3405 | synoviolin 1 | TGATGATGGTCTGGA |
| | (SYVN1), RefSeqGene on chromosome 11 | CTAGGACACTGGCAG |
| | | TGAAAATGGCAAGGA |
| SEQ ID | GI 224809355 NG_011515 Homo sapiens | CTTACTAACCACCTA |
| NO: 3406 | uncoupling protein 3 | GCCACAGGGAATCCT |
| | (UCP3), RefSeqGene on chromosome 11 | TGCACCTTGGTGTGC |
| | | TACCAGAGAAGTGGG |
| SEQ ID | GI 156104863 NM_003501 Homo sapiens | AATACCAGTGCTTGA |
| NO: 3407 | acyl-CoA oxidase 3, pristanoyl | GTATCCAATGCAGCA |
| | (ACOX3), transcript variant 1, mRNA | ATGGCGCTTGCTTCC |
| | | ATATCTGGCAGCTGT |
| SEQ ID | GI 740086238 NM_017921 Homo sapiens | TCGATGAAATTGCTG |
| NO: 3408 | NPL4 homolog, ubiquitin | CCAAACTTGGCCTGC |
| | recognition factor | GGAAGGTTGGCTGGA |
| | (NPLOC4), transcript variant 1, mRNA | TATTTACAGACCTCG |
| SEQ ID | GI 190014585 NM_021144 Homo sapiens | GCCAAGATGAAAGGT |
| NO: 3409 | PC4 and SFRS1 interacting protein 1 | TATCCCCATTGGCCA |
| | (PSIP1), transcript variant 1, mRNA | GCTCGAGTAGACGAA |
| | | GTTCCTGATGGAGCT |
| SEQ ID | GI 15021772 AF400075 Homo sapiens | CCAATTGTCTACACA |
| NO: 3410 | coagulation factor II | ATTGTGTTTGTGGTG |
| | (thrombin) receptor-like 1 | GGTTTGCCAAGTAAC |
| | (F2RL1) gene, complete cds | GGCATGGCCCTGTGG |
| SEQ ID | GI 32187528 AY324876 Homo sapiens | GACCTCTCACAGCCG |
| NO: 3411 | lactoperoxidase | CAGACACTAGAGGAG |
| | (LPO) gene, complete cds | TTGAACACAGTGCTG |
| | | AAGAGCAAGATGCTG |
| SEQ ID | GI 205361192 NG_008140 Homo sapiens | TTCCCAATTTCAGGA |
| NO: 3412 | aminoadipate-semialdehyde synthase | GCACGGACTCAGCAC |
| | (AASS), RefSeqGene on chromosome 7 | CCAATGTGGATGGAA |
| | | GAGGTCTCCCTTATT |
| SEQ ID | GI 379991110 NG_032143 Homo sapiens | CTGATGTGCATTCCT |
| NO: 3413 | aconitase 2 | GTGTTGCCCACTTAG |
| | (ACO2), RefSeqGene on chromosome 22 | ACAGAGTCCACCTGA |
| | | GACTTCTCTGCCAGG |
| SEQ ID | GI 219521817 NG_009217 Homo sapiens | GGGAGAGTTATCCTG |
| NO: 3414 | cathepsin B | GTTCTGGCTGTGTCC |
| | (CTSB), RefSeqGene on chromosome 8 | TCCCCATTTTGGCCA |
| | | TCTTATGAGCTCCTT |
| SEQ ID | GI 751130526 NG_041830 Homo sapiens | TATGTGTTGAAGGCA |
| NO: 3415 | DnaJ heat shock protein family | GGCCAGGGTCATATG |
| | (Hsp40) member C3 | GAGAGTGGAGGGTGA |
| | (DNAJC3), RefSeqGene on | GGAGTGTGACAGATA |
| | chromosome [symbol] | |
| SEQ ID | GI 237874184 NG_012166 Homo sapiens | ACCCTGAAATGACTC |
| NO: 3416 | T-box 21 | CAGCTGAGTTACAGC |
| | (TBX21), RefSeqGene on chromosome 17 | AAGGCCACATGTCCT |
| | | ACCTTCAGCACTCAG |
| SEQ ID | GI 1010228574 NM_001321573 Homo sapiens | CGCTCGGGGTCACCA |
| NO: 3417 | calcium/calmodulin dependent | ACAGTACCCATCAAG |
| | protein kinase II delta | CCACCCTGTATTCCA |
| | (CAMK2D), transcript variant 14, [symbol] | AATGGGAAAGAAAAC |
| SEQ ID | GI 71648785 DQ147772 Homo sapiens | CCAGAGATGAGATTC |
| NO: 3418 | apolipoprotein B mRNA editing | TTCCACTGGTTCAGC |
| | enzyme, catalytic polypeptide-like 3G | AAGTGGAGGAAGCTG |
| | (APOBEC3G) gene, comp | CATCGTGACCAGGAG |
| SEQ ID | GI 14423572 AF388025 Homo sapiens | TCTGTTTCTTGGCAC |
| NO: 3419 | colony stimulating factor 3 | GCTGGGGTAAATTAG |
| | (granulocyte) | GACAGAAGGTGACAG |
| | (CSF3) gene, complete cds | TCTTGGGTGTGGTCA |
| SEQ ID | GI 189217867 NG_007938 Homo sapiens | GGCAACATACAGTCT |
| NO: 3420 | cytochrome P450 family 3 | GCCCATCTGTCACCA |
| | subfamily A member 5 | ATCCTTTTCTACTCT |
| | (CYP3A5), RefSeqGene on chromosome 7 | GCACACTCCTGGGGA |
| SEQ ID | GI 190358507 NG_008042 Homo sapiens | TCTCTGGGCTTTATA |
| NO: 3421 | 2,4-dienoyl-CoA reductase 1 | ACCTCCTGGGAAGAA |
| | (DECR1), RefSeqGene on chromosome 8 | CTGGAGGTCTGTCAT |
| | | GGAGTCCCCTGAGTG |
| SEQ ID | GI 798958230 NG_042077 Homo sapiens | TGATACAGGCCACCC |
| NO: 3422 | enoyl-CoA hydratase, short chain 1 | ATCATCCAGTTGATG |
| | (ECHS1), RefSeqGene on chromosome 10 | ACATGGTGAGTGACC |
| | | AAGATGGGTGCATCT |
| SEQ ID | GI 298919225 NG_023030 Homo sapiens | ATAGGAAGCTGGGAC |
| NO: 3423 | heme oxygenase 1 | CAGAGAAAGCAAGTG |
| | (HMOX1), RefSeqGene on chromosome 22 | GCTCACCCATGGTCA |
| | | CACAGTTAAGGACTG |
| SEQ ID | GI 269954724 NG_016003 Homo sapiens | GGGCACCAACCTTCT |
| NO: 3424 | interferon alpha and | GGGCTAGAGAAATTG |
| | beta receptor subunit 2 | CCCAGGCTTTACAGA |
| | (IFNAR2), RefSeqGene on chromosome 21 | ACAAGCTGGAATCAG |
| SEQ ID | GI 723428434 NG_041793 Homo sapiens | GCATCACTACTCTCT |
| NO: 3425 | inhibitor of nuclear factor | GCTGGCTTCCTTGAG |
| | kappa B kinase subunit beta | GAACTATGCTACCCT |
| | (IKBKB), RefSeqGene on chromosome [symbol] | CCCTCTCTCCAGACC |
| SEQ ID | GI 654825055 NG_028206 Homo sapiens | TGCTTTTCATTCCAC |
| NO: 3426 | prostaglandin-endoperoxide synthase 2 | TCTTTTCCTGTCCTT |
| | (PTGS2), RefSeqGene on chromosome 1 | AAGTGGGACAAGATC |
| | | ACTGGGGGGGGGCTA |
| SEQ ID | GI 345478715 NG_029910 Homo sapiens | GAATGTCGAGAGGGG |
| NO: 3427 | TNF receptor superfamily member 25 | GTGAAGACATTTCTC |
| | (TNFRSF25), RefSeqGene on chromosome 1 | AACTTCTCGGCCGGA |
| | | GTTTGGCTGAGATCG |
| SEQ ID | GI 164695818 AK315628 Homo sapiens | CCTGCCTGAAGCCAT |
| NO: 3428 | cDNA, FU96713, Homo sapiens | GACCCGCTTCTCCTA |
| | ankyrin repeat and SOCS box-containing 2 | TGCAGAGTACTTTTC |
| | (ASB2), mRNA | CCTCTTTCACTCCTG |
| SEQ ID | GI 520262262 NM_002872 Homo sapiens | CTTAGCAGATGGCTG |
| NO: 3429 | ras-related C3 botulinum | CAGAGCTTCGTTGAT |
| | toxin substrate 2 | GGTCTTTTCTGTACT |
| | (rho family, small GTP | GGAGGCCTCCTGAGG |
| | binding protein Rac2) | |
| | (RA [symbol] | |
| SEQ ID | GI 966209818 NM_014031 Homo sapiens | CAGCTGTGATTAGTC |
| NO: 3430 | solute carrier family 27 member 6 | AGCTGCAGGTTTTAA |
| | (SLC27A6), transcript variant 1, mRNA | GGGGTTCTGCTGTCC |
| | | TGTGGGCTTTTGGTT |
| SEQ ID | GI 117606579 EF064764 Homo sapiens | GAAATTGGGCCTCAG |
| NO: 3431 | chemokine | GCTACAGCTGATGCT |
| | (C-C motif) ligand 22 | TAGTTGTTCTGAATC |
| | (CCL22) gene, complete cds | CCCGCTCCTAGAGCT |
| SEQ ID | GI 1036032812 AH005421 Homo sapiens | TCAGTATGAGCTGCT |
| NO: 3432 | CD19 | TCCTGTCCCTCTACC |
| | (CD19) gene, complete cds | TCTCACTGTCTTCTC |
| | | TCTCTCTGCGGGTCT |
| SEQ ID | GI 117606529 EF064739 Homo sapiens | CTGAGGCCTTGAAGG |
| NO: 3433 | Fas ligand | CTGTTATCAGAAAAT |
| | (TNF superfamily, member 6) | TGTGGGCGGAAACTT |
| | (FASLG) gene, complete cds | CCAGGGGTTTGCTCT |
| SEQ ID | GI 1139695586 NG_052809 Homo sapiens | AGGAGGGGCAAAATT |
| NO: 3434 | B-cell CLL/lymphoma 3 | GCTCCCCTCAATTGA |
| | (BCL3), RefSeqGene on chromosome 19 | GAATCTAGACAGAGG |
| | | AGGGAGTCATGCTGC |
| SEQ ID | GI 974987494 NG_011716 Homo sapiens | TGGACCACGAAGGGA |
| NO: 3435 | transcription factor 4 | AAGCAGAGGTAAGGT |
| | (TCF4), RefSeqGene on chromosome 18 | CAGAGTCTCCCCAAA |
| | | GTGGTCTATCCTGAG |
| SEQ ID | GI 572873826 NM_001130080 Homo sapiens | CTTGTGGCTACTCTG |
| NO: 3436 | interferon alpha inducible protein 27 | CAGTCACTGGGAGCA |
| | (IFI27), transcript variant 1, mRNA | ACTGGACTCTCCGGA |
| | | TTGACCAAGTTCATC |
| SEQ ID | GI 206725550 NM_004958 Homo sapiens | TTGGCCCTCACCTCA |
| NO: 3437 | mechanistic target of rapamycin | CAAGACATCGCTGAA |
| | (MTOR), mRNA | GTCACACAGACCCTC |
| | | TTAAACTTGGCTGAA |
| SEQ ID | GI 195222745 NM_018290 Homo sapiens | GAGAAGAACCAGGAT |
| NO: 3438 | phosphoglucomutase 2 | CGCAGTGCTCTCAAA |
| | (PGM2), mRNA | GACACGTACATGTTG |
| | | TCCAGCACCGTCTCC |
| SEQ ID | GI 590122055 NM_021649 Homo sapiens | GGCCCCAGGAGATCT |
| NO: 3439 | toll like receptor adaptor molecule 2 | GATCACACTTCCTCC |
| | (TICAM2), mRNA | TGAGGCACCTCTCAT |
| | | GGATGTTGCAATAAG |
| SEQ ID | GI 1025812269 NG_047054 Homo sapiens | TCTCCTAGCCTGCTT |
| NO: 3440 | glutathione peroxidase 5 | TGTGCAAACAAGTCC |
| | (GPX5), RefSeqGene on chromosome 6 | CAAGCAGGAGAAGAT |
| | | GAAGGTGAGTGAGCT |
| SEQ ID | GI 1140392578 NG_052831 Homo sapiens | CAGACAGGAAGGGAT |
| NO: 3441 | glutamate metabotropic receptor 5 | CTCAACTGCTACCCT |
| | (GRM5), RefSeqGene on chromosome 11 | CTGCATGAGTTTGCC |
| | | TTCTTGGGCTCAGAG |
| SEQ ID | GI 300116282 NG_023271 Homo sapiens | ATGAGAAAGGGTGAG |
| NO: 3442 | regenerating family member 1 alpha | GTGGGGGCCCTAAGT |
| | (REGIA), RefSeqGene on chromosome 2 | TTCTGCTCTTCCAAG |
| | | GCTCCTCCAATATTT |
| SEQ ID | GI 193083137 NM_005512 Homo sapiens | GAAGAGTGAGGACTG |
| NO: 3443 | leucine rich repeat containing 32 | ACTCAAGGTCACACA |
| | (LRRC32), transcript variant 1, mRNA | GTGATCCGGGATCCC |
| | | AGAACTCTGGTCTCC |
| SEQ ID | GI 225579056 NM_003049 Homo sapiens | AAGGGAGGGATGATC |
| NO: 3444 | solute carrier family 10 member 1 | ATCATTCTCTTGTGC |
| | (SLC10A1), mRNA | AGTGTGGCCGTCACA |
| | | GTTCTCTCTGCCATC |
| SEQ ID | GI 313661479 NM_145725 Homo sapiens | GGCCGTTTAAGCAGA |
| NO: 3445 | TNF receptor associated factor 3 | AAGTGACACTCATGC |
| | (TRAF3), transcript variant 1, mRNA | TGATGGATCAGGGGT |
| | | CCTCTCGACGTCATT |
| SEQ ID | GI 67515408 DQ080435 Homo sapiens | GAGCCAAGGTCTGTG |
| NO: 3446 | calcitonin/calcitonin- | GGCTCCTAGAACTTC |
| | related polypeptide, alpha | GAACTGGCGCAGGTG |
| | (CALCA) gene, complete cds | GTTTATCTCATTCTT |
| SEQ ID | GI 349585381 NG_030049 Homo sapiens | TGATGTTTCCTTCCT |
| NO: 3447 | checkpoint kinase 1 | GCCCTCCACCCTACC |
| | (CHEK1), RefSeqGene on chromosome 11 | TGATGAGGGGCCTTG |
| | | CTTTACTTTCATCTC |
| SEQ ID | GI 283945491 NG_016362 Homo sapiens | TGGTGGGAAATTTGT |
| NO: 3448 | C-X3-C motif chemokine receptor 1 | TGGTAGTGTTTGCCC |
| | (CX3CR1), RefSeqGene on chromosome 3 | TCACCAACAGCAAGA |
| | | AGCCCAAGAGTGTCA |
| SEQ ID | GI 982742415 NM_020530 Homo sapiens | ACAGATCTCATGCAG |
| NO: 3449 | oncostatin M | GACACCAGCAGACTC |
| | (OSM), transcript variant 1, mRNA | CTGGACCCCTATATA |
| | | CGTATCCAAGGCCTG |
| SEQ ID | GI 153791376 NM_024310 Homo sapiens | CAGTGCCTTTTTGCT |
| NO: 3450 | pleckstrin homology and | GGACACTGTGTCCTT |
| | FYVE domain containing 1 | ATGGCTTCACTGCAG |
| | (PLEKHF1), mRNA | GTAATGCCTTTCCCT |
| SEQ ID | GI 48762927 NM_002881 Homo sapiens | TGAATGCATGTGTCT |
| NO: 3451 | RAS like proto-oncogene B | GTCTGATCAGCATCA |
| | (RALB), mRNA | CTGCACACGGAGGTC |
| | | TAGTGAGCCTCTTGC |
| SEQ ID | GI 32140476 AY316305 Homo sapiens | TTGGTAGGGCACAAA |
| NO: 3452 | glutathione transferase zeta 1 | TGAGCACCTCTCTGG |
| | (maleylacetoacetate isomerase) | AAGGCATCATGCTAG |
| | (GSTZ1) gene, complete cds | GCGTTAGGGATATGG |
| SEQ ID | GI 46091142 AB110934 Homo sapiens | AAGTATACCCCTCTG |
| NO: 3453 | MRPS18B gene for MRPS18B protein, | CTAGGGCAGATGCAG |
| | complete cds, cell_line: LKT3 | CTTCATAGTCCTTGT |
| | | TCAGCACTGCACAGC |
| SEQ ID | GI 3403164 AFO27148 Homo sapiens | TGGGACTGAGGTGTG |
| NO: 3454 | myogenic determining factor 3 | GACGTACCTAACTTA |
| | (MYOD1) gene, complete cds | GAATATGAGCAGGTG |
| | | GGACGCATCTGTGGT |
| SEQ ID | GI 209529670 NG_008721 Homo sapiens | CTTCGAGGACCTGTA |
| NO: 3455 | LIF, interleukin 6 family cytokine | AGATACCAGCTGCCA |
| | (LIF), RefSeqGene on chromosome 22 | AGGTCTCTGTTGCCC |
| | | AGGAATGTCCTGTTG |
| SEQ ID | GI 240255451 NG_012330 Homo sapiens | GCAGCTTGATGTGTC |
| NO: 3456 | MYB proto-oncogene, transcription factor | TGCCATTGGCACTGT |
| | (MYB), RefSeqGene on chromosome 6 | GCAACACCACGACTT |
| | | TTCGTGCAAGATTTT |
| SEQ ID | GI 1140398720 NG_052834 Homo sapiens | GTCCCTGCGCCATGT |
| NO: 3457 | TNF receptor superfamily member 9 | ATTCAATACGTCGTC |
| | (TNFRSF9), RefSeqGene on chromosome 1 | TTTCTCCTGCGGGTC |
| | | TGCAATCTCACCTAC |
| SEQ ID | GI 116006947 NM_001285 Homo sapiens | AGAACATCCTCGGGA |
| NO: 3458 | chloride channel accessory 1 | GGCTCATTTGTGGCT |
| | (CLCA1), mRNA | TCTGATGTCCCAAAT |
| | | GCTCCCATACCTGAT |
| SEQ ID | GI 306140488 NM_030956 Homo sapiens | ATACTTGGAATGGCC |
| NO: 3459 | toll like receptor 10 | CAAGGATAGGCGTAA |
| | (TLR10), transcript variant 1, mRNA | ATGTGGGCTTTTCTG |
| | | GGCAAACCTTCGAGC |
| SEQ ID | GI 1015809708 NM_003807 Homo sapiens | CACATTTTCAGAAGC |
| NO: 3460 | TNF superfamily member 14 | CTCTGGAAAGTCGTG |
| | (TNFSF14), transcript variant 1, mRNA | CACAGCCCAGGAGTG |
| | | TTGAGCAATTTCGGT |
| SEQ ID | GI 167887714 EF445034 Homo sapiens | TGAAGCCAGGTACGA |
| NO: 3461 | transgelin | GCAAGATGAGAGTGG |
| | (TAGLN) gene, complete cds, alternatively spliced | GTTATCTCTGGAGTG |
| | | ACAGAGGCTGGTCTG |
| SEQ ID | GI 283837772 NG_016339 Homo sapiens | CGTTCTGTGACTTCT |
| NO: 3462 | A-Raf proto-oncogene, serine/threonine kinase | GCCTTAAGTTTCTGT |
| | (ARAF), RefSeqGene on chromosome X | TCCATGGCTTCCGTT |
| | | GCCAAACCTGTGGCT |
| SEQ ID | GI 1032528813 NG_050736 Homo sapiens | CTTTTTACTCCCACC |
| NO: 3463 | complement C3a receptor 1 | AACGCCATGTACCTG |
| | (C3AR1), RefSeqGene on chromosome 12 | TGCCATTTAGTTGTG |
| | | TTGGGCTCACCTCCC |
| SEQ ID | GI 1028908919 NG_050629 Homo sapiens | ATTTTTGTTGCGCCT |
| NO: 3464 | neurotrophin 3 | ACGAACCAAATGCCC |
| | (NTF3), RefSeqGene on chromosome 12 | CCGTCACTCTCTGCC |
| | | ATTGTTTAGCAGTCT |
| SEQ ID | GI 238637280 NG_012233 Homo sapiens | CCATCAGGGTGAGGG |
| NO: 3465 | pyruvate dehyrogenase | CATCTCAGACCCACA |
| | phosphatase catalytic subunit 1 | TGCAGGTTTATTTTA |
| | (PDP1), RefSeqGene on chromosom [symbol] | AGCTTCTTGGCTGGC |
| SEQ ID | GI 166795249 NM_006845 Homo sapiens | GGGAGCTGGAGGAGA |
| NO: 3466 | kinesin family member 2C | AGGCTATGGAAGAGC |
| | (KIF2C), transcript variant 1, mRNA | TCAAGGAGATCATAC |
| | | AGCAAGGACCAGACT |
| SEQ ID | GI 558472802 NM_001042600 Homo sapiens | ATCCCAGCCTGGGCT |
| NO: 3467 | mitogen-activated protein kinase | GAATCGAGGCCTGAT |
| | kinase kinase kinase 1 | CCTGGATCTTCTTGA |
| | (MAP4K1), transcript variant 1, | CAAACTGAAGAATCC |
| SEQ ID | GI 542029075 NM_006440 Homo sapiens | TCACGTGAAATCCTT |
| NO: 3468 | thioredoxin reductase 2 | GAACTGGGGCCACCG |
| | (TXNRD2), transcript variant 1, mRNA | TGTCCAGCTTCAGGA |
| | | CAGAAAAGTCAAGTA |
| SEQ ID | GI 1213447 U48869 Human cdk-inhibitor p57/KIP2 | AATCGCCGTGGTGTT |
| NO: 3469 | (CDKNIC) gene, complete cds | GTTGAAACTGAAAAT |
| | | ACTACATTATGCTAA |
| | | TCGCGGCCGGGCCCG |
| SEQ ID | GI 22001412 AF531102 Homo sapiens | AAACACCAGTTACTG |
| NO: 3470 | interleukin 1 receptor, type 1 | TCACCAGCCACTAAG |
| | (IL1R1) gene, complete cds | GAGAAACTGCAAAGA |
| | | GAGGCTCACGTGCCT |
| SEQ ID | GI 4588599 AF111163 Homo sapiens | CTCTTTCCTTGGCAT |
| NO: 3471 | pyrin | CACCTGCTAGATCTA |
| | (MEFV) gene, complete cds | TGTCTGTGCAGGAGG |
| | | AACAGCACAAGGCCA |
| SEQ ID | GI 238018114 NG_012216 Homo sapiens | GTGAAGCTCTTTTGC |
| NO: 3472 | B-cell CLL/lymphoma 10 | ATAAGGGGGCTTGAG |
| | (BCL10), RefSeqGene on chromosome 1 | GAAGCTCAACAGCTG |
| | | AAAGCACAACCTGGA |
| SEQ ID | GI 1033837679 NG_008338 Homo sapiens | CCTTCAGTTGTCAAG |
| NO: 3473 | cytochrome P450family 7 subfamily B member 1 | TCCAACAGCTGCACT |
| | (CYP7B1), RefSeqGene on chromosome 8 [symbol] | GCAACTGTGAAAAGC |
| | | CTTTCTCCCTGCTGA |
| SEQ ID | GI 237820700 NG_012083 Homo sapiens | CAGTTGTACAGGTTG |
| NO: 3474 | intercellular adhesion molecule 1 | TACACTGCAGGAGAG |
| | (ICAM1), RefSeqGene on chromosome 19 | TGCCTGGCAAAAAGA |
| | | TCAAATGGGGCTGGG |
| SEQ ID | GI 254826724 NG_012488 Homo sapiens | TCTTTTGTGGGCCTG |
| NO: 3475 | prostaglandin E synthase 2 | GTGGGAGCAACTCTG |
| | (PTGES2), RefSeqGene on chromosome 9 | ACCTCAGGGATTGCT |
| | | TTCTCGTCTATCATG |
| SEQ ID | GI 1049480218 NG_051308 Homo sapiens | TCCACCTCCCCTTCC |
| NO: 3476 | ras homolog family member A | ATTAGGGTTCTTTCT |
| | (RHOA), RefSeqGene on chromosome 3 | TTTGGGCACCCTCAT |
| | | ACCCTTGCCATAGTT |
| SEQ ID | GI 167614484 NM_006111 Homo sapiens | GGAGGAACCGAAAGC |
| NO: 3477 | acetyl-CoA acyltransferase 2 | ATGAGCCAAGCTCCC |
| | (ACAA2), mRNA | TACTGTGTCAGAAAT |
| | | GTGCGTTTTGGAACC |
| SEQ ID | GI 53759124 NM_001295 Homo sapiens | GGAATTCACTCACCA |
| NO: 3478 | C-C motif chemokine receptor 1 | CACCTGCAGCCTTCA |
| | (CCR1), mRNA | CTTTCCTCACGAAAG |
| | | CCTACGAGAGTGGAA |
| SEQ ID | GI 665505917 NM_006145 Homo sapiens | CCATGGAGACCCTCA |
| NO: 3479 | DnaJ heat shock protein family | TGCCATGTTTGCTGA |
| | (Hsp40) member B1 | GTTCTTCGGTGGCAG |
| | (DNAJB1), transcript variant 1, mRNA | AAATCCCTTTGACAC |
| SEQ ID | GI 41353192 AY486465 Homo sapiens | GGAGATGCACAGTGA |
| NO: 3480 | glutathione transferase kappa 1 | GAGAAAAGAGAGCAT |
| | (GSTK1) gene, complete cds | CTTAGAAACAGGAAG |
| | | GCACCTGGGGCCACA |
| SEQ ID | GI 37654535 AY434089 Homo sapiens | AGGGCTTTTTTCTGT |
| NO: 3481 | insulin-like growth factor binding protein 1 | GTCAATTAAAGAGCC |
| | (IGFBP1) gene, complete cds | TGGCCAGCTGGACCT |
| | | GGGCTGTCTlTTTGA |
| SEQ ID | GI 983173900 NG_009770 Homo sapiens | AGAAGTGCCAGCCTT |
| NO: 3482 | hexosaminidase subunit beta | ACTGTCTGCAACAGT |
| | (HEXB), RefSeqGene on chromosome 5 | TCTGGGAGGTAGCAA |
| | | TAGCGCTTCTTCAGG |
| SEQ ID | GI 1047225539 NM_001201 Homo sapiens | CTCTTCCCCCACAAG |
| NO: 3483 | bone morphogenetic protein 3 | TAATCTCTCTACCCC |
| | (BMP3), mRNA | ATGCAGTGTGCACAC |
| | | ACACACACACACAGT |
| SEQ ID | GI 403420573 NM_182619 Homo sapiens | TCACCTTCATGGTCC |
| NO: 3484 | C-type lectin domain family 18 member A | AGGACACTCTCTGGA |
| | (CLEC18A), transcript variant 1, mRNA | AGTTCTGGGTCTCCC |
| | | CAAGAAGAGGAAGAC |
| SEQ ID | GI 402794166 NM_002382 Homo sapiens | ACCGAGGTTTCAATC |
| NO: 3485 | MYC associated factor X | TGCGGCTGACAAACG |
| | (MAX), transcript variant 1, mRNA | GGCTCATCATAATGC |
| | | ACTGGAACGAAAACG |
| SEQ ID | GI 1003088850 NM_007368 Homo sapiens | GCTGAGGGGTCAGTT |
| NO: 3486 | RAS p21 protein activator 3 | TTATGGCTGGGAGGT |
| | (RASA3), transcript variant 1, mRNA | GCAGAATTACACAGA |
| | | AGTCCCAGTCTGAGG |
| SEQ ID | GI 73858577 NM_003255 Homo sapiens | TGATCAGGGCCAAAG |
| NO: 3487 | TIMP metallopeptidase inhibitor 2 | CGGTCAGTGAGAAGG |
| | (TIMP2), mRNA | AAGTGGACTCTGGAA |
| | | ACGACATTTATGGCA |
| SEQ ID | GI 343790995 NG_029773 Homo sapiens | CCCAGAAGACCTTCA |
| NO: 3488 | activating transcription factor 6 | GGTGACTGGGCAGTC |
| | (ATF6), RefSeqGene on chromosome 1 | TTATCATGGGATATT |
| | | TCTTCTGGCCCTGCC |
| SEQ ID | GI 205277388 NG_008604 Homo sapiens | TCCAGTAGACAAAGC |
| NO: 3489 | polycystin 2, transient receptor | AGCCTTTGCTGGCTT |
| | potential cation channel | CTGAGCAAGCCTGTA |
| | (PKD2), RefSeqGene on chromosom [symbol] | GGCAAAGAAATGCAG |
| SEQ ID | GI 746815960 NM_004929 Homo sapiens | ATCCCACCTGCAGTC |
| NO: 3490 | calbindin 1 | ATCCCTCATCACAGC |
| | (CALB1), mRNA | CTCACAGTTTTTCGA |
| | | GATCTGGCTCCATTT |
| SEQ ID | GI 403420573 NM_182619 Homo sapiens | GGATCGACGGAGACT |
| NO: 3491 | C-type lectin domain family 18 member A | GCTTCATGGTGTCTT |
| | (CLEC18A), transcript variant 1, mRNA | CAGAGGCAGACACCT |
| | | ATTACAGAGCCAGGA |
| SEQ ID | GI 658309997 NM_000853 Homo sapiens | CTGCGCATCGTGGAT |
| NO: 3492 | glutathione S-transferase theta l | CTGATTAAAGGTCAG |
| | (GSTT1), transcript variant 1, mRNA | CACTTAAGCGATGCC |
| | | TTTGCCCAGGTGAAC |
| SEQ ID | GI 83699680 DQ314887 Homo sapiens | GTGATGCTGTGGCCT |
| NO: 3493 | v-rel reticuloendotheliosis viral oncogene | TCTCAGTGTCTCACA |
| | homolog B, nuclear factor of | TCAAGGGACGTGGGA |
| | kappa light polypeptide [symbol] | TGTCGTTATTCCCAA |
| SEQ ID | GI 32263624 AY325800 Homo sapiens | AAGGGTGATGGGGAC |
| NO: 3494 | stannin | GTCTTCGGGGTATTT |
| | (SNN) gene, complete cds | CACACTTAGGTCCTT |
| | | TCTGGAGAGAGAGGG |
| SEQ ID | GI 209977025 NG_008836 Homo sapiens | ACTTACAGCTGCAGG |
| NO: 3495 | angiotensinogen | CAGGAGCATGTCTTA |
| | (AGT), RefSeqGene on chromosome 1 | ATCTGCTTGGGCTGC |
| | | CATAAGTACCACAGA |
| SEQ ID | GI 1024336774 NG_046918 Homo sapiens | GAGATGAGCAGAGGA |
| NO: 3496 | DExD/H-box helicase 58 | GGAAAAGAGACTGGG |
| | (DDX58), RefSeqGene on chromosome 9 | AAAGAGTGCCACATA |
| | | TGTGGGCAAGGAGGC |
| SEQ ID | GI 267844874 NG_015840 Homo sapiens | GGATATGTGCTCAGC |
| NO: 3497 | interferon gamma | TTTCACTATTGCTGG |
| | (IFNG), RefSeqGene on chromosome 12 | GCCTGCCTCTCACTC |
| | | CTCTCATGTAAGCCC |
| SEQ ID | GI 239787846 NG_012306 Homo sapiens | AATTCGTGACACTGG |
| NO: 3498 | interferon regulatory factor 5 | TGTCACCATTCTCCT |
| | (IRF5), RefSeqGene on chromosome 7 | TTACAGATGGGAAAT |
| | | GGGGCTTCTGGGGGA |
| SEQ ID | GI 193290114 NG_008154 Homo sapiens | CTTGGCTGAGCACAG |
| NO: 3499 | insulin receptor substrate 2 | AGAATGGGATGTTTC |
| | (IRS2), RefSeqGene on chromosome 13 | ACGGGATGTCTCTGC |
| | | TGCGACAGTGGATAT |
| SEQ ID | GI 38261972 NM_024551 Homo sapiens | TCCCTTTGAGAAAGC |
| NO: 3500 | adiponectin receptor 2 | ATAGTCCAGCAGGAC |
| | (ADIPOR2), mRNA | AGTGGCCATTTGGAC |
| | | AGAAGCCCACTTAGT |
| SEQ ID | GI 388596695 NM_001227 Homo sapiens | GCCAGGCACTTTGAG |
| NO: 3501 | caspase 7 | TCTCAGTCTGATGAC |
| | (CASP7), transcript variant a, mRNA | CCACACTTCCATGAG |
| | | AAGAAGCAGATCCCC |
| SEQ ID | GI 117422442 NM_001242 Homo sapiens | CAGATGTGTGAGCCA |
| NO: 3502 | CD27 molecule | GGAACATTCCTCGTG |
| | (CD27), mRNA | AAGGACTGTGACCAG |
| | | CATAGAAAGGCTGCT |
| SEQ ID | GI 1024336732 NM_006218 Homo sapiens | TTTTTGAGAAGAGGG |
| NO: 3503 | phosphatidylinositol-4,5-bisphosphate | GAATGTGAGGGGAGG |
| | 3-kinase catalytic subunit alpha | GGGCAGAACAGGGAG |
| | (PIK3CA), mRNA | GAGTTGTTTGAATGA |
| SEQ ID | GI 1036029324 AHO13155 Homo sapiens | TTCCTAAGTCCTCCA |
| NO: 3504 | chromosome 19 poliovirus receptor related 2 | GGATGAAGGGAGCTT |
| | (PVRL2) gene, | GCATTTTGGCAATTT |
| | | GGAGTCTCCGGGAGT |
| SEQ ID | GI 290543602 GU130197 Homo sapiens | CCTGGTTTATTTTCT |
| NO: 3505 | isolate MIA060410 Duffy antigen/chemokine receptor | GGTGGCCTCATGGGG |
| | (DARC) gene, complete cds | TGGTTCTAGGACTGG |
| | | ATTTCCTGGTGAGGT |
| SEQ ID | GI 14488404 AF385628 Homo sapiens | AGACTGCTCCTGAGA |
| NO: 3506 | interleukin 8 | CTGAGAAAGGCAGCT |
| | (IL8) gene, complete cds | CTGACGGGATCTCAG |
| | | ATTTTAGCAGCAGGA |
| SEQ ID | GI 1101010097 NG_052015 Homo sapiens | AAGGAGCAGGCCGTG |
| NO: 3507 | adenosine deaminase, RNA specific B1 | GAGCTCTTGTCTATT |
| | (ADARB1), RefSeqGene on chromosome 21 | CCTTTTGTCAGAGAC |
| | | ATTGTGGAAGCCCTC |
| SEQ ID | GI 256220780 NG_012741 Homo sapiens | CTGACCAATTTGAAT |
| NO: 3508 | very low density lipoprotein receptor | GTGAGGATGGCAGCT |
| | (VLDLR), RefSeqGene on chromosome 9 | GCATCCATGGCAGCA |
| | | GGCAGTGTAATGGTA |
| SEQ ID | GI 608785172 KJ534783 Homo sapiens | TTTGACTATGACCCC |
| NO: 3509 | clone BZRAPl_iso-A_adult-A09 | GTGTCAATGTCGCCC |
| | benzodiazapine receptor peripheral- | AATCCTGATGCTGGA |
| | associated protein l isofo [symbol] | GAAGAAGAGCTTCCC |
| SEQ ID | GI 1006833939 NM_003978 Homo sapiens | CTTTTTCTCCTGCTC |
| NO: 3510 | proline-serine-threonine | CAGTGTCCGAGTGCT |
| | phosphatase interacting protein 1 | CAGTTCAGAGGAGGC |
| | (PSTPIP1), transcript variant 1, [symbol] | AAAGGAACAAGGGAA |
| SEQ ID | GI 19718735 NM_003265 Homo sapiens | CACTGTTAGCCATGA |
| NO: 3511 | toll like receptor 3 | AGTTGCTGACTGCAG |
| | (TLR3), mRNA | CCACCTGAAGTTGAC |
| | | TCAGGTACCCGATGA |
| SEQ ID | GI 117606575 EF064762 Homo sapiens | CGCACAGCCAAGTGA |
| NO: 3512 | chemokine | TGAAGAAGGTCTATG |
| | (C-C motif) ligand 27 | ACAAGCCCGAGGAAC |
| | (CCL27) gene, complete cds | CTGGGAGATTTCCTT |
| SEQ ID | GI 339639570 NG_029386 Homo sapiens | GAGTCAGTTAAGACC |
| NO: 3513 | complement component 4 binding protein beta | AGTTCCTTGCTGGGA |
| | (C4BPB), RefSeqGene on chromosome 1 | AGCCCTAACTCTGGA |
| | | GGGACAGAGACAGGT |
| SEQ ID | GI 429535873 NG_033024 Homo sapiens | ACCAGTGAGGTAAGA |
| NO: 3514 | colony stimulating factor 2 | AACAGCTGCCCCGGG |
| | (CSF2), RefSeqGene on chromosome 5 | ACCTTTCATACCACA |
| | | TTTGAACAGGGAGAG |
| SEQ ID | GI 237820700 NG_012083 Homo sapiens | TCCCAGAATCTCCCT |
| NO: 3515 | intercellular adhesion molecule 1 | TGGAGAATGTATGGA |
| | (ICAM1), RefSeqGene on chromosome 19 | GCCAGTGTCCTCACA |
| | | CCCCCATCCAAGATA |
| SEQ ID | GI 292658838 NG_016860 Homo sapiens | CTTCCTTCTCCTAGG |
| NO: 3516 | pyruvate dehydrogenase | TCCTGCGTTCAATTT |
| | (lipoamide) beta | CCTGGATGCTCCTGC |
| | (PDHB), RefSeqGene on chromosome 3 | TGTTCGTGTCACTGG |
| SEQ ID | GI 340523097 NG_029467 Homo sapiens | TCTCACCAGAATCCA |
| NO: 3517 | protein tyrosine kinase 2 | AGATTTGATCCCTTG |
| | (PTK2), RefSeqGene on chromosome 8 | CTGCCGAAAGCTCTG |
| | | CTGGTTTCTCTCCCA |
| SEQ ID | GI 262331561 NG_013364 Homo sapiens | CGCTGAGTGGGGCTT |
| NO: 3518 | transforming growth factor beta 1 | AGCATTTCACCAATG |
| | (TGFB1), RefSeqGene on chromosome 19 | AGGAAACAGGCCTCG |
| | | AGAAGGCAAGAAAAC |
| SEQ ID | GI 122937306 NM_001080454 Homo sapiens | ATCCAGCAGTTGTTG |
| NO: 3519 | acyl-CoA synthetase | AATCGGCTGTTGTCA |
| | medium chain family member 4 | GTAGTCCAGATCAAA |
| | (ACSM4), mRNA | TCCGCGGAGAGGTGG |
| SEQ ID | GI 336455032 NG_029156 Homo sapiens | GGCCTGAGCTAGAAG |
| NO: 3520 | betaine-homocysteine S-methyltransferase | GAAGCAAAGGAAATA |
| | (BHMT), RefSeqGene on chromosome 5 | GAAGGGCAGAAGTGG |
| | | AGCCTGCAGAGTGAG |
| SEQ ID | GI 193290109 NG_008150 Homo sapiens | CTGAGCTTCGCTTTG |
| NO: 3521 | checkpoint kinase 2 | TCTGTAGTTTTGAGC |
| | (CHEK2), RefSeqGene on chromosome 22 | TGAAGGGGGAGTGTG |
| | | TGGGCGACTCTCTTC |
| SEQ ID | GI 260593639 NG_013007 Homo sapiens | GTGGCTGCTTTCACA |
| NO: 3522 | C-reactive protein | GGCTGGCATTGAACA |
| | (CRP), RefSeqGene on chromosome 1 | CTTGCAAGCTTTTCT |
| | | AAGCACAAGGTGCAA |
| SEQ ID | GI 916539820 NG_042809 Homo sapiens | TCTGTGAAATGGGGA |
| NO: 3523 | potassium voltage-gated | GCCAGCCTTCTTGCT |
| | channel subfamily J member 12 | TGCTAAGGCCTCTTC |
| | (KCNJ12), RefSeqGene on chromos [symbol] | CAGCAAAACAGTTTC |
| SEQ ID | GI 237858713 NG_012098 Homo sapiens | TGTCACATTGAACTC |
| NO: 3524 | KIT ligand | TTCCCTCAGCGAAAG |
| | (KITLG), RefSeqGene on chromosome 12 | CATTTGCATTGCTTC |
| | | CCCAAGTCACTGCCA |
| SEQ ID | GI 47458816 NM_005622 Homo sapiens | CTGATGCTAGCTCGT |
| NO: 3525 | acyl-CoA synthetase medium chain family member 3 | GTCACCAGGAAGATG |
| | (ACSM3), transcript variant 1, mRNA | CTACGTCATGCCAAG |
| | | TGTTTTCAGCGCCTA |
| SEQ ID | GI 748585173 NM_198580 Homo sapiens | TCAGCCTCTGTCTCC |
| NO: 3526 | solute carrier family 27 member 1 | TTCCATCCCTGTCCC |
| | (SLC27A1), mRNA | TGTCTGGCCTTAACT |
| | | CTTCCCTCTTTTTCT |
| SEQ ID | GI 49066030 AY654286 Homo sapiens | TTCATGGTGCCTCAT |
| NO: 3527 | interferon | CCCTTGGGACCTGTG |
| | (alpha, beta and omega) receptor 1 | TTGCCACTGAGCCCT |
| | (IFNAR1) gene, complete cds | ATTAGCTCATATTCC |
| SEQ ID | GI 8308037 AF244139 Homo sapiens | TGTGTGCTCTTCCCC |
| NO: 3528 | integrin-linked kinase 1 | CTTTTCCCATGCCCT |
| | (ILK) gene, complete cds | GACACCAGTATCTCA |
| | | TTTGGAACTGACTGT |
| SEQ ID | GI 190194395 NG_008003 Homo sapiens | GGAGTGGTCAGAAAA |
| NO: 3529 | acyl-CoA dehydrogenase, short/branched chain | CTGCAACAAAAGGGG |
| | (ACADSB), RefSeqGene on chromosome 10 | ACTGAGGCCACTTTA |
| | | GGGAATTGGGGGTTG |
| SEQ ID | GI 224809264 NG_011470 Homo sapiens | GTGTGGTAGGTGAAT |
| NO: 3530 | nitric oxide synthase 2 | TGGAGGAGAGGACTT |
| | (NOS2), RefSeqGene on chromosome 17 | TGAAACCAGGCAGTC |
| | | TGGGCTATGGCAACT |
| SEQ ID | GI 429535866 NG_033016 Homo sapiens | ATTCCTCAGCTTCCG |
| NO: 3531 | receptor interacting serine/threonine kinase 2 | CCCTGCCAAAGTTAC |
| | (RIPK2), RefSeqGene on chromosome 8 | TGGATTTTCACCTGC |
| | | CTGTGGCTTTTCTTC |
| SEQ ID | GI 293597503 NG_016969 Homo sapiens | TGCAGTTGCAGAAGC |
| NO: 3532 | solute carrier family 6 member 2 | AGCCGTTTAAGACTG |
| | (SLC6A2), RefSeqGene on chromosome 16 | GAGTTTGAGTCTGAG |
| | | CTCTGCCTCTCAGTG |
| SEQ ID | GI 164695701 AK315511 Homo sapiens | AGCCAAACCTACCAG |
| NO: 3533 | cDNA, FLI96576, Homo sapiens | CAGCAATATGGACGG |
| | annexin A4 | AGCCTTGAAGATGAC |
| | (ANXA4), mRNA | ATTCGCTCTGACACA |
| SEQ ID | GI 237858728 NG_012105 Homo sapiens | TTTGAAAACATGGGC |
| NO: 3534 | ornithine decarboxylase 1 | GCTTACACTGTTGCT |
| | (ODC1), RefSeqGene on chromosome 2 | GCTGCCTCTACGTTC |
| | | AATGGCTTCCAGAGG |
| SEQ ID | GI 32815079 AY338491 Homo sapiens | TTGTTCTGTGCAAGG |
| NO: 3535 | cyclin B1 | CCATTTATGCAAAGC |
| | (CCNB1) gene, complete cds | AACCCCTCAAGTGGC |
| | | AGAGGAGCTGAGAAA |
| SEQ ID | GI 1036032792 AH002827 Homo sapiens | ATCTCAGAGAGATGG |
| NO: 3536 | hemopexin | AAGAATTTCCCCAGC |
| | (HPX) gene, complete cds | CCTGTGGATGCTGCA |
| | | TTCCGTCAAGGTCAC |
| SEQ ID | GI 197116369 NG_008383 Homo sapiens | GGGGAGGTGTTATGG |
| NO: 3537 | cytochrome P450 family 2 subfamily E member 1 | TCTGTGCTGAACACC |
| | (CYP2E1), RefSeqGene on chromosome 10 [symbol] | TGGTACTCTTGCACA |
| | | CTGGTTGGTCCATAC |
| SEQ ID | GI 334848115 NG_029039 Homo sapiens | AGTGCACACCTGTAT |
| NO: 3538 | major histocompatibility complex, class 1, G | CCTAACATTTGAGGA |
| | (HLA-G), RefSeqGene on chromosome 6 | GCTGACACAGGAGGA |
| | | ACACTTGCAGCCAGG |
| SEQ ID | GI 308210781 NG_027799 Homo sapiens | AGCACATCACTTTTG |
| NO: 3539 | interferon related developmental regulator 1 | CATCTAGTTCCTTGG |
| | (IFRD1), RefSeqGene on chromosome 7 | CTTACCAGCCACCTG |
| | | GAGGCTGGGAATAGT |
| SEQ ID | GI 429535870 NG_033021 Homo sapiens | AGGATGCCCAAATTC |
| NO: 3540 | interleukin 17A | TGAGGACAAGAACTT |
| | (IL17A), RefSeqGene on chromosome 6 | CCCCCGGACTGTGAT |
| | | GGTCAACCTGAACAT |
| SEQ ID | GI 683524074 NG_034246 Homo sapiens | AAAGGAAATGTGGGG |
| NO: 3541 | lipase E, hormone sensitive type | AAGCAGATCCATGTG |
| | (LIPE), RefSeqGene on chromosome 19 | GCTACAGGTGCCAGA |
| | | CCCCTAGAAATGTGA |
| SEQ ID | GI 237858777 NG_012128 Homo sapiens | AGACCCAGCTCTCTG |
| NO: 3542 | solute carrier family 11 member 1 | CAGCTTAAATAGGGA |
| | (SLC11A1), RefSeqGene on chromosome 2 | GGGAGGTGAGCAGAC |
| | | AGGAATGAATACCCA |
| SEQ ID | GI 1216866445 NM_005302 Homo sapiens | GGGCATACCTTGCTG |
| NO: 3543 | G protein-coupled receptor 37 | GGACTCAACTTGGCT |
| | (GPR37), mRNA | GAGAAATGCACAAGA |
| | | TGCCAAAGGAGGAAG |
| SEQ ID | GI 219802033 NM_001143835 Homo sapiens | CCAAAGCAGTCTCCA |
| NO: 3544 | nuclear factor related to | CTGTTGTTGTGACTA |
| | kappaB binding protein | CAGCTCCGTCTCCTA |
| | (NFRKB), transcript variant 1, mRNA | AACAGGCACCTGAGC |
| SEQ ID | GI 30039658 AY273801 Homo sapiens | ATTGGCAGGCAACAT |
| NO: 3545 | breast cancer 1, early onset | GAATCCAGACTTCTA |
| | (BRCA1) gene, complete cds | GGCTGTCTTGCGGGC |
| | | TCTTTTTTGCCAGTC |
| SEQ ID | GI 3041866 U94788 Human p53 | GCACAGACCCTCTCA |
| NO: 3546 | (TP53) gene, complete cds | CTCATGTGATGTCAT |
| | | CTCTCCTCCCTGCTT |
| | | CTGTCTCCTACAGCC |
| SEQ ID | GI 1024846763 NG_046978 Homo sapiens | AGCATCTTTATATGC |
| NO: 3547 | Charcot-Leyden crystal galectin | ACAGTGAGCAGGTGT |
| | (CLC), RefSeqGene on chromosome 19 | GTGTGACTCAGTGAG |
| | | TGGCACGGCTCTGTG |
| SEQ ID | GI 335057570 NG_029067 Homo sapiens | GACCCCATGGACTGG |
| NO: 3548 | fibroblast growth factor 2 | GTTTGGTCTATGAGC |
| | (FGF2), RefSeqGene on chromosome 4 | AGCAGTATTCACTGG |
| | | AAGTCAACCTGGCTT |
| SEQ ID | GI 398303864 NG_032876 Homo sapiens | GCAGTCATGCTGGAT |
| NO: 3549 | major histocompatibility | AGGGAAGAAGGTGAG |
| | complex, class II, DQ alpha 1 | CTGGAAGAGGAACAG |
| | (HLA-DQA1), RefSeqGene on chromos [symbol] | ACAACTTGGACAGCC |
| SEQ ID | GI 1147592400 NG_029037 Homo sapiens | GTCACCTGCCTGGCC |
| NO: 3550 | reticulon 4 | CTAAGGACATTTGAG |
| | (RTN4), RefSeqGene on chromosome 2 | ACCTCCCTGTGTTCT |
| | | TCTCTTTGTCTTGGG |
| SEQ ID | GI 194294528 NG_008241 Homo sapiens | GGGGCTTTTCCCACA |
| NO: 3551 | succinate-CoA ligase | TGGACACCTGGTGAA |
| | ADP-forming beta subunit | GAGTATTCTAGGCAG |
| | (SUCLA2), RefSeqGene on chromosome 13 | AGGGTGCATTAGAGC |
| SEQ ID | GI 1024336758 NG_046923 Homo sapiens | GGCTACACTACAAAA |
| NO: 3552 | zinc fingers and homeoboxes 3 | GGCAAGAATGCCCAT |
| | (ZHX3), RefSeqGene on chromosome 20 | GTAAGGAGCCCAGCA |
| | | GTCTGGACAGATCCT |
| SEQ ID | GI 401709925 NM_015953 Homo sapiens | TGGCTACCTGTATGA |
| NO: 3553 | nitric oxide synthase interacting protein | GCGTGAGGCCATCCT |
| | (NOSIP), transcript variant 1, mRNA | GGAGTACATTCTGCA |
| | | CCAGAAGAAGGAGAT |
| SEQ ID | GI 189458805 NG_007981 Homo sapiens | ATTGGAGGAGGGAGG |
| NO: 3554 | ATP binding cassette subfamily A member 1 | GAGGGAAGGAAGCTG |
| | (ABCA1), RefSeqGene on chromosome 9 | TGTTGGTTTTCACAC |
| | | AGGGATTGATGGAAT |
| SEQ ID | GI 336391122 NG_029141 Homo sapiens | GGCCCCAGCCTAAAT |
| NO: 3555 | interferon gamma inducible protein 16 | CCCTTCCTAGAGCAA |
| | (IFI16), RefSeqGene on chromosome 1 | GTCCTCACTGGCAAC |
| | | CACGTTTCAGATTAG |
| SEQ ID | GI 24430203 NM_153480 Homo sapiens | AGCTCTGCTTCAAGT |
| NO: 3556 | interleukin 17 receptor E | TCTCTTTTGGAAACA |
| | (IL17RE), transcript variant 1, mRNA | GCAGCCATGTTGAAT |
| | | GCCCCCACCAGACTG |
| SEQ ID | GI 11342663 NM_002463 Homo sapiens | CGCTGCTTTCCTCGC |
| NO: 3557 | MX dynamin like GTPase 2 | CAAGGACTTCAACTT |
| | (MX2), mRNA | TCTCACTTTGAACAA |
| | | TCAGCCACCACCAGG |
| SEQ ID | GI 3403164 AFO27148 Homo sapiens | ACTCTGGAGTCTGCA |
| NO: 3558 | myogenic determining factor 3 | CTCAACTGGTCTGCA |
| | (MYOD1) gene, complete cds | AACCGACTCTCGGAG |
| | | ACTTCAGGTGAGATG |
| SEQ ID | GI 589269250 NG_007475 Homo sapiens | CTGCAGCTGCCCCAG |
| NO: 3559 | aquaporin 1 | GACTGCATGTATGTC |
| | (Colton blood group) | CATGGCAGAAGTAGG |
| | (AQP1), RefSeqGene on chromosome 7 | CAAGAGTTCTAGAAG |
| SEQ ID | GI 225543321 NG_011651 Homo sapiens | TGTGTAATAAGCAGA |
| NO: 3560 | NLRfamily pyrin domain containing 1 | TGCAAAGCCCTGGAC |
| | (NLRP1), RefSeqGene on chromosome 17 | CCCAGACCCACAGAA |
| | | ACATGCCTACCTCAT |
| SEQ ID | GI 51479142 NM_001003703 Homo sapiens | AAGGAAGCACAACGC |
| NO: 3561 | ATP synthase, H+ transporting, | CCCAATCCGCTTTGT |
| | mitochondrial Fo complex subunit F6 | TTTACACAGTCTCAC |
| | (ATP5J), transcript [symbol] | TTTCCCTGCACCTTT |
| SEQ ID | GI 343962631 NM_002037 Homo sapiens | CCCTGCAGTTGATCA |
| NO: 3562 | FYN proto-oncogene, Src family | AGAGACTGGGAAATG |
| | tyrosine kinase | GGCAGTTTGGGGAAG |
| | (FYN), transcript variant 1, mRNA | TATGGATGGGTACCT |
| SEQ ID | GI 117606573 EF064761 Homo sapiens | CTAGTTTGCAGGGGA |
| NO: 3563 | chemokine | AAGTCCTTCACAGGG |
| | (C-C motif) ligand 28 | AAGCATCTCACTGGA |
| | (CCL28) gene, complete cds | CATGCTGGGCTACAA |
| SEQ ID | GI 507834151 NG_008376 Homo sapiens | iTGAACAAGTGGACAA |
| NO: 3564 | cytochrome P450 family 2 | GTGTCTGGGAGTGGA |
| | subfamily D member 6 | CAAGAGATCTGTGCA |
| | (CYP2D6), RefSeqGene on chromosome 2: | CCATCAGGTGTGTGC |
| SEQ ID | GI 306482672 NG_027674 Homo sapiens | CAGAATCGGGTCCAC |
| NO: 3565 | DNA damage inducible transcript 3 | TGGGCAAAATACATG |
| | (DDIT3), RefSeqGene on chromosome 12 | ACTTCTGGCAGGCTG |
| | | AGGGCTTACCAACTA |
| SEQ ID | GI 237858805 NG_012139 Homo sapiens | CGTGAAAGTCAGACT |
| NO: 3566 | uncoupling protein 1 | TCAAGCACAGAGCCA |
| | (UCP1), RefSeqGene on chromosome 4 | TCTCCACGGAATCAA |
| | | ACCTCGCTACACGGG |
| SEQ ID | GI 215277008 NM_000851 Homo sapiens | TCAAAGTCCCCCTCC |
| NO: 3567 | glutathione S-transferase mu 5 | TAACGTCTTCCTTTC |
| | (GSTM5), mRNA | CCTGCACTAACGCCA |
| | | ACCTGACTGCTTTTC |
| SEQ ID | GI 4755084 AFO17178 Homo sapiens | AAGTAGCTTCTGAGA |
| NO: 3568 | pro alpha 1(1) collagen | GGGTGAGGGGTACAC |
| | (COL1A1) gene, complete cds | AGAGAGGGATTATGG |
| | | GAGAGGTCTCTGCCT |
| SEQ ID | GI 27357272 AY195619 Homo sapiens | GGACCCTCCATGTTT |
| NO: 3569 | interleukin 10 receptor, alpha | GCTGGGTATTAGCCA |
| | (IL10RA) gene, complete cds | AGCTGGTCCTGGGAG |
| | | AATGCAGATACTGTC |
| SEQ ID | GI 66841734 DQ073384 Homo sapiens | CTGCTTGGACACTTG |
| NO: 3570 | phosphatase and tensin homolog | GTGTAATAGCTGCTG |
| | (mutated in multiple advanced cancers 1) | CCACTATGCCTCGAA |
| | (PTEN) gene, comp [symbol] | ACTGGTGACTCTGCT |
| SEQ ID | GI 307611940 NG_027759 Homo sapiens | TGTATGCCACACAGA |
| NO: 3571 | caspase recruitment domain family member 11 | ACACTAGTTCGTGAG |
| | (CARD11), RefSeqGene on chromosome 7 | TTGCTCCATGAACCA |
| | | AGGGTGCCATGGTCA |
| SEQ ID | GI 1172222469 NG_053051 Homo sapiens | AACATTTGGGGCCGG |
| NO: 3572 | heat shock protein family H | CTAATCAATTGTGGG |
| | (HspllO) member 1 | GGCTGTCCTGTGCCT |
| | (HSPH1), RefSeqGene on chromosome 13 | TGTAGGATGTTTAAC |
| SEQ ID | GI 37675281 NM_020664 Homo sapiens | CCAAACGGTGTCAAA |
| NO: 3573 | 2,4-dienoyl-CoA reductase 2 | GGGCTGCCGGATTTC |
| | (DECR2), mRNA | GCATCCTTCTCTGCT |
| | | AAGCTCTAGGAATCT |
| SEQ ID | GI 40807361 NM_004637 Homo sapiens | GCTCCCTTCCTAGGA |
| NO: 3574 | RAB7A, member RAS oncogene family | TCTGCCCCTGCATTG |
| | (RAB7A), mRNA | TAGCTTGCTTAACGG |
| | | AGCACTTCTCCTTTT |
| SEQ ID | GI 10280531 AB038161 Homo sapiens | GAGGCTGTTAGACGC |
| NO: 3575 | ABCG1 gene for ABC transporter | TGTGGAGGAAAGGTG |
| | (ATP-binding cassette, sub-family G | GTCAACTGTATGCAT |
| | (WHITE), member 1), com | TGCTTTGCTGTTGGC |
| SEQ ID | GI 41324131 AY527817 Homo sapiens | TGCTAGTGTGTTTAG |
| NO: 3576 | arachidonate 12-lipoxygenase | AGGGCTGAGGATGTC |
| | (ALOX12) gene, complete cds | CCAAAGGCGAGGTGT |
| | | CTTCCCAGATACTGT |
| SEQ ID | GI 343962600 NG_029791 Homo sapiens | AGGTTCTGTTTACTT |
| NO: 3577 | TNF receptor superfamily member 1B | GTGACCCTGAGAAGG |
| | (TNFRSF1B), RefSeqGene on chromosome 1 | GGATGCCCTATGGGG |
| | | GCTCTTCTGAGATCA |
| SEQ ID | GI 498917180 NM_032375 Homo sapiens | ACTATGGGCTGATTA |
| NO: 3578 | AKT1 substrate 1 | AGCCCGGTTGGCTGC |
| | (AKT1S1), transcript variant 1, mRNA | CATGTTGTTAACGAG |
| | | CACCGATTTCCTCTA |
| SEQ ID | GI 166064064 NM_002831 Homo sapiens | CAGGGTGGGGGATCA |
| NO: 3579 | protein tyrosine phosphatase, | GGTGACCCATATTCG |
| | non-receptor type 6 | GATCCAGAACTCAGG |
| | (PTPN6), transcript variant 1, mRNA | GGATTTCTATGACCT |
| SEQ ID | GI 338797748 NM_006743 Homo sapiens | GTTGGGCAGAGATGC |
| NO: 3580 | RNA binding motif | ACTGAACTCATCTTT |
| | (RNP1, RRM) protein 3 | GTGTATGAGGGCCAT |
| | (RBM3), mRNA | GAGAGAGGCCTGGTT |
| SEQ ID | GI 124001557 NM_152586 Homo sapiens | CTGGGCAGCAGTACT |
| NO: 3581 | ubiquitin specific peptidase 54 | GACTTGGGGACTTCC |
| | (USP54), transcript variant 1, mRNA | TTGCCTTTGGATTCC |
| | | TGGGTGAATATCACA |
| SEQ ID | GI 117606557 EF064753 Homo sapiens | ACTGCTGACAAAAGA |
| NO: 3582 | CD40 ligand | GGGGGCTTCTGGTCT |
| | (TNF superfamily, member 5, | GGTTGGTTATTTGTG |
| | hyper-IgM syndrome) | TTTGGCCTGATGTGC |
| | (CD40LG) gene, complete cds [symbol] | |
| SEQ ID | GI 117606537 EF064743 Homo sapiens | TTTCCCTCATAGCAC |
| NO: 3583 | chemokine | CTTCAAGCCTTTCCC |
| | (C-X-C motif) ligand 13 | CAGTATAGCTCCAGG |
| | (B-cell chemoattractant) | GCCTGGGAAAACAAA |
| | (CXCL13) gene, complete cds | |
| SEQ ID | GI 224809353 NG_011513 Homo sapiens | CAGACAGAGCTACAT |
| NO: 3584 | ATP binding cassette subfamily B member 1 | GGAGTCTTGGCAGAG |
| | (ABCB1), RefSeqGene on chromosome 7 | ATGCTGCTCAGACAT |
| | | GCATGGAAACCCTGG |
| SEQ ID | GI 224451013 NG_009888 Homo sapiens | TATTCTCCACATGCG |
| NO: 3585 | acetyl-CoA acetyltransferase 1 | TAAGCAAGGGAGTAC |
| | (ACAT1), RefSeqGene on chromosome 11 | GCACCACAGATTGTC |
| | | AGGCAAAGCCTCAGG |
| SEQ ID | GI 324711016 NG_012837 Homo sapiens | AGTCTCTTCTTGCCT |
| NO: 3586 | GC, vitamin D binding protein | CATTTGCACCCCCCG |
| | (GC), RefSeqGene on chromosome 4 | GCAGCTCAAACCTAC |
| | | TTGTTTCAACAATGC |
| SEQ ID | GI 262331561 NG_013364 Homo sapiens | ACCCATTTTAAAGGT |
| NO: 3587 | transforming growth factor beta 1 | GAGGAAACAAGCCCA |
| | (TGFB1), RefSeqGene on chromosome 19 | GAGAGGTTAAGGGAG |
| | | GAGTTCCTGCCCACC |
| SEQ ID | GI 256220780 NG_012741 Homo sapiens | CCCTGGAGCTTCTAC |
| NO: 3588 | very low density lipoprotein receptor | ACATACCCCTTCTGT |
| | (VLDLR), RefSeqGene on chromosome 9 | CCACTGGCACATGGT |
| | | GCTGTTCCACAATAC |
| SEQ ID | GI 1057867548 NM_020661 Homo sapiens | ATTCAGTTCGTCTCT |
| NO: 3589 | activation induced cytidine deaminase | CCAGACAGCTTCGGC |
| | (AICDA), transcript variant 1, mRNA | GCATCCTTTTGCCCC |
| | | TGTATGAGGTTGATG |
| SEQ ID | GI 28144902 NM_016584 Homo sapiens | TGAGGGAACCAAACC |
| NO: 3590 | interleukin 23 subunit alpha | AGAGACGCGCTGAAC |
| | (IL23A), mRNA | AGAGAGAATCAGGCT |
| | | CAAAGCAAGTGGAAG |
| SEQ ID | GI 2828023 AF036905 Homo sapiens | GAGAACGAGGAACCA |
| NO: 3591 | linker for activation of T cells | GCCTGTGAGGATGCA |
| | (LAT) mRNA, complete cds | GATGAGGATGAGGAC |
| | | GACTATCACAACCCA |
| SEQ ID | GI 48762927 NM_002881 Homo sapiens | AGGGCTGGGTTGGTA |
| NO: 3592 | RAS like proto-oncogene B | AAGAGAAGGCTATGG |
| | (RALB), mRNA | TTGACTTCTTGCTTG |
| | | TGCTTCCCACTCTCC |
| SEQ ID | GI 209447071 NM_005639 Homo sapiens | TGTGTGTGTGTGTGT |
| NO: 3593 | synaptotagmin 1 | GTGTGTGTGTGTGTG |
| | (SYT1), transcript variant 1, mRNA | CACATTTGTTTGGGG |
| | | ATGGGGGAGAAGAAG |
| SEQ ID | GI 937628170 KP290484 Homo sapiens | TGATGCCTTTGTCAT |
| NO: 3594 | OR10J3 | CTCCCACTTCTTCTG |
| | (OR10J3) gene, complete cds | TGATGTGAGACACCT |
| | | GCTGAAGCTGGCCTG |
| SEQ ID | GI 237820683 NG_012068 Homo sapiens | AACACTGAGGCTGAG |
| NO: 3595 | forkhead box 11 | AAAAGCTAGATCACT |
| | (FOXIl), RefSeqGene on chromosome 5 | TGTCAGGGCCTGCAG |
| | | TGATCCCAAGGTTGG |
| SEQ ID | GI 293629222 NG_017001 Homo sapiens | TCTATGCCCCAAGGC |
| NO: 3596 | hepatitis A virus cellular receptor 1 | CATCTGCCACACTGC |
| | (HAVCR1), RefSeqGene on chromosome 5 | CTGTAGTGGTAGGAG |
| | | ACAACTACCTAGTTC |
| SEQ ID | GI 225007577 NG_011607 Homo sapiens | CAACCCAGGTGCCCC |
| NO: 3597 | MER proto-oncogene, tyrosine kinase | CTTGGTCATGAATGA |
| | (MERTK), RefSeqGene on chromosome 2 | TGGACTGCTCTCTCC |
| | | TTAAGGGTTGAGATG |
| SEQ ID | GI 285026427 NG_016458 Homo sapiens | CCAGGATTCACCCCT |
| NO: 3598 | protein kinase AMP-activated | ATTGGACTAATTCTG |
| | non-catalytic subunit gamma 3 | TCAATGCCCACTTCA |
| | (PRKAG3), RefSeqGene on chr [symbol] | CTGTGGCCAGAGGCA |
| SEQ ID | GI 94538326 NM_002038 Homo sapiens | TCAACCTCCCAAGTA |
| NO: 3599 | interferon alpha inducible protein 6 | GGATTACAAGCATGC |
| | (IFI6), transcript variant 1, mRNA | GCCGACGATGCCCAG |
| | | AATCCAGAACTTTGT |
| SEQ ID | GI 386781923 NM_002371 Homo sapiens | TGAAAACATTGCTGC |
| NO: 3600 | mal, T-cell differentiation protein | CGTGGTGTTCTCCTA |
| | (MAL), transcript variant a, mRNA | CATAGCCACTCTGCT |
| | | CTACGTGGTCCATGC |
| SEQ ID | GI 18765742 NM_003098 Homo sapiens | AAGCTGCAGATGTCT |
| NO: 3601 | syntrophin alpha 1 | TCAGATGACGGTGCC |
| | (SNTA1), mRNA | AGTCTCCTTTTCCTG |
| | | GATTTTGGAGGTGCT |
| SEQ ID | GI 1015637181 AH000040 Homo sapiens | CGTATACCTGCTCAC |
| NO: 3602 | hematopoietic stem cell antigen | TCCTGACGCTTCACT |
| | (CD34) gene, complete cds | CACACACAGCACAGG |
| | | ATCTGGTGAGGCTAT |
| SEQ ID | GI 6537133 AF102137 Homo sapiens | TACAGCAGGAAACTG |
| NO: 3603 | PERI gene, complete cds | CACCAGCTAGACTCC |
| | | ATTCTGGGACCATCT |
| | | CCAGGAGTCCATGAG |
| SEQ ID | GI 189571639 NG_007991 Homo sapiens | GTGACACATATAGCC |
| NO: 3604 | acyl-CoA dehydrogenase, | ACGGCTTCCTTTGCC |
| | C-2 to C-3 short chain | TGGCCTGAATTACAA |
| | (ACADS), RefSeqGene on chromosome 12 | GGGGAACGCTACTGA |
| SEQ ID | GI 209447054 NG_008716 Homo sapiens | GATGTGGTGACAGGT |
| NO: 3605 | aminolevulinate dehydratase | GCTCTGAAGGAGGTC |
| | (ALAD), RefSeqGene on chromosome 9 | ACCAGGACCTGACCT |
| | | CTTTGAAGGATCAGA |
| SEQ ID | GI 190341054 NG_008010 Homo sapiens | CTTCCAGCCACTGCA |
| NO: 3606 | aldolase, fructose-bisphosphate A | GGCTCTGCCCCTTCA |
| | (ALDOA), RefSeqGene on chromosome 16 | CCTAACAGCATAAGA |
| | | TAGGGCTAACAGTTG |
| SEQ ID | GI 301500676 NG_023437 Homo sapiens | ACATTGAATTTTCAA |
| NO: 3607 | apolipoprotein L4 | CCTGGCTATCCTGGA |
| | (APOL4), RefSeqGene on chromosome 22 | GATGTCCCCAGTCCA |
| | | TACACCCGATGGGGC |
| SEQ ID | GI 237858770 NG_012124 Homo sapiens | TCTTTTTAAGCTGGG |
| NO: 3608 | selectin E | ACACCCATCTTCTCC |
| | (SELE), RefSeqGene on chromosome 1 | TGCCTTCCAACACTG |
| | | GCACTCTTGGTTCCA |
| SEQ ID | GI 229577384 NG_011990 Homo sapiens | ATGCCCTCAAGGTTC |
| NO: 3609 | SMAD family member 3 | CTCAAGGGTCAGCTG |
| | (SMAD3), RefSeqGene on chromosome 15 | TCACAGTTCTCCAAA |
| | | GTGAGTTTTCAGGCA |
| SEQ ID | GI 190194362 NM_006426 Homo sapiens | CCTCTGGGAAGATGG |
| NO: 3610 | dihydropyrimidinase like 4 | ACGAGAATGAGTTCG |
| | (DPYSL4), mRNA | TCGCGGTGACCAGTA |
| | | CAAATGCTGCCAAAA |
| SEQ ID | GI 48526521AY644470 Homo sapiens | ACTCATCTGAAGGCT |
| NO: 3611 | interferon gamma receptor 2 | TAAACTACACTGGCA |
| | (interferon gamma transducer l) | GTCAGGACTCCGCTG |
| | (IFNGR2) gene, complete cds | TCTAACCACACGGGT |
| SEQ ID | GI 6537133 AF102137 Homo sapiens | GGTGCCCAAGCTAGA |
| NO: 3612 | PERI gene, complete cds | GGAGGGCAGAGAAGA |
| | | ATGCCTTGCTTGCTC |
| | | TCAGTCATGTTACTG |
| SEQ ID | GI 336176054 NG_029124 Homo sapiens | ACAAAAGTCGGCAGA |
| NO: 3613 | caspase 1 | GGTGCCATGCTCAGA |
| | (CASP1), RefSeqGene on chromosome 11 | ACCCAAGGATAGAGT |
| | | GGGGTGAAAGAAACA |
| SEQ ID | GI 209447084 NG_008733 Homo sapiens | GGCCCTCACCAATAC |
| NO: 3614 | copper chaperone for superoxide dismutase | TCTGTGAGGCACATA |
| | (CCS), RefSeqGene on chromosome 11 | CACACAGGCACAACC |
| | | TCCAGATGAGGAAAC |
| SEQ ID | GI 238018083 NG_012204 Homo sapiens | CCTGGGCCTCTCTGT |
| NO: 3615 | peroxisome proliferator | GTTTGGTTCCAAGCA |
| | activated receptor alpha | CTTCCCACCTCAAAC |
| | (PPARA), RefSeqGene on chromosome 22 | TCCCATTTTCAAACC |
| SEQ ID | GI 42794766 NM_203351 Homo sapiens | CAAGTTCGAGCACAA |
| NO: 3616 | mitogen-activated protein kinase kinase kinase 3 | CGGGGAGAGGCGAAT |
| | (MAP3K3), transcript variant 1, mRNA | TATAGCGTTCAGCCG |
| | | GCCTGTGAAATATGA |
| SEQ ID | GI 37595566 NM_007144 Homo sapiens | GTGCCTGCATTCCTT |
| NO: 3617 | polycomb group ring finger 2 | CTGCAAAACCTGCAT |
| | (PCGF2), mRNA | CGTGCGCTACCTGGA |
| | | GACCAACAAATACTG |
| SEQ ID | GI 166706752 EU332835 Homo sapiens | GGGATGCCTGTAGTT |
| NO: 3618 | v-akt murine thymoma viral oncogene homolog l | GGTGATGTGGGAACT |
| | (AKT1) gene, complete cds | GGGTTTTGAGGGATG |
| | | CCTAGGAGTTCATCC |
| SEQ ID | GI 29825390 AY242132 Homo sapiens | CCCAATGACTACCTG |
| NO: 3619 | cannabinoid receptor 2 | CTGAGCTGGCTCCTG |
| | (CNR2) gene, complete cds | TTCATCGCCTTCCTC |
| | | TTTTCCGGAATCATC |
| SEQ ID | GI 290543602 GU130197 Homo sapiens | GATGTTCTCTTGAGG |
| NO: 3620 | isolate MIA060410 Duffy | TGTGGGGTCACGGCC |
| | antigen/chemokine receptor | ATCTCAAGGGAGAAG |
| | (DARC) gene, complete cds | AGAAGAGGAACCAGA |
| SEQ ID | GI 62632868 AY995114 Homo sapiens | GGGGATTACCCACCC |
| NO: 3621 | tumor necrosis factor receptor | ACTATAATGGAGCGG |
| | 1-associated death domain protein | GTAGTCAAGACCGAG |
| | (TRADD) gene, complete cds [symbol] | GAGTGTCTGTTGGAC |
| SEQ ID | GI 336176054 NG_029124 Homo sapiens | CCCTGAAGAAACTCT |
| NO: 3622 | caspase 1 | AGGATGCATGGTGTG |
| | (CASP1), RefSeqGene on chromosome 11 | CACAGTGAAGTCCTG |
| | | GCATGGTGCAGAGAA |
| SEQ ID | GI 761631399 NG_042041 Homo sapiens | ATACAGGCAGCTCAT |
| NO: 3623 | integrin subunit beta 6 | TCTATTCTCAGCCAC |
| | (ITGB6), RefSeqGene on chromosome 2 | AGCCTCAGAAAGGAG |
| | | GGACCCAGACCAGAA |
| SEQ ID | GI 206725550 NM_004958 Homo sapiens | GTTGATCCTTAACGA |
| NO: 3624 | mechanistic target of rapamycin | GCTGGTCCGAATCAG |
| | (MTOR), mRNA | CAGCATGGAGGGAGA |
| | | GCGTCTGAGAGAAGA |
| SEQ ID | GI 54781368 AY791292 Homo sapiens | GCCCCTGGTCTATAC |
| NO: 3625 | cyclin GI | CTGGTACTTGGAGGT |
| | (CCNG1) gene, complete cds | AGGATTCAAAGCCAG |
| | | GCAGTTCTGACTCCA |
| SEQ ID | GI 306816 M96233 Human glutathione | CCCAGGGCACTTTGG |
| NO: 3626 | transferase class mu number 4 | AAGAGGCAGAGAACT |
| | (GSTM4) gene, complete cds | TTAGGAGCATGGATG |
| | | CAGCTGGCAATAGTA |
| SEQ ID | GI 197100280 NG_008374 Homo sapiens | GAGTCAGACAGGTGA |
| NO: 3627 | cytochrome P450 family 11 | AAATGGAGAATTGCC |
| | subfamily B member 2 | ACTCACCAAGGGCAG |
| | (CYP11B2), RefSeqGene on chromosome [symbol] | GCTTCTTGAGGGCTA |
| SEQ ID | GI 209447063 NG_008722 Homo sapiens | CCCATTAGGGGTGCT |
| NO: 3628 | lysyl oxidase | GCTTTGGTCTGCCAT |
| | (LOX), RefSeqGene on chromosome 5 | TCTGACACTATGTCT |
| | | GGCATAGCAGAGGTC |
| SEQ ID | GI 341572553 NG_029518 Homo sapiens | GTGCCTATGTGATTG |
| NO: 3629 | sestrin 1 | GAAATCCATTACCCA |
| | (SESN1), RefSeqGene on chromosome 6 | GTGGAGCAACGCACA |
| | | CAACTGGTGCATGCA |
| SEQ ID | GI 124001557 NM_152586 Homo sapiens | TGGGCTGGTATGGGA |
| NO: 3630 | ubiquitin specific peptidase 54 | CTCAGACCACTCAGA |
| | (USP54), transcript variant 1, mRNA | CTTAGCAGAAGATGT |
| | | TATCCACAGCCTGGG |
| SEQ ID | GI 24371240 NM_024630 Homo sapiens | GGTCTCGTGTTGAGA |
| NO: 3631 | zinc finger DHHC-type containing 14 | TATTGTGTGCCACAA |
| | (ZDHHC14), transcript variant 1, mRNA | CCCCCACAGTCTTCA |
| | | CCTCCGTGTGTGATG |
| SEQ ID | GI 18653445 AF479264 Homo sapiens | TGTGTGCAGTAGCAG |
| NO: 3632 | ligase IV, DNA, ATP-dependent | AGAAGCCCCCTCCTG |
| | (LIG4) gene, complete cds | GTGAGAAGCCATCTG |
| | | TGTTTCATACTCTCT |
| SEQ ID | GI 239787264 NG_012303 Homo sapiens | GCGCGTCTTTCTCAT |
| NO: 3633 | colony stimulating factor 1 receptor | AGTGATGTTAGCAGC |
| | (CSF1R), RefSeqGene on chromosome 5 | ATCCGCAGGCAAGGA |
| | | GAAACATGCAATGCC |
| SEQ ID | GI 197313719 NG_008421 Homo sapiens | TAGTTCAATCCCCAA |
| NO: 3634 | cytochrome P450 family 3 subfamily A member 4 | GCCCCTCCAAAGTGT |
| | (CYP3A4), RefSeqGene on chromosome 7 | CTTCTCCTCCTAGTC |
| | | CAGGGCCCATTTACT |
| SEQ ID | GI 63054827 NM_001915 Homo sapiens | AAGTCAGGATGGAGT |
| NO: 3635 | cytochrome b561 | CAGAACAACTACAGT |
| | (CYB561), transcript variant 1, mRNA | CATCCCCCGTGGTGT |
| | | CTGCACATCACTCCA |
| SEQ ID | GI 13430063 AF253322 Homo sapiens | TGTCAGGGAAAGGGG |
| NO: 3636 | cytochrome P450 | AAACCTTTCCACCAG |
| | (CYP1A1) and cytochrome P450 | AACTGCTTACTGGGC |
| | (CYP1A2) genes, complete cds | ATGAAGAAGAGGGTG |
| SEQ ID | GI 20336662 AF503510 Homo sapiens | ACGAGGGTTTCTGTG |
| NO: 3637 | coagulation factor X | GTGGAACTATTCTGA |
| | (F10) gene, complete cds | GCGAGTTCTACATCC |
| | | TAACGGCAGCCCACT |
| SEQ ID | GI 224809232 NG_011437 Homo sapiens | GGGATAACAGAGAAA |
| NO: 3638 | arachidonate 5-lipoxygenase | TGCTGCAGGCATTGG |
| | (ALOX5), RefSeqGene on chromosome 10 | CCCCAAATCTCAGGC |
| | | CTCCTATTCCCAGTA |
| SEQ ID | GI 352962130 NG_030351 Homo sapiens | GGGGGAAGAGAGGAA |
| NO: 3639 | eukaryotic translation initiation | GTGGGGATCCAGTTA |
| | factor 2 alpha kinase 2 | ACGAGGTACTGCTAT |
| | (EIF2AK2), RefSeqGene on chromoso [symbol] | GGCCTAATGGAAGCA |
| SEQ ID | GI 167614484 NM_006111 Homo sapiens | CTTGTCTGAATTTGC |
| NO: 3640 | acetyl-CoA acyltransferase 2 | TGCCAAGGCTGCCTT |
| | (ACAA2), mRNA | GTCTGCTGGCAAAGT |
| | | CTCACCTGAAACAGT |
| SEQ ID | GI 116734711 NM_000020 Homo sapiens | CTTGGGCTTTCTGTC |
| NO: 3641 | activin A receptor like type 1 | TCCTCAACAAGAGTG |
| | (ACVRL1), transcript variant 1, mRNA | CAGCTTGCTGAATGT |
| | | CAGCTGCCTGAGAGA |
| SEQ ID | GI 41872630 NM_004104 Homo sapiens | GAGCACAGACGAGAG |
| NO: 3642 | fatty acid synthase | CACCTTTGATGACAT |
| | (FASN), mRNA | CGTCCATTCGTTTGT |
| | | GAGCCTGACTGCCAT |
| SEQ ID | GI 49574531 NM_019884 Homo sapiens | GCCTCACCCCTGTGG |
| NO: 3643 | glycogen synthase kinase 3 alpha | TGGGCTTTTTAAGAG |
| | (GSK3A), mRNA | GATTTTAACTGGTTG |
| | | TGGGGAGGGAAGAGA |
| SEQ ID | GI 386781923 NM_002371 Homo sapiens | TACCTGTTGGTATTG |
| NO: 3644 | mal, T-cell differentiation protein | TCCACAAGCTCTCCC |
| | (MAL), transcript variant a, mRNA | GAGCGCCCCATCTTG |
| | | TGCCATGTTTTAAGT |
| SEQ ID | GI 218083972 NM_134431 Homo sapiens | TCGGTGCATGGTGGT |
| NO: 3645 | solute carrier organic anion | TTGGCTTTCTGATTT |
| | transporter family member 1A2 | GTGCAGGAGTTAACG |
| | (SLCO1A2), transcript variant 1, [symbol] | TGCTCACTGCCATTC |
| SEQ ID | GI 1015809708 NM_003807 Homo sapiens | CCAGCAATTTCCATC |
| NO: 3646 | TNF superfamily member 14 | ACCTCTCAATGCTTC |
| | (TNFSF14), transcript variant 1, mRNA | AGTTTCCCCATCGGC |
| | | AAGATGGTTGTGGGG |
| SEQ ID | GI 29501801 AY260471 Homo sapiens | ACATGTGGTCTCGGC |
| NO: 3647 | BCL2-antagonist/killer 1 | CCCCTATTTTTCTCT |
| | (BAK1) gene, complete cds | TTCGGGGTCCCAGAC |
| | | TTGTATTTTGGGCAT |
| SEQ ID | GI 299782537 NG_023178 Homo sapiens | CGGAGGCTTTCACTG |
| NO: 3648 | CD14 molecule | TGTCACTCCCCGACA |
| | (CD14), RefSeqGene on chromosome 5 | TAATGGGAAATTTGG |
| | | GCCTAGGGAACTGAC |
| SEQ ID | GI 236459493 NG_012010 Homo sapiens | GATTGGCCCATCTCA |
| NO: 3649 | lymphotoxin alpha | AGGAGCATCCTTCGC |
| | (LTA), RefSeqGene on chromosome 6 | ATATCTCACATGCAC |
| | | GTGACACTGAGCCTC |
| SEQ ID | GI 2828023 AF036905 Homo sapiens | GCCTGACAACAGCCT |
| NO: 3650 | linker for activation of T cells | GAGAAATCCCCCCGT |
| | (LAT) mRNA, complete cds | AACTTATTATCACTT |
| | | TGGGGTTCGGCCTGT |
| SEQ ID | GI 1049011196 AH002836 Homo sapiens | GCTGTGGTCCCACTC |
| NO: 3651 | 1g J-chain gene, complete cds | GTATATGGTGGTGAG |
| | ; and IGJ gene, complete sequence | ACCAAAATGGTGGAA |
| | | ACAGCCTTAACCCCA |
| SEQ ID | GI 1036031265 AH007464 Homo sapiens | AAGCTGGAGGTGCCA |
| NO: 3652 | methylenetetrahydrofolate reductase | CAGGAGATCAAGGAC |
| | (MTHFR) gene, complete cds | GTGATTGAGCCAATC |
| | | AAAGACAACGATGCT |
| SEQ ID | GI 32263624 AY325800 Homo sapiens | CTCCTTAAGGGTGCT |
| NO: 3653 | stannin | GTTCCTCAAGGACGC |
| | (SNN) gene, complete cds | AGCAGGTTCGACCCT |
| | | ATTTGGAGGGAAAAC |
| SEQ ID | GI 297747369 NG_021416 Homo sapiens | TCCTTAGCCCTATGA |
| NO: 3654 | growth hormone releasing hormone receptor | CTCCCTGAGCCACTC |
| | (GHRHR), RefSeqGene on chromosome 7 | TCCAGTTTGGTGGTT |
| | | TCCTTTGAAGAGGGT |
| SEQ ID | GI 767172199 NG_042051 Homo sapiens | GTGCTGCAAGGAGCT |
| NO: 3655 | kelch like family member 41 | AAGGCCTTCAGTGTC |
| | (KLHL41), RefSeqGene on chromosome 2 | CCCTTCCTTACCCAG |
| | | GTTTCTCACAGAATG |
| SEQ ID | GI 1063759771 NG_051548 Homo sapiens | AGGCCTGGAGTGGAT |
| NO: 3656 | mitogen-activated protein | TTCCCTCTGTACCCC |
| | kinase-activated protein kinase 3 | TTTATGGGTGAGCAT |
| | (MAPKAPK3), RefSeqGene on [symbol] | GGCTAAGGCTACTAG |
| SEQ ID | GI 618857519 NG_034119 Homo sapiens | CCTGGAGGTTTCTGG |
| NO: 3657 | nuclear receptor subfamily | TTGGGGTGGTTTGGG |
| | 2 group F member 1 | TTTGAGAGGGGACTC |
| | (NR2F1), RefSeqGene on chromosome 5 | CAGGAATCCTTTAAG |
| SEQ ID | GI 194394200 NG_008275 Homo sapiens | CAGGGTTTGAGAGAT |
| NO: 3658 | prokineticin 2 | TGTATCTCCAAGGGT |
| | (PROK2), RefSeqGene on chromosome 3 | TGCAGACCCACCTGT |
| | | CTACAGGGGTCAGGA |
| SEQ ID | GI 493799665 NM_002994 Homo sapiens | CGACATCCTCGAGGC |
| NO: 3659 | C-X-C motif chemokine ligand 5 | CCTAGCATTTCTCCT |
| | (CXCL5), mRNA | TGGATAGGGGACCAG |
| | | AGAGAGCTTGGAATG |
| SEQ ID | GI 187408 M13577 Human myelin basic protein | GGCTCACAAGGGATT |
| NO: 3660 | (MBP) mRNA, complete cds | CAAGGGAGTCGATGC |
| | | CCAGGGCACGCTTTC |
| | | CAAAATTTTTAAGCT |
| SEQ ID | GI 42519913 NM_012448 Homo sapiens | AAAGGCAGTGAGTTT |
| NO: 3661 | signal transducer and | CGTGAAGCCTGGAAG |
| | activator of transcription 5B | TTGGCCATGTGTCTT |
| | (STAT5B), mRNA | AAGAGTGGCTGGACT |
| SEQ ID | GI 24756896 L47234 Homo sapiens | AGAATTCCCGTCTCA |
| NO: 3662 | ERCC2 | GTGGTCAAATTCTCA |
| | (ERCC2) and kinesin light chain | TTCATCGTGGCCCAG |
| | (KLC2) genes, complete cds, | GCAAGCACACTTCCT |
| | complete sequence | |
| SEQ ID | GI 32165625 AY323826 Homo sapiens | AAGCAGGGTCAGGTG |
| NO: 3663 | interleukin 10 receptor, beta | CCTCCTGAGGCAAGT |
| | (IL10RB) gene, complete cds | GTGAATGCAAACTCC |
| | | ACTTACTGTTTAGCT |
| SEQ ID | GI 507834151 NG_008376 Homo sapiens | AGAGCCTGTGACTAT |
| NO: 3664 | cytochrome P450 family 2 subfamily D member 6 | GGAGAGAGGCCACCT |
| | (CYP2D6), RefSeqGene on chromosome 2 [symbol] | GAGGTAGTACCCTCT |
| | | GAGGAACAGGAACAA |
| SEQ ID | GI 1176461486 NG_030337 Homo sapiens | GGAAGAGGACCAAGA |
| NO: 3665 | NLR family CARD domain containing 5 | GGATGGGTTTACTGT |
| | (NLRC5), RefSeqGene on chromosome 16 | GGTGCTGGGGAGGGG |
| | | ATAGTAGCAGAGAAC |
| SEQ ID | GI 292658763 NM_014143 Homo sapiens | AAGACGGGTTGAGAA |
| NO: 3666 | CD274 molecule | TCCCTAATTTGAGGG |
| | (CD274), transcript variant 1, mRNA | TCAGTTCCTGCAGAA |
| | | GTGCCCTTTGCCTCC |
| SEQ ID | GI 664806095 NM_005438 Homo sapiens | CGCAGTGGTTCAGCC |
| NO: 3667 | FOS like 1, AP-1 transcription factor subunit | CGAGAACTTTTCATT |
| | (FOSLl), transcript variant 1, mRNA | CATAAAAAGAAAAGA |
| | | CTCCGCACGGCGCGG |
| SEQ ID | GI 1072314151 NM_000413 Homo sapiens | CTGTGGGTGGCTAAT |
| NO: 3668 | hydroxysteroid 17-beta dehydrogenase 1 | TAAGATAGATCGCGT |
| | (HSD17B1), transcript variant 1, mRNA | TAGCCAGTTTTACCA |
| | | GCGCAGCTAGGCGCG |
| SEQ ID | GI 325495520 HQ692855 Homo sapiens | CAAGGGCTTCTTCAA |
| NO: 3669 | nerve growth factor IB | GCGCACAGTGCAGAA |
| | nuclear receptor variant 1 | AAACGCCAAGTACAT |
| | (NR4A1) mRNA, complete cds | CTGCCTGGCTAACAA |
| SEQ ID | GI 78482607 NM_021129 Homo sapiens | CCACCACCCTGTGAA |
| NO: 3670 | pyrophosphatase | TCTGCCTGCACAGTA |
| | (inorganic) 1 | CCAACAGACGTGGAT |
| | (PPA1), mRNA | AAGTGGTTCCATCAC |
| SEQ ID | GI 21655314 AY124010 Homo sapiens | ACACATGTGAAGGGT |
| NO: 3671 | interleukin 1 receptor, type II | GGCGTTTCAGATCAT |
| | (IL1R2) gene, complete cds | TTCCAGATGAGCAGG |
| | | CTGGGAGAGATGGTG |
| SEQ ID | GI 117606491 EF064720 Homo sapiens | GGTGAGACGCGTTAG |
| NO: 3672 | interleukin 27 | CAGGTGACATCATGG |
| | (IL27) gene, complete cds | AAGATCAAGCAGGGA |
| | | ATGTCCGGGGAAATC |
| SEQ ID | GI 228008299 NG_011964 Homo sapiens | TCTTCTGGAGGAGAC |
| NO: 3673 | arginase 2 | CTCTATAACAATCAG |
| | (ARG2), RefSeqGene on chromosome 14 | CTGAGGCTGCTGAAT |
| | | GAGGGGGAAGGGCAC |
| SEQ ID | GI 221139857 NG_009364 Homo sapiens | CACACATATTTGGGG |
| NO: 3674 | cyclin dependent kinase inhibitor 1A | GGCATTCACACCCAT |
| | (CDKN1A), RefSeqGene on chromosome 6 | GAGGGACACCTCTGG |
| | | GGGGAAAACAAATTG |
| SEQ ID | GI 1033837679 NG_008338 Homo sapiens | GTTGGTTGACTGGCA |
| NO: 3675 | cytochrome P450family 7 subfamily B member 1 | AAACCAGAAGGGCTT |
| | (CYP7B1), RefSeqGene on chromosome 8 [symbol] | CCTCTTGTTTGGCCA |
| | | ATGCTTGAGCTCTCA |
| SEQ ID | GI 953768433 NG_016863 Homo sapiens | GACCACTATTAAACA |
| NO: 3676 | delta like non-canonical Notch ligand 1 | GCCTGGTACAGACGC |
| | (DLK1), RefSeqGene on chromosome 14 | TGGCTTGTGCCACCG |
| | | TAGACAGACCTCGTA |
| SEQ ID | GI 520260356 NM_021913 Homo sapiens | CTGCAGCTTTCCGAC |
| NO: 3677 | AXL receptor tyrosine kinase | ACGGGACAGTACCAG |
| | (AXL), transcript variant 1, mRNA | TGTTTGGTGTTTCTG |
| | | GGACATCAGACCTTC |
| SEQ ID | GI 62530382 NM_004281 Homo sapiens | AACAGAAAGCCATTG |
| NO: 3678 | BCL2 associated athanogene 3 | ATGTCCCAGGTCAAG |
| | (BAG3), mRNA | TCCAGGTCTATGAAC |
| | | TCCAGCCCAGCAACC |
| SEQ ID | GI 347446697 NG_030008 Homo sapiens | ACCAACTATCTTGGT |
| NO: 3679 | colony stimulating factor 1 | AACTGCCTAACTGAC |
| | (CSF1), RefSeqGene on chromosome 1 | CAGCTGTTGCTTGGG |
| | | GAGCCCATTGAGCAA |
| SEQ ID | GI 349732110 NG_030313 Homo sapiens | TGTCTGGCAGGAAAG |
| NO: 3680 | dihydrolipoamide S-succinyltransferase | ATAGAGTGGGCTGGA |
| | (DLST), RefSeqGene on chromosome 14 | CGGTACAGGATCGCG |
| | | TAAAGTGTGGACTTT |
| SEQ ID | GI 345478700 NG_029900 Homo sapiens | CTGCCTCTCCCTTTG |
| NO: 3681 | p21 | TTCTCTGTGCCTCTA |
| | (RAC1) activated kinase 1 | GCCTCCTATCTGCTG |
| | (PAK1), RefSeqGene on chromosome 11 | GAGGTTTGTGCTACA |
| SEQ ID | GI 340523103 NG_029468 Homo sapiens | GATTAGCTCTGTGCT |
| NO: 3682 | SWI/SNF related, matrix associated, actin | GCAGCCAATCCATGT |
| | dependent regulator of chromatin, | TAGCCCCCCCAAGTT |
| | subfamily d, m [symbol] | AGGAAGACCTGGAAA |
| SEQ ID | GI 584458494 NG_033933 Homo sapiens | CAAATGCATCACTAA |
| NO: 3683 | toll like receptor 9 | AACCAAGGCCTTCCA |
| | (TLR9), RefSeqGene on chromosome 3 | GGGCCTAACACAGCT |
| | | GCGCAAGCTTAACCT |
| SEQ ID | GI 197276631 NM_003670 Homo sapiens | ATTGCCCTGCAGAGT |
| NO: 3684 | basic helix-loop-helix family member e40 | GGTTTACAAGCTGGT |
| | (BHLHE40), mRNA | GAGCTGTCAGGGAGA |
| | | AATGTCGAAACAGGT |
| SEQ ID | GI 22165426 NM_002991 Homo sapiens | GCCTGATGACCATAG |
| NO: 3685 | C-C motif chemokine ligand 24 | TAACCAGCCTTCTGT |
| | (CCL24), mRNA | TCCTTGGTGTCTGTG |
| | | CCCACCACATCATCC |
| SEQ ID | GI 345199309 NM_002167 Homo sapiens | CGAGCTCACTCCGGA |
| NO: 3686 | inhibitor of DNA binding 3, HLH protein | ACTTGTCATCTCCAA |
| | (ID3), mRNA | CGACAAAAGGAGCTT |
| | | TTGCCACTGACTCGG |
| SEQ ID | GI 14488407 AF386492 Homo sapiens | CCAACTCTGTTCATG |
| NO: 3687 | serine-cysteine proteinase | ACACTTCCTTCCAGC |
| | inhibitor clade E member 1 | ACCAAATGCTTCCCA |
| | (SERPINE1) gene, complete cds | CCCCTCTACTCCCAC |
| SEQ ID | GI 30526191 AY275469 Homo sapiens | CTTTAACCCGGTGCT |
| NO: 3688 | chemokine C motif receptor 1 | CTATGTCTTCGTGGG |
| | (XCR1) gene, complete cds | GGTCAAGTTCCGCAC |
| | | ACACCTGAAACATGT |
| SEQ ID | GI 209447058 NG_008718 Homo sapiens | TTTGTTGTCCCACTC |
| NO: 3689 | apurinic/apyrimidinic | TCTGTTACCTGCATT |
| | endodeoxyribonuclease 1 | GTGTGACAGCAAGAT |
| | (APEX1), RefSeqGene on chromosome 14 | CCGTTCCAAGGCCCT |
| SEQ ID | GI 341865611 NG_029556 Homo sapiens | GTACTTACGTGCCAT |
| NO: 3690 | growth factor receptor bound protein 2 | TCCCTTTCCCTGAAA |
| | (GRB2), RefSeqGene on chromosome 17 | TGCCCTTACTCTGGC |
| | | TTCTCTGCCTGGCAA |
| SEQ ID | GI 527498317 NG_008179 Homo sapiens | GCAGAGACTCACATG |
| NO: 3691 | general transcription factor IIi | GAGCCAGCTGCTATG |
| | (GTF2I), RefSeqGene on chromosome 7 | CAGAGCTCAAACTTC |
| | | AGCAGAAACCCAGAG |
| SEQ ID | GI 167614499 NM_002286 Homo sapiens | TCTCCTGGTCTCCCT |
| NO: 3692 | lymphocyte activating 3 | TCTTCTAGAACCCCT |
| | (LAG3), mRNA | TCCTCCACCTCCCTC |
| | | TCTGCAGAACTTCTC |
| SEQ ID | GI 7637751 AF250378 Homo sapiens | CCCTGTGGAGGTCAG |
| NO: 3693 | E2F transcription factor 4 | CTATACCAAAAGGGT |
| | (E2F4) gene, complete cds | ACAGAAATTCTGGAG |
| | | TGGGTCCCTGGGTCT |
| SEQ ID | GI 117606677 EF078888 Homo sapiens | TTGGGGAGGGAGTGT |
| NO: 3694 | Kruppel-like factor 2 | TCGCTGGGAAACCTT |
| | (lung) | GAGAGATTCCGTGTC |
| | (KLF2) gene, complete cds | TTAGAATGCTGGAGA |
| SEQ ID | GI 60391402 AB019488 Homo sapiens | TGAGCAGGGATATCT |
| NO: 3695 | NTRK1 gene for TRKA, complete cds | ACAGCACCGACTATT |
| | | ACCGTGTAAGGGTCC |
| | | TTTGTCCCCAACGCC |
| SEQ ID | GI 205361192 NG_008140 Homo sapiens | TATCGGGGGCCACCT |
| NO: 3696 | aminoadipate-semialdehyde synthase | TTTCATACAGGCAGG |
| | (AASS), RefSeqGene on chromosome 7 | TGTCACAGAACTGAC |
| | | TGTGGGACTTGAGTA |
| SEQ ID | GI 223671920 NG_009776 Homo sapiens | AGGACTTACACCAGA |
| NO: 3697 | epoxide hydrolase 1 | GGATCGATAAGTTCC |
| | (EPHX1), RefSeqGene on chromosome 1 | GTTTCACCCCACCTT |
| | | TGGAGGACAGCTGCT |
| SEQ ID | GI 742670592 NG_013347 Homo sapiens | GTGAAGGAATCAGCA |
| NO: 3698 | glutamate-ammonia ligase | TGGGAGATCATCTCT |
| | (GLUL), RefSeqGene on chromosome 1 | GGGTGGCCCGTTTCA |
| | | TCTTGCATCGTGTGT |
| SEQ ID | GI 224809262 NG_011468 Homo sapiens | CACTCCACCCCCGTG |
| NO: 3699 | matrix metallopeptidase 9 | TTCTCATCTCTTCCC |
| | (MMP9), RefSeqGene on chromosome 20 | ACTCCTCCTGTTATT |
| | | TACTCTGCTCCACCC |
| SEQ ID | GI 49574517 NM_003695 Homo sapiens | GGAGGACAGAAGCCC |
| NO: 3700 | lymphocyte antigen 6 family member D | TGTGGATCCCCGGAT |
| | (LY6D), mRNA | TTCACACTCCTTCTG |
| | | TTTTGTTGCCGTTTA |
| SEQ ID | GI 260593637 NG_013005 Homo sapiens | GCTGAACAGCAATCA |
| NO: 3701 | calcitonin receptor | TGGAAACTGATGTGT |
| | (CALCR), RefSeqGene on chromosome 7 | TGTGTCTTAGGGACT |
| | | GAGGGGAGGGGGTGG |
| SEQ ID | GI 429535868 NG_033017 Homo sapiens | TAGCAGGTATCAAGA |
| NO: 3702 | membrane bound transcription factor peptidase, site 1 | CTCATTCCTGCTTGT |
| | (MBTPS1), RefSeqGene on chromoso [symbol] | GGCTGAGCAGTGTCC |
| | | TGTGGTGTGGCTAGA |
| SEQ ID | GI 520260356 NM_021913 Homo sapiens | TGAGTGAAGCGGTCT |
| NO: 3703 | AXL receptor tyrosine kinase | GCATGAAGGAATTTG |
| | (AXL), transcript variant 1, mRNA | ACCATCCCAACGTCA |
| | | TGAGGCTCATCGGTG |
| SEQ ID | GI 806607 M90696 Homo sapiens | CCCGTTACTTGGGAG |
| NO: 3704 | cathepsin S | GCCAAGGCAGGCATT |
| | (CTSS) mRNA, complete cds | ATCAATCTTGAGGCC |
| | | AGGAGTTAAAGAGCA |
| SEQ ID | GI 966751447 NM_002581 Homo sapiens | CAACCAGGAGGAGTT |
| NO: 3705 | pappalysin 1 | GGGCTGTATTTTTCA |
| | (PAPPA), mRNA | AAGGTGGGGAGAGTG |
| | | GAGCACACACCTTGA |
| SEQ ID | ref \| NG_029387.2\| Homo sapiens | CACATGCATGATTGT |
| NO: 3706 | V-set pre-B cell surrogate light chain 1 | AGGTGGAACCAGAAG |
| | (VPREB1), | AACCACCCAGGCAAC |
| | | TCACAGAACTGGGGA |
| SEQ ID | GI 300796044 NG_023367 Homo sapiens | GGCCCTCCATCTCCT |
| NO: 3707 | platelet derived growth factor receptor beta | ACTATGTACAATGAG |
| | (PDGFRB), RefSeqGene on chromosome 5 | AGCAAGACCATCCCC |
| | | CAGGAACTGCCTAGC |
| SEQ ID | GI 188497624 NM_012205 Homo sapiens | TCATGTTCATCGGAG |
| NO: 3708 | 3-hydroxyanthranilate 3,4-dioxygenase | GCCCCAACACCAGGA |
| | (HAAQ), mRNA | AGGACTATCACATCG |
| | | AAGAGGGTGAAGAGG |
| SEQ ID | GI 544583587 NM_004683 Homo sapiens | AGAACTGCCGGTGTG |
| NO: 3709 | regucalcin | GTGAGTCTCCAGTAT |
| | (RGN), transcript variant 1, mRNA | GGGAGGAAGTGTCCA |
| | | ACTCTCTGCTCTTTG |
| SEQ ID | GI 227496937 NG_011931 Homo sapiens | CCAGAGTTTTTGTTT |
| NO: 3710 | cholinergic receptor nicotinic alpha 4 subunit | CCTTTGAAGCAGCAA |
| | (CHRNA4), RefSeqGene on chromosome 20 | TTTCAGGAGCAGCAG |
| | | AGGCAAGGGCCAGGA |
| SEQ ID | GI 537544658 NG_033791 Homo sapiens | GTTAGGGGTTAAAAT |
| NO: 3711 | gremlin 1, DAN family BMP antagonist | CCCCCTCTTAAACGG |
| | (GREM1), RefSeqGene on chromosome 15 | TCCGAGGGCAGAGAG |
| | | GTGACCACCGATAGG |
| SEQ ID | GI 197209823 NG_008387 Homo sapiens | GTCCCCAGCCATTGC |
| NO: 3712 | interleukin 1 receptor associated kinase 1 | TTAAGAAGTGCAGGT |
| | (IRAK1), RefSeqGene on chromosome X | CACGGAGACTTTGCT |
| | | CCTCGTTTTCCAGAA |
| SEQ ID | GI 62122910 NM_001014336 Homo sapiens | AATCTCAGGGACCCC |
| NO: 3713 | interleukin 31 | TCATATGGGCTAGTC |
| | (IL31), mRNA | CCGGGAGGGCTGAGA |
| | | TGTGAATTTGTGAAT |
| SEQ ID | GI 1041818013 NM_006065 Homo sapiens | TGCTGCCACAGCCCA |
| NO: 3714 | signal regulatory protein beta 1 | ACATTGAGGAGCCCT |
| | (SIRPB1), transcript variant 1, mRNA | CGATGACTGCCCTTT |
| | | ACAACTCATATTCAG |
| SEQ ID | GI 117606537 EF064743 Homo sapiens | GATAATCAGGTCTCA |
| NO: 3715 | chemokine | CCAAACCTGGCAGCC |
| | (C-X-C motif) ligand 13 | TTCCACTGTACGTGT |
| | (B-cell chemoattractant) | GGGCAAAGACCAACA |
| | (CXCL13) gene, complete cds | |
| SEQ ID | GI 31164 X51956 Human ENO2 gene for neuron specific | GGGGATGAAGCTCGC |
| NO: 3716 | (gamma) enolase | TTTGCCGGACATAAC |
| | | TTCCGTAATCCCAGT |
| | | GTGCTGTGATTCCTC |
| SEQ ID | GI 238776812 NG_012249 Homo sapiens | AAGTTGATCAAAGAA |
| NO: 3717 | aldehyde dehydrogenase 1 family member A1 | GCTGCCGGGAAAAGC |
| | (ALDH1A1), RefSeqGene on chromosome 9 | AATCTGAAGAGGGTG |
| | | ACCCTGGAGCTTGGA |
| SEQ ID | GI 347446697 NG_030008 Homo sapiens | CTCCCAAAAACATCA |
| NO: 3718 | colony stimulating factor 1 | GGGCCCCCATCACTC |
| | (CSF1), RefSeqGene on chromosome 1 | CAGAAGAAAGCCAAG |
| | | GTTGTCTAGTCTGGG |
| SEQ ID | GI 699969198 NG_041780 Homo sapiens | GGAAAAGGCTGCAGA |
| NO: 3719 | NLR family CARD domain containing 4 | AGACACAGGTGGAAT |
| | (NLRC4), RefSeqGene on chromosome 2 | CCACATGGAAGAGGC |
| | | CCCAGAAACCTACAT |
| SEQ ID | GI 1150561157 NG_052945 Homo sapiens | GAGAGTTCTCAATGC |
| NO: 3720 | succinate-CoA ligase GDP-forming beta subunit | ATGGCAGCTGGCTTT |
| | (SUCLG2), RefSeqGene on chromosome 3 | CCCAGTGTGAGTGAC |
| | | CCAAGACCAAGGTAG |
| SEQ ID | GI 523498696 NM_002981 Homo sapiens | GCCCTACACCATTCC |
| NO: 3721 | C-C motif chemokine ligand 1 | TTCTGCCCTGCTTTT |
| | (CCL1), mRNA | GCTAGGTCACAGAGG |
| | | ATCTGCTTGGTCTTG |
| SEQ ID | GI 149192859 NM_012192 Homo sapiens | CACAATCCGTTGTTC |
| NO: 3722 | translocase of inner mitochondrial membrane 10B | TGTCGTTTAGTGCAT |
| | (TIMM10B), mRNA | ATCTGCTGGCTTCAG |
| | | CCCTGGCAGCTGAGA |
| SEQ ID | GI 219521814 NG_009215 Homo sapiens | AGGTCTAAAGTGGAG |
| NO: 3723 | bone morphogenetic protein 4 | AGCAATGCCATTTTA |
| | (BMP4), RefSeqGene on chromosome 14 | ATTCTCCCTCCCCCA |
| | | CCCCTTTTCACCCCC |
| SEQ ID | GI 334848115 NG_029039 Homo sapiens | AAGCTGTGGAAGTCA |
| NO: 3724 | major histocompatibility complex, class 1, G | GCGGTTGAACCTATT |
| | (HLA-G), RefSeqGene on chromosome 6 | TTTCCTAATGCTCCC |
| | | CTCAGCACTGCCCTC |
| SEQ ID | GI 1028224165 NG_047209 Homo sapiens | CCTTTGGCAAATGCC |
| NO: 3725 | interleukin 33 | ACCTGAGAAGCCATG |
| | (IL33), RefSeqGene on chromosome 9 | CCAGCCCTGGAAACA |
| | | CTTTGAATCAGATGA |
| SEQ ID | GI 237858699 NG_012094 Homo sapiens | CCAACAGACAACCCC |
| NO: 3726 | kallikrein 1 | AAAGTCTGGCACATC |
| | (KLK1), RefSeqGene on chromosome 19 | CTCAAAATCACCCCT |
| | | ACTCACCTCCCCAAA |
| SEQ ID | GI 346227162 NG_029971 Homo sapiens | AAACACTTTTCTAAT |
| NO: 3727 | RELA proto-oncogene, NF-kB subunit | CAGGGCCAGCCACTG |
| | (RELA), RefSeqGene on chromosome 11 | GAGAAACTGAAATGA |
| | | GTGGGCGGGGTGCTA |
| SEQ ID | GI 209447083 NG_008732 Homo sapiens | GACCTCACAGGAAGC |
| NO: 3728 | vascular endothelial growth factor A | AGTGGACTGGCCTGT |
| | (VEGFA), RefSeqGene on chromosome 6 | TTCCCCACTGTTCTT |
| | | TCTGTTTTCACACCT |
| SEQ ID | GI 164695701 AK315511 Homo sapiens | ACAAGAGCACCATCG |
| NO: 3729 | cDNA, FLI96576, Homo sapiens | GCAGGGACTTGATAG |
| | annexin A4 | ACGACCTGAAGTCAG |
| | (ANXA4), mRNA | AACTGAGTGGCAACT |
| SEQ ID | GI 747811824 NM_017594 Homo sapiens | CAACAGTAAGCCACC |
| NO: 3730 | DIRAS family GTPase 2 | CACTCTGGAGTCACA |
| | (DIRAS2), mRNA | GAATCTGCCAGGCGG |
| | | TTCAAGTGAAAACCA |
| SEQ ID | GI 58530885 NM_001570 Homo sapiens | TGCAGTTCTTGACTC |
| NO: 3731 | interleukin 1 receptor associated kinase 2 | ACCTCTCTGGGCCTC |
| | (IRAK2), mRNA | AGGTTCTACAAATGC |
| | | CAGACACCTAGCGAA |
| SEQ ID | GI 402794166 NM_002382 Homo sapiens | TAAAAAGGAAGTGGG |
| NO: 3732 | MYC associated factor X | GACAGATTCGCAGCA |
| | (MAX), transcript variant 1, mRNA | CAGAGTCGCTGGCAT |
| | | GTTTCACTCCTGCTT |
| SEQ ID | GI 1036032480 AH002739 Homo sapiens | ATGTCCCTGTTTCCT |
| NO: 3733 | cholecystokinin | TCCTCCCTGCTCCTT |
| | (CCK) gene, complete cds | GTAGCTCCTTCTGGA |
| | | CGAATGTCCATCGTT |
| SEQ ID | GI 238018114 NG_012216 Homo sapiens | AGGGAATGATCCCTT |
| NO: 3734 | B-cell CLL/lymphoma 10 | ATCTCTGGTTCCCTC |
| | (BCL10), RefSeqGene on chromosome 1 | ATCAGCCCCAGCAAT |
| | | GTCCTAGTCTTGGGG |
| SEQ ID | GI 189409117 NG_007972 Homo sapiens | AGAGCCCTAAAAAGC |
| NO: 3735 | cytochrome P450 family 2 subfamily C member 8 | TTCTCAGCAGAGCTT |
| | (CYP2C8), RefSeqGene on chromosome 10 [symbol] | AGCCTATCTGCATGG |
| | | CTGCCAAGTGTTGCA |
| SEQ ID | GI 628601932 NG_034128 Homo sapiens | CTCCCTTGGGTCGGA |
| NO: 3736 | guanylate cyclase 1 soluble subunit alpha | GTCATGAGCCTGTGA |
| | (GUCY1A3), RefSeqGene on chromosome 4 | TTGCCTACACCTGCG |
| | | TGTTATTAGGTCTCA |
| SEQ ID | GI 301336145 NG_023427 Homo sapiens | GCAGATTCCTGAGAT |
| NO: 3737 | myocyte enhancer factor 2C | TGCAGCTAAGTGGGC |
| | (MEF2C), RefSeqGene on chromosome 5 | CTGAGGTGGGGCCCA |
| | | TGAATCTGGATTTTA |
| SEQ ID | GI 385275063 NG_032144 Homo sapiens | ACCCCAAAAGGCTTT |
| NO: 3738 | transient receptor potential | CAGCCAAAACGTCTG |
| | cation channel subfamily V member 3 | AGCCTAGGTAGGTTT |
| | (TRPV3), RefSeqGene on [symbol] | ACCAAGGGAAGCCAT |
| SEQ ID | GI 95104789 NM_005860 Homo sapiens | GGCATTCTCTGCTGG |
| NO: 3739 | follistatin like 3 | TAATTCCTGAAGAGG |
| | (FSTL3), mRNA | CATGACTGCTTTTCT |
| | | CAGCCCCAAGCCTCT |
| SEQ ID | GI 436408720 NM_014226 Homo sapiens | GTCAACAACCTACGA |
| NO: 3740 | MOK protein kinase | GAGATCCAAGCACTG |
| | (MOK), transcript variant 1, mRNA | AGGCGCCTGAATCCG |
| | | CACCCAAACATTCTT |
| SEQ ID | GI 196259787 NM_001098531 Homo sapiens | AGAAGATCCTAGAGC |
| NO: 3741 | Rap guanine nucleotide exchange factor 3 | TTCTGTTGGAGGCCA |
| | (RAPGEF3), transcript variant 1, mRNA | TGGGACCAGATTCCA |
| | | GTGCTCATGACCCAA |
| SEQ ID | GI 10799005 AF261108 Homo sapiens | GGGAACTACGGCAAA |
| NO: 3742 | type XV collagen | CTGGACAGTACTGCC |
| | (COL15A1) gene, promoter | TTAGCTGAAGGCAGC |
| | sequence and exon 1, | AGCCCCTATTCAATG |
| SEQ ID | GI 21105788 AF512686 Homo sapiens | CCTCTGTATTACCGT |
| NO: 3743 | interleukin 12B | TCCTGTCCCTAACCC |
| | (natural killer cell stimulatory | AAGTCCTTCAGTTCT |
| | factor 2, cytotoxic lymphocyte | GGGTGCTCCAGCACA |
| | maturation factor | |
| SEQ ID | GI 8101110 AF268380 Homo sapiens | AGCTGGCACGGAGCG |
| NO: 3744 | melanoma-associated delayed | TTGGGCACATCTTTG |
| | early response protein | AGATGGATGATAATG |
| | (NAB2) gene, complete cds | ACAGCCAGAAGGAAG |
| SEQ ID | GI 238814315 NG_012264 Homo sapiens | GTGTAGGATCCACTC |
| NO: 3745 | glutathione peroxidase 1 | TTTCCTTTGTGGGAA |
| | (GPX1), RefSeqGene on chromosome 3 | GTCTGGCTCCTGGCC |
| | | TGCATTTCTGCCTTC |
| SEQ ID | GI 237858687 NG_012088 Homo sapiens | CGCCAAATCCTCTTT |
| NO: 3746 | interleukin 10 | GAGCTGAGATCAGAG |
| | (IL10), RefSeqGene on chromosome 1 | GCAACAGGGCTGGAC |
| | | CCAACAGAGTTGAAA |
| SEQ ID | GI 260593670 NG_013025 Homo sapiens | TCTAGTCTTGTCTGC |
| NO: 3747 | nucleotide binding oligomerization | TCATGCTGGGAGGAA |
| | domain containing 1 | GGCGCTCATAGTCAC |
| | (NOD1), RefSeqGene on chromoso [symbol] | AATGGAGCAGGGAAA |
| SEQ ID | GI 557878734 NM_001995 Homo sapiens | GCCCATGAGCTGTTC |
| NO: 3748 | acyl-CoA synthetase long | CGGTATTTTCGAATG |
| | chain family member 1 | CCAGAGCTGGTTGAC |
| | (ACSL1), transcript variant 1, mRNA | TTCCGACAGTACGTG |
| SEQ ID | GI 38505219 NM_017888 Homo sapiens | GGGGACTTTTACATC |
| NO: 3749 | acyl-CoA synthetase medium | ACAGGGGACCGAGCT |
| | chain family member 5 | CGCATGGACAAGGAT |
| | (ACSM5), transcript variant 1, mRNA | GGCTACTTTTGGTTC |
| SEQ ID | GI 520262262 NM_002872 Homo sapiens | ACCAACGCCTTTCCC |
| NO: 3750 | ras-related C3 botulinum toxin substrate 2 | GGAGAGTACATCCCC |
| | (rho family, small GTP binding protein Rac2) | ACCGTGTTTGACAAC |
| | (RA [symbol] | TATTCAGCCAATGTG |
| SEQ ID | GI 30039658 AY273801 Homo sapiens | GCCCTGTCAGAGAGG |
| NO: 3751 | breast cancer 1, early onset | GAGGACACAATATTC |
| | (BRCA1) gene, complete cds | TCTCCTGTGAGCAAG |
| | | ACTGGCACCTGTCAG |
| SEQ ID | GI 22859017 AY148100 Homo sapiens | AGCCTAGCTTAGATA |
| NO: 3752 | colony stimulating factor 3 receptor | GCTGGGAAGGCGGAA |
| | (granulocyte) | GAATGCTCTTCCTCA |
| | (CSF3R) gene, complete cds | CTGCTCCTTTCCCTG |
| SEQ ID | GI 194018739 NG_008192 Homo sapiens | TCTGCAGAGTCTAGC |
| NO: 3753 | CD36 molecule | TATCCGCCAACAGGG |
| | (CD36), RefSeqGene on chromosome 7 | GGTGCCGTCCAAATT |
| | | CATGGCAAATAAAGG |
| SEQ ID | GI 283806588 NG_016341 Homo sapiens | ATTTTGTGCCCTTAA |
| NO: 3754 | cyclin dependent kinase inhibitor 1B | AAGCCACTGGGGATG |
| | (CDKN1B), RefSeqGene on chromosome 12 | ACGGATCCAGGATTG |
| | | TGGGTGGAGGTAGTG |
| SEQ ID | GI 225007613 NG_011612 Homo sapiens | GCTTGGAAGTGCCAG |
| NO: 3755 | upstream transcription factor 1 | CTTTACTACCCATTC |
| | (USF1), RefSeqGene on chromosome 1 | CCTTTACTGCCTCCT |
| | | TCCCATGTCAGGTGG |
| SEQ ID | GI 46361513 AY594694 Homo sapiens | TTCCAGAGGCAATGC |
| NO: 3756 | interferon gamma receptor 1 | CTGAGCAGTCTTGAG |
| | (IFNGR1) gene, complete cds | GGACATACAGGAGTT |
| | | TAGGCACTAGGGGAC |
| SEQ ID | GI 8308037 AF244139 Homo sapiens | TCTGTGGGAACTGGT |
| NO: 3757 | integrin-linked kinase 1 | GACACGGGAGGTACC |
| | (ILK) gene, complete cds | CTTTGCTGACCTCTC |
| | | CAATATGGAGATTGG |
| SEQ ID | GI 189339218 NG_007955 Homo sapiens | TTGAATGGGGAAGGA |
| NO: 3758 | cytochrome P450 family 17 | GAGGTGGAGACCACG |
| | subfamily A member 1 | CCCTCCTCCCTTGTT |
| | (CYP17A1), RefSeqGene on chromosome [symbol] | TAGAATTGTCTTCGT |
| SEQ ID | GI 282394040 NG_016265 Homo sapiens | TGGATCATGGTGGAG |
| NO: 3759 | hephaestin | GTGAGGCCAGAAAGG |
| | (HEPH), RefSeqGene on chromosome X | ACAGTTGAGCACTCA |
| | | TCCCAAAGACAGAGA |
| SEQ ID | GI 342672038 NG_029624 Homo sapiens | ACTTTTCACAGTGGC |
| NO: 3760 | NFKB inhibitor beta | CAACTTGCTAGTGAT |
| | (NFKBIB), RefSeqGene on chromosome 19 | GGTGGCTTGCTTCTG |
| | | TGCAATGGTGCCATC |
| SEQ ID | GI 170932506 NG_007486 Homo sapiens | GTGAGTGAACAAGGG |
| NO: 3761 | protein kinase AMP-activated | CAAGAAGGGAGAGGT |
| | non-catalytic subunit gamma 2 | CAAAGGCTACAGGAG |
| | (PRKAG2), RefSeqGene on chr [symbol] | ACTGACAGGGAGACC |
| SEQ ID | GI 94538326 NM_002038 Homo sapiens | GTATCGCTTTTCTTG |
| NO: 3762 | interferon alpha inducible protein 6 | TGCTACCTGCTGCTC |
| | (IFI6), transcript variant 1, mRNA | TTCACTTGCAGTGGG |
| | | GTGGAGGCAGGTAAG |
| SEQ ID | GI 325588345 HQ995528 Homo sapiens | CAAGTCATGGAGCAC |
| NO: 3763 | testicular nuclear receptor 4 | ATCTGGAAGCTGCAG |
| | (NR2C2) mRNA, complete cds | GAGTTCTGTAACAGC |
| | | ATGGCGAAGCTGGAT |
| SEQ ID | GI 308199420 NG_027788 Homo sapiens | CTCGAGCCTAGCCAA |
| NO: 3764 | MX dynamin like GTPase 1 | GCTAACACATGAAAC |
| | (MX1), RefSeqGene on chromosome 21 | ACACCATCACAGCTG |
| | | GGGAAAGGATGGCTT |
| SEQ ID | GI 1114655579 NM_002969 Homo sapiens | TGAAGGGCCTCCCCG |
| NO: 3765 | mitogen-activated protein kinase 12 | AATTGGAGAAGAAGG |
| | (MAPK12), transcript variant 1, mRNA | ATTTTGCCTCTATCC |
| | | TGACCAATGCAAGCC |
| SEQ ID | GI 269914182 NM_178859 Homo sapiens | CTGAGAGCTAGTGAG |
| NO: 3766 | solute carrier family 51 beta subunit | GGTTCAGAGAAGCCC |
| | (SLC51B), mRNA | CATCCTAAGCCAGAC |
| | | ACATGATGTGGGCTC |
| SEQ ID | GI 1036032362 AH002667 Homo sapiens | GCCATGATGTGGGCA |
| NO: 3767 | cytochrome P450 | CATGAACCCCAACCT |
| | (CYP1A2) gene, complete cds | GGGTGCACAGAGCAA |
| | | GACTCTGTATCTAAA |
| SEQ ID | GI 226492652 NG_011814 Homo sapiens | TGCCCATACCCCTGG |
| NO: 3768 | C-X-C motif chemokine receptor 1 | CCAAGGAAGATCCCT |
| | (CXCR1), RefSeqGene on chromosome 2 | TTGTCCACAAGTAAA |
| | | AGGAAATGCTCCTCC |
| SEQ ID | GI 193290114 NG_008154 Homo sapiens | TGCCAATCCAGAGCT |
| NO: 3769 | insulin receptor substrate 2 | GCTGACACCTTAAAA |
| | (IRS2), RefSeqGene on chromosome 13 | ACACTGGCCTTTAAC |
| | | ACTAGGATGGCCCCA |
| SEQ ID | GI 170014708 NG_007458 Homo sapiens | CTGCCATCATGTTGA |
| NO: 3770 | notch 1 | CACCTGAAGGTTGGA |
| | (NOTCH1), RefSeqGene on chromosome 9 | AAAACCACGGCCTTT |
| | | GTTACTGCCCAAGCA |
| SEQ ID | GI 237858748 NG_012115 Homo sapiens | GAAAAACCAGTTTGG |
| NO: 3771 | phospholipid transfer protein | CTGGAGCCGAATGCA |
| | (PLTP), RefSeqGene on chromosome 20 | AAGGGCAAGAGAGGT |
| | | GAGAGGTGAGACAGA |
| SEQ ID | GI 2828023 AF036905 Homo sapiens | ACTGCCAGGCTCCTA |
| NO: 3772 | linker for activation of T cells | CGACAGCACATCCTC |
| | (LAT) mRNA, complete cds | AGATAGTTTGTATCC |
| | | AAGGGGCATCCAGTT |
| SEQ ID | GI 53759150 NM_005063 Homo sapiens | ACCTCTCGCCATGAT |
| NO: 3773 | stearoyl-CoA desaturase | CAGACATACAGCTGC |
| | (SCD), mRNA | CTACCTAATGAGGAC |
| | | TTCAAGCCCCACCAC |
| SEQ ID | GI 80861465 NM_014331 Homo sapiens | CTCATTAGCAGTTCC |
| NO: 3774 | solute carrier family 7 member 11 | GATCTTTGTTGCCCT |
| | (SLC7A11), mRNA | CTCCTGCTTTGGCTC |
| | | CATGAACGGTGGTGT |
| SEQ ID | GI 62865623 NM_198057 Homo sapiens | TCGGCAGCTGCAGGA |
| NO: 3775 | TSC22 domain family member 3 | AAAGCTGGTCTTTGA |
| | (TSC22D3), transcript variant 1, mRNA | GAACCTCAATACCGA |
| | | CAAGCTCAACAGCAT |
| SEQ ID | GI 187473261 EU595874 Homo sapiens | GGAGCCGTCCTGAAG |
| NO: 3776 | carboxylesterase 3 | ATCAAGACACCAGTG |
| | (brain) | AAATGACCAGGGCAG |
| | (CES3) gene, complete cds | AGTCCAGGAGAAAGA |
| SEQ ID | GI 237820683 NG_012068 Homo sapiens | CTCAGTCAGTGAGGA |
| NO: 3777 | forkhead box 11 | GCAAAGCCAGGACTC |
| | (FOXIl), RefSeqGene on chromosome 5 | AAGTCTGTCTTCAGA |
| | | CTCCAAGCCTACGGC |
| SEQ ID | GI 574290111 NG_033893 Homo sapiens | AAGGAGTCTTGTCGG |
| NO: 3778 | MALTI paracaspase | AATATGGCAAAAGTG |
| | (MALT1), RefSeqGene on chromosome 18 | TTATTCGGCCTGCAG |
| | | CGGAGCAGCTGTTCA |
| SEQ ID | GI 336285218 NG_029143 Homo sapiens | TTTCCTTTCACCTCT |
| NO: 3779 | mitogen-activated protein kinase kinase 5 | GACTGTGGCCTGGTA |
| | (MAP2K5), RefSeqGene on chromosome 15 | ACCCTGGGGTTGTCC |
| | | CTGCTTTTTTCTGAC |
| SEQ ID | GI 238776808 NG_012244 Homo sapiens | TGGCTTTATGATGAT |
| NO: 3780 | SMAD family member 6 | GGGGGCATGCAGGTT |
| | (SMAD6), RefSeqGene on chromosome 15 | GGGTTGGTGCTATGG |
| | | GATAGAGTTGGGAAA |
| SEQ ID | GI 42794766 NM_203351 Homo sapiens | GTCCTACCTGCACAG |
| NO: 3781 | mitogen-activated protein kinase kinase kinase 3 | CAACATGATTGTTCA |
| | (MAP3K3), transcript variant 1, mRNA | CCGGGACATTAAGGG |
| | | AGCCAACATCCTCCG |
| SEQ ID | GI 156546893 NM_001101802 Homo sapiens | AAATGGAAGCGTGGA |
| NO: 3782 | PHD finger protein 21A | TGGTATCAGGGCCCT |
| | (PHF21A), transcript variant 1, mRNA | ACCCGTGGTCTTCTC |
| | | AGATTCTGCTAGAGC |
| SEQ ID | GI 336455032 NG_029156 Homo sapiens | TTGCTCAGGCTATTC |
| NO: 3783 | betaine-homocysteine S-methyltransferase | TCTCTGGATGGAATT |
| | (BHMT), RefSeqGene on chromosome 5 | CCCTTCCCCACCTTC |
| | | TCTGCCTGGCAGTAG |
| SEQ ID | GI 1032528811 NG_050748 Homo sapiens | CCGTGCATTACTGTT |
| NO: 3784 | solute carrier family 4 member 3 | GTCTGACCAGGTATT |
| | (SLC4A3), RefSeqGene on chromosome 2 | GAGGGGCCACCCTCT |
| | | CTCTCACAGGTGTAC |
| SEQ ID | GI 156151447 NG_006672 Homo sapiens | TGTACCCCCTTAAAG |
| NO: 3785 | actin, alpha 1, skeletal muscle | CATGTCCCTGAGTTC |
| | (ACTA1), RefSeqGene on chromosome 1 | ATCACAGAAGACTGC |
| | | TCCTCCTGTGCCCTC |
| SEQ ID | GI 237820648 NG_012041 Homo sapiens | CTGGCCCGCAGATGC |
| NO: 3786 | adenosine monophosphate deaminase 3 | TCATATGTGACAATC |
| | (AMPD3), RefSeqGene on chromosome 11 | TGGAGCTCAACTGAA |
| | | GACCAAGGTTGTCGC |
| SEQ ID | GI 335334987 NG_029076 Homo sapiens | AGGACAGATGAGTTC |
| NO: 3787 | C-X-C motif chemokine receptor 3 | CAATCTATGGAGCAC |
| | (CXCR3), RefSeqGene on chromosome X | TGACCCTACCAGTGC |
| | | AGGCAGTAGGGTTGC |
| SEQ ID | GI 260166681 NM_138387 Homo sapiens | CTTCTCTCTCCCAGA |
| NO: 3788 | glucose-6-phosphatase catalytic subunit 3 | TAAGTTGGTCCTCCC |
| | (G6PC3), transcript variant 1, mRNA | TCTGCCTTTCCTCTC |
| | | AAGCCCCCAAAGAGC |
| SEQ ID | GI 210147537 NM_025106 Homo sapiens | AAGCTCAGGACGTCA |
| NO: 3789 | splA/ryanodine receptor domain | AAAACTCACCATGGA |
| | and SOCS box containing 1 | CCCCAAGGCAGAAAC |
| | (SPSB1), mRNA | CAAGAACTGTCTGCA |
| SEQ ID | GI 343098515 NG_029659 Homo sapiens | CATGTTCACCTGTGA |
| NO: 3790 | bone morphogenetic protein 1 | TCGGTCTTCCTGAGT |
| | (BMP1), RefSeqGene on chromosome 8 | ACATGCAGGCATGGA |
| | | GGCAAACATGCACCT |
| SEQ ID | GI 994323652 NG_032159 Homo sapiens | TCAGATTTCCTCGTT |
| NO: 3791 | CASP2 and RIPK1 domain containing | GCCCCGTCTCATACT |
| | adaptor with death domain | TCCTGTTCCTCTTCT |
| | (CRADD), RefSeqGene on c [symbol] | GGGCTCTGCCAGAAC |
| SEQ ID | GI 341865607 NG_029555 Homo sapiens | CAGCGTCAGTTTCTC |
| NO: 3792 | ETS proto-oncogene 1, transcription factor | AAAAGCAGGACTCAT |
| | (ETS1), RefSeqGene on chromosome 11 | GGTTTCCCCATGCGT |
| | | AAGGTCAGCCCTTCA |
| SEQ ID | GI 342307072 NG_029607 Homo sapiens | CAGAGAATCCCTAAG |
| NO: 3793 | heat shock transcription factor 2 | TTCAGGCAGTGATGG |
| | (HSF2), RefSeqGene on chromosome 6 | CAGCAGCCCTCTCAT |
| | | GTCTAGTGCTGTCCA |
| SEQ ID | GI 306922359 NG_027701 Homo sapiens | ATGAAGTTGATGGGA |
| NO: 3794 | retinoic acid receptor alpha | GGATTACGGTGGTAA |
| | (RARA), RefSeqGene on chromosome 17 | CAGATACTGTGCAGG |
| | | TGCCCAGAGCGAGCT |
| SEQ ID | GI 405778361 NG_032938 Homo sapiens | AGACCTCCATTCCTA |
| NO: 3795 | semaphorin 3B | CTCAAGGGAGGGTAC |
| | (SEMA3B), RefSeqGene on chromosome 3 | ACATCTAGAGGACAG |
| | | AGGACAGGGCACAGC |
| SEQ ID | GI 584458494 NG_033933 Homo sapiens | GCGCTCAGCAAAGTC |
| NO: 3796 | toll like receptor 9 | TTGAGTGGCAGCAGC |
| | (TLR9), RefSeqGene on chromosome 3 | TATGGGGACTGTTCA |
| | | TTCCAAAACGGTAGA |
| SEQ ID | GI 544583587 NM_004683 Homo sapiens | GCCTGTCCTACTCCG |
| NO: 3797 | regucalcin | TGGATGCCTTTGACT |
| | (RGN), transcript variant 1, mRNA | ATGACCTGCAGACAG |
| | | GACAGATCTCCAACC |
| SEQ ID | GI 38202233 NM_052864 Homo sapiens | GTCAAGGCTCCAGGC |
| NO: 3798 | TRAP interacting protein with | AGGTTGTTACTGGTG |
| | forkhead associated domain | TTTGCAGCCTGTCAG |
| | (TIFA), mRNA | TACTTGCAGTACTGG |
| SEQ ID | GI 83026500 DQ309025 Homo sapiens | TCTCAGGCATCTACA |
| NO: 3799 | transforming growth factor | AAGGCTCTCTCTTCC |
| | beta 1 induced transcript 1 | TCCGTCAGAGTCTGG |
| | (TGFBIIl) gene, complete cds | ACTGGGTTCACTGGT |
| SEQ ID | GI 53759124 NM_001295 Homo sapiens | TGGGAGGAAAGTGTC |
| NO: 3800 | C-C motif chemokine receptor 1 | TACTAGGAGGGTGGG |
| | (CCR1), mRNA | GTGAGATTCTGTGTT |
| | | GATGTAGGAGGCCGA |
| SEQ ID | GI 208879434 NM_004895 Homo sapiens | AGCTGCTGCCTCACA |
| NO: 3801 | NLRfamily pyrin domain containing 3 | TCAGCATGTTGTCAG |
| | (NLRP3), transcript variant 1, mRNA | GATCTTGCATCAGTA |
| | | TTGAGCACCAGCCAT |
| SEQ ID | GI 1025608662 NG_047031 Homo sapiens | CTGCTATGACTGTAG |
| NO: 3802 | ATP citrate lyase | CAGGGCTTCCGAAAT |
| | (ACLY), RefSeqGene on chromosome 17 | ACCTGCCTCTCTTGG |
| | | GTGGATCACTGTGGT |
| SEQ ID | GI 259013220 NG_012921 Homo sapiens | AATGGCTGCCTGGGT |
| NO: 3803 | bisphosphoglycerate mutase | TTGGAAGGACAAGTT |
| | (BPGM), RefSeqGene on chromosome 7 | GGAACTGGAGATACC |
| | | CCTGTGAAGTGAAGC |
| SEQ ID | GI 209447084 NG_008733 Homo sapiens | AGCCACTACAGTCAA |
| NO: 3804 | copper chaperone for superoxide dismutase | ATTTCTGTTTGTGGC |
| | (CCS), RefSeqGene on chromosome 11 | CTCCCCCAACCCCTC |
| | | ACTATGGCTGATTGG |
| SEQ ID | GI 254039688 NG_012344 Homo sapiens | GGCTCTGGAGTTAGA |
| NO: 3805 | NFKB inhibitor like 1 | TTGCCTGGATTCAAA |
| | (NFKBIL1), RefSeqGene on chromosome 6 | CCCCAGCTCCAAATC |
| | | CCAGCTCCACACTTC |
| SEQ ID | GI 237858601 NG_012145 Homo sapiens | TATCTGCAGAGGATG |
| NO: 3806 | TNF receptor superfamily member 10b | GCAAGTCTTTGCTCC |
| | (TNFRSF10B), RefSeqGene on chromosome 8 | AAAACCTAAGGGCCT |
| | | GTGCTGTGGTTGACC |
| SEQ ID | GI 145312264 NM_033266 Homo sapiens | TTCTTCTTGGAGCTA |
| NO: 3807 | endoplasmic reticulum | TTGAGCCTGAGCCGA |
| | to nucleus signaling 2 | GAGAAACTTTGGGAC |
| | (ERN2), transcript variant 1, mRNA | TCCGAGCTGCATCCA |
| SEQ ID | GI 61743979 NM_017564 Homo sapiens | CACACTGACAGGAGT |
| NO: 3808 | stabilin 2 | TCTCATTCCTCCCTC |
| | (STAB2), mRNA | CATTGTCCCGATTCT |
| | | GCCCCATCGATGTGA |
| SEQ ID | GI 345110544 NG_029851 Homo sapiens | GATGGGATGGATGTC |
| NO: 3809 | phospholipase D1 | TTGATCCCATGGTGA |
| | (PLD1), RefSeqGene on chromosome 3 | GGGGATCTAGGCAGC |
| | | ACACTACAGCATCCA |
| SEQ ID | GI 308080224 NG_027757 Homo sapiens | GAAGGTGAGGACTTG |
| NO: 3810 | transforming growth | GCCCTTTTGTGGGAC |
| | factor beta receptor 3 | TGAATTGACACTAGT |
| | (TGFBR3), RefSeqGene on chromosome 1 | GTGGCTGGAGTGTGC |
| SEQ ID | GI 197304706 NM_032350 Homo sapiens | CCCCAGAGGAGCAGA |
| NO: 3811 | chromosome 7 open reading frame 50 | GGGTCCTGGAAAGGA |
| | (C7orf50), transcript variant 1, mRNA | AGCTGAAAAAGGAAC |
| | | GGAAGAAAGAGGAGA |
| SEQ ID | GI 666637974 NM_005922 Homo sapiens | CAGCTTCGACATCCA |
| NO: 3812 | mitogen-activated protein | GTCGCGGGACTGCAT |
| | kinase kinase kinase 4 | ATCCAAGAAGCTTGA |
| | (MAP3K4), transcript variant 1, mRNA | GAGGCTCGAATCTGA |
| SEQ ID | GI 1019399995 LT222231 Homo sapiens | TGGCCCCCTGATCTG |
| NO: 3813 | IncRNAfor PRTN3 gene | TGATGGCATCATCCA |
| | | AGGAATAGACTCCTT |
| | | CGTGATCTGGGGATG |
| SEQ ID | GI 196259787 NM_001098531 Homo sapiens | CCAGATGTCTTCCAG |
| NO: 3814 | Rap guanine nucleotide exchange factor 3 | ATTCTGTGCCTCTGG |
| | (RAPGEF3), transcript variant 1, mRNA | CTTTGTTGTCCAGCC |
| | | AGGCCTGCAGTTTAT |
| SEQ ID | GI 126116577 NM_176823 Homo sapiens | GCTCTAAGTGGTCCA |
| NO: 3815 | S100 calcium binding protein A7A | GGAGTCCAAGTAAGC |
| | (S100A7A), mRNA | AGTCTGGCTGGAATA |
| | | AGGCAGCATCACGGA |
| SEQ ID | GI 54781360 AY789109 Homo sapiens | TATCTGGTGATGCAG |
| NO: 3816 | interleukin 3 receptor, alpha | AGACTCTTTCCCCGC |
| | (low affinity) | ATCCCTCACATGAAA |
| | (IL3RA) gene, complete cds | GACCCCATCGGTGAC |
| SEQ ID | GI 284055256 NG_016403 Homo sapiens | GATTGTCAGGGTGGC |
| NO: 3817 | dystrophin related protein 2 | CAAGGCCAAGTATTC |
| | (DRP2), RefSeqGene on chromosome X | CTGTTCATGCCCTTG |
| | | CCACATCTCTGGTAT |
| SEQ ID | GI 393533306 NG_032816 Homo sapiens | TTTGCTGGGTTGGAG |
| NO: 3818 | 5-hydroxytryptamine receptor 1A | GCGGAGTTTATTTGT |
| | (HTRIA), RefSeqGene on chromosome 5 | TACAACCTTGGTCTG |
| | | ACCGGCAGGGATCTG |
| SEQ ID | GI 193211428 NG_008124 Homo sapiens | GAACCTCACAGAGAC |
| NO: 3819 | msh homeobox 2 | CCAGGTCAAAATCTG |
| | (MSX2), RefSeqGene on chromosome 5 | GTTCCAGAACCGAAG |
| | | GGCCAAGGCGAAAAG |
| SEQ ID | GI 22538799 NM_005408 Homo sapiens | GAAGATCTCCTTGCA |
| NO: 3820 | C-C motif chemokine ligand 13 | GAGGCTGAAGAGCTA |
| | (CCL13), mRNA | TGTGATCACCACCAG |
| | | CAGGTGTCCCCAGAA |
| SEQ ID | GI 332078493 NM_002559 Homo sapiens | ACCAAGTCGGTGGTT |
| NO: 3821 | purinergic receptor P2X 3 | GTGAAGAGCTGGACC |
| | (P2RX3), mRNA | ATCGGGATCATCAAC |
| | | CGAGTAGTTCAGCTT |
| SEQ ID | GI 332800978 NM_001206950 Homo sapiens | TACGCCTTCCCCAAG |
| NO: 3822 | solute carrier family 16 member 3 | GCCGTCAGTGTCTTC |
| | (SLC16A3), transcript variant 1, mRNA | TTCAAGGAGCTCATA |
| | | CAGGAGTTTGGGATC |
| SEQ ID | GI 211904170 NG_008937 Homo sapiens | GAGGCCTGCCTCAAA |
| NO: 3823 | CD44 molecule | TATCACTTCCCCTGT |
| | (Indian blood group) | GAAGACTGCCTGGTG |
| | (CD44), RefSeqGene on chromosome 11 | TTCTCCAGGAGAGAG |
| SEQ ID | GI 563317866 NG_033843 Homo sapiens | GCCTGGTGAAGATGT |
| NO: 3824 | DnaJ heat shock protein family | GAAGACCGATACCAG |
| | (Hsp40) member C6 | ACACCAAAGGGCTGC |
| | (DNAJC6), RefSeqGene on chromosome [symbol] | ATTGCCTTCTTCCAA |
| SEQ ID | GI 297747369 NG_021416 Homo sapiens | CAGAGGGTCAAGTCT |
| NO: 3825 | growth hormone releasing hormone receptor | GCTTCCTTGCCTACA |
| | (GHRHR), RefSeqGene on chromosome 7 | TAGCCATGAACTTCC |
| | | CAGATCTAGGCGCCA |
| SEQ ID | GI 257153317 NG_012804 Homo sapiens | AACATCCATGTGACG |
| NO: 3826 | mannosidase beta | GGAAGCCATGGGACA |
| | (MANBA), RefSeqGene on chromosome 4 | GGGTGTAGAACAAGG |
| | | AGAAGCCTGAGAGAG |
| SEQ ID | GI 212549770 NG_008989 Homo sapiens | TGGATGGGAGTGTTG |
| NO: 3827 | matrix metallopeptidase 2 | GGATTTTGAGCTAGG |
| | (MMP2), RefSeqGene on chromosome 16 | GAACTTGGTTTTGGT |
| | | GACCCAGGGCATGAG |
| SEQ ID | GI 340805876 NG_029513 Homo sapiens | GCAGTTTTGGGACTG |
| NO: 3828 | ribosomal protein 56 kinase B1 | GGCAGCTATGGAGGT |
| | (RPS6KB1), RefSeqGene on chromosome 17 | CACATCATTCCTTTG |
| | | CCCTTAGGCTTGAGT |
| SEQ ID | GI 574957130 NM_001815 Homo sapiens | CATTTACTGCCGGAT |
| NO: 3829 | carcinoembryonic antigen | GGACCACAAAGCAGA |
| | related cell adhesion molecule 3 | AGTGGCTTCTTAGCT |
| | (CEACAM3), transcript variant 1 [symbol] | TCCTCCAGGAGCTGC |
| SEQ ID | GI 197313788 NM_001423 Homo sapiens | ATGGAACGCAGTATC |
| NO: 3830 | epithelial membrane protein 1 | ACCACGGCTATTCCT |
| | (EMP1), mRNA | ACATCCTGGGCTGGA |
| | | TCTGCTTCTGCTTCA |
| SEQ ID | GI 188497624 NM_012205 Homo sapiens | GCAGACTCAGTGCAG |
| NO: 3831 | 3-hydroxyanthranilate 3,4-dioxygenase | CACTTCCACACCAAG |
| | (HAAQ), mRNA | AAGGCCCTCAATAAA |
| | | GGCTTCCTGAGGAAC |
| SEQ ID | GI 23510408 NM_003057 Homo sapiens | TGCTGACTCCTGGAA |
| NO: 3832 | solute carrier family 22 member 1 | GCTGGACCTCTTTCA |
| | (SLC22A1), transcript variant 1, mRNA | GTCCTGTTTGAATGC |
| | | GGGCTTCTTGTTTGG |
| SEQ ID | GI 8101110 AF268380 Homo sapiens | TCTTACCCATCTTTT |
| NO: 3833 | melanoma-associated | CAGGCTTCACCCTGA |
| | delayed early response protein | AGAACTGGGAGGCCC |
| | (NAB2) gene, complete cds | TCCACTGAAGAAGCT |
| SEQ ID | GI 82583657 DQ297142 Homo sapiens | CTCAGAGAGGCTGTC |
| NO: 3834 | peroxiredoxin 1 | AGCTGGGAAAAGTTA |
| | (PRDX1) gene, complete cds | TTCTCCTGGGCCCTT |
| | | GAGGCCAAGGTAGAT |
| SEQ ID | GI 299782537 NG_023178 Homo sapiens | GGGGATTGGGGGGTT |
| NO: 3835 | CD14 molecule | GGATAGTGCAGAGTA |
| | (CD14), RefSeqGene on chromosome 5 | TGGTACTGGCCTAAG |
| | | GCACTGAGGATCATC |
| SEQ ID | GI 1145140176 NG_008729 Homo sapiens | TCCGGGGAGAGGCAC |
| NO: 3836 | superoxide dismutase 2 | ATGAGGTGAAATGTT |
| | (SOD2), RefSeqGene on chromosome 6 | TTCCCCCTCCCTTCC |
| | | TTGTTGTCTCTTACT |
| SEQ ID | GI 38505219 NM_017888 Homo sapiens | CTTCGACCCTAAAGA |
| NO: 3837 | acyl-CoA synthetase medium | TGTCAAAGGTGTGCA |
| | chain family member 5 | GCTTCCAAACGGCAT |
| | (ACSM5), transcript variant 1, mRNA | CCCCAGGATCACTGG |
| SEQ ID | GI 50659075 NM_001638 Homo sapiens | GTCTCACTTCTCAAT |
| NO: 3838 | apolipoprotein F | GACTGGACTGTGTGG |
| | (APOF), mRNA | GTACTCTGCTCCAGA |
| | | CATGCGTGGCCTCAG |
| SEQ ID | GI 164695818 AK315628 Homo sapiens | GGCTCACCTTTGCCG |
| NO: 3839 | cDNA, FU96713, Homo sapiens | ACTGCGGGTTCGAAA |
| | ankyrin repeat and SOCS box-containing 2 | GGCCATTGGGAAATA |
| | (ASB2), mRNA | CCGTATAAAACTCCT |
| SEQ ID | GI 2660728 AFO31523 Homo sapiens | GAGGATGAGTGACGA |
| NO: 3840 | bcl-xL/bcl-2 associated death promoter | GTTTGTGGACTCCTT |
| | (BAD) mRNA, complete cds | TAAGAAGGGACTTCC |
| | | TCGCCCGAAGAGCGC |
| SEQ ID | GI 167555126 NM_005582 Homo sapiens | TTCCTGGAACTGGGC |
| NO: 3841 | CD180 molecule | AGGGACTCACTGTGC |
| | (CD180), mRNA | TTTTCTGAGCTTCTT |
| | | ACTCCTGTGAGTCCC |
| SEQ ID | GI 49574531 NM_019884 Homo sapiens | ACTACCGGGCCCCAG |
| NO: 3842 | glycogen synthase kinase 3 alpha | AGCTCATCTTTGGAG |
| | (GSK3A), mRNA | CCACTGATTACACCT |
| | | CATCCATCGATGTTT |
| SEQ ID | GI 151101190 NM_006435 Homo sapiens | AACCCGATCACCGCT |
| NO: 3843 | interferon induced transmembrane protein 2 | GGTCACCATGAACCA |
| | (IFITM2), mRNA | CATTGTGCAAACCTT |
| | | CTCTCCTGTCAACAG |
| SEQ ID | GI 392050748 NM_001267774 Homo sapiens | CTGAACAAGCTGAGG |
| NO: 3844 | intraflagellar transport 20 | GTGTTGGACCCAGAG |
| | (IFT20), transcript variant 1, mRNA | GTTACCCAGCAGACC |
| | | ATAGAGCTGAAGGAA |
| SEQ ID | GI 169790829 NM_001754 Homo sapiens | TTCACCAGGCTGAGA |
| NO: 3845 | runt related transcription factor 1 | CCCTACCCTATGGGG |
| | (RUNX1), transcript variant 1, mRNA | TATATGGGCTTTTAC |
| | | CTGTGCACCAGTGTG |
| SEQ ID | GI 117606575 EF064762 Homo sapiens | TGGGAAATGGCTATC |
| NO: 3846 | chemokine | CTAGATGAGGAAGGA |
| | (C-C motif) ligand 27 | GTCTGTGGCAACAGG |
| | (CCL27) gene, complete cds | CCGAGAATTAGGGCC |
| SEQ ID | GI 94717615 DQ523828 Homo sapiens | GCCATGCAAGGAGGT |
| NO: 3847 | transglutaminase 2 | GTGTGCACGACATAC |
| | (C polypeptide, protein-glutamine- | TAGGCCAAGGGAACT |
| | gamma-glutamyltransferase) | ATGCAAGTGAAAGCT |
| | (TGM2) ge [symbol] | |
| SEQ ID | GI 18073363 AJ277883 Homo sapiens | ACGAGAGCGGGGCCA |
| NO: 3848 | mRNA for sialidase | GGACCTCCTATGATG |
| | (NEU4 gene) | AGATTTCCTTTTGTA |
| | | CATTCTCCCTGCGTG |
| SEQ ID | GI 423091980 JX512648 Homo sapiens | AAGAGTCCACCAGGC |
| NO: 3849 | SH2 domain containing 1A | CATGGACTCAGTGGC |
| | (SH2D1A) mRNA, complete cds | TGTGTATCATGGCAA |
| | | AATCAGCAGGGAAAC |
| SEQ ID | GI 117606567 EF064758 Homo sapiens | AGACAGGGATGATCT |
| NO: 3850 | chemokine | GACTCAAGGCCACTG |
| | (C-C motif) receptor 7 | ACCAGGCTCCCTTTT |
| | (CCR7) gene, complete cds | GCCACATACAAGAGT |
| SEQ ID | GI 966209932 NG_033919 Homo sapiens | GACATCTCCAGGTGG |
| NO: 3851 | dedicator of cytokinesis 1 | GCCACACTTTAGCAT |
| | (DOCK1), RefSeqGene on chromosome 10 | TTCAGTAGGTGCCAT |
| | | GTGTTGCAGTGCTCA |
| SEQ ID | GI 225637493 NG_011688 Homo sapiens | GGCCCTCAGACCAGT |
| NO: 3852 | growth hormone receptor | AGCCAGTATGAAACT |
| | (GHR), RefSeqGene on chromosome 5 | GAAGCCTCTTGACCA |
| | | CAGCCTCCATGAGAG |
| SEQ ID | GI 341865570 NG_029565 Homo sapiens | TGGGCAGTCAGGAAT |
| NO: 3853 | interleukin 24 | CCTGGGAGCTAGGCT |
| | (IL24), RefSeqGene on chromosome 1 | GTGTCTGCCACTGAT |
| | | TGACTATGTGAATCG |
| SEQ ID | GI 345525590 NG_029936 Homo sapiens | GCCTGATAGAAAGAA |
| NO: 3854 | mitogen-activated protein kinase 3 | GGCATGCGCACCTGG |
| | (MAPK3), RefSeqGene on chromosome 16 | GCTCAAACTCCAGCT |
| | | GTGCCACTTAGTAAG |
| SEQ ID | GI 254911084 NG_012516 Homo sapiens | CCAGTGTAGGGGAGG |
| NO: 3855 | moesin | GATGGTGAATGCCTA |
| | (MSN), RefSeqGene on chromosome X | CCTTCTAAATGGCAC |
| | | TCACTGTGAGGTGCT |
| SEQ ID | GI 259013237 NG_012931 Homo sapiens | AATGCTTCCTGCCTA |
| NO: 3856 | ubiquitin C-terminal hydrolase Li | TACTGTGATCCTCAC |
| | (UCHL1), RefSeqGene on chromosome 4 | AAAAGGGCTCTGTCC |
| | | TGTCTGGTGCCAGGA |
| SEQ ID | GI 116006947 NM_001285 Homo sapiens | GCTGTCTCTCAGCGC |
| NO: 3857 | chloride channel accessory 1 | TCCATCCAGCTTGAG |
| | (CLCA1), mRNA | AGTAAGGGATTAACC |
| | | CTCCAGAACAGCCAG |
| SEQ ID | GI 390979633 NM_004135 Homo sapiens | CAGAGCTCATGCTGC |
| NO: 3858 | isocitrate dehydrogenase 3 | ATGTCAAGTCCGTCT |
| | (NAD(+)) gamma | TCAGGCACGCATGTG |
| | (IDH3G), transcript variant 1, mRNA | TACCAGTGGACTTTG |
| SEQ ID | GI 37577125 NM_194315 Homo sapiens | TCGCATTAAGAAAGA |
| NO: 3859 | ubiquitin conjugating enzyme E2 J2 | CCCGGTGCCTTACAT |
| | (UBE2J2), transcript variant 1, mRNA | CTGTGCCGAGCCCCT |
| | | CCCTTCGAATATTCT |
| SEQ ID | GI 48255965 NM_006759 Homo sapiens | GTGGTGAAACTCAAT |
| NO: 3860 | UDP-glucose pyrophosphorylase 2 | GGTGGTTTGGGAACC |
| | (UGP2), transcript variant 1, mRNA | AGCATGGGCTGCAAA |
| | | GGCCCTAAAAGTCTG |
| SEQ ID | GI 1049010878 AH003215 Homo sapiens | AGACCTGACTCTCTT |
| NO: 3861 | CD8 antigen | AAGGAGGCTGAGAGC |
| | (CD8A) gene, complete cds, | CCAAACTGCTGTCCC |
| | alternatively spliced | AAACATGCACTTCCT |
| SEQ ID | GI 328751683 NG_028290 Homo sapiens | GAGAGATTGCTCTGG |
| NO: 3862 | acyl-CoA synthetase long | CACTGGCAGATCAAG |
| | chain family member 6 | CAGATGGAGTGGAGA |
| | (ACSL6), RefSeqGene on chromosome 5 | CAGAGTGTGTGCAGA |
| SEQ ID | GI 1025608652 NG_047034 Homo sapiens | GGATTGCGAGCTGGC |
| NO: 3863 | autocrine motility factor receptor | TGCCGAGTTTTTCGG |
| | (AMFR), RefSeqGene on chromosome 16 | TTGAAGTGATGCACA |
| | | CCACCAACATTCTTG |
| SEQ ID | GI 359465544 NG_031854 Homo sapiens | AGATTCTGCCTCAGA |
| NO: 3864 | glutamate ionotropic | AAGGACGTGGGTTGG |
| | receptor NMDAtype subunit 2B | TGACAATGGCATGGG |
| | (GRIN2B), RefSeqGene on chromoso [symbol] | TTAAAAAGCGGGTCA |
| SEQ ID | GI 307133762 NG_027719 Homo sapiens | GAATCACCTGAGGGT |
| NO: 3865 | glutathione-disulfide reductase | GCCTGGATTCCCGAC |
| | (GSR), RefSeqGene on chromosome 8 | TTAATTGGTCTGAGG |
| | | TGCACTTGGGCTTTG |
| SEQ ID | GI 325910867 NM_004874 Homo sapiens | CTTTTGGAACTGGAT |
| NO: 3866 | BCL2 associated athanogene 4 | TCAGTTGAAACTGGG |
| | (BAG4), transcript variant 1, mRNA | GGCCAGGACTCTGTA |
| | | CGGCAGGCCAGAAAA |
| SEQ ID | GI 332000013 NM_002733 Homo sapiens | CTGTGTTTGTGCATA |
| NO: 3867 | protein kinase AMP-activated | TGAAGGGAGATGGGG |
| | non-catalytic subunit gamma 1 | GTTAGGTGGAGGAGG |
| | (PRKAG1), transcript variant 1 [symbol] | AGAGCAGCGTGGTTA |
| SEQ ID | GI 117606535 EF064742 Homo sapiens | GACACCCACTTGTCT |
| NO: 3868 | chemokine | CGTGGTTCGCTCACT |
| | (C-X-C motif) ligand 16 | TCCTTGTCCCCTACC |
| | (CXCL16) gene, complete cds | TTTTCCGTCTGTCAT |
| SEQ ID | GI 20530138 AF261937 Homo sapiens | GGAGAGGGGACTTTT |
| NO: 3869 | NFKBIE protein | GACATTGCCGTCTTT |
| | (NFKBIE) gene, complete cds | TGTTAGCCTCTCAGC |
| | | CCCAGCCTTTTCCAT |
| SEQ ID | GI 1032528813 NG_050736 Homo sapiens | GTACCTCTTTCTGTG |
| NO: 3870 | complement C3a receptor 1 | GCGGCGAGTCCTCTG |
| | (C3AR1), RefSeqGene on chromosome 12 | AAAATCTGGTGGGGA |
| | | GAGTGGATGAAGCTT |
| SEQ ID | GI 806904734 NG_008957 Homo sapiens | TTCCAGCAATGACCC |
| NO: 3871 | monoamine oxidase A | AGAGGGATGGTGGGG |
| | (MAOA), RefSeqGene on chromosome X | AGACAGGAAACATAG |
| | | ATGGTGAGGCAAGAT |
| SEQ ID | GI 338827642 NG_029226 Homo sapiens | GGCCAAGCCCAGGTG |
| NO: 3872 | nuclear factor of activated T-cells 1 | CAGTATGTTCGTGTG |
| | (NFATC1), RefSeqGene on chromosome 18 | TTTAAGCTTCTTAGA |
| | | TGCTGGGGTGTGGAA |
| SEQ ID | GI 212549746 NG_008982 Homo sapiens | AGGGCATGTTGCATA |
| NO: 3873 | solute carrier family 22 member 5 | ACAAAACAAGCAGCA |
| | (SLC22A5), RefSeqGene on chromosome 5 | TCAAACTTGGGGGAG |
| | | AAGGAAGGCAGCACG |
| SEQ ID | GI 1015539575 AH009651 Homo sapiens | TCTGGCTTTGCTGCT |
| NO: 3874 | xeroderma pigmentosum group C protein | CTGTGGCCATGGCAA |
| | (XPC) gene, complete cds | GTTTCTTACTCTCTC |
| | | TGAGCCTTTGCATCC |
| SEQ ID | GI 88698227 DQ400449 Homo sapiens | GCAGCAGTGGACAGT |
| NO: 3875 | complement component 5 | GCTGTGTATGGAGTC |
| | (C5) gene, complete cds | CAAAGAGGAGCCAAA |
| | | AAGCCCTTGGAAAGA |
| SEQ ID | GI 117606491 EF064720 Homo sapiens | TTGGAAAAACCTGGA |
| NO: 3876 | interleukin 27 | ATCCAGTCCTGGCTT |
| | (IL27) gene, complete cds | CCCACCTTCTGGCTA |
| | | TTTGACCTTGGGCAA |
| SEQ ID | GI 225543278 NG_011648 Homo sapiens | AAAAGATTCCCAAGC |
| NO: 3877 | angiotensin 1 converting enzyme | CAACTGAGTTACCTC |
| | (ACE), RefSeqGene on chromosome 17 | ACGTGGGTCAGGTCT |
| | | CTGTGGCTCTCTGCA |
| SEQ ID | GI 212549770 NG_008989 Homo sapiens | CAGAGGTGTTAGGTG |
| NO: 3878 | matrix metallopeptidase 2 | CTTTTGTGAGCACGT |
| | (MMP2), RefSeqGene on chromosome 16 | CCCAGTCAAGGAAGG |
| | | CTTCACAGATGCAGC |
| SEQ ID | GI 393537030 NM_000151 Homo sapiens | CGCAGACCTCAGGAA |
| NO: 3879 | glucose-6-phosphatase catalytic subunit | TGCCTTCTACGTCCT |
| | (G6PC), transcript variant 1, mRNA | CTTCCCCATCTGGTT |
| | | CCATCTTCAGGAAGC |
| SEQ ID | GI 117606569 EF064759 Homo sapiens | TACAAGCGGCTCAGG |
| NO: 3880 | chemokine | TCCATGACTGATGTG |
| | (C-C motif) receptor 4 | TACCTGCTCAACCTT |
| | (CCR4) gene, complete cds | GCCATCTCGGATCTG |
| SEQ ID | GI 22001412 AF531102 Homo sapiens | GCCCTGCAAACTTTT |
| NO: 3881 | interleukin 1 receptor, type 1 | CTACCCAGGTTCTCA |
| | (IL1R1) gene, complete cds | GCTCCATCTCCTCAA |
| | | CTCACTGGGCTTTGC |
| SEQ ID | GI 306482672 NG_027674 Homo sapiens | AACCGGCCTTGTGAC |
| NO: 3882 | DNA damage inducible transcript 3 | AGTTTCTACTTAAAC |
| | (DDIT3), RefSeqGene on chromosome 12 | TCTGGCTGCCCCCAT |
| | | GAGGGTATGACAGGA |
| SEQ ID | GI 340805836 NG_029500 Homo sapiens | AGCCACTGCCCTAGA |
| NO: 3883 | fatty acid binding protein 6 | TCAAACCTGTAGATG |
| | (FABP6), RefSeqGene on chromosome 5 | TTGGAGTCGGACCCT |
| | | TTTTCTGTCCTGGGG |
| SEQ ID | GI 340138904 NG_029442 Homo sapiens | AAAAGCGTGGCAGCT |
| NO: 3884 | MLX, MAX dimerization protein | GCTTTTAGATCTTAA |
| | (MLX), RefSeqGene on chromosome 17 | GGGCGTTTCTACAGC |
| | | TTTGAGGGCCCTGGT |
| SEQ ID | GI 217035112 NM_152672 Homo sapiens | TTCCCTGGTGCTGGT |
| NO: 3885 | solute carrier family 51 alpha subunit | GGAAATGACCATCAC |
| | (SLC51A), mRNA | CTCGTTTTATGCCGT |
| | | GTGCTTTTACCTGCT |
| SEQ ID | GI 2246432 U45984 Homo sapiens | GGTACATCGCCATTG |
| NO: 3886 | CCR6 chemokine receptor | TACAGGCGACTAAGT |
| | (CMKBR6) gene, complete cds | CATTCCGGCTCCGAT |
| | | CCAGAACACTACCGC |
| SEQ ID | GI 1049010853 AH002961 Homo sapiens | TGAAATGACTGAGTA |
| NO: 3887 | N-ras oncogene | CAAACTGGTGGTGGT |
| | (NRAS) gene, complete cds | TGGAGCAGGTGGTGT |
| | | TGGGAAAAGCGCACT |
| SEQ ID | GI 190194387 NG_008002 Homo sapiens | ACCCTGTTTTTCTGC |
| NO: 3888 | acyl-CoA dehydrogenase, long chain | CCAAATGTTGCCTTT |
| | (ACADL), RefSeqGene on chromosome 2 | TTGGCTTGCCCCTGC |
| | | CTCTATCTTATGCCC |
| SEQ ID | GI 635172888 NG_007045 Homo sapiens | TGGGCAACTCCTTGG |
| NO: 3889 | acyl-CoA dehydrogenase, | GACATGTGGTGCACA |
| | C-4 to C-12 straight chain | TACCAGCAAATGATC |
| | (ACADM), RefSeqGene on chromosome | TGCCCTTTAAGGGAA |
| SEQ ID | GI 299116025 NG_023038 Homo sapiens | AGTTGGACTGACTCC |
| NO: 3890 | autophagy related 16 like 1 | AGTACTGGCCTAATA |
| | (ATG16L1), RefSeqGene on chromosome 2 | ACTGGCAGCGGGTAA |
| | | GTGAAAGGAGCCAGC |
| SEQ ID | GI 359465544 NG_031854 Homo sapiens | GCATTGTGACCCTGG |
| NO: 3891 | glutamate ionotropic receptor | AGGAGGCACCATTTG |
| | NMDA type subunit 2B | TCATTGTGGAAAGTG |
| | (GRIN2B), RefSeqGene on chromoson [symbol] | TGGACCCTCTGAGTG |
| SEQ ID | GI 257153311 NG_012800 Homo sapiens | GCGGGTTGGCTTTTT |
| NO: 3892 | regulator of G protein signaling 2 | GTCCTCCTGCATTCA |
| | (RGS2), RefSeqGene on chromosome 1 | AAATGACATCAGAAT |
| | | CCTGCGGCTGAAGCG |
| SEQ ID | GI 315365062 HQ384162 Homo sapiens | CCCTGTGTAGACCCG |
| NO: 3893 | cell death-inducing DFFA-like effector a | AATCCTGAGTGCTGC |
| | (CIDEA) gene, complete cds | AAGATCTGTATGTGG |
| | | CACAAGCCACTCAGA |
| SEQ ID | GI 336285267 NG_029154 Homo sapiens | AAAGGGAGGGGAGAG |
| NO: 3894 | interferon alpha 2 | AAAAGCCGCAAAGGA |
| | (IFNA2), RefSeqGene on chromosome 9 | AACATCCACAGCCAC |
| | | AAAGGAATGGCTGTA |
| SEQ ID | GI 344925862 NG_029825 Homo sapiens | AAATCAAGGAGCACC |
| NO: 3895 | ubiquitin specific peptidase 2 | CTATAGAAACTGCCC |
| | (USP2), RefSeqGene on chromosome 11 | TGGGCCCATCCACTT |
| | | CTTCTCCACCAGTCA |
| SEQ ID | GI 210032511 NG_008871 Homo sapiens | TCCATGGACATCATG |
| NO: 3896 | xanthine dehydrogenase | GTGTGGGTGGCCTCT |
| | (XDH), RefSeqGene on chromosome 2 | TTACCACTGGCTTGT |
| | | GCTAAAAGCCTTGAC |
| SEQ ID | GI 54112075 AY771350 Homo sapiens | GACTGCTGAGGCCAC |
| NO: 3897 | heme oxygenase | AGCAACTTAACTGGC |
| | (decycling) 2 | TAGTGTGGGTGAGGC |
| | (HMOX2) gene, complete cds | AGAAGTGTTTGAAGC |
| SEQ ID | GI 1036031265 AH007464 Homo sapiens | GGAGAAGGTGTCTGC |
| NO: 3898 | methylenetetrahydrofolate reductase | GGGAGCCGATTTCAT |
| | (MTHFR) gene, complete cds | CATCACGCAGCTTTT |
| | | CTTTGAGGCTGACAC |
| SEQ ID | GI 1036032759 AH002916 Homo sapiens | CGACATTATCAGGTT |
| NO: 3899 | ornithine aminotransferase | TGCGCCTCCGCTGGT |
| | (OAT) gene, complete cds | GATCAAGGAGGATGA |
| | | GCTTCGAGAGTCCAT |
| SEQ ID | GI 994323650 NG_011530 Homo sapiens | CACATCCCCACCTCT |
| NO: 3900 | 2′-5′-oligoadenylate synthetase 1 | CATCTGACCAATGGG |
| | (OAS1), RefSeqGene on chromosome 12 | AGACTCTGAGCAGCT |
| | | GAGTGACTTGGGTTG |
| SEQ ID | GI 747165391 NM_004590 Homo sapiens | CCGAGAAGTCTGCAC |
| NO: 3901 | C-C motif chemokine ligand 16 | CAACCCCAATGACGA |
| | (CCL16), mRNA | CTGGGTCCAAGAGTA |
| | | CATCAAGGATCCCAA |
| SEQ ID | GI 149408121 NM_024119 Homo sapiens | AAGCTCATGGACCAA |
| NO: 3902 | DExH-box helicase 58 | ATCCATGACCACCTG |
| | (DHX58), mRNA | GAGATGCCTGAGTTG |
| | | AGCCGGAAATTTGGG |
| SEQ ID | GI 221316649 NM_005232 Homo sapiens | TTTAGCCACCCGCAT |
| NO: 3903 | EPH receptor A1 | ATTCTGCATCTGGAA |
| | (EPHAl), mRNA | GGCGTCGTCACAAAG |
| | | CGAAAGCCGATCATG |
| SEQ ID | GI 334883185 NG_029065 Homo sapiens | ACCTCTAGGCATTGC |
| NO: 3904 | cAMP responsive element modulator | CTCAGACAGGAAGAA |
| | (CREM), RefSeqGene on chromosome 10 | GGGCTAGAGCCAAAA |
| | | ATTGGGGCTAGCTGA |
| SEQ ID | GI 1024846809 NG_046989 Homo sapiens | TCACAAGATGCTCTC |
| NO: 3905 | matrix metallopeptidase 14 | TGGAGCCAAGGGGGA |
| | (MMP14), RefSeqGene on chromosome 14 | GAAATGGGAATGGAG |
| | | GACCAGAGACAAAGG |
| SEQ ID | GI 1027250665 NG_047132 Homo sapiens | AACCACTCCCAGCAT |
| NO: 3906 | peroxisome proliferator- | TCCTGCATGCCCTCT |
| | activated receptor | TATCCTTCAGGTCCA |
| | gamma, coactivator-related 1 | GCTGTAGTTCCTCTG |
| | (PPRC1), RefSeqC [symbol] | |
| SEQ ID | GI 323362950 NM_138621 Homo sapiens | ACTTGTGACCCCAGC |
| NO: 3907 | BCL2 like 11 | CATGTAGTGAGGGTG |
| | (BCL2L11), transcript variant 1, mRNA | CTCTTTCTCTGTGCC |
| | | TGCTCCTTATGAGTG |
| SEQ ID | GI 33413399 NM_001984 Homo sapiens | TCCAGTGGATCCCCA |
| NO: 3908 | esterase D | AAGGATGTCTATTTT |
| | (ESD), mRNA | TGGCCACTCCATGGG |
| | | AGGTCATGGAGCTCT |
| SEQ ID | GI 32891798 AY341246 Homo sapiens | TGAAAACCACCTCAG |
| NO: 3909 | catechol-O-methyltransferase | CATAGTGTCACCTGC |
| | (COMT) gene, complete cds | TCCTCTGACACTGTC |
| | | GCTTCTCCATGGCAT |
| SEQ ID | GI 66841738 DQ075211 Homo sapiens | TTTCCCAAGGTCACA |
| NO: 3910 | c-src tyrosine kinase | CAGCAAGTTGATGGC |
| | (CSK) gene, complete cds | AGAGCTGGGTACAGG |
| | | ACTCAGAGCATCTGA |
| SEQ ID | GI 1024846791 NG_047016 Homo sapiens | CTCATCTCAGGGCTG |
| NO: 3911 | bactericidal/permeability- | TTGGAAGAAGAGAGT |
| | increasing protein | AAGGGGGTGTCTGTG |
| | (BPI), RefSeqGene on chromosome 20 | AATGTGGGGTGTGGG |
| SEQ ID | GI 388490278 NG_032761 Homo sapiens | ATGCTTTTTGCTGGA |
| NO: 3912 | TNF alpha induced protein 3 | GCGTTTTCTACCGAC |
| | (TNFAIP3), RefSeqGene on chromosome 6 | AGTGACAAGCAGGCT |
| | | TTCCTCTCTGCCAGG |
| SEQ ID | GI 222080101 NM_022121 Homo sapiens | CCACTATGGTGTATG |
| NO: 3913 | PERP, TP53 apoptosis effector | TCATTGTGAGGATGG |
| | (PERP), mRNA | GTGCAGAGAGGCTGG |
| | | GTGGGAGAACGGAAA |
| SEQ ID | GI 336285452 NM_006221 Homo sapiens | CAGACTCGAGGGCCG |
| NO: 3914 | peptidylprolyl cis/trans | AATTGTTTCTAGTTA |
| | isomerase, NIMA-interacting 1 | GGCCACGCTCCTCTG |
| | (PIN1), transcript variant 1, mRNA | TTCAGTCGCAAAGGT |
| SEQ ID | GI 406737 X75342 H. sapiens | CTCTCGCCATGGCCA |
| NO: 3915 | SHB mRNA | AGTGGCTAAACAAGT |
| | | ACTTCAGCTTGGGCA |
| | | ACAGCAAGACCAAGA |
| SEQ ID | GI 460417326 NG_008384 Homo sapiens | CCTCAATCTTAGGGG |
| NO: 3916 | cytochrome P450 family 2 | AAAGGCTGAGGCACC |
| | subfamily C member 19 | AAGCAAGGACACATG |
| | (CYP2C19), RefSeqGene on | CAGACCATTTCCAGG |
| | chromosome [symbol] | |
| SEQ ID | GI 297139781 NG_021374 Homo sapiens | AGGCCTCCCGCTTCT |
| NO: 3917 | early growth response 1 | CTCTCTCCTGCCAGA |
| | (EGR1), RefSeqGene on chromosome 5 | GTCTTTTCCTGACAT |
| | | CTCTCTGAACAACGA |
| SEQ ID | GI 908673232 NG_042289 Homo sapiens | CCCATGGCAGCTTTG |
| NO: 3918 | sodium voltage-gated | TACCTGCCCCTCACC |
| | channel alpha subunit 3 | ACTACCTGAGCCCTT |
| | (SCN3A), RefSeqGene on chromosome 2 | TTCTAGGAACTACTC |
| SEQ ID | GI 187473261 EU595874 Homo sapiens | GAGCATCAGAGGAAA |
| NO: 3919 | carboxylesterase 3 | CAACTGCTGCATTCC |
| | (brain) | AGCATATTGGCACTC |
| | (CES3) gene, complete cds | AGGGAGGGCTTCCTG |
| SEQ ID | GI 42794755 NM_016234 Homo sapiens | TGTCAACAAGGCTGA |
| NO: 3920 | acyl-CoA synthetase long | TATCGCCATGGTGAT |
| | chain family member 5 | CTGTGACACACCCCA |
| | (ACSL5), transcript variant 1, mRNA | AAAGGCATTGGTGCT |
| SEQ ID | GI 1036029324 AHO13155 Homo sapiens | GCCATAACCCCGGAG |
| NO: 3921 | chromosome 19 poliovirus | TCAGAGGGATGCCTG |
| | receptor related 2 | GGTCTTAATCTCCAC |
| | (PVRL2) gene, | TTGTCCTACCTCCTA |
| SEQ ID | GI 54112083 AY771996 Homo sapiens | GTGTGAGCAGGAGAG |
| NO: 3922 | interleukin 12 receptor, beta 1 | GCACATGACTGGACT |
| | (IL12RB1) gene, complete cds | CAGATGTTCACAGAC |
| | | TTTCCCTGGCTCCTG |
| SEQ ID | GI 440503006 JX512449 Homo sapiens | GAGACCTGAGTCTTG |
| NO: 3923 | mitogen-activated protein kinase 1 | GTCCCTGCTTCTGAT |
| | (MAPK1) gene, complete cds | GACATGTCACATGGG |
| | | CATGTGCTCGTCACT |
| SEQ ID | GI 50234988 AY675320 Homo sapiens | TGATGGAGGCCAGCA |
| NO: 3924 | RAD21 homolog | GAACAAACATAGATG |
| | (S. pombe) | AGTCAGCTATGCCTC |
| | (RAD21) gene, complete cds | CACCACCACCTCAGG |
| SEQ ID | GI 259013222 NG_012922 Homo sapiens | GCGGTTTTAGTGGTT |
| NO: 3925 | glycogen synthase kinase 3 beta | AGAAGCCGCAGGTAA |
| | (GSK3B), RefSeqGene on chromosome 3 | GGTCCTTCCCAGGCC |
| | | AGCTCAAGTTTTTCC |
| SEQ ID | GI 224809244 NG_011449 Homo sapiens | GAATCCAGTATGCAA |
| NO: 3926 | 3-hydroxy-3-methylglutaryl-CoA reductase | TGGATGTCGCACACA |
| | (HMGCR), RefSeqGene on chromosome 5 | AGAGAGAGAGACGCA |
| | | GGTGACCGGAGCCAT |
| SEQ ID | GI 300796044 NG_023367 Homo sapiens | GGCAGCCTTCCTAGG |
| NO: 3927 | platelet derived growth | TTCTTCCTGTCTTCC |
| | factor receptor beta | AAGTGCTAGCCATGC |
| | (PDGFRB), RefSeqGene on chromosome 5 | ACTTTGTGCTGAAGC |
| SEQ ID | GI 1024846788 NG_047009 Homo sapiens | TTCACCCTGGTCTGC |
| NO: 3928 | platelet and endothelial | TCTGAAGTTTGGATA |
| | cell adhesion molecule 1 | CAAGTCCTGACCCAT |
| | (PECAM1), RefSeqGene on chromosome | GGGCATGGTTCAGGA |
| SEQ ID | GI 242247668 NM_032962 Homo sapiens | TGAGTGACCCAGAAG |
| NO: 3929 | C-C motif chemokine ligand 14 | GGGTGGCGAAGGCAC |
| | (CCL14), transcript variant 2, mRNA | AGCTCAGAGACATAA |
| | | AGAGAAGATGCCAAG |
| SEQ ID | GI 325652112 NM_020997 Homo sapiens | TGTTCGAGGGTACCA |
| NO: 3930 | left-right determination factor 1 | GGAGAGCTGGCGATG |
| | (LEFTY1), mRNA | ACTGAACTGCTGATG |
| | | GACAAATGCTCTGTG |
| SEQ ID | GI 385137136 NM_012340 Homo sapiens | TGGAAGCCACGGTGG |
| NO: 3931 | nuclear factor of activated T-cells 2 | ATAAGGACAAGAGCC |
| | (NFATC2), transcript variant 1, mRNA | AGCCCAACATGCTTT |
| | | TTGTTGAGATCCCTG |
| SEQ ID | GI 117606467 EF064708 Homo sapiens | GTGCCCTGCATCTTG |
| NO: 3932 | tumor necrosis factor | AGGCTTGGGTCTGTC |
| | (ligand) family, member 10 | CAATAATTTTAAGCC |
| | (TNFSF10) gene, complete cds | CCTGGGCTCTGCTAC |
| SEQ ID | GI 359718928 NG_031876 Homo sapiens | CTCTTCACAGGTCAG |
| NO: 3933 | biliverdin reductase A | TGCTACGTGGGATCA |
| | (BLVRA), RefSeqGene on chromosome 7 | CAGGTCACATGTGAG |
| | | GTCCAAAGACCAGCC |
| SEQ ID | GI 226491119 NG_011810 Homo sapiens | GTGAGAACCATAGGA |
| NO: 3934 | 1,4-alpha-glucan branching enzyme 1 | ATACACCCACTGCGG |
| | (GBE1), RefSeqGene on chromosome 3 | TGCCCAGGTGTGACC |
| | | AGGTGCAGATATTCT |
| SEQ ID | GI 1145587777 KX533498 Homo sapiens | CGATGGGGGTCTCAT |
| NO: 3935 | macrophage receptor | TGGCCCAAAAGGGGA |
| | (MARCO) mRNA, complete cds | AACTGGAACTAAGGG |
| | | AGAGAAAGGAGACCT |
| SEQ ID | GI 41324131 AY527817 Homo sapiens | ATGATCAGAAAGGGG |
| NO: 3936 | arachidonate 12-lipoxygenase | CCTGGCCATTTGACA |
| | (ALOX12) gene, complete cds | GGTGGTCAGTTTACA |
| | | ATGCCTGTGATGCCA |
| SEQ ID | GI 922304389 NG_042830 Homo sapiens | CAGTACCTGAAGAGG |
| NO: 3937 | acyl-CoA dehydrogenase family member 11 | ATGATGCCCTCATAC |
| | (ACAD11), RefSeqGene on chromosome 3 | TGCCAGGTGGGATGA |
| | | AAGTCCAGGCATCCC |
| SEQ ID | GI 350606343 NG_030328 Homo sapiens | CACCCGTTGGTTTGA |
| NO: 3938 | neuropilin 1 | AGAAGGGCGGGCTCT |
| | (NRP1), RefSeqGene on chromosome 10 | CACATAAGCAACCAG |
| | | GTAAGGAAAGGAAGC |
| SEQ ID | GI 340007370 NG_029431 Homo sapiens | CCACTTTCTTCCTCA |
| NO: 3939 | ras-related C3 botulinum toxin substrate 1 | GCTGCCACATCCAAA |
| | (rho family, small GTP binding protein Rael) | CCATGGGTAAGCGAT |
| | (RAC [symbol] | GCTTGATCTGCCTCC |
| SEQ ID | GI 264681561 NM_018441 Homo sapiens | TTCTAAGGGATGGCA |
| NO: 3940 | peroxisomal trans-2-enoyl-CoA reductase | CGCTGTGCTTGAGAC |
| | (PECR), mRNA | CAACCTGACGGGTAC |
| | | CTTCTACATGTGCAA |
| SEQ ID | GI 520262262 NM_002872 Homo sapiens | CAACCAGCCCCTGCC |
| NO: 3941 | ras-related C3 botulinum toxin substrate 2 | ATTTCTTAAGACTTT |
| | (rho family, small GTP binding protein Rac2) | CTGCTGCACTCACAG |
| | (RA+z,899 | GATCCTGAGCTGCAC |
| SEQ ID | GI 229608964 NM_014600 Homo sapiens | TCCCAAGAAACCCTT |
| NO: 3942 | EH domain containing 3 | CAGGAAACTCAACGC |
| | (EHD3), mRNA | CTTTGGCAACGCCTT |
| | | CTTGAACAGGTTCGT |
| SEQ ID | GI 345842406 NM_005849 Homo sapiens | GGACTACACTCATGA |
| NO: 3943 | immunoglobulin superfamily member 6 | GGCCGTCACCATAAA |
| | (IGSF6), mRNA | GTGTACCTTCTCCGC |
| | | AACCGGATGCCCTTC |
| SEQ ID | GI 289546502 NM_001172567 Homo sapiens | CCAGGCTGGAGCAAG |
| NO: 3944 | myeloid differentiation primary response 88 | GTACCTTTTCTTAGG |
| | (MYD88), transcript variant 1, mRNA | ATCTTGGGAGGGAAT |
| | | GGATGCCCCTCTCTG |
| SEQ ID | GI 22027540 NM_005707 Homo sapiens | TAGGAGGCTGCTAGT |
| NO: 3945 | programmed cell death 7 | TAGGAAGATGGCAGT |
| | (PDCD7), mRNA | GGCTGTAGGCTGGGT |
| | | TGCCAGAAAAGATGG |
| SEQ ID | GI 94557300 NM_006251 Homo sapiens | GGTAGCCTGGGGCTA |
| NO: 3946 | protein kinase AMP-activated | GATGTGGAACTCCAT |
| | catalytic subunit alpha 1 | GGATCTGTGTTCTGA |
| | (PRKAA1), transcript variant 1, mRNA | CTGGCACCTTTGGAA |
| SEQ ID | GI 36030972 NM_181713 Homo sapiens | GTCTACGAAGCCTGT |
| NO: 3947 | UBX domain protein 2A | GTTCCAGCCCTTTTC |
| | (UBXN2A), mRNA | AGGACAGGGTCACAG |
| | | ACTAGGAAGTGCCAC |
| SEQ ID | GI 14278706 AF378903 Homo sapiens | GAGTTCCCAGAAGCT |
| NO: 3948 | protein C | TGTCTCTGCTTTTCT |
| | (PROC) gene, complete cds | AGGCAGCTGCCACAG |
| | | CCTGTCACAAACAGC |
| SEQ ID | GI 514052655 NM_001278549 Homo sapiens | AGTGCCCTCATTTAA |
| NO: 3949 | pyruvate dehydrogenase kinase 1 | GAGGCCAAGATCCTG |
| | (PDK1), transcript variant 1, mRNA | ATTCAGAGGAGGCAT |
| | | CCTTTGCCCAGAGCT |
| SEQ ID | GI 46241839 AY588246 Homo sapiens | GTTCACCTGCTTGAG |
| NO: 3950 | epidermal growth factor receptor | GTCAGTTCCTTGAGC |
| | (erythroblastic leukemia viral | GTCTTCCACTTGCCA |
| | (v-erb-b) oncogene homolog, a [symbol] | TGTTCACCACAGTGC |
| SEQ ID | GI 282394040 NG_016265 Homo sapiens | AGTGTCACCCTTCTG |
| NO: 3951 | hephaestin | CTCGTTGTTCTGGCT |
| | (HEPH), RefSeqGene on chromosome X | CTTGGTGGAGTGGTT |
| | | TGGTACCAACATCGA |
| SEQ ID | GI 254911080 NG_012514 Homo sapiens | CACACCTGGATGATT |
| NO: 3952 | interleukin 13 receptor subunit alpha 2 | GCCATTGCTTCCAAA |
| | (IL13RA2), RefSeqGene on chromosome X | ATGACGGTCGCTCTG |
| | | CTCCCAATCTCTCCC |
| SEQ ID | GI 1212890194 NG_032955 Homo sapiens | AAATGTGATGGGGTT |
| NO: 3953 | mitogen-activated protein | CTGCAGTTAGATGAG |
| | kinase 8 interacting protein 2 | TCTGAAAGTGGGGCT |
| | (MAPK8IP2), RefSeqGene on chror [symbol] | GGGGCTGCTCTGAGA |
| SEQ ID | GI 22538799 NM_005408 Homo sapiens | TGTCTCTAAGCCCCC |
| NO: 3954 | C-C motif chemokine ligand 13 | TTCCCTTCCACTATG |
| | (CCL13), mRNA | AGCTGCTGGCAGTGG |
| | | GTTTGTATTCGGTTC |
| SEQ ID | GI 54781368 AY791292 Homo sapiens | CCCCACCCCTTTTCC |
| NO: 3955 | cyclin GI | CCCTTTCCAGAGTTT |
| | (CCNG1) gene, complete cds | TGTTCTGTGGCATTG |
| | | CCTTGGGTAAGTACA |
| SEQ ID | GI 1036032447 AH002989 Homo sapiens | TGCCTCTCAGTGTCT |
| NO: 3956 | pp60 c-src-1 protein | GACTTCGACAACGCC |
| | (SRC) gene, complete cds | AAGGGCCTCAACGTG |
| | | AAGCACTACAAGATC |
| SEQ ID | GI 193788644 NG_008170 Homo sapiens | TTAGTGGACTGGAAA |
| NO: 3957 | branched chain amino acid transaminase 1 | CCAAGTGAACCTGGA |
| | (BCAT1), RefSeqGene on chromosome 12 | AACCACAAGCAGCTG |
| | | TGGCAAATCCTGCCA |
| SEQ ID | GI 225735562 NG_011713 Homo sapiens | GGAAGAGAGCCAAAT |
| NO: 3958 | insulin like growth factor 1 | GCAAACCAGACGATA |
| | (IGF1), RefSeqGene on chromosome 12 | TCCCAGCTGGTTTGA |
| | | ATGGCCTCCACCGTG |
| SEQ ID | GI 699969198 NG_041780 Homo sapiens | GCTGTTGGCTGGAGG |
| NO: 3959 | NLR family CARD domain containing 4 | CCTCACTTCCTCATC |
| | (NLRC4), RefSeqGene on chromosome 2 | ATTAGACCTCTTCAC |
| | | AGGCTGTGAGTGTCC |
| SEQ ID | GI 306922359 NG_027701 Homo sapiens | ATGAAGGGACTCCTG |
| NO: 3960 | retinoic acid receptor alpha | CAGTCCATGAGGAAG |
| | (RARA), RefSeqGene on chromosome 17 | AGGACAGAAGTGAGC |
| | | GCCCAACCAAAATGA |
| SEQ ID | GI 874507494 NG_042223 Homo sapiens | AAAAGCCCCTGTTCC |
| NO: 3961 | tubulin beta 2A class Ila | AAGGACATGGGAGGA |
| | (TUBB2A), RefSeqGene on chromosome 6 | GGACAAGGCCATCCT |
| | | TAGTGACATTCAGGA |
| SEQ ID | GI 256600243 NG_012682 Homo sapiens | CTACAGGGCGGCAAC |
| NO: 3962 | versican | CAGCAGGTACACACT |
| | (VCAN), RefSeqGene on chromosome 5 | GAATTTTGAGGCTGC |
| | | TCAGAAGGCTTGTTT |
| SEQ ID | GI 70995210 NM_001398 Homo sapiens | TGTCCAGCTCAACCG |
| NO: 3963 | enoyl-CoA hydratase 1 | GCCCAACAAGAGGAA |
| | (ECH1), mRNA | TGCCATGAACAAGGT |
| | | CTTCTGGAGAGAGAT |
| SEQ ID | GI 372466583 NM_005239 Homo sapiens | GGACCCAGGAAAGGC |
| NO: 3964 | ETS proto-oncogene 2, transcription factor | AGGATTGAAAATGTC |
| | (ETS2), transcript variant 1, mRNA | CAGGAAAGTGGCCAA |
| | | GAAGCAGTGGCCTTA |
| SEQ ID | GI 1003088850 NM_007368 Homo sapiens | CCTCTCTACAGCATT |
| NO: 3965 | RAS p21 protein activator 3 | CCCATCGAGAACATC |
| | (RASA3), transcript variant 1, mRNA | CTGGCAGTGGAGAAG |
| | | CTGGAGGAGGAGTCT |
| SEQ ID | GI 440502998 JX512445 Homo sapiens | TAAGGGAGCTATGGG |
| NO: 3966 | integrin, alpha V | ATGTGTGGAGGAGTG |
| | (ITGAV) gene, complete cds | TTTGCCACAGAGGAA |
| | | CTGGCAGTTTCAGCA |
| SEQ ID | GI 70995210 NM_001398 Homo sapiens | CGTGAAGTCGGTCCA |
| NO: 3967 | enoyl-CoA hydratase 1 | GGCCACGACTGAGAA |
| | (ECH1), mRNA | CAAGGAACTGAAAAC |
| | | CGTCACCTTCTCCAA |
| SEQ ID | GI 217035112 NM_152672 Homo sapiens | TGCCATCTTCCTGGA |
| NO: 3968 | solute carrier family 51 alpha subunit | GGATGCCGTCTACCT |
| | (SLC51A), mRNA | GTACAAGAACACCCT |
| | | TTGCCCCATCAAGAG |
| SEQ ID | GI 1049010968 AH002726 Homo sapiens | TCCGGGACTGGGATC |
| NO: 3969 | c-fgr peptide | AGACCAGAGGCGATC |
| | (FGR) gene, complete cds | ATGTGAAGCATTACA |
| | | AGATCCGCAAACTGG |
| SEQ ID | GI 225735573 NG_011718 Homo sapiens | TAAACGTGGAACCCC |
| NO: 3970 | alcohol dehydrogenase 1C | AGAGTGGCTTGTGTC |
| | (class 1), gamma polypeptide | CTGGTGGACCTAAAG |
| | (ADHIC), RefSeqGene on chromoso [symbol] | TGGTGCACATGATCT |
| SEQ ID | GI 237874189 NG_012169 Homo sapiens | GAAAGACTCCCTTGC |
| NO: 3971 | angiopoietin like 4 | CTGGCCTCAGCGGAT |
| | (ANGPTL4), RefSeqGene on chromosome 19 | GGAGATTTGGAAGGA |
| | | TGGATGAGTGGATGA |
| SEQ ID | GI 582015216 NG_016002 Homo sapiens | TGCTGTTGAGCTTGG |
| NO: 3972 | G protein subunit alpha i2 | CCAGAGTACCCAAGT |
| | (GNAI2), RefSeqGene on chromosome 3 | TTGACCTCTGCACAG |
| | | ACCTTACTACCCACC |
| SEQ ID | GI 1139349906 NG_052802 Homo sapiens | TGCCAATGTCCTGAA |
| NO: 3973 | NLR family pyrin domain containing 4 | GGACGAAGGACTGAA |
| | (NLRP4), RefSeqGene on chromosome 19 | AACTCTCTGCGAGGC |
| | | CTTGAAACATCCGGA |
| SEQ ID | GI 1152003149 NG_016599 Homo sapiens | TGCTGCTTCATGCCT |
| NO: 3974 | phosphorylase kinase regulatory subunit alpha 1 | GACAATATTCTGATC |
| | (PHKA1), RefSeqGene on chromosome X | TTTGTCAGTGTCTCA |
| | | GCCCTCCCCACCCCT |
| SEQ ID | GI 345478701 NG_029901 Homo sapiens | GAAGGGCTGTACCCC |
| NO: 3975 | peroxiredoxin 2 | ATGACACAGCTAGAG |
| | (PRDX2), RefSeqGene on chromosome 19 | GAGGATGGGGACTCT |
| | | TAACACAGGCTCTCT |
| SEQ ID | GI 167614484 NM_006111 Homo sapiens | ACGAATTAAGGCGTC |
| NO: 3976 | acetyl-CoA acyltransferase 2 | GAGGTGGAAAATATG |
| | (ACAA2), mRNA | CCGTTGGATCAGCTT |
| | | GCATTGGAGGTGGCC |
| SEQ ID | GI 156523973 NM_001296 Homo sapiens | ATGAAGTGGCCTTCA |
| NO: 3977 | atypical chemokine receptor 2 | TGCTCTGCAGGAAGG |
| | (ACKR2), mRNA | ATGCAGTGGTGTCCT |
| | | TTGGCAAAGTCTTCC |
| SEQ ID | GI 68215223 NM_003965 Homo sapiens | GCACTGAATTTGTCT |
| NO: 3978 | C-C motif chemokine receptor like 2 | CAGGCACCGTGCAAG |
| | (CCRL2), transcript variant 1, mRNA | GCTCTTTACAAACGT |
| | | GAGCTCCTTCGCCTC |
| SEQ ID | GI 156523967 NM_001618 Homo sapiens | GCAAAGGCCAGGATG |
| NO: 3979 | poly(ADP-ribose) polymerase 1 | GAATTGGTAGCAAGG |
| | (PARP1), mRNA | CAGAGAAGACTCTGG |
| | | GTGACTTTGCAGCAG |
| SEQ ID | GI 18765742 NM_003098 Homo sapiens | GTGGGGGATGCCATC |
| NO: 3980 | syntrophin alpha 1 | CTGTCTGTGAATGGG |
| | (SNTA1), mRNA | GAAGACTTGTCCTCT |
| | | GCTACCCATGATGAG |
| SEQ ID | GI 209977025 NG_008836 Homo sapiens | GGAACTTCAAGGATC |
| NO: 3981 | angiotensinogen | CATCATGGGATGCAG |
| | (AGT), RefSeqGene on chromosome 1 | GACAGTAGGCTCCAC |
| | | CCCTCTTCCTGCTTT |
| SEQ ID | GI 260593639 NG_013007 Homo sapiens | CCTGGGACCACCAGT |
| NO: 3982 | C-reactive protein | AGCCATCTTGTTTGC |
| | (CRP), RefSeqGene on chromosome 1 | CACATGGAGAGAGAC |
| | | TGTGAGGACAGAAGC |
| SEQ ID | GI 212549728 NG_008977 Homo sapiens | ACGTGGAACTTAGTG |
| NO: 3983 | methylmalonyl-CoA epimerase | GTCTCCCTGAGTTGC |
| | (MCEE), RefSeqGene on chromosome 2 | GGAGATAGGAGTTGG |
| | | AAGTCTGAGGAGGCC |
| SEQ ID | GI 209977032 NG_008779 Homo sapiens | AGAAAGCAGCCCTCT |
| NO: 3984 | paraoxonase 1 | ACCTCCGAAAAACAG |
| | (PON1), RefSeqGene on chromosome 7 | CAAGACGTTGCTTTC |
| | | CTGCCATAAACGCCC |
| SEQ ID | GI 194239686 NM_004591 Homo sapiens | AGCCCAAGAACAGAA |
| NO: 3985 | C-C motif chemokine ligand 20 | AGAACCTTGCTGGGG |
| | (CCL20), transcript variant 1, mRNA | TTGGAGGTTTCACTT |
| | | GCACATCATGGAGGG |
| SEQ ID | GI 237874277 NG_012191 Homo sapiens | CTTTTGCACTTGCTA |
| NO: 3986 | BCL2 associated X, apoptosis regulator | ATTCCTTCTGCGCTG |
| | (BAX), RefSeqGene on chromosome 19 | GGGAGAGCTCAAACC |
| | | CTGCCCGAAACTTCT |
| SEQ ID | GI 341865570 NG_029565 Homo sapiens | ATGGCTGCTGAGTTC |
| NO: 3987 | interleukin 24 | CAGAAGGCAAAGACA |
| | (IL24), RefSeqGene on chromosome 1 | CAGAGATGGCTCAGC |
| | | TCCATCAAGTGGAGT |
| SEQ ID | GI 153082754 NM_001547 Homo sapiens | GCTGCCATCTGCCAC |
| NO: 3988 | interferon induced protein | CTGCTACCATTGACC |
| | with tetratricopeptide repeats 2 | TACGTGAACACAGAC |
| | (IFIT2), mRNA | ATTCTGTCTCCACCT |
| SEQ ID | GI 169234615 NM_002514 Homo sapiens | GTTTGGTAAAAGCGA |
| NO: 3989 | nephroblastoma overexpressed | GAGGGGAAAGCCTGA |
| | (NOV), mRNA | GCATGCAGAGTGTGC |
| | | AGAGCACGAGCTTTT |
| SEQ ID | GI 197100280 NG_008374 Homo sapiens | CAAAGATCTCAGCAG |
| NO: 3990 | cytochrome P450 family | CCAGCTCCTGGTAGC |
| | 11 subfamily B member 2 | TGATGAGGATCAGAG |
| | (CYP11B2), RefSeqGene on chromosome [symbol] | CGTTTGTTCCCCCAT |
| SEQ ID | GI 168480137 NG_007294 Homo sapiens | ACACCACCCTGTTAA |
| NO: 3991 | hydroxyacyl-CoA dehydrogenase/ | GCCCAGGTGGTGAAG |
| | 3-ketoacyl-CoA thiolase/enoyl-CoA hydratase | AGCTGGTTAGGTTGG |
| | (trifunctiona [symbol] | TCATTTGCAAAACCG |
| SEQ ID | GI 282394040 NG_016265 Homo sapiens | ATGTGCCCAGCCTGC |
| NO: 3992 | hephaestin | CTGGAGAAAAGTGTC |
| | (HEPH), RefSeqGene on chromosome X | TGCTCCTAGCCAAGA |
| | | TCTCCTCATCACAAA |
| SEQ ID | GI 237858735 NG_012106 Homo sapiens | TTCTCTGCCCCCTGT |
| NO: 3993 | 8-oxoguanine DNA glycosylase | TGCTGCTACTGTGGT |
| | (OGG1), RefSeqGene on chromosome 3 | TCCAAACCCTTACCA |
| | | TCTCCTGCCTGTATC |
| SEQ ID | GI 498917180 NM_032375 Homo sapiens | CAGAGACCTCCGGGG |
| NO: 3994 | AKT1 substrate 1 | AGCAGCTGGGCATTA |
| | (AKT1S1), transcript variant 1, mRNA | GTGATAATGGAGGGC |
| | | TCTTTGTGATGGATG |
| SEQ ID | GI 156142193 NM_005880 Homo sapiens | AAAGCAGCAGCCATC |
| NO: 3995 | DnaJ heat shock protein family | ATGGACCTGGAGTGC |
| | (Hsp40) member A2 | AGTGTGCCCATCAGT |
| | (DNAJA2), mRNA | AAACTCTGCAAACAA |
| SEQ ID | GI 8248053 AF199441 Homo sapiens | CAGGAAAGCAGGAAG |
| NO: 3996 | gastrointestinal glutathione peroxidase | GCTGAAGCATCACTG |
| | (GPX2) gene, complete cds | GCTGTGACAAACCTG |
| | | AGCCCAAACTGTTCA |
| SEQ ID | GI 297139734 NG_021330 Homo sapiens | TCACACATGTAACAG |
| NO: 3997 | serum amyloid A1 | GTGCAGAGCCAAGAC |
| | (SAA1), RefSeqGene on chromosome 11 | TGCAGGCTGGTTTGT |
| | | CTGACTCCAGAACCT |
| SEQ ID | GI 61744441 NM_003262 Homo sapiens | TGCCAGTCCCTGCAC |
| NO: 3998 | SEC62 homolog, preprotein | TCTTCCTTTCTGGCC |
| | translocation factor | AAGGATGATGTCAGT |
| | (SEC62), mRNA | TCTGCTGCTACTAGC |
| SEQ ID | GI 190194409 NG_008004 Homo sapiens | TAAAAGGCGCAACCA |
| NO: 3999 | activin A receptor type 1 | AGAACGCCTCAATCC |
| | (ACVR1), RefSeqGene on chromosome 2 | CCGAGACGTGGAGTA |
| | | TGGCACTATCGAAGG |
| SEQ ID | GI 368711279 NG_031983 Homo sapiens | CCACACCCCAACCTA |
| NO: 4000 | heat shock protein family B | AGAGTGCACATCTGT |
| | (small) member 6 | GTGCCTAGCCTGATC |
| | (HSPB6), RefSeqGene on chromosome 19 | CAGGATTCACGTGAG |
| SEQ ID | GI 189458878 NM_139166 Homo sapiens | GCGTGCCAGGAAACA |
| NO: 4001 | actin binding Rho activating protein | TGGACTGGTAGACTT |
| | (ABRA), mRNA | TGAAGGAGAGATGCT |
| | | ATGGCAAGGCCGAGA |
| SEQ ID | GI 584458490 NG_012645 Homo sapiens | GGAGACCCTTGCCAA |
| NO: 4002 | FK506 binding protein 5 | CATCACAGCTAGGGT |
| | (FKBP5), RefSeqGene on chromosome 6 | CTTACTTGCCTTCCG |
| | | TGTGAGTAAGGCCAG |
| SEQ ID | GI 1135567463 NG_052785 Homo sapiens | AGGACGACCACACGC |
| NO: 4003 | galanin and GMAP prepropeptide | CGCTTCCTGTAGCAT |
| | (GAL), RefSeqGene on chromosome 11 | GTGTCGTGGTTGTAA |
| | | TTCTCTCATGTGCAT |
| SEQ ID | GI 1143077144 NG_052854 Homo sapiens | GGTCTCTCTCCAGAC |
| NO: 4004 | gem nuclear organelle associated protein 5 | CTTGCCCAAATTCCA |
| | (GEMIN5), RefSeqGene on chromosome 5 | GTGTCTGGGTAAGGG |
| | | CTGGTCACAGAACAC |
| SEQ ID | GI 308080224 NG_027757 Homo sapiens | TAGTCAATAGGCGGT |
| NO: 4005 | transforming growth factor beta receptor 3 | CACTGGTGACCATAA |
| | (TGFBR3), RefSeqGene on chromosome 1 | CAGGATGCATTTCAG |
| | | GGCAGTGGTTGCGGA |
| SEQ ID | GI 63477961 NM_000911 Homo sapiens | TACAACTGAGTCCTT |
| NO: 4006 | opioid receptor delta 1 | AAACAGGGCATCTCC |
| | (OPRD1), mRNA | AGGAAGGCGGGGCTT |
| | | CAACCTTGAGACAGC |
| SEQ ID | GI 67515408 DQ080435 Homo sapiens | GTTCCCCCAAACTGC |
| NO: 4007 | calcitonin/calcitonin- | AATTGGGGTTGGAGC |
| | related polypeptide, alpha | ACCTGGAAAGAAAAG |
| | (CALCA) gene, complete cds | GGATATGTCCAGCGA |
| SEQ ID | GI 21655314 AY124010 Homo sapiens | TTTGGCTGTGGCCTC |
| NO: 4008 | interleukin 1 receptor, type II | TTCCAGTAATTGCTC |
| | (IL1R2) gene, complete cds | TGCTGGGAAAGTCAT |
| | | TGGCCAAGGATGCTC |
| SEQ ID | GI 1024336747 NG_046907 Homo sapiens | TTCTTGTTCTGGCCC |
| NO: 4009 | acetyl-CoA carboxylase beta | TCCACAGTGGTGAAG |
| | (ACACB), RefSeqGene on chromosome 12 | AAGGGAGTTTTACGT |
| | | AGCCCAGGCACTCAC |
| SEQ ID | GI 197245401 NG_008401 Homo sapiens | GTCATCAAAGACTGC |
| NO: 4010 | glial fibrillary acidic protein | ACAGCTACTGAGTGG |
| | (GFAP), RefSeqGene on chromosome 17 | CCAAGGCTTTGCTCC |
| | | CTCTCCTTGAGGACA |
| SEQ ID | GI 1025608653 NG_047027 Homo sapiens | TGTGCCATAAGTGGA |
| NO: 4011 | Jun proto-oncogene, AP-1 | TGCTCTGACAGCCAT |
| | transcription factor subunit | GATGTTCCGAGTTCC |
| | (JUN), RefSeqGene on chromosome [symbol] | TTCCCCACAGTCAGG |
| SEQ ID | GI 170932506 NG_007486 Homo sapiens | TTGCCTCTGGTCAGA |
| NO: 4012 | protein kinase AMP-activated | GGTACAGTAGCGTGA |
| | non-catalytic subunit gamma 2 | GGATTCGTAGTGTGG |
| | (PRKAG2), RefSeqGene on chr [symbol] | CTGGCGTTCTCATCA |
| SEQ ID | GI 974987490 NG_016453 Homo sapiens | CTTCTTCAGACAGCC |
| NO: 4013 | transient receptor potential | AGGCCTGCGACGACT |
| | cation channel subfamily M member 1 | GAGTATGTAGGTGTA |
| | (TRPM1), RefSeqGene or [symbol] | TGGGATGGAGTCCGT |
| SEQ ID | GI 557878734 NM_001995 Homo sapiens | TTCTTCTCCAGTCCC |
| NO: 4014 | acyl-CoA synthetase long | CTGTGGTTTCTGTTG |
| | chain family member 1 | GAGCATAAGGTTTCC |
| | (ACSL1), transcript variant 1, mRNA | AGTAAGCGGGAGGGC |
| SEQ ID | GI 197304706 NM_032350 Homo sapiens | CGGATCACGTACCTG |
| NO: 4015 | chromosome 7 open reading frame 50 | TGCAGAAACCGCCTC |
| | (C7orf50), transcript variant 1, mRNA | TGTGGCTGCATTTGA |
| | | AATAAAACCCGACCC |
| SEQ ID | GI 31343534 NM_178818 Homo sapiens | CACCAAGACACAGAC |
| NO: 4016 | CKLF like MARVEL transmembrane | ACGTACTCTTCACCG |
| | domain containing 4 | GCTCCTTTTGCTTCT |
| | (CMTM4), transcript variant 1, mRNA [symbol] | CCTTCCTGCAGGACG |
| SEQ ID | GI 115298685 NM_006099 Homo sapiens | CAGCACTATGGCCCC |
| NO: 4017 | protein inhibitor of activated STAT 3 | TCTGTCATCACCTCA |
| | (PIAS3), mRNA | CTAGATGAACAGGAT |
| | | GCCCTTGGCCACTTC |
| SEQ ID | GI 40288424 AY506357 Homo sapiens | GTGCAGTGACACGGC |
| NO: 4018 | truncated epidermal growth factor | ACAGATCCATGGAGT |
| | (beta-urogastrone) | GTGGAATGTGTGGAT |
| | (EGF) gene, complete cds | GGCACTTTGGGATTC |
| SEQ ID | GI 47777672 AY623118 Homo sapiens | CTCACTCATCCTCTT |
| NO: 4019 | hexokinase 2 | TCCTGCTCCCCCACA |
| | (HK2) gene, complete cds | GCTTTTCTGTTGCTT |
| | | CAGTGGCCTGACTCT |
| SEQ ID | GI 379991110 NG_032143 Homo sapiens | TGATGGTCAGGGGTG |
| NO: 4020 | aconitase 2 | CTGTTTCAGAATGTG |
| | (ACO2), RefSeqGene on chromosome 22 | GCCTGTTCTTACTGC |
| | | TGGGTGACTCGAACA |
| SEQ ID | GI 588480539 NG_033255 Homo sapiens | TCACAGAAACATGAA |
| NO: 4021 | ras homolog family member H | CTACCAGCCTTGGAG |
| | (RHOH), RefSeqGene on chromosome 4 | GGGCCCTGAGAGCCC |
| | | ATCTAGTTCAATGCC |
| SEQ ID | GI 299758401 NM_000143 Homo sapiens | CTGCTGGGGGATGCT |
| NO: 4022 | fumarate hydratase | TCAGTTTCCTTTACA |
| | (FH), mRNA | GAAAACTGCGTGGTG |
| | | GGAATCCAGGCCAAT |
| SEQ ID | GI 567316248 NM_022128 Homo sapiens | GGTCTGCCAGCTCGA |
| NO: 4023 | ribokinase | AATAACTCCAGCAAC |
| | (RBKS), transcript variant 1, mRNA | TTCTTTGGAAGCCCT |
| | | AACAATGGCCCGCAG |
| SEQ ID | GI 48928023 NM_003225 Homo sapiens | ACACGCTTCTGCAGC |
| NO: 4024 | trefoil factor 1 | TGTGCCTCGGCTCAC |
| | (TFF1), mRNA | AACACAGATTGACTG |
| | | CTCTGACTTTGACTA |
| SEQ ID | GI 1024846747 NG_047015 Homo sapiens | ACCTGAGAAGTGGCA |
| NO: 4025 | bone gamma-carboxyglutamate protein | GGTGGAGGACCCTTC |
| | (BGLAP), RefSeqGene on chromosome 1 | CAGTGATGCTCTCGT |
| | | TATAGGAAGACAGGG |
| SEQ ID | GI 313569850 NM_144601 Homo sapiens | AGTTCCTGCTGGCCT |
| NO: 4026 | CKLF like MARVEL transmembrane | TGTACTTCCTCTTTG |
| | domain containing 3 | CTGATGCCATGCAGC |
| | (CMTM3), transcript variant 1, mRN [symbol] | TGAATGACAAGTGGC |
| SEQ ID | GI 89142743 NM_023068 Homo sapiens | TCCCTCAAAGGAAGC |
| NO: 4027 | sialic acid binding ig like lectin 1 | CCTGCCCTTTCACAT |
| | (SIGLEC1), mRNA | CCTTCTCCTCGAAAG |
| | | TCACCCTGGACTTTG |
| SEQ ID | GI 88698227 DQ400449 Homo sapiens | CCTGAGCCTAGGCTT |
| NO: 4028 | complement component 5 | GGTGCTGGCACACCA |
| | (C5) gene, complete cds | TCACTTCCACACATT |
| | | CTTTTGTCCAAAGCA |
| SEQ ID | GI 42416963 AY534685 Homo sapiens | GGTACTGGGACATCA |
| NO: 4029 | insulin-like growth factor | AGACATGACTGGGAA |
| | binding protein 5 | GAGGGGGTGAGAGTC |
| | (IGFBP5) gene, complete cds | CAGAAGGGGTACCAG |
| SEQ ID | GI 226509828 NG_011801 Homo sapiens | GTGTTTTTAAGCATA |
| NO: 4030 | carnitine palmitoyltransferase 1A | AGGCCACTGATCCAG |
| | (CPT1A), RefSeqGene on chromosome 11 | GCCAGAACCCAGGGT |
| | | GGGATTTGCGCTCAT |
| SEQ ID | GI 189339243 NG_007969 Homo sapiens | AAGAGCTGGGAGTAT |
| NO: 4031 | cytochrome P450 family 7 | TGGAATCCAGGTTGC |
| | subfamily A member 1 | TCCAACTCCACAGCC |
| | (CYP7A1), RefSeqGene on chromosome 8 | AGCACTCTTCCCTTT |
| SEQ ID | GI 514825583 NG_033271 Homo sapiens | CACATTGCCGCACCT |
| NO: 4032 | Kruppel like factor 10 | TTCAAAGAGGAAGAA |
| | (KLF10), RefSeqGene on chromosome 8 | AAGAGCCCAGTATCT |
| | | GCCCCCAAACTCCCC |
| SEQ ID | GI 806904734 NG_008957 Homo sapiens | GACGTTCCAGCGGTA |
| NO: 4033 | monoamine oxidase A | GAAATCACCCACACC |
| | (MAOA), RefSeqGene on chromosome X | TTCTGGGAAAGGAAC |
| | | CTGCCCTCTGTTTCT |
| SEQ ID | GI 343403834 NG_029695 Homo sapiens | ACACTGTCTGTGGGT |
| NO: 4034 | prefoldin subunit 5 | GCTGTGCCCTGTTGG |
| | (PFDN5), RefSeqGene on chromosome 12 | AACTCTCTTTAACGT |
| | | CTTACGTTGGAGCCG |
| SEQ ID | GI 156104863 NM_003501 Homo sapiens | CTCAGTGTCGAAGAC |
| NO: 4035 | acyl-CoA oxidase 3, pristanoyl | ATGTTCAAGAGCCCT |
| | (ACOX3), transcript variant 1, mRNA | CTGAAGGTCCCCGCC |
| | | TTGATTCAGTGCCTG |
| SEQ ID | GI 61743979 NM_017564 Homo sapiens | CTTACCAAACCTGCT |
| NO: 4036 | stabilin 2 | CATGCGGCTGGAACA |
| | (STAB2), mRNA | GATGCCTGACTATTC |
| | | CATCTTCCGGGGCTA |
| SEQ ID | GI 149192859 NM_012192 Homo sapiens | CAAAAAACCACAGGG |
| NO: 4037 | translocase of inner | CTTTTGGGCACTGCC |
| | mitochondrial membrane 10B | TCCTTGGGAAGTTAG |
| | (TIMM10B), mRNA | TGGCCACAGAAGAGA |
| SEQ ID | GI 116805346 NM_014607 Homo sapiens | AATGGTGACAGTGTT |
| NO: 4038 | UBX domain protein 4 | GGCAAGGAAACAGCA |
| | (UBXN4), mRNA | ACACAGGCTGCGCTG |
| | | TTGGTAGGAGTGAAA |
| SEQ ID | GI 163954975 NG_007078 Homo sapiens | ACTCAGCATAGGGGC |
| NO: 4039 | electron transfer | TGAGTGCCAAGGTAA |
| | flavoprotein dehydrogenase | GACTTCCCCCTCACC |
| | (ETFDH), RefSeqGene on chromosome 4 | CTCTGAAGGTTCATA |
| SEQ ID | GI 224809340 NG_011507 Homo sapiens | AGACACCTCACACTG |
| NO: 4040 | glutamate ionotropic receptor | AAAAGTCTGAAAGCA |
| | NMDAtype subunit 1 | GCGGTCAAACGCTAA |
| | (GRIN1), RefSeqGene on chromosome | TGGCCAAAAGGCCCC |
| SEQ ID | GI 260763977 NG_013058 Homo sapiens | CTCAGCTTCACTGGT |
| NO: 4041 | 5-hydroxytryptamine receptor 3A | TTTGTTGCATGAGTT |
| | (HTR3A), RefSeqGene on chromosome 11 | CGTTCTCTGGGCCTG |
| | | CGGTTCCCCAAGTTC |
| SEQ ID | GI 225007613 NG_011612 Homo sapiens | CAGTCATGCAGAGTA |
| NO: 4042 | upstream transcription factor 1 | GCAATGCTGGGAGTG |
| | (USF1), RefSeqGene on chromosome 1 | CTGGGCCCCTGCTAT |
| | | ATCAATGCTAGGAGG |
| SEQ ID | GI 148227403 NM_005292 Homo sapiens | GAGACAAAGCAGCAA |
| NO: 4043 | G protein-coupled receptor 18 | TTAAAGTCAGCCCAG |
| | (GPR18), transcript variant 1, mRNA | CACCAACTCCGACGC |
| | | CAAGCGTTACACTGG |
| SEQ ID | GI 930697465 NM_005564 Homo sapiens | GGTCCCTCTGCAGCA |
| NO: 4044 | lipocalin 2 | GAACTTCCAGGACAA |
| | (LCN2), mRNA | CCAATTCCAGGGGAA |
| | | GTGGTATGTGGTAGG |
| SEQ ID | GI 542029075 NM_006440 Homo sapiens | ATCACGCCCATTATA |
| NO: 4045 | thioredoxin reductase 2 | AACCACTGGAGTTCA |
| | (TXNRD2), transcript variant 1, mRNA | CGGTGGCTGGACGAG |
| | | ATGCATCCCAGTGTT |
| SEQ ID | GI 31096355 AY300794 Homo sapiens | GGAAGTTCCCCAAAC |
| NO: 4046 | PRKC, apoptosis, WT1, regulator | TCAGAGTAGGACACG |
| | (PAWR) gene, complete cds | TGCTTTTCAGGCTTC |
| | | CTGGCCAGGTCTCAG |
| SEQ ID | GI 306482660 NG_027673 Homo sapiens | AGACACACGAGCTTG |
| NO: 4047 | activating transcription factor 1 | TTTCTGCCTCAGGAT |
| | (ATF1), RefSeqGene on chromosome 12 | CTCTGGAGTTGTCAT |
| | | TGCTCATGCCTGGCA |
| SEQ ID | GI 429535869 NG_033019 Homo sapiens | CCAGTTTGGAGGAAG |
| NO: 4048 | interleukin 5 | GGTCTGAGCACATGT |
| | (IL5), RefSeqGene on chromosome 5 | GGCTGAGCATCCCCA |
| | | TTTCTCTGGAGAAGT |
| SEQ ID | GI 338968872 NG_029246 Homo sapiens | TGTGTGCTCCTTGAG |
| NO: 4049 | RAR related orphan receptor A | GTCAGAGATGTGTCT |
| | (RORA), RefSeqGene on chromosome 15 | TGTGCATCATCAGGG |
| | | ACCCTGGTGCAACAA |
| SEQ ID | GI 386781923 NM_002371 Homo sapiens | TCGGTCTTCACCACC |
| NO: 4050 | mal, T-cell differentiation protein | TTGCCCGACTTGCTC |
| | (MAL), transcript variant a, mRNA | TTCATCTTTGAGTTT |
| | | ATCTTCGGGGGCCTG |
| SEQ ID | GI 384551644 NM_005428 Homo sapiens | GCGCAGGGGAGACTC |
| NO: 4051 | vav guanine nucleotide exchange factor 1 | CTATGACCTCAAGGA |
| | (VAV1), transcript variant 1, mRNA | CTTTGTAAACCTGCA |
| | | CAGCTTCCAGGTTCG |
| SEQ ID | GI 71648775 DQ143945 Homo sapiens | GCGAATCGGGTCTGC |
| NO: 4052 | adrenomedullin | TTGTGTTTTCCAGGT |
| | (ADM) gene, complete cds | GGAATAAGTGGGCTC |
| | | TGAGTCGTGGGAAGA |
| SEQ ID | GI 238018116 NG_012218 Homo sapiens | CAACACTACAGAGCT |
| NO: 4053 | hexose-6-phosphate dehydrogenase/ | GGGGGATCTCAGCCA |
| | glucose 1-dehydrogenase | TTGTTTTCCCAGTGG |
| | (H6PD), RefSeqGene on chro [symbol] | AGACCACTCTTGGCA |
| SEQ ID | GI 296841050 NM_138733 Homo sapiens | AGAACGCTCAGGTTG |
| NO: 4054 | phosphoglycerate kinase 2 | GAAAAGCCACTGTAG |
| | (PGK2), mRNA | CATCTGGCATATCTC |
| | | CTGGCTGGATGGGTT |
| SEQ ID | GI 24756896 L47234 Homo sapiens | CTGAACTGCACAGGT |
| NO: 4055 | ERCC2 | GGCTGTGTGTTTCTG |
| | (ERCC2) and kinesin light chain | GACTCTCCATTCTGT |
| | (KLC2) genes, complete cds, | CCCAGTGGTCTGTCC |
| | complete sequence | |
| SEQ ID | GI 1036030212 AH006319 Homo sapiens | TGTGTCTGGGGCCAG |
| NO: 4056 | succinate dehydrogenase subunit C | CACTGATCCACACAG |
| | (SDHC) gene, complete cds | CTAAGTTTGCACTTG |
| | | TCTTCCCTCTCATGT |
| SEQ ID | GI 237874185 NG_012167 Homo sapiens | GGCATCGGTAAACAG |
| NO: 4057 | CD209 molecule | TCATGGCACTGGAGG |
| | (CD209), RefSeqGene on chromosome 19 | GAGTATCTTATGCCC |
| | | ACGAGCAATGAGGCA |
| SEQ ID | GI 225579058 NM_197965 Homo sapiens | AATAGCAGGGACTAG |
| NO: 4058 | solute carrier family 10 member 6 | CTGGCTGGACTGGCC |
| | (SLC10A6), mRNA | CCCTTCTTTTTCAGT |
| | | GGCCAGTAAAGACAG |
| SEQ ID | GI 227499618 NM_003645 Homo sapiens | AAACACCGCAAAATG |
| NO: 4059 | solute carrier family 27 member 2 | ACCCTGGTGGAGGAG |
| | (SLC27A2), transcript variant 1, mRNA | GGCTTTAACCCTGCT |
| | | GTCATCAAAGATGCC |
| SEQ ID | GI 24371240 NM_024630 Homo sapiens | GCCCGGAGGAAATGG |
| NO: 4060 | zinc finger DHHC-type containing 14 | GAGGTGTTCCCGGGA |
| | (ZDHHC14), transcript variant 1, mRNA | AGAAACAAGTTCTTC |
| | | TGTAACGGGAGGATC |
| SEQ ID | GI 1049010774 AH002843 Homo sapiens | CTGACCACTATGCCC |
| NO: 4061 | interleukin 2 receptor gamma chain | ACTGACTCCCTCAGT |
| | (IL2RG) gene, complete cds | GTTTCCACTCTACCC |
| | | CTCCCAGAGGTTCAG |
| SEQ ID | GI 189217849 NG_007932 Homo sapiens | GGTGTCAGACTGCAG |
| NO: 4062 | cytochrome P450 family 4 | GAAAGGAGCTCACTC |
| | subfamily A member 11 | TGCTGGGGTGGATAT |
| | (CYP4A11), RefSeqGene on chromosome [symbol] | CTGAGGCAGAGATCT |
| SEQ ID | GI 344217739 NG_029805 Homo sapiens | CTCGTTCTGTTCCTG |
| NO: 4063 | DnaJ heat shock protein family | GGGGTGCTTGCTTGT |
| | (Hsp40) member C5 | TCTAGGCCCCCTTTG |
| | (DNAJC5), RefSeqGene on chromosome | TATGGAGAGGTTCAA |
| SEQ ID | GI 161086967 NG_007121 Homo sapiens | CCTCCCCTATTCCAC |
| NO: 4064 | hydroxyacyl-CoA dehydrog | TCCACCTTCACTGGG |
| | enase/ | TTCTCCAGTCAGTAG |
| | 3-ketoacyl-CoA thiolase/ | TTCCCATGTGCCTCT |
| | enoyl-CoA hydratase | |
| | (trifunctiona [symbol] | |
| SEQ ID | GI 164695818 AK315628 Homo sapiens | AGTGGACAGTTGGAG |
| NO: 4065 | cDNA, FU96713, Homo sapiens | GCCTTGAGGTTCTTA |
| | ankyrin repeat and SOCS | GCCAAGTACGGTGCT |
| | box-containing 2 | GACATCAACACGCAG |
| | (ASB2), mRNA | |
| SEQ ID | GI 157427676 NM_004911 Homo sapiens | CACCATCAAGATCCT |
| NO: 4066 | protein disulfide isomerase | TAAGAAGGGGCAGGC |
| | family A member 4 | TGTAGACTACGAGGG |
| | (PDIA4), mRNA | CTCCAGAACCCAGGA |
| SEQ ID | GI 149274622 NM_032579 Homo sapiens | GCCTCCTTCTCATCC |
| NO: 4067 | resistin like beta | TAATCCCCCTTCTCC |
| | (RETNLB), mRNA | AGCTGATCAACCCGG |
| | | GGAGTACTCAGTGTT |
| SEQ ID | GI 163644319 NM_032873 Homo sapiens | CGGAGGTCCTCAAGA |
| NO: 4068 | ubiquitin associated and | AGTTTGCTGCTGACT |
| | SH3 domain containing B | TTGCTGCAGAGGCTG |
| | (UBASH3B), mRNA | CATCCAAAACCGAAG |
| SEQ ID | GI 52854080 AY764389 Homo sapiens | GGGCCTTTGCAGGTG |
| NO: 4069 | baculoviral lAP repeat-containing 3 | CCAAATAAATTTCAG |
| | (BIRC3) gene, complete cds | GACAGAGCCTAAGGC |
| | | AGAGCTCTGGCACAG |
| SEQ ID | GI 4572463 AF123653 Homo sapiens | CCCCTGAAGCAGAAG |
| NO: 4070 | FEZI | TCTTGGGTCAGGCCA |
| | (FEZl) gene, complete cds | TAAGCAAAGAGCACA |
| | | GGGGATATGTGAGCT |
| SEQ ID | GI 297747369 NG_021416 Homo sapiens | GGAAGGATGAGGACT |
| NO: 4071 | growth hormone releasing | CCAAACAGCCAGCTC |
| | hormone receptor | CCATGCCAAATAGAA |
| | (GHRHR), RefSeqGene on chromosome 7 | CGATGAGTGCTGGGA |
| SEQ ID | GI 344925847 NG_029819 Homo sapiens | GCAGATTGACAGCCC |
| NO: 4072 | prolactin | ACAGGTGTACGCGAT |
| | (PRL), RefSeqGene on chromosome 6 | GTGACAAGACGACGT |
| | | GCAAAGACCACAGTA |
| SEQ ID | GI 315434201 NM_020429 Homo sapiens | TGCAGCCAGGTTTAG |
| NO: 4073 | SMAD specific E3 | CCTTTGGCTTTGGTC |
| | ubiquitin protein ligase 1 | ACACAGGATATTCTG |
| | (SMURF1), transcript variant 1, mRNA | CTGTGTTGCAACCCA |
| SEQ ID | GI 1028630732 NG_050570 Homo sapiens | TCCGCCTGACTGAGC |
| NO: 4074 | carnitine palmitoyltransferase 1C | TGGGAACCATTGCTT |
| | (CPT1C), RefSeqGene on chromosome 19 | CCTTGCTGTGTGACC |
| | | TTCTGCACGTTATTA |
| SEQ ID | GI 10798850 AF084559 Homo sapiens | ATGGCAGCCCATCGA |
| NO: 4075 | fatty acid desaturase 2 | GTACGGCAAGAAGAA |
| | (FADS2) mRNA, complete cds | GCTGAAATACCTGCC |
| | | CTACAATCACCAGCA |
| SEQ ID | GI 156104861 NM_181712 Homo sapiens | GGCTACACTGCCGTA |
| NO: 4076 | KN motif and ankyrin repeat domains 4 | ATGATCACTCCCTTG |
| | (KANK4), transcript variant 1, mRNA | GCTTCCGCAGAGACC |
| | | AATGAAGACATGGCT |
| SEQ ID | GI 214010161 NG_009030 Homo sapiens | TGAGATCACACATTG |
| NO: 4077 | GLI family zinc finger 2 | AGTTGATGGCAAAAG |
| | (GLI2), RefSeqGene on chromosome 2 | CAGAAGCCTAGGCCT |
| | | CCCGCCTGCAAATGC |
| SEQ ID | GI 237858740 NG_012111 Homo sapiens | CCTCATCTGTAAAGT |
| NO: 4078 | platelet derived growth | GTGGGTAACAGCATC |
| | factor subunit B | TGCCGGTAACAGCAC |
| | (PDGFB), RefSeqGene on chromosome 22 | CTGCCGTGAAGGGAA |
| SEQ ID | GI 195927007 NM_052813 Homo sapiens | ACTACGTAAGGACTC |
| NO: 4079 | caspase recruitmen | CAAGATGTACAAGGA |
| | t domain family member 9 | CCGCATCGAGGCCAT |
| | (CARD9), transcript variant 1, mRNA | CCTGCTGCAGATGGA |
| SEQ ID | GI 815890844 NM_001836 Homo sapiens | GACTCATGGATCCCC |
| NO: 4080 | chymase 1 | AGGCCTGCAGCCACT |
| | (CMAl), transcript variant 1, mRNA | TCAGAGACTTTGACC |
| | | ACAATCTTCAGCTGT |
| SEQ ID | GI 194018737 NG_008190 Homo sapiens | CAGACTTCCAGCATG |
| NO: 4081 | acyl-CoA oxidase 1 | AGGACTTGAACTTCC |
| | (ACOX1), RefSeqGene on chromosome 17 | TCACTCGCAGCCAGC |
| | | GTTATGAGGTGGCTG |
| SEQ ID | GI 533112518 NG_007159 Homo sapiens | TGGGCAGGGGACAGG |
| NO: 4082 | chloride voltage-gated channel 5 | CCACACAGGGTTTTA |
| | (CLCN5), RefSeqGene on chromosome X | TAGGCCATGTTACCA |
| | | AGTACCCTAACTTGG |
| SEQ ID | GI 284813593 NG_016440 Homo sapiens | CTGCCAGCACTTTCT |
| NO: 4083 | MAF bZIP transcription factor | TCGGCCCCTTTCCTA |
| | (MAF), RefSeqGene on chromosome 16 | TAGGGTCGGGTTCTC |
| | | CCACTTCAGTCAAAC |
| SEQ ID | GI 22538815 NM_005623 Homo sapiens | GAGGTCACTTTGCTG |
| NO: 4084 | C-C motif chemokine ligand 8 | AGCACAATCCCATAC |
| | (CCL8), mRNA | CCTTCAGCCTCTGCT |
| | | CCACAGAGCCTAAGC |
| SEQ ID | GI 593023777 KJ145927 Homo sapiens | TTACTCGCTGGCCTG |
| NO: 4085 | lamin A/C | GCCTTTCTTCTCTCT |
| | (LMNA) gene, complete cds | CCTCCCTATACCTTG |
| | | AACAGGGAACCCAGG |
| SEQ ID | GI 575006674 NG_009015 Homo sapiens | CTTCCTGTGCCCTGA |
| NO: 4086 | glucose-6-phosphate dehydrogenase | ATGGGGAGTGGCACC |
| | (G6PD), RefSeqGene on chromosome X | TCTTGAGTTTTCAGA |
| | | GGGAGGAAGTATGCC |
| SEQ ID | GI 2789671 AF040714 Homo sapiens | TATGGAGGAACTGCT |
| NO: 4087 | homeobox protein A10 | TGCTCTCAGCAAGCC |
| | (HOXA10) gene, complete cds | AAAAACGGGGGCACG |
| | | ACTCTCTTCTCTGTG |
| SEQ ID | GI 224451013 NG_009888 Homo sapiens | ATCCCATCAGCTCTG |
| NO: 4088 | acetyl-CoA acetyltransferase 1 | GCCAGAGGCAGGGGA |
| | (ACAT1), RefSeqGene on chromosome 11 | GTCACCTTGTAAGTA |
| | | CTTAGGGTTGTCCTT |
| SEQ ID | GI 227908850 NG_011958 Homo sapiens | TTTCGGAGGGGTCCG |
| NO: 4089 | noggin | AATCTGGGTGAAAAT |
| | (NOG), RefSeqGene on chromosome 17 | CACCACCTCTGGCAT |
| | | CTGCTTTTGGGAAGA |
| SEQ ID | GI 346227162 NG_029971 Homo sapiens | CAGACACACCCTCAG |
| NO: 4090 | RELA proto-oncogene, NF-kB subunit | TGACCACAGCCTTCC |
| | (RELA), RefSeqGene on chromosome 11 | ACACTAGTCCACAAC |
| | | CAGGAAAAAGTGGCA |
| SEQ ID | GI 197304706 NM_032350 Homo sapiens | CTCCTGCTGCACATG |
| NO: 4091 | chromosome 7 open reading frame 50 | TATGACAGTGACAAG |
| | (C7orf50), transcript variant 1, mRNA | GTTCCCGATGAGCAC |
| | | TTCTCCACCCTGCTG |
| SEQ ID | GI 242247668 NM_032962 Homo sapiens | TCTTCCTCCTCATCA |
| NO: 4092 | C-C motif chemokine ligand 14 | CCATCGCCCTAGGGA |
| | (CCL14), transcript variant 2, mRNA | CCAAGACTGAATCCT |
| | | CCTCACAAACTGGGG |
| SEQ ID | GI 242247668 NM_032962 Homo sapiens | CCGCGTCAGCGGATT |
| NO: 4093 | C-C motif chemokine ligand 14 | ATGGATTACTATGAG |
| | (CCL14), transcript variant 2, mRNA | ACCAACAGCCAGTGC |
| | | TCCAAGCCCGGAATT |
| SEQ ID | GI 658309997 NM_000853 Homo sapiens | TTGCACCGTCCTCAG |
| NO: 4094 | glutathione S-transferase theta l | CAGTCCACAAAGCAT |
| | (GSTT1), transcript variant 1, mRNA | TTTCATTTCTAATGG |
| | | CCCATGGGAGCCAGG |
| SEQ ID | GI 270309111 NG_016167 Homo sapiens | ACAGGCAGGGAGATG |
| NO: 4095 | glycogen synthase 2 | CAGGTCATGCAGGTC |
| | (GYS2), RefSeqGene on chromosome 12 | AATATACCCATGAGG |
| | | AGAGAGCAGGAAGGA |
| SEQ ID | GI 209870107 NG_008768 Homo sapiens | GGAGGGAAAGCAACC |
| NO: 4096 | propionyl-CoA carboxylase alpha subunit | TGAAGGATCCATCTG |
| | (PCCA), RefSeqGene on chromosome 13 | CTCCCTTCTGCTACA |
| | | CTGCCTCGATGCAAG |
| SEQ ID | GI 1018191682 NG_012337 Homo sapiens | GGACCCATTCCTTGC |
| NO: 4097 | succinate dehydrogenase complex subunit D | CAGTTTGGGGGGTCT |
| | (SDHD), RefSeqGene on chromosome 11 | TTAGCGGTTGGGAAG |
| | | TAGCTCTCAAACTCT |
| SEQ ID | ref\|NG_021140.1\| Homo sapiens | ATGCCCAAAGAGGAG |
| NO: 4098 | adiponectin, CIQand collagen domain containing | AGAGGAAGCTCTCTT |
| | | TCCCAGATGCCCCAG |
| | | CAAGTGTAACCTTGC |
| SEQ ID | GI 190358530 NG 008050 Homo sapiens | TACCAGGTAGCTGGG |
| NO: 4099 | endothelin 3 | GCCTGTAGGAGTCAG |
| | (EDN3), RefSeqGene on chromosome 20 | GGTTCTCTAGAGAGA |
| | | CAGAACCAACAGGGT |
| SEQ ID | GI 341823675 NG_029547 Homo sapiens | CCCTCACATCACTCA |
| NO: 4100 | interleukin 5 receptor subunit alpha | GAGAGACTTACAGGA |
| | (IL5RA), RefSeqGene on chromosome 3 [symbol] | GCAGCATGAGCCTTC |
| | | CTGGGGAGAGGGAAC |
| SEQ ID | GI 224809246 NG_011451 Homo sapiens | GAAGGCAGGCACGGT |
| NO: 4101 | integrin subunit alpha X | CTCACAGCTAACATT |
| | (ITGAX), RefSeqGene on chromosome 16 | TACAGAGCAGTAAGT |
| | | GCAGTGCCAGGCTCA |
| SEQ ID | GI 224809344 NG_011510 Homo sapiens | GTCCTGCTATTGGTC |
| NO: 4102 | lanosterol synthase | TAGGGGATCAGCTAG |
| | (LSS), RefSeqGene on chromosome 21 [symbol] | CCTTGCCAGTGTAGG |
| | | GTGACAGGCTCTCTG |
| SEQ ID | GI 181979 M29366 Human | TGGCTCGGGTGTCTT |
| NO: 4103 | epidermal growth factor receptor | TGGAACTGTGCACAA |
| | (ERBB3) mRNA, complete cds [symbol] | AGGAGTGTGGATCCC |
| | | TGAGGGTGAATCAAT |
| SEQ ID | GI 189458808 NG_007982 Homo sapiens | CCACAGCAGGGATGG |
| NO: 4104 | cytochrome P450 family 19 | GAACTATAGGGTGGA |
| | subfamily A member 1 | CAAGGCATTTGCCCT |
| | (CYP19A1), RefSeqGene on chromosome [symbol] | GAAGGTAGTCCACAC |
| SEQ ID | GI 345478701 NG_029901 Homo sapiens | TTGTCACCAGCCAGC |
| NO: 4105 | peroxiredoxin 2 | CTGGGTTCAAATGCC |
| | (PRDX2), RefSeqGene on chromosome 19 [symbol] | GTTTAAGAGCTGTGA |
| | | TCGTCTCTGGGTCTC |
| SEQ ID | GI 62530382 NM_004281 Homo sapiens | GTAAGCCAGGCCCAG |
| NO: 4106 | BCL2 associated athanogene 3 | TTGGACCAGAACTCC |
| | (BAG3), mRNA [symbol] | CTCCTGGACACATCC |
| | | CAATTCAAGTGATCC |
| SEQ ID | GI 157427676 NM_004911 Homo sapiens | TTACAGAGCTGCAAC |
| NO: 4107 | protein disulfide isomerase | TCAGTTTTGGCGGAG |
| | family A member 4 | CAAAGTCCTAGAGGT |
| | (PDIA4), mRNA [symbol] | GGCCAAGGACTTCCC |
| SEQ ID | GI 544583587 NM_004683 Homo sapiens | CTGGTCTGGGGGTCA |
| NO: 4108 | regucalcin | AAGGAATTGCTCCCT |
| | (RGN), transcript variant 1, mRNA [symbol] | ACTCCTATGCGGGAT |
| | | GAGGACAGGTCTTCT |
| SEQ ID | GI 37499464 AY422952 Homo sapiens | TGTGGCTTTGCCTGG |
| NO: 4109 | apolipoprotein A-1 | TCCGCTGTGACTTCC |
| | (APOA1) gene, complete cds [symbol] | TTTCTACTTGAGCCT |
| | | TGCTAAGGCAGACTC |
| SEQ ID | GI 117606579 EF064764 Homo sapiens | AGTGAAGGAATCCTG |
| NO: 4110 | chemokine | GGTAGAGACCATTCT |
| | (C-C motif) ligand 22 | CAGGTGGTTGGGCCA |
| | (CCL22) gene, complete cds [symbol] | GGCTAAAGACTGGGA |
| SEQ ID | GI 50234968 AY665769 Homo sapiens | TGGTCAGAACCTAGG |
| NO: 4111 | chemokine-like factor | AGGCAGTGGCTTGAG |
| | (CKLF) gene, complete cds [symbol] | ACTCTGGGTGGAAAA |
| | | GGAGACTGTGAGAGC |
| SEQ ID | GI 13430063 AF253322 Homo sapiens | CAGAGCTCTCTGAGT |
| NO: 4112 | cytochrome P450 | GACTAAAGCCTGGTA |
| | (CYP1A1) and cytochrome P450 | CTTGCAGGCCCCAAG |
| | (CYP1A2) genes, complete cds [symbol] | AAGACCTACCCAGGT |
| SEQ ID | GI 57864635 AY882422 Homo sapiens | CCATAGCTTTAGGGC |
| NO: 4113 | flavin containing monooxygenase 4 | TTTGCCAGAACTATG |
| | (FMO4) gene, complete cds | ACGTTGAGGAGGGTG |
| | | GGGTAAAGGGGCAGG |
| SEQ ID | GI 82583657 DQ297142 Homo sapiens | GCAGGTGGAGGCACA |
| NO: 4114 | peroxiredoxin 1 | GTGATGGGAAAGGTC |
| | (PRDX1) gene, complete cds | AACGTGACTTTTGGG |
| | | GAGCCAAACAAAAGT |
| SEQ ID | GI 237820648 NG_012041 Homo sapiens | CCTGTGCCAGGTTTT |
| NO: 4115 | adenosine monophosphate deaminase 3 | GAGGTTGGGCAAGGG |
| | (AMPD3), RefSeqGene on chromosome 11 | AAAGAGCATTCAGTT |
| | | GTGCTTGGAGGAACA |
| SEQ ID | GI 588282795 NM_002168 Homo sapiens | TTTTGCGCACAGCTG |
| NO: 4116 | isocitrate dehydrogenase | CTTCCAGTATGCCAT |
| | (NADP(+)) 2, mitochondrial | CCAGAAGAAATGGCC |
| | (IDH2), transcript variant 1, mRNA | GCTGTACATGAGCAC |
| SEQ ID | GI 117606358 NM_206909 Homo sapiens | TGCGATGGGCTCTTC |
| NO: 4117 | pleckstrin and Sec7 domain containing 3 | TTGGTGTCTGTATGG |
| | (PSD3), transcript variant 2, mRNA | TTGCTGCAATGCTGG |
| | | GGTGAAAACAACACG |
| SEQ ID | GI 85725379 DQ370420 Homo sapiens | GATGTTCAGTCAAAG |
| NO: 4118 | metallothionein 2A | AGGAGGATGAGTGCC |
| | (MT2A) gene, complete cds | TTGGCCCAGGAATCA |
| | | GGAGCACACTTGCCT |
| SEQ ID | GI 269308199 NG_015892 Homo sapiens | CTGGTGCCTTTGCTC |
| NO: 4119 | bassoon presynaptic cytomatrix protein | CAGCCACTTGGAAGA |
| | (BSN), RefSeqGene on chromosome 3 [symbol] | GATTAGAGTCCCCAA |
| | | ACCCCACAGAGTCAC |
| SEQ ID | GI 209529670 NG_008721 Homo sapiens | AGTCCCTTCCCCAAA |
| NO: 4120 | LIF, interleukin 6 family cytokine | GTCACTCAGCTCATA |
| | (LIF), RefSeqGene on chromosome 22 [symbol] | ACTGGCAGAGCCAAA |
| | | CCTCCAACCTGGTTG |
| SEQ ID | GI 336455046 NG_029166 Homo sapiens | TTGGGGTCTGCTCTA |
| NO: 4121 | SRY-box 4 | AGCTGCAGCAAGAGA |
| | (SOX4), RefSeqGene on chromosome 6 [symbol] | AACTGTGTGTGAGGG |
| | | GAAGAGGCCTGTTTC |
| SEQ ID | GI 4826562 Y17151 Homo sapiens | ACATGCTGCCCCAGT |
| NO: 4122 | mRNA for multidrug resistance protein 3 | TAATCAGCAACCTGA |
| | (ABCC3) [symbol] | CTCAGGCCAGTGTGT |
| | | CTCTGAAACGGATCC |
| SEQ ID | GI 658309997 NM_000853 Homo sapiens | CAGAGTTGGATGTGA |
| NO: 4123 | glutathione S-transferase theta l | CCCTGCAGTTGCTCG |
| | (GSTT1), transcript variant 1, mRNA [symbol] | AGGACAAGTTCCTCC |
| | | AGAACAAGGCCTTCC |
| SEQ ID | GI 82734235 NM_006084 Homo sapiens | GGATCAGAGGTCCCT |
| NO: 4124 | interferon regulatory factor 9 | GGAGTTTCTGCTTCC |
| | (IRF9), mRNA [symbol] | TCCAGAGCCAGACTA |
| | | CTCACTGCTGCTCAC |
| SEQ ID | GI 393662586 NM_002754 Homo sapiens | TGGCCTGGCATTCAA |
| NO: 4125 | mitogen-activated protein kinase 13 | AACCCCTTGTGTTCT |
| | (MAPK13), transcript variant 1, mRNA [symbol] | CTCTAATCTCATCAG |
| | | CTCCCACTGCCTCCC |
| SEQ ID | GI 117606585 EF064767 Homo sapiens | TGAATGAAAGGAGAA |
| NO: 4126 | chemokine | ATGGGAGGGCAGCAT |
| | (C-C motif) ligand 17 | GCGAGGGTGTTGAAG |
| | (CCL17) gene, complete cds [symbol] | TCAAGGGGAGGTCTT |
| SEQ ID | GI 57790271 AY878121 Homo sapiens | GGTGAGAGCTTTGAG |
| NO: 4127 | glutathione S-transferase A4 | CCACACCAGCAATGT |
| | (GSTA4) gene, complete cds [symbol] | GATACTGGGTGAGAG |
| | | CTCTGGTTCACGTGG |
| SEQ ID | GI 20303097 AF503166 Homo sapiens | GTCCCTGCTGCAGAC |
| NO: 4128 | xeroderma pigmentosum, complementation group A | ATTGGGAGCCATCAT |
| | (XPA) gene, complete cds [symbol] | GATTGGCTTGTTGCT |
| | | GAGCAGAATCCTTGG |
| SEQ ID | GI 564730481 NM_001287262 Homo sapiens | GAGTCACAGGGTCAC |
| NO: 4129 | butyrophilin like 10 | CACCTTTGACAACAG |
| | (BTNL10), mRNA [symbol] | GACATACTGCTGCCG |
| | | CTTCAAGGATGGTGT |
| SEQ ID | GI 27477078 NM_013278 Homo sapiens | GGGTAGAAGGAGCTC |
| NO: 4130 | interleukin 17C | AGCACCTCTTCCAGC |
| | (IL17C), mRNA [symbol] | CCTTAAAGCTGCAGA |
| | | AAAGGTGTCACACGG |
| SEQ ID | GI 10280531 AB038161 Homo sapiens | GATTTCCAAGAGCAG |
| NO: 4131 | ABCG1 gene for ABC transporter | CTTGCAGCCTTGCTC |
| | (ATP-binding cassette, sub-family G | ACAGGAAGGCACCAC |
| | (WHITE), member 1), com [symbol] | GTTGATGCAGAGTTT |
| SEQ ID | GI 699969198 NG_041780 Homo sapiens | CAATTCCTCCGGTAC |
| NO: 4132 | NLR family CARD domain containing 4 | ACCAAGGCAAGAACC |
| | (NLRC4), RefSeqGene on chromosome 2 [symbol] | CCGGAATACAGAAAG |
| | | CCCTCTGTCCTTGCA |
| SEQ ID | GI 654825134 NG_008781 Homo sapiens | GCCACTCCACGACCT |
| NO: 4133 | poliovirus receptor | TACTTTGCTGGCATG |
| | (PVR), RefSeqGene on chromosome 19 [symbol] | AGTGCTGGACAGCAA |
| | | TTTGGCAACATCCTC |
| SEQ ID | GI 2660728 AFO31523 Homo sapiens | ATGTTCCAGATCCCA |
| NO: 4134 | bcl-xL/bcl-2 associated death promoter | GAGTTTGAGCCGAGT |
| | (BAD) mRNA, complete cds [symbol] | GAGCAGGAAGACTCC |
| | | AGCTCTGCAGAGAGG |
| SEQ ID | GI 32479526 NM_000218 Homo sapiens | CACCCTGTACATCGG |
| NO: 4135 | potassium voltage-gated | CTTCCTGGGCCTCAT |
| | channel subfamily Q member 1 | CTTCTCCTCGTACTT |
| | (KCNQ1), transcript variant 1, | TGTGTACCTGGCTGA |
| | mRNA [symbol] | |
| SEQ ID | GI 1006833939 NM_003978 Homo sapiens | GCACAGCAACCAGCT |
| NO: 4136 | proline-serine-threonine | CTCCATGCAGTGTGT |
| | phosphatase interacting protein 1 | CAAGGATGATGAGCT |
| | (PSTPIP1), transcript variant 1, [symbol] | CTACGAGGAAGTGCG |
| SEQ ID | GI 269954706 NG_015999 Homo sapiens | GCCCAAACCCTGCTG |
| NO: 4137 | enoyl-CoA hydratase and | CCATCCTGGTTGTCC |
| | 3-hydroxyacyl CoA dehydrogenase | TCATCGTCTCTATGG |
| | (EHHADH), RefSeqGene on chro [symbol] | ACAATCATTCCCTCC |
| SEQ ID | GI 342307072 NG_029607Homo sapiens | CTTATGACTCAAAGG |
| NO: 4138 | heat shock transcription factor 2 | CCCTGAAGGGTTTCT |
| | (HSF2), RefSeqGene on chromosome 6 [symbol] | GTAGCCTCCCAGCCA |
| | | CAGCCCAGAAACCAT |
| SEQ ID | GI 748585188 NM_004040 Homo sapiens | ATGTTCGCCCTTCAC |
| NO: 4139 | ras homolog family member B | CAGCGGGAGCTTGAT |
| | (RHOB), mRNA [symbol] | ATCCCTTGTCTGTAA |
| | | CATAGACCCCGGGTA |
| SEQ ID | GI 340805891 NG_029487Homo sapiens | TGGGGTAGGAAGTCT |
| NO: 4140 | BCL2 related protein A1 | TCCATGACTGTGCAG |
| | (BCL2A1), RefSeqGene on chromosome 15 [symbol] | TCGAAGGAAGTTTCA |
| | | GCAAAGCCCATGGGG |
| SEQ ID | GI 223972691 NG 009823Homo sapiens | TCCACACACACCACA |
| NO: 4141 | crystallin alpha A | GACACACACACACCC |
| | (CRYAA), RefSeqGene on chromosome 21 [symbol] | CTCAACACACCATGC |
| | | CCTTTGTCTCTCTTT |
| SEQ ID | GI 1016841222 NG_008723 Homo sapiens | AGTTGGGGTCCATGC |
| NO: 4142 | monoamine oxidase B | CTCCTCTCTATGAAT |
| | (MAOB), RefSeqGene on chromosome X | CTGGGAGGACTCTGT |
| | | CACGGCTTTGACCAG |
| SEQ ID | GI 237858716 NG_012099 Homo sapiens | AGCAATGGGGCTGGG |
| NO: 4143 | macrophage migration inhibitory factor | TGGGGTGAAGATTAG |
| | (MIF), RefSeqGene on chromosome 22 [symbol] | GAAGAGGAAGGAGAA |
| | | TAGAAGCTCCAGGGA |
| SEQ ID | GI 339639630 NG_029383 Homo sapiens | AGCCATAGCACCAAG |
| NO: 4144 | mucin 1, cell surface associated | ACTGATGCCAGTAGC |
| | (MUC1), RefSeqGene on chromosome 1 [symbol] | ACTCACCATAGCACG |
| | | GTACCTCCTCTCACC |
| SEQ ID | GI 237858596 NG_012143 Homo sapiens | CCAGGTAGGGACAGA |
| NO: 4145 | nuclear receptor subfamily 0 group B member 2 | CCAGGATGGCAACTC |
| | (NROB2), RefSeqGene on chromosome 1 [symbol] | AGGTCTATTCACTCC |
| | | AGAGACTGGCCCTTG |
| SEQ ID | GI 60391308 D50017 Homo sapiens | AGTGGTAGGTACCCT |
| NO: 4146 | PDGFRA gene for alpha-platelet-derived | CAAAACGTGCAATGG |
| | growth factor receptor, complete cds [symbol] | CTTGGAGCAGAGCAA |
| | | CAGGGCTCAGAAGAC |
| SEQ ID | GI 269954724 NG_016003 Homo sapiens | ACAGTGGTATACAGT |
| NO: 4147 | interferon alpha and beta receptor subunit 2 | GGCATTAAGGGGTGA |
| | (IFNAR2), RefSeqGene on chromosome 21 [symbol] | AAAGGTGGAAGCAGC |
| | | GAGGTTGGGTGCAGT |
| SEQ ID | GI 260593670 NG_013025 Homo sapiens | CAGATCCAGGGACTG |
| NO: 4148 | nucleotide binding oligomerization | GGCACAAACATCTGC |
| | domain containing 1 | CTGTTGAGTAGGAAC |
| | (NOD1), RefSeqGene on chromoso [symbol] | TGAGCTGTGGCCATT |
| SEQ ID | GI 194239686 NM_004591 Homo sapiens | TGCTGTACCAAGAGT |
| NO: 4149 | C-C motif chemokine ligand 20 | TTGCTCCTGGCTGCT |
| | (CCL20), transcript variant 1, mRNA [symbol] | TTGATGTCAGTGCTG |
| | | CTACTCCACCTCTGC |
| SEQ ID | GI 313569850 NM_144601 Homo sapiens | TCCTTCTCCCCATTT |
| NO: 4150 | CKLF like MARVEL transmembrane | CTGTCCCACAGGCCT |
| | domain containing 3 | TCAGCCCTTTAACGT |
| | (CMTM3), transcript variant 1, mRN [symbol] | CTCTGCCAAAAACCA |
| SEQ ID | GI 874507471 NM_005928 Homo sapiens | AAACCCCTGCCACAA |
| NO: 4151 | milkfat globule-EGF factor 8 protein | CGGTGGTTTATGCGA |
| | (MFGE8), transcript variant 1, mRNA [symbol] | GGAGATTTCCCAAGA |
| | | AGTGCGAGGAGATGT |
| SEQ ID | GI 325652102 NM_002811 Homo sapiens | TCAACCGAATCGGCA |
| NO: 4152 | proteasome 26S subunit, non-ATPase 7 | AGGTTGGAAACCAGA |
| | (PSMD7), mRNA [symbol] | AGCGTGTTGTTGGTG |
| | | TGCTTTTGGGGTCAT |
| SEQ ID | GI 1060099004 NM_001330400 Homo sapiens | GAAGCAGCATCAACC |
| NO: 4153 | sideroflexin 5 | TAGGAGCAGTGGGCC |
| | (SFXN5), transcript variant 1, mRNA | CTGGCTCTGTCACTA |
| | | AATGGCAGTGAGATG |
| SEQ ID | GI 197245396 NG_008399 Homo sapiens | AACAGCCCACATTGG |
| NO: 4154 | aspartoacylase | CCTGCTGGATAATGA |
| | (ASPA), RefSeqGene on chromosome 17 | AAGCCTTTGAGGTGG |
| | | AAAACTCAGGCACCC |
| SEQ ID | GI 336455032 NG_029156 Homo sapiens | GGCTATCCCAGTGGC |
| NO: 4155 | betaine-homocysteine S-methyltransferase | TTGTTGGGAGGGGTA |
| | (BHMT), RefSeqGene on chromosome 5 | TTTTCAGTCCAGACG |
| | | GTAGGCCTTCCTCTA |
| SEQ ID | GI 269954730 NG_016009 Homo sapiens | GGAGTCAGGAGACTC |
| NO: 4156 | kininogen 1 | AGTTCCACCATAGCA |
| | (KNG1), RefSeqGene on chromosome 3 | TCTGCCGCTCACATC |
| | | CCAGATCACTCTGGG |
| SEQ ID | GI 347658934 NG_030028 Homo sapiens | AAATGTGGGCTTCCT |
| NO: 4157 | mitochondrial antiviral signaling protein | TTCCTTTTGTTTGGG |
| | (MAVS), RefSeqGene on chromosome 20 | CCACATCATCCCTTC |
| | | CCCTCCATCTGTGGC |
| SEQ ID | GI 584458494 NG_033933 Homo sapiens | AAGTCGTTTTTGCCA |
| NO: 4158 | toll like receptor 9 | CACTGTGGGGTGTTG |
| | (TLR9), RefSeqGene on chromosome 3 | GGGTCACGTGTTTGC |
| | | AGGTTTGGGGAATTA |
| SEQ ID | GI 554506569 NM_001114 Homo sapiens | GTCGCTGCATGCCTG |
| NO: 4159 | adenylate cyclase 7 | ACAACAACTTCCACA |
| | (ADCY7), transcript variant 1, mRNA | GCCTCTACGTCAAGA |
| | | GGCACCAGAATGTCA |
| SEQ ID | GI 54112075 AY771350 Homo sapiens | CCACAGAAGCAAAGC |
| NO: 4160 | heme oxygenase | ATTGCTCATTTGGGT |
| | (decycling) 2 | GGTTCCCAACACAGA |
| | (HMOX2) gene, complete cds | CACATGGACGTGGGT |
| SEQ ID | GI 209447054 NG_008716 Homo sapiens | ACATAACCTGCTGGA |
| NO: 4161 | aminolevulinate dehydratase | AGGCAGGGACTCCAC |
| | (ALAD), RefSeqGene on chromosome 9 | TTCAGAAATCTTGGA |
| | | AAACCCTGTGGCCCA |
| SEQ ID | GI 294610641 NG_017089 Homo sapiens | AGCTGGGATTCTTCT |
| NO: 4162 | galectin 3 | TCAGAGCTTCAAGCG |
| | (LGALS3), RefSeqGene on chromosome 14 | GGGCCAGGCCTTTTT |
| | | ATACCCCCACTTTGA |
| SEQ ID | GI 269914179 NG_015979 Homo sapiens | TCTCAGTAGGCACAA |
| NO: 4163 | nitric oxide synthase 1 adaptor protein | GGCCAAAGTCAGCCG |
| | (NOSIAP), RefSeqGene on chromosome 1 | ACTTGCAGATTGTAA |
| | | TGCCGGTTCTCCCAC |
| SEQ ID | GI 195234781 NG_008319 Homo sapiens | TGGGTCTCTGGGCTG |
| NO: 4164 | pyruvate carboxylase | TCAGCTCTCTGGAGA |
| | (PC), RefSeqGene on chromosome 11 | GGAAAACAGGGTGAA |
| | | GGATCTTTGGGTGAT |
| SEQ ID | GI 342307060 NG_029601 Homo sapiens | GACTCGGGCTTGTGG |
| NO: 4165 | Pim-1 proto-oncogene, | TTTATGTTTATTTGC |
| | serine/threonine kinase | CCTTGGAGGACGCTG |
| | (PIM1), RefSeqGene on chromosome 6 | GGTTGGGCTGCATTT |
| SEQ ID | GI 62953114 NM_012342 Homo sapiens | GCGGCAACAGATGCT |
| NO: 4166 | BMP and activin membrane bound inhibitor | CTCCCGTTTGCACTA |
| | (BAMBI), mRNA | CAGCTTTCACGGACA |
| | | CCATTCCAAAAAGGG |
| SEQ ID | GI 145312264 NM_033266 Homo sapiens | CAAGCAGACAGGGGA |
| NO: 4167 | endoplasmic reticulum | CCTGAAGTGGACTCT |
| | to nucleus signaling 2 | GAGGGATGATCCCGT |
| | (ERN2), transcript variant 1, mRNA | CATCGAAGGACCAAT |
| SEQ ID | GI 27979118 AY219866 Homo sapiens | ACGCGTCACTCTTCT |
| NO: 4168 | caspase 3, apoptosis- | GCCCAACTTCATAGC |
| | related cysteine protease | CTCCTCTTGCTGGCC |
| | (CASP3) gene, complete cds | TCCTATCTGTATGCT |
| SEQ ID | GI 476704 L26336 Homo sapiens | TGAACCCCACCAACA |
| NO: 4169 | heat shock protein | CCATCTTCGACGCCA |
| | (HSPA2) gene, complete cds | AGAGGCTGATTGGAC |
| | | GGAAATTCGAGGATG |
| SEQ ID | GI 66841734 DQ073384 Homo sapiens | AGACATTTTGGTGGT |
| NO: 4170 | phosphatase and tensin homolog | CACAGCTGGGGTGGT |
| | (mutated in multiple advanced cancers 1) | AGAGTGTGCTATTGG |
| | (PTEN) gene, comp [symbol] | CTAGGGGCTTGAAGC |
| SEQ ID | GI 1150763505 NG_052952 Homo sapiens | GGGGCCTTCATTAGG |
| NO: 4171 | ezrin | GCGGTTAGTTTGAAG |
| | (EZR), RefSeqGene on chromosome 6 | GCTGTCCTGGAAGTT |
| | | GGAGAGTGACATGGG |
| SEQ ID | GI 299782605 NG_023187 Homo sapiens | ATGAGCACAAGGACT |
| NO: 4172 | major histocompatibility | GAACCCTGGACCAGT |
| | complex, class 1, B | TCTAAGGGGTGTGAT |
| | (HLA-B), RefSeqGene on chromosome 6 | CAGACCACACCCAGA |
| SEQ ID | GI 1114655579 NM_002969 Homo sapiens | AACGTGATCGGGCTG |
| NO: 4173 | mitogen-activated protein kinase 12 | CTGGACGTATTCACT |
| | (MAPK12), transcript variant 1, mRNA | CCTGATGAGACCCTG |
| | | GATGACTTCACGGAC |
| SEQ ID | GI 208431777 NM_001135638 Homo sapiens | TCATGCAGGGAAGTT |
| NO: 4174 | phosphatidylinositol-4- | GGAAATGGGGGCTAC |
| | phosphate 5-kinasetype 1 alpha | ATATGCCCTCTCCTC |
| | (PIP5K1A), transcript variant 1, [symbol] | CCCGTCTACAAGAGT |
| SEQ ID | GI 281485607 NM_003226 Homo sapiens | CCCAAGCAAACAATC |
| NO: 4175 | trefoil factor 3 | CAGAGCAGCTGTGCA |
| | (TFF3), mRNA | AACAACGGTGCATAA |
| | | ATGAGGCCTCCTGGA |
| SEQ ID | GI 85725379 DQ370420 Homo sapiens | AGCCAGGCTACTGCA |
| NO: 4176 | metallothionein 2A | CTGAGCTTCAGCTTC |
| | (MT2A) gene, complete cds | TGTAAAACTGAATGG |
| | | CGCTGTGCCAGATCA |
| SEQ ID | GI 343098522 NG_029664 Homo sapiens | CTGGCCTGCGTTCAC |
| NO: 4177 | cathepsin E | ACTGTTTCAGCATAA |
| | (CTSE), RefSeqGene on chromosome 1 | GCCCATTCCTCTCAT |
| | | TCCCCTCTCCCACAT |
| SEQ ID | GI 761631399 NG_042041 Homo sapiens | AGCACACTCCCATCA |
| NO: 4178 | integrin subunit beta 6 | CTACCTGCCTGCCTA |
| | (ITGB6), RefSeqGene on chromosome 2 | TGGAATGCTGACAAT |
| | | CGAAAGCCTGAAGGC |
| SEQ ID | GI 440918690 NM_001272072 Homo sapiens | GGAGACCGCCACCTT |
| NO: 4179 | amine oxidase, copper containing 1 | CCTGGACACTTTCCA |
| | (AOC1), transcript variant 1, mRNA | CTACTATGATGCCGA |
| | | TGACCCGGTCCATTA |
| SEQ ID | GI 189339218 NG_007955 Homo sapiens | TGCCTGGAGCTACAG |
| NO: 4180 | cytochrome P450 family | ATGACCGTTGCCCCA |
| | 17 subfamily A member 1 | CTCAACCAGGAAAAG |
| | (CYP17A1), RefSeqGene on chromosome [symbol] | AAGGCACTGCATTTA |
| SEQ ID | GI 1024846744 NG_047021 Homo sapiens | GAGAGCAAGCCATTC |
| NO: 4181 | nudix hydrolase 15 | AGGTGACTGATGCTG |
| | (NUDT15), RefSeqGene on chromosome 13 | CTCTGATCCATCCAT |
| | | TGGTGTCCTCTGGGC |
| SEQ ID | GI 297139734 NG_021330 Homo sapiens | TGACAAGAGCTGGGA |
| NO: 4182 | serum amyloid A1 | ACTAACGTGCCTCAA |
| | (SAA1), RefSeqGene on chromosome 11 | GCCCCCACAAGGACC |
| | | ACAGCATTTTCTCAT |
| SEQ ID | GI 237858805 NG_012139 Homo sapiens | GCCCTTTTTAGGGTC |
| NO: 4183 | uncoupling protein 1 | TCTGGCCACCAGAGC |
| | (UCP1), RefSeqGene on chromosome 4 | CAGCTTTACTCACCT |
| | | GTGCATGGCATTCTA |
| SEQ ID | GI 209862753 NM_004343 Homo sapiens | ATGCACGGAGACTCA |
| NO: 4184 | calreticulin | GAATACAACATCATG |
| | (CALR), mRNA | TTTGGTCCCGACATC |
| | | TGTGGCCCTGGCACC |
| SEQ ID | GI 54144649 NM_002090 Homo sapiens | ATGACAGGGTGGGGA |
| NO: 4185 | C-X-C motif chemokine ligand 3 | ACTGGAGGGTGGGGG |
| | (CXCL3), mRNA | GATTGAAATGCAAGC |
| | | AATTAGTGGATCACT |
| SEQ ID | GI 807201065 NM_001008397 Homo sapiens | GGGCTGAAGGAACTG |
| NO: 4186 | glutathione peroxidase 8 | CACAAAGAGTTTGGA |
| | (putative) | CCATCCCACTTCAGC |
| | (GPX8), transcript variant 1, mRNA | GTGTTGGCTTTTCCC |
| SEQ ID | GI 153791376 NM_024310 Homo sapiens | AGAAGGCCAAGCCGC |
| NO: 4187 | pleckstrin homology and | GCATCTTCTTCCTCT |
| | FYVE domain containing 1 | TTAACGACATCCTGG |
| | (PLEKHF1), mRNA | TGTATGGCAGCATCG |
| SEQ ID | GI 282721038 NG_016269 Homo sapiens | CCTGTCTTTGTCTCT |
| NO: 4188 | adenylate kinase 2 | GATGGGCTTTGGGAA |
| | (AK2), RefSeqGene on chromosome 1 | CAGACAGCATCCTGG |
| | | GCAGAATGGTCCCAT |
| SEQ ID | GI 974987493 NG_012830 Homo sapiens | ACAGTGGAAGTTTGA |
| NO: 4189 | DEAD-box helicase 3, X-linked | ACAGTGGAGACTGTG |
| | (DDX3X), RefSeqGene on chromosome X | GGAGCCCAGTGCCCA |
| | | GAGCAACCTACTCTA |
| SEQ ID | GI 224809272 NG_011478 Homo sapiens | GTGTGTCAAAGCTAA |
| NO: 4190 | uncoupling protein 2 | ACAGGCTGGGTGGGC |
| | (UCP2), RefSeqGene on chromosome 11 | ATGAGCAAGTCAGCT |
| | | GGTTCATTCAGCAGC |
| SEQ ID | GI 164698262 AK314515 Homo sapiens | GCCGGGAGAAACAAG |
| NO: 4191 | cDNA, FLI95334, Homo sapiens | CACGGACGCAAACAC |
| | protein inhibitor of activated STAT, 1 | GAACTTCTCACAAAA |
| | (PIAS1), mRNA | GCCCTGCATTTGCTA |
| SEQ ID | GI 527317415 NM_000304 Homo sapiens | CACCAAACGAATGGC |
| NO: 4192 | peripheral myelin protein 22 | TGCAGTCTGTCCAGG |
| | (PMP22), transcript variant 1, mRNA | CCACCATGATCCTGT |
| | | CGATCATCTTCAGCA |
| SEQ ID | GI 42519913 NM_012448 Homo sapiens | ACAAGCTCAGCAGCT |
| NO: 4193 | signal transducer and | CCAAGGAGAAGCCCT |
| | activator of transcription 5B | TCATCAGATGCAAGC |
| | (STAT5B), mRNA | GTTATATGGCCAGCA |
| SEQ ID | GI 282721038 NG_016269 Homo sapiens | TGTGGAACAGGGAAA |
| NO: 4194 | adenylate kinase 2 | GCTGATGCCTGTGTT |
| | (AK2), RefSeqGene on chromosome 1 | TTGGTCAACCCCATT |
| | | CTCTCCTGGCACTCT |
| SEQ ID | GI 269954683 NG_015990 Homo sapiens | ACCCGAAAGAACCGC |
| NO: 4195 | C-C motif chemokine ligand 5 | CAAGTGTGTGCCAAC |
| | (CCL5), RefSeqGene on chromosome 17 | CCAGAGAAGAAATGG |
| | | GTTCGGGAGTACATC |
| SEQ ID | GI 398303864 NG_032876 Homo sapiens | TTCCTCACCACAGAG |
| NO: 4196 | major histocompatibility | GTTCCTGAGGTCACA |
| | complex, class II, DQ alpha 1 | GTGTTTTCCAAGTCT |
| | (HLA-DQA1), RefSeqGene on chromos [symbol] | CCCGTGACACTGGGT |
| SEQ ID | GI 429535871 NG_033022 Homo sapiens | TCCCTTCCTGCCCCT |
| NO: 4197 | interleukin 12A | CCTCAGAAGCTTCAT |
| | (IL12A), RefSeqGene on chromosome 3 | GCCCTTCTCACCATG |
| | | TCTCACCTACTGATG |
| SEQ ID | GI 1145140176 NG_008729 Homo sapiens | GGGAAGAATGAGCTG |
| NO: 4198 | superoxide dismutase 2 | GGCCATTGTGAAGGG |
| | (SOD2), RefSeqGene on chromosome 6 | AAACTCCAGGAAGCT |
| | | GGGTCTTCACTGAGG |
| SEQ ID | GI 584277093 NM_001675 Homo sapiens | TGGCTTCTGATTCTC |
| NO: 4199 | activating transcription factor 4 | ATTCAGGCTTCTCAC |
| | (ATF4), transcript variant 1, mRNA | GGCATTCAGCAGCAG |
| | | CGTTGCTGTAACCGA |
| SEQ ID | GI 205277450 NM_001135054 Homo sapiens | GGGGCCTTCACCTGC |
| NO: 4200 | single Ig and TIR domain containing | TCCATCCAGAACATC |
| | (SIGIRR), transcript variant 1, mRNA | AGCTTCTCCTCCTTC |
| | | ACTCTTCAGAGAGCT |
| SEQ ID | GI 1036032775 AH003000 Homo sapiens | ACAACCCGTCTTTCA |
| NO: 4201 | steroid 5-alpha-reductase | GAAAAGCGGCGTGAC |
| | (SRD5A1) gene, complete cds | AGGGAAAACAGCGAA |
| | | CAGCTCTAAGGGGAA |
| SEQ ID | GI 326806980 NG_028248 Homo sapiens | CCTGTAGGTCTTTGC |
| NO: 4202 | aryl hydrocarbon receptor | CTGCTGAGGGCAGGG |
| | nuclear translocator | GTTTGTGTAATGGTT |
| | (ARNT), RefSeqGene on chromosome 1 | ATGGTTCCGGACAGG |
| SEQ ID | GI 219521823 NG_009222 Homo sapiens | GGGGCCGTTTAGCCT |
| NO: 4203 | ELK1, ETS transcription factor | AATGGTGAAATGCTT |
| | (ELK1), RefSeqGene on chromosome X | AGTCAACTGGAACCT |
| | | GACTGCCTGGCTCCA |
| SEQ ID | GI 1028224162 NG_047195 Homo sapiens | ATACAGAGCAGCAGA |
| NO: 4204 | transducin beta like 1 | GCAAGGCAGTTCTGT |
| | X-linked receptor 1 | GAAGTTTGTCCCTGG |
| | (TBL1XR1), RefSeqGene on chromosome 3 | CAGCCCTTAGCAGTG |
| SEQ ID | GI 49574519 NM_052972 Homo sapiens | GCTGGGACCCCGTTT |
| NO: 4205 | leucine rich alpha-2-glycoprotein 1 | CATCCATCCATGCTT |
| | (LRG1), mRNA | CCTAGAACACACGAT |
| | | GGGCTTTCCTTACCC |
| SEQ ID | GI 296841050 NM_138733 Homo sapiens | GTCACCACCGACCCA |
| NO: 4206 | phosphoglycerate kinase 2 | GCCCCTCAACAGCAA |
| | (PGK2), mRNA | GTTGGTTCTTCAGCA |
| | | TTAAGATCCAGGTGT |
| SEQ ID | GI 89142743 NM_023068 Homo sapiens | ACCAGACAGTGACAC |
| NO: 4207 | sialic acid binding Ig like lectin 1 | TAGTCTGCAACACAC |
| | (SIGLEC1), mRNA | CCAATGAGGCACCCA |
| | | GTGATCTCCGCTACA |
| SEQ ID | GI 94717603 DQ508420 Homo sapiens | ATGTTTTTGGGTTGG |
| NO: 4208 | caspase 5, apoptosis- | TCCAGAAAAGCCTCT |
| | related cysteine peptidase | GTGGGGAGAGAACAC |
| | (CASP5) gene, complete cds | AGGAGTTGGACGGCA |
| SEQ ID | GI 1015635417 AH006868 Homo sapiens | TGCCCAATGAAAAGT |
| NO: 4209 | CD9 antigen | ATGGTCAGCGAGCGA |
| | (CD9) gene, complete cds | AGGTTTGCAAGGAGA |
| | | CAGACGAGGGCGAAA |
| SEQ ID | GI 1213447 U48869 Human | CAACCGGCGGCGGCT |
| NO: 4210 | cdk-inhibitor p57/KIP2 | GCCGCAGAGCAGCGT |
| | (CDKNIC) gene, complete cds | TCGGTTTTGTTTTTA |
| | | AATTTTGAAAACTGT |
| SEQ ID | GI 237874266 NG_012190 Homo sapiens | CTGGGGAAGTGACCA |
| NO: 4211 | anti-Mullerian hormone | GGGTGGAAGATAGAG |
| | (AMH), RefSeqGene on | ACTTGTGTCCATAGG |
| | chromosome 19 | GTTCAGGAAGGCGGG |
| SEQ ID | GI 33946335 NM_002166 Homo sapiens | AACCCCTGTGGACGA |
| NO: 4212 | inhibitor of DNA binding 2, HLH protein | CCCGATGAGCCTGCT |
| | (ID2), mRNA | ATACAACATGAACGA |
| | | CTGCTACTCCAAGCT |
| SEQ ID | GI 78190493 NM_013434 Homo sapiens | GAATGAGTGTCCCAC |
| NO: 4213 | potassium voltage-gated | GGGCCTGGTGGACGA |
| | channel interacting protein 3 | AGACACCTTCAAACT |
| | (KCNIP3), transcript variant 1, mRNA | CATTTACGCGCAGTT |
| SEQ ID | GI 77744386 DQ230907 Homo sapiens | CACAGACGTTAGCCC |
| NO: 4214 | nudix | CCGTTCGTGTTTTTG |
| | (nucleoside diphosphate | TTCTGAGAAAGGTAG |
| | linked moiety X)-type motif 1 | GCCAGCTTGCCTGGA |
| | (NUDT1) gene, complete cds | |
| SEQ ID | GI 224809239 NG_011444 Homo sapiens | GCTCTCCCTATGTTG |
| NO: 4215 | fatty acid binding protein 2 | CTTAGGCTGGCCTCA |
| | (FABP2), RefSeqGene on chromosome 4 | AACTGCTGGTTTCGA |
| | | ACTCCTGGCCTGAAG |
| SEQ ID | GI 308199420 NG_027788 Homo sapiens | GGCAGCACTTCCCAA |
| NO: 4216 | MX dynamin like GTPase 1 | CCTTCCTGACATGGG |
| | (MX1), RefSeqGene on chromosome 21 | CGTTTCTGTGAACTC |
| | | CAGTGTGATGGAGAA |
| SEQ ID | GI 37675281 NM_020664 Homo sapiens | CGTGGACCAGGCTCT |
| NO: 4217 | 2,4-dienoyl-CoA reductase 2 | GAAGGAGTTTGGCAG |
| | (DECR2), mRNA | AATCGACATTCTCAT |
| | | TAACTGTGCGGCCGG |
| SEQ ID | GI 148227403 NM_005292 Homo sapiens | CGTCTGCTTTATGCC |
| NO: 4218 | G protein-coupled receptor 18 | CTTCCACATCTGTTT |
| | (GPR18), transcript variant 1, mRNA | CGCTTTCCTGATGCT |
| | | GGGAACGGGGGAGAA |
| SEQ ID | GI 20160210 AY094608 Homo sapiens | AAGACCGAACTGGTT |
| NO: 4219 | cyclin-dependent kinase inhibitor 2C | TCGCTGTCATTCATG |
| | (CDKN2C) gene, complete cds | ATGCGGCCAGAGCAG |
| | | GTTTCCTGGACACTT |
| SEQ ID | GI 283837929 NG_016244 Homo sapiens | ACTCAACCCAGACAG |
| NO: 4220 | toll like receptor 5 | ATGGTTAGCTCCCCG |
| | (TLR5), RefSeqGene on chromosome 1 | ACAGGACTTAAGCAA |
| | | GTGGGCCAGTCTCTG |
| SEQ ID | GI 210032511 NG_008871 Homo sapiens | TCACTTCCTGGTGGG |
| NO: 4221 | xanthine dehydrogenase | CGTTAGACACATACC |
| | (XDH), RefSeqGene on chromosome 2 | CTGCTGAGCAGATCA |
| | | GCTAGGAAAAGCCCT |
| SEQ ID | GI 57863305 NM_052872 Homo sapiens | GAAATGCCACTCCTT |
| NO: 4222 | interleukin 17F | ACCCAGTGCTCTGCA |
| | (IL17F), mRNA | ACAAGTCCTGTCTGA |
| | | CCCCCAATTCCCTCC |
| SEQ ID | GI 528078360 NM_003955 Homo sapiens | TCCTCAGCTTCCCCT |
| NO: 4223 | suppressor of cytokine signaling 3 | CTGCCTCCAAACAGG |
| | (SOCS3), mRNA | GGACACTTCGGGAAT |
| | | GCTGAACTAATGAGA |
| SEQ ID | GI 313661479 NM_145725 Homo sapiens | TACTTTGGGGGAATG |
| NO: 4224 | TNF receptor associated factor 3 | AGTATCCTTGGTTCT |
| | (TRAF3), transcript variant 1, mRNA | GCCCTGTGAGGGAGT |
| | | CGTGTGTCCCTGCTC |
| SEQ ID | GI 45238842 AY563103 Homo sapiens | TCTGTCGCCGTTTGT |
| NO: 4225 | interleukin 2 receptor, alpha | CCTGGGTGAGGTCAT |
| | (IL2RA) gene, complete cds | TCTGGTCATCTGTGG |
| | | CAGGAGGACTACAAA |
| SEQ ID | GI 344925888 NG_029692 Homo sapiens | GAGGCAGACATCTGG |
| NO: 4226 | 2′-5′-oligoadenylate synthetase 2 | GAAAAGTCCAGCTGG |
| | (OAS2), RefSeqGene on chromosome 12 | GGCAAGCATTTGAAG |
| | | TCTGCCTTCCTACCA |
| SEQ ID | GI 574997036 NG_033896 Homo sapiens | GGATCTGAGGAGATC |
| NO: 4227 | tubulin beta 4A class IVa | TCAGGAGCACAGAGT |
| | (TUBB4A), RefSeqGene on chromosome 19 | GGGTGCTTGGTGGCA |
| | | GAGGTACTGCTGTTC |
| SEQ ID | GI 815890844 NM_001836 Homo sapiens | GCAGCAAAGTGTGTG |
| NO: 4228 | chymase 1 | CCACTCATTCTGGTC |
| | (CMA1), transcript variant 1, mRNA | TACCCTTGGTCCCTC |
| | | AGCCACAACCCTAAG |
| SEQ ID | GI 67515420 DQ088846 Homo sapiens | TGGATGGGAGAACAG |
| NO: 4229 | myeloperoxidase | CTTAAAAGCCATCCT |
| | (MPO) gene, complete cds | TCCCAAAGCCTTGCC |
| | | TCTGTCTGGACACCC |
| SEQ ID | GI 527498317 NG_008179 Homo sapiens | TAGCATCTCTGTCCT |
| NO: 4230 | general transcription factor Ili | GTGTTGAGTTGTGGT |
| | (GTF2I), RefSeqGene on chromosome 7 | TGCCCGTCACTGGGC |
| | | TGTAAAGTGCCTTGC |
| SEQ ID | GI 156105692 NM_001037335 Homo sapiens | GGGCCCCATCTCACC |
| NO: 4231 | helicase with zinc finger 2 | AACCAACTATCGGCA |
| | (HELZ2), transcript variant 1, mRNA | GAGGATGCACCAGTT |
| | | TCTCTATGAGGAGGA |
| SEQ ID | GI 117606543 EF064746 Homo sapiens | CTGCAAGGCCCACCT |
| NO: 4232 | complement component | TCTAGTCGGTGTGTA |
| | (3d/Epstein Barr virus) receptor 2 | ATTGCTGGACAGGGA |
| | (CR2) gene, complete cds | GTTGCTTGGACCAAA |
| SEQ ID | GI 68165954 DQ086801 Homo sapiens | GAAGAGCTCCGCGGA |
| NO: 4233 | suppressor of cytokine signaling 1 | GTACAGAGCCCATTT |
| | (SOCS1) gene, complete cds | TCTAGCTGTGTCCAC |
| | | TGAGGCTGAACGGAT |
| SEQ ID | GI 284055256 NG_016403 Homo sapiens | TGGCCTGCAACTGTA |
| NO: 4234 | dystrophin related protein 2 | CCCTCTGGAGTGGGA |
| | (DRP2), RefSeqGene on chromosome X | AGCAGGCAGAAAGCC |
| | | CTATACTTAACTGGA |
| SEQ ID | GI 308522770 NM_005469 Homo sapiens | CTGGACGAGGATCTC |
| NO: 4235 | acyl-CoA thioesterase 8 | TTCAGAGGAAGGCAT |
| | (ACOT8), mRNA | TACTGGGTACCGGCC |
| | | AAGAGGCTGTTTGGT |
| SEQ ID | GI 117606573 EF064761 Homo sapiens | TGGCTCCATTGGCTC |
| NO: 4236 | chemokine | AGGTGGTTTAGTCAT |
| | (C-C motif) ligand 28 | TGGGAACCAGCATCA |
| | (CCL28) gene, complete cds | TCTGTGGCACTCCAG |
| SEQ ID | GI 20514390 AF507942 Homo sapiens | ATGGAGTGGCTGCCA |
| NO: 4237 | cyclin-dependent kinase 4 | TGGAAGGAAGAAAAG |
| | (CDK4) gene, complete cds | CTGCCATTTCCCTTC |
| | | TGGACACTGAGAGGG |
| SEQ ID | GI 62632868 AY995114 Homo sapiens | TTCACCGCCTCACCT |
| NO: 4238 | tumor necrosis factor receptor | ACTAAATCAGGCTCT |
| | 1-associated death domain protein | CCGGAAAGGGTAAGG |
| | (TRADD) gene, complete cds [symbol] | CCCAGGAATCTGCAT |
| SEQ ID | GI 395393980 NG_011966 Homo sapiens | CAGACCGGCTCTAAA |
| NO: 4239 | mitogen-activated protein | GGCCTAAGGGGAGGA |
| | kinase kinase kinase 7 | CTGGTGTAAGTCCTG |
| | (MAP3K7), RefSeqGene on chromosome 6 | AAGTCCAAAGGCCAA |
| SEQ ID | GI 584277161 NG_008225 Homo sapiens | ACGCCTCTGCAACTC |
| NO: 4240 | POU class 1 homeobox 1 | TGCCTCTGATAATGC |
| | (POU1F1), RefSeqGene on chromosome 3 | ATCACAGTGCTGCCG |
| | | AGTGTCTACCAGTCT |
| SEQ ID | GI 42794751 NM_004457 Homo sapiens | AAGGACTGGGGAAAC |
| NO: 4241 | acyl-CoA synthetase long chain | AGCTGTAACATTTGC |
| | family member 3 | CACCCTCAGAAGCTG |
| | (ACSL3), transcript variant 1, mRNA | CTGGTCCTGTGTCAC |
| SEQ ID | GI 392050748 NM_001267774 Homo sapiens | CGGCTTGACCAATGA |
| NO: 4242 | intraflagellar transport 20 | CCTATGTCACAAGAG |
| | (IFT20), transcript variant 1, mRNA | GTGGCGTGTAAGGAA |
| | | TGCAGCCCCCTGAAG |
| SEQ ID | GI 170560906 EU567146 Homo sapiens | CCCCAGAGCTAAATC |
| NO: 4243 | solute carrier family 22 | AAGTACATCCCAGCC |
| | (organic anion transporter), member 6 | CAAAGGCCCCTCCCA |
| | (SLC22A6) gene, complete cds | ACACTCATCATTGCA |
| SEQ ID | GI 110735399 NM_001031680 Homo sapiens | TAGGCAGTCCTTCTG |
| NO: 4244 | runt related transcription factor 3 | GTGATCCTATTCCAT |
| | (RUNX3), transcript variant 1, mRNA | TCCCTCCTGCTGCGG |
| | | TTTCTCTTGGCCTGT |
| SEQ ID | GI 29825390 AY242132 Homo sapiens | AGGCCTTTGCTTTCT |
| NO: 4245 | cannabinoid receptor 2 | GCTCCATGCTGTGCC |
| | (CNR2) gene, complete cds | TCATCAACTCCATGG |
| | | TCAACCCTGTCATCT |
| SEQ ID | GI 306816 M96233 Human glutathione | GACCTCCTGAGGGAT |
| NO: 4246 | transferase class mu number 4 | TTGGGGGAGGATGGG |
| | (GSTM4) gene, complete cds | GATTTGACAGAAAGA |
| | | GGCCAGAACTGGAGA |
| SEQ ID | GI 300244555 NG_023331 Homo sapiens | GTAGGAGCCTCTCTC |
| NO: 4247 | matrix Gia protein | CCTACTGCTGCTACA |
| | (MGP), RefSeqGene on chromosome 12 | CAAGACCCTGAGACT |
| | | GACCTGCAGGACGAA |
| SEQ ID | GI 262331544 NG_013350 Homo sapiens | TGAAACCAGACAGGA |
| NO: 4248 | nuclear receptor subfamily | ACCAAGCCATCTGCT |
| | 3 group C member 2 | CCCGGAACAGTGCTC |
| | (NR3C2), RefSeqGene on chromosome 4 | TGCCATTGCACTAAG |
| SEQ ID | GI 117606579 EF064764 Homo sapiens | GCTGAGTGAACAGGG |
| NO: 4249 | chemokine | TGCCACCAGAGTGTG |
| | (C-C motif) ligand 22 | GGTATGGCAGTAGGT |
| | (CCL22) gene, complete cds | AGTCAGTAGCTGTGG |
| SEQ ID | GI 27368251 AY196903 Homo sapiens | AACAGAGCTCCTTGG |
| NO: 4250 | interleukin 1 receptor antagonist | ACCCTTCCCTCTAAG |
| | (ILIRN) gene, complete cds | GTCTCTGGAAATGGG |
| | | AAACCCTGCAGAGGG |
| SEQ ID | GI 30526191 AY275469 Homo sapiens | CTTTGCTACCCTCGC |
| NO: 4251 | chemokine C motif receptor 1 | CACCACTGTCCTATA |
| | (XCR1) gene, complete cds | CTGCCTGGTGTTTCT |
| | | CCTCAGCCTAGTGGG |
| SEQ ID | GI 189217849 NG_007932 Homo sapiens | AATGGGTGGAGACAT |
| NO: 4252 | cytochrome P450 family 4 | TCCCAAGTGCCTGTC |
| | subfamily A member 11 | CTCATTGGCTATGGG |
| | (CYP4A11), RefSeqGene | GAGGCAAAGTTCGTG |
| | on chromosome [symbol] | |
| SEQ ID | GI 324711016 NG_012837 Homo sapiens | CACATGACAACTTCC |
| NO: 4253 | GC, vitamin D binding protein | CACCCAGTCCTCTGC |
| | (GC), RefSeqGene on chromosome 4 | TTTGTTCTCCAGGCC |
| | | ACTTAGCAAGCCCAT |
| SEQ ID | GI 374088072 NG_032029 Homo sapiens | TTTGCAGAGCTGAGC |
| NO: 4254 | MAP kinase interacting | TAGCCAGCAAAGTCG |
| | serine/threonine kinase 1 | GGTTCTCAGTGGGGA |
| | (MKNK1), RefSeqGene on chromosome 1 | GAGGATCATCTCTGG |
| SEQ ID | GI 260593656 NG_013016 Homo sapiens | TTCTTCCTGGAGCTC |
| NO: 4255 | phosphoglycerate mutase 2 | TTTCTGTTGGTGCAC |
| | (PGAM2), RefSeqGene on chromosome 7 | TTGAGGTTTCAAGCC |
| | | GCCTGTGTGTCCAGG |
| SEQ ID | GI 353249887 NG_030361 Homo sapiens | CCATGCCAGGCCTCC |
| NO: 4256 | Spl transcription factor | AGACCATTAACCTCA |
| | (SP1), RefSeqGene on chromosome 12 | GTGCATTGGGTACTT |
| | | CAGGAATCCAGGTGC |
| SEQ ID | GI 257153325 NG_012807 Homo sapiens | ACATCCTCAAGTAAG |
| NO: 4257 | ST3 beta-galactoside alpha-2,3- | TACCGCCCCTGGGAC |
| | sialyltransferase 5 | TGTAGATCCCCCTGC |
| | (ST3GAL5), RefSeqGene on chromosome [symbol] | TTTGGGTCAACTGTG |
| SEQ ID | GI 748585173 NM_198580 Homo sapiens | TGAAAGCTTCCGGAA |
| NO: 4258 | solute carrier family 27 member 1 | TTGACTGTGACCACT |
| | (SLC27A1), mRNA | TGGATGTCACCACTG |
| | | TCAGCCCCTGCCTTG |
| SEQ ID | GI 27501923 AY204752 Homo sapiens | TGAGCCAGGATTCCT |
| NO: 4259 | excision repair cross-complementing | GCTTCTGAGCCAGGC |
| | rodent repair deficiency, | AGTGGTCAGAGTCCC |
| | complementation group 6 | ATAAACTGTGTGTCT |
| | (ERCC | |
| SEQ ID | GI 189458805 NG_007981 Homo sapiens | GTCCTCGTTCCAGTT |
| NO: 4260 | ATP binding cassette subfamily A member 1 | TCACTGATGACCTCT |
| | (ABCA1), RefSeqGene on chromosome 9 | CGACCAGCTCCCTCA |
| | | CAGCAGTCTTTGCCA |
| SEQ ID | GI 429535873 NG_033024 Homo sapiens | GCCTGCCTGTGTCAT |
| NO: 4261 | colony stimulating factor 2 | GGCCTCAGCCCTGAT |
| | (CSF2), RefSeqGene on chromosome 5 | GTCATCTTTCCATGA |
| | | GAAAGATGTGTGGCT |
| SEQ ID | GI 570700862 NG_033874 Homo sapiens | ATTGGGTGTTTCCTG |
| NO: 4262 | nuclear factor kappa B subunit 2 | CAGCTCCAGGGGTTG |
| | (NFKB2), RefSeqGene on chromosome 10 | CTGAGATAAGGAATA |
| | | CAAAGCCCCCAGCTT |
| SEQ ID | GI 224809368 NG_011523 Homo sapiens | ATCCCTCCCCATCAC |
| NO: 4263 | TIR domain containing adaptor protein | AAGGTTCTTAATCAC |
| | (TIRAP), RefSeqGene on chromosome 11 | AGCTGCCGAATCCCC |
| | | TTTTGCCACGGAAGG |
| SEQ ID | GI 49574517 NM_003695 Homo sapiens | TCTCGCTTCTGCAAG |
| NO: 4264 | lymphocyte antigen 6 family member D | ACCACGAACACAGTG |
| | (LY6D), mRNA | GAGCCTCTGAGGGGG |
| | | AATCTGGTGAAGAAG |
| SEQ ID | GI 751368105 NM_145911 Homo sapiens | AATATTTGCGGGCGT |
| NO: 4265 | zinc finger protein 23 | GAAGCGGCCCGAGGT |
| | (ZNF23), transcript variant 1, mRNA | GATACCACAAGTCTT |
| | | TTGACATGCTGAGGT |
| SEQ ID | GI 154482086 EU048234 Homo sapiens | ACCTTGTGCCTTGGA |
| NO: 4266 | CCAAT/enhancer binding protein | AATGCAAACTCACCG |
| | (C/EBP), alpha | CTCCAATGCCTACTG |
| | (CEBPA) gene, complete cds | AGTAGGGGGAGCAAA |
| SEQ ID | GI 262205923 NM_013444 Homo sapiens | AGTACCCCAGGCATG |
| NO: 4267 | ubiquilin 2 | CAGAGCCTGCTGCAA |
| | (UBQLN2), mRNA | CAGATAACTGAAAAC |
| | | CCCCAGCTGATTCAG |
| SEQ ID | GI 648216382 NG_029573 Homo sapiens | TCTTGACAGGTAGGT |
| NO: 4268 | TNF receptor superfamily member 8 | GGCAAAGGCTCTGGA |
| | (TNFRSF8), RefSeqGene on chromosome 1 | AGGGCCAGAACATCA |
| | | CTGCAGCCATGTTTT |
| SEQ ID | GI 411147377 NM_014339 Homo sapiens | TGGCTTTTCTTCCTG |
| NO: 4269 | interleukin 17 receptor A | ACTGCAATGTGGCAT |
| | (IL17RA), transcript variant 1, mRNA | TGTGCCAGCTACCTC |
| | | CTCTTTCTCGGCCTC |
| SEQ ID | GI 387849236 NM_002613 Homo sapiens | GTAGTTGGGGTCCAT |
| NO: 4270 | 3-phosphoinositide dependent protein kinase 1 | TGATTGTGCAGGGGA |
| | (PDPK1), transcript variant 1, mRNA | ACGTGCAGGAGGTTT |
| | | TTCTAGGCACCGTGT |
| SEQ ID | GI 306518675 NG_027688 Homo sapiens | ACACTCACACCCACA |
| NO: 4271 | CD4 molecule | CTCTCACCCCATGTA |
| | (CD4), RefSeqGene on chromosome 12 | CTCACACCCATGCAC |
| | | TCACACACTGTCACA |
| SEQ ID | GI 1147544507 NG_028250 Homo sapiens | AGCCACCTGCACTAA |
| NO: 4272 | PPARG coactivator 1 alpha | CCAGTTCATGGACTG |
| | (PPARGCIA), RefSeqGene on chromosome 4 | AATGGCCAAGCAGTG |
| | | CATGCAGCCAAAAAG |
| SEQ ID | GI 342349297 NG_029616 Homo sapiens | TGGGAGGGAGAGTCA |
| NO: 4273 | protein kinase C zeta | GTCAGTGATGTGTTT |
| | (PRKCZ), RefSeqGene on chromosome 1 | GAGCTGCTGTGTCCA |
| | | CCTGGAAACCATCGT |
| SEQ ID | GI 227908773 NG_011948 Homo sapiens | CAGGAGTTGGCCCCT |
| NO: 4274 | triosephosphate isomerase 1 | CTCCTTTTGCTACAA |
| | (TPI1), RefSeqGene on chromosome 12 | ATCCTTGCCTTGCAA |
| | | AGGGGAGGTGAGGAT |
| SEQ ID | GI 564730481 NM_001287262 Homo sapiens | GGAAGGTGGCTGAGA |
| NO: 4275 | butyrophilin like 10 | GCCACCTGCCCGGTG |
| | (BTNL10), mRNA | AGTCCTCTATTCAGT |
| | | TCTGTCCTCACAATC |
| SEQ ID | GI 18653447 AF478696 Homo sapiens | TGAACCAAACAGGAA |
| NO: 4276 | coagulation factor II | CCCAAACCCCCAGCT |
| | (thrombin) | GCTCTGACACCAAGG |
| | (F2) gene, complete cds | ACTTCCCTAAGCATG |
| SEQ ID | GI 224809231 NG_011436 Homo sapiens | GACATTTCCTCTGCT |
| NO: 4277 | alpha 2-HS glycoprotein | GCCAAACCCATGGCA |
| | (AHSG), RefSeqGene on chromosome 3 | CTCTGAGGCTGACTG |
| | | TGTCCACCTCATTCC |
| SEQ ID | GI 315506987 NG_028041 Homo sapiens | TGAGGGGTAACCTTT |
| NO: 4278 | adenylate kinase 3 | CCTCCAGTGAATAGA |
| | (AK3), RefSeqGene on chromosome 9 | AGCCCAGAGCACCAC |
| | | TCGGATGGAGCCAAC |
| SEQ ID | GI 301500676 NG_023437 Homo sapiens | AGGTCAGGAGGCAGT |
| NO: 4279 | apolipoprotein L4 | AAGGTGGCTTGAGAG |
| | (APOL4), RefSeqGene on chromosome 22 | TTTGTGTGCAGCCAG |
| | | GACTAATAGCCAGGT |
| SEQ ID | GI 225637493 NG_011688 Homo sapiens | CATCATCCCTCTAGG |
| NO: 4280 | growth hormone receptor | AATGGGCCTGCCTTG |
| | (GHR), RefSeqGene on chromosome 5 | GTGTCCTTGTCATGC |
| | | AAAGTCACTGGCTGG |
| SEQ ID | GI 342187223 NG_029593 Homo sapiens | GGCACCAGAAGGTAT |
| NO: 4281 | LYN proto-oncogene, | GAAGGGCCTTGGAGT |
| | Src family tyrosine kinase | ACTGGGCTTTCTTAT |
| | (LYN), RefSeqGene on chromosome 8 | GGCTGCGAGAGAGGA |
| SEQ ID | GI 42794751 NM_004457 Homo sapiens | GCTTGCTAGGGGGAA |
| NO: 4282 | acyl-CoA synthetase | ATATTCGTCTCCTGT |
| | long chain family member 3 | TGTGTGGTGGCGCTC |
| | (ACSL3), transcript variant 1, mRNA | CACTTTCTGCAACCA |
| SEQ ID | GI 18034675 AF458059 Homo sapiens | GACCCCTCAACAGCA |
| NO: 4283 | IL25 mRNA, complete cds | GGGCCATCTCCCCCT |
| | | GGAGATATGAGTTGG |
| | | ACAGAGACTTGAACC |
| SEQ ID | GI 748585188 NM_004040 Homo sapiens | GAAGCACTTCTGTCC |
| NO: 4284 | ras homolog family member B | CAATGTGCCCATCAT |
| | (RHOB), mRNA | CCTGGTGGCCAACAA |
| | | AAAAGACCTGCGCAG |
| SEQ ID | GI 312434026 NM_002995 Homo sapiens | ACCACAGGAAGTGCA |
| NO: 4285 | X-C motif chemokine ligand 1 | CTGACCACTGGAGGC |
| | (XCL1), mRNA | ATAAAAGAGGTCCTC |
| | | AAAGAGCCCGATCCT |
| SEQ ID | GI 38045925 AY449709 Homo sapiens | ATGGATTCTGGGAAT |
| NO: 4286 | interleukin 7 receptor | GGCCAGAAAAACAAA |
| | (IL7R) gene, complete cds | TGCCTGTGGTTGTGA |
| | | GCAGTGCCCACACCC |
| SEQ ID | GI 166235119 NG_007149 Homo sapiens | ACTTGTCATGGTGAA |
| NO: 4287 | B-cell CLL/lymphoma 6 | ATGGCAAATCTGGGC |
| | (BCL6), RefSeqGene on chromosome 3 | TTCCTGTGGTTCCAG |
| | | AAGCCTGCGACTGGA |
| SEQ ID | GI 225543208 NG_011640 Homo sapiens | ATCCATGCCCACCTT |
| NO: 4288 | interleukin 6 | TGGCATGAGCTGAGG |
| | (IL6), RefSeqGene on chromosome 7 | GTTATTGCTTCTCAG |
| | | GGCTTCCTTTTCCCT |
| SEQ ID | GI 209447076 NG_008726 Homo sapiens | AAGGACGTCCATTGG |
| NO: 4289 | paraoxonase 3 | AAACGCCGGACTTGG |
| | (PON3), RefSeqGene on chromosome 7 | GAGAGAGGATCAGGC |
| | | TACCAGTTGGATGTT |
| SEQ ID | GI 1003088850 NM_007368 Homo sapiens | TGACCCGGCCCTGTA |
| NO: 4290 | RAS p21 protein activator 3 | GCTACAGCAAGAAGT |
| | (RASA3), transcript variant 1, mRNA | CCCACTTTGACTTTG |
| | | AGGAGGAAGACGTGG |
| SEQ ID | GI 81301989 DQ217942 Homo sapiens | GCAACAGAATTGGCA |
| NO: 4291 | EGF-like module containing | TCCTCCAGGCACTTG |
| | mucin-like hormone receptor-like 1 | TTAGAAATGCAAAAT |
| | (EMR1) gene, complete cds | CCCCGGCCCCCTTCC |
| SEQ ID | GI 20336662 AF503510 Homo sapiens | GGTGAGAGACAAAGG |
| NO: 4292 | coagulation factor X | AGACACAGGCAGAGA |
| | (F10) gene, complete cds | GATGTGGACAGCACA |
| | | GCCCACAGACACACA |
| SEQ ID | GI 117606489 EF064719 Homo sapiens | CTGCCCACACTTGTT |
| NO: 4293 | interleukin 27 receptor, alpha | TCCTGGCCTAAGGGC |
| | (IL27RA) gene, complete cds | TTTTTCTGGTCACTG |
| | | AGGCATTTTGCCTGC |
| SEQ ID | GI 345525588 NG_029933 Homo sapiens | TGCTCATCTACATCA |
| NO: 4294 | interleukin 16 | GGCTCAGCAAAGCAG |
| | (IL16), RefSeqGene on chromosome 15 | GCTCCTAGAACCAAG |
| | | GAAGTCCCTCCCCAC |
| SEQ ID | GI 256985178 NG_012796 Homo sapiens | CAAAAGGATCAACAT |
| NO: 4295 | glycogen phosphorylase L | GGCCCATCTCTGCAT |
| | (PYGL), RefSeqGene on chromosome 14 | TGTCGGTTCCCATGC |
| | | TGTGAATGGCGTGGC |
| SEQ ID | GI 193290120 NG_008160 Homo sapiens | GGAGAGATTCAGGGT |
| NO: 4296 | transaldolase 1 | AGAGCTTCGAGCTGA |
| | (TALD01), RefSeqGene on chromosome 11 | CTTCCACGTGGCCAG |
| | | TTTGTGGTCAGCGAG |
| SEQ ID | GI 46488017 AY601851 Homo sapiens | GTGAGTCAGCAGAAG |
| NO: 4297 | mutS homolog 2, colon | TGTCCATTGTGGACT |
| | cancer, nonpolyposis type 1 | GCATCTTAGCCCGAG |
| | (E. coli) | TAGGGGCTGGTGACA |
| | (MSH2) gene, complete cds | |
| SEQ ID | GI 68165954 DQ086801 Homo sapiens | TGGGTCTCCTTGTAT |
| NO: 4298 | suppressor of cytokine signaling 1 | TCCATCACCCTCCAC |
| | (SOCS1) gene, complete cds | AGAGTGTGGCCTCCA |
| | | GCAGATGTTCAGTGG |
| SEQ ID | GI 83026500 DQ309025 Homo sapiens | TCTTGGGGCCTCTAT |
| NO: 4299 | transforming growth factor | TTATCTGTCAGGGTC |
| | beta 1 induced transcript 1 | TCCGATGGGAGGGTC |
| | (TGFB1I1) gene, complete cds | TCCGAACCTGAGGTC |
| SEQ ID | GI 188219645 NG_007730 Homo sapiens | TCTCCAAAGCTGCCG |
| NO: 4300 | protein tyrosine phosphatase, | ATTGGTGACTGAATG |
| | receptor type C | CAGCTTTGGACCATG |
| | (PTPRC), RefSeqGene on chromosome 1 | CCCTGACACTGCATA |
| SEQ ID | GI 224494751 NM_138801 Homo sapiens | GCCAACCGAATCGCC |
| NO: 4301 | galactose mutarotase | AAAGGAACCTTCAAG |
| | (GALM), mRNA | GTGGATGGGAAGGAG |
| | | TATCACCTGGCCATT |
| SEQ ID | GI 982742415 NM_020530 Homo sapiens | AACCTGTTCCCCTCT |
| NO: 4302 | oncostatin M | GCTACTAGCCACTGA |
| | (OSM), transcript variant 1, mRNA | GAAGTGCACTTTAAG |
| | | AGGTGGGAGCTGGGC |
| SEQ ID | GI 1024846760 NG_046988 Homo sapiens | TGGAGGGGAAGATCA |
| NO: 4303 | E2F transcription factor 1 | GTCTGGTTTTGGTCC |
| | (E2F1), RefSeqGene on chromosome 20 | GGGTACTTTTGAGCA |
| | | GCCTGTGAGACCTCC |
| SEQ ID | GI 307746866 NG_027761 Homo sapiens | ATGGCAAGGAACCAT |
| NO: 4304 | heat shock protein family A | CCCGTGGCATAAACC |
| | (Hsp70) member 5 | CAGATGAAGCTGTAG |
| | (HSPA5), RefSeqGene on chromosome 9 | CGTATGGTGCTGCTG |
| SEQ ID | GI 323668281 NG_023034 Homo sapiens | GGGATTTGTGGGAGT |
| NO: 4305 | vascular cell adhesion molecule 1 | CTTGTAGTCCCAGCG |
| | (VCAM1), RefSeqGene on chromosome 1 | TTGTGTAGGAGTGGC |
| | | CTCACTGTGCTCTGT |
| SEQ ID | GI 19923714 NM_138284 Homo sapiens | GCCTTTCCATGGAGA |
| NO: 4306 | interleukin 17D | CTCGTAAGCAGCTTC |
| | (IL17D), mRNA | ATCTGACACGGGCAT |
| | | CCCTGGCTTGCTTTT |
| SEQ ID | GI 71274112 NM_005946 Homo sapiens | AGCTGCTGTGCCTGA |
| NO: 4307 | metallothionein 1A | TGTCCGGACAGCCCT |
| | (MT1A), mRNA | GCTCGAAGATATAGA |
| | | AAGAGTGACCTGCAC |
| SEQ ID | GI 1015809708 NM_003807 Homo sapiens | CAGATGAGGAACCTG |
| NO: 4308 | TNF superfamily member 14 | GACCCAGTTGGCTCA |
| | (TNFSF14), transcript variant 1, mRNA | AGTCCATGCAGGAAA |
| | | TGTGGGGGAAACCAG |
| SEQ ID | GI 188219645 NG_007730 Homo sapiens | CCCTTTCACCCTGGT |
| NO: 4309 | protein tyrosine phosphatase, | GTTGGCCTCCATCAG |
| | receptor type C | CTGTGTCTGGATTCT |
| | (PTPRC), RefSeqGene on chromosome 1 | GACAGTCTCTCCTGT |
| SEQ ID | GI 254939589 NG_012569 Homo sapiens | TCCCACGAACACCAC |
| NO: 4310 | toll like receptor 7 | GAACCTCACCCTCAC |
| | (TLR7), RefSeqGene on chromosome X | CATTAACCACATACC |
| | | AGACATCTCCCCAGC |
| SEQ ID | GI 95104789 NM_005860 Homo sapiens | GGAACAAGATCAACC |
| NO: 4311 | follistatin like 3 | TCCTCGGCTTCTTGG |
| | (FSTL3), mRNA | GCCTTGTCCACTGCC |
| | | TTCCCTGCAAAGATT |
| SEQ ID | GI 974987493 NG_012830 Homo sapiens | ACTCCTGCCAACCTT |
| NO: 4312 | DEAD-box helicase 3, X-linked | CCCTCATCCACTGTG |
| | (DDX3X), RefSeqGene on chromosome X | CTCCTATCATACCAG |
| | | CCGCCTTTCAATCCT |
| SEQ ID | GI 324711016 NG_012837 Homo sapiens | GTAGGTGAGGGCAGG |
| NO: 4313 | GC, vitamin D binding protein | AGAAGAAAGGCTGTC |
| | (GC), RefSeqGene on chromosome 4 | ATCCTGCCCATCTTG |
| | | GGAAGATAGCCCTCC |
| SEQ ID | GI 224809277 NG_011481 Homo sapiens | TGGGCTTGGCTGTTT |
| NO: 4314 | interleukin 18 receptor accessory protein | TCTGTGCACTGAGGT |
| | (IL18RAP), RefSeqGene on chromosome 2 | CAAGTGGAATTGCTT |
| | | GCTGTGCTTTGCGTT |
| SEQ ID | GI 343962600 NG_029791 Homo sapiens | ACCTTTGCCCCTGAC |
| NO: 4315 | TNF receptor superfamily member 1B | TTTCTCCATGGACCA |
| | (TNFRSF1B), RefSeqGene on chromosome 1 | CGGACAAGTGACTTG |
| | | ATCTCCCTGAGCTGT |
| SEQ ID | GI 345199309 NM_002167 Homo sapiens | ATCCCTTCCTGGAGA |
| NO: 4316 | inhibitor of DNA binding 3, HLH protein | CTAAACCTGGTGCTC |
| | (ID3), mRNA | AGGAGCGAAGGACTG |
| | | TGAACTTGTGGCCTG |
| SEQ ID | GI 4885488 NM_005372 Homo sapiens | ACCGCGGTGTTCCTG |
| NO: 4317 | MOS proto-oncogene, serine/threonine kinase | TGGCCATAAAGCAAG |
| | (MOS), mRNA | TGAACAAGTGCACCA |
| | | AGAACCGACTAGCAT |
| SEQ ID | GI 18653445 AF479264 Homo sapiens | ACAAGGAGTGGCATG |
| NO: 4318 | ligase IV, DNA, ATP-dependent | AGTGCATGACCCTGG |
| | (LIG4) gene, complete cds | ACGACCTAGAACAAC |
| | | TTAGGGGGAAGGCAT |
| SEQ ID | GI 401782607 NG_032906 Homo sapiens | AGCATGAGCCCCGAA |
| NO: 4319 | coagulation factor II thrombin receptor | GGGCAGAACTGTGAG |
| | (F2R), RefSeqGene on chromosome 5 | TCAAAACCATGTGTG |
| | | TCATGGAATGCTGGT |
| SEQ ID | GI 405778361 NG_032938 Homo sapiens | GCAAAGGAATAGACT |
| NO: 4320 | semaphorin 3B | TGCAGAGCTAGCAAT |
| | (SEMA3B), RefSeqGene on chromosome 3 | GAAAAGGGGGCCTGG |
| | | ACTGTCAGGGGCCAG |
| SEQ ID | GI 169646347 NM_001115152 Homo sapiens | AGCATGCTGGGGACC |
| NO: 4321 | CD300 molecule like family member d | GTCCTCTGGGTAAAC |
| | (CD300LD), mRNA | AGACCACAAAGAAGG |
| | | TCTTGAAGGAGGAGG |
| SEQ ID | GI 208431777 NM_001135638 Homo sapiens | GGAGCCAGGCGTTCA |
| NO: 4322 | phosphatidylinositol-4- | CCTTGGTCGTCCTGA |
| | phosphate 5-kinase type 1 alpha | TGTTTTACCTCAGAC |
| | (PIP5K1A), transcript variant 1, [symbol] | TCCACCTTTGGAGGA |
| SEQ ID | GI 1049010865 AH002678 Homo sapiens | TGCTTCCTCTTCCTT |
| NO: 4323 | isolate Patient with P-450(C21) | TGAAGCATCATCAGT |
| | deficiency membrane protein | AGTCACACCCTCACA |
| | (CD3E) gene, complete cds | GCTGGCCTGCCCTCT |
| SEQ ID | GI 224809232 NG_011437 Homo sapiens | AAGCCTTCAAAGGCC |
| NO: 4324 | arachidonate 5-lipoxygenase | AGGCAGCCACCGTAA |
| | (ALOX5), RefSeqGene on chromosome 10 | TAAGAGAAAGCCACT |
| | | GCTTCTGCCATGACA |
| SEQ ID | GI 193794854 NG_008178 Homo sapiens | CCAGCTCCTGGTACT |
| NO: 4325 | glycerol kinase | TCCTCAGGTATTGGT |
| | (GK), RefSeqGene on chromosome X | GCTCTGGCTATTGCT |
| | | CACTTCCCAGCCTCC |
| SEQ ID | GI 297632354 NG_007470 Homo sapiens | AAGCTCTGCTTCTGG |
| NO: 4326 | glycophorin A | AAGAGCAAGGGCTCC |
| | (MNS blood group) | CCTCTCCCTTTCATG |
| | (GYPA), RefSeqGene on chromosome 4 | CAGTTTCTAGGAGCA |
| SEQ ID | GI 674651085 NG_012882 Homo sapiens | AAGCTCAAGGGCATG |
| NO: 4327 | toll like receptor 8 | CCAGGCAAAGGCCTT |
| | (TLR8), RefSeqGene on chromosome X | TTTTCTGGCAGCTTG |
| | | AACTTGTCTCAGCAG |
| SEQ ID | GI 214832245 NM_017853 Homo sapiens | ATGACCTGGGATGCC |
| NO: 4328 | thioredoxin like 4B | TAACTGTCTACTCCC |
| | (TXNL4B), transcript variant 1, mRNA | TGCAAGCCTCAGAGC |
| | | AGCCAAGTCATTGGT |
| SEQ ID | GI 1025608653 NG_047027 Homo sapiens | TTCCGTGATTTGCTT |
| NO: 4329 | Jun proto-oncogene, AP-1 | GCTTCACACTGTGGT |
| | transcription factor subunit | AGCCTGGCCACGAAA |
| | (JUN), RefSeqGene on chromosome | GAACCAGGATTCCCG |
| SEQ ID | GI 684179348 NG_034249 Homo sapiens | TCGTGTGATGTGTGT |
| NO: 4330 | transmembrane protein 173 | GCTGCCTTTGGCTGA |
| | (TMEM173), RefSeqGene on chromosome 5 | GTGTGTGGAGTCCTG |
| | | CTCAGGTGTTAGGTA |
| SEQ ID | GI 23510408 NM_003057 Homo sapiens | AATGCTGAGCTGTAC |
| NO: 4331 | solute carrier family 22 member 1 | CCCACATTCGTCAGG |
| | (SLC22A1), transcript variant 1, mRNA | AACCTCGGAGTGATG |
| | | GTGTGTTCCTCCCTG |
| SEQ ID | GI 83699680 DQ314887 Homo sapiens | GGGATGAGCTCAGGG |
| NO: 4332 | v-rel reticuloendotheliosis viral | TTTAGGCACACAGCT |
| | oncogene homolog B, nuclear | CAGGATGTATACGGA |
| | factor of kappa light polypeptide [symbol] | GGCTACACTGCTGCC |
| SEQ ID | GI 974987490 NG_016453 Homo sapiens | TCAGCCACAGAGAGG |
| NO: 4333 | transient receptor potential | GGGCCGAATGGGTTG |
| | cation channel subfamily M member 1 | CTGTTTTTGCAGTTG |
| | (TRPM1), RefSeqGene Or [symbol] | AATGCAAACGCTTTT |
| SEQ ID | GI 282721038 NG_016269 Homo sapiens | ACCATGCGTAAGAGC |
| NO: 4334 | adenylate kinase 2 | AGTTTGTCTGCAGTG |
| | (AK2), RefSeqGene on chromosome 1 | GTGCAGGACGTAAAC |
| | | ACAGGGCACTTGTGG |
| SEQ ID | GI 300388175 NG_011471 Homo sapiens | ACGTCACTTACTCCC |
| NO: 4335 | purinergic receptor P2X 7 | CACTCTGTCATCCTT |
| | (P2RX7), RefSeqGene on chromosome 12 | CTATCTGCAGTATCC |
| | | CACCCGCAGGACGCT |
| SEQ ID | GI 166706752 EU332835 Homo sapiens | TATCACCGAGGACAG |
| NO: 4336 | v-akt murine thymoma viral | CTGCTCTGATTCTGA |
| | oncogene homolog l | AGTGTGAACGCCTGC |
| | (AKT1) gene, complete cds | GTACGGCTGATGCTG |
| SEQ ID | GI 50234986 AY675319 Homo sapiens | ATTATGAGCCTCCAT |
| NO: 4337 | transforming growth factor, | TTGGTTCCAAGGTGC |
| | beta receptor II | GGGAGCACCCCTGTG |
| | (70/80 kDa)(TGFBR2) gene, complete cds | TCGAAAGCATGAAGG |
| SEQ ID | GI 86278451 DQ374395 Homo sapiens | ATTTTGCATCTCAGG |
| NO: 4338 | UDP glucuronosyltransferase | TCACCCGATGACCCG |
| | 1 family, polypeptide A1 | TGCCTTTATCACCCA |
| | (UGT1A1) gene, complete cds | TGCTGGTTCCCATGG |
| SEQ ID | GI 209447076 NG_008726 Homo sapiens | CAGCCCTAGGAAACA |
| NO: 4339 | paraoxonase 3 | CATGCAGTGAGGGAG |
| | (PON3), RefSeqGene on chromosome 7 | GTACAGCAAAGGCGT |
| | | CATTAAGCCAACCGC |
| SEQ ID | GI 807045929 NG_029892 Homo sapiens | AATCACCATGGCCAA |
| NO: 4340 | potassium voltage-gated | CCCTCAATGCAAAGA |
| | channel subfamily J member 6 | GGATGAGAAATGTGG |
| | (KCNJ6), RefSeqGene on chromosor [symbol] | TCCAGCCCTGGGAGG |
| SEQ ID | GI 193290120 NG_008160 Homo sapiens | TCAGAAGTTCTGGAG |
| NO: 4341 | transaldolase 1 | CCCAGACTTGTGACG |
| | (TALD01), RefSeqGene on chromosome 11 | GGTGGGAATGGAGAA |
| | | TGGTTGGTTGGGACT |
| SEQ ID | GI 364023822 NM_130898 Homo sapiens | TGGCTTTGCTTCCCA |
| NO: 4342 | cAMP responsive element | CTGGGATTCCTACTT |
| | binding protein 3 like 4 | AGGTGTCTGCCCTCA |
| | (CREB3L4), transcript variant 1, mRNA | GGGGTCCAAATCACT |
| SEQ ID | GI 930697465 NM_005564 Homo sapiens | TCATCCGCTTCTCCA |
| NO: 4343 | lipocalin 2 | AATCTCTGGGCCTCC |
| | (LCN2), mRNA | CTGAAAACCACATCG |
| | | TCTTCCCTGTCCCAA |
| SEQ ID | GI 227908772 NG_011947 Homo sapiens | AGGATGAGGGCACAC |
| NO: 4344 | tryptophan hydroxylase 1 | AAGGAAAGACAAGGT |
| | (TPH1), RefSeqGene on chromosome 11 | GGTGCTTGGTGTGTC |
| | | TGGGGAAACAGCAAG |
| SEQ ID | GI 315434256 NM_001926 Homo sapiens | CCAAGGCTGAGCCAC |
| NO: 4345 | defensin alpha 6 | TCCAAGCTGAGGATG |
| | (DEFA6), mRNA | ATCCACTGCAGGCAA |
| | | AAGCTTATGAGGCTG |
| SEQ ID | GI 54112075 AY771350 Homo sapiens | AATCTCCAGTGGCAT |
| NO: 4346 | heme oxygenase | CTGCAATGTGGGTAT |
| | (decycling) 2 | TCTAGCCTCCAGTGC |
| | (HMOX2) gene, complete cds | TCACGTGGCTCACCT |
| SEQ ID | GI 352962130 NG_030351 Homo sapiens | AGGTTGGACAGAGGC |
| NO: 4347 | eukaryotic translation | AGTTGGGAGTTCAGG |
| | initiation factor 2 alpha kinase 2 | AGAGAGTCCATGGTG |
| | (EIF2AK2), RefSeqGene on chromoso [symbol] | GAAGCGTGCTTTTGA |
| SEQ ID | GI 538917457 NM_005896 Homo sapiens | GGAGACGTCCACCAA |
| NO: 4348 | isocitrate dehydrogenase | TCCCATTGCTTCCAT |
| | (NADP(+)) 1, cytosolic | TTTTGCCTGGACCAG |
| | (IDH1), transcript variant 1, mRNA | AGGGTTAGCCCACAG |
| SEQ ID | GI 50345287 AY682848 Homo sapiens | GCCAATGACGACACA |
| NO: 4349 | receptor(TNFRSF)- | GACCAGCAAAGCAGA |
| | interacting serine-threonine kinase 1 | ACCGAGACTGTCCGC |
| | (RIPK1) gene, complete cds | ACTTGGTTCGGAAAC |
| SEQ ID | GI 1154491910 NG_052982 Homo sapiens | ACCCACACAGAGTGT |
| NO: 4350 | ubiquitin conjugating enzyme E2 G2 | CATGGGTGACAGTTT |
| | (UBE2G2), RefSeqGene on chromosome 21 | CTGGTTGAAGTAGCT |
| | | CCACGCCTGGCTTCT |
| SEQ ID | GI 436408720 NM_014226 Homo sapiens | GGAACAAATGGAAGA |
| NO: 4351 | MOK protein kinase | GTGCCGGTGCTGAGA |
| | (MOK), transcript variant 1, mRNA | CCCTTGAAGTGCATC |
| | | CCTGCGAGCAAGAAG |
| SEQ ID | GI 21634442 AF274025 Homo sapiens | GACCTATGCTGTGGT |
| NO: 4352 | GTP-binding protein Rab25 mRNA, | GGAGCGATGGCTGAA |
| | complete cds | GGAGCTCTATGACCA |
| | | TGCTGAAGCCACGAT |
| SEQ ID | GI 11691883 AB034990 Homo sapiens | CTCGTAAGGAGCACA |
| NO: 4353 | HERPUD1 gene for stress | GCCTAGTTCCCTTGC |
| | protein Herp, complete cds | GTGCACAGTTCACAA |
| | | TAGGGTTCGGCTCCT |
| SEQ ID | GI 313747578 NM_015162 Homo sapiens | AAACCAGCTCACTGA |
| NO: 4354 | acyl-CoA synthetase | CTGACAGGCAGCCAC |
| | bubblegum family member 1 | TCTCCAAAGAGTCCC |
| | (ACSBG1), transcript variant 1, mRNA | TGAACCATGCTCTCG |
| SEQ ID | GI 94538326 NM_002038 Homo sapiens | TGCCCTGATGGGCTA |
| NO: 4355 | interferon alpha inducible protein 6 | CGCCACCCACAAGTA |
| | (IFI6), transcript variant 1, mRNA | TCTCGATAGTGAGGA |
| | | GGATGAGGAGTAGCC |
| SEQ ID | GI 802084027 NM_006236 Homo sapiens | CACACCTCCACCTCC |
| NO: 4356 | POU class 3 homeobox 3 | ACCAATGCACTCTTC |
| | (POU3F3), mRNA | TTCCTCCTCCTTCTC |
| | | CAGACAACTGCTGGG |
| SEQ ID | GI 571026712 NG_033872 Homo sapiens | ATGAACAGCCAAGTG |
| NO: 4357 | cytochrome cl | TTAAGGTGTTGAACC |
| | (CYC1), RefSeqGene on chromosome 8 | ACTTGGCCCCCATTC |
| | | TGATGGCAGCAGGGA |
| SEQ ID | GI 238018065 NG_012195 Homo sapiens | AATGGGCACAGGGGA |
| NO: 4358 | fatty acid amide hydrolase | AGCAGAGATATAGGT |
| | (FAAH), RefSeqGene on chromosome 1 | GAGGACAGATCAAGG |
| | | TGGACTTGGGCAGCC |
| SEQ ID | GI 238028972 NG_011992 Homo sapiens | ACAAGGAAGGCACAT |
| NO: 4359 | nitric oxide synthase 3 | CCTGGCTGACCAAGA |
| | (NOS3), RefSeqGene on chromosome 7 | GGTTAGACTGTGCTC |
| | | GGGCACTGACAAGAA |
| SEQ ID | GI 574584717 NM_001289177 Homo sapiens | TGTCGGAAGGCTGCA |
| NO: 4360 | acyl-CoA synthetas | AAATCCTTGATCAAG |
| | e bubblegum family member 2 | CTGGGTTTGGAGCGT |
| | (ACSBG2), transcript variant 1, mRNA [symbol] | TTCCACGGAGTTGGT |
| SEQ ID | GI 728797213 LN680437 Homo sapiens | ATCCTCGCTGTGGTT |
| NO: 4361 | mRNA for CD47 | GGACTGAGTCTCTGT |
| | | ATTGCGGCGTGTATA |
| | | CCAATGCATGGCCCT |
| SEQ ID | GI 325495520 HQ692855 Homo sapiens | CAGCACCTTCATGGA |
| NO: 4362 | nerve growth factor | CGGCTACACAGGAGA |
| | IB nuclear receptor variant 1 | GTTTGACACCTTCCT |
| | (NR4A1) mRNA, complete cds | CTACCAGCTGCCAGG |
| SEQ ID | GI 61743979 NM_017564 Homo sapiens | TGTTTCTGTGGGTGA |
| NO: 4363 | stabilin 2 | GAGATGTGTTGCTGT |
| | (STAB2), mRNA | GCCCACCCAGTACAG |
| | | CTTCCTCCTCTGACC |
| SEQ ID | GI 1026191091 NG_047061 Homo sapiens | TCCCGCCTGTTTTCT |
| NO: 4364 | marker of proliferation Ki-67 | TTCTGACTCTATCTG |
| | (MKI67), RefSeqGene on chromosome 10 | GCAGCCCGATGCCAC |
| | | CCAGTACAGGAAGTG |
| SEQ ID | GI 238776809 NG_012246 Homo sapiens | CCTGGGACGTGACTT |
| NO: 4365 | N-acetyltransferase 2 | TCGAAAGTGCTTCTC |
| | (NAT2), RefSeqGene on chromosome 8 | AGCATCCATTCCCCC |
| | | CTCCCCTCCACTTAG |
| SEQ ID | GI 1142736558 NG_052842 Homo sapiens | TCCTCCAAGCATCTC |
| NO: 4366 | pyruvate dehydrogenase | TAGCACAGAGACCCT |
| | phosphatase regulatory subunit | GTTTTCTCCCCTTGT |
| | (PDPR), RefSeqGene on chromos [symbol] | AATGGGCCAGTGCCC |
| SEQ ID | GI 238814317 NG_012266 Homo sapiens | CGGAGCTGGGTATCT |
| NO: 4367 | X-box binding protein 1 | CAAATCTGCTTTCAT |
| | (XBP1), RefSeqGene on chromosome 22 | CCAGCCACTGCCCAA |
| | | AGCCATCTTCCTGCC |
| SEQ ID | GI 70995210 NM_001398 Homo sapiens | ATGTGGCCCGGATCA |
| NO: 4368 | enoyl-CoA hydratase 1 | GCTGGTACCTCCGTG |
| | (ECH1), mRNA | ACATCATCACTCGAT |
| | | ACCAGGAGACCTTCA |
| SEQ ID | GI 908212441 NG_042288 Homo sapiens | AGGATGGCAGGTTTG |
| NO: 4369 | methylsterol monooxygenase 1 | GGCAGAGAGAAAAGT |
| | (MSM01), RefSeqGene on chromosome 4 | TGGGTGGCAGTGCAG |
| | | TCATCACACAACCAA |
| SEQ ID | GI 1047600468 NM_001718 Homo sapiens | GCGCGGAGTTCGTGA |
| NO: 4370 | bone morphogenetic protein 6 | GCGAGAAGGAAGTTA |
| | (BMP6), mRNA | AACCTCGCGGAATAG |
| | | ACTGGCATTTCGGGA |
| SEQ ID | GI 195976780 NM_005201 Homo sapiens | AAATGCCTAGGGAGA |
| NO: 4371 | C-C motif chemokine receptor 8 | GCTGTGAAAAGTCAT |
| | (CCR8), mRNA | CATCCTGCCAGCAGC |
| | | ACTCCTCCCGTTCCT |
| SEQ ID | GI 574957130 NM_001815 Homo sapiens | TGGTGTGTTTCCTGC |
| NO: 4372 | carcinoembryonic antigen | TCCTTGCCAAAACTG |
| | related cell adhesion molecule 3 | GAAGAACCAGCATCC |
| | (CEACAM3), transcript variant 1 [symbol] | AGCGTGACCTCAAGG |
| SEQ ID | GI 1008909437 NG_023257 Homo sapiens | AGGGCCCAAGGTCTC |
| NO: 4373 | lactotransferrin | CCATCTGCCAAGCTA |
| | (LTF), RefSeqGene on chromosome 3 | TTGTCCAAGCGTGAG |
| | | TTGATGTCTCAGGAA |
| SEQ ID | GI 195927007 NM_052813 Homo sapiens | AGTCAGGCCTGACTC |
| NO: 4374 | caspase recruitment domain family member 9 | AGCTGCTGATGACTG |
| | (CARD9), transcript variant 1, mRNA | AGGTCATGAAGCTGC |
| | | AGAAGAAGGTGCAGG |
| SEQ ID | GI 292494880 NG_007148 Homo sapiens | CTCCTGCTTTCTCAC |
| NO: 4375 | ATPase phospholipid transporting 8B1 | TGAGACTGTGCAGAT |
| | (ATP8B1), RefSeqGene on chromosome 18 | TGTGTGGGTGGCGAG |
| | | TTGGTTGTGCAAGGC |
| SEQ ID | GI 327180724 NG_028256 Homo sapiens | TAGAGGGCATCGTGA |
| NO: 4376 | mitogen-activated protein kinase kinase 3 | GGCTCAGAGAGGTTA |
| | (MAP2K3), RefSeqGene on chromosome 17 | AGTGACTTGCTGAGG |
| | | GTCACACAGCAGGGA |
| SEQ ID | GI 211904121 NG_008930 Homo sapiens | TAGGCCAGGGTGGAG |
| NO: 4377 | cytochrome p450 oxidoreductase | GCACTGTTTTCAAGA |
| | (POR), RefSeqGene on chromosome 7 | ACAGGAGTGTTCCCA |
| | | CAGGTGACCCTTTCT |
| SEQ ID | GI 1128611453 NM_019058 Homo sapiens | AGGCAGCCACCTAAG |
| NO: 4378 | DNA damage inducible transcript 4 | GTGGAGGTGGGGGAA |
| | (DDIT4), mRNA | TAGTGTTTCCCAGGA |
| | | AGCTCATTGAGTTGT |
| SEQ ID | GI 57790279 AY865620 Homo sapiens | TCAGAGGACACCTGA |
| NO: 4379 | signal transducer and | GTCCGTGAACACCTT |
| | activator of transcription 1, 91 kDa | TGCAAAGCCTTCTTG |
| | (STAT1) gene, complete cds | GGGTGTGAGGGAAGT |
| SEQ ID | GI 927028873 NM_004836 Homo sapiens | AATGGACCAGGATGA |
| NO: 4380 | eukaryotic translation | GGAAGAGCAGACGGT |
| | initiation factor 2 alpha kinase 3 | TCTGACCCCAATGCC |
| | (EIF2AK3), transcript | AGCTTATGCCAGACA |
| | variant 1, mRN [symbol] | |
| SEQ ID | GI 1015637181 AH000040 Homo sapiens | TCCATGTGCCCCCTC |
| NO: 4381 | hematopoietic stem cell antigen | TACCATTTGCAGAGT |
| | (CD34) gene, complete cds | CCTGCACAGTTTTCT |
| | | GGCTGGAGCCTAGAA |
| SEQ ID | GI 18653447 AF478696 Homo sapiens | AGTGCGCTCATTACA |
| NO: 4382 | coagulation factor II | GCCTTACAGTAACCA |
| | (thrombin) | GGTGGGGGGTAAGGT |
| | (F2) gene, complete cds | CCTGTGCCCATTTCA |
| SEQ ID | GI 45505189 AY572796 Homo sapiens | TGTAGTGACAGCACC |
| NO: 4383 | signal transducer and | ACTGCCTCTGTTTCC |
| | activator of transcription 3 | CTTCCTCTTGTGATG |
| | (acute-phase response factor) | GCAGCAGCGTCTCAA |
| | (STAT3) gene, [symbol] | |
| SEQ ID | GI 189217867 NG_007938 Homo sapiens | AGGTTTACCCCTGGA |
| NO: 4384 | cytochrome P450 family 3 | GGGAAAGTCACAAGC |
| | subfamily A member 5 | TCATGTCCAGAAGGC |
| | (CYP3A5), RefSeqGene on chromosome 7 | CAGTAGCAGCAAGCT |
| SEQ ID | GI 381140032 NG_032168 Homo sapiens | GTGCCCCATCAGTTC |
| NO: 4385 | glycerol-3-phosphate dehydrogenase 1 | ATCGGCAAGATCTGT |
| | (GPD1), RefSeqGene on chromosome 12 | GACCAGCTCAAGGGC |
| | | CATCTGAAGGCAAAC |
| SEQ ID | GI 325995139 NG_008854 Homo sapiens | AGCTTGGGGACCTCA |
| NO: 4386 | potassium voltage-gated | GGATCTTGCCCCAAG |
| | channel subfamily Q member 3 | TCACAGAACTAAGGA |
| | (KCNQ3), RefSeqGene on chromos [symbol] | ATTGGCAGAGCCAGT |
| SEQ ID | GI 1139349906 NG_052802 Homo sapiens | GTGACCATTTGGACC |
| NO: 4387 | NLR family pyrin domain containing 4 | GCCTTTTTGCTCCCA |
| | (NLRP4), RefSeqGene on chromosome 19 | AGGAAGCTGGGAAAC |
| | | AGCCACGTACAGTGA |
| SEQ ID | GI 341865607 NG_029555 Homo sapiens | ATGGCTGCCATCTGT |
| NO: 4388 | ETS proto-oncogene 1, | TAGGACAATCATGAC |
| | transcription factor | ACACATACTGAGGCT |
| | (ETS1), RefSeqGene on chromosome 11 | GCCCCGTCTCCTGGG |
| SEQ ID | GI 1013417860 NG_046847 Homo sapiens | TAGCATGAACGCAGC |
| NO: 4389 | ubiquitin specific peptidase 7 | CACAGACAACAATTG |
| | (USP7), RefSeqGene on chromosome 16 | AGTGGCGAGGCTGTC |
| | | CCCACAAAGCTTTGT |
| SEQ ID | GI 78190493 NM_013434 Homo sapiens | TCCATGGCAGGAAGG |
| NO: 4390 | potassium voltage-gated | CGGGGGATTTCAAGT |
| | channel interacting protein 3 | TTAGGGATTGGGTCG |
| | (KCNIP3), transcript variant 1, mRNA | TGGTGGAGAATCTGA |
| SEQ ID | GI 478431032 NM_003693 Homo sapiens | ATGTTGTACCCATCT |
| NO: 4391 | scavenger receptor class F member 1 | CCAGGCCACCAGAAC |
| | (SCARF1), transcript variant 1, mRNA | CCTGATGACCTTGAA |
| | | TTTGGGGAGTGGGGA |
| SEQ ID | GI 83699680 DQ314887 Homo sapiens | CGAGGCTTCTGCCAT |
| NO: 4392 | v-rel reticuloendotheliosis viral | TTTCAGTTCGCCAAC |
| | oncogene homolog B, nuclear | TCATGGTGCAAGGTG |
| | factor of kappa light | GCTGTTGTAGCTCCA |
| | polypeptide [symbol] | |
| SEQ ID | GI 342187247 NG_029422 Homo sapiens | GTGGGGACAGGGAAG |
| NO: 4393 | major histocompatibility | GTGGCTGTTGGACAG |
| | complex, class 1, C | GAGTTGTCAATGTGT |
| | (HLA-C), RefSeqGene on chromosome 6 | GCCTTGTTGCAAAGA |
| SEQ ID | GI 167860125 NM_002639 Homo sapiens | TGGTTGGCACTAGAC |
| NO: 4394 | serpin family B member 5 | TGGTGGCAGGGGCTT |
| | (SERPINB5), mRNA | CTAGCTGACTTGCAC |
| | | AGGGATTCTCACAAT |
| SEQ ID | GI 769468216 NM_003246 Homo sapiens | GACCCACGAATGATG |
| NO: 4395 | thrombospondin 1 | CCTTGGGTGGGCAAC |
| | (THBS1), mRNA | ACGATTGCATGTTGT |
| | | GGAGACACTTCGGAA |
| SEQ ID | GI 341865606 NG_029554 Homo sapiens | AGGAATTTGAACACA |
| NO: 4396 | Fc fragment of IgE receptor II | GGTAGTGTGGGTCAG |
| | (FCER2), RefSeqGene on chromosome 19 | GCATCTGCAGCCCTA |
| | | GCCACTATGCCACCT |
| SEQ ID | GI 1049010865 AH002678 Homo sapiens | GTGATGTCGGTGGCC |
| NO: 4397 | isolate Patient with P-450(C21) | ACAATTGTCATAGTG |
| | deficiency membrane protein | GACATCTGCATCACT |
| | (CD3E) gene, complete cds | GGGGGCTTGCTGCTG |
| SEQ ID | GI 684179348 NG_034249 Homo sapiens | ACTTCCCACCCCAAG |
| NO: 4398 | transmembrane protein 173 | GGAACTCAAAGCTTG |
| | (TMEM173), RefSeqGene on chromosome 5 | TGTCAAGGCCAAGGA |
| | | AAAATTCCCCCTCCG |
| SEQ ID | GI 1021312320 NM_001323565 Homo sapiens | GTTCGACCAGTACAG |
| NO: 4399 | cysteine dioxygenase type 1 | TCGTGGTCGTGGTTT |
| | (CD01), transcript variant 1, mRNA | GCAGTTTGTTGTTGG |
| | | TGGTGGCAGTGGTGG |
| SEQ ID | GI 16118554 AF398885 Homo sapiens | AGACATCTCGATCCT |
| NO: 4400 | ELMOl mRNA, complete cds | GCAGCGGTCCTTGGC |
| | | CATTTTGGAGTCGAT |
| | | GGTGCTCAATAGCCA |
| SEQ ID | GI 164696344 AK313967 Homo sapiens | TGCCGACCTGAAAAT |
| NO: 4401 | cDNA, FLI94614, Homo sapiens | GGAGAGGAGCACACC |
| | interleukin 18 receptor 1 | TTTGCTGTGGAGATT |
| | (IL18R1), mRNA | TTGCCCAGGGTGTTG |
| SEQ ID | GI 49574519 NM_052972 Homo sapiens | AGCGACCAAAAAGCC |
| NO: 4402 | leucine rich alpha-2-glycoprotein 1 | CAGGGGGCATTCAAC |
| | (LRG1), mRNA | CCCATGTTTCTAGAA |
| | | CTCTGTTCCTGCTGC |
| SEQ ID | GI 11545840 NM_022120 Homo sapiens | GGGGGACACATCCAA |
| NO: 4403 | 3-oxoacid CoA-transferase 2 | CTAACCATGCTTGGA |
| | (OXCT2), mRNA | GCCATGCAGGTTTCC |
| | | AAATACGGCGACCTG |
| SEQ ID | GI 22128580 AF533019 Homo sapiens | GCTGGCGTCTGCATA |
| NO: 4404 | growth arrest and DNA- | CGAGAGACTTGGTTG |
| | damage-inducible, beta | AACTTGGTTGGTCCT |
| | (GADD45B) gene, complete cds | TGTCTGCACCCTCGA |
| SEQ ID | GI 238018096 NG_012212 Homo sapiens | AAGCTCCCGAGTCTT |
| NO: 4405 | C-C motif chemokine ligand 11 | TTCCCCACATTCAAG |
| | (CCL11), RefSeqGene on chromosome 17 | AGGGTTGCTGGGTGC |
| | | ATCCATACCCAGCTA |
| SEQ ID | GI 1028908926 NG_050619 Homo sapiens | TTGGGAGGGGAGAGA |
| NO: 4406 | fatty acid binding protein 7 | GAAGGGCAGGGAATA |
| | (FABP7), RefSeqGene on chromosome 6 | TCAAGTCGGATTCTC |
| | | CTGCTCTGTCCCCAG |
| SEQ ID | GI 520975386 NM_001278651 Homo sapiens | TGGCAGTGCAGGATG |
| NO: 4407 | Ran GTPase activating protein 1 | CAGTAGATGCCCTGA |
| | (RANGAP1), transcript variant 1, mRNA | TGCAGAAGGCTTTCA |
| | | ACTCCTCGTCCTTCA |
| SEQ ID | GI 1068937310 NG_008847 Homo sapiens | ACAGCTTTAACCTCC |
| NO: 4408 | glucokinase | AGCCTTGTTTCTTCA |
| | (GCK), RefSeqGene on chromosome 7 | TGGTTGCAGGGTGGC |
| | | TATCACAGCGCTGGC |
| SEQ ID | GI 291084831 NG_016755 Homo sapiens | TCAGGGCACCTTTCA |
| NO: 4409 | succinate-CoA ligase alpha subunit | CAGCCAGCAGGCATT |
| | (SUCLG1), RefSeqGene on chromosome 2 | GGAATATGGCACCAA |
| | | ACTCGTTGGAGGAAC |
| SEQ ID | GI 974005408 NM_015133 Homo sapiens | tGCACCAGCATCTACA |
| NO: 4410 | mitogen-activated protein | GGACGTGGACATTGA |
| | kinase 8 interacting protein 3 | GCCCTACGTCAGCAA |
| | (MAPK8IP3), transcript variant 1, i | GATGCTAGGCACTGG |
| SEQ ID | GI 954170001 NM_002620 Homo sapiens | CCACTGCCCCACTGC |
| NO: 4411 | platelet factor 4 variant 1 | CCAACTCATAGCCAC |
| | (PF4V1), mRNA | GCTGAAGAATGGGAG |
| | | GAAAATTTGCTTGGA |
| SEQ ID | GI 834400732 NG_042181 Homo sapiens | GGGGCTTGTGAGAGC |
| NO: 4412 | collagen type XII alpha 1 chain | TTTCTTCACTCAAGA |
| | (COL12A1), RefSeqGene on chromosome 6 | CCCTCACAGTTCACA |
| | | CTGGGGCCAGCCTTA |
| SEQ ID | GI 27805154 AY217549 Homo sapiens | GCTGGGCCAAAGGGG |
| NO: 4413 | mutL homolog 1, colon | AGGGGGAATGAAAAG |
| | cancer, nonpolyposis type 2 | AGTGTTTCTGGCAGA |
| | (E. coli) | GGAAATGGCAAGGAT |
| | (MLH1) gene, complete cds | |
| SEQ ID | GI 1063759771 NG_051548 Homo sapiens | TGTAGCTGGACCAGC |
| NO: 4414 | mitogen-activated protein | AGGAGGATTCAGGGT |
| | kinase-activated protein kinase 3 | GAGAGGTATGGACCA |
| | (MAPKAPK3), RefSeqGene on [symbol] | GAGCGTTGTGTGTGA |
| SEQ ID | GI 256985178 NG_012796 Homo sapiens | TACCCAGTGATGTCA |
| NO: 4415 | glycogen phosphorylase L | GAGCATGAGTCCAGA |
| | (PYGL), RefSeqGene on chromosome 14 | TGTGCTCTGTGCGTG |
| | | CATGGGATGGCTTCC |
| SEQ ID | GI 82880650 NM_004051 Homo sapiens | TCCTTGTGTTTGCTG |
| NO: 4416 | 3-hydroxybutyrate dehydrogenase 1 | GCTGCTTGATGAAGG |
| | (BDH1), transcript variant 1, mRNA | ACAAAGGCCATGATG |
| | | GGGTCAAGGAGCTGG |
| SEQ ID | GI 84043962 NM_015904 Homo sapiens | ACATGTCCCCACAGT |
| NO: 4417 | eukaryotic translation initiation | TCCAATGTGCCTGTT |
| | factor 5B | CACTCACCTCTCCCT |
| | (EIF5B), mRNA | TCCCCAACCCTTCTC |
| SEQ ID | GI 161377466 NM_003914 Homo sapiens | GGAGACCGGCTTTCC |
| NO: 4418 | cyclin A1 | CGCAATCATGTACCC |
| | (CCNA1), transcript variant 1, mRNA | TGGATCTTTTATTGG |
| | | GGGCTGGGGAGAAGA |
| SEQ ID | GI 666335577 NM_006494 Homo sapiens | AGGCGAGTCGGAGGA |
| NO: 4419 | ETS2 repressor factor | GGTAGAGGTGACTGA |
| | (ERF), transcript variant 1, mRNA | CATCAGTGATGAGGA |
| | | TGAGGAAGACGGGGA |
| SEQ ID | GI 14278713 AF377330 Homo sapiens | TCACGAAACAGTGGC |
| NO: 4420 | urokinase-type plasminogen activator | CACACAAATGTGAGG |
| | (PLAU) gene, complete cds | TGGGGTTGGAAGGAG |
| | | ACCCTGTCCAGTCTT |
| SEQ ID | GI 401709981 NG_032894 Homo sapiens | CTCCTCCTGGAGCTG |
| NO: 4421 | F2R like thrombin or trypsin receptor 3 | GGTGTTGACTCTGTC |
| | (F2RL3), RefSeqGene on chromosome 19 | CAAGACTCTGCTCAG |
| | | TCACTTCCTGGCTGA |
| SEQ ID | GI 31317227 NM_004430 Homo sapiens | GCCGGCGTTGGACAG |
| NO: 4422 | early growth response 3 | CAATCTCTTCCCCAT |
| | (EGR3), transcript variant 1, mRNA | GATTCCTGACTACAA |
| | | CCTCTACCACCACCC |
| SEQ ID | GI 45238846 NM_006142 Homo sapiens | GGGCGAGGAGCTCTC |
| NO: 4423 | stratifin | CTGCGAAGAGCGAAA |
| | (SFN), mRNA | CCTGCTCTCAGTAGC |
| | | CTATAAGAACGTGGT |
| SEQ ID | GI 564730481 NM_001287262 Homo sapiens | CCACTGGCCAGCATC |
| NO: 4424 | butyrophilin like 10 | TGTCAAAACTGAGGG |
| | (BTNL10), mRNA | ACAGACTCCTGTGCA |
| | | AGTCGAGGGAAAGCT |
| SEQ ID | GI 217416377 NM_138394 Homo sapiens | TCACCCGTCGTCCAT |
| NO: 4425 | heterogeneous nuclear | GTTCGAGGACTCTGT |
| | ribonucleoprotein L like | GAATCTGTGGTGGAA |
| | (HNRNPLL), transcript variant 1, mRNA | GCAGACCTCGTGGAA |
| SEQ ID | GI 577019576 NM_002569 Homo sapiens | TTCTGGACGATGGCA |
| NO: 4426 | furin, paired basic | TCGAGAAGAACCACC |
| | amino acid cleaving enzyme | CGGACTTGGCAGGCA |
| | (FURIN), transcript variant 1, mRNA | ATTATGATCCTGGGG |
| SEQ ID | GI 63477961 NM_000911 Homo sapiens | GACACGGTGACCAAG |
| NO: 4427 | opioid receptor delta 1 | ATCTGCGTGTTCCTC |
| | (OPRD1), mRNA | TTCGCCTTCGTGGTG |
| | | CCCATCCTCATCATC |
| SEQ ID | GI 20514390 AF507942 Homo sapiens | TCAAGCTGGCTGACT |
| NO: 4428 | cyclin-dependent kinase 4 | TTGGCCTGGCCAGAA |
| | (CDK4) gene, complete cds | TCTACAGCTACCAGA |
| | | TGGCACTTACACCCG |
| SEQ ID | GI 342187223 NG_029593 Homo sapiens | TTAAACCTCTCAACC |
| NO: 4429 | LYN proto-oncogene, | AAGTGGCTTTCCTGG |
| | Src family tyrosine kinase | TGCTGTCCCTGAAGC |
| | (LYN), RefSeqGene on chromosome 8 | AGGTGTGAGGCCTGA |
| SEQ ID | GI 47458816 NM_005622 Homo sapiens | AACTCCAGAGAGGGG |
| NO: 4430 | acyl-CoA synthetase | TGGGGGAACCTCAAG |
| | medium chain family member 3 | GAGTTGATGAAACAT |
| | (ACSM3), transcript variant 1, mRNA | GCCAGTGACAGCCAC |
| SEQ ID | GI 149408158 NM_005259 Homo sapiens | TCTGGTACACCAAGC |
| NO: 4431 | myostatin | AAACCCCAGAGGTTC |
| | (MSTN), mRNA | AGCAGGCCCTTGCTG |
| | | TACTCCCACAAAGAT |
| SEQ ID | GI 520975386 NM_001278651 Homo sapiens | CCTTTGCTCCACTGC |
| NO: 4432 | Ran GTPase activating protein 1 | CACATATGTGAGGAC |
| | (RANGAP1), transcript variant 1, mRNA | AGCCCAGCCAACCTT |
| | | CCTTACCTCCTACCC |
| SEQ ID | GI 1049010852 AH002976 Homo sapiens | CAGTCAGAGAACTGC |
| NO: 4433 | S protein | GTTCCCCACCCCTTC |
| | (PROS1) gene, complete cds; | CCCTTTGGAAACGTC |
| | and PS-alpha gene, complete sequence | ACACTGTGGAGGAAA |
| SEQ ID | GI 168480137 NG_007294 Homo sapiens | GAACGGAGCAGGGAG |
| NO: 4434 | hydroxyacyl-CoA dehydrogenase/ | GGGCAGTGATCTGCA |
| | 3-ketoacyl-CoAthiolase/ | AGGCTGATTAATCCC |
| | enoyl-CoA hydratase | ATACAAGACAGCGCT |
| | (trifunctiona [symbol] | |
| SEQ ID | GI 588282795 NM_002168 Homo sapiens | GCCTCAGCAATGTGA |
| NO: 4435 | isocitrate dehydrogenase | AGCTGAACGAGCACT |
| | (NADP(+)) 2, mitochondrial | TCCTGAACACCACGG |
| | (IDH2), transcript variant 1, mRNA | ACTTCCTCGACACCA |
| SEQ ID | GI 50234986 AY675319 Homo sapiens | TAGAGGGCTGGTGAC |
| NO: 4436 | transforming growth | TTGTGCTTGTCTGAA |
| | factor, beta receptor II | TGCAGGAAACCAGGC |
| | (70/80 kDa) | CTGTGTAGCCTTCGG |
| | (TGFBR2) gene, complete cds | |
| SEQ ID | GI 1039673930 NG_050913 Homo sapiens | AGTGCTAAAGCCGGG |
| NO: 4437 | nuclear receptor | CAGCAAGCTCACTGC |
| | subfamily 5 group A member 2 | ATTTGCACTTCTCCA |
| | (NR5A2), RefSeqGene on chromosome 1 | TGGGAAATGCCTCTT |
| SEQ ID | GI 225579089 NG_011672 Homo sapiens | TCTGCTGTTGACAAT |
| NO: 4438 | decorin | GGCTCTCTGGCCAAC |
| | (DCN), RefSeqGene on chromosome 12 | ACGCCTCATCTGAGG |
| | | GAGCTTCACTTGGAC |
| SEQ ID | GI 195976780 NM_005201 Homo sapiens | CTGGGAAACAGCCTG |
| NO: 4439 | C-C motif chemokine receptor 8 | GTCATCCTGGTCCTT |
| | (CCR8), mRNA | GTGGTCTGCAAGAAG |
| | | CTGAGGAGCATCACA |
| SEQ ID | GI 95104789 NM_005860 Homo sapiens | CTCCATCTGCGTTGA |
| NO: 4440 | follistatin like 3 | TGCTCAGAATCGCCT |
| | (FSTL3), mRNA | ACCTGTGCCTGCGTG |
| | | TAAACCACAGCCTCA |
| SEQ ID | GI 51537346 AY728138 Homo sapiens | GTGGTTTGTCTGACA |
| NO: 4441 | chemokine | ATGTGGGGAAACCCA |
| | (C-X-C motif) receptor 4 | GTCTGCTGGCCAGCC |
| | (CXCR4) gene, complete cds | CTTGCATGAAGTAGC |
| SEQ ID | GI 1036032480 AH002739 Homo sapiens | CCGCCCTCTGAGCAC |
| NO: 4442 | cholecystokinin | GTGTTACTGCCAGTC |
| | (CCK) gene, complete cds | TGCGTCAGCGTTGGG |
| | | TAAATACATGACTGG |
| SEQ ID | GI 37499464 AY422952 Homo sapiens | TTTCCCAAGGCGGAG |
| NO: 4443 | apolipoprotein A-1 | CCTTCAAACTGGGAC |
| | (APOA1) gene, complete cds | ACATAGTCTCTGCCG |
| | | CTGTCTTTGAGCACA |
| SEQ ID | GI 334278891 NG_029019 Homo sapiens | AGAAAGGGAACGTGG |
| NO: 4444 | CCAAT/enhancer binding protein beta | GCGAGTGCTTTGGTT |
| | (CEBPB), RefSeqGene on chromosome 20 | CAGAGAGGGGAGCCA |
| | | TTTTGCCTAAGGTCA |
| SEQ ID | GI 315360651 NG_028027 Homo sapiens | CAAGTCGTGGCTTGT |
| NO: 4445 | prodynorphin | CCACTTCCTAGCTGT |
| | (PDYN), RefSeqGene on chromosome 20 | GTGCAACCACTTCCC |
| | | AGCTCTGAGCCTCAG |
| SEQ ID | GI 167614499 NM_002286 Homo sapiens | GTGACTCCCAAATCC |
| NO: 4446 | lymphocyte activating 3 | TTTGGGTCACCTGGA |
| | (LAG3), mRNA | TCCCTGGGGAAGCTG |
| | | CTTTGTGAGGTGACT |
| SEQ ID | GI 413968625 JX877554 Homo sapiens | GGCGGGACTTGGACT |
| NO: 4447 | krueppel-like factor 1 | TGGCTGGCCTCTGAG |
| | (KLF1) gene, complete cds | AGTGAGTGCCTCCTT |
| | | AAATTTTGTGCCCTA |
| SEQ ID | GI 342349316 NG_029618 Homo sapiens | GCCTCCTGATTGGTC |
| NO: 4448 | CD28 molecule | TCTGCCTCAGTTCTT |
| | (CD28), RefSeqGene on chromosome 2 | CCCTTGCTCCAAGCC |
| | | AAGGGCAGTCAAGAT |
| SEQ ID | GI 31321969 AY114157 Homo sapiens | ATCAGGCCTGACTCG |
| NO: 4449 | inhibitor of kappaB kinase gamma | CTGCTCTTTTTGTTC |
| | (IKBKG) mRNA, complete cds | CCTTCTGTCTGCTCG |
| | | AACCACTTGCCTCGG |
| SEQ ID | GI 24286757 AY163769 Homo sapiens | GTATGCAGCTGTCAG |
| NO: 4450 | excision repair cross-complementing | CGTTGGGCTGCAAAC |
| | rodent repair deficiency, | CAGTGACATCACCGA |
| | complementation group 3 | GTACCTCAGGAAGCT |
| | (xeroc [symbol] | |
| SEQ ID | GI 336088682 NG_029094 Homo sapiens | CAGGAGCTCGGAGTG |
| NO: 4451 | apoptotic peptidase activating factor 1 | GTGGTAACAGATGCC |
| | (APAF1), RefSeqGene on chromosome 12 | CTAAGAGCAGATGCC |
| | | CTAAGGAGCACTGGA |
| SEQ ID | GI 334278891 NG_029019 Homo sapiens | AGGTTTGGGAGTCTG |
| NO: 4452 | CCAAT/enhancer binding protein beta | GTGGGAACAATGCCA |
| | (CEBPB), RefSeqGene on chromosome 20 | CCACCACTGACAGTA |
| | | GCGACACCCTTCATG |
| SEQ ID | GI 340805876 NG_029513 Homo sapiens | TGTCGGGAGTAGCAC |
| NO: 4453 | ribosomal protein 56 kinase B1 | TGCCGCTGCTGTTAC |
| | (RPS6KB1), RefSeqGene on chromosome 17 | AGCCACCAGGAGTTT |
| | | TATTTCGGGAGCAAG |
| SEQ ID | GI 291463310 NM_002169 Homo sapiens | GGTCACTCAATCTCA |
| NO: 4454 | interferon alpha 5 | ACAGCCCAGAAGCAT |
| | (IFNA5), mRNA | CTGCAACCTCCCCAA |
| | | TGGCCTTGCCCTTTG |
| SEQ ID | GI 341865606 NG_029554 Homo sapiens | TGATGAAGGTGTTGC |
| NO: 4455 | Fc fragment of IgE receptor II | TGACCTTGGAGGGGT |
| | (FCER2), RefSeqGene on chromosome 19 | TTGTTGGTGGTTGAA |
| | | GTGCTGCTGGCCATG |
| SEQ ID | GI 1174251657 KY500414 Homo sapiens | GGCGAGAGGGTGTAC |
| NO: 4456 | LTB gene, complete cds | GTCAACATCAGTCAC |
| | | CCCGATATGGTGGAC |
| | | TTCGCGAGAGGGAAG |
| SEQ ID | GI 1206876284 NG_033870 Homo sapiens | TGAGACGTCCCTCCT |
| NO: 4457 | asparagine synthetase | GCCCCTGGCTGCCTT |
| | (glutamine-hydrolyzing) | ATTTCATCTGCACCC |
| | (ASNS), RefSeqGene on chromosome 7 | AATCGTATCCATTGC |
| SEQ ID | GI 189339243 NG_007969 Homo sapiens | AACCCTGCAGGGCCA |
| NO: 4458 | cytochrome P450 family | TGCCTTGAATTCCCT |
| | 7 subfamily A member 1 | CACGGAAAGCATGAT |
| | (CYP7A1), RefSeqGene on chromosome 8 | GGAAAACCTCCAACG |
| SEQ ID | GI 8308037 AF244139 Homo sapiens | CCTGTGGGGGGCAGA |
| NO: 4459 | integrin-linked kinase 1 | GGGTTTTCACAGAGG |
| | (ILK) gene, complete cds | ACGCAGTTTGAGCTG |
| | | AATCTTGACTGCAGA |
| SEQ ID | GI 1128611453 NM_019058 Homo sapiens | CCCGACTTCGAGCTG |
| NO: 4460 | DNA damage inducible transcript 4 | CTCAGTGACCCTGAG |
| | (DDIT4), mRNA | GATGAACACTTGTGT |
| | | GCCAACCTGATGCAG |
| SEQ ID | GI 769468216 NM_003246 Homo sapiens | AATCCCTGCTTTGCC |
| NO: 4461 | thrombospondin 1 | GGCGTGAAGTGTACT |
| | (THBS1), mRNA | AGCTACCCTGATGGC |
| | | AGCTGGAAATGTGGT |
| SEQ ID | GI 440502998 JX512445 Homo sapiens | AGGTCTTTGCACGGT |
| NO: 4462 | integrin, alpha V | TTGGCAGTGCCATAG |
| | (ITGAV) gene, complete cds | CTCCTTTGGGAGATC |
| | | TGGACCAGGATGGTT |
| SEQ ID | GI 1036030212 AH006319 Homo sapiens | AGTTGGTCTCTTCCC |
| NO: 4463 | succinate dehydrogenase subunit C | ATGGCGATGTCCATC |
| | (SDHC) gene, complete cds | TGCCACCGTGGCACT |
| | | GGTATTGCTTTGAGT |
| SEQ ID | GI 806904734 NG_008957 Homo sapiens | AACTGCAACCACAGT |
| NO: 4464 | monoamine oxidase A | TATCCCCACCCAAGC |
| | (MAOA), RefSeqGene on chromosome X | CGTTGTGGTCTTTTG |
| | | CCTTTGTCCAAGCCA |
| SEQ ID | GI 292658763 NM_014143 Homo sapiens | ACACAGCCCTCCTAA |
| NO: 4465 | CD274 molecule | GAGGCTTCCTGGAGG |
| | (CD274), transcript variant 1, mRNA | TTTCGAGATTCAGAT |
| | | GCCCTGGGAGATCCC |
| SEQ ID | GI 70995318 NM_002506 Homo sapiens | GAGCAAGCGGTCATC |
| NO: 4466 | nerve growth factor | ATCCCATCCCATCTT |
| | (NGF), mRNA | CCACAGGGGCGAATT |
| | | CTCGGTGTGTGACAG |
| SEQ ID | GI 166706778 EU332848 Homo sapiens | GCTTTGGCAAGCAGG |
| NO: 4467 | integrin, beta 5 | GGTGCTACTGAGTGT |
| | (ITGB5) gene, complete cds | GTGAGTATTTCCCTC |
| | | TGGACGTGTGTCCCC |
| SEQ ID | GI 343790959 NG_029764 Homo sapiens | GGCAGCTTTAGGTAT |
| NO: 4468 | AKT serine/threonine kinase 3 | GCTGTGGTTGGAAGG |
| | (AKT3), RefSeqGene on chromosome 1 | CTGTTGGCATGGGAA |
| | | AGCTGGAAGTGTGGG |
| SEQ ID | GI 1049480218 NG_051308 Homo sapiens | CTGGTGATAAGAATG |
| NO: 4469 | ras homolog family member A | GGGTAGCAGAGCTGG |
| | (RHOA), RefSeqGene on chromosome 3 | GTGCAGTGCTGGGCA |
| | | GTGTTGAATGGGACT |
| SEQ ID | GI 385275063 NG_032144 Homo sapiens | ATACAGGAGGCAGAG |
| NO: 4470 | transient receptor potential cation | ACACAGCTGGACTAG |
| | channel subfamily V member 3 | AACCCAGGCTTTAGG |
| | (TRPV3), RefSeqGene on [symbol] | TTCCCAGGCAAGGGT |
| SEQ ID | GI 527317415 NM_000304 Homo sapiens | AACTGGAGCCCTTGC |
| NO: 4471 | peripheral myelin protein 22 | AAAAACACGGCTTGT |
| | (PMP22), transcript variant 1, mRNA | GGCATTGGCATACTT |
| | | GCCCTTACAGGTGGA |
| SEQ ID | GI 41353192 AY486465 Homo sapiens | AGAAATGCAGAACAC |
| NO: 4472 | glutathione transferase kappa 1 | GGCCACCTCTTAGCC |
| | (GSTK1) gene, complete cds | CAACGATGGCAGTTT |
| | | TGGGGAAAACTGGCC |
| SEQ ID | GI 340805836 NG_029500 Homo sapiens | CTCATCATGGAGCCC |
| NO: 4473 | fatty acid binding protein 6 | ATGTGGTGTGGGCAG |
| | (FABP6), RefSeqGene on chromosome 5 | TGACCAGAGTTCAGG |
| | | GTCATCATGCACACA |
| SEQ ID | GI 334358873 NG_029029 Homo sapiens | CTGGTTTGCAGGCCT |
| NO: 4474 | sterol regulatory element | CTTCAATAAAGGTGG |
| | binding transcription factor 1 | GGCTGTGCTAGGCAT |
| | (SREBF1), RefSeqGene on chromoso [symbol] | TGGGGATGCAGCAAT |
| SEQ ID | GI 584277093 NM_001675 Homo sapiens | GGAAGAATCCCCTGG |
| NO: 4475 | activating transcription factor 4 | TCTCCGTGAGCGTCC |
| | (ATF4), transcript variant 1, mRNA | ATTTTGTGGAACCTG |
| | | AGTTGCAAGCAGGGA |
| SEQ ID | GI 374252456 NM_001252406 Homo sapiens | CAGGGCTGTGCTAGC |
| NO: 4476 | zinc finger and BTB domain containing 7B | TGCCTGTAGCCACTA |
| | (ZBTB7B), transcript variant 3, mRNA | CTTCAAGAAGCTTTT |
| | | CACTGAGGGCGGTGG |
| SEQ ID | GI 269914110 NG_015981 Homo sapiens | TTCCCTCCAATTCCC |
| NO: 4477 | anti-Mullerian hormone receptor type 2 | ACTTGCCCTCCACCT |
| | (AMHR2), RefSeqGene on chromosome 12 | GCTACTCTGTTATCT |
| | | ACTGGCTCTGCCCGT |
| SEQ ID | GI 662236863 NG_034192 Homo sapiens | TCCCTGTGCACAAGT |
| NO: 4478 | diacylglycerol O-acyltransferase 1 | GGTGCATCAGGTAGG |
| | (DGAT1), RefSeqGene on chromosome 8 | TGGGGTGTGTGTGTG |
| | | TGTGATGTGGAACAT |
| SEQ ID | GI 15545992 AY029254 Homo sapiens | CACCGGCTTCATGTG |
| NO: 4479 | Be 1-2 modifying factor | CAGCAACACCAGCAG |
| | (BMF) mRNA, complete cds | AACCAAAATCGTGTG |
| | | TGGTGGCAGATCCTC |
| SEQ ID | GI 187171274 NM_030662 Homo sapiens | GAAGAAGCGGCTGGA |
| NO: 4480 | mitogen-activated | AGCCTTTCTCACCCA |
| | protein kinase kinase 2 | GAAAGCCAAGGTCGG |
| | (MAP2K2), mRNA | CGAACTCAAAGACGA |
| SEQ ID | GI 89142743 NM_023068 Homo sapiens | TGCAAGATGTGCCTG |
| NO: 4481 | sialic acid binding ig like lectin 1 | CAGGTGATGACACCT |
| | (SIGLEC1), mRNA | ATGTTTGCACAGCCC |
| | | AAAACTTGCTGGGCT |
| SEQ ID | GI 1168024008 KY497359 Homo sapiens | TCCTCACGCCTCCCC |
| NO: 4482 | HLA-E | TATGTGTCTTAGGGG |
| | (HLA-E) gene, complete cds | ACTCTGGCTTCTCTT |
| | | TTTGCAAGGGCCTCT |
| SEQ ID | GI 224967082 NG_011581 Homo sapiens | GTGCCTGGACAATGC |
| NO: 4483 | thyroid peroxidase | TCTGATCCACCCAGG |
| | (TPO), RefSeqGene on chromosome 2 | TGTTGGCAGCATTGT |
| | | TGCAAAGGTCACCTC |
| SEQ ID | GI 1025608649 NG_047040 Homo sapiens | GCAGTGTCCCCCATC |
| NO: 4484 | calcium/calmodulin dependent | CCGACCCCACAGGAA |
| | protein kinase 11 alpha | GACAAATTAGAAAAA |
| | (CAMK2A), RefSeqGene on chromos [symbol] | GCCCCCTCTGGATCC |
| SEQ ID | GI 313747578 NM_015162 Homo sapiens | GCCAAGGTTCGCCAG |
| NO: 4485 | acyl-CoA synthetase | GCACTGGGATTTGCC |
| | bubblegum family member 1 | AAGTGTCAAAAGAAC |
| | (ACSBG1), transcript variant 1, mRNA | TTCTATGGAGCGGCC |
| SEQ ID | GI 297374833 NM_006184 Homo sapiens | AATGAGGGCATGGGG |
| NO: 4486 | nucleobindin 1 | TGGTCCCTCAAGACC |
| | (NUCB1), mRNA | TTCCCCTACCTTTTG |
| | | TGGAACCAGTGATGC |
| SEQ ID | GI 21105786 AF512504 Homo sapiens | TTCCACGGGAAAGTG |
| NO: 4487 | X-ray repair complementing | TTGTAGGGGAAACTC |
| | defective repair in Chinese | CAGGCAGAGTGTCGG |
| | hamster cells 1 | GGGATTAACGGGAGA |
| | (XRCC1) gene, complete [symbol] | |
| SEQ ID | GI 398303864 NG_032876 Homo sapiens | TCCTCACAAGGACTC |
| NO: 4488 | major histocompatibility | ATGGCTTGCCCCATA |
| | complex, class II, DQ alpha 1 | GCATCACATCTGTGC |
| | (HLA-DQA1), RefSeqGene on chromos [symbol] | TGCTTACCAGCTGCG |
| SEQ ID | GI 22027540 NM_005707 Homo sapiens | TGGCATTCCCCTACC |
| NO: 4489 | programmed cell death 7 | CTGAAGTTATGCTTT |
| | (PDCD7), mRNA | CCTTCTGTGCTCTGT |
| | | GCTGGCCAGAGGTGC |
| SEQ ID | GI 994323650 NG_011530 Homo sapiens | AGCTGGGATGCAAAC |
| NO: 4490 | 2′-5′-oligoadenylate synthetase 1 | CCTAAACTGCCAGCC |
| | (OAS1), RefSeqGene on chromosome 12 | CTCAAAGCCTGTGCT |
| | | CTTAATCTCCACCCT |
| SEQ ID | GI 1049480218 NG_051308 Homo sapiens | ATGGGTGGACTGGTG |
| NO: 4491 | ras homolog family member A | GGGATTCTGGCGTAA |
| | (RHOA), RefSeqGene on chromosome 3 | AATGGTATGGTGGCA |
| | | TGAGTCAGGGCACAA |
| SEQ ID | GI 425905339 NM_080725 Homo sapiens | GAGTCCCTGGGGAAA |
| NO: 4492 | sulfiredoxin 1 | GGGAACAGAGTAAGG |
| | (SRXN1), mRNA | CAGGCCTTGTTCTCA |
| | | CTGCCCTCTAAGGGA |
| SEQ ID | GI 225543278 NG_011648 Homo sapiens | GACCAGGGAACTAGG |
| NO: 4493 | angiotensin 1 converting enzyme | TGGTACATGCTTGCA |
| | (ACE), RefSeqGene on chromosome 17 | CCCAGCATTCAAAGT |
| | | GAGAGGCCTGTGCCA |
| SEQ ID | GI 1024846763 NG_046978 Homo sapiens | CTCCACCCACAGACA |
| NO: 4494 | Charcot-Leyden crystal galectin | CAAGGTTAGGACACC |
| | (CLC), RefSeqGene on chromosome 19 | TCCATTTCCACTGCC |
| | | TCCCCTCACCCAGTA |
| SEQ ID | GI 260593656 NG_013016 Homo sapiens | GGTCCTAGACTCAAC |
| NO: 4495 | phosphoglycerate mutase 2 | TCCGTGCCACCCTTC |
| | (PGAM2), RefSeqGene on chromosome 7 | AGCCTACGTTGTGGG |
| | | TTCCTGCTAAGCTGA |
| SEQ ID | GI 571330780 NM_001136022 Homo sapiens | CACTTGCCATTGCCT |
| NO: 4496 | nuclear factor of activated T-cells 4 | CAGCTTTCCCCTAAA |
| | (NFATC4), transcript variant 1, mRNA | CACGGTGTCTGTGGT |
| | | CAGGGTTGGTGAGGA |
| SEQ ID | GI 77744394 DQ230990 Homo sapiens | AGCTGCCAAGTAGGT |
| NO: 4497 | peroxiredoxin 6 | GGCCGAAAGACTTTT |
| | (PRDX6) gene, complete cds | TGTCCAGAGCCGCCT |
| | | CCAATGGCCAAGATA |
| SEQ ID | GI 1147489694 NG_016780 Homo sapiens | ACACCTGTTCCTCAG |
| NO: 4498 | acyl-CoA thioesterase 9 | GCCTCCTCCCTCAAT |
| | (ACOT9), RefSeqGene on chromosome X | ACCGTCTCTTTGACT |
| | | TGCTCTCCTGGCACC |
| SEQ ID | GI 182995 M68891 Human | CCAGACGAGGTGGAC |
| NO: 4499 | GATA-binding protein | GTCTTCTTCAATCAC |
| | (GATA2) mRNA, complete cds | CTCGACTCGCAGGGC |
| | | AACCCCTACTATGCC |
| SEQ ID | GI 340805891 NG_029487 Homo sapiens | AGCCTCCTTGGATAA |
| NO: 4500 | BCL2 related protein A1 | GCTAAGCACATTCCA |
| | (BCL2A1), RefSeqGene on chromosome 15 | CCCTCAAGGCCTTTG |
| | | CACTTGCTGCTCCCT |
| SEQ ID | GI 1036030581 AH008227 Homo sapiens | AGCAGCAGGAACTTG |
| NO: 4501 | chromosome 1 PEA15 protein | GGCCTCTTCTAGGAA |
| | (PEA15) gene, complete cds | TGGTGTAGACCCAAG |
| | | AAAGGAGGGGCTCGT |
| SEQ ID | GI 1028908930 NG_050628 Homo sapiens | TGCAGCAGTTCTAGC |
| NO: 4502 | nuclear factor kappa B subunit 1 | CAAGTGCATGCATAC |
| | (NFKB1), RefSeqGene on chromosome 4 | AGGCCTTGAGGTGGA |
| | | GCTGCACAGTTGACT |
| SEQ ID | GI 15545992 AY029254 Homo sapiens | GGAGCCATCTCAGTG |
| NO: 4503 | Be 1-2 modifying factor | TGTGGAGGAGCTGGA |
| | (BMF) mRNA, complete cds | GGATGATGTGTTCCA |
| | | ACCAGAGGATGGGGA |
| SEQ ID | GI 61744461 NM_003405 Homo sapiens | TGCACCTGAGCAAGC |
| NO: 4504 | tyrosine 3-monooxygenase/ | CTGCCTCTTAGCCAA |
| | tryptophan 5-monooxygenase | ACAAGCCTTCGATGA |
| | activation protein eta | TGCCATAGCTGAGCT |
| | (YWHAH), [symbol] | |
| SEQ ID | GI 182739 L10820 Human | AGAGGCATCATCCGG |
| NO: 4505 | N-formyl peptide receptor | TTCATCATTGGCTTC |
| | (FPR1) gene, complete cds | AGCGCACCCATGTCC |
| | and Alu repeats | ATCGTTGCTGTCAGT |
| SEQ ID | GI 205277450 NM_001135054 Homo sapiens | GTGCCCGCACAGACT |
| NO: 4506 | single Ig and TIR domain containing | TCTACTGCCTGGTGT |
| | (SIGIRR), transcript variant 1, mRNA | CCAAGGATGATATGT |
| | | AGCTCCCACCCCAGA |
| SEQ ID | GI 874507491 NG_042218 Homo sapiens | GGATTGCCTTCCTGC |
| NO: 4507 | surfactant protein D | CTTCCCAGGATGTGG |
| | (SFTPD), RefSeqGene on chromosome 10 | AGCTGGTATACTCCT |
| | | TCCCACTCTGGTGGA |
| SEQ ID | GI 254039585 NG_012387 Homo sapiens | TCACACCTCACTTCT |
| NO: 4508 | aldolase, fructose-bisphosphate B | GCTTTGTCCTAGGCA |
| | (ALDOB), RefSeqGene on chromosome 9 | AACTCCAAGCACACT |
| | | GGCAAGGACAGGGCC |
| SEQ ID | GI 1024336774 NG_046918 Homo sapiens | AGTTGGAGGGAGTGT |
| NO: 4509 | DExD/H-box helicase 58 | TGTCACAAGCCAAGG |
| | (DDX58), RefSeqGene on chromosome 9 | AATGCCAGCAGCTAC |
| | | CAGAAACCGGAAGGG |
| SEQ ID | GI 430727969 NG_033033 Homo sapiens | TTCAGATGCTGACAA |
| NO: 4510 | ISG15 ubiquitin-like modifier | TTTTGGTGACCCCTG |
| | (ISG15), RefSeqGene on chromosome 1 | AGATTTGCTGGGAGA |
| | | CGGGGAAGGGCCAGG |
| SEQ ID | GI 325651826 NM_001443 Homo sapiens | TCACCGCTGGGTCCA |
| NO: 4511 | fatty acid binding protein 1 | AAGTGATCCAAAACG |
| | (FABP1), mRNA | AATTCACGGTGGGGG |
| | | AGGAATGTGAGCTGG |
| SEQ ID | GI 2789671 AF040714 Homo sapiens | AGCAGCCGCCAGCGC |
| NO: 4512 | homeobox protein A10 | CAATAAATAAGCCCA |
| | (HOXA10) gene, complete cds | TTAACCGGCGAAGTT |
| | | CGAGTGTACGATCCC |
| SEQ ID | GI 297307113 NG_021395 Homo sapiens | GCAAGGGAAGAAAAA |
| NO: 4513 | erythropoietin receptor | GGGCAGCAATGACCC |
| | (EPOR), RefSeqGene on chromosome 19 | TCAATGGGCCATTTG |
| | | AGGGATCTGGCCTGG |
| SEQ ID | GI 237858689 NG_012090 Homo sapiens | CTGCCACAGGAGGTT |
| NO: 4514 | interleukin 13 | CCCCAAGTGAAAACA |
| | (IL13), RefSeqGene on chromosome 5 | GTCAGCCACCTGCCC |
| | | TATAGGAAACAGCCC |
| SEQ ID | GI 209364548 NM_001912 Homo sapiens | CAGCTGGGGTGAAGA |
| NO: 4515 | cathepsin L | ATGGGGCATGGGTGG |
| | (CTSL), transcript variant 1, mRNA | CTACGTAAAGATGGC |
| | | CAAAGACCGGAGAAA |
| SEQ ID | GI 37577125 NM_194315 Homo sapiens | GGCCTTGGGTACCTG |
| NO: 4516 | ubiquitin conjugating enzyme E2 J2 | CAGAGCCTTTTCTCT |
| | (UBE2J2), transcript variant 1, mRNA | GAATTCCTTATGTCC |
| | | GGTGGGCCAGAAGCC |
| SEQ ID | GI 45505189 AY572796 Homo sapiens | TCAGTCCTGCCGAGG |
| NO: 4517 | signal transducer and | CAGATGGCTTCCTCC |
| | activator of transcription 3 | AGCTCTGCTTACTGA |
| | (acute-phase response factor) | ATGCGAAGTCACAGT |
| | (STAT3) gene, c [symbol] | |
| SEQ ID | GI 343098515 NG_029659 Homo sapiens | CCCCATTGCACGCAG |
| NO: 4518 | bone morphogenetic protein 1 | GACATAGCAGAGGCT |
| | (BMP1), RefSeqGene on chromosome 8 | CAGAGGTTGCGTGGT |
| | | TTGCTTGATGACTCA |
| SEQ ID | GI 698320876 NM_015995 Homo sapiens | AGAGCCCTGGGTGCT |
| NO: 4519 | Kruppel like factor 13 | TGTGGTGCAAAGATC |
| | (KLF13), transcript variant 1, mRNA | TTCATGCCCAGTTCC |
| | | AAAGTGAGGTCCCTC |
| SEQ ID | GI 117606583 EF064766 Homo sapiens | TGCTGAAGACTGCTG |
| NO: 4520 | chemokine | CCTGTCTGTGACCCA |
| | (C-C motif) ligand 19 | GAAACCCATCCCTGG |
| | (CCL19) gene, complete cds | GTACATCGTGAGGAA |
| SEQ ID | GI 170763484 NG_007481 Homo sapiens | ACTGAGGCAGGAGAT |
| NO: 4521 | complement C3b/C4b receptor 1 | GGGCAGGGCTTGTGT |
| | (Knops blood group) | TCTGGTCACAATCCT |
| | (CR1), RefSeqGene on | GCTGACCAAAACAGG |
| | chromosome 1 [symbol] | |
| SEQ ID | GI 695917236 NM_021010 Homo sapiens | AGGCCCAGGCTGAGT |
| NO: 4522 | defensin alpha 5 | CACTCCAGGAAAGAG |
| | (DEFA5), mRNA | CTGATGAGGCTACAA |
| | | CCCAGAAGCAGTCTG |
| SEQ ID | GI 1060856491 NM_005526 Homo sapiens | TCAAACCGGATCCTG |
| NO: 4523 | heat shock transcription factor 1 | GGGGTGAAGAGAAAG |
| | (HSF1), mRNA | ATCCCCCTGATGCTG |
| | | AACGACAGTGGCTCA |
| SEQ ID | GI 263191400 NG_015828 Homo sapiens | TCTTCCCAGGTCTTG |
| NO: 4524 | glutamic-pyruvic transaminase | GCCCTCTGTGTTAAC |
| | (GPT), RefSeqGene on chromosome 8 | CCTGATCTTCTGAGC |
| | | AGCCCCAACTTCCCT |
| SEQ ID | GI 576795860 NM_001549 Homo sapiens | AGCCTGGTCACCAGC |
| NO: 4525 | interferon induced protein | TTTTCGGAACAGCAG |
| | with tetratricopeptide repeats 3 | AGACACAGAGGGCAG |
| | (1 FITS), transcript | TCATGAGTGAGGTCA |
| | variant 1, mR [symbol] | |
| SEQ ID | GI 343098515 NG_029659 Homo sapiens | AGTGTGTACCGTGTG |
| NO: 4526 | bone morphogenetic protein 1 | CACATGTATGTGTGT |
| | (BMP1), RefSeqGene on chromosome 8 | GCCTGTGGACCAGCA |
| | | CCTGTGTTGCCACAT |
| SEQ ID | GI 187171274 NM_030662 Homo sapiens | CCTGGACCAGGTGCT |
| NO: 4527 | mitogen-activated | GAAAGAGGCCAAGAG |
| | protein kinase kinase 2 | GATTCCCGAGGAGAT |
| | (MAP2K2), mRNA | CCTGGGGAAAGTCAG |
| SEQ ID | GI 19909347 AF495471 Homo sapiens | GTGTGCTGGGGAAGC |
| NO: 4528 | thrombomodulin | TCCATGCACAGTGCC |
| | (THBD) gene, complete cds | CTATGGAGATAACAA |
| | | GCTGAGCCATGCTCC |
| SEQ ID | GI 296923725 NG_012838 Homo sapiens | AGGCTTCATGGGGGT |
| NO: 4529 | glucose-6-phosphate isomerase | TGGAGGGTCTGCTTT |
| | (GPI), RefSeqGene on chromosome 19 | GAAGATGGCTCACTA |
| | | GCAGTGCTGCCTTTG |
| SEQ ID | GI 390979633 NM_004135 Homo sapiens | CCTGCCACCGTCGCA |
| NO: 4530 | isocitrate dehydrogenase 3 | CAAATCTCGAAACAA |
| | (NAD(+)) gamma | CATCCTTCGCACCAG |
| | (IDH3G), transcript variant 1, mRNA | CCTGGACCTCTATGC |
| SEQ ID | GI 300863108 NG_023389 Homo sapiens | GAGCGTCCATGACCA |
| NO: 4531 | acetyl-CoA acetyltransferase 2 | CTGTGCACACACAGC |
| | (ACAT2), RefSeqGene on chromosome 6 | AGGTTTCATGAGAAG |
| | | CTGGAGCACTGGCTC |
| SEQ ID | GI 350276269 NG_030323 Homo sapiens | TGATCACCATCTTAC |
| NO: 4532 | sialic acid binding Ig like lectin 11 | TGAGCACATTGCCAC |
| | (SIGLEC11), RefSeqGene on chromosome 19 | TGCAGCCAGCAACTG |
| | | AGAAGCCAGCGCACA |
| SEQ ID | GI 8133099 AF266756 Homo sapiens | GGAGTCACGTGCAGC |
| NO: 4533 | sphingosine kinase | CCCTTTTGGCTGAGG |
| | (SPHK1) mRNA, complete cds | CTGAAATCTCCTTCA |
| | | CGCTGATGCTCACTG |
| SEQ ID | GI 356460958 NG_030484 Homo sapiens | CAGTGCGTCTGCATG |
| NO: 4534 | adenosine kinase | AATTCCTGGCAGCTC |
| | (ADK), RefSeqGene on chromosome 10 | TCCAAAATGGGCTCA |
| | | GGGCTTGTGAGGACT |
| SEQ ID | GI 197276631 NM_003670 Homo sapiens | AACCAAGCCTTGGCA |
| NO: 4535 | basic helix-loop-helix family member e40 | CCGCCAGATCCTTTC |
| | (BHLHE40), mRNA | TGTAGGCTAATTCCT |
| | | CTTGCCCAACGGCAT |
| SEQ ID | GI 83367074 NM_175617 Homo sapiens | AGAGCAGCCAGTTGC |
| NO: 4536 | metallothionein 1E | AGGGCTCCATTCTGC |
| | (MT1E), mRNA | TTTCCAACTGCCTGA |
| | | CTGCTTGTTCGTCTC |
| SEQ ID | GI 686223163 NG_034261 Homo sapiens | TGAGCCACTTGCTTG |
| NO: 4537 | primase | GGCCGCTGCCATTCT |
| | (DNA) subunit 2 | GTGGAGTGTGCTTTC |
| | (PRIM2), RefSeqGene on chromosome 6 | ACTACTATTGCCTCC |
| SEQ ID | GI 423091980 JX512648 Homo sapiens | CTGAGGGACAGCGAG |
| NO: 4538 | SH2 domain containing 1A | AGCGTGCCAGGCGTG |
| | (SH2D1A) mRNA, complete cds | TACTGCCTATGTGTG |
| | | CTGTATCACGGTTAC |
| SEQ ID | GI 237874189 NG_012169 Homo sapiens | ATTGCATCTGTTTCC |
| NO: 4539 | angiopoietin like 4 | TGCCCCCACCTCTTC |
| | (ANGPTL4), RefSeqGene on chromosome 19 | CTTACATGCCGTGTG |
| | | TGTGATTGGGCCACT |
| SEQ ID | GI 238018073 NG_012199 Homo sapiens | ACATGTTGGGGGAGT |
| NO: 4540 | ketohexokinase | CGTGTTGTAATGGGG |
| | (KHK), RefSeqGene on chromosome 2 | GCAACGCTAGGCTGC |
| | | TGTTGGTGAATGACG |
| SEQ ID | GI 197304767 NM_014674 Homo sapiens | GGGCAGCTGATGAAC |
| NO: 4541 | ER degradation enhancing | ACCTGGATTGACTCT |
| | alpha-mannosidase like protein 1 | CTGCAGGCCTTTTTC |
| | (EDEM1), mRNA | CCTGGACTGCAGGTG |
| SEQ ID | GI 1024336777 NG_046929 Homo sapiens | ACCCTCACTTCCATC |
| NO: 4542 | BTG anti-proliferation factor 2 | ACCCCATTCCCCCTC |
| | (BTG2), RefSeqGene on chromosome 1 | TTCAGTGGGTGAAGT |
| | | CACTGGTCAGCACAA |
| SEQ ID | GI 263191773 NG_015830 Homo sapiens | ACCCTGCGCACTGGA |
| NO: 4543 | insulin receptor substrate 1 | AGATCTGGCCAGCAA |
| | (IRS1), RefSeqGene on chromosome 2 | CCTTGTTATTTTCAC |
| | | CAGCCCTCTCCCATG |
| SEQ ID | GI 325910849 NG_028199 Homo sapiens | AATGGTGGGGCTTTG |
| NO: 4544 | scavenger receptor class B member 1 | GGGAGCAGATGGAGG |
| | (SCARB1), RefSeqGene on chromosome 12 | AACTGGGGGTAAAAC |
| | | AGCGTGTTCCAGTTT |
| SEQ ID | GI 42716353 AY549314 Homo sapiens | GGGCACTAATCCAGC |
| NO: 4545 | tyrosine kinase 2 | CCTTAAATCCCCCAG |
| | (TYK2) gene, complete cds | CTTCCAAACTTGAGG |
| | | CCCACCATCTCCACC |
| SEQ ID | GI 117422442 NM_001242 Homo sapiens | GGCCCTTTCCTGTGT |
| NO: 4546 | CD27 molecule | ACACGTGACAGAGTG |
| | (CD27), mRNA | CCTTTTCGAGACTGG |
| | | CAGGGACGAGGACAA |
| SEQ ID | GI 260166681 NM_138387 Homo sapiens | CTGGCCCTCATGCTA |
| NO: 4547 | glucose-6-phosphatase | GGCACCAGCCTCATC |
| | catalytic subunit 3 | TATTGGACCCTCTTT |
| | (G6PC3), transcript | ACACTGGGCCTGGAT |
| | variant 1, mRNA | |
| SEQ ID | GI 156616272 NM_002664 Homo sapiens | TCGCTGCTCAATGAG |
| NO: 4548 | pleckstrin | GGGTATCTGCAGCCT |
| | (PLEK), mRNA | GCTGGAGACATGTCC |
| | | AAGAGTGCAGTGGAT |
| SEQ ID | GI 7407497 AF243044 Homo sapiens | ACGCAAAAGCTACCC |
| NO: 4549 | ECSIT | CATGCTCAAGTTGGT |
| | (ECSIT) mRNA, complete cds | GCGCCTGAAGCTGTG |
| | | GTTCCCTCGATTCAT |
| SEQ ID | GI 260763977 NG_013058 Homo sapiens | TCCCAGCCTCTGTTG |
| NO: 4550 | 5-hydroxytryptamine receptor 3A | AAAGGAACTCATGAG |
| | (HTR3A), RefSeqGene on chromosome 11 | CCAGCCCCTTCACTC |
| | | TCAGCCAGCTCTGTT |
| SEQ ID | GI 197116369 NG_008383 Homo sapiens | ACCCAGTGCTGGAGC |
| NO: 4551 | cytochrome P450 family 2 | CCCAGCTTCTAACAG |
| | subfamily E member 1 | GGCACTATTTCCCTC |
| | (CYP2E1), RefSeqGene on | CTGTAGGCATCACTG |
| | chromosome 10 [symbol] | |
| SEQ ID | GI 336455046 NG_029166 Homo sapiens | AGTTGGTGCTCCTGG |
| NO: 4552 | SRY-box 4 | AGCTGCTTTCTCCTT |
| | (SOX4), RefSeqGene on chromosome 6 | GATTTGCCCATGCTT |
| | | ACCTCCAGCAGTGGC |
| SEQ ID | GI 189409110 NM_153000 Homo sapiens | ACAACCTCGACCACC |
| NO: 4553 | APC down-regulated 1 | TGGTCGAGGAGCTCT |
| | (APCDD1), mRNA | TCCTTGGTGACATTC |
| | | ACACTGATGCCACCC |
| SEQ ID | GI 425905339 NM_080725 Homo sapiens | CCCTTGGCCCGAGCC |
| NO: 4554 | sulfiredoxin 1 | AGCTACCATTGCACC |
| | (SRXN1), mRNA | TCTAGCTGCAAACAA |
| | | AGCTAAGACAGCAGG |
| SEQ ID | GI 40288420 AY505111 Homo sapiens | GGAGGCAGCGGCTTT |
| NO: 4555 | arachidonate 15-lipoxygenase | TATGCTCAAAAGGAA |
| | (ALOX15) gene, complete cds | TGAGTCCCAGTGGCT |
| | | GGCACAGAGAAGGCA |
| SEQ ID | GI 270048011 GU143397 Homo sapiens | GCGAAGATGGCAAGG |
| NO: 4556 | collagen, type III, alpha 1 | ATGGATCACCTGGAG |
| | (COL3A1) gene, complete cds | AACCTGGTGCAAATG |
| | | GGCTTCCAGGAGCTG |
| SEQ ID | GI 14423572 AF388025 Homo sapiens | TGCAGGGCCAACATC |
| NO: 4557 | colony stimulating factor 3 | CTCTGGAAGGGACAT |
| | (granulocyte) | GGGAGAATATTAGGA |
| | (CSF3) gene, complete cds | GCAGTGGAGCTGGGG |
| SEQ ID | GI 41872630 NM_004104 Homo sapiens | ACGGTCGCTTCCTGG |
| NO: 4558 | fatty acid synthase | AAATTGGCAAATTCG |
| | (FASN), mRNA | ACCTTTCTCAGAACC |
| | | ACCCGCTCGGCATGG |
| SEQ ID | GI 338827642 NG_029226 Homo sapiens | ACCACAATGCCCAGC |
| NO: 4559 | nuclear factor of activated T-cells 1 | ATGTGGGGAAGAGAG |
| | (NFATC1), RefSeqGene on chromosome 18 | AGGAAAGGTGGGGTT |
| | | TATCCTCACCCGAGT |
| SEQ ID | GI 145312264 NM_033266 Homo sapiens | ACGTGCTTTCTGGTG |
| NO: 4560 | endoplasmic reticulum | GCAGCCACCCCTTTG |
| | to nucleus signaling 2 | GAGACAGTCTTTATC |
| | (ERN2), transcript variant 1, mRNA | GCCAGGCAAACATCC |
| SEQ ID | GI 224494751 NM_138801 Homo sapiens | TGGCTCCAGGCCATG |
| NO: 4561 | galactose mutarotase | TCTAATGACCAGCTC |
| | (GALM), mRNA | GATTCCCTGTGCAGT |
| | | TCAGAGGGCAAGTGA |
| SEQ ID | GI 342187247 NG_029422 Homo sapiens | AGCAGCTGTGGTCAG |
| NO: 4562 | major histocompatibility | GCTGCTGACCTTTCT |
| | complex, class 1, C | CTCAGGCCTTGTTCT |
| | (HLA-C), RefSeqGene on chromosome 6 | CTGCCTCACACTCAA |
| SEQ ID | GI 388490278 NG_032761 Homo sapiens | ACCATCCCTATCTAG |
| NO: 4563 | TNF alpha induced protein 3 | CAAGCCATGCCTGCT |
| | (TNFAIP3), RefSeqGene on chromosome 6 | TCCTGGTTTGTGCAT |
| | | GTTGCCCTGTGTGCT |
| SEQ ID | GI 156105692 NM_001037335 Homo sapiens | TGGAGGCTGCCCTTT |
| NO: 4564 | helicase with zinc finger 2 | GACCTTTGGACGCCA |
| | (HELZ2), transcript variant 1, mRNA | CCTGTAATCACTCTG |
| | | GATGGTGTCTGTGGA |
| SEQ ID | GI 567316248 NM_022128 Homo sapiens | AACCAGTGTGTCCAA |
| NO: 4565 | ribokinase | GCTGCTCGGCTTGGA |
| | (RBKS), transcript variant 1, mRNA | GCAATGACGTCCATG |
| | | GTGTGTAAGGTTGGC |
| SEQ ID | GI 527317409 NM_015077 Homo sapiens | TGTCATGGGTGCCCG |
| NO: 4566 | sterile alpha and TIR motif | CAACTTTGTGTTGGT |
| | containing 1 (SARM1), mRNA | GCTATCACCTGGAGC |
| | | ACTGGACAAGTGCAT |
| SEQ ID | GI 45238846 NM_006142 Homo sapiens | GAGGCCATGGCTGAT |
| NO: 4567 | stratifin | CTGCACACCCTCAGC |
| | (SFN), mRNA | GAGGACTCCTACAAA |
| | | GACAGCACCCTCATC |
| SEQ ID | GI 1049010976 AH002711 Homo sapiens | TGCTTGCCCATGTTT |
| NO: 4568 | platelet glycoprotein IIIa | GGCTACAAACACGTG |
| | (ITGB3) gene, complete cds | CTGACGCTAACTGAC |
| | | CAGGTGACCCGCTTC |
| SEQ ID | GI 182739 L10820 Human N-formyl | CCCATCCAATGGATG |
| NO: 4569 | peptide receptor | GAGAAGCACCTCCCA |
| | (FPR1) gene, complete cds | TCCATCTGGCCAAGG |
| | and Alu repeats | TAACCGCAGAGACGA |
| SEQ ID | GI 324021728 NG_028155 Homo sapiens | AGAGGGTCTTCAGCC |
| NO: 4570 | indoleamine 2,3-dioxygenase 1 | CTGCGTGTGCAGATG |
| | (IDO1), RefSeqGene on chromosome 8 | ACAAGACAGAGGCTG |
| | | GTGTTTCCAGACAGG |
| SEQ ID | GI 237858689 NG_012090 Homo sapiens | ACCAGGCCTGGTGCA |
| NO: 4571 | interleukin 13 | CGTGCTTTATGTGTC |
| | (IL13), RefSeqGene on chromosome 5 | ATTTCATCACTGCCA |
| | | GCCACCTCAAGAGGC |
| SEQ ID | GI 50345287 AY682848 Homo sapiens | GTCGGCTTCTCTCCC |
| NO: 4572 | receptor | TCCTTCCCCTGTACT |
| | (TNFRSF)-interacting serine- | TAGCAACAGATGGCA |
| | threonine kinase 1 | GCAGCATCTGCCCAA |
| | (RIPK1) gene, complete cds | |
| SEQ ID | GI 260593660 NG_013020 Homo sapiens | ATTAGGAGGGCTCAG |
| NO: 4573 | eosinophil peroxidase | GCCATCAACTGGAGC |
| | (EPX), RefSeqGene on chromosome 17 | TTGTGAAAGGGGTTG |
| | | CCGAAGAGAGGTGGT |
| SEQ ID | GI 1015224512 NG_023402 Homo sapiens | AGCCTCAGACCTGCA |
| NO: 4574 | Janus kinase 1 | ACATGGTTTTAAGGC |
| | (JAK1), RefSeqGene on chromosome 1 | TTTCCCTGCCCTCTC |
| | | CAGCTCCCTCTTCCT |
| SEQ ID | GI 71274112 NM_005946 Homo sapiens | CGTGCGCCTTATAGC |
| NO: 4575 | metallothionein 1A | CTCTCAACTTCTTGC |
| | (MT1A), mRNA | TTGGGATCTCCAACC |
| | | TCACCGCGGCTCGAA |
| SEQ ID | GI 289577082 NG_016598 Homo sapiens | TGCACTTCCCACATG |
| NO: 4576 | phosphorylase kinase | GCCCTAAGTGAGGTT |
| | regulatory subunit beta | GCTGGAGCAGAAGCT |
| | (PHKB), RefSeqGene on chromosome 16 | CCCAGAATCAGACCA |
| SEQ ID | GI 1015635417 AH006868 Homo sapiens | TGCAGCTTGCCGGGA |
| NO: 4577 | CD9 antigen | TTGCTGTCCTTGCCA |
| | (CD9) gene, complete cds | TTGGACTATGGCTCC |
| | | GATTCGACTCTCAGA |
| SEQ ID | GI 358679296 NG_031810 Homo sapiens | TGCGTGTCTGGTGTT |
| NO: 4578 | interferon regulatory factor 3 | GGGTGCCCTCTGACC |
| | (IRF3), RefSeqGene on chromosome 19 | CAATATGGTTGTTAT |
| | | TCCCGCTCAGGGTCT |
| SEQ ID | GI 164695782 AK315592 Homo sapiens | GCTGTAGCGAGGAGG |
| NO: 4579 | cDNA, FLI96670, Homo sapiens | ACCTGAAAGCCATCC |
| | toll interacting protein | AGGACATGTTCCCCA |
| | (TOLLIP), mRNA | ACATGGACCAGGAGG |
| SEQ ID | GI 262331544 NG_013350 Homo sapiens | TGCTTCCCTTGCCAC |
| NO: 4580 | nuclear receptor subfamily | CCGTAGGAACACTTG |
| | 3 group C member 2 | TTCTGATCCCTTCTA |
| | (NR3C2), RefSeqGene on chromosome 4 | GGAGCACAGGCTGCC |
| SEQ ID | GI 1060099004 NM_001330400 Homo sapiens | GCCAAGGGCACAGGG |
| NO: 4581 | sideroflexin 5 | AGAACCCAGATGGAG |
| | (SFXN5), transcript variant 1, mRNA | TAACTTCATGGAAGC |
| | | TGAGGACTTGGGCGA |
| SEQ ID | GI 164692348 AK314740 Homo sapiens | CCAGCCTGTTGCCAT |
| NO: 4582 | cDNA, FLI95602, Homo sapiens | GTCCCTCTCAGCAGA |
| | COMM domain containing 4 | CAAGTTCCAGGTCCT |
| | (COMMD4), mRNA | CCTGGCAGAACTGAA |
| SEQ ID | GI 164692857 AK313058 Homo sapiens | CCGTAGTGTTCGTCG |
| NO: 4583 | cDNA, FU93535, Homo sapiens | TGGGCTTGGTGGGCA |
| | opioid receptor, kappa 1 | ACTCGCTGGTCATGT |
| | (OPRK1), mRNA | TCGTGATCATCCGAT |
| SEQ ID | GI 403420573 NM_182619 Homo sapiens | GCCCAGCCTGACTCC |
| NO: 4584 | C-type lectin domain family 18 member A | TGGAGATTGTGAATA |
| | (CLEC18A), transcript variant 1, mRNA | GCTCCATCCAGCCTG |
| | | AGAAACAAGCCGGGT |
| SEQ ID | GI 1002341790 NM_000043 Homo sapiens | ACTTCTCCCATGTCA |
| NO: 4585 | Fas cell surface death receptor | ATGAGCACAGCCACA |
| | (FAS), transcript variant 1, mRNA | TTCCCGAGTTGAGGT |
| | | GACCCCACGGTCCAG |
| SEQ ID | GI 992699 U33447 Human putative | AAGCCCTGGTTCTGC |
| NO: 4586 | G-protein-coupled receptor | GTTTGCCTTGTGCTG |
| | (GPR17) gene, complete cds | AAGTTCAGAATGCCT |
| | | CTGACGCTCACGCAC |
| SEQ ID | GI 222144288 NG_009492 Homo sapiens | AGGTCTGGCCCCTTC |
| NO: 4587 | insulin like growth factor 1 receptor | ACTAGCTGCCCTCAC |
| | (IGF1R), RefSeqGene on chromosome 15 | TTCTGCAAGAAGGGA |
| | | AGAGTCAGAGCGAGA |
| SEQ ID | GI 1002095861 NG_046750 Homo sapiens | TCTGGGAAGTCCACC |
| NO: 4588 | cytochrome c oxidase subunit 8A | TCTGCAGCAGCATGT |
| | (COX8A), RefSeqGene on chromosome 11 | GTGGTGGCTAAAAGC |
| | | ATGGCTTTTGGAGGC |
| SEQ ID | GI 299758395 NG_011675 Homo sapiens | CAGTCCACCATCACT |
| NO: 4589 | glial cell derived neurotrophic factor | GATTACCGGCGCGGT |
| | (GDNF), RefSeqGene on chromosome 5 | TGCGGTGGATGCACT |
| | | TGAACCAAACCAGTG |
| SEQ ID | GI 15545992 AY029254 Homo sapiens | GTCATGCTGCCTTGT |
| NO: 4590 | Be 1-2 modifying factor | GGGGTGACTGAGGAA |
| | (BMF) mRNA, complete cds | CCCCAGCGACTCTTT |
| | | TATGGCAATGCTGGC |
| SEQ ID | GI 285026427 NG_016458 Homo sapiens | TCATCTTCCCCCACC |
| NO: 4591 | protein kinase AMP- | CCCATTTGCTGGTTC |
| | activated non-catalytic subunit gamma 3 | AGCTATGATTCAGGT |
| | (PRKAG3), RefSeqGene on chr [symbol] | AGGCTCTGCCCTGGG |
| SEQ ID | GI 238776808 NG_012244 Homo sapiens | GACCAGGTAGGGTGA |
| NO: 4592 | SMAD family member 6 | GGTTTGATTTGACAG |
| | (SMAD6), RefSeqGene on chromosome 15 | CAGCTGGCAGAGGTA |
| | | GTGCAGAGGGCTCGC |
| SEQ ID | GI 325053737 NG_028166 Homo sapiens | TTCAGGCAGTCCTGG |
| NO: 4593 | aminoacyl tRNA synthetase | GAGAAATGGATGGCT |
| | complex interacting | GGGATGTTCAAGGAA |
| | multifunctional protein 1 | CAGCAGGAGGCCGAT |
| | (AIMP1), RefSeqG [symbol] | |
| SEQ ID | GI 31563435 NM_144673 Homo sapiens | GGCCTGGCCTACCCA |
| NO: 4594 | CKLF like MARVEL | GAAACAGCAGGAGAG |
| | transmembrane domain containing 2 | AGAAGAAACAGGCCA |
| | (CMTM2), transcript variant 1, mRNA | GCTGTGAGAAGCCAA |
| SEQ ID | GI 299116025 NG_023038 Homo sapiens | ACCCCCCGCCAATTT |
| NO: 4595 | autophagy related 16 like 1 | GGTTCATCACAAAGA |
| | (ATG16L1), RefSeqGene on chromosome 2 | GTCCTGCATGGGCAT |
| | | TTTCCCACTGGGGAT |
| SEQ ID | GI 336285452 NM_006221 Homo sapiens | CGCATGAGCCGCAGC |
| NO: 4596 | peptidylprolyl cis/trans isomerase, | TCAGGCCGAGTGTAC |
| | NIMA-interacting 1 | TACTTCAACCACATC |
| | (PIN1), transcript variant 1, mRNA | ACTAACGCCAGCCAG |
| SEQ ID | GI 1147399622 NG_012448 Homo sapiens | GCTGGGACAAGGGAG |
| NO: 4597 | nodal growth differentiation factor | CATCCAGTGTGGCTT |
| | (NODAL), RefSeqGene on chromosome 10 | CTCAAGTCACAACGA |
| | | CTTAGGGCCCCTCAC |
| SEQ ID | GI 325495520 HQ692855 Homo sapiens | GGCTTCGGGGACTGG |
| NO: 4598 | nerve growth factor IB | ATTGACAGTATCCTG |
| | nuclear receptor variant 1 | GCCTTCTCAAGGTCC |
| | (NR4A1) mRNA, complete cds | CTGCACAGCTTGCTT |
| SEQ ID | GI 756140821 NM_014358 Homo sapiens | AAGAAGGAAGAGCAA |
| NO: 4599 | C-type lectin domain family 4 member E | GAACATGGCCACACC |
| | (CLEC4E), mRNA | CACCGCCCCACACGA |
| | | GAAATTTGTGCGCTG |
| SEQ ID | GI 189458805 NG_007981 Homo sapiens | TACCCAGCCTCCTTT |
| NO: 4600 | ATP binding cassette subfamily A member 1 | GTGTTCAAGATCCCC |
| | (ABCA1), RefSeqGene on chromosome 9 | AGCACAGCCTATGTG |
| | | GTGCTCACCAGCGTG |
| SEQ ID | GI 237820672 NG_012057 Homo sapiens | TGGGTGCAAAACCAC |
| NO: 4601 | carboxylesterase 1 | CACCTCTGCTGTCAT |
| | (CES1), RefSeqGene on chromosome 16 | GGTTCACTGCCTGCG |
| | | ACAGAAGACGGAAGA |
| SEQ ID | GI 38327624 NM_004077 Homo sapiens | GGCAGCAACATGGCA |
| NO: 4602 | citrate synthase | AGACGGTGGTGGGCC |
| | (CS), mRNA | AAATCACTGTGGACA |
| | | TGATGTATGGTGGCA |
| SEQ ID | GI 1036032480 AH002739 Homo sapiens | TCTGTTGCCCAGCCT |
| NO: 4603 | cholecystokinin | TTCAGTTAACGCGCC |
| | (CCK) gene, complete cds | CTCCTCCTTTGCTCC |
| | | CTACAGCCATGAACA |
| SEQ ID | GI 117606583 EF064766 Homo sapiens | GGAGCACAGTTCCTC |
| NO: 4604 | chemokine | ACTGTGGGCCCCACC |
| | (C-C motif) ligand 19 | TTTGACCATCACTTC |
| | (CCL19) gene, complete cds | TGAACCCCAGCTTCA |
| SEQ ID | GI 308522736 NG_027815 Homo sapiens | TTGTCGTGGCTTCCT |
| NO: 4605 | transketolase | ATGGACTCTAACTGA |
| | (TKT), RefSeqGene on chromosome 3 | TGTCCGGCCCCTGTT |
| | | GTGGCTCTGACCCCT |
| SEQ ID | GI 42794610 NM_012088 Homo sapiens | CCCGGGGCTCAACAC |
| NO: 4606 | 6-phosphogluconolactonase | ACAGGCTGTGGCTCT |
| | (PGLS), mRNA | GGACATCCGGATATT |
| | | AAAAGGAGCGTTGCT |
| SEQ ID | GI 8133099 AF266756 Homo sapiens | GGGCCCAGCTGCCTA |
| NO: 4607 | sphingosine kinase | TGTAAGGCCTTCTAG |
| | (SPHK1) mRNA, complete cds | TTTGTTTTGAGACCC |
| | | CCACCCCACGAACCA |
| SEQ ID | GI 194394200 NG_008275 Homo sapiens | GGGGGTTGCCAGAAG |
| NO: 4608 | prokineticin 2 | GTGTTGGGATGTGGA |
| | (PROK2), RefSeqGene on chromosome 3 | CAGGTTGAGTCACAC |
| | | TGCAGGGAGGTCTTC |
| SEQ ID | GI 323098322 NM_005624 Homo sapiens | AGGAGGTGAGCGGGA |
| NO: 4609 | C-C motif chemokine ligand 25 | GCTGCAATCTGCCTG |
| | (CCL25), transcript variant 1, mRNA | CTGCGATATTCTACC |
| | | TCCCCAAGAGACACA |
| SEQ ID | GI 223890186 NG_009797 Homo sapiens | TGCCCTTTCCCTAAC |
| NO: 4610 | junctophilin 3 | CCCATTCCCCAACAA |
| | (JPH3), RefSeqGene on chromosome 16 | GCCCTGTGGTTTTGA |
| | | CTGTGCGGTTTTGCA |
| SEQ ID | GI 62530382 NM_004281 Homo sapiens | GGTCCCAGTCACCTC |
| NO: 4611 | BCL2 associated athanogene 3 | TGCGGGGCATGCCAG |
| | (BAG3), mRNA | AAACCACTCAGCCAG |
| | | ATAAACAGTGTGGAC |
| SEQ ID | GI 22165426 NM_002991 Homo sapiens | CCAGCTGTCCAGCAG |
| NO: 4612 | C-C motif chemokine ligand 24 | GAGCACATGCCTCAA |
| | (CCL24), mRNA | GGCAGGAGTGATCTT |
| | | CACCACCAAGAAGGG |
| SEQ ID | GI 601984942 NG_034078 Homo sapiens | CACTCCTCCCCAAAT |
| NO: 4613 | myelin associated glycoprotein | TTCACCTCTCTGGTC |
| | (MAG), RefSeqGene on chromosome 19 | TTCACGATGCCCACA |
| | | ATGGCCCCCACCACA |
| SEQ ID | GI 401709925 NM_015953 Homo sapiens | GCCCAGTGTGGGTCC |
| NO: 4614 | nitric oxide synthase interacting protein | TCCAAGTAAGGACAA |
| | (NOSIP), transcript variant 1, mRNA | GGACAAAGTGCTGCC |
| | | CAGCTTCTGGATCCC |
| SEQ ID | GI 594591631 NG_034042 Homo sapiens | AGTGAAGGCCCAGCT |
| NO: 4615 | mitogen-activated protein kinase 11 | TCAAATGTGGCTGGA |
| | (MAPK11), RefSeqGene on chromosome 22 | TCCAGGCACACATCC |
| | | TGAGGTTCTGCTGGT |
| SEQ ID | GI 59853224 NM_001206 Homo sapiens | CCCAGGAAACAGACG |
| NO: 4616 | Kruppel like factor 9 | CGTGAAAACTGAGCC |
| | (KLF9), mRNA | CCAGAAGAGGCACAC |
| | | TTGACGGCACAGGAA |
| SEQ ID | GI 756761760 NM_152901 Homo sapiens | GCTCGGGCAGCTAGA |
| NO: 4617 | pyrin domain containing 1 | TATCGTGGACCTCAC |
| | (PYDC1), mRNA | CGACAAGCTGGTCGC |
| | | CTCCTACTACGAGGA |
| SEQ ID | GI 1024846751 NG_047008 Homo sapiens | AGGGGTGATGCTGCT |
| NO: 4618 | pyruvate dehydrogenase kinase 4 | GGTCCACAATGCACA |
| | (PDK4), RefSeqGene on chromosome 7 | CTTTGCCTAGCAGGC |
| | | CTGTAGAGTTGTGCT |
| SEQ ID | GI 14278710 AF374726 Homo sapiens | TTCCTGCCTCTCCCT |
| NO: 4619 | coagulation factor 11 receptor-like 2 | CACTGCACCCCAAAG |
| | (F2RL2) gene, complete cds | CCAATCAGACACCAA |
| | | GTCCCTTTAAGCCGC |
| SEQ ID | GI 95147553 NM_003956 Homo sapiens | CGCCCTTCTTCCCGG |
| NO: 4620 | cholesterol 25-hydroxylase | TCATCTTCTCCATCA |
| | (CH25H), mRNA | CCACATACGTGGGCT |
| | | TTTGCCTGCCCTTCG |
| SEQ ID | GI 40288420 AY505111 Homo sapiens | AAGACCTGGTGCCTT |
| NO: 4621 | arachidonate 15-lipoxygenase | TCCTGCTGTCAGCCC |
| | (ALOX15) gene, complete cds | TTGTTTCCTGCAGGG |
| | | ATCTTGCAGCTCTCC |
| SEQ ID | GI 45243506 NM_002935 Homo sapiens | GTTTGCCATCCAGCA |
| NO: 4622 | ribonuclease A family member 3 | CATCAGTCTGAACCC |
| | (RNASE3), mRNA | CCCTCGATGCACCAT |
| | | TGCAATGCGGGCAAT |
| SEQ ID | GI 296010927 NM_172213 Homo sapiens | CTTCGGGAAGGGAAC |
| NO: 4623 | CD8b molecule | TCAGCTGAGTGTGGT |
| | (CD8B), transcript variant 2, mRNA | TGATTTCCTTCCCAC |
| | | CACTGCCCAGCCCAC |
| SEQ ID | GI 18073363 AJ277883 Homo sapiens | ACCGCCTAGAGTGTT |
| NO: 4624 | mRNA for sialidase | TTGGCAAGATCTGCC |
| | (NEU4 gene) | GGACCAGCCCTCACT |
| | | CCTTCGCCTTCTACA |
| SEQ ID | GI 63054827 NM_001915 Homo sapiens | ACACAGCTGGTGCGG |
| NO: 4625 | cytochrome b561 | GATCCTTGTCTTTGT |
| | (CYB561), transcript variant 1, mRNA | CCTGTACTTTGTGCA |
| | | GTGGCTGGTGGGCTT |
| SEQ ID | GI 478431032 NM_003693 Homo sapiens | GCAACGCCTCCTGCC |
| NO: 4626 | scavenger receptor class F member 1 | CAGCCGGTTTCCATG |
| | (SCARF1), transcript variant 1, mRNA | GAAACAACTGCTCAG |
| | | TTCCTTGTGAATGCC |
| SEQ ID | GI 216548280 NG_009104 Homo sapiens | ATGCATCACCGTTGG |
| NO: 4627 | retinol binding protein 4 | CTCCCCAGACACTGC |
| | (RBP4), RefSeqGene on chromosome 10 | TGGGACTCAAATGGG |
| | | TGCAAGAGTGAAGGG |
| SEQ ID | GI 227430281 NM_182983 Homo sapiens | CCAGGGAATCATTAA |
| NO: 4628 | hepsin | CAAGAGGCAGTGACA |
| | (HPN), transcript variant 1, mRNA | TGGCGCAGAAGGAGG |
| | | GTGGCCGGACTGTGC |
| SEQ ID | GI 27477078 NM_013278 Homo sapiens | AGCGCTCCATCTCAC |
| NO: 4629 | interleukin 17C | CCTGGAGATACCGTG |
| | (IL17C), mRNA | TGGACACGGATGAGG |
| | | ACCGCTATCCACAGA |
| SEQ ID | GI 62865623 NM_198057 Homo sapiens | GGGGATGGCTTTGAT |
| NO: 4630 | TSC22 domain family member 3 | CCAATGGAGCCTGGG |
| | (TSC22D3), transcript variant 1, mRNA | AGGTGTGCCCAGAAA |
| | | GCTTGTCTGTAGCGG |
| SEQ ID | GI 22165426 NM_002991 Homo sapiens | GGGTCCAGAGGTACA |
| NO: 4631 | C-C motif chemokine ligand 24 | TGAAGAACCTGGACG |
| | (CCL24), mRNA | CCAAGCAGAAGAAGG |
| | | CTTCCCCTAGGGCCA |
| SEQ ID | GI 190341054 NG_008010 Homo sapiens | GGCCTTCCCACCTTC |
| NO: 4632 | aldolase, fructose-bisphosphate A | CCCACAGAGGGCTGC |
| | (ALDOA), RefSeqGene on chromosome 16 | AAGAACTTTCAAACC |
| | | TGGAGACATCAGGCC |
| SEQ ID | GI 231570519 NG_012004 Homo sapiens | GCCCAGCAAAAGCAG |
| NO: 4633 | kinase insert domain receptor | GGAGTCTGTGGCATC |
| | (KDR), RefSeqGene on chromosome 4 | TGAAGGCTCAAACCA |
| | | GACAAGCGGCTACCA |
| SEQ ID | GI 1027853651 NM_182828 Homo sapiens | AGGAGGATCACCCAC |
| NO: 4634 | growth differentiation factor 7 | ACTGGCAGGCGGGCA |
| | (GDF7), mRNA | TTTCCACTGATTCCC |
| | | AAACACACTTGCCTT |
| SEQ ID | GI 83267865 NM_001037494 Homo sapiens | GCGCCTCAGTTTCTC |
| NO: 4635 | dynein light chain LC8-type 1 | TCTGTGGCGCCGCCT |
| | (DYNLL1), transcript variant 1, mRNA | ACCTCACAGACTTGT |
| | | GAGCACTCACTGACG |
| SEQ ID | GI 169808406 NG_007450 Homo sapiens | GCCCATCCTGGGAAG |
| NO: 4636 | leptin | GAAAATGCATTGGGG |
| | (LEP), RefSeqGene on chromosome 7 | AACCCTGTGCGGATT |
| | | CTTGTGGCTTTGGCC |
| SEQ ID | GI 11691883 AB034990 Homo sapiens | GTGGCCCGAAGCAGT |
| NO: 4637 | HERPUD1 gene for stress | AGTTCTCAACTGGGG |
| | protein Herp, complete cds | GTGGTTTTGTACCCT |
| | | CTCTCCCCAGGGGAC |
| SEQ ID | GI 81230484 NM_001037161 Homo sapiens | GGGCAGAGTTGCAGG |
| NO: 4638 | acyl-CoA thioesterase 1 | GGTCTCCACAGCTGA |
| | (ACOT1), mRNA | GGCAGTTTGGCCGGA |
| | | TTATTTGGGTTCCTG |
| SEQ ID | GI 296080750 NM_002704 Homo sapiens | TCATGCCTTGCAGGT |
| NO: 4639 | pro-platelet basic protein | GCTGCTGCTTCTGTC |
| | (PPBP), mRNA | ATTGCTGCTGACTGC |
| | | TCTGGCTTCCTCCAC |
| SEQ ID | GI 315360648 NG_028024 Homo sapiens | TGACCCCCTCATCAT |
| NO: 4640 | glucokinase regulator | GCCCTTCTCTCTCTT |
| | (GCKR), RefSeqGene on chromosome 2 | CACCATCAGGCTCCC |
| | | TTTGTGGCAGGCCAG |
| SEQ ID | GI 342307092 NM_001716 Homo sapiens | CGCCAAAGTCAGCCA |
| NO: 4641 | C-X-C motif chemokine receptor 5 | AGGCCATCACAACAA |
| | (CXCR5), transcript variant 1, mRNA | CTCCCTGCCACGTTG |
| | | CACCTTCTCCCAAGA |
| SEQ ID | GI 325652058 NM_005524 Homo sapiens | GCTTCAGCGAGTGCA |
| NO: 4642 | hes family bHLH transcription factor 1 | TGAACGAGGTGACCC |
| | (HES1), mRNA | GCTTCCTGTCCACGT |
| | | GCGAGGGCGTTAATA |
| SEQ ID | GI 55775471 NM_004271 Homo sapiens | CGGTCCACCGTGGCC |
| NO: 4643 | lymphocyte antigen 86 | TGTGCCAATGCTACT |
| | (LY86), mRNA | ATCATGTGCTCCTGA |
| | | CTGTGGCCTGTAGCA |
| SEQ ID | GI 78190493 NM_013434 Homo sapiens | GGCCGCATGGCCCCT |
| NO: 4644 | potassium voltage-gated | CCAGGAACATTCCCA |
| | channel interacting protein 3 | CATAATACATTCCAT |
| | (KCNIP3), transcript variant 1, mRNA | CACAGCCAGCCCAGC |
| SEQ ID | GI 48928023 NM_003225 Homo sapiens | ACTCGGGGTCGCCTT |
| NO: 4645 | trefoil factor 1 | TGGAGCAGAGAGGAG |
| | (TFF1), mRNA | GCAATGGCCACCATG |
| | | GAGAACAAGGTGATC |
| SEQ ID | GI 1049010968 AH002726 Homo sapiens | TGGTCCCCCAGGTAG |
| NO: 4646 | c-fgr peptide | CTGAGGGCATGGCCT |
| | (FGR) gene, complete cds | ACATGGAACGCATGA |
| | | ACTACATTCACCGCG |
| SEQ ID | GI 7407497 AF243044 Homo sapiens | TGGCTGTGTACAACC |
| NO: 4647 | ECSIT | AGCTGCTCAACATCT |
| | (ECSIT) mRNA, complete cds | TCCCCAAGGAGGTCT |
| | | TCCGGCCTCGCAACA |
| SEQ ID | GI 523498696 NM_002981 Homo sapiens | AAGCTGCTCCAGGAA |
| NO: 4648 | C-C motif chemokine ligand 1 | GGCCCAAGCCAGACC |
| | (CCL1), mRNA | AGAAGACATGCAGAT |
| | | CATCACCACAGCCCT |
| SEQ ID | GI 584277161 NG_008225 Homo sapiens | CCAGCCTCTTCTGGC |
| NO: 4649 | POU class 1 homeobox 1 | AGAGGACCCCACAGC |
| | (POU1F1), RefSeqGene on chromosome 3 | TGCTGATTTCAAGCA |
| | | GGAACTCAGGCGGAA |
| SEQ ID | GI 164692348 AK314740 Homo sapiens | ACAGTGGCAGTGCTG |
| NO: 4650 | cDNA, FLI95602, Homo sapiens | AGTTTCATCCTCTCC |
| | COMM domain containing 4 | AGTGCGGCCAAGCAC |
| | (COMMD4), mRNA | AGTGTCGATGGCGAA |
| SEQ ID | GI 125661056 NM_005195 Homo sapiens | CCACCCTGGAGCTGT |
| NO: 4651 | CCAAT/enhancer binding protein delta | GCCACGACGAGCTCT |
| | (CEBPD), mRNA | TCGCCGACCTCTTCA |
| | | ACAGCAATCACAAGG |
| SEQ ID | GI 40786798 AY518533 Homo sapiens | CGGCGAAGGGCCTGG |
| NO: 4652 | interleukin 17 receptor B | CACGTAGTTATTGTT |
| | (IL17RB) gene, complete cds | ACTTGGGGTCTGCTC |
| | | ACCTGTTGGGGCACA |
| SEQ ID | GI 75677324 NM_014238 Homo sapiens | AGCTGCAAGCAAGAG |
| NO: 4653 | kinase suppressor of ras 1 | ACTGGCCCTTGAAGA |
| | (KSR1), mRNA | ACCAGGCTGCAGAGG |
| | | CATCCATCTGGCAGA |
| SEQ ID | GI 125661056 NM_005195 Homo sapiens | CGGCGCAACCAGGAG |
| NO: 4654 | CCAAT/enhancer binding protein delta | ATGCAGCAGAAGTTG |
| | (CEBPD), mRNA | GTGGAGCTGTCGGCT |
| | | GAGAACGAGAAGCTG |
| SEQ ID | GI 325652112 NM_020997 Homo sapiens | ACACCCTGGACCTTG |
| NO: 4655 | left-right determination factor 1 | GGGACTATGGAGCTC |
| | (LEFTY1), mRNA | AGGGCGACTGTGACC |
| | | CTGAAGCACCAATGA |
| SEQ ID | GI 224282141 NG_009873 Homo sapiens | ATGGGTCCGCAGACT |
| NO: 4656 | CREB binding protein | TCCTCTGCAAAGGGC |
| | (CREBBP), RefSeqGene on chromosome 16 | CAGAGAGCACGCATT |
| | | TGGCTTTCTGAGCCA |
| SEQ ID | GI 205277450 NM_001135054 Homo sapiens | GCTCCCAAAGGCGCA |
| NO: 4657 | single Ig and TIR domain containing | CCAATGACCAACATT |
| | (SIGIRR), transcript variant 1, mRNA | TGCCCCCCGGAGGAA |
| | | AGAACTGGAACCAGC |
| SEQ ID | GI 156151447 NG_006672 Homo sapiens | GGTCCTCCCACCTCA |
| NO: 4658 | actin, alpha 1, skeletal muscle | GCCTCCCCAGTTGCT |
| | (ACTA1), RefSeqGene on chromosome 1 | AATATTTTCGCATGT |
| | | GCCACCGCGTCCAGC |
| SEQ ID | GI 756761760 NM_152901 Homo sapiens | AACGAAGCGCGAGGC |
| NO: 4659 | pyrin domain containing 1 | CATCCTGAAGGTGCT |
| | (PYDC1), mRNA | GGAGAACCTGACACC |
| | | GGAGGAGCTCAAGAA |
| SEQ ID | GI 151101190 NM_006435 Homo sapiens | CCCTGCCCCCAGAGG |
| NO: 4660 | interferon induced | CCAGGAGCTCTGCCC |
| | transmembrane protein 2 | TTGACCTGTATTCCA |
| | (IFITM2), mRNA | CTTACTCCACCTTCC |
| SEQ ID | GI 18034675 AF458059 Homo sapiens | CCCACAAGGGCTACT |
| NO: 4661 | IL25 mRNA, complete cds | GCCTGGAGCGCAGGC |
| | | TGTACCGTGTTTCCT |
| | | TGGCTTGTGTGTGTG |
| SEQ ID | GI 31343499 NM_175886 Homo sapiens | GCGGCAGCTGGATCG |
| NO: 4662 | phosphoribosyl pyrophosphate | CTCGACGGAGTGCCT |
| | synthetase 1-like 1 | CTGGTAGTTGGCCAA |
| | (PRPS1L1), mRNA | GACGCCGAATATCAA |
| SEQ ID | GI 18034675 AF458059 Homo sapiens | ACCAGGTGGTTGCAT |
| NO: 4663 | IL25 mRNA, complete cds | TCTTGGCAATGGTCA |
| | | TGGGAACCCACACCT |
| | | ACAGCCACTGGCCCA |
| SEQ ID | GI 54144649 NM_002090 Homo sapiens | CTCCGGGAATTTCCC |
| NO: 4664 | C-X-C motif chemokine ligand 3 | TGGCCCGGCCGCTCC |
| | (CXCL3), mRNA | GGGCTTTCCAGTCTC |
| | | AACCATGCATAAAAA |
| SEQ ID | GI 528078360 NM_003955 Homo sapiens | AGCCTGCGCCTCAAG |
| NO: 4665 | suppressor of cytokine signaling 3 | ACCTTCAGCTCCAAG |
| | (SOCS3), mRNA | AGCGAGTACCAGCTG |
| | | GTGGTGAACGCAGTG |
| SEQ ID | GI 4455060 AF118265 Homo sapiens | CCGGGACACCATCTC |
| NO: 4666 | orphan G protein-coupled | GCGGCTGGACGGGCG |
| | receptor GPR44 | CATTATGTGCTACTA |
| | (GPR44) gene, complete cds | CAATGTGCTGCTCCT |
| SEQ ID | GI 1049010968 AH002726 Homo sapiens | TCCCCTAGAGGTGAA |
| NO: 4667 | c-fgr peptide | TGACGGGCTGTGCAA |
| | (FGR) gene, complete cds | CCTGCTCATCGCGCC |
| | | CTGCACCATCATGAA |
| SEQ ID | GI 11545840 NM_022120 Homo sapiens | GGTCCGTCGCATCGT |
| NO: 4668 | 3-oxoacid CoA-transferase 2 | CTGTTCCTACGTGGG |
| | (OXCT2), mRNA | CGAGAACACCCTGTG |
| | | CGAGAGCCAGTACCT |
| SEQ ID | GI 954170001 NM_002620 Homo sapiens | TGCTCCTGCCAGTTG |
| NO: 4669 | platelet factor 4 variant 1 | TGGTCGCCTTCGCCA |
| | (PF4V1), mRNA | GAGCTGAAGCTGAAG |
| | | AAGATGGGGACCTGC |
| SEQ ID | GI 1036032447 AH002989 Homo sapiens | CGTCCACCGGGACCT |
| NO: 4670 | pp60 c-src-1 protein | TCGTGCAGCCAACAT |
| | (SRC) gene, complete cds | CCTGGTGGGAGAGAA |
| | | CCTGGTGTGCAAAGT |
| SEQ ID | GI 49574517 NM_003695 Homo sapiens | CCTTCCCCCCAGGGA |
| NO: 4671 | lymphocyte antigen 6 family member D | AGGCCCCTCATGCCT |
| | (LY6D), mRNA | TTCCTTCCCTTTCTC |
| | | TGGGGATTCCACACC |

[symbol]

indicates data missing or illegible when filed

1. A composition comprising a PathoChip v5, wherein the PathoChip v5 comprises over 60,000 probes specific for a plurality of microbes, and a plurality of probes specific for Coronaviruses.

2. The composition of claim 1, wherein the Coronavirus probes comprise at least one nucleotide sequence selected from the group consisting of SEQ ID NOs: 1-37.

3. The composition of claim 1, wherein the Coronavirus probes consist of the nucleotide sequences set forth in SEQ ID NOs: 1-37.

4. A composition comprising a PathoChip comprising a plurality of probes specific for a plurality of respiratory pathogens, a plurality of probes specific for Coronaviruses, and a plurality of probes specific for immune markers of innate and adaptive responses to infections, inflammation, immune disorders, metabolism, and toxicological responses.

5. The composition of claim 4, wherein the plurality of probes specific for immune markers of innate and adaptive responses comprise at least one nucleotide sequence selected from the group consisting of SEQ ID NOs: 38-4,671.

6. The composition of claim 4, wherein the plurality of probes specific for immune markers of innate and adaptive responses consist of the nucleotide sequences set forth in SEQ ID NOs: 38-4,671.

7. The composition of claim 4, wherein the Coronavirus probes comprise at least one nucleotide sequence selected from the group consisting of SEQ ID NOs: 1-37.

8. The composition of claim 4, wherein the Coronavirus probes consist of the nucleotide sequences set forth in SEQ ID NOs: 1-37.

9. A composition comprising a plurality of nucleic acids, wherein the nucleic acids comprise at least one nucleotide sequence selected from the group consisting of SEQ ID NOs: 1-37.

10. A composition comprising a plurality of nucleic acids, wherein the nucleic acids consist of the nucleotide sequences set forth in SEQ ID NOs: 1-37.

11. A composition comprising a plurality of nucleic acids, wherein the nucleic acids comprise at least one nucleotide sequence selected from the group consisting of SEQ ID NOs: 38-4,671.

12. A composition comprising a plurality of nucleic acids, wherein the nucleic acids consist of the nucleotide sequences set forth in SEQ ID NOs: 38-4,671.

13. The composition of claim 9, wherein the composition comprises a microarray.

14. The composition of claim 13, wherein the microarray comprises a biochip, glass slide, bead, or paper.

15. A composition comprising an Immunome ChIP, wherein the Immunome ChIP comprises a plurality of probes specific for immune markers of innate and adaptive responses to infections, inflammation, immune disorders, metabolism, and toxicological responses.

16. The composition of claim 15, wherein the plurality of probes comprise at least one nucleotide sequence selected from the group consisting of SEQ ID NOs: 38-4,671.

17. The composition of claim 12, wherein the plurality of probes consist of the nucleotide sequences set forth in SEQ ID NOs: 38-4,671.

18. A method of detecting a Coronavirus in a sample from a subject, the method comprising:
hybridizing a detectably-labeled nucleic acid from the sample to a PathoChip v5 array, wherein the PathoChip v5 comprises over 60,000 probes specific for a plurality of microbes, and a plurality of probes specific for Coronaviruses,
wherein when the nucleic acid from the sample hybridizes to at least three Coronavirus probes on the PathoChip v5, a Coronavirus is detected in the sample.

19. A method of identifying a high-risk subject, the method comprising:
hybridizing a detectably-labeled nucleic acid from a sample from the subject to a PathoChip v5 array, wherein the PathoChip v5 comprises over 60,000 probes specific for a plurality of microbes, and a plurality of probes specific for Coronaviruses,
wherein when the nucleic acid from the sample hybridizes to at least three Coronavirus probes on the PathoChip v5 and at least three probes specific for a microbe, the subject is identified as high-risk.

20. A method of detecting a Coronavirus in a sample from a subject, the method comprising:
hybridizing a detectably-labeled nucleic acid from the sample to a PathoChip array, wherein the PathoChip comprises a plurality of probes specific for a plurality of respiratory pathogens, a plurality of probes specific for Coronaviruses, and a plurality of probes specific for immune markers of innate and adaptive responses to infections, inflammation, immune disorders, metabolism, and toxicological responses,
wherein when the nucleic acid from the sample hybridizes to at least three Coronavirus probes on the PathoChip, a Coronavirus is detected in the sample.

21. A method of identifying a high-risk subject, the method comprising:
hybridizing a detectably-labeled nucleic acid from a sample from the subject to a PathoChip array, wherein the PathoChip comprises a plurality of probes specific for a plurality of respiratory pathogens, a plurality of probes specific for Coronaviruses, and a plurality of probes specific for immune markers of innate and adaptive responses to infections, inflammation, immune disorders, metabolism, and toxicological responses,
wherein when the nucleic acid from the sample hybridizes to at least three Coronavirus probes on the PathoChip and at least three probes specific for immune markers of innate and adaptive responses, the subject is identified as high-risk.

22. The method of claim 18, wherein the Coronavirus probes comprise at least one nucleotide sequence selected from the group consisting of SEQ ID NOs: 1-37.

23. The method of claim 18, wherein the Coronavirus probes consist of the nucleotide sequences set forth in SEQ ID NOs: 1-37.

24. The method of claim 18, further comprising wherein when a Coronavirus is detected in the sample from the subject or the subject is identified as high-risk, then the subject is administered a treatment for the Coronavirus.

25. The method of claim 18, wherein the plurality of probes specific for immune markers of innate and adaptive responses comprises at least one nucleotide sequence selected from the group consisting of SEQ ID NOs: 38-4,671.

26. The method of claim 18, wherein the plurality of probes specific for immune markers of innate and adaptive responses consists of the nucleotide sequences set forth in SEQ ID NOs: 38-4,671.

27. The method of claim 18, wherein the subject is human.

28. The method of claim 18, wherein the detectably-labeled nucleic acid is labeled with a fluorophore, radioactive phosphate, biotin, or enzyme.

29. The method of claim 28, wherein the fluorophore is Cy3 or Cy5.
